(19) United States
(12) Patent Application Publication
(10) Pub. No.: US 2025/0313535 A1
(43) Pub. Date: Oct. 9, 2025

(54) ALPHA 4 BETA 7 INTEGRIN MODULATORS AND USES THEREOF

(71) Applicant: DICE MOLECULES SV, INC., Indianapolis, IN (US)

(72) Inventors: Gary Edward Lee BRANDT, Alameda (UA); Timothy James CHURCH, San Mateo, CA (US); Paul R. FATHEREE, San Francisco, CA (US); Nico Patrik GHILARDI, Redwood City, CA (US); Martin Sheringham LINSELL, San Mateo, CA (US); Liliana MOURA MASSIS, Campbell, CA (US); Maureen Kay REILLY, Emerald Hills, CA (US); Steven D.E. SULLIVAN, San Mateo, CA (US); Dandan YANG, Foster City, CA (US)

(21) Appl. No.: 18/865,632

(22) PCT Filed: May 12, 2023
(86) PCT No.: PCT/US2023/066968

§ 371 Date: Nov. 13, 2024

Related U.S. Application Data
(60) Provisional application No. 63/341,993, filed on May 13, 2022.
(60) Provisional application No. 63/497,634, filed on Apr. 21, 2023.

(51) Int. Cl. C07D 239/54; A61K 31/192; A61K 31/4035; A61K 31/4375; A61K 31/4418; A61K 31/4545; A61K 31/4704; A61K 31/4709; A61K 31/472; A61K 31/4725; A61K 31/4965; A61K 31/497; A61K 31/50; A61K 31/501; A61K 31/513; A61K 31/517; A61K 31/5375; A61K 31/5377; A61K 31/541; A61K 31/55; C07C 233/87; C07D 209/46; C07D 213/56; C07D 213/64; C07D 215/227; C07D 217/02; C07D 217/16; C07D 237/16; C07D 239/96; C07D 241/12; C07D 265/30; C07D 401/04; C07D 401/12; C07D 401/14; C07D 403/04; C07D 403/06; C07D 403/12; C07D 405/12; C07D 405/14; C07D 409/12; C07D 409/14; C07D 413/12; C07D 417/12; C07D 471/04; C07D 471/08; C07D 487/08; C07D 491/107

(52) U.S. Cl. CPC C07D 239/54; A61K 31/192; A61K 31/4035; A61K 31/4375; A61K 31/4418; A61K 31/4545; A61K 31/4704; A61K 31/4709; A61K 31/472; A61K 31/4725; A61K 31/4965; A61K 31/497; A61K 31/50; A61K 31/501; A61K 31/513; A61K 31/517; A61K 31/5375; A61K 31/5377; A61K 31/541; A61K 31/55; C07C 233/87; C07D 209/46; C07D 213/56; C07D 213/64; C07D 215/227; C07D 217/02; C07D 217/16; C07D 237/16; C07D 239/96; C07D 241/12; C07D 265/30; C07D 401/04; C07D 401/12; C07D 401/14; C07D 403/04; C07D 403/06; C07D 403/12; C07D 405/12; C07D 405/14; C07D 409/12; C07D 409/14; C07D 413/12; C07D 417/12; C07D 471/04; C07D 471/08; C07D 487/08; C07D 491/107

(57) ABSTRACT

The present disclosure provides small molecule compounds and pharmaceutical compositions for the modulation of alpha 4 beta 7 integrin, useful for the treatment of inflammatory conditions such as inflammatory bowel disease, including ulcerative colitis and Crohn's disease.

### CROSS-REFERENCE

This application claims the benefit of priority of U.S. Provisional Patent Application No. 63/341,993 filed on May 13, 2022, and U.S. Provisional Patent Application No. 63/497,634 filed on Apr. 21, 2023, each of which is incorporated herein by reference in their entireties.

### BACKGROUND

Alpha 4 beta 7 integrin (α₄ß₇), also known as Lymphocyte Peyer patch adhesion molecule (LPAM) is a powerful signaling molecule embedded in the cell membranes of immune cells. α₄ß₇ is responsible for T-cell homing into gut-associated lymphoid tissues by binding to mucosal vascular addressin cell adhesion molecule (MAdCAM) located on high endothelial venules of mucosal lymphoid organs. It has been demonstrated that α₄ß₇ is implicated in several immune system disorders, including inflammatory bowel disease (IBD) (including, e.g., Crohn's disease (CD) and ulcerative colitis (UC)) and graft-versus-host disease (GVHD).

α₄ß₇ is a clinically-validated target for IBD, with ENTYVIO (vedolizumab), an injectable anti-α₄ß₇ mAb, approved for the treatment of UC and CD. However, the accessibility of ENTYVIO is limited by the need for parenteral administration. Further, ENTYVIO causes a range of side effects (including nausea, vomiting, severe diarrhea, stomach cramps, weight loss, and pain), which can be difficult to manage due to ENTYVIO's long half-life. Therefore, there is a need for highly active and/or selective α₄ß₇ small molecule inhibitors that are orally bioavailable reduced side effects.

### SUMMARY OF THE INVENTION

In one aspect, provided herein is a compound having the structure of Formula (I), or a pharmaceutically acceptable salt or solvate, thereof:

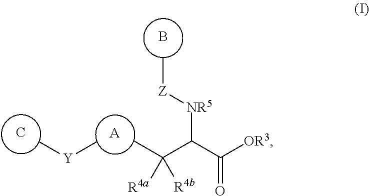

or a pharmaceutically acceptable salt thereof, wherein:
A is selected from:

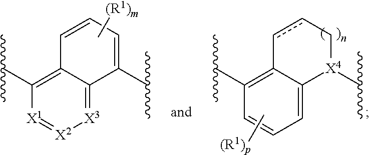

X¹, X², X³, and X⁴ are each independently selected from N and C(R²), wherein at least one of X¹, X², and X³ is C(R²);
[symbol] represents a single bond or a double bond;
m is 0, 1, 2, or 3;
n is 0, 1, or 2;
p is 0, 1, 2, or 3;
each R¹ is independently selected from:
   halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂(R¹¹), —S(O)R¹¹, —S(O)₂R¹¹, —S(O)₂N(R¹¹)₂, —NO₂, and —CN;
   C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN;
   C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
each R² is independently selected from:
   hydrogen, halogen, —OR¹², —SR¹², —N(R¹²)₂, —C(O)R¹², —C(O)OR¹², —OC(O)R¹², —OC(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂(R¹²), —S(O)R¹², —S(O)₂R¹², —S(O)₂N(R¹²)₂, —NO₂, and —CN;
   C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
   C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
   R^{4a} and R^{4b} are each independently selected from:
      hydrogen, halogen, —OR¹³, —SR¹³, —N(R¹³)₂, —C(O)R¹³, —NO₂, —CN, and C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Y is a bond, —O—, —N(R¹⁴)— or —C(R^{14a})(R^{14b})—;
Z is selected from:
   a bond, —C(O)—, —C(R¹⁵)₂—, —C(O) C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—;
   wherein q is selected from 1, 2, and 3;
B is selected from C₃₋₁₂ carbocycle and 3- to 12-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
      C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN;
C is selected from C₃₋₁₂ carbocycle and 3- to 12-membered heterocycle, each of which is optionally substituted with one or more substituents independently selected from:
   halogen, —OR¹⁸, —OR^{18A}, —SR¹⁸, —N(R¹⁸)₂, —C(O)R¹⁸, —C(O)R^{18A}, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN; and
      C₃₋₁₂ carbocycle and 3- to 12-membered heterocycle any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
   C₃₋₆ carbocycle and 3- to 6-membered heterocycle, each of which is optionally substituted with one or more substituents independently selected from halogen, —OR¹⁸, —SR¹⁸, —N(R¹⁸)₂, —C(O)R¹⁸, —NO₂, ═O, ═S, ═NR¹⁸, —N₃, —CN; and C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, ═O, and CN;
R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN;
R¹³ is independently selected at each occurrence from hydrogen and C₁₋₆ alkyl;
R^{14a} and R^{14b} are each independently selected from:
   hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, —O, and —CN; or
R^{14a} and R^{14b} are taken together to form a C₃₋₆ carbocycle or a 3- to 6-membered heterocycle, each of which is optionally substituted with one or more substituents independently selected from halogen, —OH, —NH₂, C₁₋₆ alkyl, —C₁₋₆ haloalkyl, —O—C₁₋₆ alkyl, —O—C₁₋₆ haloalkyl, —O, and —CN;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle;
R¹⁶ is selected at each occurrence from hydrogen and C₁₋₆ alkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
   C₃₋₁₀ carbocycle and 3- to 10-membered heterocycle, each of which is optionally substituted with one or more substituents independently selected from: halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and
R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl; and
R^{18A} is selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, —O, and —CN; and
   C₃₋₁₀ carbocycle and 3- to 10-membered heterocycle optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, —CN, C₃₋₆ carbocycle and 3- to 6-membered heterocycle.

In certain embodiments, the present disclosure provides a pharmaceutical composition comprising a compound or salt of Formula (I), (I-a), (II), or (III), and a pharmaceutically acceptable excipient.

In certain embodiments, the present disclosure provides a method of modulating alpha 4 beta 7 integrin in a subject in need thereof, comprising administering to the subject a compound or salt of Formula (I), (I-a), (II), or (III), or a pharmaceutical composition thereof.

In certain embodiments, the present disclosure provides a method of treating an inflammatory disease or condition to a subject in need thereof, comprising administering to the subject a compound or salt of Formula (I), (I-a), (II), or (III), or a pharmaceutical composition thereof. In some embodiments, the inflammatory disease or condition is selected from: inflammatory bowel disease, ulcerative colitis, Crohn's disease, graft-versus-host disease, type 1 diabetes, immune-mediated colitis, checkpoint inhibitor induced colitis, and primary sclerosing cholangitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

### DETAILED DESCRIPTION OF THE INVENTION

Integrin alpha 4 beta 7 is an integrin family adhesion receptor that shares subunits with alpha 4 beta 1 (VLA4) and the E-Cadherin receptor, alpha E beta 7. α₄ß₇ is critical for directing immune cells to intestinal mucosa, and is induced during T cell activation in Peyer's patches or mesenteric lymph nodes. α₄ß₇ is a clinically validated target for IBD, with selective α₄ß₇ inhibition resulting in significant anti-inflammatory effects and reduction in symptoms. However, off-target binding to α₄ß₁ can result in significant dose-limiting side effects. For example, TYSABRI (natalizumab), binds to both α₄ß₇ and α₄ß₁, and the binding of α₄ß₁ has been linked to progressive multifocal leukoencephalopathy, which resulted in the FDA restricting the use of TYSABRI in IBD.

In some aspects, the present disclosure provides an orally available α₄ß₇ integrin antagonist designed in a manner designed to mimic the anti-inflammatory actions of ENTYVIO, specifically its high selectivity for α₄ß₇ over α₄ß₁. For example, in some embodiments, the present disclosure provides compounds having over 100-fold selectivity for α₄ß₇ over α₄ß₁. In some embodiments, the present disclosure provides compounds having over 1,000-fold selectivity for α₄ß₇ over α₄ß₁.

### Ulcerative Colitis Disease

UC is a form of IBD characterized by inflammation and ulcers in the large intestine. The clinical symptoms of UC are diarrhea and bloody stool. Its clinical course is marked by exacerbations and remissions, which may occur spontaneously or in response to dietary changes, alterations in treatment regimens, other illnesses, or stress.

UC can be debilitating and can sometimes lead to life-threatening complications. Frequent diarrhea and bloody stools can lead to weight loss, dehydration and anemia. Persistent UC is associated with an increased risk of developing colon cancer. The Centers for Disease Control estimates that there are three million individuals in the United States with IBD, of which roughly half have UC. A similar number of individuals in Europe are estimated to have UC.

UC is typically treated with anti-inflammatory drugs starting with more moderate and locally delivered drugs, and progressing to systemic immunosuppressive drugs for patients with refractory disease. First line therapy for patients with mild disease consists of 5-aminosalicylates such as mesalamine and sulfasalazine. Patients with more severe disease are treated with systemic corticosteroids, with the intent of inducing remission and transitioning patients to better-tolerated drugs such as 5-aminosalicylates for maintenance. Some patients may be treated with systemic immunomodulatory drugs such as azathioprine, cyclosporine and XELJANZ (tofacitinib). Anti-inflammatory biologics such as TNFα antagonists REMICADE (infliximab), HUMIRA (adalimumab) and SIMPONI (golimumab) and the IL-12/IL-23 antagonist STELARA (ustekinumab) are effective in inducing remission in patients with moderate to severe UC.

ENTYVIO (vedolizumab), a monoclonal antibody that selectively targets α₄ß₇, was first approved by the FDA to treat UC and CD in 2014. In clinical trials, approximately 30% of patients receiving ENTYVIO achieved remission at the end of one year of treatment. ENTYVIO is administered as a 30-minute intravenous infusion at zero, two and six weeks, then every eight weeks thereafter. Long term therapy is generally well-tolerated in patients, but frequent dose adjustments have been reported to be required to maintain efficacy.

### Crohn's Disease Background

CD is a chronic inflammatory disease that most commonly affects the end of the small intestine and the beginning of the large intestine, although it may involve any part of the gastrointestinal tract. Both CD and UC are types of IBD and many of the symptoms and demographics overlap. In addition to the potential of CD developing in other segments of the intestine, CD differs from UC in that there can be normal healthy tissue in between patches of diseased tissue in CD, unlike UC where the inflammation is continuous. CD can also occur in all layers of the intestinal wall unlike UC which is limited to the inner most layer. It is estimated that there are 1.5 million individuals in the United States and 1.1 million individuals in Europe with CD.

The treatment paradigm for CD is very similar to that of UC with currently approved therapies focused on anti-inflammatory agents. Nearly 60% of CD patients will require surgery within twenty years of diagnosis to treat complications such as fistulas, or abnormal connections between body parts, life-threatening bleeding and intestinal obstructions.

While there are numerous approved therapeutics for UC and CD, there remains a significant unmet medical need for patients and clinicians to effectively and conveniently manage these chronic diseases, which could be facilitated by effective oral therapies.

In some aspects, the compounds of the present disclosure are used for the treatment and/or prevention of IBD. In some aspects of the present disclosure, the compounds provided herein are used for the treatment and/or prevention of UC. In some aspects of the present disclosure, the compounds provided herein are used for the treatment and/or prevention of CD.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene-refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y}, alkenyl” and “C_{x-y} alkynyl” refer to unsaturated aliphatic groups analogous in length and possible substitution to the alkyls described above, but that contain at least one double or triple bond, respectively. The term —C_{x-y} alkenylene-refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term-Cy-alkynylene-refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3 to 12 membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2oxopiperazinyl, 2oxopiperidinyl, 2oxopyrrolidinyl, oxazolidinyl, piperidinyl, piperazinyl, 4piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1oxothiomorpholinyl, and 1,1dioxothiomorpholinyl. Heterocycloalkyl may be optionally substituted by one or more substituents such as those substituents described herein.

The term “heteroaryl” refers to a radical derived from a 3 to 12 membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula-O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro, 2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In one aspect, provided herein is a compound having the structure of Formula (I), or a pharmaceutically acceptable salt or solvate, thereof:

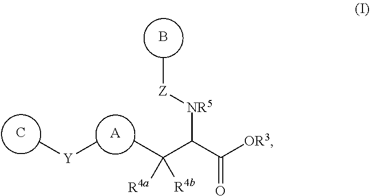

or a pharmaceutically acceptable salt thereof, wherein:
A is selected from:

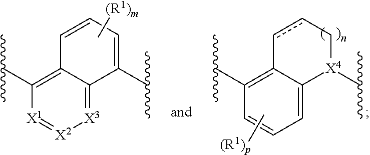

X¹, X², X³, and X⁴ are each independently selected from N and C(R²), wherein at least one of X¹, X², and X³ is C(R²);
[symbol] represents a single bond or a double bond;
m is 0, 1, 2, or 3;
n is 0, 1, or 2;
p is 0, 1, 2, or 3;
each R¹ is independently selected from:
   halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂(R¹¹), —S(O)R¹¹, —S(O)₂R¹¹, —S(O)₂N(R¹¹)₂, —NO₂, and —CN;
   C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN;
   C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
each R² is independently selected from:
   hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂(R¹²), —S(O)R¹², —S(O)₂R¹², —S(O)₂N(R¹²)₂, —NO₂, and —CN;
   C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
   C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
   R^{4a} and R^{4b} are each independently selected from:
      hydrogen, halogen, —OR¹³, —SR¹³, —N(R¹³)₂, —C(O)R¹³, —NO₂, —CN, and C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Y is a bond, —O—, —N(R¹⁴)— or —C(R^{14a})(R^{14b})—;
Z is selected from:
   a bond, —C(O)—, —C(R¹⁵)₂—, —C(O) C(R¹⁵)₂—, —C(O) [C(R¹⁵)₂]_{q}S—, —C(═N—CN)—,
   —C(O)O—, —C(O)N(R¹⁶)—, —C(S)—, —C(S)N(R¹⁶)—, —S(O)—, and —S(O)₂—;
   wherein q is selected from 1, 2, and 3;
B is selected from C₃₋₁₂ carbocycle and 3- to 12-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
      C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN;
C is selected from C₃₋₁₂ carbocycle and 3- to 12-membered heterocycle, each of which is optionally substituted with one or more substituents independently selected from:
   halogen, —OR¹⁸, —OR^{18A}, —SR¹⁸, —N(R¹⁸)₂, —C(O)R¹⁸, —C(O)R^{18A}, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN; and
      C₃₋₁₂ carbocycle and 3- to 12-membered heterocycle any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}—N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₆ alkyl, C₁₋₆ haloalkyl, C₃₋₆ carbocycle, —C₃₋₆ carbocycle-(C)₁₋₆ haloalkyl), and 3- to 6-membered heterocycle; and
   C₃₋₆ carbocycle and 3- to 6-membered heterocycle, each of which is optionally substituted with one or more substituents independently selected from halogen, —OR¹⁸, —SR¹⁸, —N(R¹⁸)₂, —C(O)R¹⁸, —NO₂, ═O, ═S, ═NR¹⁸, —N₃, —CN; and C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, ═O, and CN;
R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, —O, and —CN;
R¹³ is independently selected at each occurrence from hydrogen and C₁₋₆ alkyl;
R^{14a} and R^{14b} are each independently selected from:
   hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, —O, and —CN; or
R^{14a} and R^{14b} are taken together to form a C₃₋₆ carbocycle or a 3- to 6-membered heterocycle, each of which is optionally substituted with one or more substituents independently selected from halogen, —OH, —NH₂, C₁₋₆ alkyl, —C₁₋₆ haloalkyl, —O—C₁₋₆ alkyl, —O—C₁₋₆ haloalkyl, —O, and —CN;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle;
R¹⁶ is selected at each occurrence from hydrogen and C₁₋₆ alkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
   C₃₋₁₀ carbocycle and 3- to 10-membered heterocycle, each of which is optionally substituted with one or more substituents independently selected from: halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl; and
R^{18A} is selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, —CN, C₃₋₆ carbocycle and 3- to 6-membered heterocycle.

In some embodiments, B is optionally substituted 3- to 12-membered heterocycle. In some embodiments, B is optionally substituted 3- to 12-membered saturated heterocycle. In some embodiments, B is optionally substituted 3- to 12-membered unsaturated heterocycle. In some embodiments, B is optionally substituted 6- to 12-membered aromatic heterocycle. In some embodiments, B is selected from 3-membered heterocycle, 4-membered heterocycle, 5-membered heterocycle, 6-membered heterocycle, 7-membered heterocycle, 8-membered heterocycle, 9-membered heterocycle, 10-membered heterocycle, 11-membered heterocycle, and 12-membered heterocycle, any one of which is optionally substituted. In some embodiments, B is selected from 3- to 4-membered heterocycle, 3- to 5-membered heterocycle, 3- to 6-membered heterocycle, 3- to 7-membered heterocycle, 3- to 8-membered heterocycle, 3- to 9-membered heterocycle, 3- to 10-membered heterocycle, 3- to 11-membered heterocycle, and 3- to 12-membered heterocycle, any one of which is optionally substituted. In some embodiments, B is selected from 3- to 8-membered monocyclic heterocycle and 6- to 12-membered bicyclic heterocycle, any one of which is optionally substituted. In some embodiments, B is optionally substituted 3- to 8-membered monocyclic heterocycle. In some embodiments, B is optionally substituted 6- to 12-membered bicyclic heterocycle. In some embodiments, the optionally substituted 6- to 12-membered bicyclic heterocycle of B is selected from 6- to 12-membered bridged heterocycle, 6- to 12-membered spirocyclic heterocycle, and 6- to 12-membered fused heterocycle, any one of which is optionally substituted. In some embodiments, the optional substituents of B are as defined herein. In some embodiments, the optional substituents of B are as defined in Formula (I).

In some embodiments, B is optionally substituted C₃₋₁₂ carbocycle. In some embodiments, B is optionally substituted saturated C₃₋₁₂ carbocycle. In some embodiments, B is optionally substituted unsaturated C₃₋₁₂ carbocycle. In some embodiments, B is optionally substituted aromatic C₈₋₁₂ carbocycle. In some embodiments, B is selected from C₃ carbocycle, C₄ carbocycle, C₅ carbocycle, C₆ carbocycle, C₇ carbocycle, C₈ carbocycle, C₉ carbocycle, C₁₀ carbocycle, C₁₀ carbocycle, C₁₁ carbocycle, and C₁₂ carbocycle, any one of which is optionally substituted. In some embodiments, B is selected from C₃₋₄ carbocycle, C₃₋₅ carbocycle, C₃₋₆ carbocycle, C₃₋₇ carbocycle, C₃₋₈ carbocycle, C₃₋₉ carbocycle, C₃₋₁₀ carbocycle, C₃₋₁₁ carbocycle, and C₃₋₁₂carbocycle, any one of which is optionally substituted. In some embodiments, B is selected from monocyclic C₃₋₈ carbocycle and bicyclic C₆₋₁₂ carbocycle, any one of which is optionally substituted. In some embodiments, the optional substituents of B are as defined herein. In some embodiments, the optional substituents of B are as defined in Formula (I).

In some embodiments, for the compound or salt of Formula (I), B is 3- to 12-membered heterocycle optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN.

In some embodiments, for the compound or salt of Formula (I), B is 3- to 12-membered heterocycle optionally substituted with one or more substituents independently selected from:
halogen, —OR¹⁷, —SR¹⁷, —N(R¹⁷)₂, —C(O)R¹⁷, —C(O)OR¹⁷, —NO₂, —CN;
C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, —CN, C₃₋₁₀ carbocycle and 3- to 10-membered heterocycle, wherein the C₃₋₁₀ carbocycle and 3- to 10-membered heterocycle are each optionally substituted with one or more substituents independently selected from: halogen, —OR¹⁷, —SR¹⁷, —N(R¹⁷)₂, —C(O)R¹⁷, —C(O)OR¹⁷, —NO₂, —CN; and
C₃₋₁₀ carbocycle and 3- to 10-membered heterocycle, each of which is optionally substituted with one or more substituents independently selected from: halogen, —OR¹⁷, —SR¹⁷, —N(R¹⁷)₂, —C(O)R¹⁷, —C(O)OR¹⁷, —NO₂, —CN; and C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —NO₂, and —CN.

In some embodiments, B is 6- to 12-membered bicyclic heterocycle selected from 6- to 12-membered fused heterocycle, 6- to 12-membered spirocyclic heterocycle, and 6- to 12-membered bridged heterocycle, each of which is optionally substituted with one or more substituents independently selected from halogen, —OR¹⁷, —SR¹⁷, —N(R¹⁷)₂, —C(O)R¹⁷, —C(O)OR¹⁷, —NO₂, —CN; and C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, and —CN. In some embodiments, B is 6- to 12-membered bridged heterocycle optionally substituted with one or more substituents independently selected from halogen, —OR¹⁷, —SR¹⁷, —N(R¹⁷)₂, —C(O)R¹⁷, —C(O)OR¹⁷, —NO₂, —CN; and C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, and —CN. In some embodiments, B is 6- to 12-membered fused heterocycle optionally substituted with one or more substituents independently selected from halogen, —OR¹⁷, —SR¹⁷, —N(R¹⁷)₂, —C(O)R¹⁷, —C(O)OR¹⁷, —NO₂, —CN; and C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, and —CN. In some embodiments, B is 6- to 12-membered spirocyclic heterocycle optionally substituted with one or more substituents independently selected from halogen, —OR¹⁷, —SR¹⁷, —N(R¹⁷)₂, —C(O)R¹⁷, —C(O)OR¹⁷, —NO₂, —CN; and C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, and —CN. In some embodiments, B is 9- to 12-membered spirocyclic heterocycle optionally substituted with one or more substituents independently selected from halogen, —OR¹⁷, —N(R¹⁷)₂, —C(O)R¹⁷, —NO₂, —CN and C₁₋₆ alkyl. In some embodiments, B is

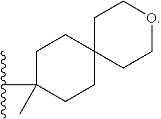 [SMILES: *C1(C)CCC2(CCOCC2)CC1]

In some embodiments, C is optionally substituted C₃₋₁₂ carbocycle. In some embodiments, C is optionally substituted saturated C₃₋₁₂ carbocycle. In some embodiments, C is optionally substituted unsaturated C₃₋₁₂ carbocycle. In some embodiments, C is optionally substituted aromatic C₈₋₁₂ carbocycle. In some embodiments, C is selected from C₃ carbocycle, C₄ carbocycle, C₅ carbocycle, C₆ carbocycle, C₇ carbocycle, C₈ carbocycle, C₉ carbocycle, C₁₀ carbocycle, C₁₀ carbocycle, C₁₁ carbocycle, and C₁₂ carbocycle, any one of which is optionally substituted. In some embodiments, C is selected from C₃₋₄ carbocycle, C₃₋₅ carbocycle, C₃₋₆ carbocycle, C₃₋₇ carbocycle, C₃₋₈ carbocycle, C₃₋₉ carbocycle, C₃₋₁₀ carbocycle, C₃₋₁₁ carbocycle, and C₃₋₁₂carbocycle, any one of which is optionally substituted. In some embodiments, C is selected from C₃₋₈ monocyclic carbocycle and C₆₋₁₂ bicyclic carbocycle, any one of which is optionally substituted. In some embodiments, C is optionally substituted C₃₋₈ monocyclic carbocycle. In some embodiments, C is optionally substituted C₆₋₁₂ bicyclic carbocycle. In some embodiments, the C₆₋₁₂ bicyclic carbocycle of C is selected from C₆₋₁₂ spirocyclic carbocycle, C₆₋₁₂ fused carbocycle, and C₆₋₁₂ bridged carbocycle, any one of which is optionally substituted. In some embodiments, the optional substituents of C are as defined herein. In some embodiments, the optional substituents of Care as defined in Formula (I).

In some embodiments, for the compound or salt of Formula (I), C is selected from C₃₋₁₀ carbocycle and 3- to 10-membered heterocycle, optionally substituted with —OR^{18A} or —C(O)R^{18A}; and R^{18A} is selected from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, and —CN; and
C₃₋₆ carbocycle and 3- to 6-membered heterocycle optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, —CN, C₃₋₆ carbocycle and 3- to 6-membered heterocycle.

In some embodiments, for the compound or salt of Formula (I), C is selected from C₃₋₁₀ carbocycle and 3- to 10-membered heterocycle, optionally substituted with —OR^{18A} or —C(O)R^{18A}; R^{18A} is selected from and R^{18A} is selected from C₁₋₆ alkyl optionally substituted with one more substituents independently selected from halogen, C₃₋₁₀ carbocycle and 3- to 10-membered heterocycle. In some embodiments, C is selected from C₃₋₁₀ carbocycle and 3- to 10-membered heterocycle, optionally substituted with —OR^{18A} or —C(O)R^{18A}; R^{18A} is selected from C₁₋₆ alkyl optionally substituted with one more substituents independently selected from halogen, C₃₋₁₀ carbocycle and 3- to 10-membered heterocycle. In some embodiments, C is C₃₋₁₀ carbocycle and 3- to 10-membered heterocycle, optionally substituted with —OR^{18A} or —C(O)R^{18A}; R^{18A} is selected from C₃₋₆ carbocycle and 3- to 6-membered heterocycle optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, —CN, C₃₋₆ carbocycle and 3- to 6-membered heterocycle. In some embodiments, C is selected from

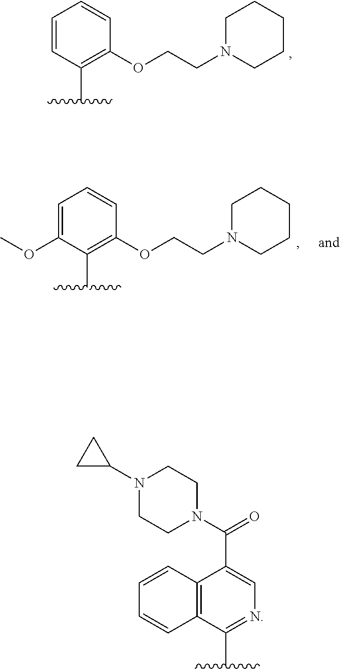 [SMILES: *c1ccccc1OCCN1CCCCC1 ; *c1c(OC)cccc1OCCN1CCCCC1 ; *c1ncc(C(=O)N2CCN(C3CC3)CC2)c2ccccc12]

In some embodiments, for the compound or salt of Formula (I), C is C₃₋₁₂ carbocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN; and
   C₃₋₁₂ carbocycle and 3- to 12-membered heterocycle, any of which is optionally substituted with one or more substituents independently selected from:
      halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
   halogen, —OR¹⁸, —SR¹⁸, —N(R¹⁸)₂, —C(O)R¹⁸, —NO₂, ═O, ═S, ═NR¹⁸, —N₃, —CN; and
   C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, and —CN.

In some embodiments, for the compound or salt of Formula (I), C is C₃₋₁₀ carbocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN;
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
   halogen, —OR¹⁸, —SR¹⁸, —N(R¹⁸)₂, —C(O)R¹⁸, —NO₂, and —CN; and
   C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₃₋₆ carbocycle and 3- to 6-membered heterocycle, wherein C₃₋₆ carbocycle and 3- to 6-membered heterocycle, are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN.

In some embodiments, for the compound or salt of Formula (I), C is C₃₋₆ carbocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN;
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
   halogen, —OR¹⁸, —SR¹⁸, —N(R¹⁸)₂, —C(O)R¹⁸, —NO₂, and —CN; and
   C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₃₋₆ carbocycle and 3- to 6-membered heterocycle, wherein C₃₋₆ carbocycle and 3- to 6-membered heterocycle, are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN.

In some embodiments, for the compound or salt of Formula (I), C is phenyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from: halogen, —OR¹⁸, —N(R¹⁸)₂, —NO₂, and —CN; and 3- to 12-membered saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from: halogen, —OR¹⁸, —N(R¹⁸)₂, —NO₂, and —CN; and 3- to 7-membered heterocycloalkyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, —CN; and
   azetidinyl, piperidinyl, piperazinyl, and azapanyl, each of which is optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is phenyl optionally substituted with one or more substituents independently selected from: halogen, —OR¹⁸, —N(R¹⁸)₂, —NO₂, and —CN; and C₁₋₆ alkyl optionally substituted with piperidinyl, piperazinyl, and azapanyl, each of which is optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and each of R¹⁸ and R^{18A} are independently selected at each occurrence from hydrogen, halogen, C₁₋₆ alkyl, and C₁₋₆ haloalkyl. In some embodiments, C is selected from:

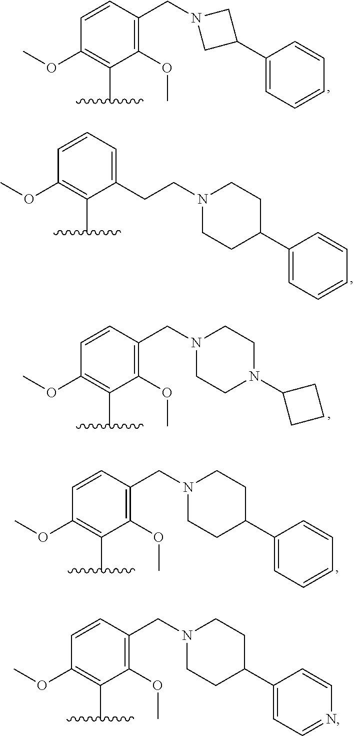

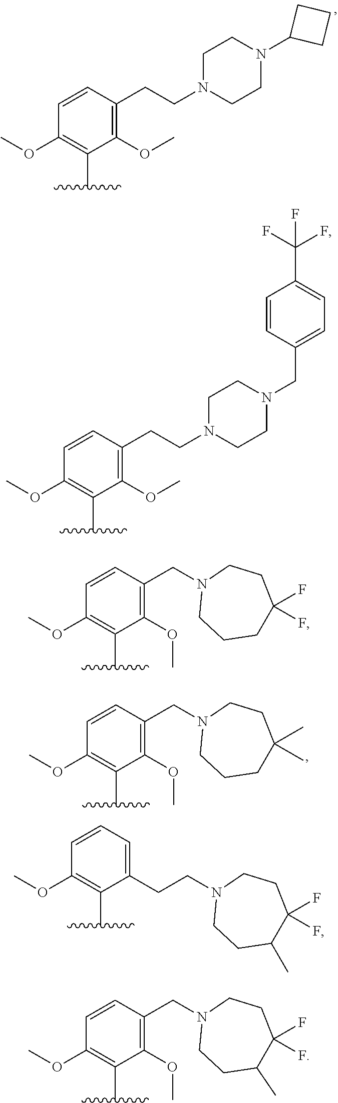

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, and —CN; and
   8- to 12-membered spirocyclic saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle.

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, and —CN; and
   6-azaspiro[2.5]octanyl, 2-oxa-8-azaspiro[4.5]decanyl, 3-azaspiro[5.5]undecanyl, and 2,9-diazaspiro[5.5]undecanyl, each of which is optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is phenyl optionally substituted with one or more substituents independently selected from: halogen, —OR¹⁸, —N(R¹⁸)₂, —NO₂, and —CN; and C₁₋₆ alkyl optionally substituted with one or more 6-azaspiro[2.5]octanyl, 2-oxa-8-azaspiro[4.5]decanyl, 3-azaspiro[5.5]undecanyl, and 2,9-diazaspiro[5.5]undecanyl, each of which is optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl. In some embodiments, C is selected from:

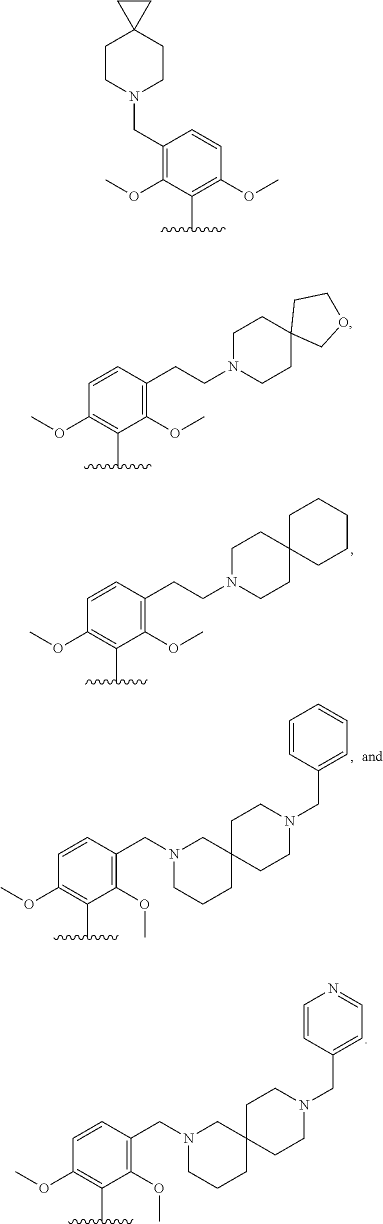

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, and —CN; and
   8- to 10-membered bridged saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle.

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, and —CN; and
   8-oxa-3-azabicyclo[3.2.1]octanyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is phenyl optionally substituted with one or more substituents independently selected from:
halogen, —OR¹⁸, —N(R¹⁸)₂, —NO₂, and —CN; and C₁₋₆ alkyl optionally substituted with 8-oxa-3-azabicyclo[3.2.1]octanyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is phenyl optionally substituted with one or more substituents independently selected from: —OR¹⁸, —CN, and C₁₋₆ alkyl optionally substituted with 8-oxa-3-azabicyclo[3.2.1]octanyl optionally substituted with substituents independently selected from: halogen, —OR^{18A}, —NO₂, —CN, and C₁₋₆ alkyl. In some embodiments, C is selected from

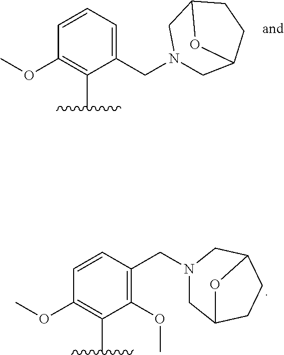 [SMILES: *c1c(CN2CC3CCC(C2)O3)cccc1OC ; *c1c(OC)ccc(CN2CC3CCC(C2)O3)c1OC]

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, and —CN; and
   6- to 10-membered fused saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle.

In some embodiments, for the compound or salt of Formula (I), C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
   halogen, —OR¹⁸, —N(R¹⁸)₂, —NO₂, and —CN; and
   octahydro-1H-isoindolyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is phenyl optionally substituted with one or more substituents independently selected from halogen, —OR¹⁸, —N(R¹⁸)₂, —NO₂, and —CN; and C₁₋₆ alkyl optionally substituted with one or more substituents independently selected from: octahydro-1H-isoindolyl optionally substituted with one or more substituents independently selected from: halogen, —OR^{18A}, —NO₂, —CN, and C₁₋₆ alkyl. In some embodiments, C is

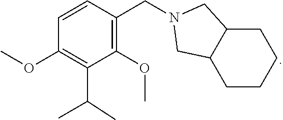 [SMILES: COc1ccc(CN2CC3CCCCC3C2)c(OC)c1C(C)C]

In some embodiments, for the compound or salt of Formula (I), C is C₆₋₁₂ carbocycle optionally substituted with one or more substituents independently selected from C₃₋₆ carbocycle and 3- to 6-membered heterocycle, each of which is optionally substituted with one or more substituents independently selected from:
halogen, —OR¹⁸, —SR¹⁸, —N(R¹⁸)₂, —C(O)R¹⁸, —NO₂, ═O, ═S, ═NR¹⁸, —N₃, —CN; and
C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, and —CN.

In some embodiments, for the compound or salt of Formula (I), C is C₈₋₁₂ fused carbocycle optionally substituted with one or more substituents independently selected from 5- to 6-membered heteroaryl optionally substituted with one or more substituents independently selected from:
halogen, —OR¹⁸, —SR¹⁸, —N(R¹⁸)₂, —NO₂, —CN; and
C₁₋₆ alkyl optionally substituted with one or more substituents independently selected from halogen, —OR^{18A}, —N(R^{18A})₂, —NO₂, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —N(R^{18A})₂, —NO₂, —CN; and each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is C₈₋₁₂ fused carbocycle optionally substituted with one or more substituents independently selected from 5- to 6-membered heteroaryl optionally substituted with one or more substituents independently selected from C₁₋₆ alkyl optionally substituted with one or more substituents independently selected from halogen, C₃₋₆ carbocycle and 3- to 6-membered heterocycle, wherein the C₃₋₆ carbocycle and 3- to 6-membered heterocycle are each optionally substituted with one or more substituents independently selected from halogen. In some embodiments, for the compound or salt of Formula (I), C is

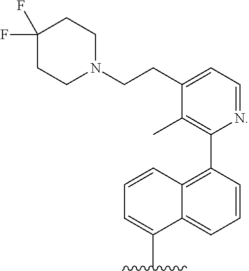 [SMILES: *c1cccc2c(-c3nccc(CCN4CCC(F)(F)CC4)c3C)cccc12]

In some embodiments, C is optionally substituted 3- to 12-membered heterocycle. In some embodiments, C is optionally substituted 3- to 12-membered saturated heterocycle. In some embodiments, C is optionally substituted 3- to 12-membered unsaturated heterocycle. In some embodiments, C is optionally substituted 6- to 12-membered aromatic heterocycle. In some embodiments, C is selected from 3-membered heterocycle, 4-membered heterocycle, 5-membered heterocycle, 6-membered heterocycle, 7-membered heterocycle, 8-membered heterocycle, 9-membered heterocycle, 10-membered heterocycle, 11-membered heterocycle, and 12-membered heterocycle, any one of which is optionally substituted. In some embodiments, C is selected from 3- to 4-membered heterocycle, 3- to 5-membered heterocycle, 3- to 6-membered heterocycle, 3- to 7-membered heterocycle, 3- to 8-membered heterocycle, 3- to 9-membered heterocycle, 3- to 10-membered heterocycle, 3- to 11-membered heterocycle, and 3- to 12-membered heterocycle, any one of which is optionally substituted. In some embodiments, C is selected from 3- to 8-membered monocyclic heterocycle and 6- to 12-membered bicyclic heterocycle, any one of which is optionally substituted. In some embodiments, C is optionally substituted 3- to 8-membered monocyclic heterocycle. In some embodiments, C is optionally substituted 6- to 12-membered bicyclic heterocycle. In some embodiments, the optionally substituted 6- to 12-membered bicyclic heterocycle of C is selected from 6- to 12-membered bridged heterocycle, 6- to 12-membered spirocyclic heterocycle, and 6- to 12-membered fused heterocycle, any one of which is optionally substituted. In some embodiments, the optional substituents of C are as defined herein. In some embodiments, the optional substituents of C are as defined in Formula (I).

In some embodiments, for the compound or salt of Formula (I), C is 3- to 12-membered heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN; and
   C₃₋₁₂ carbocycle and 3- to 12-membered heterocycle, any of which is optionally substituted with one or more substituents independently selected from:
      halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
   halogen, —OR¹⁸, —SR¹⁸, —N(R¹⁸)₂, —C(O)R¹⁸, —NO₂, ═O, ═S, ═NR¹⁸, —N₃, —CN; and
   C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂(R^{18A}), —S(O)R^{18A}, —S(O)₂R^{18A}, —S(O)₂N(R^{18A})₂, —NO₂, ═O, ═S, ═NR^{18A}, —N₃, and —CN.

In some embodiments, for the compound or salt of Formula (I), C is pyridinyl, optionally substituted with one or more substituents independently selected from:
halogen, —OR¹⁸, —N(R¹⁸)₂, —C(O)R¹⁸, —NO₂, and —CN; and
C₁₋₆ alkyl, optionally substituted with one or more substituents independently selected from:
   halogen, —OR¹⁸, —N(R¹⁸)₂, —C(O)R¹⁸, —NO₂, and —CN; and
   C₃₋₁₂ carbocycle and 3- to 12-membered heterocycle, any of which is optionally substituted with one or more substituents independently selected from:
      halogen, —OR^{18A}, —N(R^{18A})₂, —C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
each of R¹⁸ and R^{18A} are independently selected at each occurrence from hydrogen, halogen, C₁₋₆ alkyl, and C₁₋₆ haloalkyl.

In some embodiments, for the compound or salt of Formula (I), C is pyridinyl, optionally substituted with one or more substituents independently selected from:
halogen, —OR¹⁸, —NO₂, and —CN; and C₁₋₆ alkyl optionally substituted with one or more substituents independently selected from C₃₋₁₂ carbocycle and 3- to 12-membered heterocycle, any of which is optionally substituted with one or more substituents independently selected from:
   halogen, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂—NO₂, —CN, C₁₋₆ alkyl, C₁₋₆ haloalkyl; and each of R¹⁸ and R^{18A} are independently selected at each occurrence from hydrogen, halogen, C₁₋₆ alkyl, and C₁₋₆ haloalkyl. In some embodiments, C is selected from:

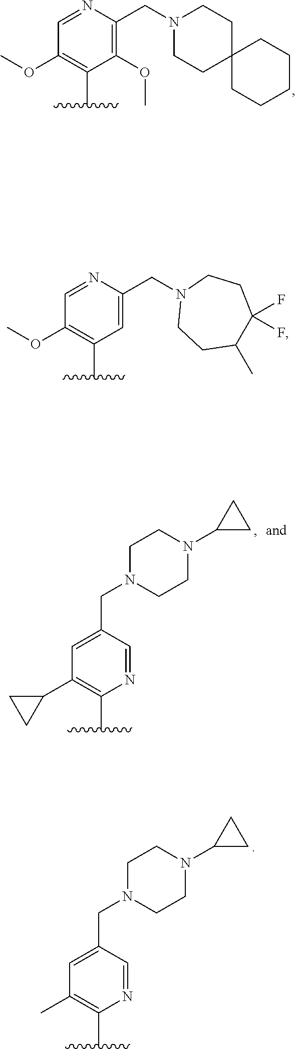 [SMILES: *c1c(OC)cnc(CN2CCC3(CCCCC3)CC2)c1OC ; *c1cc(CN2CCC(C)C(F)(F)CC2)ncc1OC ; *c1ncc(CN2CCN(C3CC3)CC2)cc1C1CC1 ; *c1ncc(CN2CCN(C3CC3)CC2)cc1C]

In some embodiments, for the compound or salt of Formula (I), C is selected from a 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN;
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
   halogen, —OR¹⁸, —SR¹⁸, —N(R¹⁸)₂, —C(O)R¹⁸, —NO₂, and —CN; and
   C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₃₋₆ carbocycle and 3- to 6-membered heterocycle, wherein C₃₋₆ carbocycle and 3- to 6-membered heterocycle, are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN.

In some embodiments, for the compound or salt of Formula (I), C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
   C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   3- to 8-membered monocyclic heterocycloalkyl optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is 8- to 12-membered bicyclic heterocycle optionally substituted with one or more substituents independently selected from:
halogen, —OR¹⁸, —N(R¹⁸)₂, —C(O)R¹⁸, ═O, —NO₂, and —CN; and
C₁₋₆ alkyl optionally substituted with one or more substituents independently selected from:
   halogen, —OR¹⁸, —N(R¹⁸)₂, —C(O)R¹⁸, —NO₂, and —CN; and
   3- to 8-membered monocyclic heterocycloalkyl optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle.

In some embodiments, for the compound or salt of Formula (I), C is 8- to 12-membered bicyclic heterocycle optionally substituted with one or more substituents independently selected from: halogen, —OR¹⁸, —N(R¹⁸)₂, —C(O)R¹⁸, ═O, —NO₂, and —CN; and C₁₋₆ alkyl optionally substituted with one or more substituents independently selected from: 3- to 8-membered monocyclic heterocycloalkyl optionally substituted with one or more substituents independently selected from: halogen, C₁₋₆ alkyl, C₃₋₆ carbocycle, and 3- to 6-membered heterocycle. In some embodiments, C is selected from

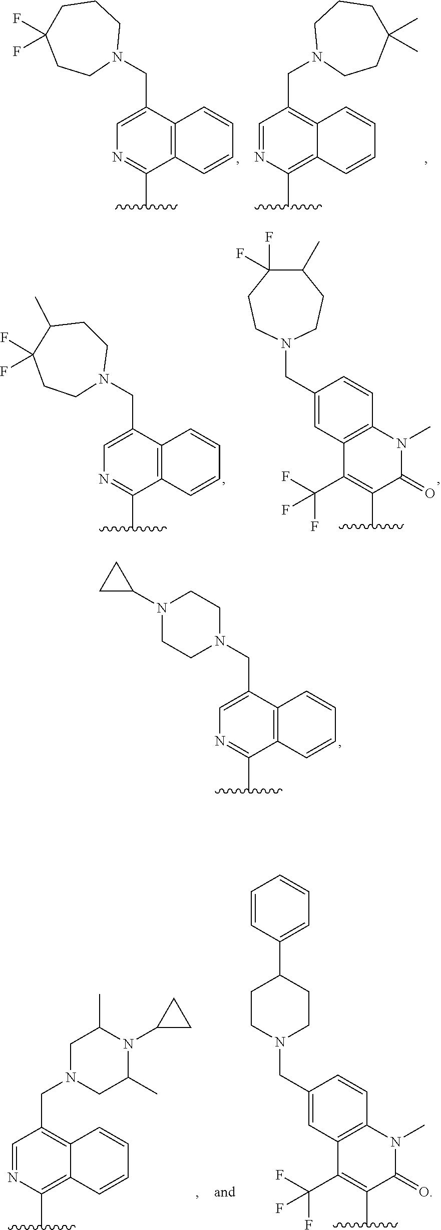

In some embodiments, for the compound or salt of Formula (I), C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   3- to 12-membered bicyclic heterocycle optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   6- to 12-membered spirocyclic heterocycle optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is 8- to 12-membered bicyclic heterocycle optionally substituted with one or more substituents independently selected from:
halogen, —OR¹⁸, —N(R¹⁸)₂, —C(O)R¹⁸, ═O, —NO₂, and —CN; and
C₁₋₆ alkyl optionally substituted with one or more substituents independently selected from:
   halogen, —OR¹⁸, —N(R¹⁸)₂, —C(O)R¹⁸, —NO₂, and —CN; and
   6- to 12-membered spirocyclic heterocycle optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle.

In some embodiments, for the compound or salt of Formula (I), C is 8- to 12-membered bicyclic heterocycle optionally substituted with one or more substituents independently selected from C₁₋₆ alkyl optionally substituted with one or more substituents independently selected from: 6- to 12-membered spirocyclic heterocycle optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle. In some embodiments, C is selected from

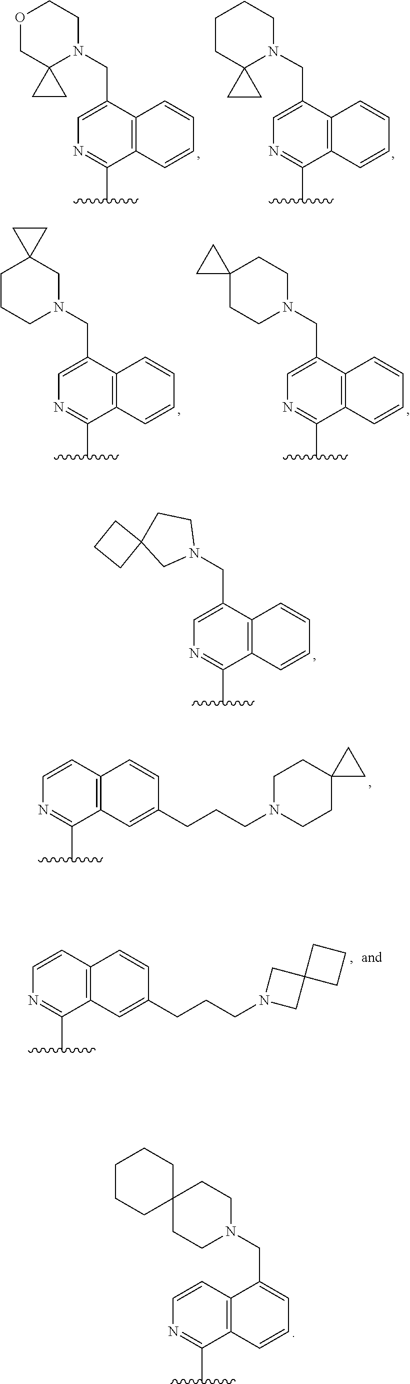

In some embodiments, for the compound or salt of Formula (I), C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
   6- to 12-membered bridged heterocycle optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

In some embodiments, for the compound or salt of Formula (I), C is 8- to 12-membered bicyclic heterocycle optionally substituted with one or more substituents independently selected from:
halogen, —OR¹⁸, —N(R¹⁸)₂, —C(O)R¹⁸, ═O, —NO₂, and —CN; and
C₁₋₆ alkyl optionally substituted with one or more substituents independently selected from:
   halogen, —OR¹⁸, —N(R¹⁸)₂, —C(O)R¹⁸, —NO₂, and —CN; and
   6- to 12-membered bridged heterocycle optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle.

In some embodiments, for the compound or salt of Formula (I), C is 8- to 12-membered bicyclic heterocycle optionally substituted with one or more substituents independently selected from C₁₋₆ alkyl optionally substituted with one or more substituents independently selected from: 6- to 12-membered bridged heterocycle optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle. In some embodiments, C is selected from

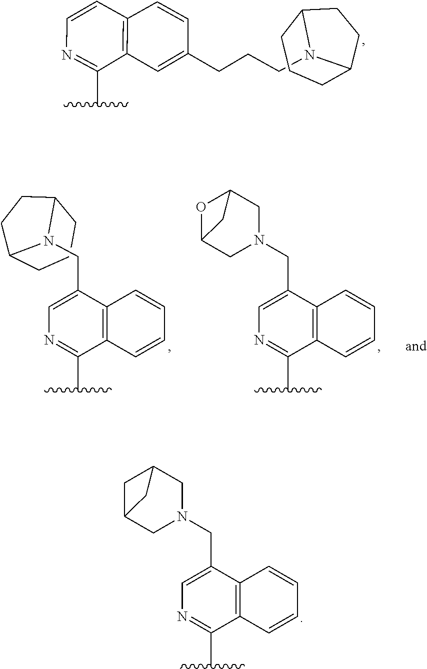 [SMILES: *c1nccc2ccc(CCCN3C4CCCC3CC4)cc12 ; *c1ncc(CN2C(CC)CCC2CC)c2ccccc12 ; *c1ncc(CN2CC3CC(C2)O3)c2ccccc12 ; *c1ncc(CN2CC3CC(C3)C2)c2ccccc12]

In one aspect, provided herein is a compound having the structure of Formula (I-a), or a pharmaceutically acceptable salt or solvate, thereof:

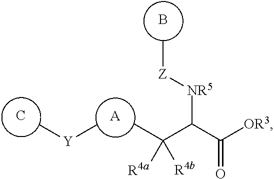

or a pharmaceutically acceptable salt thereof, wherein:
A is selected from:

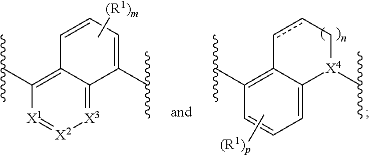

X¹, X², X³, and X⁴ are each independently selected from N and C(R²), wherein at least one of X¹, X², and X³ is C(R²);
[symbol] represents a single bond or a double bond;
m is 0, 1, 2, or 3;
n is 0, 1, or 2;
p is 0, 1, 2, 3, 4, or 5;
each R¹ is independently selected from:
   halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂(R¹¹), —S(O)R¹¹, —S(O)₂R¹¹, —S(O)₂N(R¹¹)₂, —NO₂, and —CN;
   C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN; and
   C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
each R² is independently selected from:
   hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂(R¹²), —S(O)R¹², —S(O)₂R¹², —S(O)₂N(R¹²)₂, —NO₂, and —CN;
   C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
   C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
R^{4a} and R^{4b} are each independently selected from:
   hydrogen, halogen, —OR¹³, —SR¹³, —N(R¹³)₂, —C(O)R¹³, —NO₂, —CN and C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Y is a bond, —O—, —N(R¹⁴)— or —C(R^{14a})(R^{14b})—;
Z is selected from:
   a bond, —C(O)—, —C(R¹⁵)₂—, —C(O) C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—;
   wherein q is selected from 1, 2, and 3;
B is selected from C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —NR¹⁷, —N₃, and —CN; and
      C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN;
C is selected from C₅₋₁₀ carbocycle and 5- to 10-membered heterocycle, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, —NR¹⁸, —N₃, and —CN, and
      C₃₋₆ carbocycle and 3- to 6-membered heterocycle, any of which is optionally substituted with one or more substituents independently selected from: halogen, C₁₋₄ alkyl, and C₁₋₄ haloalkyl; and
   C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, ═O, and CN;
R¹³ is independently selected at each occurrence from hydrogen and C₁₋₆ alkyl;
R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, —O, and —CN;
R^{14a} and R^{14b} are each independently selected from:
   hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, —O, and —CN; or
R^{14a} and R^{14b} are taken together to form a C₃₋₆ carbocycle or a 3- to 6-membered heterocycle, each of which is optionally substituted with one or more substituents independently selected from halogen, —OH, —NH₂, C₁₋₆ alkyl, —C₁₋₆ haloalkyl, —O—C₁₋₆ alkyl, —O—C₁₋₆ haloalkyl, —O, and —CN;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle;
R¹⁶ is selected at each occurrence from hydrogen and C₁₋₆ alkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
   C₃₋₁₀ carbocycle and 3- to 10-membered heterocycle, each of which is optionally substituted with one or more substituents independently selected from: halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and
R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl.

In one aspect, provided herein is a compound having the structure of Formula (II), or a pharmaceutically acceptable salt or solvate, thereof:

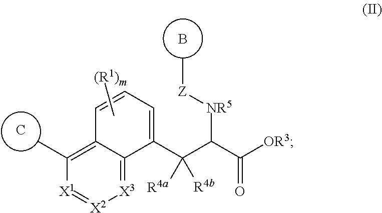

or a pharmaceutically acceptable salt thereof, wherein:
   X¹, X², and X³ are each independently selected from N and C(R²), wherein at least one of
   X¹, X², and X³ is C(R²);
   m is 0, 1, 2, or 3;
   each R¹ is independently selected from:
      halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂(R¹¹), —S(O)R¹¹, —S(O)₂R¹¹, —S(O)₂N(R¹¹)₂, —NO₂, and —CN;
      C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN; and
      C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
   each R² is independently selected from:
      hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂(R¹²), —S(O)R¹², —S(O)₂R¹², —S(O)₂N(R¹²)₂, —NO₂, and —CN;
      C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN;
      and
         C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
R^{4a} and R^{4b} are each independently selected from:
   hydrogen, halogen, —OR¹³, —SR¹³, —N(R¹³)₂, —C(O)R¹³, —NO₂, and —CN; and
      C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Z is selected from:
   a bond, —C(O)—, —C(R¹⁵)₂—, —C(O) C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—;
   wherein q is selected from 1, 2, and 3;
B is selected from C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
      C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, —S, ═NR¹⁷, —N₃, and —CN;
C is selected from C₅₋₁₀ carbocycle and 5- to 10-membered heterocycle, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle, wherein the C₃₋₆ carbocycle and 3- to 6-membered heterocycle are optionally substituted with one or more substituents independently selected from: halogen, C₁₋₄ alkyl, and C₁₋₄ haloalkyl; and
   C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, ═O, and —CN;
R¹³ is independently selected at each occurrence from hydrogen and C₁₋₆ alkyl;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle;
R¹⁶ is selected at each occurrence from hydrogen and C₁₋₆ alkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, —S, ═NR²¹, and —CN; and
   C₃₋₁₀ carbocycle and 3- to 10-membered heterocycle, each of which is optionally substituted with one or more substituents independently selected from:
      halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and
R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl.

In one aspect, provided herein is a compound having the structure of Formula (III), or a pharmaceutically acceptable salt or solvate, thereof:

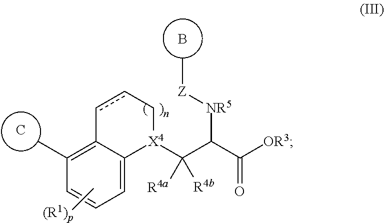

or a pharmaceutically acceptable salt thereof, wherein:
   X⁴ is selected from N and C(R²),
   [symbol] represents a single bond or a double bond;
   n is 0, 1, or 2;
   p is 0, 1, 2, 3, 4 or 5;
   each R¹ is independently selected from:
      halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂(R¹¹), —S(O)R¹¹, —S(O)₂R¹¹, —S(O)₂N(R¹¹)₂, —NO₂, and —CN;
      C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN; and
      C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
   each R² is independently selected from:
      hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂(R¹²), —S(O)R¹², —S(O)₂R¹², —S(O)₂N(R¹²)₂, —NO₂, and —CN;
      C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
      C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
R^{4a} and R^{4b} are each independently selected from:
   hydrogen, halogen, —OR¹³, —SR¹³, —N(R¹³)₂, —C(O)R¹³, —NO₂, and —CN; and
      C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Z is selected from:
   a bond, —C(O)—, —C(R¹⁵)₂—, —C(O) C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—;
   wherein q is selected from 1, 2 and 3;
B is selected from C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle, each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
      C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
      C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN;
C is selected from C₅₋₁₀ carbocycle and 5- to 10-membered heterocycle, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
   C₁₋₆ alkyl, C₂₋₆ alkenyl, and C₂₋₆ alkynyl, each of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle, wherein the C₃₋₆ carbocycle and 3- to 6-membered heterocycle are optionally substituted with one or more substituents independently selected from: halogen, C₁₋₄ alkyl, and C₁₋₄ haloalkyl; and
   C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, —O, and —CN;
R¹³ is independently selected at each occurrence from hydrogen and C₁₋₆ alkyl;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle;
R¹⁶ is selected at each occurrence from hydrogen and C₁₋₆ alkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
   C₃₋₁₀ carbocycle and 3- to 10-membered heterocycle, each of which is optionally substituted with one or more substituents independently selected from:
      halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and
R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl.

In some embodiments, for the compound or salt of Formulas (I), (I-a), (II) or (III), m is 0, 1, 2, or 3. In some embodiments, m is 0, 1, or 2. In some embodiments, m is 0, or 1. In some embodiments, m is 1, 2, or 3. In some embodiments, m is 1, or 2. In some embodiments, m is 0. In some embodiments, m is 1. In some embodiments, m is 2. In some embodiments, m is 3.

In some embodiments, for the compound or salt of Formulas (I), (I-a), (II) or (III), n is 0, 1, or 2. In some embodiments, n is 0 or 1. In some embodiments, n is 1 or 2. In some embodiments, n is 0. In some embodiments, n is 1. In some embodiments, n is 2.

In some embodiments, for the compound or salt of Formulas (I), (I-a), (II) or (III), p is 0, 1, 2, 3, or 4. In some embodiments, p is 0, 1, 2, or 3. In some embodiments, p is 0, 1, or 2. In some embodiments, p is 0, or 1. In some embodiments, p is 1, 2, or 3. In some embodiments, p is 1 or 2. In some embodiments, p is 2 or 3. In some embodiments, p is 0. In some embodiments, p is 1. In some embodiments, p is 2. In some embodiments, p is 3. In some embodiments, p is 4. In some embodiments, p is 5.

In some embodiments, for the compound or salt of Formulas (I), A is selected from

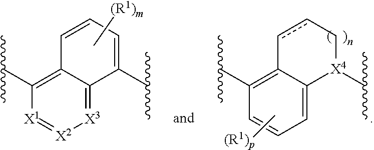

In some embodiments, A is

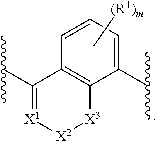

In some embodiments, A is selected from

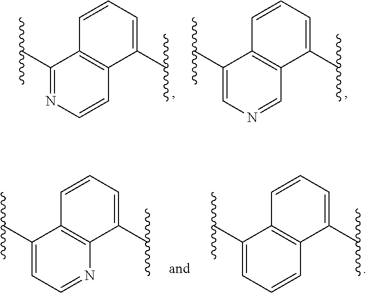

In some embodiments, A is selected from

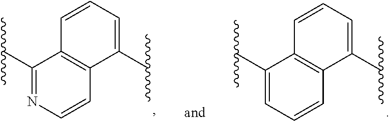 [SMILES: *c1cccc2c(*)nccc12 ; *c1cccc2c(*)cccc12]

In some embodiments, A is

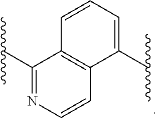 [SMILES: *c1cccc2c(*)nccc12]

In some embodiments, A is

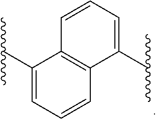 [SMILES: *c1cccc2c(*)cccc12]

In some embodiments, for the compound or salt of Formula (I), A is

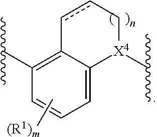

In some embodiments, A is

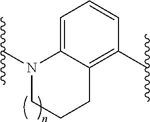

and n is 0 or 1. In some embodiments, A is

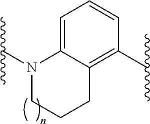

and n is 0. In some embodiments, A is

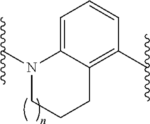

and n is 1. In some embodiments, A is

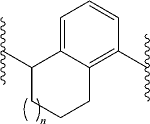

and n is 0 or 1. In some embodiments, A is

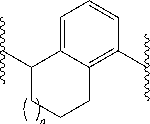

and n is 0. In some embodiments, A is

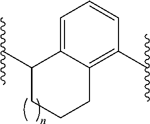

and n is 1.

In some embodiments, for the compound or salt of Formulas (I) or (II), X¹, X², and X³ are each independently selected from N and C(R²), wherein at least one of X¹, X², and X³ is C(R²). In some embodiments, X¹ is N, and X² and X³ are each independently selected from N and C(R²), wherein at least one of X² and X³ is C(R²). In some embodiments, X² is N, and X¹ and X³ are each independently selected from N and C(R²), wherein at least one of X¹ and X³ is C(R²). In some embodiments, X³ is N, and X² and X³ are each independently selected from N and C(R²), wherein at least one of X² and X³ is C(R²). In some embodiments, X¹ is C(R²), and X² and X³ are each independently selected from N and C(R²). In some embodiments, X² is C(R²), and X¹ and X³ are each independently selected from N and C(R²). In some embodiments, X³ is C(R²), and X² and X³ are each independently selected from N and C(R²).

In some embodiments, for the compound or salt of Formulas (I) or (III), X⁴ is selected from Nand C(R²). In some embodiments, X⁴ is C(R²). In some embodiments, X⁴ is N. In some embodiments, [symbol] represents a single bond.

In some embodiments, for the compound or salt of Formulas (I), Y is a bond, —O—, —N(R¹⁴)— or —C(R^{14a})(R^{14b})—. In some embodiments, Y is a bond, —N(R¹⁴)—, or —C(R^{14a})(R^{14b})—. In some embodiments, Y is a bond, —O—, or —C(R^{14a})(R^{14b})—. In some embodiments, Y is a bond or —C(R^{14a})(R^{14b})—. In some embodiments, Y is a bond.

In some embodiments, for the compound or salt of Formulas (I), (I-a), (II) or (III), each R¹ is independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂(R¹¹), —S(O)R¹¹, —S(O)₂R¹¹, —S(O)₂N(R¹¹)₂, —NO₂, and —CN;
C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN; and
C₃₋₆ carbocycle and 3- to 6-membered heterocycle, each of which is optionally substituted with one or more substituents independently selected from halogen, C₁₋₄ alkyl, C₁₋₄haloalkyl, —OR¹¹, —SR¹¹, —N(R¹¹)₂, ═O, ═S, and —CN.

In some embodiments, for the compound or salt of Formulas (I), (I-a), (II) or (III), each R¹ is independently selected from:
halogen, C₁₋₆ alkyl, C₂₋₆ alkenyl, C₂₋₆ alkynyl, C₁₋₆ haloalkyl, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —NO₂ and —CN; and
C₃₋₆ carbocycle and 3- to 6-membered heterocycle, each of which is optionally substituted with one or more substituents independently selected from halogen, C₁₋₄ alkyl, C₁₋₄haloalkyl, —OR¹¹, —SR¹¹, —N(R¹¹)₂, ═O, ═S, and —CN.

In some embodiments, for the compound or salt of Formulas (I), (I-a), (II) or (III), each R¹ is independently selected from: halogen, C₁₋₆ alkyl, C₂₋₆ alkenyl, C₂₋₆ alkynyl, C₁₋₆ haloalkyl, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —NO₂, and —CN. In some embodiments, each R¹ is independently selected from: halogen, C₁₋₆ alkyl, C₂₋₆ alkenyl, C₂₋₆ alkynyl, C₁₋₆ haloalkyl, —OR¹¹, —N(R¹¹)₂, —C(O)R¹¹, —C(O)OR¹¹, —OC(O)R¹¹, —C(O)N(R¹¹)₂, —N(R¹¹)C(O)R¹¹, —NO₂, and —CN. In some embodiments, each R¹ is independently selected from: halogen, C₁₋₆ alkyl, C₂₋₆ alkenyl, C₂₋₆ alkynyl, C₁₋₆ haloalkyl, —OR¹¹, —N(R¹¹)₂, —NO₂, and —CN. In some embodiments, each R¹ is independently selected from: halogen, C₁₋₆ alkyl, C₂₋₆ alkenyl, C₂₋₆ alkynyl, C₁₋₆ haloalkyl, —OR¹¹, and —CN. In some embodiments, each R¹ is independently selected from: halogen, C₁₋₆ alkyl, and C₁₋₆ haloalkyl. In some embodiments, each R¹ is independently selected from halogen and C₁₋₆ alkyl. In some embodiments, each R¹ is independently selected from halogen. In some embodiments, each R¹ is independently selected from C₁₋₆ alkyl.

In some embodiments, for the compound or salt of Formulas (I), (I-a), (II) or (III), each R¹ is independently selected from C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN. In one aspect, provided herein is a compound having the structure of Formulas (I), (I-a), (II) or (III), or a pharmaceutically acceptable salt or solvate thereof, each R¹ is independently selected from C₃₋₆ carbocycle and 3- to 6-membered heterocycle, each of which is optionally substituted with one or more substituents independently selected from halogen, C₁₋₄ alkyl, C₁₋₄haloalkyl, —OR¹¹, —N(R¹¹)₂, ═O, and —CN. In some embodiments, each R¹ is independently selected from C₃₋₆ carbocycle and 3- to 4-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN. In some embodiments, each R¹ is independently selected from C₃₋₄ carbocycle and 3- to 4-membered heterocycle, each of which is optionally substituted with one or more substituents independently selected from halogen, C₁₋₄ alkyl, C₁₋₄haloalkyl, —OR¹¹, —SR¹¹, —N(R¹¹)₂, ═O, ═S, and —CN. In some embodiments, each R¹ is independently selected from C₃₋₄ carbocycle and 3- to 4-membered heterocycle, each of which is optionally substituted with one or more substituents independently selected from halogen, C₁₋₄ alkyl, C₁₋₄haloalkyl, —OR¹¹, —N(R¹¹)₂, —O, and —CN. In some embodiments, each R¹ is independently selected from C₃₋₄ carbocycle and 3- to 4-membered heterocycle, each of which is optionally substituted with one or more substituents independently selected from halogen, C₁₋₄ alkyl, and C₁₋₄ haloalkyl. In some embodiments, each R¹ is independently selected from C₃₋₄ carbocycle and 3- to 4-membered heterocycle, each of which is optionally substituted with one or more substituents independently selected from halogen and C₁₋₄alkyl.

In some embodiments, for the compound or salt of Formulas (I), (I-a), (II) or (III), each R² is independently selected from:
hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂(R¹²), —S(O)R¹², —S(O)₂R¹², —S(O)₂N(R¹²)₂, —NO₂, and —CN;
C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN.

In some embodiments, for the compound or salt of Formulas (I), (I-a), (II) or (III), each R² is independently selected from: hydrogen, halogen, C₁₋₆ alkyl, C₂₋₆ alkenyl, C₂₋₆ alkynyl, C₁₋₆ haloalkyl, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —NO₂, and —CN. In some embodiments, each R² is independently selected from: halogen, C₁₋₆ alkyl, C₂₋₆ alkenyl, C₂₋₆ alkynyl, C₁₋₆ haloalkyl, —OR¹², —N(R¹²)₂, —C(O)R¹², —C(O)OR¹², —OC(O)R¹², —C(O)N(R¹²)₂, —N(R¹²)C(O)R¹², —NO₂, and —CN. In some embodiments, each R² is independently selected from: halogen, C₁₋₆ alkyl, C₂₋₆ alkenyl, C₂₋₆ alkynyl, C₁₋₆ haloalkyl, —OR¹², —N(R¹²)₂, —NO₂, and —CN. In some embodiments, each R² is independently selected from: halogen, C₁₋₆ alkyl, C₂₋₆ alkenyl, C₂₋₆ alkynyl, C₁₋₆ haloalkyl, —OR¹², and —CN. In some embodiments, each R² is independently selected from: halogen, C₁₋₆ alkyl, and C₁₋₆ haloalkyl. In some embodiments, each R² is independently selected from halogen and C₁₋₆ alkyl. In some embodiments, each R² is independently selected from halogen. In some embodiments, each R² is independently selected from C₁₋₆ alkyl.

In some embodiments, for the compound or salt of Formulas (I), (I-a), (II) or (III), each R² is independently selected from C₃₋₆ carbocycle and 3- to 6-membered heterocycle, each of which is optionally substituted with one or more substituents independently selected from halogen, C₁₋₄ alkyl, C₁₋₄haloalkyl, —OR¹², —SR¹², —N(R¹²)₂, ═O, ═S, and —CN. In one aspect, provided herein is a compound having the structure of Formulas (I), (I-a), (II) or (III), or a pharmaceutically acceptable salt or solvate thereof, each R² is independently selected from C₃₋₆ carbocycle and 3- to 6-membered heterocycle, each of which is optionally substituted with one or more substituents independently selected from halogen, C₁₋₄ alkyl, C₁₋₄haloalkyl, —OR¹², —N(R¹²)₂, —O, and —CN. In some embodiments, each R² is independently selected from C₃₋₄ carbocycle and 3- to 4-membered heterocycle, each of which is optionally substituted with one or more substituents independently selected from halogen, C₁₋₄ alkyl, C₁₋₄haloalkyl, —OR¹², —SR¹², —N(R¹²)₂, ═O, ═S, and —CN. In some embodiments, each R² is independently selected from C₃₋₄ carbocycle and 3- to 4-membered heterocycle, each of which is optionally substituted with one or more substituents independently selected from halogen, C₁₋₄ alkyl, C₁₋₄haloalkyl, —OR¹², —SR¹², —N(R¹²)₂, ═O, ═S, and —CN. In some embodiments, each R² is independently selected from C₃₋₄ carbocycle and 3- to 4-membered heterocycle, each of which is optionally substituted with one or more substituents independently selected from halogen, C₁₋₄ alkyl, C₁₋₄ haloalkyl, —OR¹², —N(R¹²)₂, ═O, and —CN. In some embodiments, each R² is independently selected from C₃₋₄ carbocycle and 3- to 4-membered heterocycle, each of which is optionally substituted with one or more substituents independently selected from halogen, C₁₋₄alkyl, and C₁₋₄ haloalkyl. In some embodiments, each R² is independently selected from C₃₋₄ carbocycle and 3- to 4-membered heterocycle, each of which is optionally substituted with one or more substituents independently selected from halogen and C₁₋₄alkyl.

In some embodiments, for the compound or salt of Formulas (I), (I-a), (II) or (III), R³ is independently selected from hydrogen and C₁₋₆ alkyl. In some embodiments, R³ is independently selected from hydrogen, methyl, ethyl, propyl and butyl. In some embodiments, R³ is independently selected from hydrogen, methyl and ethyl. In some embodiments, R³ is independently selected from hydrogen and methyl. In some embodiments, R³ is selected from hydrogen. In some embodiments, R³ is selected from methyl.

In some embodiments, for the compound or salt of Formulas (I), (I-a), (II) or (III), R^{4a} and R^{4b} are each independently selected from hydrogen, halogen, —OR¹³, —SR¹³, —N(R¹³)₂, —C(O)R¹³, —NO₂, —CN, and C₁₋₆ alkyl optionally substituted with one or more substituents independently selected from halogen, —OR¹³, —SR¹³, —N(R¹³)₂, —C(O)R¹³, —NO₂, and —CN. In some embodiments, R^{4a} and R^{4b} are each independently selected from hydrogen, halogen, —OR¹³, —SR¹³, —N(R¹³)₂, —C(O)R¹³, —NO₂, —CN, and C₁₋₆ alkyl. In some embodiments, R^{4a} and R^{4b} are each independently selected from hydrogen, halogen, —OR¹³, —N(R¹³)₂, —NO₂, —CN, and C₁₋₆ alkyl. In some embodiments, R^{4a} and R^{4b} are each independently selected from hydrogen, halogen, —OR¹³, —N(R¹³)₂, —CN, and C₁₋₆ alkyl. In some embodiments, R^{4a} and R^{4b} are each independently selected from hydrogen, halogen, —CN, and C₁₋₆ alkyl. In some embodiments, R^{4a} and R^{4b} are each independently selected from hydrogen, halogen, and C₁₋₆ alkyl. In some embodiments, R^{4a} and R^{4b} are each independently selected from hydrogen, halogen, C₁₋₃ alkyl, and C₁₋₃ haloalkyl. In some embodiments, R^{4a} and R^{4b} are each independently selected from hydrogen and halogen. In some embodiments, R^{4a} and R^{4b} are each hydrogen.

In some embodiments, for the compound or salt of Formulas (I), (I-a), (II) or (III), R⁵ is independently selected from hydrogen and C₁₋₆ alkyl. In some embodiments, R⁵ is independently selected from hydrogen, methyl, and ethyl. In some embodiments, R⁵ is hydrogen. In some embodiments, R⁵ is methyl.

In some embodiments, for the compound or salt of Formulas (I), (I-a), (II) or (III), Z is selected from a bond, —C(O)—, —C(R¹⁵)₂—, —C(O) C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—. In some embodiments, Z is selected from a bond, —C(O)—, —C(R¹⁵)₂—, —C(O) C(R¹⁵)₂—, —C(O) [C(R¹⁵)₂]_{q}S—, —C(═N—CN)—, —C(O)O—, —C(O)N(R¹⁶)—, —C(S)N(R¹⁶)—, and —S(O)₂—. In some embodiments, Z is selected from a bond, —C(O)—, —C(R¹⁵)₂—, —C(O) C(R¹⁵)₂—, —C(O) [C(R¹⁵)₂]_{q}S—, —C(═N—CN)—, —C(O)O—, and —S(O)₂—. In some embodiments, Z is selected from a bond, —C(O)—, —C(R¹⁵)₂—, —C(O) C(R¹⁵)₂—, —C(O) [C(R¹⁵)₂]_{q}S—, and —C(═N—CN)—. In some embodiments, Z is selected from —C(O)—, —C(R¹⁵)₂—, —C(O) C(R¹⁵)₂—, —C(O) [C(R¹⁵)₂]_{q}S—, and —C(═N—CN)—. In some embodiments, Z is —C(O)—. In some embodiments, Z is —C(R¹⁵)₂—. In some embodiments, Z is —C(O) C(R¹⁵)₂—. In some embodiments, Z is —C(O) [C(R¹⁵)₂]_{q}S—. In some embodiments, Z is —C(═N—CN)—.

In some embodiments, for the compound or salt of Formulas (I), (I-a), (II) or (III), q is selected from 1, 2, and 3. In some embodiments, q is selected from 1 and 2. In some embodiments, q is 1. In some embodiments, q is 2.

In some embodiments, for the compound or salt of Formulas (I), (I-a), (II) or (III), R¹¹ and R¹² are each independently selected at each occurrence from hydrogen and C₁₋₆ alkyl optionally substituted with one or more substituents independently selected from: halogen, —OH, —NH₂, —O—C₁₋₆ alkyl, —O—C₁₋₆ haloalkyl, ═O, and —CN. In some embodiments, R¹¹ and R¹² are each independently selected at each occurrence from hydrogen and C₁₋₆ alkyl optionally substituted with one or more substituents independently selected from: halogen, —OH, —NH₂, ═O, and —CN. In some embodiments, R¹¹ and R¹² are each independently selected at each occurrence from hydrogen and C₁₋₆ alkyl optionally substituted with one or more substituents independently selected from: halogen, —OH, —NH₂, and ═O. In some embodiments, R¹¹ and R¹² are each independently selected at each occurrence from hydrogen and C₁₋₆ alkyl optionally substituted with one or more substituents independently selected from: halogen, —OH, and ═O. In some embodiments, R¹¹ and R¹² are each independently selected at each occurrence from hydrogen and C₁₋₆ alkyl optionally substituted with one or more substituents independently selected from: halogen and ═O. In some embodiments, R¹¹ and R¹² are each independently selected at each occurrence from hydrogen, C₁₋₆ alkyl, and C₁₋₆ haloalkyl. In some embodiments, R¹¹ and R¹² are each independently selected at each occurrence from hydrogen and C₁₋₆ alkyl. In some embodiments, each occurrence of R¹¹ and R¹² is independently hydrogen.

In some embodiments, for the compound or salt of Formulas (I), (I-a), (II) or (III), R¹³ is independently selected at each occurrence from hydrogen and C₁₋₆ alkyl. In some embodiments, R¹³ is independently selected at each occurrence from hydrogen and C₁₋₄ alkyl. In some embodiments, each R¹³ is hydrogen.

In some embodiments, for the compound or salt of Formulas (I), (I-a), (II) or (III), R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, —O, and —CN. In some embodiments, R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN. In some embodiments, R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —O—C₁₋₆ alkyl, ═O, and —CN. In some embodiments, R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —O—C₁₋₆ alkyl, and ═O. In some embodiments, R¹⁴ is selected from hydrogen and C₁₋₆ alkyl optionally substituted with one or more substituents independently selected from halogen, —OH, —O—C₁₋₆ alkyl, and ═O. In some embodiments, R¹⁴ is selected from hydrogen, C₁₋₆ alkyl, and C₁₋₆ haloalkyl. In some embodiments, R¹⁴ is selected from hydrogen.

In some embodiments, for the compound or salt of Formulas (I), (I-a), (II) or (III), R^{14a} and R^{14b} are each independently selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN; or R^{14a} and R^{14b} are taken together to form a C₃₋₆ carbocycle or a 3- to 6-membered heterocycle, each of which is optionally substituted with one or more substituents independently selected from halogen, —OH, C₁₋₆ alkyl, —C₁₋₆ haloalkyl, —O—C₁₋₆ alkyl, —O—C₁₋₆ haloalkyl, ═O, and —CN. In some embodiments, R^{14a} and R^{14b} are each independently selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —O—C₁₋₆ alkyl, ═O, and —CN; or R^{14a} and R^{14b} are taken together to form a C₃₋₆ carbocycle or a 3- to 6-membered heterocycle, each of which is optionally substituted with one or more substituents independently selected from halogen, —OH, C₁₋₆ alkyl, —C₁₋₆ haloalkyl, —O—C₁₋₆ alkyl, —O—C₁₋₆ haloalkyl, ═O, and —CN. In some embodiments, R^{14a} and R^{14b} are each independently selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, and ═O; or R^{14a} and R^{14b} are taken together to form a C₃₋₆ carbocycle or a 3- to 6-membered heterocycle, each of which is optionally substituted with one or more substituents independently selected from halogen, —OH, C₁₋₆ alkyl, —C₁₋₆ haloalkyl, and ═O.

In some embodiments, for the compound or salt of Formulas (I), (I-a), (II) or (III), R^{14a} and R^{14b} are each independently selected from:
hydrogen and C₁₋₆ alkyl optionally substituted with one or more substituents independently selected from halogen, —OH, —NH₂, —O—C₁₋₆ alkyl, —O—C₁₋₆ haloalkyl, ═O, and —CN; or
R^{14a} and R^{14b} are taken together to form a C₃₋₆ carbocycle or a 3- to 6-membered heterocycle, each of which is optionally substituted with one or more substituents independently selected from halogen, —OH, —NH₂, C₁₋₆ alkyl, —C₁₋₆ haloalkyl, —O—C₁₋₆ alkyl, —O—C₁₋₆ haloalkyl, ═O, and —CN.

In some embodiments, for the compound or salt of Formulas (I), (I-a), (II) or (III), R^{14a} and R^{14b} are each independently selected from:
hydrogen and C₁₋₆ alkyl optionally substituted with one or more substituents independently selected from halogen, —OH, —NH₂, —O—C₁₋₆ alkyl, —O—C₁₋₆ haloalkyl, ═O, and —CN; or
R^{14a} and R^{14b} are taken together to form a C₃₋₆ carbocycle or a 3- to 6-membered heterocycle.

In some embodiments, for the compound or salt of Formulas (I), (I-a), (II) or (III), R^{14a} and R^{14b} are each independently selected from: hydrogen and C₁₋₆ alkyl optionally substituted with one or more substituents independently selected from halogen, —OH, —NH₂, —O—C₁₋₆ alkyl, —O—C₁₋₆ haloalkyl, ═O, and —CN. In some embodiments, R^{14a} and R^{14b} are each independently selected from: hydrogen and C₁₋₆ alkyl optionally substituted with one or more substituents independently selected from halogen, —OH, —O—C₁₋₆ alkyl, —O—C₁₋₆ haloalkyl, and ═O. In some embodiments, R^{14a} and R^{14b} are each independently selected from: hydrogen and C₁₋₆ alkyl optionally substituted with one or more substituents independently selected from halogen and ═O. In some embodiments, R^{14a} and R^{14b} are each independently selected from: hydrogen and C₁₋₆ alkyl. In some embodiments, R^{14a} and R^{14b} are each independently hydrogen.

In some embodiments, for the compound or salt of Formulas (I), (I-a), (II) or (III), R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle. In some embodiments, R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle. In some embodiments, R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle. In some embodiments, R¹⁵ is independently selected at each occurrence from hydrogen, halogen, ═O, —CN, C₁₋₆ alkyl, C₁₋₆ haloalkyl, and 3- to 6-membered heterocycle. In some embodiments, R¹⁵ is independently selected at each occurrence from hydrogen, halogen, ═O, C₁₋₆ alkyl, C₁₋₆ haloalkyl, and 3- to 6-membered heterocycle. In some embodiments, R¹⁵ is independently selected at each occurrence from hydrogen, halogen, C₁₋₆ alkyl, C₁₋₆ haloalkyl, and 3- to 6-membered heterocycle. In some embodiments, R¹⁵ is independently selected at each occurrence from hydrogen, halogen, C₁₋₆ alkyl, and 3- to 6-membered heterocycle. In some embodiments, R¹⁵ is independently selected at each occurrence from hydrogen, halogen, and C₁₋₆ alkyl. In some embodiments, R¹⁵ is independently selected at each occurrence from hydrogen, and C₁₋₆ alkyl. In some embodiments, R¹⁵ is independently selected at each occurrence from hydrogen, and halogen. In some embodiments, each R¹⁵ is hydrogen.

In some embodiments, for the compound or salt of Formulas (I), (I-a), (II) or (III), R¹⁶ is selected at each occurrence from hydrogen and C₁₋₆ alkyl. In some embodiments, R¹⁶ is selected at each occurrence from hydrogen and C₁₋₄ alkyl. In some embodiments, R¹⁶ is selected at each occurrence from hydrogen and C₁₋₄ alkyl. In some embodiments, each R¹⁶ is hydrogen.

In some embodiments, for the compound or salt of Formulas (I), (I-a), (II) or (III), B is selected from C₃₋₁₀ carbocycle and 3- to 10-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN.

In some embodiments, for the compound or salt of Formulas (I), (I-a), (II) or (III), B is selected from C₃₋₁₀ carbocycle and 3- to 10-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, —S, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, ═O, and —CN.

In some embodiments, for the compound or salt of Formulas (I), (I-a), (II) or (III), B is selected from C₃₋₁₀ carbocycle and 3- to 10-membered heterocycle, each of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, ═O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, —O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from C₃₋₁₀ carbocycle and 3- to 10-membered heterocycle, each of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), R¹⁷ is independently selected at each occurrence from:
hydrogen;
C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
C₃₋₁₀ carbocycle and 3- to 10-membered heterocycle, each of which is optionally substituted with one or more substituents independently selected from:
   halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle.

In some embodiments, for the compound or salt of Formulas (I), (I-a), (II) or (III), R¹⁷ is independently selected at each occurrence from:
hydrogen;
C₁₋₆ alkyl optionally substituted with one or more substituents independently selected from: halogen, —OR²¹, —N(R²¹)₂, —C(O)R²¹, —C(O)OR²¹, —OC(O)R²¹, —C(O)N(R²¹)₂, —N(R²¹)C(O)R²¹, —O, and —CN; and
C₃₋₁₀ carbocycle and 3- to 10-membered heterocycle, each of which is optionally substituted with one or more substituents independently selected from: halogen, C₁₋₆ alkyl, C₁₋₆ haloalkyl —OR²¹, —N(R²¹)₂, —C(O)R²¹, —C(O)OR²¹, —OC(O)R²¹, —C(O)N(R²¹)₂, —N(R²¹)C(O)R²¹, —N(R²¹)S(O)₂(R²¹), —S(O)R²¹, —S(O)₂R²¹, —S(O)₂N(R²¹)₂, ═O, —CN, C₃₋₆ carbocycle, and 3- to 6-membered heterocycle.

In some embodiments, for the compound or salt of Formulas (I), (I-a), (II) or (III), R¹⁷ is independently selected at each occurrence from:
hydrogen;
C₁₋₆ alkyl optionally substituted with one or more substituents independently selected from: halogen, —OR²¹, —N(R²¹)₂, —C(O)N(R²¹)₂, —N(R²¹)C(O)R²¹, ═O, and —CN; and
C₃₋₁₀ carbocycle and 3- to 10-membered heterocycle, each of which is optionally substituted with one or more substituents independently selected from: halogen, C₁₋₆ alkyl, C₁₋₆ haloalkyl —OR²¹, —N(R²¹)₂, —C(O)R²¹, —C(O)N(R²¹)₂, —N(R²¹)C(O)R²¹, ═O, C₃₋₆ carbocycle, and 3- to 6-membered heterocycle.

In some embodiments, for the compound or salt of Formulas (I), (I-a), (II) or (III), R¹⁷ is independently selected at each occurrence from:
hydrogen;
C₁₋₆ alkyl optionally substituted with one or more substituents independently selected from halogen, —OR²¹, —SR²¹, —N(R²¹)₂, and —CN; and
C₃₋₁₀ carbocycle and 3- to 10-membered heterocycle, each of which is optionally substituted with one or more substituents independently selected from: halogen, C₁₋₆ alkyl, C₁₋₆ haloalkyl, —OC(O)N(R²¹)₂, and C₃₋₆ carbocycle.

In some embodiments, for the compound or salt of Formulas (I), (I-a), (II) or (III), R¹⁷ is independently selected at each occurrence from: hydrogen; C₁₋₆ alkyl optionally substituted with one or more substituents independently selected from halogen and —N(R²¹)₂; and C₃₋₁₀ carbocycle and 3- to 10-membered heterocycle, each of which is optionally substituted with one or more substituents independently selected from —OC(O)N(R²¹)₂, and C₃₋₆ carbocycle. In some embodiments, R¹⁷ is independently selected at each occurrence from: hydrogen and C₁₋₆ alkyl optionally substituted with one or more substituents independently selected from halogen, —OR²¹, —SR²¹, —N(R²¹)₂, and —CN. In some embodiments, R¹⁷ is independently selected at each occurrence from: hydrogen and C₁₋₆ alkyl optionally substituted with one or more substituents independently selected from halogen, —N(R²¹)₂, and —CN. In some embodiments, R¹⁷ is independently selected at each occurrence from: hydrogen and C₁₋₆ alkyl optionally substituted with one or more substituents independently selected from halogen and —N(R²¹)₂. In some embodiments, R¹⁷ is independently selected at each occurrence from: hydrogen; and C₃₋₁₀ carbocycle and 3- to 10-membered heterocycle, each of which is optionally substituted with one or more substituents independently selected from: halogen, C₁₋₆ alkyl, C₁₋₆ haloalkyl, —OC(O)N(R²¹)₂, and C₃₋₆ carbocycle. In some embodiments, R¹⁷ is independently selected at each occurrence from: hydrogen; and C₃₋₁₀ carbocycle and 3- to 10-membered heterocycle, each of which is optionally substituted with one or more substituents independently selected from: halogen, C₁₋₆ alkyl, —OC(O)N(R²¹)₂, and C₃₋₆ carbocycle. In some embodiments, R¹⁷ is independently selected at each occurrence from: hydrogen; and C₃₋₁₀ carbocycle and 3- to 10-membered heterocycle, each of which is optionally substituted with one or more substituents independently selected from —OC(O)N(R²¹)₂, and C₃₋₆ carbocycle.

In some embodiments, for the compound or salt of Formulas (I), (I-a), (II) or (III), R¹⁸ and R²¹ are each independently selected at each occurrence from rom hydrogen, C₁₋₆ alkyl, C₁₋₆ haloalkyl, C₁₋₆ hydroxyalkyl, and C₁₋₆ aminoalkyl. In some embodiments, for the compound or salt of Formulas (I), (I-a), (II) or (III), R¹⁸ and R²¹ are each independently selected at each occurrence from hydrogen, C₁₋₆ alkyl, C₁₋₆ haloalkyl, and C₁₋₆ hydroxyalkyl. In some embodiments, R¹⁸ and R²¹ are each independently selected at each occurrence from hydrogen, C₁₋₆ alkyl, and C₁₋₆ haloalkyl. In some embodiments, R¹⁸ and R²¹ are each independently selected at each occurrence from hydrogen and C₁₋₆ alkyl.

In some embodiments, for the compound or salt of Formulas (I), (I-a), (II) or (III), R²¹ is independently selected at each occurrence from hydrogen, C₁₋₆ alkyl, C₁₋₆ haloalkyl, and C₁₋₆ hydroxyalkyl. In some embodiments, R²¹ is independently selected at each occurrence from hydrogen, C₁₋₆ alkyl, and C₁₋₆ haloalkyl. In some embodiments, R²¹ is independently selected at each occurrence from hydrogen and C₁₋₆ alkyl. In some embodiments, R²¹ is independently selected at each occurrence from C₁₋₆ alkyl. In some embodiments, each R²¹ is hydrogen.

In some embodiments, for the compound or salt of Formulas (I), (I-a), (II) or (III), B is selected from C₃₋₁₀ carbocycle and 3- to 10-membered heterocycle, each of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

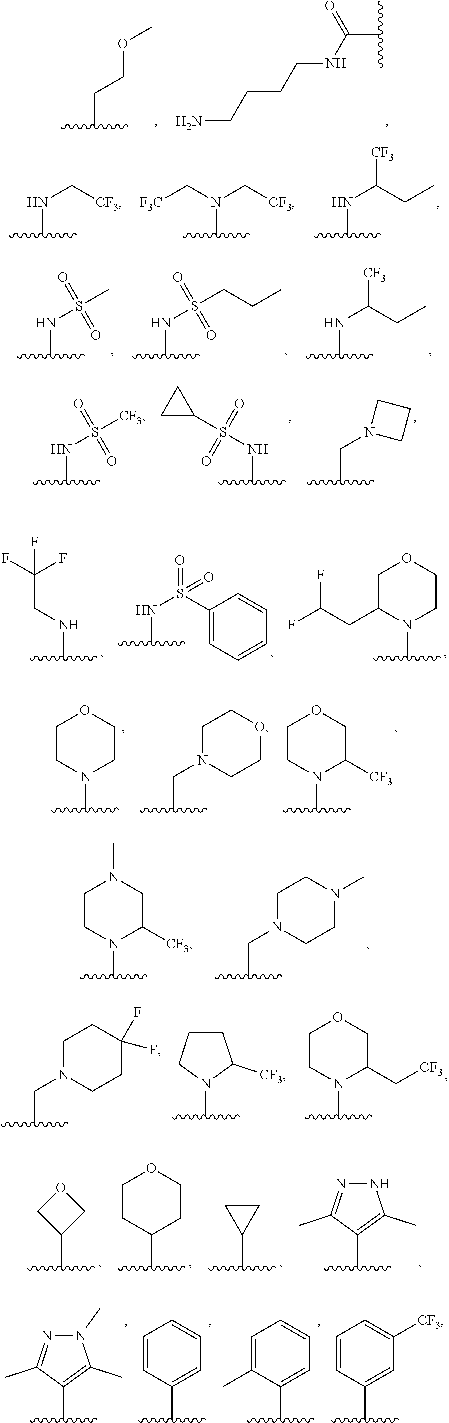

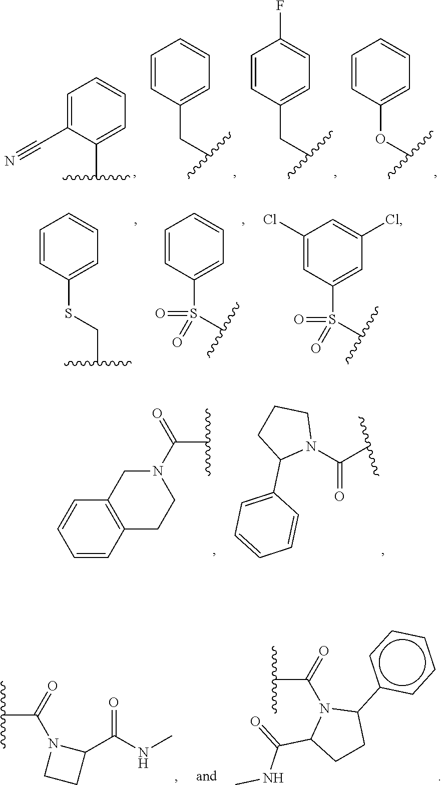

In some embodiments, for the compound or salt of Formulas (I), (I-a), (II) or (III), B is selected from C₃₋₁₀ carbocycle and 3- to 10-membered heterocycle, each of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

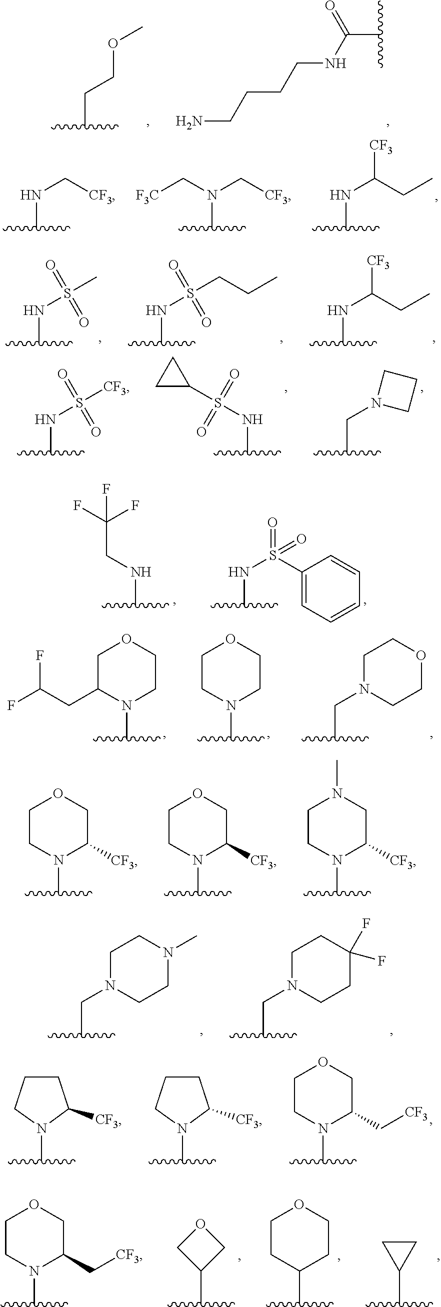

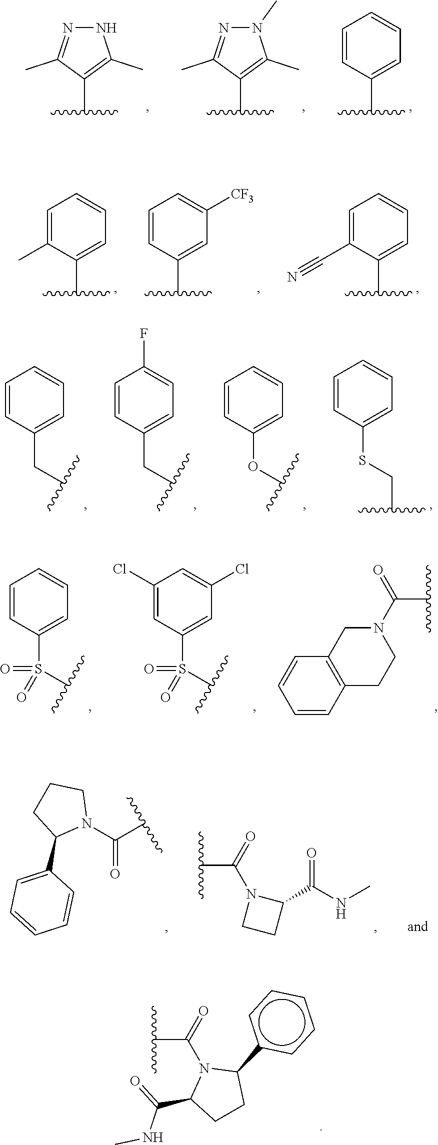

In some embodiments, for the compound or salt of Formulas (I), (I-a), (II) or (III), B is selected from: phenyl, naphthyl, C₃₋₁₀ cycloalkyl, 5- to 6-membered monocyclic heterocycle, and 7- to 10-membered bicyclic heterocycle, any of which is optionally substituted. In some embodiments, B is selected from: phenyl, C₃₋₇ monocyclic cycloalkyl, and 5- to 6-membered monocyclic heterocycle, any of which is optionally substituted. In some embodiments, B is selected from: naphthyl, C₇-10 bicyclic cycloalkyl, and 7- to 10-membered bicyclic heterocycle, any of which is optionally substituted. In some embodiments, B is optionally substituted phenyl. In some embodiments, B is optionally substituted naphthyl. In some embodiments, B is optionally substituted C₃₋₁₀ cycloalkyl. In some embodiments, B is optionally substituted 5- to 6-membered monocyclic heterocycle. In some embodiments, B is optionally substituted 7- to 10-membered bicyclic heterocycle.

In some embodiments, for the compound or salt of Formulas (I), (I-a), (II) or (III), B is selected from: phenyl, naphthyl, cyclopropyl, cyclohexyl, bicyclo[2.2.2]octane, adamantane, thiazole, pyrrole, pyrazole, pyridine, pyrimidine, pyrazine, indole, indazole, benzimidazole, indoline, azaindole, azaindoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted. In some embodiments, B is selected from: phenyl, naphthyl, cyclopropyl, cyclohexyl, bicyclo[2.2.2]octane, adamantane, thiazole, pyrazole, pyridine, indole, indazole, benzimidazole, indoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted. In some embodiments, B is selected from: phenyl, cyclohexyl, pyrazole, pyridine, indole, indazole, benzimidazole, indoline, 1,3-dihydrobenzo[c]isothiazole, pyrrolidine, piperidine, pyrrolidine, and piperidine, any of which is optionally substituted. In some embodiments, B is selected from: phenyl, indole, indazole, benzimidazole, and 1,3-dihydrobenzo[c]isothiazole, any of which is optionally substituted. In some embodiments, B is optionally substituted phenyl.

In some embodiments, for the compound or salt of Formulas (I), (I-a), (II) or (III), B is selected from: phenyl, cyclopropyl, cyclohexyl, thiazole, pyrrole, pyrazole, pyridine, pyrimidine, pyrazine, indole, indazole, tetrahydropyran, azetidine, pyrrolidine, piperidine, and azepane, any of which is optionally substituted. In some embodiments, B is selected from: phenyl, cyclopropyl, cyclohexyl, thiazole, pyrazole, pyridine, indole, indazole, tetrahydropyran, azetidine, pyrrolidine, piperidine, and azepane, any of which is optionally substituted. In some embodiments, B is selected from: naphthyl, bicyclo[2.2.2]octane, adamantane, indole, indazole, benzimidazole, indoline, azaindole, azaindoline, 1,3-dihydrobenzo[c]isothiazole, quinuclidine, and 8-azabicyclo[3.2.1]octane, any of which is optionally substituted. In some embodiments, B is selected from: naphthyl, bicyclo[2.2.2]octane, adamantane, indole, indazole, benzimidazole, indoline, 1,3-dihydrobenzo[c]isothiazole, quinuclidine, and 8-azabicyclo[3.2.1]octane, any of which is optionally substituted.

In some embodiments, for the compound or salt of Formulas (I), (I-a), (II) or (III), any substituents on B are independently selected at each occurrence from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —S, ═NR¹⁷, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, —S, —NR¹⁷, —N₃, and —CN.

In some embodiments, for the compound or salt of Formulas (I), (I-a), (II) or (III), any substituents on B are independently selected at each occurrence from:
halogen, —OR¹⁷—SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, and —CN;
C₁₋₆ alkyl optionally substituted with one or more substituents independently selected from:
   halogen, —OR¹⁷, —SR¹⁷, —N(R¹⁷)₂, —C(O)R¹⁷, —N(R¹⁷)C(O)R¹⁷, —N(R¹⁷)C(O)OR¹⁷, —N(R¹⁷)S(O)₂(R¹⁷), —S(O)₂N(R¹⁷)₂, ═O, —CN; and
   C₃₋₁₀ carbocycle and 3- to 10-membered heterocycle each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —C(O)N(R¹⁷)₂, —N(R¹⁷)S(O)₂(R¹⁷), —S(O)₂R¹⁷, ═O, ═S, and —CN; and
C₃₋₁₀ carbocycle and 3- to 10-membered heterocycle each of which is optionally substituted with one or more substituents independently selected from:
   halogen, —OR¹⁷, —SR¹⁷, —N(R¹⁷)₂, —C(O)R¹⁷, —C(O)OR¹⁷, —C(O)N(R¹⁷)₂, —(R¹⁷)S(O)₂(R¹⁷), —S(O)R¹⁷, —S(O)₂R¹⁷, ═O, ═S, —CN; and
   C₁₋₆ alkyl optionally substituted with one or more substituents independently selected from: halogen, —OR¹⁷, —SR¹⁷, —N(R¹⁷)₂, ═O, ═S, and —CN.

In some embodiments, for the compound or salt of Formulas (I), (I-a), (II) or (III), any substituents on B are independently selected at each occurrence from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen and 3- to 10-membered heterocycle optionally substituted with one or more substituents independently selected from halogen, C₁₋₆ alkyl, and C₁₋₆ haloalkyl; and
C₃₋₁₀ carbocycle and 3- to 10-membered heterocycle, each of which is optionally substituted with one or more substituents independently selected from: halogen, C₁₋₆ alkyl, and C₁₋₆ haloalkyl.

In some embodiments, for the compound or salt of Formulas (I), (I-a), (II) or (III), any substituents on B are independently selected at each occurrence from:
halogen, —N(R¹⁷)₂, —N(R¹⁷)S(O)₂(R¹⁷), and —CN;
C₁₋₆ alkyl optionally substituted with one or more substituents independently selected from halogen and 3- to 10-membered heterocycle optionally substituted with one or more substituents independently selected from halogen and C₁₋₆ alkyl; and
3- to 10-membered heterocycle optionally substituted with one or more substituents independently selected from C₁₋₆ alkyl and C₁₋₆ haloalkyl.

In some embodiments, for the compound or salt of Formulas (I), (I-a), (II) or (III), B is substituted with 1, 2, 3, 4, or 5 substituents. In some embodiments, B is substituted with 1, 2, 3, or 4 substituents. In some embodiments, B is substituted with 1, 2, or 3 substituents. In some embodiments, B is substituted with 2 or 3 substituents. In some embodiments, B is substituted with 1 or 2 substituents. In some embodiments, B is substituted with 1 substituent. In some embodiments, B is substituted with 2 substituents. In some embodiments, B is substituted with 3 substituents. In some embodiments, B is substituted with 4 substituents. In some embodiments, B is substituted with 5 substituents.

In some embodiments, for the compound or salt of Formulas (I), (I-a), (II) or (III), B is selected from: phenyl, naphthyl, C₃₋₁₀ cycloalkyl, 5- to 6-membered monocyclic heterocycle, and 7- to 10-membered bicyclic heterocycle, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, —O, and CN.

In some embodiments, for the compound or salt of Formulas (I), (I-a), (II) or (III), B is selected from: phenyl, naphthyl, C₃₋₁₀ cycloalkyl, 5- to 6-membered monocyclic heterocycle, and 7- to 10-membered bicyclic heterocycle, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, —O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from: phenyl, naphthyl, C₃₋₁₀ cycloalkyl, 5- to 6-membered monocyclic heterocycle, and 7- to 10-membered bicyclic heterocycle, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), B is selected from: phenyl, naphthyl, C₃₋₁₀ cycloalkyl, 5- to 6-membered monocyclic heterocycle, and 7- to 10-membered bicyclic heterocycle, any of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

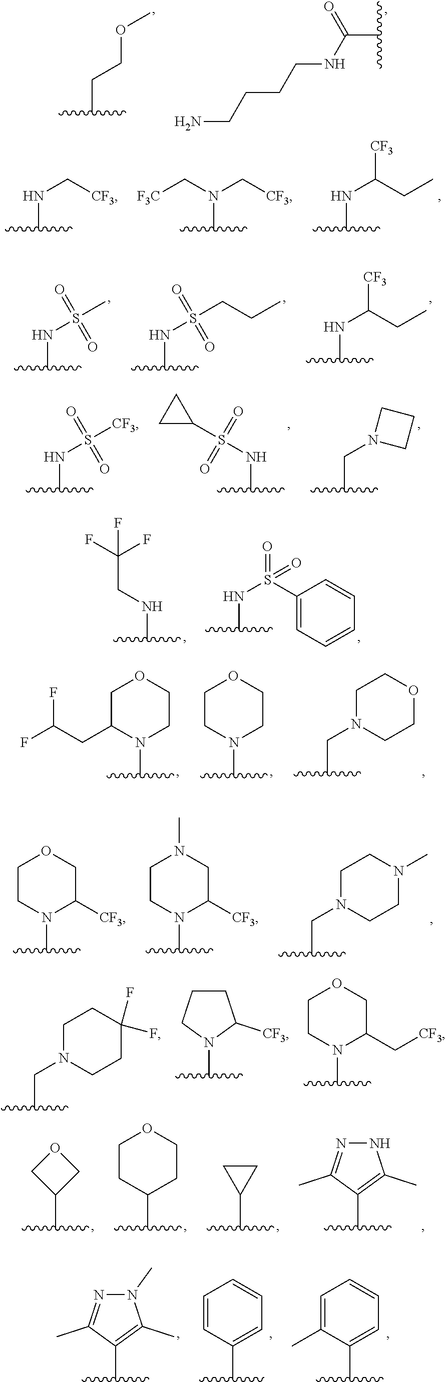

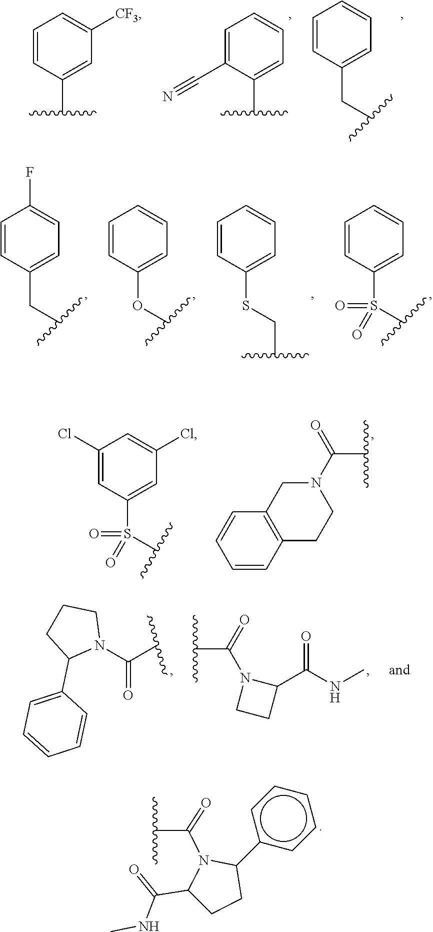

In some embodiments, for the compound or salt of Formulas (I), (I-a), (II) or (III), B is selected from: phenyl, naphthyl, cyclopropyl, cyclohexyl, bicyclo[2.2.2]octane, adamantane, thiazole, pyrazole, pyridine, indole, indazole, benzimidazole, indoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, —O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, ═O, and CN.

In some embodiments, for the compound or salt of Formulas (I), (I-a), (II) or (III), B is selected from: phenyl, naphthyl, cyclopropyl, cyclohexyl, bicyclo[2.2.2]octane, adamantane, thiazole, pyrazole, pyridine, indole, indazole, benzimidazole, indoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, ═O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from: phenyl, naphthyl, cyclopropyl, cyclohexyl, bicyclo[2.2.2]octane, adamantane, thiazole, pyrazole, pyridine, indole, indazole, benzimidazole, indoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), B is selected from: phenyl, naphthyl, cyclopropyl, cyclohexyl, bicyclo[2.2.2]octane, adamantane, thiazole, pyrazole, pyridine, indole, indazole, benzimidazole, indoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted any of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

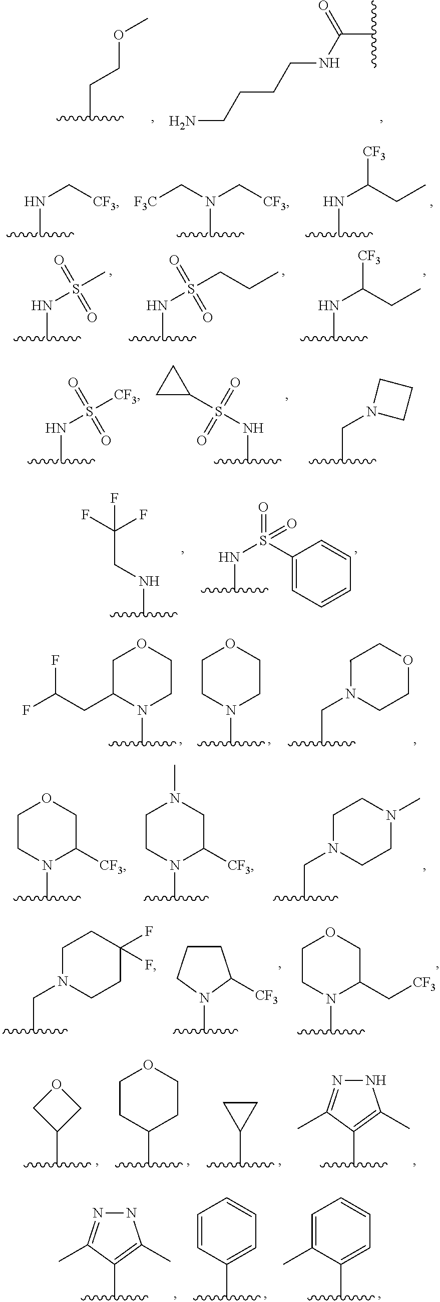

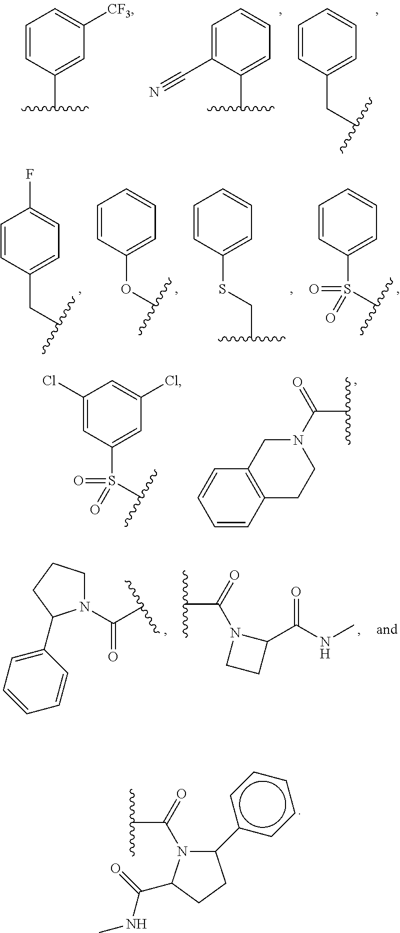

In some embodiments, for the compound or salt of Formulas (I), (I-a), (II) or (III), B is selected from: C₃₋₇ monocyclic carbocycle and 5- to 6-membered monocyclic heterocycle, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, and —CN;
C₁₋₆ alkyl, C₂₋₆ alkenyl, and C₂₄ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, —O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, ═O, and —CN.

In some embodiments, for the compound or salt of Formulas (I), (I-a), (II) or (III), B is selected from: C₃₋₇ monocyclic carbocycle and 5- to 6-membered monocyclic heterocycle, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, —O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from: C₃₋₇ monocyclic carbocycle and 5- to 6-membered monocyclic heterocycle, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), B is selected from: C₃₋₇ monocyclic carbocycle and 5- to 6-membered monocyclic heterocycle, any of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

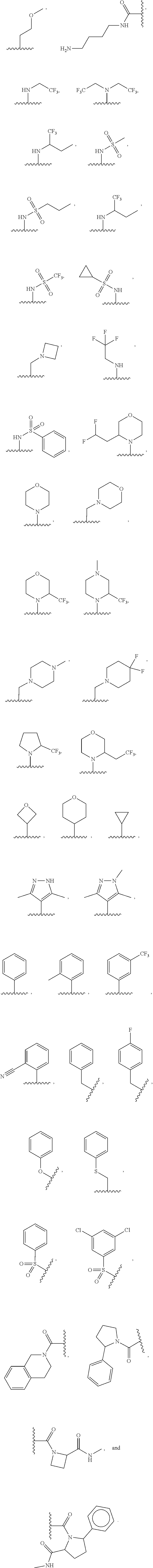

In some embodiments, for the compound or salt of Formulas (I), (I-a), (II) or (III), B is selected from: phenyl, cyclopropyl, cyclohexyl, thiazole, pyrazole, pyridine, indole, indazole, tetrahydropyran, azetidine, pyrrolidine, piperidine, and azepane, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, —O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, ═O, and CN.

In some embodiments, for the compound or salt of Formulas (I), (I-a), (II) or (III), B is selected from: phenyl, cyclopropyl, cyclohexyl, thiazole, pyrazole, pyridine, indole, indazole, tetrahydropyran, azetidine, pyrrolidine, piperidine, and azepane, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, ═O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from: phenyl, cyclopropyl, cyclohexyl, thiazole, pyrazole, pyridine, indole, indazole, tetrahydropyran, azetidine, pyrrolidine, piperidine, and azepane, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), B is selected from: phenyl, cyclopropyl, cyclohexyl, thiazole, pyrazole, pyridine, indole, indazole, tetrahydropyran, azetidine, pyrrolidine, piperidine, and azepane, any of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

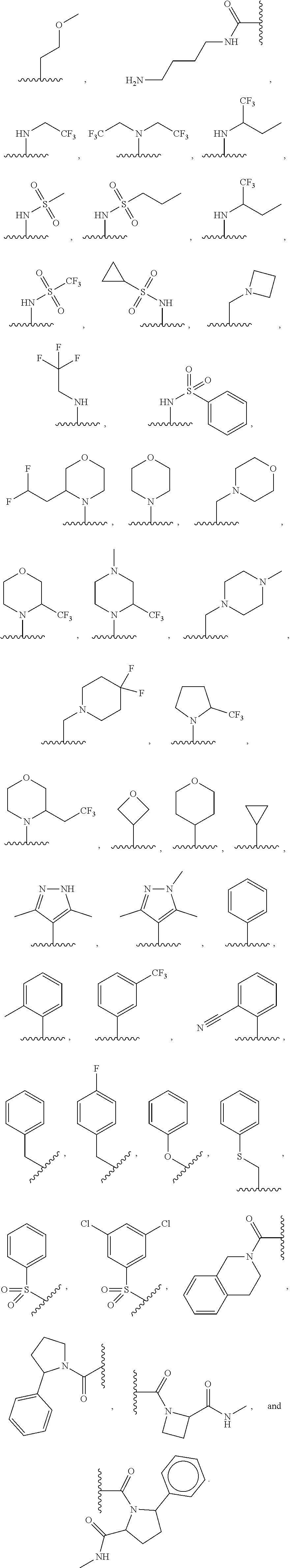

In some embodiments, for the compound or salt of Formulas (I), (I-a), (II) or (III), B is selected from: C₇-10 bicyclic carbocycle and 7- to 10-membered bicyclic heterocycle, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, —S, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, ═O, and —CN.

In some embodiments, for the compound or salt of Formulas (I), (I-a), (II) or (III), B is selected from: C₇-10 bicyclic carbocycle, and 7- to 10-membered bicyclic heterocycle, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, ═O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from: C₇₋₁₀ bicyclic carbocycle and 7- to 10-membered bicyclic heterocycle, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂R¹⁷, —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), B is selected from: C₇₋₁₀ bicyclic carbocycle and 7- to 10-membered bicyclic heterocycle, any of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

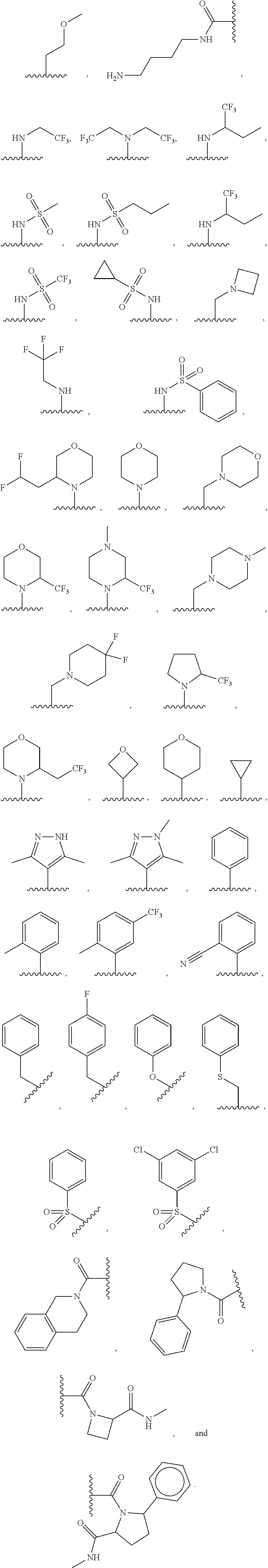

In some embodiments, for the compound or salt of Formulas (I), (I-a), (II) or (III), B is selected from: naphthyl, bicyclo[2.2.2]octane, adamantane, indole, indazole, benzimidazole, indoline, 1,3-dihydrobenzo[c]isothiazole, quinuclidine, and 8-azabicyclo[3.2.1]octane, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, ═S, ═NR¹⁷, and —CN; and
C₃₋₁₀ carbocycle and 3- to 10-membered heterocycle, each of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, ═S, and —CN;
   C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂N(R¹⁷)₂, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —N(R¹⁷)₂, ═O, and —CN.

In some embodiments, for the compound or salt of Formulas (I), (I-a), (II) or (III), B is selected from: naphthyl, bicyclo[2.2.2]octane, adamantane, indole, indazole, benzimidazole, indoline, 1,3-dihydrobenzo[c]isothiazole, quinuclidine, and 8-azabicyclo[3.2.1]octane, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁷, —SR¹⁷, —N(R¹⁷)₂, —C(O)R¹⁷, —C(O)N(R¹⁷)₂, —N(R¹⁷)C(O)R¹⁷, ═O, and —CN;
   C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —N(R¹⁷)₂, —C(O)R¹⁷; ═O, and —CN; and
C₃₋₁₀ carbocycle and 3- to 10-membered heterocycle, each of which is optionally substituted with one or more substituents independently selected from:
   halogen, —OR¹⁷, —N(R¹⁷)₂, —C(O)R¹⁷, —C(O)N(R¹⁷)₂, —N(R¹⁷)C(O)R¹⁷, —O, and —CN;
   C₁₋₆ alkyl optionally substituted with one or more substituents independently selected from: halogen, —OR¹⁷, —N(R¹⁷)₂, —C(O)R¹⁷, —C(O)N(R¹⁷)₂, —N(R¹⁷)C(O)R¹⁷, ═O, and —CN; and
   C₃₋₁₀ carbocycle and 3- to 10-membered heterocycle each of which is optionally substituted with one or more substituents independently selected from halogen, C₁₋₆ alkyl, C₁₋₆ haloalkyl, and —CN.

In some embodiments, for the compound or salt of Formulas (I), (I-a), (II) or (III), B is selected from: naphthyl, bicyclo[2.2.2]octane, adamantane, indole, indazole, benzimidazole, indoline, 1,3-dihydrobenzo[c]isothiazole, quinuclidine, and 8-azabicyclo[3.2.1]octane, any of which is optionally substituted with one or more substituents independently selected from:
halogen, —OR¹⁷, —N(R¹⁷)₂, —C(O)R¹⁷, —C(O)N(R¹⁷)₂, —N(R¹⁷)S(O)₂R¹⁷, —S(O)₂R¹⁷, and —CN;
C₁₋₆ alkyl optionally substituted with one or more substituents independently selected from: halogen, —SR¹⁷, C₃₋₁₀ carbocycle, and 3- to 10-membered heterocycle, wherein the C₃₋₁₀ carbocycle and 3- to 10-membered heterocycle is optionally substituted with one or more substituents independently selected from: halogen, C₁₋₆ alkyl, and C₁₋₆ haloalkyl; and
C₃₋₁₀ carbocycle and 3- to 10-membered heterocycle, each of which is optionally substituted with one or more substituents independently selected from: ═O; —CN; C₁₋₆ alkyl optionally substituted with one or more substituents independently selected from halogen, and —OR¹⁷; and C₃₋₁₀ carbocycle.

In some embodiments, for the compound or salt of Formulas (I), (I-a), (II) or (III), B is selected from: naphthyl, bicyclo[2.2.2]octane, adamantane, indole, indazole, benzimidazole, indoline, 1,3-dihydrobenzo[c]isothiazole, quinuclidine, and 8-azabicyclo[3.2.1]octane, any of which is optionally substituted with one or more substituents independently selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

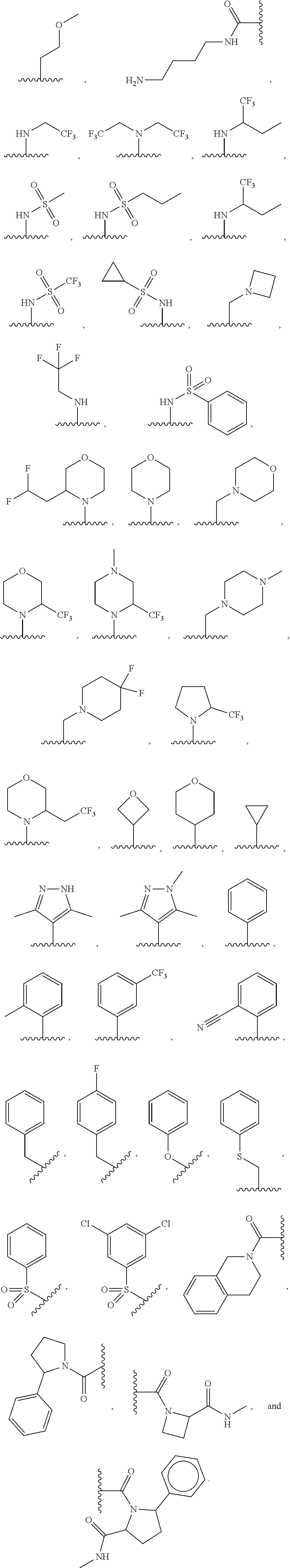

In some embodiments, for the compound or salt of Formulas (I), (I-a), (II) or (III), B is selected from:

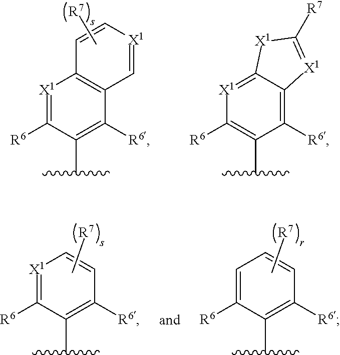

wherein each X¹ is independently Nor C(R⁸);
R⁶, R^{6′}, R⁷, and R⁸ are each independently selected from:
   hydrogen; halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, —N₃, —CN, and
   C₁₋₆ alkyl optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, —CN; and
      C₃₋₁₀ carbocycle and 3- to 10-membered heterocycle each of which is optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, and —CN; and
   C₃₋₁₀ carbocycle and 3- to 10-membered heterocycle each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, and —CN;
s is 0, 1, 2, or 3; and
r is 0, 1, or 2.

In some embodiments, for the compound or salt of Formulas (I), (I-a), (II) or (III), B is selected from:

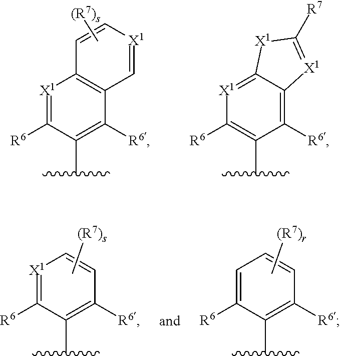

wherein each X¹ is independently N or C(R⁸);
R⁶ and R^{6′} are each independently selected from:
   hydrogen, halogen, —OR¹⁷, —SR¹⁷, —N(R⁴¹)₂, —C(O)R⁴¹, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, —N₃, —CN, and C₁₋₆ alkyl optionally substituted with one or more substituents independently selected from: halogen, —OH, —O—C₁₋₆ alkyl, —O—C₁₋₆ haloalkyl —NH₂, —NO₂, ═O, CN; and
R⁷ and R⁸ are each independently selected from:
   hydrogen; halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, —N₃, —CN, and
   C₁₋₆ alkyl optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, —CN; and
      C₃₋₁₀ carbocycle and 3- to 10-membered heterocycle each of which is optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, and —CN; and
   C₃₋₁₀ carbocycle and 3- to 10-membered heterocycle each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, and —CN;
s is 0, 1, 2, or 3; and
r is 0, 1, or 2.

In some embodiments, for the compound or salt of Formulas (I), (I-a), (II) or (III), B is selected from:

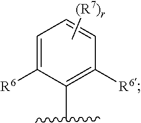

wherein each X¹ is independently Nor C(R⁸);
R⁶, R^{6′}, R⁷, and R⁸ are each independently selected from:
   hydrogen; halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, —N₃, —CN, and
   C₁₋₆ alkyl optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, —CN; and
      C₃₋₁₀ carbocycle and 3- to 10-membered heterocycle each of which is optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, —N₃, and —CN; and
   C₃₋₁₀ carbocycle and 3- to 10-membered heterocycle each of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —S, —N₃, and —CN; and
r is 0, 1, or 2.

In some embodiments, for the compound or salt of Formulas (I), (I-a), (II) or (III), s is 0, 1, 2, or 3. In some embodiments, s is 0, 1, or 2. In some embodiments, s is 1, 2 or 3. In some embodiments, s is 0 or 1. In some embodiments, s is 1 or 2. In some embodiments, s is 2 or 3. In some embodiments, s is 0. In some embodiments, s is 1. In some embodiments, s is 2. In some embodiments, s is 3.

In some embodiments, for the compound or salt of Formulas (I), (I-a), (II) or (III), r is 0, 1, or 2. In some embodiments, r is 0 or 1. In some embodiments, r is 1 or 2. In some embodiments, r is 0. In some embodiments, r is 1. In some embodiments, r is 2.

In some embodiments, for the compound or salt of Formulas (I), (I-a), (II) or (III), R⁶ and R^{6′} are each independently selected from: hydrogen; halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —CN, and C₁₋₆ alkyl optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
C₃₋₁₀ carbocycle and 3- to 10-membered heterocycle each of which is optionally substituted with one or more substituents independently selected from: halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN.

In some embodiments, for the compound or salt of Formulas (I), (I-a), (II) or (III), R⁶ and R^{6′} are each independently selected from: hydrogen, halogen, —OR¹⁷, —N(R¹⁷)₂, and C₁₋₆ alkyl optionally substituted with one or more substituents independently selected from: halogen, —OR¹⁷, —N(R¹⁷)₂, C₃₋₁₀ carbocycle, and 3- to 10-membered heterocycle each of which is optionally substituted with one or more substituents independently selected from: halogen, —OR¹⁷, —N(R¹⁷)₂, ═O, and —CN. In some embodiments, R⁶ and R^{6′} are each independently selected from: hydrogen, halogen, and C₁₋₆ alkyl optionally substituted with one or more substituents independently selected from: halogen, C₃₋₁₀ carbocycle, and 3- to 10-membered heterocycle each of which is optionally substituted with one or more substituents independently selected from: halogen, —O, and —CN. In some embodiments, R⁶ and R^{6′} are each independently selected from: halogen, and C₁₋₆ alkyl optionally substituted with one or more substituents independently selected from: halogen and 3- to 10-membered heterocycle each of which is optionally substituted with one or more substituents independently selected from: halogen.

In some embodiments, for the compound or salt of Formulas (I), (I-a), (II) or (III), R⁶, and R^{6′} are each independently selected from: hydrogen, halogen, —OR¹⁷, —SR¹⁷, —N(R⁴¹)₂, —C(O)R⁴¹, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, —N₃, —CN, and C₁₋₆ alkyl optionally substituted with one or more substituents independently selected from: halogen, —OH, —O—C₁₋₆ alkyl, —O—C₁₋₆ haloalkyl —NH₂, —NO₂, ═O, and —CN. In some embodiments, R⁶ and R^{6′} are each independently selected from: hydrogen, halogen, —OR¹⁷, —SR¹⁷, —N(R⁴¹)₂, —C(O)R⁴¹, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)R¹⁷, —S(O)₂R¹⁷, —S(O)₂N(R¹⁷)₂, —CN, and C₁₋₆ alkyl optionally substituted with one or more substituents independently selected from: halogen, —OH, —O—C₁₋₆ alkyl, —O—C₁₋₆ haloalkyl, ═O, and —CN. In some embodiments, R⁶ and R^{6′} are each independently selected from: hydrogen, halogen, —OR¹⁷, —SR¹⁷, —N(R⁴¹)₂, —C(O)R⁴¹, —C(O)OR¹⁷, —OC(O)R¹⁷, —C(O)N(R¹⁷)₂, —N(R¹⁷)C(O)R¹⁷, —CN, and C₁₋₆ alkyl optionally substituted with one or more substituents independently selected from: halogen, —OH, —O—C₁₋₆ alkyl, and ═O. In some embodiments, R⁶ and R^{6′} are each independently selected from: hydrogen, halogen, —OR¹⁷, —SR¹⁷, —N(R⁴¹)₂, —CN, and C₁₋₆ alkyl optionally substituted with one or more substituents independently selected from: halogen and ═O. In some embodiments, R⁶ and R^{6′} are each independently selected from: hydrogen, halogen, —OR¹⁷, —N(R⁴¹)₂, —CN, C₁₋₆ alkyl, and C₁₋₆ haloalkyl. In some embodiments, R⁶ and R^{6′} are each independently selected from: hydrogen, halogen, C₁₋₆ alkyl, and C₁₋₆ haloalkyl. In some embodiments, R⁶ and R^{6′} are each independently selected from: halogen, C₁₋₆ alkyl, and C₁₋₆ haloalkyl. In some embodiments, R⁶ and R^{6′} are each independently selected from halogen. In some embodiments, R⁶ and R^{6′} are each fluoro.

In some embodiments, for the compound or salt of Formulas (I), (I-a), (II) or (III), R⁷, and R⁸ are each independently selected from:
hydrogen; halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, and —CN;
C₁₋₆ alkyl optionally substituted with one or more substituents independently selected from:
   halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle each of which is optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN; and
C₃₋₁₀ carbocycle and 3- to 10-membered heterocycle each of which is optionally substituted with one or more substituents independently selected from:
   halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)R¹⁷, —S(O)₂R¹⁷, —S(O)₂N(R¹⁷)₂, ═O, and —CN.

In some embodiments, for the compound or salt of Formulas (I), (I-a), (II) or (III), R⁷, and R⁸ are each independently selected from:
hydrogen; halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, and —CN;
C₁₋₆ alkyl optionally substituted with one or more substituents independently selected from:
   halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN;
   C₃₋₁₀ carbocycle and 3- to 10-membered heterocycle each of which is optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, ═O, and —CN; and
C₃₋₁₀ carbocycle and 3- to 10-membered heterocycle each of which is optionally substituted with one or more substituents independently selected from:
   halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)R¹⁷, —S(O)₂R¹⁷, —S(O)₂N(R¹⁷)₂, ═O, and —CN.

In some embodiments, for the compound or salt of Formulas (I), (I-a), (II) or (III), R⁷, and R⁸ are each independently selected from:
halogen, —OR¹⁷, —N(R¹⁷)₂, —C(O)R¹⁷, —C(O)N(R¹⁷)₂, —N(R¹⁷)S(O)₂(R¹⁷), —S(O)₂R¹⁷, and —CN;
C₁₋₆ alkyl optionally substituted with one or more substituents independently selected from: halogen and 3- to 10-membered heterocycle optionally substituted with one or more substituents independently selected from halogen, C₁₋₆ alkyl, and C₁₋₆ haloalkyl; and
C₃₋₁₀ carbocycle and 3- to 10-membered heterocycle, each of which is optionally substituted with one or more substituents independently selected from: halogen, C₁₋₆ alkyl, and C₁₋₆ haloalkyl.

In some embodiments, for the compound or salt of Formulas (I), (I-a), (II) or (III), R⁷, and R⁸ are each independently selected from:
halogen, —N(R¹⁷)₂, —N(R¹⁷)S(O)₂(R¹⁷), and —CN;
C₁₋₆ alkyl optionally substituted with one or more substituents independently selected from halogen and 3- to 10-membered heterocycle optionally substituted with one or more substituents independently selected from halogen and C₁₋₆ alkyl; and
3- to 10-membered heterocycle optionally substituted with one or more substituents independently selected from C₁₋₄ alkyl and C₁₋₆ haloalkyl

In some embodiments, for the compound or salt of Formulas (I), (I-a), (II) or (III), B is selected from:

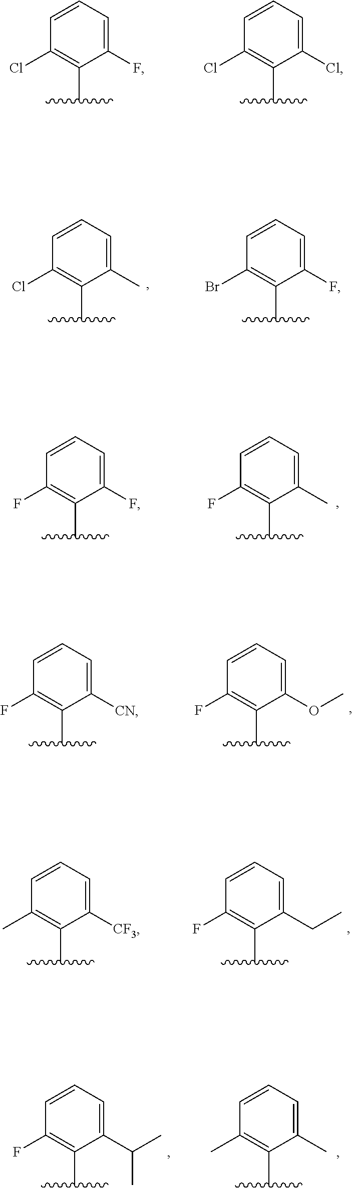

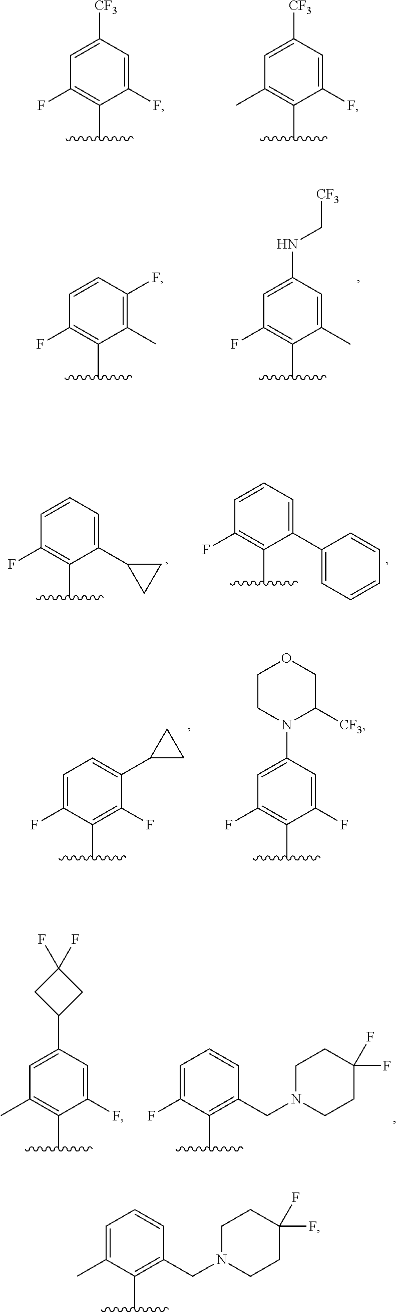

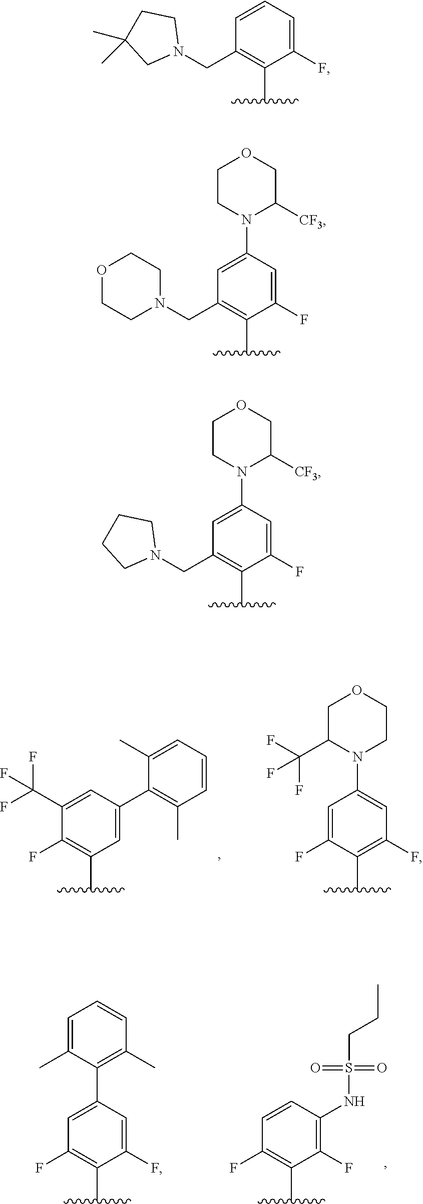

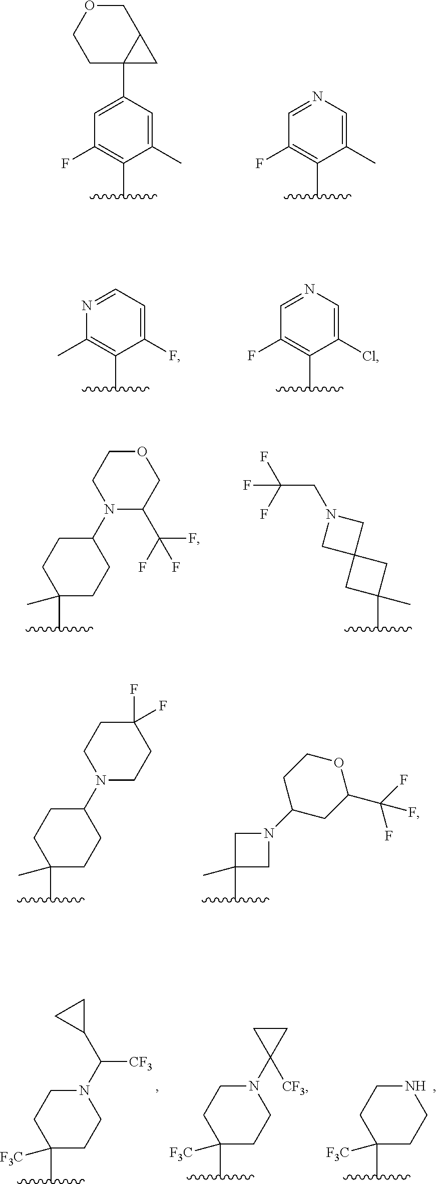

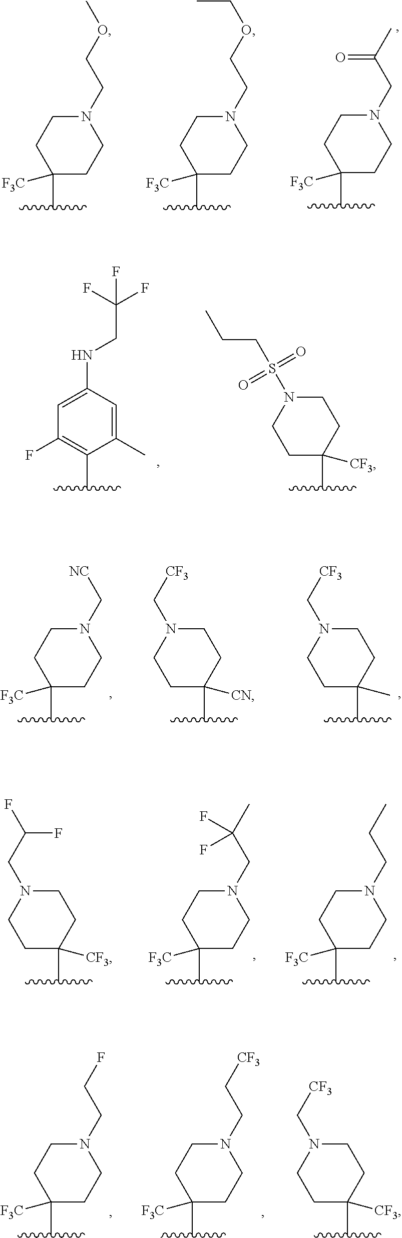

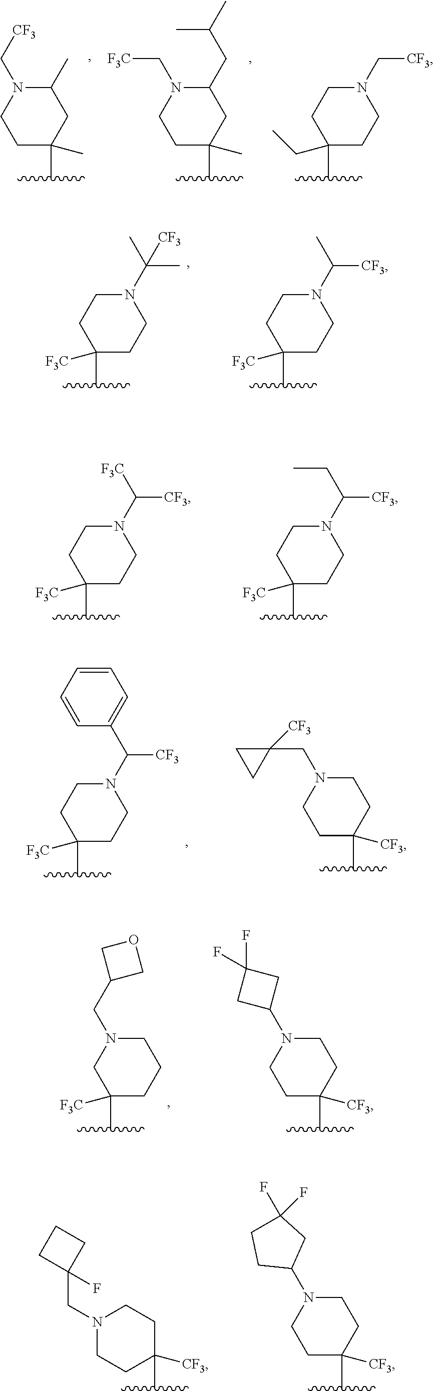

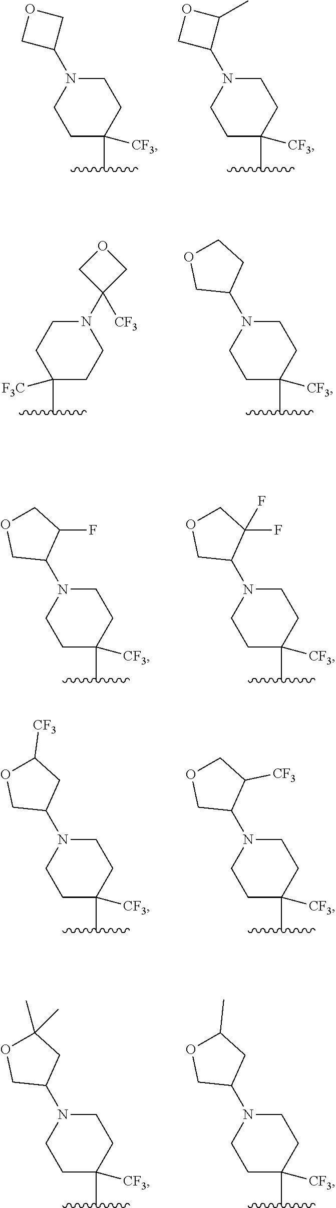

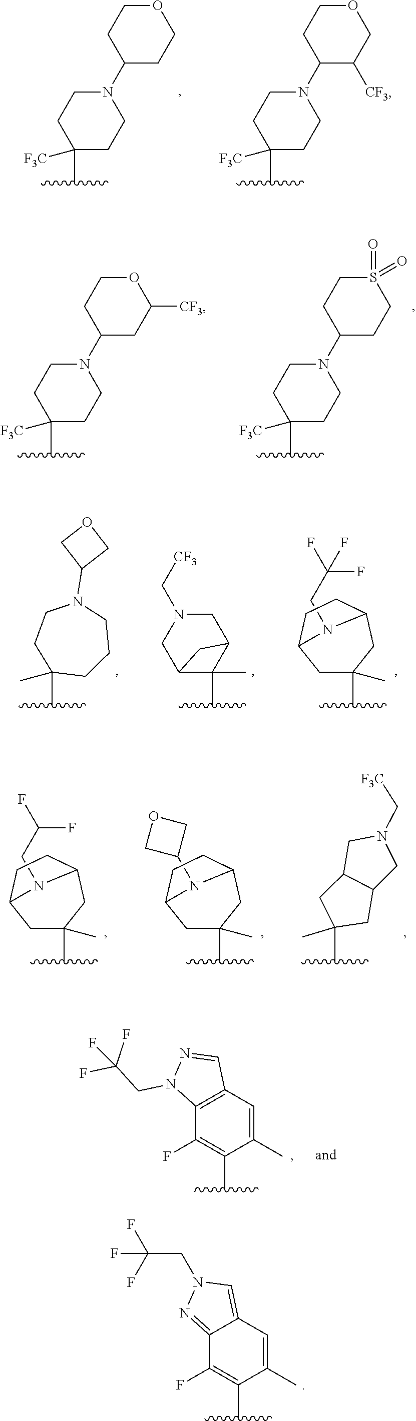

In some embodiments, for the compound or salt of Formulas (I), (I-a), (II) or (III), B is selected from

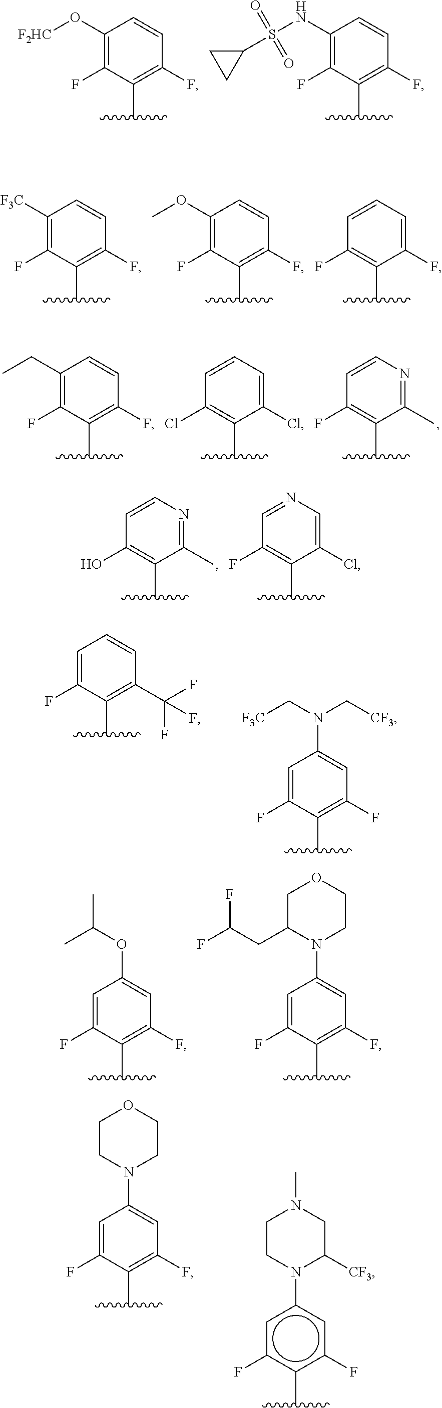

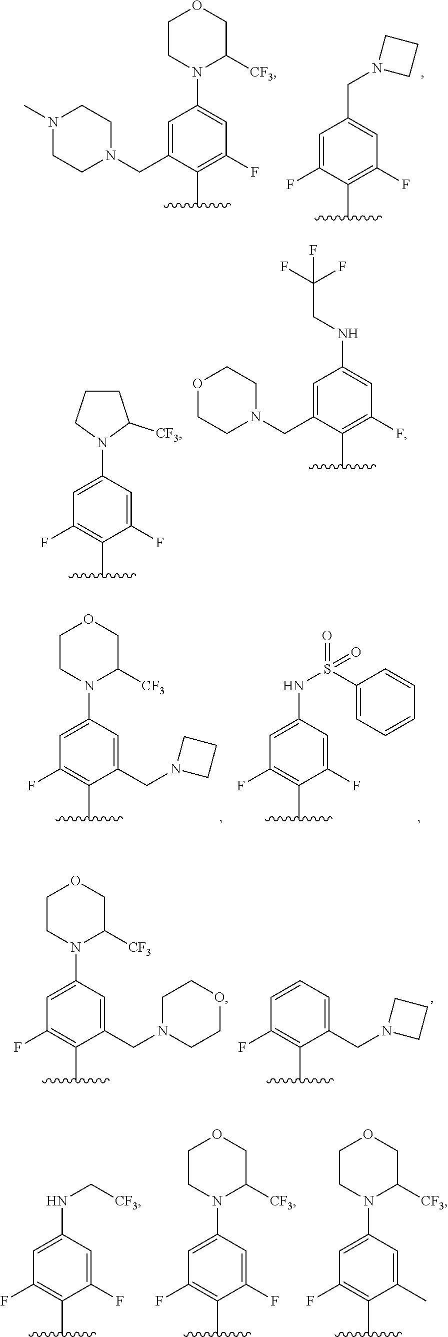

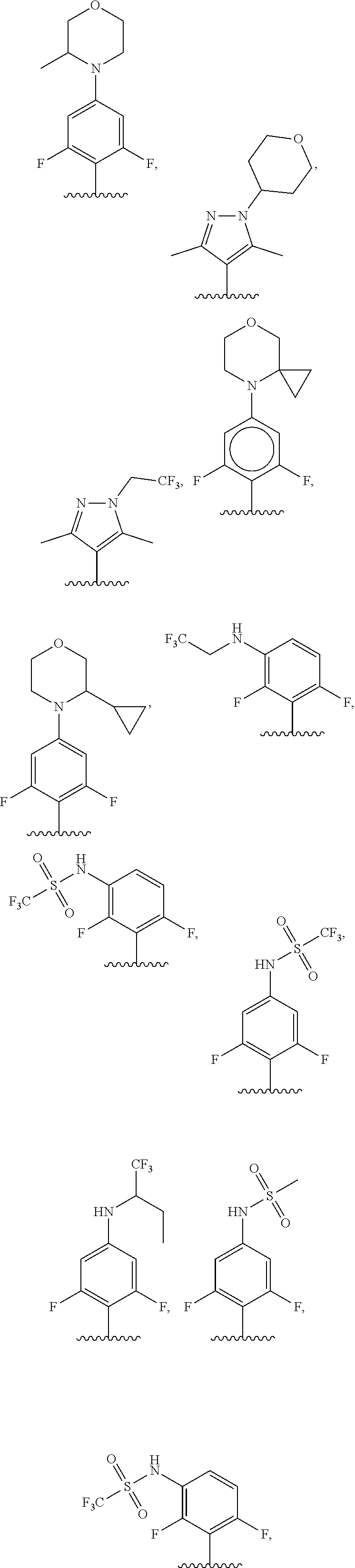

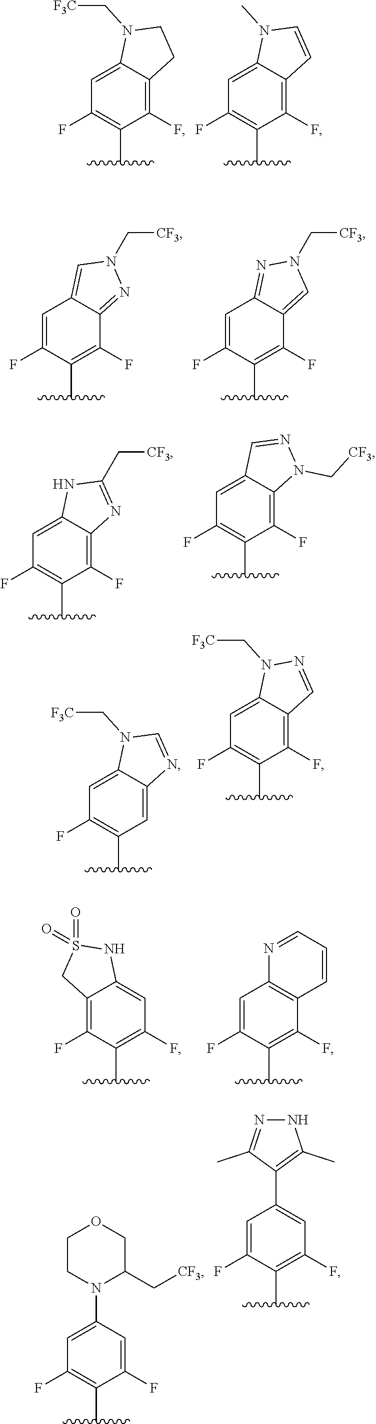

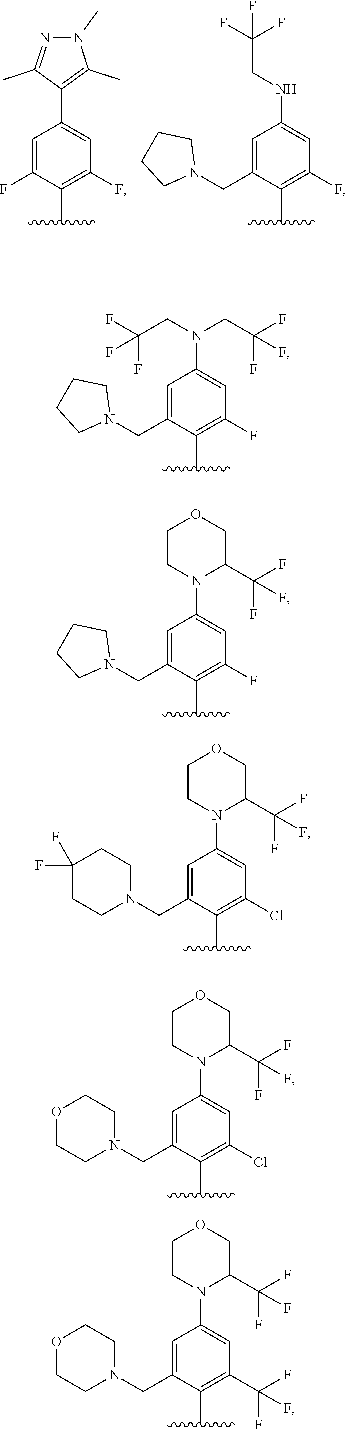

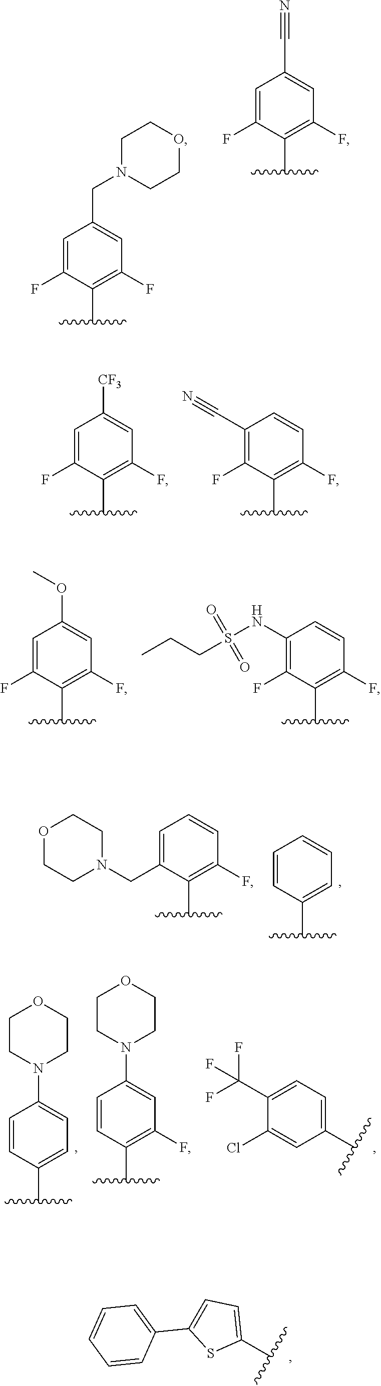

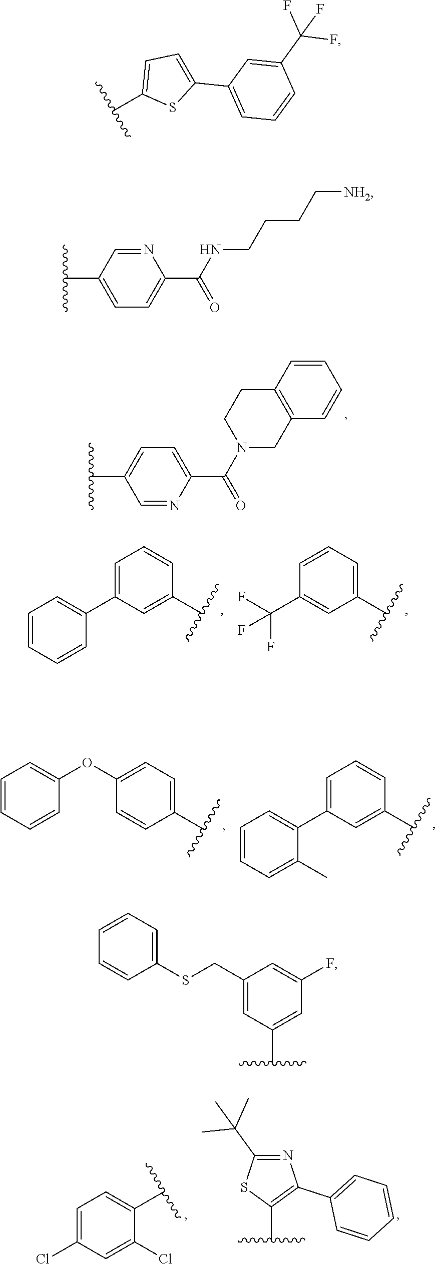

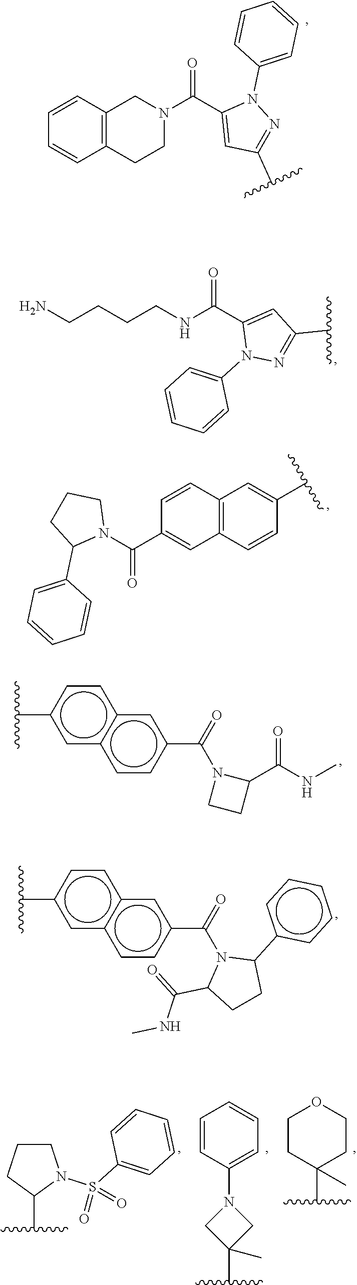

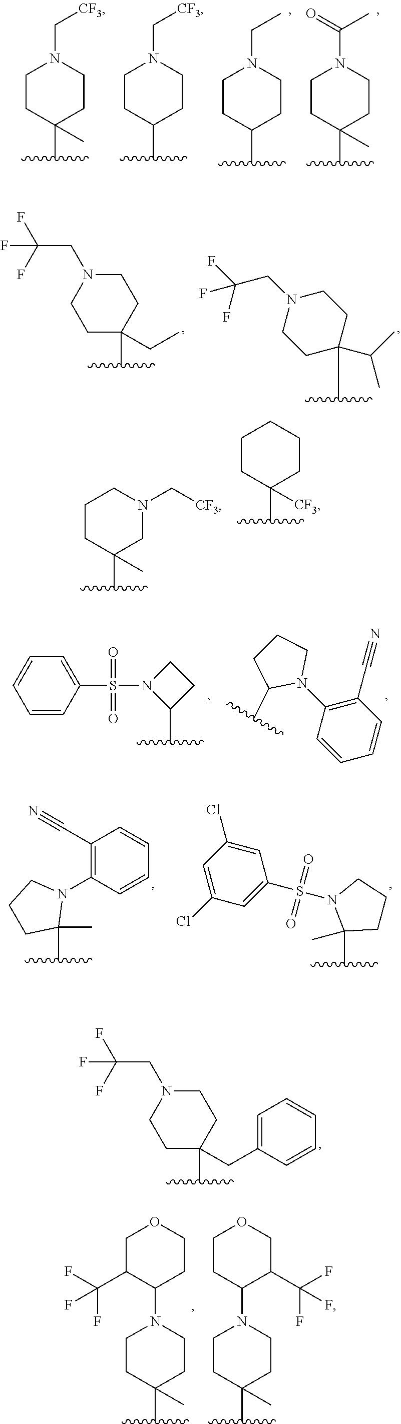

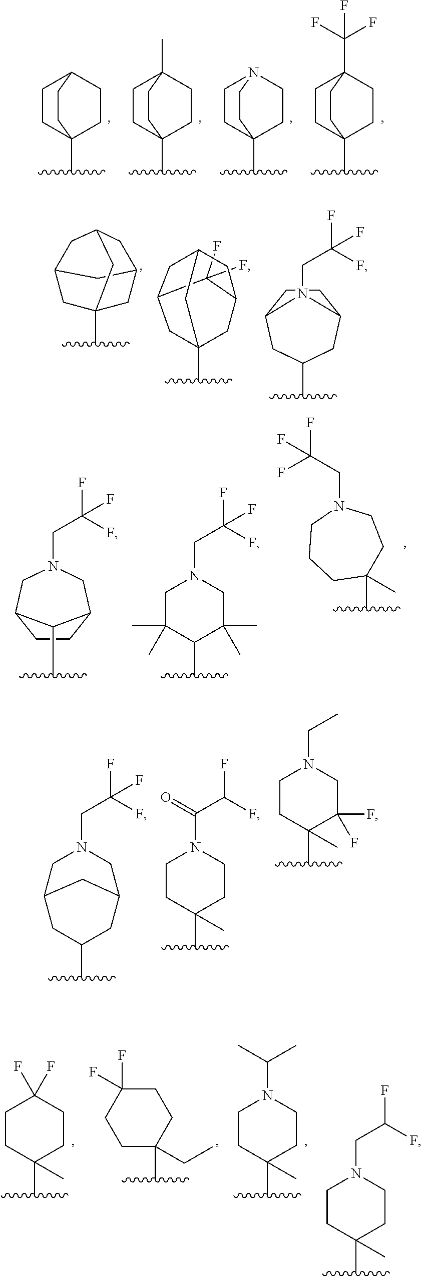

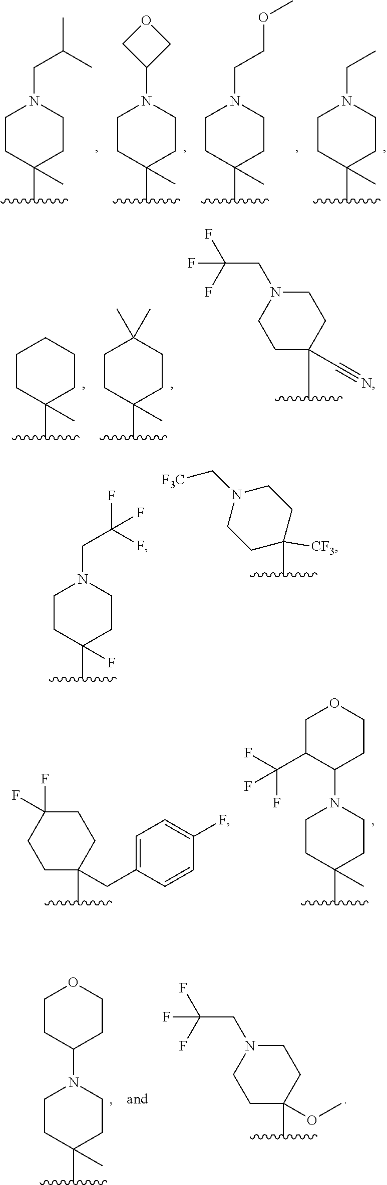

In some embodiments, for the compound or salt of Formulas (I), (I-a), (II) or (III), B is selected from

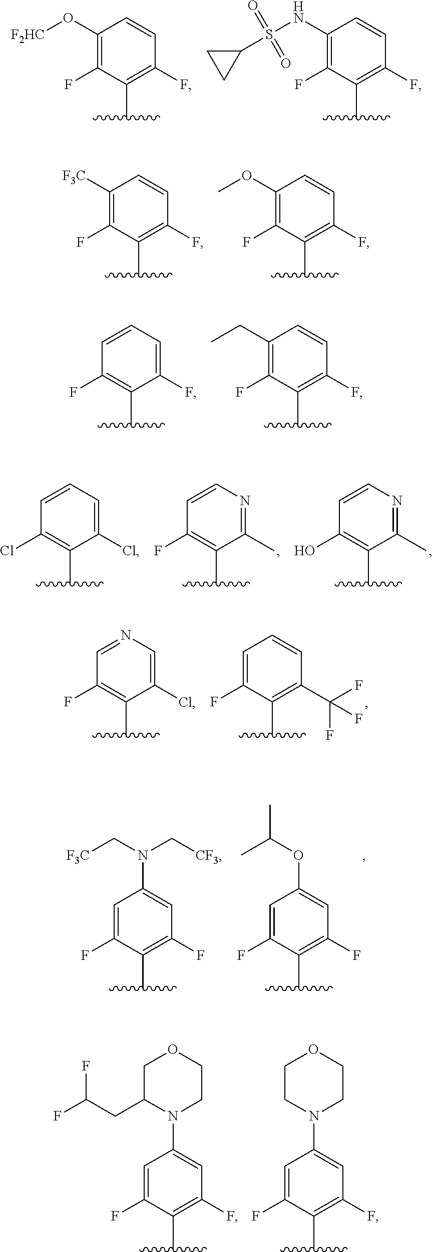

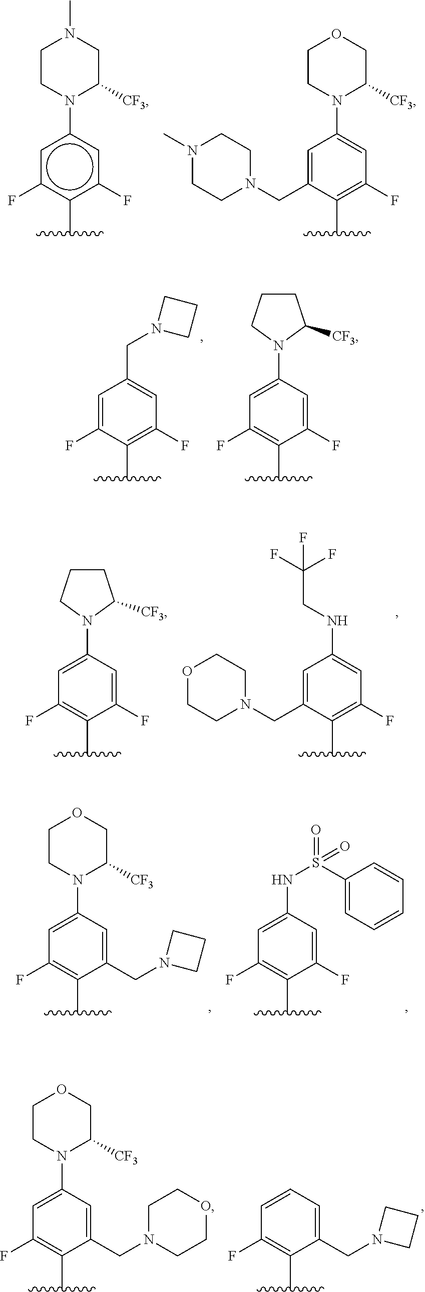

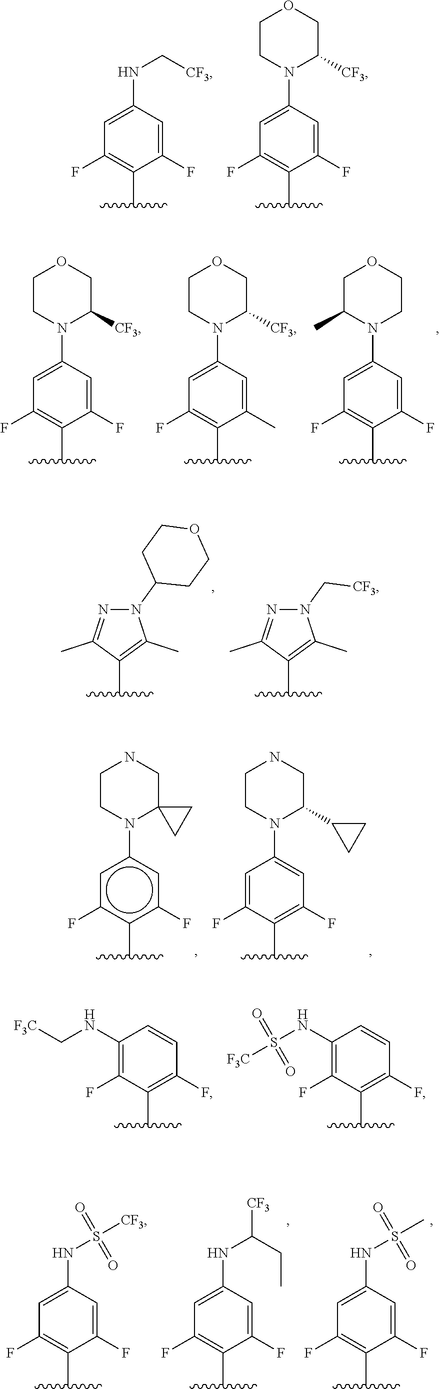

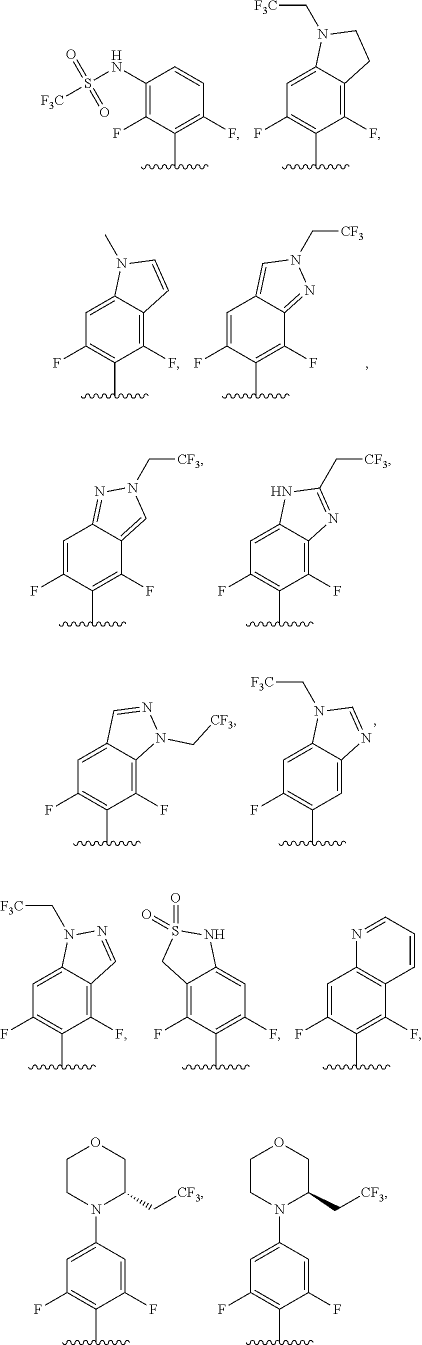

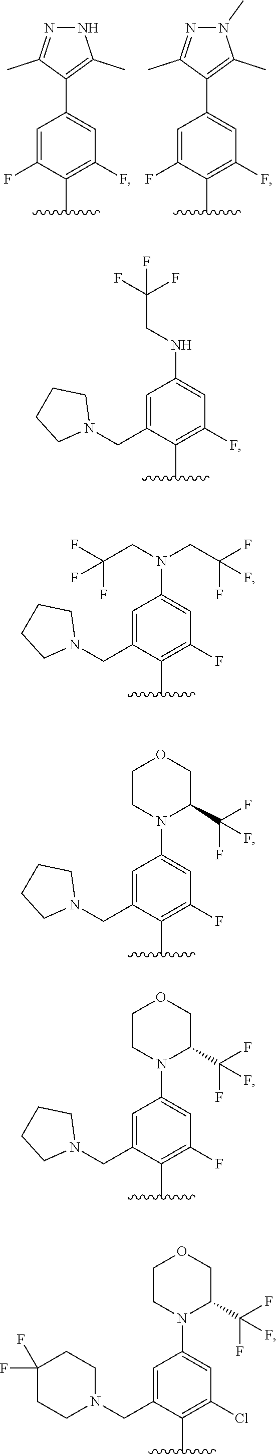

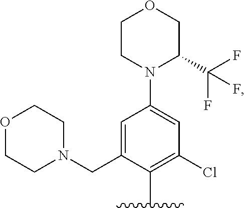 [SMILES: *c1c(Cl)cc(N2CCOC[C@@H]2C(F)(F)F)cc1CN1CCOCC1]

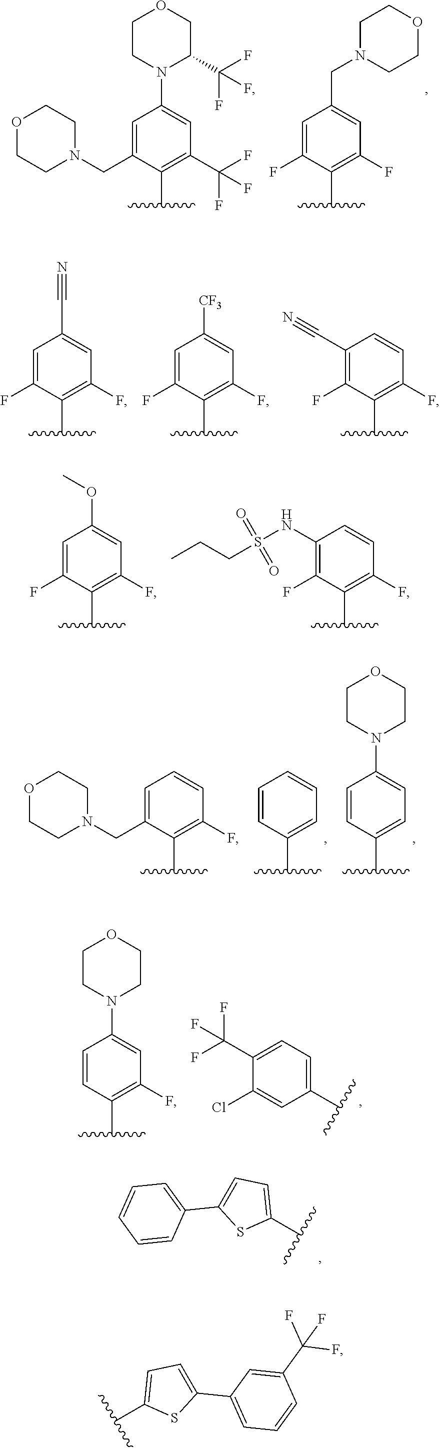

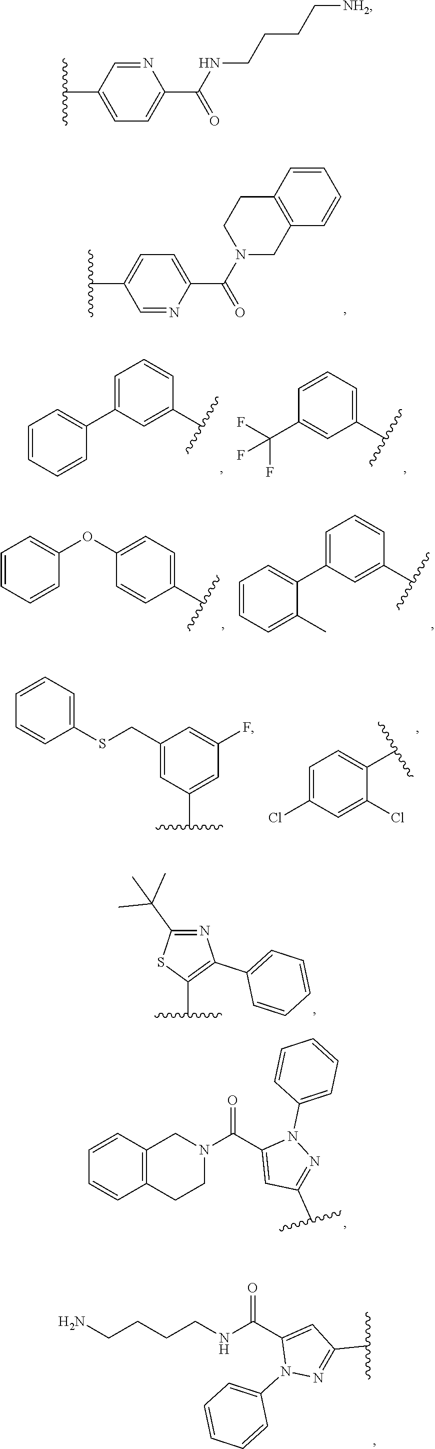

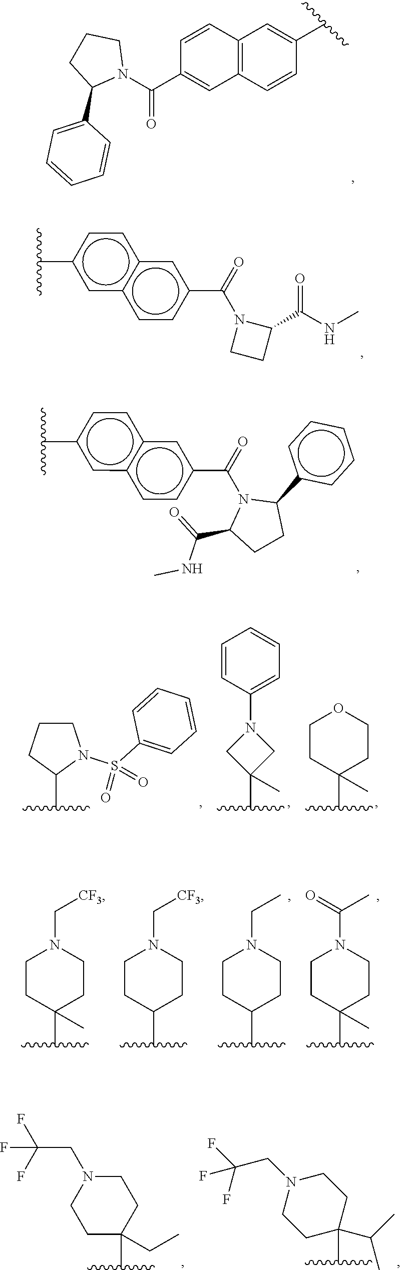

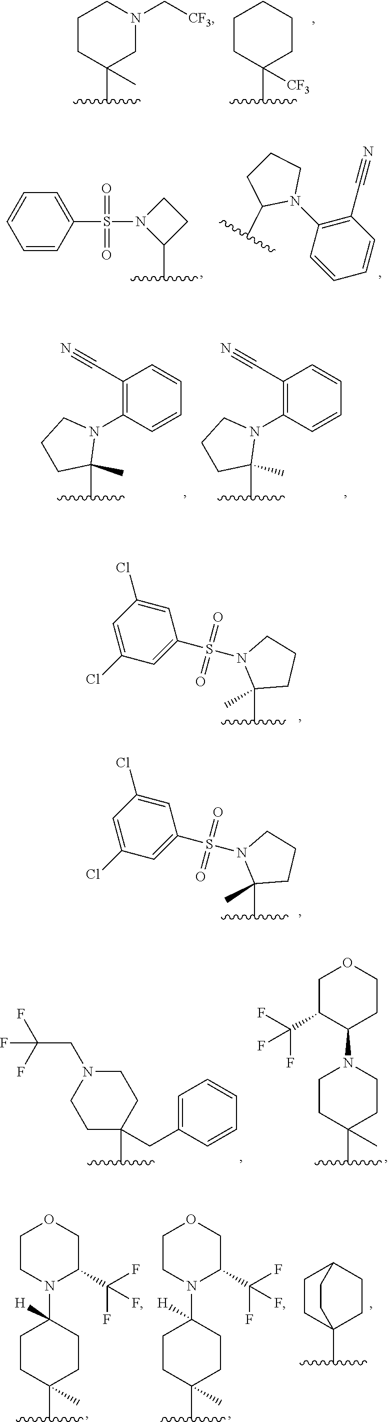

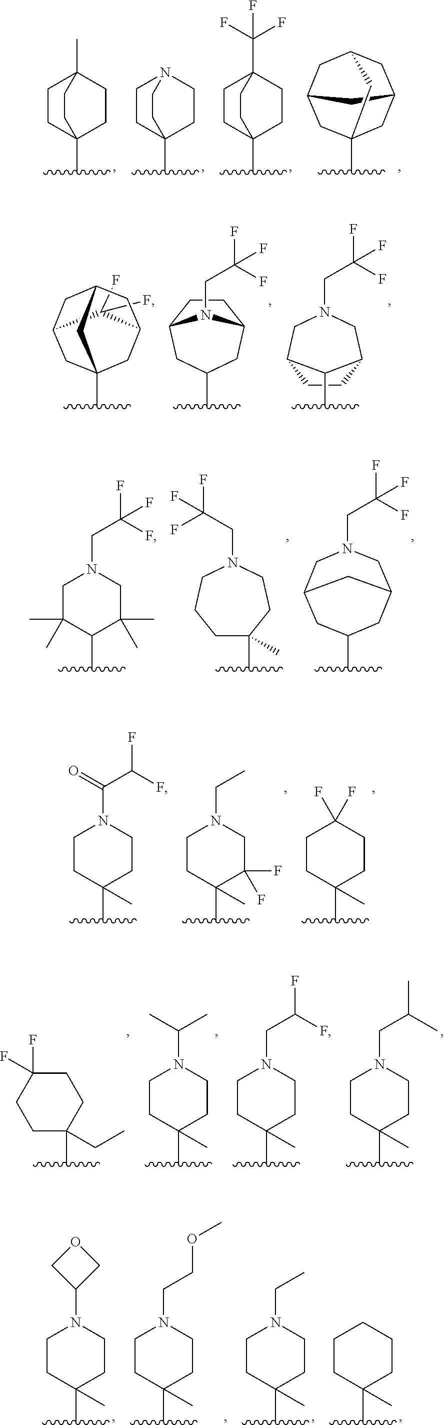

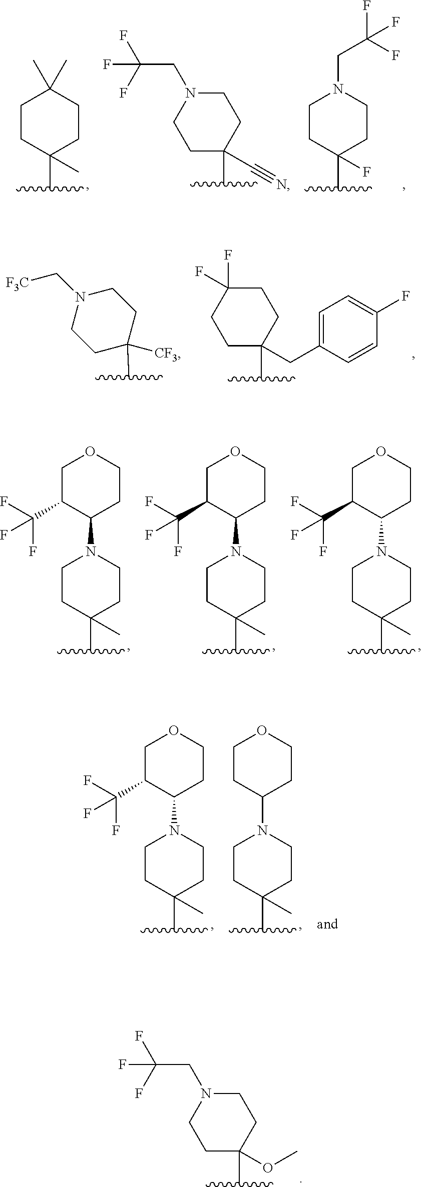

In some embodiments, for the compound or salt of Formulas (I), (I-a), (II) or (III), B is selected from:

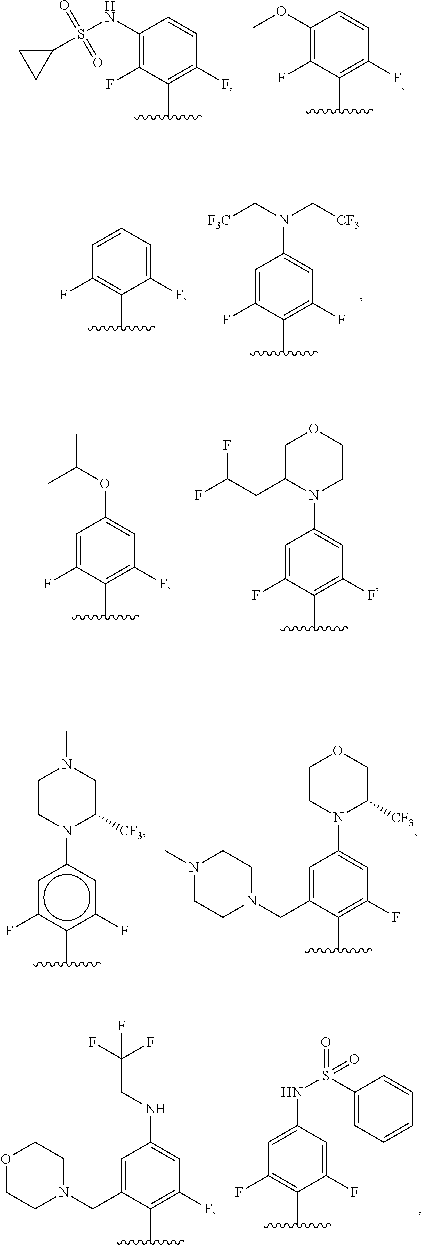

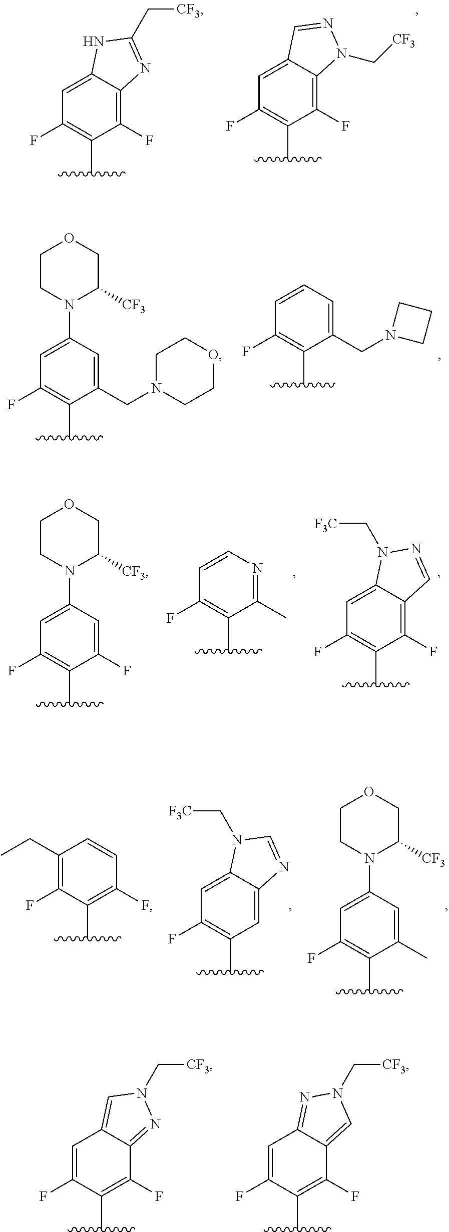

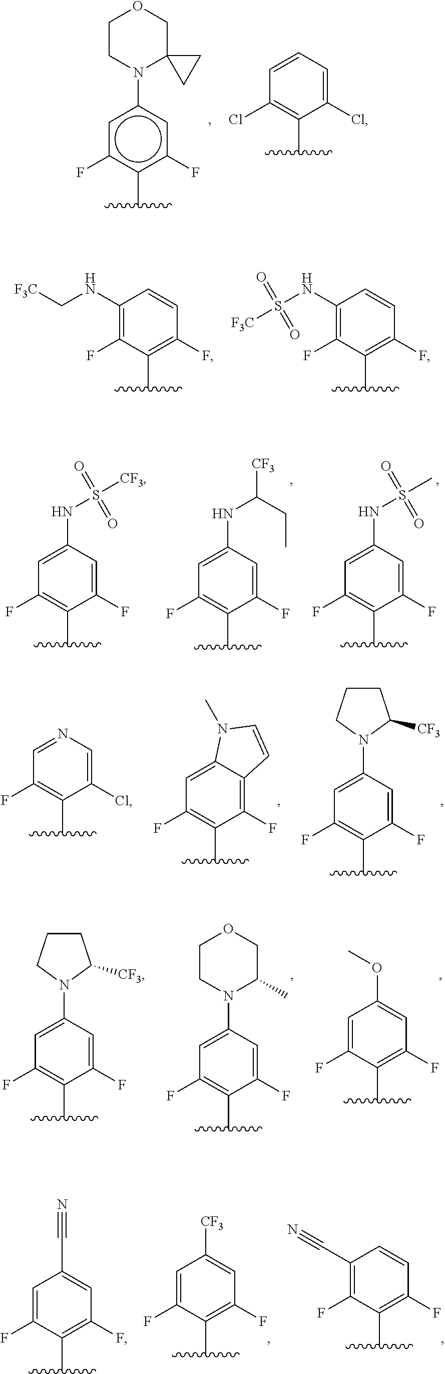

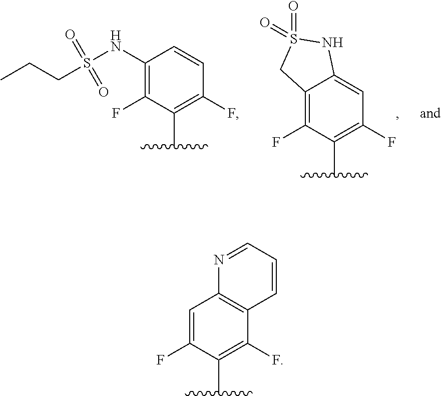 [SMILES: *c1c(F)ccc(NS(=O)(=O)CCC)c1F ; *c1c(F)cc2c(c1F)CS(=O)(=O)N2 ; *c1c(F)cc2ncccc2c1F]

In some embodiments, for the compound or salt of Formulas (I), (I-a), (II) or (III), B is selected from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

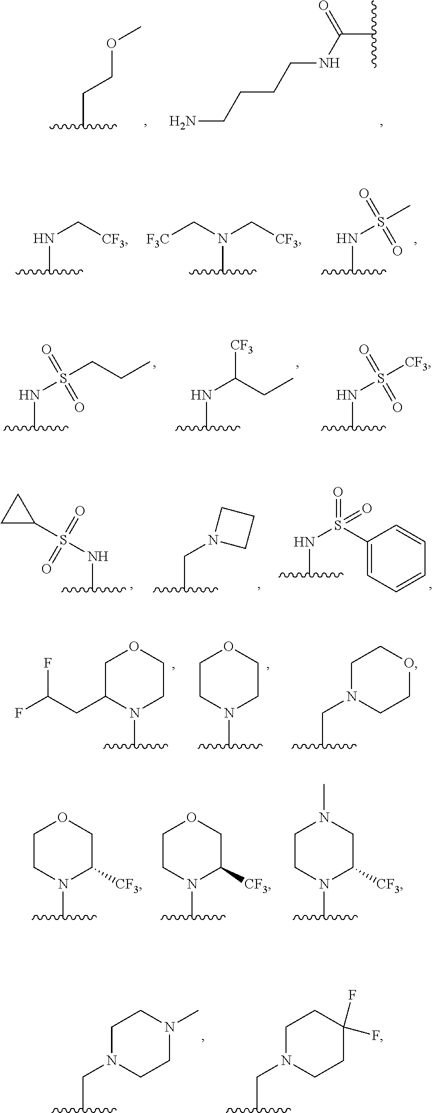

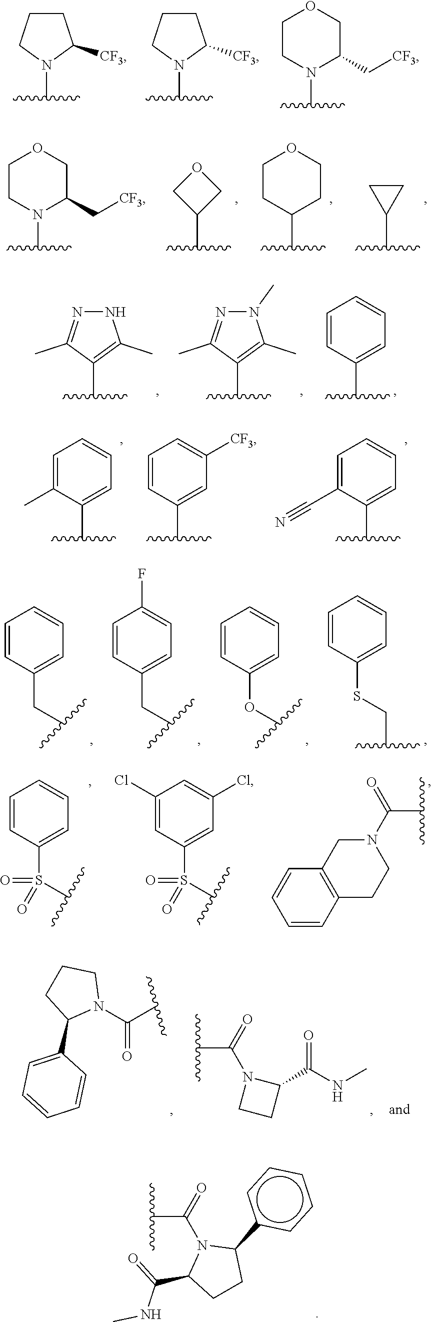

In some embodiments, for the compound or salt of Formulas (I), (I-a), (II) or (III), C is selected from C₅₋₁₀ carbocycle and 5- to 10-membered heterocycle, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, —S, ═NR¹⁸, —N₃, and —CN.

In some embodiments, for the compound or salt of Formulas (I), (I-a), (II) or (III), C is C₅₋₁₀ carbocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle, wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is 5- to 10-membered heterocycle, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, —S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, —S, ═NR¹⁸, —N₃, —CN, C₃₋₆ carbocycle and 3- to 6-membered heterocycle, wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is selected from C₅₋₁₀ carbocycle and 5- to 10-membered heterocycle, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)R¹⁸, —S(O)₂R¹⁸, —S(O)₂N(R¹⁸)₂, —NO₂, —O, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)R¹⁸, —S(O)₂R¹⁸, —S(O)₂N(R¹⁸)₂, —NO₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, —NO₂, —O, and —CN.

In some embodiments, for the compound or salt of Formulas (I), (I-a), (II) or (III), C is selected from C₅₋₁₀ carbocycle and 5- to 10-membered heterocycle
halogen, —OR¹⁸, —SR¹⁸, —N(R¹⁸)₂, —C(O)R¹⁸, —C(O)OR¹⁸, —C(O)N(R¹⁸)₂, —S(O)R¹⁸, —S(O)₂R¹⁸, —NO₂, ═O, ═S, and —CN;
C₁₋₆ alkyl and C₂₋₆ alkenyl, each of which is optionally substituted with one or more substituents independently selected from: halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)₂N(R¹⁸)₂, —NO₂, ═O, ═S, —CN, and 3- to 6-membered heterocycle optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from C₅₋₁₀ carbocycle and 5- to 10-membered heterocycle, each of which is optionally substituted with one or more substituents independently selected from:
halogen, —OR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, —O, and —CN;
C₁₋₆ alkyl, C₂₋₆ alkenyl, and C₂₋₆ alkynyl, each of which is optionally substituted with one or more substituents independently selected from:
   halogen, —OR¹⁸, —N(R¹⁸)₂, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, ═O, and —CN.

In some embodiments, for the compound or salt of Formulas (I), (I-a), (II) or (III), C is selected from C₅₋₁₀ carbocycle and 5- to 10-membered heterocycle each of which is optionally substituted with one or more substituents independently selected from:
halogen, —OR¹⁸, —N(R¹⁸)₂, ═O, and —CN;
C₁₋₆ alkyl and C₂₋₆ alkenyl, each of which is optionally substituted with one or more substituents independently selected from: halogen, —OR¹⁸, —N(R¹⁸)₂, ═O, —CN, and 3- to 6-membered heterocycle optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from 5- to 10-membered heterocycle optionally substituted with one or more substituents independently selected from:
—OR¹⁸, —N(R¹⁸)₂, and ═O;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), R¹⁸ is independently selected at each occurrence from hydrogen, C₁₋₆ alkyl, C₁₋₆ haloalkyl, and C₁₋₆ hydroxyalkyl. In some embodiments, R¹⁸ is independently selected at each occurrence from hydrogen, C₁₋₆ alkyl, and C₁₋₆ haloalkyl. In some embodiments, R¹⁸ is independently selected at each occurrence from hydrogen and C₁₋₆ alkyl. In some embodiments, R¹⁸ is independently selected at each occurrence from C₁₋₆ alkyl. In some embodiments, each R¹⁸ is hydrogen.

In some embodiments, for the compound or salt of Formulas (I), (I-a), (II) or (III), C is selected from C₅₋₁₀ carbocycle and 5- to 10-membered heterocycle each of which is optionally substituted with one or more substituents independently selected from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl,

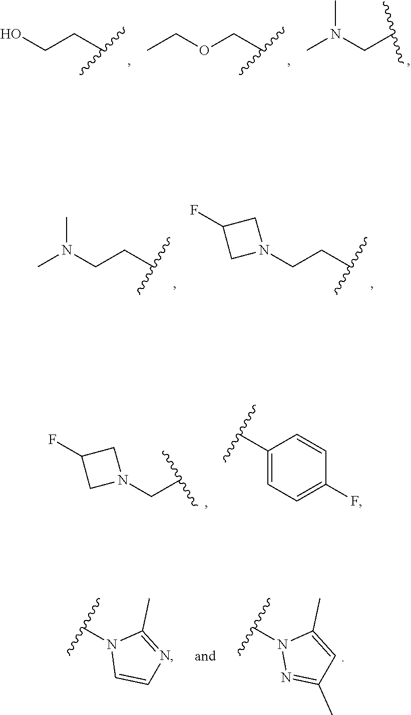

In some embodiments, for the compound or salt of Formulas (I), (I-a), (II) or (III), C is selected from C₃₋₆ monocyclic carbocycle, 5-6-membered monocyclic heterocycle, and 7-10-membered bicyclic heterocycle, each of which is optionally substituted. In some embodiments, C is selected from phenyl, 5-6-membered monocyclic heterocycle, and 7-10-membered bicyclic heterocycle, each of which is optionally substituted. In some embodiments, C is selected from phenyl, 5-6-membered monocyclic heterocycle, and 7-10-membered bicyclic heterocycle, each of which is optionally substituted. In some embodiments, C is selected from C₃₋₆ monocyclic carbocycle and 5-6-membered monocyclic heterocycle each of which is optionally substituted. In some embodiments, C is selected from phenyl and 5-6-membered monocyclic heterocycle each of which is optionally substituted. In some embodiments, C is selected from 7-10-membered bicyclic heterocycle, each of which is optionally substituted.

In some embodiments, for the compound or salt of Formulas (I), (I-a), (II) or (III), C is selected from phenyl, pyridine, pyridin-2-one, pyrimidone, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted. In some embodiments, C is selected from phenyl, pyridine, pyridin-2-one, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted. In some embodiments, C is selected from phenyl, pyridine, pyridin-2-one, pyrimidinedione, pyrazine, and pyridazin-3-one, each of which is optionally substituted. In some embodiments, C is selected from isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted.

In some embodiments, for the compound or salt of Formulas (I), (I-a), (II) or (III), the optional substituents on C are independently selected at each occurrence from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), the optional substituents on C are independently selected at each occurrence from:
halogen, —OR¹⁸, —SR¹⁸, —N(R¹⁸)₂, —C(O)R¹⁸, —C(O)OR¹⁸, —C(O)N(R¹⁸)₂, —S(O)R¹⁸, —S(O)₂R¹⁸, —NO₂, ═O, ═S, and —CN;
C₁₋₆ alkyl and C₂₋₆ alkenyl, each of which is optionally substituted with one or more substituents independently selected from: halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)₂N(R¹⁸)₂, —NO₂, ═O, ═S, —CN, and 3- to 6-membered heterocycle optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), the optional substituents on C are independently selected at each occurrence from:
halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)R¹⁸, —S(O)₂R¹⁸, —S(O)₂N(R¹⁸)₂, —NO₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)R¹⁸, —S(O)₂R¹⁸, —S(O)₂N(R¹⁸)₂, —NO₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, —NO₂, ═O, and —CN.

In some embodiments, for the compound or salt of Formulas (I), (I-a), (II) or (III), the optional substituents on C are independently selected at each occurrence from:
halogen, —OR¹⁸, —N(R¹⁸)₂, —O, and —CN;
C₁₋₆ alkyl and C₂₋₆ alkenyl, each of which is optionally substituted with one or more substituents independently selected from: halogen, —OR¹⁸, —N(R¹⁸)₂, ═O, —CN, and 3- to 6-membered heterocycle optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), the optional substituents on C are independently selected at each occurrence from:
—OR¹⁸, —N(R¹⁸)₂, and =0;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), the optional substituents on C are independently selected at each occurrence from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl

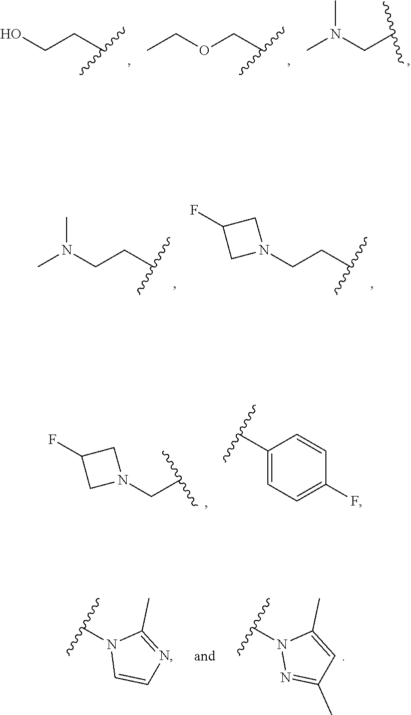

In some embodiments, for the compound or salt of Formulas (I), (I-a), (II) or (III), C is selected from phenyl, pyridine, pyridin-2-one, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is selected from phenyl, pyridine, pyridin-2-one, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from:
halogen, —OR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁸, —N(R¹⁸)₂, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, ═O, and —CN.

In some embodiments, for the compound or salt of Formulas (I), (I-a), (II) or (III), C is selected from phenyl, pyridine, pyridin-2-one, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from:
—OR¹⁸, —N(R¹⁸)₂, and ═O;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from phenyl, pyridine, pyridin-2-one, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from: halogen, ═O, —N(CH₃)₂. C₁₋₆ alkyl, C₂₋₆ alkenyl, C₁₋₆ haloalkyl, —O—C₁₋₆ alkyl, —O—C₁₋₆ haloalkyl,

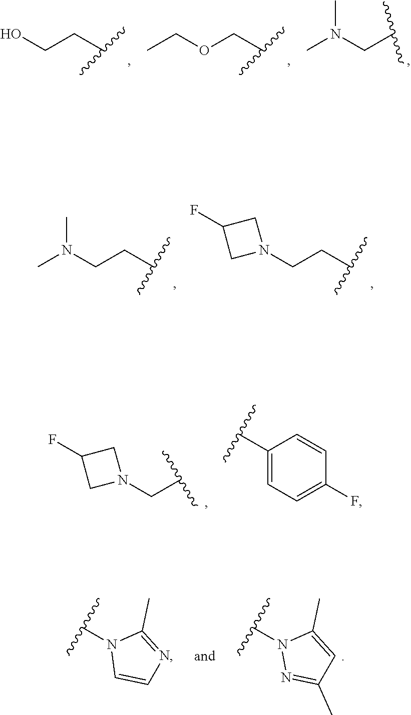

In some embodiments, for the compound or salt of Formulas (I), (I-a), (II) or (III), C is selected from C₃₋₆ monocyclic carbocycle and 5-6-membered monocyclic heterocycle each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is selected from C₃₋₆ monocyclic carbocycle and 5-6-membered monocyclic heterocycle each of which is optionally substituted with one or more substituents independently selected from:
halogen, —OR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁸, —N(R¹⁸)₂, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, ═O, and —CN.

In some embodiments, for the compound or salt of Formulas (I), (I-a), (II) or (III), C is selected from C₃₋₆ monocyclic carbocycle and 5-6-membered monocyclic heterocycle each of which is optionally substituted with one or more substituents independently selected from:
—OR¹⁸, —N(R¹⁸)₂, and ═O;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from C₃₋₆ monocyclic carbocycle and 5-6-membered monocyclic heterocycle each of which is optionally substituted with one or more substituents independently selected from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl,

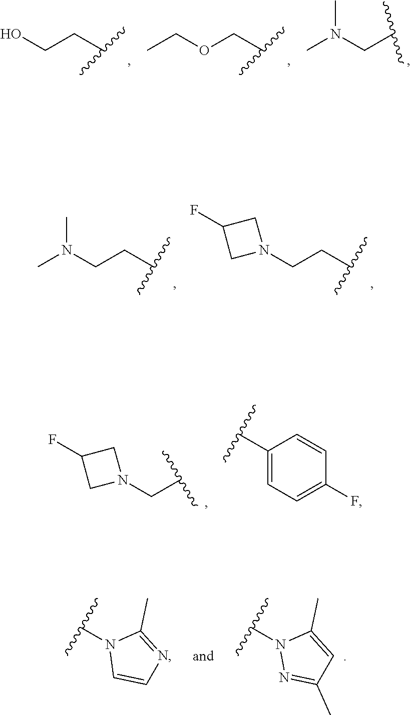

In some embodiments, for the compound or salt of Formulas (I), (I-a), (II) or (III), C is selected from phenyl, pyridine, pyridin-2-one, pyrimidinedione, pyrazine, and pyridazin-3-one, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O) OR¹⁸, —N(R¹⁸)C(O) N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O) OR¹⁸, —N(R¹⁸)C(O) N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is selected from phenyl, pyridine, pyridin-2-one, pyrimidinedione, pyrazine, and pyridazin-3-one, each of which is optionally substituted with one or more substituents independently selected from:
halogen, —OR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁸, —N(R¹⁸)₂, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, ═O, and —CN.

In some embodiments, for the compound or salt of Formulas (I), (I-a), (II) or (III), C is selected from phenyl, pyridine, pyridin-2-one, pyrimidinedione, pyrazine, and pyridazin-3-one, each of which is optionally substituted with one or more substituents independently selected from:
—OR¹⁸, —N(R¹⁸)₂, and ═O;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from phenyl, pyridine, pyridin-2-one, pyrimidinedione, pyrazine, and pyridazin-3-one, each of which is optionally substituted with one or more substituents independently selected from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl,

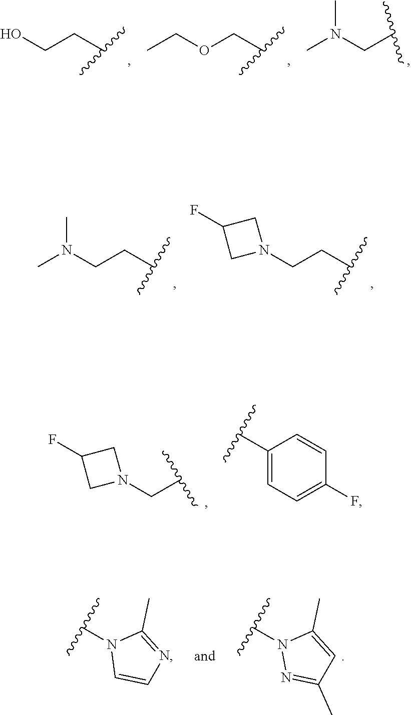

In some embodiments, for the compound or salt of Formulas (I), (I-a), (II) or (III), C is selected from 7-10-membered bicyclic heterocycle, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O) OR¹⁸, —N(R¹⁸)C(O) N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O) OR¹⁸, —N(R¹⁸)C(O) N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is selected from 7-10-membered bicyclic heterocycle, each of which is optionally substituted with one or more substituents independently selected from:
halogen, —OR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, —O, and —CN;
C₁₋₆ alkyl, C₂₋₆ alkenyl, and C₂₋₆ alkynyl, each of which is optionally substituted with one or more substituents independently selected from:
   halogen, —OR¹⁸, —N(R¹⁸)₂, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, —O, and —CN.

In some embodiments, for the compound or salt of Formulas (I), (I-a), (II) or (III), C is selected from 7-10-membered bicyclic heterocycle, each of which is optionally substituted with one or more substituents independently selected from:
—OR¹⁸, —N(R¹⁸)₂, and ═O;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from 7-10-membered bicyclic heterocycle, each of which is optionally substituted with one or more substituents independently selected from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl,

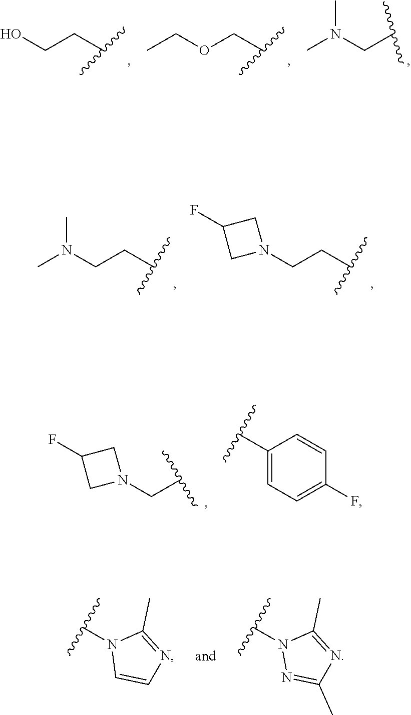

In some embodiments, for the compound or salt of Formulas (I), (I-a), (II) or (III), C is selected from isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O) OR¹⁸, —N(R¹⁸)C(O) N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, —S, ═NR¹⁸, —N₃, and —CN;
C₁₋₆ alkyl, C₂₋₆ alkenyl, and C₂₋₆ alkynyl, each of which is optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O) OR¹⁸, —N(R¹⁸)C(O) N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═NR¹⁸, —N₃, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN.

In some embodiments, for the compound or salt of Formulas (I), (I-a), (II) or (III), C is selected from isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from:
halogen, —OR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, and —CN;
C₁₋₆ alkyl, C₂₋₆ alkenyl, and C₂₋₆ alkynyl, each of which is optionally substituted with one or more substituents independently selected from:
   halogen, —OR¹⁸, —N(R¹⁸)₂, —C(O)OR¹⁸, —OC(O)R¹⁸, —C(O)N(R¹⁸)₂, —N(R¹⁸)C(O)R¹⁸, —N(R¹⁸)S(O)₂(R¹⁸), —S(O)₂N(R¹⁸)₂, ═O, —CN, C₃₋₆ carbocycle and 3- to 6-membered heterocycle,
      wherein the C₃₋₆ carbocycle and 3- to 6-membered heterocycle are optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —N(R¹⁸)₂, —C(O)R¹⁸, —O, and —CN.

In some embodiments, for the compound or salt of Formulas (I), (I-a), (II) or (III), C is selected from isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from:
—OR¹⁸, —N(R¹⁸)₂, and ═O;
C₁₋₆ alkyl and C₂₋₆ alkenyl, each of which is optionally substituted with one or more substituents independently selected from: halogen, —OR¹⁸, —N(R¹⁸)₂, and 3- to 6-membered heterocycle optionally substituted with one or more substituents independently selected from: halogen; and
C₃₋₆ carbocycle and 3- to 6-membered heterocycle, each of which is optionally substituted with one or more substituents independently selected from halogen, C₁₋₆ alkyl, and C₁₋₆ haloalkyl.

In some embodiments, for the compound or salt of Formulas (I), (I-a), (II) or (III), C is selected from isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted with one or more substituents independently selected from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl,

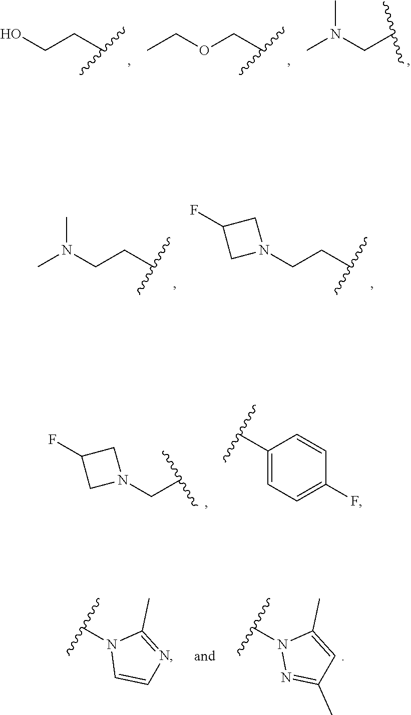

In some embodiments, for the compound or salt of Formulas (I), (I-a), (II) or (III), C is selected from

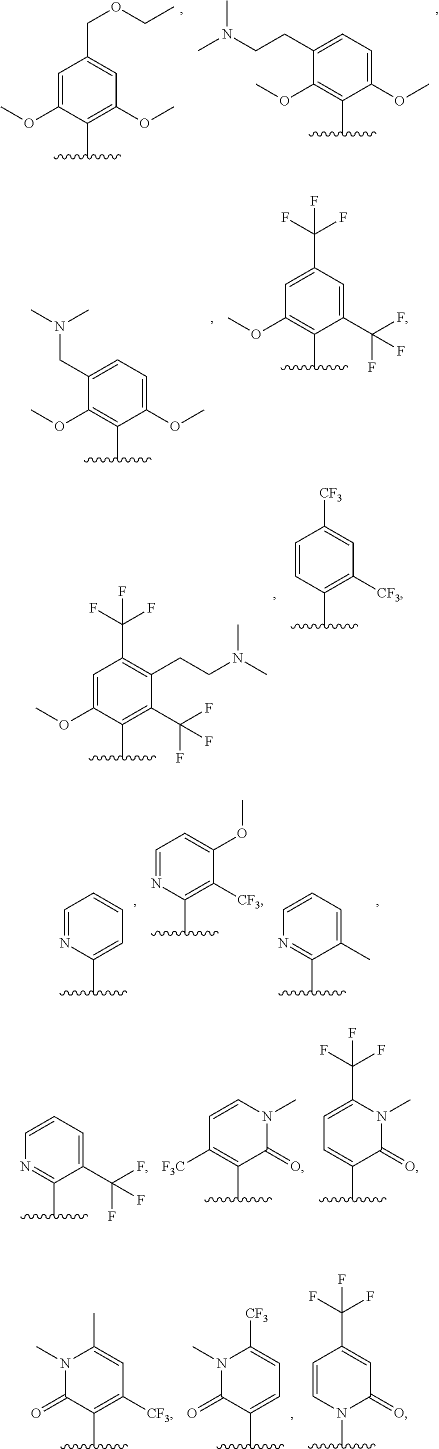

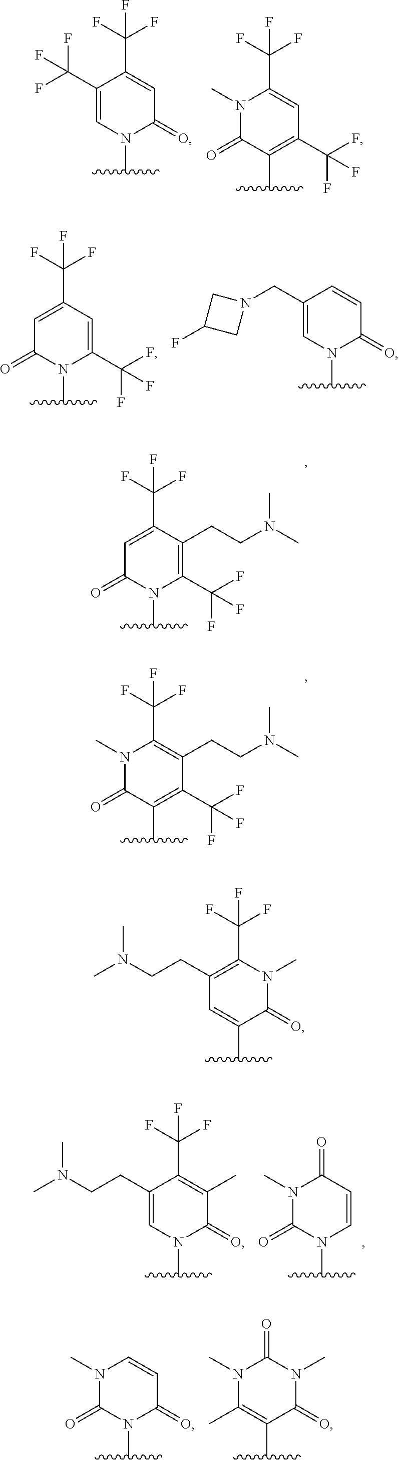

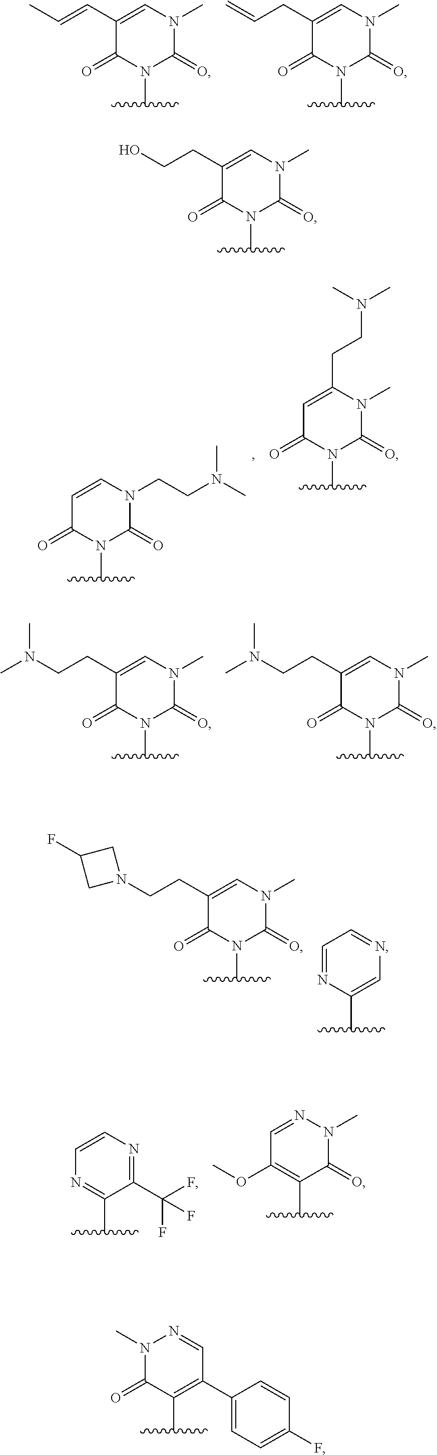

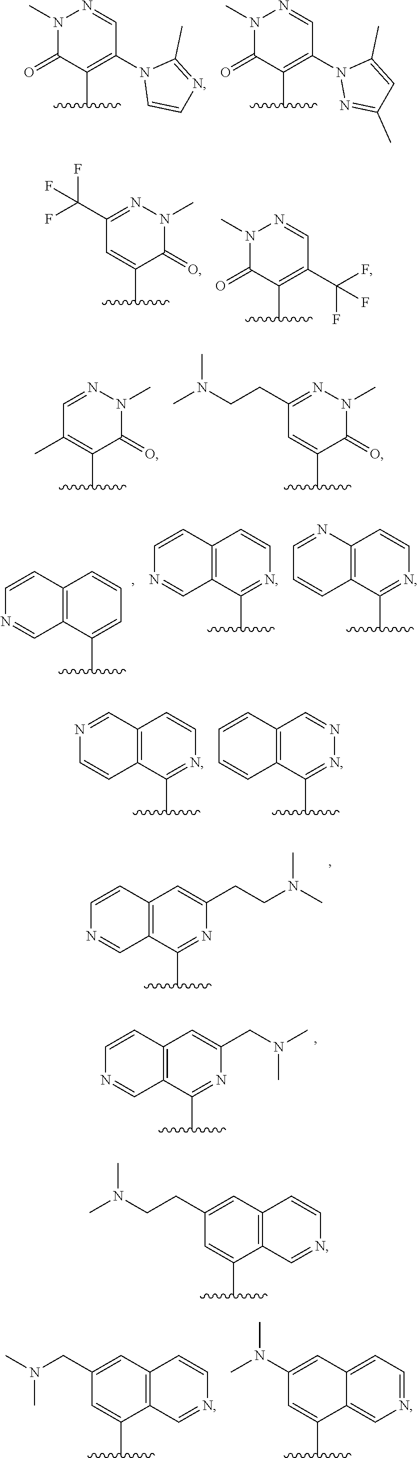

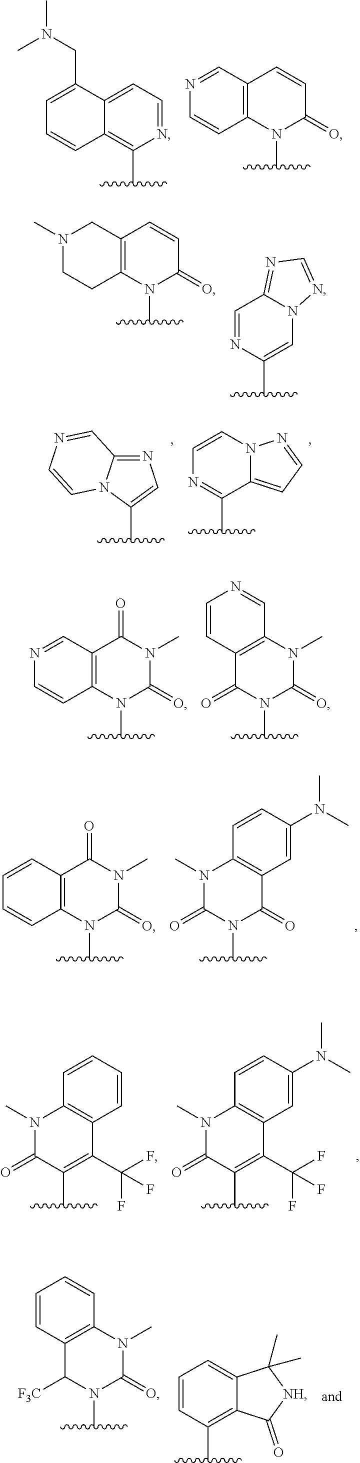

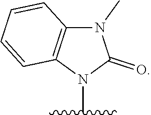 [SMILES: *n1c(=O)n(C)c2ccccc21]

In some embodiments, for the compound or salt of Formulas (I), (I-a), (II) or (III), C is selected from

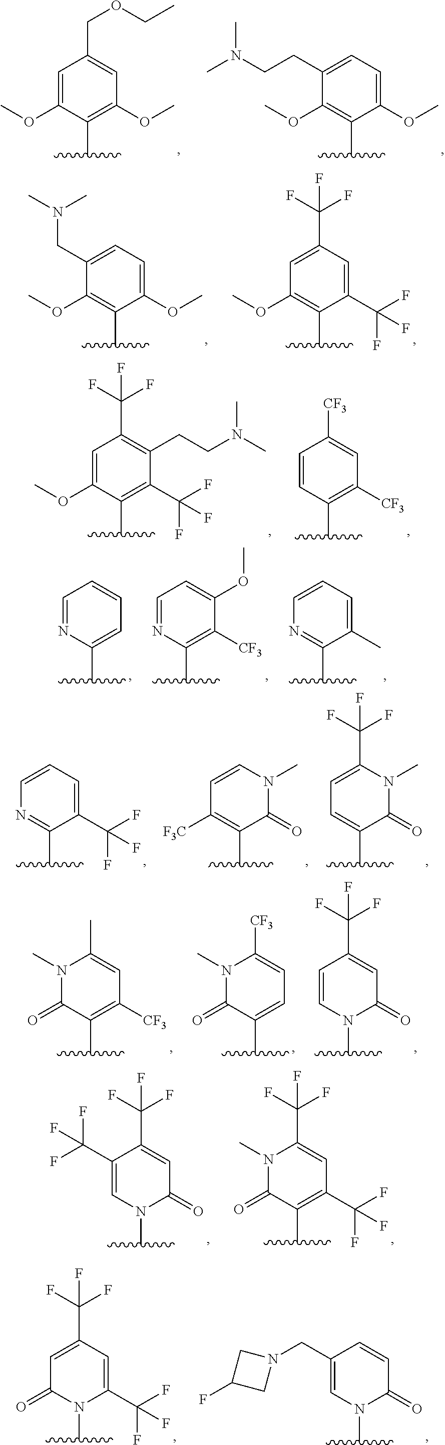

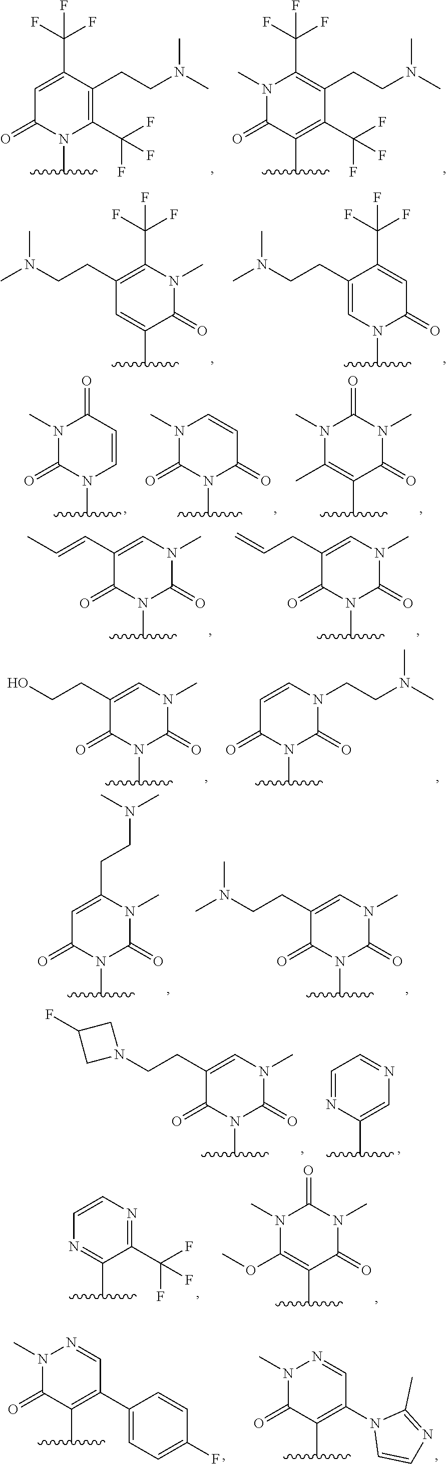

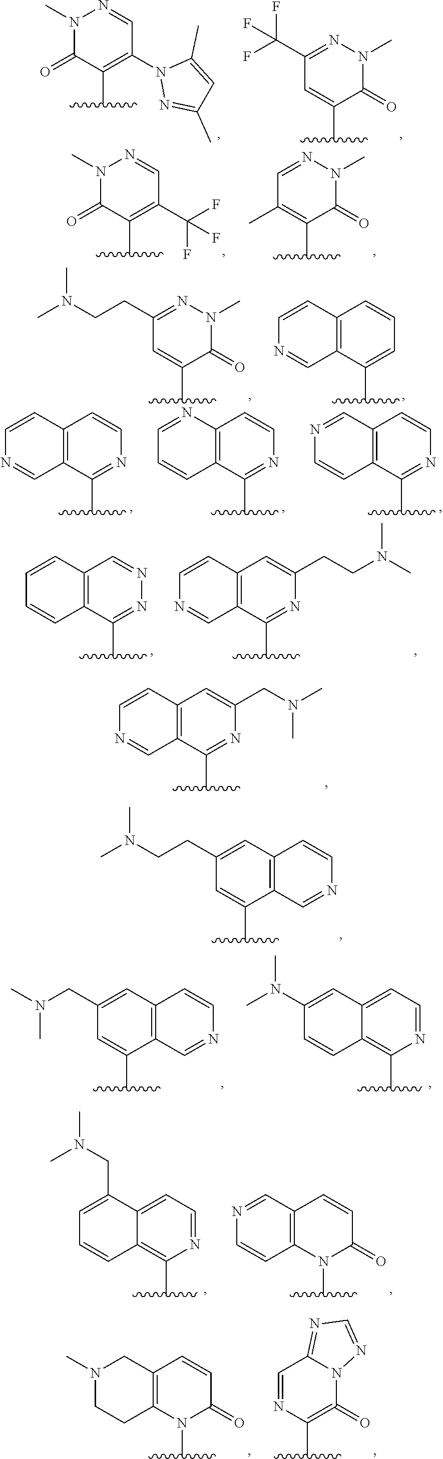

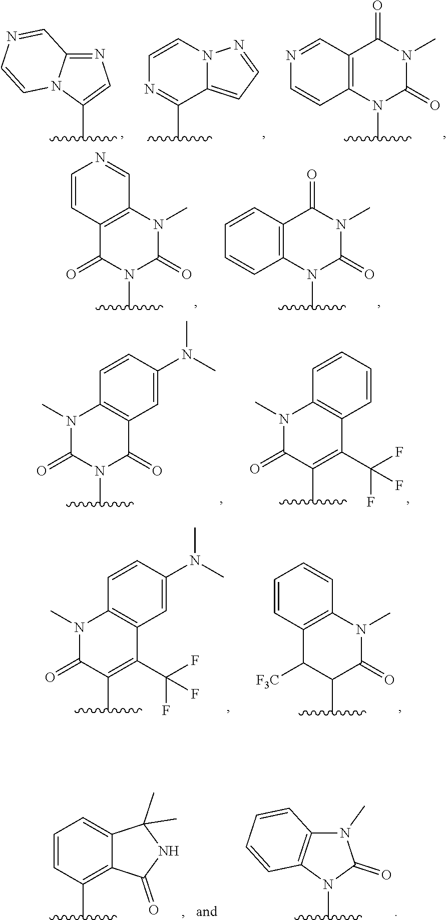

In some embodiments, for the compound or salt of Formulas (I), (I-a), (II) or (III), C is selected from:

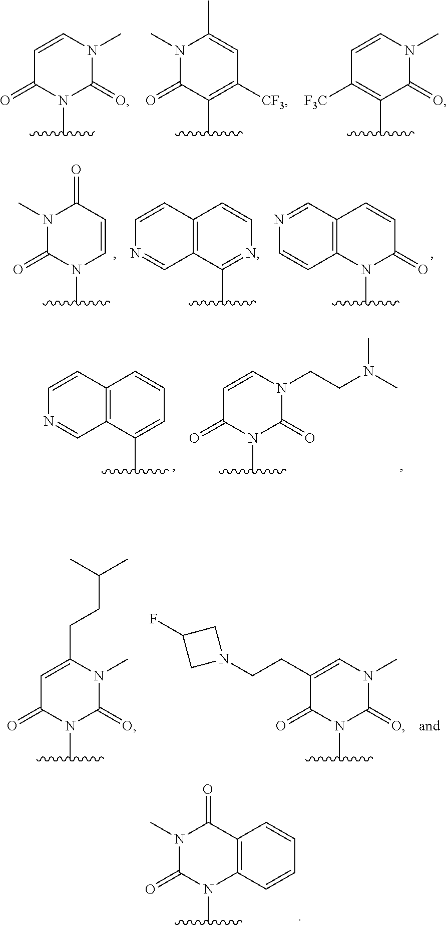

In some embodiments, for the compound or salt of Formulas (I), (I-a), (II) or (III), C is selected from:

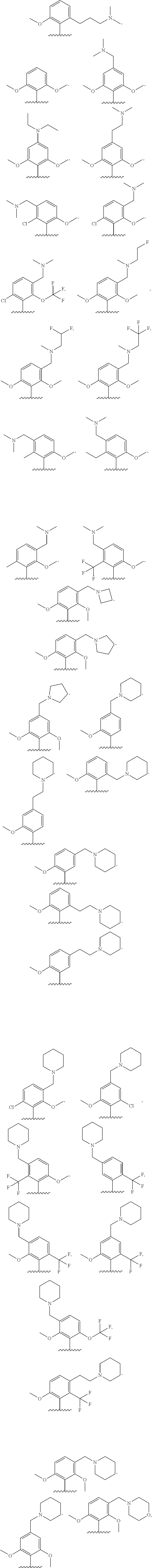

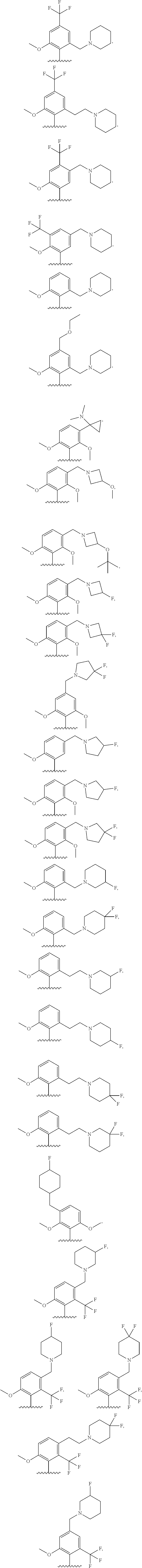

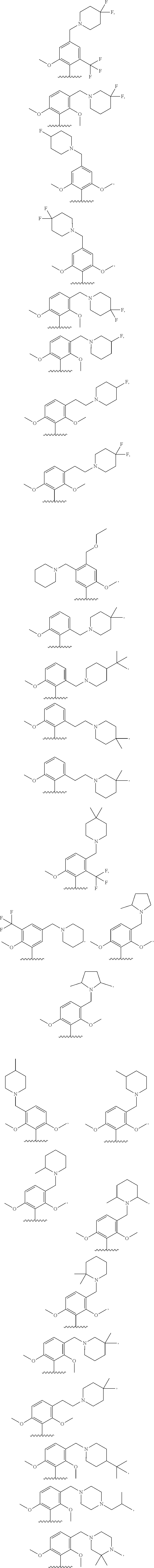

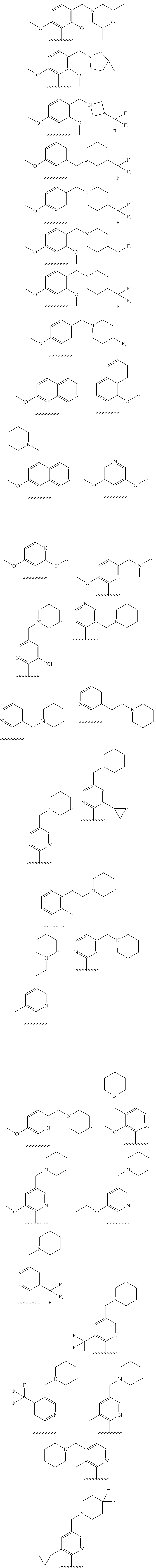

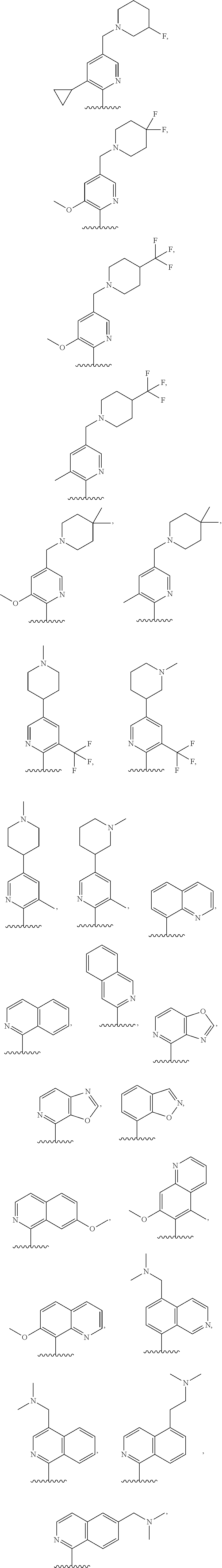

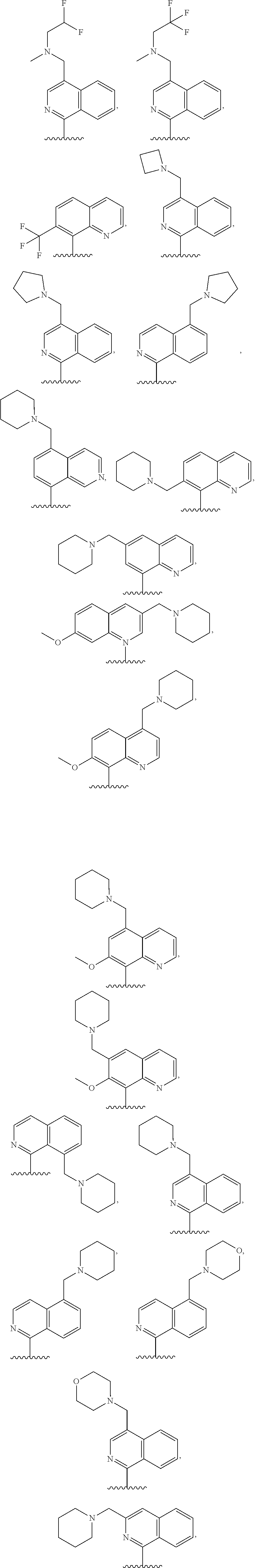

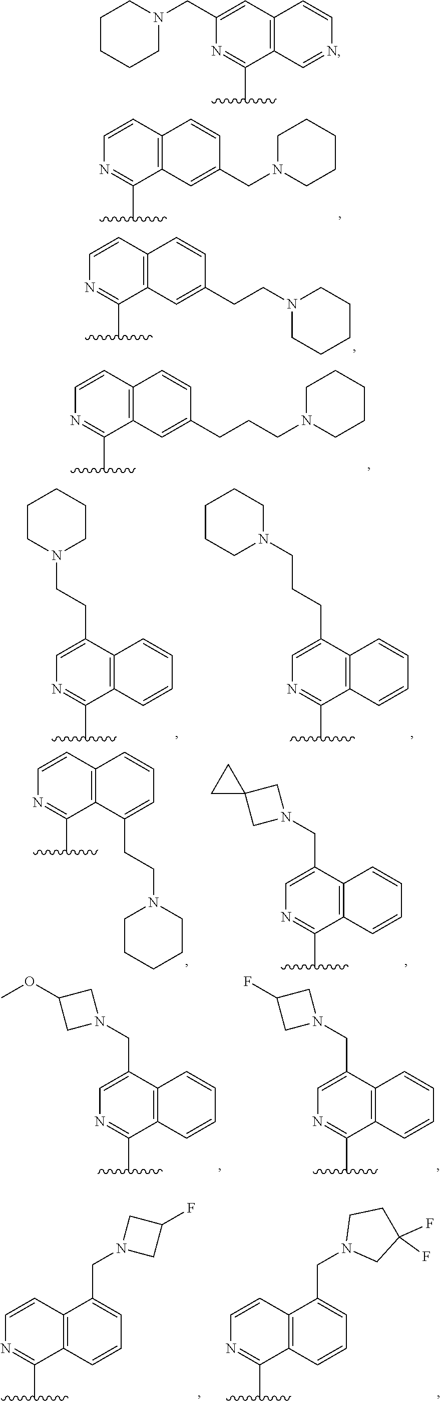

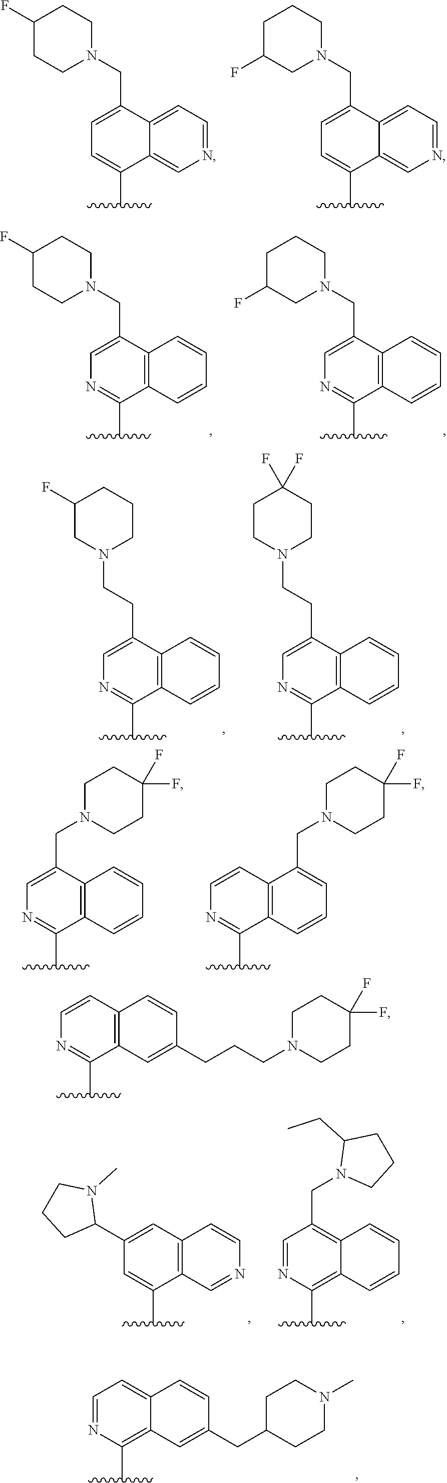

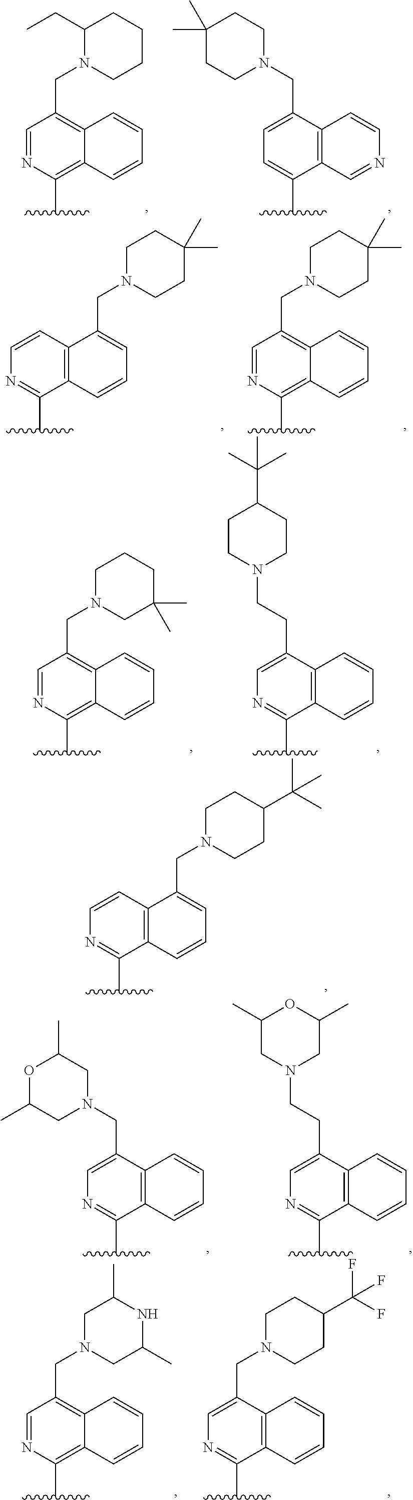

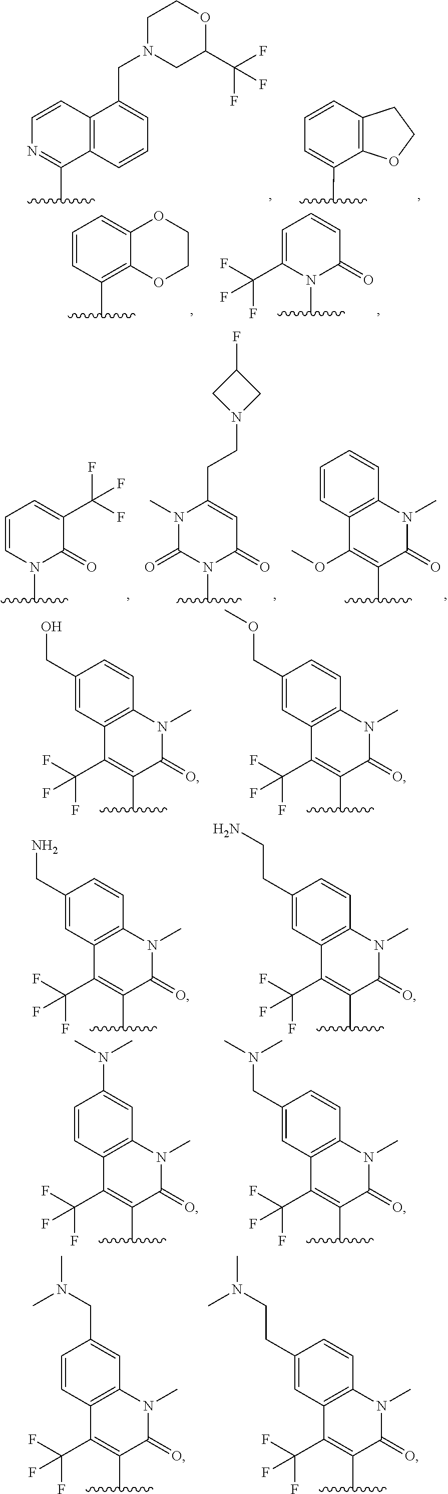

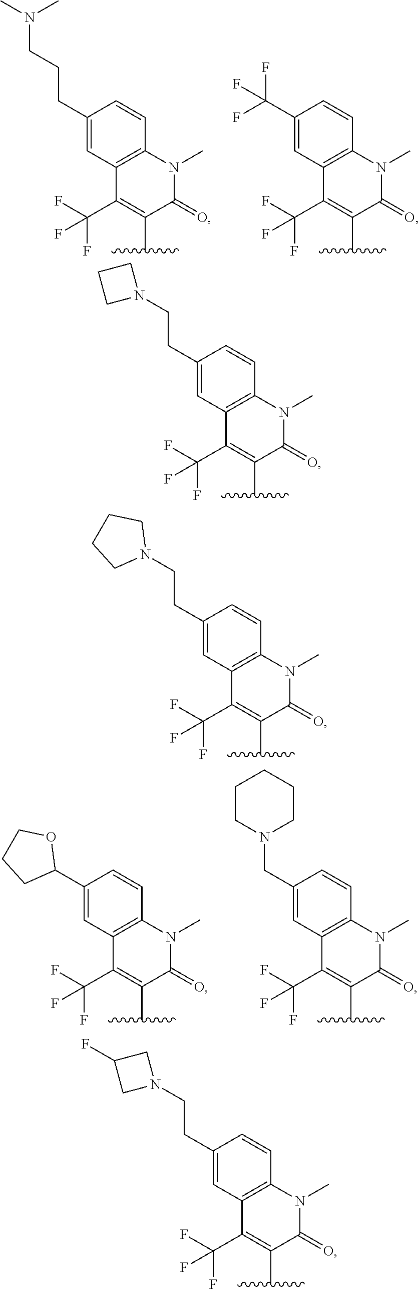

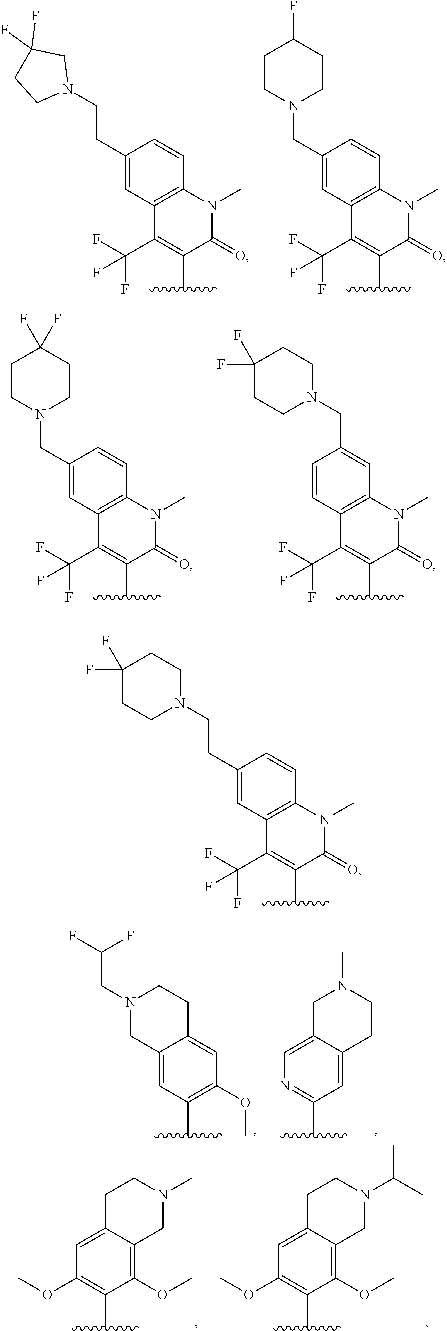

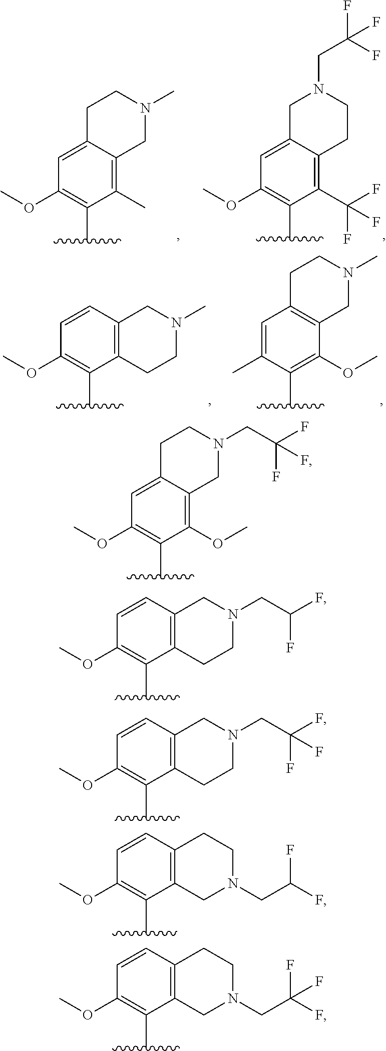

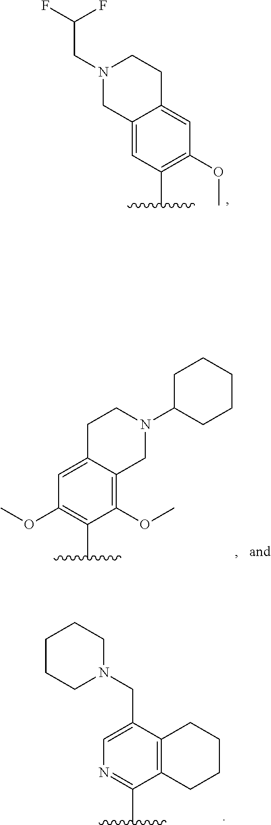

In some embodiments, for the compound or salt of Formulas (I), (I-a), (II) or (III), C is selected from:

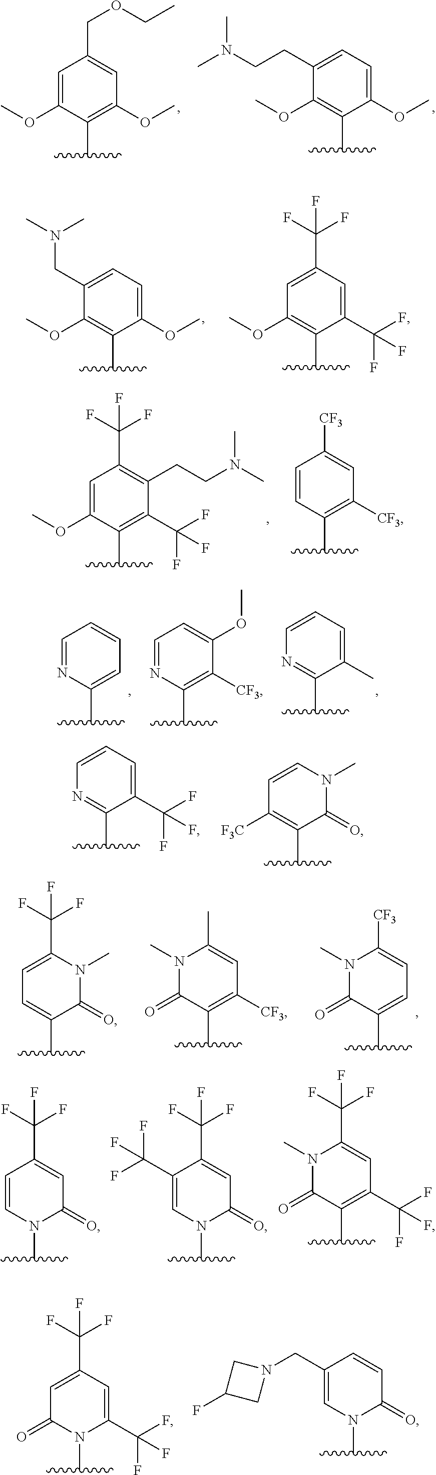

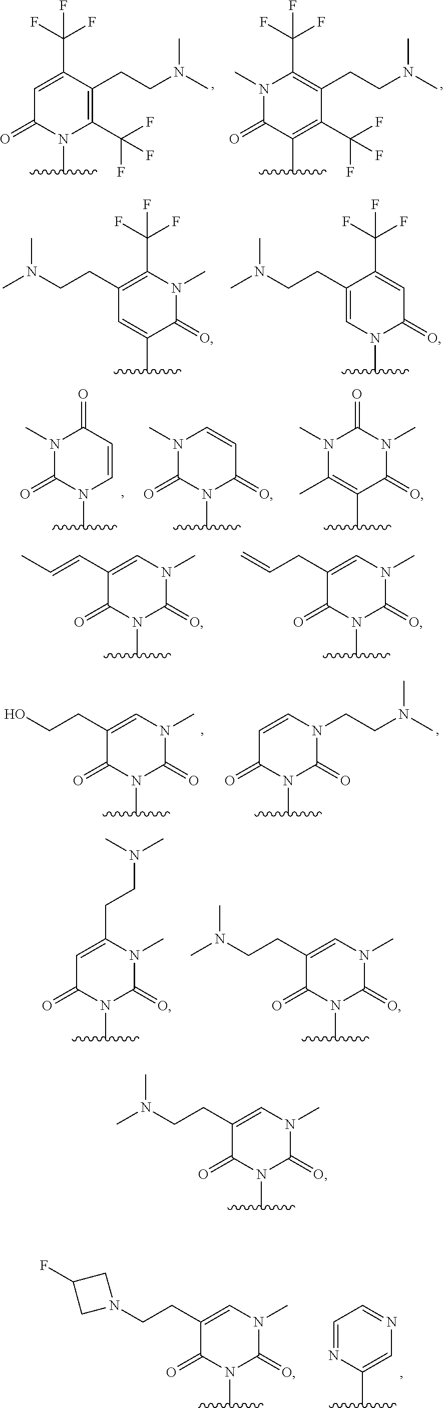

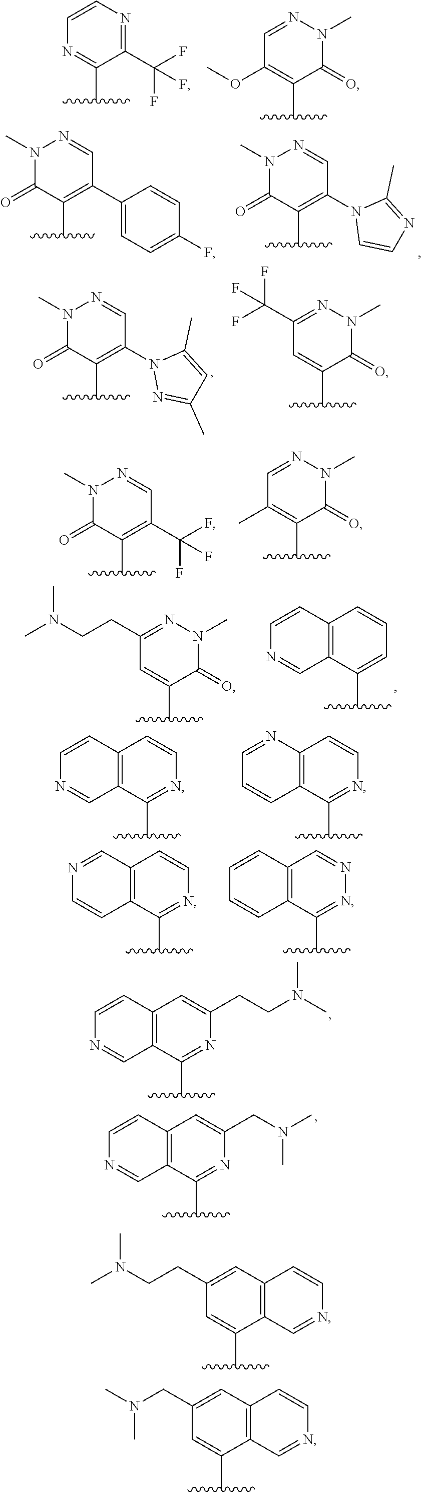

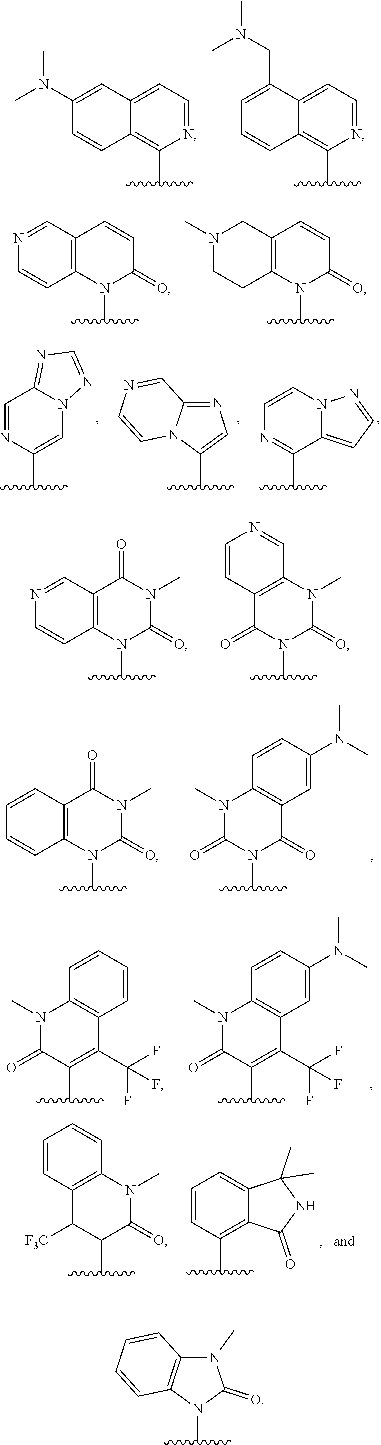

In some embodiments, the compound or salt of Formulas (I), (I-a), (II) or (III) is selected from Table 1, below, or a pharmaceutically acceptable salt thereof.

TABLE 1

| Compound | |
|---|---|
| No. | Structure |
| 1 | 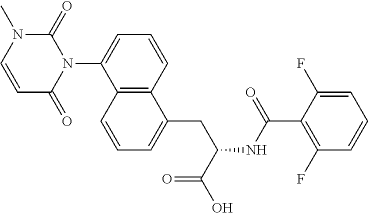 [SMILES: Cn1ccc(=O)n(-c2cccc3c(C[C@H](NC(=O)c4c(F)cccc4F)C(=O)O)cccc23)c1=O] |
| 2 | 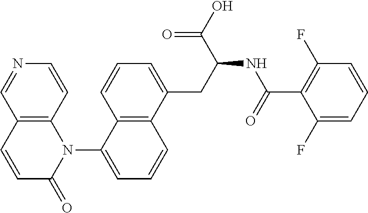 [SMILES: O=C(N[C@@H](Cc1cccc2c(-n3c(=O)ccc4cnccc43)cccc12)C(=O)O)c1c(F)cccc1F] |
| 3 | 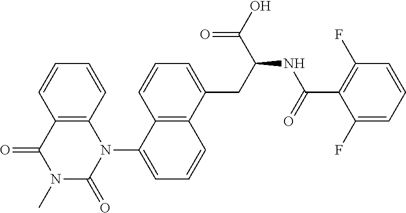 [SMILES: Cn1c(=O)c2ccccc2n(-c2cccc3c(C[C@H](NC(=O)c4c(F)cccc4F)C(=O)O)cccc23)c1=O] |
| 4 | 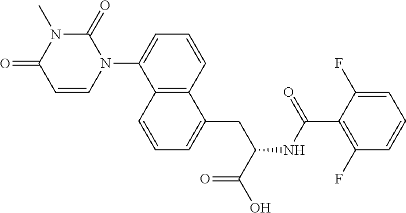 [SMILES: Cn1c(=O)ccn(-c2cccc3c(C[C@H](NC(=O)c4c(F)cccc4F)C(=O)O)cccc23)c1=O] |
| 5 | 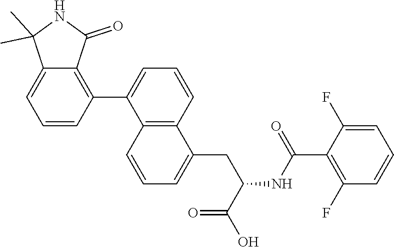 [SMILES: CC1(C)NC(=O)c2c(-c3cccc4c(C[C@H](NC(=O)c5c(F)cccc5F)C(=O)O)cccc34)cccc21] |
| 6 | 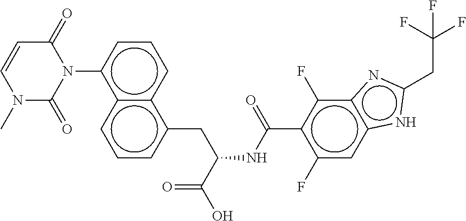 |
| 7 | 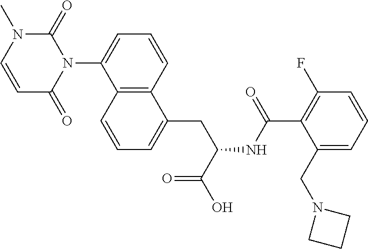 [SMILES: Cn1ccc(=O)n(-c2cccc3c(C[C@H](NC(=O)c4c(F)cccc4CN4CCC4)C(=O)O)cccc23)c1=O] |
| 8 | 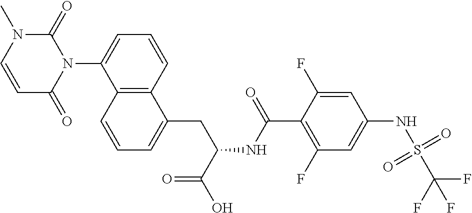 [SMILES: Cn1ccc(=O)n(-c2cccc3c(C[C@H](NC(=O)c4c(F)cc(NS(=O)(=O)C(F)(F)F)cc4F)C(=O)O)cccc23)c1=O] |
| 9 | 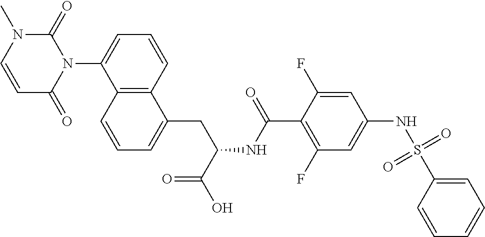 [SMILES: Cn1ccc(=O)n(-c2cccc3c(C[C@H](NC(=O)c4c(F)cc(NS(=O)(=O)c5ccccc5)cc4F)C(=O)O)cccc23)c1=O] |
| 10 | 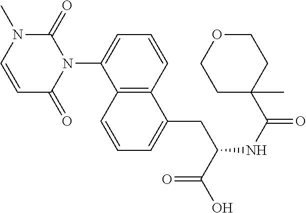 [SMILES: Cn1ccc(=O)n(-c2cccc3c(C[C@H](NC(=O)C4(C)CCOCC4)C(=O)O)cccc23)c1=O] |
| 11 | 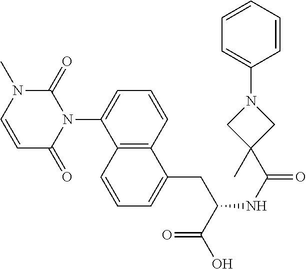 [SMILES: Cn1ccc(=O)n(-c2cccc3c(C[C@H](NC(=O)C4(C)CN(c5ccccc5)C4)C(=O)O)cccc23)c1=O] |
| 12 | 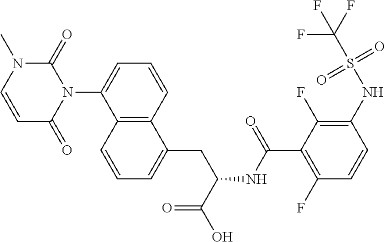 [SMILES: Cn1ccc(=O)n(-c2cccc3c(C[C@H](NC(=O)c4c(F)ccc(NS(=O)(=O)C(F)(F)F)c4F)C(=O)O)cccc23)c1=O] |
| 13 | 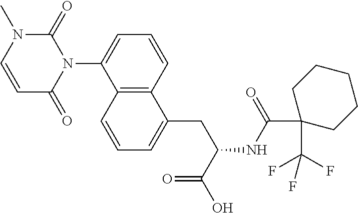 [SMILES: Cn1ccc(=O)n(-c2cccc3c(C[C@H](NC(=O)C4(C(F)(F)F)CCCCC4)C(=O)O)cccc23)c1=O] |
| 14 | 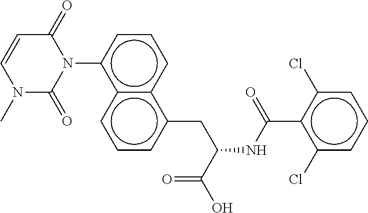 |
| 15 | 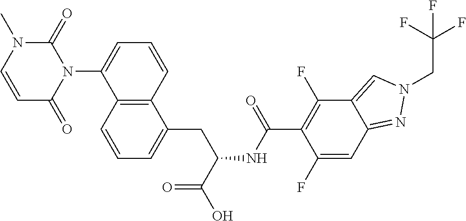 [SMILES: Cn1ccc(=O)n(-c2cccc3c(C[C@H](NC(=O)c4c(F)cc5nn(CC(F)(F)F)cc5c4F)C(=O)O)cccc23)c1=O] |
| 16 | 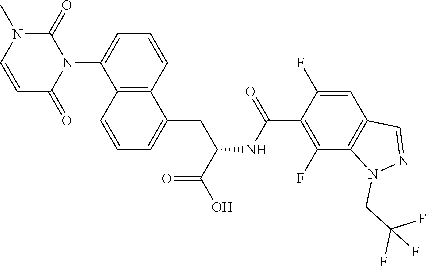 [SMILES: Cn1ccc(=O)n(-c2cccc3c(C[C@H](NC(=O)c4c(F)cc5cnn(CC(F)(F)F)c5c4F)C(=O)O)cccc23)c1=O] |
| 17 | 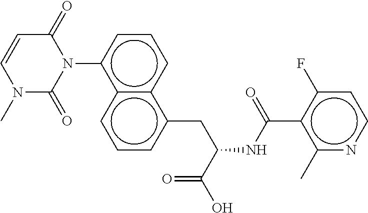 |
| 18 | 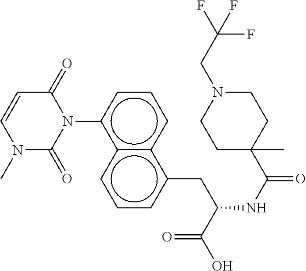 |
| 19 | 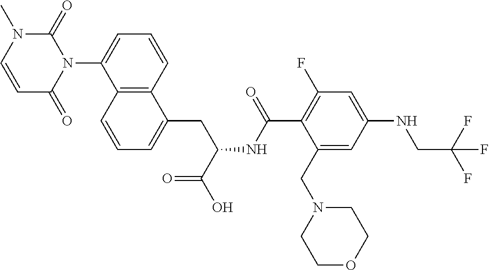 [SMILES: Cn1ccc(=O)n(-c2cccc3c(C[C@H](NC(=O)c4c(F)cc(NCC(F)(F)F)cc4CN4CCOCC4)C(=O)O)cccc23)c1=O] |
| 20 | 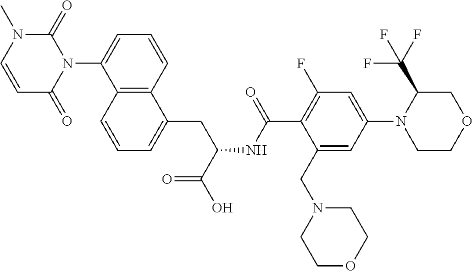 [SMILES: Cn1ccc(=O)n(-c2cccc3c(C[C@H](NC(=O)c4c(F)cc(N5CCOC[C@@H]5C(F)(F)F)cc4CN4CCOCC4)C(=O)O)cccc23)c1=O] |
| 21 | 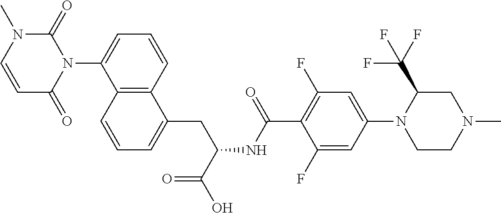 [SMILES: CN1CCN(c2cc(F)c(C(=O)N[C@@H](Cc3cccc4c(-n5c(=O)ccn(C)c5=O)cccc34)C(=O)O)c(F)c2)[C@@H](C(F)(F)F)C1] |
| 22 | 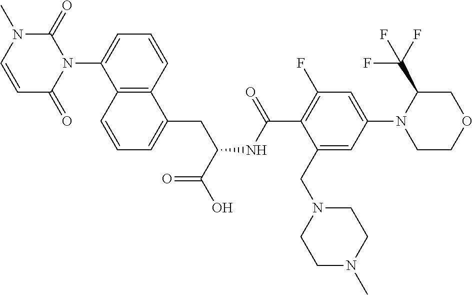 [SMILES: CN1CCN(Cc2cc(N3CCOC[C@@H]3C(F)(F)F)cc(F)c2C(=O)N[C@@H](Cc2cccc3c(-n4c(=O)ccn(C)c4=O)cccc23)C(=O)O)CC1] |
| 23 | 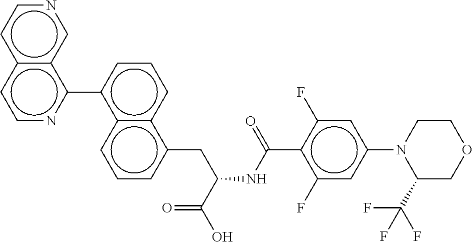 |
| 24 | 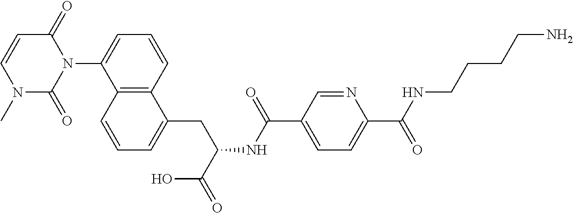 [SMILES: Cn1ccc(=O)n(-c2cccc3c(C[C@H](NC(=O)c4ccc(C(=O)NCCCCN)nc4)C(=O)O)cccc23)c1=O] |
| 25 | 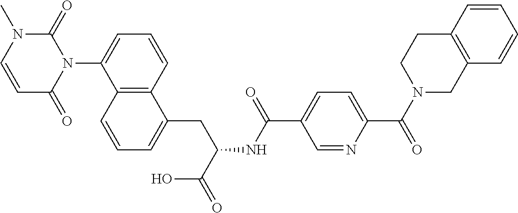 [SMILES: Cn1ccc(=O)n(-c2cccc3c(C[C@H](NC(=O)c4ccc(C(=O)N5CCc6ccccc6C5)nc4)C(=O)O)cccc23)c1=O] |
| 26 | 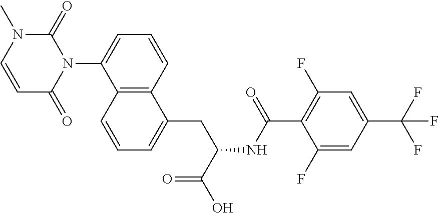 [SMILES: Cn1ccc(=O)n(-c2cccc3c(C[C@H](NC(=O)c4c(F)cc(C(F)(F)F)cc4F)C(=O)O)cccc23)c1=O] |
| 27 | 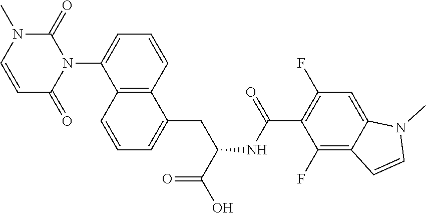 [SMILES: Cn1ccc(=O)n(-c2cccc3c(C[C@H](NC(=O)c4c(F)cc5c(ccn5C)c4F)C(=O)O)cccc23)c1=O] |
| 28 | 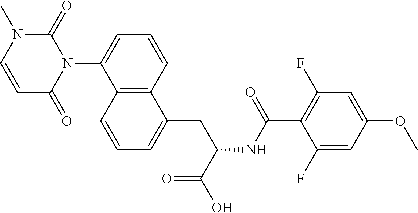 [SMILES: COc1cc(F)c(C(=O)N[C@@H](Cc2cccc3c(-n4c(=O)ccn(C)c4=O)cccc23)C(=O)O)c(F)c1] |
| 29 | 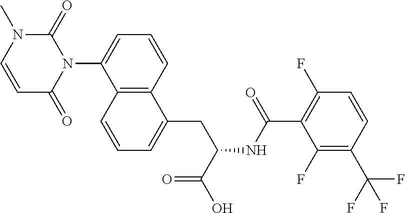 [SMILES: Cn1ccc(=O)n(-c2cccc3c(C[C@H](NC(=O)c4c(F)ccc(C(F)(F)F)c4F)C(=O)O)cccc23)c1=O] |
| 30 | 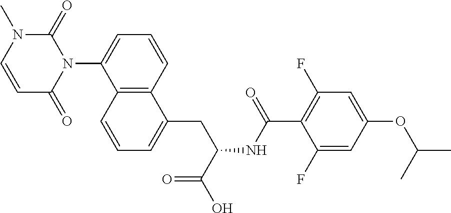 [SMILES: CC(C)Oc1cc(F)c(C(=O)N[C@@H](Cc2cccc3c(-n4c(=O)ccn(C)c4=O)cccc23)C(=O)O)c(F)c1] |
| 31 | 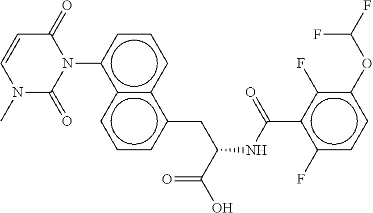 |
| 32 | 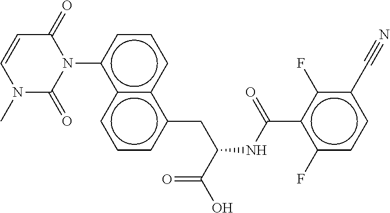 |
| 33 | 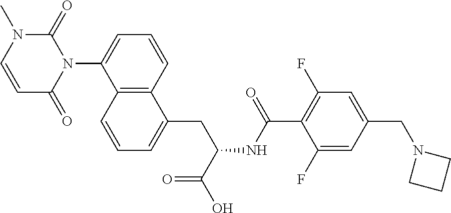 [SMILES: Cn1ccc(=O)n(-c2cccc3c(C[C@H](NC(=O)c4c(F)cc(CN5CCC5)cc4F)C(=O)O)cccc23)c1=O] |
| 34 | 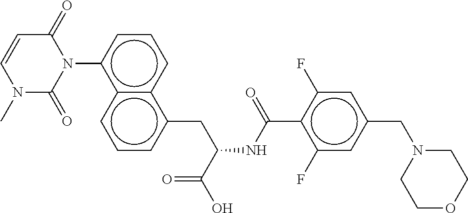 |
| 35 | 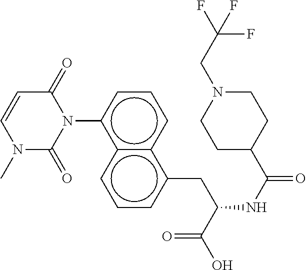 |
| 36 | 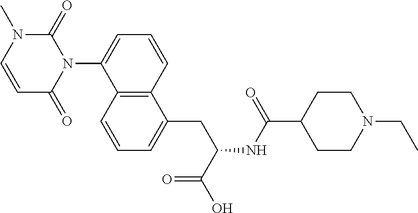 [SMILES: CCN1CCC(C(=O)N[C@@H](Cc2cccc3c(-n4c(=O)ccn(C)c4=O)cccc23)C(=O)O)CC1] |
| 37 | 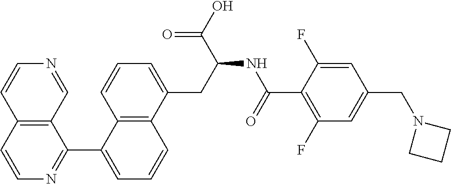 [SMILES: O=C(N[C@@H](Cc1cccc2c(-c3nccc4ccncc34)cccc12)C(=O)O)c1c(F)cc(CN2CCC2)cc1F] |
| 38 | 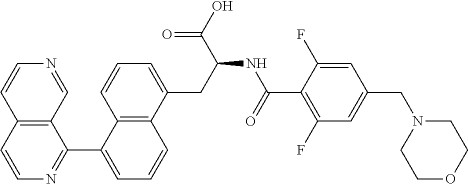 [SMILES: O=C(N[C@@H](Cc1cccc2c(-c3nccc4ccncc34)cccc12)C(=O)O)c1c(F)cc(CN2CCOCC2)cc1F] |
| 39 | 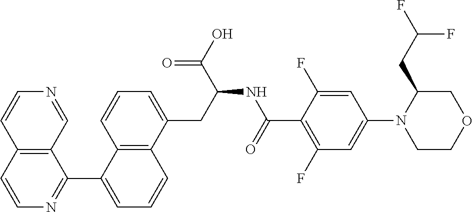 [SMILES: O=C(N[C@@H](Cc1cccc2c(-c3nccc4ccncc34)cccc12)C(=O)O)c1c(F)cc(N2CCOC[C@@H]2CC(F)F)cc1F] |
| 40 | 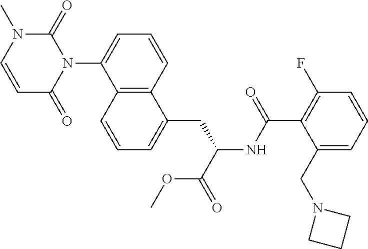 [SMILES: COC(=O)[C@H](Cc1cccc2c(-n3c(=O)ccn(C)c3=O)cccc12)NC(=O)c1c(F)cccc1CN1CCC1] |
| 41 | 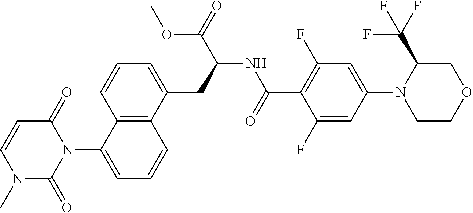 [SMILES: COC(=O)[C@H](Cc1cccc2c(-n3c(=O)ccn(C)c3=O)cccc12)NC(=O)c1c(F)cc(N2CCOC[C@@H]2C(F)(F)F)cc1F] |
| 42 | 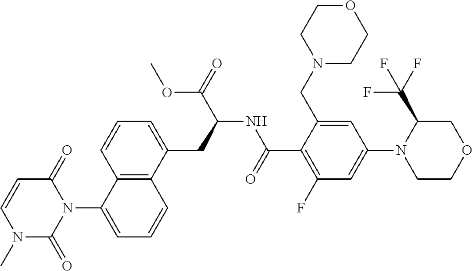 [SMILES: COC(=O)[C@H](Cc1cccc2c(-n3c(=O)ccn(C)c3=O)cccc12)NC(=O)c1c(F)cc(N2CCOC[C@@H]2C(F)(F)F)cc1CN1CCOCC1] |
| 43 | 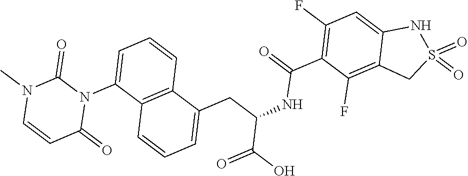 [SMILES: Cn1ccc(=O)n(-c2cccc3c(C[C@H](NC(=O)c4c(F)cc5c(c4F)CS(=O)(=O)N5)C(=O)O)cccc23)c1=O] |
| 44 | 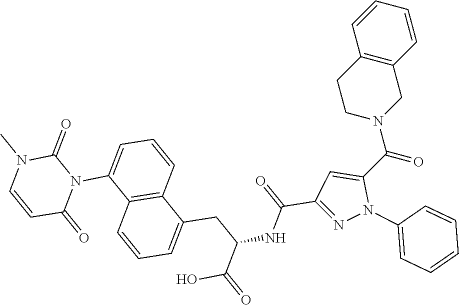 [SMILES: Cn1ccc(=O)n(-c2cccc3c(C[C@H](NC(=O)c4cc(C(=O)N5CCc6ccccc6C5)n(-c5ccccc5)n4)C(=O)O)cccc23)c1=O] |
| 45 | 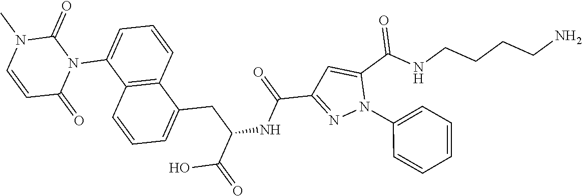 [SMILES: Cn1ccc(=O)n(-c2cccc3c(C[C@H](NC(=O)c4cc(C(=O)NCCCCN)n(-c5ccccc5)n4)C(=O)O)cccc23)c1=O] |
| 46 | 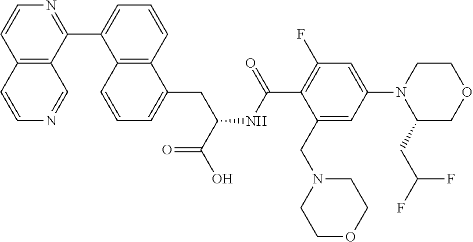 [SMILES: O=C(N[C@@H](Cc1cccc2c(-c3nccc4ccncc34)cccc12)C(=O)O)c1c(F)cc(N2CCOC[C@@H]2CC(F)F)cc1CN1CCOCC1] |
| 47 | 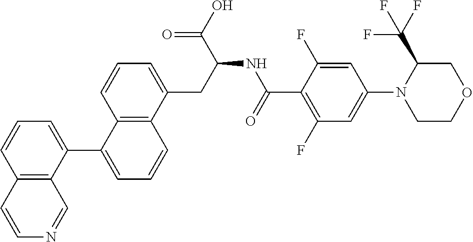 [SMILES: O=C(N[C@@H](Cc1cccc2c(-c3cccc4ccncc34)cccc12)C(=O)O)c1c(F)cc(N2CCOC[C@@H]2C(F)(F)F)cc1F] |
| 48 | 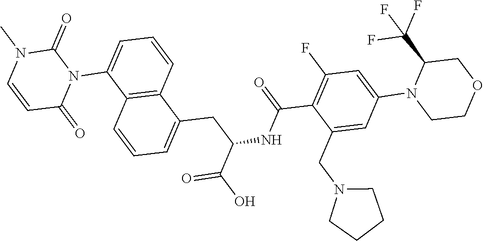 [SMILES: Cn1ccc(=O)n(-c2cccc3c(C[C@H](NC(=O)c4c(F)cc(N5CCOC[C@@H]5C(F)(F)F)cc4CN4CCCC4)C(=O)O)cccc23)c1=O] |
| 49 | 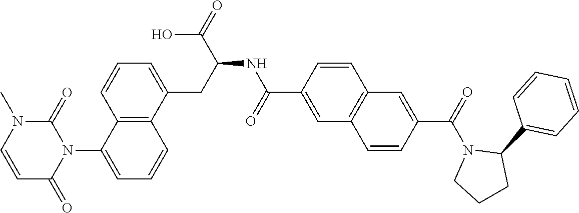 [SMILES: Cn1ccc(=O)n(-c2cccc3c(C[C@H](NC(=O)c4ccc5cc(C(=O)N6CCC[C@@H]6c6ccccc6)ccc5c4)C(=O)O)cccc23)c1=O] |
| 50 | 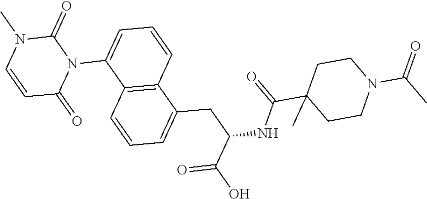 [SMILES: CC(=O)N1CCC(C)(C(=O)N[C@@H](Cc2cccc3c(-n4c(=O)ccn(C)c4=O)cccc23)C(=O)O)CC1] |
| 51 | 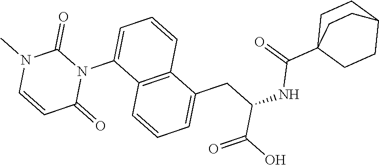 [SMILES: Cn1ccc(=O)n(-c2cccc3c(C[C@H](NC(=O)C45CCC(CC4)CC5)C(=O)O)cccc23)c1=O] |
| 52 | 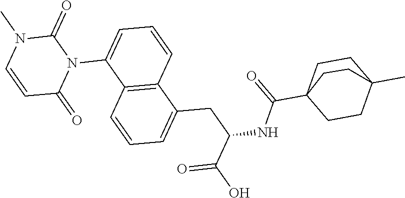 |
| 53 | 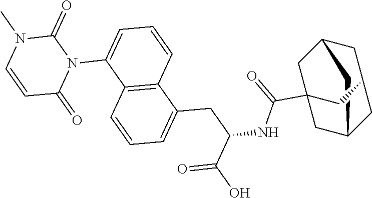 [SMILES: Cn1ccc(=O)n(-c2cccc3c(C[C@H](NC(=O)C45C[C@H]6C[C@@H](C4)C[C@@H](C5)C6)C(=O)O)cccc23)c1=O] |
| 54 | 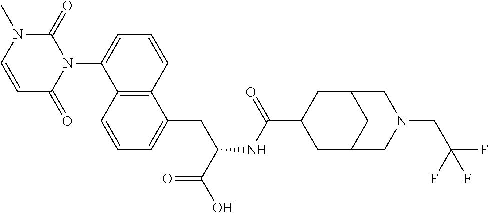 [SMILES: Cn1ccc(=O)n(-c2cccc3c(C[C@H](NC(=O)C4CC5CC(C4)CN(CC(F)(F)F)C5)C(=O)O)cccc23)c1=O] |
| 55 | 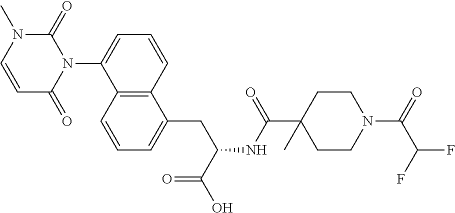 [SMILES: Cn1ccc(=O)n(-c2cccc3c(C[C@H](NC(=O)C4(C)CCN(C(=O)C(F)F)CC4)C(=O)O)cccc23)c1=O] |
| 56 | 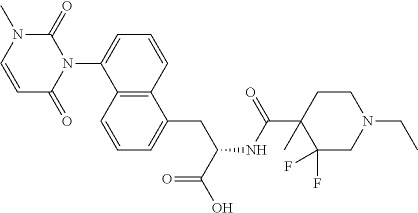 [SMILES: CCN1CCC(C)(C(=O)N[C@@H](Cc2cccc3c(-n4c(=O)ccn(C)c4=O)cccc23)C(=O)O)C(F)(F)C1] |
| 57 | 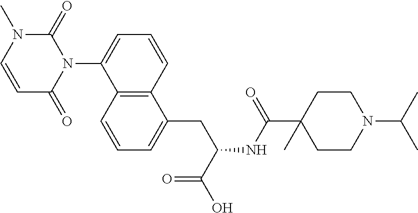 [SMILES: CC(C)N1CCC(C)(C(=O)N[C@@H](Cc2cccc3c(-n4c(=O)ccn(C)c4=O)cccc23)C(=O)O)CC1] |
| 58 | 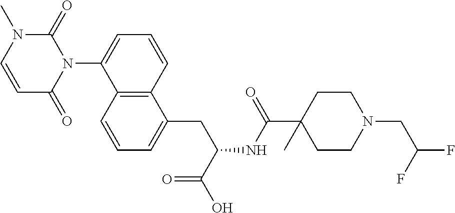 [SMILES: Cn1ccc(=O)n(-c2cccc3c(C[C@H](NC(=O)C4(C)CCN(CC(F)F)CC4)C(=O)O)cccc23)c1=O] |
| 59 | 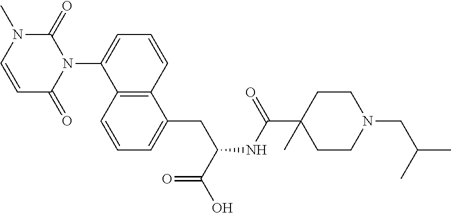 [SMILES: CC(C)CN1CCC(C)(C(=O)N[C@@H](Cc2cccc3c(-n4c(=O)ccn(C)c4=O)cccc23)C(=O)O)CC1] |
| 60 | 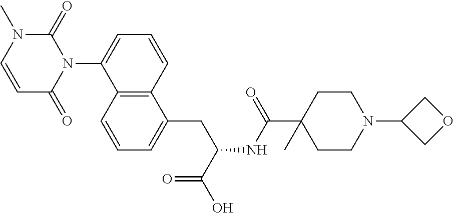 [SMILES: Cn1ccc(=O)n(-c2cccc3c(C[C@H](NC(=O)C4(C)CCN(C5COC5)CC4)C(=O)O)cccc23)c1=O] |
| 61 | 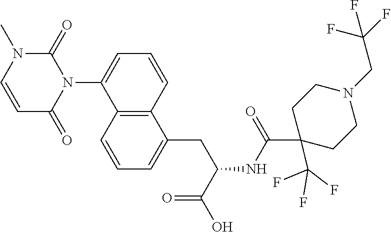 [SMILES: Cn1ccc(=O)n(-c2cccc3c(C[C@H](NC(=O)C4(C(F)(F)F)CCN(CC(F)(F)F)CC4)C(=O)O)cccc23)c1=O] |
| 62 | 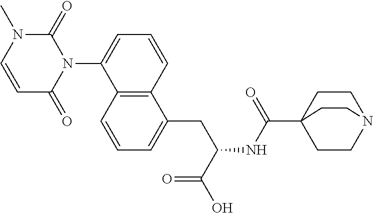 [SMILES: Cn1ccc(=O)n(-c2cccc3c(C[C@H](NC(=O)C45CCN(CC4)CC5)C(=O)O)cccc23)c1=O] |
| 63 | 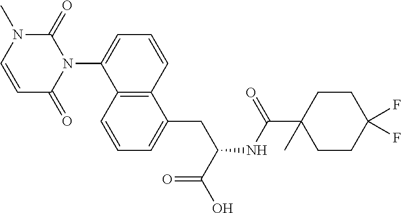 [SMILES: Cn1ccc(=O)n(-c2cccc3c(C[C@H](NC(=O)C4(C)CCC(F)(F)CC4)C(=O)O)cccc23)c1=O] |
| 64 | 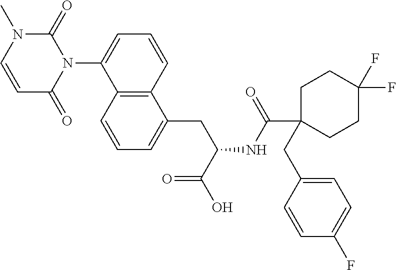 [SMILES: Cn1ccc(=O)n(-c2cccc3c(C[C@H](NC(=O)C4(Cc5ccc(F)cc5)CCC(F)(F)CC4)C(=O)O)cccc23)c1=O] |
| 65 | 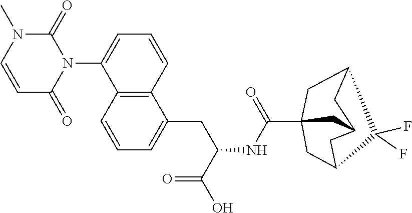 [SMILES: Cn1ccc(=O)n(-c2cccc3c(C[C@H](NC(=O)[C@]45C[C@@H]6C[C@H](C4)C(F)(F)[C@@H](C6)C5)C(=O)O)cccc23)c1=O] |
| 66 | 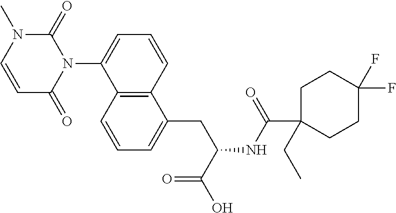 [SMILES: CCC1(C(=O)N[C@@H](Cc2cccc3c(-n4c(=O)ccn(C)c4=O)cccc23)C(=O)O)CCC(F)(F)CC1] |
| 67 | 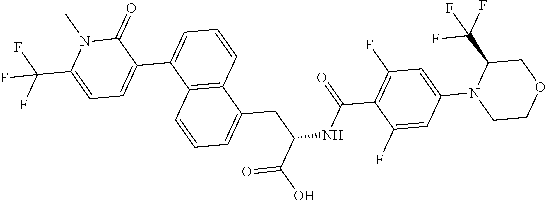 [SMILES: Cn1c(C(F)(F)F)ccc(-c2cccc3c(C[C@H](NC(=O)c4c(F)cc(N5CCOC[C@@H]5C(F)(F)F)cc4F)C(=O)O)cccc23)c1=O] |
| 68 | 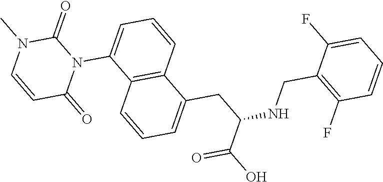 [SMILES: Cn1ccc(=O)n(-c2cccc3c(C[C@H](NCc4c(F)cccc4F)C(=O)O)cccc23)c1=O] |
| 69 | 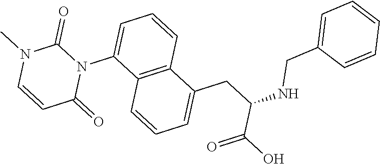 [SMILES: Cn1ccc(=O)n(-c2cccc3c(C[C@H](NCc4ccccc4)C(=O)O)cccc23)c1=O] |
| 70 | 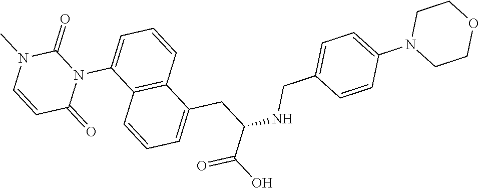 [SMILES: Cn1ccc(=O)n(-c2cccc3c(C[C@H](NCc4ccc(N5CCOCC5)cc4)C(=O)O)cccc23)c1=O] |
| 71 | 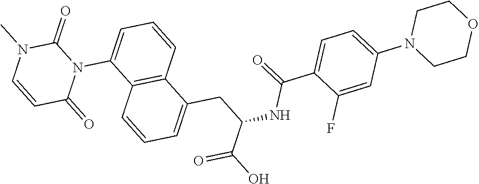 [SMILES: Cn1ccc(=O)n(-c2cccc3c(C[C@H](NC(=O)c4ccc(N5CCOCC5)cc4F)C(=O)O)cccc23)c1=O] |
| 72 | 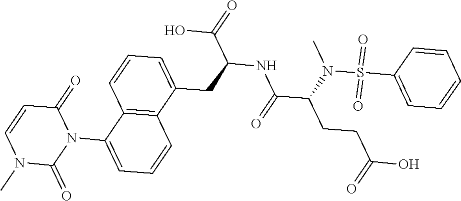 [SMILES: CN([C@H](CCC(=O)O)C(=O)N[C@@H](Cc1cccc2c(-n3c(=O)ccn(C)c3=O)cccc12)C(=O)O)S(=O)(=O)c1ccccc1] |
| 73 | 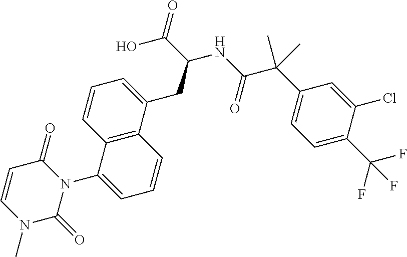 [SMILES: Cn1ccc(=O)n(-c2cccc3c(C[C@H](NC(=O)C(C)(C)c4ccc(C(F)(F)F)c(Cl)c4)C(=O)O)cccc23)c1=O] |
| 74 | 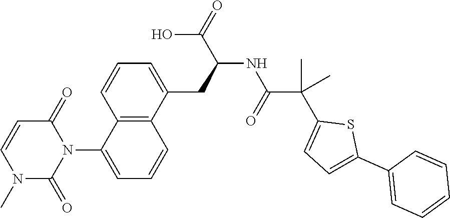 [SMILES: Cn1ccc(=O)n(-c2cccc3c(C[C@H](NC(=O)C(C)(C)c4ccc(-c5ccccc5)s4)C(=O)O)cccc23)c1=O] |
| 75 | 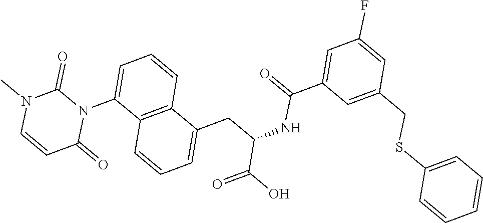 [SMILES: Cn1ccc(=O)n(-c2cccc3c(C[C@H](NC(=O)c4cc(F)cc(CSc5ccccc5)c4)C(=O)O)cccc23)c1=O] |
| 76 | 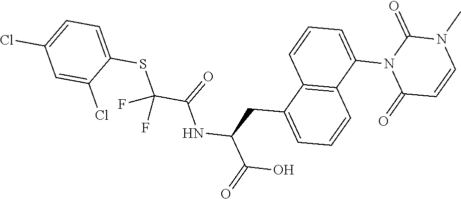 [SMILES: Cn1ccc(=O)n(-c2cccc3c(C[C@H](NC(=O)C(F)(F)Sc4ccc(Cl)cc4Cl)C(=O)O)cccc23)c1=O] |
| 77 | 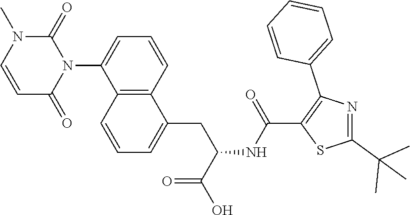 [SMILES: Cn1ccc(=O)n(-c2cccc3c(C[C@H](NC(=O)c4sc(C(C)(C)C)nc4-c4ccccc4)C(=O)O)cccc23)c1=O] |
| 78 | 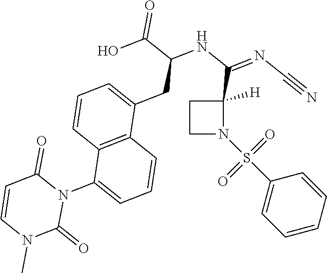 [SMILES: Cn1ccc(=O)n(-c2cccc3c(C[C@H](N/C(=N/C#N)[C@@H]4CCN4S(=O)(=O)c4ccccc4)C(=O)O)cccc23)c1=O] |
| 79 | 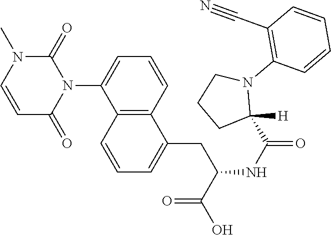 [SMILES: Cn1ccc(=O)n(-c2cccc3c(C[C@H](NC(=O)[C@@H]4CCCN4c4ccccc4C#N)C(=O)O)cccc23)c1=O] |
| 80 | 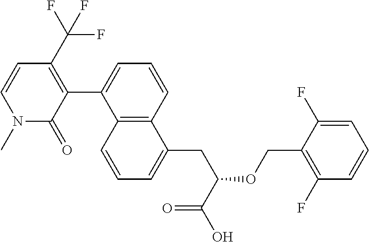 [SMILES: Cn1ccc(C(F)(F)F)c(-c2cccc3c(C[C@H](OCc4c(F)cccc4F)C(=O)O)cccc23)c1=O] |
| 81 | 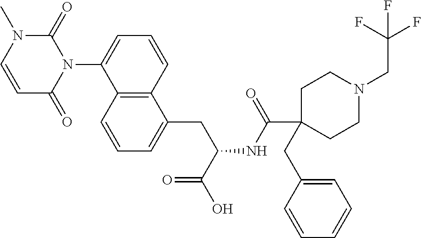 [SMILES: Cn1ccc(=O)n(-c2cccc3c(C[C@H](NC(=O)C4(Cc5ccccc5)CCN(CC(F)(F)F)CC4)C(=O)O)cccc23)c1=O] |
| 82 | 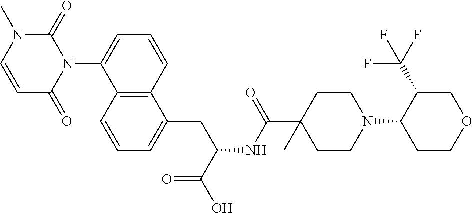 [SMILES: Cn1ccc(=O)n(-c2cccc3c(C[C@H](NC(=O)C4(C)CCN([C@H]5CCOC[C@H]5C(F)(F)F)CC4)C(=O)O)cccc23)c1=O] |
| 83 | 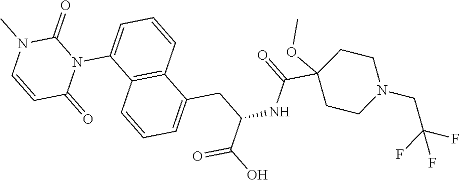 [SMILES: COC1(C(=O)N[C@@H](Cc2cccc3c(-n4c(=O)ccn(C)c4=O)cccc23)C(=O)O)CCN(CC(F)(F)F)CC1] |
| 84 | 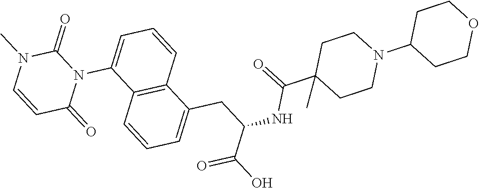 [SMILES: Cn1ccc(=O)n(-c2cccc3c(C[C@H](NC(=O)C4(C)CCN(C5CCOCC5)CC4)C(=O)O)cccc23)c1=O] |
| 85 | 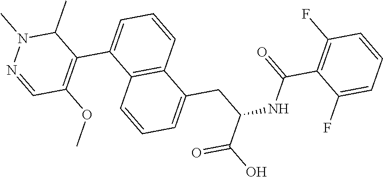 [SMILES: COC1=C(c2cccc3c(C[C@H](NC(=O)c4c(F)cccc4F)C(=O)O)cccc23)C(C)N(C)N=C1] |
| 86 | 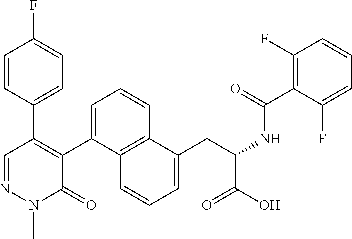 [SMILES: Cn1ncc(-c2ccc(F)cc2)c(-c2cccc3c(C[C@H](NC(=O)c4c(F)cccc4F)C(=O)O)cccc23)c1=O] |
| 87 | 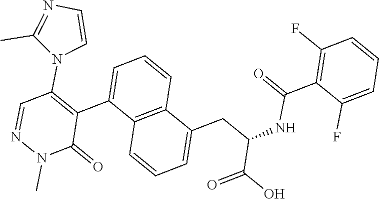 [SMILES: Cc1nccn1-c1cnn(C)c(=O)c1-c1cccc2c(C[C@H](NC(=O)c3c(F)cccc3F)C(=O)O)cccc12] |
| 88 | 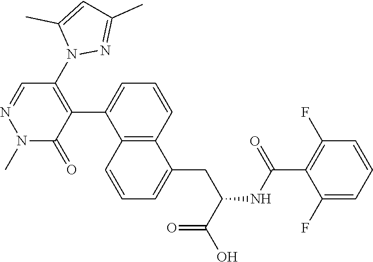 [SMILES: Cc1cc(C)n(-c2cnn(C)c(=O)c2-c2cccc3c(C[C@H](NC(=O)c4c(F)cccc4F)C(=O)O)cccc23)n1] |
| 89 | 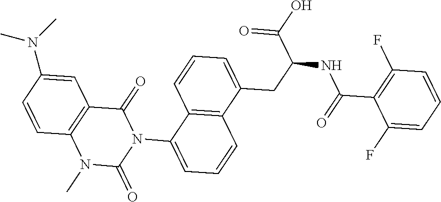 [SMILES: CN(C)c1ccc2c(c1)c(=O)n(-c1cccc3c(C[C@H](NC(=O)c4c(F)cccc4F)C(=O)O)cccc13)c(=O)n2C] |
| 90 | 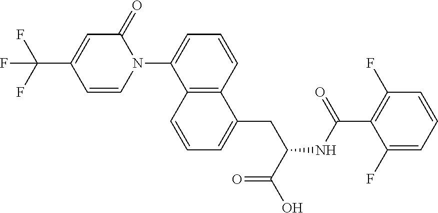 [SMILES: O=C(N[C@@H](Cc1cccc2c(-n3ccc(C(F)(F)F)cc3=O)cccc12)C(=O)O)c1c(F)cccc1F] |
| 91 | 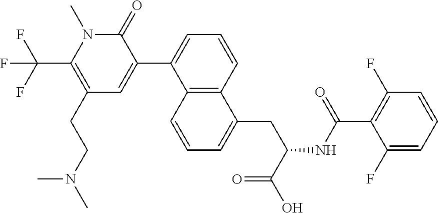 [SMILES: CN(C)CCc1cc(-c2cccc3c(C[C@H](NC(=O)c4c(F)cccc4F)C(=O)O)cccc23)c(=O)n(C)c1C(F)(F)F] |
| 92 | 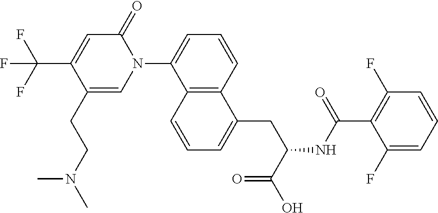 [SMILES: CN(C)CCc1cn(-c2cccc3c(C[C@H](NC(=O)c4c(F)cccc4F)C(=O)O)cccc23)c(=O)cc1C(F)(F)F] |
| 93 | 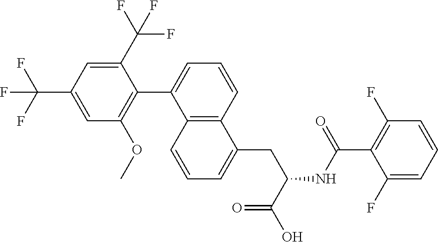 [SMILES: COc1cc(C(F)(F)F)cc(C(F)(F)F)c1-c1cccc2c(C[C@H](NC(=O)c3c(F)cccc3F)C(=O)O)cccc12] |
| 94 | 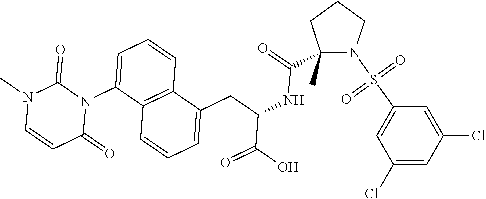 [SMILES: Cn1ccc(=O)n(-c2cccc3c(C[C@H](NC(=O)[C@]4(C)CCCN4S(=O)(=O)c4cc(Cl)cc(Cl)c4)C(=O)O)cccc23)c1=O] |
| 95 | 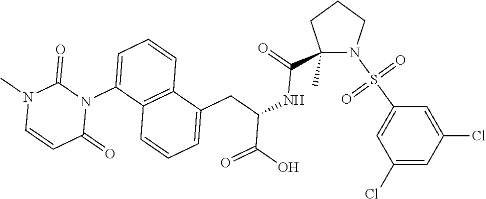 [SMILES: Cn1ccc(=O)n(-c2cccc3c(C[C@H](NC(=O)[C@@]4(C)CCCN4S(=O)(=O)c4cc(Cl)cc(Cl)c4)C(=O)O)cccc23)c1=O] |
| 96 | 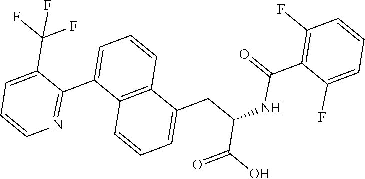 [SMILES: O=C(N[C@@H](Cc1cccc2c(-c3ncccc3C(F)(F)F)cccc12)C(=O)O)c1c(F)cccc1F] |
| 97 | 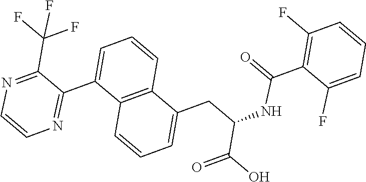 [SMILES: O=C(N[C@@H](Cc1cccc2c(-c3nccnc3C(F)(F)F)cccc12)C(=O)O)c1c(F)cccc1F] |
| 98 | 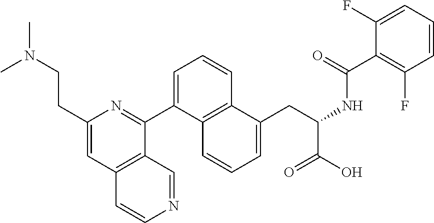 [SMILES: CN(C)CCc1cc2ccncc2c(-c2cccc3c(C[C@H](NC(=O)c4c(F)cccc4F)C(=O)O)cccc23)n1] |
| 99 | 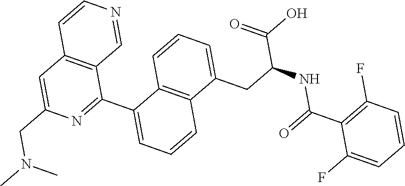 [SMILES: CN(C)Cc1cc2ccncc2c(-c2cccc3c(C[C@H](NC(=O)c4c(F)cccc4F)C(=O)O)cccc23)n1] |
| 100 | 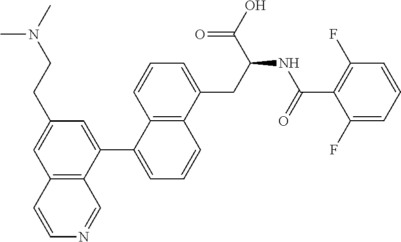 [SMILES: CN(C)CCc1cc(-c2cccc3c(C[C@H](NC(=O)c4c(F)cccc4F)C(=O)O)cccc23)c2cnccc2c1] |
| 101 | 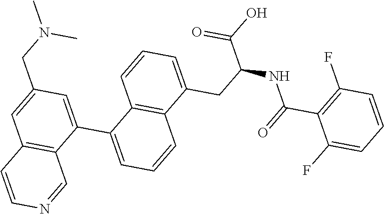 [SMILES: CN(C)Cc1cc(-c2cccc3c(C[C@H](NC(=O)c4c(F)cccc4F)C(=O)O)cccc23)c2cnccc2c1] |
| 102 | 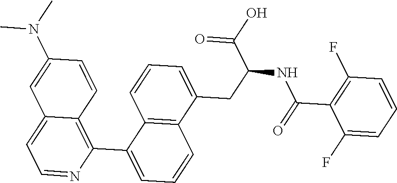 [SMILES: CN(C)c1ccc2c(-c3cccc4c(C[C@H](NC(=O)c5c(F)cccc5F)C(=O)O)cccc34)nccc2c1] |
| 103 | 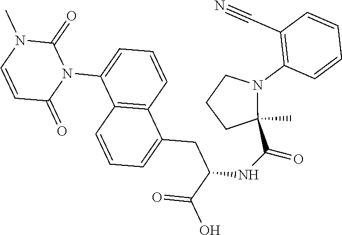 [SMILES: Cn1ccc(=O)n(-c2cccc3c(C[C@H](NC(=O)[C@@]4(C)CCCN4c4ccccc4C#N)C(=O)O)cccc23)c1=O] |
| 104 | 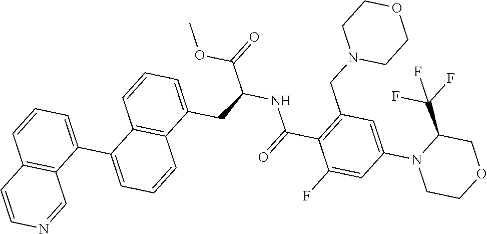 [SMILES: COC(=O)[C@H](Cc1cccc2c(-c3cccc4ccncc34)cccc12)NC(=O)c1c(F)cc(N2CCOC[C@@H]2C(F)(F)F)cc1CN1CCOCC1] |
| 105 | 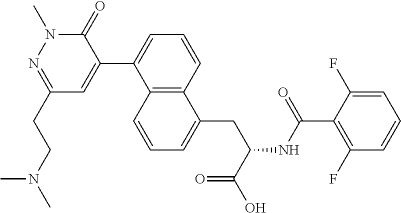 [SMILES: CN(C)CCc1cc(-c2cccc3c(C[C@H](NC(=O)c4c(F)cccc4F)C(=O)O)cccc23)c(=O)n(C)n1] |
| 106 | 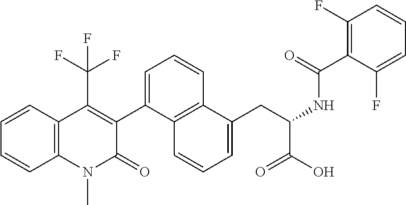 [SMILES: Cn1c(=O)c(-c2cccc3c(C[C@H](NC(=O)c4c(F)cccc4F)C(=O)O)cccc23)c(C(F)(F)F)c2ccccc21] |
| 107 | 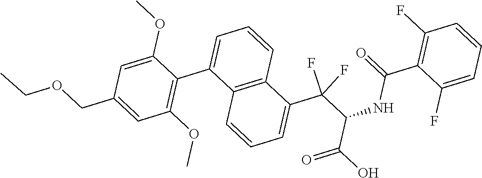 [SMILES: CCOCc1cc(OC)c(-c2cccc3c(C(F)(F)[C@H](NC(=O)c4c(F)cccc4F)C(=O)O)cccc23)c(OC)c1] |
| 108 | 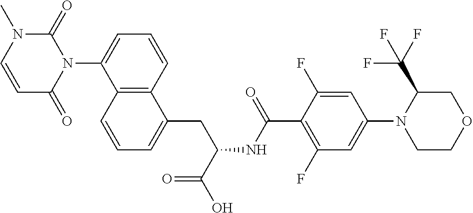 [SMILES: Cn1ccc(=O)n(-c2cccc3c(C[C@H](NC(=O)c4c(F)cc(N5CCOC[C@@H]5C(F)(F)F)cc4F)C(=O)O)cccc23)c1=O] |
| 109 | 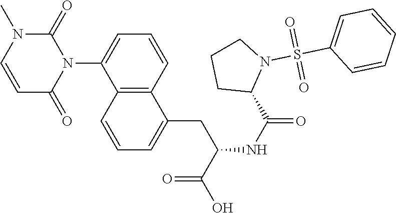 [SMILES: Cn1ccc(=O)n(-c2cccc3c(C[C@H](NC(=O)[C@@H]4CCCN4S(=O)(=O)c4ccccc4)C(=O)O)cccc23)c1=O] |
| 110 | 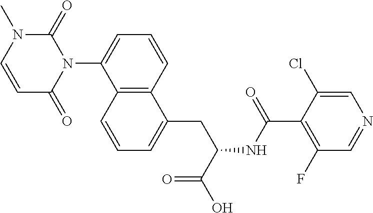 [SMILES: Cn1ccc(=O)n(-c2cccc3c(C[C@H](NC(=O)c4c(F)cncc4Cl)C(=O)O)cccc23)c1=O] |
| 111 | 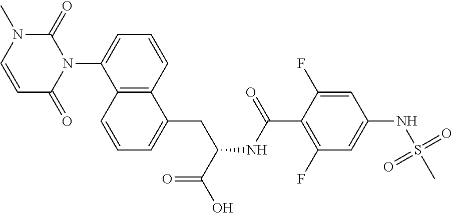 [SMILES: Cn1ccc(=O)n(-c2cccc3c(C[C@H](NC(=O)c4c(F)cc(NS(C)(=O)=O)cc4F)C(=O)O)cccc23)c1=O] |
| 112 | 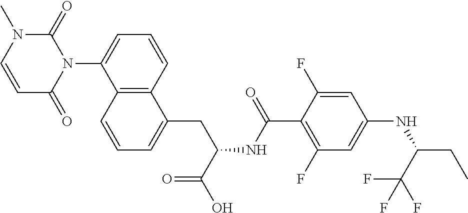 [SMILES: CC[C@@H](Nc1cc(F)c(C(=O)N[C@@H](Cc2cccc3c(-n4c(=O)ccn(C)c4=O)cccc23)C(=O)O)c(F)c1)C(F)(F)F] |
| 113 | 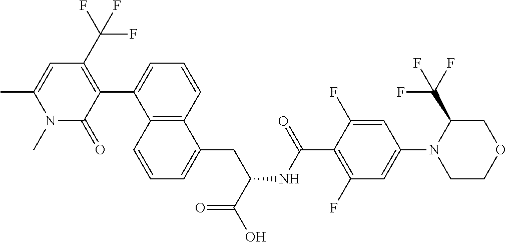 [SMILES: Cc1cc(C(F)(F)F)c(-c2cccc3c(C[C@H](NC(=O)c4c(F)cc(N5CCOC[C@@H]5C(F)(F)F)cc4F)C(=O)O)cccc23)c(=O)n1C] |
| 114 | 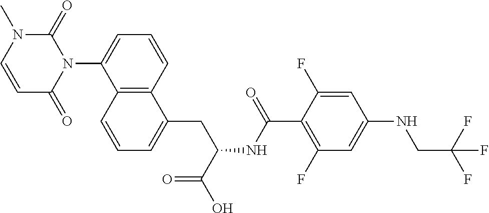 [SMILES: Cn1ccc(=O)n(-c2cccc3c(C[C@H](NC(=O)c4c(F)cc(NCC(F)(F)F)cc4F)C(=O)O)cccc23)c1=O] |
| 115 | 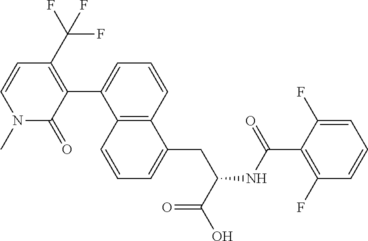 [SMILES: Cn1ccc(C(F)(F)F)c(-c2cccc3c(C[C@H](NC(=O)c4c(F)cccc4F)C(=O)O)cccc23)c1=O] |
| 116 | 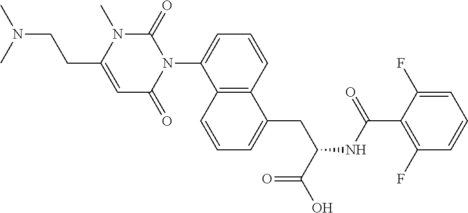 [SMILES: CN(C)CCc1cc(=O)n(-c2cccc3c(C[C@H](NC(=O)c4c(F)cccc4F)C(=O)O)cccc23)c(=O)n1C] |
| 117 | 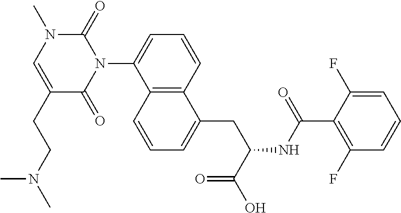 [SMILES: CN(C)CCc1cn(C)c(=O)n(-c2cccc3c(C[C@H](NC(=O)c4c(F)cccc4F)C(=O)O)cccc23)c1=O] |
| 118 | 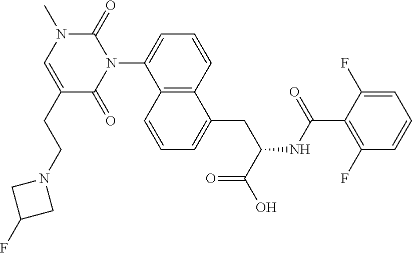 [SMILES: Cn1cc(CCN2CC(F)C2)c(=O)n(-c2cccc3c(C[C@H](NC(=O)c4c(F)cccc4F)C(=O)O)cccc23)c1=O] |
| 119 | 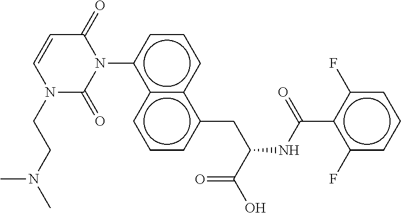 |
| 120 | 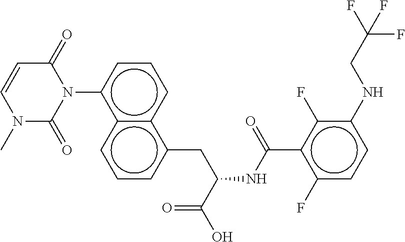 |
| 121 | 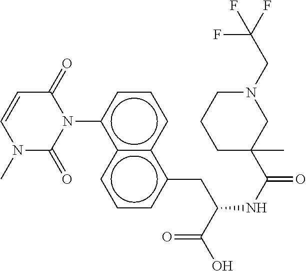 |
| 122 | 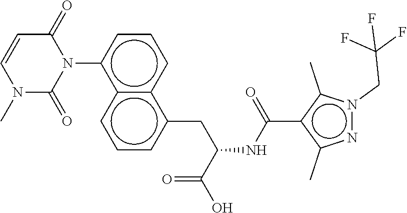 |
| 123 | 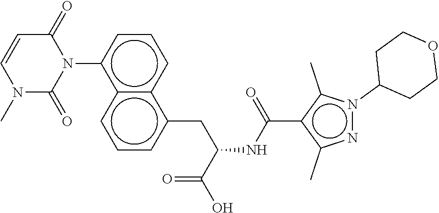 |
| 124 | 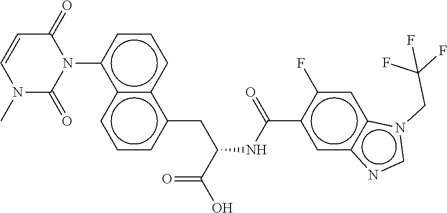 |
| 125 | 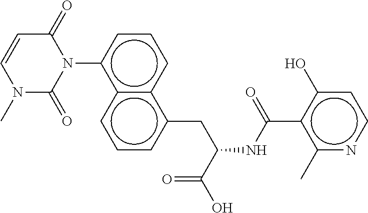 |
| 126 | 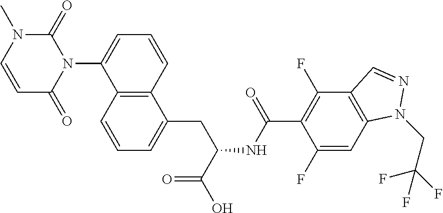 [SMILES: Cn1ccc(=O)n(-c2cccc3c(C[C@H](NC(=O)c4c(F)cc5c(cnn5CC(F)(F)F)c4F)C(=O)O)cccc23)c1=O] |
| 127 | 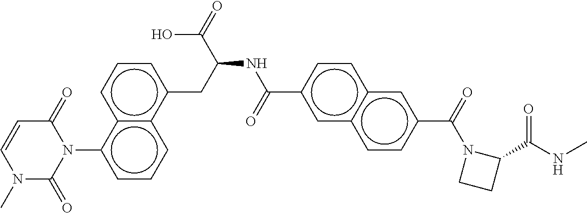 |
| 128 | 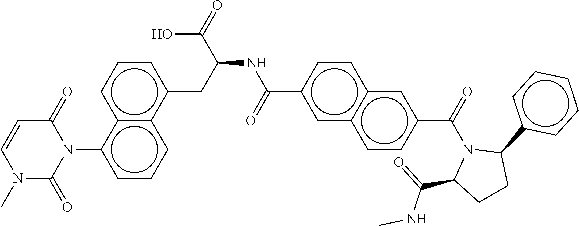 |
| 129 | 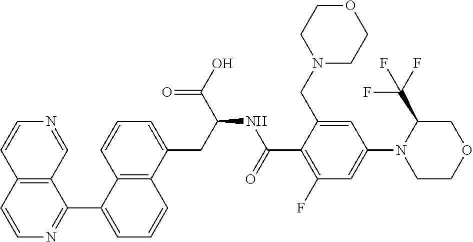 [SMILES: O=C(N[C@@H](Cc1cccc2c(-c3nccc4ccncc34)cccc12)C(=O)O)c1c(F)cc(N2CCOC[C@@H]2C(F)(F)F)cc1CN1CCOCC1] |
| 130 | 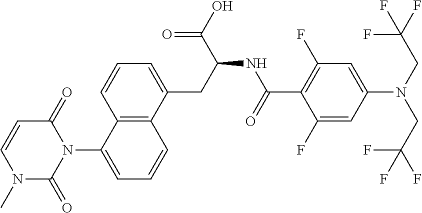 [SMILES: Cn1ccc(=O)n(-c2cccc3c(C[C@H](NC(=O)c4c(F)cc(N(CC(F)(F)F)CC(F)(F)F)cc4F)C(=O)O)cccc23)c1=O] |
| 131 | 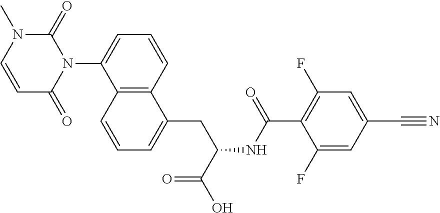 [SMILES: Cn1ccc(=O)n(-c2cccc3c(C[C@H](NC(=O)c4c(F)cc(C#N)cc4F)C(=O)O)cccc23)c1=O] |
| 132 | 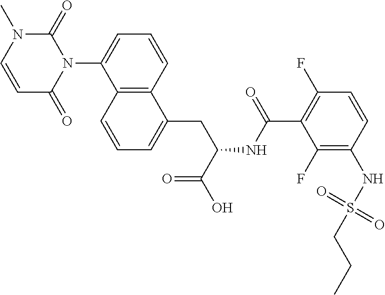 [SMILES: CCCS(=O)(=O)Nc1ccc(F)c(C(=O)N[C@@H](Cc2cccc3c(-n4c(=O)ccn(C)c4=O)cccc23)C(=O)O)c1F] |
| 133 | 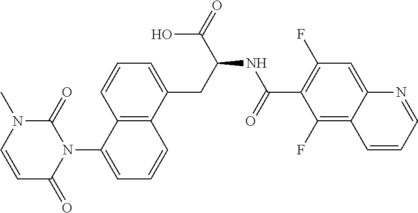 [SMILES: Cn1ccc(=O)n(-c2cccc3c(C[C@H](NC(=O)c4c(F)cc5ncccc5c4F)C(=O)O)cccc23)c1=O] |
| 134 | 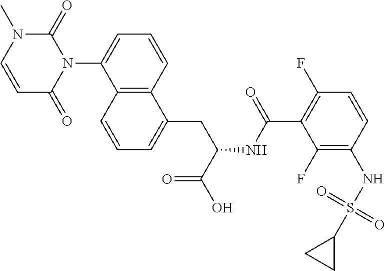 [SMILES: Cn1ccc(=O)n(-c2cccc3c(C[C@H](NC(=O)c4c(F)ccc(NS(=O)(=O)C5CC5)c4F)C(=O)O)cccc23)c1=O] |
| 135 | 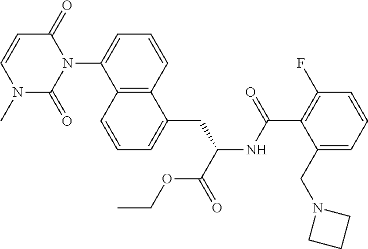 [SMILES: CCOC(=O)[C@H](Cc1cccc2c(-n3c(=O)ccn(C)c3=O)cccc12)NC(=O)c1c(F)cccc1CN1CCC1] |
| 136 | 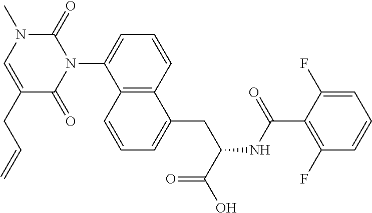 [SMILES: C=CCc1cn(C)c(=O)n(-c2cccc3c(C[C@H](NC(=O)c4c(F)cccc4F)C(=O)O)cccc23)c1=O] |
| 137 | 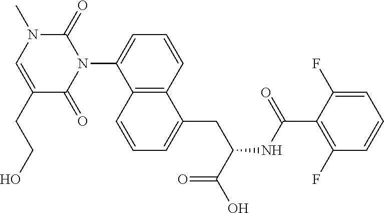 [SMILES: Cn1cc(CCO)c(=O)n(-c2cccc3c(C[C@H](NC(=O)c4c(F)cccc4F)C(=O)O)cccc23)c1=O] |
| 138 | 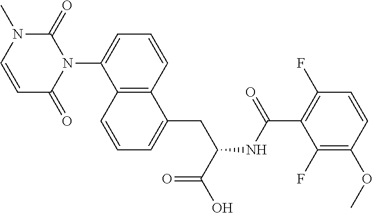 [SMILES: COc1ccc(F)c(C(=O)N[C@@H](Cc2cccc3c(-n4c(=O)ccn(C)c4=O)cccc23)C(=O)O)c1F] |
| 139 | 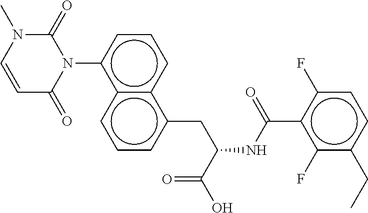 |
| 140 | 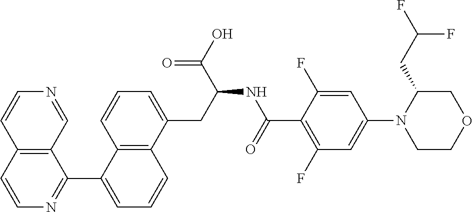 [SMILES: O=C(N[C@@H](Cc1cccc2c(-c3nccc4ccncc34)cccc12)C(=O)O)c1c(F)cc(N2CCOC[C@H]2CC(F)F)cc1F] |
| 141 | 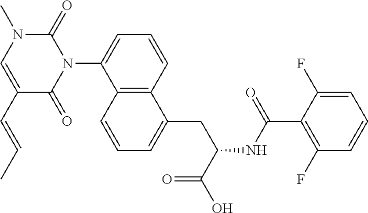 [SMILES: C/C=C/c1cn(C)c(=O)n(-c2cccc3c(C[C@H](NC(=O)c4c(F)cccc4F)C(=O)O)cccc23)c1=O] |
| 142 | 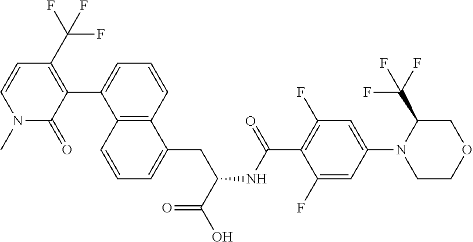 [SMILES: Cn1ccc(C(F)(F)F)c(-c2cccc3c(C[C@H](NC(=O)c4c(F)cc(N5CCOC[C@@H]5C(F)(F)F)cc4F)C(=O)O)cccc23)c1=O] |
| 143 | 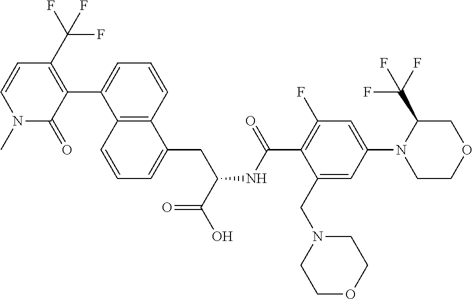 [SMILES: Cn1ccc(C(F)(F)F)c(-c2cccc3c(C[C@H](NC(=O)c4c(F)cc(N5CCOC[C@@H]5C(F)(F)F)cc4CN4CCOCC4)C(=O)O)cccc23)c1=O] |
| 144 | 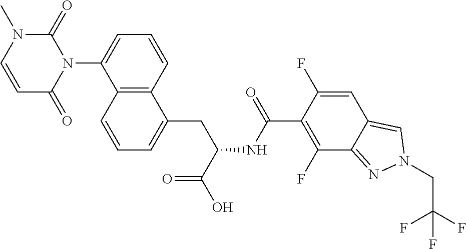 [SMILES: Cn1ccc(=O)n(-c2cccc3c(C[C@H](NC(=O)c4c(F)cc5cn(CC(F)(F)F)nc5c4F)C(=O)O)cccc23)c1=O] |
| 145 | 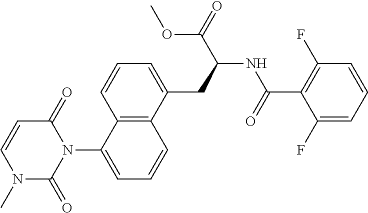 [SMILES: COC(=O)[C@H](Cc1cccc2c(-n3c(=O)ccn(C)c3=O)cccc12)NC(=O)c1c(F)cccc1F] |
| 146 | 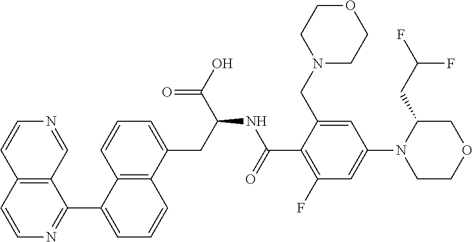 [SMILES: O=C(N[C@@H](Cc1cccc2c(-c3nccc4ccncc34)cccc12)C(=O)O)c1c(F)cc(N2CCOC[C@H]2CC(F)F)cc1CN1CCOCC1] |
| 147 | 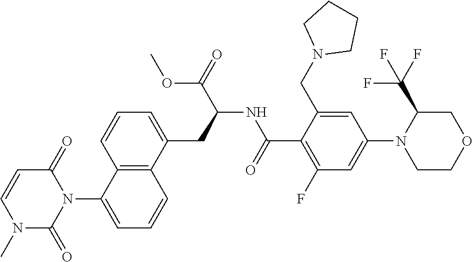 [SMILES: COC(=O)[C@H](Cc1cccc2c(-n3c(=O)ccn(C)c3=O)cccc12)NC(=O)c1c(F)cc(N2CCOC[C@@H]2C(F)(F)F)cc1CN1CCCC1] |
| 148 | 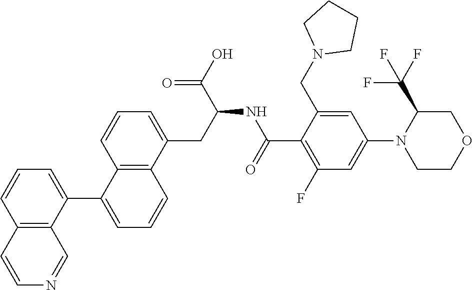 [SMILES: O=C(N[C@@H](Cc1cccc2c(-c3cccc4ccncc34)cccc12)C(=O)O)c1c(F)cc(N2CCOC[C@@H]2C(F)(F)F)cc1CN1CCCC1] |
| 149 | 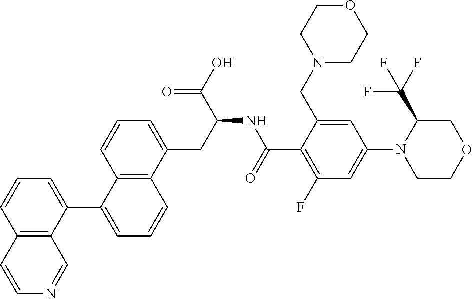 [SMILES: O=C(N[C@@H](Cc1cccc2c(-c3cccc4ccncc34)cccc12)C(=O)O)c1c(F)cc(N2CCOC[C@@H]2C(F)(F)F)cc1CN1CCOCC1] |
| 150 | 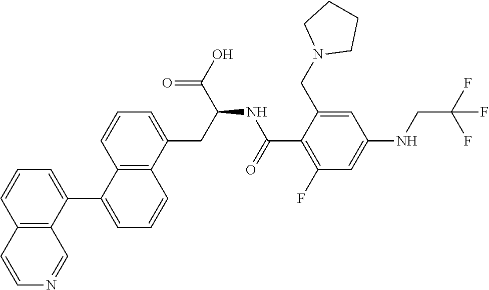 [SMILES: O=C(N[C@@H](Cc1cccc2c(-c3cccc4ccncc34)cccc12)C(=O)O)c1c(F)cc(NCC(F)(F)F)cc1CN1CCCC1] |
| 151 | 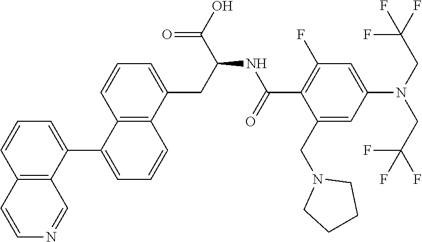 [SMILES: O=C(N[C@@H](Cc1cccc2c(-c3cccc4ccncc34)cccc12)C(=O)O)c1c(F)cc(N(CC(F)(F)F)CC(F)(F)F)cc1CN1CCCC1] |
| 152 | 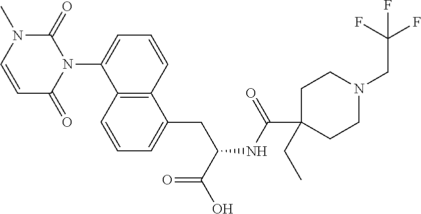 [SMILES: CCC1(C(=O)N[C@@H](Cc2cccc3c(-n4c(=O)ccn(C)c4=O)cccc23)C(=O)O)CCN(CC(F)(F)F)CC1] |
| 153 | 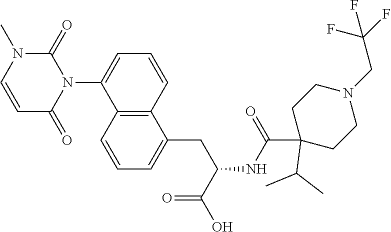 [SMILES: CC(C)C1(C(=O)N[C@@H](Cc2cccc3c(-n4c(=O)ccn(C)c4=O)cccc23)C(=O)O)CCN(CC(F)(F)F)CC1] |
| 154 | 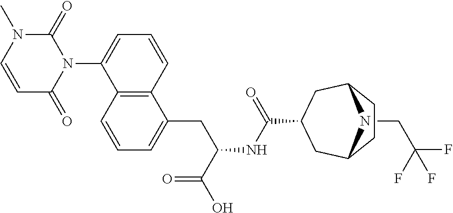 [SMILES: Cn1ccc(=O)n(-c2cccc3c(C[C@H](NC(=O)[C@H]4C[C@H]5CC[C@@H](C4)N5CC(F)(F)F)C(=O)O)cccc23)c1=O] |
| 155 | 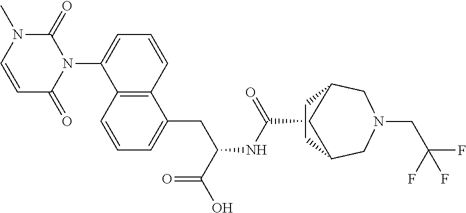 [SMILES: Cn1ccc(=O)n(-c2cccc3c(C[C@H](NC(=O)[C@@H]4[C@@H]5CC[C@H]4CN(CC(F)(F)F)C5)C(=O)O)cccc23)c1=O] |
| 156 | 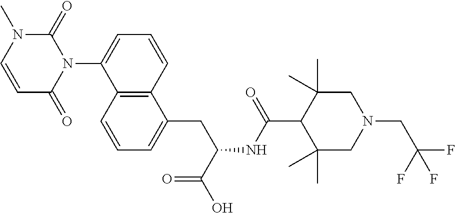 [SMILES: Cn1ccc(=O)n(-c2cccc3c(C[C@H](NC(=O)C4C(C)(C)CN(CC(F)(F)F)CC4(C)C)C(=O)O)cccc23)c1=O] |
| 157 | 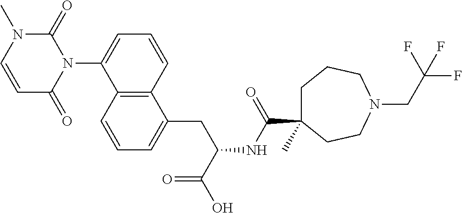 [SMILES: Cn1ccc(=O)n(-c2cccc3c(C[C@H](NC(=O)[C@@]4(C)CCCN(CC(F)(F)F)CC4)C(=O)O)cccc23)c1=O] |
| 158 | 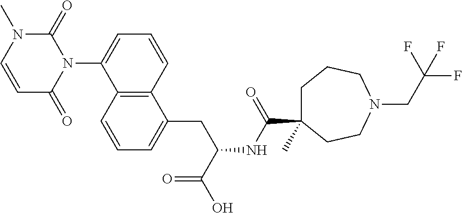 [SMILES: Cn1ccc(=O)n(-c2cccc3c(C[C@H](NC(=O)[C@@]4(C)CCCN(CC(F)(F)F)CC4)C(=O)O)cccc23)c1=O] |
| 159 | 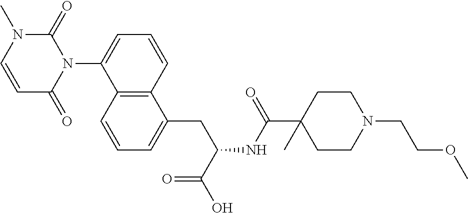 [SMILES: COCCN1CCC(C)(C(=O)N[C@@H](Cc2cccc3c(-n4c(=O)ccn(C)c4=O)cccc23)C(=O)O)CC1] |
| 160 | 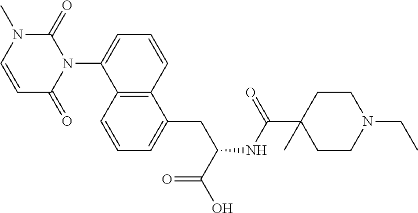 [SMILES: CCN1CCC(C)(C(=O)N[C@@H](Cc2cccc3c(-n4c(=O)ccn(C)c4=O)cccc23)C(=O)O)CC1] |
| 161 | 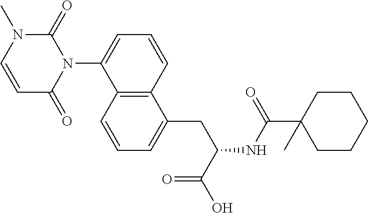 [SMILES: Cn1ccc(=O)n(-c2cccc3c(C[C@H](NC(=O)C4(C)CCCCC4)C(=O)O)cccc23)c1=O] |
| 162 | 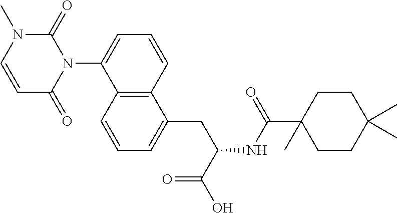 [SMILES: Cn1ccc(=O)n(-c2cccc3c(C[C@H](NC(=O)C4(C)CCC(C)(C)CC4)C(=O)O)cccc23)c1=O] |
| 163 | 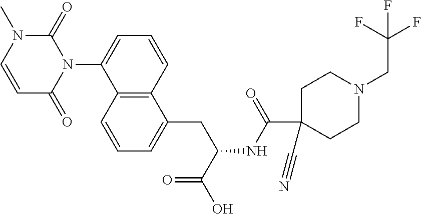 [SMILES: Cn1ccc(=O)n(-c2cccc3c(C[C@H](NC(=O)C4(C#N)CCN(CC(F)(F)F)CC4)C(=O)O)cccc23)c1=O] |
| 164 | 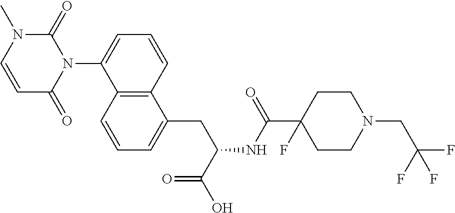 [SMILES: Cn1ccc(=O)n(-c2cccc3c(C[C@H](NC(=O)C4(F)CCN(CC(F)(F)F)CC4)C(=O)O)cccc23)c1=O] |
| 165 | 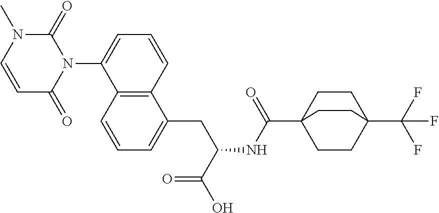 [SMILES: Cn1ccc(=O)n(-c2cccc3c(C[C@H](NC(=O)C45CCC(C(F)(F)F)(CC4)CC5)C(=O)O)cccc23)c1=O] |
| 166 | 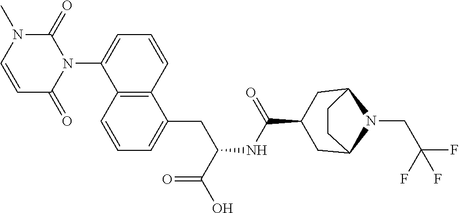 [SMILES: Cn1ccc(=O)n(-c2cccc3c(C[C@H](NC(=O)[C@@H]4C[C@H]5CC[C@@H](C4)N5CC(F)(F)F)C(=O)O)cccc23)c1=O] |
| 167 | 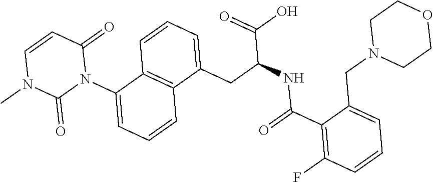 [SMILES: Cn1ccc(=O)n(-c2cccc3c(C[C@H](NC(=O)c4c(F)cccc4CN4CCOCC4)C(=O)O)cccc23)c1=O] |
| 168 | 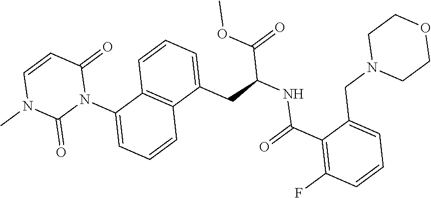 [SMILES: COC(=O)[C@H](Cc1cccc2c(-n3c(=O)ccn(C)c3=O)cccc12)NC(=O)c1c(F)cccc1CN1CCOCC1] |
| 169 | 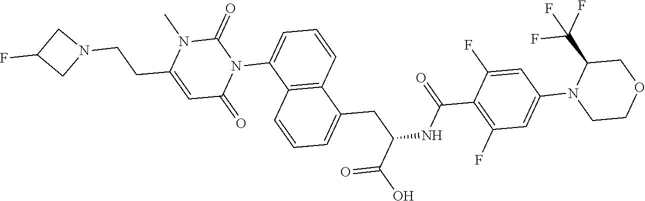 [SMILES: Cn1c(CCN2CC(F)C2)cc(=O)n(-c2cccc3c(C[C@H](NC(=O)c4c(F)cc(N5CCOC[C@@H]5C(F)(F)F)cc4F)C(=O)O)cccc23)c1=O] |
| 170 | 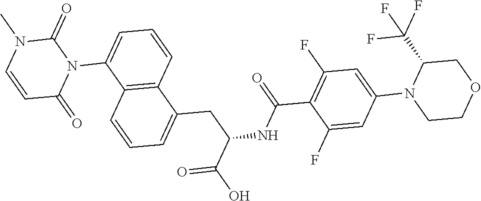 [SMILES: Cn1ccc(=O)n(-c2cccc3c(C[C@H](NC(=O)c4c(F)cc(N5CCOC[C@H]5C(F)(F)F)cc4F)C(=O)O)cccc23)c1=O] |
| 171 | 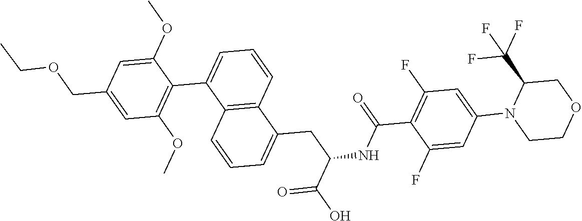 [SMILES: CCOCc1cc(OC)c(-c2cccc3c(C[C@H](NC(=O)c4c(F)cc(N5CCOC[C@@H]5C(F)(F)F)cc4F)C(=O)O)cccc23)c(OC)c1] |
| 172 | 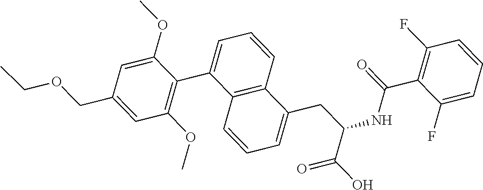 [SMILES: CCOCc1cc(OC)c(-c2cccc3c(C[C@H](NC(=O)c4c(F)cccc4F)C(=O)O)cccc23)c(OC)c1] |
| 173 | 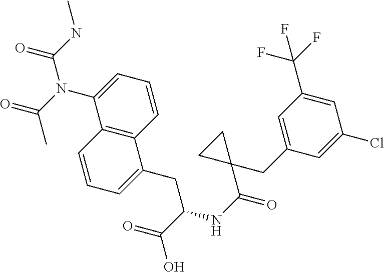 [SMILES: CNC(=O)N(C(C)=O)c1cccc2c(C[C@H](NC(=O)C3(Cc4cc(Cl)cc(C(F)(F)F)c4)CC3)C(=O)O)cccc12] |
| 174 | 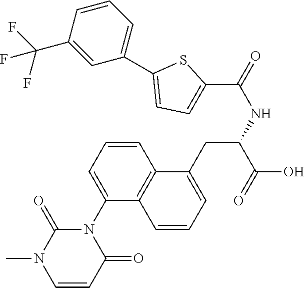 [SMILES: Cn1ccc(=O)n(-c2cccc3c(C[C@H](NC(=O)c4ccc(-c5cccc(C(F)(F)F)c5)s4)C(=O)O)cccc23)c1=O] |
| 175 | 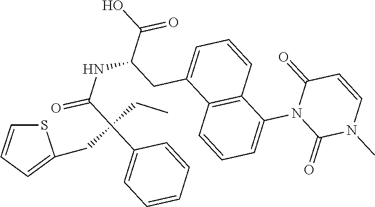 [SMILES: CC[C@](Cc1cccs1)(C(=O)N[C@@H](Cc1cccc2c(-n3c(=O)ccn(C)c3=O)cccc12)C(=O)O)c1ccccc1] |
| 176 | 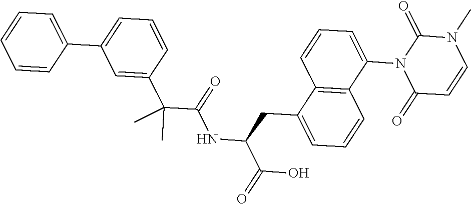 [SMILES: Cn1ccc(=O)n(-c2cccc3c(C[C@H](NC(=O)C(C)(C)c4cccc(-c5ccccc5)c4)C(=O)O)cccc23)c1=O] |
| 177 | 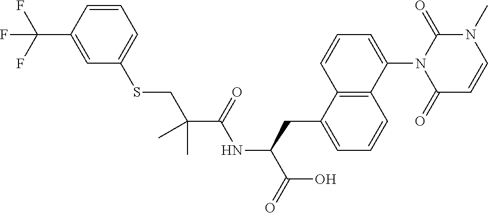 [SMILES: Cn1ccc(=O)n(-c2cccc3c(C[C@H](NC(=O)C(C)(C)CSc4cccc(C(F)(F)F)c4)C(=O)O)cccc23)c1=O] |
| 178 | 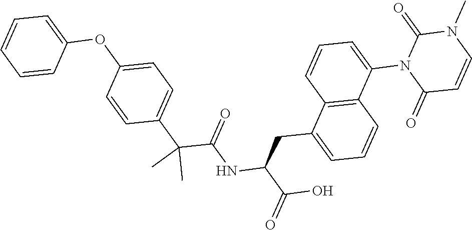 [SMILES: Cn1ccc(=O)n(-c2cccc3c(C[C@H](NC(=O)C(C)(C)c4ccc(Oc5ccccc5)cc4)C(=O)O)cccc23)c1=O] |
| 179 | 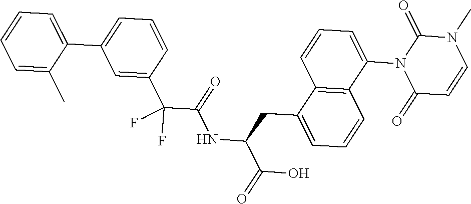 [SMILES: Cc1ccccc1-c1cccc(C(F)(F)C(=O)N[C@@H](Cc2cccc3c(-n4c(=O)ccn(C)c4=O)cccc23)C(=O)O)c1] |
| 180 | 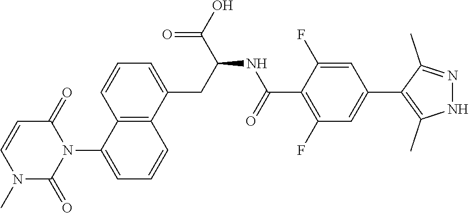 [SMILES: Cc1n[nH]c(C)c1-c1cc(F)c(C(=O)N[C@@H](Cc2cccc3c(-n4c(=O)ccn(C)c4=O)cccc23)C(=O)O)c(F)c1] |
| 181 | 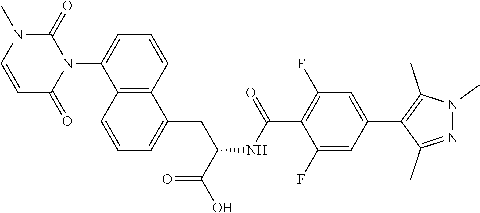 [SMILES: Cc1nn(C)c(C)c1-c1cc(F)c(C(=O)N[C@@H](Cc2cccc3c(-n4c(=O)ccn(C)c4=O)cccc23)C(=O)O)c(F)c1] |
| 182 | 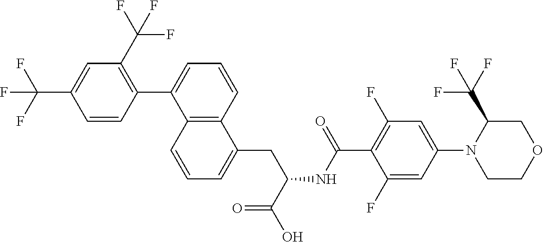 [SMILES: O=C(N[C@@H](Cc1cccc2c(-c3ccc(C(F)(F)F)cc3C(F)(F)F)cccc12)C(=O)O)c1c(F)cc(N2CCOC[C@@H]2C(F)(F)F)cc1F] |
| 183 | 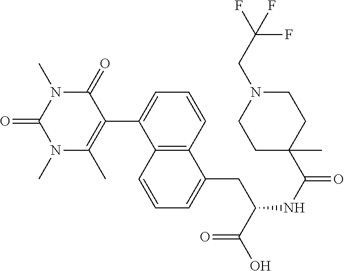 [SMILES: Cc1c(-c2cccc3c(C[C@H](NC(=O)C4(C)CCN(CC(F)(F)F)CC4)C(=O)O)cccc23)c(=O)n(C)c(=O)n1C] |
| 184 | 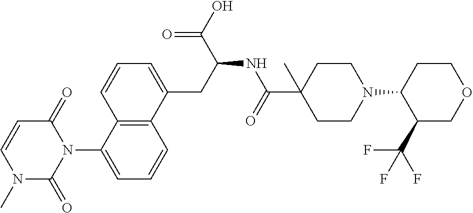 [SMILES: Cn1ccc(=O)n(-c2cccc3c(C[C@H](NC(=O)C4(C)CCN([C@@H]5CCOC[C@H]5C(F)(F)F)CC4)C(=O)O)cccc23)c1=O] |
| 185 | 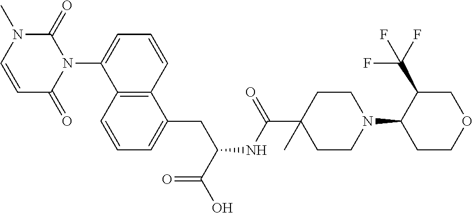 [SMILES: Cn1ccc(=O)n(-c2cccc3c(C[C@H](NC(=O)C4(C)CCN([C@@H]5CCOC[C@@H]5C(F)(F)F)CC4)C(=O)O)cccc23)c1=O] |
| 186 | 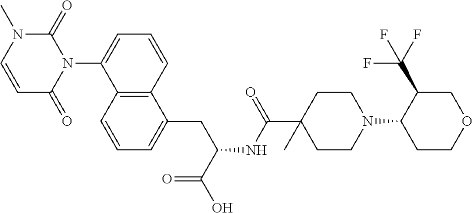 [SMILES: Cn1ccc(=O)n(-c2cccc3c(C[C@H](NC(=O)C4(C)CCN([C@H]5CCOC[C@@H]5C(F)(F)F)CC4)C(=O)O)cccc23)c1=O] |
| 187 | 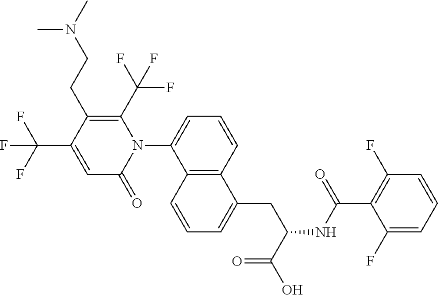 [SMILES: CN(C)CCc1c(C(F)(F)F)cc(=O)n(-c2cccc3c(C[C@H](NC(=O)c4c(F)cccc4F)C(=O)O)cccc23)c1C(F)(F)F] |
| 188 | 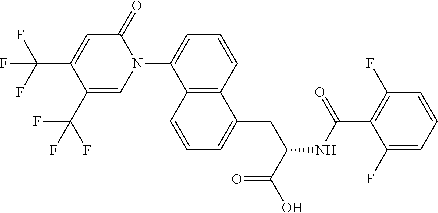 [SMILES: O=C(N[C@@H](Cc1cccc2c(-n3cc(C(F)(F)F)c(C(F)(F)F)cc3=O)cccc12)C(=O)O)c1c(F)cccc1F] |
| 189 | 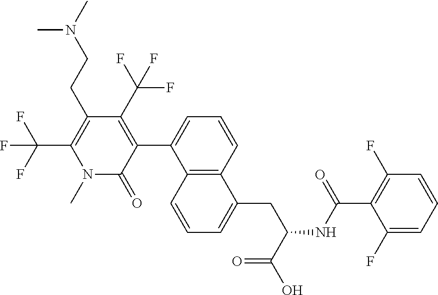 [SMILES: CN(C)CCc1c(C(F)(F)F)c(-c2cccc3c(C[C@H](NC(=O)c4c(F)cccc4F)C(=O)O)cccc23)c(=O)n(C)c1C(F)(F)F] |
| 190 | 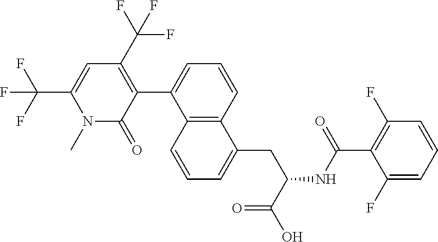 [SMILES: Cn1c(C(F)(F)F)cc(C(F)(F)F)c(-c2cccc3c(C[C@H](NC(=O)c4c(F)cccc4F)C(=O)O)cccc23)c1=O] |
| 191 | 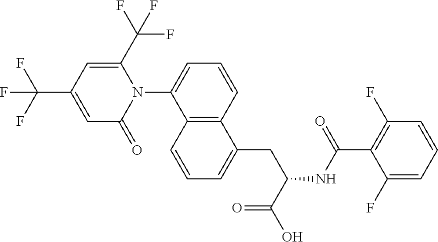 [SMILES: O=C(N[C@@H](Cc1cccc2c(-n3c(C(F)(F)F)cc(C(F)(F)F)cc3=O)cccc12)C(=O)O)c1c(F)cccc1F] |
| 192 | 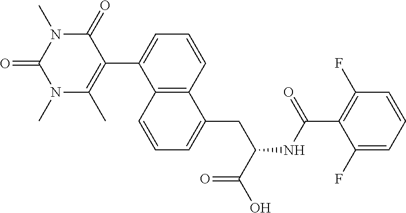 [SMILES: Cc1c(-c2cccc3c(C[C@H](NC(=O)c4c(F)cccc4F)C(=O)O)cccc23)c(=O)n(C)c(=O)n1C] |
| 193 | 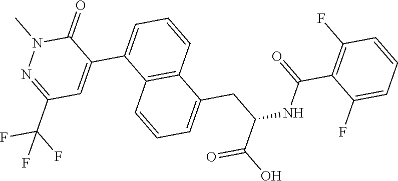 [SMILES: Cn1nc(C(F)(F)F)cc(-c2cccc3c(C[C@H](NC(=O)c4c(F)cccc4F)C(=O)O)cccc23)c1=O] |
| 194 | 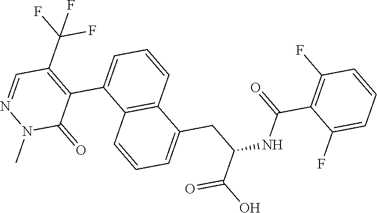 [SMILES: Cn1ncc(C(F)(F)F)c(-c2cccc3c(C[C@H](NC(=O)c4c(F)cccc4F)C(=O)O)cccc23)c1=O] |
| 195 | 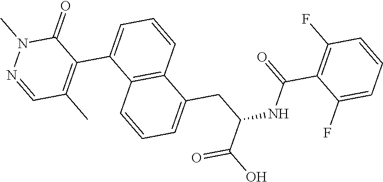 [SMILES: Cc1cnn(C)c(=O)c1-c1cccc2c(C[C@H](NC(=O)c3c(F)cccc3F)C(=O)O)cccc12] |
| 196 | 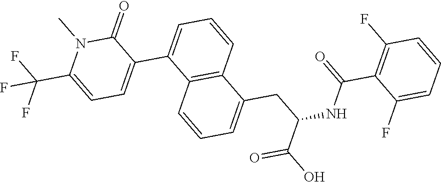 [SMILES: Cn1c(C(F)(F)F)ccc(-c2cccc3c(C[C@H](NC(=O)c4c(F)cccc4F)C(=O)O)cccc23)c1=O] |
| 197 | 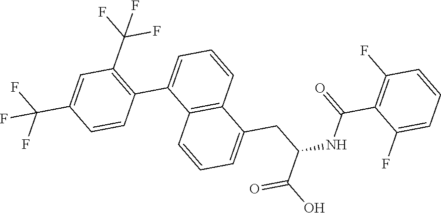 [SMILES: O=C(N[C@@H](Cc1cccc2c(-c3ccc(C(F)(F)F)cc3C(F)(F)F)cccc12)C(=O)O)c1c(F)cccc1F] |
| 198 | 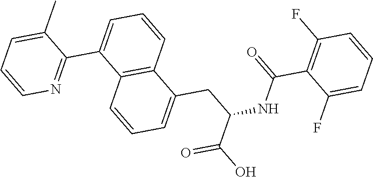 [SMILES: Cc1cccnc1-c1cccc2c(C[C@H](NC(=O)c3c(F)cccc3F)C(=O)O)cccc12] |
| 199 | 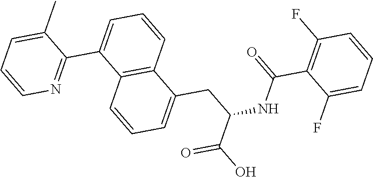 [SMILES: Cc1cccnc1-c1cccc2c(C[C@H](NC(=O)c3c(F)cccc3F)C(=O)O)cccc12] |
| 200 | 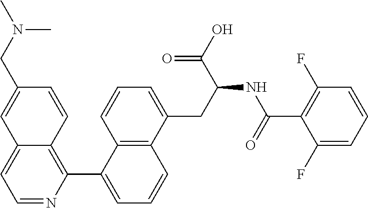 [SMILES: CN(C)Cc1ccc2c(-c3cccc4c(C[C@H](NC(=O)c5c(F)cccc5F)C(=O)O)cccc34)nccc2c1] |
| 201 | 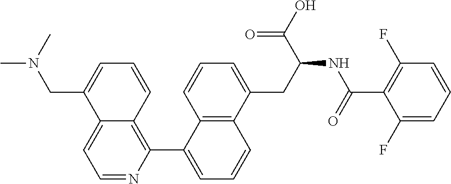 [SMILES: CN(C)Cc1cccc2c(-c3cccc4c(C[C@H](NC(=O)c5c(F)cccc5F)C(=O)O)cccc34)nccc12] |
| 202 | 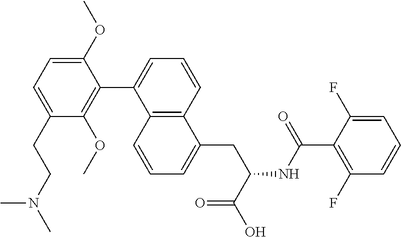 [SMILES: COc1ccc(CCN(C)C)c(OC)c1-c1cccc2c(C[C@H](NC(=O)c3c(F)cccc3F)C(=O)O)cccc12] |
| 203 | 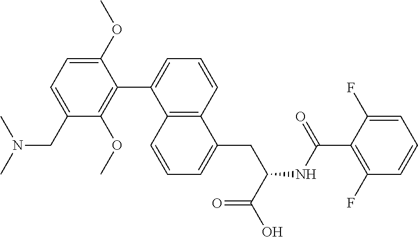 [SMILES: COc1ccc(CN(C)C)c(OC)c1-c1cccc2c(C[C@H](NC(=O)c3c(F)cccc3F)C(=O)O)cccc12] |
| 204 | 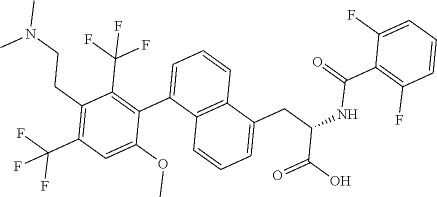 [SMILES: COc1cc(C(F)(F)F)c(CCN(C)C)c(C(F)(F)F)c1-c1cccc2c(C[C@H](NC(=O)c3c(F)cccc3F)C(=O)O)cccc12] |
| 205 | 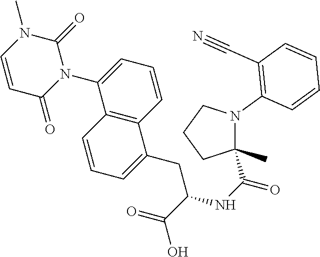 [SMILES: Cn1ccc(=O)n(-c2cccc3c(C[C@H](NC(=O)[C@]4(C)CCCN4c4ccccc4C#N)C(=O)O)cccc23)c1=O] |
| 206 | 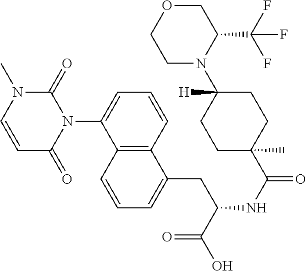 [SMILES: Cn1ccc(=O)n(-c2cccc3c(C[C@H](NC(=O)[C@]4(C)CC[C@@H](N5CCOC[C@@H]5C(F)(F)F)CC4)C(=O)O)cccc23)c1=O] |
| 207 | 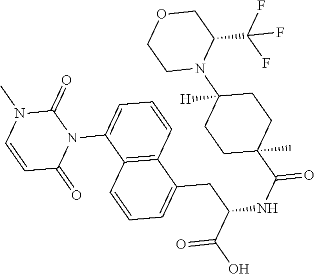 [SMILES: Cn1ccc(=O)n(-c2cccc3c(C[C@H](NC(=O)[C@]4(C)CC[C@H](N5CCOC[C@@H]5C(F)(F)F)CC4)C(=O)O)cccc23)c1=O] |
| 208 | 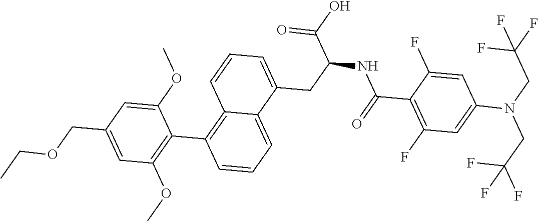 [SMILES: CCOCc1cc(OC)c(-c2cccc3c(C[C@H](NC(=O)c4c(F)cc(N(CC(F)(F)F)CC(F)(F)F)cc4F)C(=O)O)cccc23)c(OC)c1] |
| 209 | 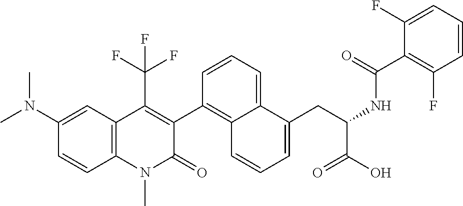 [SMILES: CN(C)c1ccc2c(c1)c(C(F)(F)F)c(-c1cccc3c(C[C@H](NC(=O)c4c(F)cccc4F)C(=O)O)cccc13)c(=O)n2C] |

In some embodiments, the compound or salt of Formulas (I), (I-a), (II) or (III) is selected from Table 2, below, or a pharmaceutically acceptable salt thereof.

TABLE 2

| Compound | |
|---|---|
| No. | Structure |
| 210 | 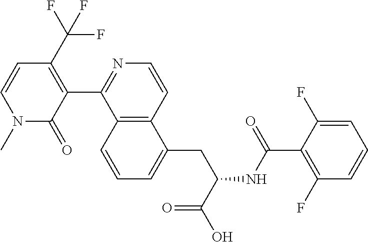 [SMILES: Cn1ccc(C(F)(F)F)c(-c2nccc3c(C[C@H](NC(=O)c4c(F)cccc4F)C(=O)O)cccc23)c1=O] |
| 211 | 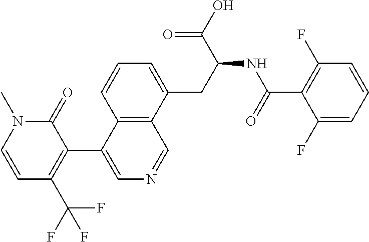 [SMILES: Cn1ccc(C(F)(F)F)c(-c2cncc3c(C[C@H](NC(=O)c4c(F)cccc4F)C(=O)O)cccc23)c1=O] |
| 212 | 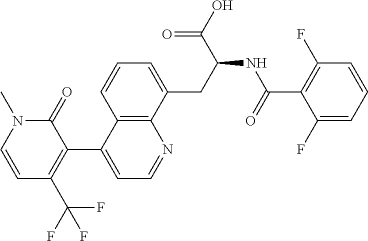 [SMILES: Cn1ccc(C(F)(F)F)c(-c2ccnc3c(C[C@H](NC(=O)c4c(F)cccc4F)C(=O)O)cccc23)c1=O] |
| 213 | 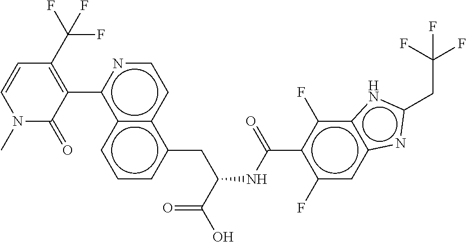 |
| 214 | 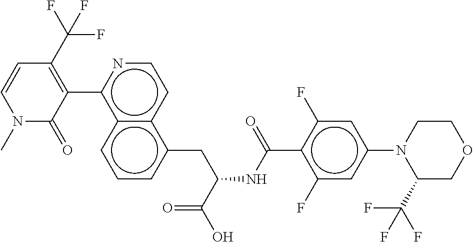 |
| 215 | 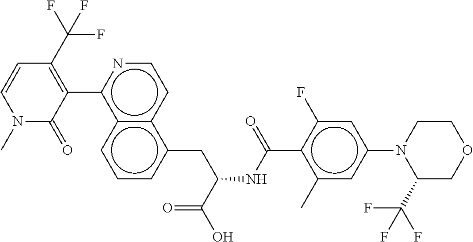 |
| 245 | 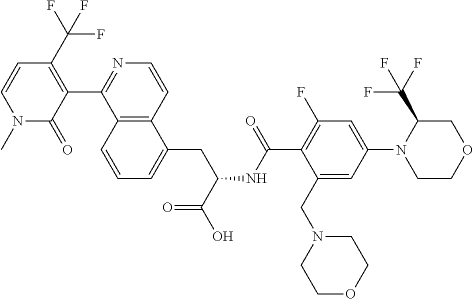 [SMILES: Cn1ccc(C(F)(F)F)c(-c2nccc3c(C[C@H](NC(=O)c4c(F)cc(N5CCOC[C@@H]5C(F)(F)F)cc4CN4CCOCC4)C(=O)O)cccc23)c1=O] |
| 246 | 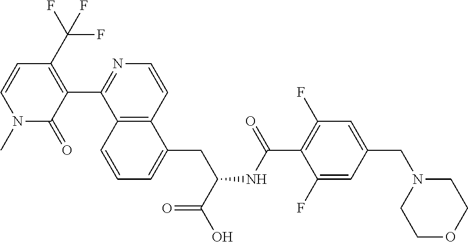 [SMILES: Cn1ccc(C(F)(F)F)c(-c2nccc3c(C[C@H](NC(=O)c4c(F)cc(CN5CCOCC5)cc4F)C(=O)O)cccc23)c1=O] |
| 237 | 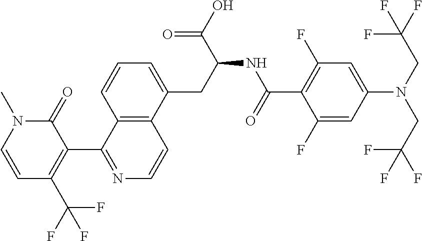 [SMILES: Cn1ccc(C(F)(F)F)c(-c2nccc3c(C[C@H](NC(=O)c4c(F)cc(N(CC(F)(F)F)CC(F)(F)F)cc4F)C(=O)O)cccc23)c1=O] |
| 248 | 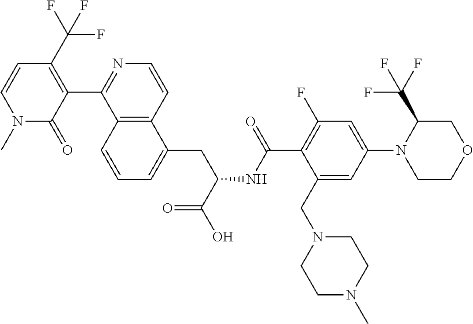 [SMILES: CN1CCN(Cc2cc(N3CCOC[C@@H]3C(F)(F)F)cc(F)c2C(=O)N[C@@H](Cc2cccc3c(-c4c(C(F)(F)F)ccn(C)c4=O)nccc23)C(=O)O)CC1] |
| 249 | 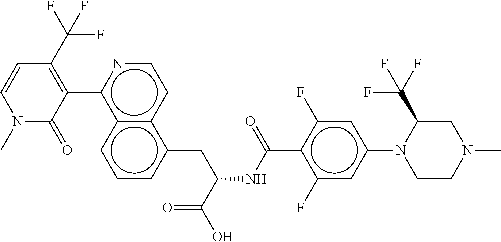 |
| 250 | 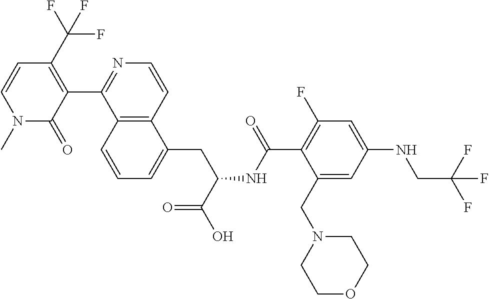 [SMILES: Cn1ccc(C(F)(F)F)c(-c2nccc3c(C[C@H](NC(=O)c4c(F)cc(NCC(F)(F)F)cc4CN4CCOCC4)C(=O)O)cccc23)c1=O] |
| 216 | 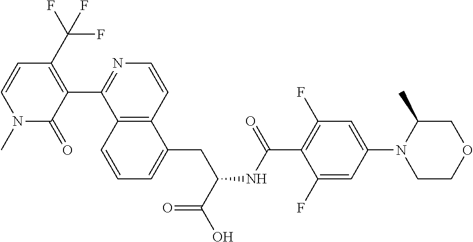 [SMILES: C[C@H]1COCCN1c1cc(F)c(C(=O)N[C@@H](Cc2cccc3c(-c4c(C(F)(F)F)ccn(C)c4=O)nccc23)C(=O)O)c(F)c1] |
| 217 | 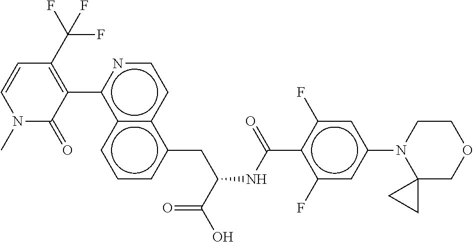 |
| 218 | 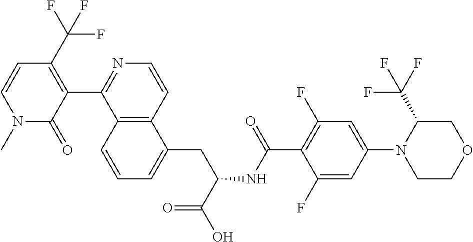 [SMILES: Cn1ccc(C(F)(F)F)c(-c2nccc3c(C[C@H](NC(=O)c4c(F)cc(N5CCOC[C@H]5C(F)(F)F)cc4F)C(=O)O)cccc23)c1=O] |
| 219 | 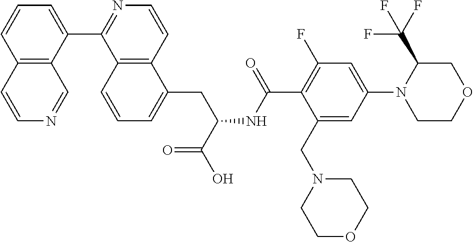 [SMILES: O=C(N[C@@H](Cc1cccc2c(-c3cccc4ccncc34)nccc12)C(=O)O)c1c(F)cc(N2CCOC[C@@H]2C(F)(F)F)cc1CN1CCOCC1] |
| 220 | 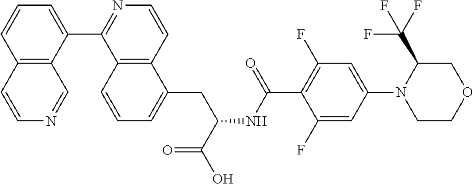 [SMILES: O=C(N[C@@H](Cc1cccc2c(-c3cccc4ccncc34)nccc12)C(=O)O)c1c(F)cc(N2CCOC[C@@H]2C(F)(F)F)cc1F] |
| 221 | 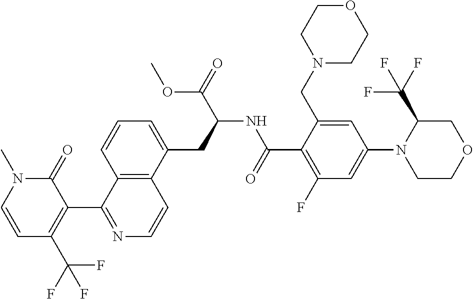 [SMILES: COC(=O)[C@H](Cc1cccc2c(-c3c(C(F)(F)F)ccn(C)c3=O)nccc12)NC(=O)c1c(F)cc(N2CCOC[C@@H]2C(F)(F)F)cc1CN1CCOCC1] |
| 251 | 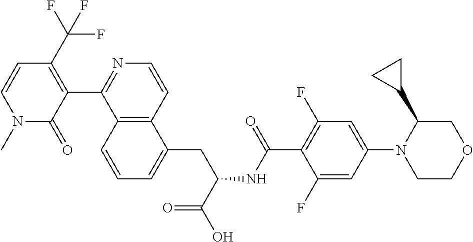 [SMILES: Cn1ccc(C(F)(F)F)c(-c2nccc3c(C[C@H](NC(=O)c4c(F)cc(N5CCOC[C@@H]5C5CC5)cc4F)C(=O)O)cccc23)c1=O] |
| 252 | 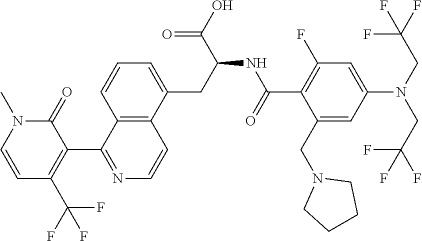 [SMILES: Cn1ccc(C(F)(F)F)c(-c2nccc3c(C[C@H](NC(=O)c4c(F)cc(N(CC(F)(F)F)CC(F)(F)F)cc4CN4CCCC4)C(=O)O)cccc23)c1=O] |
| 253 | 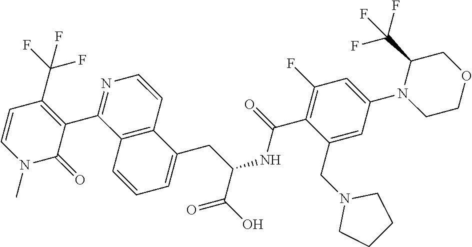 [SMILES: Cn1ccc(C(F)(F)F)c(-c2nccc3c(C[C@H](NC(=O)c4c(F)cc(N5CCOC[C@@H]5C(F)(F)F)cc4CN4CCCC4)C(=O)O)cccc23)c1=O] |
| 254 | 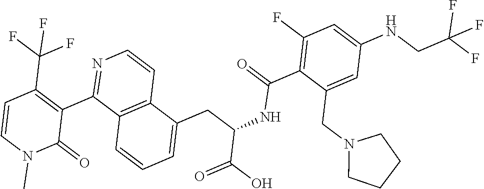 [SMILES: Cn1ccc(C(F)(F)F)c(-c2nccc3c(C[C@H](NC(=O)c4c(F)cc(NCC(F)(F)F)cc4CN4CCCC4)C(=O)O)cccc23)c1=O] |
| 255 | 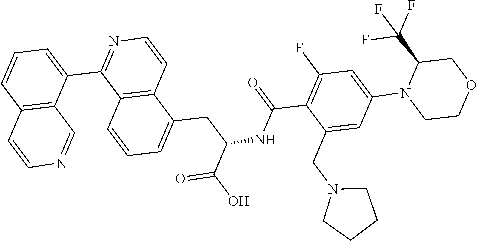 [SMILES: O=C(N[C@@H](Cc1cccc2c(-c3cccc4ccncc34)nccc12)C(=O)O)c1c(F)cc(N2CCOC[C@@H]2C(F)(F)F)cc1CN1CCCC1] |
| 256 | 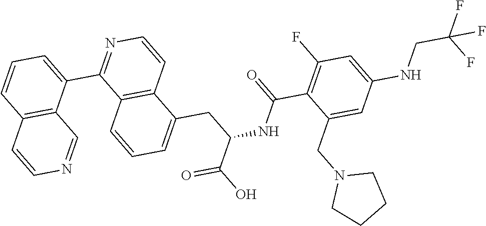 [SMILES: O=C(N[C@@H](Cc1cccc2c(-c3cccc4ccncc34)nccc12)C(=O)O)c1c(F)cc(NCC(F)(F)F)cc1CN1CCCC1] |
| 222 | 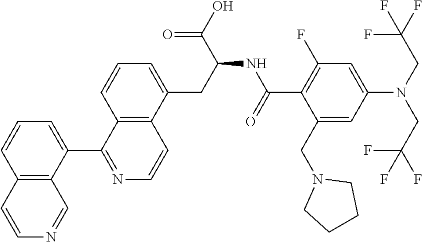 [SMILES: O=C(N[C@@H](Cc1cccc2c(-c3cccc4ccncc34)nccc12)C(=O)O)c1c(F)cc(N(CC(F)(F)F)CC(F)(F)F)cc1CN1CCCC1] |
| 223 | 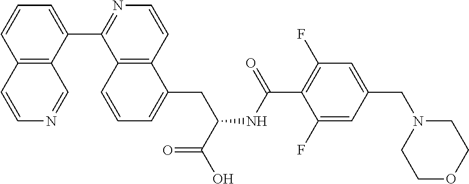 [SMILES: O=C(N[C@@H](Cc1cccc2c(-c3cccc4ccncc34)nccc12)C(=O)O)c1c(F)cc(CN2CCOCC2)cc1F] |
| 224 | 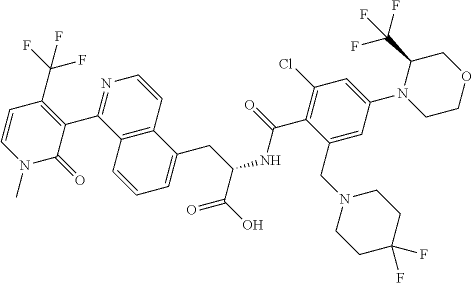 [SMILES: Cn1ccc(C(F)(F)F)c(-c2nccc3c(C[C@H](NC(=O)c4c(Cl)cc(N5CCOC[C@@H]5C(F)(F)F)cc4CN4CCC(F)(F)CC4)C(=O)O)cccc23)c1=O] |
| 225 | 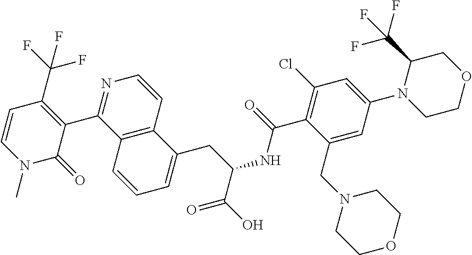 [SMILES: Cn1ccc(C(F)(F)F)c(-c2nccc3c(C[C@H](NC(=O)c4c(Cl)cc(N5CCOC[C@@H]5C(F)(F)F)cc4CN4CCOCC4)C(=O)O)cccc23)c1=O] |
| 226 | 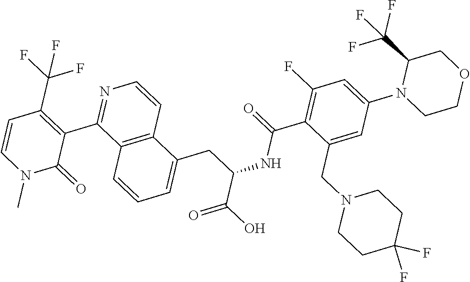 [SMILES: Cn1ccc(C(F)(F)F)c(-c2nccc3c(C[C@H](NC(=O)c4c(F)cc(N5CCOC[C@@H]5C(F)(F)F)cc4CN4CCC(F)(F)CC4)C(=O)O)cccc23)c1=O] |
| 227 | 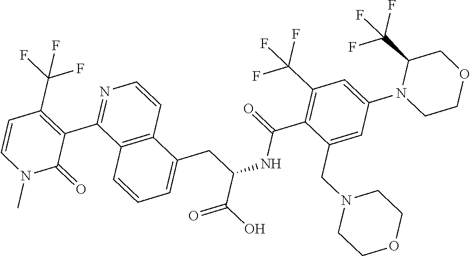 [SMILES: Cn1ccc(C(F)(F)F)c(-c2nccc3c(C[C@H](NC(=O)c4c(CN5CCOCC5)cc(N5CCOC[C@@H]5C(F)(F)F)cc4C(F)(F)F)C(=O)O)cccc23)c1=O] |
| 257 | 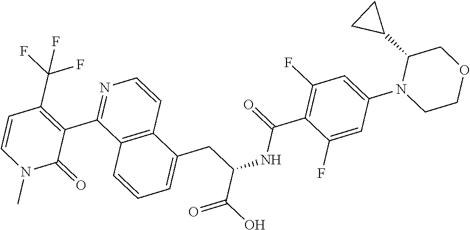 [SMILES: Cn1ccc(C(F)(F)F)c(-c2nccc3c(C[C@H](NC(=O)c4c(F)cc(N5CCOC[C@H]5C5CC5)cc4F)C(=O)O)cccc23)c1=O] |
| 258 | 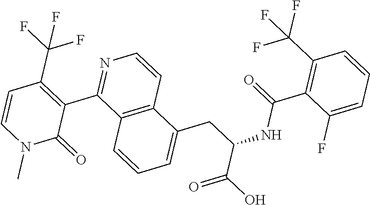 [SMILES: Cn1ccc(C(F)(F)F)c(-c2nccc3c(C[C@H](NC(=O)c4c(F)cccc4C(F)(F)F)C(=O)O)cccc23)c1=O] |
| 259 | 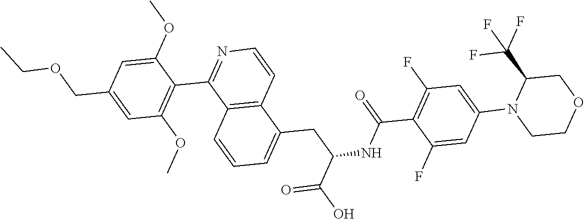 [SMILES: CCOCc1cc(OC)c(-c2nccc3c(C[C@H](NC(=O)c4c(F)cc(N5CCOC[C@@H]5C(F)(F)F)cc4F)C(=O)O)cccc23)c(OC)c1] |
| 260 | 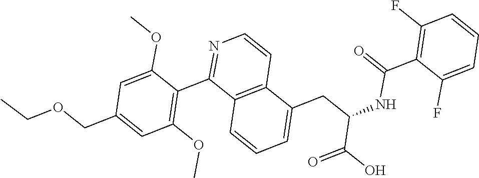 [SMILES: CCOCc1cc(OC)c(-c2nccc3c(C[C@H](NC(=O)c4c(F)cccc4F)C(=O)O)cccc23)c(OC)c1] |
| 261 | 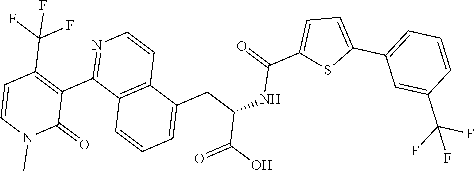 [SMILES: Cn1ccc(C(F)(F)F)c(-c2nccc3c(C[C@H](NC(=O)c4ccc(-c5cccc(C(F)(F)F)c5)s4)C(=O)O)cccc23)c1=O] |
| 262 | 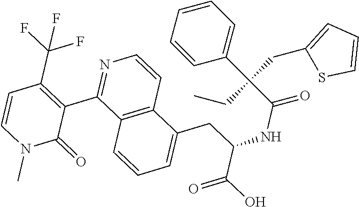 [SMILES: CC[C@](Cc1cccs1)(C(=O)N[C@@H](Cc1cccc2c(-c3c(C(F)(F)F)ccn(C)c3=O)nccc12)C(=O)O)c1ccccc1] |
| 263 | 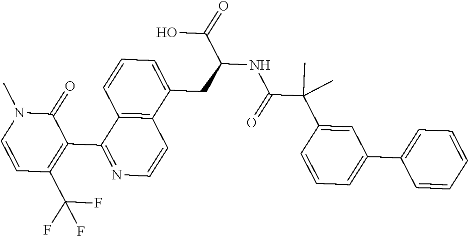 [SMILES: Cn1ccc(C(F)(F)F)c(-c2nccc3c(C[C@H](NC(=O)C(C)(C)c4cccc(-c5ccccc5)c4)C(=O)O)cccc23)c1=O] |
| 228 | 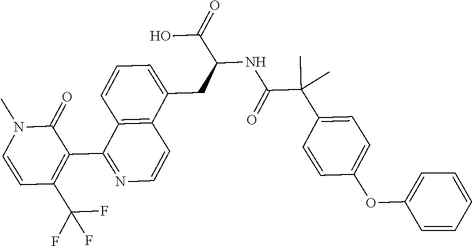 [SMILES: Cn1ccc(C(F)(F)F)c(-c2nccc3c(C[C@H](NC(=O)C(C)(C)c4ccc(Oc5ccccc5)cc4)C(=O)O)cccc23)c1=O] |
| 229 | 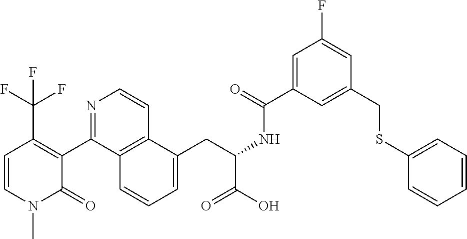 [SMILES: Cn1ccc(C(F)(F)F)c(-c2nccc3c(C[C@H](NC(=O)c4cc(F)cc(CSc5ccccc5)c4)C(=O)O)cccc23)c1=O] |
| 230 | 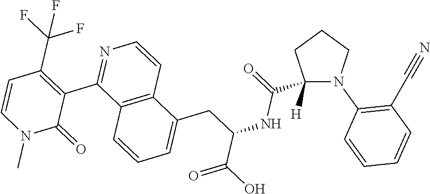 [SMILES: Cn1ccc(C(F)(F)F)c(-c2nccc3c(C[C@H](NC(=O)[C@@H]4CCCN4c4ccccc4C#N)C(=O)O)cccc23)c1=O] |
| 231 | 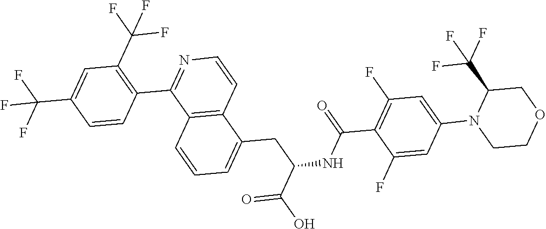 [SMILES: O=C(N[C@@H](Cc1cccc2c(-c3ccc(C(F)(F)F)cc3C(F)(F)F)nccc12)C(=O)O)c1c(F)cc(N2CCOC[C@@H]2C(F)(F)F)cc1F] |
| 232 | 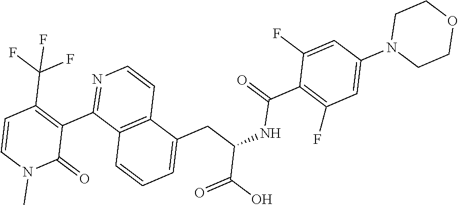 [SMILES: Cn1ccc(C(F)(F)F)c(-c2nccc3c(C[C@H](NC(=O)c4c(F)cc(N5CCOCC5)cc4F)C(=O)O)cccc23)c1=O] |
| 233 | 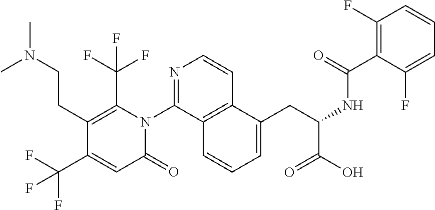 [SMILES: CN(C)CCc1c(C(F)(F)F)cc(=O)n(-c2nccc3c(C[C@H](NC(=O)c4c(F)cccc4F)C(=O)O)cccc23)c1C(F)(F)F] |
| 234 | 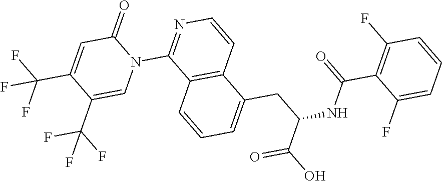 [SMILES: O=C(N[C@@H](Cc1cccc2c(-n3cc(C(F)(F)F)c(C(F)(F)F)cc3=O)nccc12)C(=O)O)c1c(F)cccc1F] |
| 264 | 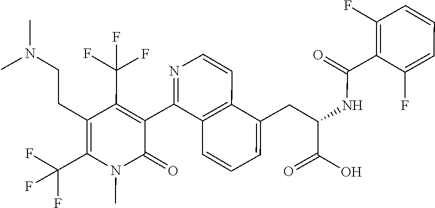 [SMILES: CN(C)CCc1c(C(F)(F)F)c(-c2nccc3c(C[C@H](NC(=O)c4c(F)cccc4F)C(=O)O)cccc23)c(=O)n(C)c1C(F)(F)F] |
| 265 | 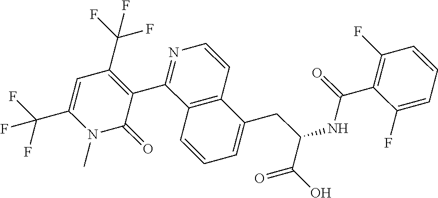 [SMILES: Cn1c(C(F)(F)F)cc(C(F)(F)F)c(-c2nccc3c(C[C@H](NC(=O)c4c(F)cccc4F)C(=O)O)cccc23)c1=O] |
| 266 | 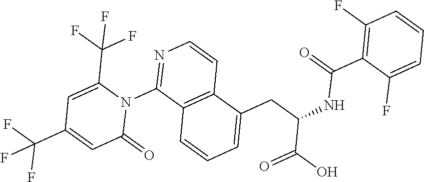 [SMILES: O=C(N[C@@H](Cc1cccc2c(-n3c(C(F)(F)F)cc(C(F)(F)F)cc3=O)nccc12)C(=O)O)c1c(F)cccc1F] |
| 267 | 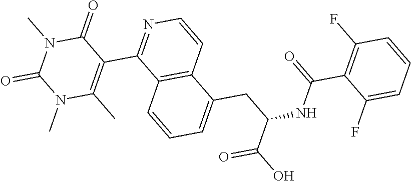 [SMILES: Cc1c(-c2nccc3c(C[C@H](NC(=O)c4c(F)cccc4F)C(=O)O)cccc23)c(=O)n(C)c(=O)n1C] |
| 268 | 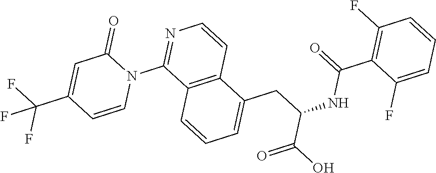 [SMILES: O=C(N[C@@H](Cc1cccc2c(-n3ccc(C(F)(F)F)cc3=O)nccc12)C(=O)O)c1c(F)cccc1F] |
| 269 | 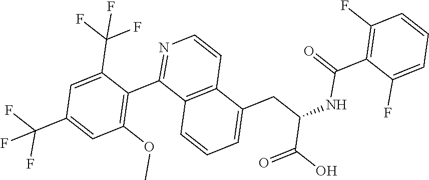 [SMILES: COc1cc(C(F)(F)F)cc(C(F)(F)F)c1-c1nccc2c(C[C@H](NC(=O)c3c(F)cccc3F)C(=O)O)cccc12] |
| 270 | 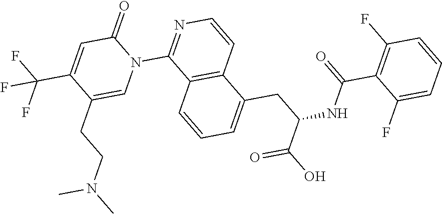 [SMILES: CN(C)CCc1cn(-c2nccc3c(C[C@H](NC(=O)c4c(F)cccc4F)C(=O)O)cccc23)c(=O)cc1C(F)(F)F] |
| 235 | 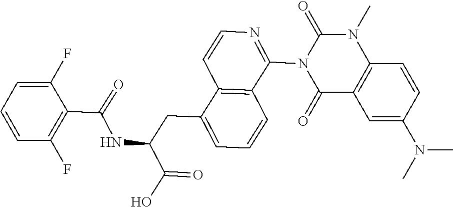 [SMILES: CN(C)c1ccc2c(c1)c(=O)n(-c1nccc3c(C[C@H](NC(=O)c4c(F)cccc4F)C(=O)O)cccc13)c(=O)n2C] |
| 236 | 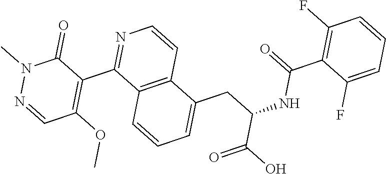 [SMILES: COc1cnn(C)c(=O)c1-c1nccc2c(C[C@H](NC(=O)c3c(F)cccc3F)C(=O)O)cccc12] |
| 237 | 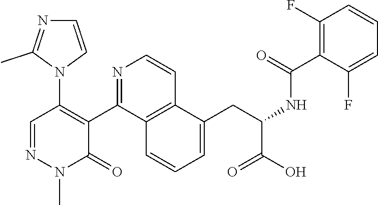 [SMILES: Cc1nccn1-c1cnn(C)c(=O)c1-c1nccc2c(C[C@H](NC(=O)c3c(F)cccc3F)C(=O)O)cccc12] |
| 238 | 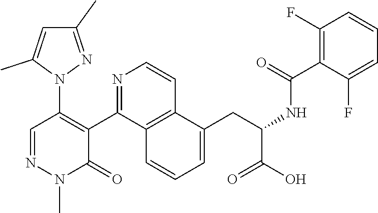 [SMILES: Cc1cc(C)n(-c2cnn(C)c(=O)c2-c2nccc3c(C[C@H](NC(=O)c4c(F)cccc4F)C(=O)O)cccc23)n1] |
| 239 | 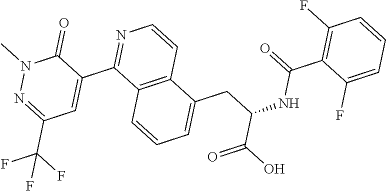 [SMILES: Cn1nc(C(F)(F)F)cc(-c2nccc3c(C[C@H](NC(=O)c4c(F)cccc4F)C(=O)O)cccc23)c1=O] |
| 240 | 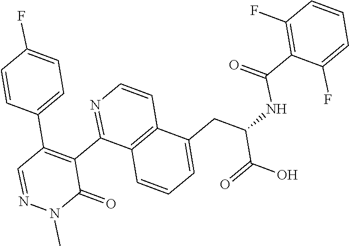 [SMILES: Cn1ncc(-c2ccc(F)cc2)c(-c2nccc3c(C[C@H](NC(=O)c4c(F)cccc4F)C(=O)O)cccc23)c1=O] |
| 271 | 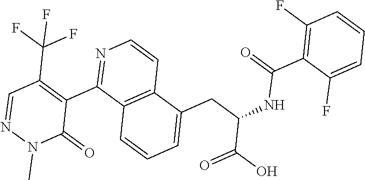 [SMILES: Cn1ncc(C(F)(F)F)c(-c2nccc3c(C[C@H](NC(=O)c4c(F)cccc4F)C(=O)O)cccc23)c1=O] |
| 272 | 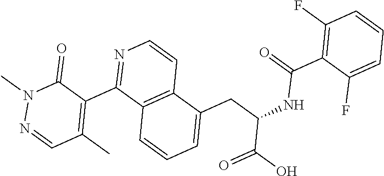 [SMILES: Cc1cnn(C)c(=O)c1-c1nccc2c(C[C@H](NC(=O)c3c(F)cccc3F)C(=O)O)cccc12] |
| 273 | 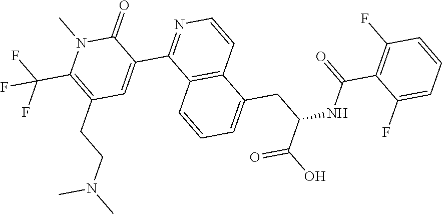 [SMILES: CN(C)CCc1cc(-c2nccc3c(C[C@H](NC(=O)c4c(F)cccc4F)C(=O)O)cccc23)c(=O)n(C)c1C(F)(F)F] |
| 274 | 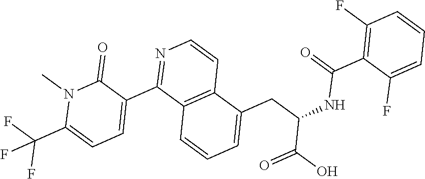 [SMILES: Cn1c(C(F)(F)F)ccc(-c2nccc3c(C[C@H](NC(=O)c4c(F)cccc4F)C(=O)O)cccc23)c1=O] |
| 275 | 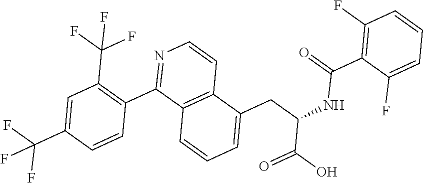 [SMILES: O=C(N[C@@H](Cc1cccc2c(-c3ccc(C(F)(F)F)cc3C(F)(F)F)nccc12)C(=O)O)c1c(F)cccc1F] |
| 276 | 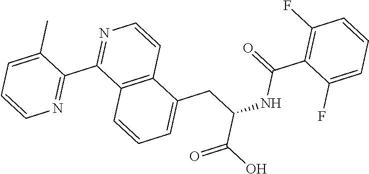 [SMILES: Cc1cccnc1-c1nccc2c(C[C@H](NC(=O)c3c(F)cccc3F)C(=O)O)cccc12] |
| 277 | 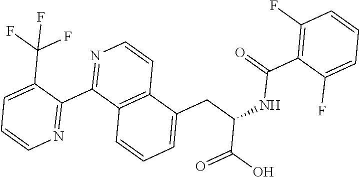 [SMILES: O=C(N[C@@H](Cc1cccc2c(-c3ncccc3C(F)(F)F)nccc12)C(=O)O)c1c(F)cccc1F] |
| 241 | 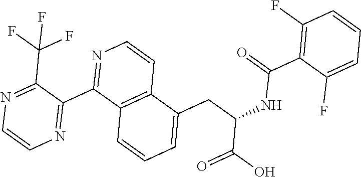 [SMILES: O=C(N[C@@H](Cc1cccc2c(-c3nccnc3C(F)(F)F)nccc12)C(=O)O)c1c(F)cccc1F] |
| 242 | 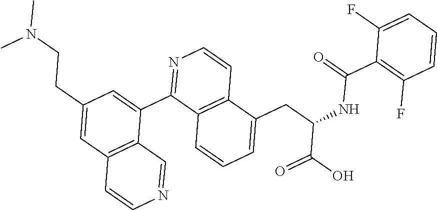 [SMILES: CN(C)CCc1cc(-c2nccc3c(C[C@H](NC(=O)c4c(F)cccc4F)C(=O)O)cccc23)c2cnccc2c1] |
| 243 | 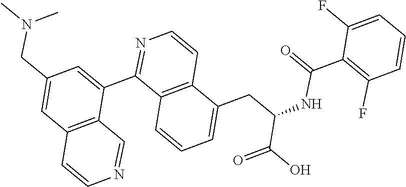 [SMILES: CN(C)Cc1cc(-c2nccc3c(C[C@H](NC(=O)c4c(F)cccc4F)C(=O)O)cccc23)c2cnccc2c1] |
| 244 | 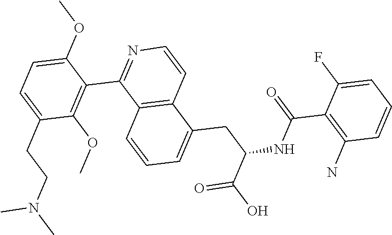 [SMILES: COc1ccc(CCN(C)C)c(OC)c1-c1nccc2c(C[C@H](NC(=O)c3c(N)cccc3F)C(=O)O)cccc12] |
| 278 | 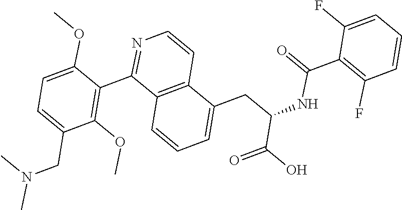 [SMILES: COc1ccc(CN(C)C)c(OC)c1-c1nccc2c(C[C@H](NC(=O)c3c(F)cccc3F)C(=O)O)cccc12] |
| 279 | 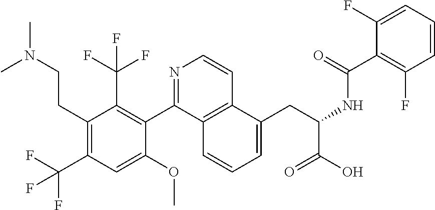 [SMILES: COc1cc(C(F)(F)F)c(CCN(C)C)c(C(F)(F)F)c1-c1nccc2c(C[C@H](NC(=O)c3c(F)cccc3F)C(=O)O)cccc12] |
| 280 | 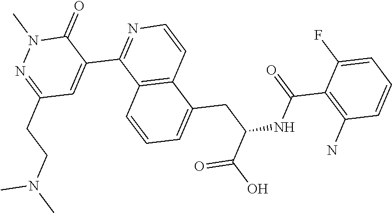 [SMILES: CN(C)CCc1cc(-c2nccc3c(C[C@H](NC(=O)c4c(N)cccc4F)C(=O)O)cccc23)c(=O)n(C)n1] |

In some embodiments, the compound or salt of Formulas (I), (I-a), (II) or (III) is selected from Table 3, below, or a pharmaceutically acceptable salt thereof.

TABLE 3

| Compound | |
|---|---|
| No. | Structure |
| 281* | 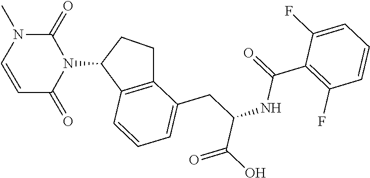 [SMILES: Cn1ccc(=O)n([C@@H]2CCc3c(C[C@H](NC(=O)c4c(F)cccc4F)C(=O)O)cccc32)c1=O] |
| 282* | 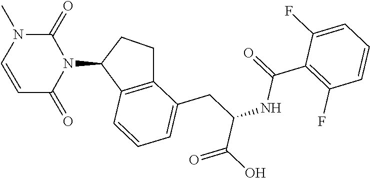 [SMILES: Cn1ccc(=O)n([C@H]2CCc3c(C[C@H](NC(=O)c4c(F)cccc4F)C(=O)O)cccc32)c1=O] |
| 283* | 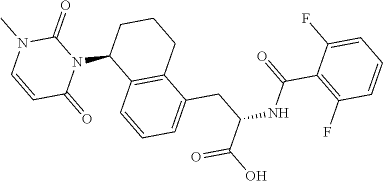 [SMILES: Cn1ccc(=O)n([C@H]2CCCc3c(C[C@H](NC(=O)c4c(F)cccc4F)C(=O)O)cccc32)c1=O] |
| 284* | 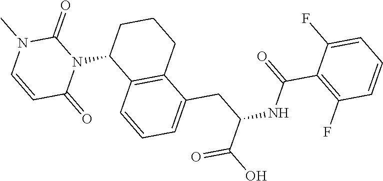 [SMILES: Cn1ccc(=O)n([C@@H]2CCCc3c(C[C@H](NC(=O)c4c(F)cccc4F)C(=O)O)cccc32)c1=O] |
| 285* | 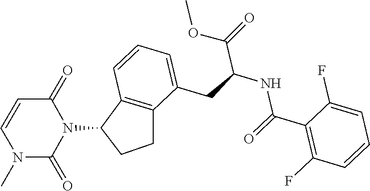 [SMILES: COC(=O)[C@H](Cc1cccc2c1CC[C@@H]2n1c(=O)ccn(C)c1=O)NC(=O)c1c(F)cccc1F] |
| 286* | 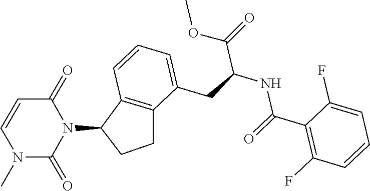 [SMILES: COC(=O)[C@H](Cc1cccc2c1CC[C@H]2n1c(=O)ccn(C)c1=O)NC(=O)c1c(F)cccc1F] |
| 287 | 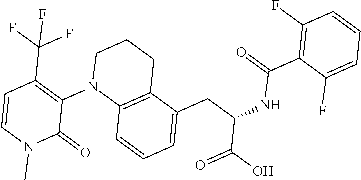 [SMILES: Cn1ccc(C(F)(F)F)c(N2CCCc3c(C[C@H](NC(=O)c4c(F)cccc4F)C(=O)O)cccc32)c1=O] |
| 288 | 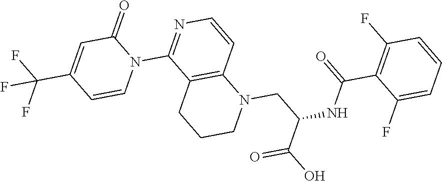 [SMILES: O=C(N[C@@H](CN1CCCc2c1ccnc2-n1ccc(C(F)(F)F)cc1=O)C(=O)O)c1c(F)cccc1F] |
| 289 | 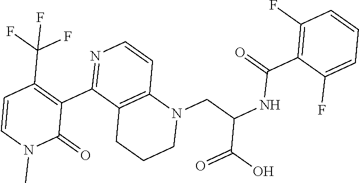 [SMILES: Cn1ccc(C(F)(F)F)c(-c2nccc3c2CCCN3CC(NC(=O)c2c(F)cccc2F)C(=O)O)c1=O] |

*Denotes a racemic compound;
**Denotes a single isomer with unknown absolute stereochemistry.

In some embodiments, the compound or salt of Formulas (I), (I-a), (II) or (III) is selected from a compound of Table 4, below, or a pharmaceutically acceptable salt thereof.

TABLE 4
Structures of Selected Compounds 291-469

| Compound | |
|---|---|
| No | Structure |
| 291 | 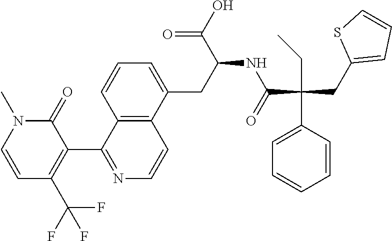 [SMILES: CC[C@@](Cc1cccs1)(C(=O)N[C@@H](Cc1cccc2c(-c3c(C(F)(F)F)ccn(C)c3=O)nccc12)C(=O)O)c1ccccc1] |
| 292 | 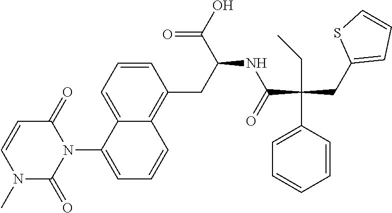 [SMILES: CC[C@@](Cc1cccs1)(C(=O)N[C@@H](Cc1cccc2c(-n3c(=O)ccn(C)c3=O)cccc12)C(=O)O)c1ccccc1] |
| 293 | 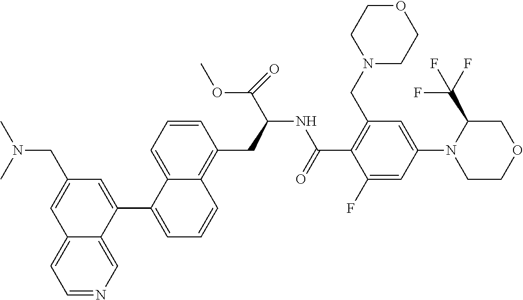 [SMILES: COC(=O)[C@H](Cc1cccc2c(-c3cc(CN(C)C)cc4ccncc34)cccc12)NC(=O)c1c(F)cc(N2CCOC[C@@H]2C(F)(F)F)cc1CN1CCOCC1] |
| 294 | 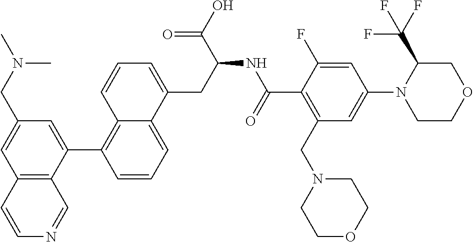 [SMILES: CN(C)Cc1cc(-c2cccc3c(C[C@H](NC(=O)c4c(F)cc(N5CCOC[C@@H]5C(F)(F)F)cc4CN4CCOCC4)C(=O)O)cccc23)c2cnccc2c1] |
| 295 | 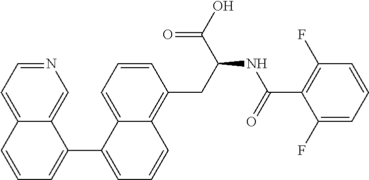 [SMILES: O=C(N[C@@H](Cc1cccc2c(-c3cccc4ccncc34)cccc12)C(=O)O)c1c(F)cccc1F] |
| 296 | 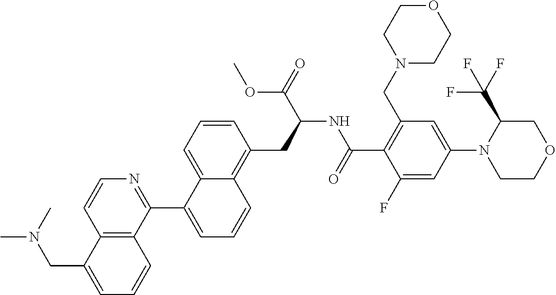 [SMILES: COC(=O)[C@H](Cc1cccc2c(-c3nccc4c(CN(C)C)cccc34)cccc12)NC(=O)c1c(F)cc(N2CCOC[C@@H]2C(F)(F)F)cc1CN1CCOCC1] |
| 297 | 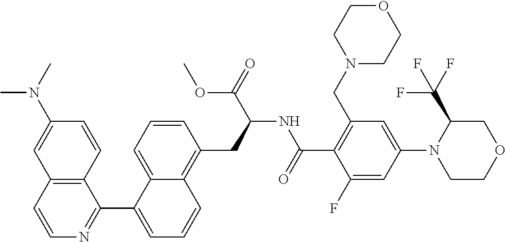 [SMILES: COC(=O)[C@H](Cc1cccc2c(-c3nccc4cc(N(C)C)ccc34)cccc12)NC(=O)c1c(F)cc(N2CCOC[C@@H]2C(F)(F)F)cc1CN1CCOCC1] |
| 298 | 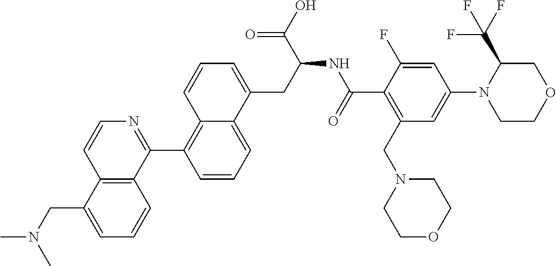 [SMILES: CN(C)Cc1cccc2c(-c3cccc4c(C[C@H](NC(=O)c5c(F)cc(N6CCOC[C@@H]6C(F)(F)F)cc5CN5CCOCC5)C(=O)O)cccc34)nccc12] |
| 299 | 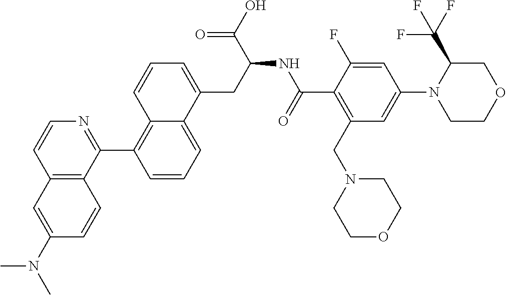 [SMILES: CN(C)c1ccc2c(-c3cccc4c(C[C@H](NC(=O)c5c(F)cc(N6CCOC[C@@H]6C(F)(F)F)cc5CN5CCOCC5)C(=O)O)cccc34)nccc2c1] |
| 300 | 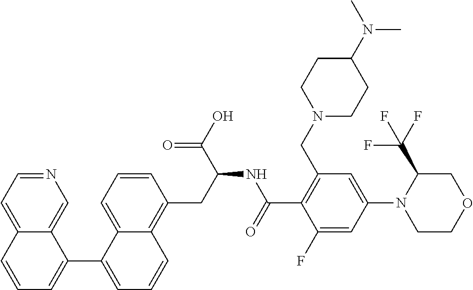 [SMILES: CN(C)C1CCN(Cc2cc(N3CCOC[C@@H]3C(F)(F)F)cc(F)c2C(=O)N[C@@H](Cc2cccc3c(-c4cccc5ccncc45)cccc23)C(=O)O)CC1] |
| 301 | 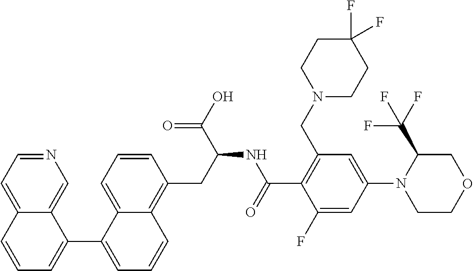 [SMILES: O=C(N[C@@H](Cc1cccc2c(-c3cccc4ccncc34)cccc12)C(=O)O)c1c(F)cc(N2CCOC[C@@H]2C(F)(F)F)cc1CN1CCC(F)(F)CC1] |
| 302 | 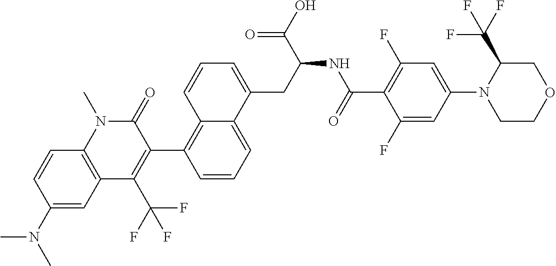 [SMILES: CN(C)c1ccc2c(c1)c(C(F)(F)F)c(-c1cccc3c(C[C@H](NC(=O)c4c(F)cc(N5CCOC[C@@H]5C(F)(F)F)cc4F)C(=O)O)cccc13)c(=O)n2C] |
| 303 | 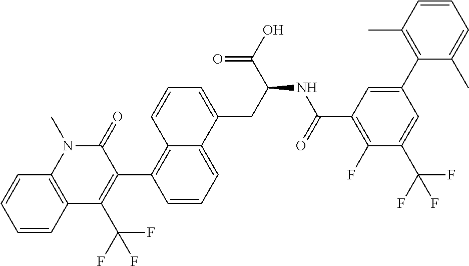 [SMILES: Cc1cccc(C)c1-c1cc(C(=O)N[C@@H](Cc2cccc3c(-c4c(C(F)(F)F)c5ccccc5n(C)c4=O)cccc23)C(=O)O)c(F)c(C(F)(F)F)c1] |
| 304 | 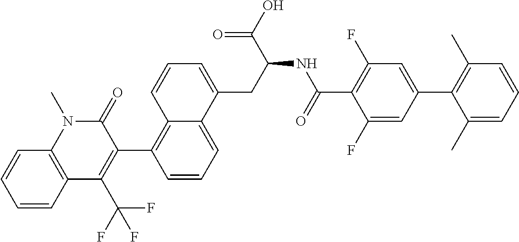 [SMILES: Cc1cccc(C)c1-c1cc(F)c(C(=O)N[C@@H](Cc2cccc3c(-c4c(C(F)(F)F)c5ccccc5n(C)c4=O)cccc23)C(=O)O)c(F)c1] |
| 305 | 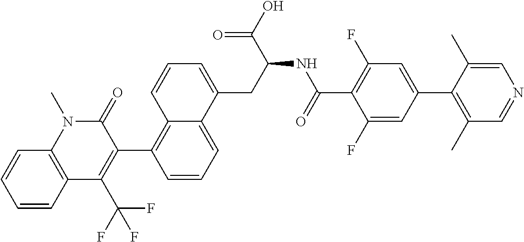 [SMILES: Cc1cncc(C)c1-c1cc(F)c(C(=O)N[C@@H](Cc2cccc3c(-c4c(C(F)(F)F)c5ccccc5n(C)c4=O)cccc23)C(=O)O)c(F)c1] |
| 306 | 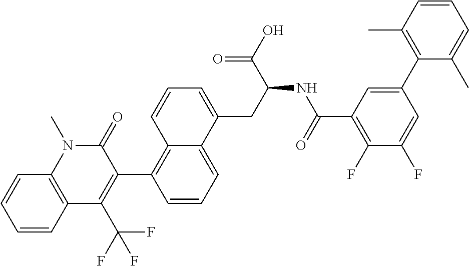 [SMILES: Cc1cccc(C)c1-c1cc(F)c(F)c(C(=O)N[C@@H](Cc2cccc3c(-c4c(C(F)(F)F)c5ccccc5n(C)c4=O)cccc23)C(=O)O)c1] |
| 307 | 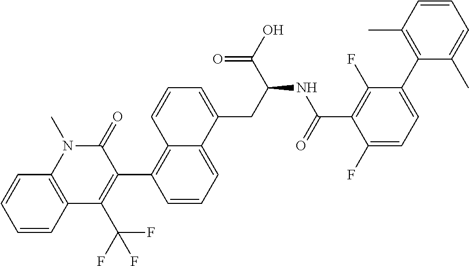 [SMILES: Cc1cccc(C)c1-c1ccc(F)c(C(=O)N[C@@H](Cc2cccc3c(-c4c(C(F)(F)F)c5ccccc5n(C)c4=O)cccc23)C(=O)O)c1F] |
| 308 | 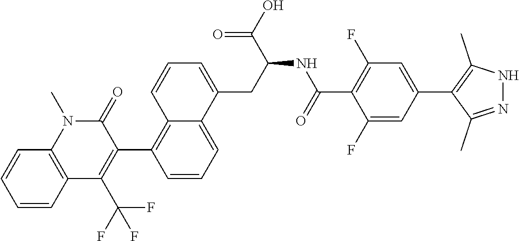 [SMILES: Cc1n[nH]c(C)c1-c1cc(F)c(C(=O)N[C@@H](Cc2cccc3c(-c4c(C(F)(F)F)c5ccccc5n(C)c4=O)cccc23)C(=O)O)c(F)c1] |
| 309 | 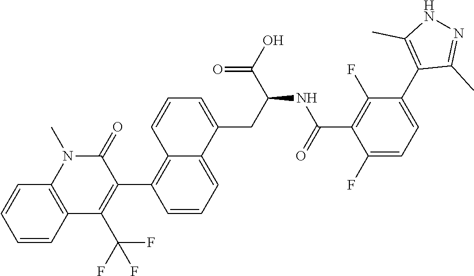 [SMILES: Cc1n[nH]c(C)c1-c1ccc(F)c(C(=O)N[C@@H](Cc2cccc3c(-c4c(C(F)(F)F)c5ccccc5n(C)c4=O)cccc23)C(=O)O)c1F] |
| 310 | 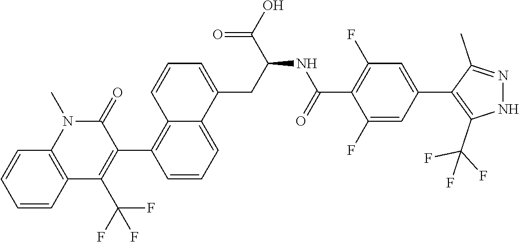 [SMILES: Cc1n[nH]c(C(F)(F)F)c1-c1cc(F)c(C(=O)N[C@@H](Cc2cccc3c(-c4c(C(F)(F)F)c5ccccc5n(C)c4=O)cccc23)C(=O)O)c(F)c1] |
| 311 | 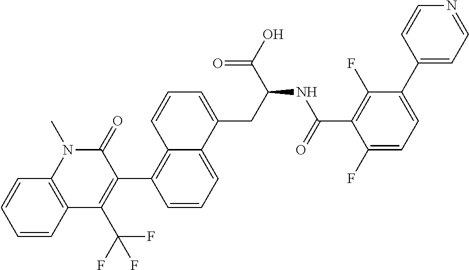 [SMILES: Cn1c(=O)c(-c2cccc3c(C[C@H](NC(=O)c4c(F)ccc(-c5ccncc5)c4F)C(=O)O)cccc23)c(C(F)(F)F)c2ccccc21] |
| 312 | 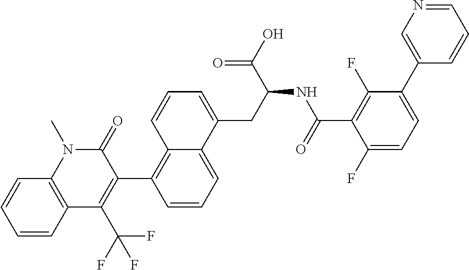 [SMILES: Cn1c(=O)c(-c2cccc3c(C[C@H](NC(=O)c4c(F)ccc(-c5cccnc5)c4F)C(=O)O)cccc23)c(C(F)(F)F)c2ccccc21] |
| 313 | 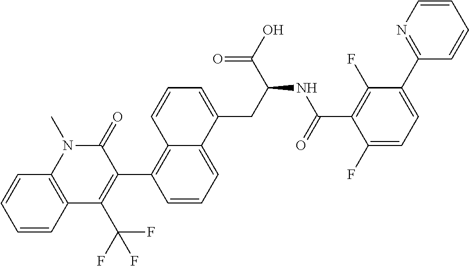 [SMILES: Cn1c(=O)c(-c2cccc3c(C[C@H](NC(=O)c4c(F)ccc(-c5ccccn5)c4F)C(=O)O)cccc23)c(C(F)(F)F)c2ccccc21] |
| 314 | 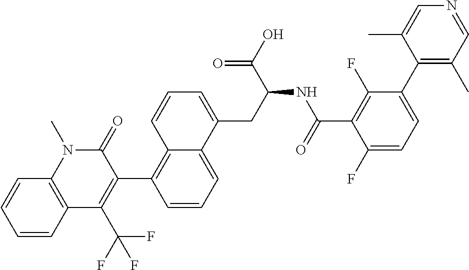 [SMILES: Cc1cncc(C)c1-c1ccc(F)c(C(=O)N[C@@H](Cc2cccc3c(-c4c(C(F)(F)F)c5ccccc5n(C)c4=O)cccc23)C(=O)O)c1F] |
| 315 | 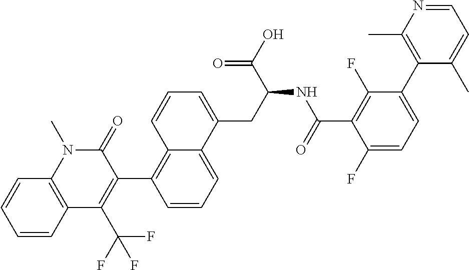 [SMILES: Cc1ccnc(C)c1-c1ccc(F)c(C(=O)N[C@@H](Cc2cccc3c(-c4c(C(F)(F)F)c5ccccc5n(C)c4=O)cccc23)C(=O)O)c1F] |
| 316 | 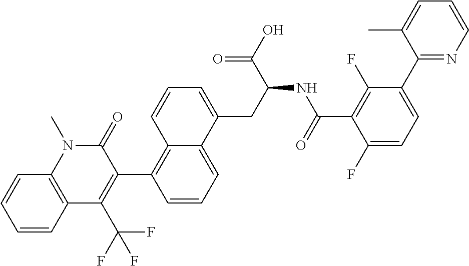 [SMILES: Cc1cccnc1-c1ccc(F)c(C(=O)N[C@@H](Cc2cccc3c(-c4c(C(F)(F)F)c5ccccc5n(C)c4=O)cccc23)C(=O)O)c1F] |
| 317 | 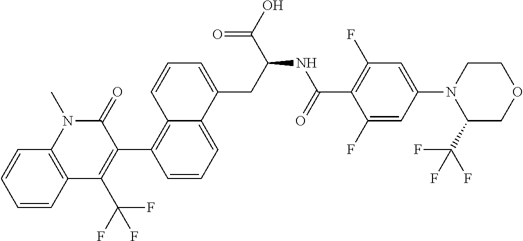 [SMILES: Cn1c(=O)c(-c2cccc3c(C[C@H](NC(=O)c4c(F)cc(N5CCOC[C@@H]5C(F)(F)F)cc4F)C(=O)O)cccc23)c(C(F)(F)F)c2ccccc21] |
| 318 | 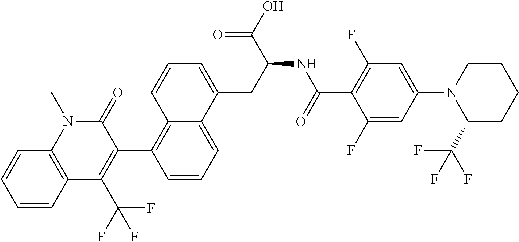 [SMILES: Cn1c(=O)c(-c2cccc3c(C[C@H](NC(=O)c4c(F)cc(N5CCCC[C@@H]5C(F)(F)F)cc4F)C(=O)O)cccc23)c(C(F)(F)F)c2ccccc21] |
| 319 | 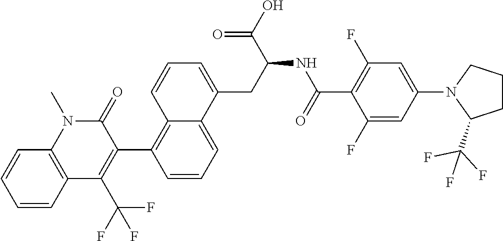 [SMILES: Cn1c(=O)c(-c2cccc3c(C[C@H](NC(=O)c4c(F)cc(N5CCC[C@@H]5C(F)(F)F)cc4F)C(=O)O)cccc23)c(C(F)(F)F)c2ccccc21] |
| 320 | 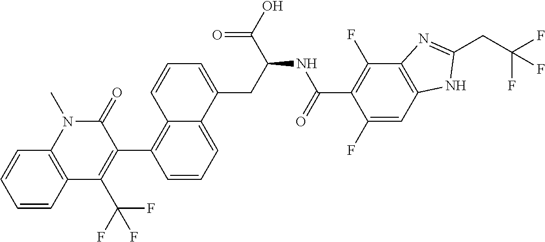 [SMILES: Cn1c(=O)c(-c2cccc3c(C[C@H](NC(=O)c4c(F)cc5[nH]c(CC(F)(F)F)nc5c4F)C(=O)O)cccc23)c(C(F)(F)F)c2ccccc21] |
| 321 | 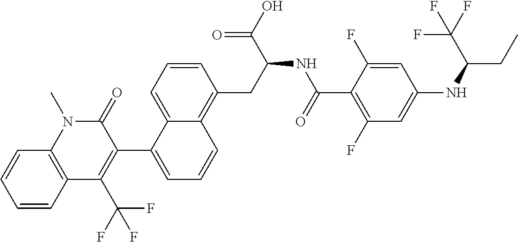 [SMILES: CC[C@@H](Nc1cc(F)c(C(=O)N[C@@H](Cc2cccc3c(-c4c(C(F)(F)F)c5ccccc5n(C)c4=O)cccc23)C(=O)O)c(F)c1)C(F)(F)F] |
| 322 | 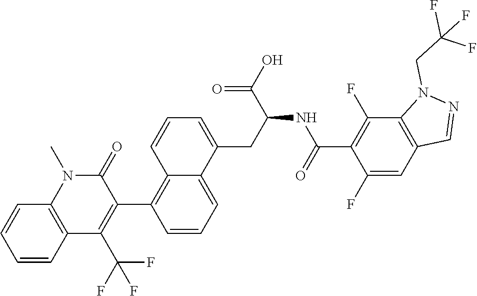 [SMILES: Cn1c(=O)c(-c2cccc3c(C[C@H](NC(=O)c4c(F)cc5cnn(CC(F)(F)F)c5c4F)C(=O)O)cccc23)c(C(F)(F)F)c2ccccc21] |
| 323 | 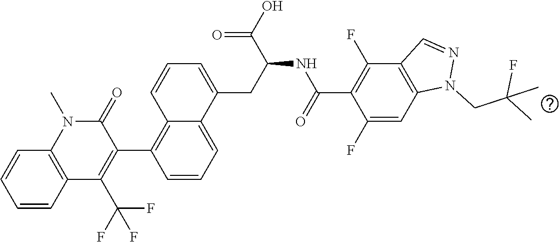 [SMILES: Cn1c(=O)c(-c2cccc3c(C[C@H](NC(=O)c4c(F)cc5c(cnn5CC(C)(C)F)c4F)C(=O)O)cccc23)c(C(F)(F)F)c2ccccc21] |
| 324 | 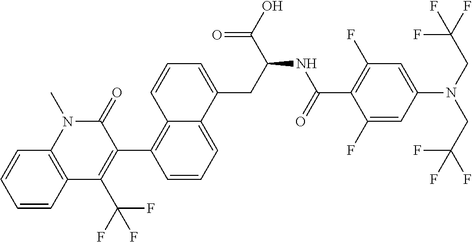 [SMILES: Cn1c(=O)c(-c2cccc3c(C[C@H](NC(=O)c4c(F)cc(N(CC(F)(F)F)CC(F)(F)F)cc4F)C(=O)O)cccc23)c(C(F)(F)F)c2ccccc21] |
| 325 | 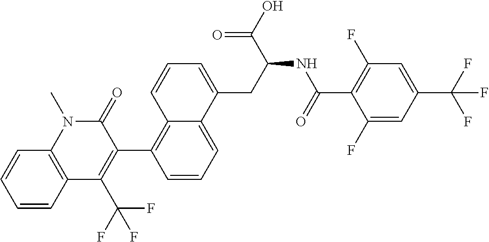 [SMILES: Cn1c(=O)c(-c2cccc3c(C[C@H](NC(=O)c4c(F)cc(C(F)(F)F)cc4F)C(=O)O)cccc23)c(C(F)(F)F)c2ccccc21] |
| 326 | 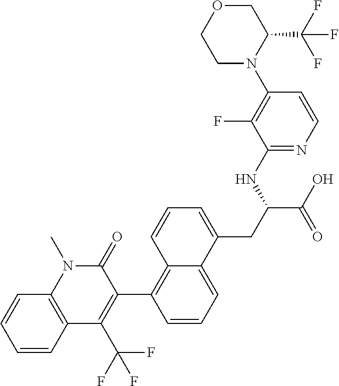 [SMILES: Cn1c(=O)c(-c2cccc3c(C[C@H](Nc4nccc(N5CCOC[C@@H]5C(F)(F)F)c4F)C(=O)O)cccc23)c(C(F)(F)F)c2ccccc21] |
| 327 | 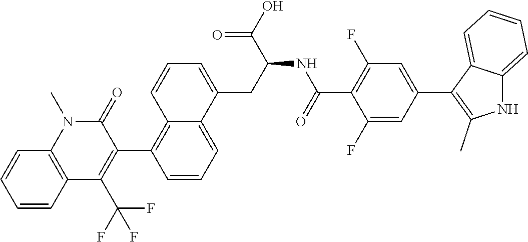 [SMILES: Cc1[nH]c2ccccc2c1-c1cc(F)c(C(=O)N[C@@H](Cc2cccc3c(-c4c(C(F)(F)F)c5ccccc5n(C)c4=O)cccc23)C(=O)O)c(F)c1] |
| 328 | 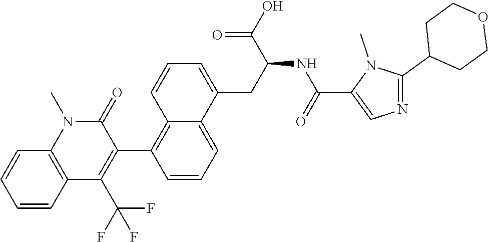 [SMILES: Cn1c(C(=O)N[C@@H](Cc2cccc3c(-c4c(C(F)(F)F)c5ccccc5n(C)c4=O)cccc23)C(=O)O)cnc1C1CCOCC1] |
| 329 | 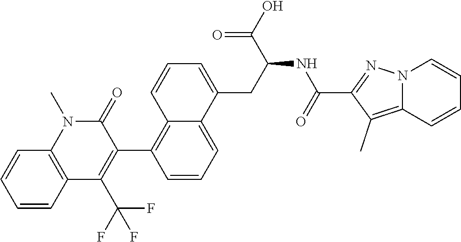 [SMILES: Cc1c(C(=O)N[C@@H](Cc2cccc3c(-c4c(C(F)(F)F)c5ccccc5n(C)c4=O)cccc23)C(=O)O)nn2ccccc12] |
| 330 | 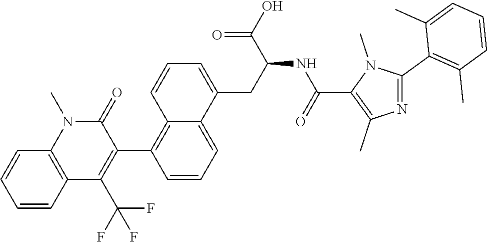 [SMILES: Cc1cccc(C)c1-c1nc(C)c(C(=O)N[C@@H](Cc2cccc3c(-c4c(C(F)(F)F)c5ccccc5n(C)c4=O)cccc23)C(=O)O)n1C] |
| 331 | 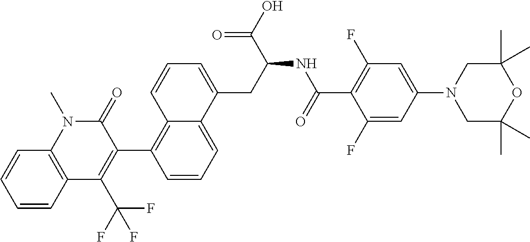 [SMILES: Cn1c(=O)c(-c2cccc3c(C[C@H](NC(=O)c4c(F)cc(N5CC(C)(C)OC(C)(C)C5)cc4F)C(=O)O)cccc23)c(C(F)(F)F)c2ccccc21] |
| 332 | 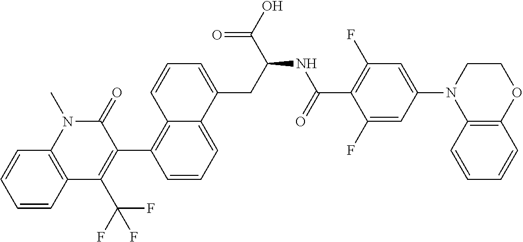 [SMILES: Cn1c(=O)c(-c2cccc3c(C[C@H](NC(=O)c4c(F)cc(N5CCOc6ccccc65)cc4F)C(=O)O)cccc23)c(C(F)(F)F)c2ccccc21] |
| 333 | 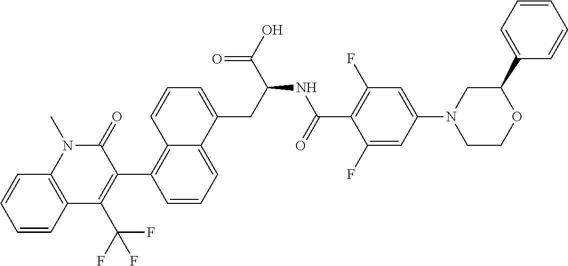 [SMILES: Cn1c(=O)c(-c2cccc3c(C[C@H](NC(=O)c4c(F)cc(N5CCO[C@H](c6ccccc6)C5)cc4F)C(=O)O)cccc23)c(C(F)(F)F)c2ccccc21] |
| 334 | 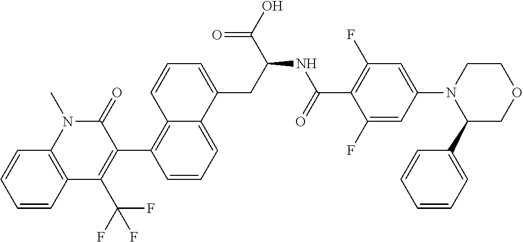 [SMILES: Cn1c(=O)c(-c2cccc3c(C[C@H](NC(=O)c4c(F)cc(N5CCOC[C@H]5c5ccccc5)cc4F)C(=O)O)cccc23)c(C(F)(F)F)c2ccccc21] |
| 335 | 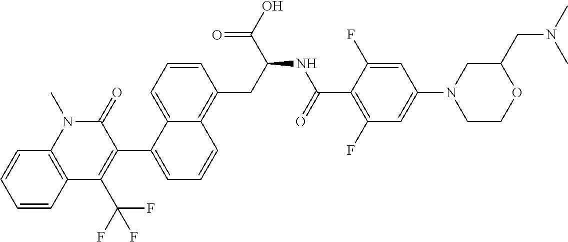 [SMILES: CN(C)CC1CN(c2cc(F)c(C(=O)N[C@@H](Cc3cccc4c(-c5c(C(F)(F)F)c6ccccc6n(C)c5=O)cccc34)C(=O)O)c(F)c2)CCO1] |
| 336 | 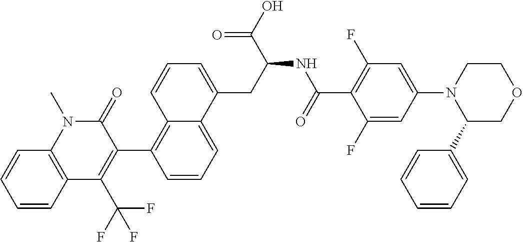 [SMILES: Cn1c(=O)c(-c2cccc3c(C[C@H](NC(=O)c4c(F)cc(N5CCOC[C@@H]5c5ccccc5)cc4F)C(=O)O)cccc23)c(C(F)(F)F)c2ccccc21] |
| 337 | 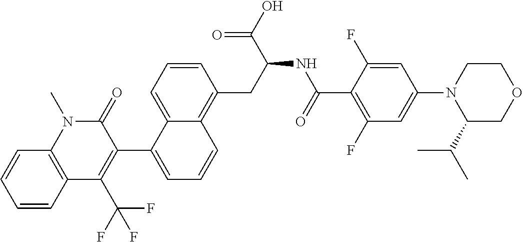 [SMILES: CC(C)[C@H]1COCCN1c1cc(F)c(C(=O)N[C@@H](Cc2cccc3c(-c4c(C(F)(F)F)c5ccccc5n(C)c4=O)cccc23)C(=O)O)c(F)c1] |
| 338 | 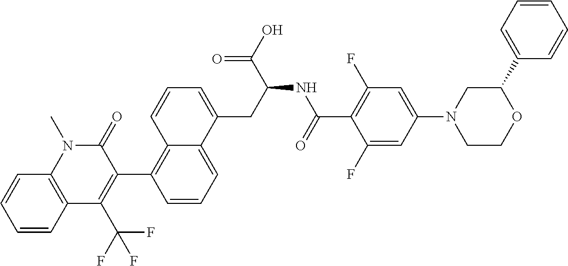 [SMILES: Cn1c(=O)c(-c2cccc3c(C[C@H](NC(=O)c4c(F)cc(N5CCO[C@@H](c6ccccc6)C5)cc4F)C(=O)O)cccc23)c(C(F)(F)F)c2ccccc21] |
| 339 | 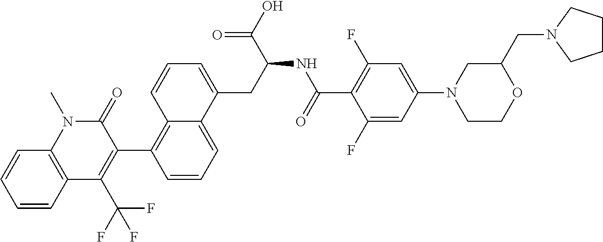 [SMILES: Cn1c(=O)c(-c2cccc3c(C[C@H](NC(=O)c4c(F)cc(N5CCOC(CN6CCCC6)C5)cc4F)C(=O)O)cccc23)c(C(F)(F)F)c2ccccc21] |
| 340 | 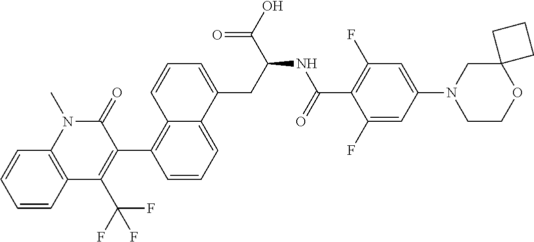 [SMILES: Cn1c(=O)c(-c2cccc3c(C[C@H](NC(=O)c4c(F)cc(N5CCOC6(CCC6)C5)cc4F)C(=O)O)cccc23)c(C(F)(F)F)c2ccccc21] |
| 341 | 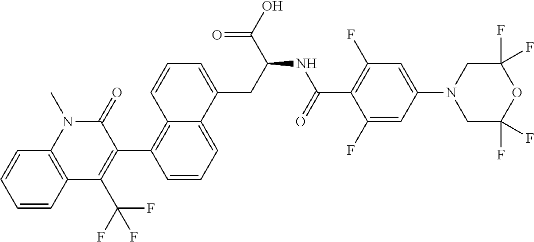 [SMILES: Cn1c(=O)c(-c2cccc3c(C[C@H](NC(=O)c4c(F)cc(N5CC(F)(F)OC(F)(F)C5)cc4F)C(=O)O)cccc23)c(C(F)(F)F)c2ccccc21] |
| 342 | 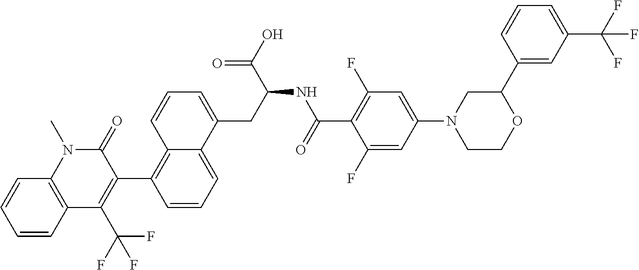 [SMILES: Cn1c(=O)c(-c2cccc3c(C[C@H](NC(=O)c4c(F)cc(N5CCOC(c6cccc(C(F)(F)F)c6)C5)cc4F)C(=O)O)cccc23)c(C(F)(F)F)c2ccccc21] |
| 343 | 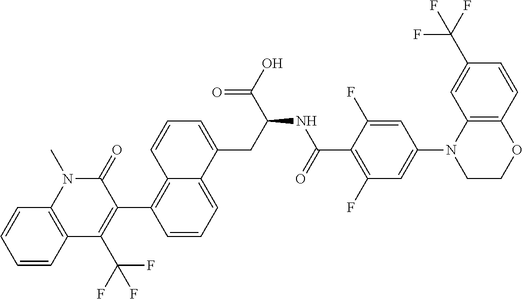 [SMILES: Cn1c(=O)c(-c2cccc3c(C[C@H](NC(=O)c4c(F)cc(N5CCOc6ccc(C(F)(F)F)cc65)cc4F)C(=O)O)cccc23)c(C(F)(F)F)c2ccccc21] |
| 344 | 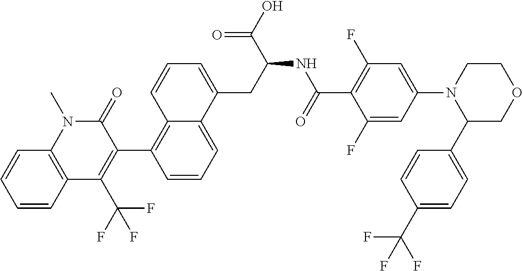 [SMILES: Cn1c(=O)c(-c2cccc3c(C[C@H](NC(=O)c4c(F)cc(N5CCOCC5c5ccc(C(F)(F)F)cc5)cc4F)C(=O)O)cccc23)c(C(F)(F)F)c2ccccc21] |
| 345 | 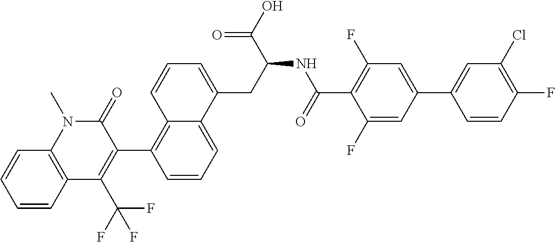 [SMILES: Cn1c(=O)c(-c2cccc3c(C[C@H](NC(=O)c4c(F)cc(-c5ccc(F)c(Cl)c5)cc4F)C(=O)O)cccc23)c(C(F)(F)F)c2ccccc21] |
| 346** | 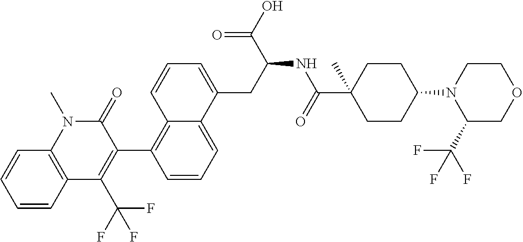 [SMILES: Cn1c(=O)c(-c2cccc3c(C[C@H](NC(=O)[C@]4(C)CC[C@@H](N5CCOC[C@@H]5C(F)(F)F)CC4)C(=O)O)cccc23)c(C(F)(F)F)c2ccccc21] |
| 347** | 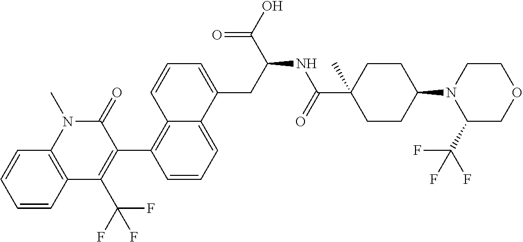 [SMILES: Cn1c(=O)c(-c2cccc3c(C[C@H](NC(=O)[C@]4(C)CC[C@H](N5CCOC[C@@H]5C(F)(F)F)CC4)C(=O)O)cccc23)c(C(F)(F)F)c2ccccc21] |
| 348 | 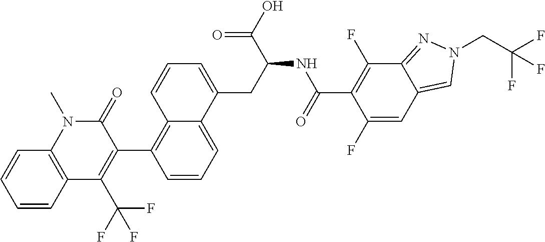 [SMILES: Cn1c(=O)c(-c2cccc3c(C[C@H](NC(=O)c4c(F)cc5cn(CC(F)(F)F)nc5c4F)C(=O)O)cccc23)c(C(F)(F)F)c2ccccc21] |
| 349 | 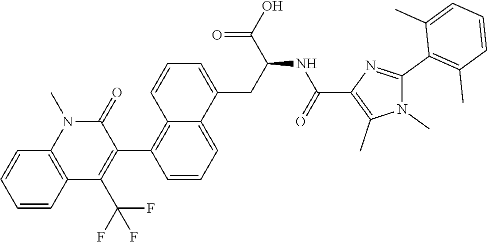 [SMILES: Cc1cccc(C)c1-c1nc(C(=O)N[C@@H](Cc2cccc3c(-c4c(C(F)(F)F)c5ccccc5n(C)c4=O)cccc23)C(=O)O)c(C)n1C] |
| 350 | 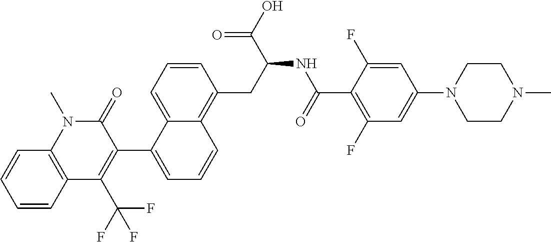 [SMILES: CN1CCN(c2cc(F)c(C(=O)N[C@@H](Cc3cccc4c(-c5c(C(F)(F)F)c6ccccc6n(C)c5=O)cccc34)C(=O)O)c(F)c2)CC1] |
| 351 | 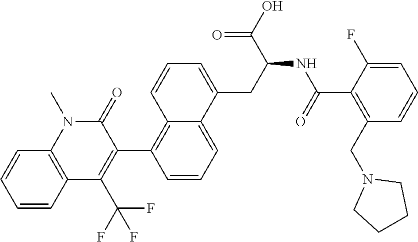 [SMILES: Cn1c(=O)c(-c2cccc3c(C[C@H](NC(=O)c4c(F)cccc4CN4CCCC4)C(=O)O)cccc23)c(C(F)(F)F)c2ccccc21] |
| 352 | 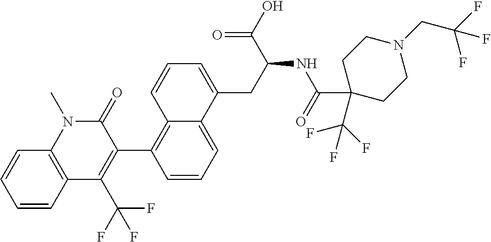 [SMILES: Cn1c(=O)c(-c2cccc3c(C[C@H](NC(=O)C4(C(F)(F)F)CCN(CC(F)(F)F)CC4)C(=O)O)cccc23)c(C(F)(F)F)c2ccccc21] |
| 353 | 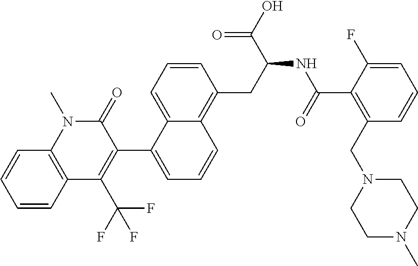 [SMILES: CN1CCN(Cc2cccc(F)c2C(=O)N[C@@H](Cc2cccc3c(-c4c(C(F)(F)F)c5ccccc5n(C)c4=O)cccc23)C(=O)O)CC1] |
| 354 | 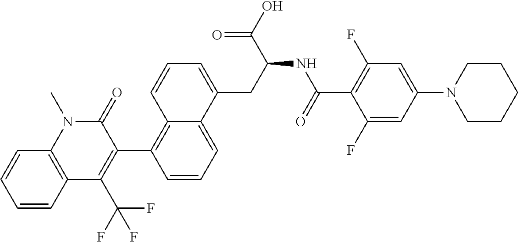 [SMILES: Cn1c(=O)c(-c2cccc3c(C[C@H](NC(=O)c4c(F)cc(N5CCCCC5)cc4F)C(=O)O)cccc23)c(C(F)(F)F)c2ccccc21] |
| 355 | 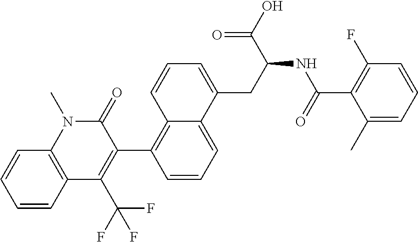 [SMILES: Cc1cccc(F)c1C(=O)N[C@@H](Cc1cccc2c(-c3c(C(F)(F)F)c4ccccc4n(C)c3=O)cccc12)C(=O)O] |
| 356 | 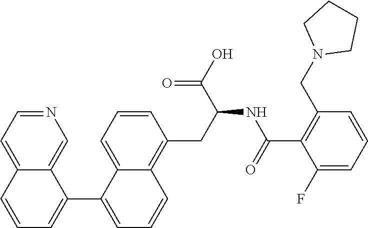 [SMILES: O=C(N[C@@H](Cc1cccc2c(-c3cccc4ccncc34)cccc12)C(=O)O)c1c(F)cccc1CN1CCCC1] |
| 357 | 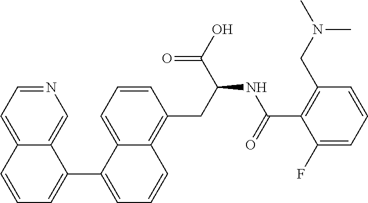 [SMILES: CN(C)Cc1cccc(F)c1C(=O)N[C@@H](Cc1cccc2c(-c3cccc4ccncc34)cccc12)C(=O)O] |
| 358 | 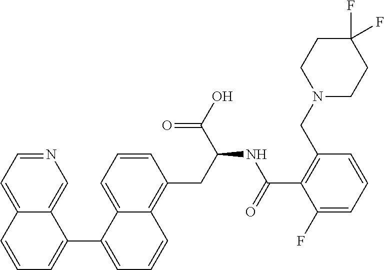 [SMILES: O=C(N[C@@H](Cc1cccc2c(-c3cccc4ccncc34)cccc12)C(=O)O)c1c(F)cccc1CN1CCC(F)(F)CC1] |
| 359 | 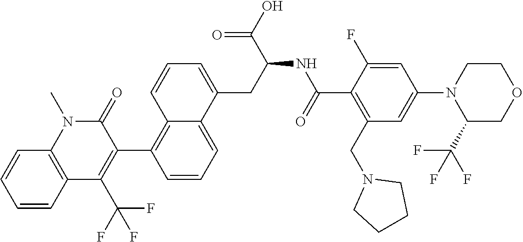 [SMILES: Cn1c(=O)c(-c2cccc3c(C[C@H](NC(=O)c4c(F)cc(N5CCOC[C@@H]5C(F)(F)F)cc4CN4CCCC4)C(=O)O)cccc23)c(C(F)(F)F)c2ccccc21] |
| 360 | 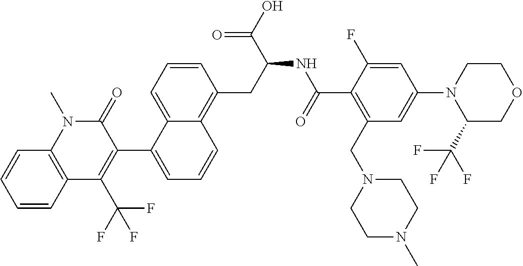 [SMILES: CN1CCN(Cc2cc(N3CCOC[C@@H]3C(F)(F)F)cc(F)c2C(=O)N[C@@H](Cc2cccc3c(-c4c(C(F)(F)F)c5ccccc5n(C)c4=O)cccc23)C(=O)O)CC1] |
| 361 | 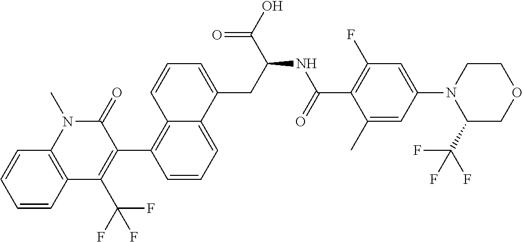 [SMILES: Cc1cc(N2CCOC[C@@H]2C(F)(F)F)cc(F)c1C(=O)N[C@@H](Cc1cccc2c(-c3c(C(F)(F)F)c4ccccc4n(C)c3=O)cccc12)C(=O)O] |
| 362 | 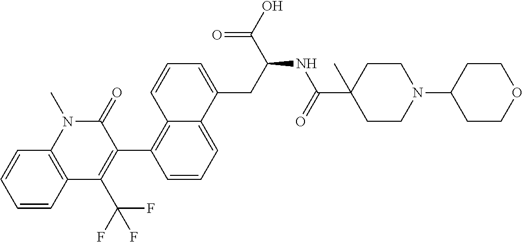 [SMILES: Cn1c(=O)c(-c2cccc3c(C[C@H](NC(=O)C4(C)CCN(C5CCOCC5)CC4)C(=O)O)cccc23)c(C(F)(F)F)c2ccccc21] |
| 363 | 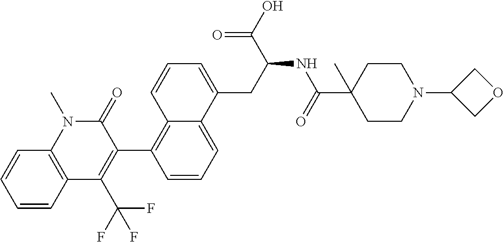 [SMILES: Cn1c(=O)c(-c2cccc3c(C[C@H](NC(=O)C4(C)CCN(C5COC5)CC4)C(=O)O)cccc23)c(C(F)(F)F)c2ccccc21] |
| 364 | 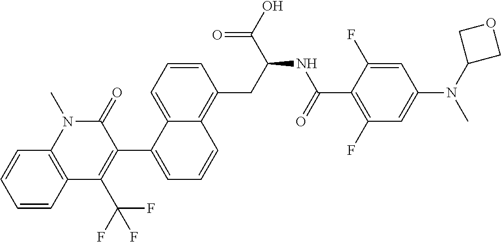 [SMILES: CN(c1cc(F)c(C(=O)N[C@@H](Cc2cccc3c(-c4c(C(F)(F)F)c5ccccc5n(C)c4=O)cccc23)C(=O)O)c(F)c1)C1COC1] |
| 365 | 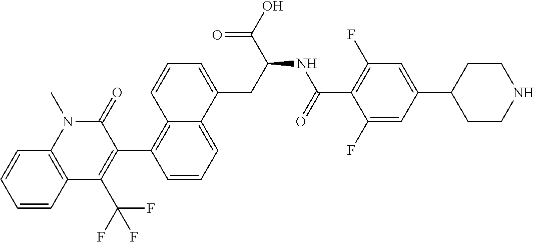 [SMILES: Cn1c(=O)c(-c2cccc3c(C[C@H](NC(=O)c4c(F)cc(C5CCNCC5)cc4F)C(=O)O)cccc23)c(C(F)(F)F)c2ccccc21] |
| 366 | 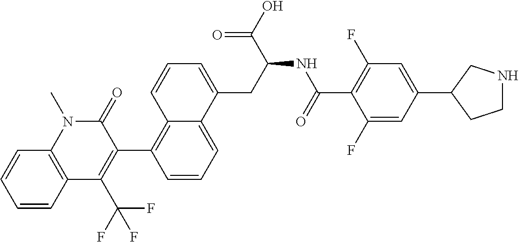 [SMILES: Cn1c(=O)c(-c2cccc3c(C[C@H](NC(=O)c4c(F)cc(C5CCNC5)cc4F)C(=O)O)cccc23)c(C(F)(F)F)c2ccccc21] |
| 367 | 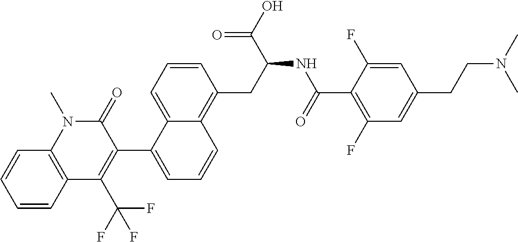 [SMILES: CN(C)CCc1cc(F)c(C(=O)N[C@@H](Cc2cccc3c(-c4c(C(F)(F)F)c5ccccc5n(C)c4=O)cccc23)C(=O)O)c(F)c1] |
| 368 | 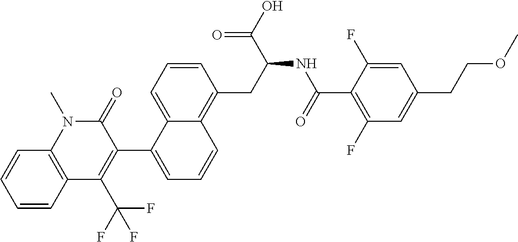 [SMILES: COCCc1cc(F)c(C(=O)N[C@@H](Cc2cccc3c(-c4c(C(F)(F)F)c5ccccc5n(C)c4=O)cccc23)C(=O)O)c(F)c1] |
| 369 | 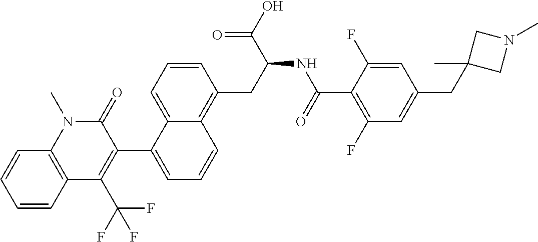 [SMILES: CN1CC(C)(Cc2cc(F)c(C(=O)N[C@@H](Cc3cccc4c(-c5c(C(F)(F)F)c6ccccc6n(C)c5=O)cccc34)C(=O)O)c(F)c2)C1] |
| 370 | 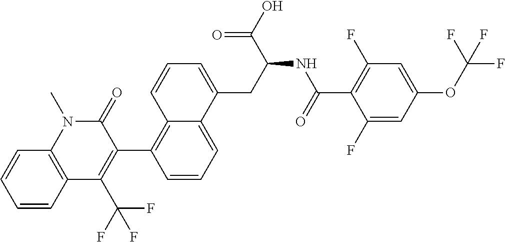 [SMILES: Cn1c(=O)c(-c2cccc3c(C[C@H](NC(=O)c4c(F)cc(OC(F)(F)F)cc4F)C(=O)O)cccc23)c(C(F)(F)F)c2ccccc21] |
| 371 | 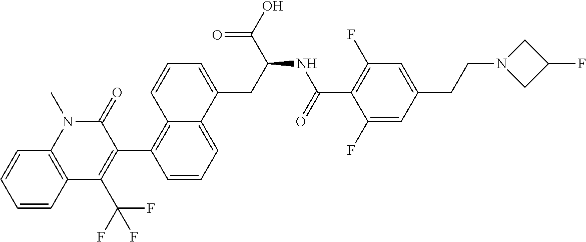 [SMILES: Cn1c(=O)c(-c2cccc3c(C[C@H](NC(=O)c4c(F)cc(CCN5CC(F)C5)cc4F)C(=O)O)cccc23)c(C(F)(F)F)c2ccccc21] |
| 372* | 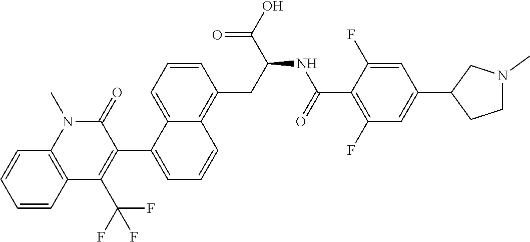 [SMILES: CN1CCC(c2cc(F)c(C(=O)N[C@@H](Cc3cccc4c(-c5c(C(F)(F)F)c6ccccc6n(C)c5=O)cccc34)C(=O)O)c(F)c2)C1] |
| 373 | 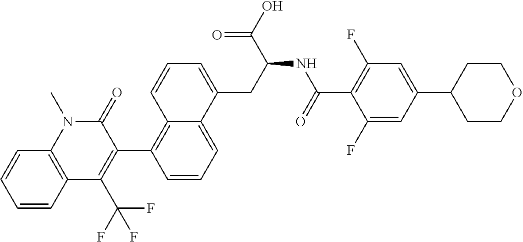 [SMILES: Cn1c(=O)c(-c2cccc3c(C[C@H](NC(=O)c4c(F)cc(C5CCOCC5)cc4F)C(=O)O)cccc23)c(C(F)(F)F)c2ccccc21] |
| 374* | 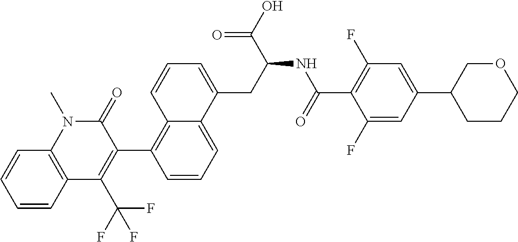 [SMILES: Cn1c(=O)c(-c2cccc3c(C[C@H](NC(=O)c4c(F)cc(C5CCCOC5)cc4F)C(=O)O)cccc23)c(C(F)(F)F)c2ccccc21] |
| 375** | 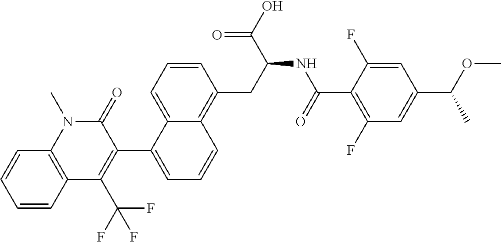 [SMILES: CO[C@H](C)c1cc(F)c(C(=O)N[C@@H](Cc2cccc3c(-c4c(C(F)(F)F)c5ccccc5n(C)c4=O)cccc23)C(=O)O)c(F)c1] |
| 376 | 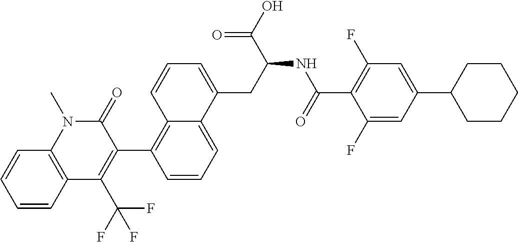 [SMILES: Cn1c(=O)c(-c2cccc3c(C[C@H](NC(=O)c4c(F)cc(C5CCCCC5)cc4F)C(=O)O)cccc23)c(C(F)(F)F)c2ccccc21] |
| 377 | 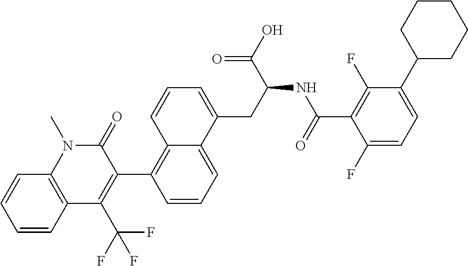 [SMILES: Cn1c(=O)c(-c2cccc3c(C[C@H](NC(=O)c4c(F)ccc(C5CCCCC5)c4F)C(=O)O)cccc23)c(C(F)(F)F)c2ccccc21] |
| 378 | 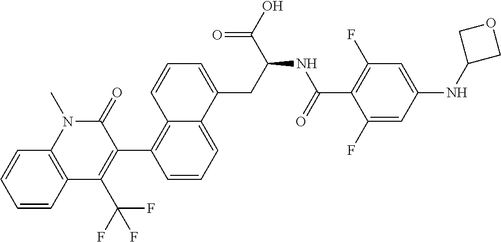 [SMILES: Cn1c(=O)c(-c2cccc3c(C[C@H](NC(=O)c4c(F)cc(NC5COC5)cc4F)C(=O)O)cccc23)c(C(F)(F)F)c2ccccc21] |
| 379 | 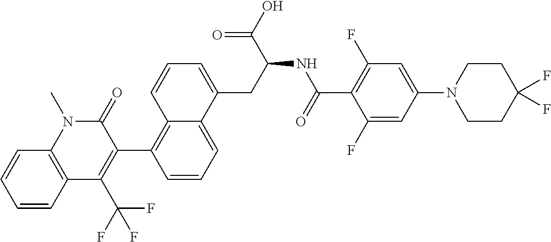 [SMILES: Cn1c(=O)c(-c2cccc3c(C[C@H](NC(=O)c4c(F)cc(N5CCC(F)(F)CC5)cc4F)C(=O)O)cccc23)c(C(F)(F)F)c2ccccc21] |
| 380 | 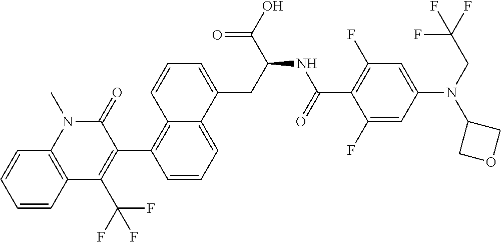 [SMILES: Cn1c(=O)c(-c2cccc3c(C[C@H](NC(=O)c4c(F)cc(N(CC(F)(F)F)C5COC5)cc4F)C(=O)O)cccc23)c(C(F)(F)F)c2ccccc21] |
| 381 | 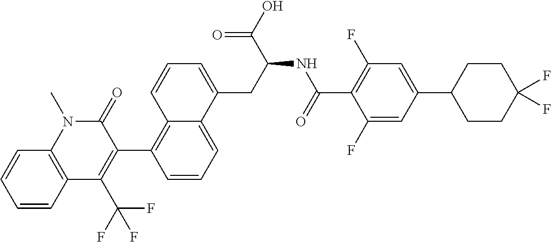 [SMILES: Cn1c(=O)c(-c2cccc3c(C[C@H](NC(=O)c4c(F)cc(C5CCC(F)(F)CC5)cc4F)C(=O)O)cccc23)c(C(F)(F)F)c2ccccc21] |
| 382 | 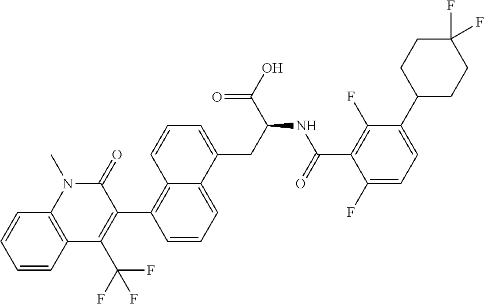 [SMILES: Cn1c(=O)c(-c2cccc3c(C[C@H](NC(=O)c4c(F)ccc(C5CCC(F)(F)CC5)c4F)C(=O)O)cccc23)c(C(F)(F)F)c2ccccc21] |
| 383 | 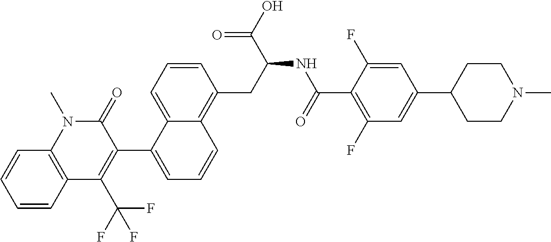 [SMILES: CN1CCC(c2cc(F)c(C(=O)N[C@@H](Cc3cccc4c(-c5c(C(F)(F)F)c6ccccc6n(C)c5=O)cccc34)C(=O)O)c(F)c2)CC1] |
| 384 | 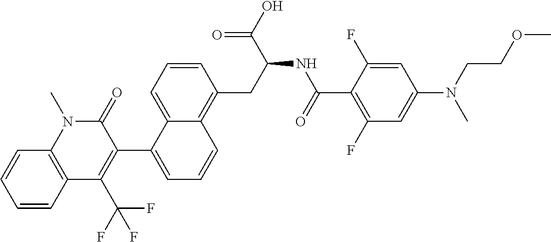 [SMILES: COCCN(C)c1cc(F)c(C(=O)N[C@@H](Cc2cccc3c(-c4c(C(F)(F)F)c5ccccc5n(C)c4=O)cccc23)C(=O)O)c(F)c1] |
| 385 | 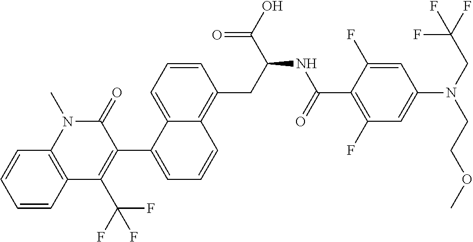 [SMILES: COCCN(CC(F)(F)F)c1cc(F)c(C(=O)N[C@@H](Cc2cccc3c(-c4c(C(F)(F)F)c5ccccc5n(C)c4=O)cccc23)C(=O)O)c(F)c1] |
| 386* | 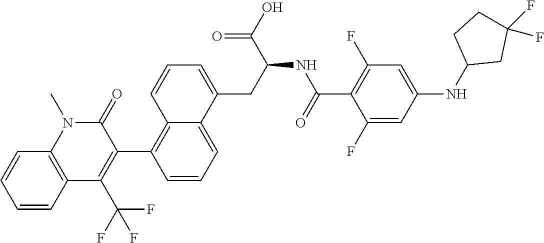 [SMILES: Cn1c(=O)c(-c2cccc3c(C[C@H](NC(=O)c4c(F)cc(NC5CCC(F)(F)C5)cc4F)C(=O)O)cccc23)c(C(F)(F)F)c2ccccc21] |
| 387 | 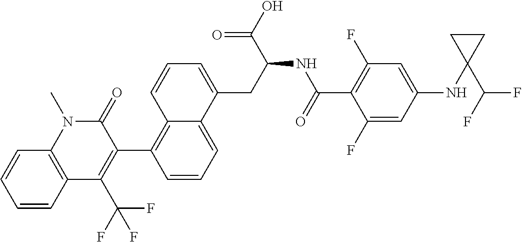 [SMILES: Cn1c(=O)c(-c2cccc3c(C[C@H](NC(=O)c4c(F)cc(NC5(C(F)F)CC5)cc4F)C(=O)O)cccc23)c(C(F)(F)F)c2ccccc21] |
| 388 | 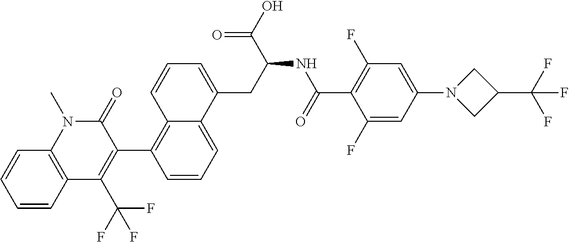 [SMILES: Cn1c(=O)c(-c2cccc3c(C[C@H](NC(=O)c4c(F)cc(N5CC(C(F)(F)F)C5)cc4F)C(=O)O)cccc23)c(C(F)(F)F)c2ccccc21] |
| 389 | 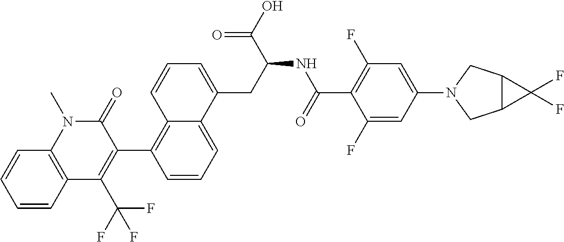 [SMILES: Cn1c(=O)c(-c2cccc3c(C[C@H](NC(=O)c4c(F)cc(N5CC6C(C5)C6(F)F)cc4F)C(=O)O)cccc23)c(C(F)(F)F)c2ccccc21] |
| 390 | 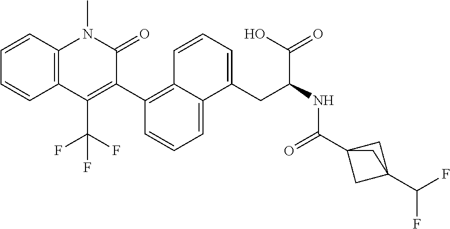 [SMILES: Cn1c(=O)c(-c2cccc3c(C[C@H](NC(=O)C45CC(C(F)F)(C4)C5)C(=O)O)cccc23)c(C(F)(F)F)c2ccccc21] |
| 391 | 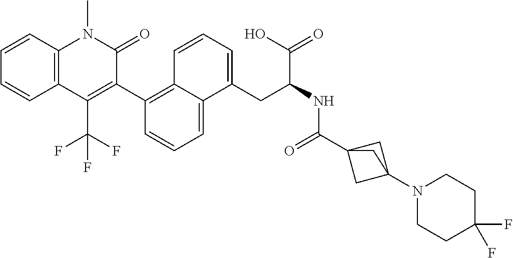 [SMILES: Cn1c(=O)c(-c2cccc3c(C[C@H](NC(=O)C45CC(N6CCC(F)(F)CC6)(C4)C5)C(=O)O)cccc23)c(C(F)(F)F)c2ccccc21] |
| 392 | 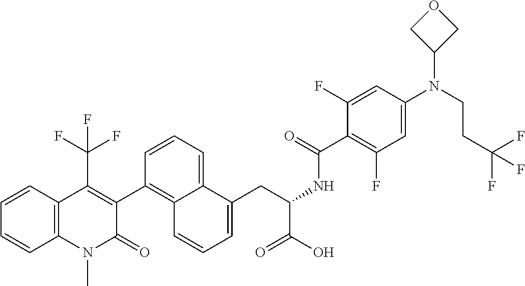 [SMILES: Cn1c(=O)c(-c2cccc3c(C[C@H](NC(=O)c4c(F)cc(N(CCC(F)(F)F)C5COC5)cc4F)C(=O)O)cccc23)c(C(F)(F)F)c2ccccc21] |
| 393 | 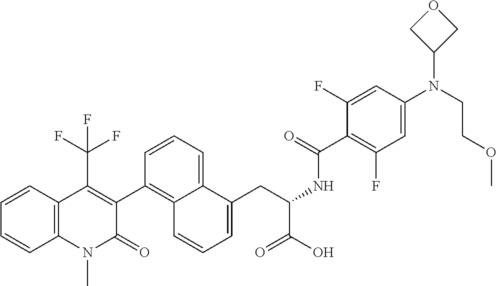 [SMILES: COCCN(c1cc(F)c(C(=O)N[C@@H](Cc2cccc3c(-c4c(C(F)(F)F)c5ccccc5n(C)c4=O)cccc23)C(=O)O)c(F)c1)C1COC1] |
| 394 | 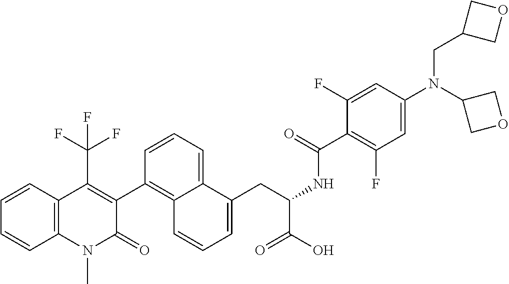 [SMILES: Cn1c(=O)c(-c2cccc3c(C[C@H](NC(=O)c4c(F)cc(N(CC5COC5)C5COC5)cc4F)C(=O)O)cccc23)c(C(F)(F)F)c2ccccc21] |
| 395 | 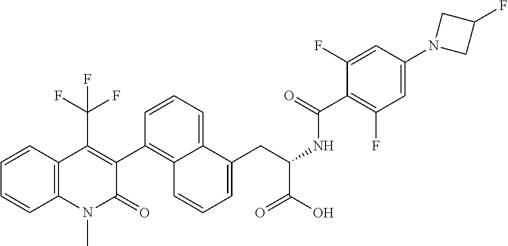 [SMILES: Cn1c(=O)c(-c2cccc3c(C[C@H](NC(=O)c4c(F)cc(N5CC(F)C5)cc4F)C(=O)O)cccc23)c(C(F)(F)F)c2ccccc21] |
| 396 | 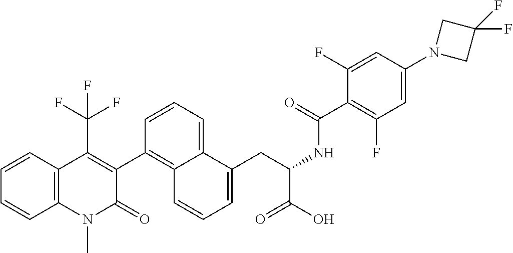 [SMILES: Cn1c(=O)c(-c2cccc3c(C[C@H](NC(=O)c4c(F)cc(N5CC(F)(F)C5)cc4F)C(=O)O)cccc23)c(C(F)(F)F)c2ccccc21] |
| 397 | 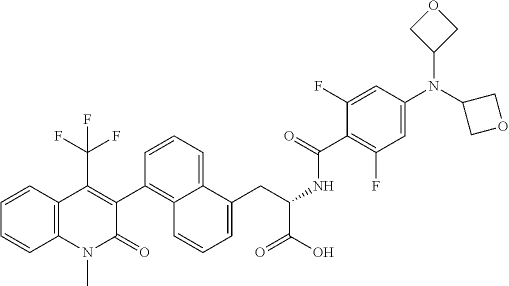 [SMILES: Cn1c(=O)c(-c2cccc3c(C[C@H](NC(=O)c4c(F)cc(N(C5COC5)C5COC5)cc4F)C(=O)O)cccc23)c(C(F)(F)F)c2ccccc21] |
| 398 | 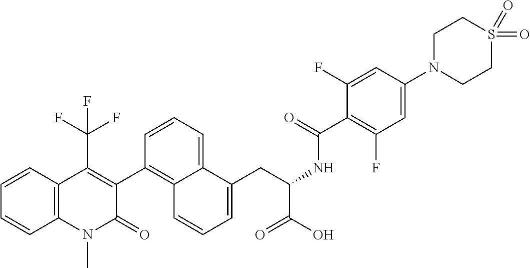 [SMILES: Cn1c(=O)c(-c2cccc3c(C[C@H](NC(=O)c4c(F)cc(N5CCS(=O)(=O)CC5)cc4F)C(=O)O)cccc23)c(C(F)(F)F)c2ccccc21] |
| 399** | 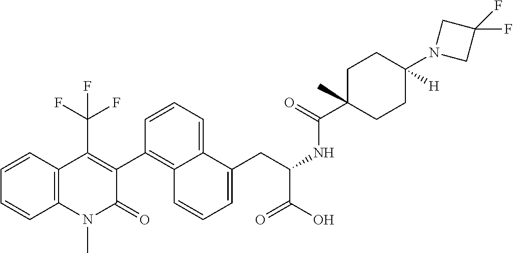 [SMILES: Cn1c(=O)c(-c2cccc3c(C[C@H](NC(=O)[C@]4(C)CC[C@@H](N5CC(F)(F)C5)CC4)C(=O)O)cccc23)c(C(F)(F)F)c2ccccc21] |
| 400** | 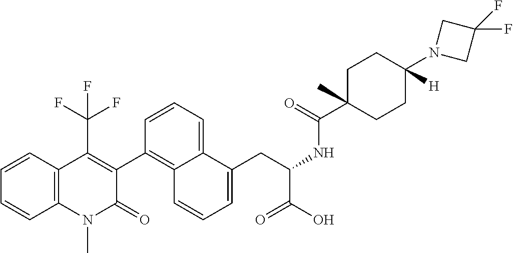 [SMILES: Cn1c(=O)c(-c2cccc3c(C[C@H](NC(=O)[C@]4(C)CC[C@H](N5CC(F)(F)C5)CC4)C(=O)O)cccc23)c(C(F)(F)F)c2ccccc21] |
| 401** | 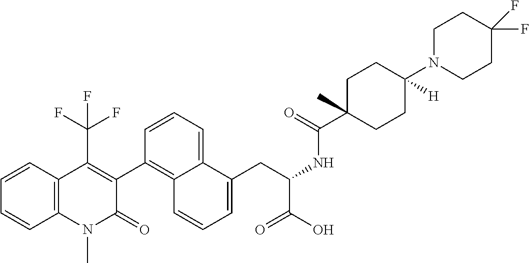 [SMILES: Cn1c(=O)c(-c2cccc3c(C[C@H](NC(=O)[C@]4(C)CC[C@@H](N5CCC(F)(F)CC5)CC4)C(=O)O)cccc23)c(C(F)(F)F)c2ccccc21] |
| 402** | 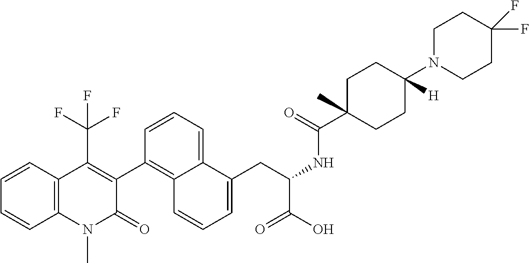 [SMILES: Cn1c(=O)c(-c2cccc3c(C[C@H](NC(=O)[C@]4(C)CC[C@H](N5CCC(F)(F)CC5)CC4)C(=O)O)cccc23)c(C(F)(F)F)c2ccccc21] |
| 403** | 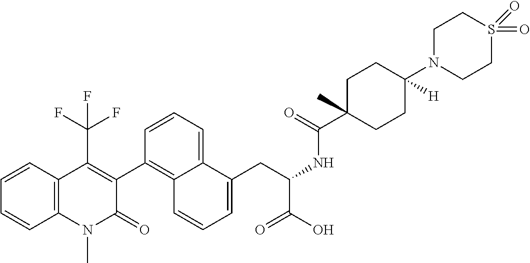 [SMILES: Cn1c(=O)c(-c2cccc3c(C[C@H](NC(=O)[C@]4(C)CC[C@@H](N5CCS(=O)(=O)CC5)CC4)C(=O)O)cccc23)c(C(F)(F)F)c2ccccc21] |
| 404** | 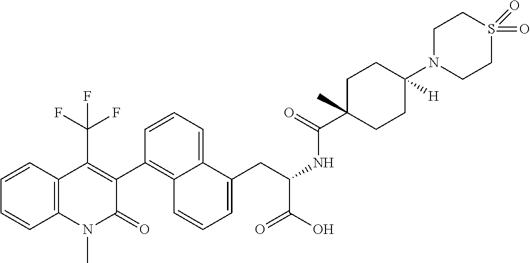 [SMILES: Cn1c(=O)c(-c2cccc3c(C[C@H](NC(=O)[C@]4(C)CC[C@@H](N5CCS(=O)(=O)CC5)CC4)C(=O)O)cccc23)c(C(F)(F)F)c2ccccc21] |
| 405** | 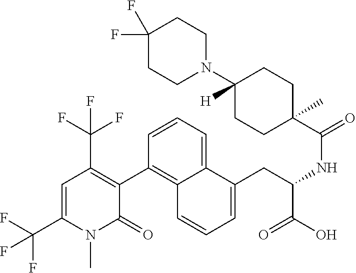 [SMILES: Cn1c(C(F)(F)F)cc(C(F)(F)F)c(-c2cccc3c(C[C@H](NC(=O)[C@]4(C)CC[C@@H](N5CCC(F)(F)CC5)CC4)C(=O)O)cccc23)c1=O] |
| 406** | 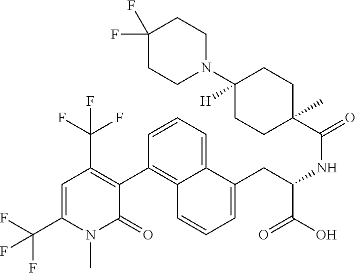 [SMILES: Cn1c(C(F)(F)F)cc(C(F)(F)F)c(-c2cccc3c(C[C@H](NC(=O)[C@]4(C)CC[C@H](N5CCC(F)(F)CC5)CC4)C(=O)O)cccc23)c1=O] |
| 407** | 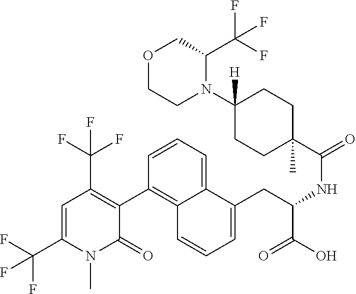 [SMILES: Cn1c(C(F)(F)F)cc(C(F)(F)F)c(-c2cccc3c(C[C@H](NC(=O)[C@]4(C)CC[C@@H](N5CCOC[C@@H]5C(F)(F)F)CC4)C(=O)O)cccc23)c1=O] |
| 408** | 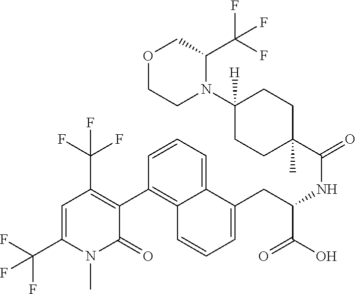 [SMILES: Cn1c(C(F)(F)F)cc(C(F)(F)F)c(-c2cccc3c(C[C@H](NC(=O)[C@]4(C)CC[C@H](N5CCOC[C@@H]5C(F)(F)F)CC4)C(=O)O)cccc23)c1=O] |
| 409** | 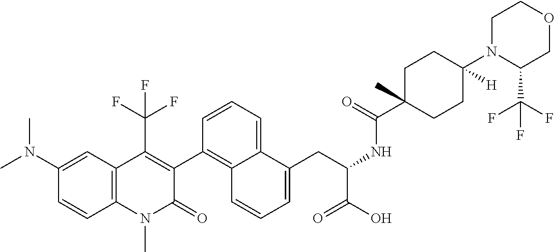 [SMILES: CN(C)c1ccc2c(c1)c(C(F)(F)F)c(-c1cccc3c(C[C@H](NC(=O)[C@]4(C)CC[C@@H](N5CCOC[C@@H]5C(F)(F)F)CC4)C(=O)O)cccc13)c(=O)n2C] |
| 410** | 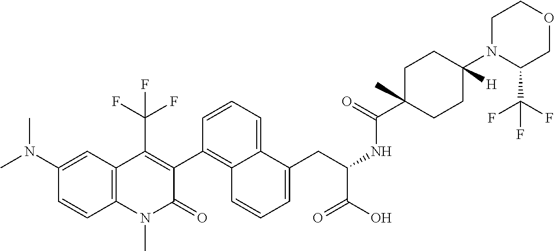 [SMILES: CN(C)c1ccc2c(c1)c(C(F)(F)F)c(-c1cccc3c(C[C@H](NC(=O)[C@]4(C)CC[C@H](N5CCOC[C@@H]5C(F)(F)F)CC4)C(=O)O)cccc13)c(=O)n2C] |
| 411** | 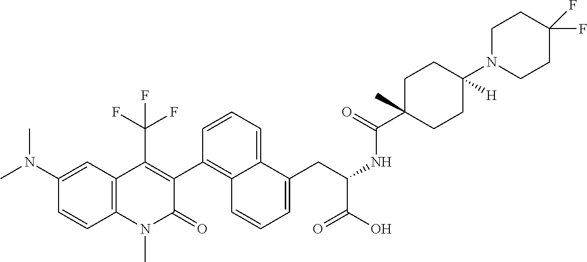 [SMILES: CN(C)c1ccc2c(c1)c(C(F)(F)F)c(-c1cccc3c(C[C@H](NC(=O)[C@]4(C)CC[C@@H](N5CCC(F)(F)CC5)CC4)C(=O)O)cccc13)c(=O)n2C] |
| 412** | 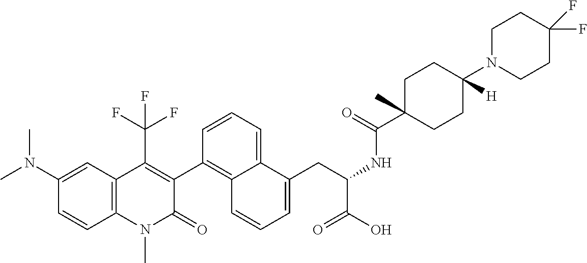 [SMILES: CN(C)c1ccc2c(c1)c(C(F)(F)F)c(-c1cccc3c(C[C@H](NC(=O)[C@]4(C)CC[C@H](N5CCC(F)(F)CC5)CC4)C(=O)O)cccc13)c(=O)n2C] |
| 413** | 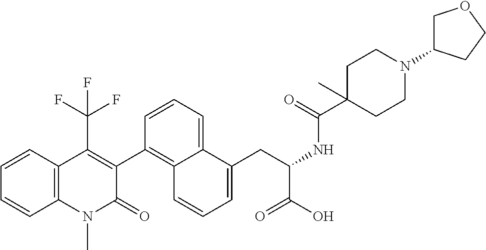 [SMILES: Cn1c(=O)c(-c2cccc3c(C[C@H](NC(=O)C4(C)CCN([C@H]5CCOC5)CC4)C(=O)O)cccc23)c(C(F)(F)F)c2ccccc21] |
| 414** | 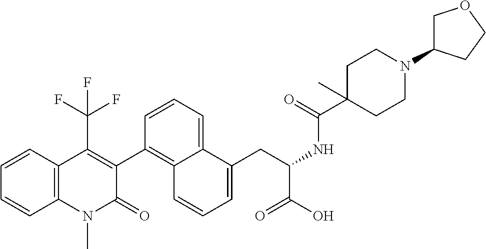 [SMILES: Cn1c(=O)c(-c2cccc3c(C[C@H](NC(=O)C4(C)CCN([C@@H]5CCOC5)CC4)C(=O)O)cccc23)c(C(F)(F)F)c2ccccc21] |
| 415** | 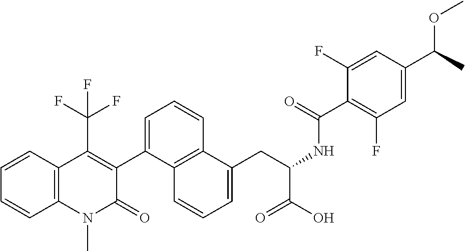 [SMILES: CO[C@@H](C)c1cc(F)c(C(=O)N[C@@H](Cc2cccc3c(-c4c(C(F)(F)F)c5ccccc5n(C)c4=O)cccc23)C(=O)O)c(F)c1] |
| 416** | 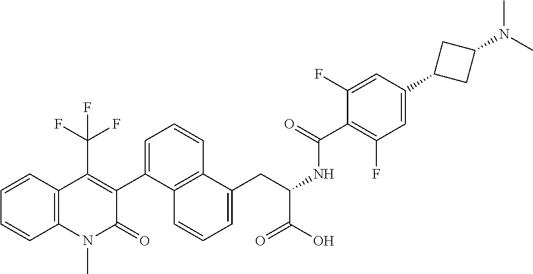 [SMILES: Cn1c(=O)c(-c2cccc3c(C[C@H](NC(=O)c4c(F)cc([C@H]5C[C@@H](N(C)C)C5)cc4F)C(=O)O)cccc23)c(C(F)(F)F)c2ccccc21] |
| 417** | 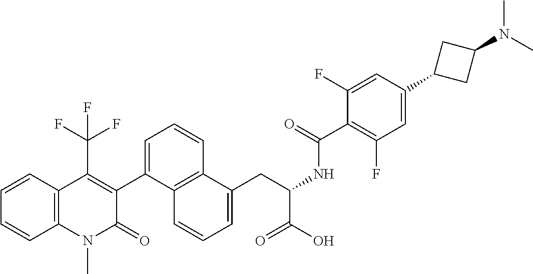 [SMILES: Cn1c(=O)c(-c2cccc3c(C[C@H](NC(=O)c4c(F)cc([C@H]5C[C@H](N(C)C)C5)cc4F)C(=O)O)cccc23)c(C(F)(F)F)c2ccccc21] |
| 418** | 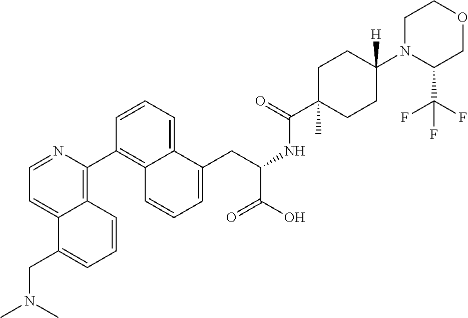 [SMILES: CN(C)Cc1cccc2c(-c3cccc4c(C[C@H](NC(=O)[C@]5(C)CC[C@@H](N6CCOC[C@@H]6C(F)(F)F)CC5)C(=O)O)cccc34)nccc12] |
| 419** | 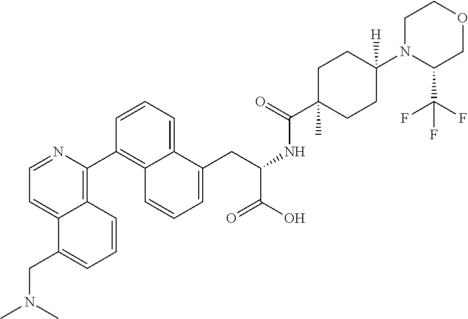 [SMILES: CN(C)Cc1cccc2c(-c3cccc4c(C[C@H](NC(=O)[C@]5(C)CC[C@H](N6CCOC[C@@H]6C(F)(F)F)CC5)C(=O)O)cccc34)nccc12] |
| 420 | 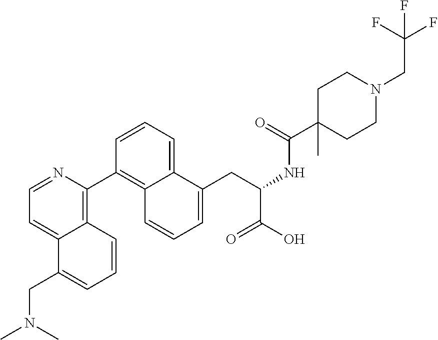 [SMILES: CN(C)Cc1cccc2c(-c3cccc4c(C[C@H](NC(=O)C5(C)CCN(CC(F)(F)F)CC5)C(=O)O)cccc34)nccc12] |
| 421 | 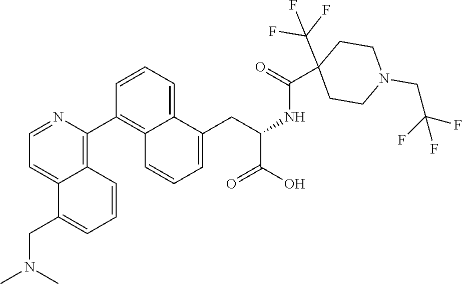 [SMILES: CN(C)Cc1cccc2c(-c3cccc4c(C[C@H](NC(=O)C5(C(F)(F)F)CCN(CC(F)(F)F)CC5)C(=O)O)cccc34)nccc12] |
| 422** | 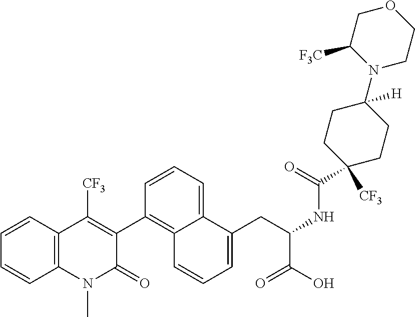 [SMILES: Cn1c(=O)c(-c2cccc3c(C[C@H](NC(=O)[C@]4(C(F)(F)F)CC[C@@H](N5CCOC[C@@H]5C(F)(F)F)CC4)C(=O)O)cccc23)c(C(F)(F)F)c2ccccc21] |
| 423** | 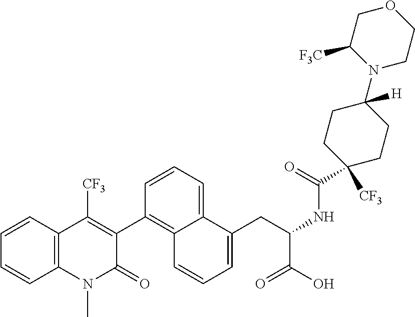 [SMILES: Cn1c(=O)c(-c2cccc3c(C[C@H](NC(=O)[C@]4(C(F)(F)F)CC[C@H](N5CCOC[C@@H]5C(F)(F)F)CC4)C(=O)O)cccc23)c(C(F)(F)F)c2ccccc21] |
| 424** | 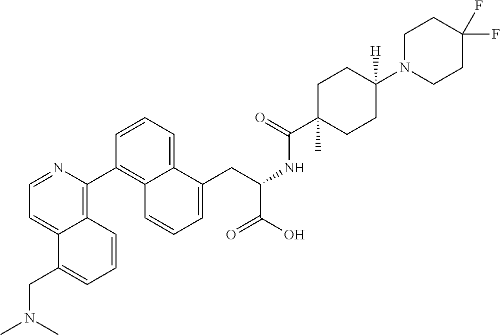 [SMILES: CN(C)Cc1cccc2c(-c3cccc4c(C[C@H](NC(=O)[C@]5(C)CC[C@H](N6CCC(F)(F)CC6)CC5)C(=O)O)cccc34)nccc12] |
| 425** | 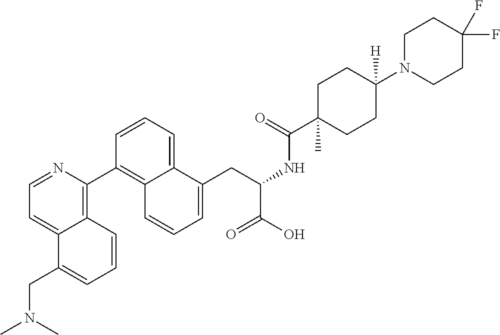 [SMILES: CN(C)Cc1cccc2c(-c3cccc4c(C[C@H](NC(=O)[C@]5(C)CC[C@H](N6CCC(F)(F)CC6)CC5)C(=O)O)cccc34)nccc12] |
| 426 | 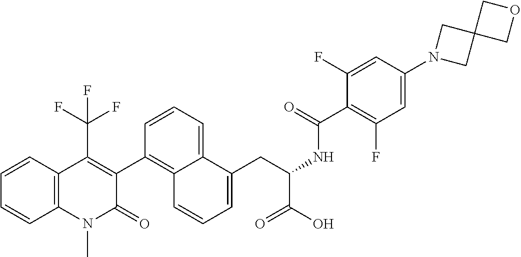 [SMILES: Cn1c(=O)c(-c2cccc3c(C[C@H](NC(=O)c4c(F)cc(N5CC6(COC6)C5)cc4F)C(=O)O)cccc23)c(C(F)(F)F)c2ccccc21] |
| 427 | 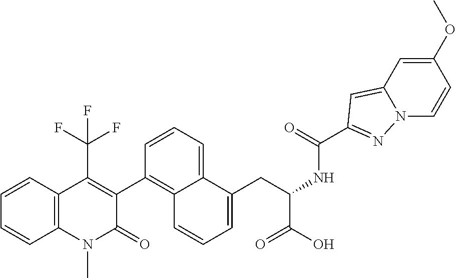 [SMILES: COc1ccn2nc(C(=O)N[C@@H](Cc3cccc4c(-c5c(C(F)(F)F)c6ccccc6n(C)c5=O)cccc34)C(=O)O)cc2c1] |
| 428 | 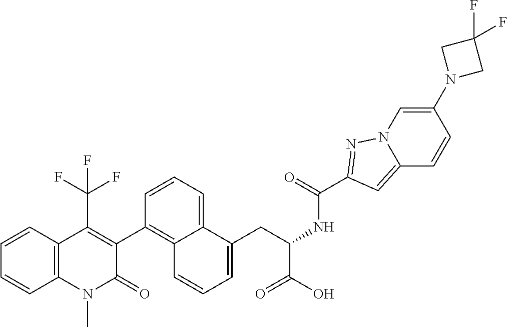 [SMILES: Cn1c(=O)c(-c2cccc3c(C[C@H](NC(=O)c4cc5ccc(N6CC(F)(F)C6)cn5n4)C(=O)O)cccc23)c(C(F)(F)F)c2ccccc21] |
| 429 | 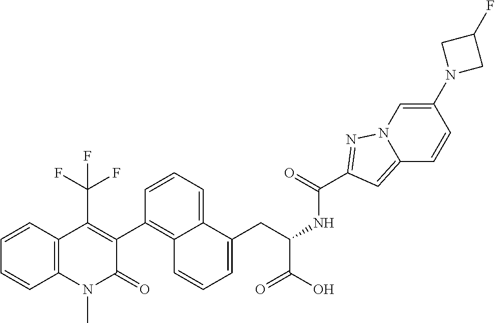 [SMILES: Cn1c(=O)c(-c2cccc3c(C[C@H](NC(=O)c4cc5ccc(N6CC(F)C6)cn5n4)C(=O)O)cccc23)c(C(F)(F)F)c2ccccc21] |
| 430 | 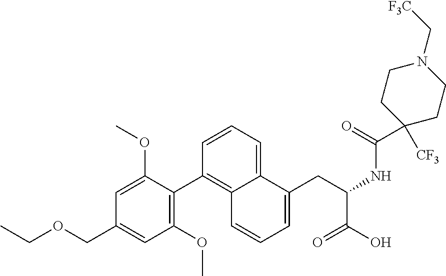 [SMILES: CCOCc1cc(OC)c(-c2cccc3c(C[C@H](NC(=O)C4(C(F)(F)F)CCN(CC(F)(F)F)CC4)C(=O)O)cccc23)c(OC)c1] |
| 431 | 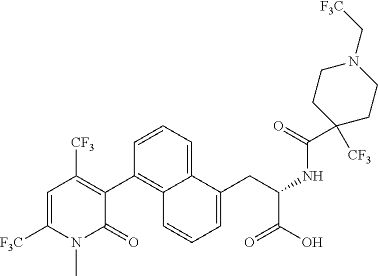 [SMILES: Cn1c(C(F)(F)F)cc(C(F)(F)F)c(-c2cccc3c(C[C@H](NC(=O)C4(C(F)(F)F)CCN(CC(F)(F)F)CC4)C(=O)O)cccc23)c1=O] |
| 432 | 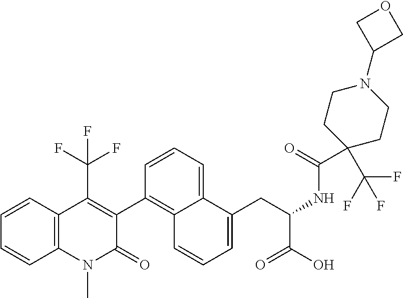 [SMILES: Cn1c(=O)c(-c2cccc3c(C[C@H](NC(=O)C4(C(F)(F)F)CCN(C5COC5)CC4)C(=O)O)cccc23)c(C(F)(F)F)c2ccccc21] |
| 433 | 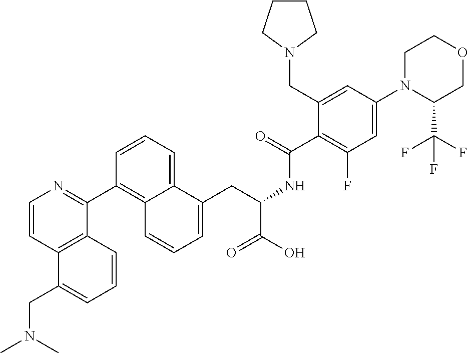 [SMILES: CN(C)Cc1cccc2c(-c3cccc4c(C[C@H](NC(=O)c5c(F)cc(N6CCOC[C@@H]6C(F)(F)F)cc5CN5CCCC5)C(=O)O)cccc34)nccc12] |
| 434 | 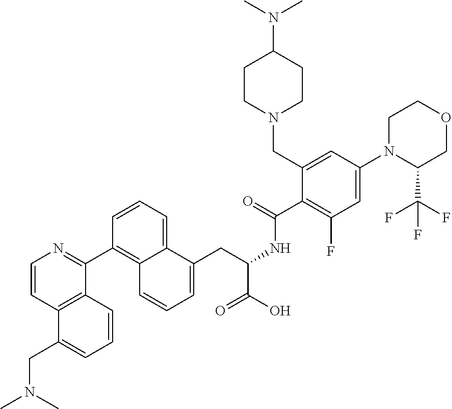 [SMILES: CN(C)Cc1cccc2c(-c3cccc4c(C[C@H](NC(=O)c5c(F)cc(N6CCOC[C@@H]6C(F)(F)F)cc5CN5CCC(N(C)C)CC5)C(=O)O)cccc34)nccc12] |
| 435 | 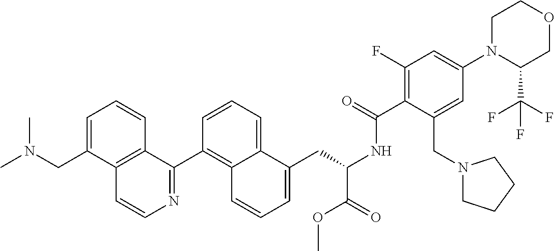 [SMILES: COC(=O)[C@H](Cc1cccc2c(-c3nccc4c(CN(C)C)cccc34)cccc12)NC(=O)c1c(F)cc(N2CCOC[C@@H]2C(F)(F)F)cc1CN1CCCC1] |
| 436 | 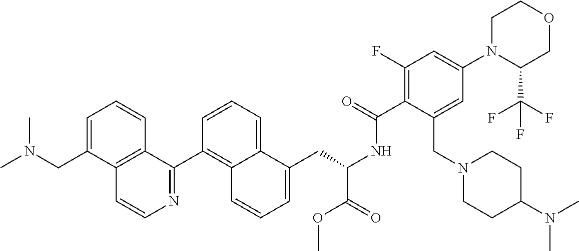 |
| 437 | 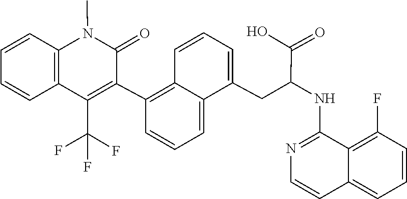 [SMILES: Cn1c(=O)c(-c2cccc3c(CC(Nc4nccc5cccc(F)c45)C(=O)O)cccc23)c(C(F)(F)F)c2ccccc21] |
| 438 | 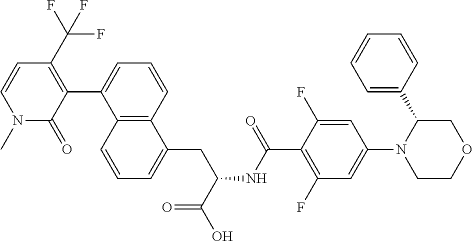 [SMILES: Cn1ccc(C(F)(F)F)c(-c2cccc3c(C[C@H](NC(=O)c4c(F)cc(N5CCOC[C@H]5c5ccccc5)cc4F)C(=O)O)cccc23)c1=O] |
| 439 | 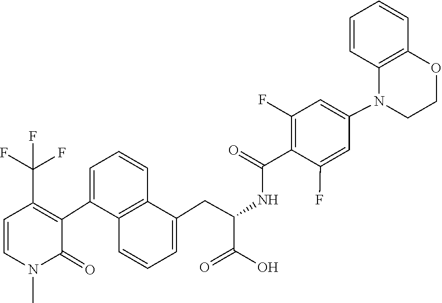 [SMILES: Cn1ccc(C(F)(F)F)c(-c2cccc3c(C[C@H](NC(=O)c4c(F)cc(N5CCOc6ccccc65)cc4F)C(=O)O)cccc23)c1=O] |
| 440 | 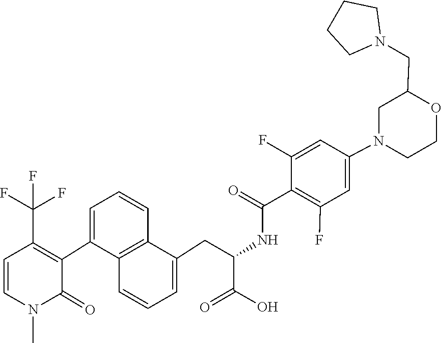 [SMILES: Cn1ccc(C(F)(F)F)c(-c2cccc3c(C[C@H](NC(=O)c4c(F)cc(N5CCOC(CN6CCCC6)C5)cc4F)C(=O)O)cccc23)c1=O] |
| 441 | 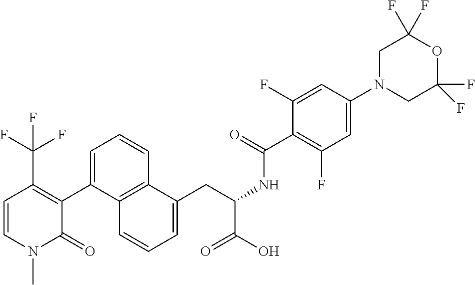 [SMILES: Cn1ccc(C(F)(F)F)c(-c2cccc3c(C[C@H](NC(=O)c4c(F)cc(N5CC(F)(F)OC(F)(F)C5)cc4F)C(=O)O)cccc23)c1=O] |
| 442 | 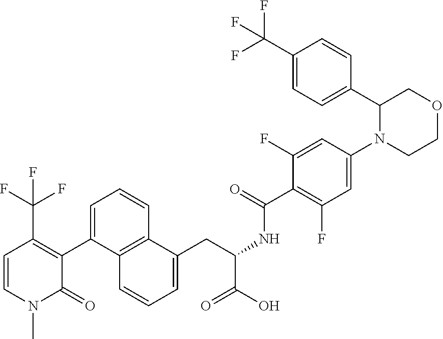 [SMILES: Cn1ccc(C(F)(F)F)c(-c2cccc3c(C[C@H](NC(=O)c4c(F)cc(N5CCOCC5c5ccc(C(F)(F)F)cc5)cc4F)C(=O)O)cccc23)c1=O] |
| 443 | 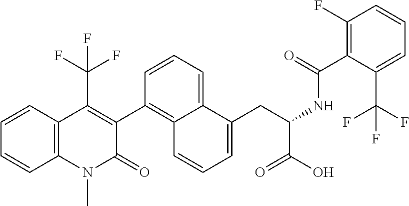 [SMILES: Cn1c(=O)c(-c2cccc3c(C[C@H](NC(=O)c4c(F)cccc4C(F)(F)F)C(=O)O)cccc23)c(C(F)(F)F)c2ccccc21] |
| 444 | 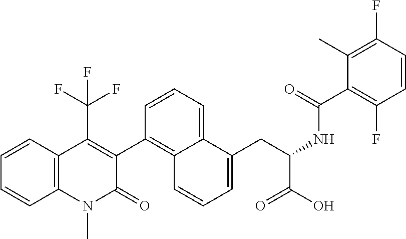 [SMILES: Cc1c(F)ccc(F)c1C(=O)N[C@@H](Cc1cccc2c(-c3c(C(F)(F)F)c4ccccc4n(C)c3=O)cccc12)C(=O)O] |
| 445 | 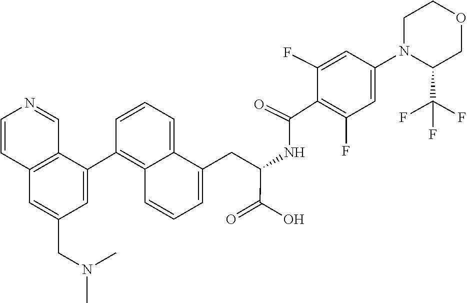 [SMILES: CN(C)Cc1cc(-c2cccc3c(C[C@H](NC(=O)c4c(F)cc(N5CCOC[C@@H]5C(F)(F)F)cc4F)C(=O)O)cccc23)c2cnccc2c1] |
| 446 | 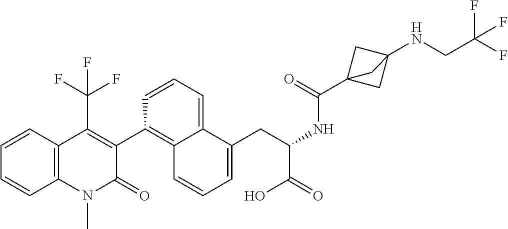 |
| 447 | 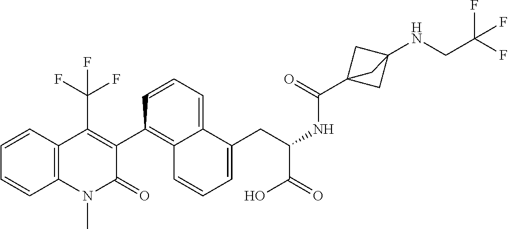 |
| 448 | 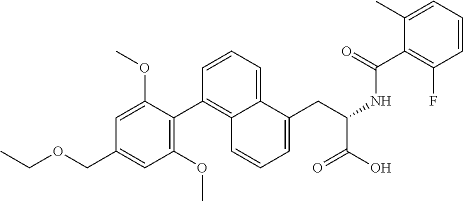 [SMILES: CCOCc1cc(OC)c(-c2cccc3c(C[C@H](NC(=O)c4c(C)cccc4F)C(=O)O)cccc23)c(OC)c1] |
| 449 | 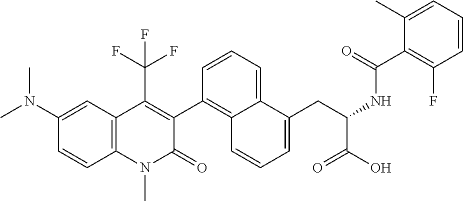 [SMILES: Cc1cccc(F)c1C(=O)N[C@@H](Cc1cccc2c(-c3c(C(F)(F)F)c4cc(N(C)C)ccc4n(C)c3=O)cccc12)C(=O)O] |
| 450 | 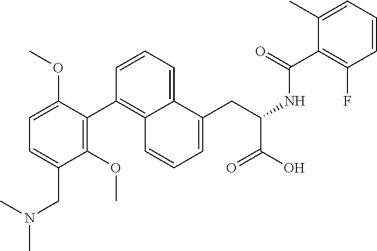 [SMILES: COc1ccc(CN(C)C)c(OC)c1-c1cccc2c(C[C@H](NC(=O)c3c(C)cccc3F)C(=O)O)cccc12] |
| 451 | 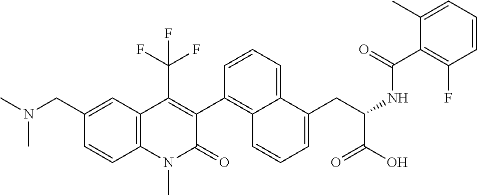 [SMILES: Cc1cccc(F)c1C(=O)N[C@@H](Cc1cccc2c(-c3c(C(F)(F)F)c4cc(CN(C)C)ccc4n(C)c3=O)cccc12)C(=O)O] |
| 452 | 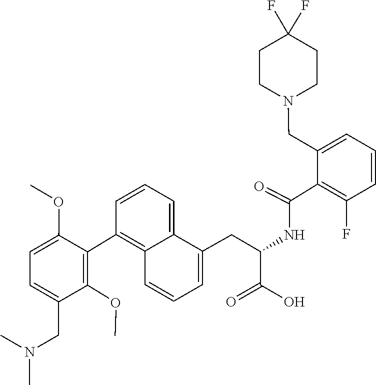 [SMILES: COc1ccc(CN(C)C)c(OC)c1-c1cccc2c(C[C@H](NC(=O)c3c(F)cccc3CN3CCC(F)(F)CC3)C(=O)O)cccc12] |
| 453 | 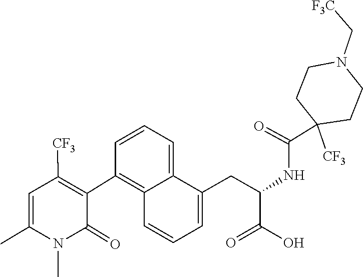 [SMILES: Cc1cc(C(F)(F)F)c(-c2cccc3c(C[C@H](NC(=O)C4(C(F)(F)F)CCN(CC(F)(F)F)CC4)C(=O)O)cccc23)c(=O)n1C] |
| 454 | 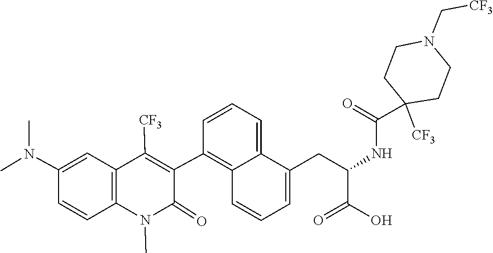 [SMILES: CN(C)c1ccc2c(c1)c(C(F)(F)F)c(-c1cccc3c(C[C@H](NC(=O)C4(C(F)(F)F)CCN(CC(F)(F)F)CC4)C(=O)O)cccc13)c(=O)n2C] |
| 455 | 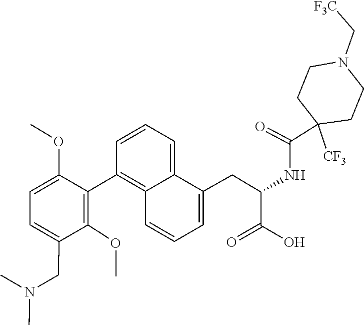 [SMILES: COc1ccc(CN(C)C)c(OC)c1-c1cccc2c(C[C@H](NC(=O)C3(C(F)(F)F)CCN(CC(F)(F)F)CC3)C(=O)O)cccc12] |
| 456 | 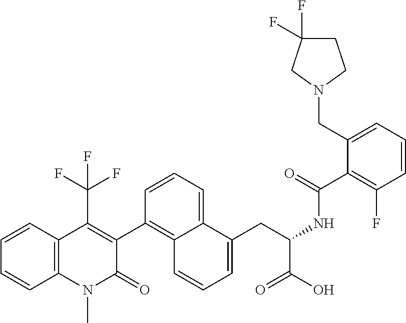 [SMILES: Cn1c(=O)c(-c2cccc3c(C[C@H](NC(=O)c4c(F)cccc4CN4CCC(F)(F)C4)C(=O)O)cccc23)c(C(F)(F)F)c2ccccc21] |
| 457** | 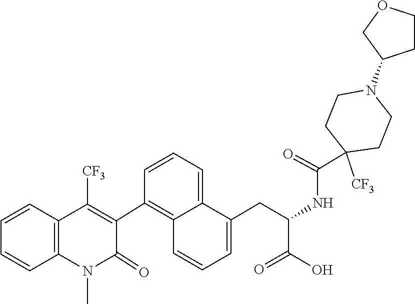 [SMILES: Cn1c(=O)c(-c2cccc3c(C[C@H](NC(=O)C4(C(F)(F)F)CCN([C@H]5CCOC5)CC4)C(=O)O)cccc23)c(C(F)(F)F)c2ccccc21] |
| 458** | 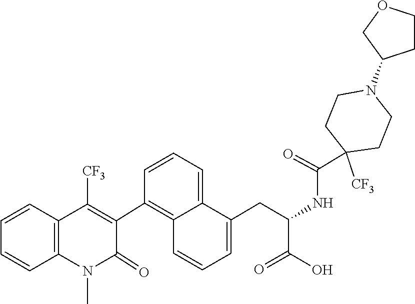 [SMILES: Cn1c(=O)c(-c2cccc3c(C[C@H](NC(=O)C4(C(F)(F)F)CCN([C@H]5CCOC5)CC4)C(=O)O)cccc23)c(C(F)(F)F)c2ccccc21] |
| 459 | 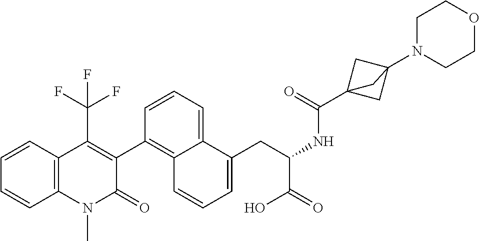 [SMILES: Cn1c(=O)c(-c2cccc3c(C[C@H](NC(=O)C45CC(N6CCOCC6)(C4)C5)C(=O)O)cccc23)c(C(F)(F)F)c2ccccc21] |
| 460 | 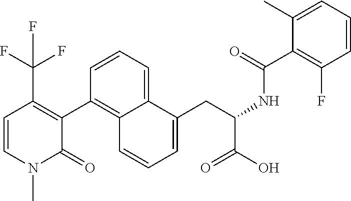 [SMILES: Cc1cccc(F)c1C(=O)N[C@@H](Cc1cccc2c(-c3c(C(F)(F)F)ccn(C)c3=O)cccc12)C(=O)O] |
| 461 | 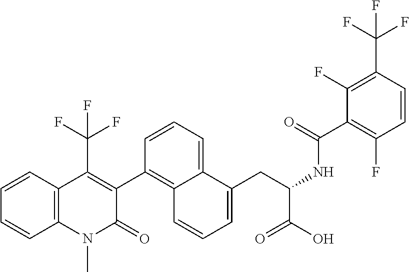 [SMILES: Cn1c(=O)c(-c2cccc3c(C[C@H](NC(=O)c4c(F)ccc(C(F)(F)F)c4F)C(=O)O)cccc23)c(C(F)(F)F)c2ccccc21] |
| 462* | 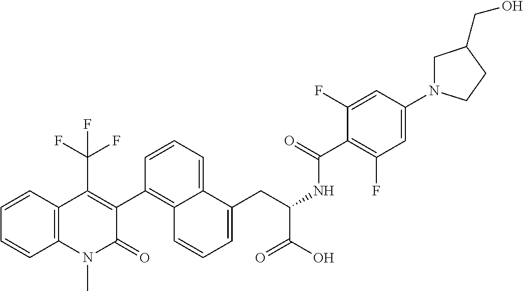 [SMILES: Cn1c(=O)c(-c2cccc3c(C[C@H](NC(=O)c4c(F)cc(N5CCC(CO)C5)cc4F)C(=O)O)cccc23)c(C(F)(F)F)c2ccccc21] |
| 463* | 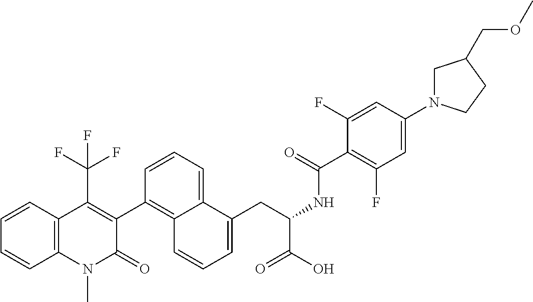 [SMILES: COCC1CCN(c2cc(F)c(C(=O)N[C@@H](Cc3cccc4c(-c5c(C(F)(F)F)c6ccccc6n(C)c5=O)cccc34)C(=O)O)c(F)c2)C1] |
| 464** | 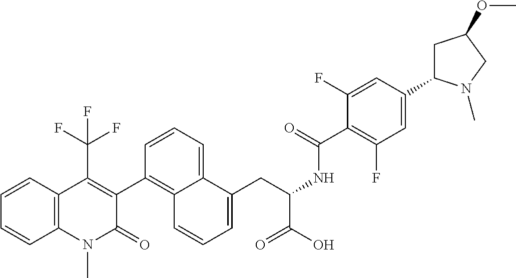 [SMILES: CO[C@@H]1C[C@@H](c2cc(F)c(C(=O)N[C@@H](Cc3cccc4c(-c5c(C(F)(F)F)c6ccccc6n(C)c5=O)cccc34)C(=O)O)c(F)c2)N(C)C1] |
| 465** | 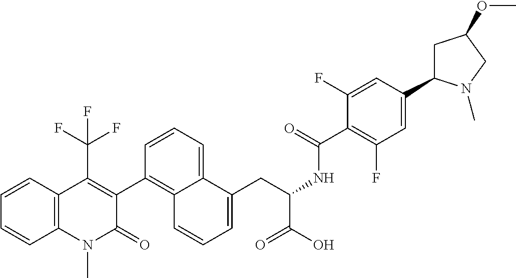 [SMILES: CO[C@@H]1C[C@H](c2cc(F)c(C(=O)N[C@@H](Cc3cccc4c(-c5c(C(F)(F)F)c6ccccc6n(C)c5=O)cccc34)C(=O)O)c(F)c2)N(C)C1] |
| 466** | 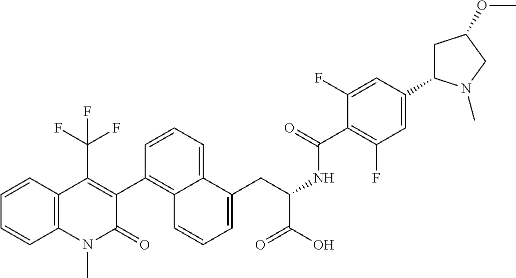 [SMILES: CO[C@H]1C[C@@H](c2cc(F)c(C(=O)N[C@@H](Cc3cccc4c(-c5c(C(F)(F)F)c6ccccc6n(C)c5=O)cccc34)C(=O)O)c(F)c2)N(C)C1] |
| 467** | 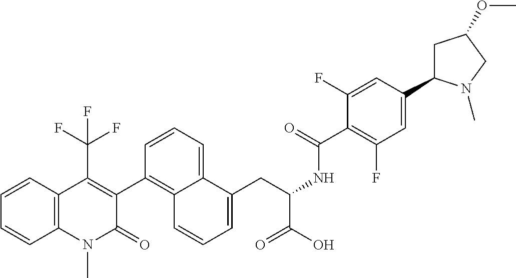 [SMILES: CO[C@H]1C[C@H](c2cc(F)c(C(=O)N[C@@H](Cc3cccc4c(-c5c(C(F)(F)F)c6ccccc6n(C)c5=O)cccc34)C(=O)O)c(F)c2)N(C)C1] |
| 468 | 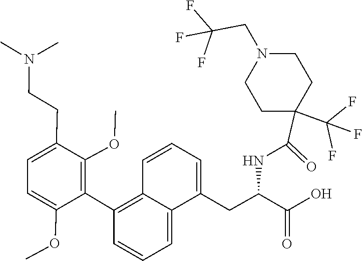 [SMILES: COc1ccc(CCN(C)C)c(OC)c1-c1cccc2c(C[C@H](NC(=O)C3(C(F)(F)F)CCN(CC(F)(F)F)CC3)C(=O)O)cccc12] |
| 469 | 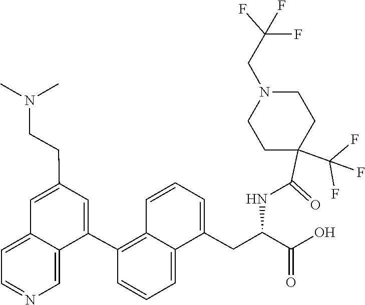 [SMILES: CN(C)CCc1cc(-c2cccc3c(C[C@H](NC(=O)C4(C(F)(F)F)CCN(CC(F)(F)F)CC4)C(=O)O)cccc23)c2cnccc2c1] |

*Denotes a racemic compound;
**Denotes a single isomer with unknown absolute stereochemistry.

In some aspects, the compound is any of the compounds represented in Table 1, Table 2, and Table 3, or a pharmaceutically acceptable salt or solvate thereof. In some embodiments, the compound is any one of the compounds represented in Table 1, Table 2, Table 3, and Table 4, or a pharmaceutically acceptable salt or solvate thereof. In some embodiments, the compound is any of the compounds represented in Table 1, or a pharmaceutically acceptable salt or solvate thereof. In some embodiments, the compound is any of the compounds represented in Table 2, or a pharmaceutically acceptable salt or solvate thereof. In some embodiments, the compound is any of the compounds represented in Table 3, or a pharmaceutically acceptable salt or solvate thereof. In some embodiments, the compound is any one of the compounds represented in Table 4, or a pharmaceutically acceptable salt or solvate thereof.

In some aspects, the compound or salt of Formula (I), (I-a), (II), or (III) is selected from a compound or salt of Table 1, Table 2, Table 3, or Table 4. In some aspects, the compound or salt of Formula (I), (I-a), (II), or (III) is selected from a compound or salt of Table 1. In some aspects, the compound or salt of Formula (I), (I-a). (II), or (III) is selected from a compound or salt of Table 2. In some aspects, the compound or salt of Formula (I), (I-a), (II), or (III) is selected from a compound or salt of Table 3. In some aspects, the compound or salt of Formula (I), (I-a), (II), or (III) is selected from a compound or salt of Table 4

In some aspects, the compound or salt of Formula (I), (I-a), (II), or (III) is selected from a compound of Table 1, Table 2, Table 3, or Table 4. In some aspects, the compound or salt of Formula (I), (I-a), (II), or (III) is selected from a compound of Table 1. In some aspects, the compound or salt of Formula (I), (I-a), (II), or (III) is selected from a compound of Table 2. In some aspects, the compound or salt of Formula (I), (I-a), (II), or (III) is selected from a compound of Table 3. In some aspects, the compound or salt of Formula (I), (I-a), (II), or (III) is selected from a compound of Table 4.

In some aspects, the compound or salt of Formula (I), (I-a), (II), or (III) is selected from Table 1, Table 2, Table 3, Table 4, Table 14, Table 15, Table 16, Table 17, Table 18, Table 19, Table 20, Table 21, Table 22, Table 23, Table 24, Table 25, Table 26, Table 27, or Table 28. In some aspects, the compound or salt of Formula (I), (I-a), (II), or (III) is selected from Table 14, Table 15, Table 16, Table 17, Table 18, Table 19, Table 20, Table 21, Table 22, Table 23, Table 24, Table 25, Table 26, Table 27, or Table 28.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formulas (I), (I-a), (II) or (III), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and(S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formulas (I), (I-a), (II), or (III), herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formulas (I), (I-a), (II), or (III), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113 (3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formulas (I), (I-a), (II), or (III), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

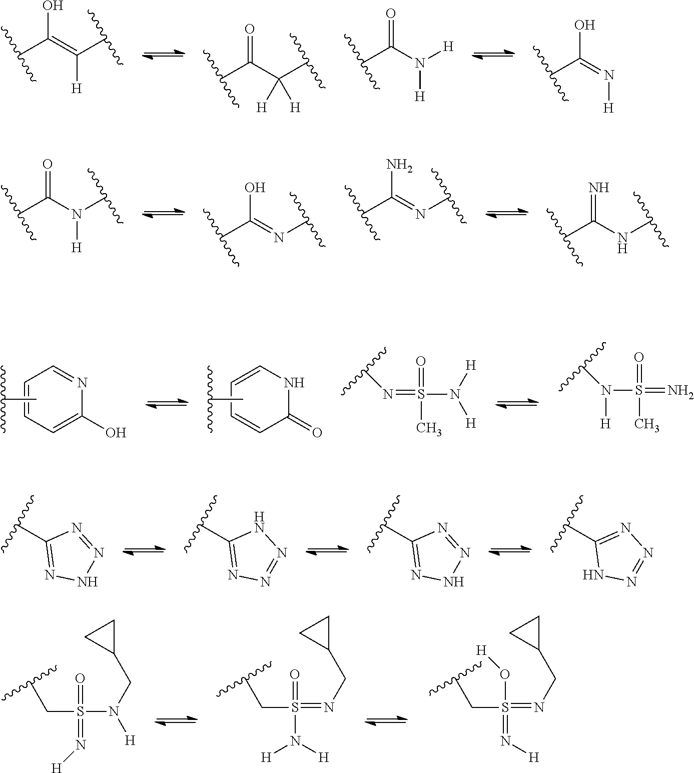

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the 1H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon 14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formulas (I), (I-a), (II), or (III). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formulas (I), (I-a), (II), or (III), include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formulas (I), (I-a), (II), or (III), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formulas (I), (I-a), (II), or (III). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of (I), (I-a), (II), or (III) may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In some embodiments, the design of a prodrug increases the lipophilicity of the pharmaceutical agent. In some embodiments, the design of a prodrug increases the effective water solubility. See, e.g., Fedorak et al., Am. J. Physiol., 269: G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formulas (I), (I-a), (II), or (III) and at least one pharmaceutically acceptable excipient.

Pharmaceutical compositions can be formulated using one or more physiologically acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically acceptable salt form.

Methods for formulation of the conjugates can include formulating any of the compounds, salts or conjugates with one or more inert, pharmaceutically acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. Alternatively, the compounds, salts or conjugates can be lyophilized or in powder form for re-constitution with a suitable vehicle, e.g., sterile pyrogen-free water, before use.

Pharmaceutical compositions can comprise at least one active ingredient (e.g., a compound, salt or conjugate). The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nanoparticles and nanocapsules) or in macroemulsions.

Pharmaceutical compositions as often further can comprise more than one active compound (e.g., a compound, salt or conjugate and other agents) as necessary for the particular indication being treated. The active compounds can have complementary activities that do not adversely affect each other. For example, the composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The compositions and formulations can be sterilized. Sterilization can be accomplished by filtration through sterile filtration.

The compositions can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions can be lyophilized or in powder form for reconstitution with a suitable vehicle, e.g., sterile pyrogen-free water, before use.

For parenteral administration, the compounds, salts or conjugates can be formulated in a unit dosage injectable form (e.g., solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Pharmaceutical formulations can be prepared for storage by mixing a compound, salt or conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

A compound or salt of any one of Formulas (I), (I-a), (II), or (III) may be formulated in any suitable pharmaceutical formulation. A pharmaceutical formulation of the present disclosure typically contains an active ingredient (e.g., compound or salt of any one of Formulas (I), (I-a), (II), or (III), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, sub cutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of compound or salt of any one of Formulas (I) and (II) will be dependent on the mammal being treated, the severity of the disorder or condition, the rate of administration, the disposition of the compound or salt of any one of Formulas (I) and (II) and the discretion of the prescribing physician.

In some embodiments, the disclosure provides a pharmaceutical composition for oral administration containing at least one compound or salt of any one of Formulas (I) and (II) and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formulas (I) and (II) moistened with an inert liquid diluent.

In some embodiments, the disclosure provides a pharmaceutical composition for injection containing a compound or salt of any one of Formulas (I) and (II) disclosed herein and a pharmaceutical excipient suitable for injection. Components and amounts of agents in the composition are as described herein.

In certain embodiments, the compound or salt of any one of Formulas (I) and (II) may be formulated for injection as aqueous or oil suspensions, emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Pharmaceutical compositions may also be prepared from a compound or salt of any one of Formulas (I) and (II) and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson. Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990, Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

In some embodiments, a method of treating a subject in need thereof may comprise administering to the subject a therapeutically effective amount of a compound of the present disclosure. As used herein, the term “therapeutically effective amount” means the amount of an inhibitor that is sufficient to reduce the expression and/or activity of α4β7 integrin in a subject or in a cell.

A compound of the present disclosure may be administered to the subject using various different administration routes, including oral, rectal, transmucosal, topical, transdermal, inhalation, intravenous, subcutaneous, intradermal, intramuscular, intra-articular, intrathecal, intraventricular, intravenous, intraperitoneal, intranasal, or intraocular routes of administration.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some embodiments, the present disclosure provides a method of modulating α4β7 integrin in a subject in need thereof, comprising administering to the subject a compound of Formulas (I), (I-a), (II), or (III) or pharmaceutically acceptable salt thereof. In some embodiments, the present disclosure provides a method of modulating α4β7 integrin in a subject in need thereof, comprising administering to the subject a pharmaceutical composition comprising a compound of Formulas (I), (I-a), (II), or (III) or a pharmaceutically acceptable salt thereof, and an excipient.

In some embodiments, the present disclosure provides a method of treating an disease or condition comprising administering to a subject in need thereof a compound of Formulas (I), (I-a), (II), or (III) or pharmaceutically acceptable salt thereof. In some embodiments, the present disclosure provides a method of treating a disease or condition comprising administering to a subject in need thereof a pharmaceutical composition comprising a compound of Formulas (I), (I-a), (II), or (III) or a pharmaceutically acceptable salt thereof, and an excipient. In some embodiments, the disease or condition is an inflammatory disease or condition.

In some embodiments, the disease or condition is selected from: inflammatory bowel disease, ulcerative colitis, Crohn's disease, graft-versus-host disease, type 1 diabetes, immune-mediated colitis, checkpoint inhibitor induced colitis, and primary sclerosing cholangitis. In some embodiments, the disease or condition is selected from: inflammatory bowel disease, ulcerative colitis, Crohn's disease, graft-versus-host disease, type 1 diabetes, and primary sclerosing cholangitis. In some embodiments, the disease or condition is selected from: inflammatory bowel disease, ulcerative colitis, Crohn's disease, graft-versus-host disease, immune-mediated colitis, checkpoint inhibitor induced colitis, and primary sclerosing cholangitis. In some embodiments, the disease or condition is selected from: inflammatory bowel disease, ulcerative colitis, Crohn's disease, graft-versus-host disease, and primary sclerosing cholangitis. In some embodiments, the disease or condition is selected from: inflammatory bowel disease, ulcerative colitis, and Crohn's disease. In some embodiments, the disease or condition is inflammatory bowel disease. In some embodiments, the disease or condition is ulcerative colitis. In some embodiments, the disease or condition is Crohn's disease. In some embodiments, the disease or condition is graft-versus-host disease. In some embodiments, the disease or condition is type 1 diabetes. In some embodiments, the disease or condition is immune-mediated colitis. checkpoint inhibitor induced colitis. In some embodiments, the disease or condition is primary sclerosing cholangitis.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1 to 70 show general and exemplary procedures for the preparation of the claimed compounds of Formulas (I), (I-a), (II), and (III). One of ordinary skill in the art will appreciate that variations on the following synthetic procedures may be necessary to obtain compounds of Formulas (I), (I-a), (II), and (III), including changes in protecting group chemistry, reaction conditions, and/or order of synthetic steps. Such variations are within the ability of the ordinarily skilled artisan.

### Example 1: Synthesis of Compound 1

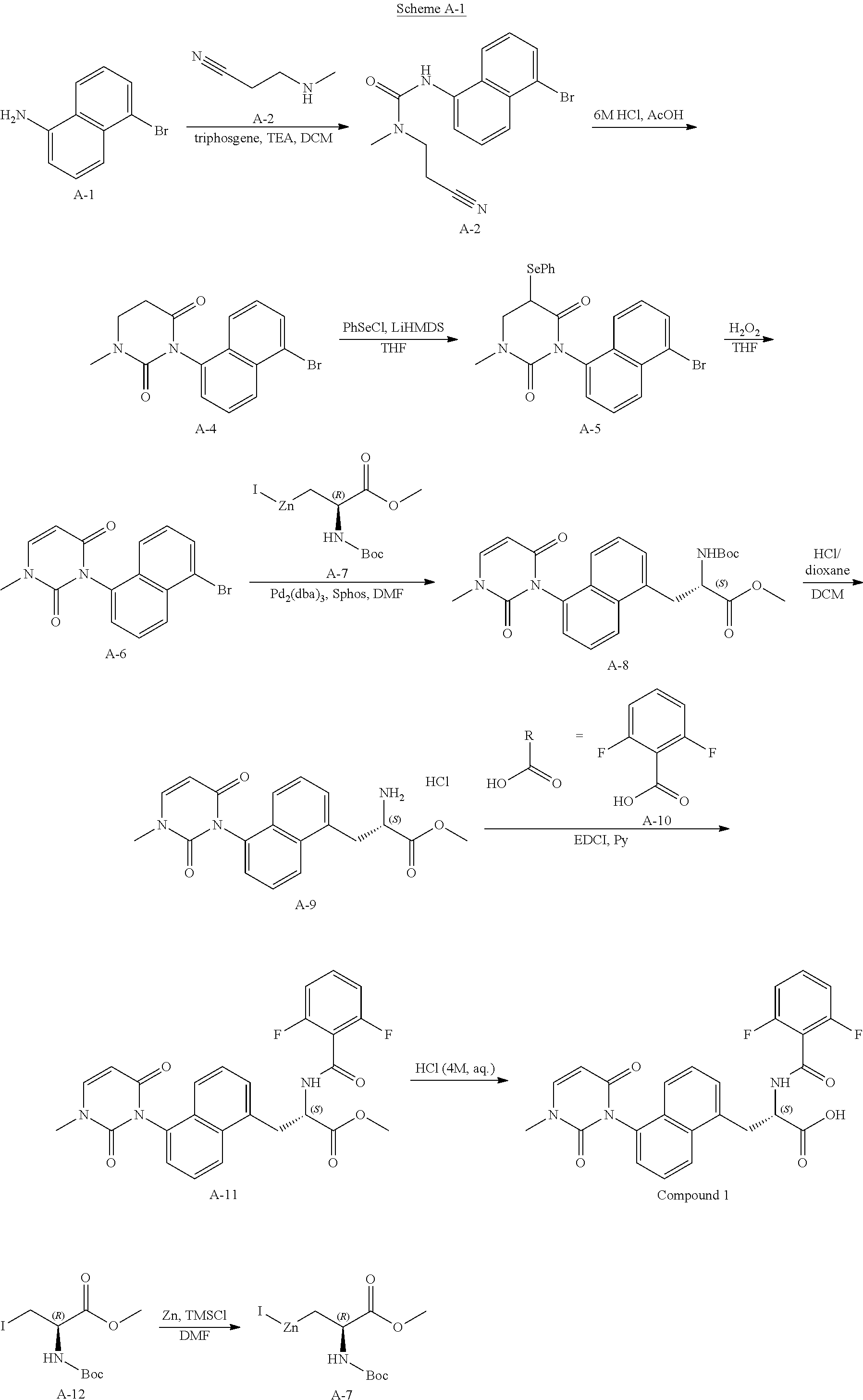

Step 1: To a solution of compound A-1 (10.0 g, 45.0 mmol, 1.00 eq) and TEA (9.11 g, 90.1 mmol, 12.5 mL, 2.00 eq) in DCM (480 mL) was added a solution of triphosgene (4.68 g, 15.8 mmol, 0.350 eq) in DCM (20 mL) at 0° C., and stirred at 0° C. for 2 hr. Compound A-2 (7.58 g, 90.1 mmol, 2.00 eq) was then added at 0° C. The mixture was stirred at 20° C. for 2 hrs. The reaction mixture was filtered and concentrated under reduced pressure to give a residue. The crude product was triturated with ethyl acetate (100 mL) at 25° C. for 30 min. Compound A-3 (13.0 g, crude) was obtained as white solid. LC-MS (M+H)⁺ 332.1. ¹H NMR (400 MHz, CDCl₃) δ 8.13 (d, J=8.8 Hz, 1H), 7.84-7.79 (m, 2H), 7.70 (d, J=7.2 Hz, 1H), 7.59-7.55 (m, 1H), 7.37-7.33 (m, 1H), 6.70 (s, 1H), 3.68 (t, J=6.4 Hz, 2H), 3.27 (s, 3H), 2.69 (t, J=6.4 Hz, 2H).

Step 2: To a solution of compound A-3 (3.00 g, 9.03 mmol, 1.00 eq) in HCl (6 M, 45.0 mL, 29.9 eq) and AcOH (15 mL) were stirred at 100° C. for 2 hrs. The reaction mixture was filtered and concentrated under reduced pressure. Compound A-4 (2.60 g, 7.80 mmol, 86.4% yield) was obtained as off-white solid. LC-MS (M+H)⁻ 332.9. ¹H NMR (400 MHz, CDCl₃) δ 8.35 (d, J=8.4 Hz, 1H), 7.80 (d, J=7.6 Hz, 1H), 7.70-7.64 (m, 1H), 7.57 (d, J=8.4 Hz, 1H), 7.41 (d, J=6.8 Hz, 1H), 7.34 (t, J=8.4 Hz, 1H), 3.66 (t, J=6.8 Hz, 2H), 3.15 (s, 3H), 3.11-2.97 (m, 2H).

Step 3: A solution of compound A-4 (1.00 g, 3.00 mmol, 1.00 eq) in THF (10 mL) was added LiHMDS (1 M, 6.00 mL, 2.00 eq) at −78° C. and stirred at −78° C. for 1 hr under N₂. A solution of PhSeCl (862 mg, 4.50 mmol, 1.50 eq) in THF (4.00 mL) was then added at −78° C. The mixture was stirred at 0° C. for 2 hr. The reaction mixture was added dropwise to HCl (6M, 50 mL) for quenching at 0° C., and then diluted with ethyl acetate 100 mL and extracted with ethyl acetate (100 mL*3). The combined organic layers were dried over Na₂SO₄, filtered and concentrated under reduced pressure to give a residue. The residue was purified by column chromatography (SiO₂, Petroleum ether:Ethyl acetate=1:0 to 0:1, Petroleum ether:Ethyl acetate=1:1, R_{f}=0.43, R_{f}=0.30). Compound A-5 (1.40 g, crude) was obtained as yellow solid. LC-MS: (M+H)⁺: 488.8.

Step 4: To a solution of compound A-5 (1.40 g, 2.87 mmol, 1.00 eq) in THF (30 mL) was added H₂O₂ (2.60 g, 22.9 mmol, 2.20 mL, 30% purity, 8.00 eq) at 0° C., and stirred at 20° C. for 0.5 hr. The reaction mixture was quenched by addition aq. Na₂SO₃ (sat., 50 mL) at 0° C., and then diluted with DCM (20 mL) and extracted with DCM (40 mL*3). The combined organic layers were dried over Na₂SO₄, filtered and concentrated under reduced pressure to give a residue. The crude product was triturated with petroleum ether:ethyl acetate=3:1 (10 mL) at 25° C. for 10 min. Compound A-6 (700 mg, 2.11 mmol, 73.7% yield) was obtained as off-white solid. LC-MS: (M+H)⁺: 332.9. ¹H NMR (400 MHz, CDCl₃) δ 8.40 (d, J=8.4 Hz, 1H), 7.82 (d, J=7.6 Hz, 1H), 7.71 (t, J=7.6 Hz, 1H), 7.55 (d, J=8.4 Hz, 1H), 7.46 (d, J=7.2 Hz, 1H), 7.35-7.31 (m, 2H), 5.94 (d, J=7.6 Hz, 1H), 3.48 (s, 3H).

Intermediate A-7: Zn (1.99 g, 30.4 mmol, 2.00 eq) was added to a three necked flask, and heated at 110° C. under vacuum for 10 min, then cooled to 25° C. To the flask was added a solution of TMSCl (990 mg, 9.11 mmol, 1.16 mL, 0.600 eq) in DMF (15 mL) and stirred at 25° C. for 20 min. And the supernatant liquor was removed by syringe. Then a mixture of compound A-12 (5.00 g, 15.2 mmol, 1.00 eq) and TMSCl (495 mg, 4.56 mmol, 578 μL, 0.300 eq) in DMF (20 mL) was added to the precipitate. And the resulting mixture was stirred at 25-45° C. for 1 hr. Compound A-7 (5.99 g in DMF (20 mL) was obtained as black solution. The solution of the reaction was used to next step.

Step 5: To a solution of compound A-6 (400 mg, 1.21 mmol, 1.00 eq), Pd, (dba); (111 mg, 121 μmol, 0.100 eq), SPhos (49.6 mg, 121 μmol, 0.100 eq) in DMF (10 mL) was added compound A-7 (1.50 g, 3.80 mmol, 3.15 eq) (1.50 g, in DMF (5 mL)) under N₂, and stirred at 85° C. for 2 hrs. The reaction mixture was diluted with H₂O 20 mL and extracted with ethyl acetate (50 mL*3). The combined organic layers were washed with water (100 mL*2), dried over Na₂SO₄, filtered and concentrated under reduced pressure to give a residue. Compound A-8 (500 mg, crude) was obtained as yellow solid. LC-MS (M-99)⁺ 354.0.

Step 6: To a solution of compound A-8 (0.500 g, 1.10 mmol, 1.00 eq) in DCM (2.00 mL) was added HCl/dioxane (4 M, 2.00 mL, 7.26 eq), and stirred at 20° C. for 1 hr. The reaction mixture was concentrated under reduced pressure to give a residue. The crude product was triturated with DCM (10 mL) at 20° C. for 10 min. Compound A-9 (140 mg, 359 μmol, 32.6% yield, HCl) was obtained as yellow solid. LC-MS (M+H)⁺ 354.2.

Step 7: To a solution of compound A-9 (120 mg, 308 μmol, 1.00 eq, HCl), compound A-10 (97.3 mg, 616 μmol, 2.00 eq) in pyridine (2.00 mL) was added EDCI (88.5 mg, 462 μmol, 1.50 eq), and stirred at 20° C. for 0.5 hr. The reaction mixture was concentrated under reduced pressure to give a residue. The residue was purified by prep-TLC (SiO₂, Petroleum ether:Ethyl acetate=0:1). Compound A-11 (80.0 mg, 162 μmol, 52.7% yield) was obtained as white solid. LC-MS (M+H)⁺ 494.2.

Step 8: To a solution of compound A-11 (70.0 mg, 142 μmol, 1.00 eq) in aqueous HCl (4 M, 2.00 mL, 56.4 eq) was stirred at 60° C. for 2 hrs. The reaction mixture was filtered and concentrated under reduced pressure to give a residue. The residue was purified by Prep-HPLC (column: Welch Xtimate C18 150*25 mm*5 um; mobile phase. [water (0.05% HCl)-ACN]; B %: 18%-48%, 10 min). Compound 1 (21.56 mg, 44.5 μmol, 31.4% yield, 98.9% purity) was obtained as white. LC-MS (M+H)⁻ 480.1. ¹H NMR (400 MHz, DMSO-d₆) δ 12.98 (s, 1H), 9.25 (d, J=7.6 Hz, 1H), 8.23 (d, J=8.8 Hz, 1H), 7.89 (d, J=8.0 Hz, 1H), 7.70 (t, J=8.0 Hz, 1H), 7.53-7.40 (m, 5H), 7.13 (t, J=8.0 Hz, 2H), 5.86-5.83 (m, 1H), 4.76-4.70 (m, 1H), 3.76-3.72 (m, 1H), 3.41-3.39 (m, 1H), 3.36 (d, J=2.8 Hz, 3H).

### Example 2: Synthesis of Compound 68

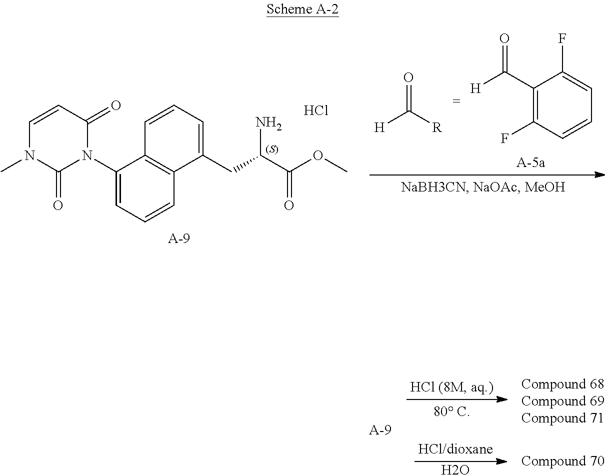

Step 1: To a solution compound A-9 (200 mg, 513 μmol, 1.00 eq, HCl) in MeOH (4.00 mL) was added NaOAc (21.0 mg, 257 μmol, 0.500 eq) at 25° C. stirred for 0.5 hr. To the mixture was then added compound A-5a (87.5 mg, 616 μmol, 66.3 uL, 1.20 eq) and mixture was kept at 25° C. for 0.5 hr. To the mixture was then added NaBH₃CN (38.7 mg, 616 μmol, 1.20 eq) and the mixture was stirred at 25° C. for 2 hrs. The reaction mixture was quenched by addition H₂O (10 mL) at 25° C., and then diluted with EtOAc (5 mL) and extracted with EtOAc 30 mL (10 mL*3). The combined organic layers were washed with brine 30 mL (10 mL*3), dried over Na₂SO₄, filtered and concentrated under reduced pressure to give a residue. Compound A-9a (200 mg, 417 μmol, 81.3% yield) was obtained as a yellow oil. LC-MS (M+H)⁺ 480.2. ¹H NMR (400 MHz, DMSO-d₆) δ 8.05 (dd, J₁=8.8 Hz, J₂=2.4 Hz, 1H), 7.89 (d, J=8.0 Hz, 1H), 7.62-7.55 (m, 1H), 7.44-7.29 (m, 5H), 7.06-7.00 (m, 2H), 5.85 (d, J=8.0 Hz, 1H), 3.80-3.72 (m, 2H), 3.62-3.54 (m, 1H), 3.42 (s, 3H), 3.36 (s, 3H), 3.35 (s, 1H), 2.58 (s, 1H).

Step 2: To a solution of compound A-9a (200 mg, 417 μmol, 1.00 eq) in aqueous HCl (8.00 M, 521 μL, 10.0 eq) the mixture was stirred 80° C. for 5 hrs. The reaction mixture was concentrated under reduced pressure to give a residue. The residue was purified by Prep-HPLC (column: Phenomenex Luna C18 200*40 mm*10 um; mobile phase: [water (FA)-ACN]; B %: 5%-35%, 10 min). Compound 68 (53.94 mg, 108 μmol, 26.0% yield, 93.9% purity) was obtained as a white solid. LC-MS (M+H)⁺ 466.2. ¹H NMR (400 MHz, DMSO-d₆+D₂O) δ 8.10-8.07 (m, 1H) 7.82 (d, J=8.0 Hz, 1H), 7.58 (t, J=7.6 Hz, 1H), 7.42-7.36 (m, 4H), 7.33-7.30 (m, 1H), 6.99 (t, J=8.0 Hz, 2H), 5.84-5.81 (m, 1H), 3.85-3.82 (m, 1H), 3.74 (s, 1H), 3.55-3.52 (m, 1H), 3.45-3.40 (m, 1H), 3.34-3.33 (m, 3H), 3.31-3.26 (m, 1H).

### Example 3: Synthesis of Compound 21

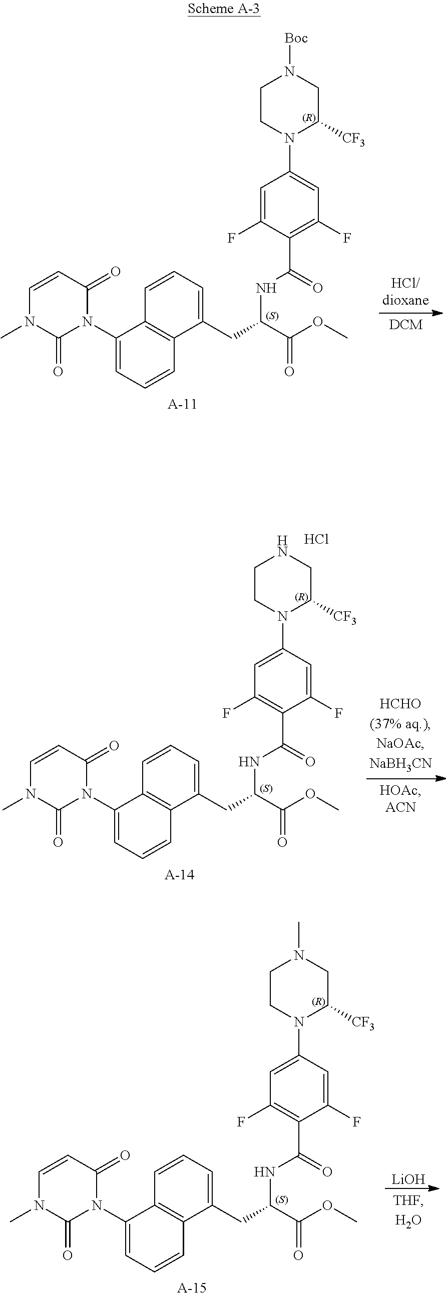

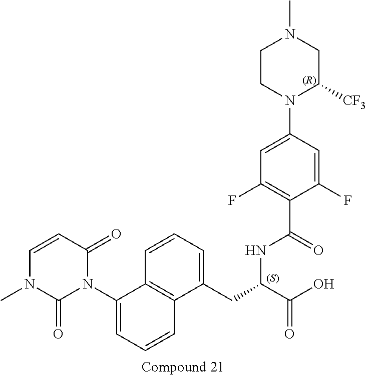

Step 1: To a solution of compound A-11 (113 mg, 152 μmol, 1.00 eq) in DCM (3.00 mL) was added HCl/dioxane (4.00 M, 37.9 uL, 1.00 eq) at 0° C., and the mixture was stirred at 20-25° C. for 1 hr. The reaction mixture was concentrated under reduced pressure to remove solvent to give a residue. The crude product was used into the next step without further purification. Compound A-14 (100 mg, 147 μmol, 96.8% yield, HCl) was obtained as a white solid.

Step 2: To a solution of compound A-14 (100 mg, 146 μmol, 1.00 eq, HCl), HCHO (47.6 mg, 586 μmol, 43.7 uL, 37.0% purity, 4.00 eq) and NaOAc (36.1 mg, 439 μmol, 3.00 eq) in AcOH (0.250 mL, 10.0% purity) and ACN (0.250 mL) was added NaBH₃CN (36.7 mg, 586 μmol, 4.00 eq) at 0° C. under N₂, stirred at 0° C. for 0.5 hr. Then the mixture was stirred under N₂ at 20-25° C. for 1.5 hrs. The residue was adjusted pH to 8 with sat. aq. NaHCO₃ and extracted with ethyl acetate (4.00 mL*2). The combined organic layers were washed with brine (4.00 mL*2), dried over Na₂SO₄ filtered and concentrated under reduced pressure to give a residue. The crude product was used into the next step without further purification. Compound A-15 (117 mg, crude) was obtained as a yellow gum. LC-MS (M+H)⁺: 660.5.

Step 3: To a solution of compound A-15 (117 mg, 177 μmol, 1.00 eq) in THF (5 mL) and H₂O (0.800 mL) was added LiOH. H₂O (29.8 mg, 709 μmol, 4.00 eq), the mixture was stirred at 20-25° C. for 1 hr. The reaction mixture was adjusted pH=5˜6 by addition of formic acid (98%˜ 100%) and concentrated under reduced pressure to remove solvent to give a residue. The residue was purified by prep-HPLC (FA condition; column: Phenomenex Luna C18 150*25 mm*10 um; mobile phase: [water (FA)-ACN]; B %: 2%-32%, 10 min). Compound 21 (47.4 mg, 72.8 μmol, 41.0% yield, 99.2% purity) was obtained as a white solid. ¹H NMR (400 MHz, DMSO-d₆) δ 8.90 (d, J=7.2 Hz, 1H), 8.24 (d, J=8.4 Hz, 1H), 7.88 (d, J=7.6 Hz, 1H), 7.69 (t, J=7.6 Hz, 1H), 7.36-7.55 (m, 4H), 6.75 (br d, J=11.6 Hz, 2H), 5.84 (dd, J₁=4.4 Hz, J₂=7.6, 1H), 4.94-5.07 (m, 1H), 4.66 (d, J=8.00 Hz, 1H), 3.71 (dd, J₁=4.00 Hz, J₂=14.0 Hz, 2H), 3.50 (d, J=12.0 Hz, 3H), 3.17-3.25 (m, 2H), 3.08 (d, J=12.0 Hz, 1H), 2.84 (brd, J=10.0 Hz, 1H), 2.19 (s, 4H), 1.89-2.03 (m, 1H). LC-MS (M+H)⁺ 646.4.

### Example 4: Synthesis of Compound 61

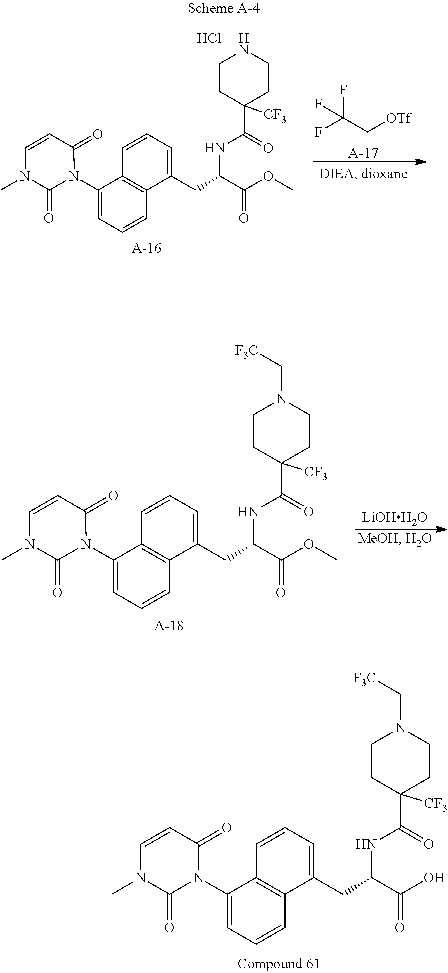

Step 1: To a solution of compound A-16 (70.0 mg, 131 μmol, 1.00 eq), compound A-17 (61.0 mg, 263 μmol, 2.00 eq) in dioxane (1.00 mL) was added DIEA (51.0 mg, 394 μmol, 68.7 uL, 3.00 eq). The mixture was stirred at 80° C. for 3 hrs. The reaction mixture was concentrated under reduced pressure to give a residue. Compound A-18 (80.0 mg, 130 μmol, 99.0% yield) was obtained as a white solid. LC-MS (M+H)⁺ 615.1.

Step 2: To a solution of compound A-18 (70.0 mg, 114 μmol, 1.00 eq) in MeOH (0.500 mL) was added LiOH·H₂O (7.17 mg, 171 μmol, 1.50 eq) in H₂O (0.50 mL). The mixture was stirred at 20° C. for 1 hr. The reaction mixture was concentrated under reduced pressure to give a residue. The residue was purified by prep-HPLC (column: Waters xbridge 150*25 mm 10 um; mobile phase: [water (NH₄HCO₃)-ACN]; B %: 12%-42%, 8 min). Compound 61 (17.76 mg, 29.5 μmol, 25.9% yield, 99.6% purity) was obtained as a white solid. LC-MS (M+H)⁺ 601.1. ¹H NMR (400 MHz, DMSO-d₆) δ 13.62-12.23 (m, 1H), 8.42-8.40 (m, 1H), 8.24-8.21 (m, 1H), 7.88 (d, J=8.0 Hz, 1H), 7.68 (t, J=8.0 Hz, 1H), 7.47-7.34 (m, 4H), 5.83 (dd, J₁=8.0 Hz, J₂=1.6 Hz, 1H), 4.74-4.63 (m, 1H), 3.83-3.73 (m, 1H), 3.55-3.51 (m, 1H), 3.35 (s, 3H), 2.97-2.84 (m, 2H), 2.76-2.61 (m, 2H), 2.29-2.16 (m, 2H), 2.10-2.05 (m, 1H), 1.88-1.76 (m, 1H), 1.66-1.52 (m, 2H).

### Example 5: Synthesis of Compound 50

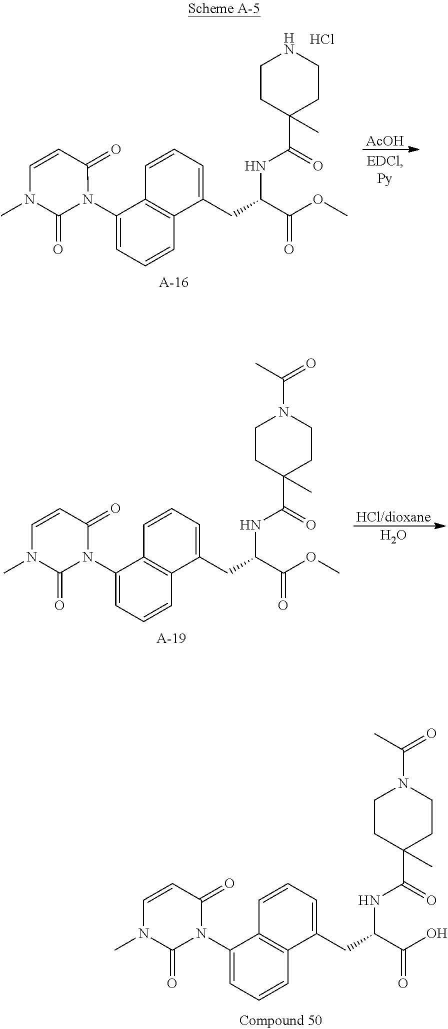

Step 1: To a solution of compound A-16 (100 mg, 160 μmol, 82.5% purity, 1.00 eq, HCl) and acetic acid (11.5 mg, 192 μmol, 10.9 uL, 1.20 eq) in pyridine (2.00 mL) was added EDCI (92.1 mg, 480 μmol, 3.00 eq). The mixture was stirred at 25° C. for 12 hrs. The reaction mixture was concentrated under reduced pressure to give a residue. Compound A-19 (135 mg, crude) was obtained as yellow oil. LC-MS (M+H)⁺ 521.1.

Step 2: To a solution of compound A-19 (100 mg, 192 μmol, 1.00 eq) in H₂O (1.00 mL) was added HCl/dioxane (4.00 M, 2.02 mL, 42.1 eq). The mixture was stirred at 60° C. for 2 hrs. The reaction mixture was concentrated under reduced pressure to give a residue. The residue was purified by Prep-HPLC (column: Phenomenex luna C18 150*25 mm*10 um; mobile phase: [water (HCl)-ACN]; B %: 6%-36%, 10 min). Compound 50 (18.41 mg, 35.7 μmol, 18.6% yield, 98.3% purity) was obtained as a off-white solid ¹H NMR (400 MHz, DMSO-d₆) δ 8.21 (t, J=6.4 Hz, 1H), 7.97 (t, J=8.0 Hz, 1H), 7.88 (d, J=8.0 Hz, 1H), 7.67 (t, J=8.0 Hz, 1H), 7.47-7.36 (m, 4H), 5.83 (dd, J₁=7.6 Hz, J₂=3.2 Hz, 1H), 4.65-4.55 (m, 1H), 3.86-3.68 (m, 2H), 3.48-3.40 (m, 2H), 3.35 (d, J=2.8 Hz, 3H), 3.05-2.97 (m, 1H), 2.82-2.72 (m, 1H), 1.98-1.94 (m, 1H), 1.92 (d, J=4.4 Hz, 3H), 1.86 (d, J=2.4 Hz, 1H), 1.26-1.12 (m, 2H), 0.98 (dd, J₁=11.2 Hz, J₂=2.8 Hz, 3H). LC-MS (M+H)⁺ 507.1.

### Example 6: Synthesis of Compound 60

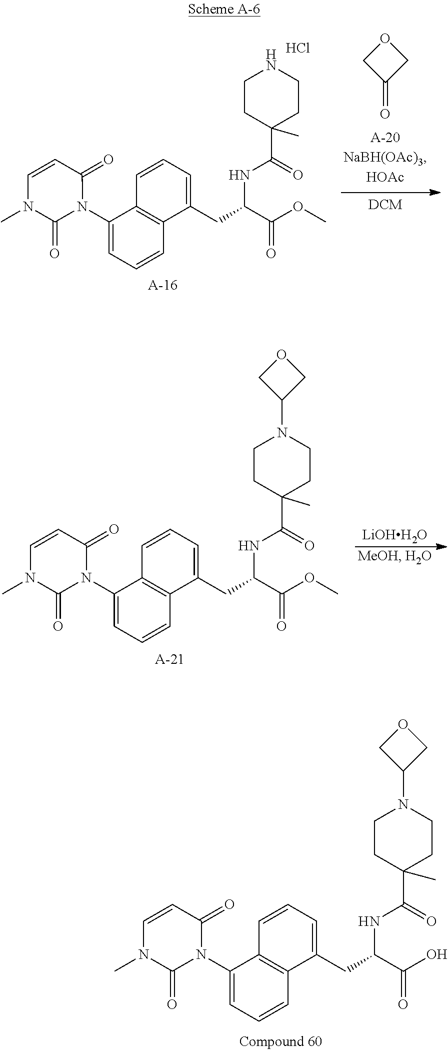

Step 1: To a solution of compound A-16 (60.0 mg, 116 μmol, 1.00 eq, HCl) and compound A-20 (25.2 mg, 349 μmol, 3.00 eq) in DCM (2.00 mL) was added HOAc (69.9 ug, 1.17 μmol, 6.66e-2 μL, 0.0100 eq). The mixture was stirred at 25° C. for 0.5 hr. Then NaBH(OAc)₃ (49.4 mg, 233 μmol, 2.00 eq) was added at 25° C. The mixture was stirred at 40° C. for 2 hrs. The reaction mixture was diluted with sat.aq. NaHCO₃ (30 mL) and extracted with EtOAc (10 mL*3). The combined organic layers were washed with brine (10 mL*2), dried over Na₂SO₄, filtered and concentrated under reduced pressure to give a residue. The residue was purified by Prep-TLC (Dichloromethane:Methanol=10:1). Compound A-21 (40.0 mg, 72.9 μmol, 62.6% yield, 97.5% purity) was obtained as a yellow solid. LC-MS (M+H)⁺ 535.3. ¹H NMR (400 MHz, CDCl₃) δ 8.23 (d, J=8.8 Hz, 1H), 7.66 (t, J=8.0 Hz, 1H), 7.50 (d, J=8.4 Hz, 1H), 7.43-7.38 (m, 2H), 7.34 (d, J=8.0 Hz, 1H), 7.31-7.28 (m, 1H), 6.40 (d, J=7.2 Hz, 1H), 5.93 (dd, J₁=8.0 Hz, J₂=1.2 Hz, 1H), 5.02-4.96 (m, 1H), 4.62-4.53 (m, 4H), 3.66 (s, 3H), 3.63-3.59 (m, 2H), 3.48 (s, 3H), 3.37-3.32 (m, 1H), 2.38-2.30 (m, 2H), 2.00-1.80 (m, 4H), 1.51-1.44 (m, 2H), 1.09 (s, 3H).

Step 2: To a solution of compound A-21 (30.0 mg, 54.7 μmol, 97.5% purity, 1 eq) in MeOH (0.50 mL) was added a solution of LiOH·H₂O (2.30 mg, 54.7 μmol, 1.00 eq) in H₂O (0.5 mL). The mixture was stirred at 25° C. for 2 hrs. The reaction mixture was concentrated under reduced pressure to give a residue. The residue was purified by Prep-HPLC (column: Welch Xtimate C 18 150*25 mm*5 um; mobile phase: [water (NH₃H₂O)-ACN]; B %: 0%-21%, 8 min). Compound 60 (27.18 mg, 52.2 μmol, 95.4% yield, 100% purity) was obtained as a yellow oil. ¹H NMR (400 MHz, DMSO-d₆) δ 8.30 (d, J=8.8 Hz, 1H), 7.88 (d, J=8.0 Hz, 1H), 7.64-7.60 (m, 1H), 7.55 (d, J=7.2 Hz, 1H), 7.41-7.32 (m, 4H), 5.84 (dd, J₁=7.2 Hz, J₂=3.2 Hz, 1H), 4.52-4.41 (m, 3H), 4.33-4.30 (m, 2H), 3.69-3.63 (m, 1H), 3.40 (dd, J₁=8.8 Hz, J₂=6.0 Hz, 1H), 3.36 (d, J=4.4 Hz, 3H), 3.20-3.13 (m, 1H), 2.22-2.18 (m, 2H), 1.92-1.75 (m, 3H), 1.68-1.61 (m, 1H), 1.29-1.23 (m, 2H), 0.96 (s, 3H). LC-MS (M+H)⁺ 521.3.

### Example 7: Synthesis of Compound 55

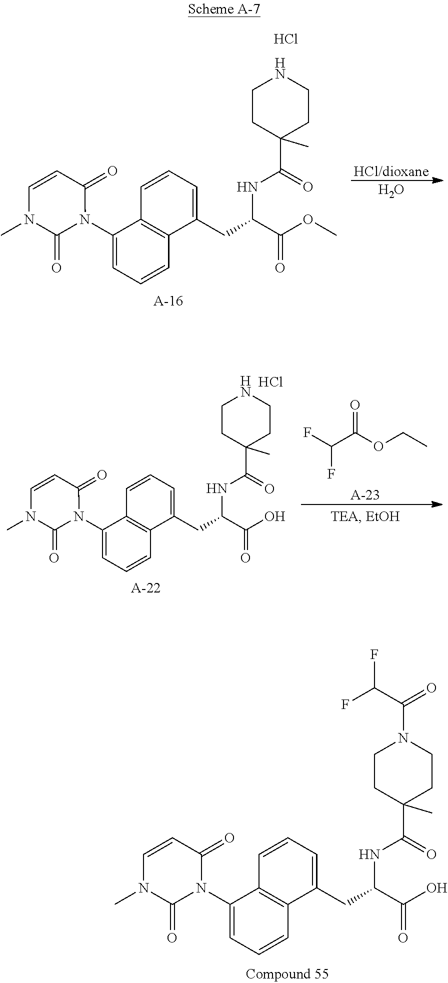

Step 1: To a solution of compound A-16 (100 mg, 194 μmol, 1.00 eq, HCl) in H₂O (2.00 mL) was added HCl/dioxane (4 M, 2.00 mL, 41.2 eq). The mixture was stirred at 60° C. for 2 hrs. The reaction mixture was concentrated under reduced pressure to give a residue. Compound A-22 (120 mg, crude, HCl) was obtained as a yellow oil. LC-MS (M+H)⁺ 465.1.

Step 2: To a solution of compound A-22 (120 mg, 239 μmol, 1.00 eq, HCl) and compound A-23 (594 mg, 4.79 mmol, 20.0 eq) in EtOH (2.00 mL) was added TEA (121 mg, 1.20 mmol, 166 μL, 5.00 eq). The mixture was stirred at 85° C. for 24 hrs. The mixture was taken up into a microwave tube. The sealed tube was heated at 100° C. for 2 hrs under microwave. The reaction mixture was filtered and concentrated under reduced pressure to give a residue. The residue was purified by prep-HPLC (column: Phenomenex luna C18 150*25 mm*10 um; mobile phase: [water (HCl)-ACN]; B %: 12%-42%, 10 min). Compound 55 (33.79 mg, 61.6 μmol, 25.7% yield, 98.9% purity) was obtained as a yellow gum. ¹H NMR (400 MHz, DMSO-d₆) δ 12.81 (s, 1H), 8.25-8.20 (m, 1H), 8.04 (dd, J₁=8.0 Hz, J₂=4.8 Hz, 1H), 7.88 (d, J=8.0 Hz, 1H), 7.67 (t, J=8.0 Hz, 1H), 7.45-7.37 (m, 4H), 7.23-6.50 (m, 1H), 5.83 (dd, J₁=8.0 Hz, J₂=2.4 Hz, 1H), 4.67-4.56 (m, 1H), 3.89-3.69 (m, 2H), 3.57-3.40 (m, 2H), 3.35-3.34 (m, 3H), 3.10-2.63 (m, 2H), 2.07-1.92 (m, 2H), 1.30-1.16 (m, 2H), 1.01-0.99 (m, 3H). LC-MS (M+H)⁻ 543.3.

### Example 8: Synthesis of Compound 159

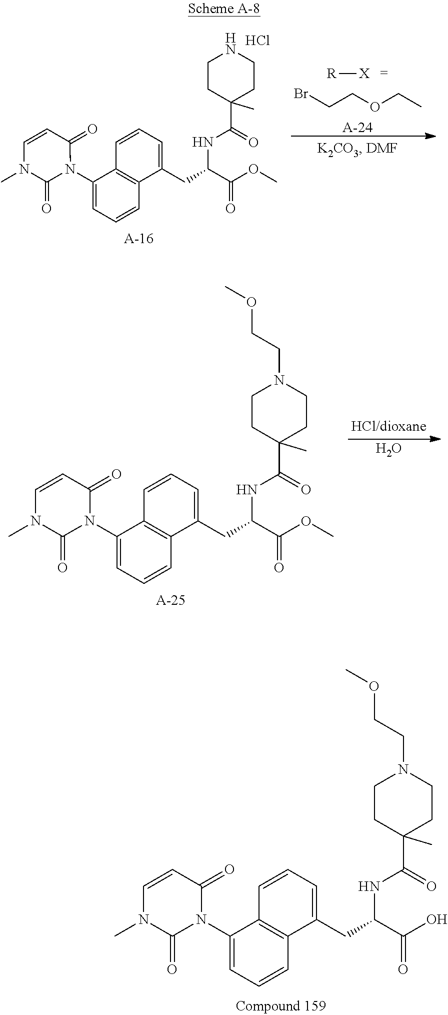

Step 1: To a solution of compound A-16 (100 mg, 160 μmol, 82.5% purity, 1.00 eq, HCl) and compound A-24 (26.7 mg, 192 μmol, 18.1 uL, 1.20 eq) in DMF (2.00 mL) was added K₂CO₃ (44.3 mg, 320 μmol, 2.00 eq). The mixture was stirred at 60° C. for 4 hrs. The reaction mixture was concentrated under reduced pressure to give a residue. The residue was diluted with H₂O (20 mL) and extracted with Ethyl acetate (20 mL*3). The combined organic layers were washed with brine (20 mL*2), dried over Na₂SO₄, filtered and concentrated under reduced pressure to give a residue. Compound A-25 (90.0 mg, crude) was obtained as yellow oil. LC-MS (M+H)⁺ 537.3.

Step 2: To a solution of compound A-25 (80.0 mg, 149 μmol, 1.00 eq) in H₂O (2.00 mL) was added HCl/dioxane (4.00 M, 2.00 mL, 53.7 eq) at 0° C. The mixture was stirred at 60° C. for 2 hrs. The reaction mixture was filtered and concentrated under reduced pressure to give a residue. Then the residue was diluted with H₂O (10 mL), and added aq. NH₃·H₂O to adjust pH=7 at 0° C., and concentrated under reduced pressure to give a residue. The residue was purified by Prep-HPLC (column: Waters xbridge 150*25 mm 10 um; mobile phase: [water (NH₄HCO₃)-ACN]; B %: 0%-20%, 11 min). Compound 159 (51.26 mg, 94.8 μmol, 63.6% yield, 96.7% purity) was obtained as off-white solid. LC-MS (M+H)⁺ 523.2. ¹H NMR (400 MHz, DMSO-d₆) δ 8.25 (d, J=4.4 Hz, 1H), 7.88 (d, J=8.0 Hz, 1H), 7.73 (d, J=8.0 Hz, 1H), 7.65 (t, J=7.6 Hz, 1H), 7.44-7.35 (m, 4H), 5.83 (dd, J₁=8.0 Hz, J₂=3.2 Hz, 1H), 4.56-4.51 (m, 1H), 3.71-3.67 (m, 1H), 3.45-3.40 (m, 2H), 3.38-3.35 (m, 4H), 3.19 (s, 3H), 2.67-2.64 (m, 2H), 2.40 (t, J=5.6 Hz, 2H), 2.19-2.14 (m, 1H), 1.98-1.89 (m, 3H), 1.33-1.28 (m, 2H), 0.95 (s, 3H).

### Example 9: Synthesis of Compound 157

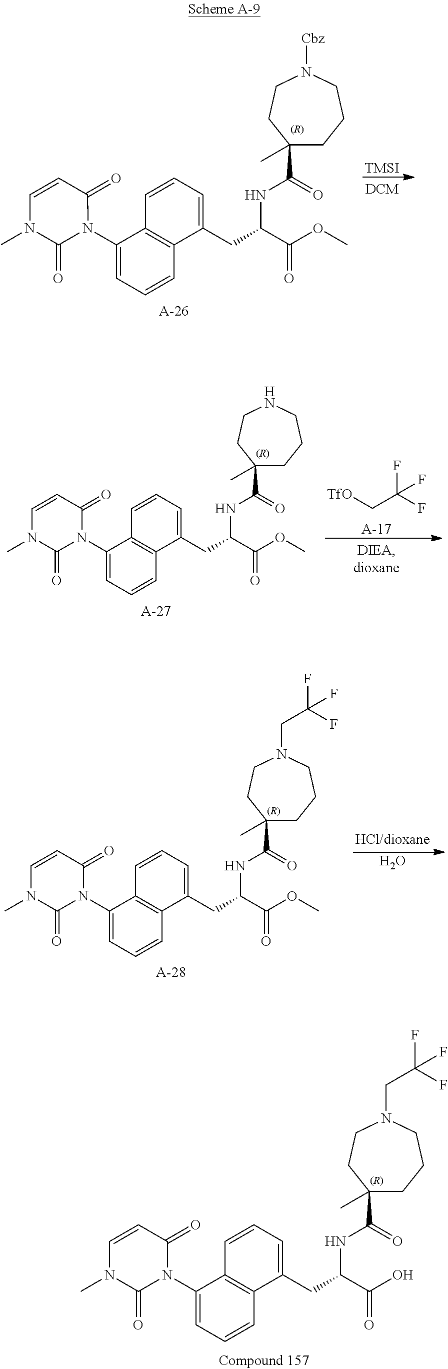

Step 1: To a solution of compound A-26 (70.0 mg, 111 μmol, 99.4% purity, 1.00 eq) in DCM (3.00 mL) was added drop-wise TMSI (44.4 mg, 222 μmol, 30.2 uL, 2.00 eq), then the mixture was stirred at 25° C. for 0.5 hr. The reaction mixture was concentrated under reduced pressure to remove DCM. The residue was diluted with HCl (1.00 M, 5 mL) and extracted with ethyl acetate (5 mL*3). The aqueous phase was adjusted pH to 9 with sat.aq. Na₂CO₃ and extracted with ethyl acetate (5 mL*3). The second organic layers were dried over Na₂SO₄, filtered and concentrated under reduced pressure to give a residue. Compound A-27 (30.0 mg, 56.0 μmol, 50.4% yield, 91.9% purity) was obtained as white solid. LC-MS (M+H)⁺: 493.3.

Step 2: To a solution of compound A-27 (30.0 mg, 56.0 μmol, 91.9% purity, 1.00 eq) in dioxane (2.00 mL) was added DIEA (14.5 mg, 112 μmol, 19.5 uL, 2.00 eq) and compound A-17 (13.0 mg, 56.0 μmol, 1.00 eq) at 25° C., then the mixture was stirred at 90° C. for 12 hrs. The residue was diluted with H₂O (20 mL) and extracted with ethyl acetate (20 mL*3). The combined organic layers were washed with brine (20 mL*3), dried over Na₂SO₄, filtered and concentrated under reduced pressure to give a residue. Compound A-28 (30 mg, 52.21 μmol, 93.28% yield) was obtained as yellow solid. LC-MS (M+H)⁺: 575.4.

Step 3: To a solution of compound A-28 (25.0 mg, 43.5 μmol, 1.00 eq) in H₂O (0.500 mL) was added HCl/dioxane (4.00 M, 5.00 mL, 460 eq), then the mixture was stirred at 60° C. for 1 hr. The mixture was concentrated under reduced pressure to give a residue. The residue was purified by prep-HPLC (column: Welch Xtimate C18 150*25 mm*5 um; mobile phase: [water (NH₃H₂O)-ACN]; B %: 5%-35%, 8 min). Compound 157 (11.78 mg, 21.0 μmol, 48.2% yield, 99.7% purity) was obtained as white solid. ¹H NMR (400 MHz, DMSO-d₆) δ 8.34 (br d, J=8.4 Hz, 1H), 7.87 (d, J=8.0 Hz, 1H), 7.57 (t, J=7.6 Hz, 1H), 7.39-7.23 (m, 5H), 5.83 (dd, J₁=7.6 Hz, J₂=0.8 Hz, 1H), 4.29-4.19 (m, 1H), 3.61-3.54 (m, 1H), 3.35 (br s, 4H), 3.18-3.12 (m, 2H), 2.75-2.69 (m, 1H), 2.65-2.61 (m, 2H), 1.98-1.88 (m, 2H), 1.58-1.30 (m, 5H), 0.96 (s, 3H). LC-MS (M+H)⁺: 561.3.

The following compounds, set forth in Table 5, were also prepared using the synthetic procedures of Schemes A-1 to A-9, or analogous procedures thereto:

TABLE 5

| Compound | |
|---|---|
| No. | Characterization Data |
| 1 | 479.9 [M + 1] |
| 6 | 602.2 [M + 1] |
| 7 | 531.1 [M + 1] |
| 8 | 627.1 [M + 1] |
| 9 | 635.2 [M + 1] |
| 10 | 465.9 [M + 1] |
| 11 | 527.4 [M + 1] |
| 12 | 627.1 [M + 1] |
| 13 | 518.4 [M + 1] |
| 14 | 512.1 [M + 1] |
| 15 | 600.2 [M − 1] (negative ion mode) |
| 16 | 602.2 [M + 1] |
| 17 | 477.2 [M + 1] |
| 18 | 547.3 [M + 1] |
| 19 | 658.1 [M + 1] |
| 20 | 714.4 [M + 1] |
| 21 | 646.4 [M + 1] |
| 22 | 727.6 [M + 1] |
| 26 | 548.0 [M + 1] |
| 27 | 533.1 [M + 1] |
| 28 | 510.2 [M + 1] |
| 29 | 548.1 [M + 1] |
| 30 | 536.2 [M − 1] (negative ion mode) |
| 31 | 546.1 [M + 1] |
| 32 | 503.1 [M − 1] (negative ion mode) |
| 34 | 577.3 [M − 1] (negative ion mode) |
| 40 | 545.2 [M + 1] |
| 41 | 647.0 [M + 1] |
| 42 | 728.1 [M + 1] |
| 43 | 571.1 [M + 1] |
| 48 | 698.1 [M + 1] |
| 50 | 507.1 [M + 1] |
| 51 | 476.1 [M + 1] |
| 52 | 490.2 [M + 1] |
| 53 | 502.1 [M + 1] |
| 54 | 561.1 [M + 1] |
| 55 | 543.3 [M + 1] |
| 56 | 529.2 [M + 1] |
| 57 | 507.2 [M + 1] |
| 58 | 529.2 [M + 1] |
| 59 | 521.1 [M + 1] |
| 60 | 521.3 [M + 1] |
| 61 | 601.1 [M + 1] |
| 63 | 500.2 [M + 1] |
| 65 | 538.1 [M + 1] |
| 66 | 514.2 [M + 1] |
| 69 | 430.1 [M + 1] |
| 70 | 515.0 [M + 1] |
| 71 | 533.2 [M + 1] |
| 72 | 623.2 [M + 1] |
| 73 | 588.1 [M + 1] |
| 74 | 568.0 [M + 1] |
| 75 | 584.1 [M + 1] |
| 77 | 593.9 [M + 1] |
| 79 | 538.1 [M + 1] |
| 81 | 623.2 [M + 1] |
| 83 | 563.1 [M + 1] |
| 84 | 549.2 [M + 1] |
| 94 | 659.1 [M + 1] |
| 95 | 658.9 [M + 1] |
| 108 | 632.9 [M + 1] |
| 109 | 577.3 [M + 1] |
| 110 | 497.1 [M + 1] |
| 111 | 573.2 [M + 1] |
| 112 | 605.3 [M + 1] |
| 114 | 576.9 [M + 1] |
| 120 | 577.2 [M + 1] |
| 122 | 544.1 [M + 1] |
| 123 | 546.2 [M + 1] |
| 124 | 584.4 [M + 1] |
| 126 | 600.1 [M − 1] (negative ion mode) |
| 128 | 724.7 [M + 1] |
| 130 | 659.3 [M + 1] |
| 131 | 505.3 [M + 1] |
| 132 | 601.3 [M + 1] |
| 133 | 531.3 [M + 1] |
| 134 | 599.1 [M + 1] |
| 135 | 559.3 [M + 1] |
| 138 | 510.3 [M + 1] |
| 139 | 508.0 [M + 1] |
| 144 | 602.3 [M + 1] |
| 145 | 494.0 [M + 1] |
| 147 | 712.2 [M + 1] |
| 152 | 561.1 [M + 1] |
| 153 | 575.2 [M + 1] |
| 154 | 559.2 [M + 1] |
| 155 | 559.2 [M + 1] |
| 156 | 589.4 [M + 1] |
| 157 | 561.3 [M + 1] |
| 158 | 561.3 [M + 1] |
| 159 | 523.2 [M + 1] |
| 160 | 493.2 [M + 1] |
| 161 | 464.2 [M + 1] |
| 162 | 492.2 [M + 1] |
| 163 | 558.2 [M + 1] |
| 164 | 551.1 [M + 1] |
| 165 | 544.2 [M + 1] |
| 166 | 559.1 [M + 1] |
| 167 | 561.1 [M + 1] |
| 168 | 575.2 [M + 1] |
| 170 | 633.2 [M + 1] |
| 173 | 600.2 [M + 1] |
| 174 | 594.2 [M + 1] |
| 176 | 562.2 [M + 1] |
| 180 | 574.2 [M + 1] |
| 181 | 588.0 [M + 1] |

### Example 10: Synthesis of Compound 142

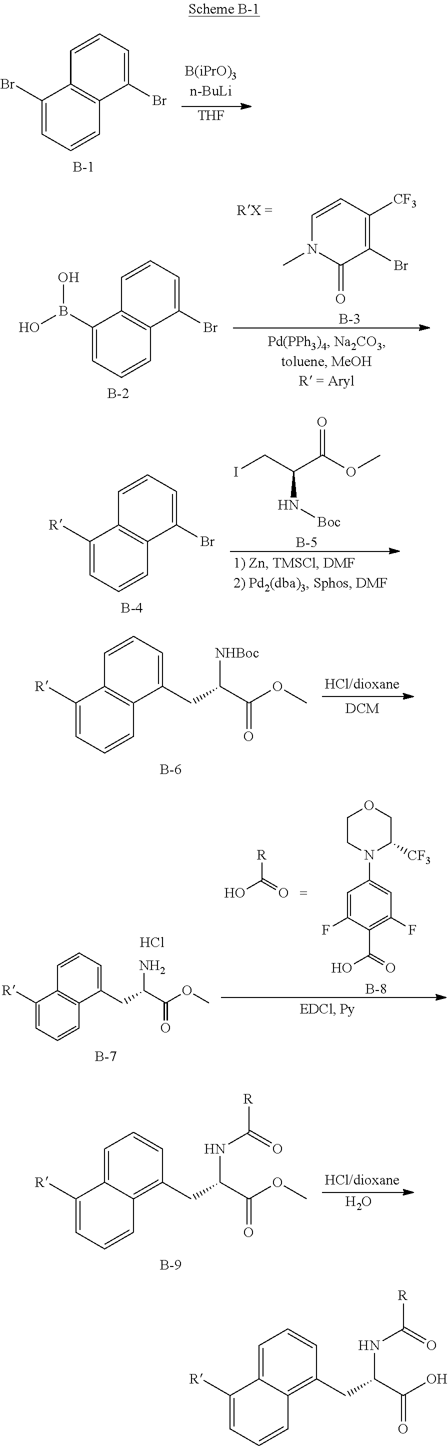

Step 1: A solution of compound B-1 (5.00 g, 17.5 mmol, 1.00 eq) in THF (200 mL) was cooled to −78° C., n-BuLi (2.50 M, 7.69 mL, 1.10 eq) was added to the mixture, after stirred at −78° C. for 2 hrs, triisopropyl borate (4.93 g, 26.2 mmol, 6.03 mL, 1.50 eq) was added to the mixture. The resulting mixture was stirred for 18 hrs at 25° C. The mixture was quenched by 1.00 M HCl (100 mL) at 0° C., stirred for 0.5 hr. The mixture was extracted with MTBE (100 mL*2). The organic layers were dried over Na₂SO₄, concentrated under vacuum to give a residue. The residue was triturated with hexane:toluene (1:1, 150 mL) for 1 hr, filtered to the filter cake. Compound B-2 (3.00 g, 11.9 mmol, 68.4% yield) was obtained as a white solid. ¹H NMR (400 MHz, DMSO-d₆). δ 8.55-8.32 (m, 3H), 8.20-8.14 (m, 1H), 7.88-7.77 (m, 2H), 7.70-7.62 (m, 1H), 7.43 (m, 1H).

Step 2: Compound B-3 (200 mg, 781 μmol, 1.00 eq), compound B-2 (216 mg, 859 μmol, 1.10 eq) and Na₂CO₃ (166 mg, 1.56 mmol, 2.00 eq), Pd(PPh₃)⁺ (90.3 mg, 78.1 μmol, 0.10 eq) were added into a microwave tube with toluene (2.00 mL) and MeOH (2.00 mL). The sealed tube was heated at 100° C. for 60 min under microwave. The mixture was diluted with H₂O (20 mL) and extracted with DCM (20 mL*2). The combined organic layers were dried over Na₂SO₄, filtered and concentrated under reduced pressure to give a residue. The residue was purified by column chromatography (SiO₂, Petroleum ether:Ethyl acetate=100:1 to 2:1) (Petroleum ether:Ethyl acetate=2:1, R_{f}(P1)=0.40). The residue was purified by Prep-HPLC (neutral condition; column: YMC Triart C18 250*50 mm*7 um; mobile phase: [water (NH₄HCO₃)-ACN]; B %: 50%-80%, 10 min) again. Compound B-4 (247 mg, 646 μmol, 82.7% yield) was obtained as a white solid. LC-MS (M+H)⁻ 383.8. ¹H NMR (400 MHz, CDCl₃) δ 8.33 (d, J=8.4 Hz, 1H), 7.77 (d, J=7.6 Hz, 1H), 7.65-7.60 (m, 1H), 7.54 (d, J=7.2 Hz, 1H), 7.43-7.34 (m, 2H), 7.26-7.22 (m, 1H), 6.53 (d, J=7.2 Hz, 1H), 3.63 (s, 3H).

Step 3: To Zn (1.51 g, 23.1 mmol, 1.90 eq) was added TMSCl (396 mg, 3.65 mmol, 463 μL, 0.30 eq) in DMF (20 mL) and stirred at 20-25° C. for 30 min. And the supernatant liquor was removed by syringe. Then a mixture of compound B-5 (4.00 g, 12.2 mmol, 1.00 eq) in DMF (20 mL) was added to the precipitate. And the resulting mixture was stirred at 20° C. for 2 hrs. Compound [(2R)-2-(tert-butoxycarbonylamino)-3-methoxy-3-oxo-propyl]-iodo-zinc (4.79 g, 12.1 mmol, 100% yield) was obtained as a colorless liquid, which was used to next step directly. To a solution of compound B-4 (247 mg, 646 μmol, 1.00 eq) in DMF (2.00 mL) was added compound [(2R)-2-(tert-butoxycarbonylamino)-3-methoxy-3-oxo-propyl]-iodo-zinc (765 mg, 1.94 mmol, 3.00 eq), Pd₂(dba)₃ (59.2 mg, 64.6 μmol, 0.10 eq) and SPhos (53.1 mg, 129 μmol, 0.20 eq) under N₂ atmosphere. The mixture was stirred at 75-80° C. for 12 hrs. The mixture was diluted with H₂O 20 mL and extracted with ethyl acetate (20 mL*2), the combined organic layers were dried over Na₂SO₄, filtered and concentrated under reduced pressure to give a residue. The residue was purified by column chromatography (SiO₂, Petroleum ether:Ethyl acetate=100:1 to 1:1) (Petroleum ether:Ethyl acetate=1:1, R_{f}(P1)=0.40). Compound B-6 (250 mg, 496 μmol, 76.7% yield) was obtained as a yellow solid. LC-MS: (M-55)⁺ 449.2.

Step 4: To a solution of compound B-6 (240 mg, 478 μmol, 1.00 eq) in DCM (3.00 mL) was added HCl/dioxane (4.00 M, 6.00 mL, 50.4 eq) at 0° C. The mixture was stirred at 20-25° C. for 1 hr. The reaction mixture was concentrated under reduced pressure to give a residue. The crude product was used into the next step without further purification. Compound B-7 (209 mg, 474 μmol, 99.7% yield, HCl) was obtained as a yellow gum. LC-MS: (M+H)⁺ 405.1.

Step 5: To a solution of compound B-7 (100 mg, 227 μmol, 1.00 eq, HCl) in pyridine (4.00 mL) was added compound B-8 (84.7 mg, 272 μmol, 1.20 eq) and EDCI (86.9 mg, 454 μmol, 2.00 eq), the reaction mixture was stirred at 20-25° C. for 2 hrs. The reaction mixture was concentrated under reduced pressure to remove solvent to give a residue. The residue was purified by Prep-TLC (SiO₂, Dichloromethane:Methanol=10:1) (Dichloromethane:Methanol=10:1, R_{f}(P1)=0.50). Compound B-9 (139 mg, 199 μmol, 87.8% yield) was obtained as a white solid. LC-MS (M+H)+698.4.

Step 6: To a solution of compound B-9 (139 mg, 199 μmol, 1.00 eq) in dioxane (1.50 mL) was added HCl (6.00 M, 5.00 mL, 150 eq) and stirred at 60-65° C. for 6 hrs. The residue was purified by Prep-HPLC (FA condition; Phenomenex luna C18 150*25 mm*10 um; mobile phase: [water (FA)-ACN]; B %: 36%-66%, 10.5 min). Compound 142 (90.77 mg, 133 μmol, 66.6% yield, 100% purity) was obtained as a white solid. LC-MS (M+H)⁺ 684.3. ¹H NMR (400 MHz, DMSO-d₆+D₂O). δ 8.15 (brd, J=8.8 Hz, 1H), 7.98 (d, J=7.2 Hz, 1H), 7.64-7.56 (m, 1H), 7.42-7.37 (m, 1H), 7.35-7.19 (m, 3H), 6.74-6.66 (m, 2H), 6.65-6.62 (m, 1H), 4.81-4.62 (m, 2H), 4.15-4.11 (m, 1H), 3.74-3.62 (m, 3H), 3.56-3.45 (m, 4H), 3.41-3.28 (m, 2H), 3.27-3.15 (m, 1H).

The following compounds, set forth in Table 6, were also prepared using the synthetic procedures of Scheme B-1, or analogous procedures thereto:

TABLE 6

| Compound | |
|---|---|
| No. | Characterization Data |
| 5 | 515.2 [M + 1] |
| 23 | 637.2 [M + 1] |
| 37 | 553.3 [M + 1] |
| 38 | 583.3 [M + 1] |
| 39 | 633.1 [M + 1] |
| 46 | 714.2 [M + 1] |
| 67 | 684.1 [M + 1] |
| 113 | 698.2 [M + 1] |
| 115 | 531.3 [M + 1] |
| 129 | 718.2 [M + 1] |
| 140 | 633.1 [M + 1] |
| 143 | 765.5 [M + 1] |
| 146 | 714.2 [M + 1] |
| 196 | 531.1 [M + 1] |

### Example 11: Synthesis of Compound 182

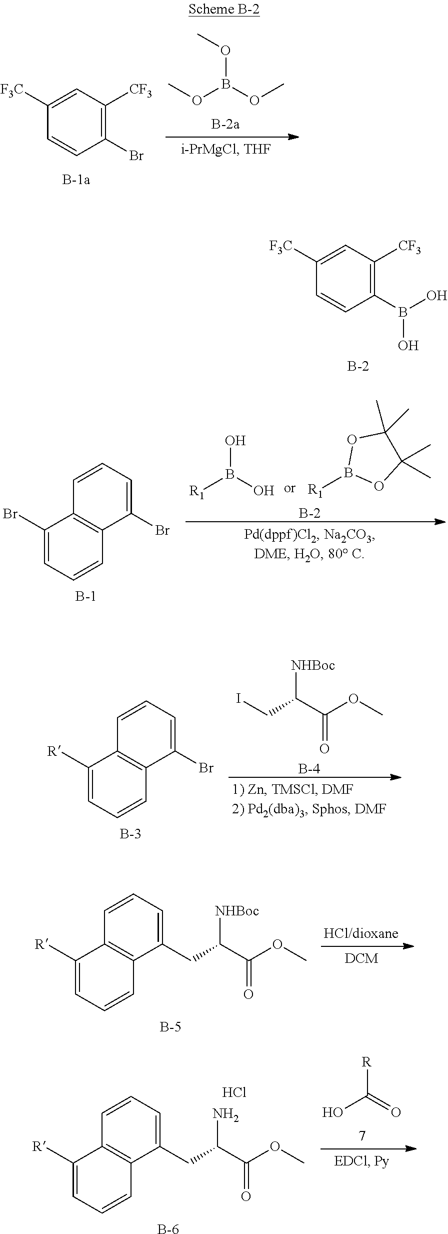

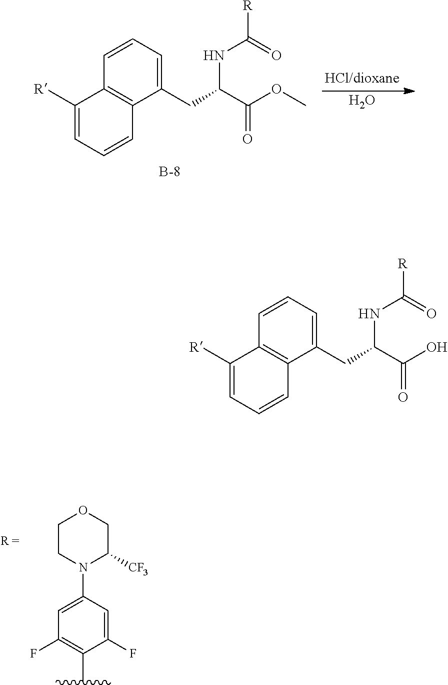

Intermediate B-2: To a solution of compound B-1a (8.00 g 27.3 mmol, 4.60 mL, 1.00 eq) in THF (80 mL) was added i-PrMgCl (2.00 M, 16.4 mL, 1.20 eq) at −10° C. The mixture was stirred at −10° C. for 2 hrs. The resulting mixture was added to a cold solution of compound B-2a (7.09 g 68.3 mmol, 7.71 mL, 2.50 eq) in THF (20 mL) at −10° C. Then the mixture was stirred at −10° C. for another 12 hrs. The reaction was quenched by 1M HCl (50 mL) at 0° C., stirred for 0.5 hr. The mixture was extracted with DCM (50 mL*2). The organic layers were dried over Na 2SO₄, concentrated under vacuum to give a residue. The residue was triturated with hexane (50 mL) and filtered. Compound B-2 (6.50 g, 25.2 mmol, 92.3% yield) was obtained as white solid.

Step 1: A mixture of compound B-2 (1.66 g, 5.82 mmol, 1.50 eq), compound B-1 (1.66 g, 5.82 mmol, 1.50 eq), Pd(PPh₃)⁺ (896 mg, 775 μmol, 0.200 eq) and K₂CO₃ (1.07 g, 7.75 mmol, 2.00 eq) in dioxane (20 mL) and H₂O (5 mL) was degassed and purged with N₂ for three times. Then the mixture was stirred at 90° C. for 12 hrs under N₂ atmosphere. The reaction mixture was concentrated under reduced pressure to remove dioxane. The residue was diluted with H₂O (30 mL) and extracted with ethyl acetate (30 mL*3). The combined organic layers were washed with brine (30 mL*3), dried over Na₂SO₄, filtered and concentrated under reduced pressure to give a residue. The crude product was triturated with Petroleum ether/Ethyl acetate=3/1 at 20° C. for 60 min, the filter was concentrated under reduced pressure to give desired compound (1.00 g). The cake was concentrated under reduced pressure to give a crude residue (1.20 g). The crude residue was purified by reverse-phase HPLC (FA condition) and give desired compound (0.500 g). Compound B-3 (1.50 g, 3.58 mmol, 92.3% yield) was obtained as white solid. LC-MS: (2M+H)⁻ 837.0. ¹H NMR (400 MHz, DMSO-d₆) δ 8.29 (d, J=8.4 Hz, 1H), 8.25 (s, 1H), 7.94 (d, J=7.2 Hz, 1H), 7.82-7.74 (m, 1H), 7.72 (d, J=8.0 Hz, 1H), 7.61 (t, J=8.0 Hz, 1H), 7.55 (d, J=7.2 Hz, 1H), 7.38 (t, J=8.0 Hz, 1H), 7.25 (d, J=8.4 Hz, 1H).

Step 2: Zn (5.96 g, 91.2 mmol, 3.00 eq) was added to a three necked flask and heated at 110° C. under vacuum for 10 mins, then cooled to 25° C. To a flask was added a solution of TMSCl (992 mg, 9.13 mmol, 1.16 mL, 0.300 eq) in DMF (20 mL) and the inner temperature was gradually rose to 30° C., the mixture was stirred at 25-30° C. for 20 mins. And the supernatant liquor was removed by syringe, then a mixture of compound B-4 (10.0 g, 30.4 mmol, 1.00 eq) in DMF (30 mL) was added to the precipitate, and the inner temperature was rapidly rose from 25° C. to 45° C., and the resulting mixture was stirred at 25-45° C. for 1 hr. Compound [(2R)-2-(tert-butoxycarbonylamino)-3-methoxy-3-oxo-propyl]-iodo-zinc (11.9 g, 30.4 mmol, 100% yield) was obtained as a colorless liquid, which was directly used in next step. To a solution of compound B-3 (1.50 g, 3.58 mmol, 1.00 eq), SPhos (294 mg, 716 μmol, 0.200 eq) and Pd₂(dba)₃ (328 mg, 358 μmol, 0.100 eq) in DMF (20 mL) was added [(2R)-2-(tert-butoxycarbonylamino)-3-methoxy-3-oxo-propyl]-iodo-zinc (4.24 g 10.7 mmol, 3.00 eq), then the mixture was stirred at 80° C. for 12 hrs under N₂ atmosphere. The residue was diluted with H₂O (50 mL) and extracted with ethyl acetate (30 mL*2). The combined organic layers were washed with brine (30 mL*2), dried over Na₂SO₄, filtered and concentrated under reduced pressure to give a residue. The residue was purified by column chromatography (SiO₂, Petroleum ether/Ethyl acetate=100/1 to 1/1). TLC (Petroleum ether/Ethyl acetate=10/1). Compound B-5 (1.70 g, 2.92 mmol, 81.5% yield, 92.9% purity) was obtained as yellow oil. LC-MS (M-99)⁺ 442.1.

Step 3: To a solution of compound B-5 (1.70 g, 2.92 mmol, 92.9% purity, 1.00 eq) in DCM (10 mL) was added HCl/dioxane (4.00 M, 5.00 mL, 6.86 eq), then the mixture was stirred at 15° C. for 1 hr. The mixture was concentrated under reduced pressure to give a residue. Compound B-6 (1.30 g, 2.72 mmol, 93.3% yield, HCl) was obtained as yellow solid. LC-MS: (M+H)⁺ 442.0.

Step 4: To a solution of compound B-6 (70.0 mg, 147 μmol, 1.00 eq, HCl) and compound B-7 (54.7 mg, 176 μmol, 1.20 eq) in pyridine (5 mL) was added EDCI (56.2 mg, 293 μmol, 2.00 eq), then the mixture was stirred at 15° C. for 12 hrs. The residue was diluted with sat.aq. NaHCO₃ (30 mL) and extracted with ethyl acetate (15 mL*3). The combined organic layers were washed with brine (15 mL*3), dried over Na₂SO₄, filtered and concentrated under reduced pressure to give a residue. The residue was purified by prep-TLC (SiO₂, DCM/MeOH=10/1). Compound B-8 (100 mg, 126 μmol, 85.8% yield, 92.4% purity) was obtained as white solid. LC-MS: (M+H)⁻ 735.1.

Step 5: To a solution of compound B-8 (100 mg, 126 μmol, 92.4% purity, 1.00 eq) in H₂O (2.00 mL) was added HCl/dioxane (4.00 M, 6.00 mL, 191 eq), then the mixture was stirred at 60° C. for 12 hrs. The mixture was concentrated under reduced pressure to give a residue. The residue was purified by prep-HPLC (column: Phenomenex luna C18 150*25 mm*10 um; mobile phase: [water (FA)-ACN]; B %: 60%-90%, 10 min). Compound 182 (59.88 mg, 82.4 μmol, 65.5% yield, 99.2% purity) was obtained as white solid. LC-MS (M+H)⁻ 721.1. ¹H NMR (400 MHz, DMSO-d₆) δ 12.9 (brs, 1H), 8.98-8.85 (m, 1H), 8.29 (br d, J=8.4 Hz, 1H), 8.24 (brs, 1H), 8.17 (d, J=8.0 Hz, 1H), 7.76 (d, J=8.0 Hz, 1H), 7.72-7.63 (m, 1H), 7.53-7.42 (m, 2H), 7.35 (t, J=8.4 Hz, 1H), 7.10 (d, J=8.4 Hz, 1H), 6.76 (brd, J=11.6 Hz, 2H), 4.97-4.85 (m, 1H), 4.76-4.63 (m, 1H), 4.16 (d, J=12.8 Hz, 1H), 3.95 (dd, J₁=11.6 Hz, J₂=3.6 Hz, 1H), 3.85-3.65 (m, 2H), 3.60-3.51 (m, 1H), 3.48-3.35 (m, 2H), 3.29-3.20 (m, 1H).

The following compounds, set forth in Table 7, were also prepared using the synthetic procedures of Scheme B-2, or analogous procedures thereto:

TABLE 7

| Compound | |
|---|---|
| No. | Characterization Data |
| 47 | 636.1 [M + 1] |
| 104 | 731.3 [M + 1] |
| 148 | 701.2 [M + 1] |
| 149 | 717.1 [M + 1] |
| 150 | 645.1 [M + 1] |
| 151 | 727.2 [M + 1] |
| 171 | 703.2 [M + 1] |
| 172 | 550.1 [M + 1] |
| 197 | 568.2 [M + 1] |

### Example 12: Synthesis of Compound 210

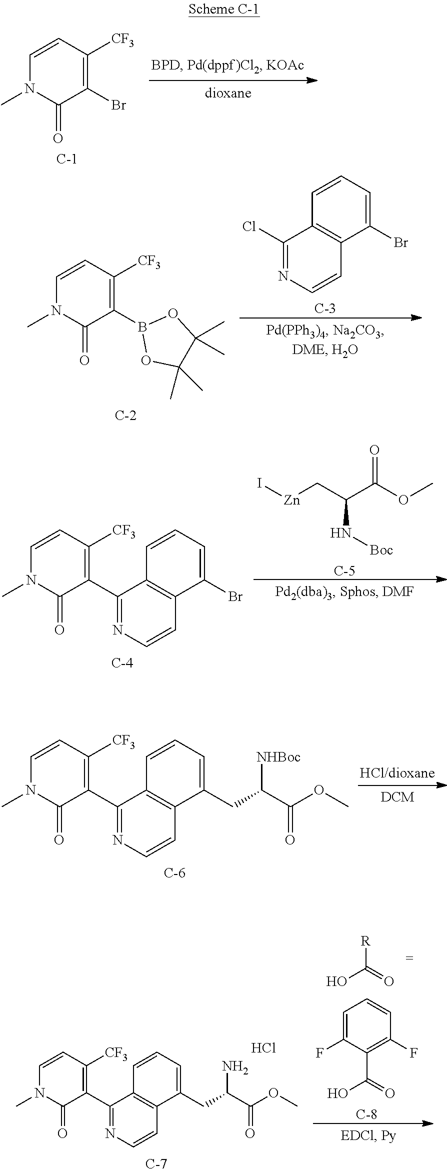

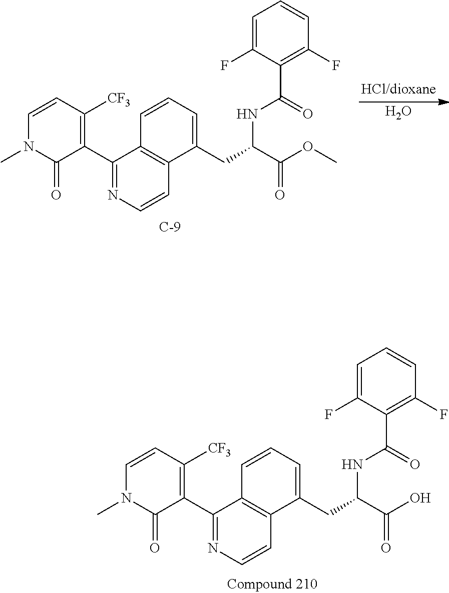

Step 1: A mixture of compound C-1 (3.30 g, 12.9 mmol, 1.00 eq), BPD (13.1 g, 51.6 mmol, 4.00 eq), Pd(dppf)Cl₂ (1.89 g, 2.58 mmol, 0.200 eq) and KOAc (6.32 g, 64.5 mmol, 5.00 eq) in dioxane (50 mL) was degassed and purged with N₂ for 3 times, and then the mixture was stirred at 90° C. for 5 hrs under N₂ atmosphere. The mixture was filtered to get the filtrate. The filtrate was concentrated under vacuum to give a residue. The residue was purified by column chromatography (SiO₂, petroleum ether:ethyl acetate=10:1 to 2:1), TLC (petroleum ether:ethyl acetate=1:1, KMnO₄, R_{f}=0.22). Compound C-2 (2.10 g, 4.57 mmol, 35.5% yield, 66.0% purity) was obtained as yellow oil. LC-MS (M+H)⁺ 304.0. ¹H NMR (400 MHz, CDCl₃) δ 7.39 (d, J=7.2 Hz, 1H), 6.28 (d, J=7.2 Hz, 1H), 3.53 (s, 3H), 1.39 (s, 12H).

Step 2: A mixture of compound C-2 (1.50 g, 3.27 mmol, 66.0% purity, 1.00 eq), compound 3 (800 mg, 3.30 mmol, 1.01 eq), Pd(PPh₃)⁺ (377 mg, 327 μmol, 0.100 eq), Na₂CO₃ (1.04 g, 9.80 mmol, 4.90 mL, 3.00 eq) in DME (10 mL) and H₂O (5 mL) was degassed and purged with N₂ for 3 times, and then the mixture was stirred at 85° C. for 2 hrs under N₂ atmosphere. The mixture was diluted with water (30 mL), extracted with EtOAc (15 mL*3). The organic layers were concentrated under vacuum to give a residue. The residue was purified by column chromatography (SiO₂, petroleum ether:ethyl acetate=10:1 to 0:1). TLC (ethyl acetate, R_{f}=0.27). Compound C-4 (190 mg, 496 μmol, 15.2% yield) was obtained as off-white solid. LC-MS (M+H)⁻ 384.9. ¹H NMR (400 MHz, CDCl₃) δ 8.72 (d, J=6.0 Hz, 1H), 8.09 (d, J=6.0 Hz, 1H), 7.97 (d, J=7.6 Hz, 1H), 7.65-7.59 (m, 2H), 7.39 (t, J=8.0 Hz, 1H), 6.55 (d, J=7.2 Hz, 1H), 3.64 (s, 3H).

Step 3: A mixture of compound C-4 (190 mg, 496 μmol, 1.00 eq), compound C-5 (587 mg, 1.49 mmol, 2.00 mL, 3.00 eq), Pd₂(dba)₃ (45.4 mg, 49.6 μmol, 0.100 eq), SPhos (40.7 mg, 99.2 μmol, 0.200 eq) in DMF (3.00 mL) was degassed and purged with N₂ for 3 times, and then the mixture was stirred at 80° C. for 10 hrs under N₂ atmosphere. The residue was purified by Prep-TLC (EtOAc). TLC (EtOAc, R_{f}=0.23). Compound C-6 (190 mg, crude) was obtained as yellow solid. LC-MS (M+H)⁻ 506.4. ¹H NMR (400 MHz, CDCl₃) δ 8.66-8.63 (m, 1H), 7.95-7.89 (m, 1H), 7.62-7.55 (m, 2H), 7.53-7.42 (m, 2H), 6.56-6.53 (m, 1H), 5.12-5.10 gfr (m, 1H), 4.74-4.71 (m, 1H), 3.67-3.51 (m, 8H), 1.45-1.42 (m, 9H).

Step 4: To a solution of compound C-6 (100 mg, 198 μmol, 1.00 eq) in DCM (1.00 mL) was added HCl/dioxane (4.00 M, 1 mL, 20.2 eq). The mixture was stirred at 25° C. for 2 hrs. The reaction mixture was concentrated under vacuum to give a residue. Compound C-7 (90 mg, crude, HCl) was obtained as yellow solid, it was used into next step directly.

Step 5: To a solution of compound C-7 (80.0 mg, 181 μmol, 1.00 eq, HCl) in pyridine (1.00 mL) was added compound C-8 (34.4 mg, 217 μmol, 1.20 eq) and EDCI (69.4 mg, 362 μmol, 2.00 eq). The mixture was stirred at 25° C. for 2 hrs. The reaction mixture was diluted with saturated NH₄Cl (30 mL), extracted with EtOAc (15 mL*2). The combined organic layers were dried over Na₂SO₄, concentrated under vacuum to give a residue. Compound C-9 (90 mg, crude) was obtained as yellow oil, used for next step directly. LC-MS (M+H)⁺ 546.3.

Step 6: A mixture of compound C-9 (70.0 mg, 128 μmol, 1.00 eq) in H₂O (1.50 mL) was added HCl/dioxane (4.00M, 1.50 mL, 46.8 eq). The mixture was stirred at 60° C. for 4 hrs. The reaction mixture was concentrated under vacuum to give a residue. The residue was purified by Prep-HPLC (column: Waters xbridge 150*25 mm 10 um; mobile phase: [water (10 mM NH₄HCO₃)-ACN]; B %: 5%-35%, 11 min). Compound 210 (62 mg, 97.0% purity) was obtained as white solid. ¹H NMR (400 MHz, DMSO-d₆) δ 8.81-8.75 (m, 1H), 8.56 (dd, J₁=6.0 Hz, J₂=2.0, 1H), 8.16-8.09 (m, 2H), 7.68-7.63 (m, 1H), 7.51-7.43 (m, 3H), 7.10 (t, J=8.0 Hz, 2H), 6.66 (dd, J₁=7.2 Hz, J₂=0.8, 1H), 4.61 (brs, 1H), 3.75-3.67 (m, 1H), 3.55 (d, J=2.0 Hz, 3H), 3.42-3.34 (m, 1H). LC-MS (M+H)⁺ 532.4.

### Example 13: Synthesis of Compound 211

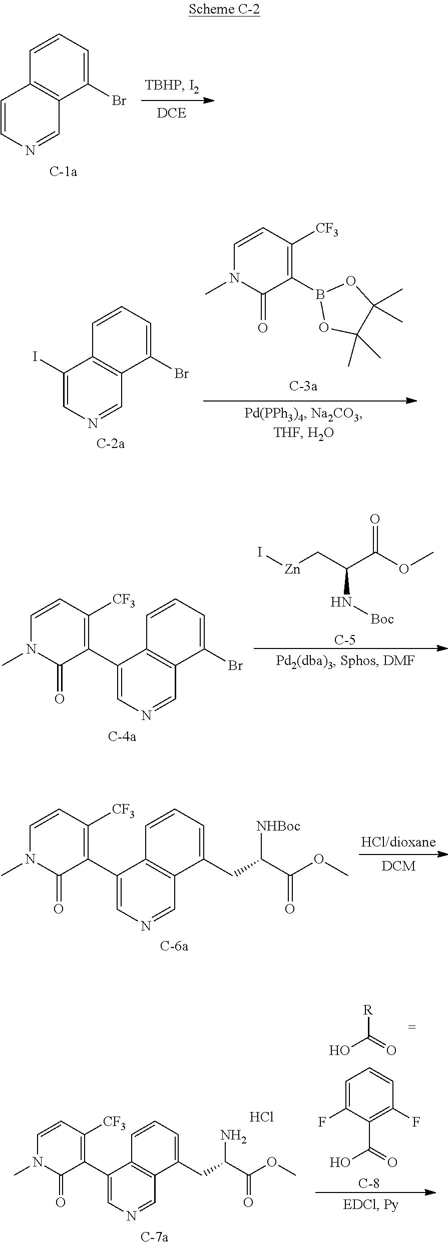

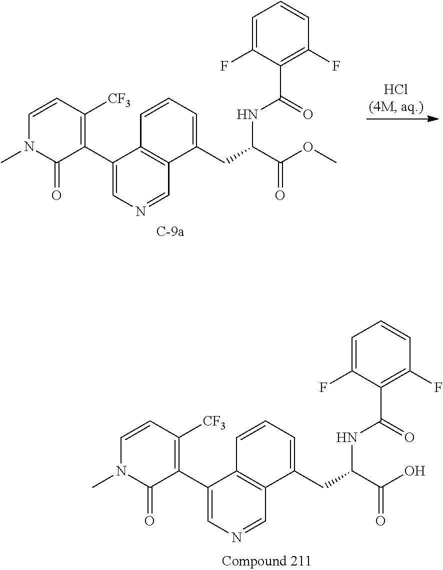

Step 1: To a solution of compound C-1a (2.00 g, 9.61 mmol, 1.00 eq) and I₂ (2.44 g, 9.61 mmol, 1.94 mL, 1.00 eq) in DCE (30 mL) was added TBHP (3.71 g, 28.8 mmol, 3.95 mL, 70.0% purity, 3.00 eq), then the mixture was stirred at 120° C. for 12 hrs. A solution of saturated Na₂SO₃ (50 mL) was added to the mixture, then diluted with H₂O (20 mL) and extracted with DCM (50 mL*3), the combined organic phase was washed with brine (50 mL*2), dried over Na₂SO₄ and concentrated to give a residue. The residue was purified by column chromatography (SiO₂, Petroleum ether:Ethyl acetate=100:1 to 5:1). TLC (Petroleum ether:Ethyl acetate=5:1, R_{f}=0.70). Compound C-2a (2.00 g, 5.99 mmol, 62.3% yield) was obtained as yellow solid. LC-MS: (M+H)⁺: 335.7. H NMR (400 MHz, DMSO-d₆) δ 9.40 (s, 1H), 9.04 (s, 1H), 8.11 (d, J=7.6 Hz, 1H), 8.01 (d, J=8.4 Hz, 1H), 7.82 (t, J=8.0 Hz, 1H).

Step 2: To a solution of compound C-2a (600 mg, 1.80 mmol, 1.00 eq) and compound C-3a (653 mg, 2.16 mmol, 1.20 eq) in THF (6.00 mL) and H₂O (1.50 mL) was added Na₂CO₃ (571 mg, 5.39 mmol, 3.00 eq) and Pd(PPh₃) 4 (208 mg, 180 μmol, 0.100 eq) under N₂, then the mixture was stirred at 65° C. for 2 hrs. The mixture was diluted with H₂O (20 mL) and extracted with ethyl acetate (20 mL*3), the combined organic phase was washed with brine (50 mL*2), dried over Na 2SO₄ and concentrated to give a residue. The residue was purified by Prep-TLC (Petroleum ether:Ethyl acetate=0:1, R_{f}=0.25). Compound C-4a (70.0 mg, 183 μmol, 10.2% yield) was obtained as yellow solid. LC-MS (M+H)⁺ 383.0. ¹H NMR (400 MHz, DMSO-d₆) δ 9.54 (s, 1H), 8.41 (s, 1H), 8.17 (d, J=7.2 Hz, 1H), 8.04 (d, J=7.2 Hz, 1H), 7.64 (t, J=8.0 Hz, 1H), 7.53 (d, J=8.4 Hz, 1H), 6.71 (d, J=7.2 Hz, 1H), 3.56 (s, 3H).

Step 3: To a solution of compound C-4a (120 mg, 313 μmol, 1.00 eq), Pd₂(dba)₃ (28.7 mg, 31.3 μmol, 0.100 eq), SPhos (12.9 mg, 31.3 μmol, 0.100 eq) in DMF (2.00 mL) was added compound C-5 (371 mg, 940 μmol, 3.00 eq) in DMF (2.00 mL) under N₂, then the mixture was stirred at 80° C. for 2 hrs. The mixture was diluted with H₂O (20 mL) and extracted with ethyl acetate (20 mL*3), the combined organic phase was washed with brine (30 mL*3), dried over Na₂SO₄ and concentrated to give a residue. The residue was purified by Prep-TLC (Petroleum ether:Ethyl acetate=0:1, R_{f}=0.30). Compound C-6a (100 mg, crude) was obtained as yellow solid. LC-MS: (M+H)⁺: 309.4. ¹H NMR (400 MHz, DMSO-d₆) δ 9.53 (d, J=8.0 Hz, 1H), 8.28 (s, 1H), 8.15-8.13 (m, 1H), 7.62-7.60 (m, 1H), 7.54-7.50 (m, 2H), 7.33 (d, J=8.4 Hz, 1H), 6.70-6.67 (m, 1H), 4.39-4.38 (m, 1H), 3.69 (dd, J₁=14.8 Hz, J₂=4.8 Hz, 1H), 3.60-3.55 (m, 6H), 3.51-3.43 (m, 1H), 1.29 (d, J=2.0 Hz, 9H).

Step 4: To a solution of compound C-6a (100 mg, 198 μmol, 1.00 eq) in DCM (1.00 mL) was added HCl/dioxane (4.00 M, 1.00 mL, 20.2 eq) at 0° C., then the mixture was stirred at 25° C. for 2 hrs. The mixture was concentrated to give a residue. Compound C-7a (80.0 mg, crude, HCl) was obtained as yellow solid. LC-MS (M+H)⁺ 406.2.

Step 5: To a solution of compound C-7a (70.0 mg, 158 μmol, 1.00 eq, HCl) and compound C-8 (25.1 mg, 158 μmol, 1.00 eq) in pyridine (1.00 mL) was added EDCI (60.7 mg, 317 μmol, 2.00 eq), then the mixture was stirred at 25° C. for 1 hr. The mixture was diluted with H₂O (20 mL) and extracted with ethyl acetate (20 mL*3), the combined organic phase was dried over Na₂SO₄ and concentrated to give a residue. Compound C-9a (70.0 mg, crude) was obtained as yellow solid. LC-MS (M+H)⁺ 686.2.

Step 6: A solution of compound C-9a (50.0 mg, 91.7 μmol, 1.00 eq) in aqueous HCl (4.00 M, 0.500 mL, 21.8 eq) was stirred at 60° C. for 2 hrs. The residue was concentrated to give a residue. The residue was purified by Prep-HPLC (column: Waters xbridge 150*25 mm 10 um; mobile phase: [water (10 mM NH₄HCO₃)-ACN]; B %: 7%-37%, 11 min). Compound 211 (15.09 mg, 28.2 μmol, 30.8% yield, 99.4% purity) was obtained as white solid. LC-MS (M+H)⁺ 532.2. ¹H NMR (400 MHz, CD₃CN) δ 9.75-9.68 (m, 1H), 8.21-8.18 (m, 1H), 7.80-7.78 (m, 1H), 7.58-7.52 (m, 2H), 7.39-7.37 (m, 2H), 6.95-6.93 (m, 2H), 6.60 (t, J=6.4 Hz, 1H), 4.95-4.90 (m, 1H), 3.90 (dd, J₁=14.0 Hz, J₂=5.2 Hz, 1H), 3.69 (dd, J₁=14.4 Hz, J₂=6.4 Hz, 1H), 3.52-3.49 (m, 3H).

### Example 14: Synthesis of Compound 212

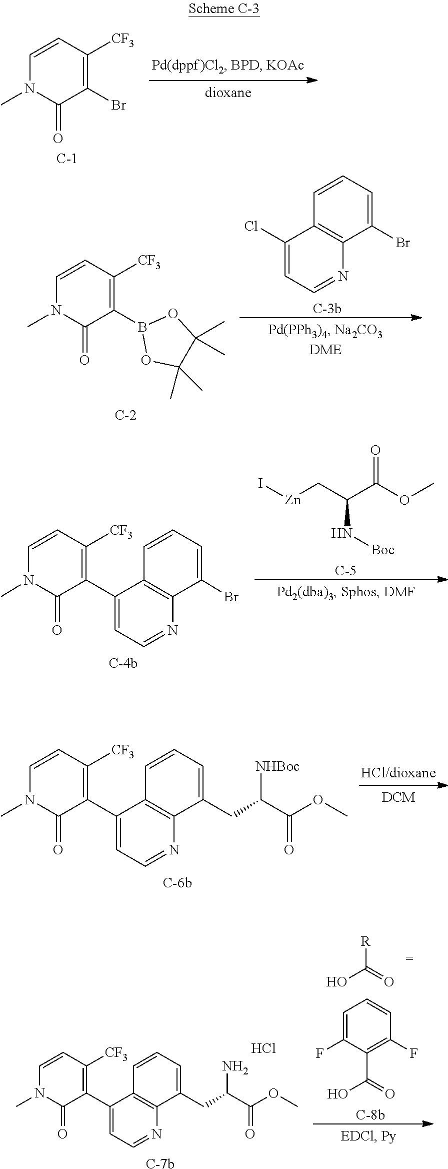

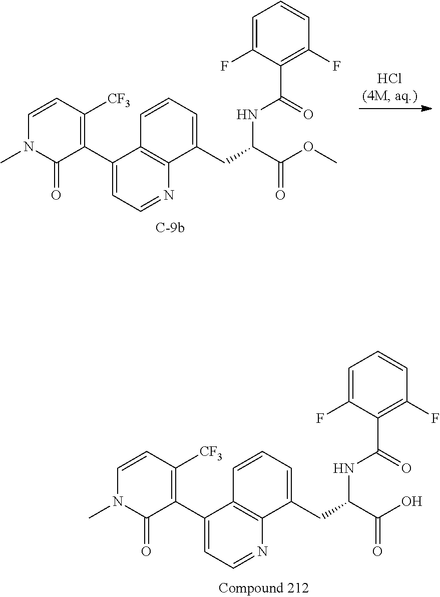

Step 1: A mixture of compound C-1 (4.50 g, 17.6 mmol, 1.00 eq), BPD (22.3 g, 87.9 mmol, 5.00 eq), Pd(dppf)Cl₂ (2.57 g, 3.52 mmol, 0.200 eq), KOAc (10.4 g, 105 mmol, 6.00 eq) and in dioxane (80 mL) was degassed and purged with N₂ for 3 times, and then the mixture was stirred at 90° C. for 12 hrs under N₂ atmosphere. The reaction mixture was filtered and concentrated under reduced pressure to give a residue. The residue was purified by column chromatography (SiO₂, Petroleum ether:Ethyl acetate=1:0 to 0:1). Compound C-2 (7.00 g, crude) was obtained as a white solid. LC-MS (M+H)⁺ 303.9. ¹H NMR (400 MHz, CDCl₃) δ 7.43 (d, J=7.2 Hz, 1H), 6.87 (s, 1H), 3.58 (s, 1H), 1.27-1.21 (m, 12H).

Step 2: A mixture of compound C-3b (500 mg, 1.74 mmol, 1.00 eq), compound 2 (480 mg, 1.58 mmol, 0.908 eq), Na₂CO₃ (2.00 M, 2.61 mL, 3.00 eq), Pd(PPh₃)⁺ (201 mg, 174 μmol, 0.100 eq) in DME (5 mL) was degassed and purged with N₂ for 3 times, and then the mixture was stirred at 90° C. for 3 hrs under N₂ atmosphere. The reaction mixture was partitioned between ethyl acetate 50 mL*3 and H₂O 50 mL. The organic phase was separated, washed with brine (50 mL*3), dried over Na₂SO₄, filtered and concentrated under reduced pressure to give a residue. The residue was purified by column chromatography (SiO₂, Petroleum ether:Ethyl acetate=100:1 to 0:1). Compound C-4b (80.0 mg, 0.209 mmol, 12.0% yield) was obtained as yellow solid. LC-MS (M+H)⁻ 385.1. ¹H NMR (400 MHz, DMSO-d₆) δ 9.06 (d, J=4.4 Hz, 1H), 8.17 (t, J=6.0 Hz, 2H), 7.54 (d, J=8.4 Hz, 1H), 7.48-7.43 (m, 2H), 6.72 (d, J=7.2 Hz, 1H), 3.56 (s, 3H).

Step 3: To a solution of compound C-4b (80.0 mg, 209 μmol, 1.00 eq), Pd₂ (dba); (19.1 mg, 20.9 μmol, 0.100 eq) and SPhos (17.1 mg, 41.8 μmol, 0.200 eq) in DMF (2.00 mL) was added compound C-5 (247 mg, 626 μmol, 3.00 eq), then the mixture was stirred at 80° C. for 12 hrs. The reaction mixture was quenched by addition H₂O 4.00 mL at 0° C., and then diluted with H₂O 10 mL and extracted with ethyl acetate (10 mL*3). The combined organic layers were washed with brine (10 mL*3), dried over Na₂SO₄ filtered and concentrated under reduced pressure to give a residue. The product was purified by Prep-TLC (Petroleum ether:Ethyl acetate=0.1, R_{f}=0.30). Compound C-6b (90.0 mg, 178 μmol, 85.3% yield) was obtained as yellow solid. LC-MS (M+H)⁻ 506.2. ¹H NMR (400 MHz, CDCl₃) δ 8.98 (s, 1H), 7.68-7.56 (m, 2H), 7.47-7.38 (m, 2H), 6.58 (d, J=7.2 Hz, 1H), 6.31-6.27 (m, 1H), 4.65-4.63 (m, 1H), 3.84-3.78 (m, 1H), 3.67-3.64 (m, 7H), 1.38-1.29 (m, 9H).

Step 4: To a solution of compound C-6b (80.0 mg, 158 μmol, 1.00 eq) in HCl/dioxane (2.00 mL) and DCM (2.00 mL). The mixture was stirred at 25° C. for 2 hr. The mixture was concentrated to give a residue. Compound C-7b (60.0 mg, crude, HCl) was obtained as yellow solid and used into the next step without further purification. LC-MS (M+H)⁻ 406.1.

Step 5: To a solution of compound C-7b (50.0 mg, 113 μmol, 1.00 eq, HCl) and compound C-8b (21.5 mg, 136 μmol, 1.20 eq) in pyridine (2.00 mL) was added EDCI (43.4 mg, 226 μmol, 2.00 eq). The mixture was stirred at 25° C. for 1 hr. The mixture was concentrated to give a residue. Compound C-9b (50.0 mg, crude) was obtained as yellow solid and used into the next step without further purification. LC-MS (M+H)⁻ 546.2.

Step 6: A solution of compound C-9b (60.0 mg, 110 μmol, 1.00 eq) in aqueous HCl (4.00 M, 27.5 uL, 1.00 eq) was stirred at 60° C. for 2 hrs. The mixture was concentrated to give a residue.

The residue was purified by prep-HPLC (column: Waters xbridge 150*25 mm 10 um; mobile phase: [water (10 mM NH₄HCO₃)-ACN]; B %: 7.00%-37.0%, 11 min). Compound 212 (24.35 mg, 44.5 μmol, 40.5% yield, 97.2% purity) was obtained as off-white solid. LC-MS (M+H)⁺ 530.1. ¹H NMR (400 MHz, DMSO-d₆) δ 8.96-8.94 (m, 1H), 8.72 (t, J=9.2 Hz 1H), 8.15 (d, J=7.2 Hz 1H), 7.69 (t, J=8.0 Hz, 1H), 7.43-7.40 (m, 2H), 7.34-7.32 (m, 2H), 7.09-7.05 (m, 2H), 6.68 (d, J=6.4 Hz, 1H), 4.77-4.71 (m, 1H), 4.02-3.98 (m, 1H), 3.55 (s, 3H), 3.42-3.33 (m, 1H).

The following compounds, set forth in Table 8, were also prepared using the synthetic procedures of Scheme C-3, or analogous procedures thereto:

TABLE 8

| Compound | |
|---|---|
| No. | Characterization Data |
| 213 | 654.1 [M + 1] |
| 214 | 685.2 [M + 1] |
| 215 | 681.2 [M + 1] |
| 216 | 631.4 [M + 1] |
| 217 | 643.4 [M + 1] |
| 218 | 685.2 [M + 1] |
| 221 | 780.2 [M + 1] |
| 224 | 816.1 [M + 1] |
| 225 | 782.2 [M + 1] |
| 226 | 800.2 [M + 1] |
| 228 | 630.0 [M + 1] |
| 230 | 590.1 [M + 1] |
| 232 | 616.8 [M + 1] |
| 237 | 711.2 [M + 1] |
| 245 | 766.5 [M + 1] |
| 246 | 631.3 [M + 1] |
| 248 | 779.6 [M + 1] |
| 249 | 698.4 [M + 1] |
| 250 | 710.2 [M + 1] |
| 252 | 776.2 [M + 1] |
| 253 | 750.4 [M + 1] |
| 254 | 694.2 [M + 1] |
| 257 | 657.3 [M + 1] |
| 258 | 582.0 [M + 1] |
| 261 | 646.1 [M + 1] |
| 263 | 614.2 [M + 1] |

### Example 15: Synthesis of Compound 231

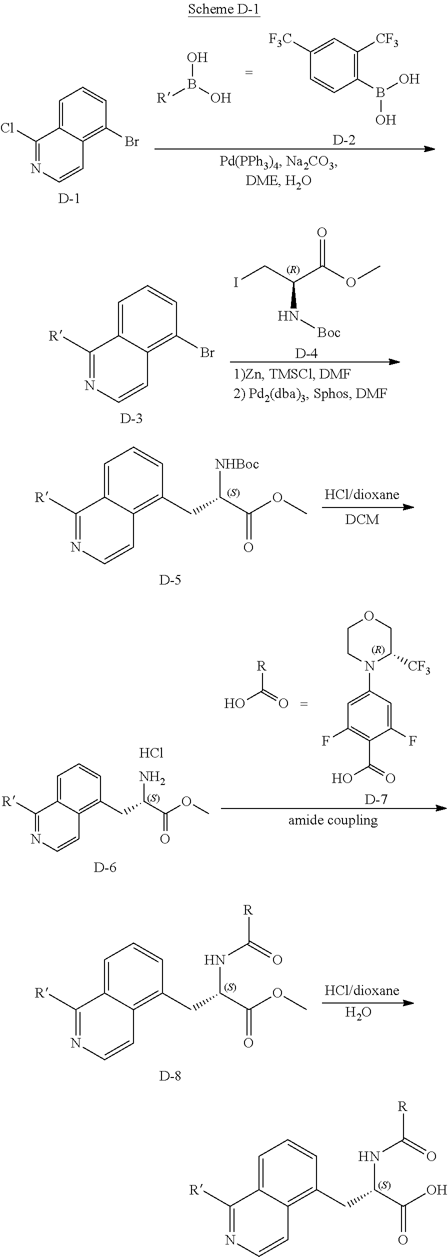

Step 1: A mixture of compound D-2 (2.00 g, 7.75 mmol, 1.00 eq), compound D-1 (2.26 g, 9.31 mmol, 1.20 eq), Pd(PPh₃)₄ (1.79 g, 1.55 mmol, 0.200 eq), Na₂CO₃ (1.64 g, 15.5 mmol, 2.00 eq) and in DME (20 mL) and H₂O (10 mL) was degassed and purged with N₂ for 3 times, and then the mixture was stirred at 90° C. for 12 hrs under N₂ atmosphere. The mixture was concentrated under reduced pressure to give a residue. The residue was purified by column chromatography (SiO₂, Petroleum ether/Ethyl acetate=1/0 to 1/1). Compound D-3 (1.50 g, 3.57 mmol, 46.0% yield) was obtained as white solid. ¹H NMR (400 MHz, DMSO-d₆) δ 8.75 (d, J=5.6 Hz, 1H), 8.30 (s, 1H), 8.27-8.20 (m, 2H), 8.10 (d, J=6.0 Hz, 1H), 7.85 (d, J=8.0 Hz, 1H), 7.56-7.50 (m, 2H).

Step 2: To a solution of compound D-3 (500 mg, 1.19 mmol, 1.00 eq), SPhos (97.7 mg, 238 μmol, 0.200 eq) and Pd₂(dba)₃ (109 mg, 119 μmol, 0.100 eq) in DMF (5 mL) was added compound D-4 (1.41 g, 3.57 mmol, 3.00 eq), then the mixture was stirred at 80° C. for 12 hrs under N₂ atmosphere. The residue was diluted with H₂O (50 mL) and extracted with ethyl acetate (30 mL*2). The combined organic layers were washed with brine (30 mL*2), dried over Na 2SO₄, filtered and concentrated under reduced pressure to give a residue. The residue was purified by column chromatography (SiO₂, Petroleum ether/Ethyl acetate=100/1 to 1/1). TLC (Petroleum ether/Ethyl acetate=5/1). Compound D-5 (0.500 g, 832 μmol, 69.9% yield, 90.3% purity) was obtained as yellow oil. LC-MS (M+H)⁺ 543.2.

Step 3: To a solution of compound D-5 (0.500 g, 832 μmol, 90.3% purity, 1.00 eq) in DCM (5 mL) was added HCl/dioxane (4.00 M, 2.00 mL, 9.61 eq) at 0° C., then the mixture was stirred at 15° C. for 2 hrs. The mixture was concentrated under reduced pressure to give a residue. Compound D-6 (0.39 g, 814.51 μmol, 97.86% yield, HCl) was obtained as yellow solid. LC-MS (M+H)⁺ 443.2.

Step 4: To a solution of compound D-6 (60.0 mg, 125 μmol, 1.00 eq, HCl) and compound D-7 (42.9 mg, 138 μmol, 1.10 eq) in pyridine (2.00 mL) was added EDCI (48.0 mg, 251 μmol, 2.00 eq), then the mixture was stirred at 15° C. for 12 hrs. The residue was diluted with sat. NaHCO₃ aq. (30 mL) and extracted with ethyl acetate (15 mL*3). The combined organic layers were washed with brine (15 mL*3), dried over Na₂SO₄, filtered and concentrated under reduced pressure to give a residue. The residue was purified by prep-TLC (SiO₂, DCM/MeOH=10/1). Compound D-8 (90.0 mg, 111 μmol, 88.4% yield, 90.5% purity) was obtained as white solid. LC-MS (M+H)⁻ 736.0.

Step 5: To a solution of compound D-8 (80.0 mg, 98.4 μmol, 90.5% purity, 1.00 eq) in H₂O (1.00 mL) was added HCl/dioxane (4.00 M, 4.00 mL, 163 eq), then the mixture was stirred at 60° C. for 12 hrs. The mixture was concentrated under reduced pressure to give a residue. The residue was purified by prep-HPLC (column: Phenomenex Luna C18 100*30 mm*5 um; mobile phase: [water (FA)-ACN]; B %: 46%-76%, 8 min). The residue was purified by prep-HPLC (column: Waters xbridge 150*25 mm 10 um; mobile phase: [water (NH₄HCO₃)-ACN]; B %: 27%-57%, 8 min). Compound 231 (35.92 mg, 49.7 μmol, 50.5% yield, 99.8% purity) was obtained as white solid. LC-MS (M+H)⁺: 722.2. ¹H NMR (400 MHz, CD₃CN) δ 8.60 (t, J=6.4 Hz, 1H), 8.21 (s, 1H), 8.12 (t, J=6.0 Hz, 1H), 8.07 (brd, J=7.6 Hz, 1H), 7.70-7.64 (m, 2H), 7.50-7.44 (m, 1H), 7.42-7.36 (m, 1H), 7.21-7.13 (m, 1H), 6.52 (d, J=11.2 Hz, 2H), 5.01-4.91 (m, 1H), 4.45-4.34 (m, 1H), 4.22 (d, J=12.8 Hz, 1H), 4.01-3.93 (m, 1H), 3.88-3.72 (m, 2H), 3.64-3.45 (m, 2H), 3.40-3.30 (m, 2H).

The following compounds, set forth in Table 9, were also prepared using the synthetic procedures of Scheme D-1, or analogous procedures thereto:

TABLE 9

| Compound | |
|---|---|
| No. | Characterization Data |
| 219 | 718.2 [M + 1] |
| 220 | 637.0 [M + 1] |
| 222 | 728.3 [M + 1] |
| 255 | 702.4 [M + 1] |
| 256 | 646.1 [M + 1] |
| 259 | 704.1 [M + 1] |
| 260 | 551.2 [M + 1] |
| 275 | 569.2 [M + 1] |

### Example 16: Synthesis of Compound 268

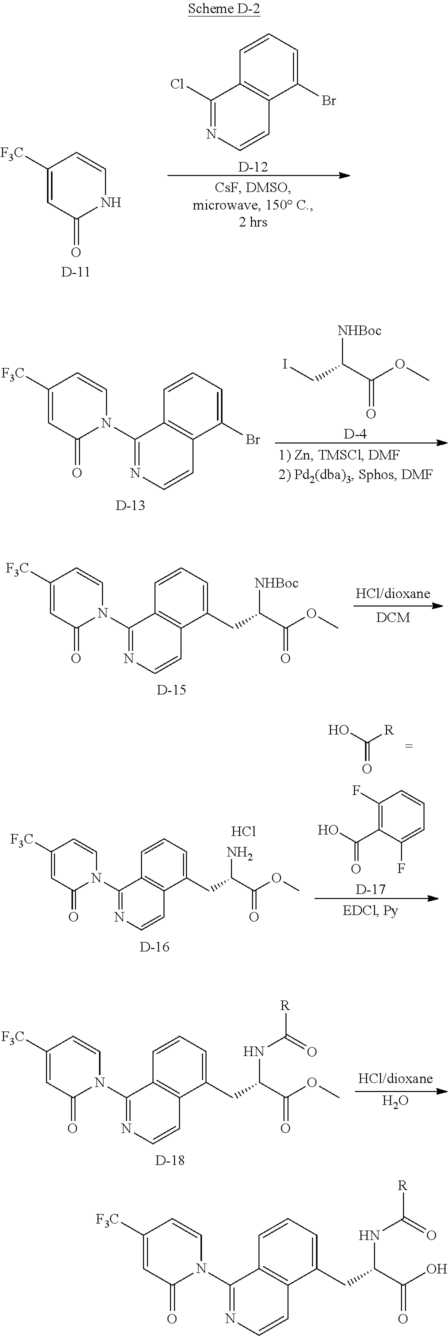

Step 1: Compound D-11 (1.00 g, 6.13 mmol, 1.00 eq), Compound D-12 (1.93 g, 7.97 mmol, 1.30 eq) and CsF (2.79 g, 18.3 mmol, 678 μL, 3.00 eq) in DMSO (10 mL) were added into a microwave tube. The sealed tube was heated at 140° C. for 1 hr under microwave. The residue was diluted with H₂O (20 mL) and extracted with EtOAc (20 mL*3). The combined organic layers were washed with water (20 mL*2), filtered and concentrated under reduced pressure to give a residue. The residue was purified by column chromatography (SiO₂, Petroleum ether:Ethyl acetate=5:1) (Petroleum ether:Ethyl acetate=5:1, R_{f}=0.35). Compound D-13 (923 mg, 2.50 mmol, 40.7% yield) was obtained as white solid. LC-MS (M+H)⁺ 370.9. ¹H NMR (400 MHz, CDCl₃) δ 8.63 (d, J=6.0 Hz, 1H), 8.24 (d, J=6.0 Hz, 1H), 8.06 (dd, J₁=7.2 Hz, J₂=0.4 Hz, 1H), 7.69 (d, J=8.4 Hz, 1H), 8.24 (d, J=7.6 Hz, 1H), 7.51 (t, J=7.6 Hz, 1H), 7.02 (s, 1H), 6.51 (dd, J₁=7.6 Hz, J₂=2.0 Hz, 1H).

Step 2: Zinc (4.77 g, 72.9 mmol, 3.00 eq) was added to a three necked flask, and heated at 110° C. under vacuum for 10 minutes, then cooled to 25° C. To the flask was added a solution of TMSCl (528 mg, 4.86 mmol, 616 μL, 0.20 eq) in DMF (20 mL) and the inner temperature was gradually rose to 30° C. The mixture was stirred at 25° C.-30° C. for 20 minutes. And the supernatant liquor was removed by syringe. Then a mixture of methyl compound D-4 (8.00 g, 24.3 mmol, 1.00 eq) and TMSCl (528 mg, 4.86 mmol, 616 uL, 0.20 eq) in DMF (30 mL) was added to the precipitate, and the inner temperature was rapidly rose from 25° C. to 45° C. And the resulting mixture was stirred at 25° C. for 1 hr under N₂. Compound [(2R)-2-(tert-butoxycarbonylamino)-3-methoxy-3-oxo-propyl]-iodo-zinc (9.59 g, crude) in DMF (30 mL) was obtained as a gray liquid, and used next step directly. To a solution of compound 3 (500 mg, 1.35 mmol, 1.00 eq) and [(2R)-2-(tert-butoxycarbonylamino)-3-methoxy-3-oxo-propyl]-iodo-zinc (1.60 g, 4.06 mmol, 3.00 eq) in DMF (10 mL) was added Pd₂(dba)₃ (124 mg, 135 μmol, 0.10 eq) and SPhos (111 mg, 270 μmol, 0.20 eq) under N₂. The mixture was stirred at 85° C. for 12 hrs. The reaction mixture was filtered and concentrated under reduced pressure to give a residue. The residue was purified by column chromatography (SiO₂, Petroleum ether:Ethyl acetate=10:1), (Dichloromethane:Methanol=10:1, R_{f}=0.33). Compound D-15 (340 mg, 635 μmol, 46.9% yield, 91.9% purity) was obtained as yellow oil. LC-MS (M-55)⁺ 435.9. ¹H NMR (400 MHz, CDCl₃) δ 8.59-8.57 (m, 1H), 8.13-8.09 (t, J=6.0 Hz, 1H), 7.63-7.54 (m, 4H), 7.02 (s, 1H), 6.52-6.49 (m, 1H), 5.15 (brs, 1H), 4.74-4.69 (m, 1H), 3.73-3.63 (m, 4H), 3.60 (d, J=5.6 Hz, 1H), 1.42 (d, J=15.2 Hz, 9H).

Step 3: To a solution of compound D-15 (340 mg, 635 μmol, 91.9% purity, 1.00 eq) in DCM (4.00 mL) was added HCl/dioxane (4.00 M, 3.00 mL, 18.8 eq) at 0° C. The mixture was stirred at 25° C. for 12 hrs. The reaction mixture was concentrated under reduced pressure to give a residue. A slurry was formed with crude product and DCM (20 mL) at 20° C. for 30 min. The mixture was filtered and the filter cake concentrated under reduced pressure to give a residue. Compound D-26 (200 mg, 440 μmol, 69.3% yield, 94.3% purity, HCl) was obtained as yellow solid. LC-MS: (M+H)⁺: 392.1.

Step 4: To a solution of compound D-16 (80.0 mg, 176 μmol, 94.3% purity, 1.00 eq, HCl) and compound D-17 (30.6 mg, 193 μmol, 1.10 eq) in pyridine (1.00 mL) was added EDCI (101 mg, 529 μmol, 3.00 eq). The mixture was stirred at 25° C. for 1.5 hrs. The reaction mixture was concentrated under reduced pressure to remove pyridine. The residue was diluted with sat.aq. NaHCO₃ (20 mL) and extracted with EtOAc (20 mL*3). The combined organic layers were washed with sat.aq. NH₄Cl (20 mL*2) and brine (20 mL*2), dried over Na₂SO₄, filtered and concentrated under reduced pressure to give a residue. The residue was purified by Prep-TLC (Dichloromethane:Methanol=10:1, R_{f}=0.44). Compound D-18 (55.0 mg, 97.9 μmol, 55.5% yield, 94.6% purity) was obtained as white solid. LC-MS (M+H)⁻ 532.0. ¹H NMR (400 MHz, CDCl₃) δ 8.58 (dd, J₁=13.6 Hz, J₂=5.6 Hz, 1H), 8.23 (dd, J₁=18.4 Hz, J₂=5.6 Hz, 1H), 7.64-7.53 (m, 4H), 7.42-7.38 (m, 1H), 7.01-6.94 (m, 3H), 6.69 (t, J=18.0 Hz, 1H), 6.50 (d, J=7.6 Hz, 1H), 5.25-5.21 (m, 1H), 3.79-3.66 (m, 5H).

Step 5: To a solution of compound D-18 (50.0 mg, 89.0 μmol, 94.6% purity, 1.00 eq) in H₂O (1.00 mL) was added HCl/dioxane (4.00 M, 2.00 mL, 89.8 eq). The mixture was stirred at 60° C. for 2 hrs. The reaction mixture was concentrated under reduced pressure to remove water and give a residue. The residue was purified by Prep-HPLC (column: Welch Ultimate AQ-C18 150*30 mm*5 um; mobile phase: [water (HCl)-ACN]; B %: 28%-58%, 10 min). Compound 268 (30.95 mg, 58.6 μmol, 65.9% yield, 98.1% purity) was obtained as white solid. LC-MS (M+H)⁺ 517.9. ¹H NMR (400 MHz, DMSO-d₆) δ 13.1 (brs, 1H), 9.28 (t, J=16.4 Hz, 1H), 8.62 (dd, J₁=11.6 Hz, J₂=5.6 Hz, 1H), 8.24 (d, J=6.0 Hz, 1H), 8.04 (dd, J₁=38.0 Hz, J₂=7.2 Hz, 1H), 7.78 (d, J=7.2 Hz, 1H), 7.67-7.58 (m, 2H), 7.54-7.44 (m, 1H), 7.13 (t, J=8.0 Hz, 2H), 7.03 (d, J=13.6 Hz, 1H), 6.69 (dd, J₁=7.2 Hz, J₂=2.0 Hz, 1H), 4.79-4.71 (m, 1H), 3.84-3.71 (m, 1H), 3.48-3.40 (m, 1H).

### Example 17: Synthesis of Compounds 281 and 282

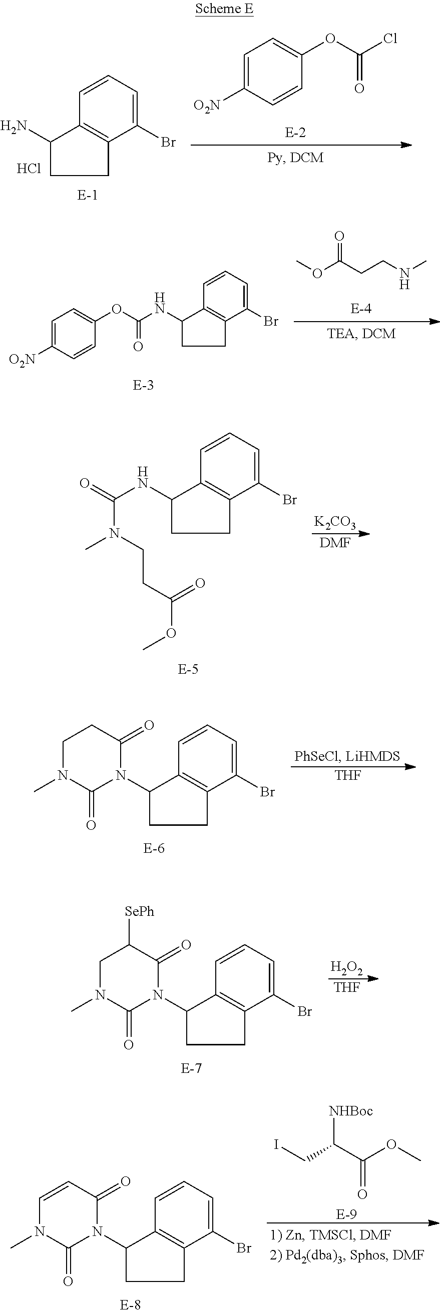

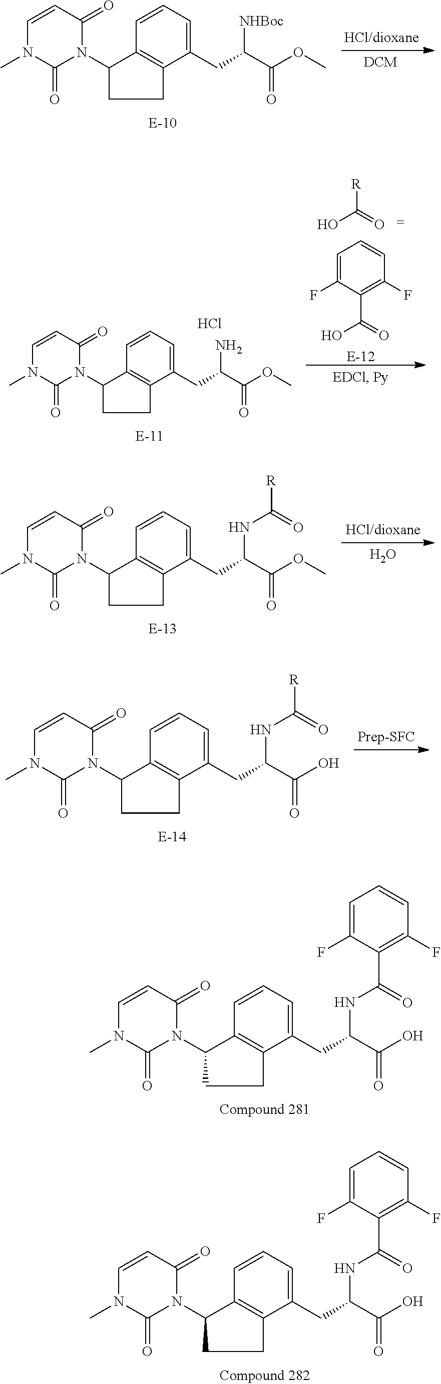

Step 1: To the mixture of compound E-1 (3.70 g, 14.9 mmol, 1.00 eq, HCl) and pyridine (2.94 g, 37.2 mmol, 3.00 mL, 2.50 eq) in DCM (40 mL) was added compound E-2 (3.90 g, 19.4 mmol, 1.30 eq) at 0° C., then the mixture of 25° C. for 2 hrs. Compound E-3 (5.62 g, crude) was obtained as yellow liquid in DCM (40 mL), which was used to next step.

Step 2: The mixture of compound E-3 (5.62 g, 14.9 mmol, 1.00 eq) and TEA (3.02 g, 29.8 mmol, 4.15 mL, 2.00 eq) was added a solution of compound E-4 (2.62 g, 22.4 mmol, 1.50 eq) in DCM (20 mL), the mixture was stirred at 25° C. for 1 hr. The residue was diluted with HCl (1.00M, 50 mL) and extracted with DCM (30 mL*3). The combined organic layers were dried over Na 2SO₄, filtered and concentrated under reduced pressure to give a residue. The residue was purified by column chromatography (SiO₂, Petroleum ether/Ethyl acetate=300/1 to 1/5). TLC (Petroleum ether/Ethyl acetate=1/1, I₂). Compound E-5 (4.50 g, 12.7 mmol, 85.0% yield) was obtained as purple oil. LC-MS (M+H)⁻ 354.9. ¹H NMR (400 MHz, CDCl₃) δ 7.39 (d, J=7.6 Hz, 1H), 7.29 (s, 1H), 7.09 (t, J=8.0 Hz, 1H), 5.43 (q, J=8.0 Hz, 1H), 5.25 (br d, J=7.6 Hz, 1H), 3.68 (s, 3H), 3.64-3.50 (m, 2H), 3.09-2.97 (m, 1H), 2.91 (s, 3H), 2.89-2.78 (m, 1H), 2.70-2.57 (m, 3H), 1.89-1.75 (m, 1H).

Step 3: To a solution of compound E-5 (3.50 g, 9.85 mmol, 1.00 eq) in DMF (35 mL) was added K₂CO₃ (5.45 g, 39.4 mmol, 4.00 eq) at 25° C., then the mixture was stirred at 80° C. for 4 hrs. The reaction mixture was quenched by addition to 1.00 M HCl 150 mL at 0° C., and then diluted with H₂O (20 mL) and extracted with DCM (50 mL*2). The combined organic layers were washed with H₂O (50 mL*5), dried over Na₂SO₄, filtered and concentrated under reduced pressure to give a residue. Compound E-6 (4.00 g) was obtained as yellow oil. LC-MS (M+H)⁻ 324.9. ¹H NMR (400 MHz, CDCl₃) δ 7.34 (d, J=7.6 Hz, 1H), 7.04-6.93 (m, 2H), 6.30 (dd, J₁=9.6 Hz, J₂=6.8 Hz, 1H), 3.44-3.37 (m, 2H), 3.31-3.21 (m, 1H), 3.03 (s, 3H), 2.97-2.85 (m, 1H), 2.76-2.71 (m, 2H), 2.52-2.41 (m, 1H), 2.38-2.27 (m, 1H).

Step 4: To a solution of compound E-6 (3.00 g, 9.28 mmol, 1.00 eq) and PhSeCl (1.78 g, 9.28 mmol, 1.00 eq) in THF (30 mL) was added drop-wise LiHMDS (1.00 M, 13.9 mL, 1.50 eq) at −78° C., then the mixture was stirred at −78° C. for 2 hrs. The reaction mixture was quenched by HCl (1.00M, 50 mL) at 0° C. and extracted with DCM (30 mL*3). The combined organic layers were dried over Na₂SO₄, filtered and concentrated under reduced pressure to give a residue. The residue was purified by column chromatography (SiO₂, Petroleum ether/Ethyl acetate=300/1 to 1/5). TLC (Petroleum ether/Ethyl acetate=3/1). Compound E-6 (1.30 g, 4.02 mmol, 43.3% yield) was recycled as yellow oil. Compound E-7 (2.50 g, 5.04 mmol, 54.3% yield, 96.4% purity) was obtained as yellow solid. LC-MS (M+H)⁻ 478.9. ¹H NMR (400 MHz, CDCl₃) δ 7.71-7.65 (m, 1H), 7.52 (d, J=6.8 Hz, 1H), 7.42-7.27 (m, 4H), 7.11-6.98 (m, 2H), 6.34-6.23 (m, 1H), 4.03-3.85 (m, 2H), 3.45-3.35 (m, 1H), 3.32-3.19 (m, 1H), 3.00 (d, J=9.2 Hz, 3H), 2.96-2.85 (m, 1H), 2.50-2.37 (m, 1H), 2.37-2.20 (m, 1H).

Step 5: To a solution compound E-7 (2.50 g, 5.04 mmol, 96.4% purity, 1.00 eq) in THF (25 mL) was added H₂O₂ (4.74 g, 41.8 mmol, 4.02 mL, 30% purity, 8.30 eq) at 0° C., the mixture was stirred at 25° C. for 10 min. The reaction mixture was quenched by addition sat.aq Na₂SO₃ (30 mL) at 0° C., extracted with DCM (30 mL*3). The combined organic layers were washed with H₂O (30 mL*3), dried over Na₂SO₄, filtered and concentrated under reduced pressure to give a residue. Compound E-8 (1.62 g, 4.82 mmol, 95.6% yield, 95.5% purity) was obtained as yellow solid. ¹H NMR (400 MHz, CDCl₃) δ 7.37 (d, J=7.6 Hz, 1H), 7.13 (d, J=8.0 Hz, 1H), 7.05-6.94 (m, 2H), 6.62 (t, J=8.0 Hz, 1H), 5.74 (brd, J=6.4 Hz, 1H), 3.47-3.17 (m, 4H), 3.05-2.89 (m, 1H), 2.59-2.32 (m, 2H). LC-MS (M+H)⁻ 322.9.

Step 6: To a solution of compound E-8 (1.62 g, 4.82 mmol, 95.5% purity, 1.00 eq), Pd₂ (dba); (441 mg, 482 μmol, 0.100 eq), and SPhos (198 mg, 482 μmol, 0.100 eq) in DMF (10 mL) was added compound E-9 (5.70 g, 14.5 mmol, 3.00 eq) at 25° C., the mixture was stirred at 90° C. for 12 hrs under N₂ atmosphere. The reaction mixture was filtered. The filter was diluted with H₂O (50 mL) and extracted with ethyl acetate (50 mL*2). The combined organic layers were washed with brine (50 mL*2), dried over Na₂SO₄, filtered and concentrated under reduced pressure to give a residue. The residue was purified by column chromatography (SiO₂, Petroleum ether/Ethyl acetate-300/1 to 1/5). TLC (Petroleum ether/Ethyl acetate=1/1). Compound E-10 (400 mg, 753 μmol, 15.6% yield, 83.5% purity) was obtained as yellow oil. LC-MS (M+H)⁻ 444.1.

Step 7: To a solution of compound E-10 (400 mg, 753 μmol, 83.5% purity, 1.00 eq) in DCM (3.00 mL) was added HCl/dioxane (4.00 M, 1 mL, 5.31 eq) at 0° C., then the mixture was stirred at 25° C. for 1 hr. The mixture was concentrated under reduced pressure to give a residue. Compound E-11 (0.34 g, crude, HCl) was obtained as yellow solid. LC-MS (M+H)⁺ 344.1.

Step 8: To a solution of compound E-11 (340 mg, 8952 μmol, 1.00 eq, HCl) and compound E-12 (313 mg, 1.98 mmol, 2.21 eq) in pyridine (5 mL) was added EDCI (380 mg, 1.98 mmol, 2.21 eq), then the mixture was stirred at 25° C. for 2 hrs. The residue was diluted with sat.aq. NaHCO₃ (20 mL) and extracted with ethyl acetate (15 mL*3). The combined organic layers were washed with brine (15 mL*3), dried over Na₂SO₄, filtered and concentrated under reduced pressure to give a residue. The residue was purified by prep-TLC (SiO₂, DCM:MeOH=10:1). Compound E-13 (270 mg, 533 μmol, 59.5% yield, 95.4% purity) was obtained as white solid. LC-MS (M+H)⁻ 484.2. ¹H NMR (400 MHz, CDCl₃) δ 7.42-7.30 (m, 1H), 7.16-6.98 (m, 3H), 6.98-6.89 (m, 3H), 6.69-6.49 (m, 2H), 5.78-5.58 (m, 1H), 5.19-5.07 (m, 1H), 3.77 (d, J=15.6 Hz, 3H), 3.43-3.11 (m, 6H), 3.05-2.85 (m, 1H), 2.59-2.23 (m, 2H).

Step 9: To a solution of compound E-13 (270 mg, 533 μmol, 95.4% purity, 1.00 eq) in H₂O (2.00 mL) was added HCl/dioxane (4.00 M, 9.00 mL, 67.6 eq), then the mixture was stirred at 60° C. for 2 hrs. The mixture was concentrated under reduced pressure to give a residue. The residue was purified by prep-HPLC (column: Phenomenex C18 150*25 mm*10 um; mobile phase: [water (NH₄HCO₃)-ACN]; B %: 3%-33%, 5 min). Compound E-14 (170 mg, 361 μmol, 67.8% yield, 99.7% purity) was obtained as white solid. LC-MS (M+H)⁺ 470.2.

Step 10: The compound E-14 (170 mg, 99.7% purity) was further separated by SFC (column: DAICEL CHIRALPAK AD (250 mm*30 mm, 10 um); mobile phase: [Neu-MeOH]; B %: 35%-35%, 5.0; 75 min). Compound 281 (31.77 mg, 64.6 μmol, 17.9% yield, 95.5% purity) was obtained as white solid. ¹H NMR (400 MHz, DMSO-d₆) δ 7.53 (d, J=7.6 Hz, 1H), 7.50-7.41 (m, 1H), 7.12-6.99 (m, 4H), 6.78 (br d, J=5.2 Hz, 1H), 6.40-6.23 (m, 1H), 5.64 (s, 1H), 4.59 (dd, J₁=8.8 Hz, J₂=4.4 Hz, 1H), 3.30-3.01 (m, 5H), 2.98-2.83 (m, 2H), 2.37-2.19 (m, 2H). LC-MS (M+H)⁺ 470.1. Compound 282 (30.30 mg, 63.3 μmol, 17.5% yield, 98.1% purity) was obtained as white solid. ¹H NMR (400 MHz, DMSO-d₆) δ 7.57 (d, J=7.6 Hz, 1H), 7.51-7.41 (m, 1H), 7.13-6.97 (m, 4H), 6.79 (d, J=6.8 Hz, 1H), 6.41-6.27 (m, 1H), 5.64 (s, 1H), 4.55 (dd, J₁=9.6 Hz, J_{2=4.4} Hz, 1H), 3.26-3.06 (m, 5H), 2.95-2.83 (m, 2H), 2.37-2.28 (m, 2H). LC-MS (M+H)⁺: 470.2.

The following compounds, set forth in Table 10, were also prepared using the synthetic procedures of Scheme E, or analogous procedures thereto:

TABLE 10

| Compound | |
|---|---|
| No. | Characterization Data |
| 285 | 484.3 [M + 1] |
| 286 | 484.1 [M + 1] |

### Example 18: Synthesis of Compound 169

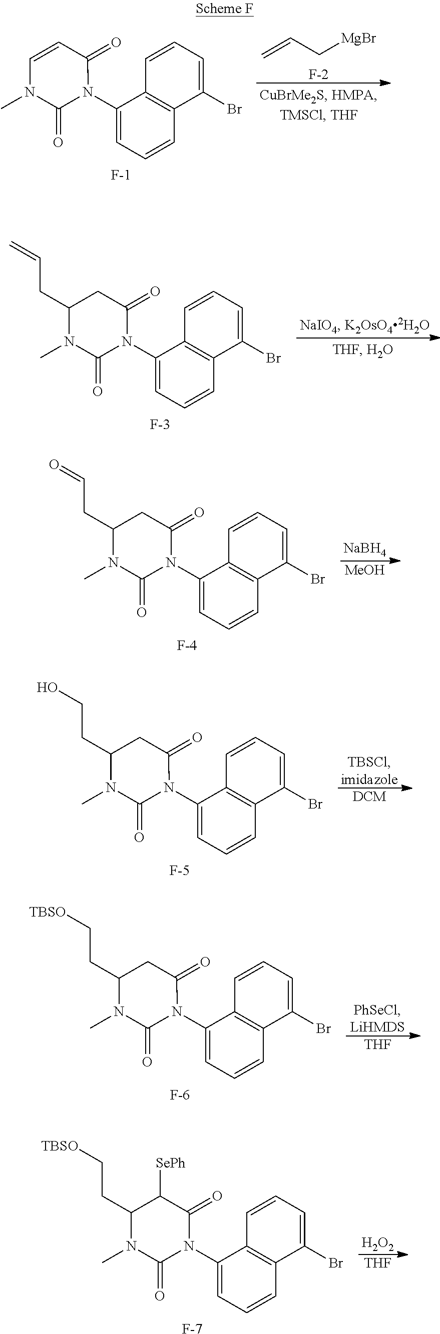

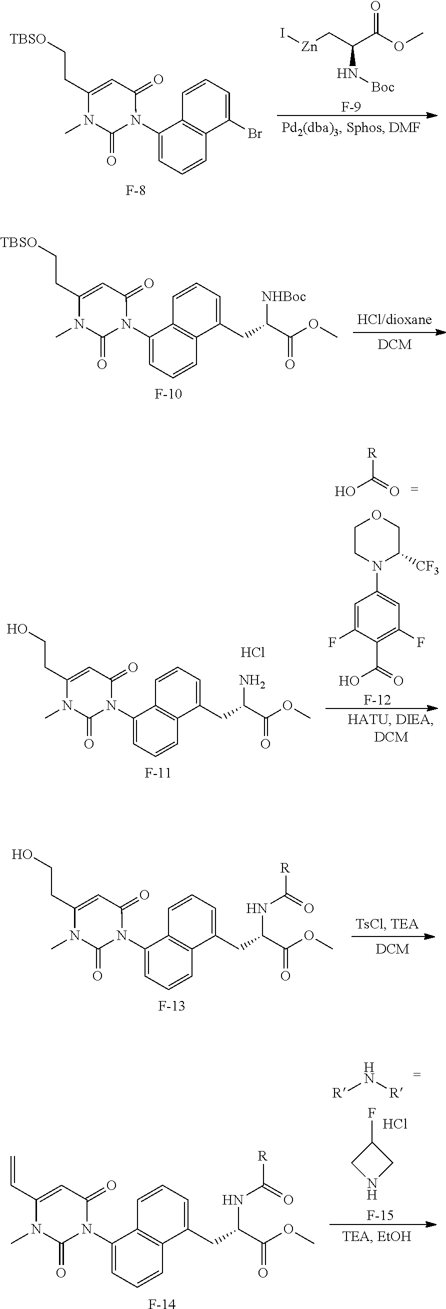

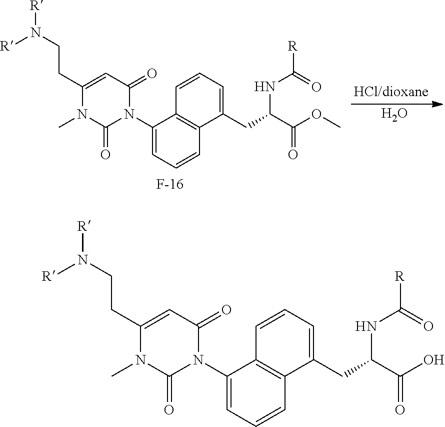

Step 1: To a solution of CuBr·Me₂S (1.86 g, 9.06 mmol, 0.200 eq) in THF (240 mL) was added compound F-2 (1.00M, 272 mL, 6.00 eq) at −78° C. The mixture was stirred at −78° C. for 1 hr, then compound 1 (15.0 g, 45.3 mmol, 1.00 eq), TMSCl (19.7 g, 181 mmol, 23.0 mL, 4.00 eq), HMPA (40.6 g, 227 mmol, 39.8 mL, 5.00 eq) in THF (240 mL) was added dropwise to the mixture at −78° C., and the mixture was stirred at 0° C. for 2 hrs under N₂. The reaction mixture was poured into ice water H₂O (500 mL), then extracted with ethyl acetate (500 mL*3), the combined organic phase was washed with brine (800 mL*2), dried over Na₂SO₄ and concentrated to give a residue. The residue was purified by Prep-HPLC (column: YMC Triart C18 70*250 mm*7 um; mobile phase: [water (FA)-ACN]; B %: 35%-65%, 20 min). Compound F-3 (6.50 g, 17.4 mmol, 38.4% yield) was obtained as yellow solid. LC-MS (M+H)⁻ 374.9. ¹H NMR (400 MHz, DMSO-d₆) δ 8.21 (d, J=8.4 Hz, 1H), 7.93 (t, J=7.6 Hz, 1H), 7.76-7.73 (m, 2H), 7.52-7.42 (m, 2H), 5.95-5.86 (m, 1H), 5.30-5.22 (m, 2H), 3.85-3.81 (m, 1H), 3.51 (dd, 1=16.4 Hz, J₂=6.8 Hz, 1H), 3.31-3.29 (m, 1H), 3.03 (d, J=4.0 Hz, 3H), 2.76-2.66 (m, 2H).

Step 2: To a solution of compound F-3 (5.50 g, 14.7 mmol, 1.00 eq) in THF (55 mL) and H₂O (55 mL) was added K₂OsO₄·2H₂O (543 mg, 1.47 mmol, 0.100 eq) and NaIO₄ (9.46 g, 44.2 mmol, 2.45 mL, 3.00 eq) at 0° C., then the mixture was stirred at 25° C. for 2 hrs. The mixture was diluted with brine (100 mL) and extracted with ethyl acetate (100 mL*3), the combined organic phase was washed with brine (200 mL*2), dried over Na₂SO₄ and concentrated to give a residue. Compound F-4 (5.50 g, crude) was obtained as yellow solid. LC-MS (M+H)⁺: 377.0. ¹H NMR (400 MHz, CDCl₃) δ 9.92 (d, J=20.0 Hz, 1H), 8.37 (d, J=8.0 Hz, 1H), 7.82 (d, J=6.4 Hz, 1H), 7.67-7.65 (m, 1H), 7.53-7.52 (m, 1H), 7.37-7.34 (m, 2H), 4.26-4.22 (m, 1H), 3.33-3.28 (m, 1H), 3.16-3.13 (m, 4H), 3.06-3.04 (m, 1H), 2.89 (dd, J₁=16.4 Hz, J₂=1.2 Hz, 1H).

Step 3: To a solution of compound F-4 (5.50 g, 14.7 mmol, 1.00 eq) in MeOH (55 mL) was added NaBH₄ (1.11 g, 29.3 mmol, 2.00 eq) at 0° C., then the mixture was stirred at 25° C. for 0.5 hr. The mixture was diluted with brine (100 mL) and extracted with dichloromethane (100 mL*3), the combined organic phase was washed with brine (100 mL*2), dried over Na₂SO₄ and concentrated to give a residue. Compound F-5 (5.50 g, crude) was obtained as yellow solid. LC-MS (M+H)⁺: 377.0.

Step 4: To a solution of compound F-6 (5.50 g, 14.6 mmol, 1.00 eq) in DCM (55 mL) was added TBSCl (3.30 g, 21.9 mmol, 2.68 mL, 1.50 eq) and imidazole (1.99 g, 29.2 mmol, 2.00 eq), then the mixture was stirred at 25° C. for 2 hrs. The mixture was diluted with H₂O (100 mL) and extracted with dichloromethane (100 mL*3), the combined organic phase was washed with brine (150 mL*2), dried over Na₂SO₄ and concentrated to give a residue. The residue was purified by column chromatography (SiO₂, Petroleum ether:Ethyl acetate=100:1 to 1:1, R_{f}=0.45, 0.50). Compound F-6 (5.00 g, 10.2 mmol, 69.8% yield) was obtained as yellow solid. LC-MS (M−H)⁺ 493.2. ¹H NMR (400 MHz, CDCl₃) δ 8.35 (d, J=8.8 Hz, 1H), 7.80 (d, J=7.6 Hz, 1H), 7.65-7.63 (m, 1H), 7.57-7.55 (m, 1H), 7.35-7.31 (m, 2H), 3.86-3.80 (m, 3H), 3.25-3.23 (m, 1H), 3.17 (d, J=4.0 Hz, 3H), 2.97-2.91 (m, 1H), 2.10-2.09 (m, 1H), 1.95-1.91 (m, 1H), 0.94 (d, J=2.4 Hz, 9H), 0.11 (dd, J₁=4.2 Hz, J₂=2.4 Hz, 6H).

Step 5: To a solution of compound F-6 (5.00 g, 10.2 mmol, 1.00 eq) in THF (50 mL) was added LiHMDS (1.00 M, 40.7 mL, 4.00 eq) at −78° C., the mixture was stirred at −78° C. for 0.5 hr, then PhSeCl (3.90 g, 20.4 mmol, 2.00 eq) in THF (20 mL) was added dropwise to the mixture at −78° C., the mixture was stirred at 0° C. for 1 hr. A solution of saturated NH₄Cl (80 mL) was added to the mixture, then extracted with ethyl acetate (80 mL*3), the combined organic phase was washed with brine (100 mL*2), dried over Na₂SO₄ and concentrated to give a residue. The residue was purified by column chromatography (SiO₂, Petroleum ether:Ethyl acetate=100:1 to 3:1, R_{f}=0.40). Compound F-7 (6.00 g, 9.28 mmol, 91.2% yield) was obtained as yellow solid. LC-MS (M−H)⁺ 647.1. ¹H NMR (400 MHz, DMSO-d₆) δ 8.23 (d, J=8.4 Hz, 1H), 7.95-7.68 (m, 5H), 7.51-7.23 (m, 5H), 4.51 (dd, J₁=7.2 Hz, J₂=1.6 Hz, 1H), 3.98-3.96 (m, 1H), 3.86-3.80 (m, 2H), 3.02 (d, J=16.0 Hz, 3H), 2.06-1.99 (m, 2H), 0.84 (s, 9H), 0.05 (s, 6H).

Step 6: To a solution of compound F-7 (6.00 g, 9.28 mmol, 1.00 eq) in THF (120 mL) was added dropwise H₂O₂ (5.26 g, 46.4 mmol, 4.46 mL, 30.0% purity, 5.00 eq) at 0° C., then the mixture was stirred at 25° C. for 0.5 hr. A solution of saturated Na₂SO₃ (80 mL) was added to the mixture, then diluted with brine (80 mL) and extracted with ethyl acetate (150 mL*3), the combined organic phase was washed with brine (100 mL*2), dried over Na₂SO₄ and concentrated to give a residue. The residue was purified by column chromatography (SiO₂, Petroleum ether:Ethyl acetate=100:1 to 1:1, R_{f}=0.45). Compound F-8 (3.00 g, 6.13 mmol, 66.0% yield) was obtained as white solid. LC-MS (M+H)⁺ 491.1. ¹H NMR (400 MHz, DMSO-d₆) δ 8.25 (d, J=8.4 Hz, 1H), 7.95 (d, J=7.2 Hz, 1H), 7.79 (dd, J₁=8.4 Hz, J₂=7.2 Hz, 1H), 7.54 (dd, J₁=20.8 Hz, J₂=8.4 Hz, 2H), 7.42 (dd, J₁=8.4 Hz, J₂=7.6 Hz, 1H), 5.82 (m, 1H), 3.96 (t, J=6.4 Hz, 2H), 3.41 (s, 3H), 2.91 (t, J=6.4 Hz, 2H), 0.90 (s, 9H), 0.10 (s, 6H).

Step 7: To a solution of compound F-8 (2.20 g, 4.49 mmol, 1.00 eq) in DMF (30 mL) was added SPhos (369 mg, 898 μmol, 0.200 eq) and Pd₂(dba)₃ (411 mg, 449 μmol, 0.100 eq) under N₂. Compound F-9 (5.31 g, 13.5 mmol, 3.00 eq) in DMF (30 mL) was added to the mixture. The mixture was stirred at 90° C. for 8 hrs. The mixture was diluted with H₂O (100 mL) and extracted with ethyl acetate (100 mL*3), the combined organic phase was washed with brine (100 mL*3), dried over Na₂SO₄ and concentrated to give a residue. The residue was purified by column chromatography (SiO₂, Petroleum ether:Ethyl acetate=100:1 to 1:1). Compound 10 (1.50 g, 2.45 mmol, 54.6% yield) was obtained as yellow solid. ¹H NMR (400 MHz, CDCl₃) δ 8.19 (d, J=8.8 Hz, 1H), 7.64 (t, J=8.0 Hz, 1H), 7.49 (d, J=8.4 Hz, 1H), 7.39 (d, J=7.2 Hz, 2H), 7.30 (d, J=7.2 Hz, 1H), 5.84 (m, 1H), 5.04 (d, J=8.0 Hz, 1H), 4.73-4.71 (m, 1H), 4.01 (t, J=6.0 Hz, 2H), 3.70-3.63 (m, 4H), 3.54-3.49 (m, 4H), 2.88-2.86 (m, 2H), 1.57-1.44 (m, 9H), 0.94 (s, 9H), 0.13 (s, 6H).

Step 8: To a solution of compound F-10 (300 mg, 490 μmol, 1.00 eq) in DCM (3.00 mL) was added HCl/dioxane (4.00 M, 3.00 mL, 24.5 eq) at 0° C., then the mixture was stirred at 25° C. for 1 hr. The mixture was concentrated to give a residue. Compound F-11 (210 mg, crude, HCl) was obtained as yellow solid. LC-MS (M+H)⁺ 398.1.

Step 9: To a solution of compound F-12 (158 mg, 507 μmol, 1.10 eq) in DCM (3.00 mL) was added DIEA (179 mg, 1.38 mmol, 240 μL, 3.00 eq) and HATU (3.50 mg, 922 μmol, 2.00 eq), the mixture was stirred at 25° C. for 0.5 hr, then compound F-11 (200 mg, 461 μmol, 1.00 eq, HCl) was added and the mixture was stirred at 25° C. for 1 hr. The mixture was diluted with H₂O (20 mL) and extracted with a mixture of dichloromethane and methanol (Dichloromethane:Methanol=10:1, 30 mL*3), the combined organic phase was dried over Na₂SO₄ and concentrated to give a residue. The residue was purified by Prep-TLC (Dichloromethane:Methanol=10:1, R_{f}=0.60). Compound F-13 (250 mg, 362 μmol, 78.5% yield) was obtained as yellow solid. LC-MS (M+H)⁺ 691.2. ¹H NMR (400 MHz, DMSO-d₆) δ 9.09 (d, J=7.6 Hz, 1H), 8.19 (d, J=8.4 Hz, 1H), 7.69 (t, J=7.6 Hz, 1H), 7.47-7.41 (m, 4H), 6.78 (d, J=11.6 Hz, 2H), 5.78 (d, J=2.8 Hz, 1H), 4.98 (t, J=5.6 Hz, 1H), 4.92-4.91 (m, 1H), 4.73-4.71 (m, 1H), 4.16 (d, J=13.2 Hz, 1H), 3.96 (dd, J₁=10.8 Hz, J_{2=3.6} Hz, 1H), 3.81-3.78 (m, 2H), 3.73-3.70 (m, 1H), 3.66 (s, 3H), 3.62-3.61 (m, 1H), 3.56-3.53 (m, 1H), 3.46-3.44 (m, 1H), 3.39 (m, 3H), 3.16-3.14 (m, 2H), 2.84 (t, J=6.0 Hz, 2H).

Step 10: To a solution of compound F-13 (200 mg, 290 μmol, 1.00 eq) in DCM (5 mL) was added TEA (87.9 mg, 869 μmol, 121 μL, 3.00 eq) and TsCl (82.8 mg, 434 μmol, 1.50 eq), then the mixture was stirred at 25° C. for 2 hrs. The mixture was diluted with H₂O (20 mL) and extracted with dichloromethane and methanol (Dichloromethane:Methanol=10:1, 20 mL*3), the combined organic phase was dried over Na₂SO₄ and concentrated to give a residue. The residue was purified by Prep-TLC (Dichloromethane:Methanol=10:1, R_{f}=0.60). Compound F-14 (100 mg, 149 μmol, 51.3% yield) was obtained as yellow solid. LC-MS (M+H)⁻ 673.2. ¹H NMR (400 MHz, DMSO-d₆) δ 9.09 (d, J=8.0 Hz, 1H), 8.20 (d, J=8.4 Hz, 1H), 7.69 (t, J=7.6 Hz, 1H), 7.51-7.41 (m, 4H), 6.89 (dd, J₁=17.2 Hz, J₂=10.8 Hz, 1H), 6.78 (d, J=12.0 Hz, 2H), 6.13-5.74 (m, 2H), 4.93-4.90 (m, 1H), 4.73-4.72 (m, 1H), 4.16 (d, J=12.8 Hz, 1H), 3.96 (dd, J₁-10.8 Hz, J₂-3.6 Hz, 1H), 3.73-3.66 (m, 5H), 3.58-3.52 (m, 1H), 3.47-3.37 (m, 5H), 3.18-3.16 (m, 2H).

Step 11: To a solution of compound F-14 (80.0 mg, 119 μmol, 1.00 eq) in EtOH (3.00 mL) was added TEA (60.2 mg, 595 μmol, 82.8 uL, 5.00 eq) and compound F-15 (39.8 mg, 357 μmol, 3.00 eq, HCl), then the mixture was stirred at 80° C. for 8 hrs. The mixture was concentrated to give a residue. Then diluted with H₂O (20 mL) and extracted with ethyl acetate (20 mL*3), the combined organic phase was washed with brine (20 mL*2), dried over Na₂SO₄ and concentrated to give a residue. Compound F-16 (85.0 mg, crude) was obtained as yellow solid. LC-MS (M+H)⁺ 748.5.

Step 12: To a solution of compound F-16 (85.0 mg, 114 μmol, 1.00 eq) in MeCN (0.500 mL) was added HCl (4.00 M, 1.00 mL, 35.2 eq), then the mixture was stirred at 60° C. for 3 hrs. The mixture was concentrated to give a residue. The residue was purified by Prep-HPLC (column: Welch Ultimate AQ-C18 150*30 mm*5 um; mobile phase: [water (HCl)-ACN]; B %: 14%-44%, 11 min) and Prep-HPLC (column: Waters xbridge 150*25 mm 10 um; mobile phase: [water (NH₄HCO₃)-ACN]; B %: 10%-40%, 9 min). Compound 169 (29.16 mg, 37.7 μmol, 33.1% yield, 94.8% purity) was obtained as white solid. ¹H NMR (400 MHz, DMSO-d₆) δ 8.83 (br s, 1H), 8.25 (d, J=8.8 Hz, 1H), 7.67 (t, J=8.0 Hz, 1H), 7.48-7.40 (m, 4H), 6.76 (d, J=11.6 Hz, 2H), 5.79 (d, J=4.4 Hz, 1H), 5.28-5.12 (m, 1H), 4.92-4.89 (m, 1H), 4.63-4.62 (m, 1H), 4.16 (d, J=12.8 Hz, 1H), 3.95 (dd, J₁=10.8 Hz, J₂=3.6 Hz, 1H), 3.72-3.64 (m, 5H), 3.41-3.37 (m, 5H), 3.32-3.30 (m, 2H), 3.17-3.13 (m, 1H), 2.81 (t, J=6.8 Hz, 2H), 2.69 (t, J=6.8 Hz, 2H). LC-MS (M+H)+734.3.

The following compounds, set forth in Table 11, were also prepared using the synthetic procedures of Scheme F, or analogous procedures thereto:

TABLE 11

| Comp. | Structure and |
|---|---|
| No. | Characterization Data |
| 116 | 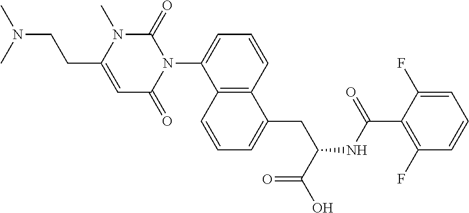 [SMILES: CN(C)CCc1cc(=O)n(-c2cccc3c(C[C@H](NC(=O)c4c(F)cccc4F)C(=O)O)cccc23)c(=O)n1C] |
| | 551.3 [M + 1] |
| 1056 | 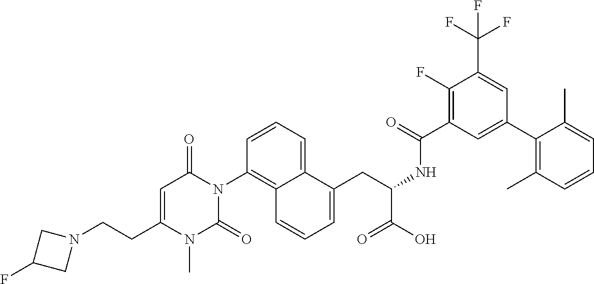 [SMILES: Cc1cccc(C)c1-c1cc(C(=O)N[C@@H](Cc2cccc3c(-n4c(=O)cc(CCN5CC(F)C5)n(C)c4=O)cccc23)C(=O)O)c(F)c(C(F)(F)F)c1] |
| | m/z = 735.2 |
| 1057 | 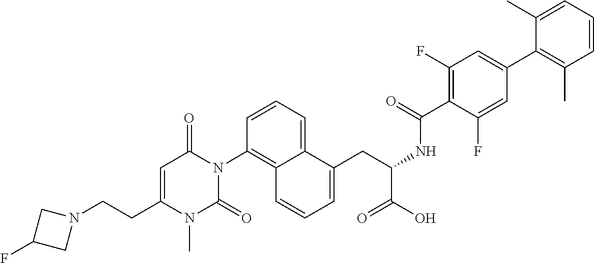 [SMILES: Cc1cccc(C)c1-c1cc(F)c(C(=O)N[C@@H](Cc2cccc3c(-n4c(=O)cc(CCN5CC(F)C5)n(C)c4=O)cccc23)C(=O)O)c(F)c1] |
| | m/ z = 685.3 |

### Example 19: Synthesis of Compound 117

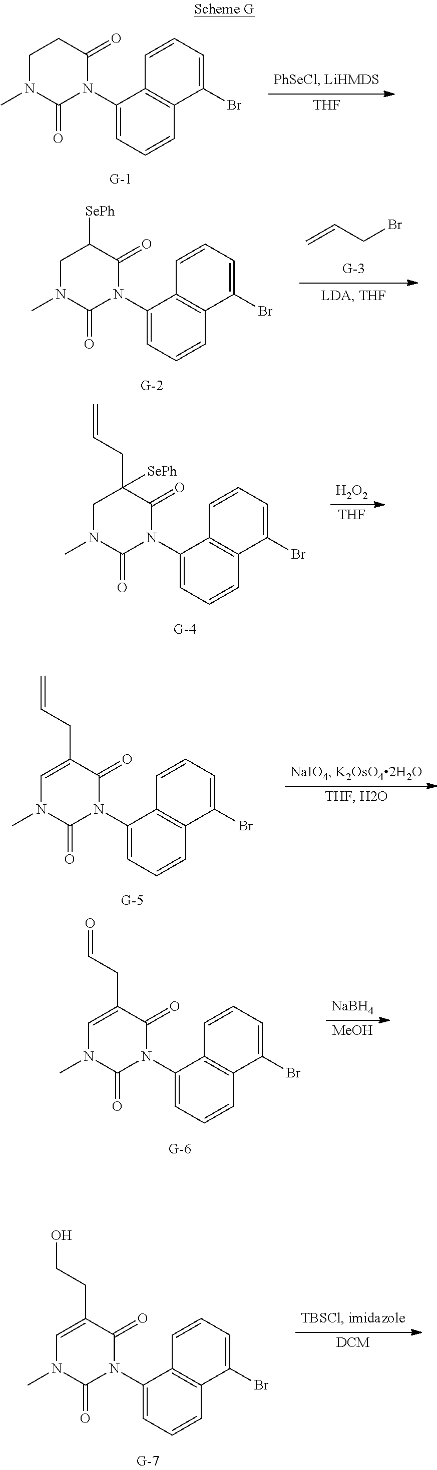

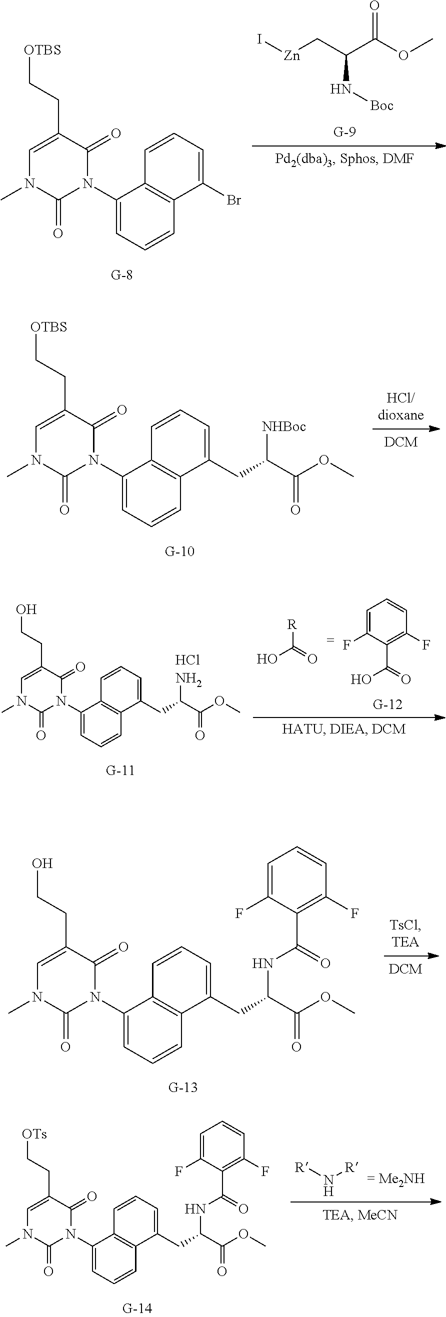

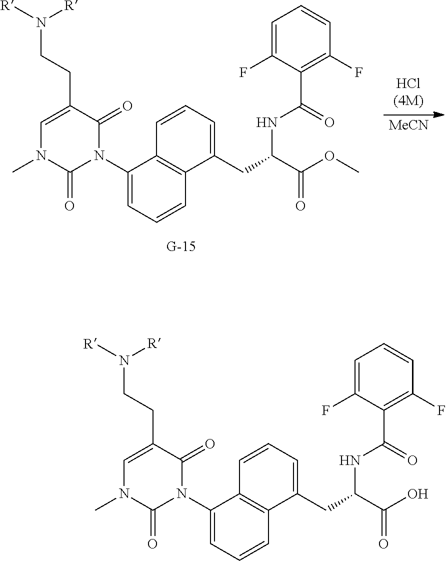

Step 1: To a solution of compound G-1 (29.0 g, 87.0 mmol, 1.00 eq) in THF (290 mL) was added LiHMDS (1.00 M, 174 mL, 2.00 eq) at −78° C., the mixture was stirred at −78° C. for 1 hr. Then PhSeCl (17.5 g, 91.4 mmol, 1.05 eq) in THF (175 mL) was added at −78° C. The mixture was stirred at 0° C. for 2 hrs. The mixture was added dropwise to saturated ice NH₄Cl (200 mL), then extracted with ethyl acetate (200 mL*3), the combined organic phase was washed with brine (300 mL*2), dried over Na₂SO₄ and concentrated to give a residue. The crude product was triturated with a mixture of petroleum ether and ethyl acetate (Petroleum ether:Ethyl acetate=10:1, 200 mL) at 25° C. for 30 min. Compound G-2 (34.0 g, crude) was obtained as yellow solid. LC-MS (M+H)⁺ 489.0. ¹H NMR (400 MHz, DMSO-d₆) δ 8.22-8.20 (m, 1H), 7.94-7.92 (m, 2H), 7.73-7.69 (m, 3H), 7.56-7.41 (m, 3H), 7.35-7.18 (m, 2H), 4.63-4.52 (m, 1H), 4.36-4.27 (m, 1H), 3.67-3.63 (m, 1H), 2.97 (d, J=15.2 Hz, 3H).

Step 2: To a solution of compound G-2 (18.0 g, 36.9 mmol, 1.00 eq) in THF (180 mL) was added LDA (2.00 M, 36.8 mL, 2.00 eq) at −78° C., the mixture was stirred at −78° C. for 0.5 hr. Then compound G-3 (5.35 g, 44.2 mmol, 1.20 eq) in THF (20 mL) was added at −78° C. The resulting mixture was stirred at 0° C. for 2 hrs. The mixture was added dropwise to a solution of saturated ice NH₄Cl (200 mL), then extracted with ethyl acetate (200 mL*3), the combined organic phase was washed with brine (200 mL*2), dried over Na₂SO₄ and concentrated to give a residue. The residue was purified by column chromatography (SiO₂, Petroleum ether:Ethyl acetate=100:1 to 1:1). TLC (Petroleum ether:Ethyl acetate=2:1, R_{f}=0.55, 0.60). Compound G-4 (12.0 g, crude) was obtained as yellow solid. LC-MS (M+H)⁻ 529.1.

Step 3: To a solution of compound G-4 (12.0 g, 22.7 mmol, 1.00 eq) in THF (240 mL) was added H₂O₂ (7.73 g, 68.1 mmol, 6.55 mL, 30.0% purity, 3.00 eq) at 0° C., then the mixture was stirred at 25° C. for 1 hr. A solution of saturated NaHCO₃ (100 mL) and Na₂SO₃ (100 mL) was added to the mixture, then extracted with ethyl acetate (200 mL*3), the combined organic phase was washed with brine (200 mL*2), dried over Na₂SO₄ and concentrated to give a residue. The residue was purified by column chromatography (SiO₂, Petroleum ether:Ethyl acetate=100:1 to 1:1). TLC (Petroleum ether:Ethyl acetate=1:1, R_{f}=0.40). Compound G-5 (5.50 g, 14.8 mmol, 65.2% yield) was obtained as yellow solid. LC-MS (M+H)⁻ 372.9. ¹H NMR (400 MHz, CDCl₃) δ 8.39 (d, J=8.4 Hz, 1H), 7.81 (dd, J₁=7.2 Hz, J₂=0.8 Hz, 1H), 7.70-7.68 (m, 1H), 7.53-7.50 (m, 1H), 7.45 (dd, J_{1=7.2} Hz, J₂=0.8 Hz, 1H), 7.34-7.32 (m, 1H), 7.14 (t, J=0.8 Hz, 1H), 5.98-5.90 (m, 1H), 5.25-5.19 (m, 2H), 3.47 (s, 3H), 3.19-3.17 (m, 2H).

Step 4: To a solution of compound G-5 (5.50 g, 14.8 mmol, 1.00 eq) in THF (55 mL) and H₂O (55 mL) was added K₂OsO₄·2H₂O (546 mg, 1.48 mmol, 0.100 eq) and NaIO₄ (9.51 g, 44.5 mmol, 2.46 mL, 3.00 eq) at 0° C., then the mixture was stirred at 25° C. for 1 hr. The mixture was diluted with brine (50 mL) and extracted with ethyl acetate (100 mL*3), the combined organic phase was washed with brine (200 mL*2), dried over Na₂SO₄ and concentrated to give a residue. Compound G-6 (5.50 g, crude) was obtained as yellow solid. ¹H NMR (400 MHz, CDCl₃) δ 9.80 (s, 1H), 8.40 (d, J=8.8 Hz, 1H), 7.82 (d, J=7.2 Hz, 1H), 7.70 (t, J=7.6 Hz, 1H), 7.52 (d, J=8.4 Hz, 1H), 7.46 (d, J=7.6 Hz, 1H), 7.37 (s, 1H), 7.34 (t, J=8.0 Hz, 1H), 3.55 (s, 2H), 3.48 (s, 3H).

Step 5: To a solution of compound G-6 (5.50 g, 14.7 mmol, 1.00 eq) in MeOH (55 mL) was added NaBH₄ (1.12 g, 29.5 mmol, 2.00 eq) at 0° C., then the mixture was stirred at 25° C. for 1 hr. A solution of saturated NH₄Cl (50 mL) was added to the mixture, then the mixture was concentrated under reduced pressure to remove MeOH, then extracted with ethyl acetate (100 mL*3), the combined organic phase was washed with brine (100 mL*2), dried over Na₂SO₄ and concentrated to give a residue. Compound G-7 (5.50 g, crude) was obtained as yellow solid. LC-MS (M+H)⁻ 376.8.

Step 6: To a solution of compound G-7 (5.50 g, 14.7 mmol, 1.00 eq) in DCM (55 mL) was added TBSCl (2.87 g, 19.1 mmol, 2.34 mL, 1.30 eq) and imidazole (2.00 g, 29.3 mmol, 2.00 eq), then the mixture was stirred at 25° C. for 1 hr. The mixture was diluted with H₂O (100 mL) and extracted with DCM (100 mL*3). The combined organic phase was washed with brine (150 mL*2), dried over Na₂SO₄ and concentrated to give a residue. The residue was purified by column chromatography (SiO₂, Petroleum ether:Ethyl acetate=100:1 to 3:1). TLC (Petroleum ether:Ethyl acetate=3:1, R_{f}=0.50). Compound G-8 (3.20 g, 6.54 mmol, 44.6% yield) was obtained as yellow solid. LC-MS (M+H)⁺ 491.0. ¹H NMR (400 MHz, CDCl₃) δ 8.39 (d, J=8.8 Hz, 1H), 7.81 (dd, J₁=7.6 Hz, J₂=0.8 Hz, 1H), 7.70-7.67 (m, 1H), 7.51 (d, J=8.4 Hz, 1H), 7.44 (dd, J₁=7.6 Hz, J₂=0.8 Hz, 1H), 7.33-7.32 (m, 2H), 3.80 (d, J=6.0 Hz, 2H), 3.46 (s, 3H), 2.61 (t, J=5.6 Hz, 2H), 0.93 (s, 9H), 0.07 (d, J=2.0 Hz, 6H).

Step 7: To a solution of compound G-8 (3.00 g, 6.13 mmol, 1.00 eq) in DMF (30 mL) was added Sphos (503 mg, 1.23 mmol, 0.200 eq) and Pd₂(dba)₃ (561 mg, 613 μmol, 0.100 eq) under N₂, then compound G-9 (7.25 g, 18.4 mmol, 3.00 eq) in DMF (30 mL) was added and the mixture was stirred at 80° C. for 1 hr. The mixture was filtered and the filtrate was concentrated to give a residue. The residue was purified by column chromatography (SiO₂, Petroleum ether:Ethyl acetate=100:1 to 1:1). TLC (Petroleum ether:Ethyl acetate=1:1, R_{f}=0.50). Compound G-10 (3.00 g, 4.90 mmol, 80.0% yield) was obtained as yellow solid. LC-MS (M+H-56)⁺ 556.4. ¹H NMR (400 MHz, CDCl₃) δ 8.20-8.14 (m, 2H), 7.66-7.64 (m, 1H), 7.47-7.29 (m, 5H), 4.73-4.65 (m, 1H), 3.81-3.75 (m, 3H), 3.66-3.64 (m, 1H), 3.07 (s, 3H), 2.97 (d, J=0.8 Hz, 3H), 2.62-2.58 (m, 2H), 1.42 (s, 6H), 1.10 (br s, 3H), 0.93 (s, 9H), 0.07 (d, J=2.4 Hz, 6H).

Step 8: To a solution of compound G-10 (1.00 g, 1.63 mmol, 1.00 eq) in DCM (5 mL) was added HCl/dioxane (4.00 M, 5.00 mL, 12.2 eq) at 0° C., then the mixture was stirred at 25° C. for 1 hr. The mixture was concentrated to give a residue. Compound G-11 (700 mg, crude, HCl) was obtained as yellow solid. LC-MS (M+H)⁻ 398.2.

Step 9: To a solution of compound G-12 (255 mg, 1.61 mmol, 1.00 eq) in DCM (7.00 mL) was added HATU (1.23 g, 3.23 mmol, 2.00 eq) and DIEA (626 mg, 4.84 mmol, 843 μL, 3.00 eq), the mixture was stirred at 25° C. for 0.5 hr, then compound G-11 (700 mg, 1.61 mmol, 1.00 eq, HCl) was added and the mixture was stirred at 25° C. for 0.5 hr. The mixture was diluted with saturated NaHCO₃ (30 mL) and H₂O (20 mL), then extracted with a mixture of DCM and MeOH (DCM:MeOH=10:1, 50 mL*3), the combined organic phase was dried over Na₂SO₄ and concentrated to give a residue. The residue was purified by Prep-TLC (Petroleum ether:Ethyl acetate=0:1, R_{f}=0.20). Compound G-13 (600 mg, crude) was obtained as yellow oil. LC-MS (M+H)⁺ 538.3. ¹H NMR (400 MHz, CDCl₃) δ 8.31 (d, J=8.4 Hz, 1H), 8.02 (s, 1H), 7.65 (t, J=8.0 Hz, 1H), 7.48-7.46 (m, 1H), 7.43-7.35 (m, 4H), 6.95 (t, J=8.4 Hz, 2H), 6.68 (d, J=6.8 Hz, 1H), 5.24-5.18 (m, 1H), 3.86-3.82 (m, 2H), 3.76-3.74 (m, 2H), 3.66 (s, 3H), 3.48 (d, J=2.8 Hz, 3H), 2.68-2.65 (m, 2H).

Step 10: To a solution of compound G-13 (600 mg, 1.12 mmol, 1.00 eq) in DCM (6.00 mL) was added TsCl (319 mg, 1.67 mmol, 1.50 eq) and TEA (226 mg, 2.23 mmol, 311 μL, 2.00 eq), then the mixture was stirred at 25° C. for 1 hr. The mixture was diluted with H₂O (30 mL) and extracted with a mixture of DCM and MeOH (DCM:MeOH=10:1, 30 mL*3), the combined organic phase was dried over Na₂SO₄ and concentrated to give a residue. The residue was purified by Prep-TLC Petroleum ether:Ethyl acetate=1:5, R_{f}=0.50). Compound G-14 (250 mg, 361 μmol, 32.4% yield) was obtained as yellow solid. LC-MS (M+H)⁺692.5. ¹H NMR (400 MHz, CDCl₃) δ 8.31 (dd, J_{1=8.8} Hz, J₂=3.2 Hz, 1H), 7.79-7.76 (m, 3H), 7.48-7.46 (m, 1H), 3.40-7.30 (m, 7H), 6.95 (t, J=8.0 Hz, 2H), 6.63 (t, J=7.2 Hz, 1H), 5.25-5.20 (m, 1H), 4.27-4.22 (m, 2H), 3.79-3.74 (m, 2H), 3.63 (s, 3H), 3.48 (d, J=2.8 Hz, 3H), 2.77 (t, J=5.6 Hz, 2H), 2.45 (s, 3H).

Step 11: To a solution of compound G-14 (200 mg, 289 μmol, 1.00 eq) in MeCN (2.00 mL) was added Me₂NH (2.00 M, 5.00 mL, 34.6 eq), then the mixture was stirred at 25° C. for 12 hrs. The mixture was concentrated to give a residue. The residue was purified by Prep-HPLC (column: Welch Xtimate C18 150*25 mm*5 um; mobile phase. [water (FA)-ACN]; B %: 8%-38%, 10 min). Compound G-15 (80.0 mg, 131 μmol, 45.3% yield, FA) was obtained as yellow solid. LC-MS (M+H)⁻ 565.4. ¹H NMR (400 MHz, DMSO-d₆+D₂O) δ 8.19 (d, J=7.6 Hz, 1H), 7.81 (s, 1H), 7.70 (t, J=7.2 Hz, 1H), 7.50-7.41 (m, 4H), 7.15-7.09 (m, 3H), 4.79-4.76 (m, 1H), 3.70-3.66 (m, 4H), 3.42-3.38 (m, 1H), 3.34 (s, 3H), 2.98-2.96 (m, 2H), 2.60-2.59 (m, 8H).

Step 12: To a solution of compound G-15 (80.0 mg, 131 μmol, 1.00 eq, FA) in MeCN (1.00 mL) was added HCl (4.00 M, 1.00 mL, 30.5 eq), then the mixture was stirred at 60° C. for 2 hrs. The mixture was concentrated to give a residue. The residue was purified by Prep-HPLC (column: Phenomenex luna C18 150*25 mm*10 um; mobile phase: [water (FA)-ACN]; B %: 1%-31%, 9 min). Compound 117 (37.06 mg, 63.5 μmol, 48.5% yield, 94.4% purity) was obtained as yellow solid. LC-MS (M+H)⁺ 551.4. ¹H NMR (400 MHz, DMSO-d₆) δ 9.11 (d, J=7.6 Hz, 1H), 8.27 (d, J=8.8 Hz, 1H), 7.84 (s, 1H), 7.68 (t, J=7.6 Hz, 1H), 7.48-7.40 (m, 5H), 7.12 (t, J=8.0 Hz, 2H), 4.71-4.66 (m, 1H), 3.74 (dd, J₁=10.4 Hz, J₂=4.0 Hz, 1H), 3.39-3.34 (m, 4H), 2.61-2.59 (m, 2H), 2.32-2.29 (m, 8H).

The following compounds, set forth in Table 12, were also prepared using the synthetic procedures of Scheme G, or analogous procedures thereto:

TABLE 12

| Compound | |
|---|---|
| No. | Characterization Data |
| 118 | 581.1 [M + 1] |
| 136 | 520.3 [M + 1] |
| 137 | 524.2 [M + 1] |
| 141 | 520.2 [M + 1] |

### Example 20: Synthesis of Compound 119

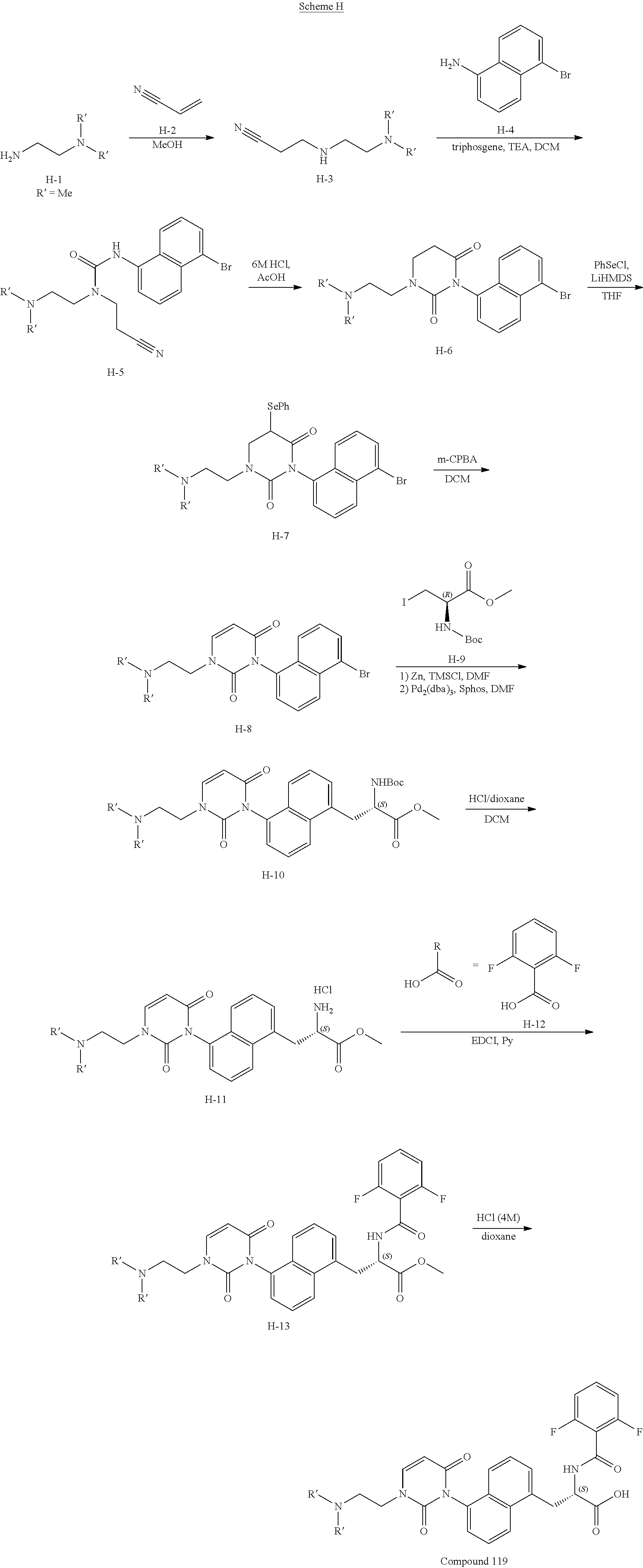

Step 1: To a solution of compound H-1 (8.00 g, 91.0 mmol, 9.91 mL, 1.00 eq) in MeOH (40 mL) was added compound H-2 (5.78 g, 109 mmol, 7.22 mL, 1.20 eq) at 0-5° C. The mixture was stirred at 0-5° C. for 6 hrs. The reaction mixture was concentrated under reduced pressure to remove MeOH to give a residue. Compound H-3 (12.3 g, 86.8 mmol, 95.7% yield) was obtained as colorless oil. ¹H NMR (400 MHz, CDCl₃) δ 2.95-2.87 (m, 2H), 2.72-2.65 (m, 2H), 2.53-2.46 (m, 2H), 2.42-2.35 (m, 2H), 2.23-2.16 (m, 6H).

Step 2: To a solution of compound H-4 (4.00 g, 18.0 mmol, 1.00 eq) in DCM (160 mL) was added TEA (5.47 g, 54.0 mmol, 7.52 mL, 3.00 eq) and a solution of bis(trichloromethyl) carbonate (1.88 g, 6.32 mmol, 0.351 eq) in DCM (40 mL) at 0-5° C., the mixture was stirred at 0-5° C. for 2 hrs. Then the mixture was added drop-wise a solution of compound H-3 (5.00 g, 35.4 mmol, 1.97 eq) at 0-5° C. The mixture was stirred at 20-25° C. for 2 hrs. The reaction mixture was quenched by addition saturated NaHCO₃ solution 300 mL at 0-5° C., and then extracted with DCM (400 mL*3). The combined organic layers were dried over Na₂SO₄, filtered and concentrated under reduced pressure to give a residue. The residue was purified by prep-HPLC (FA condition; column: YMC Triart C18 250*50 mm*7 um; mobile phase: [water (0.225% FA)-ACN]; B %: 3%-40%, 25 min). Compound H-5 (5.36 g, 13.8 mmol, 76.4% yield) was obtained as a brown solid. LC-MS (M+H)⁺ 391.0. ¹H NMR (400 MHz, CDCl₃) δ 8.13 (s, 1H), 7.94 (d, J=8.4 Hz, 1H), 7.79 (d, J=7.2 Hz, 1H), 7.54-7.62 (m, 2H), 7.36-7.30 (m, 1H), 3.84 (t, J=5.6 Hz, 2H), 3.72 (t, J=6.4 Hz, 2H), 3.06 (t, J=5.6 Hz, 2H), 2.81 (t, J=6.0 Hz, 2H), 2.60 (s, 6H).

Step 3: To a solution of compound H-5 (5.36 g, 13.8 mmol, 1.00 eq) in HCl (100 mL, 6M) was added AcOH (10.5 g, 174 mmol, 10.0 mL, 12.7 eq). The mixture was stirred at 100-105° C. for 36 hrs. The reaction mixture was concentrated under reduced pressure to give a residue. The residue was purified by prep-HPLC (FA condition; column: YMC Triart C18 250*50 mm*7 um; mobile phase: [water (0.225% FA)-ACN]; B %: 12%-35%, 15 min). Compound H-6 (3.70 g, 9.48 mmol, 68.8% yield, - purity) was obtained as a yellow solid. LC-MS (M+H)⁺: 392.1. ¹H NMR (400 MHz, DMSO-d₆) δ 8.20 (d, J=8.6 Hz, 1H), 7.92 (dd, J₁=7.2 Hz, J₂=0.8 Hz, 1H), 7.75 (dd, J₁=8.8 Hz, J₂=8.0 Hz, 2H), 7.51 (dd, J₁=7.2 Hz, J₂=0.8 Hz, 1H), 7.43 (dd, J₁=8.4 Hz, J₂=7.6 Hz, 1H), 3.66-3.78 (m, 2H), 3.50-3.61 (m, 2H), 3.08-2.95 (m, 1H), 2.89-2.96 (m, 1H), 2.61-2.69 (m, 2H), 2.33 (s, 6H).

Step 4: To a solution of compound H-6 (1.00 g, 2.56 mmol, 1.00 eq) in THF (20 mL) was added dropwise LiHMDS (1.00 M, 10.3 mL, 4.02 eq) at −78 to −70° C. and stirred at −78 to −70° C. for 1 hr under N₂ atmosphere, and then PhSeCl (540 mg, 2.82 mmol, 1.10 eq) in THF (5 mL) was added at −78 to −70° C. The resulting mixture was stirred at −78 to −70° C. for 2 hrs. The reaction mixture was quenched by addition saturated NH₄Cl solution 50 mL at 0-5° C., and then extracted with EtOAc (100 mL*3). The combined organic layers were dried over Na₂SO₄, filtered and concentrated under reduced pressure to give a residue. The residue was purified by prep-HPLC (FA condition; column: YMC Triart C18 250*50 mm*7 um; mobile phase: [water (0.225% FA)-ACN]; B %: 22%-52%, 20 min). Compound H-7 (1.00 g, 1.83 mmol, 71.6% yield, - purity) was obtained as a brown solid. LC-MS (M+H)⁻ 545.9. ¹H NMR (400 MHz, CDCl₃) δ 8.35 (d, J=8.4 Hz, 1H), 8.03 (d, J=8.4 Hz, 1H), 7.62-7.85 (m, 4H), 7.46-7.60 (m, 1H), 7.28-7.45 (m, 4H), 4.39-4.54 (m, 1H), 4.15-4.34 (m, 1H), 3.92-4.12 (m, 1H), 3.86-3.72 (m, 1H), 3.59-3.72 (m, 1H), 2.89-3.05 (m, 2H), 2.60-2.69 (m, 6H).

Step 5: To a solution of compound H-7 (550 mg, 1.01 mmol, 1.00 eq) in DCM (15 mL) was added m-CPBA (307 mg, 1.51 mmol, 85.0% purity, 1.50 eq) in DCM (5 mL) at −20-−25° C. under N₂ atmosphere. The mixture was stirred at −20-−25° C. for 1.5 hrs. The reaction mixture was quenched by saturated NaHCO₃ solution 50 mL at 0-5° C., and then extracted with DCM (60 mL*3). The combined organic layers were washed with saturated Na₂SO₃ solution (50 mL*3), dried over Na₂SO₄, filtered and concentrated under reduced pressure to give a residue. Compound H-8 (390 mg, crude) was obtained as a brown solid. LC-MS (M+H)⁺ 390.0. ¹H NMR (400 MHz, CDCl₃) δ 8.39 (d, J=8.8 Hz, 1H), 7.81 (d, J=7.2 Hz, 1H), 7.70 (dd, 1=8.4 Hz, J₂=7.6 Hz, 1H), 7.53 (d, J=8.4 Hz, 1H), 7.41-7.50 (m, 2H), 7.30-7.36 (m, 1H), 5.92 (d, J=8.0 Hz, 1H), 3.87-3.95 (m, 2H), 2.65 (t, J=6.0 Hz, 2H), 2.32 (s, 6H).

Step 6: To zinc (1.12 g, 17.1 mmol, 1.87 eq) was added TMSCl (300 mg, 2.76 mmol, 350 μL, 0.303 eq) in DMF (15 mL) and stirred at 20-25° C. for 30 min. And the supernatant liquor was removed by syringe. Then a mixture of compound H-9 (3.00 g, 9.11 mmol, 1.00 eq) in DMF (15 mL) was added to the precipitate. And the resulting mixture was stirred at 20-25° C. for 2 hrs. Compound [(2R)-2-(tert-butoxycarbonylamino)-3-methoxy-3-oxo-propyl]-iodo-zinc (3.60 g, crude) was obtained as colorless liquid, which was used to next step directly. To a solution of compound H-8 (390 mg, 1.00 mmol, 1.00 eq) in DMF (5 mL) was added [(2R)-2-(tert-butoxycarbonylamino)-3-methoxy-3-oxo-propyl]-iodo-zinc (1.19 g, 3.02 mmol, 3.00 eq), Pd₂(dba)₃ (93.0 mg, 101 μmol, 0.01 eq) and SPhos (83.0 mg, 202 μmol, 0.02 eq) under N₂ atmosphere, and the mixture was stirred at 75-80° C. for 3 hrs. The resulting product was filtered to remove the insoluble. And the reaction mixture was concentrated under reduced pressure to give a residue. The residue was purified by prep-HPLC (FA condition; column: Phenomenex luna C18 250*50 mm*10 um; mobile phase: [water (0.225% FA)-ACN]; B %: 4%-34%, 20 min). Compound H-10 (180 mg, 352 μmol, 35.1% yield) was obtained as a brown solid. LC-MS (M+H)⁻ 511.4. ¹H NMR (400 MHz, CDCl₃) δ 8.21 (br d, J=8.4 Hz, 1H), 7.66 (t, J=8.0 Hz, 1H), 7.56 (dd, J₁=8.0 Hz, J₂=2.0 Hz, 1H), 7.40-7.48 (m, 3H), 7.32 (t, J=5.6 Hz, 1H), 6.00 (d, J=8.0 Hz, 1H), 5.09 (d, J=6.8 Hz, 1H), 4.72 (d, J=7.6 Hz, 1H), 4.17-4.36 (m, 2H), 3.65 (s, 3H), 3.32-3.52 (m, 4H), 2.85 (d, J=3.6 Hz, 6H), 1.35-1.50 (m, 9H).

Step 7: To a solution of compound H-10 (80.0 mg, 157 μmol, 1.00 eq) in DCM (2.00 mL) was added HCl/dioxane (4.00 M, 5.00 mL, 128 eq). The mixture was stirred at 20-25° C. for 2 hrs. The reaction mixture was concentrated under reduced pressure to give a residue. Compound H-11 (75.0 mg, crude, HCl) was obtained as a brown solid. LC-MS (M+H)⁻ 411.2. ¹H NMR (400 MHz, DMSO-d₆) δ 10.64-10.74 (m, 1H), 8.75 (s, 3H), 8.22 (d, J=8.8 Hz, 1H), 7.94 (d, J=8.0 Hz, 1H), 7.58-7.75 (m, 3H), 7.38-7.50 (m, 2H), 5.97 (dd, J₁=8.0 Hz, J₂=3.6 Hz, 1H), 4.18-4.34 (m, 2H), 4.04-4.16 (m, 1H), 3.58 (d, J=8.4 Hz, 5H), 3.42 (d, J=7.2 Hz, 2H), 2.81 (d, J=1.6 Hz, 6H).

Step 8: To a solution of compound H-11 (65.0 mg, 145 μmol, 1.00 eq, HCl) in pyridine (2.00 mL) was added compound H-12 (32.0 mg, 202 μmol, 1.39 eq) and EDCI (58.0 mg, 302 μmol, 2.08 eq). The mixture was stirred at 20-25° C. for 1 hr. The reaction mixture was concentrated under reduced pressure to give a residue. Compound H-13 (110 mg, crude) was obtained as yellow oil. LC-MS (M+H)⁺ 551.3.

Step 9: To a solution of compound H-13 (100 mg, 182 μmol, 1.00 eq) in dioxane (1.00 mL) was added HCl (4.00 M, 5.00 mL, 110 eq). The mixture was stirred at 55-60° C. for 1 hr. The reaction mixture was concentrated under reduced pressure to give a residue. The residue was purified by prep-HPLC (FA condition; column: Phenomenex Luna C18 150*25 mm*10 um; mobile phase: [water (FA)-ACN]; B %: 1%-29%, 10 min). Compound 119 (18.65 mg, 33.5 μmol, 18.4% yield, 96.3% purity) was obtained as a white solid. LC-MS (M+H)⁺ 537.2. ¹H NMR (400 MHz, DMSO-d₆) δ 9.18 (d, J=7.6 Hz, 1H), 8.25 (d, J=8.6 Hz, 1H), 7.87 (d, J=8.0 Hz, 1H), 7.69 (t, J=8.0 Hz, 1H), 7.35-7.53 (m, 6H), 7.12 (t, J=8.0 Hz, 2H), 5.86 (dd, J₁=8.0 Hz, J₂=5.2 Hz, 1H), 4.65-4.76 (m, 1H), 3.84-3.89 (m, 2H), 3.72 (d, J=3.6 Hz, 2H), 2.22 (d, J=1.2 Hz, 6H).

### Example 21: Synthesis of Compound 287

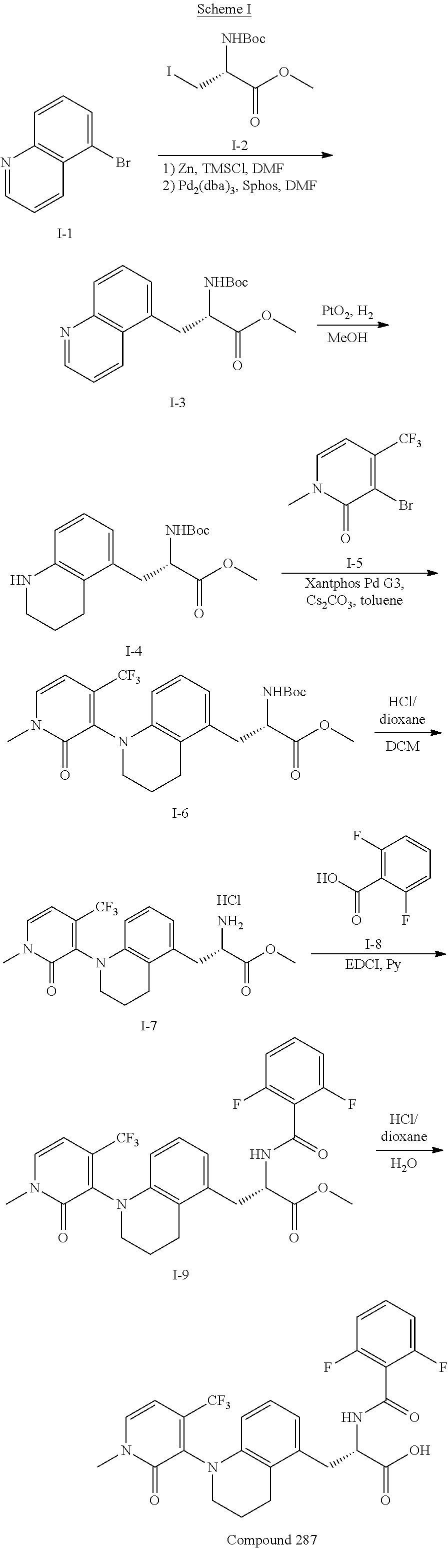

Step 1: Zn (26.8 g, 410 mmol, 3.00 eq) was added to a three necked flask, and heated at 110° C. under vacuum for 10 mins, then cooled to 20° C. To the flask was added a solution of TMSCl (1.49 g, 13.7 mmol, 1.74 mL, 0.100 eq) in DMF (200 mL) and stirred at 20° C. for 30 min. And the supernatant liquor was removed by syringe. Then a mixture of methyl compound I-2 (45.0 g, 137 mmol, 1.00 eq) in DMF (200 mL) was added to the precipitate. And the resulting mixture was stirred at 20° C. for 1 hr. Compound [(2R)-2-(tert-butoxycarbonylamino)-3-methoxy-3-oxo-propyl]-iodo-zinc (53.9 g, crude) (assume 100% yield) in DMF (180 mL) was obtained as a light-yellow liquid, the mixture was used directly. A mixture of compound I-1 (8.00 g, 38.5 mmol, 1.00 eq), [(2R)-2-(tert-butoxycarbonylamino)-3-methoxy-3-oxo-propyl]-iodo-zinc (45.5 g, 115 mmol, 3.00 eq), Pd₂(dba)₃ (1.76 g, 1.92 mmol, 0.05 eq), SPhos (1.58 g, 3.85 mmol, 0.10 eq) in DMF (180 mL) was degassed and purged with N₂ for 3 times, and then the mixture was stirred at 80° C. for 10 hrs under N₂ atmosphere. The reaction mixture was filtered and washed with ethyl acetate (200 mL). The filtrate was diluted with H₂O 1000 mL and extracted with ethyl acetate 450 mL (150 mL*3). The combined organic layers were dried over Na₂SO₄, filtered and concentrated under reduced pressure to give a residue. The residue was purified by column chromatography (SiO₂, Petroleum ether/Ethyl acetate=1/1). TIC (Petroleum ether/Ethyl acetate=1/1, R_{f}=0.42). Compound I-3 (7.00 g, 21.2 mmol, 55.1% yield) was obtained as a yellow oil. LC-MS (M+H)⁺ 331.1. ¹H NMR (400 MHz, DMSO-d₆) δ 8.91 (dd, J₁=4.0 Hz, J₂=1.2 Hz, 1H), 8.50 (d, J=8.4 Hz, 1H), 7.91 (d, J=8.4 Hz, 1H), 7.65 (t, J=7.2 Hz, 1H), 7.59 (q, J=4.4 Hz, 1H), 7.49 (d, J=7.2 Hz, 1H), 7.42 (d, J=8.4 Hz, 1H), 4.35-4.21 (m, 1H), 3.60 (s, 3H), 3.58-3.50 (m, 1H), 3.31-3.28 (m, 1H), 1.27 (s, 9H).

Step 2: To a solution of methyl compound I-3 (2.00 g, 6.05 mmol, 1.00 eq) in MeOH (30 mL) was added PtO₂ (138 mg, 605 μmol, 0.10 eq) under N₂, the reaction mixture was stirred at 25° C. for 10 hrs under H₂ (15 psi). The reaction mixture was filtered and concentrated under reduced pressure to give a residue. The residue was purified by column chromatography (SiO₂, Petroleum ether/Ethyl acetate=3/1). Compound I-4 (2.00 g, 5.98 mmol, 98.8% yield) was obtained as a white oil. LC-MS (2M+H) 669.3. ¹H NMR (400 MHz, DMSO-d₆) δ 7.27 (d, J=7.6 Hz, 1H), 6.74 (t, J=7.6 Hz, 1H), 6.36-6.25 (m, 2H), 5.56 (s, 1H), 4.06-3.99 (m, 1H), 3.59 (s, 3H), 3.15-3.10 (m, 2H), 2.90-2.80 (m, 1H), 2.74-2.66 (m, 3H), 1.88-1.68 (m, 2H), 1.34 (s, 9H).

Step 3: A mixture of compound I-4 (2.00 g, 5.98 mmol, 1.00 eq), compound I-5 (2.30 g, 8.97 mmol, 1.50 eq), Cs₂CO₃ (3.90 g, 12.0 mmol, 2.00 eq), XantPhos Pd G3 (284 mg, 299 μmol, 0.0500 eq) in toluene (20 mL) was degassed and purged with N₂ for 3 times, and then the mixture was stirred at 90° C. for 5 hrs under N₂ atmosphere. The reaction mixture was filtered and washed with Ethyl acetate 100 mL through celatom. The filtrate was concentrated under reduced pressure to give a residue. The residue was purified by column chromatography (SiO₂, Petroleum ether/Ethyl acetate=1/1) and further purified by prep-TIC (SiO₂, Petroleum ether/Ethyl acetate=1/1). TLC (Petroleum ether/Ethyl acetate=1/1, R_{f}=0.20). Compound I-6 (128 mg, 251 μmol, 4.20% yield) was obtained as a yellow oil. LC-MS (M+H₂O)⁺527.3. ¹H NMR (400 MHz, DMSO-d₆) δ 7.97 (d, J=7.2 Hz, 1H), 7.35-7.31 (m, 1H), 6.80-6.67 (m, 1H), 6.57-6.40 (m, 2H), 5.86 (d, J=8.4 Hz, 1H), 4.24-4.04 (m, 1H), 3.66-3.61 (m, 3H), 3.50-3.44 (m, 4H), 3.18-3.03 (m, 1H), 2.98-2.65 (m, 4H), 2.05-1.88 (m, 2H), 1.35 (d, J=4.4 Hz, 9H).

Step 4: To a solution of compound I-6 (128 mg, 251 μmol, 1.00 eq) in DCM (0.500 mL) was added HCl/dioxane (4.00 M, 0.500 mL, 7.96 eq). The mixture was stirred at 25° C. for 0.5 hr. The reaction mixture was concentrated under reduced pressure to give a residue. Compound I-7 (112 mg, HCl, crude) was obtained as a yellow oil. LC-MS (M+H)⁻ 410.1.

Step 5: To a solution of compound I-7 (112 mg, 251 μmol, 1.00 eq, HCl), compound I-8 (39.7 mg, 251 μmol, 1.00 eq) in pyridine (1.50 mL) was added EDCI (96.3 mg, 502 μm ol, 2.00 eq). The mixture was stirred at 25° C. for 1 hr. The reaction mixture was quenched by addition sat. NH₄Cl 20 mL at 20° C., and then diluted with H₂O 20 mL and extracted with solvent Ethyl acetate 15 mL (5 mL*3). The combined organic layers were dried over Na₂SO₄, filtered and concentrated under reduced pressure to give a residue. The residue was purified by prep-TLC (SiO₂, Ethyl acetate/Petroleum ether=2/1). TLC (Ethyl acetate/Petroleum ether=2/1, R_{f}=0.61). Compound I-9 (95.0 mg, crude) was obtained as a yellow oil. LC-MS (M+H)⁺ 550.1. ¹H NMR NMR (400 MHz, DMSO-d₆) δ 9.30 (t, J=7.2 Hz, 1H), 7.98 (d, J=7.6 Hz, 1H), 7.58-7.43 (m, 1H), 7.15 (t, J=8.0 Hz, 2H), 6.79-6.74 (m, 1H), 6.57-6.49 (m, 2H), 5.90-5.87 (m, 1H), 4.66-4.54 (m, 1H), 3.66 (s, 3H), 3.51-3.45 (m, 4H), 3.17-2.85 (m, 3H), 2.82-2.65 (m, 2H), 2.12-2.01 (m, 1H), 1.99-1.91 (m, 1H). LC-MS (M+H)⁺ 550.2.

Step 6: To a solution of compound I-9 (95.0 mg, 173 μmol, 1.00 eq) in H₂O (2.0 mL) was added HCl/dioxane (4 M, 2.00 mL, 46.3 eq). The mixture was stirred at 60° C. for 1 hr. The reaction mixture was concentrated under reduced pressure to give a residue. The residue was purified by prep-HPLC (column: Welch Ultimate AQ-C18 150*30 mm*5 μm; mobile phase: [water (HCl)-ACN]; B %: 31%-61%, 10 min). Compound 287 (27.12 mg, 49.2 μmol, 28.5% yield, 97.2% purity) was obtained as a yellow solid. LC-MS (M+H)⁺ 536.1. ¹H NMR NMR (400 MHz, DMSO-d₆) δ 9.15 (t, J=8.4 Hz, 1H), 7.97 (d, J=7.2 Hz, 1H), 7.60-7.39 (m, 1H), 7.13 (t, J=7.6 Hz, 2H), 6.77-6.73 (m, 1H), 6.58-6.55 (m, 1H), 6.52-6.48 (m, 1H), 5.89-5.86 (m, 1H), 4.62-4.42 (m, 1H), 3.53-3.43 (m, 4H), 3.18-3.02 (m, 2H), 2.94-2.72 (m, 3H), 2.12-1.89 (m, 2H).

### Example 22: Synthesis of Compound 3

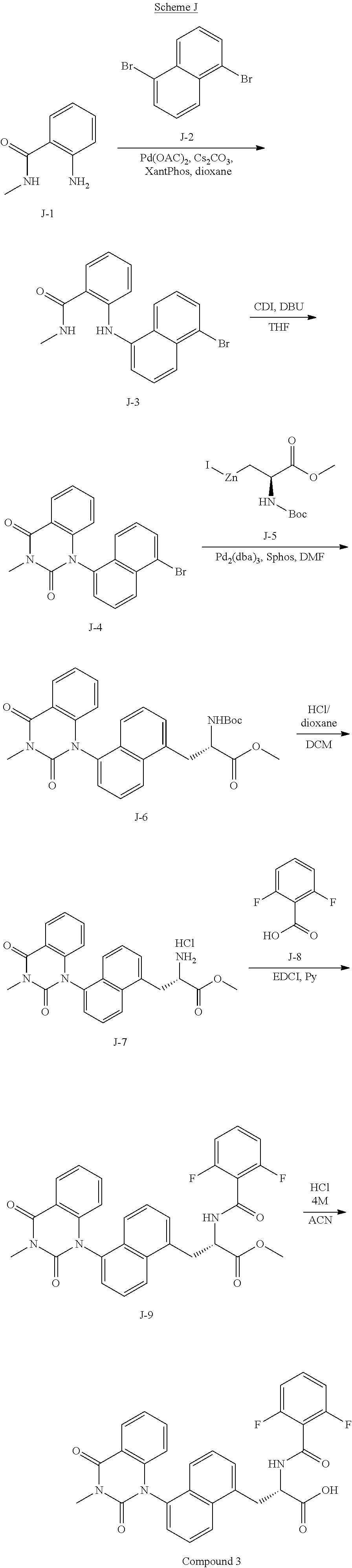

Step 1: A mixture of compound J-1 (1.00 g, 6.66 mmol, 1.00 eq), compound J-2 (2.00 g, 6.99 mmol, 1.05 eq), Pd(OAc)₂ (149 mg, 666 μmol, 0.100 eq), Xantphos (771 mg, 1.33 mmol, 0.200 eq) and Cs₂CO₃ (4.34 g, 13.3 mmol, 2.00 eq) in dioxane (10 mL) was degassed and purged with N₂ for 3 times, and then the mixture was stirred at 90° C. for 12 hrs under N₂ atmosphere. The reaction mixture was partitioned between EtOAc (50 ml*3) and H₂O 50 mL. The organic phase was separated, washed with brine (40 mL*3), dried over Na₂SO₄, filtered and concentrated under reduced pressure to give a residue. The residue was purified by Prep-TLC (Petroleum ether:Ethyl acetate=3:1, R_{f}=0.20), TLC (Petroleum ether:Ethyl acetate=3:1, R_{f}=0.20). Compound J-3 (700 mg, 1.97 mmol, 29.6% yield) was obtained as yellow solid. LC-MS (M+H)⁺ 357.2. ¹H NMR (400 MHz, DMSO-d₆) δ 10.53 (s, 1H), 8.65 (d, J=4.4 Hz, 1H), 8.09 (d, J=8.4 Hz, 1H), 7.94 (d, J=7.2 Hz, 1H), 7.86 (d, J=8.4 Hz, 1H), 7.71 (d, J=8.0 Hz, 1H), 7.66-7.60 (m, 1H), 7.58-7.54 (m, 1H), 7.52-7.46 (m, 1H), 7.34-7.28 (m, 1H), 7.16 (d, J=8.0 Hz, 1H), 6.86 (t, J=7.2 Hz, 1H), 2.80 (d, J=4.4 Hz, 3H).

Step 2: The mixture of compound J-3 (500 mg, 1.41 mmol, 1.00 eq), DBU (643 mg, 4.22 mmol, 636 μL, 3.00 eq) and CDI (685 mg, 4.22 mmol, 3.00 eq) in THF (10 mL) was stirred at 60° C. for 12 hrs. The reaction mixture was concentrated under reduced pressure to remove THF. The residue was diluted with EtOAc (20 mL*3) and extracted with H₂O 20 mL. The combined organic layers were washed with brine (20 mL*3), dried over Na₂SO₄ filtered and concentrated under reduced pressure to give a residue. The residue was purified by Prep-TLC (Petroleum ether:Ethyl acetate=2:1, R_{f}=0.30), TLC (Petroleum ether:Ethyl acetate=2:1, R_{f}=0.30). Compound J-4 (200 mg, 525 μmol, 37.3% yield) was obtained as white solid. LC-MS (M+H)⁺ 383.0. ¹H NMR (400 MHz, DMSO-d₆) δ 8.38 (d, J=8.4 Hz, 1H), 8.15 (d, J=8.0 Hz, 1H), 7.99 (d, J=7.2 Hz, 1H), 7.91 (t, J=8.0 Hz, 1H), 7.84-7.78 (m, 2H), 7.49-7.38 (m, 2H), 7.28 (t, J=7.6 Hz, 1H), 6.19 (d, J=8.4 Hz, 1H), 3.38 (s, 3H).

Step 3: To a solution of compound J-4 (200 mg, 525 μmol, 1.00 eq), Pd₂(dba)₃ (48.0 mg, 52.5 μmol, 0.100 eq) and SPhos (43.1 mg, 105 μmol, 0.200 eq) in DMF (2.00 mL) was added compound J-5 (621 mg, 1.57 mmol, 3.00 eq), then the mixture was stirred at 80° C. for 2 hrs. The reaction mixture was quenched by addition H₂O 10 mL at 0° C., and then diluted with H₂O 10 mL and extracted with EtOAc (10 mL*3). The combined organic layers were washed with brine (10 mL*3), dried over Na₂SO₄ filtered and concentrated under reduced pressure to give a residue. The residue was purified by Prep-TLC (Petroleum ether:Ethyl acetate=1:1, R_{f}=0.30), TLC (Petroleum ether:Ethyl acetate=1:1, R_{f}=0.30). Compound J-6 (160 mg, 318 μmol, 60.6% yield) was obtained as white solid. LC-MS (M-99)-404.2. ¹H NMR (400 MHz, DMSO-d₆) δ 8.30 (d, J=8.8 Hz, 1H), 8.14 (dd, J₁=1.2 Hz, J₂=8.0 Hz, 1H), 7.86-7.75 (m, 1H), 7.73-7.64 (m, 1H), 7.59 (d, J=8.4 Hz, 1H), 7.55-7.34 (m, 3H), 7.27 (t, J=7.6 Hz, 1H), 6.15 (dd, J₁=8.4 Hz, J₂=14.4 Hz, 1H), 4.38 (dd, J₁=4.8 Hz, J₂=9.6 Hz, 1H), 4.03 (q, J=7.2 Hz, 1H), 4.10-3.96 (m, 1H), 3.66-3.60 (m, 3H), 3.38 (d, J=1.2 Hz, 3H), 1.39-1.27 (m, 9H).

Step 4: To a solution of compound J-6 (160 mg, 318 μmol, 1.00 eq) in HCl/dioxane (4 M, 4.00 mL) and DCM (4.00 mL), and the mixture was stirred at 25° C. for 2 hrs. The mixture was concentrated to give a residue. Compound J-7 (160 mg, crude, HCl) was obtained as white solid and used into the next step without further purification. LC-MS (M+H)⁺ 404.2.

Step 5: A solution of compound J-7 (150 mg, 341 μmol, 1.00 eq, HCl), compound J-8 (64.7 mg, 409 mol, 1.20 eq) and EDCI (131 mg, 682 mol, 2.00 eq) in pyridine (4.00 mL) was stirred at 25° C. for 2 hrs. The mixture was concentrated to give a residue. Compound J-9 (150 mg, crude) was obtained as yellow solid and used into the next step without further purification. LC-MS (M+H)⁺ 544.2.

Step 6: A solution of compound J-9 (150 mg, 276 μmol μmol μmol, 1.00 eq) in HCl (4 M, 5.00 mL, 72.5 eq) was stirred at 60° C. for 2 hrs. The mixture was concentrated to give a residue. The residue was purified by Prep-HPLC (column: Phenomenex Luna C 18 100*30 mm*5 um; mobile phase: [water (10 mM NH₄HCO₃)-ACN]; B %: 18%-48%, 8 mins). Compound 3 (60.92 mg, 114 μmol, 41.2% yield, 98.9% purity) was obtained as off-white solid. LC-MS (M+H)⁺ 530.2. ¹H NMR (400 MHz, DMSO-d₆) δ 8.93 (d, J=3.2 Hz, 1H), 8.51-8.38 (m, 1H), 8.14 (d, J=8.0 Hz, 1H), 7.84-7.74 (m, 1H), 7.67 (d, J=2.4 Hz, 1H), 7.56 (d, J=8.4 Hz, 1H), 7.53-7.42 (m, 3H), 7.37 (t, J=7.8 Hz, 1H), 7.30-7.23 (m, 1H), 7.11 (t, J=7.6 Hz, 2H), 6.27-6.11 (m, 1H), 4.76-4.61 (m, 1H), 3.78 (d, J=4.4 Hz, 1H), 3.45-3.41 (m, 1H), 3.38 (d, J=2.4 Hz, 3H).

### Example 23: Synthesis of Compound 4

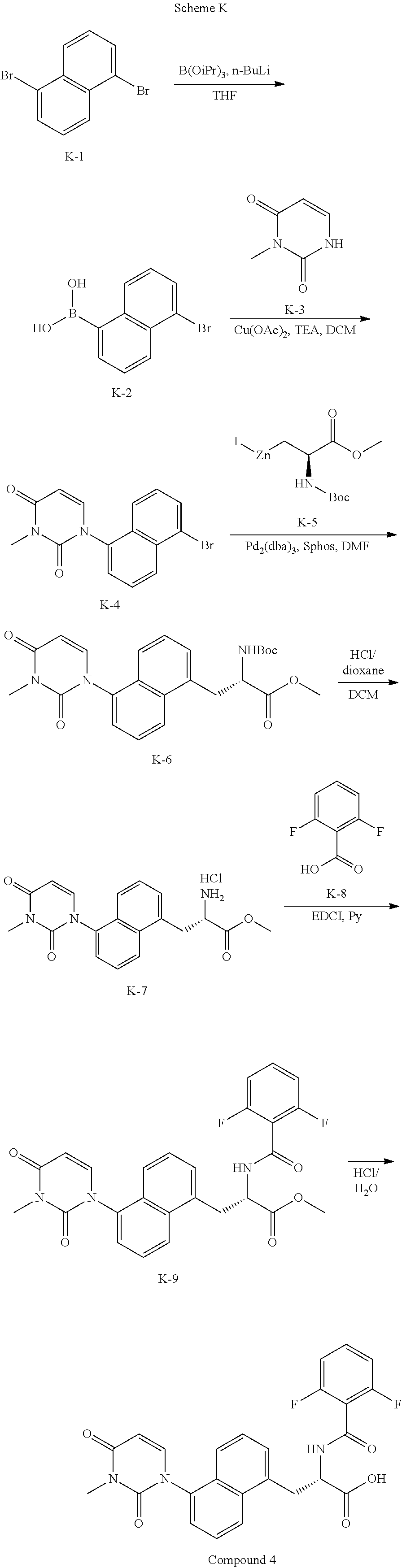

Step 1: To a solution of compound K-1 (2.00 g, 6.99 mmol, 1.00 eq) in THF (20 mL) was added dropwise n-BuLi (2.50 M, 3.08 mL, 1.10 eq) at −75° C. under N₂, and stirred at −75° C. for 1.00 hr. Triisopropyl borate (1.31 g, 6.99 mmol, 1.61 mL, 1.00 eq) was added dropwise at −75° C., and stirred at 25° C. for 2 hrs. The mixture was diluted with HCl (2.00 M, 10 mL) and H₂O (20 mL), and stirred at 25° C. for 0.5 hr. Then the mixture was extracted with DCM (20 mL*3). The combined organic layers were dried over Na₂SO₄, filtered and concentrated under reduced pressure to give a residue. The crude product was purified by re-crystallization from DCM (8.00 mL) at 20° C. Compound K-2 (1.00 g, 3.99 mmol, 57.0% yield) was obtained as a yellow solid.

Step 2: To a mixture of compound K-2 (700 mg, 2.79 mmol, 1.00 eq) and compound K-3 (528 mg, 4.19 mmol, 1.50 eq) in DCM (8.00 mL) was added Cu(OAc)₂ (507 mg, 2.79 mmol, 1.00 eq) and TEA (845 mg, 8.37 mmol, 1.17 mL, 3.00 eq), then the mixture was stirred at 20° C. for 12 hrs. The reaction mixture was quenched by addition water 20 mL and then diluted with DCM (30 mL) and extracted with DCM (10 mL*3). The combined organic layers were washed with brine (20 mL*4), dried over Na₂SO₄ filtered and concentrated under reduced pressure to give a residue. The crude product was purified by reverse-phase HPLC (0.1% TFA condition). Compound K-4 (60.0 mg, 181 μmol, 6.49% yield) was obtained as a black solid. LC-MS (M+H)⁺ 333.3. ¹H NMR (400 MHz, DMSO-d₆) δ 8.31 (d, J=8.4 Hz, 1H), 8.01-7.99 (m, 1H), 7.84-7.74 (m, 4H), 7.51 (dd, J₁=7.6 Hz, J₂=8.4 Hz, 1H), 5.89 (d, J=8.0 Hz, 1H), 3.23 (s, 3H).

Step 3: To a mixture of compound K-4 (60.0 mg, 181 μmol, 1.00 eq), Sphos (14.9 mg, 36.2 μmol, 0.200 eq) and Pd₂(dba)₃ (16.6 mg, 18.1 μmol, 0.100 eq) in DMF (3.00 mL) was added compound K-5 (214 mg, 543 μmol, 3.00 eq) in DMF (1.00 mL) was stirred at 80° C. for 12 hrs. The reaction mixture was quenched by addition water 20 mL and then diluted with ethyl acetate 60 mL and extracted with ethyl acetate (20 mL*3). The combined organic layers were washed with brine (20 mL*4), dried over Na₂SO₄ filtered and concentrated under reduced pressure to give a residue. The crude product was purified by reverse-phase HPLC (0.1% HCl condition). Compound K-6 (70.0 mg, 154 μmol, 85.2% yield) was obtained as a yellow solid. LC-MS (M-99)⁺ 354.4.

Step 4: To a solution of compound K-6 (50.0 mg, 110 μmol, 1.00 eq) in HCl/dioxane (20 mL) was stirred at 20° C. for 1 hr. The reaction mixture was concentrated under reduced pressure to give a residue. Compound K-7 (37.0 mg, 94.9 μmol, 86.1% yield, HCl) was obtained as a yellow solid.

Step 5: To a mixture of compound K-7 (30.0 mg, 76.9 μmol, 1.10 eq, HCl) and compound K-8 (11.1 mg, 69.9 μmol, 1.00 eq) in pyridine (2.00 mL) was added EDCI (26.8 mg, 140 μmol, 2.00 eq), then the reaction was stirred at 20° C. for 2 hrs. The reaction mixture was concentrated under reduced pressure to give a residue. The residue was quenched by addition water 5 mL and then diluted with DCM 15 mL and extracted with DCM (5 mL*3). The combined organic layers were washed with brine (5 mL*4), dried over Na₂SO₄ filtered and concentrated under reduced pressure to give a residue. Compound K-9 (35 mg, crude) was obtained as a yellow solid. LC-MS (M+H)+494.1.

Step 6: To a solution of compound K-9 (30.0 mg, 60.8 μmol, 1.00 eq) in HCl (2.00 mL, 12 M) and H₂O (2.00 mL) was stirred at 60° C. for 2 hrs. The reaction mixture was concentrated under reduced pressure to give a residue. The residue was purified by Prep-HPLC (column: 3_Phenomenex Luna C18 75*30.0 mm*3.00 um; mobile phase: [water (0.05% HCl)-ACN]; B %: 30%-40%, 6.00 min). Compound 4 (13.14 mg, 26.8 μmol, 44.1% yield, 97.8% purity) was obtained as a white solid. LC-MS (M+H)⁺ 480.1. ¹H NMR (400 MHz, DMSO-d₆) δ 13.14-12.96 (m, 1H), 9.27 (t, J=8.4 Hz, 1H), 8.32 (dd, J₁=3.6 Hz, J₂=8.4 Hz, 1H), 7.82-7.70 (m, 2H), 7.67 (d, J=7.2 Hz, 1H), 7.62-7.60 (m, 1H), 7.57-7.47 (m, 3H), 7.13 (t, J=8.0 Hz, 2H), 5.87 (dd, J₁=2.8 Hz, J₂=8.0 Hz, 1H), 4.77-4.68 (m, 1H), 3.82-3.72 (m, 1H), 3.40 (d, J=4.4 Hz, 1H), 3.24 (d, J=2.8 Hz, 3H).

### Example 24: Synthesis of Compound 2

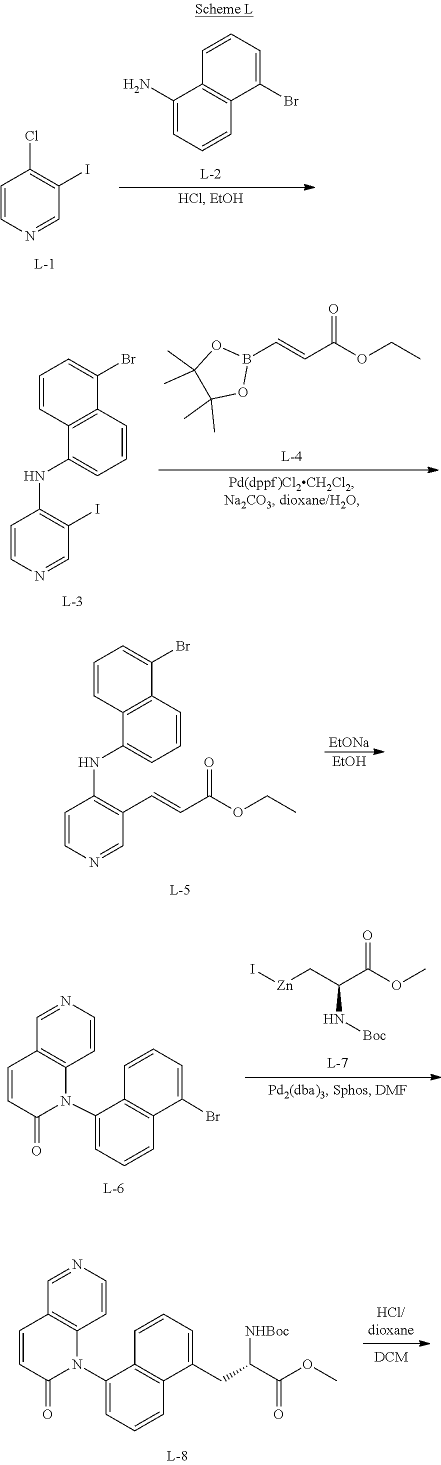

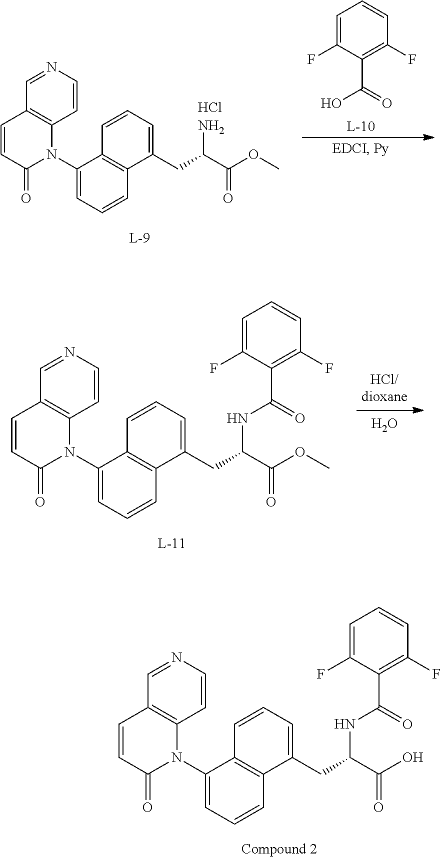

Step 1: To the mixture of compound L-1 (3.00 g, 12.5 mmol, 1.05 eq) and compound L-2 (2.12 g, 9.55 mmol, 0.800 eq) in EtOH (30 mL) was added HCl (12.0 M, 994 μL, 1.00 eq), then the mixture was stirred at 80° C. for 12 hrs. The residue was purified by prep-HPLC (column: YMC Triart C18 250*50 mm*7 um; mobile phase: [water (0.05% ammonia hydroxide v/v)-ACN]; B %: 60%-90%, 10 min). The residue was purified by prep-HPLC (column: Welch Ultimate XB-CN 250*50*10 um; mobile phase: [Hexane-EtOH]; B %: 0%-35%, 15 min). Compound L-3 (500 mg, 1.18 mmol, 9.86% yield) was obtained as a yellow solid. LC-MS (M+H)⁺ 426.9. ¹H NMR (400 MHz, CDCl₃) δ 8.70 (s, 1H), 8.29 (d, J=8.8 Hz, 1H), 8.02 (d, J=5.6 Hz, 1H), 7.91-7.83 (m, 2H), 7.69-7.61 (m, 1H), 7.54 (d, J=7.2 Hz, 1H), 7.37 (t, J=8.0 Hz, 1H), 6.65 (s, 1H), 6.33 (d, J=5.6 Hz, 1H).

Step 2: The mixture of compound L-3 (500 mg, 1.18 mmol, 1.00 eq), compound L-4 (199 mg, 882 μmol, 0.750 eq), Pd(dppf)Cl₂·CH₂Cl₂ (96.1 mg, 118 μmol, 0.100 eq) and Na₂CO₃ (249 mg, 2.35 mmol, 2.00 eq) in H₂O (12.5 mL) and dioxane (25 mL) was stirred at 90° C. for 1 hr. The reaction mixture was concentrated under reduced pressure to give a residue. The residue was purified by prep-TLC (SiO₂, Petroleum ether:Ethyl acetate=0:1). Compound L-5 (160 mg, 403 μmol, 34.2% yield) was obtained as a yellow solid. LC-MS (M+H)⁺ 397.1. ¹H NMR (400 MHz, DMSO-d₆) δ 8.62-8.48 (m, 1H), 8.29 (d, J=8.4 Hz, 1H), 8.08 (d, J=3.2 Hz, 1H), 8.03-7.92 (m, 1H), 7.86 (d, J=7.6 Hz, 2H), 7.64 (t, J=8.0 Hz, 1H), 7.50 (d, J=7.2 Hz, 1H), 7.35 (t, J=8.0 Hz, 1H), 6.84-6.67 (m, 1H), 6.82-6.61 (m, 1H), 6.63-6.51 (m, 1H), 6.39-6.27 (m, 1H), 3.49 (s, 4H).

Step 3: To a solution of compound L-5 (140 mg, 352 μmol, 1.00 eq) in EtOH (2.50 mL) was added EtONa (9.59 mg, 141 μmol, 0.400 eq), then the mixture was stirred at 70° C. for 2 hrs. The reaction mixture was concentrated under reduced pressure to give a residue. Compound 6 (160 mg, crude) was obtained as a yellow solid. LC-MS (M+H)⁺ 351.1.

Step 4: To a solution of compound L-6 (160 mg, 456 μmol, 1.00 eq), Pd₂(dba)₃ (62.6 mg, 68.3 μmol, 0.150 eq) and SPhos (56.1 mg, 137 μmol, 0.300 eq) in DMF (2.00 mL) was added compound L-7 (539 mg, 1.37 mmol, 3.00 eq) in DMF (2.25 mL), then the mixture was stirred at 80° C. for 0.5 hr. The reaction mixture was quenched by addition H₂O 20 mL at 0° C., extracted with EtOAc 9.00 mL (3.00 mL*3), and dried over Na₂SO₄, filtered and concentrated under reduced pressure to give a residue. The residue was purified by prep-TLC (SiO₂, Petroleum ether:Ethyl acetate=1:1). Compound L-8 (170 mg, 359 μmol, 78.8% yield) was obtained as a yellow solid. LC-MS (M+H)⁺ 474.3. ¹H NMR (400 MHz, CDCl₃) δ 8.89 (s, 1H), 8.41-8.29 (m, 2H), 7.97 (d, J=9.6 Hz, 1H), 7.81-7.67 (m, 1H), 7.49-7.44 (m, 1H), 7.35 (t, J=5.6 Hz, 2H), 7.25-7.16 (m, 1H), 6.96-6.90 (m, 1H), 6.37-6.24 (m, 1H), 5.25-4.99 (m, 1H), 4.82-4.72 (m, 1H), 3.83-3.73 (m, 1H), 3.49 (s, 3H), 1.50-1.33 (m, 9H).

Step 5: The mixture of compound L-8 (150 mg, 317 μmol, 1.00 eq) and HCl/dioxane (4.00 M, 2.00 mL, 25.3 eq) in DCM (2.00 mL) was stirred at 25° C. for 1 hr. The reaction mixture was concentrated under reduced pressure to give a residue. Compound L-9 (150 mg, crude, HCl) was obtained as a yellow solid. The crude product was directly used in the next step.

Step 6: The mixture of compound L-9 (100 mg, 244 μmol, 1.00 eq, HCl), compound L-10 (42.4 mg, 268 μmol, 1.10 eq) and EDCI (117 mg, 610 μmol, 2.50 eq) in pyridine (2.00 mL) was stirred at 25° C. for 1 hr. The reaction mixture was concentrated under reduced pressure to give a residue. The residue was purified by prep-HPLC (column: Waters xbridge 150*25 mm 10 um; mobile phase: [water (10 mM NH₄HCO₃)-ACN]; B %: 20%-50%, 11 min). Compound L-11 (100 mg, crude) was obtained as a yellow solid. LC-MS (M+H)⁺ 514.3.

Step 7: The mixture of compound L-11 (50.0 mg, 97.4 μmol, 1.00 eq) and HCl/dioxane (4.00 M, 1.00 mL, 41.1 eq) in H₂O (1.00 mL) was stirred at 60° C. for 1 hr. The residue was purified by prep-HPLC (column: Waters xbridge 150*25 mm 10 um; mobile phase: [water (10 mM NH₄HCO₃)-ACN]; B %: 0%-30%, 11 min). Compound 2 (20.58 mg, 40.0 μmol, 41.0% yield, 97.0% purity) was obtained as a white solid. LC-MS (M+H)⁺ 500.3. ¹H NMR (400 MHz, DMSO-d₆) δ 9.00 (s, 1H), 8.97-8.78 (m, 1H), 8.48 (dd, J₁=8.4 Hz, J₂=4.8 Hz, 1H), 8.33-8.22 (m, 2H), 7.80 (t, J=7.6 Hz, 1H), 7.61 (dd, J₁=7.2 Hz, J₂=2.4 Hz, 1H), 7.54-7.42 (m, 2H), 7.40-7.32 (m, 1H), 7.18-7.05 (m, 3H), 6.87 (dd, J₁=9.6 Hz, J₂=4.4 Hz, 1H), 6.31-6.14 (m, 1H), 4.77-4.61 (m, 1H), 3.85-3.74 (m, 1H), 3.44-3.38 (m, 1H).

### Example 25: Synthesis of Compound 89

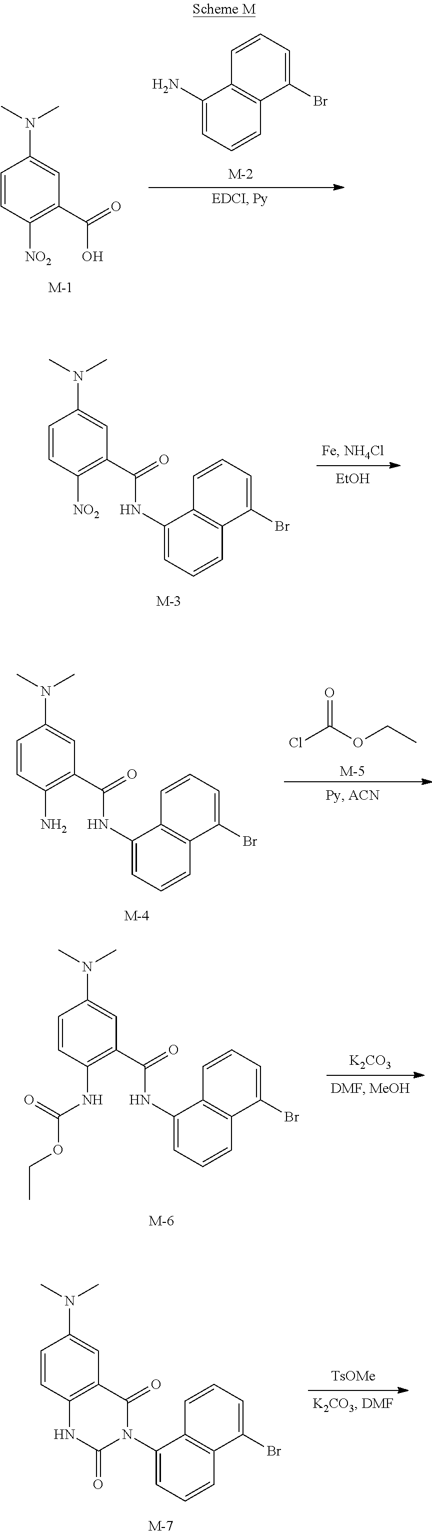

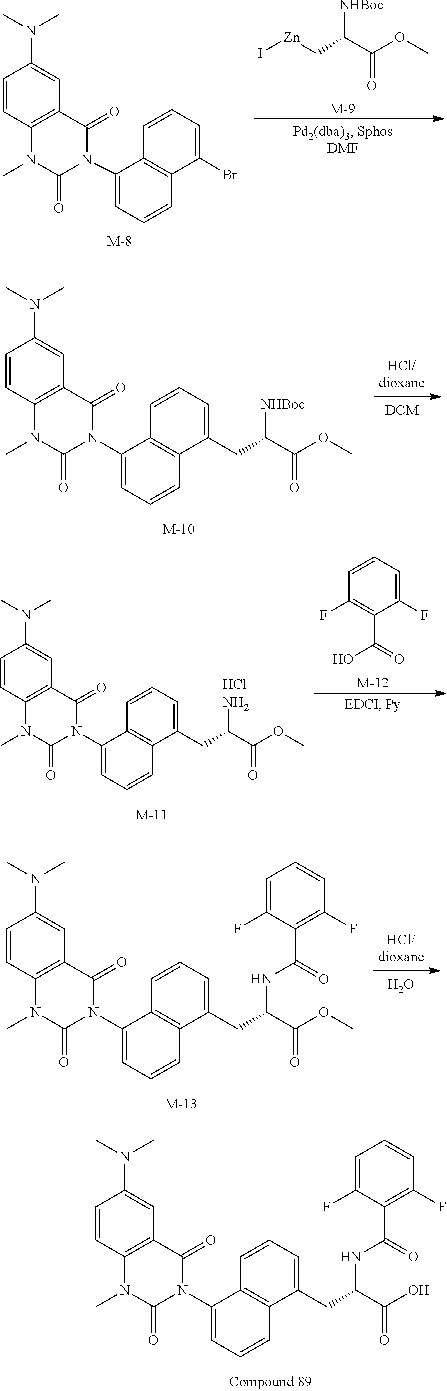

Step 1: To a solution of compound M-1 (3.00 g, 14.3 mmol, 1.00 eq), compound M-2 (3.17 g, 14.3 mmol, 1.00 eq) in pyridine (100 mL) was added EDCI (8.21 g, 42.8 mmol, 3.00 eq). The mixture was stirred at 25° C. for 1 hr. The reaction mixture was concentrated under reduced pressure to give a residue. The crude product was triturated with DCM (20 mL) at 25° C. for 30 mins. Compound M-3 (4.50 g, 10.9 mmol, 76.1% yield) was obtained as a yellow solid. LC-MS (2M+H)+829.0. ¹H NMR (400 MHz, DMSO-d₆) δ 8.23 (d, J=8.4 Hz, 1H), 8.13-8.07 (m, 2H), 7.93 (br d, J=7.6 Hz, 2H), 7.75 (t, J=8.0 Hz, 1H), 7.48 (t, J=8.0 Hz, 1H), 6.98-6.79 (m, 2H), 3.16 (s, 6H).

Step 2: A mixture of compound M-3 (4.00 g, 9.66 mmol, 1.00 eq), Fe (5.39 g, 96.6 mmol, 10.0 eq), NH₄Cl (4.13 g, 77.3 mmol, 8.00 eq) in EtOH (40 mL) was degassed and purged with N₂ for 3 times, and then the mixture was stirred at 80° C. for 3 hrs under N₂ atmosphere. The reaction mixture was filtered and concentrated under reduced pressure to give a residue. Compound M-4 (4.00 g, crude) was obtained as a yellow solid. LC-MS (M+H)⁺ 385.9.

Step 3: To a solution of compound M-4 (2.00 g, 5.20 mmol, 1.00 eq), pyridine (823 mg, 10.4 mmol, 840 μL, 2.00 eq) in ACN (30 mL) was added compound M-5 (1.32 g, 12.2 mmol, 1.16 mL, 2.34 eq) at 0° C. The mixture was stirred at 25° C. for 1 hr. The reaction mixture was quenched by addition water 100 mL at 0° C., and then extracted with Ethyl acetate 300 mL (100 mL*3). The combined organic layers were dried over Na₂SO₄, filtered and concentrated under reduced pressure to give a residue. The crude product was triturated with DCM (20 mL) at 20° C. for 30 mins. Compound M-6 (1.30 g, 2.85 mmol, 54.7% yield) was obtained as a yellow solid. LC-MS (M+H)⁺ 458.0.

Step 4: To a solution of compound M-6 (1.00 g, 2.19 mmol, 1.00 eq) in DMF (10 mL), MeOH (1.00 mL) was added K₂CO₃ (1.51 g, 11.0 mmol, 5.00 eq). The mixture was stirred at 25° C. for 3 hrs. The reaction mixture was diluted with water 50 mL and extracted with Ethyl acetate 150 mL (50 mL*3). The combined organic layers were washed with brine 300 mL (100 mL*3), dried over Na₂SO₄, filtered and concentrated under reduced pressure to give a residue. Compound M-7 (900 mg, crude) was obtained as a yellow solid. LC-MS (M+H)⁺ 410.0.

Step 5: To a solution of compound M-7 (600 mg, 1.46 mmol, 1.00 eq) in DMF (10 mL), MeOH (1.00 mL) was added TsOMe (327 mg, 1.75 mmol, 1.20 eq) and K₂CO₃ (404 mg, 2.92 mmol, 2.00 eq). The mixture was stirred at 25° C. for 3 hrs. The reaction mixture was diluted with water 50 mL and extracted with ethyl acetate 150 mL (50 mL*3). The combined organic layers were washed with brine 150 mL (50 mL*3), dried over Na₂SO₄, filtered and concentrated under reduced pressure to give a residue. Compound M-8 (700 mg, crude) was obtained as a yellow solid. LC-MS (M+H)⁺ 424.0. ¹H NMR (400 MHz, DMSO-d₆) δ 8.27 (d, J=8.4 Hz, 1H), 7.94 (d, J=7.6 Hz, 1H), 7.82-7.78 (m, 1H), 7.71-7.68 (m, 2H), 7.48-7.44 (m, 1H), 7.40-7.36 (m, 2H), 7.27 (t, J=2.8 Hz, 1H), 3.54 (s, 3H), 2.92 (s, 6H).

Step 6: To a solution of compound M-8 (700 mg, 1.65 mmol, 1.00 eq), compound M-9 (1.95 g, 4.95 mmol, 3.00 eq) in DMF (10 mL) was added Pd₂(dba)₃ (151 mg, 165 μmol, 0.100 eq) and SPhos (135 mg, 330 μmol, 0.200 eq). The mixture was stirred at 80° C. for 1 hr. The reaction mixture was diluted with water 500 mL and extracted with ethyl acetate 150 mL (50 mL*3). The combined organic layers were washed with brine 200 mL (100 mL*2), dried over Na₂SO₄, filtered and concentrated under reduced pressure to give a residue. The residue was purified by column chromatography (SiO₂, Petroleum ether/Ethyl acetate=I/O to 2/1). Compound M-10 (550 mg, 1.01 mmol, 60.9% yield) was obtained as a yellow solid. LC-MS (M+18)⁺ 564.1.

Step 7: To a solution of compound M-10 (120 mg 220 μmol, 1.00 eq) in DCM (1.00 mL) was added HCl/dioxane (4.00 M, 1.00 mL, 18.2 eq). The mixture was stirred at 25° C. for 0.5 hr. The reaction mixture was concentrated under reduced pressure to give a residue. Compound M-11 (110 mg, crude, HCl) was obtained as a white solid. LC-MS (M+H)⁺ 447.2. ¹H NMR (400 MHz, DMSO-d₆) δ 8.87 (s, 3H), 8.28 (br d, J=8.4 Hz, 1H), 7.86-7.53 (m, 6H), 7.50-7.29 (m, 2H), 4.56-4.25 (m, 4H), 3.89-3.71 (m, 2H), 3.55 (s, 3H), 3.05 (s, 6H).

Step 8: To a solution of compound M-11 (100 mg, 207 μmol, 1.00 eq, HCl), compound M-12 (32.7 mg, 207 μmol, 1.00 eq) in pyridine (1.00 mL) was added EDCI (119 mg, 621 μmol, 3.00 eq). The mixture was stirred at 25° C. for 1 hr. The reaction mixture was concentrated under reduced pressure to give a residue. Compound M-13 (110 mg, 188 μmol, 90.6% yield) was obtained as a yellow solid. LC-MS (M+H)⁺ 587.0.

Step 9: To a solution of compound M-13 (90.0 mg, 153 μmol, 1.00 eq) in H₂O (0.500 mL) was added HCl/dioxane (4.00 M, 0.500 mL, 13.0 eq). The mixture was stirred at 25° C. for 1 hr. The reaction mixture was concentrated under reduced pressure to give a residue. The residue was purified by Prep-HPLC (column: Phenomenex luna C18 150*25 mm*10 um; mobile phase: [water (FA)-ACN]; B %: 29%-59%, 15 min). Compound 89 (45.2 mg, 77.1 μmol, 50.3% yield, 97.8% purity) was obtained as a yellow solid. LC-MS (M+H)⁺ 573.0. ¹H NMR (400 MHz, DMSO-d₆) δ 13.31-12.80 (s, 1H), 9.24 (d, J=7.2 Hz, 1H), 8.26 (d, J=8.8 Hz, 1H), 7.72 (t, J=8.0 Hz, 1H), 7.58-7.44 (m, 5H), 7.42-7.34 (m, 2H), 7.26 (dd, J₁=4.0 Hz, J₂=3.2 Hz, 1H), 7.13 (t, J=8.0 Hz, 2H), 4.79-4.69 (m, 1H), 3.83-3.70 (m, 1H), 3.54 (d, J=2.4 Hz, 3H), 3.42-3.36 (m, 1H), 2.95 (s, 6H).

### Example 26: Synthesis of Compound 289

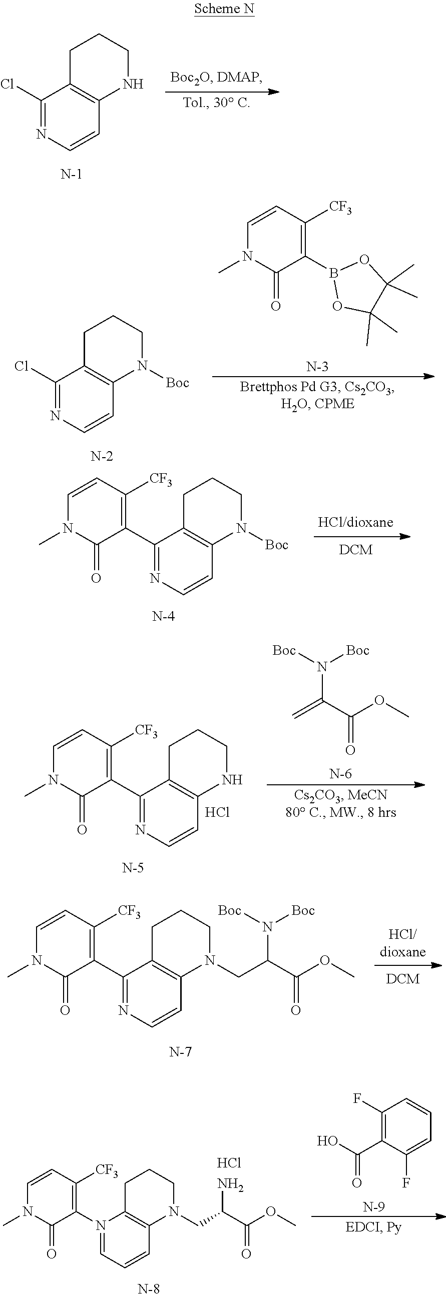

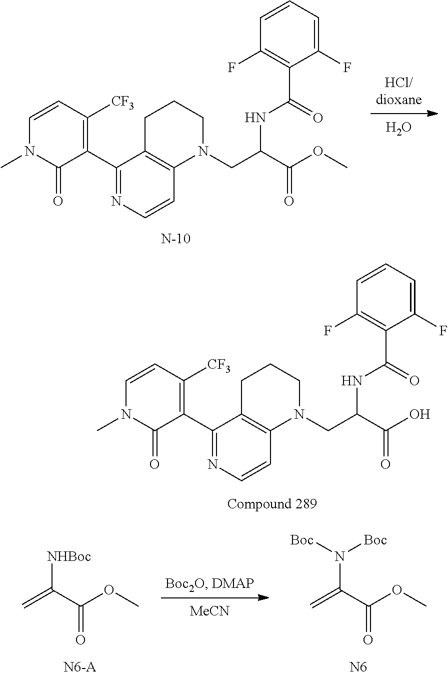

Step 1: The compound N-1 (500 mg, 2.97 mmol, 1.00 eq), Boc₂O (1.29 g, 5.93 mmol, 1.36 mL, 2.00 eq), DMAP (72.5 mg, 593 μmol, 0.200 eq) was added Tol. (5 mL). The mixture was stirred at 30° C. for 3 hrs. The reaction mixture was concentrated under reduced pressure to give a residue. Compound N-2 (800 mg, crude) was obtained as a white solid. LC-MS (M+H)⁺ 269.1. ¹H NMR (400 MHz, CDCl₃) δ 8.08 (d, J=5.6 Hz, 1H), 7.77 (d, J=6.0 Hz, 1H), 3.74 (t, J=5.6 Hz, 2H), 2.81 (t, J=6.8 Hz, 2H), 2.03-1.92 (m, 2H), 1.50 (s, 9H).

Step 2: A mixture of compound N-2 (600 mg, 2.23 mmol, 1.00 eq), compound N-3 (1.02 g, 3.35 mmol, 1.50 eq), Cs₂CO₃ (2.18 g, 6.70 mmol, 3.00 eq), BrettPhos Pd G3 (203 mg, 223 μmol, 0.100 eq) in H₂O (4.0 mL) and CPME (CAS: 5614-37-9) (8.00 mL) was degassed and purged with N₂ for 3 times, and then the mixture was stirred at 100° C. for 1 hr under N₂ atmosphere. The reaction mixture was diluted with water 30 mL and extracted with ethyl acetate 120 mL (40 mL*3). The combined organic layers were dried over Na₂SO₄, filtered and concentrated under reduced pressure to give a residue. The residue was purified by column chromatography (SiO₂, Petroleum ether:Ethyl acetate=1:0 to 0:1) (SiO₂, Petroleum ether:Ethyl acetate=0:1). Compound N-4 (200 mg, 489 mol, 21.9% yield) was obtained as a yellow oil. LC-MS (M+H)⁺ 410.1.

Step 3: To a solution of compound N-4 (200 mg, 489 μmol, 1.00 eq) in DCM (5 mL) was added HCl/dioxane (4.00 M, 3.00 mL, 24.6 eq), then the mixture was stirred at 15° C. for 22 hrs. The mixture was concentrated under reduced pressure to give a residue. Compound N-5 (150 mg, 434 μmol, 88.8% yield, HCl) was obtained as yellow solid. LC-MS (M+H)⁺ 310.1.

Step 4: To a solution of Compound N-6A (1.50 g, 7.45 mmol, 1.00 eq) in MeCN (10 mL) was added DMAP (1.37 g, 11.2 mmol, 1.50 eq) and Boc₂O (3.25 g, 14.9 mmol, 3.43 mL, 2.00 eq) at 15° C. The reaction was stirred at 15° C. for 6 hrs. The reaction mixture was concentrated under reduced pressure to remove MeCN. The residue was diluted with H₂O (50 mL) and extracted with Ethyl acetate (30 mL*3). The combined organic layers were washed with brine (30 mL*3), dried over Na₂SO₄, filtered and concentrated under reduced pressure to give a residue. The residue was purified by column chromatography (SiO₂, Petroleum ether:Ethyl acetate=100:1 to 1:1). Compound N-6 (1.40 g, 4.65 mmol, 62.3% yield) was obtained as white solid. ¹H NMR (400 MHz, CDCl₃) δ 6.35 (s, 1H), 5.65 (s, 1H), 3.80 (s, 3H), 1.47 (s, 18H).

Step 5: Compound N-6 (120 mg, 398 μmol, 1.00 eq), compound N-5 (160 mg, 462.77 μmol, 1.16 eq, HCl) and Cs₂CO₃ (779 mg, 2.39 mmol, 6.00 eq) were taken up into a microwave tube in MeCN (4.00 mL). The sealed tube was heated at 80° C. for 8 hrs under microwave. The residue was diluted with H₂O (10 mL) and extracted with ethyl acetate (10 mL*2). The combined organic layers were washed with brine (10 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; B %: 23%-53%, 10 min). Compound N-7 (0.140 g, crude) was obtained as yellow oil. LC-MS (M+H)⁺ 611.3.

Step 6: To a solution of compound N-7 (140 mg, 229 μmol, 1.00 eq) in DCM (3.00 mL) was added HCl/dioxane (4.00 M, 2.00 mL, 34.9 eq), then the mixture was stirred at 15° C. for 2 hrs. The mixture was concentrated under reduced pressure to give a residue. Compound N-8 (100 mg, 224 μmol, 97.6% yield, HCl) was obtained as white solid. LC-MS (M+H)⁺: 411.1.

Step 7: To a solution of compound N-8 (90.0 mg, 201 μmol, 1.00 eq, HCl) and compound N-9 (63.7 mg, 403 μmol, 2.00 eq) in pyridine (2.00 mL) was added EDCI (77.2 mg, 403 μmol, 2.00 eq), then the mixture was stirred at 15° C. for 2 hrs. The reaction mixture diluted with H₂O (10 mL) and extracted with ethyl acetate (10 mL*3). The combined organic layers were washed with brine mL (10 mL*3), dried over Na₂SO₄, filtered and concentrated under reduced pressure to give a residue. The residue was purified by prep-TLC (SiO₂, DCM:MeOH=10:1). Compound N-10 (85.0 mg, 154 μmol, 76.3% yield, 99.5% purity) was obtained as white solid. LC-MS (M+H)⁺ 551.1.

Step 8: To a solution of compound N-10 (18.0 mg, 32.5 μmol, 99.5% purity, 1.00 eq) in H₂O (1.00 mL) was added HCl/dioxane (4.00 M, 6.00 mL, 738 eq), the mixture was stirred at 60° C. for 1 hr. The mixture was concentrated under reduced pressure to give a residue. The residue was purified by prep-HPLC (column: Phenomenex luna C18 150*25 mm*10 um; mobile phase: [water (FA)-ACN]; B %: 8%-38%, 10 min). Compound 289 (7.29 mg, 13.6 μmol, 41.78% yield, 100% purity) was obtained as white solid. LC-MS (M+H)⁺ 537.2. ¹H NMR (400 MHz, CD₃CN) δ 7.97-7.84 (m, 2H), 7.50-7.38 (m, 1H), 7.14 (dd, J₁=26.0 Hz, J₂=7.20 Hz, 2H), 6.99 (q, J=8.4 Hz, 2H), 6.64 (d, J=7.2 Hz, 1H), 4.82-4.63 (m, 1H), 4.03-3.77 (m, 2H), 3.70-3.47 (m, 5H), 2.33 (br t, J=6.0 Hz, 2H), 1.88-1.77 (m, 2H).

### Example 27: Synthesis of Compounds 291-469

The compounds set forth in Table 4, were prepared using the synthetic procedures of any one of Examples 1-26, or analogous procedures provided herein. Alternatively, the compounds in Table 4 may be synthesized using the procedures described in Examples 28 to 70. Spectroscopic data for the compounds of Table 4 are provided in Table 13.

TABLE 13
Spectroscopic data for Compounds 290-469

| Compound | |
|---|---|
| No. | LCMS Data [M + 1] |
| 291 | 634.2 |
| 292 | 582.1 |
| 293 | 787.9 |
| 294 | 773.8 |
| 295 | 483.1 |
| 296 | 788.3 |
| 297 | 774.3 |
| 298 | 774.3 |
| 299 | 760.3 |
| 300 | 758.4 |
| 301 | 751.7 |
| 302 | 777.2 |
| 303 | 735.1 |
| 304 | 685.1 |
| 305 | 686.2 |
| 306 | 685 |
| 307 | 685 |
| 308 | 675.1 |
| 309 | 675.1 |
| 310 | 729.1 |
| 311 | 658 |
| 312 | 657.9 |
| 313 | 657.9 |
| 314 | 686 |
| 315 | 685.8 |
| 316 | 672.1 |
| 317 | 734.2 |
| 318 | 731.8 |
| 319 | 717.9 |
| 320 | 703 |
| 321 | 706.1 |
| 322 | 702.9 |
| 323 | 703 |
| 324 | 760 |
| 325 | 649 |
| 326 | 688.8 |
| 327 | 710.2 |
| 328 | 633.3 |
| 329 | 599.2 |
| 330 | 667.3 |
| 331 | 722.9 |
| 332 | 713.9 |
| 333 | 742.1 |
| 334 | 742.2 |
| 335 | 723.3 |
| 336 | 742.2 |
| 337 | 708.2 |
| 338 | 741.9 |
| 339 | 749.2 |
| 340 | 706 |
| 341 | 738 |
| 342 | 810.3 |
| 343 | 782.1 |
| 344 | 810.1 |
| 345 | 708.9 |
| 346 | 718.1 |
| 347 | 718.1 |
| 348 | 702.8 |
| 349 | 667.3 |
| 350 | 679.2 |
| 351 | 646.2 |
| 352 | 701.9 |
| 353 | 674.9 |
| 354 | 664.1 |
| 355 | 577 |
| 356 | 548.1 |
| 357 | 522.3 |
| 358 | 598.3 |
| 359 | 799.2 |
| 360 | 827.9 |
| 361 | 730.1 |
| 362 | 650.2 |
| 363 | 622.2 |
| 364 | 666.1 |
| 365 | 664.1 |
| 366 | 650 |
| 367 | 652 |
| 368 | 638.8 |
| 369 | 678.3 |
| 370 | 664.9 |
| 371 | 682 |
| 372 | 663.9 |
| 373 | 664.9 |
| 374 | 665 |
| 375 | 638.9 |
| 376 | 662.9 |
| 377 | 662.9 |
| 378 | 652.1 |
| 379 | 700.2 |
| 380 | 733.9 |
| 381 | 699.2 |
| 382 | 699.2 |
| 383 | 678 |
| 384 | 667.9 |
| 385 | 735.9 |
| 386 | 701 |
| 387 | 685.8 |
| 388 | 704.1 |
| 389 | 697.8 |
| 390 | 584.9 |
| 391 | 654.2 |
| 392 | 748 |
| 393 | 710.2 |
| 394 | 722.2 |
| 395 | 654.2 |
| 396 | 679.1 |
| 397 | 708 |
| 398 | 713.9 |
| 399 | 656.2 |
| 400 | 656.3 |
| 401 | 684.3 |
| 402 | 684.3 |
| 403 | 698.2 |
| 404 | 698.2 |
| 405 | 702.3 |
| 406 | 702.2 |
| 407 | 736.2 |
| 408 | 735.9 |
| 409 | 761.3 |
| 410 | 761.3 |
| 411 | 727.3 |
| 412 | 727.4 |
| 413 | 635.9 |
| 414 | 635.9 |
| 415 | 638.9 |
| 416 | 678.4 |
| 417 | 678.3 |
| 418 | 677.5 |
| 419 | 677.4 |
| 420 | 607.4 |
| 421 | 661.4 |
| 422 | 772.3 |
| 423 | 772.2 |
| 424 | 643.4 |
| 425 | 643.4 |
| 426 | 678.2 |
| 427 | 615.4 |
| 428 | 676.3 |
| 429 | 658.3 |
| 430 | 671.5 |
| 431 | 720 |
| 432 | 676 |
| 433 | 758.2 |
| 434 | 815.5 |
| 435 | 772.3 |
| 436 | 829.6 |
| 437 | 586.3 |
| 438 | 691.8 |
| 439 | 663.8 |
| 440 | 698.9 |
| 441 | 687.8 |
| 442 | 759.7 |
| 443 | 630.8 |
| 444 | 594.8 |
| 445 | 692.8 |
| 446 | 632.1 |
| 447 | 632.1 |
| 448 | 546.4 |
| 449 | 620.4 |
| 450 | 545.3 |
| 451 | 634.5 |
| 452 | 664.5 |
| 453 | 666.4 |
| 454 | 745.4 |
| 455 | 670.4 |
| 456 | 682.4 |
| 457 | 690.4 |
| 458 | 690.4 |
| 459 | 620.3 |
| 460 | 526.8 |
| 461 | 648.8 |
| 462 | 680.3 |
| 463 | 694.3 |
| 464 | 694.4 |
| 465 | 694.4 |
| 466 | 694.4 |
| 467 | 694.4 |
| 468 | 684.4 |
| 469 | 675.1 |

### Example 28: Synthesis of Common Intermediate (Int1)

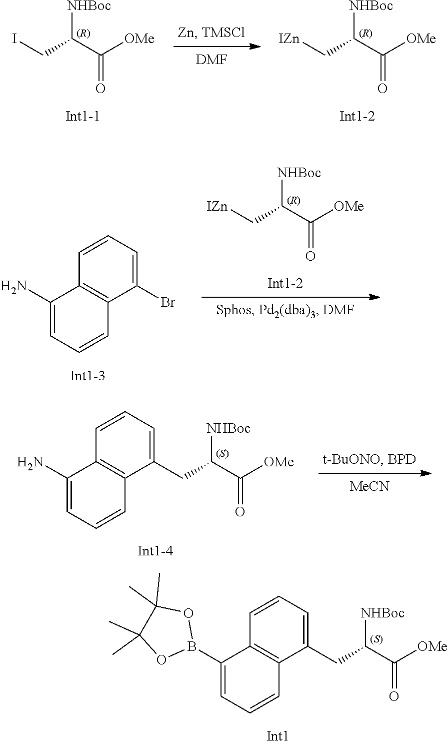

Step 1: Zn (107 g, 1.64 mol, 3.00 eq) was added to a three necked flask, and heated at 110° C. under vacuum for 10 minutes, then cooled to 25° C. To the flask was added a solution of TMSCl (11.8 g, 109 mmol, 13.8 mL, 0.200 eq) in DMF (600 mL). The mixture was stirred at 25° C. for 20 minutes. And the supernatant liquor was removed by syringe. Then a mixture of compound Int1-1 (180 g, 546 mmol, 1.00 eq) in DMF (1800 mL) was added to the precipitate, and the inner temperature was rapidly rose from 25° C. to 45° C. The resulting mixture was stirred at 20° C. for 1 hr under N₂. Compound Int1-2 (1700 g, 8 batches in total) was obtained as a gray oil, which is used for the next step directly.

Step 2: To a solution of compound Int1-3 (100 g, 450 mmol, 1.00 eq) in DMF (200 mL) was added Pd₂(dba)₃ (20.6 g, 22.5 mmol, 0.05 eq) and SPhos (36.9 g, 90.0 mmol, 0.2 eq) under N₂. Then a solution of Int1-2 (213 g, 540 mmol, 1.20 eq) in DMF (1500 mL) was added under N₂. The mixture was stirred at 90° C. for 12 hrs. The reaction mixture was filtered and concentrated under reduced pressure to give a residue. The residue was purified by flash silica gel chromatography. Compound Int1-4 (650 g, 1.89 mol, 54.1% yield, 8 batches) was obtained as a white solid. LC-MS: (M-55)⁺: 289.1. ¹H NMR: (400 MHz, CDCl₃). § 7.79 (d, J=8.4 Hz, 1H), 7.55 (d, J=8.4 Hz, 1H), 7.42-7.33 (m, 2H), 7.28 (s, 1H), 6.82 (d, J=7.2 Hz, 1H), 5.06 (d, J=7.6 Hz, 1H), 4.77-4.68 (m, 1H), 3.71 (d, J=2.4 Hz, 3H), 3.62-3.39 (m, 2H), 1.51-1.31 (m, 9H).

Step 3: To a solution of BPD (147 g, 580 mmol, 2.00 eq) in MeCN (1200 mL) was added t-BuONO (89.8 g, 871 mmol, 103 mL, 3.00 eq) at 20° C. under N₂, the mixture was warmed to 50° C. Then a solution of compound Int1-4 (100 g, 290 mmol, 1.00 eq) in MeCN (300 mL) was added dropwise at 50° C. The mixture was stirred at 50° C. for 2 hrs. The reaction mixture was filtered and concentrated under reduced pressure to give a residue. The residue was purified by reverse-phase HPLC (neutral condition, mobile phase: acetonitrile-water (80:20)). Int1 (100 g, 210 mmol, 96.0% purity) was obtained as a yellow solid. LC-MS: (M-99)⁺: 356.0. ¹H NMR: (400 MHz, CDCl₃) δ 8.73 (d, J=8.8 Hz, 1H), 8.22 (d, J=8.8 Hz, 1H), 8.12-8.07 (m, 1H), 7.59-7.52 (m, 1H), 7.48-7.42 (m, 1H), 7.30-7.28 (m, 1H), 5.08-4.95 (m, 1H), 4.76-4.65 (m, 1H), 3.66-3.46 (m, 5H), 1.48-1.39 (m, 21H).

### Example 29: General Scheme 1A

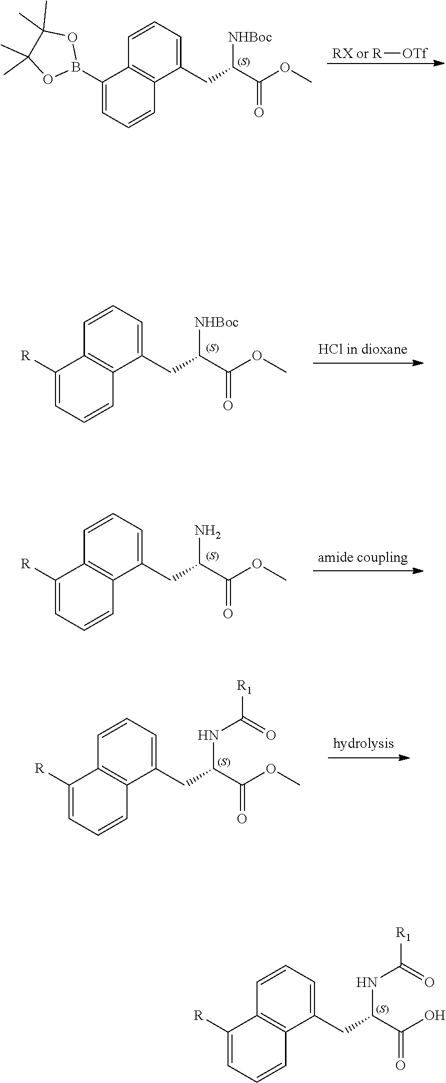

The proceeding compounds were synthesized according to the procedures described in General Scheme 1A using Int1 as an intermediate.

### Synthesis of Compound 470

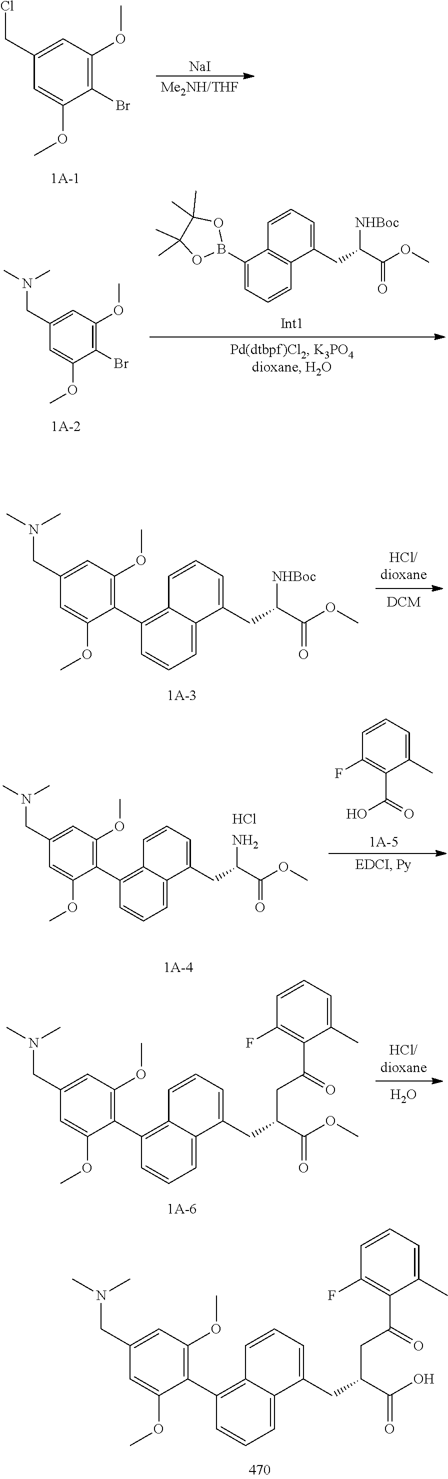

Step 1: To a solution of compound 1A-1 (250 mg, 941 μmol, 1.00 eq) was added Me₂NH/THF (2.00 M, 3.00 mL, 6.37 eq) and NaI (141 mg, 941 μmol, 1.00 eq). The mixture was stirred at 65° C. for 2 hrs. The reaction mixture was concentrated under reduced pressure to give a residue. The residue was diluted with H₂O 30.0 mL and extracted with ethyl acetate 45.0 mL (15.0 mL*3). The combined organic layers were washed with brine 10.0 mL, dried over Na 2SO₄, filtered and concentrated under reduced pressure to give a residue. The residue was purified by Prep-TLC (SiO₂, Dichloromethane:Methanol=10:1, R_{f}=0.4). Compound 1A-2 (150 mg, 547 μmol, 58.1% yield, 100% purity) was obtained as yellow oil. LC-MS: (M+H)⁺: 274.2. ¹H NMR: (400 MHz, CDCl₃) δ 6.58 (s, 2H), 3.91 (s, 6H), 3.40 (s, 2H), 2.27 (s, 6H).

Step 2: A mixture of compound 1A-2 (130 mg, 474 μmol, 1.00 eq), compound Int1 (235 mg, 474 μmol, 1.00 eq), Pd(dtbpf)Cl₂ (30.9 mg, 47.4 μmol, 0.100 eq), K₃PO₄ (302 mg, 1.42 mmol, 3.00 eq) in dioxane (4.00 mL) and H₂O (2.00 mL) was degassed and purged with N₂ for 3 times. The reaction mixture was stirred at 25° C. for 2 hrs under N₂ atmosphere. The reaction mixture was filtered to remove the insoluble, then the filter cake was washed by ethyl acetate 30.0 mL (10.0 mL*3). The filter liquor was diluted with H₂O 30.0 mL and extracted with ethyl acetate 45.0 mL (15.0 mL*3). The combined organic layers were washed with brine 20.0 mL, dried over Na₂SO₄, filtered, and concentrated under reduced pressure to give a residue. The residue was purified by Prep-TLC (SiO₂, Dichloromethane:Methanol=10:1, R_{f}=0.34). Compound 1A-3 (170 mg, 314 μmol, 66.2% yield, 96.5% purity) was obtained as yellow solid. LC-MS: (M+H)⁺: 523.4. ¹H NMR: (400 MHz, DMSO_d₆) δ 8.03 (d, J=8.4 Hz, 1H), 7.61-7.56 (m 1H), 7.45 (d, J=8.0 Hz, 1H), 7.36 (d, J=6.4 Hz, 1H), 7.29-7.20 (m, 3H), 6.74 (s, 2H), 4.35-4.32 (m, 1H), 3.69-3.65 (m, 3H), 3.60-3.58 (m, 1H), 3.56 (s, 3H), 3.55 (s, 3H), 3.48 (s, 2H), 3.26-3.24 (m, 1H), 2.24 (s, 6H), 1.32 (s, 9H).

Step 3: To a solution of compound 1A-3 (170 mg, 314 μmol, 1.00 eq) in DCM (2.00 mL) was added HCl/dioxane (4.00 M, 3.00 mL, 38.2 eq) at 0° C. The mixture was stirred at 25° C. for 1 hr. The reaction mixture was concentrated under reduced pressure to give a residue. Compound 1A-4 (140 mg, 286 μmol, 91.0% yield, 93.7% purity, HCl) was obtained as yellow solid. LC-MS: (M-99)⁻: 423.3.

Step 4: To a solution of compound 1A-4 (60.0 mg, 122 μmol, 1.00 eq, HCl) and compound 1A-5 (22.6 mg, 147 μmol, 1.20 eq) in Py (1.00 mL) was added EDCI (70.4 mg, 367 μmol, 3.00 eq). The mixture was stirred at 25° C. for 2 hrs. The reaction mixture was concentrated under reduced pressure to remove Py. The residue was diluted with sat. aq. NaHCO₃ (20.0 mL) and extracted with ethyl acetate (20.0 mL*3). The combined organic layers were washed with sat. aq. NH₄Cl (20.0 mL*2) and brine (20.0 mL*2), dried over Na₂SO₄, filtered, and concentrated under reduced pressure to give a residue. The residue was purified by Prep-TLC (SiO₂, Dichloromethane:Methanol=10:1, R_{f}=0.43). Compound 1A-6 (55.0 mg, 97.3 μmol, 79.4% yield, 98.9% purity) was obtained as yellow solid. LC-MS: (M+H)⁺: 559.3. ¹H NMR: (400 MHz, DMSO_d₆) δ 9.16 (d, J=8.0, 1H), 8.08 (d, J=8.8 Hz, 1H), 7.61 (t, J=7.2 Hz, 1H), 7.40 (d, J=6.8 Hz, 1H), 7.33-7.21 (m, 4H), 7.05-6.99 (m, 2H), 6.75 (d, J=2.4 Hz, 2H), 4.88-4.82 (m, 1H), 3.75-3.70 (m, 4H), 3.56 (d, J=9.2 Hz, 6H), 3.50 (s, 2H), 3.40-3.36 (m, 1H), 2.26 (s, 6H), 2.06 (s, 3H).

Step 5: To a solution of compound 1A-6 (50.0 mg, 88.5 μmol, 1.00 eq) in H₂O (2.00 mL) was added HCl/dioxane (4.00 M, 3.00 mL, 136 eq). The mixture was stirred at 60° C. for 2 hrs. The reaction mixture was concentrated under reduced pressure to give a residue. The residue was purified by Prep-HPLC (column: Welch Xtimate C18 150*25 mm*5 um; mobile phase: [water (HCl)-ACN]; B %: 10%-40%, 10 min). Compound 470 (25.52 mg, 43.5 μmol, 49.2% yield, 99.2% purity, HCl) was obtained as off-white solid. LC-MS: (M+H)⁺: 545.2. ¹H NMR: (400 MHz, DMSO_d₆) δ 12.98-12.97 (m, 1H), 10.32 (m, 1H), 9.04 (d, J=8.4 Hz, 1H), 8.14 (d, J=8.4 Hz, 1H), 7.66-7.61 (m, 1H), 7.43 (d, J=6.4 Hz, 1H), 7.32-7.20 (m, 4H), 7.07-6.98 (m, 4H), 4.83-4.75 (m, 1H), 4.33 (s, 2H), 3.76 (dd, J₁=14.4 Hz, J₂=3.2 Hz, 1H), 3.62 (d, J=13.2 Hz, 6H), 3.27 (s, 1H), 2.81 (s, 6H), 2.05 (s, 3H).

### Synthesis of Compound 471

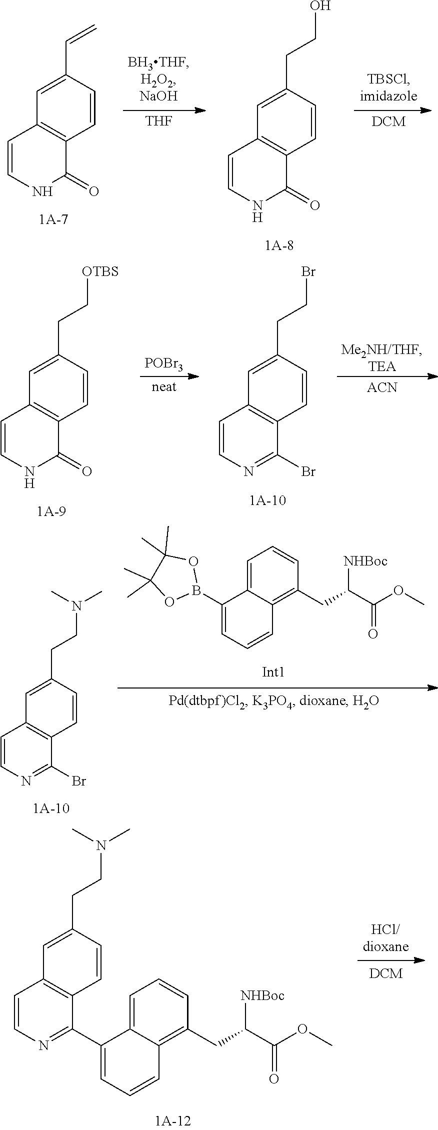

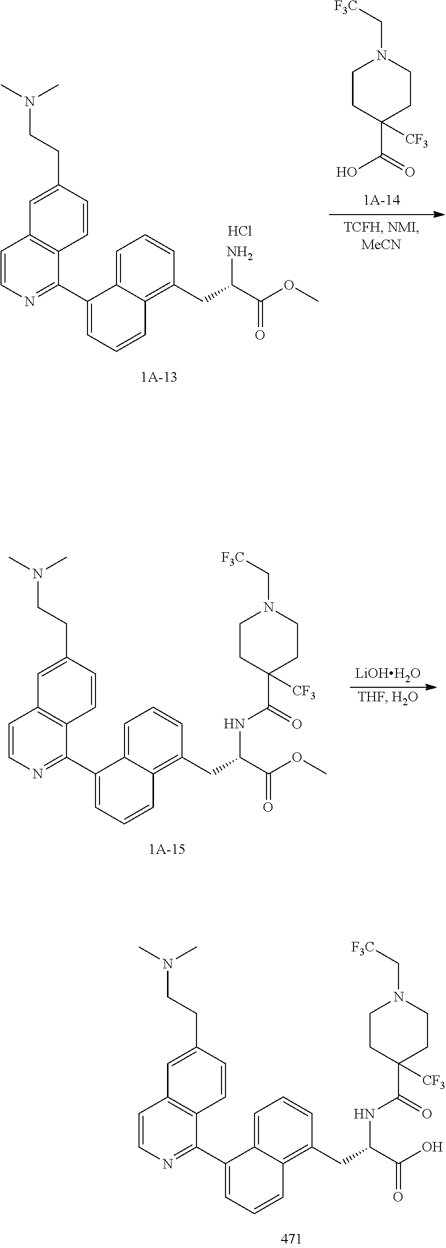

Step 1: To a stirred solution of compound 1A-7 (1.00 g, 5.84 mmol, 1.00 eq) in THF (20.0 mL) was added BH₃·THF (1.00 M, 17.5 mL, 3.00 eq) at 0° C. The reaction mixture was stirred at 25° C. for 2 hrs under N₂ atmosphere. To the reaction mixture was added H₂O₂ (1.99 g, 17.5 mmol, 1.68 mL, 30.0% purity, 3.00 eq) and NaOH (701 mg, 17.5 mmol, 3.00 eq) at 0° C. The reaction mixture was stirred at 25° C. for 2 hrs. The reaction mixture was quenched by addition sat. Na 2SO₃ 200 mL at 0° C., and then diluted with H₂O 100 mL and extracted with ethyl acetate 150 mL (50.0 mL*3). The combined organic layers were concentrated under reduced pressure to give a residue. The aqueous phase was purified by reverse MPLC (column: 330 g Flash Column Welch Ultimate XB_C18 20-40 μm; 120 A; mobile phase: [water (HCl)-ACN]; B %: 5-40% 25 min; 20% 15 min). In the organic layer, compound 1A-8 (700 mg, crude) was obtained as a yellow oil. In the aqueous layer, compound 1A-8 (420 mg, 2.22 mmol, 38.0% yield) was obtained as a white solid. LC-MS: (M+H)⁺: 190.1.

Step 2: To a solution of compound 1A-8 (400 mg, 2.11 mmol, 1.00 eq) in DCM (4.00 mL) was added TBSCl (637 mg, 4.23 mmol, 520 μL, 2.00 eq) and imidazole (287 mg, 4.23 mmol, 2.00 eq). The mixture was stirred at 25° C. for 0.5 hr. The reaction mixture was diluted with H₂O 50.0 mL and extracted with DCM 45.0 mL (15.0 mL*3). The combined organic layers were dried over Na₂SO₄, filtered, and concentrated under reduced pressure to give a residue. The residue was purified by Prep-TLC. Compound 1A-9 (550 mg, crude) was obtained as a yellow solid. LC-MS: (M+H)⁺: 304.1. ¹H NMR: (400 MHz, CDCl₃) δ 11.04 (s, 1H), 8.39-8.33 (m, 2H), 7.53 (s, 1H), 7.48 (d, J=8.4 Hz, 1H), 7.16 (br s, 1H), 3.89 (t, J=6.8 Hz, 2H), 2.96 (t, J=6.8 Hz, 2H), 0.87 (s, 9H), −0.03 (s, 6H).

Step 3: A mixture of compound 1A-9 (800 mg, 2.64 mmol, 1.00 eq), POBr₃ (1.89 g, 6.59 mmol, 670 μL, 2.50 eq) was degassed and purged with N₂ for 3 times. Then the mixture was stirred at 130° C. for 2 hrs under N₂ atmosphere. The reaction mixture was quenched by addition sat. K₂CO₃ 100 mL at 0° C. Then the mixture was diluted with H₂O 50.0 mL and extracted with ethyl acetate 45.0 ml. (15.0 ml.*3). The combined organic layers were washed concentrated under reduced pressure to give a residue. The residue was purified by Prep-TLC (SiO₂, Petroleum ether:Ethyl acetate=10:1). Compound 1A-10 (600 mg, crude) was obtained as a yellow oil. LC-MS: (M+H)⁺: 315.8. ¹H NMR: (400 MHz, DMSO-d₆) δ 8.32 (d, J=5.6 Hz, 1H), 8.22 (d, J=8.8 Hz, 1H), 7.95 (br s, 1H), 7.86 (d, J=5.6 Hz, 1H), 7.78 (dd, J₁=8.4 Hz, J₂=1.2 Hz, 1H), 3.88 (t, J=7.2 Hz, 2H), 3.41-3.37 (m, 2H).

Step 4: To a solution of compound 1A-10 (470 mg, 1.49 mmol, 1.00 eq) in ACN (5.00 mL) was added Me₂NH (2.00 M, 746 μL, 1.00 eq) and TEA (453 mg, 4.48 mmol, 623 μL, 3.00 eq). The mixture was stirred at 25° C. for 10 hrs. The reaction mixture was diluted with H₂O 50.0 mL and extracted with ethyl acetate 45.0 mL (15.0 mL*3). The combined organic layers were concentrated under reduced pressure to give a residue. The residue was purified by Prep-TLC. Compound 1A-11 (38.0 mg, crude) was obtained as a yellow oil. LC-MS: (M+H)⁺: 279.0. ¹H NMR: (400 MHz, DMSO-d₆) δ 8.24 (d, J=5.6 Hz, 1H), 8.12 (d, J=8.8 Hz, 1H), 7.88 (s, 1H), 7.83 (d, J=5.6 Hz, 1H), 7.73 (dd, J₁=8.4 Hz, J₂=1.2 Hz, 1H), 2.95 (t, J=7.2 Hz, 2H), 2.58 (d, J=7.6 Hz, 2H), 2.20 (S, 6H).

Step 5: A mixture of compound 1A-11 (40.0 mg, 143 μmol, 1.00 eq), compound Int1 (65.2 mg, 143 μmol, 1.00 eq), Pd(dtbpf)Cl₂ (9.34 mg, 14.3 μmol, 0.100 eq), K₃PO₄ (91.2 mg, 429 μmol, 3.00 eq) in dioxane (0.400 mL) and H₂O (0.100 mL) was degassed and purged with N₂ for 3 times, and then the mixture was stirred at 25° C. for 2 hrs under N₂ atmosphere. The reaction mixture was diluted with H₂O 50.0 mL and extracted with ethyl acetate 45.0 mL (15.0 mL*3). The combined organic layers were washed with dried over Na 2SO₄, filtered and concentrated under reduced pressure to give a residue. The residue was purified by Prep-TLC. Compound 1A-12 (50.0 mg, crude) was obtained as a yellow oil. LC-MS: (M+H)⁺: 528.3.

Step 6: To a solution of compound 1A-12 (50.0 mg, 94.7 μmol, 1.00 eq) in DCM (2.00 mL) was added HCl/dioxane (4.00 M, 1.00 mL, 42.2 eq) at 0° C. The mixture was stirred at 25° C. for 2 hrs. The reaction mixture was concentrated under reduced pressure to give a residue. Compound 1A-13 (40.0 mg, 86.2 μmol, 90.9% yield, HCl) was obtained as yellow solid. LC-MS: (M+H)⁺: 428.2.

Step 7: To a solution of compound 1A-13 (35.0 mg, 75.4 μmol, 1.00 eq, HCl) and compound 1A-14 (25.2 mg, 90.5 μmol, 1.20 eq) in MeCN (2.00 mL) was added NMI (21.7 mg, 264 μmol, 21.0 μL, 3.50 eq) and TCFH (25.4 mg, 90.5 μmol, 1.20 eq). The mixture was stirred at 20° C. for 1 hr. The reaction mixture was concentrated under reduced pressure to give a residue. The extracted with ethyl acetate 45.0 mL (15.0 mL*3). The combined organic layers were washed with brine 10.0 mL, dried over Na₂SO₄, filtered, and concentrated under reduced pressure to give a residue. The residue was purified by Prep-TLC. Compound 1A-15 (45.0 mg, 65.3 μmol, 86.6% yield) was obtained as yellow solid. LC-MS: (M+H)⁺: 689.2. ¹H NMR: (400 MHz, MeOD) δ 8.53 (dd, J₁=5.2 Hz, J_{2=2.0} Hz, 1H), 8.38 (dd, J₁=9.2 Hz, J₂=4.4 Hz, 1H), 7.93-7.88 (m, 2H), 7.78 (t, J=8.0 Hz, 1H), 7.61-7.57 (m, 1H), 7.43-7.35 (m, 3H), 7.26-7.21 (m, 1H), 7.13-7.06 (m, 1H), 5.13-5.06 (m, 1H), 4.59 (s, 2H), 4.00-3.93 (m, 1H), 3.78 (d, J=3.6 Hz, 3H), 3.58-3.49 (m, 1H), 3.11-3.04 (m, 2H), 2.90-2.73 (m, 4H), 2.48 (s, 6H), 2.33-2.04 (m, 4H), 1.85-1.74 (m, 2H).

Step 8: To a solution of compound 1A-15 (40.0 mg, 58.0 μmol, 1.00 eq) in THF (0.500 mL) and H₂O (0.500 mL) was added LiOH·H₂O (3.66 mg, 87.1 μmol, 1.50 eq). The mixture was stirred at 25° C. for 30 min. The reaction mixture was concentrated under reduced pressure to give a residue. The residue was purified by prep-HPLC (column: Phenomenex C18 150*25 mm*10 um; mobile phase: [water (NH₄HCO₃)-ACN]; gradient: 14%-44% B over 10 min). Compound 471 (6.93 mg, 10.2 μmol, 17.6% yield, 100% purity) was obtained as white solid. LC-MS: (M+H)⁺: 675.3. ¹H NMR: (400 MHz, MeOD) δ 8.61-8.52 (m, 2H), 7.95 (s, 1H), 7.90 (d, J=6.0 Hz, 1H), 7.74-7.68 (m, 1H), 7.51-7.37 (m, 4H), 7.20-7.13 (m, 1H), 7.02 (d, J=8.4 Hz, 1H), 4.97 (dd, J₁=10.0 Hz, J₂=4.8 Hz, 1H), 3.99-3.71 (m, 1H), 3.65-3.56 (m, 1H), 3.43-3.35 (m, 2H), 3.23-3.18 (m, 2H), 2.95-2.70 (m, 10H), 2.32-2.00 (m, 4H), 1.85-1.77 (m, 2H).

The compounds set forth in Table 14, were prepared using the synthetic procedures of Example 29, or analogous procedures as provided herein.

TABLE 14
Structure and Spectroscopic data for Compounds 472 to 550 and 567

| No. | Structure |
|---|---|
| 472 | 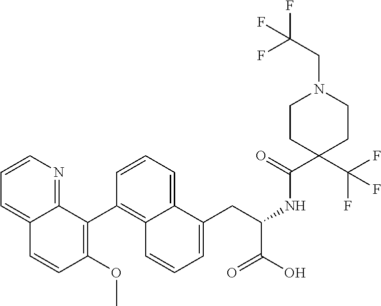 [SMILES: COc1ccc2cccnc2c1-c1cccc2c(C[C@H](NC(=O)C3(C(F)(F)F)CCN(CC(F)(F)F)CC3)C(=O)O)cccc12] |
| | m/z = 634.1 |
| 473 | 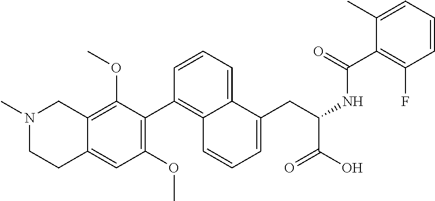 [SMILES: COc1cc2c(c(OC)c1-c1cccc3c(C[C@H](NC(=O)c4c(C)cccc4F)C(=O)O)cccc13)CN(C)CC2] |
| | m/z = 557.2 |
| 474 | 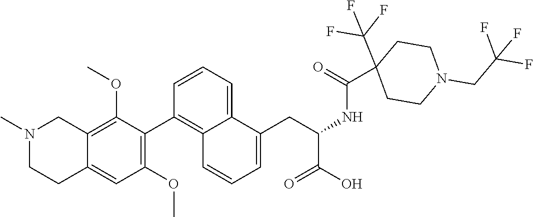 [SMILES: COc1cc2c(c(OC)c1-c1cccc3c(C[C@H](NC(=O)C4(C(F)(F)F)CCN(CC(F)(F)F)CC4)C(=O)O)cccc13)CN(C)CC2] |
| | m/z = 682.3 |
| 475 | 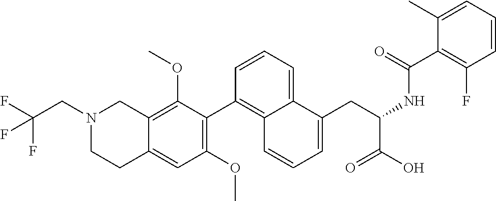 [SMILES: COc1cc2c(c(OC)c1-c1cccc3c(C[C@H](NC(=O)c4c(C)cccc4F)C(=O)O)cccc13)CN(CC(F)(F)F)CC2] |
| | m/z = 625.4 |
| 476 | 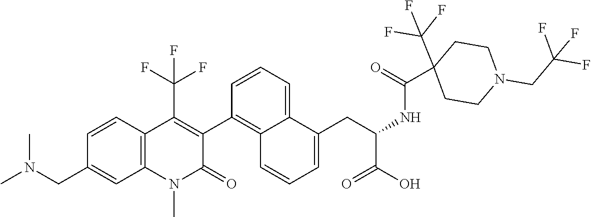 [SMILES: CN(C)Cc1ccc2c(C(F)(F)F)c(-c3cccc4c(C[C@H](NC(=O)C5(C(F)(F)F)CCN(CC(F)(F)F)CC5)C(=O)O)cccc34)c(=O)n(C)c2c1] |
| | m/z = 759.3 |
| 477 | 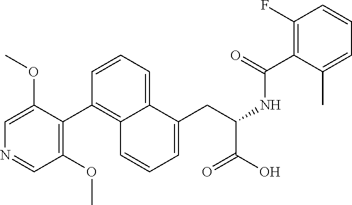 [SMILES: COc1cncc(OC)c1-c1cccc2c(C[C@H](NC(=O)c3c(C)cccc3F)C(=O)O)cccc12] |
| | m/z = 489.2 |
| 478 | 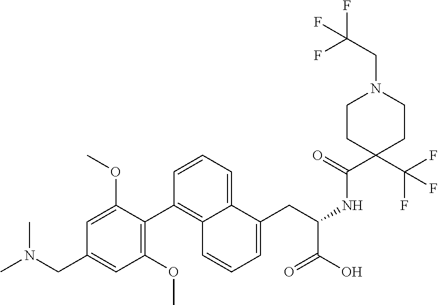 [SMILES: COc1cc(CN(C)C)cc(OC)c1-c1cccc2c(C[C@H](NC(=O)C3(C(F)(F)F)CCN(CC(F)(F)F)CC3)C(=O)O)cccc12] |
| | m/z = 670.2 |
| 479 | 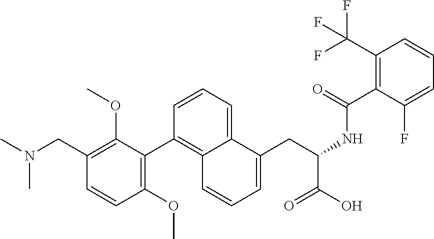 [SMILES: COc1ccc(CN(C)C)c(OC)c1-c1cccc2c(C[C@H](NC(=O)c3c(F)cccc3C(F)(F)F)C(=O)O)cccc12] |
| | m/z = 599.2 |
| 480* | 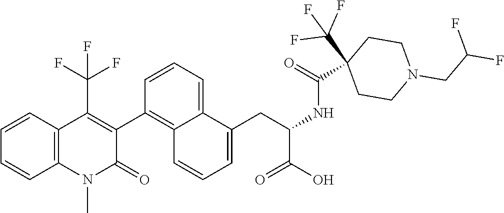 |
| | m/z = 684.2 |
| 481* | 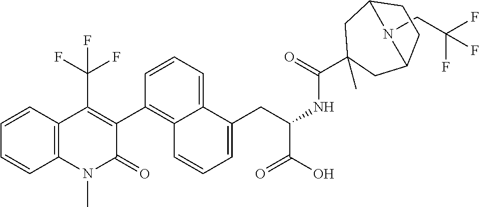 [SMILES: Cn1c(=O)c(-c2cccc3c(C[C@H](NC(=O)C4(C)CC5CCC(C4)N5CC(F)(F)F)C(=O)O)cccc23)c(C(F)(F)F)c2ccccc21] |
| | m/z = 674.4 |
| 482** | 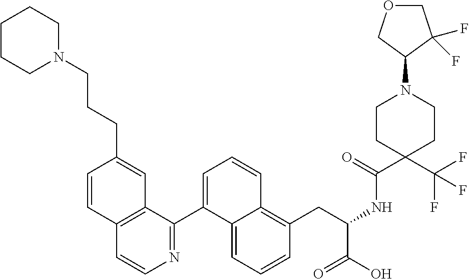 [SMILES: O=C(O)[C@H](Cc1cccc2c(-c3nccc4ccc(CCCN5CCCCC5)cc34)cccc12)NC(=O)C1(C(F)(F)F)CCN([C@H]2COCC2(F)F)CC1] |
| | m/z = 753.4 |
| 483** | 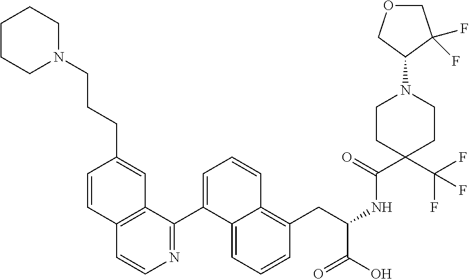 [SMILES: O=C(O)[C@H](Cc1cccc2c(-c3nccc4ccc(CCCN5CCCCC5)cc34)cccc12)NC(=O)C1(C(F)(F)F)CCN([C@@H]2COCC2(F)F)CC1] |
| | m/z = 753.4 |
| 484 | 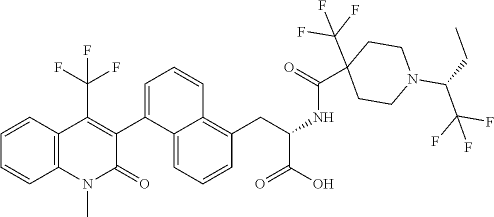 [SMILES: CC[C@@H](N1CCC(C(=O)N[C@@H](Cc2cccc3c(-c4c(C(F)(F)F)c5ccccc5n(C)c4=O)cccc23)C(=O)O)(C(F)(F)F)CC1)C(F)(F)F] |
| | m/z = 730.4 |
| 485 | 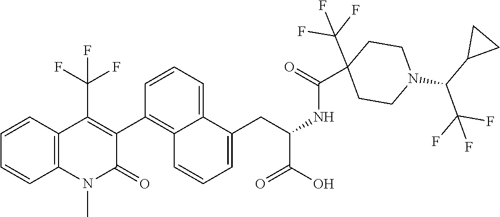 [SMILES: Cn1c(=O)c(-c2cccc3c(C[C@H](NC(=O)C4(C(F)(F)F)CCN([C@H](C5CC5)C(F)(F)F)CC4)C(=O)O)cccc23)c(C(F)(F)F)c2ccccc21] |
| | m/z = 742.2 |
| 486 | 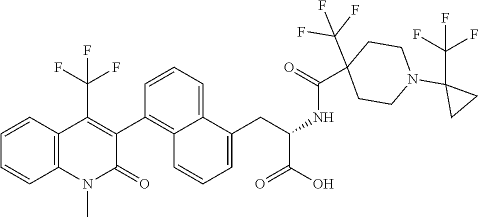 [SMILES: Cn1c(=O)c(-c2cccc3c(C[C@H](NC(=O)C4(C(F)(F)F)CCN(C5(C(F)(F)F)CC5)CC4)C(=O)O)cccc23)c(C(F)(F)F)c2ccccc21] |
| | m/z = 728.4 |
| 487 | 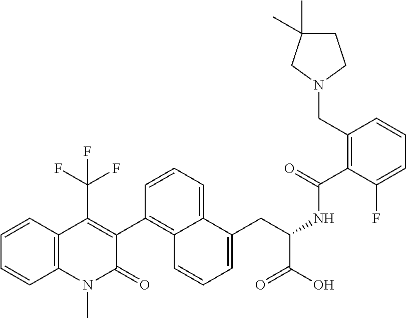 [SMILES: Cn1c(=O)c(-c2cccc3c(C[C@H](NC(=O)c4c(F)cccc4CN4CCC(C)(C)C4)C(=O)O)cccc23)c(C(F)(F)F)c2ccccc21] |
| | m/z = 673.9 |
| 488 | 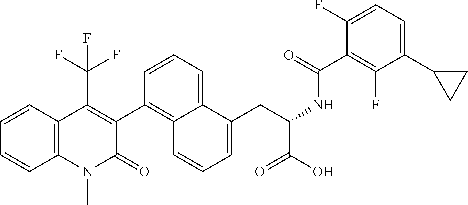 [SMILES: Cn1c(=O)c(-c2cccc3c(C[C@H](NC(=O)c4c(F)ccc(C5CC5)c4F)C(=O)O)cccc23)c(C(F)(F)F)c2ccccc21] |
| | m/z = 620.8 |
| 489 | 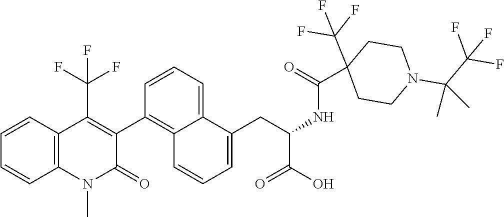 [SMILES: Cn1c(=O)c(-c2cccc3c(C[C@H](NC(=O)C4(C(F)(F)F)CCN(C(C)(C)C(F)(F)F)CC4)C(=O)O)cccc23)c(C(F)(F)F)c2ccccc21] |
| | m/z = 730.2 |
| 490 | 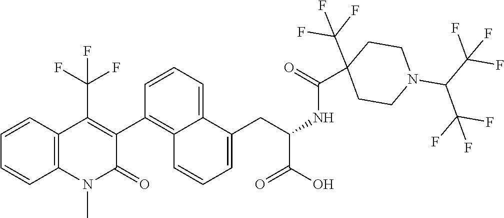 [SMILES: Cn1c(=O)c(-c2cccc3c(C[C@H](NC(=O)C4(C(F)(F)F)CCN(C(C(F)(F)F)C(F)(F)F)CC4)C(=O)O)cccc23)c(C(F)(F)F)c2ccccc21] |
| | m/z = 770.2 |
| 491 | 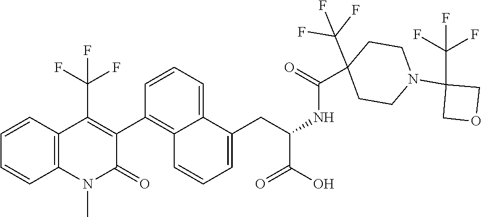 [SMILES: Cn1c(=O)c(-c2cccc3c(C[C@H](NC(=O)C4(C(F)(F)F)CCN(C5(C(F)(F)F)COC5)CC4)C(=O)O)cccc23)c(C(F)(F)F)c2ccccc21] |
| | m/z = 744.2 |
| 492 | 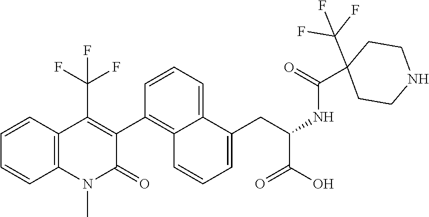 [SMILES: Cn1c(=O)c(-c2cccc3c(C[C@H](NC(=O)C4(C(F)(F)F)CCNCC4)C(=O)O)cccc23)c(C(F)(F)F)c2ccccc21] |
| | m/z = 620 |
| 493 | 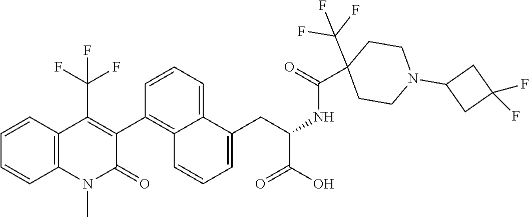 [SMILES: Cn1c(=O)c(-c2cccc3c(C[C@H](NC(=O)C4(C(F)(F)F)CCN(C5CC(F)(F)C5)CC4)C(=O)O)cccc23)c(C(F)(F)F)c2ccccc21] |
| | m/z = 710.4 |
| 494** | 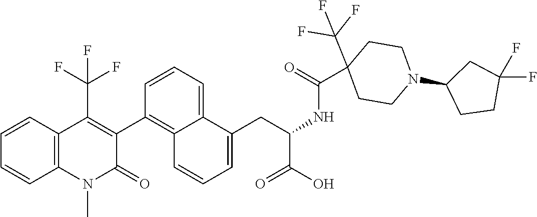 [SMILES: Cn1c(=O)c(-c2cccc3c(C[C@H](NC(=O)C4(C(F)(F)F)CCN([C@@H]5CCC(F)(F)C5)CC4)C(=O)O)cccc23)c(C(F)(F)F)c2ccccc21] |
| | m/z = 724.3 |
| 495** | 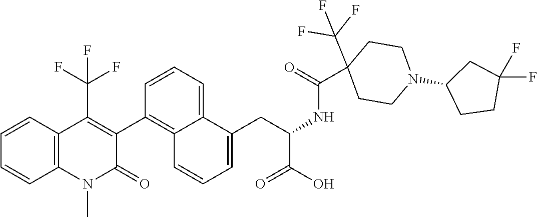 [SMILES: Cn1c(=O)c(-c2cccc3c(C[C@H](NC(=O)C4(C(F)(F)F)CCN([C@H]5CCC(F)(F)C5)CC4)C(=O)O)cccc23)c(C(F)(F)F)c2ccccc21] |
| | m/z = 724.5 |
| 496 | 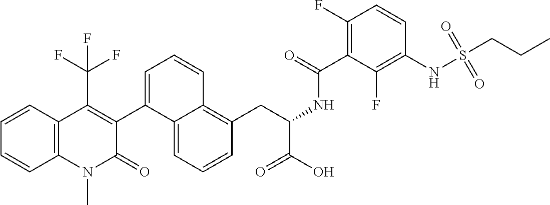 [SMILES: CCCS(=O)(=O)Nc1ccc(F)c(C(=O)N[C@@H](Cc2cccc3c(-c4c(C(F)(F)F)c5ccccc5n(C)c4=O)cccc23)C(=O)O)c1F] |
| | m/z = 701.7 |
| 497 | 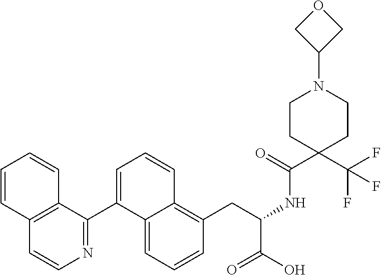 [SMILES: O=C(O)[C@H](Cc1cccc2c(-c3nccc4ccccc34)cccc12)NC(=O)C1(C(F)(F)F)CCN(C2COC2)CC1] |
| | m/z = 578.1 |
| 498 | 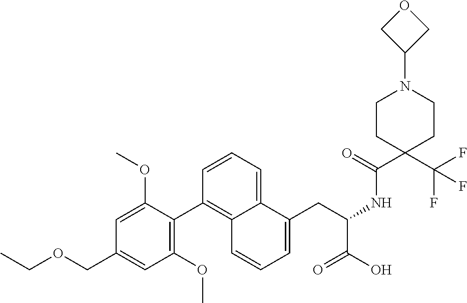 [SMILES: CCOCc1cc(OC)c(-c2cccc3c(C[C@H](NC(=O)C4(C(F)(F)F)CCN(C5COC5)CC4)C(=O)O)cccc23)c(OC)c1] |
| | m/z = 645.3 |
| 499 | 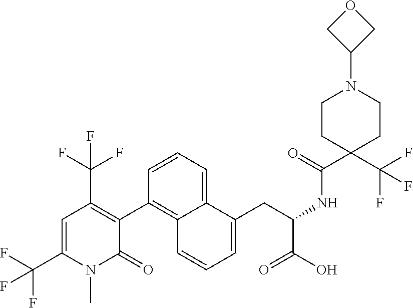 [SMILES: Cn1c(C(F)(F)F)cc(C(F)(F)F)c(-c2cccc3c(C[C@H](NC(=O)C4(C(F)(F)F)CCN(C5COC5)CC4)C(=O)O)cccc23)c1=O] |
| | m/z = 694.1 |
| 500 | 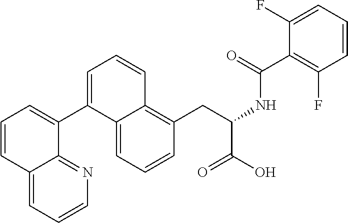 [SMILES: O=C(N[C@@H](Cc1cccc2c(-c3cccc4cccnc34)cccc12)C(=O)O)c1c(F)cccc1F] |
| | m/z = 483 |
| 501 | 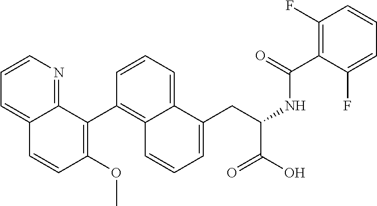 [SMILES: COc1ccc2cccnc2c1-c1cccc2c(C[C@H](NC(=O)c3c(F)cccc3F)C(=O)O)cccc12] |
| | m/z = |
| 502 | 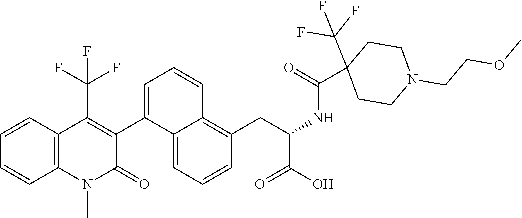 [SMILES: COCCN1CCC(C(=O)N[C@@H](Cc2cccc3c(-c4c(C(F)(F)F)c5ccccc5n(C)c4=O)cccc23)C(=O)O)(C(F)(F)F)CC1] |
| | m/z = 678.3 |
| 503 | 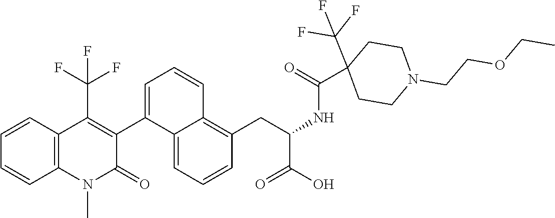 [SMILES: CCOCCN1CCC(C(=O)N[C@@H](Cc2cccc3c(-c4c(C(F)(F)F)c5ccccc5n(C)c4=O)cccc23)C(=O)O)(C(F)(F)F)CC1] |
| | m/z = 692.2 |
| 504 | 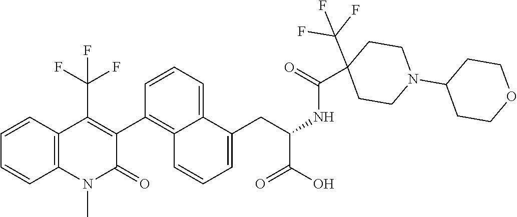 [SMILES: Cn1c(=O)c(-c2cccc3c(C[C@H](NC(=O)C4(C(F)(F)F)CCN(C5CCOCC5)CC4)C(=O)O)cccc23)c(C(F)(F)F)c2ccccc21] |
| | m/z = 704.2 |
| 505 | 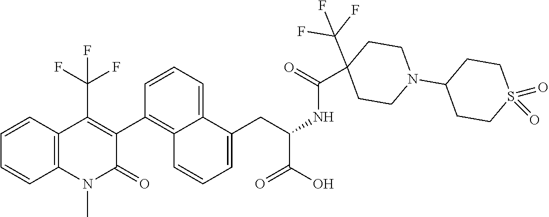 [SMILES: Cn1c(=O)c(-c2cccc3c(C[C@H](NC(=O)C4(C(F)(F)F)CCN(C5CCS(=O)(=O)CC5)CC4)C(=O)O)cccc23)c(C(F)(F)F)c2ccccc21] |
| | m/z = 752.4 |
| 506* | 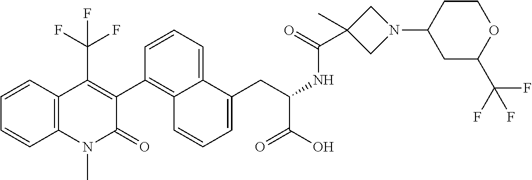 [SMILES: Cn1c(=O)c(-c2cccc3c(C[C@H](NC(=O)C4(C)CN(C5CCOC(C(F)(F)F)C5)C4)C(=O)O)cccc23)c(C(F)(F)F)c2ccccc21] |
| | m/z = 690.4 |
| 507* | 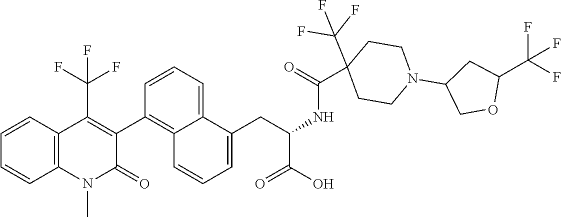 [SMILES: Cn1c(=O)c(-c2cccc3c(C[C@H](NC(=O)C4(C(F)(F)F)CCN(C5COC(C(F)(F)F)C5)CC4)C(=O)O)cccc23)c(C(F)(F)F)c2ccccc21] |
| | m/z = 758.4 |
| 508* | 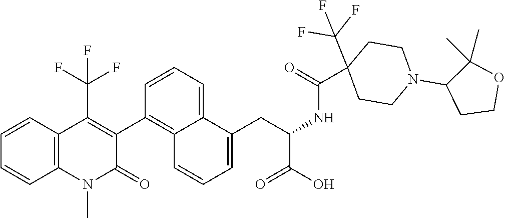 [SMILES: Cn1c(=O)c(-c2cccc3c(C[C@H](NC(=O)C4(C(F)(F)F)CCN(C5CCOC5(C)C)CC4)C(=O)O)cccc23)c(C(F)(F)F)c2ccccc21] |
| | m/z = 718.4 |
| 509* | 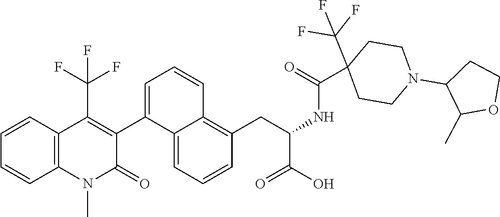 [SMILES: CC1OCCC1N1CCC(C(=O)N[C@@H](Cc2cccc3c(-c4c(C(F)(F)F)c5ccccc5n(C)c4=O)cccc23)C(=O)O)(C(F)(F)F)CC1] |
| | m/z = 704.7 |
| 510* | 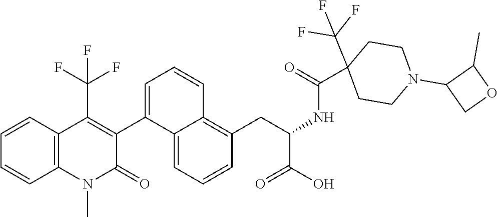 [SMILES: CC1OCC1N1CCC(C(=O)N[C@@H](Cc2cccc3c(-c4c(C(F)(F)F)c5ccccc5n(C)c4=O)cccc23)C(=O)O)(C(F)(F)F)CC1] |
| | m/z = 690.4 |
| 511* | 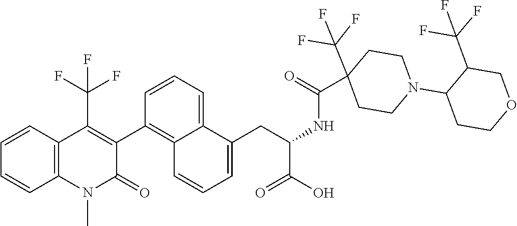 [SMILES: Cn1c(=O)c(-c2cccc3c(C[C@H](NC(=O)C4(C(F)(F)F)CCN(C5CCOCC5C(F)(F)F)CC4)C(=O)O)cccc23)c(C(F)(F)F)c2ccccc21] |
| | m/z = 772.2 |
| 512* | 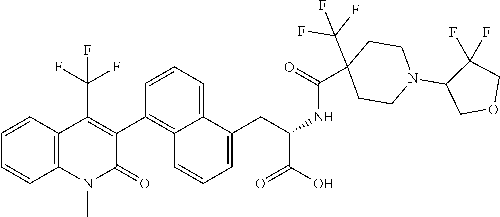 [SMILES: Cn1c(=O)c(-c2cccc3c(C[C@H](NC(=O)C4(C(F)(F)F)CCN(C5COCC5(F)F)CC4)C(=O)O)cccc23)c(C(F)(F)F)c2ccccc21] |
| | m/z = 726.2 |
| 513* | 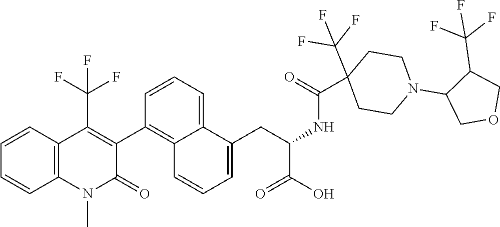 [SMILES: Cn1c(=O)c(-c2cccc3c(C[C@H](NC(=O)C4(C(F)(F)F)CCN(C5COCC5C(F)(F)F)CC4)C(=O)O)cccc23)c(C(F)(F)F)c2ccccc21] |
| | m/z = 758.2 |
| 514* | 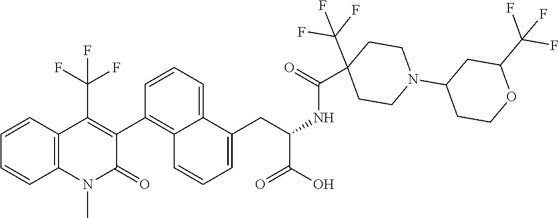 [SMILES: Cn1c(=O)c(-c2cccc3c(C[C@H](NC(=O)C4(C(F)(F)F)CCN(C5CCOC(C(F)(F)F)C5)CC4)C(=O)O)cccc23)c(C(F)(F)F)c2ccccc21] |
| | m/z = 772.2 |
| 515* | 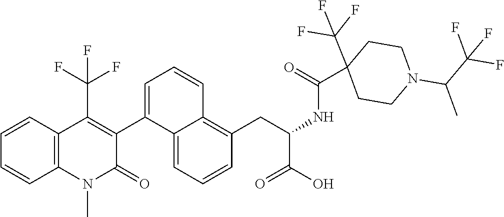 [SMILES: CC(N1CCC(C(=O)N[C@@H](Cc2cccc3c(-c4c(C(F)(F)F)c5ccccc5n(C)c4=O)cccc23)C(=O)O)(C(F)(F)F)CC1)C(F)(F)F] |
| | m/z = 716.5 |
| 516* | 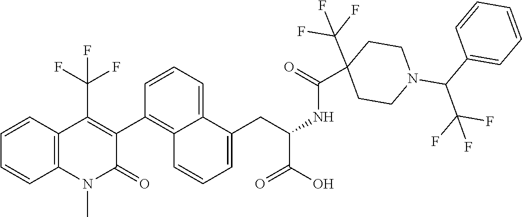 [SMILES: Cn1c(=O)c(-c2cccc3c(C[C@H](NC(=O)C4(C(F)(F)F)CCN(C(c5ccccc5)C(F)(F)F)CC4)C(=O)O)cccc23)c(C(F)(F)F)c2ccccc21] |
| | m/z = 778.2 |
| 517 | 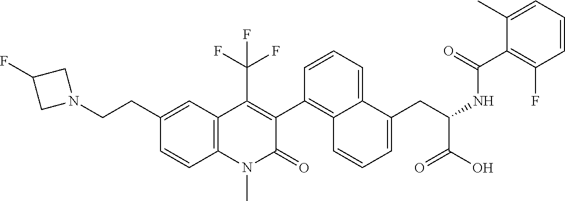 [SMILES: Cc1cccc(F)c1C(=O)N[C@@H](Cc1cccc2c(-c3c(C(F)(F)F)c4cc(CCN5CC(F)C5)ccc4n(C)c3=O)cccc12)C(=O)O] |
| | m/z = 678.4 |
| 518 | 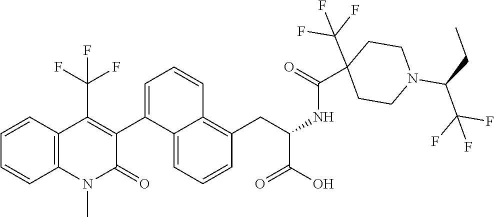 [SMILES: CC[C@H](N1CCC(C(=O)N[C@@H](Cc2cccc3c(-c4c(C(F)(F)F)c5ccccc5n(C)c4=O)cccc23)C(=O)O)(C(F)(F)F)CC1)C(F)(F)F] |
| | m/z = 730.3 |
| 519 | 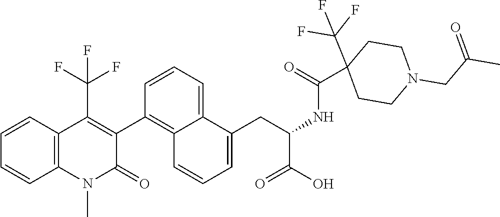 [SMILES: CC(=O)CN1CCC(C(=O)N[C@@H](Cc2cccc3c(-c4c(C(F)(F)F)c5ccccc5n(C)c4=O)cccc23)C(=O)O)(C(F)(F)F)CC1] |
| | m/z = 676.2 |
| 520* | 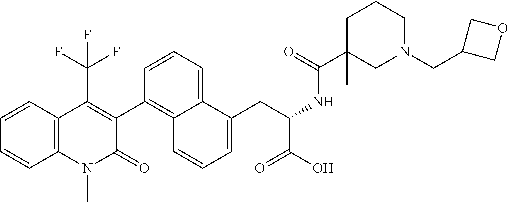 [SMILES: Cn1c(=O)c(-c2cccc3c(C[C@H](NC(=O)C4(C)CCCN(CC5COC5)C4)C(=O)O)cccc23)c(C(F)(F)F)c2ccccc21] |
| | m/z = 636.4 |
| 521 | 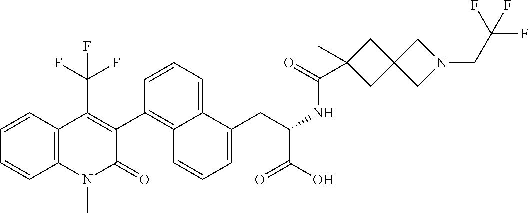 [SMILES: Cn1c(=O)c(-c2cccc3c(C[C@H](NC(=O)C4(C)CC5(CN(CC(F)(F)F)C5)C4)C(=O)O)cccc23)c(C(F)(F)F)c2ccccc21] |
| | m/z = 660.2 |
| 522 | 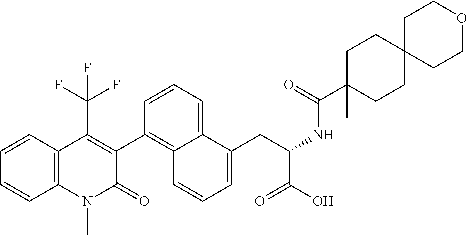 [SMILES: Cn1c(=O)c(-c2cccc3c(C[C@H](NC(=O)C4(C)CCC5(CCOCC5)CC4)C(=O)O)cccc23)c(C(F)(F)F)c2ccccc21] |
| | m/z = 635.4 |
| 523 | 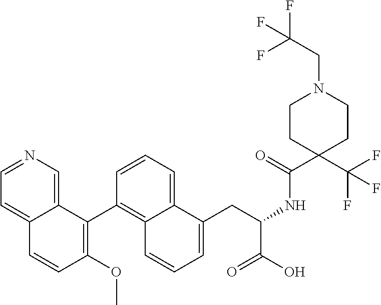 [SMILES: COc1ccc2ccncc2c1-c1cccc2c(C[C@H](NC(=O)C3(C(F)(F)F)CCN(CC(F)(F)F)CC3)C(=O)O)cccc12] |
| | m/z = 634.1 |
| 524 | 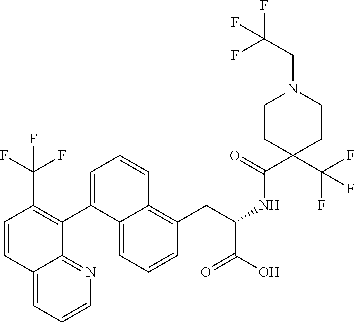 [SMILES: O=C(O)[C@H](Cc1cccc2c(-c3c(C(F)(F)F)ccc4cccnc34)cccc12)NC(=O)C1(C(F)(F)F)CCN(CC(F)(F)F)CC1] |
| | m/z = 672 |
| 525 | 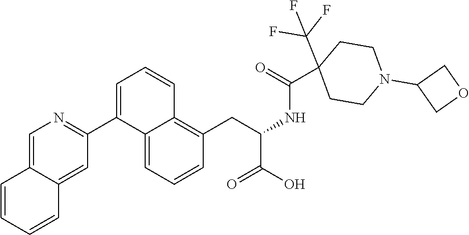 [SMILES: O=C(O)[C@H](Cc1cccc2c(-c3cc4ccccc4cn3)cccc12)NC(=O)C1(C(F)(F)F)CCN(C2COC2)CC1] |
| | m/z = 578.4 |
| 526 | 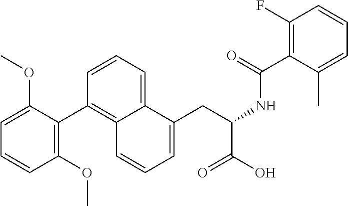 [SMILES: COc1cccc(OC)c1-c1cccc2c(C[C@H](NC(=O)c3c(C)cccc3F)C(=O)O)cccc12] |
| | m/z = 488.2 |
| 527 | 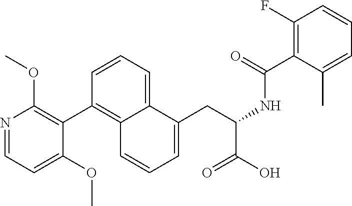 [SMILES: COc1ccnc(OC)c1-c1cccc2c(C[C@H](NC(=O)c3c(C)cccc3F)C(=O)O)cccc12] |
| | m/z = 489.2 |
| 528* | 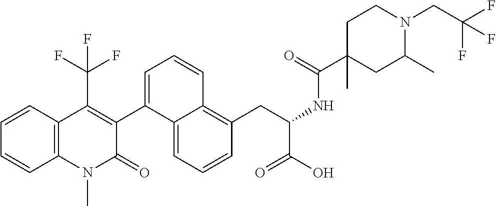 [SMILES: CC1CC(C)(C(=O)N[C@@H](Cc2cccc3c(-c4c(C(F)(F)F)c5ccccc5n(C)c4=O)cccc23)C(=O)O)CCN1CC(F)(F)F] |
| | m/z = 662.2 |
| 529* | 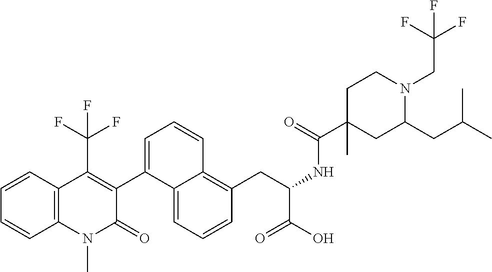 [SMILES: CC(C)CC1CC(C)(C(=O)N[C@@H](Cc2cccc3c(-c4c(C(F)(F)F)c5ccccc5n(C)c4=O)cccc23)C(=O)O)CCN1CC(F)(F)F] |
| | m/z = 704.5 |
| 530 | 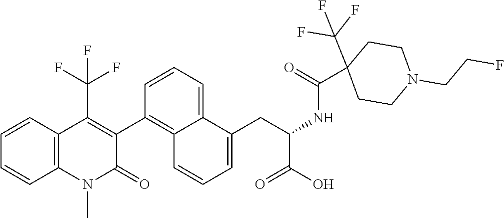 [SMILES: Cn1c(=O)c(-c2cccc3c(C[C@H](NC(=O)C4(C(F)(F)F)CCN(CCF)CC4)C(=O)O)cccc23)c(C(F)(F)F)c2ccccc21] |
| | m/z = 666.4 |
| 531 | 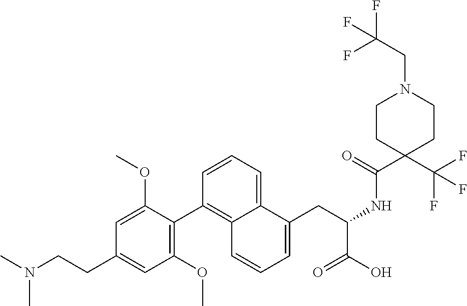 [SMILES: COc1cc(CCN(C)C)cc(OC)c1-c1cccc2c(C[C@H](NC(=O)C3(C(F)(F)F)CCN(CC(F)(F)F)CC3)C(=O)O)cccc12] |
| | m/z = 684.4 |
| 532* | 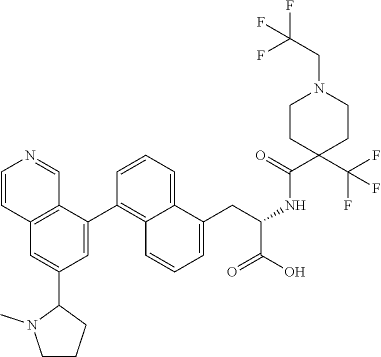 [SMILES: CN1CCCC1c1cc(-c2cccc3c(C[C@H](NC(=O)C4(C(F)(F)F)CCN(CC(F)(F)F)CC4)C(=O)O)cccc23)c2cnccc2c1] |
| | m/z = 687.3 |
| 533 | 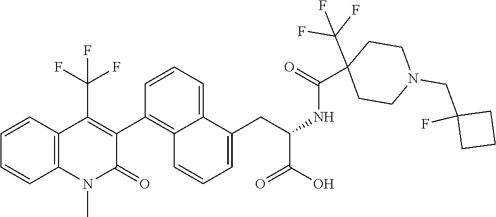 [SMILES: Cn1c(=O)c(-c2cccc3c(C[C@H](NC(=O)C4(C(F)(F)F)CCN(CC5(F)CCC5)CC4)C(=O)O)cccc23)c(C(F)(F)F)c2ccccc21] |
| | m/z = 706.2 |
| 534 | 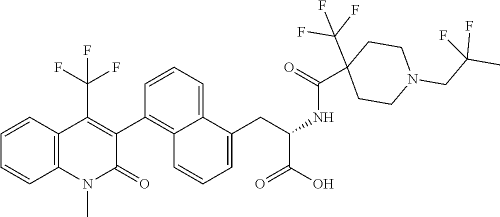 [SMILES: Cn1c(=O)c(-c2cccc3c(C[C@H](NC(=O)C4(C(F)(F)F)CCN(CC(C)(F)F)CC4)C(=O)O)cccc23)c(C(F)(F)F)c2ccccc21] |
| | m/z = 698.2 |
| 535 | 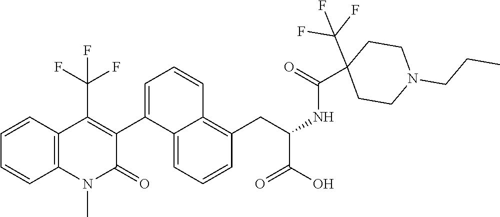 [SMILES: CCCN1CCC(C(=O)N[C@@H](Cc2cccc3c(-c4c(C(F)(F)F)c5ccccc5n(C)c4=O)cccc23)C(=O)O)(C(F)(F)F)CC1] |
| | m/z = 662.1 |
| 536 | 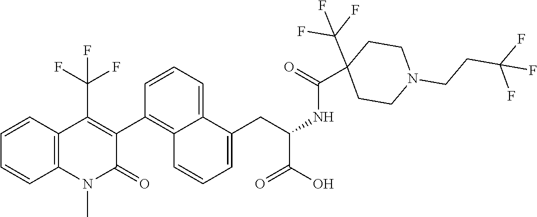 [SMILES: Cn1c(=O)c(-c2cccc3c(C[C@H](NC(=O)C4(C(F)(F)F)CCN(CCC(F)(F)F)CC4)C(=O)O)cccc23)c(C(F)(F)F)c2ccccc21] |
| | m/z = 716.2 |
| 537 | 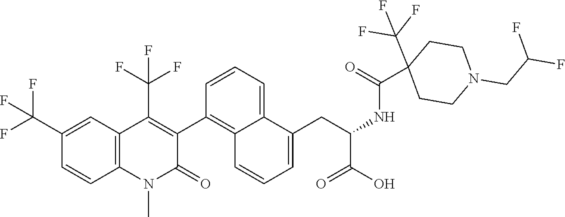 [SMILES: Cn1c(=O)c(-c2cccc3c(C[C@H](NC(=O)C4(C(F)(F)F)CCN(CC(F)F)CC4)C(=O)O)cccc23)c(C(F)(F)F)c2cc(C(F)(F)F)ccc21] |
| | m/z = 752.2 |
| 538 | 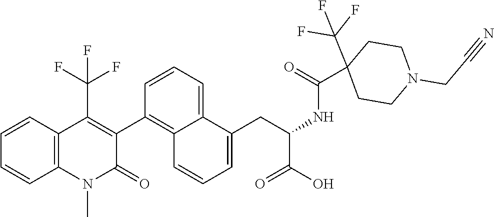 [SMILES: Cn1c(=O)c(-c2cccc3c(C[C@H](NC(=O)C4(C(F)(F)F)CCN(CC#N)CC4)C(=O)O)cccc23)c(C(F)(F)F)c2ccccc21] |
| | m/z = 659.1 |
| 539 | 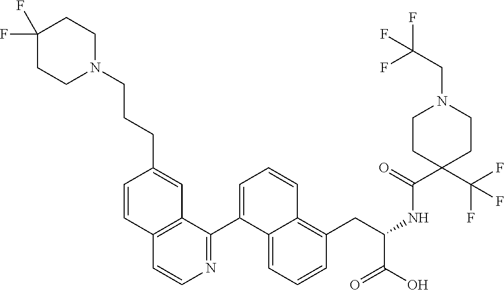 [SMILES: O=C(O)[C@H](Cc1cccc2c(-c3nccc4ccc(CCCN5CCC(F)(F)CC5)cc34)cccc12)NC(=O)C1(C(F)(F)F)CCN(CC(F)(F)F)CC1] |
| | m/z = 765.5 |
| 540 | 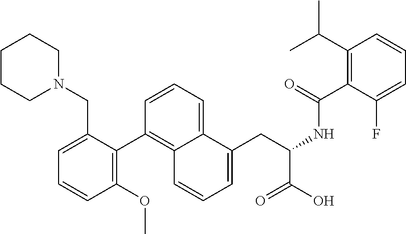 [SMILES: COc1cccc(CN2CCCCC2)c1-c1cccc2c(C[C@H](NC(=O)c3c(F)cccc3C(C)C)C(=O)O)cccc12] |
| | m/z = 583.3 |
| 541 | 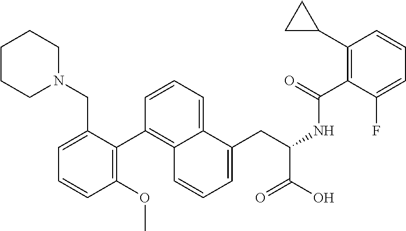 [SMILES: COc1cccc(CN2CCCCC2)c1-c1cccc2c(C[C@H](NC(=O)c3c(F)cccc3C3CC3)C(=O)O)cccc12] |
| | m/z = 581.2 |
| 542 | 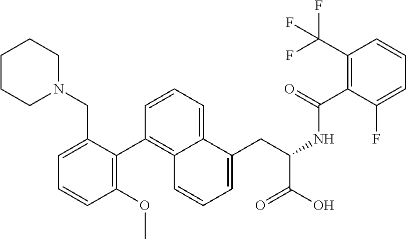 [SMILES: COc1cccc(CN2CCCCC2)c1-c1cccc2c(C[C@H](NC(=O)c3c(F)cccc3C(F)(F)F)C(=O)O)cccc12] |
| | m/z = 609.3 |
| 543 | 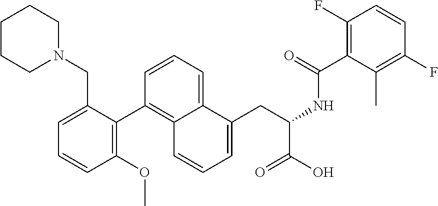 [SMILES: COc1cccc(CN2CCCCC2)c1-c1cccc2c(C[C@H](NC(=O)c3c(F)ccc(F)c3C)C(=O)O)cccc12] |
| | m/z = 573.3 |
| 544 | 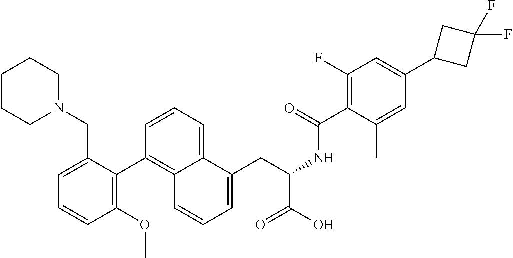 [SMILES: COc1cccc(CN2CCCCC2)c1-c1cccc2c(C[C@H](NC(=O)c3c(C)cc(C4CC(F)(F)C4)cc3F)C(=O)O)cccc12] |
| | m/z = 645.2 |
| 545 | 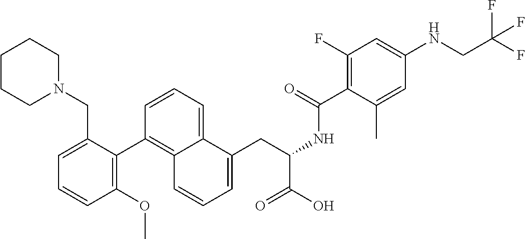 [SMILES: COc1cccc(CN2CCCCC2)c1-c1cccc2c(C[C@H](NC(=O)c3c(C)cc(NCC(F)(F)F)cc3F)C(=O)O)cccc12] |
| | m/z = 652.4 |
| 546 | 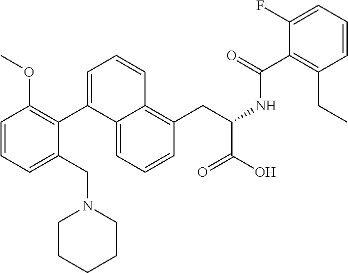 [SMILES: CCc1cccc(F)c1C(=O)N[C@@H](Cc1cccc2c(-c3c(CN4CCCCC4)cccc3OC)cccc12)C(=O)O] |
| | m/z = 569.3 |
| 547 | 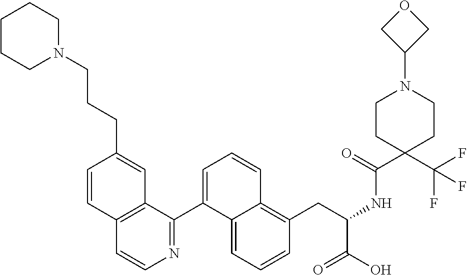 [SMILES: O=C(O)[C@H](Cc1cccc2c(-c3nccc4ccc(CCCN5CCCCC5)cc34)cccc12)NC(=O)C1(C(F)(F)F)CCN(C2COC2)CC1] |
| | m/z = 703.2 |
| 548* | 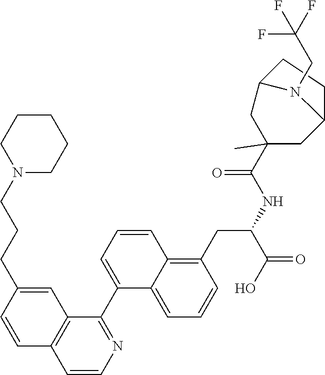 [SMILES: CC1(C(=O)N[C@@H](Cc2cccc3c(-c4nccc5ccc(CCCN6CCCCC6)cc45)cccc23)C(=O)O)CC2CCC(C1)N2CC(F)(F)F] |
| | m/z = 701.3 |
| 549 | 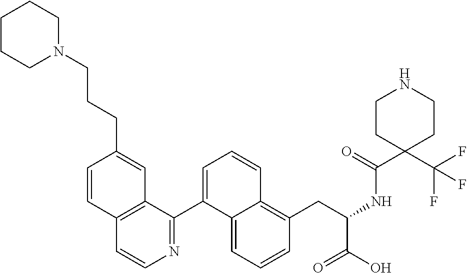 [SMILES: O=C(O)[C@H](Cc1cccc2c(-c3nccc4ccc(CCCN5CCCCC5)cc34)cccc12)NC(=O)C1(C(F)(F)F)CCNCC1] |
| | m/z = 647.4 |
| 550 | 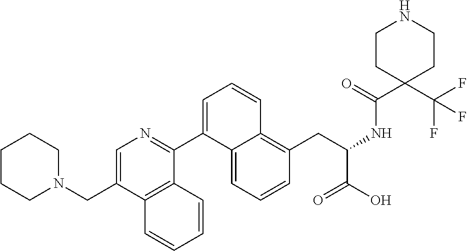 [SMILES: O=C(O)[C@H](Cc1cccc2c(-c3ncc(CN4CCCCC4)c4ccccc34)cccc12)NC(=O)C1(C(F)(F)F)CCNCC1] |
| | m/z = 619.3 |
| 567 | 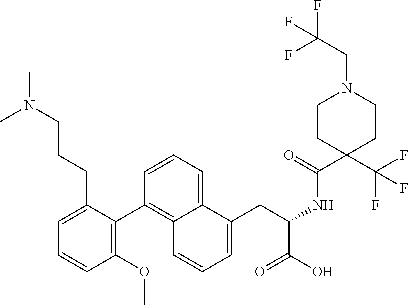 [SMILES: COc1cccc(CCCN(C)C)c1-c1cccc2c(C[C@H](NC(=O)C3(C(F)(F)F)CCN(CC(F)(F)F)CC3)C(=O)O)cccc12] |
| | m/z = 668.2 |

*Denotes a racemic compound;
**Denotes a single isomer with unknown absolute stereochemistry

### Example 30: General Scheme 1B

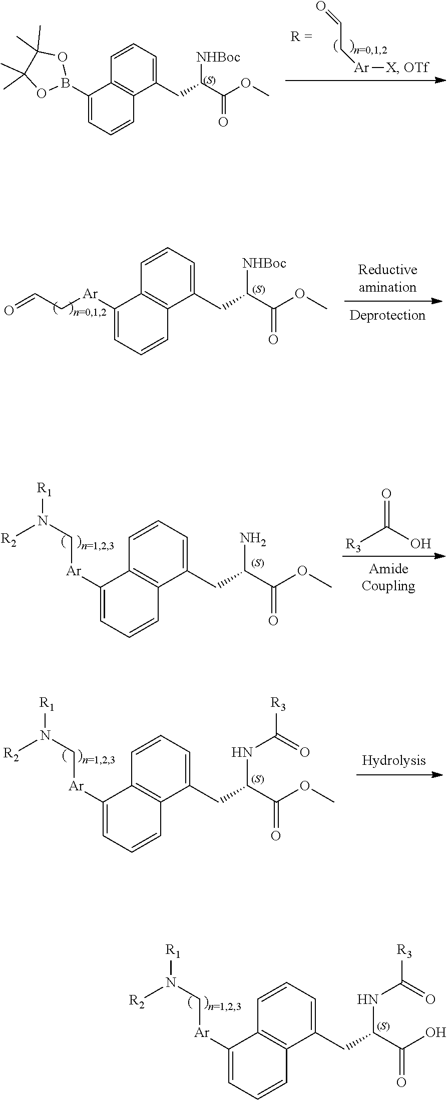

The proceeding compounds were synthesized according to the procedures described in General Scheme 1B using Int1 as an intermediate.

### Synthesis of Compound 551

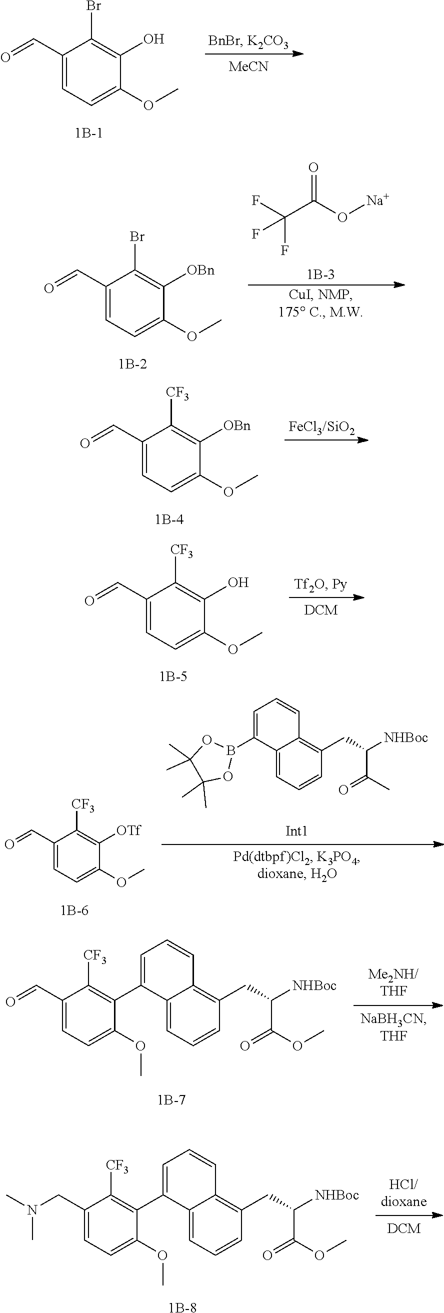

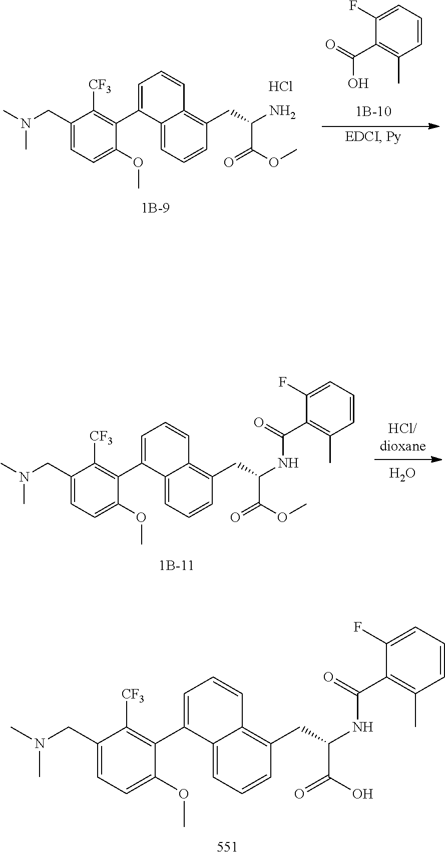

Step 1: To a solution of compound 1B-1 (4.00 g, 17.3 mmol, 1.00 eq) in MeCN (40.0 mL) was added K₂CO₃ (4.79 g, 34.6 mmol, 2.00 eq) and BnBr (4.44 g, 26.0 mmol, 3.08 mL, 1.50 eq), the mixture was stirred at 25° C. for 2 hrs. The reaction mixture was diluted with H₂O (50.0 mL) and extracted with ethyl acetate (50.0 mL*3). The combined organic layers were washed with brine (50.0 mL*3), dried over Na₂SO₄, filtered and concentrated under reduced pressure to give a residue. The residue was purified by column chromatography (SiO₂, Petroleum ether/Ethyl acetate=100/1 to 10/1). Compound 1B-2 (3.90 g, 12.1 mmol, 70.1% yield) was obtained as yellow solid. ¹H NMR: (400 MHz, CDCl₃) δ 10.28 (d, J=0.8 Hz, 1H), 7.77 (d, J=8.8 Hz, 1H), 7.59-7.53 (m, 2H), 7.44-7.33 (m, 3H), 6.98 (d, J=8.4 Hz, 1H), 5.06 (s, 2H), 3.96 (s, 3H).

Step 2: Compound 1B-2 (0.500 g, 1.56 mmol, 1.00 eq), compound 1B-3 (847 mg, 6.23 mmol, 4.00 eq) and CuI (593 mg, 3.11 mmol, 2.00 eq) were taken up into a microwave tube in NMP (10.0 mL). The sealed tube was heated at 175° C. for 4 hrs under microwave. The residue was diluted with H₂O (50.0 mL) and extracted with ethyl acetate (30.0 mL*3). The combined organic layers were washed with brine (30.0 mL*3), dried over Na₂SO₄, filtered and concentrated under reduced pressure to give a residue. The residue was purified by column chromatography (SiO₂, Petroleum ether/Ethyl acetate=100/1 to 1/1). Compound 1B-4 (1.40 g, 4.51 mmol, 48.3% yield) was obtained as yellow solid. Compound 1B-4 (1.80 g, 3.13 mmol, 33.5% yield, 54% purity) was obtained as yellow solid. LC-MS: (M+H)⁺: 310.9. ¹H NMR: (400 MHz, DMSO-d₆) δ 10.21-10.15 (m, 1H), 7.77 (d, J=8.8 Hz, 1H), 7.55 (d, J=8.8 Hz, 1H), 7.48-7.33 (m, 5H), 5.05 (s, 2H), 4.01 (s, 3H).

Step 3: To a solution of FeCl₃ (812 mg, 5.01 mmol, 290 μL, 1.11 eq) in THF (126 mL) and MeOH (14.0 mL) was added SiO₂ (17.6 g, 294 mmol, 65.1 eq). The mixture was concentrated under reduced pressure (water pump) on the rotary evaporator and remained spinning for 30 min at 30° C. Then the mixture was transferred to a vacuum (oil pump) at 30° C. for 1 hr to get a yellow solid (18.5 g, 4.40% FeCl₃/SiO₂). To a solution of compound 1B-4 (1.40 g, 4.51 mmol, 1.00 eq) in DCM (20.0 mL) was added FeCl₃/SiO₂ (the same as above, 18.5 g) at 25° C. An immediate deep red color formed on the surface of the reagent. The mixture was removed under reduced pressure on the rotary evaporator and remained spinning for 2 hrs at 45° C. The reaction mixture was quenched by H₂O (80.0 mL) at 25° C. and diluted with ethyl acetate (50.0 mL). The reaction mixture was filtered and the filter extracted with ethyl acetate (50.0 mL*3). The combined organic layers were washed with brine (50.0 mL*3), dried over Na₂SO₄, filtered and concentrated under reduced pressure to give a residue. The residue was purified by column chromatography (SiO₂, Petroleum ether/Ethyl acetate-100/1 to 1/1). Compound 1B-5 (0.600 g, 2.73 mmol, 60.4% yield) was obtained as yellow solid. ¹H NMR: (400 MHz, CDCl₃) δ 10.32-10.28 (m, 1H), 7.66 (d, J=8.0 Hz, 1H), 7.08 (d, J=8.8 Hz, 1H), 6.44-6.32 (m, 1H), 4.04 (s, 3H).

Step 4: To a solution of compound 1B-5 (0.600 g, 2.73 mmol, 1.00 eq) in DCM (10.0 mL) was added Py (2.16 g, 27.3 mmol, 2.20 mL, 10.0 eq) and Tf₂O (2.31 g, 8.18 mmol, 1.35 mL, 3.00 eq) at 0° C., then the mixture was stirred at 25° C. for 1 hr. The residue was diluted with H₂O (20.0 mL) and extracted with ethyl acetate (20.0 mL*3). The combined organic layers were washed with brine (20.0 mL*2), dried over Na₂SO₄, filtered, and concentrated under reduced pressure to give a residue. The residue was purified by Prep-TLC (SiO₂, Petroleum ether/Ethyl acetate=3/1).

Compound 1B-6 (0.650 g, 1.85 mmol, 67.7% yield) was obtained as yellow oil. LC-MS: (M+H)⁺: 352.8. ¹H NMR: (400 MHz, CDCl₃) δ 10.29-10.24 (m, 1H), 8.16 (d, J=8.8 Hz, 1H), 7.37 (d, J=8.8 Hz, 1H), 4.06 (s, 3H)

Step 5: To a solution of compound 1B-6 (0.150 g, 426 μmol, 1.00 eq) and compound Int1 (291 mg, 639 μmol, 1.50 eq) in dioxane (5.00 mL) and H₂O (1.00 mL) was added K₃PO₄ (181 mg, 852 μmol, 2.00 eq) and Pd(dtbpf)Cl₂ (27.8 mg, 42.6 μmol, 0.100 eq) at 25° C., then the mixture was stirred at 40° C. for 2 hrs. The reaction mixture was diluted with H₂O (40.0 mL) and extracted with ethyl acetate (30.0 mL*3). The combined organic layers were washed with brine (30.0 mL*3), dried over Na₂SO₄, filtered, and concentrated under reduced pressure to give a residue. The residue was purified by Prep-TLC (SiO₂, Petroleum ether/Ethyl acetate=1/1). Compound 1B-7 (0.820 g, 739 μmol, 43.4% yield, 47.9% purity) was obtained as yellow oil. LC-MS: (M-99): 432.1

Step 6: To a solution of compound 1B-7 (300 mg, 270 μmol, 1.00 eq) in THF (6.00 mL) was added Me₂NH (2.00 M, 1.00 mL, 7.40 eq), the mixture was stirred at 25° C. for 12 hrs. Then NaBH₃CN (20.4 mg, 324 μmol, 1.20 eq) was added and the mixture was stirred at 25° C. for 1 hr. The residue was diluted with H₂O (20.0 mL) and extracted with ethyl acetate (20.0 mL*3). The combined organic layers were washed with brine (20.0 mL*3), dried over Na₂SO₄, filtered, and concentrated under reduced pressure to give a residue. The residue was purified by Prep-TLC (SiO₂, DCM/MeOH-10/1). Compound 1B-8 (0.110 g, 196 μmol, 72.6% yield) was obtained as yellow oil. LC-MS: (M+H)⁺: 561.6

Step 7: To a solution of compound 1B-8 (110 mg, 196 μmol, 1.00 eq) in DCM (2.00 mL) was added HCl/dioxane (4.00 M, 0.500 mL, 10.2 eq), the mixture was stirred at 25° C. for 1 hr. The reaction mixture was concentrated under reduced pressure to give a residue. Compound 1B-9 (90.0 mg, 181 μmol, 92.3% yield, HCl) was obtained as yellow solid. LC-MS: (M+H)⁺: 461.3

Step 8: To a solution of compound 1B-9 (70.0 mg, 141 μmol, 1.00 eq, HCl) and compound 1B-10 (26.1 mg, 169 μmol, 1.20 eq) in Py (2.00 mL) was added EDCI (54.0 mg, 282 μmol, 2.00 eq), then the mixture was stirred at 25° C. for 1 hr. The residue was diluted with sat. aq. NaHCO₃ (20.0 mL) and extracted with ethyl acetate (20.0 mL*3). The combined organic layers were washed with brine (20.0 mL*3), dried over Na₂SO₄, filtered, and concentrated under reduced pressure to give a residue. Compound 1B-11 (70.0 mg, 117 μmol, 83.3% yield) was obtained as yellow solid. LC-MS: (M+H)⁺: 597.5

Step 9: To a solution of compound 1B-11 (60.0 mg, 101 μmol, 1.00 eq) in H₂O (1.00 mL) was added HCl/dioxane (4.00 M, 6.00 mL, 239 eq), the mixture was stirred at 60° C. for 1 hr. The residue was purified by Prep-HPLC (column: Phenomenex luna C18 150*25 mm*10 um; mobile phase: [water (FA)-ACN]; B %: 13%-43%, 15 min). Compound 551 (37.83 mg, 63.5 μmol, 63.2% yield, 97.8% purity) was obtained as white solid. LC-MS: (M+H)⁺: 583.2. ¹H NMR: (400 MHz, DMSO-d₆) $8.99 (dd, J₁=20.0 Hz, J₂=8.0 Hz, 1H), 8.15 (dd, J₁=8.4 Hz, J₂=2.8 Hz, 1H), 7.80 (d, J=8.4 Hz, 1H), 7.65-7.57 (m, 1H), 7.48-7.38 (m, 2H), 7.34-7.20 (m, 3H), 7.10 (d, J=8.4 Hz, 1H), 7.05-6.96 (m, 2H), 4.89-4.78 (m, 1H), 3.75 (br dd, J₁=14.4 Hz, J₂=3.2 Hz, 1H), 3.56 (d, J=6.4 Hz, 5H), 3.54-3.50 (m, 1H), 2.23 (s, 6H), 1.96 (d, J=43.2 Hz, 3H).

### Synthesis of Compound 552

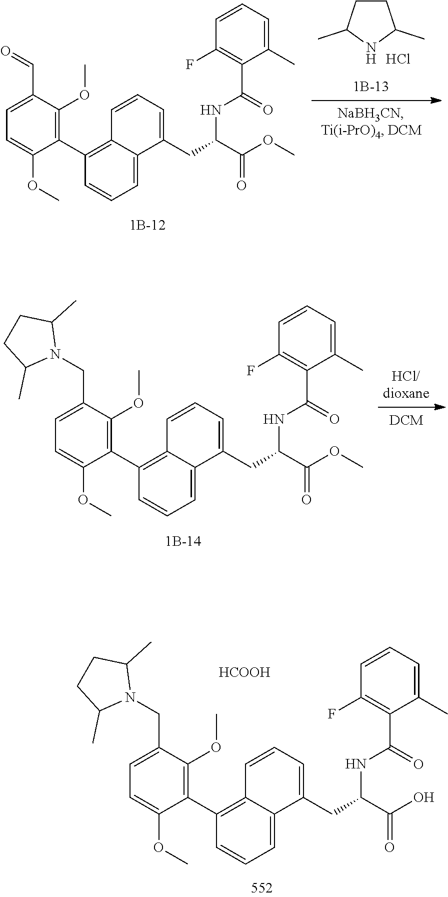

Step 1: The mixture of compound 1B-12 (100 mg, 177 μmol, 1.00 eq), compound 1B-13 (35.0 mg, 258 μmol, 43.3μ, 1.46 eq, HCl) and Ti(i-PrO)₄ (50.2 mg, 177 μmol, 52.1μ, 1.00 eq) in DCM (1.00 mL) was stirred at 25° C. over 11 hrs. After addition, then NaBH₃CN (33.3 mg, 530 μmol, 3.00 eq) was stirred at for 1 hr. The reaction mixture was diluted with water 20.0 mL and extracted with ethyl acetate (25.0 mL*3). The combined organic layers were washed with NaCl (10.0 mL*3), dried over Na₂SO₄, filtered, and concentrated under reduced pressure to give a residue. The residue was purified by prep-TLC (Plate 1, SiO₂, DCM:MeOH=7:1). Compound 1B-14 (70.0 mg, 114. μmol, 64.7% yield) was obtained as a white solid. LC-MS: (M+H)⁺: 613.3. ¹H NMR: (400 MHz, CDCl₃). δ 8.23 (dd, J₁=8.4 Hz, J₂=2.4 Hz, 1H), 7.66-7.62 (m, 1H), 7.56-7.52 (m, 1H), 7.43-7.41 (m, 2H), 7.35-7.28 (m, 2H), 7.25-7.21 (m, 1H), 7.01-6.84 (m, 3H), 6.42-6.37 (m, 1H), 5.31-5.26 (m, 1H), 4.04-3.99 (m, 2H), 3.82-3.76 (m, 1H), 3.74-3.71 (m, 3H), 3.66-3.60 (m, 5H), 3.12-3.10 (m, 4H), 2.33-2.24 (m, 3H), 2.07-2.01 (m, 2H), 1.30-1.24 (m, 8H).

Step 2: To a solution of compound 1B-14 (65.0 mg, 106 μmol, 1.00 eq) in H₂O (1.00 mL) was added HCl/dioxane (4 M, 2.00 mL, 75.4 eq). The mixture was stirred at 60° C. for 1 hr. The reaction mixture was concentrated under reduced pressure to give a residue. The residue was purified by prep-HPLC (column: Phenomenex luna C 18 150*25 mm*10 um; mobile phase: [water (FA)-ACN]; gradient: 18%-48% B over 10 min). Compound 552 (41.65 mg, 64.6 μmol, 60.9% yield, 100% purity, FA) was obtained as a white solid. LC-MS: (M+H)⁺: 599.4. ¹H NMR: (400 MHz, DMSO-d₆) δ 9.00-8.90 (m, 1H), 8.20-8.16 (m, 2H), 7.67-7.63 (m, 1H), 7.54-7.49 (m, 1H), 7.44-7.42 (m, 1H), 7.38-7.74 (m, 1H), 7.32-7.22 (br s, 3H), 7.01 (brt, J=8.0 Hz, 2H), 6.94-6.91 (m, 1H), 4.84-4.86 (m, 1H), 3.81-3.71 (m, 3H), 3.57 (d, J=9.2 Hz, 3H), 3.20-3.07 (m, 4H), 2.81-2.69 (m, 1H), 2.04-1.96 (m, 4H), 1.88-1.79 (m, 2H), 1.40-1.35 (m, 2H), 1.04-1.00 (m, 6H).

### Synthesis of Compound 553

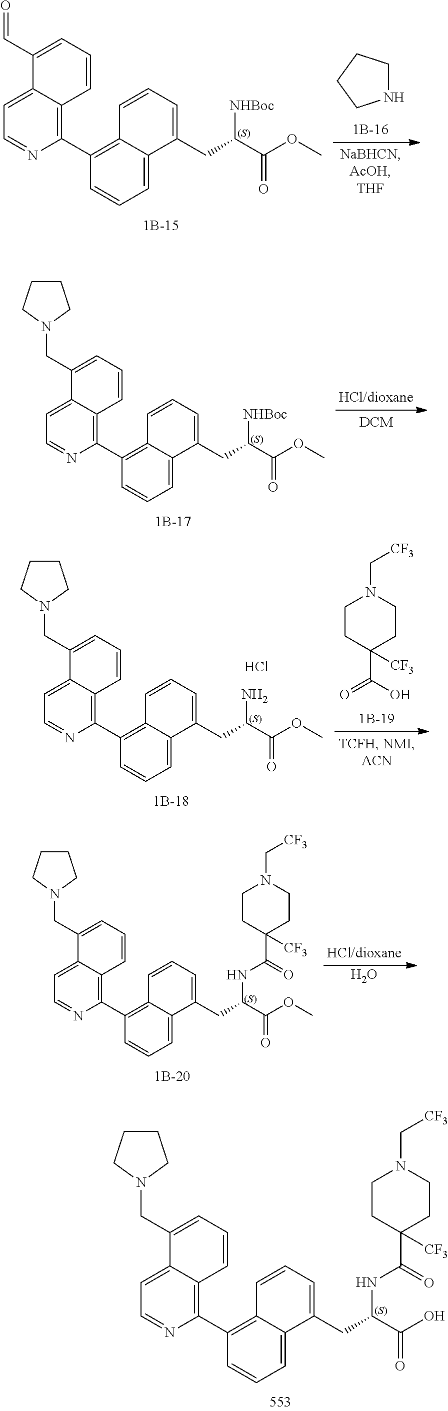

Step 1: To a solution of compound 1B-15 (120 mg, 247 μmol, 1.00 eq), compound 1B-16 (88.0 mg, 1.24 mmol, 103 μL, 5.00 eq), AcOH (104 mg, 1.75 mmol, 0.100 mL, 7.05 eq) in THF (0.500 mL) was stirred at 25° C. for 10 hrs, then NaBH₃CN (23.3 mg, 371 μmol, 1.50 eq) was added. The mixture was stirred at 25° C. for 2 hrs. The reaction mixture was quenched by addition sat. aq. NaHCO₃20.0 mL at 25° C., and then diluted with H₂O 30.0 mL and extracted with ethyl acetate 45.0 mL (15.0 mL*3). The combined organic layers were dried over Na 2SO₄, filtered, and concentrated under reduced pressure to give a residue. The residue was purified by Prep-TLC (SiO₂, Petroleum ether:Ethyl acetate=1:1). Compound 1B-17 (100 mg, 185 μmol, 74.8% yield) was obtained as a yellow solid. LC-MS: (M+H)⁺: 540.3. ¹H NMR: (400 MHz, DMSO-d₆) δ 8.68 (d, J=5.6 Hz, 1H), 8.24-8.21 (m, 2H), 7.79-7.67 (m, 2H), 7.56 (d, J=7.2 Hz, 1H), 7.43-7.41 (m, 5H), 7.10 (dd, J₁=8.4 Hz, J₂=3.6 Hz, 1H), 4.40-4.39 (m, 1H), 3.94-3.86 (m, 2H), 3.68-3.64 (m, 4H), 3.32-3.29 (m, 1H), 2.54-2.50 (m, 4H), 1.76 (s, 4H), 1.30 (s, 9H).

Step 2: To a solution of compound 1B-17 (100 mg, 185 μmol, 1.00 eq) in DCM (0.500 mL) was added HCl/dioxane (4.00 M, 0.500 mL, 10.7 eq). The mixture was stirred at 25° C. for 0.5 hr. The combined organic layers were concentrated under reduced pressure to give a residue. The reaction mixture was quenched by addition sat. aq. NaHCO₃ 100 mL at 25° C., and then diluted with H₂O 50.0 mL and extracted with DCM 45.0 mL (15.0 mL*3). Compound 1B-18 (75.0 mg, crude) was obtained as a yellow solid. LC-MS: (M+H)⁺: 440.3.

Step 3: To a solution of compound 1B-18 (65.0 mg, 147 μmol, 1.00 eq) in ACN (2.00 mL) was compound 1B-19 (49.5 mg, 177 μmol, 1.20 eq), TCFH (103 mg, 369 μmol, 2.50 eq) and NMI (48.5 mg, 591 μmol, 47.1 μL, 4.00 eq). The mixture was stirred at 25° C. for 2 hrs. The reaction mixture was concentrated under reduced pressure to give a residue, the residue was purified by Prep-TLC. Compound 1B-20 (60.0 mg, 85.6 μmol, 57.9% yield) was obtained as a yellow oil. LC-MS: (M+H)⁺: 701.3.

Step 4: To a solution of compound 1B-20 (60.0 mg, 85.6 μmol, 1.00 eq) in HCl/dioxane (4.00 M, 21.4 μL, 1.00 eq) was added H₂O (0.500 mL). The mixture was stirred at 60° C. for 2 hrs. The reaction mixture was concentrated under reduced pressure to give a residue. The residue was purified by Prep-HPLC (column: Xtimate C18 150*40 mm*10 um; mobile phase: [water (HCl)-ACN]; B %: 12%-42%, 10 min) and futher purified by Prep-HPLC (column: Waters xbridge 150*25 mm 10 um; mobile phase: [water (NH₄HCO₃)-ACN]; B %: 30%-60%, 10 min). Compound 553 23.07 mg, 33.6 μmol, 39.2% yield, 100% purity) was obtained as a white solid. LC-MS: (M+H)⁻: 687.2. ¹H NMR: (400 MHz, DMSO-d₆) δ8.65 (br dd, J₁=6.0 Hz, J₂=1.6 Hz, 1H), 8.44-8.28 (m, 2H), 8.22 (d, J=6.0 Hz, 1H), 7.82-7.65 (m, 2H), 7.55 (d, J=7.2 Hz, 1H), 7.45-7.36 (m, 2H), 7.33-7.17 (m, 2H), 7.05 (d, J=8.4 Hz, 1H), 4.87-4.81 (m, 1H), 4.18-3.97 (m, 2H), 3.93-3.74 (m, 1H), 3.52-3.48 (m, 1H), 2.99-2.63 (m, 6H), 2.24-1.52 (m, 12H).

The compounds set forth in Table 15, were prepared using the synthetic procedures of Example 30, or analogous procedures as provided herein.

TABLE 15
Structure and Spectroscopic data for Compounds 554 to 694, 823, and 850

| No. | Structure |
|---|---|
| 554 | 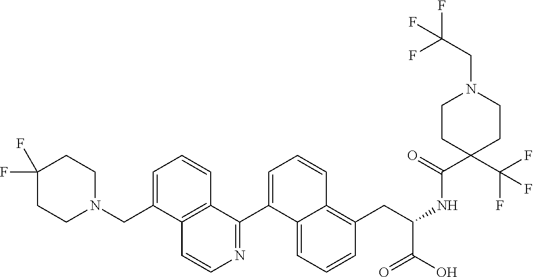 [SMILES: O=C(O)[C@H](Cc1cccc2c(-c3nccc4c(CN5CCC(F)(F)CC5)cccc34)cccc12)NC(=O)C1(C(F)(F)F)CCN(CC(F)(F)F)CC1] |
| | m/z = 737.2 |
| 555 | 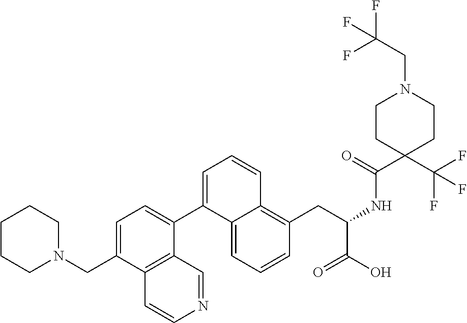 [SMILES: O=C(O)[C@H](Cc1cccc2c(-c3ccc(CN4CCCCC4)c4ccncc34)cccc12)NC(=O)C1(C(F)(F)F)CCN(CC(F)(F)F)CC1] |
| | m/z = 701.2 |
| 556 | 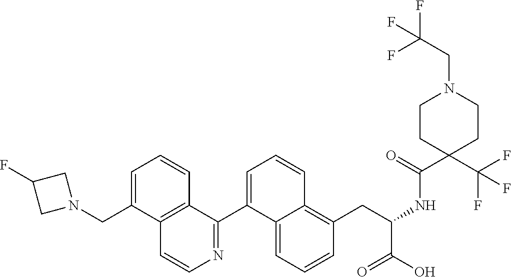 [SMILES: O=C(O)[C@H](Cc1cccc2c(-c3nccc4c(CN5CC(F)C5)cccc34)cccc12)NC(=O)C1(C(F)(F)F)CCN(CC(F)(F)F)CC1] |
| | m/z = 691.2 |
| 557 | 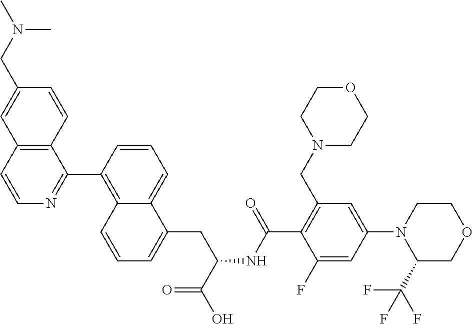 [SMILES: CN(C)Cc1ccc2c(-c3cccc4c(C[C@H](NC(=O)c5c(F)cc(N6CCOC[C@@H]6C(F)(F)F)cc5CN5CCOCC5)C(=O)O)cccc34)nccc2c1] |
| | m/z = 774.3 |
| 558 | 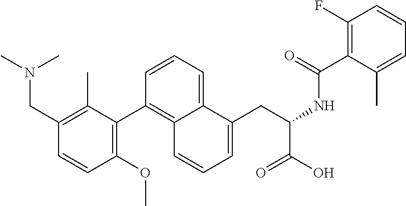 [SMILES: COc1ccc(CN(C)C)c(C)c1-c1cccc2c(C[C@H](NC(=O)c3c(C)cccc3F)C(=O)O)cccc12] |
| | m/z = 529.2 |
| 559 | 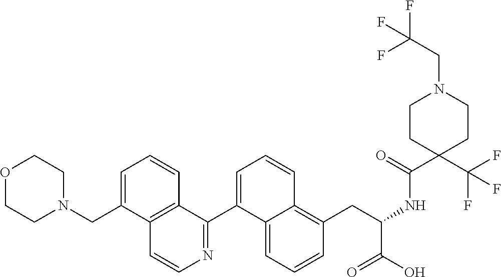 [SMILES: O=C(O)[C@H](Cc1cccc2c(-c3nccc4c(CN5CCOCC5)cccc34)cccc12)NC(=O)C1(C(F)(F)F)CCN(CC(F)(F)F)CC1] |
| | m/z = 703.2 |
| 560 | 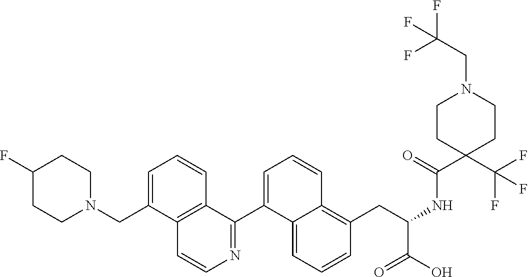 [SMILES: O=C(O)[C@H](Cc1cccc2c(-c3nccc4c(CN5CCC(F)CC5)cccc34)cccc12)NC(=O)C1(C(F)(F)F)CCN(CC(F)(F)F)CC1] |
| | m/z = 719.3 |
| 561 | 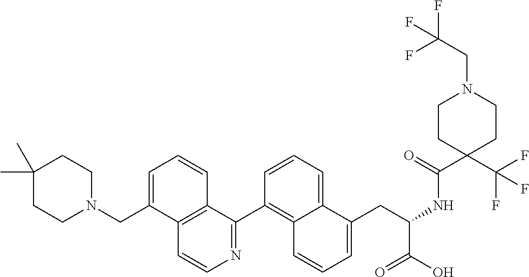 [SMILES: CC1(C)CCN(Cc2cccc3c(-c4cccc5c(C[C@H](NC(=O)C6(C(F)(F)F)CCN(CC(F)(F)F)CC6)C(=O)O)cccc45)nccc23)CC1] |
| | m/z = 729.5 |
| 562 | 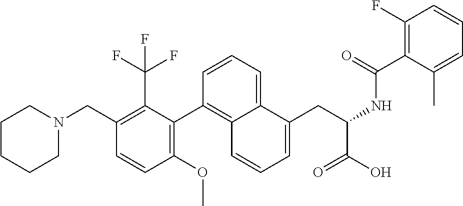 [SMILES: COc1ccc(CN2CCCCC2)c(C(F)(F)F)c1-c1cccc2c(C[C@H](NC(=O)c3c(C)cccc3F)C(=O)O)cccc12] |
| | m/z = 623.3 |
| 563 | 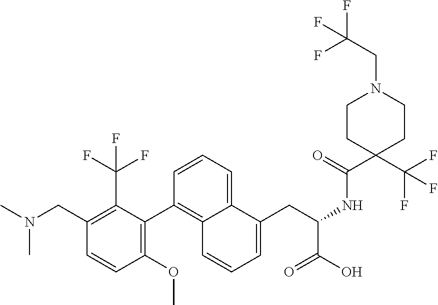 [SMILES: COc1ccc(CN(C)C)c(C(F)(F)F)c1-c1cccc2c(C[C@H](NC(=O)C3(C(F)(F)F)CCN(CC(F)(F)F)CC3)C(=O)O)cccc12] |
| | m/z = 708.2 |
| 564 | 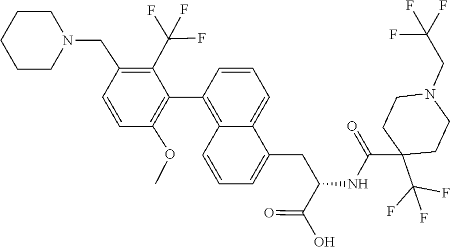 [SMILES: COc1ccc(CN2CCCCC2)c(C(F)(F)F)c1-c1cccc2c(C[C@H](NC(=O)C3(C(F)(F)F)CCN(CC(F)(F)F)CC3)C(=O)O)cccc12] |
| | m/z = 748.2 |
| 565 | 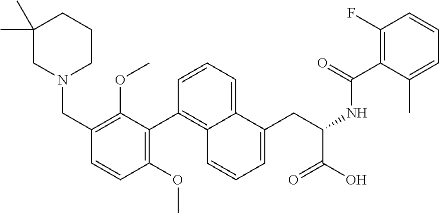 [SMILES: COc1ccc(CN2CCCC(C)(C)C2)c(OC)c1-c1cccc2c(C[C@H](NC(=O)c3c(C)cccc3F)C(=O)O)cccc12] |
| | m/z = 613.3 |
| 566 | 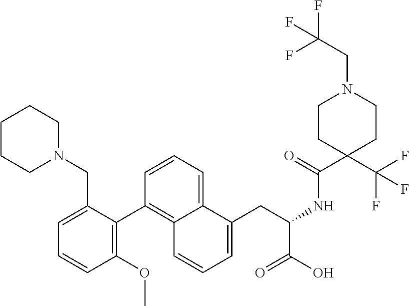 [SMILES: COc1cccc(CN2CCCCC2)c1-c1cccc2c(C[C@H](NC(=O)C3(C(F)(F)F)CCN(CC(F)(F)F)CC3)C(=O)O)cccc12] |
| | m/z = 680.3 |
| 568 | 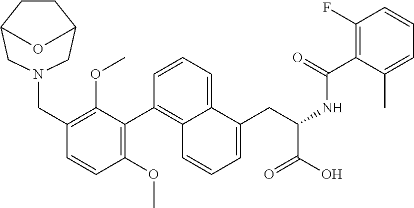 [SMILES: COc1ccc(CN2CC3CCC(C2)O3)c(OC)c1-c1cccc2c(C[C@H](NC(=O)c3c(C)cccc3F)C(=O)O)cccc12] |
| | m/z = 613.3 |
| 569 | 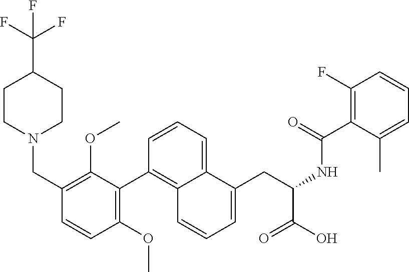 [SMILES: COc1ccc(CN2CCC(C(F)(F)F)CC2)c(OC)c1-c1cccc2c(C[C@H](NC(=O)c3c(C)cccc3F)C(=O)O)cccc12] |
| | m/z = 653.4 |
| 570 | 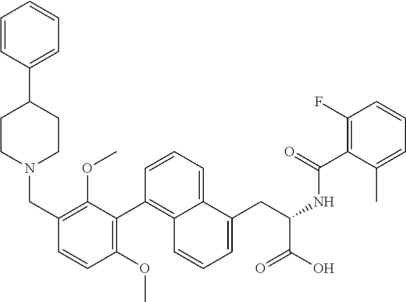 [SMILES: COc1ccc(CN2CCC(c3ccccc3)CC2)c(OC)c1-c1cccc2c(C[C@H](NC(=O)c3c(C)cccc3F)C(=O)O)cccc12] |
| | m/z = 661.5 |
| 571 | 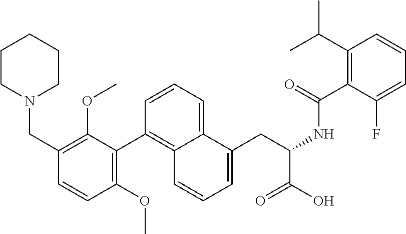 [SMILES: COc1ccc(CN2CCCCC2)c(OC)c1-c1cccc2c(C[C@H](NC(=O)c3c(F)cccc3C(C)C)C(=O)O)cccc12] |
| | m/z = 613.3 |
| 572* | 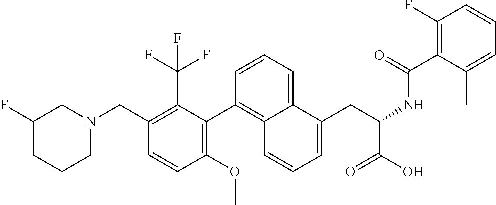 [SMILES: COc1ccc(CN2CCCC(F)C2)c(C(F)(F)F)c1-c1cccc2c(C[C@H](NC(=O)c3c(C)cccc3F)C(=O)O)cccc12] |
| | m/z = 641.1 |
| 573** | 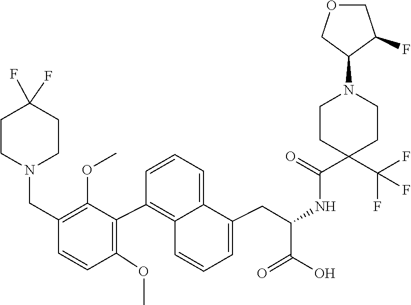 [SMILES: COc1ccc(CN2CCC(F)(F)CC2)c(OC)c1-c1cccc2c(C[C@H](NC(=O)C3(C(F)(F)F)CCN([C@H]4COC[C@H]4F)CC3)C(=O)O)cccc12] |
| | m/z = 752.4 |
| 574 | 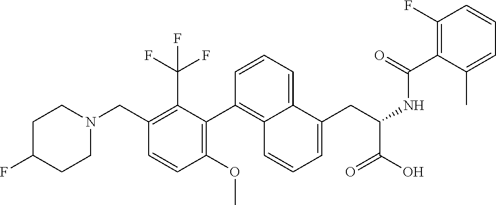 [SMILES: COc1ccc(CN2CCC(F)CC2)c(C(F)(F)F)c1-c1cccc2c(C[C@H](NC(=O)c3c(C)cccc3F)C(=O)O)cccc12] |
| | m/z = 641.1 |
| 575 | 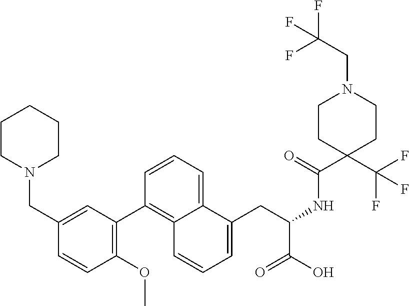 [SMILES: COc1ccc(CN2CCCCC2)cc1-c1cccc2c(C[C@H](NC(=O)C3(C(F)(F)F)CCN(CC(F)(F)F)CC3)C(=O)O)cccc12] |
| | m/z = 680.3 |
| 576 | 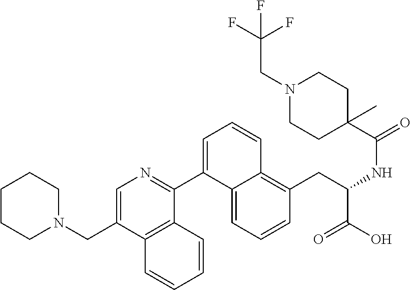 [SMILES: CC1(C(=O)N[C@@H](Cc2cccc3c(-c4ncc(CN5CCCCC5)c5ccccc45)cccc23)C(=O)O)CCN(CC(F)(F)F)CC1] |
| | m/z = 647.2 |
| 577 | 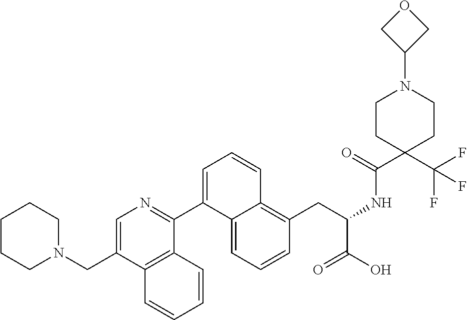 [SMILES: O=C(O)[C@H](Cc1cccc2c(-c3ncc(CN4CCCCC4)c4ccccc34)cccc12)NC(=O)C1(C(F)(F)F)CCN(C2COC2)CC1] |
| | m/z = 675.4 |
| 578 | 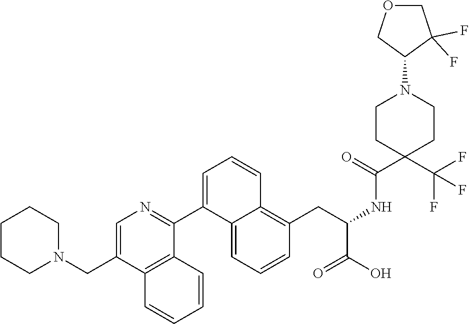 [SMILES: O=C(O)[C@H](Cc1cccc2c(-c3ncc(CN4CCCCC4)c4ccccc34)cccc12)NC(=O)C1(C(F)(F)F)CCN([C@@H]2COCC2(F)F)CC1] |
| | m/z = 725.4 |
| 579 | 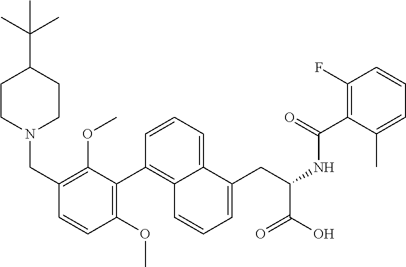 [SMILES: COc1ccc(CN2CCC(C(C)(C)C)CC2)c(OC)c1-c1cccc2c(C[C@H](NC(=O)c3c(C)cccc3F)C(=O)O)cccc12] |
| | m/z = 641.2 |
| 580 | 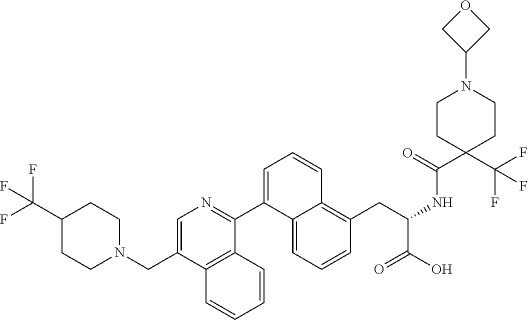 [SMILES: O=C(O)[C@H](Cc1cccc2c(-c3ncc(CN4CCC(C(F)(F)F)CC4)c4ccccc34)cccc12)NC(=O)C1(C(F)(F)F)CCN(C2COC2)CC1] |
| | m/z = 743.3 |
| 581** | 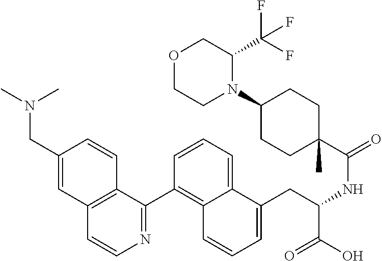 [SMILES: CN(C)Cc1ccc2c(-c3cccc4c(C[C@H](NC(=O)[C@]5(C)CC[C@@H](N6CCOC[C@@H]6C(F)(F)F)CC5)C(=O)O)cccc34)nccc2c1] |
| | m/z = 677.3 |
| 582** | 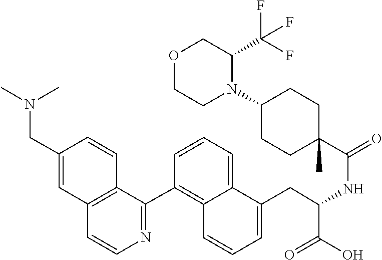 [SMILES: CN(C)Cc1ccc2c(-c3cccc4c(C[C@H](NC(=O)[C@]5(C)CC[C@H](N6CCOC[C@@H]6C(F)(F)F)CC5)C(=O)O)cccc34)nccc2c1] |
| | m/z = 677.3 |
| 583 | 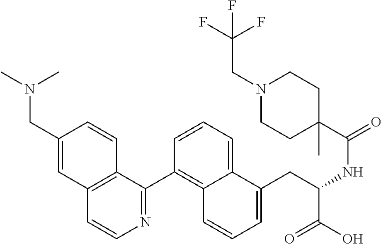 [SMILES: CN(C)Cc1ccc2c(-c3cccc4c(C[C@H](NC(=O)C5(C)CCN(CC(F)(F)F)CC5)C(=O)O)cccc34)nccc2c1] |
| | m/z = 607.3 |
| 584 | 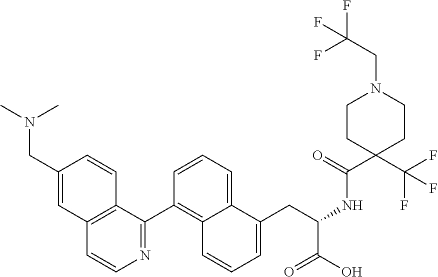 [SMILES: CN(C)Cc1ccc2c(-c3cccc4c(C[C@H](NC(=O)C5(C(F)(F)F)CCN(CC(F)(F)F)CC5)C(=O)O)cccc34)nccc2c1] |
| | m/z = 661.4 |
| 585** | 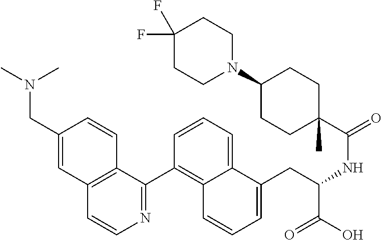 [SMILES: CN(C)Cc1ccc2c(-c3cccc4c(C[C@H](NC(=O)[C@]5(C)CC[C@@H](N6CCC(F)(F)CC6)CC5)C(=O)O)cccc34)nccc2c1] |
| | m/z = 643.5 |
| 586** | 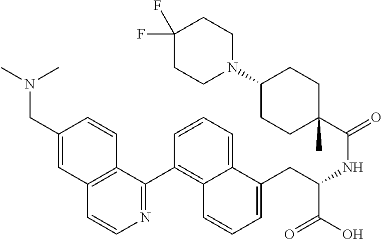 [SMILES: CN(C)Cc1ccc2c(-c3cccc4c(C[C@H](NC(=O)[C@]5(C)CC[C@H](N6CCC(F)(F)CC6)CC5)C(=O)O)cccc34)nccc2c1] |
| | m/z = 643.4 |
| 587 | 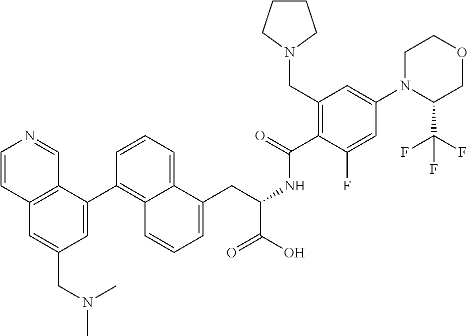 [SMILES: CN(C)Cc1cc(-c2cccc3c(C[C@H](NC(=O)c4c(F)cc(N5CCOC[C@@H]5C(F)(F)F)cc4CN4CCCC4)C(=O)O)cccc23)c2cnccc2c1] |
| | m/z = 757.8 |
| 588 | 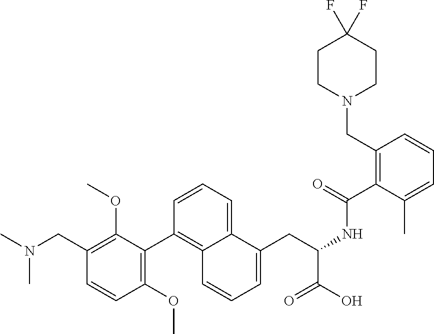 [SMILES: COc1ccc(CN(C)C)c(OC)c1-c1cccc2c(C[C@H](NC(=O)c3c(C)cccc3CN3CCC(F)(F)CC3)C(=O)O)cccc12] |
| | m/z = 660.5 |
| 589 | 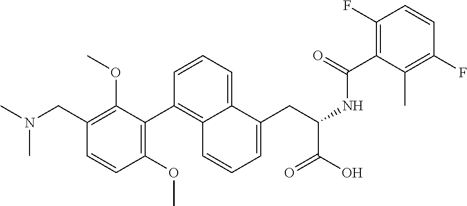 [SMILES: COc1ccc(CN(C)C)c(OC)c1-c1cccc2c(C[C@H](NC(=O)c3c(F)ccc(F)c3C)C(=O)O)cccc12] |
| | m/z = 563.5 |
| 590 | 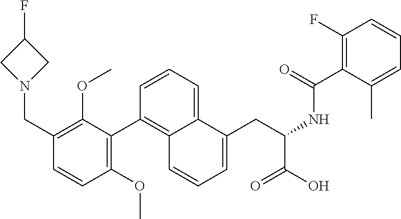 [SMILES: COc1ccc(CN2CC(F)C2)c(OC)c1-c1cccc2c(C[C@H](NC(=O)c3c(C)cccc3F)C(=O)O)cccc12] |
| | m/z = 575.2 |
| 591 | 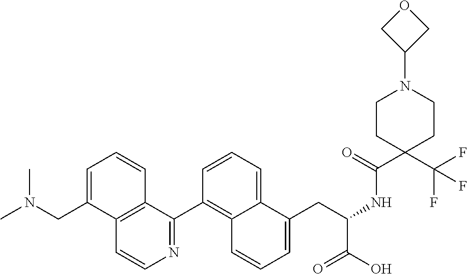 [SMILES: CN(C)Cc1cccc2c(-c3cccc4c(C[C@H](NC(=O)C5(C(F)(F)F)CCN(C6COC6)CC5)C(=O)O)cccc34)nccc12] |
| | m/z = 635.2 |
| 592** | 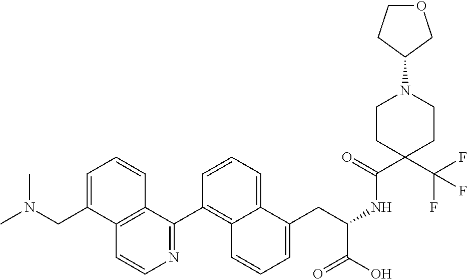 [SMILES: CN(C)Cc1cccc2c(-c3cccc4c(C[C@H](NC(=O)C5(C(F)(F)F)CCN([C@@H]6CCOC6)CC5)C(=O)O)cccc34)nccc12] |
| | m/z = 649.2 |
| 593** | 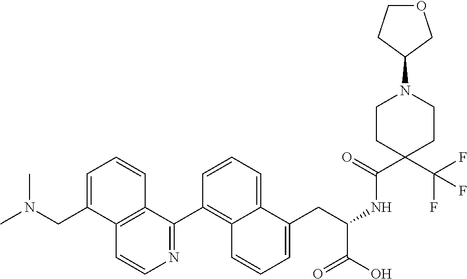 [SMILES: CN(C)Cc1cccc2c(-c3cccc4c(C[C@H](NC(=O)C5(C(F)(F)F)CCN([C@H]6CCOC6)CC5)C(=O)O)cccc34)nccc12] |
| | m/z = 649.3 |
| 594 | 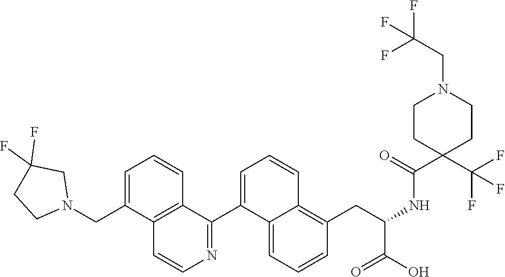 [SMILES: O=C(O)[C@H](Cc1cccc2c(-c3nccc4c(CN5CCC(F)(F)C5)cccc34)cccc12)NC(=O)C1(C(F)(F)F)CCN(CC(F)(F)F)CC1] |
| | m/z = 723.2 |
| 595 | 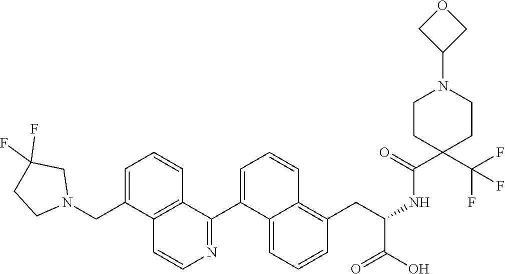 [SMILES: O=C(O)[C@H](Cc1cccc2c(-c3nccc4c(CN5CCC(F)(F)C5)cccc34)cccc12)NC(=O)C1(C(F)(F)F)CCN(C2COC2)CC1] |
| | m/z = 697.2 |
| 596 | 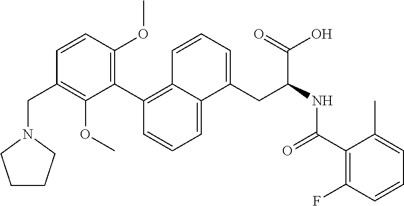 [SMILES: COc1ccc(CN2CCCC2)c(OC)c1-c1cccc2c(C[C@H](NC(=O)c3c(C)cccc3F)C(=O)O)cccc12] |
| | m/z = 571.1 |
| 597 | 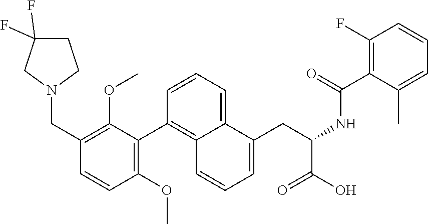 [SMILES: COc1ccc(CN2CCC(F)(F)C2)c(OC)c1-c1cccc2c(C[C@H](NC(=O)c3c(C)cccc3F)C(=O)O)cccc12] |
| | m/z = 607.1 |
| 598 | 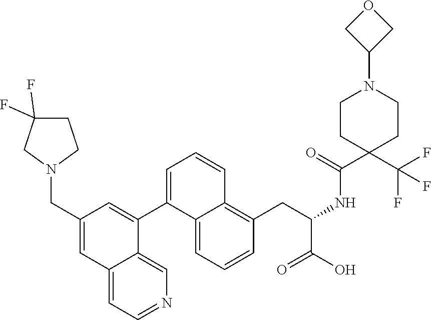 [SMILES: O=C(O)[C@H](Cc1cccc2c(-c3cc(CN4CCC(F)(F)C4)cc4ccncc34)cccc12)NC(=O)C1(C(F)(F)F)CCN(C2COC2)CC1] |
| | m/z = 697.2 |
| 599 | 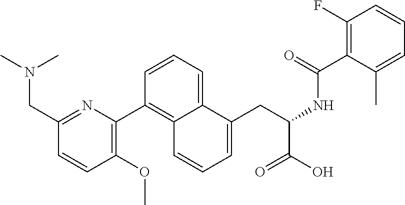 [SMILES: COc1ccc(CN(C)C)nc1-c1cccc2c(C[C@H](NC(=O)c3c(C)cccc3F)C(=O)O)cccc12] |
| | m/z = 516.2 |
| 600 | 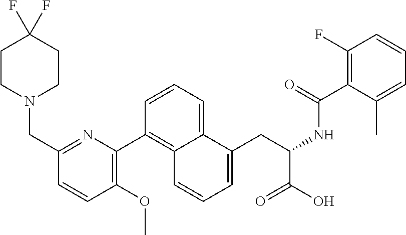 [SMILES: COc1ccc(CN2CCC(F)(F)CC2)nc1-c1cccc2c(C[C@H](NC(=O)c3c(C)cccc3F)C(=O)O)cccc12] |
| | m/z = 592.1 |
| 601 | 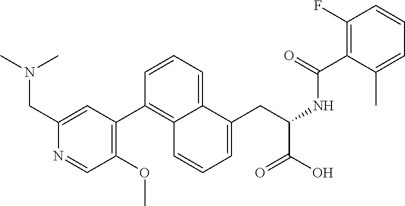 [SMILES: COc1cnc(CN(C)C)cc1-c1cccc2c(C[C@H](NC(=O)c3c(C)cccc3F)C(=O)O)cccc12] |
| | m/z = 516.1 |
| 602 | 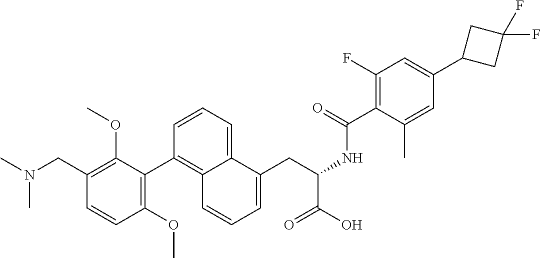 [SMILES: COc1ccc(CN(C)C)c(OC)c1-c1cccc2c(C[C@H](NC(=O)c3c(C)cc(C4CC(F)(F)C4)cc3F)C(=O)O)cccc12] |
| | m/z = 635.3 |
| 603 | 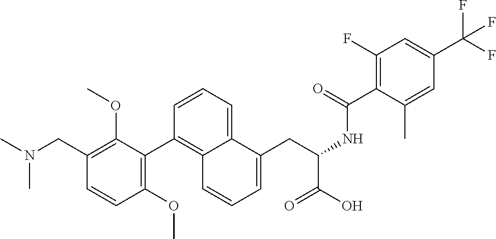 [SMILES: COc1ccc(CN(C)C)c(OC)c1-c1cccc2c(C[C@H](NC(=O)c3c(C)cc(C(F)(F)F)cc3F)C(=O)O)cccc12] |
| | m/z = 613.1 |
| 604 | 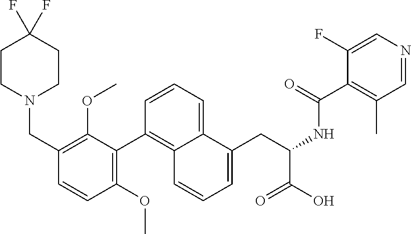 [SMILES: COc1ccc(CN2CCC(F)(F)CC2)c(OC)c1-c1cccc2c(C[C@H](NC(=O)c3c(C)cncc3F)C(=O)O)cccc12] |
| | m/z = 622.1 |
| 605 | 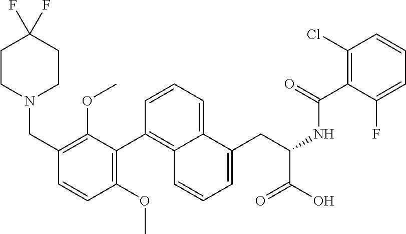 [SMILES: COc1ccc(CN2CCC(F)(F)CC2)c(OC)c1-c1cccc2c(C[C@H](NC(=O)c3c(F)cccc3Cl)C(=O)O)cccc12] |
| | m/z = 641.3 |
| 606 | 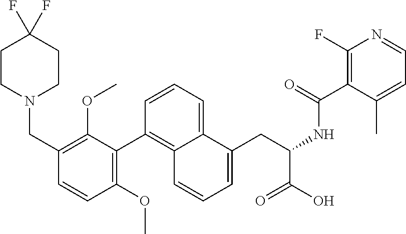 [SMILES: COc1ccc(CN2CCC(F)(F)CC2)c(OC)c1-c1cccc2c(C[C@H](NC(=O)c3c(C)ccnc3F)C(=O)O)cccc12] |
| | m/z = 622.2 |
| 607 | 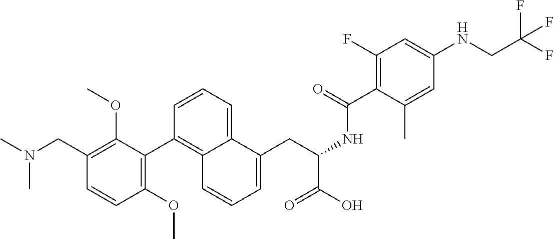 [SMILES: COc1ccc(CN(C)C)c(OC)c1-c1cccc2c(C[C@H](NC(=O)c3c(C)cc(NCC(F)(F)F)cc3F)C(=O)O)cccc12] |
| | m/z = 642.1 |
| 608 | 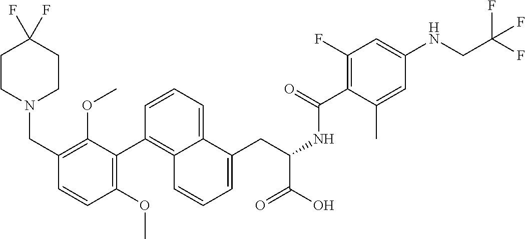 [SMILES: COc1ccc(CN2CCC(F)(F)CC2)c(OC)c1-c1cccc2c(C[C@H](NC(=O)c3c(C)cc(NCC(F)(F)F)cc3F)C(=O)O)cccc12] |
| | m/z = 718.4 |
| 609 | 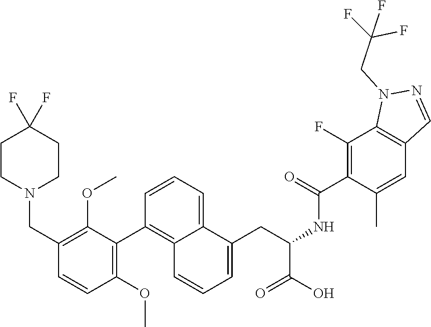 [SMILES: COc1ccc(CN2CCC(F)(F)CC2)c(OC)c1-c1cccc2c(C[C@H](NC(=O)c3c(C)cc4cnn(CC(F)(F)F)c4c3F)C(=O)O)cccc12] |
| | m/z = 743.3 |
| 610 | 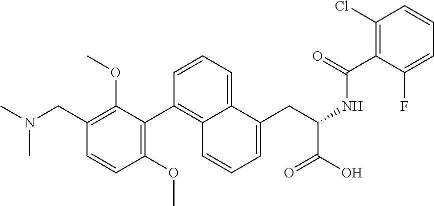 [SMILES: COc1ccc(CN(C)C)c(OC)c1-c1cccc2c(C[C@H](NC(=O)c3c(F)cccc3Cl)C(=O)O)cccc12] |
| | m/z = 565 |
| 611 | 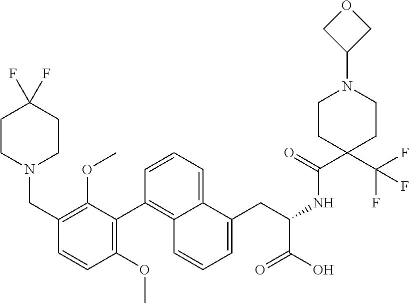 [SMILES: COc1ccc(CN2CCC(F)(F)CC2)c(OC)c1-c1cccc2c(C[C@H](NC(=O)C3(C(F)(F)F)CCN(C4COC4)CC3)C(=O)O)cccc12] |
| | m/z = 720.3 |
| 612 | 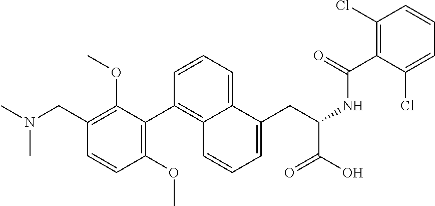 [SMILES: COc1ccc(CN(C)C)c(OC)c1-c1cccc2c(C[C@H](NC(=O)c3c(Cl)cccc3Cl)C(=O)O)cccc12] |
| | m/z = 581 |
| 613 | 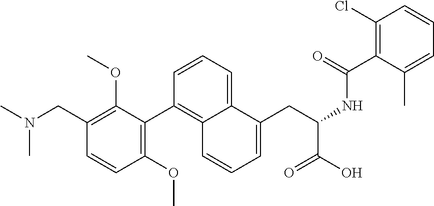 [SMILES: COc1ccc(CN(C)C)c(OC)c1-c1cccc2c(C[C@H](NC(=O)c3c(C)cccc3Cl)C(=O)O)cccc12] |
| | m/z = 561 |
| 614 | 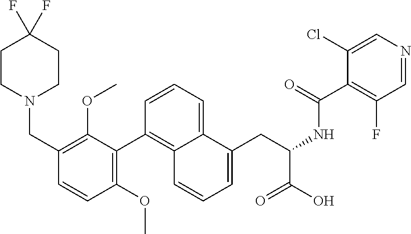 [SMILES: COc1ccc(CN2CCC(F)(F)CC2)c(OC)c1-c1cccc2c(C[C@H](NC(=O)c3c(F)cncc3Cl)C(=O)O)cccc12] |
| | m/z = 642.1 |
| 615 | 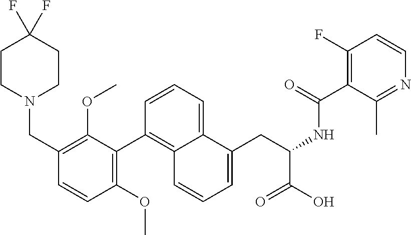 [SMILES: COc1ccc(CN2CCC(F)(F)CC2)c(OC)c1-c1cccc2c(C[C@H](NC(=O)c3c(F)ccnc3C)C(=O)O)cccc12] |
| | m/z = 622.2 |
| 616* | 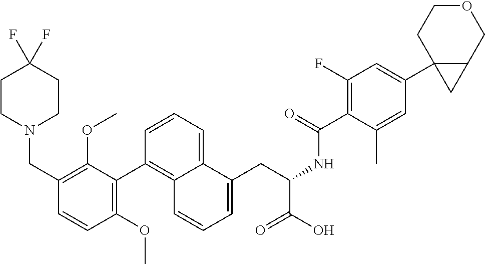 [SMILES: COc1ccc(CN2CCC(F)(F)CC2)c(OC)c1-c1cccc2c(C[C@H](NC(=O)c3c(C)cc(C45CCOCC4C5)cc3F)C(=O)O)cccc12] |
| | m/z = 717.5 |
| 617 | 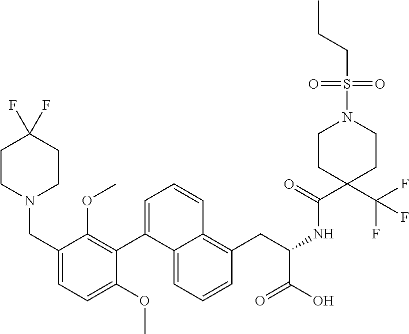 [SMILES: CCCS(=O)(=O)N1CCC(C(=O)N[C@@H](Cc2cccc3c(-c4c(OC)ccc(CN5CCC(F)(F)CC5)c4OC)cccc23)C(=O)O)(C(F)(F)F)CC1] |
| | m/z = 770 |
| 618 | 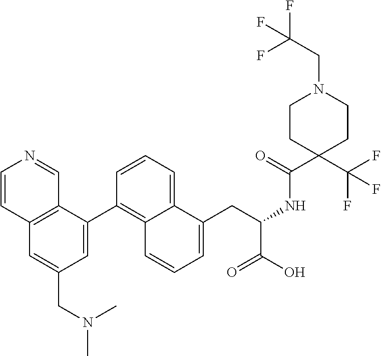 [SMILES: CN(C)Cc1cc(-c2cccc3c(C[C@H](NC(=O)C4(C(F)(F)F)CCN(CC(F)(F)F)CC4)C(=O)O)cccc23)c2cnccc2c1] |
| | m/z = 661.2 |
| 619 | 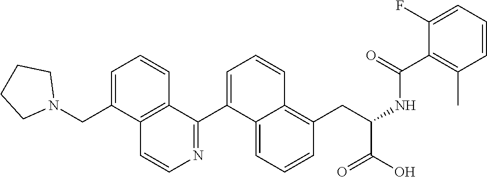 [SMILES: Cc1cccc(F)c1C(=O)N[C@@H](Cc1cccc2c(-c3nccc4c(CN5CCCC5)cccc34)cccc12)C(=O)O] |
| | m/z = 562.3 |
| 620 | 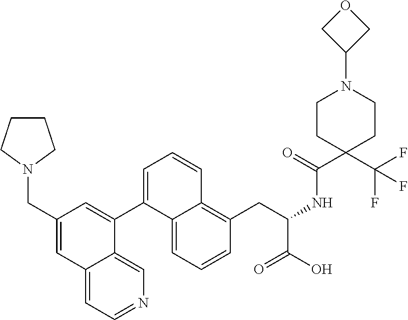 [SMILES: O=C(O)[C@H](Cc1cccc2c(-c3cc(CN4CCCC4)cc4ccncc34)cccc12)NC(=O)C1(C(F)(F)F)CCN(C2COC2)CC1] |
| | m/z = 661.2 |
| 621 | 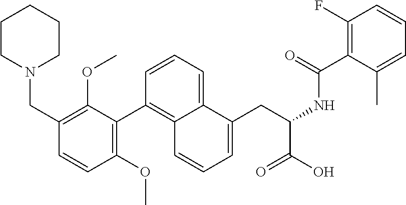 [SMILES: COc1ccc(CN2CCCCC2)c(OC)c1-c1cccc2c(C[C@H](NC(=O)c3c(C)cccc3F)C(=O)O)cccc12] |
| | m/z = 585.3 |
| 622 | 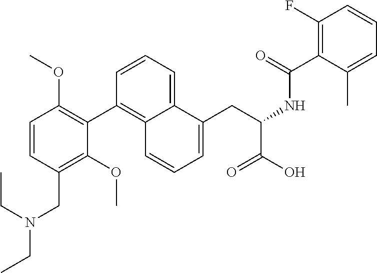 [SMILES: CCN(CC)Cc1ccc(OC)c(-c2cccc3c(C[C@H](NC(=O)c4c(C)cccc4F)C(=O)O)cccc23)c1OC] |
| | m/z = 573.3 |
| 623 | 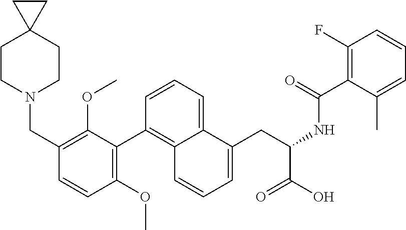 [SMILES: COc1ccc(CN2CCC3(CC2)CC3)c(OC)c1-c1cccc2c(C[C@H](NC(=O)c3c(C)cccc3F)C(=O)O)cccc12] |
| | m/z = 611.2 |
| 624 | 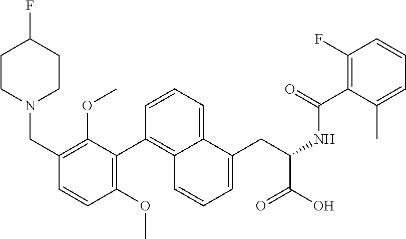 [SMILES: COc1ccc(CN2CCC(F)CC2)c(OC)c1-c1cccc2c(C[C@H](NC(=O)c3c(C)cccc3F)C(=O)O)cccc12] |
| | m/z = 603.3 |
| 625 | 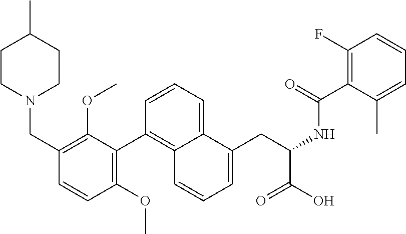 [SMILES: COc1ccc(CN2CCC(C)CC2)c(OC)c1-c1cccc2c(C[C@H](NC(=O)c3c(C)cccc3F)C(=O)O)cccc12] |
| | m/z = 599.3 |
| 626 | 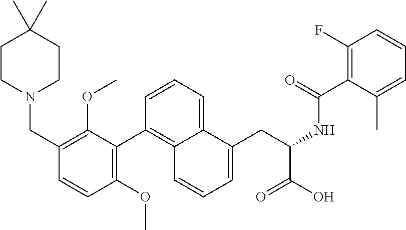 [SMILES: COc1ccc(CN2CCC(C)(C)CC2)c(OC)c1-c1cccc2c(C[C@H](NC(=O)c3c(C)cccc3F)C(=O)O)cccc12] |
| | m/z = 613.4 |
| 627 | 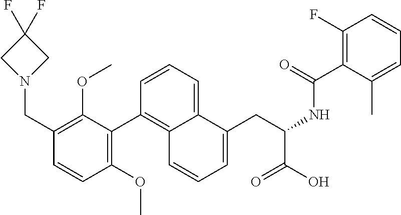 [SMILES: COc1ccc(CN2CC(F)(F)C2)c(OC)c1-c1cccc2c(C[C@H](NC(=O)c3c(C)cccc3F)C(=O)O)cccc12] |
| | m/z = 593.2 |
| 628 | 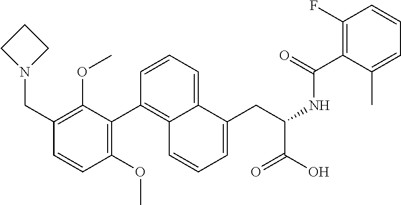 [SMILES: COc1ccc(CN2CCC2)c(OC)c1-c1cccc2c(C[C@H](NC(=O)c3c(C)cccc3F)C(=O)O)cccc12] |
| | m/z = 557.2 |
| 629* | 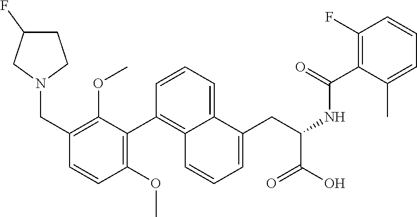 [SMILES: COc1ccc(CN2CCC(F)C2)c(OC)c1-c1cccc2c(C[C@H](NC(=O)c3c(C)cccc3F)C(=O)O)cccc12] |
| | m/z = 589.3 |
| 630 | 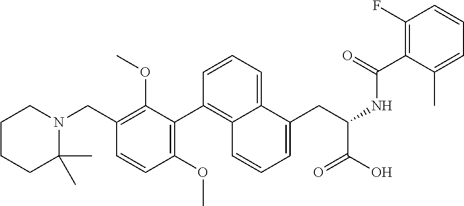 [SMILES: COc1ccc(CN2CCCCC2(C)C)c(OC)c1-c1cccc2c(C[C@H](NC(=O)c3c(C)cccc3F)C(=O)O)cccc12] |
| | m/z = 613.2 |
| 631* | 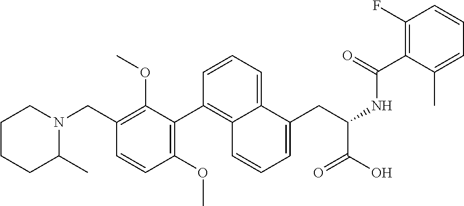 [SMILES: COc1ccc(CN2CCCCC2C)c(OC)c1-c1cccc2c(C[C@H](NC(=O)c3c(C)cccc3F)C(=O)O)cccc12] |
| | m/z = 599.4 |
| 632 | 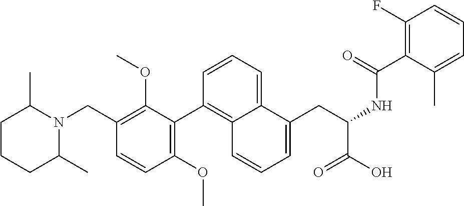 [SMILES: COc1ccc(CN2C(C)CCCC2C)c(OC)c1-c1cccc2c(C[C@H](NC(=O)c3c(C)cccc3F)C(=O)O)cccc12] |
| | m/z = 613.2 |
| 633* | 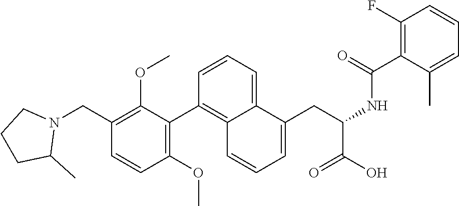 [SMILES: COc1ccc(CN2CCCC2C)c(OC)c1-c1cccc2c(C[C@H](NC(=O)c3c(C)cccc3F)C(=O)O)cccc12] |
| | m/z = 585.2 |
| 634 | 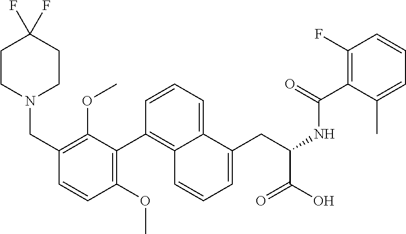 [SMILES: COc1ccc(CN2CCC(F)(F)CC2)c(OC)c1-c1cccc2c(C[C@H](NC(=O)c3c(C)cccc3F)C(=O)O)cccc12] |
| | m/z = 621.4 |
| 635 | 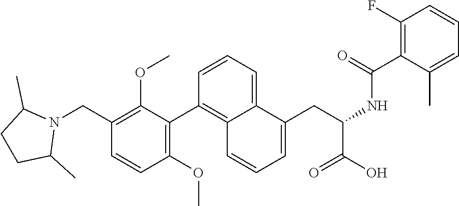 [SMILES: COc1ccc(CN2C(C)CCC2C)c(OC)c1-c1cccc2c(C[C@H](NC(=O)c3c(C)cccc3F)C(=O)O)cccc12] |
| | m/z = 613.2 |
| 636 | 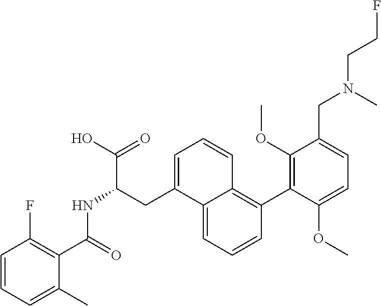 [SMILES: COc1ccc(CN(C)CCF)c(OC)c1-c1cccc2c(C[C@H](NC(=O)c3c(C)cccc3F)C(=O)O)cccc12] |
| | m/z = 577.2 |
| 637 | 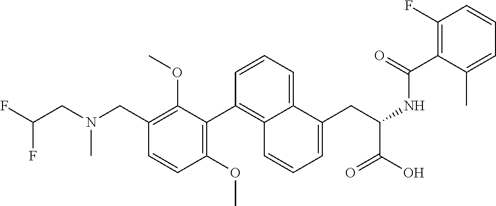 [SMILES: COc1ccc(CN(C)CC(F)F)c(OC)c1-c1cccc2c(C[C@H](NC(=O)c3c(C)cccc3F)C(=O)O)cccc12] |
| | m/z = 595.3 |
| 638 | 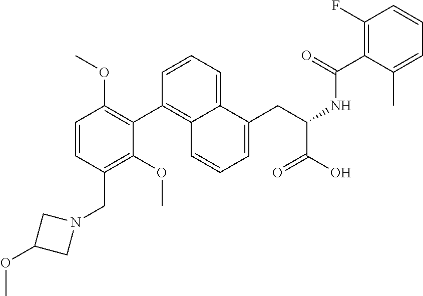 [SMILES: COc1ccc(CN2CC(OC)C2)c(OC)c1-c1cccc2c(C[C@H](NC(=O)c3c(C)cccc3F)C(=O)O)cccc12] |
| | m/z = 587.3 |
| 639** | 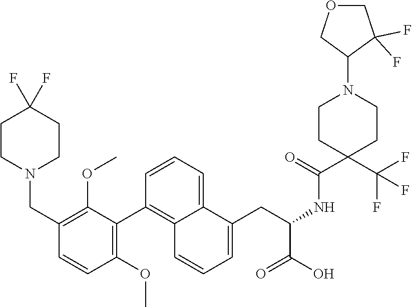 [SMILES: COc1ccc(CN2CCC(F)(F)CC2)c(OC)c1-c1cccc2c(C[C@H](NC(=O)C3(C(F)(F)F)CCN(C4COCC4(F)F)CC3)C(=O)O)cccc12] |
| | m/z = 770.2 |
| 640** | 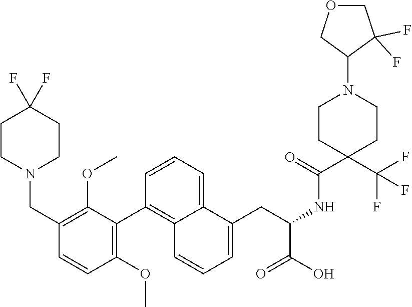 [SMILES: COc1ccc(CN2CCC(F)(F)CC2)c(OC)c1-c1cccc2c(C[C@H](NC(=O)C3(C(F)(F)F)CCN(C4COCC4(F)F)CC3)C(=O)O)cccc12] |
| | m/z = 770.2 |
| 641** | 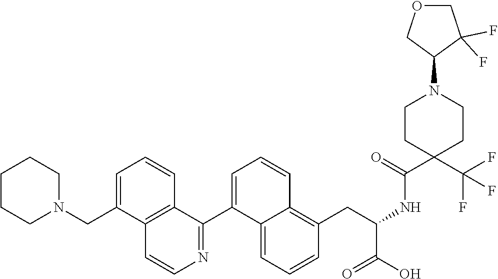 [SMILES: O=C(O)[C@H](Cc1cccc2c(-c3nccc4c(CN5CCCCC5)cccc34)cccc12)NC(=O)C1(C(F)(F)F)CCN([C@H]2COCC2(F)F)CC1] |
| | m/z = 725.2 |
| 642** | 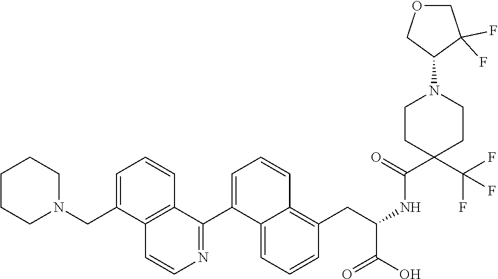 [SMILES: O=C(O)[C@H](Cc1cccc2c(-c3nccc4c(CN5CCCCC5)cccc34)cccc12)NC(=O)C1(C(F)(F)F)CCN([C@@H]2COCC2(F)F)CC1] |
| | m/z = 725.3 |
| 643* | 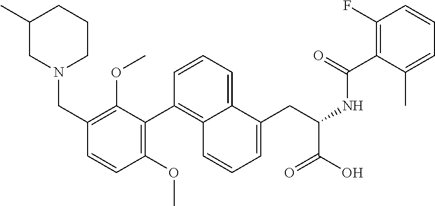 [SMILES: COc1ccc(CN2CCCC(C)C2)c(OC)c1-c1cccc2c(C[C@H](NC(=O)c3c(C)cccc3F)C(=O)O)cccc12] |
| | m/z = 599.3 |
| 644* | 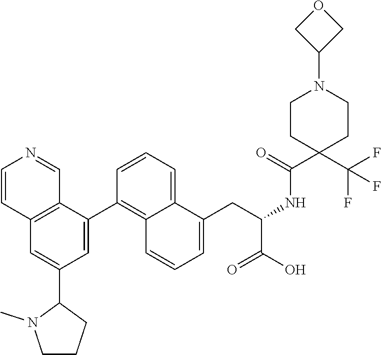 [SMILES: CN1CCCC1c1cc(-c2cccc3c(C[C@H](NC(=O)C4(C(F)(F)F)CCN(C5COC5)CC4)C(=O)O)cccc23)c2cnccc2c1] |
| | m/z = 661.4 |
| 645 | 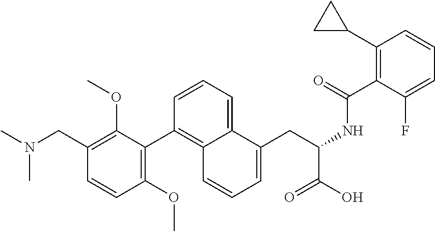 [SMILES: COc1ccc(CN(C)C)c(OC)c1-c1cccc2c(C[C@H](NC(=O)c3c(F)cccc3C3CC3)C(=O)O)cccc12] |
| | m/z = 571.3 |
| 646 | 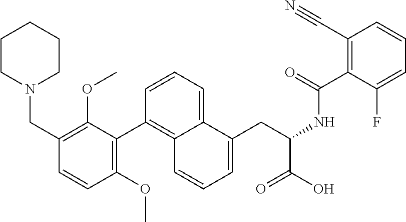 [SMILES: COc1ccc(CN2CCCCC2)c(OC)c1-c1cccc2c(C[C@H](NC(=O)c3c(F)cccc3C#N)C(=O)O)cccc12] |
| | m/z = 596.5 |
| 647 | 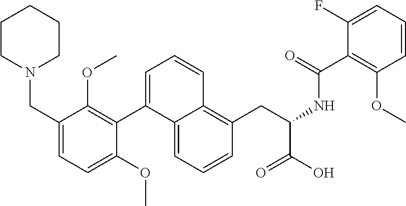 [SMILES: COc1cccc(F)c1C(=O)N[C@@H](Cc1cccc2c(-c3c(OC)ccc(CN4CCCCC4)c3OC)cccc12)C(=O)O] |
| | m/z = 601.2 |
| 648 | 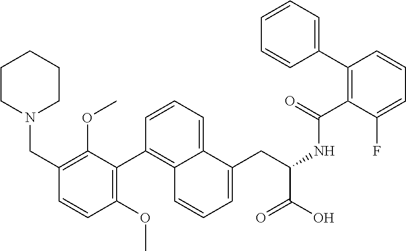 [SMILES: COc1ccc(CN2CCCCC2)c(OC)c1-c1cccc2c(C[C@H](NC(=O)c3c(F)cccc3-c3ccccc3)C(=O)O)cccc12] |
| | m/z = 647.3 |
| 649 | 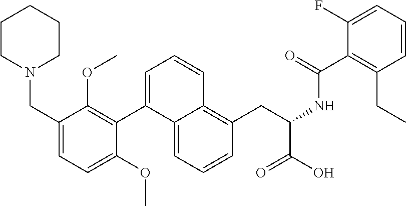 [SMILES: CCc1cccc(F)c1C(=O)N[C@@H](Cc1cccc2c(-c3c(OC)ccc(CN4CCCCC4)c3OC)cccc12)C(=O)O] |
| | m/z = 599.4 |
| 650 | 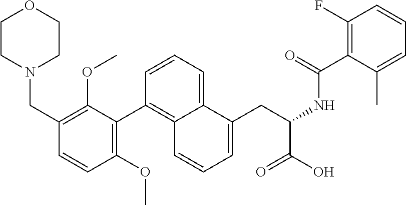 [SMILES: COc1ccc(CN2CCOCC2)c(OC)c1-c1cccc2c(C[C@H](NC(=O)c3c(C)cccc3F)C(=O)O)cccc12] |
| | m/z = 587.2 |
| 651 | 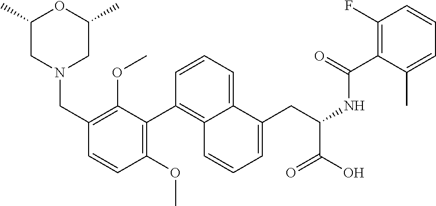 [SMILES: COc1ccc(CN2C[C@@H](C)O[C@@H](C)C2)c(OC)c1-c1cccc2c(C[C@H](NC(=O)c3c(C)cccc3F)C(=O)O)cccc12] |
| | m/z = 615.4 |
| 652 | 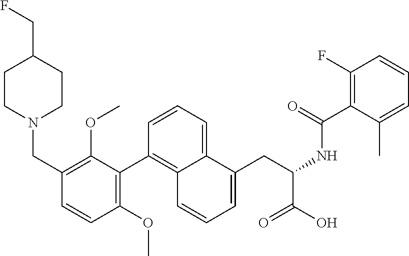 [SMILES: COc1ccc(CN2CCC(CF)CC2)c(OC)c1-c1cccc2c(C[C@H](NC(=O)c3c(C)cccc3F)C(=O)O)cccc12] |
| | m/z = 617.5 |
| 653 | 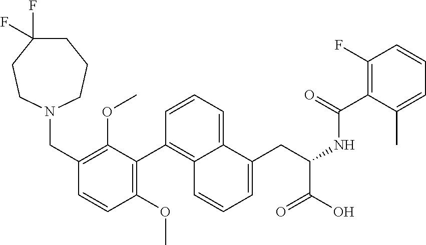 [SMILES: COc1ccc(CN2CCCC(F)(F)CC2)c(OC)c1-c1cccc2c(C[C@H](NC(=O)c3c(C)cccc3F)C(=O)O)cccc12] |
| | m/z = 635.2 |
| 654 | 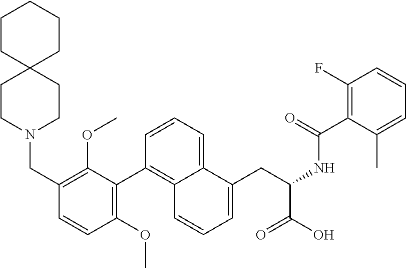 [SMILES: COc1ccc(CN2CCC3(CCCCC3)CC2)c(OC)c1-c1cccc2c(C[C@H](NC(=O)c3c(C)cccc3F)C(=O)O)cccc12] |
| | m/z = 653.2 |
| 655 | 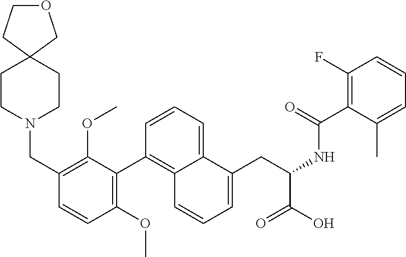 [SMILES: COc1ccc(CN2CCC3(CCOC3)CC2)c(OC)c1-c1cccc2c(C[C@H](NC(=O)c3c(C)cccc3F)C(=O)O)cccc12] |
| | m/z = 641.4 |
| 656 | 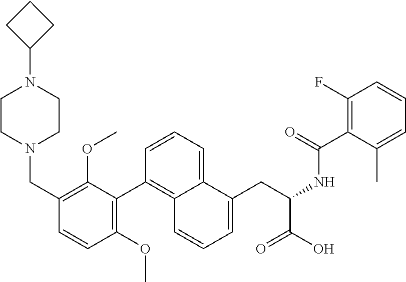 [SMILES: COc1ccc(CN2CCN(C3CCC3)CC2)c(OC)c1-c1cccc2c(C[C@H](NC(=O)c3c(C)cccc3F)C(=O)O)cccc12] |
| | m/z = 640.3 |
| 657 | 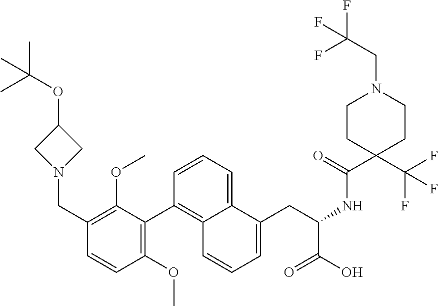 [SMILES: COc1ccc(CN2CC(OC(C)(C)C)C2)c(OC)c1-c1cccc2c(C[C@H](NC(=O)C3(C(F)(F)F)CCN(CC(F)(F)F)CC3)C(=O)O)cccc12] |
| | m/z = 754.4 |
| 658 | 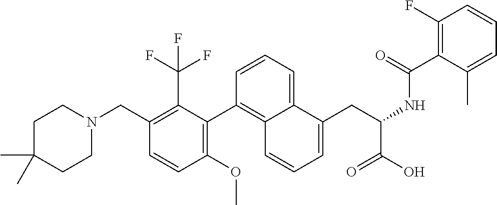 [SMILES: COc1ccc(CN2CCC(C)(C)CC2)c(C(F)(F)F)c1-c1cccc2c(C[C@H](NC(=O)c3c(C)cccc3F)C(=O)O)cccc12] |
| | m/z = 651.2 |
| 659 | 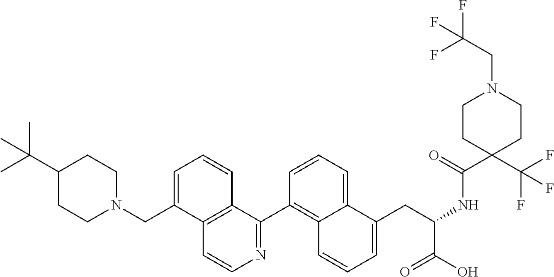 [SMILES: CC(C)(C)C1CCN(Cc2cccc3c(-c4cccc5c(C[C@H](NC(=O)C6(C(F)(F)F)CCN(CC(F)(F)F)CC6)C(=O)O)cccc45)nccc23)CC1] |
| | m/z = 757.2 |
| 660* | 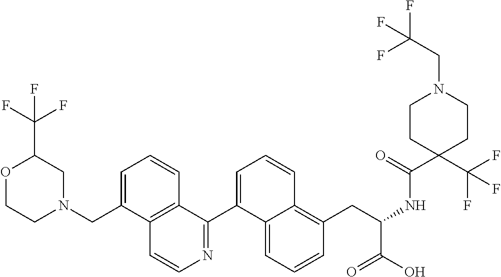 [SMILES: O=C(O)[C@H](Cc1cccc2c(-c3nccc4c(CN5CCOC(C(F)(F)F)C5)cccc34)cccc12)NC(=O)C1(C(F)(F)F)CCN(CC(F)(F)F)CC1] |
| | m/z = 771.1 |
| 661 | 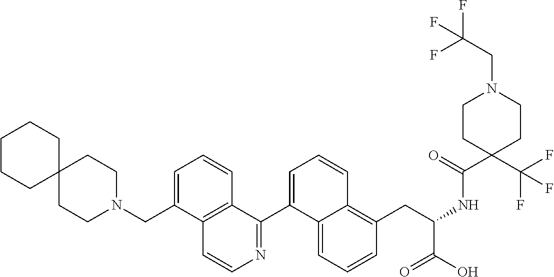 [SMILES: O=C(O)[C@H](Cc1cccc2c(-c3nccc4c(CN5CCC6(CCCCC6)CC5)cccc34)cccc12)NC(=O)C1(C(F)(F)F)CCN(CC(F)(F)F)CC1] |
| | m/z = 769.2 |
| 662 | 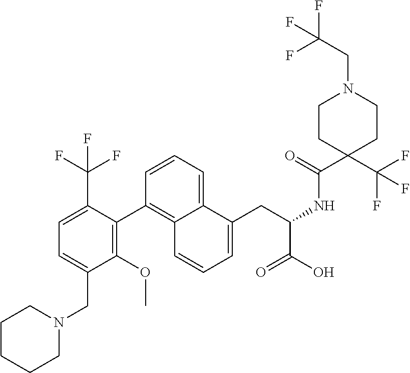 [SMILES: COc1c(CN2CCCCC2)ccc(C(F)(F)F)c1-c1cccc2c(C[C@H](NC(=O)C3(C(F)(F)F)CCN(CC(F)(F)F)CC3)C(=O)O)cccc12] |
| | m/z = 748.1 |
| 663* | 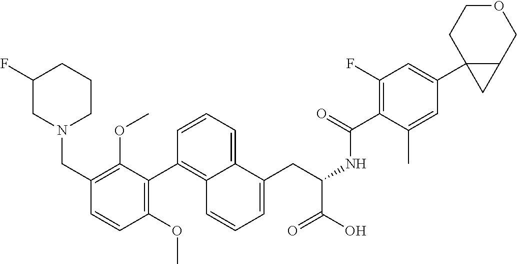 [SMILES: COc1ccc(CN2CCCC(F)C2)c(OC)c1-c1cccc2c(C[C@H](NC(=O)c3c(C)cc(C45CCOCC4C5)cc3F)C(=O)O)cccc12] |
| | m/z = 699.2 |
| 664 | 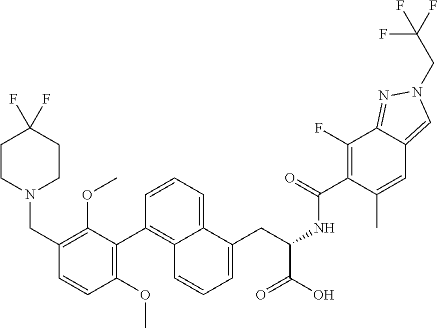 [SMILES: COc1ccc(CN2CCC(F)(F)CC2)c(OC)c1-c1cccc2c(C[C@H](NC(=O)c3c(C)cc4cn(CC(F)(F)F)nc4c3F)C(=O)O)cccc12] |
| | m/z = 743.2 |
| 665 | 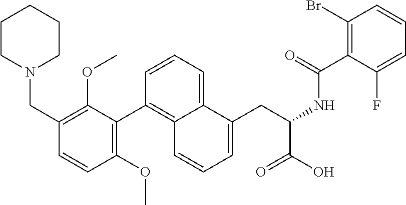 [SMILES: COc1ccc(CN2CCCCC2)c(OC)c1-c1cccc2c(C[C@H](NC(=O)c3c(F)cccc3Br)C(=O)O)cccc12] |
| | m/z = 649.3 |
| 666 | 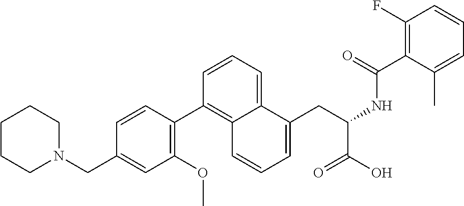 [SMILES: COc1cc(CN2CCCCC2)ccc1-c1cccc2c(C[C@H](NC(=O)c3c(C)cccc3F)C(=O)O)cccc12] |
| | m/z = 555.3 |
| 667 | 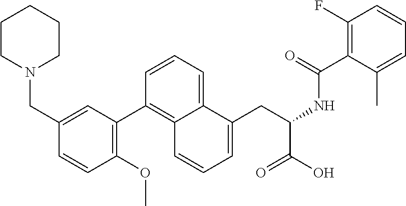 [SMILES: COc1ccc(CN2CCCCC2)cc1-c1cccc2c(C[C@H](NC(=O)c3c(C)cccc3F)C(=O)O)cccc12] |
| | m/z = 555.3 |
| 668** | 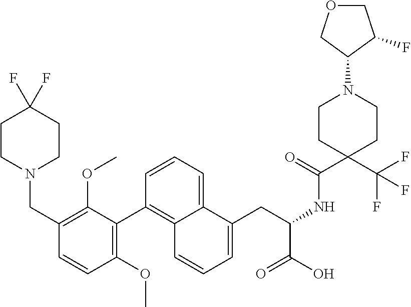 [SMILES: COc1ccc(CN2CCC(F)(F)CC2)c(OC)c1-c1cccc2c(C[C@H](NC(=O)C3(C(F)(F)F)CCN([C@@H]4COC[C@@H]4F)CC3)C(=O)O)cccc12] |
| | m/z = 752.5 |
| 669** | 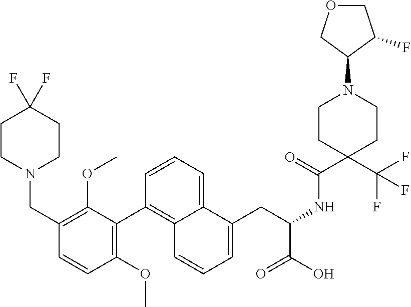 [SMILES: COc1ccc(CN2CCC(F)(F)CC2)c(OC)c1-c1cccc2c(C[C@H](NC(=O)C3(C(F)(F)F)CCN([C@H]4COC[C@@H]4F)CC3)C(=O)O)cccc12] |
| | m/z = 752.2 |
| 670** | 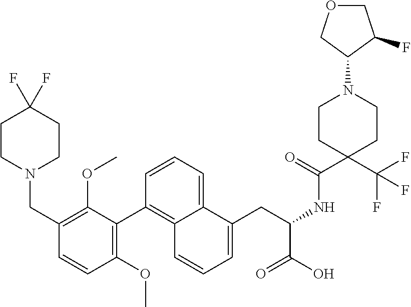 [SMILES: COc1ccc(CN2CCC(F)(F)CC2)c(OC)c1-c1cccc2c(C[C@H](NC(=O)C3(C(F)(F)F)CCN([C@@H]4COC[C@H]4F)CC3)C(=O)O)cccc12] |
| | m/z = 752.2 |
| 671 | 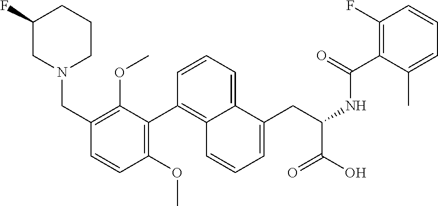 [SMILES: COc1ccc(CN2CCC[C@H](F)C2)c(OC)c1-c1cccc2c(C[C@H](NC(=O)c3c(C)cccc3F)C(=O)O)cccc12] |
| | m/z = 603.2 |
| 672 | 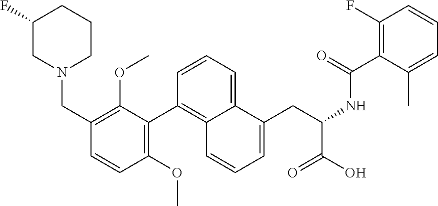 [SMILES: COc1ccc(CN2CCC[C@@H](F)C2)c(OC)c1-c1cccc2c(C[C@H](NC(=O)c3c(C)cccc3F)C(=O)O)cccc12] |
| | m/z = 603.2 |
| 673 | 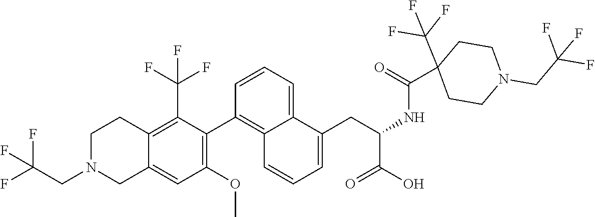 [SMILES: COc1cc2c(c(C(F)(F)F)c1-c1cccc3c(C[C@H](NC(=O)C4(C(F)(F)F)CCN(CC(F)(F)F)CC4)C(=O)O)cccc13)CCN(CC(F)(F)F)C2] |
| | m/z = 788.4 |
| 674 | 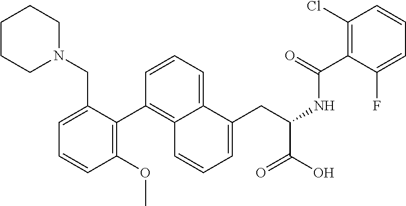 [SMILES: COc1cccc(CN2CCCCC2)c1-c1cccc2c(C[C@H](NC(=O)c3c(F)cccc3Cl)C(=O)O)cccc12] |
| | m/z = 575.3 |
| 675 | 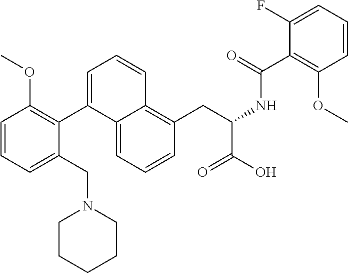 |
| | m/z = 571.4 |
| 676 | 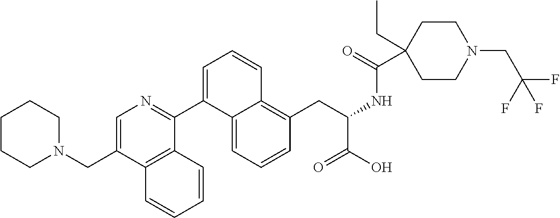 [SMILES: CCC1(C(=O)N[C@@H](Cc2cccc3c(-c4ncc(CN5CCCCC5)c5ccccc45)cccc23)C(=O)O)CCN(CC(F)(F)F)CC1] |
| | m/z = 661.4 |
| 677 | 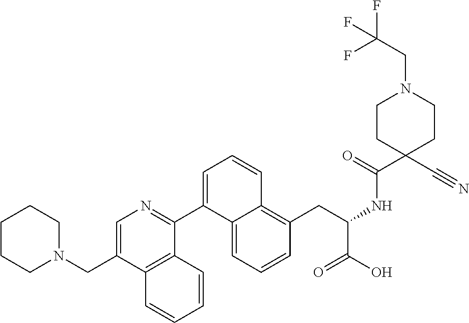 [SMILES: N#CC1(C(=O)N[C@@H](Cc2cccc3c(-c4ncc(CN5CCCCC5)c5ccccc45)cccc23)C(=O)O)CCN(CC(F)(F)F)CC1] |
| | m/z = 658.2 |
| 678 | 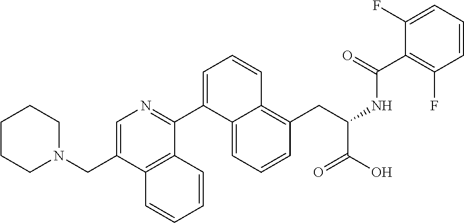 [SMILES: O=C(N[C@@H](Cc1cccc2c(-c3ncc(CN4CCCCC4)c4ccccc34)cccc12)C(=O)O)c1c(F)cccc1F] |
| | m/z = 580.2 |
| 679* | 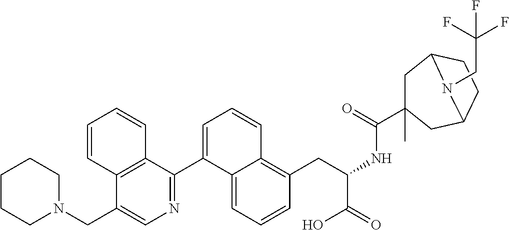 [SMILES: CC1(C(=O)N[C@@H](Cc2cccc3c(-c4ncc(CN5CCCCC5)c5ccccc45)cccc23)C(=O)O)CC2CCC(C1)N2CC(F)(F)F] |
| | m/z = 673.3 |
| 680** | 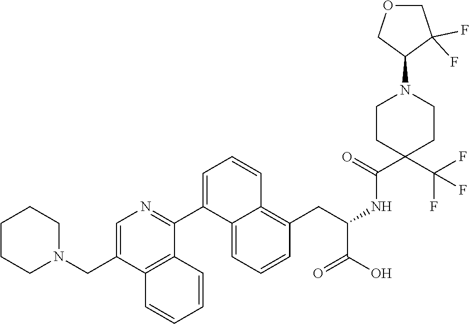 [SMILES: O=C(O)[C@H](Cc1cccc2c(-c3ncc(CN4CCCCC4)c4ccccc34)cccc12)NC(=O)C1(C(F)(F)F)CCN([C@H]2COCC2(F)F)CC1] |
| | m/z = 725.4 |
| 681 | 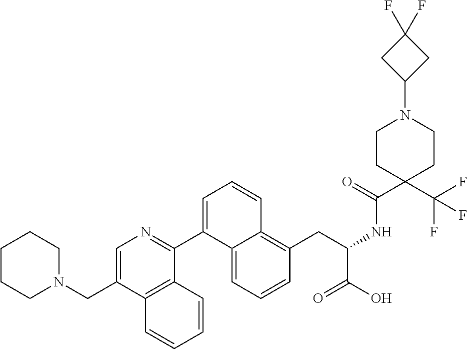 [SMILES: O=C(O)[C@H](Cc1cccc2c(-c3ncc(CN4CCCCC4)c4ccccc34)cccc12)NC(=O)C1(C(F)(F)F)CCN(C2CC(F)(F)C2)CC1] |
| | m/z = 709.2 |
| 682 | 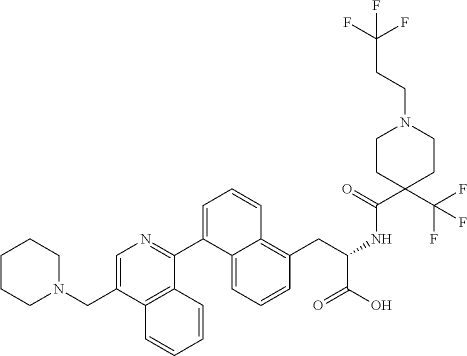 [SMILES: O=C(O)[C@H](Cc1cccc2c(-c3ncc(CN4CCCCC4)c4ccccc34)cccc12)NC(=O)C1(C(F)(F)F)CCN(CCC(F)(F)F)CC1] |
| | m/z = 715.3 |
| 683 | 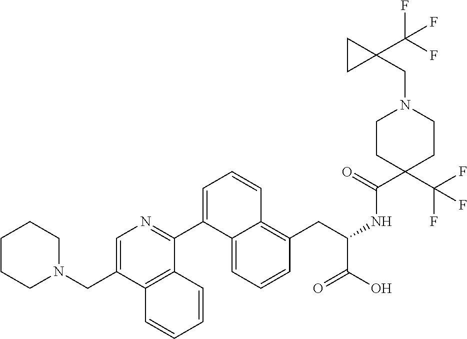 [SMILES: O=C(O)[C@H](Cc1cccc2c(-c3ncc(CN4CCCCC4)c4ccccc34)cccc12)NC(=O)C1(C(F)(F)F)CCN(CC2(C(F)(F)F)CC2)CC1] |
| | m/z = 741.2 |
| 684* | 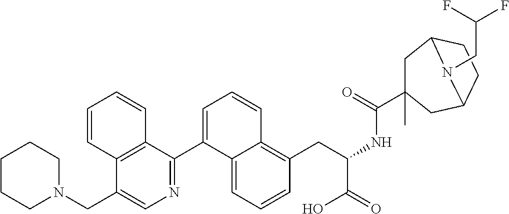 [SMILES: CC1(C(=O)N[C@@H](Cc2cccc3c(-c4ncc(CN5CCCCC5)c5ccccc45)cccc23)C(=O)O)CC2CCC(C1)N2CC(F)F] |
| | m/z = 655.4 |
| 685* | 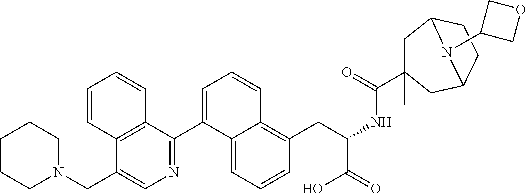 [SMILES: CC1(C(=O)N[C@@H](Cc2cccc3c(-c4ncc(CN5CCCCC5)c5ccccc45)cccc23)C(=O)O)CC2CCC(C1)N2C1COC1] |
| | m/z = 647.2 |
| 686 | 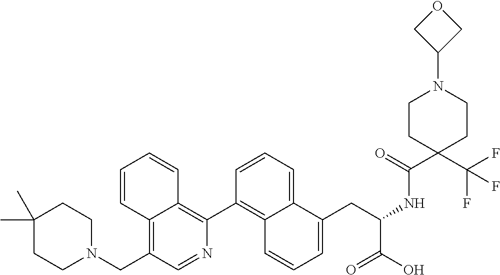 [SMILES: CC1(C)CCN(Cc2cnc(-c3cccc4c(C[C@H](NC(=O)C5(C(F)(F)F)CCN(C6COC6)CC5)C(=O)O)cccc34)c3ccccc23)CC1] |
| | m/z = 703.3 |
| 687 | 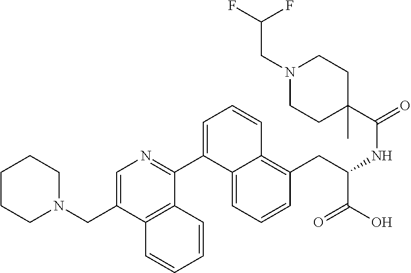 [SMILES: CC1(C(=O)N[C@@H](Cc2cccc3c(-c4ncc(CN5CCCCC5)c5ccccc45)cccc23)C(=O)O)CCN(CC(F)F)CC1] |
| | m/z = 629.3 |
| 688 | 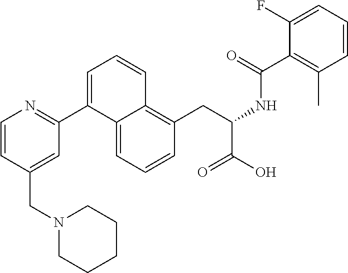 [SMILES: Cc1cccc(F)c1C(=O)N[C@@H](Cc1cccc2c(-c3cc(CN4CCCCC4)ccn3)cccc12)C(=O)O] |
| | m/z = 526.2 |
| 689 | 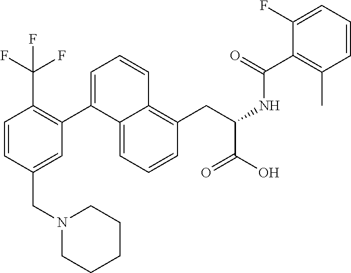 [SMILES: Cc1cccc(F)c1C(=O)N[C@@H](Cc1cccc2c(-c3cc(CN4CCCCC4)ccc3C(F)(F)F)cccc12)C(=O)O] |
| | m/z = 593.3 |
| 690 | 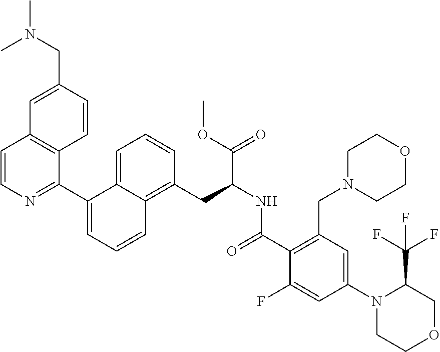 [SMILES: COC(=O)[C@H](Cc1cccc2c(-c3nccc4cc(CN(C)C)ccc34)cccc12)NC(=O)c1c(F)cc(N2CCOC[C@@H]2C(F)(F)F)cc1CN1CCOCC1] |
| | m/z = 788.2 |
| 691 | 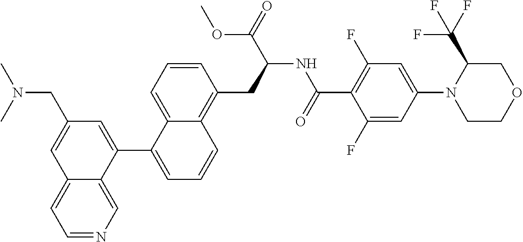 [SMILES: COC(=O)[C@H](Cc1cccc2c(-c3cc(CN(C)C)cc4ccncc34)cccc12)NC(=O)c1c(F)cc(N2CCOC[C@@H]2C(F)(F)F)cc1F] |
| | m/z = 706.8 |
| 692 | 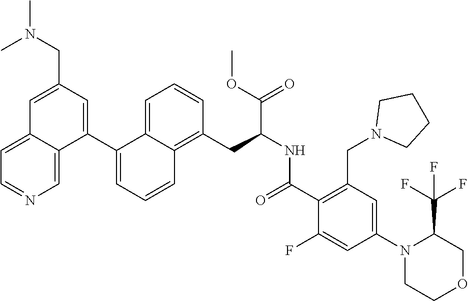 [SMILES: COC(=O)[C@H](Cc1cccc2c(-c3cc(CN(C)C)cc4ccncc34)cccc12)NC(=O)c1c(F)cc(N2CCOC[C@@H]2C(F)(F)F)cc1CN1CCCC1] |
| | m/z = 771.9 |
| 693 | 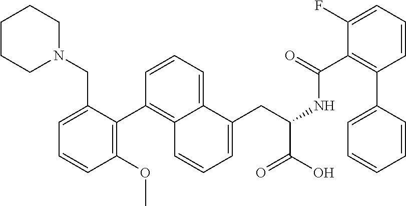 [SMILES: COc1cccc(CN2CCCCC2)c1-c1cccc2c(C[C@H](NC(=O)c3c(F)cccc3-c3ccccc3)C(=O)O)cccc12] |
| | m/z = 617.2 |
| 694 | 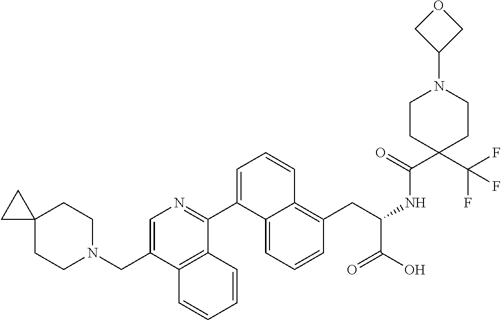 [SMILES: O=C(O)[C@H](Cc1cccc2c(-c3ncc(CN4CCC5(CC4)CC5)c4ccccc34)cccc12)NC(=O)C1(C(F)(F)F)CCN(C2COC2)CC1] |
| | m/z = 701.1 |
| 823 | 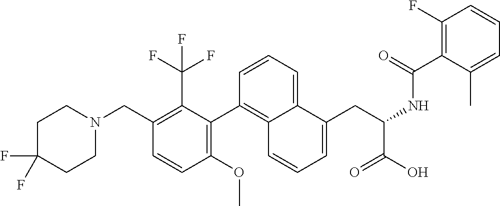 [SMILES: COc1ccc(CN2CCC(F)(F)CC2)c(C(F)(F)F)c1-c1cccc2c(C[C@H](NC(=O)c3c(C)cccc3F)C(=O)O)cccc12] |
| | m/z = 659.1 |
| 850 | 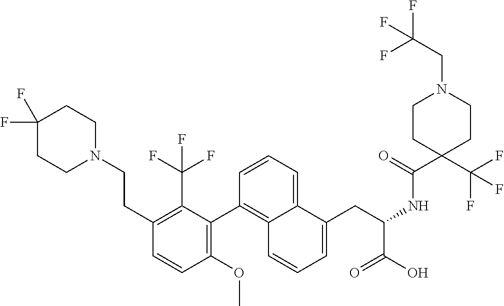 [SMILES: COc1ccc(CCN2CCC(F)(F)CC2)c(C(F)(F)F)c1-c1cccc2c(C[C@H](NC(=O)C3(C(F)(F)F)CCN(CC(F)(F)F)CC3)C(=O)O)cccc12] |
| | m/z = 798.2 |

*Denotes a racemic compound;
**Denotes a single isomer with unknown absolute stereochemistry

### Example 31: General Scheme 1C

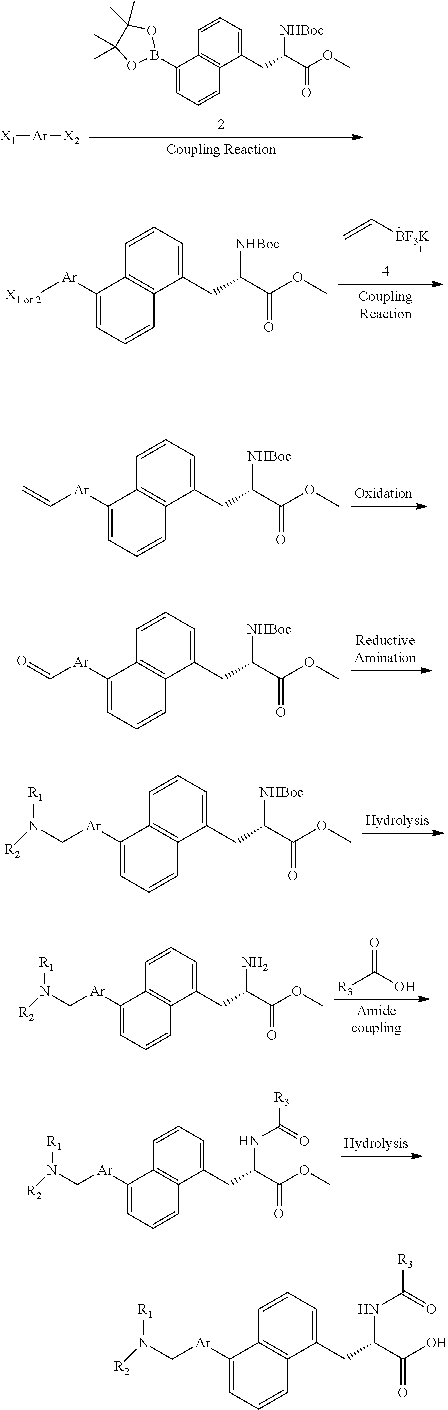

The proceeding compounds were synthesized according to the procedures described in General Scheme 1C using Int1 as an intermediate.

### Synthesis of Compound 695

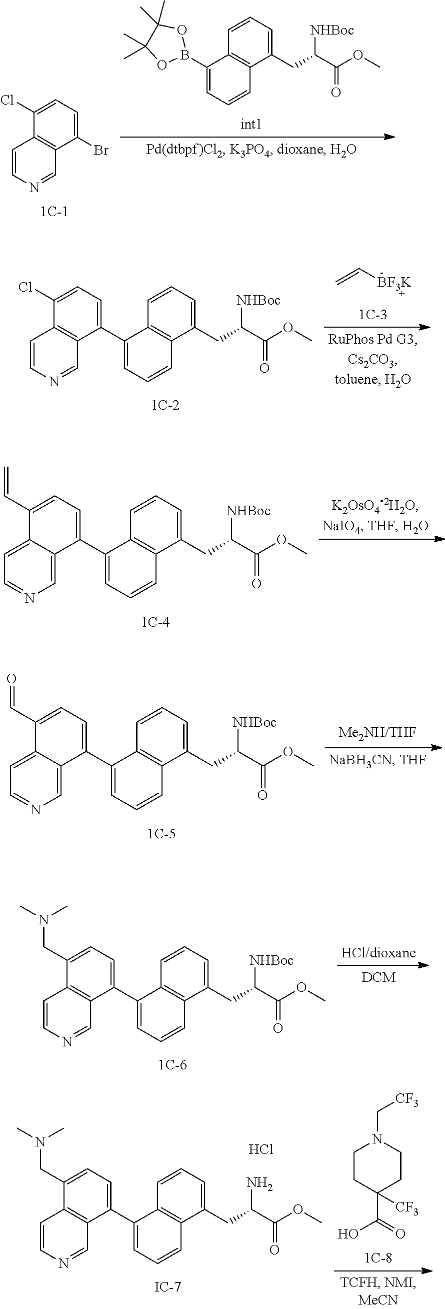

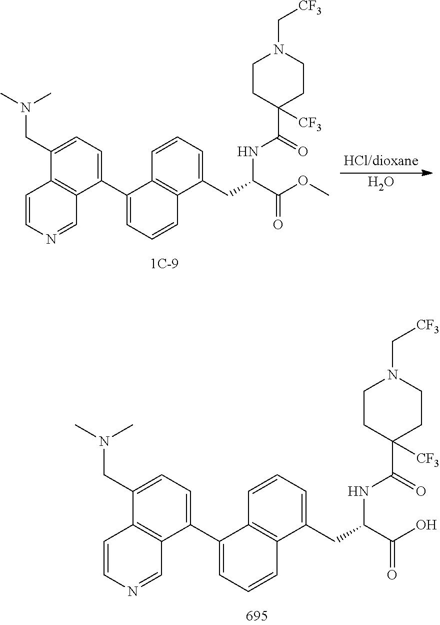

Step 1: A mixture of compound 1C-1 (1.76 g 7.25 mmol, 1.10 eq), compound Int1 (3.00 g, 6.59 mmol, 1.00 eq), Pd(dtbpf)Cl₂ (429 mg, 658 μmol, 0.100 eq), K₃PO₄ (4.20 g, 19.7 mmol, 3.00 eq) in dioxane (30.0 mL) and H₂O (15.0 mL) was degassed and purged with N₂ for 3 times, and then the mixture was stirred at 25° C. for 2 hrs under N₂ atmosphere. The reaction mixture filtered to remove the insoluble material, then the filter cake was washed by ethyl acetate (50.0 ml*3). The filter liquor was diluted with H₂O 30.0 mL and extracted with ethyl acetate 90.0 mL (30.0 mL*3). The combined organic layers were washed with brine 20.0 mL, dried over Na₂SO₄, filtered, and concentrated under reduced pressure to give a residue. The residue was purified by column chromatography (SiO₂, Petroleum ether:Ethyl acetate=3:1 to 1:0). Compound 1C-2 (3.00 g, 6.11 mmol, 92.7% yield) was obtained as yellow oil. LC-MS: (M+H)⁺: 491.2. ¹H NMR: (400 MHz, DMSO-d₆) δ 8.67 (d, J=6.0 Hz, 1H), 8.57 (d, J=12.8 Hz, 1H), 8.30-8.26 (m, 1H), 8.14-8.10 (m, 2H), 7.79-7.73 (m, 1H), 7.69-7.57 (m, 2H), 7.50-7.43 (m, 2H), 7.30 (t, J=8.4 Hz, 1H), 7.12 (d, J=8.8 Hz, 1H), 4.45-4.35 (m, 1H), 3.70-3.63 (m, 4H), 3.41-3.36 (m, 1H), 1.29 (s, 9H).

Step 2: A mixture of compound 1C-2 (3.00 g, 6.11 mmol, 1.00 eq), compound 1C-3 (982 mg, 7.33 mmol, 1.20 eq), RuPhos Pd G3 (511 mg, 611 μmol, 0.100 eq), Cs₂CO₃ (5.97 g, 18.3 mmol, 3.00 eq) in toluene (30.0 mL) and H₂O (15.0 mL) was degassed and purged with N₂ for 3 times, and then the mixture was stirred at 80° C. for 12 hrs under N₂ atmosphere. The reaction mixture filtered to remove the insoluble, then the filter cake was washed by ethyl acetate (50.0 ml*3). The filter liquor was diluted with H₂O 30.0 mL and extracted with ethyl acetate 90.0 mL (30.0 mL*3). The combined organic layers were washed with brine 20.0 mL, dried over Na₂SO₄, filtered, and concentrated under reduced pressure to give a residue. The residue was purified by column chromatography (SiO₂, Petroleum ether:Ethyl acetate=3:1 to 0:1). Compound 1C-4 (2.30 g, 4.77 mmol, 78.0% yield) was obtained as yellow solid. LC-MS: (M+H)⁻: 483.2. ¹H NMR: (400 MHz, DMSO-d₆) δ 8.58-8.53 (m, 2H), 8.29-8.25 (m, 1H), 8.19 (d, J=6.0 Hz, 1H), 8.13 (d, J=7.2 Hz, 1H), 7.79-7.72 (m, 1H), 7.69-7.60 (m, 2H), 7.60-7.56 (m, 1H), 7.50-7.41 (m, 2H), 7.29 (t, J=8.4 Hz, 1H), 7.15-7.12 (m, 1H), 6.10-6.04 (m, 1H), 5.67-5.60 (m, 1H), 4.44-4.38 (m, 1H), 3.70-3.60 (m, 4H), 3.42-3.34 (m, 1H), 1.30 (s, 9H).

Step 3: To a solution of compound 1C-4 (600 mg, 1.24 mmol, 1.00 eq) in THF (5.00 mL) and H₂O (5.00 mL) was added K₂OsO₄·2H₂O (45.8 mg, 124 μmol, 0.100 eq) at 0° C. and stirred for 10 min, then NaIO₄ (797 mg, 3.73 mmol, 206 μL, 3.00 eq) was added at 0° C. The mixture was stirred at 40° C. for 2 hrs. The reaction mixture was quenched by addition Na₂SO₃ 50.0 mL at 0° C., and then diluted with ethyl acetate 45.0 mL and extracted with H₂O 45.0 mL (15.0 mL*3). The combined organic layers were washed with ethyl acetate 45.0 mL (15.0 mL*3), filtered, and concentrated under reduced pressure to give a residue. The residue was purified by Prep-HPLC (column: Phenomenex C18 250*50 mm*10 um; mobile phase: [water (NH₄HCO₃)-ACN]; gradient: 42%-72% B over 10 min). Compound 1C-5 (180 mg, 371 μmol, 29.8% yield) was obtained as yellow solid. LC-MS: (M+H)⁺: 485.2. ¹H NMR: (400 MHz, DMSO-d₆) δ 10.5 (s, 1H), 9.05 (d, J=6.0 Hz, 1H), 8.71-8.57 (m, 3H), 8.33-8.29 (m, 1H), 7.90 (dd, J₁=16.4 Hz, J₂=7.6 Hz, 1H), 7.82-7.75 (m, 1H), 7.64-7.59 (m, 1H), 7.51-7.44 (m, 2H), 7.31 (t, J=8.4 Hz, 1H), 7.11 (d, J=8.4 Hz, 1H), 4.45-4.37 (m, 1H), 3.70-3.62 (m, 4H), 3.42-3.35 (m, 1H), 1.29 (s, 9H).

Step 4: To a solution of compound 1C-5 (150 mg, 309 μmol, 1.00 eq) in THF (1.00 mL) was added Me₂NH (2.00 M, 5.00 mL, 32.3 eq). The mixture was stirred at 25° C. for 12 hrs. Then NaBH CN (58.3 mg, 928 μmol, 3.00 eq) was added at 0° C. The mixture was stirred at 40° C. for 2 hrs. The reaction mixture was extracted with ethyl acetate 45.0 mL (15.0 mL*3). The combined organic layers were washed with brine 10.0 mL, dried over Na₂SO₄, filtered, and concentrated under reduced pressure to give a residue. The residue was purified by Prep-TLC (SiO₂, Petroleum ether:Ethyl acetate=1:1, R_{f}=0.24). Compound 1C-6 (120 mg, 233 μmol, 75.5% yield) was obtained as yellow solid. LC-MS: (M+H)⁺: 514.2

Step 5: To a solution of compound 1C-6 (120 mg, 233 μmol, 1.00 eq) in DCM (2.00 mL) was added HCl/dioxane (4.00 M, 1.50 mL, 25.7 eq) at 0° C. The mixture was stirred at 25° C. for 1 hr. The reaction mixture was concentrated under reduced pressure to give a residue. Compound 1C-7 (90.0 mg, 200 μmol, 85.6% yield, HCl) was obtained as yellow solid. LC-MS: (M+H)⁺: 414.2

Step 6: To a solution of compound 1C-7 (80.0 mg, 177 μmol, 1.00 eq, HCl) and compound 1C-8 (59.5 mg, 213 μmol, 1.20 eq) in MeCN (2.00 mL) was added NMI (51.1 mg, 622 μmol, 49.6 μL, 3.50 eq) and TCFH (59.8 mg, 213 μmol, 1.20 eq). The mixture was stirred at 25° C. for 2 hrs. The reaction mixture was concentrated under reduced pressure to give a residue. The extracted with ethyl acetate 45.0 ml. (15.0 ml.*3). The combined organic layers were washed with brine 10.0 mL, dried over Na₂SO₄, filtered, and concentrated under reduced pressure to give a residue. The residue was purified by Prep-TLC (SiO₂, Dichloromethane:Methanol=10:1, R_{f}=0.65). Compound 1C-9 (110 mg, 163 μmol, 91.7% yield) was obtained as yellow oil. LC-MS: (M+H)⁺: 675.2.

Step 7: To a solution of compound 1C-9 (100 mg, 148 μmol, 1.00 eq) in H₂O (1.00 mL) was added HCl/dioxane (4.00 M, 2.73 mL, 73.6 eq). The mixture was stirred at 60° C. for 2 hrs. The reaction mixture was concentrated under reduced pressure to give a residue. The residue was purified by Prep-HPLC (column: Waters xbridge 150*25 mm 10 um; mobile phase: [water (NH₄HCO₃)-ACN]; gradient: 28%-58% B over 14 min). Compound 695 (12.64 mg, 18.9 μmol, 12.7% yield, 98.8% purity) was obtained as white solid. LC-MS: (M+H)⁺: 661.3. ¹H NMR: (400 MHz, DMSO-d₆) δ 8.68-8.65 (m, 1H), 8.56-8.50 (m, 2H), 8.25 (d, J=6.0 Hz, 1H), 8.04-8.01 (m, 1H), 7.76-7.71 (m, 1H), 7.68 (t, J=7.2 Hz, 1H), 7.52 (d, J=7.2 Hz, 1H), 7.43-7.40 (m, 1H), 7.24-7.17 (m, 1H), 7.14-7.09 (m, 1H), 4.98-4.93 (m, 1H), 4.58 (s, 2H), 3.99-3.84 (m, 1H), 3.58-3.44 (m, 1H), 2.90-2.73 (m, 10H), 2.31-2.07 (m, 4H), 1.85-1.75 (m, 2H).

The compounds set forth in Table 16, were prepared using the synthetic procedures of Example 31, or analogous procedures as provided herein.

TABLE 16
Structure and Spectroscopic data for Compounds 696 to 702

| No. | Structure |
|---|---|
| 696 | 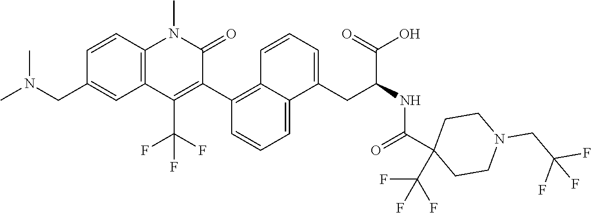 [SMILES: CN(C)Cc1ccc2c(c1)c(C(F)(F)F)c(-c1cccc3c(C[C@H](NC(=O)C4(C(F)(F)F)CCN(CC(F)(F)F)CC4)C(=O)O)cccc13)c(=O)n2C] |
| | m/z = 759.2 |
| 697 | 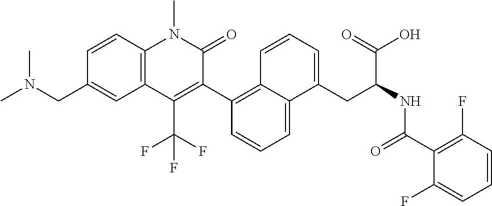 [SMILES: CN(C)Cc1ccc2c(c1)c(C(F)(F)F)c(-c1cccc3c(C[C@H](NC(=O)c4c(F)cccc4F)C(=O)O)cccc13)c(=O)n2C] |
| | m/z = 637.9 |
| 698 | 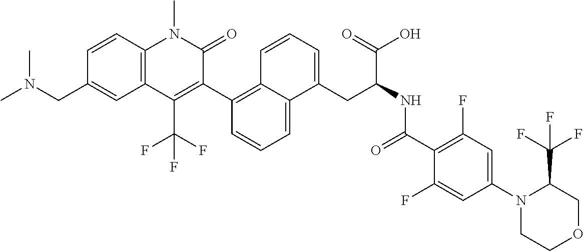 [SMILES: CN(C)Cc1ccc2c(c1)c(C(F)(F)F)c(-c1cccc3c(C[C@H](NC(=O)c4c(F)cc(N5CCOC[C@@H]5C(F)(F)F)cc4F)C(=O)O)cccc13)c(=O)n2C] |
| | m/z = 791.3 |
| 699 | 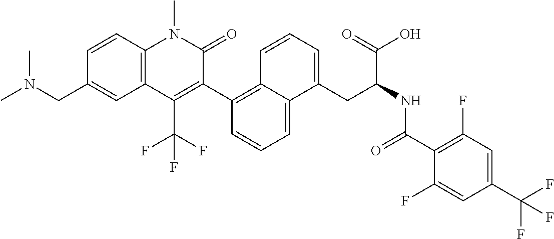 [SMILES: CN(C)Cc1ccc2c(c1)c(C(F)(F)F)c(-c1cccc3c(C[C@H](NC(=O)c4c(F)cc(C(F)(F)F)cc4F)C(=O)O)cccc13)c(=O)n2C] |
| | m/z = 706.4 |
| 700 | 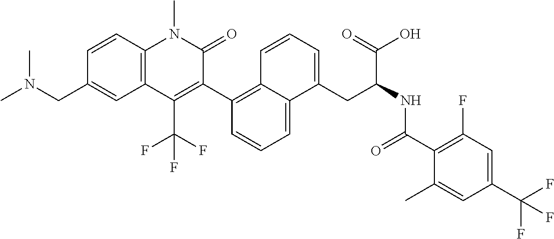 [SMILES: Cc1cc(C(F)(F)F)cc(F)c1C(=O)N[C@@H](Cc1cccc2c(-c3c(C(F)(F)F)c4cc(CN(C)C)ccc4n(C)c3=O)cccc12)C(=O)O] |
| | m/z = 702.3 |
| 701 | 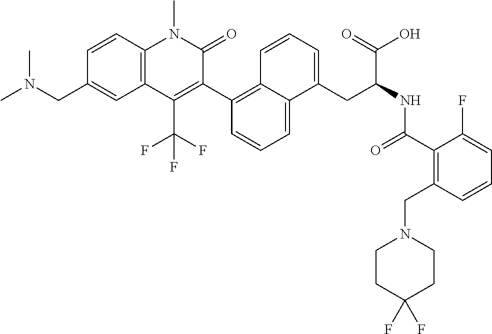 [SMILES: CN(C)Cc1ccc2c(c1)c(C(F)(F)F)c(-c1cccc3c(C[C@H](NC(=O)c4c(F)cccc4CN4CCC(F)(F)CC4)C(=O)O)cccc13)c(=O)n2C] |
| | m/z = 753.3 |
| 702 | 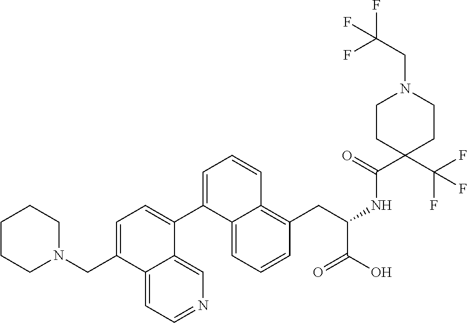 [SMILES: O=C(O)[C@H](Cc1cccc2c(-c3ccc(CN4CCCCC4)c4ccncc34)cccc12)NC(=O)C1(C(F)(F)F)CCN(CC(F)(F)F)CC1] |
| | m/z = 701.3 |
| 704 | 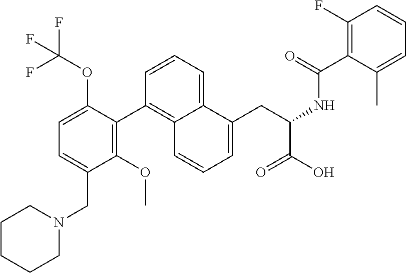 [SMILES: COc1c(CN2CCCCC2)ccc(OC(F)(F)F)c1-c1cccc2c(C[C@H](NC(=O)c3c(C)cccc3F)C(=O)O)cccc12] |
| | m/z = 639.3 |

### Example 32: General Scheme 1D

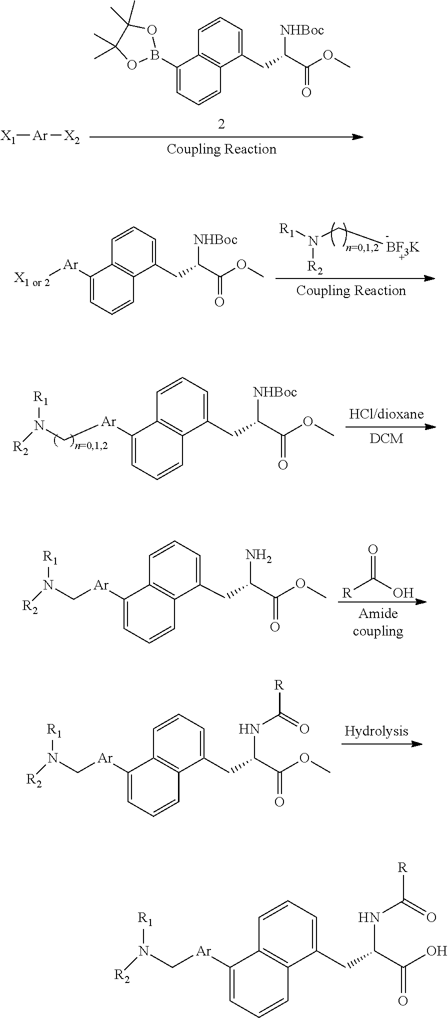

The proceeding compounds were synthesized according to the procedures described in General Scheme 1D using Int1 as an intermediate.

### Synthesis of Compound 703

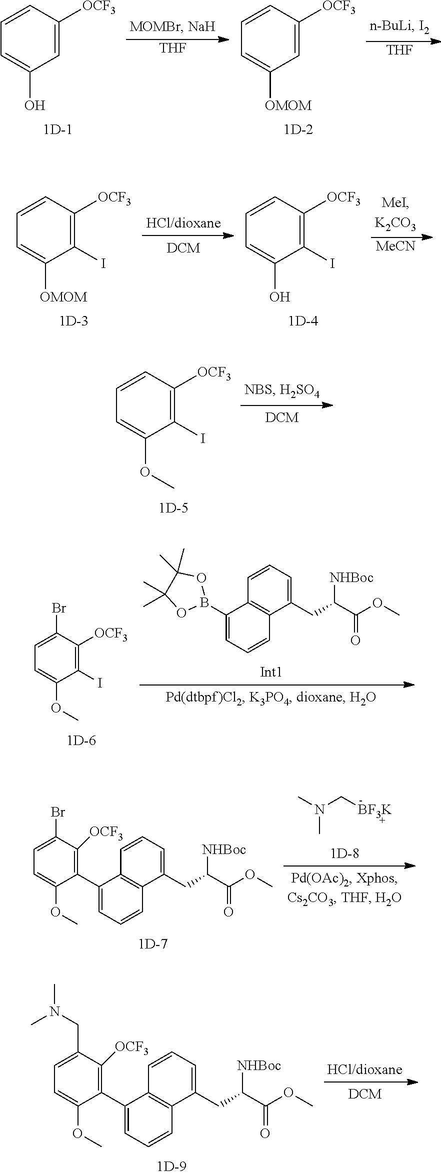

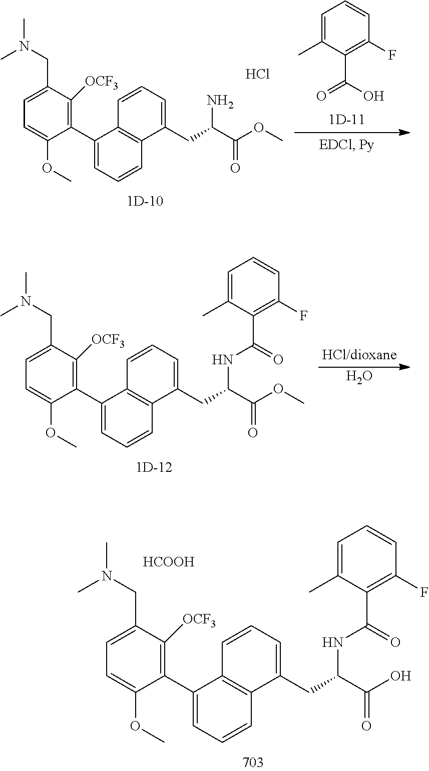

Step 1: To a solution of compound 1D-1 (5.00 g, 28.1 mmol, 1.00 eq) in THF (50.0 mL) was added NaH (1.68 g, 42.1 mmol, 60.0% purity, 1.50 eq) at 0° C. The mixture was stirred at 0° C. for 0.5 hr, then MOMBr (7.02 g, 56.2 mmol, 4.58 mL, 2.00 eq) was added at 0° C., the mixture was warmed to 25° C. and stirred at 25° C. for 2 hrs. The reaction mixture was quenched by addition sat aq. NH₄Cl (20.0 mL) at 0° C., and then diluted with H₂O (20.0 mL) and extracted with ethyl acetate (50.0 mL*3). The combined organic layers were washed with brine (50.0 mL*3), dried over Na₂SO₄, filtered, and concentrated under reduced pressure to give a residue. The residue was purified by column chromatography (SiO₂, Petroleum ether:Ethyl acetate=1:0 to 1:1). Compound 1D-2 (5.32 g, 24.0 mmol, 85.3% yield) was obtained as colorless oil. ¹H NMR: (400 MHz, CDCl₃) δ 7.30 (t, J=8.4 Hz, 1H), 7.01-6.96 (m, 1H), 6.92 (s, 1H), 6.90-6.85 (m, 1H), 5.19 (s, 2H), 3.49 (s, 3H).

Step 2: To a solution of compound 1D-2 (1.00 g, 4.50 mmol, 1.00 eq) in THF (5.00 mL) was added n-BuLi (2.50 M, 3.00 mL, 1.67 eq) at −78° C., the mixture was stirred at −78° C. for 30 min, then a solution of I₂ (1.50 g, 5.91 mmol, 1.19 mL, 1.31 eq) in THF (5.00 mL) was added at −78° C. stirred for 1 hr at −78° C. Then the mixture was warmed to 25° C. and stirred for 1 hr. The reaction mixture was quenched by addition sat. aq. Na₂S₂O₃ (20.0 mL) at 0° C., and then diluted with H₂O (20.0 mL) and extracted with ethyl acetate (30.0 ml.*3). The combined organic layers were washed with brine (30.0 mL*3), dried over Na₂SO₄, filtered, and concentrated under reduced pressure to give a residue. Compound 1D-3 (1.50 g, 4.31 mmol, 95.7% yield) was obtained as yellow oil. ¹H NMR: (400 MHz, CDCl₃) δ 7.31 (t, J=8.4 Hz, 1H), 7.01 (dd, J₁=8.4 Hz, J₂=1.2 Hz, 1H), 6.99-6.93 (m, 1H), 5.28 (s, 2H), 3.53 (s, 3H).

Step 3: To a solution of compound 1D-3 (1.500 g, 4.31 mmol, 1.00 eq) in DCM (10.0 mL) was added HCl/dioxane (4.00 M, 15.0 mL, 13.9 eq), then the mixture was stirred at 25° C. for 1 hr. The reaction mixture concentrated under reduced pressure to give a residue. Compound 1D-4 (1.30 g, 4.28 mmol, 99.2% yield) was obtained as yellow oil. ¹H NMR: (400 MHz, CDCl₃) δ 7.29-7.25 (m, 1H), 6.93 (dd, J₁=8.0 Hz, J₂=0.8 Hz, 1H), 6.89-6.85 (m, 1H), 5.76 (s, 1H)

Step 4: To a solution of compound 1D-4 (1.30 g, 4.28 mmol, 1.00 eq) in MeCN (13.0 mL) was added K₂CO₃ (1.77 g, 12.8 mmol, 3.00 eq) and MeI (1.21 g, 8.55 mmol, 532 μL, 2.00 eq), the mixture was stirred at 50° C. for 12 hrs. The residue was diluted with H₂O (20.0 mL) and extracted with ethyl acetate (20.0 mL*3). The combined organic layers were washed with brine (20.0 mL*3), dried over Na₂SO₄, filtered, and concentrated under reduced pressure to give a residue. The residue was purified by prep-TLC (SiO₂, Petroleum ether:Ethyl acetate-20/1). Compound 1D-5 (1.20 g, 3.77 mmol, 88.2% yield) was obtained as yellow oil. ¹H NMR: (400 MHz, CDCl₃) δ 7.34 (t, J=8.0 Hz, 1H), 6.96-6.90 (m, 1H), 6.76 (dd, J₁=8.4 Hz, J₂=1.2 Hz, 1H), 3.93 (s, 3H).

Step 5: To a solution of compound 1D-5 (500 mg, 1.57 mmol, 1.00 eq) in DCM (20.0 mL) was added NBS (308 mg, 1.73 mmol, 1.10 eq) and H₂SO₄ (771 mg, 7.86 mmol, 419 μL, 5.00 eq) at 0° C., the mixture was stirred at 0° C. for 1 hr. The reaction mixture was quenched by addition sat. aq. NaHCO₃ (50.0 mL) at 0° C., and then diluted with H₂O (20.0 mL) and extracted with DCM (30.0 mL*3). The combined organic layers were washed with brine (30.0 mL*3), dried over Na₂SO₄, filtered and concentrated under reduced pressure to give a residue. The residue was purified by prep-TLC (SiO₂, Petroleum ether:Ethyl acetate=1:0). Compound 1D-6 (0.200 g, 504 μmol, 32.1% yield) was obtained as yellow oil. ¹H NMR: (400 MHz, CDCl₃) δ 7.59 (d, J=9.2 Hz, 1H), 6.68 (d, J=8.8 Hz, 1H), 3.92 (s, 3H).

Step 6: To a solution of compound 1D-6 (180 mg, 454 μmol, 1.00 eq) and compound Int1 (206 mg, 453 μmol, 1.00 eq) in dioxane (5.00 mL) and H₂O (1.00 mL) was added K₃PO₄ (289 mg, 1.36 mmol, 3.00 eq) and Pd(dtbpf)Cl₂ (30.0 mg, 45.4 μmol, 0.100 eq) at 25° C., the mixture was stirred at 25° C. for 1 hr. The residue was diluted with H₂O (30.0 mL) and extracted with ethyl acetate (20.0 mL*3). The combined organic layers were washed with brine (20.0 mL*2), dried over Na₂SO₄, filtered, and concentrated under reduced pressure to give a residue. The residue was purified by prep-TLC (SiO₂, Petroleum ether:Ethyl acetate=3:1). Compound 1D-7 (0.100 g, 134 μmol, 29.5% yield, 80.0% purity) was obtained as yellow oil. LC-MS: (M-99): 498.0.

Step 7: To a solution of compound 1D-7 (90.0 mg, 150 μmol, 1.00 eq) and compound 1D-8 (74.5 mg, 451 μmol, 3.00 eq) in THF (5.00 mL) and H₂O (0.500 mL) was added Cs₂CO₃ (147 mg, 451 μmol, 3.00 eq), XPhos (21.5 mg, 45.1 μmol, 0.300 eq) and Pd(OAc)₂ (3.38 mg, 15.0 μmol, 0.100 eq) at 25° C., the mixture was stirred at 65° C. for 8 hrs. The residue was diluted with H₂O (20.0 mL) and extracted with ethyl acetate (20.0 mL*2). The combined organic layers were washed with brine (20.0 mL*3), dried over Na₂SO₄, filtered, and concentrated under reduced pressure to give a residue. The residue was purified by prep-TLC (SiO₂, DCM:MeOH=10:1). Compound 1D-9 (40.0 mg, 69.4 μmol, 46.1% yield, - purity) was obtained as yellow oil. LC-MS: (M+H)⁺: 577.3.

Step 8: To a solution of compound 1D-9 (40.0 mg, 69.4 μmol, 1.00 eq) in DCM (2.00 mL) was added HCl/dioxane (4.00 M, 0.500 mL, 28.8 eq), the mixture was stirred at 25° C. for 0.5 hr. The mixture was concentrated under reduced pressure to give a residue. Compound 1D-10 (34.0 mg, 66.3 μmol, 95.6% yield, HCl) was obtained as white solid. LC-MS: (M+H)⁺: 477.3

Step 9: To a solution of compound 1D-10 (34.0 mg, 66.3 μmol, 1.00 eq, HCl) and compound 1D-11 (15.3 mg, 99.4 μmol, 1.50 eq) in Py (2.00 mL) was added EDCI (25.4 mg, 133 μmol, 2.00 eq), the mixture was stirred at 25° C. for 1 hr. The residue was diluted with sat. aq. NaHCO₃ (20.0 mL) and extracted with ethyl acetate (20.0 mL*3). The combined organic layers were washed with brine (20.0 mL*3), dried over Na₂SO₄, filtered and concentrated under reduced pressure to give a residue. Compound 1D-12 (40.0 mg, 65.3 μmol, 98.5% yield) was obtained as yellow oil. LC-MS: (M+H)⁺: 613.2

Step 10: To a solution of compound 1D-12 (40.0 mg, 65.3 μmol, 1.00 eq) in H₂O (1.00 mL) was added HCl/dioxane (4.00 M, 5.00 mL, 306 eq), the mixture was stirred at 60° C. for 1 hr. The mixture was concentrated under reduced pressure to give a residue. The residue was purified by prep-HPLC (column: Welch ultimate C18 150*25 mm*7 um; mobile phase: [water (FA)-ACN]; gradient: 12%-42% B over 10 min). Compound 703 (16.14 mg, 25.0 μmol, 38.2% yield, 99.7% purity, FA) was obtained as white solid. LC-MS: (M+H)⁺: 599.3. ¹H NMR: (400 MHz, CD₃CN+D₂O) δ 8.38 (d, J=4.0 Hz, 1H), 8.29-8.24 (m, 1H), 7.73-7.58 (m, 2H), 7.47-7.35 (m, 2H), 7.33-7.15 (m, 4H), 7.02-6.84 (m, 2H), 4.85 (br dd, J₁=9.2 Hz, J₂=5.2 Hz, 1H), 4.20-4.08 (m, 2H), 3.89-3.70 (m, 1H), 3.66 (d, J=3.2 Hz, 3H), 3.46-3.25 (m, 1H), 2.67 (s, 6H), 2.04-1.97 (d, J=20.0 Hz, 3H).

### Example 33: General Scheme 1E

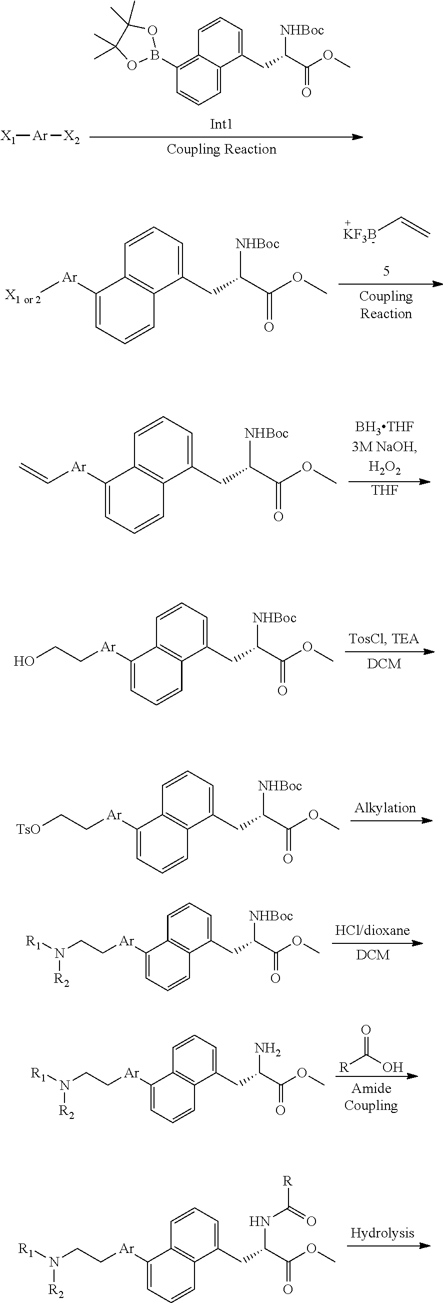

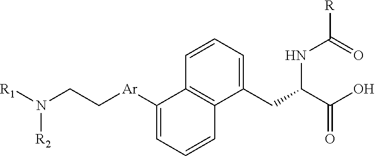

The proceeding compounds were synthesized according to the procedures described in General Scheme 1E using Int1 as an intermediate.

### Synthesis of Compound 705

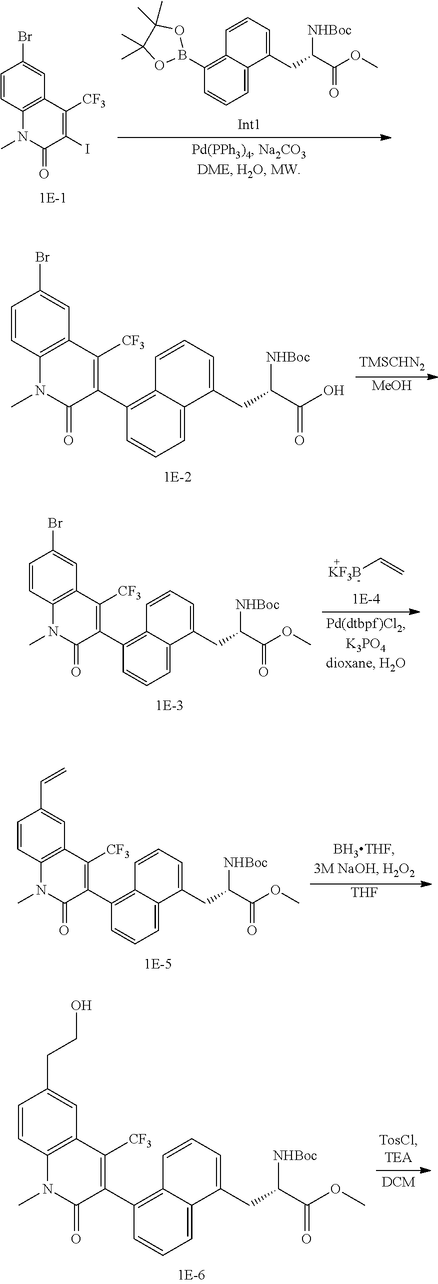

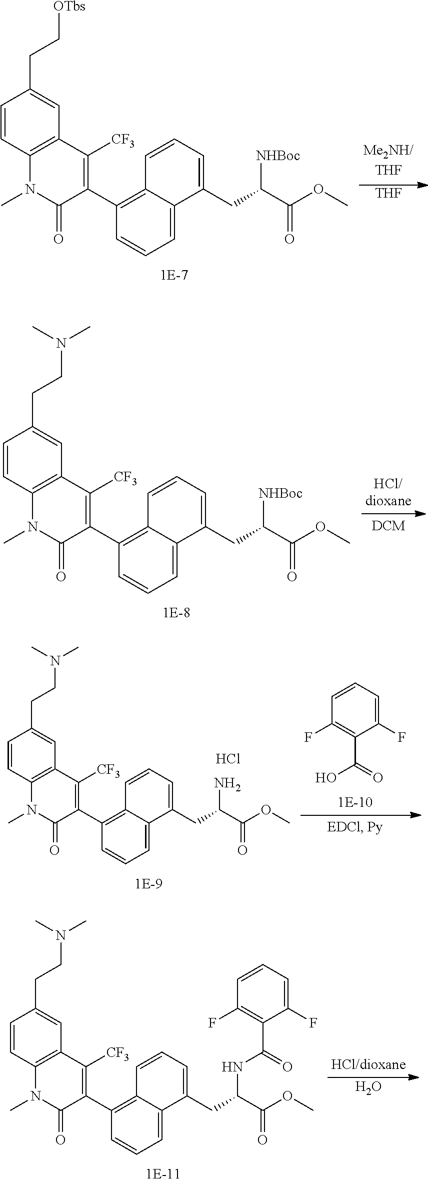

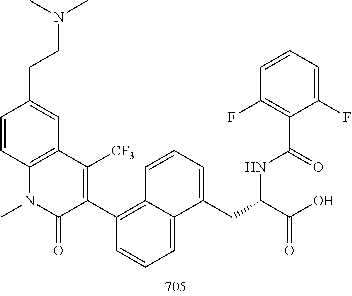 [SMILES: CN(C)CCc1ccc2c(c1)c(C(F)(F)F)c(-c1cccc3c(C[C@H](NC(=O)c4c(F)cccc4F)C(=O)O)cccc13)c(=O)n2C]

Step 1: Compound 1E-1 (0.500 g, 1.16 mmol, 1.00 eq, two batches), compound Int1 (580 mg, 1.27 mmol, 1.10 eq), Na₂CO₃ (368 mg, 3.47 mmol, 3.00 eq) and Pd(PPh₃)₄ (134 mg, 116 μmol, 0.100 eq) were taken up into a microwave tube in DME (10.0 mL) and H₂O (3.00 mL). The sealed tube was heated at 130° C. for 1 hr under microwave. The reaction mixture was concentrated under reduced pressure to remove DME. The residue was diluted with H₂O (40.0 mL) and adjusted pH to 3 with HCl (1.00 M). The aqueous phase was extracted with ethyl acetate (20.0 mL*3). The combined organic layers were dried over Na₂SO₄, filtered, and concentrated under reduced pressure to give a residue. The residue was purified by column chromatography (SiO₂, Petroleum ether:Ethyl acetate=100:1 to 0:1). Compound 1E-2 (1.20 g, crude) was obtained as yellow solid. LC-MS: (M-55)⁺: 564.8

Step 2: To a solution of compound 1E-2 (1.20 g, 1.94 mmol, 1.00 eq) in MeOH (10.0 mL) was added TMSCHN₂ (2.00 M, 2.91 mL, 3.00 eq) at 0° C., then the mixture was stirred at 25° C. for 2 hrs. The reaction mixture was quenched by addition sat. aq. NH₄Cl (50.0 mL) at 0° C., and then diluted with H₂O (20.0 mL) and extracted with ethyl acetate (30.0 mL*2). The combined organic layers were washed with brine (30.0 mL*2), dried over Na₂SO₄, filtered, and concentrated under reduced pressure to give a residue. Compound 1E-3 (1.20 g, 1.89 mmol, 97.8% yield) was obtained as yellow solid.

Step 3: To a solution of compound 1E-3 (1.20 g, 1.89 mmol, 1.00 eq) and compound 1E-4 (2.54 g, 18.9 mmol, 10.0 eq) in dioxane (20.0 mL) and H₂O (5.00 mL) was added K₃PO₄ (1.21 g, 5.68 mmol, 3.00 eq), Pd(dtbpf)Cl₂ (123 mg, 189 μmol, 0.100 eq), the mixture was stirred at 20° C. for 12 hrs. The residue was diluted with H₂O (20.0 mL) and extracted with ethyl acetate (15.0 mL*2). The combined organic layers were washed with brine (15.0 mL*2), dried over Na 2SO₄, filtered, and concentrated under reduced pressure to give a residue. The residue was purified by column chromatography (SiO₂, Petroleum ether:Ethyl acetate=100:1 to 0:1). Compound 1E-5 (0.500 g, 821 μmol, 43.4% yield, 95.4% purity) was obtained as yellow solid. LC-MS: (M-55)⁻: 525.1. ¹H NMR: (400 MHz, CDCl₃) δ 8.15 (d, J=8.4 Hz, 1H), 7.98 (s, 1H), 7.82 (d, J=8.4 Hz, 1H), 7.60 (t, J=7.2 Hz, 1H), 7.51 (d, J=9.2 Hz, 1H), 7.44 (d, J=8.4 Hz, 1H), 7.36-7.28 (m, 3H), 6.84 (dd, J₁=17.6 Hz, J₂=10.8 Hz, 1H), 5.85 (d, J=17.6 Hz, 1H), 5.38 (d, J=10.8 Hz, 1H), 5.08 (d, J=7.6 Hz, 1H), 4.75 (d, J=6.8 Hz, 1H), 3.85-3.79 (m, 3H), 3.70-3.50 (m, 5H), 1.43 (d, J=11.6 Hz, 9H).

Step 4: To a solution of compound 1E-5 (0.500 g, 822 μmol, 95.4% purity, 1.00 eq) in THF (10.0 mL) was added BH₃THF (1.00 M, 2.48 mL, 3.02 eq) at 0° C. And the mixture was warmed to 25° C. and stirred at 25° C. for 5 hrs. Then NaOH (3.00 M, 826 μL, 3.02 eq) and H₂O₂ (290 mg, 2.56 mmol, 246 μL, 30.0% purity, 3.11 eq) were added at 0° C. Then the mixture was warmed to 0° C. and stirred at 0° C. for 3 hrs. The reaction mixture was quenched by sat. aq. Na₂SO₃ (10.0 mL), sat. aq. NH₄Cl (10.0 mL) at 0° C., and H₂O (10.0 mL). The aqueous phase was extracted with ethyl acetate (20.0 mL*2). The combined organic layers were washed with brine (20.0 mL*2), dried over Na₂SO₄, filtered, and concentrated under reduced pressure to give a residue. The residue was purified by Prep-TLC (SiO₂, DCM:MeOH=10:1). Compound 1E-6 (0.230 g, 375 μmol, 45.6% yield, 97.5% purity) was obtained as white solid. LC-MS: (M-55)⁺: 543.1. ¹H NMR: (400 MHz, CDCl₃) δ 8.15 (d, J=8.4 Hz, 1H), 8.01-7.88 (m, 1H), 7.64-7.57 (m, 2H), 7.56-7.48 (m, 1H), 7.43 (d, J=8.0 Hz, 1H), 7.35-7.28 (m, 4H), 5.08 (d, J=7.2 Hz, 1H), 4.78-4.74 (m, 1H), 3.97 (t, J=6.4 Hz, 2H), 3.83-3.78 (m, 4H), 3.58 (s, 3H), 3.03 (t, J=6.0 Hz, 2H), 1.43 (d, J=11.2 Hz, 9H).

Step 5: To a solution of compound 1E-6 (200 mg, 325 μmol, 97.5% purity, 1.00 eq) in DCM (5.00 mL) was added TEA (98.9 mg, 977 μmol, 136 μL, 3.00 eq) and TosCl (124 mg, 651 μmol, 2.00 eq), the mixture was stirred at 20° C. for 1 hr. The residue was diluted with H₂O (10.0 mL) and extracted with DCM (10.0 mL*3). The combined organic layers were washed with brine (10.0 mL*3), dried over Na₂SO₄, filtered and concentrated under reduced pressure to give a residue. The residue was purified by Prep-TLC (SiO₂, Petroleum ether:Ethyl acetate=1:3). Compound 1E-7 (130 mg, 171 μmol, 52.5% yield, 99.1% purity) was obtained as yellow oil. LC-MS: (M-55)⁺: 697.2. ¹H NMR: (400 MHz, DMSO-d₆) δ 8.13 (t, J=8.0 Hz, 1H), 7.67-7.61 (m, 6H), 7.49-7.41 (m, 2H), 7.37-7.32 (m, 5H), 4.37-4.30 (m, 3H), 3.70 (s, 3H), 3.63-3.56 (m, 4H), 3.38-3.33 (m, 1H), 3.05 (t, J=6.0 Hz, 2H), 2.36 (s, 3H), 1.33 (d, J=5.2 Hz, 9H).

Step 6: To a solution of compound 1E-7 (130 mg, 171 μmol, 99.1% purity, 1.00 eq) in THF (1.00 mL) was Me₂NH (1.00 M, 10.0 mL, 58.4 eq, in THF), then the mixture was stirred at 25° C. for 24 hrs. The mixture was concentrated under reduced pressure to give a residue. Compound 1E-8 (107 mg, 171 μmol, 99.9% yield) was obtained as yellow solid. LC-MS: (M+H)⁺: 626.3

Step 7: To a solution of compound 1E-8 (107 mg, 171 μmol, 1.00 eq) in DCM (2.00 mL) was added HCl/dioxane (4.00 M, 2.00 mL, 46.8 eq), then the mixture was stirred at 25° C. for 2 hrs. The mixture was concentrated under reduced pressure to give a residue. Compound 1E-9 (96.0 mg, 171 μmol, 99.8% yield, HCl) was obtained as yellow solid. LC-MS: (M+H)⁺: 526.0

Step 8: To a solution of compound 1E-9 (46.0 mg, 81.8 μmol, 1.00 eq, HCl) and compound 1E-10 (25.9 mg, 163 μmol, 2.00 eq) in Py (2.00 mL) was added EDCI (31.4 mg, 163 μmol, 2.00 eq), the mixture was stirred at 25° C. for 1 hr. The reaction mixture was concentrated under reduced pressure to remove Py. The residue was diluted with sat. aq. NaHCO₃ (15.0 mL) and extracted with ethyl acetate (15.0 mL*3). The combined organic layers were dried over Na₂SO₄, filtered, and concentrated under reduced pressure to give a residue. Compound 1E-11 (0.050 g, 75.1 μmol, 91.7% yield) was obtained as yellow solid. LC-MS: (M+H)⁺: 666.2

Step 9: To a solution of compound 1E-11 (45.0 mg, 67.6 μmol, 1.00 eq) in H₂O (2.00 mL) was added HCl/dioxane (4.00 M, 6.00 mL, 355 eq), then mixture was stirred at 60° C. for 2 hrs. The mixture was concentrated under reduced pressure to give a residue. The residue was purified by Prep-HPLC (column: Welch Xtimate C18 150*25 mm*5 um; mobile phase: [water (NH₄HCO₃)-ACN]; B %: 15%-45%, 2 min). Compound 705 (16.86 mg, 24.7 μmol, 36.6% yield, 95.7% purity) was obtained as white solid. LC-MS: (M+H)⁺: 652.3. ¹H NMR: (400 MHz, DMSO-d₆) δ 9.12 (d, J=6.8 Hz, 1H), 8.23 (d, J=8.4 Hz, 1H), 7.75 (s, 1H), 7.71-7.67 (m, 2H), 7.66-7.61 (m, 1H), 7.53-7.42 (m, 2H), 7.40-7.37 (m, 1H), 7.35-7.30 (m, 2H), 7.12 (t, J=7.6 Hz, 2H), 4.75-4.71 (m, 1H), 3.78-3.69 (m, 4H), 3.43-3.39 (m, 1H), 2.89 (t, J=6.8 Hz, 2H), 2.59-2.56 (m, 2H), 2.25 (s, 6H).

The compounds set forth in Table 18, were prepared using the synthetic procedures of Example 33, or analogous procedures as provided herein.

TABLE 18
Structure and Spectroscopic data for Compounds 706 to 715

| No. | Structure |
|---|---|
| 706 | 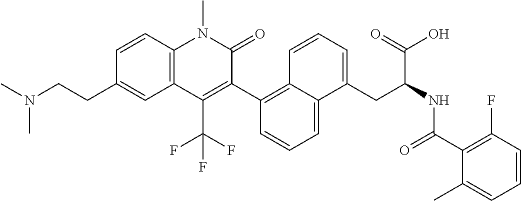 [SMILES: Cc1cccc(F)c1C(=O)N[C@@H](Cc1cccc2c(-c3c(C(F)(F)F)c4cc(CCN(C)C)ccc4n(C)c3=O)cccc12)C(=O)O] |
| | m/z = 648.1 |
| 707 | 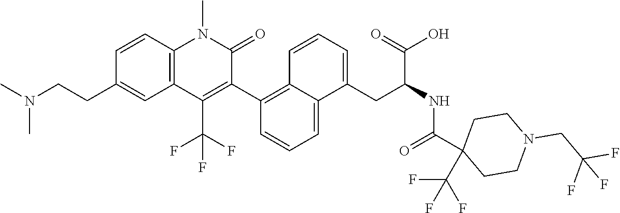 [SMILES: CN(C)CCc1ccc2c(c1)c(C(F)(F)F)c(-c1cccc3c(C[C@H](NC(=O)C4(C(F)(F)F)CCN(CC(F)(F)F)CC4)C(=O)O)cccc13)c(=O)n2C] |
| | m/z = 773.2 |
| 708 | 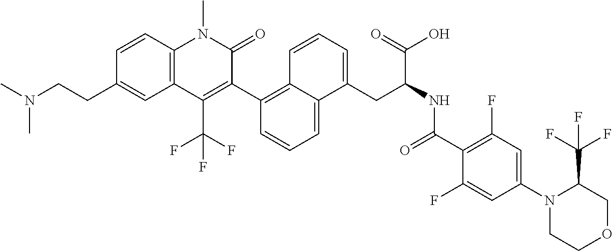 [SMILES: CN(C)CCc1ccc2c(c1)c(C(F)(F)F)c(-c1cccc3c(C[C@H](NC(=O)c4c(F)cc(N5CCOC[C@@H]5C(F)(F)F)cc4F)C(=O)O)cccc13)c(=O)n2C] |
| | m/z = 805.3 |
| 709 | 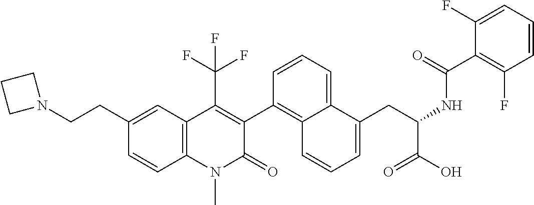 [SMILES: Cn1c(=O)c(-c2cccc3c(C[C@H](NC(=O)c4c(F)cccc4F)C(=O)O)cccc23)c(C(F)(F)F)c2cc(CCN3CCC3)ccc21] |
| | m/z = 664.5 |
| 710 | 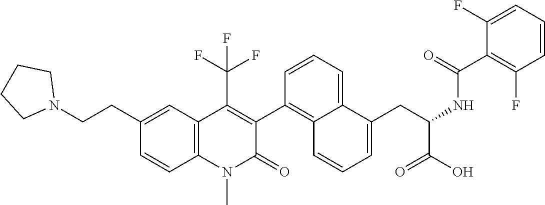 [SMILES: Cn1c(=O)c(-c2cccc3c(C[C@H](NC(=O)c4c(F)cccc4F)C(=O)O)cccc23)c(C(F)(F)F)c2cc(CCN3CCCC3)ccc21] |
| | m/z = 678.5 |
| 711 | 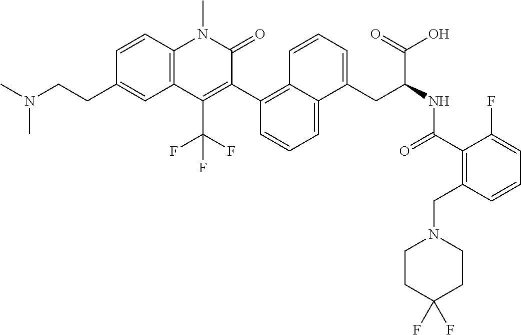 [SMILES: CN(C)CCc1ccc2c(c1)c(C(F)(F)F)c(-c1cccc3c(C[C@H](NC(=O)c4c(F)cccc4CN4CCC(F)(F)CC4)C(=O)O)cccc13)c(=O)n2C] |
| | m/z = 767.4 |
| 712 | 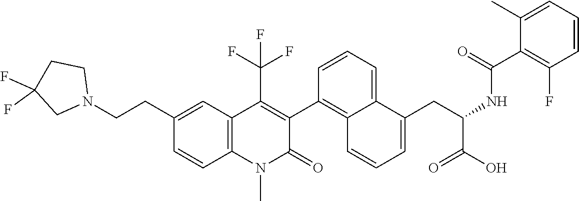 [SMILES: Cc1cccc(F)c1C(=O)N[C@@H](Cc1cccc2c(-c3c(C(F)(F)F)c4cc(CCN5CCC(F)(F)C5)ccc4n(C)c3=O)cccc12)C(=O)O] |
| | m/z = 710.3 |
| 713 | 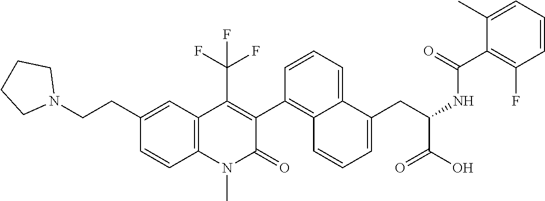 [SMILES: Cc1cccc(F)c1C(=O)N[C@@H](Cc1cccc2c(-c3c(C(F)(F)F)c4cc(CCN5CCCC5)ccc4n(C)c3=O)cccc12)C(=O)O] |
| | m/z = 674.2 |
| 714 | 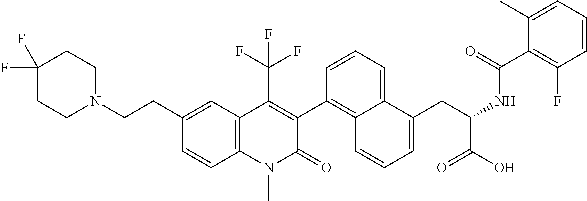 [SMILES: Cc1cccc(F)c1C(=O)N[C@@H](Cc1cccc2c(-c3c(C(F)(F)F)c4cc(CCN5CCC(F)(F)CC5)ccc4n(C)c3=O)cccc12)C(=O)O] |
| | m/z = 724.5 |
| 715 | 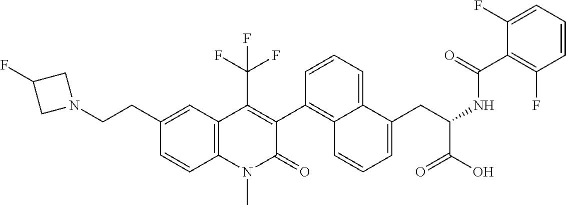 [SMILES: Cn1c(=O)c(-c2cccc3c(C[C@H](NC(=O)c4c(F)cccc4F)C(=O)O)cccc23)c(C(F)(F)F)c2cc(CCN3CC(F)C3)ccc21] |
| | m/z = 682.5 |

### Example 34: General Scheme 1F

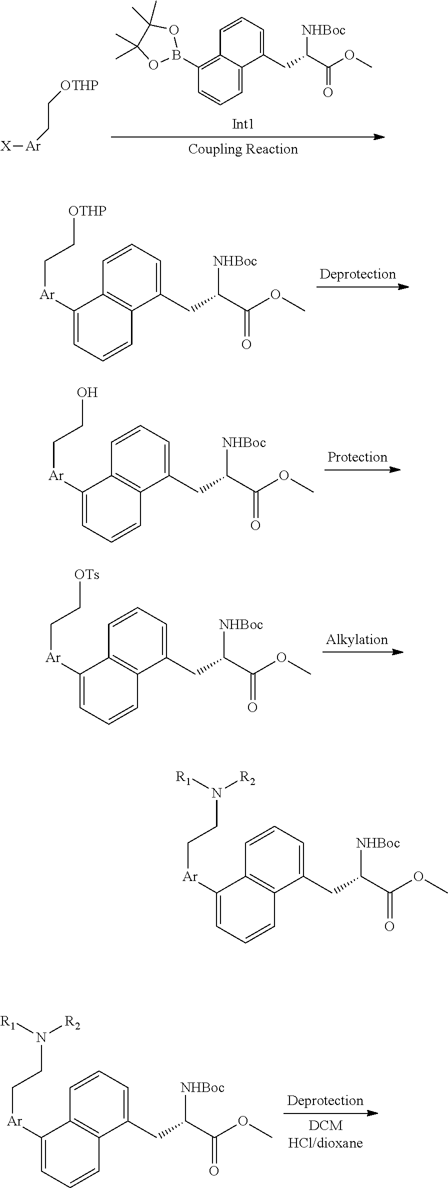

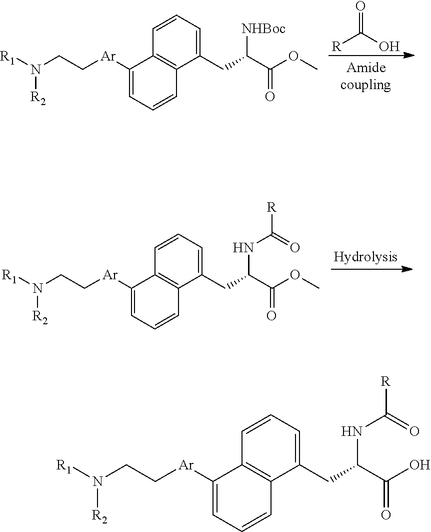

The proceeding compounds were synthesized according to the procedures described in General Scheme 1F using Int1 as an intermediate.

### Synthesis of Compound 716

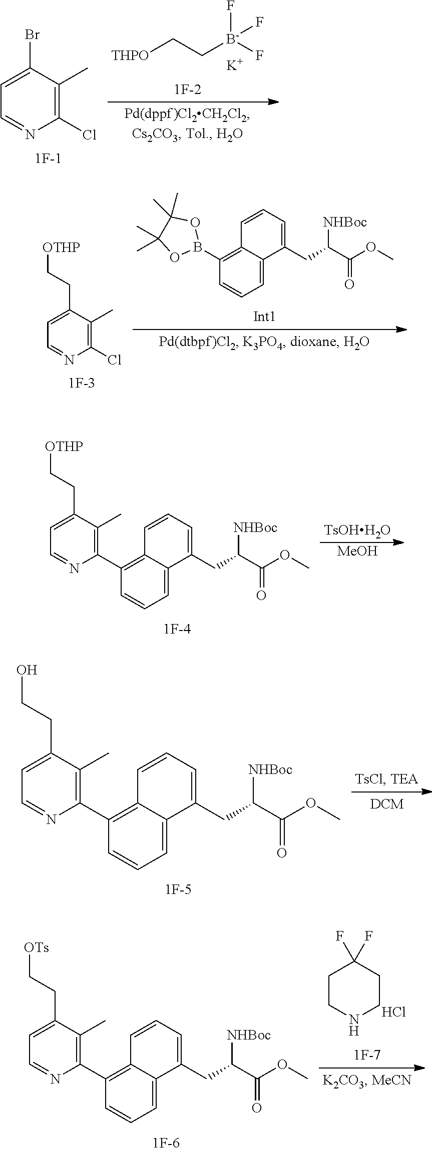

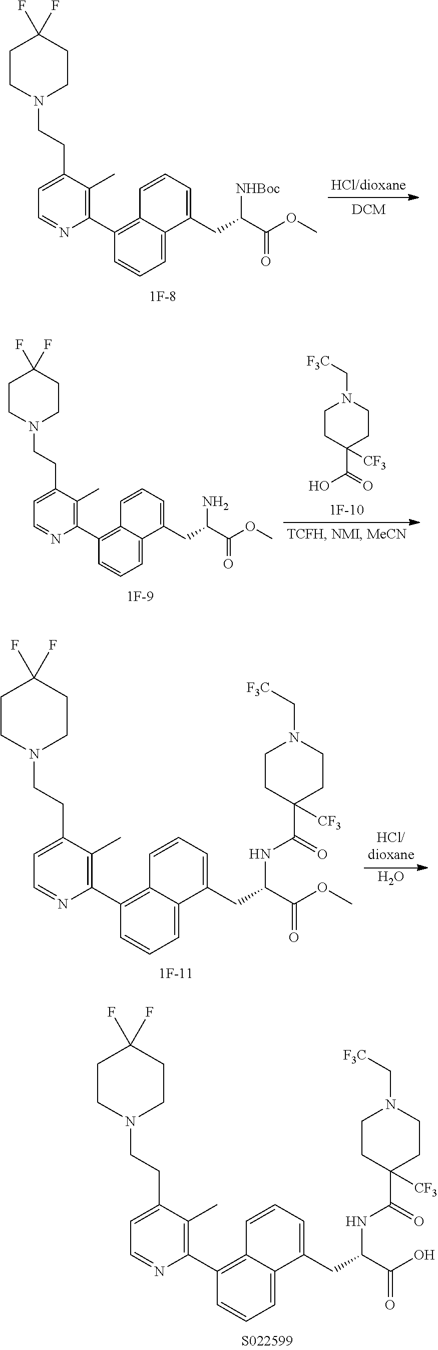

Step 1: A mixture of compound 1F-1 (500 mg, 2.42 mmol, 1.00 eq), compound 1F-2 (1.14 g, 4.84 mmol, 2.00 eq, K), Pd(dppf)Cl₂·CH₂Cl₂ (395 mg, 484 μmol, 0.200 eq), Cs₂CO₃ (2.37 g, 7.27 mmol, 3.00 eq) in Tol. (8.00 mL) and H₂O (2.00 mL) was degassed and purged with N₂ for 3 times, and then the mixture was stirred at 90° C. for 5 hrs under N₂ atmosphere. Water 50 mL was added into reaction mixture. The mixture was diluted with ethyl acetate 20.0 mL and extracted with ethyl acetate (50.0 mL*3). The combined organic layers were washed with brine (30.0 mL*4), dried over Na₂SO₄ filtered and concentrated under reduced pressure to give a residue. The residue was purified by column chromatography (SiO₂, Petroleum ether:Ethyl acetate=15:1 to 10:1). Compound 1F-3 (450 mg, 1.76 mmol, 72.6% yield) was obtained as colorless oil. LC-MS: (M+H)⁺: 256.4.

Step 2: A mixture of compound 1F-3 (395 mg, 1.54 mmol, 1.00 eq), compound Int1 (773 mg, 1.70 mmol, 1.10 eq), Pd(dtbpf)Cl₂ (201 mg, 308 μmol, 0.200 eq), K₃PO₄ (983 mg, 4.63 mmol, 3.00 eq) in dioxane (5.00 mL) and H₂O (1.00 mL) was degassed and purged with N₂ for 3 times, and then the mixture was stirred at 25° C. for 5 hrs under N₂ atmosphere. The reaction mixture was quenched by water 50.0 mL. The mixture was diluted with ethyl acetate 50.0 mL and extracted with ethyl acetate (50.0 mL*3). The combined organic layers were washed with brine (50.0 mL*4), dried over Na₂SO₄ filtered and concentrated under reduced pressure to give a residue. The residue was purified by Prep-TLC (SiO₂, Ethyl acetate:Petroleum ether=2:1). Compound 1F-4 (0.600 g, 1.09 mmol, 70.8% yield) was obtained as a brown solid. LC-MS: (M+H)⁺: 549.6

Step 3: To a solution of compound 1F-4 (500 mg, 911 μmol, 1.00 eq) in MeOH (5.00 mL) was added TsOH·H₂O (156 mg, 911 μmol, 1.00 eq). The mixture was stirred at 25° C. for 2 hrs. Water was added into reaction mixture. The mixture was diluted with ethyl acetate 100 mL, and extracted with ethyl acetate (100 mL*3). The combined organic layers were washed with brine (100 mL*4), dried over Na₂SO₄ filtered and concentrated under reduced pressure to give a residue. Compound 1F-5 (400 mg, 861 μmol, 94.4% yield) was obtained as brown oil.

Step 4: To a solution of compound 1F-5 (350 mg, 753 μmol, 1.00 eq) in DCM (2.00 mL) was added TsCl (287 mg, 1.51 mmol, 2.00 eq) and TEA (228 mg, 2.26 mmol, 314 μL, 3.00 eq). The mixture was stirred at 25° C. for 4 hrs. The reaction mixture was addition water 100 mL, diluted with ethyl acetate 100 mL and extracted with ethyl acetate (100 mL*3). The combined organic layers were washed with brine (100 ml.*4), dried over Na₂SO₄ filtered and concentrated under reduced pressure to give a residue. The residue was purified by Prep-TLC (SiO₂, Petroleum ether:Ethyl acetate=1:1). Compound 1F-6 (450 mg, 727 μmol, 96.5% yield) was obtained as brown solid. LC-MS: (M+H)⁺: 619.4

Step 5: To a solution of compound 1F-6 (200 mg, 323 μmol, 1.00 eq) in MeCN (5.00 mL) was added K₂CO₃ (134 mg, 969 μmol, 3.00 eq) and compound 1F-7 (101 mg, 646 μmol, 2.00 eq, HCl). The mixture was stirred at 70° C. for 12 hrs. Water 100 mL was added into reaction mixture. The mixture was diluted with ethyl acetate 100 mL and extracted with ethyl acetate (100 mL*3). The combined organic layers were washed with brine (100 mL*4), dried over Na₂SO₄ filtered and concentrated under reduced pressure to give a residue. The residue was purified by Prep-TLC (SiO₂, Petroleum ether:Ethyl acetate=0:1). Compound 1F-8 (90.0 mg, 158 μmol, 49.0% yield) was obtained as yellow solid. LC-MS: (M+H)⁺: 568.3

Step 6: To a solution of compound 1F-8 (90.0 mg, 158 μmol, 1.00 eq) in DCM (1.00 mL) was added HCl/dioxane (4.00 M, 2.00 mL, 50.4 eq). The mixture was stirred at 25° C. for 1 hr. The reaction mixture was concentrated under reduced pressure to give a residue. Compound 1F-9 (80.0 mg, crude, HCl) was obtained as a white solid.

Step 7: To a solution of compound 1F-9 (80.0 mg, 158 μmol, 1.00 eq, HCl) in MeCN (3.00 mL) was added TCFH (133 mg, 476 μmol, 3.00 eq), compound 1F-10 (44.3 mg, 158 μmol, 1.00 eq) and NMI (65.1 mg, 793 μmol, 63.2 μL, 5.00 eq). The mixture was stirred at 25° C. for 1 hr. Water 10.0 mL was added into reaction mixture. The resulting mixture was then diluted with ethyl acetate 10.0 mL and extracted with ethyl acetate (20.0 mL*3). The combined organic layers were washed with brine (20.0 mL*4), dried over Na₂SO₄ filtered and concentrated under reduced pressure to give a residue. The residue was purified by Prep-TLC (SiO₂, Petroleum ether:Ethyl acetate=0:1).

Compound 1F-11 (80.0 mg, 109 μmol, 69.1% yield) was obtained as a white solid. LC-MS: (M+H)⁻: 729.1. ¹H NMR: (400 MHz, DMSO-d₆) δ 8.55 (br dd, J₁=7.6 Hz, J₂=4.0 Hz, 1H), 8.39 (d, J=4.8 Hz, 1H), 8.18 (dd, J₁=13.6 Hz, J₂=8.8 Hz, 1H), 7.69-7.65 (m, 1H), 7.42-7.37 (m, 2H), 7.33-7.28 (m, 2H), 7.12 (d, J=8.4 Hz, 1H), 4.89-4.78 (m, 1H), 3.84-3.74 (m, 1H), 3.69 (d, J=6.4 Hz, 3H), 3.58-3.46 (m, 2H), 3.01-2.84 (m, 5H), 2.67-2.65 (m, 3H), 2.61 (br s, 5H), 2.27-2.19 (m, 2H), 1.97-1.89 (m, 7H), 1.62-1.53 (m, 2H)

Step 8: To a solution of compound 1F-11 (70.0 mg, 96.0 μmol, 1.00 eq) in H₂O (2.00 mL) was added HCl/dioxane (4.00 M, 1.40 mL, 58.3 eq). The mixture was stirred at 60° C. for 1 hr. The reaction mixture was concentrated under reduced pressure to give a residue. The residue was purified by Prep-HPLC (column: Welch ultimate C18 150*25 mm*7 um; mobile phase: [water (FA)-ACN]; gradient: 8%-38% B over 10 min) to give resuide, then further purified by Prep-HPLC (column: Waters xbridge 150*25 mm 10 um; mobile phase: [water (NH₄HCO₃)-ACN]; gradient: 26%-56% B over 14 min). Compound 716 (20.06 mg, 26.6 μmol, 27.7% yield, 95.0% purity) was obtained as a white solid. LC-MS: (M+H)⁺: 715.2. ¹H NMR: (400 MHz, DMSO-d₆) δ 8.39 (d, J=4.8 Hz, 1H), 8.32-8.31 (m, 1H), 8.26-8.20 (m, 1H), 7.68-7.63 (m, 1H), 7.43-7.35 (m, 2H), 7.32-7.26 (m, 2H), 7.10 (d, J=8.4 Hz, 1H), 4.77-4.64 (m, 1H), 3.84-3.72 (m, 1H), 3.52-3.49 (m, 1H), 2.99-2.82 (m, 4H), 2.68-2.65 (m, 3H), 2.60 (br s, 5H), 2.25-2.16 (m, 2H), 2.11-2.04 (m, 1H), 2.01-1.92 (m, 7H), 1.87-1.69 (m, 1H), 1.64-1.52 (m, 2H)

The compounds set forth in Table 19, were prepared using the synthetic procedures of Example 34, or analogous procedures as provided herein.

TABLE 19
Structure and Spectroscopic data for Compounds 717 and 718

| No. | Structure |
|---|---|
| 717 | 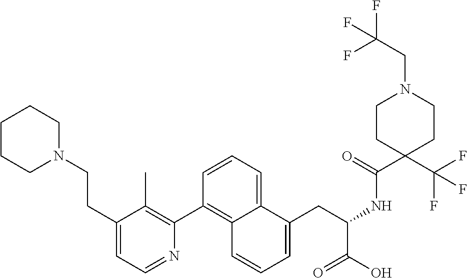 [SMILES: Cc1c(CCN2CCCCC2)ccnc1-c1cccc2c(C[C@H](NC(=O)C3(C(F)(F)F)CCN(CC(F)(F)F)CC3)C(=O)O)cccc12] |
| | m/z = 679.3 |
| 718 | 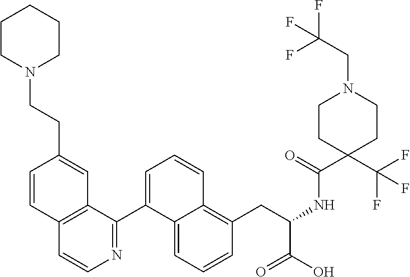 [SMILES: O=C(O)[C@H](Cc1cccc2c(-c3nccc4ccc(CCN5CCCCC5)cc34)cccc12)NC(=O)C1(C(F)(F)F)CCN(CC(F)(F)F)CC1] |
| | m/z = 715.3 |

### Example 35: Synthesis of Compound 719

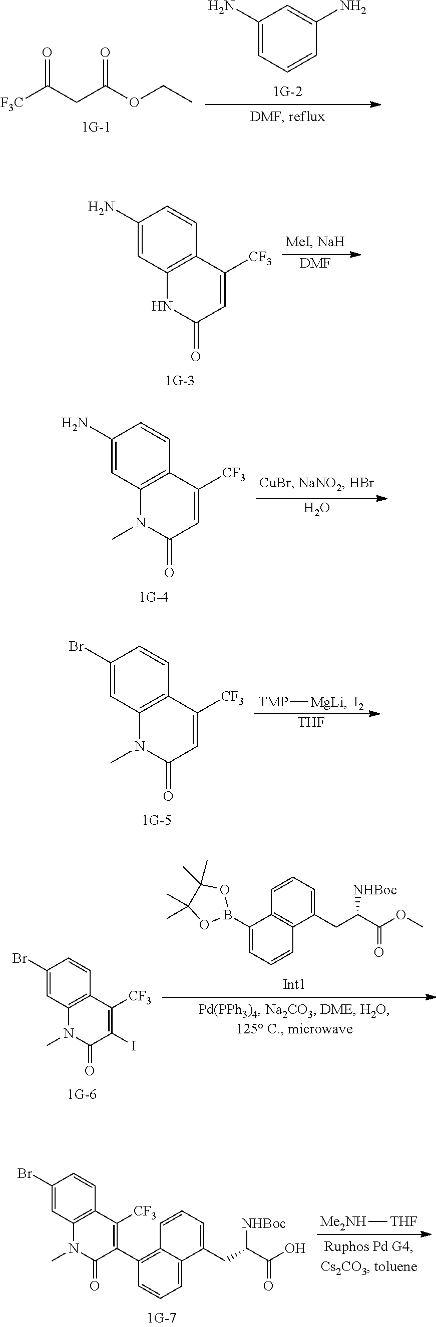

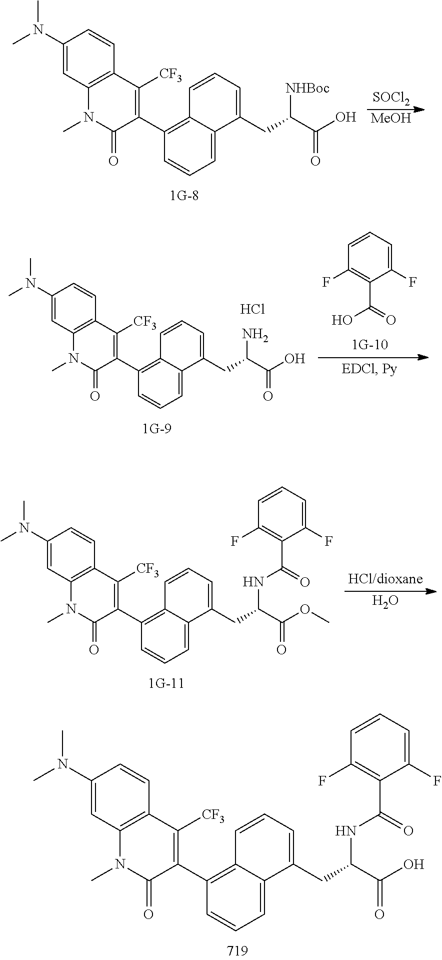

Step 1: To a solution of compound 1G-1 (50.0 g, 271 mmol, 39.6 mL, 1.00 eq), compound 1G-2 (29.3 g, 271 mmol, 1.00 eq) in DMF (400 mL). The mixture was stirred at 140° C. for 12 hrs. The mixture was added water (2.00 L) and precipitate formed. The mixture was filtered to get the filter cake. The filter cake was triturated with ethyl acetate (500 mL) for 2 hrs. Compound 1G-3 (25.0 g, crude) was obtained as a white solid. LC-MS: (M+H)⁺: 229.0. ¹H NMR: (400 MHz, DMSO-d₆). δ 11.81 (s, 1H), 7.36-7.33 (m, 1H), 6.56 (dd, J₁=8.8 Hz, J₂=2.4 Hz, 1H), 6.46 (d, J=2.4 Hz, 1H), 6.42 (s, 1H), 6.14 (s, 2H).

Step 2: A mixture of compound 1G-3 (10.0 g, 43.8 mmol, 1.00 eq) in DMF (150 mL) was added NaH (2.10 g, 52.6 mmol, 60% purity, 1.20 eq) at 0° C. and the mixture was stirred at 0° C. for 30 min. Mel (7.46 g, 52.6 mmol, 3.27 mL, 1.20 eq) was added at 0° C. and the mixture was stirred at 25° C. for 1.5 hrs. The reaction mixture was quenched by addition saturated solution NH₄Cl 200 mL at 0° C. and stirred for 0.5 hr, and then extracted with ethyl acetate (200 mL*3). The combined organic layers were dried over Na₂SO₄, filtered, and concentrated under reduced pressure to give a residue. The residue was purified by Prep-HPLC (column: Phenomenex luna C 18 (250*70 mm, 10 μm); mobile phase: [water (NH₄HCO₃)-ACN]; B %: 24%-44%, 20 min). Compound 1G-4 (4.50 g, 18.6 mmol, 42.4% yield) was obtained as a yellow solid. LC-MS: (M+H)⁺: 243.0. ¹H NMR: (400 MHz, DMSO-d₆) δ 7.43 (d, J=8.4 Hz, 1H), 6.67-6.61 (m, 2H), 6.55 (s, 1H), 6.27 (s, 2H), 3.52 (s, 3H)

Step 3: A solution of compound 1G-4 (2.70 g, 11.1 mmol, 1.00 eq) in HBr (29.8 g, 176 mmol, 20.0 mL, 48% purity, 15.8 eq) and H₂O (10.0 mL) at −5° C. for 30 min, and then NaNO₂ (923 mg, 13.4 mmol, 1.20 eq) in H₂O (15.0 mL) was added at 0° C. and stirred for 1 hr. After CuBr (2.40 g, 16.7 mmol, 509 μL, 1.50 eq) was added, the mixture was stirred at 25° C. for 1 hr. The reaction mixture was quenched by addition NH₄Cl saturated solution 50.0 mL. Sodium sulfite 50 mL was added into aqueous layer. The aqueous layer was extracted with ethyl acetate (100 mL*3). The combined organic layers were washed with brine (80.0 mL*2), dried over, filtered, and concentrated under reduced pressure to give a residue. Compound 1G-5 (3.60 g, crude) was obtained as a yellow solid. LC-MS: (M+H)⁺: 307.8. ¹H NMR: (400 MHz, DMSO-d₆) δ 7.89 (d, J=1.6 Hz, 1H), 7.67 (dd, J₁=8.8 Hz, J₂=1.6 Hz, 1H), 7.57 (dd, J₁=8.8 Hz, J₂=1.6 Hz, 1H), 7.13 (s, 1H), 3.64 (s, 3H)

Step 4: To a stirred solution of compound 1G-5 (3.60 g, 11.7 mmol, 1.00 eq) in THF (40.0 mL) at −78° C. was added TMP-MgLi (1 M, 30.6 mL, 2.60 eq). The reaction mixture was allowed to stir at −78° C. for 30 min, then I₂ (7.46 g, 29.4 mmol, 5.92 mL, 2.50 eq) in THF (15.0 mL) was added dropwise. The reaction was stirred at −78° C. for an additional 1 hr under N₂ atmosphere. The reaction mixture was quenched by addition sat. aq. NH₄Cl 100 mL and sat. aq. Na₂SO₃ 80.0 mL at 25° C. and extracted with ethyl acetate (100 mL*3). The combined organic layers were washed with brine (100 mL*2), dried over, filtered, and concentrated under reduced pressure to give a residue. The residue was purified by Prep-HPLC (column: Phenomenex luna C 18 250*50 mm*10 um; mobile phase: [water (NH₄HCO₃)-ACN]; B %: 35%-65%, 20 min). Compound 1G-6 (2.10 g, 4.86 mmol, 41.3% yield) was obtained as a yellow solid. LC-MS: (M+H)⁺: 433.6. ¹H NMR: (400 MHz, DMSO-d₆) δ 7.88 (s, 1H), 7.77 (dd, J₁=8.8 Hz, J₂=2.0 Hz, 1H), 7.54 (d, J=8.8 Hz, 1H), 3.74 (s, 3H)

Step 5: Compound 1G-6 (1.00 g, 2.31 mmol, 1.00 eq), compound Int1 (1.05 g, 2.31 mmol, 1.00 eq) and Pd(PPh₃)₄ (267 mg, 231 μmol, 0.100 eq), Na₂CO₃ (491 mg, 4.63 mmol, 2.00 eq) were taken up into a microwave tube in DME (15.0 mL), H₂O (5.00 mL). The sealed tube was heated at 125° C. for 30 min under microwave. The reaction mixture was adjusted to pH 6 with 1M HCl and partitioned between H₂O 20.0 mL and ethyl acetate (20.0 mL*3). The organic phase was separated, washed with brine 20.0 mL, filtered, and concentrated under reduced pressure to give a residue. The residue was purified by Prep-HPLC (column: Welch Xtimate C 18 250*70 mm #10 um; mobile phase: [water (NH₄HCO₃)-ACN]; B %: 25%-55%, 20 min). Compound 1G-7 (650 mg, 1.05 mmol, 45.3% yield) was obtained as a yellow solid. LC-MS: (M+18): 637.8. ¹H NMR: (400 MHz, DMSO-d₆) δ 8.25 (t, J=7.2 Hz, 1H), 7.97 (d, J=1.6 Hz, 1H), 7.87-7.80 (m, 1H), 7.66-7.63 (m, 1H), 7.62-7.53 (m, 2H), 7.42 (t, J=8.0 Hz, 1H), 7.37-7.33 (m, 1H), 7.30 (d, J=6.8 Hz, 2H), 4.14 (dd, J_{1=4.0} Hz, J₂=2.0 Hz, 1H), 3.70 (d, J=4.0 Hz, 4H), 3.64 (br s, 1H), 1.31 (d, J=10.4 Hz, 9H)

Step 6: A mixture of compound 1G-7 (250 mg, 403 μmol, 1.00 eq), Cs₂CO₃ (263 mg, 807 μmol, 2.00 eq), Me₂NH·THF (2 M, 403 μL, 2.00 eq), Ruphos Pd G4 (34.3 mg, 40.3 μmol, 0.100 eq) in toluene (4.00 mL) was degassed and purged with N₂ for 3 times, and then the mixture was stirred at 90° C. for 5 hrs under N₂ atmosphere. The reaction mixture was concentrated under reduced pressure to remove toluene and adjust pH to 5 with 1M HCl, diluted with H₂O 10 mL and extracted with dichloromethane (10.0 mL*3). The combined organic layers were washed with brine 15.0 mL, dried over Na₂SO₄, filtered, and concentrated under reduced pressure to give a residue. The residue was purified by Prep-TLC (SiO₂, Dichloromethane:Methanol=10:1). Compound 1G-8 (165 mg, 282 μmol, 70.0% yield) was obtained as a yellow solid. LC-MS: (M+H)⁺: 584.3. ¹H NMR: (400 MHz, DMSO-d₆) δ 8.21 (t, J=7.2 Hz, 1H), 7.77-7.65 (m, 1H), 7.54 (br d, J=6.0 Hz, 1H), 7.39 (d, J=7.2 Hz, 1H), 7.33-7.27 (m, 2H), 7.25 (d, J=6.8 Hz, 1H), 6.92 (dd, J₁=9.2 Hz, J₂=2.0 Hz, 1H), 6.60 (s, 2H), 4.14 (d, J=6.0 Hz, 1H), 3.79-3.69 (m, 1H), 3.67 (d, J=4.0 Hz, 3H), 3.64-3.57 (m, 1H), 3.13 (s, 6H), 1.32 (d, J=9.6 Hz, 9H).

Step 7: A mixture of compound 1G-8 (150 mg, 257 μmol, 1.00 eq) in MeOH (2.00 mL) was added SOCl₂ (984 mg, 8.27 mmol, 0.600 mL, 32.1 eq) slowly, and then the mixture was stirred at 60° C. for 1 hr. The reaction mixture was concentrated under reduced pressure to give a residue. Compound 1G-9 (150 mg, crude, HCl) was obtained as a yellow solid. LC-MS: (M+H): 498.1.

Step 8: To a solution of compound 1G-9 (80.0 mg, 149 μmol, 1.00 eq, HCl), compound 1G-10 (23.6 mg, 149 μmol, 1.00 eq) in Py (1.00 mL) was added EDCI (57.4 mg, 299 μmol, 2.00 eq). The mixture was stirred at 25° C. for 1 hr. The reaction mixture was concentrated under reduced pressure to give a residue. Compound 1G-11 (60.0 mg, crude) was obtained as a yellow solid. LC-MS: (M+H)⁺: 638.1

Step 9: To a solution of compound 1G-11 (60.0 mg, 94.1 μmol, 1.00 eq) in H₂O (0.500 mL) was added HCl/dioxane (4 M, 1.00 mL, 42.5 eq). The mixture was stirred at 60° C. for 1 hr. The reaction mixture was concentrated under reduced pressure to give a residue. The residue was purified by Prep-HPLC (column: Welch Xtimate C 18 150*25 mm*5 um; mobile phase: [water (NH₃H₂O)-ACN]; B %: 23%-53%, 8 min) and (column: Phenomenex luna C 18 150*25 mm*10 um; mobile phase: [water (FA)-ACN]; B %: 50%-80%, 10 min). Compound 719 (16.21 mg, 25.4 μmol, 27.0% yield, 97.9% purity) was obtained as a white solid. LC-MS: (M+H)⁺: 623.9. ¹H NMR: (400 MHz, DMSO-d₆) δ 9.19 (br s, 1H), 8.19 (d, J=8.4 Hz, 1H), 7.70 (td, J₁=9.2 Hz, J₂=2.8 Hz, 1H), 7.66-7.58 (m, 1H), 7.54-7.46 (m, 1H), 7.46-7.41 (m, 1H), 7.39-7.28 (m, 3H), 7.16-7.09 (m, 2H), 6.92 (dd, J₁=9.4 Hz, J₂=1.6 Hz 1H), 6.60 (s, 1H), 4.77-4.72 (m, 1H), 3.79-3.70 (m, 1H), 3.67 (d, J=3.6 Hz, 3H), 3.49-3.42 (m, 1H), 3.14 (s, 6H)

### Example 36: Synthesis of Compound 720

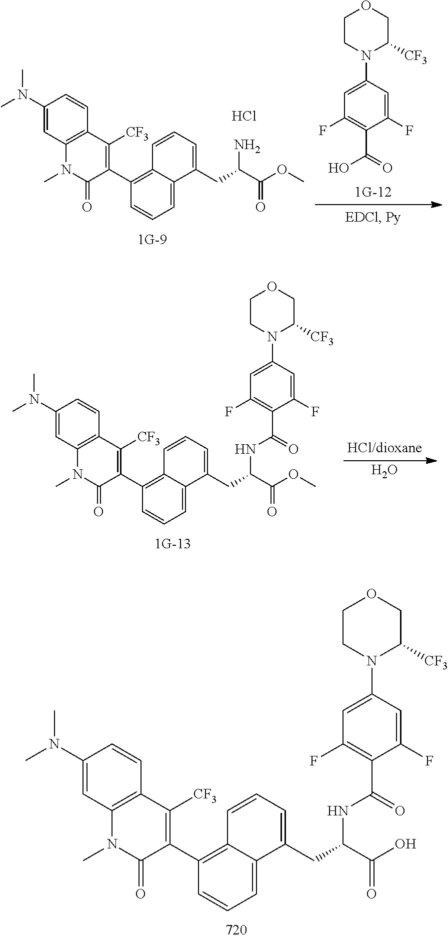

Step 1: To a solution of compound 1G-9 (60.0 mg, 112 μmol, 1.00 eq, HCl), compound 1G-12 (34.9 mg, 112 μmol, 1.00 eq) in Py (1.00 mL) was added EDCI (43.1 mg, 224 μmol, 2.00 eq). The mixture was stirred at 25° C. for 1 hrs. The reaction mixture was concentrated under reduced pressure to give a residue. Compound 1G-13 (50.0 mg, crude) was obtained as a yellow solid. LC-MS: (M+H)⁺: 791.3

Step 2: To a solution of compound 1G-13 (50.0 mg, 63.2 μmol, 1.00 eq) in H₂O (0.500 mL) was added HCl/dioxane (4 M, 714 μL, 45.2 eq). The mixture was stirred at 60° C. for 1 hr. The reaction mixture was concentrated under reduced pressure to give a residue. The residue was purified by Prep-HPLC (column: Welch Xtimate C 18 150*25 mm*5 um; mobile phase: [water (NH₃H₂O)-ACN]; B %: 25%-55%, 8 min) and (column: Phenomenex luna C 18 150*25 mm*10 um; mobile phase: [water (FA)-ACN]; B %: 51%-81%, 10 min). Compound 720 (23.95 mg, 29.7 μmol, 47.1% yield, 96.6% purity) was obtained as a white solid. LC-MS: (M+H)⁺: 776.8. ¹H NMR: (400 MHz, DMSO-d₆) δ 8.83 (s, 1H), 8.21-8.18 (m, 1H), 7.77-7.65 (m, 1H), 7.64-7.57 (m, 1H), 7.43 (t, J=6.0 Hz, 1H), 7.38-7.26 (m, 3H), 6.92 (dd, J₁=9.6 Hz, J₂=2.0 Hz, 1H), 6.82-6.73 (m, 2H), 6.60 (s, 1H), 4.97-4.84 (m, 1H), 4.68-4.65 (m, 1H), 4.16 (d, J=12.4 Hz, 1H), 3.95 (dd, J_{1=11.2} Hz, J₂=3.6 Hz, 1H), 3.74 (d, J=13.6 Hz, 2H), 3.67 (d, J=2.8 Hz, 3H), 3.60-3.52 (m, 2H), 3.44-3.42 (m, 1H), 3.25-3.21 (m, 1H), 3.13 (s, 6H)

### Example 37: General Scheme for the Synthesis of Common Intermediates 2

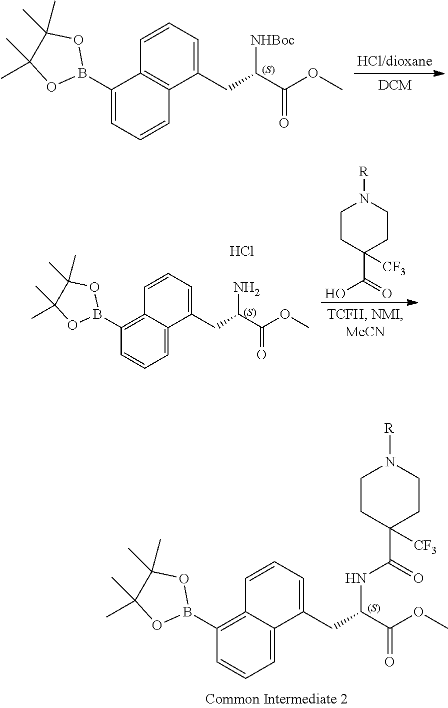

In some embodiments, R of common intermediate 2, is an R group as exemplified in Examples 39 to 48. In some embodiments, R of common intermediate 2 is an R groups as exemplified in compounds 721 to 994.

### Example 38: Synthesis of Intermediate 2 (Int2)

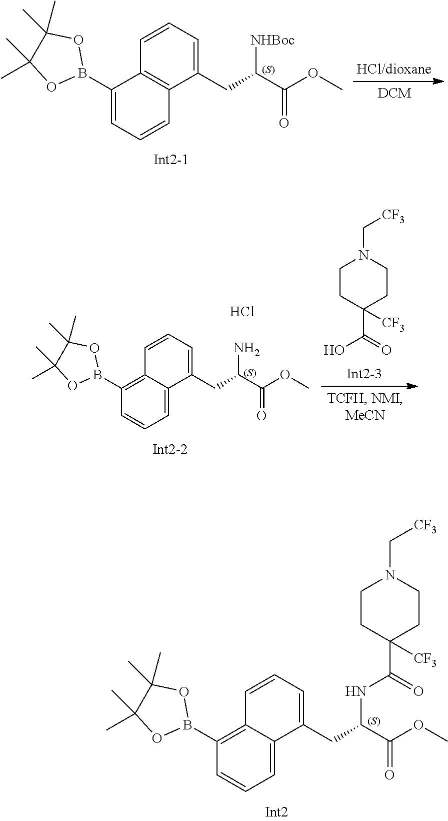

Step 1: To a solution of compound Int2-1 (3.00 g, 6.59 mmol, 1.00 eq) in DCM (30.0 mL) was added HCl/dioxane (4.00 M, 3.00 mL, 1.82 eq), the mixture was stirred at 20° C. for 2 hrs. The reaction mixture was concentrated under reduced pressure to give a residue. Compound Int2-2 (2.50 g, crude, HCl) was obtained as light-yellow oil. LC-MS: (M+H)⁺: 355.9. ¹H NMR: (400 MHz, CDCl₃) δ 9.02 (br s, 3H), 8.72 (d, J=8.4 Hz, 1H), 8.27 (d, J=8.6 Hz, 1H), 8.05 (d, J=6.4 Hz, 1H), 7.53-7.41 (m, 3H), 4.57-4.40 (m, 1H), 4.11-4.01 (m, 1H), 3.84-3.77 (m, 1H), 3.35 (s, 3H), 1.41 (s, 12H).

Step 2: To a solution of compound Int2-2 (2.30 g, 5.87 mmol, 1.00 eq, HCl) and compound Int2-3 (1.64 g, 5.87 mmol, 1.00 eq) in ACN (30.0 mL) was added TCFH (4.94 g, 17.6 mmol, 3.00 eq) and NMI (2.89 g, 35.2 mmol, 2.81 mL, 6.00 eq), the mixture was stirred at 20° C. for 3 hrs. The reaction mixture was concentrated under reduced pressure to remove ACN, diluted with water 50.0 mL and extracted with ethyl acetate 210 mL (70.0 mL*3). The combined organic layers were washed with brine 50.0 mL (50.0 mL*1), dried over Na₂SO₄, filtered, and concentrated under reduced pressure to give a residue. The residue was purified by column chromatography (SiO₂, Petroleum ether:Ethyl acetate=1:1 to 5:1). Compound Int2 (3.27 g, 5.24 mmol, 89.2% yield, 98.7% purity) was obtained as light-yellow oil. LC-MS: (M+H)⁺: 617.2. ¹H NMR: (400 MHz, CDCl₃) δ 8.72 (d, J=8.8 Hz, 1H), 8.21 (d, J=8.4 Hz, 1H), 8.15-8.07 (m, 1H), 7.62-7.50 (m, 1H), 7.48-7.42 (m, 1H), 7.29 (d, J=7.2 Hz, 1H), 6.25 (d, J=7.6 Hz, 1H), 5.10-4.99 (m, 1H), 3.79-3.42 (m, 6H), 2.84-2.67 (m, 3H), 2.84-2.67 (d, J=11.2 Hz, 1H), 2.19 (t, J=10.8 Hz, 1H), 1.93-1.72 (m, 4H), 1.43 (s, 9H).

### Example 40: General Scheme 2A

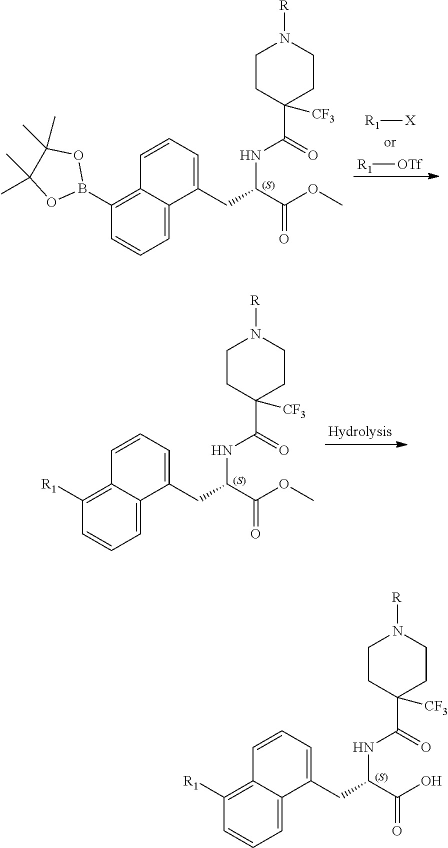

The proceeding compounds were synthesized according to the procedures described in General Scheme 2A using Common Intermediate 2 or Int2 as an intermediate. Compounds 757 to 760 were reduced using Pd/C and H₂ prior to the hydrolysis step.

### Synthesis of Compound 721

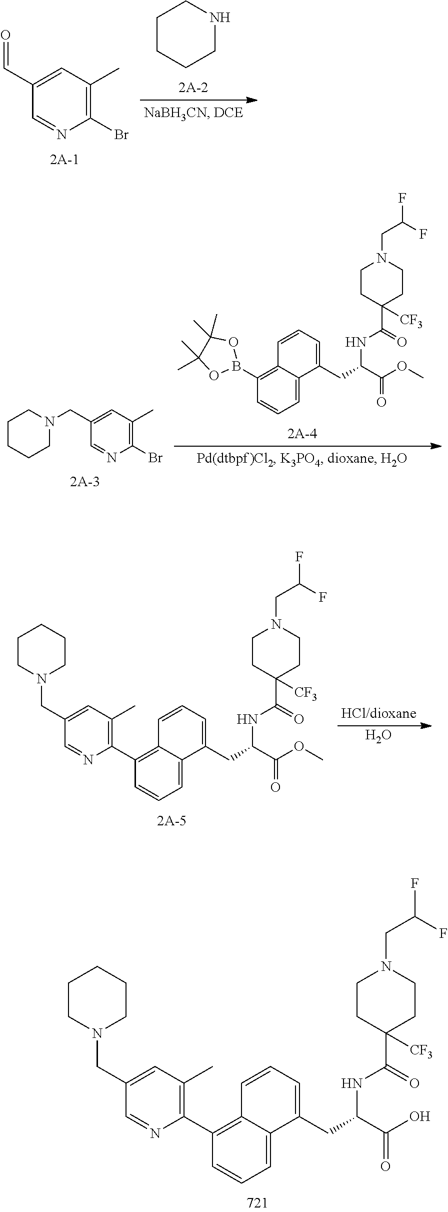

Step 1: To a solution of compound 2A-1 (100 mg, 499 μmol, 1.00 eq) was added compound 2A-2 (127 mg, 1.50 mmol, 148 μL, 3.00 eq) in Dichloromethane (2.00 mL). The mixture was stirred at 25° C. for 4 hrs, then NaBH₃CN (47.1 mg, 749 μmol, 1.50 eq) was added and the mixture was stirred at 25° C. for 1 hrs. The reaction mixture was diluted with H₂O 20.0 mL and extracted with ethyl acetate 15.0 mL (5.00 mL*3). The combined organic layers were concentrated under reduced pressure to give a residue. The residue was purified by Prep-TLC (SiO₂, Petroleum ether/Ethyl acetate=1/1; R_{f}=0.44). Compound 2A-3 (100 mg, 371 μmol, 74.3% yield) was obtained as a yellow oil. LC-MS: (M+H)⁺: 269.1.

Step 2: A mixture of compound 2A-3 (122 mg, 204 μmol, 1.10 eq), compound 2A-4 (50.0 mg, 185 μmol, 1.00 eq), Pd(dtbpf)Cl₂ (24.2 mg, 37.1 μmol, 0.200 eq), K₃PO₄ (78.8 mg, 371 μmol, 2.00 eq) in dioxane (2.00 mL) and H₂O (0.500 mL) was degassed and purged with N₂ for 3 times, and then the mixture was stirred at 25° C. for 4 hrs under N₂ atmosphere. The reaction mixture was diluted with H₂O 20 mL and extracted with ethyl acetate 15.0 mL (5.00 mL*3). The combined organic layers were concentrated under reduced pressure to give a residue. The residue was purified by Prep-TLC (SiO₂, Dichloromethane:Methanol=30:1; R_{f}=0.23). Compound 2A-5 (80.0 mg, 121 μmol, 65.1% yield) was obtained as a yellow oil. LC-MS: (M+H)⁺: 661.2.

Step 3: To a solution of compound 2A-5 (68.0 mg, 103 μmol, 1.00 eq) in MeOH (2.00 mL) and H₂O (0.500 mL) was added LiOH·H₂O (6.48 mg, 154 μmol, 1.50 eq). The mixture was stirred at 25° C. for 2 hrs. The reaction mixture was concentrated under reduced pressure to give a residue. The residue was purified by Prep-HPLC (column: Waters xbridge 150*25 mm 10 um; mobile phase: [water (NH₄HCO₃)-ACN]; gradient: 18%-48% B over 14 min). Compound 721 (52.49 mg, 78.4 μmol, 76.2% yield, 96.6% purity) was obtained as a white solid. LC-MS: (M+H)⁺: 647.4. ¹H NMR: (400 MHz, DMSO-d₆) δ 8.44-8.34 (m, 2H), 8.21 (d, J=9.2 Hz, 1H), 7.72-7.63 (m, 2H), 7.41 (d, J=7.2 Hz, 2H), 7.30 (t, J=8.0 Hz, 1H), 7.14 (d, J=8.4 Hz, 1H), 6.21-5.81 (m, 1H), 4.82-4.66 (m, 1H), 3.88-3.71 (m, 1H), 3.59-3.41 (m, 5H), 2.82-2.57 (m, 4H), 2.41 (br s, 2H), 2.27-2.14 (m, 2H), 2.04-1.72 (m, 5H), 1.65-1.49 (m, 6H), 1.47-1.35 (m, 2H).

### Synthesis of Compound 722

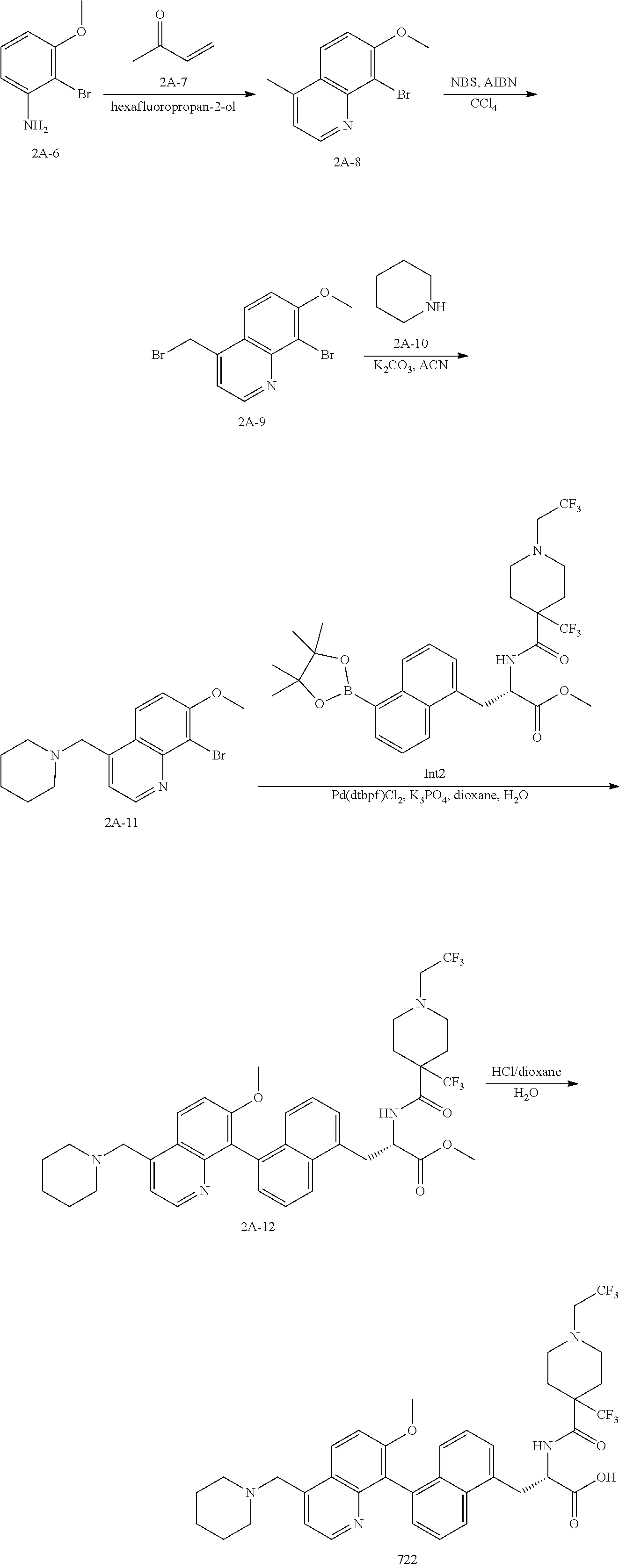

Step 1: To a solution of compound 2A-6 (15.0 g, 74.2 mmol, 1.00 eq) in HFIP (75.0 mL) was added compound 2A-7 (19.3 g, 276 mmol, 23.0 mL, 3.72 eq). The mixture was stirred at 60° C. for 6 hrs. The reaction mixture was filtered and concentrated under reduced pressure to give a residue. The residue was purified by column chromatography. Compound 2A-8 (5.00 g, crude) was obtained as a white solid. LC-MS: (M+H)⁺: 254.0. ¹H NMR: (400 MHz, CDCl₃) $8.81 (d, J=4.4 Hz, 1H), 7.92 (d, J=9.2 Hz, 1H), 7.28 (d, J=9.2 Hz, 1H), 7.18 (d, J=4.4 Hz, 1H), 4.01 (s, 3H), 2.03 (s, 3H).

Step 2: To a solution of compound 2A-8 (300 mg, 1.19 mmol, 1.00 eq) in CHCl₃ (5.00 mL) was added NBS (211 mg, 1.19 mmol, 1.00 eq) and AIBN (195 mg, 1.19 mmol, 1.00 eq). The mixture was stirred at 60° C. for 3 hrs. The reaction mixture was diluted with H₂O 10.0 mL and extracted with ethyl acetate 30.0 mL (10.0 mL*3). The combined organic layers concentrated under reduced pressure to give a residue. The residue was purified by Prep-TLC (SiO₂, Petroleum ether:Ethyl acetate=5:2, R_{f}=0.38). Compound 2A-9 (120 mg, 362 μmol, 30.4% yield) was obtained as a yellow oil. LC-MS: (M+H)⁺: 331.8. ¹H NMR: (400 MHz, DMSO-d₆) δ 8.92 (d, J=4.4 Hz, 1H), 8.30 (d, J=9.2 Hz, 1H), 7.72 (d, J=9.2 Hz, 1H), 7.32 (d, J=4.4 Hz, 1H), 5.20 (s, 2H), 4.05 (s, 3H).

Step 3: A mixture of compound 2A-9 (61.7 mg, 725 μmol, 71.6 μL, 2.00 eq), compound 2A-10 (120 mg, 362 μmol, 1.00 eq), K₂CO₃ (100 mg, 725 μmol, 2.00 eq), in ACN (1.00 mL) was degassed and purged with N₂ for 3 times, and then the mixture was stirred at 25° C. for 1 hr under N₂ atmosphere. The reaction mixture was diluted with H₂O 10.0 mL and extracted with ethyl acetate 30.0 mL (10.0 mL*3). The combined organic layers was concentrated under reduced pressure to give a residue. The residue was purified by Prep-TLC (SiO₂, Petroleum ether:Ethyl acetate=3:2, R_{f}-0.50). Compound 2A-11 (160 mg, 343 μmol, 94.7% yield, 72.0% purity) was obtained as a yellow oil. LC-MS: (M+H)⁺: 335.0.

Step 4: A mixture of compound 2A-11 (317 mg, 509 μmol, 1.50 eq), compound Int2 (158 mg, 339 μmol, 1.00 eq), Pd(dtbpf)Cl₂ (22.1 mg, 33.9 μmol, 0.100 eq), K₃PO₄ (144 mg, 678 μmol, 2.00 eq) in dioxane (4.00 mL) and H₂O (1.00 mL) was degassed and purged with N₂ for 3 times, and then the mixture was stirred at 40° C. for 4 hrs under N₂ atmosphere. The reaction mixture was diluted with H₂O 10.0 mL and extracted with ethyl acetate 30.0 mL (10.0 mL*3). The combined organic layers were concentrated under reduced pressure to give a residue. The residue was purified by Prep-TLC (SiO₂, Ethyl acetate:Methanol=10:1, R_{f}=0.47). Compound 2A-12 (200 mg, 176 μmol, 51.9% yield, 65.7% purity) was obtained as a yellow oil. LC-MS: (M+H)⁺: 745.5.

Step 5: To a solution of compound 2A-12 (200 mg, 176 μmol, 1.00 eq) in H₂O (1.00 mL) was added HCl/dioxane (4.00 M, 1.33 mL, 30.0 eq). The mixture was stirred at 60° C. for 2 hrs. The reaction mixture was concentrated under reduced pressure to give a residue. The residue was purified by Prep-HPLC (column: Welch Xtimate C18 150*25 mm*5 um; mobile phase: [water (FA)-ACN]; gradient: 15%-45% B over 10 min). Compound 722 (82.83 mg, 112 μmol, 63.9% yield, 99.5% purity) was obtained as a white solid. LC-MS: (M+H)⁺: 731.2. ¹H NMR: (400 MHz, DMSO-d₆). δ 13.07-12.77 (m, 1H), 8.49-8.42 (m, 3H), 8.14 (d, J=10.0 Hz, 1H), 7.73-7.61 (m, 2H), 7.34-7.25 (m, 3H), 7.13-7.10 (m, 1H), 7.00 (t, J=9.2 Hz, 1H), 4.82-4.70 (m, 1H), 3.97-3.85 (m, 3H), 3.77-3.71 (m, 3H), 3.48-3.31 (m, 1H), 2.98-2.81 (m, 2H), 2.75-2.59 (m, 3H), 2.50-2.46 (m, 3H), 2.33-2.25 (m, 2H), 2.12 (t, J=12.4 Hz, 1H), 2.00-1.92 (m, 1H), 1.64-1.52 (m, 6H), 1.44-1.41 (m, 2H).

The compounds set forth in Table 20, were prepared using the synthetic procedures of Example 40, or analogous procedures as provided herein.

TABLE 20
Structure and Spectroscopic data for Compounds 723 to 739 and 741 to 777

| No. | Structure |
|---|---|
| 723 | 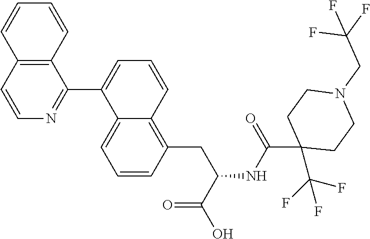 [SMILES: O=C(O)[C@H](Cc1cccc2c(-c3nccc4ccccc34)cccc12)NC(=O)C1(C(F)(F)F)CCN(CC(F)(F)F)CC1] |
| | m/z = 602.1 |
| 724 | 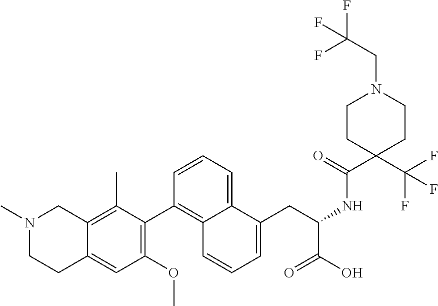 [SMILES: COc1cc2c(c(C)c1-c1cccc3c(C[C@H](NC(=O)C4(C(F)(F)F)CCN(CC(F)(F)F)CC4)C(=O)O)cccc13)CN(C)CC2] |
| | m/z = 666 |
| 725 | 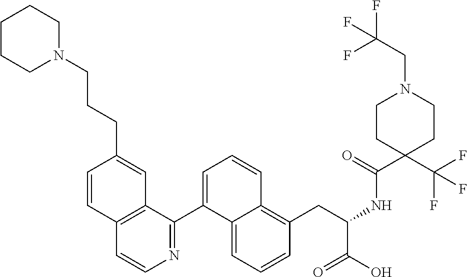 [SMILES: O=C(O)[C@H](Cc1cccc2c(-c3nccc4ccc(CCCN5CCCCC5)cc34)cccc12)NC(=O)C1(C(F)(F)F)CCN(CC(F)(F)F)CC1] |
| | m/z = 729.5 |
| 726 | 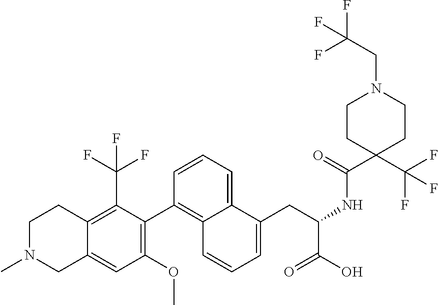 [SMILES: COc1cc2c(c(C(F)(F)F)c1-c1cccc3c(C[C@H](NC(=O)C4(C(F)(F)F)CCN(CC(F)(F)F)CC4)C(=O)O)cccc13)CCN(C)C2] |
| | m/z = 720.2 |
| 727 | 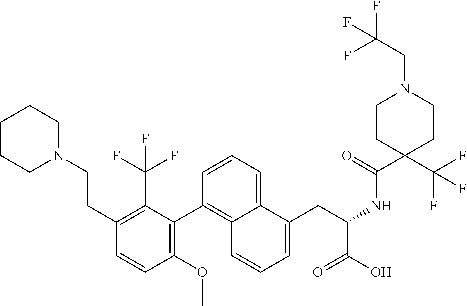 [SMILES: COc1ccc(CCN2CCCCC2)c(C(F)(F)F)c1-c1cccc2c(C[C@H](NC(=O)C3(C(F)(F)F)CCN(CC(F)(F)F)CC3)C(=O)O)cccc12] |
| | m/z = 762.2 |
| 728 | 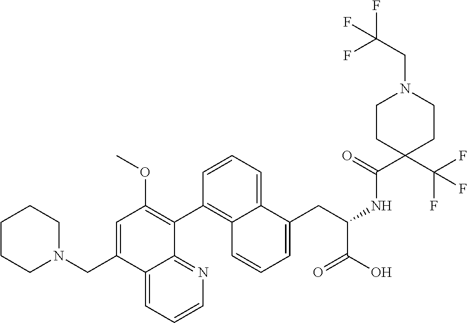 [SMILES: COc1cc(CN2CCCCC2)c2cccnc2c1-c1cccc2c(C[C@H](NC(=O)C3(C(F)(F)F)CCN(CC(F)(F)F)CC3)C(=O)O)cccc12] |
| | m/z = 731.5 |
| 729 | 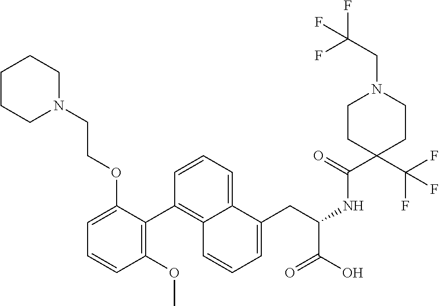 [SMILES: COc1cccc(OCCN2CCCCC2)c1-c1cccc2c(C[C@H](NC(=O)C3(C(F)(F)F)CCN(CC(F)(F)F)CC3)C(=O)O)cccc12] |
| | m/z = 710.3 |
| 730 | 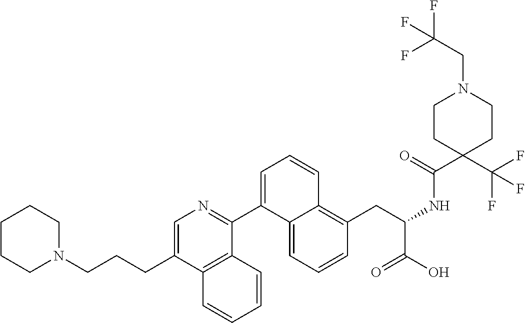 [SMILES: O=C(O)[C@H](Cc1cccc2c(-c3ncc(CCCN4CCCCC4)c4ccccc34)cccc12)NC(=O)C1(C(F)(F)F)CCN(CC(F)(F)F)CC1] |
| | m/z = 729.4 |
| 731 | 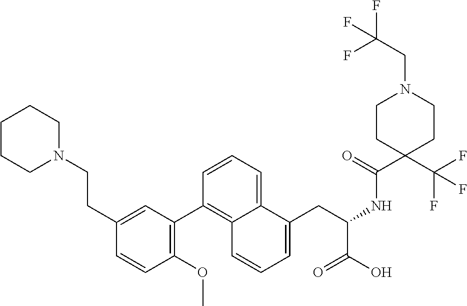 [SMILES: COc1ccc(CCN2CCCCC2)cc1-c1cccc2c(C[C@H](NC(=O)C3(C(F)(F)F)CCN(CC(F)(F)F)CC3)C(=O)O)cccc12] |
| | m/z = 694.4 |
| 732 | 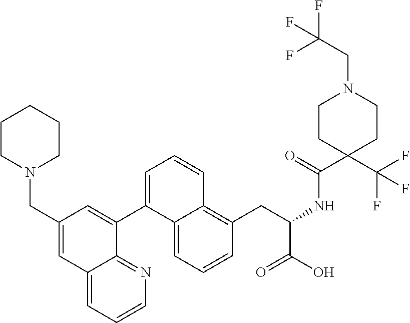 [SMILES: O=C(O)[C@H](Cc1cccc2c(-c3cc(CN4CCCCC4)cc4cccnc34)cccc12)NC(=O)C1(C(F)(F)F)CCN(CC(F)(F)F)CC1] |
| | m/z = 701.2 |
| 733 | 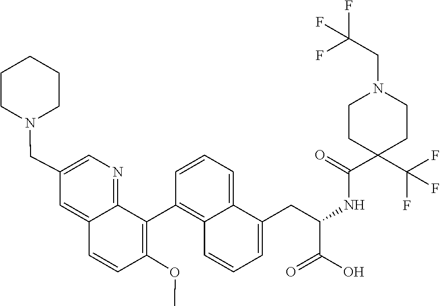 [SMILES: COc1ccc2cc(CN3CCCCC3)cnc2c1-c1cccc2c(C[C@H](NC(=O)C3(C(F)(F)F)CCN(CC(F)(F)F)CC3)C(=O)O)cccc12] |
| | m/z = 731.1 |
| 734 | 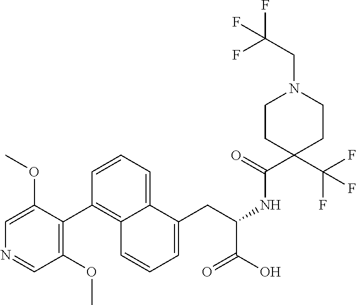 [SMILES: COc1cncc(OC)c1-c1cccc2c(C[C@H](NC(=O)C3(C(F)(F)F)CCN(CC(F)(F)F)CC3)C(=O)O)cccc12] |
| | m/z = 614.1 |
| 735 | 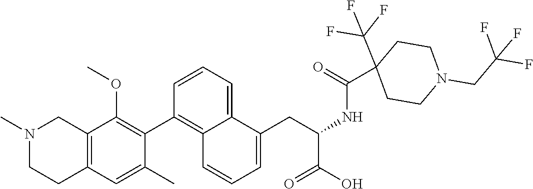 [SMILES: COc1c2c(cc(C)c1-c1cccc3c(C[C@H](NC(=O)C4(C(F)(F)F)CCN(CC(F)(F)F)CC4)C(=O)O)cccc13)CCN(C)C2] |
| | m/z = 666.4 |
| 736 | 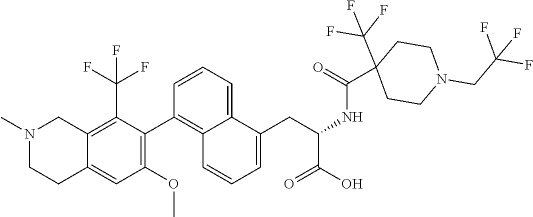 [SMILES: COc1cc2c(c(C(F)(F)F)c1-c1cccc3c(C[C@H](NC(=O)C4(C(F)(F)F)CCN(CC(F)(F)F)CC4)C(=O)O)cccc13)CN(C)CC2] |
| | m/z = 720.2 |
| 737 | 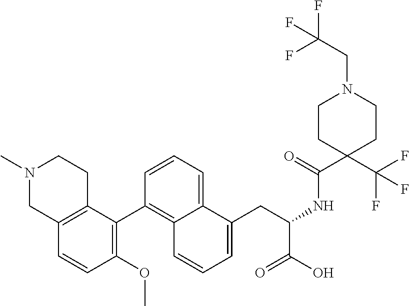 [SMILES: COc1ccc2c(c1-c1cccc3c(C[C@H](NC(=O)C4(C(F)(F)F)CCN(CC(F)(F)F)CC4)C(=O)O)cccc13)CCN(C)C2] |
| | m/z = 652.2 |
| 738 | 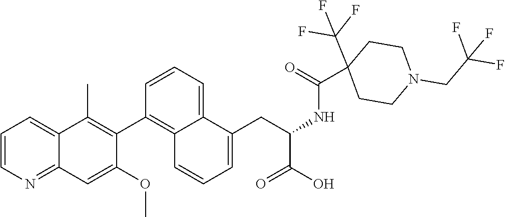 [SMILES: COc1cc2ncccc2c(C)c1-c1cccc2c(C[C@H](NC(=O)C3(C(F)(F)F)CCN(CC(F)(F)F)CC3)C(=O)O)cccc12] |
| | m/z = 648.3 |
| 739 | 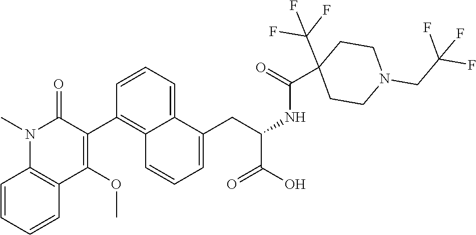 [SMILES: COc1c(-c2cccc3c(C[C@H](NC(=O)C4(C(F)(F)F)CCN(CC(F)(F)F)CC4)C(=O)O)cccc23)c(=O)n(C)c2ccccc12] |
| | m/z = 664.2 |
| 741 | 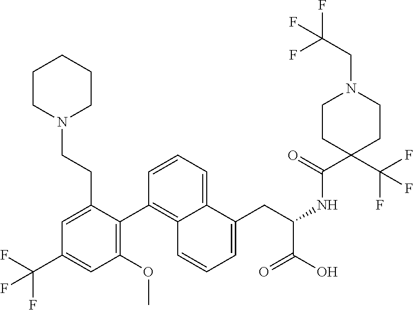 [SMILES: COc1cc(C(F)(F)F)cc(CCN2CCCCC2)c1-c1cccc2c(C[C@H](NC(=O)C3(C(F)(F)F)CCN(CC(F)(F)F)CC3)C(=O)O)cccc12] |
| | m/z = 762.4 |
| 742 | 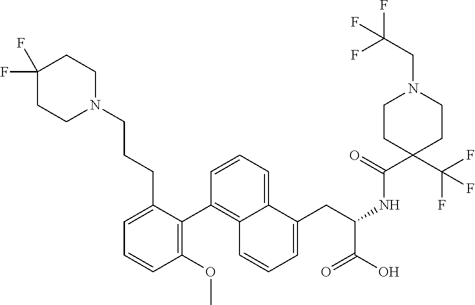 [SMILES: COc1cccc(CCCN2CCC(F)(F)CC2)c1-c1cccc2c(C[C@H](NC(=O)C3(C(F)(F)F)CCN(CC(F)(F)F)CC3)C(=O)O)cccc12] |
| | m/z = 744.2 |
| 743 | 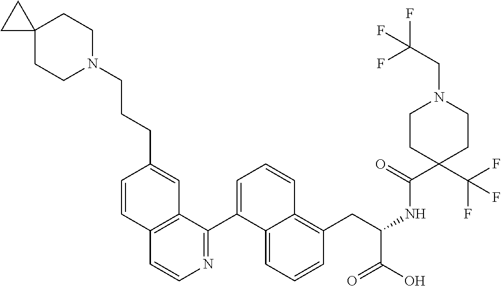 [SMILES: O=C(O)[C@H](Cc1cccc2c(-c3nccc4ccc(CCCN5CCC6(CC5)CC6)cc34)cccc12)NC(=O)C1(C(F)(F)F)CCN(CC(F)(F)F)CC1] |
| | m/z = 755.3 |
| 744 | 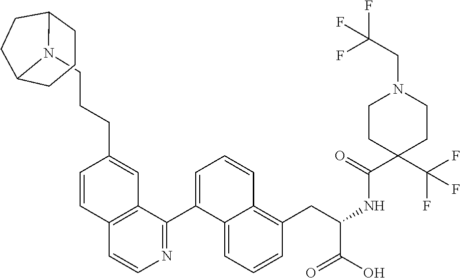 [SMILES: O=C(O)[C@H](Cc1cccc2c(-c3nccc4ccc(CCCN5C6CCCC5CC6)cc34)cccc12)NC(=O)C1(C(F)(F)F)CCN(CC(F)(F)F)CC1] |
| | m/z = 755.3 |
| 745 | 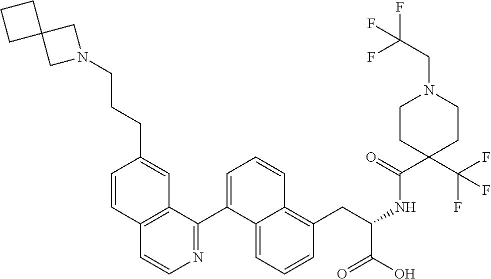 [SMILES: O=C(O)[C@H](Cc1cccc2c(-c3nccc4ccc(CCCN5CC6(CCC6)C5)cc34)cccc12)NC(=O)C1(C(F)(F)F)CCN(CC(F)(F)F)CC1] |
| | m/z = 741.4 |
| 746 | 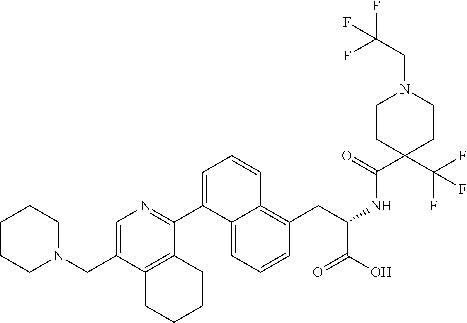 [SMILES: O=C(O)[C@H](Cc1cccc2c(-c3ncc(CN4CCCCC4)c4c3CCCC4)cccc12)NC(=O)C1(C(F)(F)F)CCN(CC(F)(F)F)CC1] |
| | m/z = 705.4 |
| 747 | 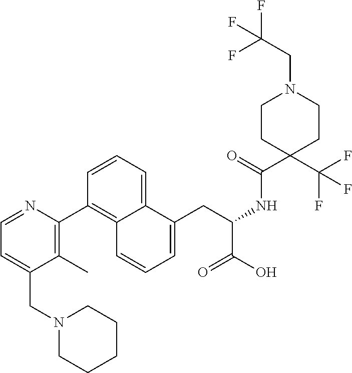 [SMILES: Cc1c(CN2CCCCC2)ccnc1-c1cccc2c(C[C@H](NC(=O)C3(C(F)(F)F)CCN(CC(F)(F)F)CC3)C(=O)O)cccc12] |
| | m/z = 665.3 |
| 748 | 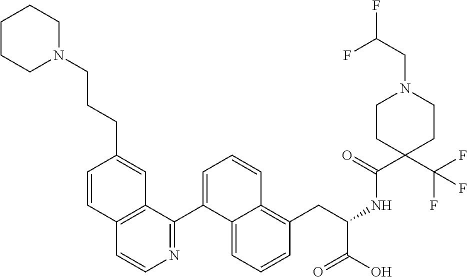 [SMILES: O=C(O)[C@H](Cc1cccc2c(-c3nccc4ccc(CCCN5CCCCC5)cc34)cccc12)NC(=O)C1(C(F)(F)F)CCN(CC(F)F)CC1] |
| | m/z = 711.2 |
| 749 | 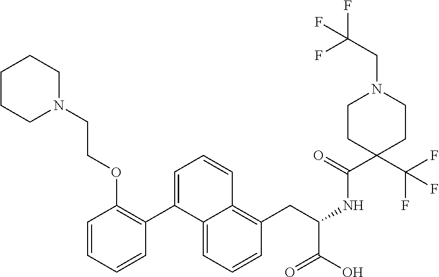 [SMILES: O=C(O)[C@H](Cc1cccc2c(-c3ccccc3OCCN3CCCCC3)cccc12)NC(=O)C1(C(F)(F)F)CCN(CC(F)(F)F)CC1] |
| | m/z = 680.3 |
| 750 | 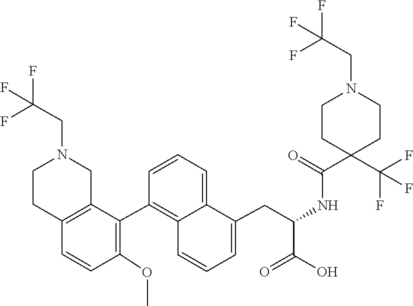 [SMILES: COc1ccc2c(c1-c1cccc3c(C[C@H](NC(=O)C4(C(F)(F)F)CCN(CC(F)(F)F)CC4)C(=O)O)cccc13)CN(CC(F)(F)F)CC2] |
| | m/z = 720.3 |
| 751 | 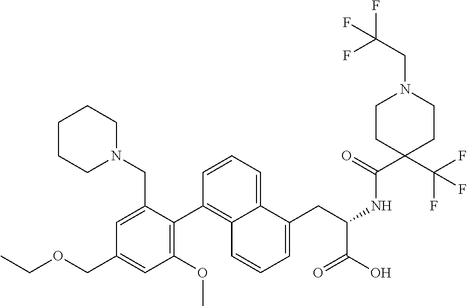 [SMILES: CCOCc1cc(CN2CCCCC2)c(-c2cccc3c(C[C@H](NC(=O)C4(C(F)(F)F)CCN(CC(F)(F)F)CC4)C(=O)O)cccc23)c(OC)c1] |
| | m/z = 738.5 |
| 752 | 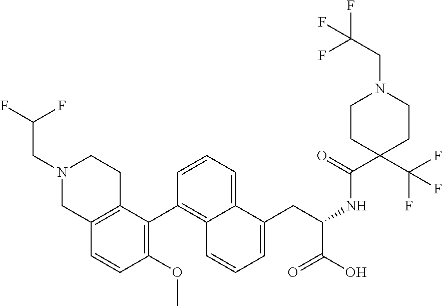 [SMILES: COc1ccc2c(c1-c1cccc3c(C[C@H](NC(=O)C4(C(F)(F)F)CCN(CC(F)(F)F)CC4)C(=O)O)cccc13)CCN(CC(F)F)C2] |
| | m/z = 702.2 |
| 753 | 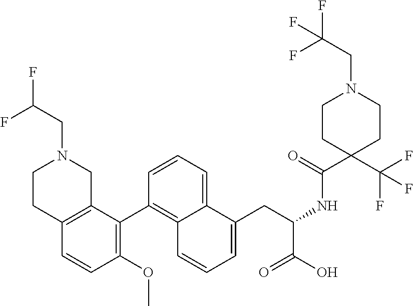 [SMILES: COc1ccc2c(c1-c1cccc3c(C[C@H](NC(=O)C4(C(F)(F)F)CCN(CC(F)(F)F)CC4)C(=O)O)cccc13)CN(CC(F)F)CC2] |
| | m/z = 702.4 |
| 754 | 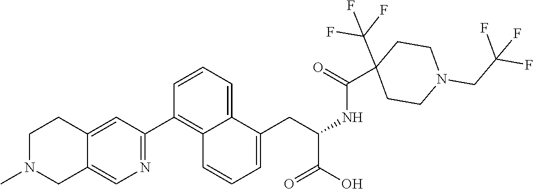 [SMILES: CN1CCc2cc(-c3cccc4c(C[C@H](NC(=O)C5(C(F)(F)F)CCN(CC(F)(F)F)CC5)C(=O)O)cccc34)ncc2C1] |
| | m/z = 623.1 |
| 755 | 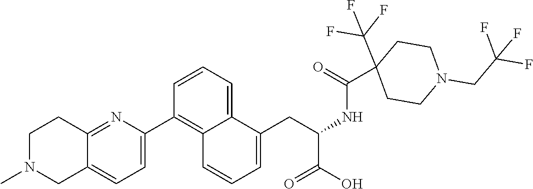 [SMILES: CN1CCc2nc(-c3cccc4c(C[C@H](NC(=O)C5(C(F)(F)F)CCN(CC(F)(F)F)CC5)C(=O)O)cccc34)ccc2C1] |
| | m/z = 623.2 |
| 756 | 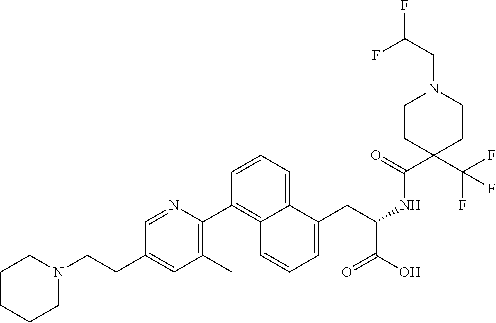 [SMILES: Cc1cc(CCN2CCCCC2)cnc1-c1cccc2c(C[C@H](NC(=O)C3(C(F)(F)F)CCN(CC(F)F)CC3)C(=O)O)cccc12] |
| | m/z = 661.3 |
| 757 | 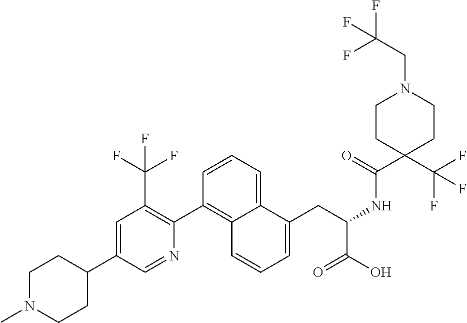 [SMILES: CN1CCC(c2cnc(-c3cccc4c(C[C@H](NC(=O)C5(C(F)(F)F)CCN(CC(F)(F)F)CC5)C(=O)O)cccc34)c(C(F)(F)F)c2)CC1] |
| | m/z = 719.2 |
| 758* | 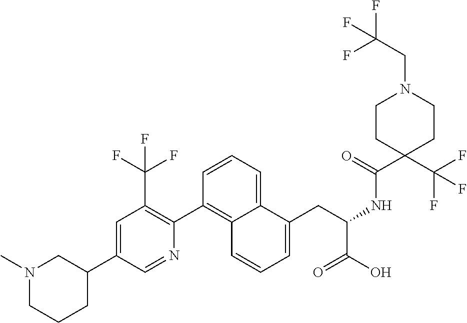 [SMILES: CN1CCCC(c2cnc(-c3cccc4c(C[C@H](NC(=O)C5(C(F)(F)F)CCN(CC(F)(F)F)CC5)C(=O)O)cccc34)c(C(F)(F)F)c2)C1] |
| | m/z = 719.4 |
| 759 | 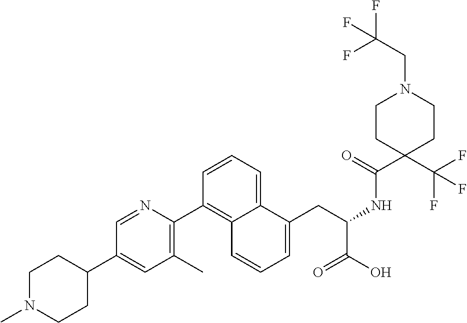 [SMILES: Cc1cc(C2CCN(C)CC2)cnc1-c1cccc2c(C[C@H](NC(=O)C3(C(F)(F)F)CCN(CC(F)(F)F)CC3)C(=O)O)cccc12] |
| | m/z = 665.2 |
| 760* | 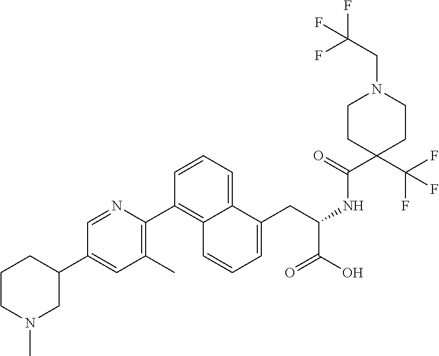 [SMILES: Cc1cc(C2CCCN(C)C2)cnc1-c1cccc2c(C[C@H](NC(=O)C3(C(F)(F)F)CCN(CC(F)(F)F)CC3)C(=O)O)cccc12] |
| | m/z = 665.2 |
| 761 | 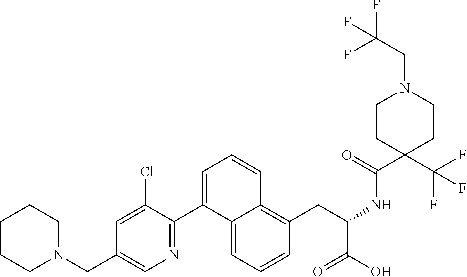 [SMILES: O=C(O)[C@H](Cc1cccc2c(-c3ncc(CN4CCCCC4)cc3Cl)cccc12)NC(=O)C1(C(F)(F)F)CCN(CC(F)(F)F)CC1] |
| | m/z = 685.1 |
| 762 | 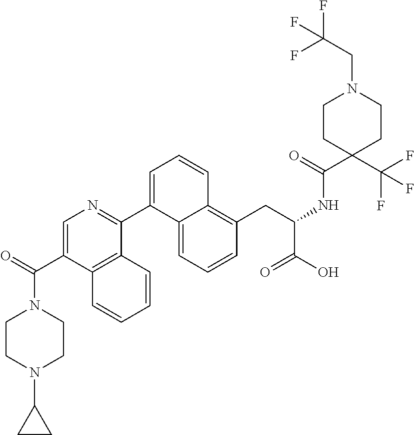 [SMILES: O=C(O)[C@H](Cc1cccc2c(-c3ncc(C(=O)N4CCN(C5CC5)CC4)c4ccccc34)cccc12)NC(=O)C1(C(F)(F)F)CCN(CC(F)(F)F)CC1] |
| | m/z = 756.4 |
| 763 | 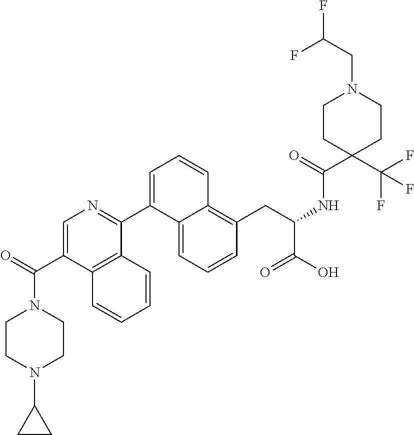 [SMILES: O=C(O)[C@H](Cc1cccc2c(-c3ncc(C(=O)N4CCN(C5CC5)CC4)c4ccccc34)cccc12)NC(=O)C1(C(F)(F)F)CCN(CC(F)F)CC1] |
| | m/z = 738.3 |
| 764 | 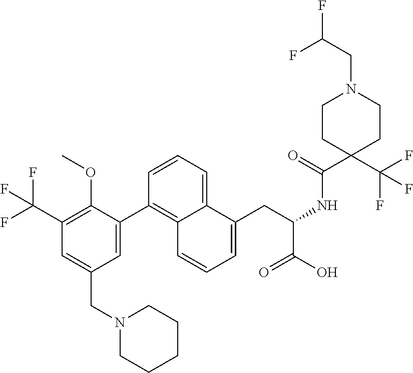 [SMILES: COc1c(-c2cccc3c(C[C@H](NC(=O)C4(C(F)(F)F)CCN(CC(F)F)CC4)C(=O)O)cccc23)cc(CN2CCCCC2)cc1C(F)(F)F] |
| | m/z = 730.3 |
| 765 | 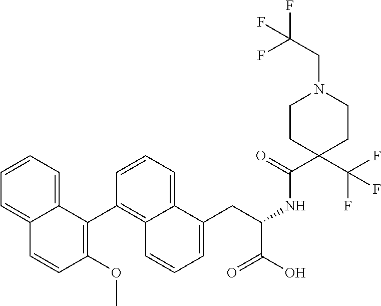 [SMILES: COc1ccc2ccccc2c1-c1cccc2c(C[C@H](NC(=O)C3(C(F)(F)F)CCN(CC(F)(F)F)CC3)C(=O)O)cccc12] |
| | m/z = 633.2 |
| 766 | 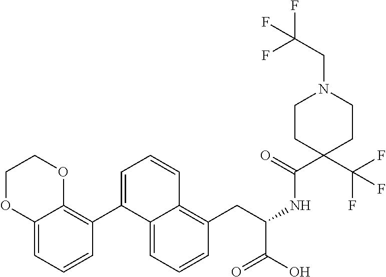 [SMILES: O=C(O)[C@H](Cc1cccc2c(-c3cccc4c3OCCO4)cccc12)NC(=O)C1(C(F)(F)F)CCN(CC(F)(F)F)CC1] |
| | m/z = 611.2 |
| 767 | 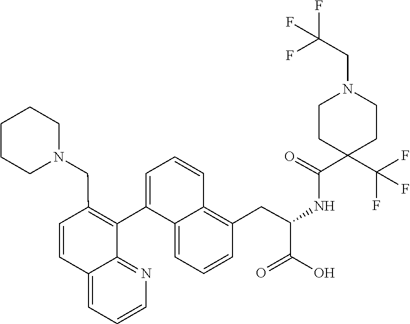 [SMILES: O=C(O)[C@H](Cc1cccc2c(-c3c(CN4CCCCC4)ccc4cccnc34)cccc12)NC(=O)C1(C(F)(F)F)CCN(CC(F)(F)F)CC1] |
| | m/z = 701.2 |
| 768 | 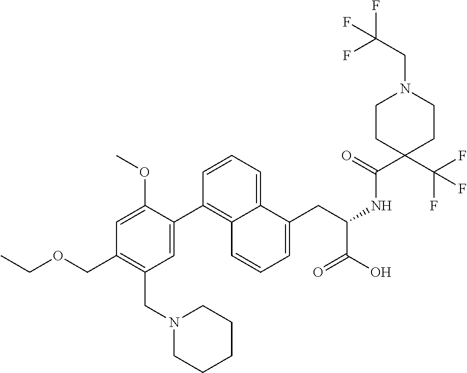 [SMILES: CCOCc1cc(OC)c(-c2cccc3c(C[C@H](NC(=O)C4(C(F)(F)F)CCN(CC(F)(F)F)CC4)C(=O)O)cccc23)cc1CN1CCCCC1] |
| | m/z = 738.4 |
| 769 | 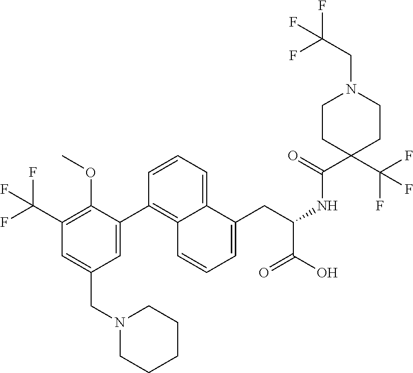 [SMILES: COc1c(-c2cccc3c(C[C@H](NC(=O)C4(C(F)(F)F)CCN(CC(F)(F)F)CC4)C(=O)O)cccc23)cc(CN2CCCCC2)cc1C(F)(F)F] |
| | m/z = 748.2 |
| 770 | 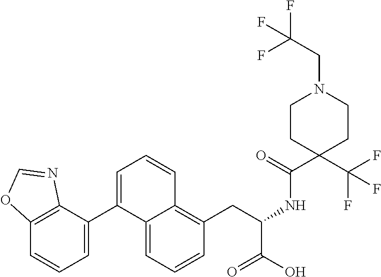 [SMILES: O=C(O)[C@H](Cc1cccc2c(-c3cccc4ocnc34)cccc12)NC(=O)C1(C(F)(F)F)CCN(CC(F)(F)F)CC1] |
| | m/z = 594.2 |
| 771 | 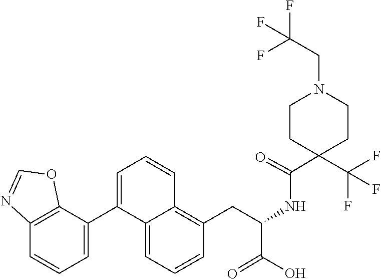 [SMILES: O=C(O)[C@H](Cc1cccc2c(-c3cccc4ncoc34)cccc12)NC(=O)C1(C(F)(F)F)CCN(CC(F)(F)F)CC1] |
| | m/z = 594 |
| 772 | 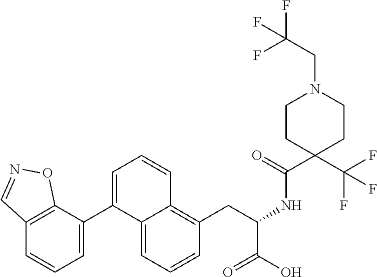 [SMILES: O=C(O)[C@H](Cc1cccc2c(-c3cccc4cnoc34)cccc12)NC(=O)C1(C(F)(F)F)CCN(CC(F)(F)F)CC1] |
| | m/z = 594.2 |
| 773 | 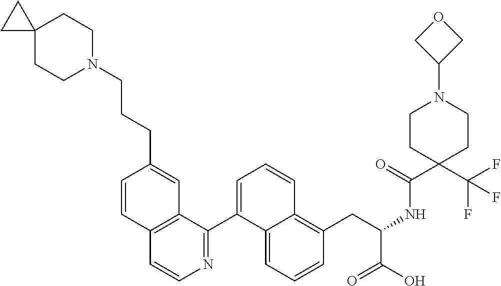 [SMILES: O=C(O)[C@H](Cc1cccc2c(-c3nccc4ccc(CCCN5CCC6(CC5)CC6)cc34)cccc12)NC(=O)C1(C(F)(F)F)CCN(C2COC2)CC1] |
| | m/z = 729.1 |
| 774 | 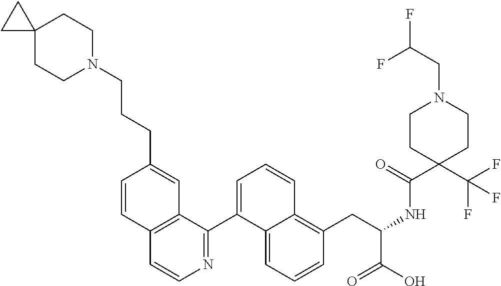 [SMILES: O=C(O)[C@H](Cc1cccc2c(-c3nccc4ccc(CCCN5CCC6(CC5)CC6)cc34)cccc12)NC(=O)C1(C(F)(F)F)CCN(CC(F)F)CC1] |
| | m/z = 737 |
| 775 | 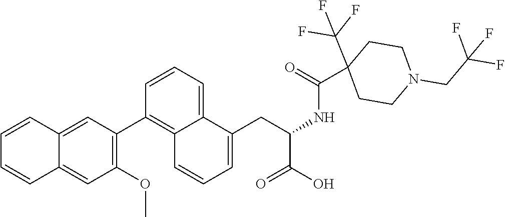 [SMILES: COc1cc2ccccc2cc1-c1cccc2c(C[C@H](NC(=O)C3(C(F)(F)F)CCN(CC(F)(F)F)CC3)C(=O)O)cccc12] |
| | m/z = 633.1 |
| 776 | 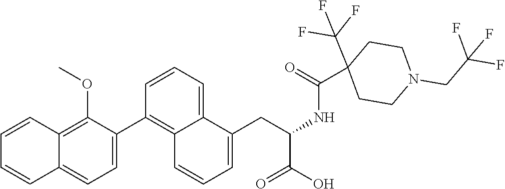 [SMILES: COc1c(-c2cccc3c(C[C@H](NC(=O)C4(C(F)(F)F)CCN(CC(F)(F)F)CC4)C(=O)O)cccc23)ccc2ccccc12] |
| | m/z = 633.1 |
| 777 | 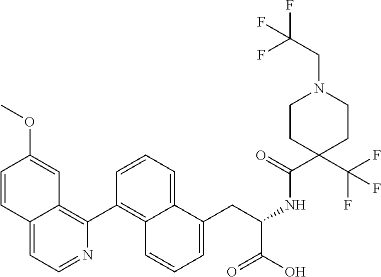 [SMILES: COc1ccc2ccnc(-c3cccc4c(C[C@H](NC(=O)C5(C(F)(F)F)CCN(CC(F)(F)F)CC5)C(=O)O)cccc34)c2c1] |
| | m/z = 634.1 |

*Denotes a racemic compound;
**Denotes a single isomer with unknown absolute stereochemistry

### Example 41: General Scheme of 2B

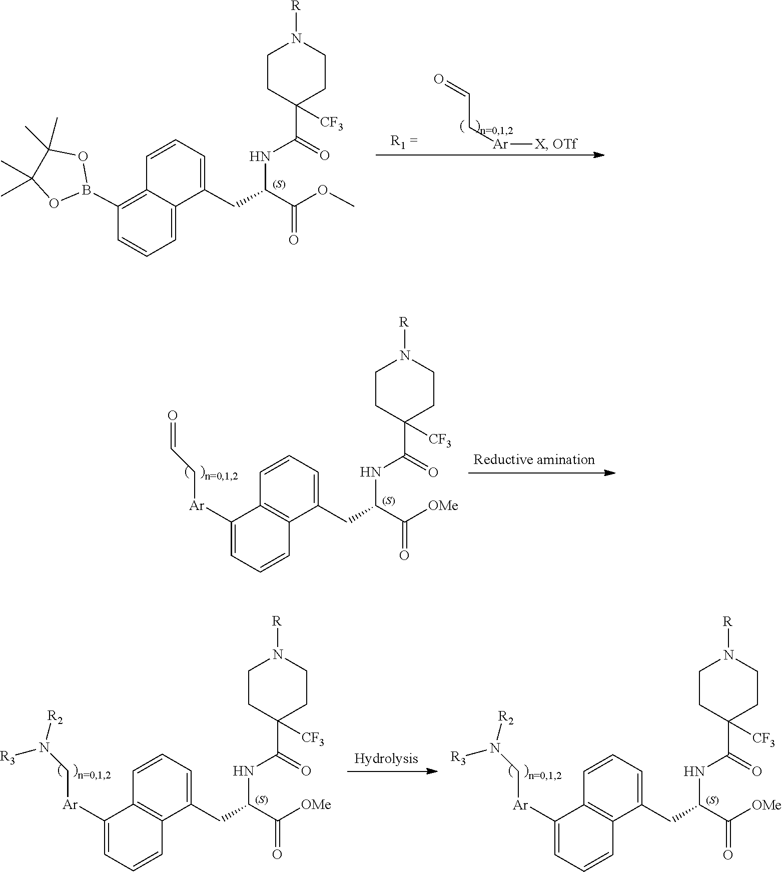

The proceeding compounds were synthesized according to the procedures described in General Scheme 2B using Common Intermediate 2 or Int2 as an intermediate.

### Synthesis of Compound 778

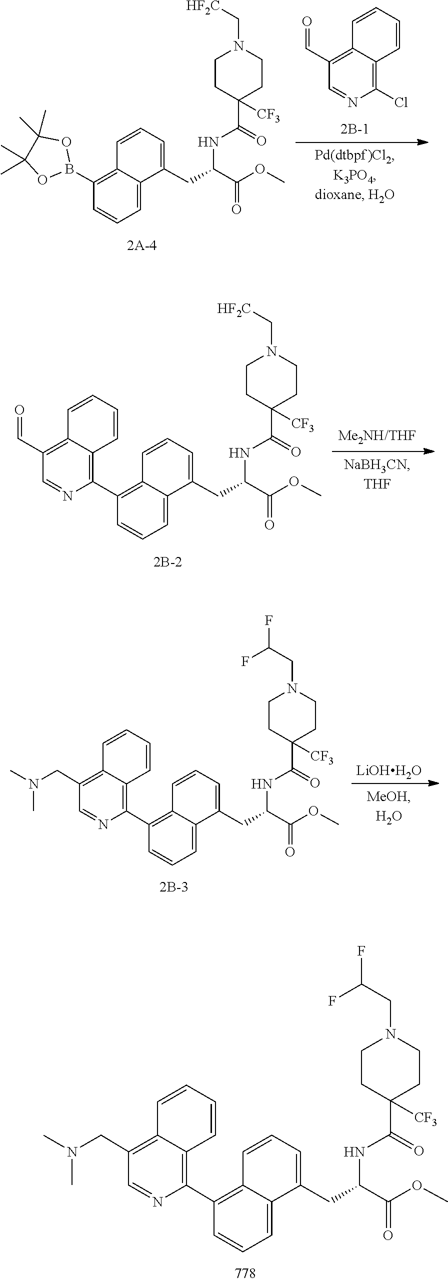

Step 1: A mixture of compound 2A-4 (1.50 g, 2.51 mmol, 1.00 eq), compound 2B-1 (480 mg, 2.51 mmol, 1.00 eq), K₃PO₄ (1.33 g, 6.27 mmol, 2.50 eq), Pd(dtbpf)Cl₂ (163 mg, 250 μmol, 0.100 eq) in dioxane (20.0 mL), H₂O (4.00 mL) was degassed and purged with N₂ for 3 times, and then the mixture was stirred at 25° C. for 1 hr under N₂ atmosphere. The reaction mixture was partitioned between H₂O 30.0 mL and ethyl acetate 30.0*3 mL. The organic phase was separated, washed with brine (100 mL*2), concentrated under reduced pressure to give a residue. The residue was purified by column chromatography (SiO₂, Petroleum ether/Ethyl acetate=10/1 to 3/1). Compound 2B-2 (1.50 g, 2.39 mmol, 95.3% yield) was obtained as yellow solid. LC-MS: (M+H)⁺: 628.2.

Step 2: To a solution of compound 2B-2 (80.0 mg, 127 μmol, 1.00 eq), Me₂NH/THF (2.00 M, 191 μL, 3.00 eq) in THF (0.500 mL) was stirred at 25° C. for 2 hrs, then NaBH₃CN (14.4 mg, 229 μmol, 1.80 eq) was added and keep stirred for 0.5 hr. The reaction mixture was diluted with H₂O 5.00 mL, and extracted with DCM (5.00 mL*4). The combined organic layers were concentrated under reduced pressure to give a residue. The residue was purified by Prep-TLC (SiO₂, Dichloromethane/Methanol=10/1). Compound 2B-3 (60.0 mg, 91.3 μmol, 71.6% yield) was obtained as a white solid. LC-MS: (M+H)⁺: 657.3.

Step 3: To a solution of compound 2B-3 (50.0 mg, 76.1 μmol, 1.00 eq) in MeOH (0.500 mL), H₂O (0.500 mL) was added LiOH·H₂O (5.75 mg, 137 μmol, 1.80 eq) and stirred at 25° C. for 0.5 hr. The reaction mixture was filtered to give a residue. The residue was purified by Prep-HPLC (column: Waters xbridge 150*25 mm 10 um; mobile phase: [water (NH₄HCO₃)-ACN]; gradient: 25%-55% B over 10 min). Compound 778 (38.71 mg, 58.8 μmol, 77.2% yield, 97.7% purity) was obtained as a white solid. LC-MS: (M+H)⁺: 643.0. ¹H NMR: (400 MHz, DMSO-d₆) δ 8.52 (d, J=1.2 Hz, 1H), 8.47-8.36 (m, 2H), 8.33 (t, J=8.8 Hz, 1H), 7.84-7.71 (m, 2H), 7.56 (d, J=6.8 Hz, 1H), 7.51-7.34 (m, 3H), 7.25-7.20 (m, 1H), 7.06-7.03 (m, 1H), 6.15-5.82 (m, 1H), 4.89-4.72 (m, 1H), 3.87 (s, 2H), 3.85 (d, J=3.2 Hz, 1H), 3.54-3.48 (m, 1H), 2.75-2.56 (m, 2H), 2.49-2.31 (m, 2H), 2.28 (s, 6H), 2.25-2.22 (m, 2H), 1.97-1.80 (m, 1H), 1.78-1.60 (m, 1H), 1.60-1.48 (m, 2H).

### Synthesis of Compound 779

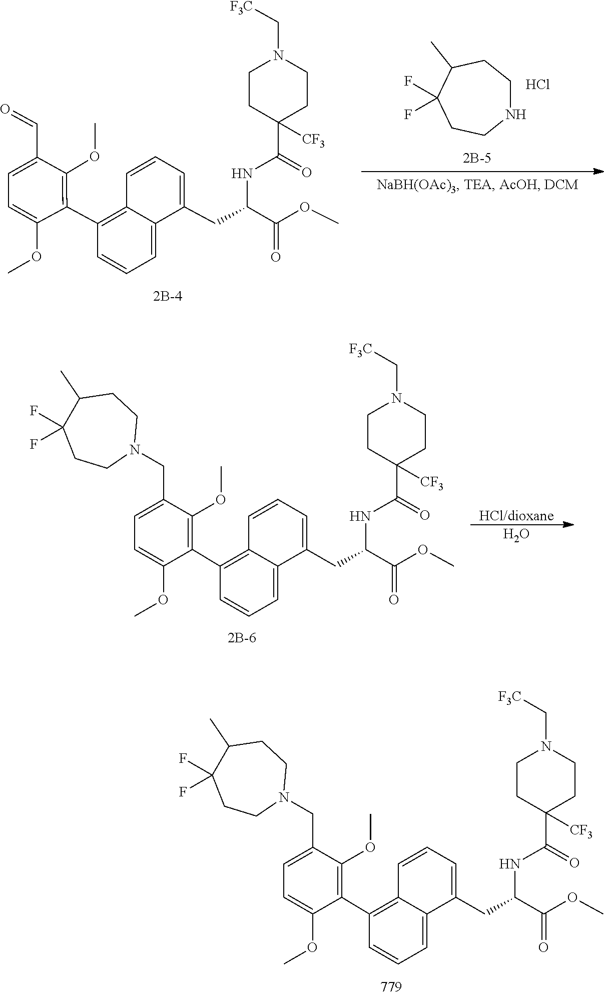

Step 1: To a solution of compound 2B-4 (90.0 mg, 128 μmol, 1.00 eq) and compound 2B-5 (47.6 mg, 256 μmol, 2.00 eq, HCl) in DCM (2.00 mL) was added NaBH(OAc)₃ (81.5 mg, 384 μmol, 3.00 eq), TEA (25.9 mg, 256 μmol, 35.7 μL, 2.00 eq) and AcOH (7.70 mg, 128 μmol, 7.34 μL, 1.00 eq). The mixture was stirred at 25° C. for 6 hrs. The residue was diluted with H₂O 10.0 mL and extracted with DCM 60.0 mL (20.0 mL*3). The combined organic layers were washed with brine 10.0 mL (10.0 mL*1), dried over Na₂SO₄, filtered, and concentrated under reduced pressure to give a residue. The residue was purified by Prep-TLC (SiO₂, DCM:MeOH=10:1, R_{f}=0.67). Compound 2B-6 (91.0 mg, crude) was obtained as yellow oil. LC-MS: (M+23)⁺: 810.4.

Step 2: To a solution of compound 2B-6 (81.0 mg, 103 μmol, 1.00 eq) in H₂O (0.500 mL) was added HCl/dioxane (4.00 M, 1.46 mL, 56.9 eq). The mixture was stirred at 60° C. for 2 hrs. The reaction mixture was concentrated under reduced pressure to give a residue. The residue was purified by Prep-HPLC (column: Phenomenex C18 150*25 mm*10 um; mobile phase: [water (NH₄HCO₃)-ACN]; gradient: 37%-67% B over 14 min). Compound 779 (39.99 mg, 50.6 μmol, 49.2% yield, 97.9% purity) was obtained as a white solid. LC-MS: (M+H)⁺: 774.4. ¹H NMR: (400 MHz, DMSO-d₆) δ 8.48-8.35 (m, 1H), 8.14 (d, J=8.4 Hz, 1H), 7.67-7.59 (m, 1H), 7.44 (d, J=8.4 Hz, 1H), 7.40-7.33 (m, 2H), 7.30-7.18 (m, 2H), 6.94 (dd, J₁=8.4 Hz, J₂=2.8 Hz, 1H), 4.86-4.67 (m, 1H), 3.83-3.74 (m, 1H), 3.58 (s, 2H), 3.56 (d, J=4.0 Hz, 3H), 3.52-3.41 (m, 1H), 3.13-3.08 (m, 3H), 2.99-2.81 (m, 3H), 2.76-2.63 (m, 3H), 2.60-2.54 (m, 2H), 2.31-1.82 (m, 7H), 1.68-1.52 (m, 4H), 1.02 (d, J=6.8 Hz, 3H).

The compounds set forth in Table 21, were prepared using the synthetic procedures of Example 41, or analogous procedures as provided herein.

TABLE 21
Structure and Spectroscopic data for Compounds 780 to 822, 824 to 849, and 851 to 954

| No. | Structure |
|---|---|
| 780 | 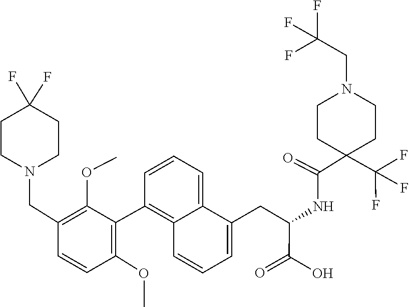 [SMILES: COc1ccc(CN2CCC(F)(F)CC2)c(OC)c1-c1cccc2c(C[C@H](NC(=O)C3(C(F)(F)F)CCN(CC(F)(F)F)CC3)C(=O)O)cccc12] |
| | m/z = 746.4 |
| 781 | 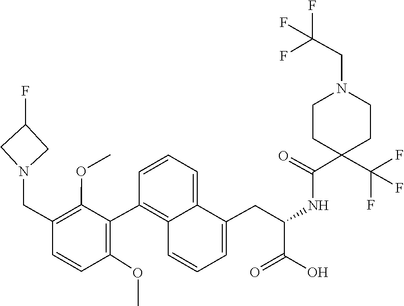 [SMILES: COc1ccc(CN2CC(F)C2)c(OC)c1-c1cccc2c(C[C@H](NC(=O)C3(C(F)(F)F)CCN(CC(F)(F)F)CC3)C(=O)O)cccc12] |
| | m/z = 700.2 |
| 782 | 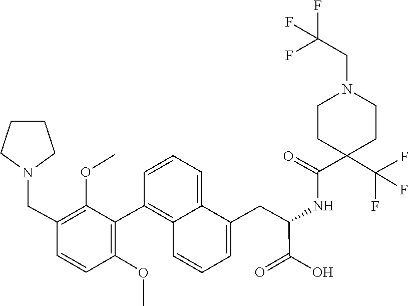 [SMILES: COc1ccc(CN2CCCC2)c(OC)c1-c1cccc2c(C[C@H](NC(=O)C3(C(F)(F)F)CCN(CC(F)(F)F)CC3)C(=O)O)cccc12] |
| | m/z = 696.1 |
| 783 | 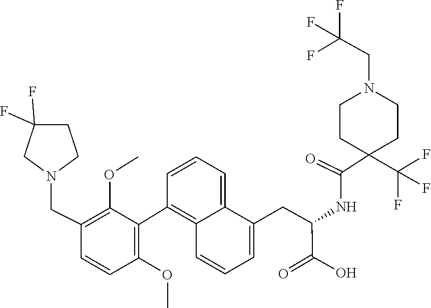 [SMILES: COc1ccc(CN2CCC(F)(F)C2)c(OC)c1-c1cccc2c(C[C@H](NC(=O)C3(C(F)(F)F)CCN(CC(F)(F)F)CC3)C(=O)O)cccc12] |
| | m/z = 732.3 |
| 784 | 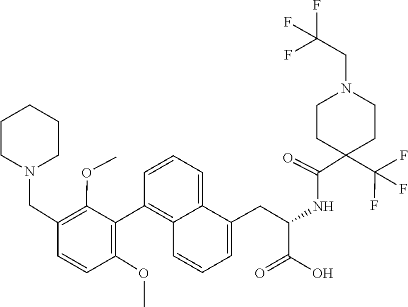 [SMILES: COc1ccc(CN2CCCCC2)c(OC)c1-c1cccc2c(C[C@H](NC(=O)C3(C(F)(F)F)CCN(CC(F)(F)F)CC3)C(=O)O)cccc12] |
| | m/z = 710.1 |
| 785 | 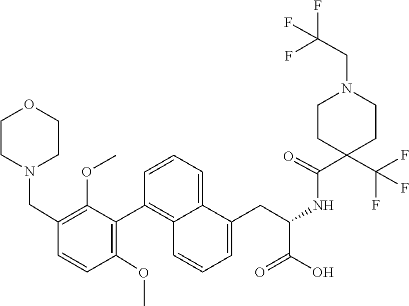 [SMILES: COc1ccc(CN2CCOCC2)c(OC)c1-c1cccc2c(C[C@H](NC(=O)C3(C(F)(F)F)CCN(CC(F)(F)F)CC3)C(=O)O)cccc12] |
| | m/z = 712.1 |
| 786 | 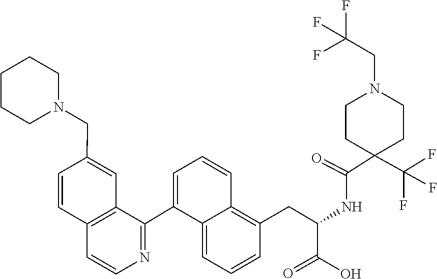 [SMILES: O=C(O)[C@H](Cc1cccc2c(-c3nccc4ccc(CN5CCCCC5)cc34)cccc12)NC(=O)C1(C(F)(F)F)CCN(CC(F)(F)F)CC1] |
| | m/z = 701.2 |
| 787 | 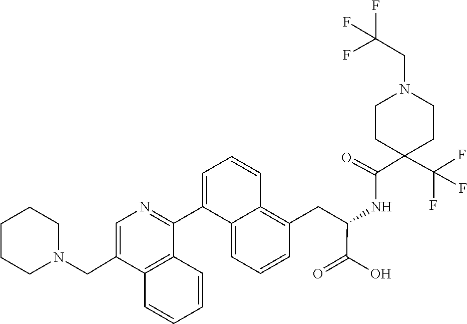 [SMILES: O=C(O)[C@H](Cc1cccc2c(-c3ncc(CN4CCCCC4)c4ccccc34)cccc12)NC(=O)C1(C(F)(F)F)CCN(CC(F)(F)F)CC1] |
| | m/z = 701.4 |
| 788* | 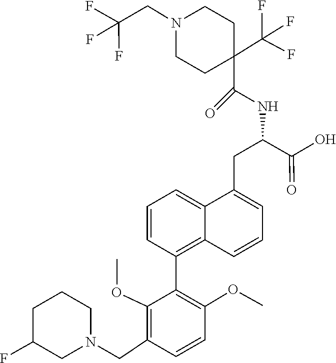 [SMILES: COc1ccc(CN2CCCC(F)C2)c(OC)c1-c1cccc2c(C[C@H](NC(=O)C3(C(F)(F)F)CCN(CC(F)(F)F)CC3)C(=O)O)cccc12] |
| | m/z = 728.4 |
| 789 | 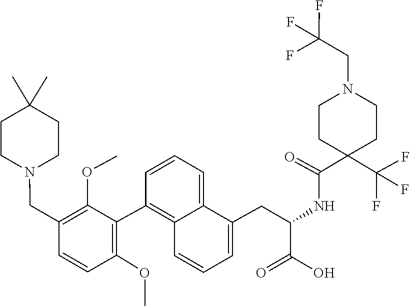 [SMILES: COc1ccc(CN2CCC(C)(C)CC2)c(OC)c1-c1cccc2c(C[C@H](NC(=O)C3(C(F)(F)F)CCN(CC(F)(F)F)CC3)C(=O)O)cccc12] |
| | m/z = 738.5 |
| 790* | 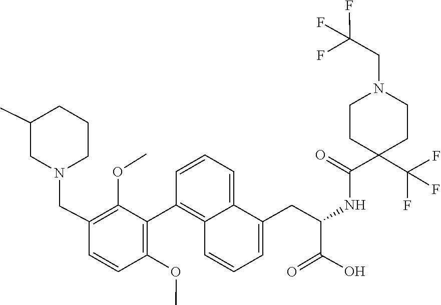 [SMILES: COc1ccc(CN2CCCC(C)C2)c(OC)c1-c1cccc2c(C[C@H](NC(=O)C3(C(F)(F)F)CCN(CC(F)(F)F)CC3)C(=O)O)cccc12] |
| | m/z = 724.3 |
| 791 | 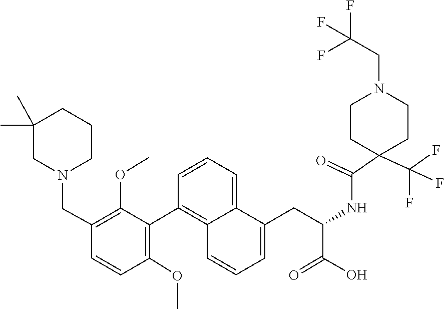 [SMILES: COc1ccc(CN2CCCC(C)(C)C2)c(OC)c1-c1cccc2c(C[C@H](NC(=O)C3(C(F)(F)F)CCN(CC(F)(F)F)CC3)C(=O)O)cccc12] |
| | m/z = 738.4 |
| 792 | 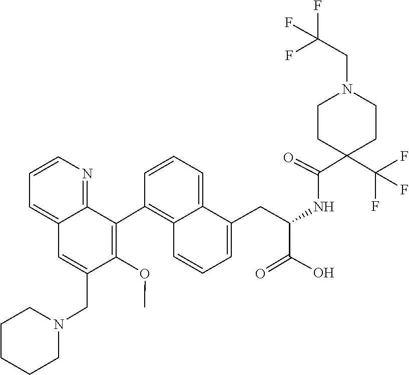 [SMILES: COc1c(CN2CCCCC2)cc2cccnc2c1-c1cccc2c(C[C@H](NC(=O)C3(C(F)(F)F)CCN(CC(F)(F)F)CC3)C(=O)O)cccc12] |
| | m/z = 731.2 |
| 793 | 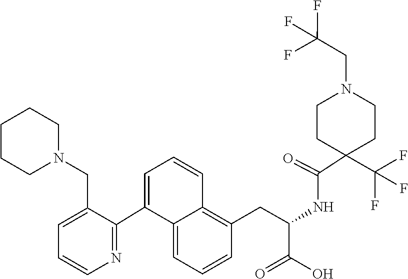 [SMILES: O=C(O)[C@H](Cc1cccc2c(-c3ncccc3CN3CCCCC3)cccc12)NC(=O)C1(C(F)(F)F)CCN(CC(F)(F)F)CC1] |
| | m/z = 651.3 |
| 794 | 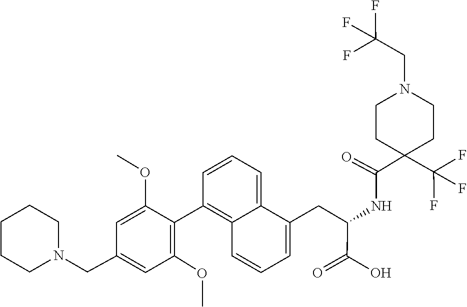 [SMILES: COc1cc(CN2CCCCC2)cc(OC)c1-c1cccc2c(C[C@H](NC(=O)C3(C(F)(F)F)CCN(CC(F)(F)F)CC3)C(=O)O)cccc12] |
| | m/z = 710.5 |
| 795 | 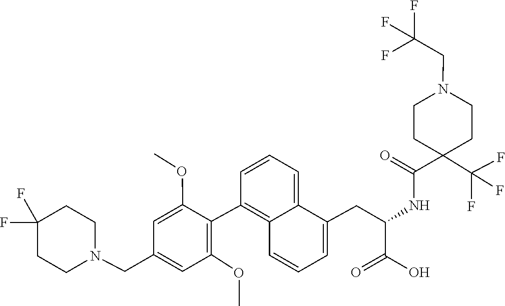 [SMILES: COc1cc(CN2CCC(F)(F)CC2)cc(OC)c1-c1cccc2c(C[C@H](NC(=O)C3(C(F)(F)F)CCN(CC(F)(F)F)CC3)C(=O)O)cccc12] |
| | m/z = 746.4 |
| 796 | 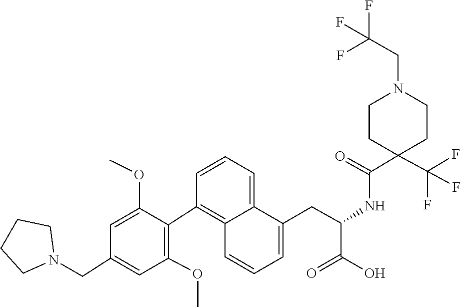 [SMILES: COc1cc(CN2CCCC2)cc(OC)c1-c1cccc2c(C[C@H](NC(=O)C3(C(F)(F)F)CCN(CC(F)(F)F)CC3)C(=O)O)cccc12] |
| | m/z = 696.5 |
| 797 | 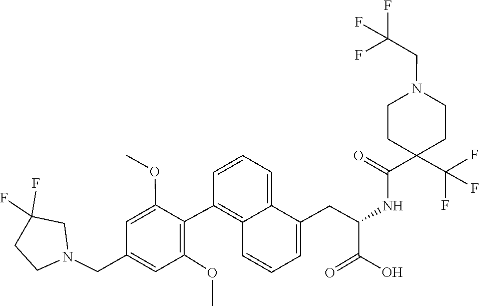 [SMILES: COc1cc(CN2CCC(F)(F)C2)cc(OC)c1-c1cccc2c(C[C@H](NC(=O)C3(C(F)(F)F)CCN(CC(F)(F)F)CC3)C(=O)O)cccc12] |
| | m/z = 732.4 |
| 798 | 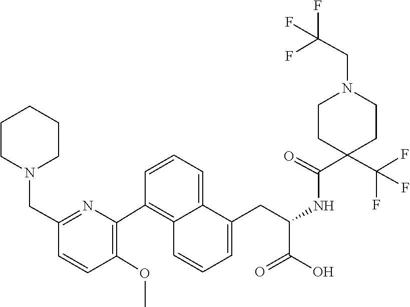 [SMILES: COc1ccc(CN2CCCCC2)nc1-c1cccc2c(C[C@H](NC(=O)C3(C(F)(F)F)CCN(CC(F)(F)F)CC3)C(=O)O)cccc12] |
| | m/z = 681.4 |
| 799 | 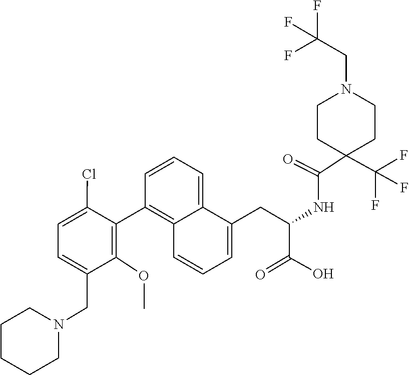 [SMILES: COc1c(CN2CCCCC2)ccc(Cl)c1-c1cccc2c(C[C@H](NC(=O)C3(C(F)(F)F)CCN(CC(F)(F)F)CC3)C(=O)O)cccc12] |
| | m/z = 714.2 |
| 800 | 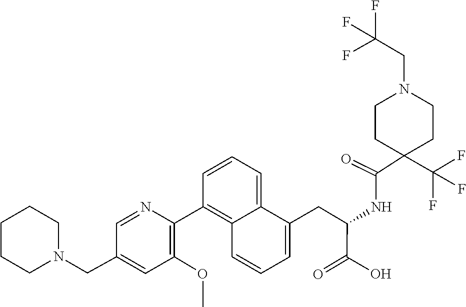 [SMILES: COc1cc(CN2CCCCC2)cnc1-c1cccc2c(C[C@H](NC(=O)C3(C(F)(F)F)CCN(CC(F)(F)F)CC3)C(=O)O)cccc12] |
| | m/z = 681.4 |
| 801 | 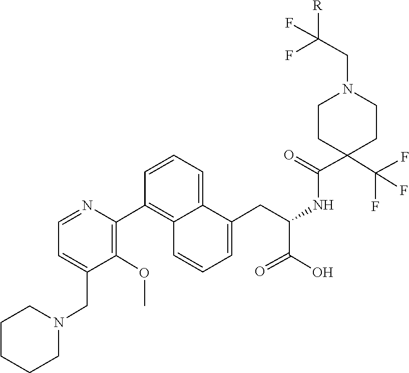 |
| | m/z = 681.2 |
| 802 | 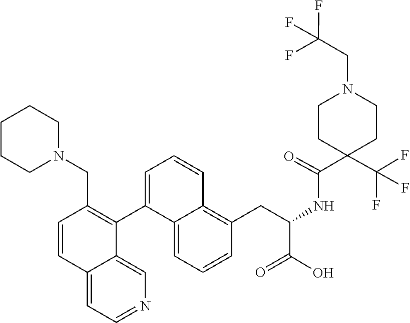 [SMILES: O=C(O)[C@H](Cc1cccc2c(-c3c(CN4CCCCC4)ccc4ccncc34)cccc12)NC(=O)C1(C(F)(F)F)CCN(CC(F)(F)F)CC1] |
| | m/z = 701.2 |
| 803 | 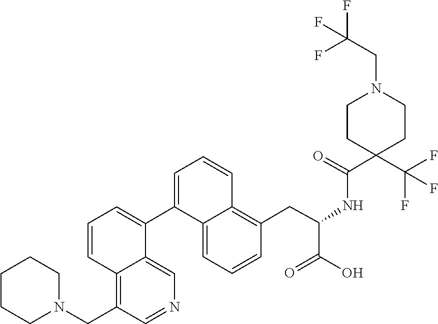 [SMILES: O=C(O)[C@H](Cc1cccc2c(-c3cccc4c(CN5CCCCC5)cncc34)cccc12)NC(=O)C1(C(F)(F)F)CCN(CC(F)(F)F)CC1] |
| | m/z = 701.4 |
| 804 | 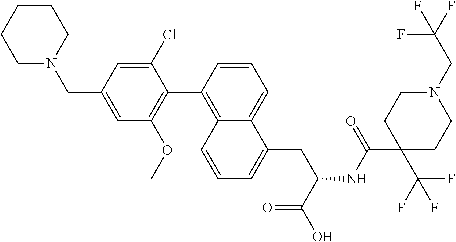 [SMILES: COc1cc(CN2CCCCC2)cc(Cl)c1-c1cccc2c(C[C@H](NC(=O)C3(C(F)(F)F)CCN(CC(F)(F)F)CC3)C(=O)O)cccc12] |
| | m/z = 714.2 |
| 805 | 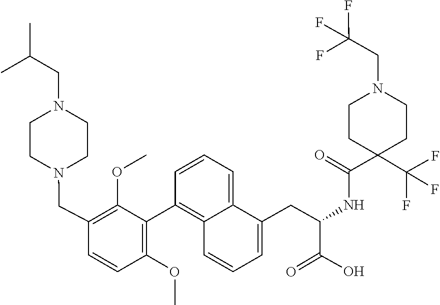 [SMILES: COc1ccc(CN2CCN(CC(C)C)CC2)c(OC)c1-c1cccc2c(C[C@H](NC(=O)C3(C(F)(F)F)CCN(CC(F)(F)F)CC3)C(=O)O)cccc12] |
| | m/z = 767.5 |
| 806 | 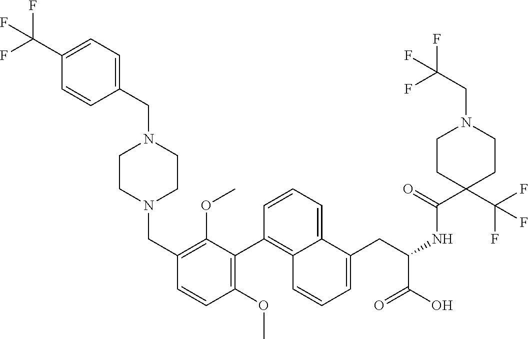 [SMILES: COc1ccc(CN2CCN(Cc3ccc(C(F)(F)F)cc3)CC2)c(OC)c1-c1cccc2c(C[C@H](NC(=O)C3(C(F)(F)F)CCN(CC(F)(F)F)CC3)C(=O)O)cccc12] |
| | m/z = 869.2 |
| 807 | 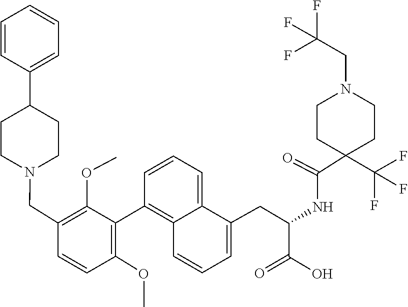 [SMILES: COc1ccc(CN2CCC(c3ccccc3)CC2)c(OC)c1-c1cccc2c(C[C@H](NC(=O)C3(C(F)(F)F)CCN(CC(F)(F)F)CC3)C(=O)O)cccc12] |
| | m/z = 786.5 |
| 808 | 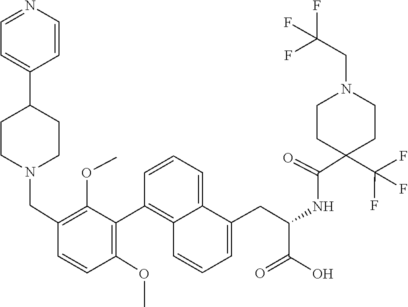 [SMILES: COc1ccc(CN2CCC(c3ccncc3)CC2)c(OC)c1-c1cccc2c(C[C@H](NC(=O)C3(C(F)(F)F)CCN(CC(F)(F)F)CC3)C(=O)O)cccc12] |
| | m/z = 787.3 |
| 809 | 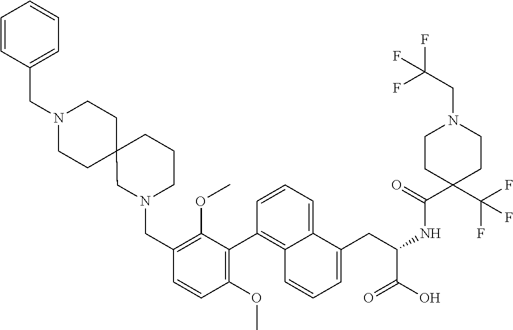 [SMILES: COc1ccc(CN2CCCC3(CCN(Cc4ccccc4)CC3)C2)c(OC)c1-c1cccc2c(C[C@H](NC(=O)C3(C(F)(F)F)CCN(CC(F)(F)F)CC3)C(=O)O)cccc12] |
| | m/z = 869.4 |
| 810 | 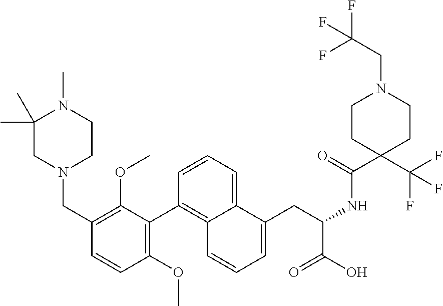 [SMILES: COc1ccc(CN2CCN(C)C(C)(C)C2)c(OC)c1-c1cccc2c(C[C@H](NC(=O)C3(C(F)(F)F)CCN(CC(F)(F)F)CC3)C(=O)O)cccc12] |
| | m/z = 753.2 |
| 811 | 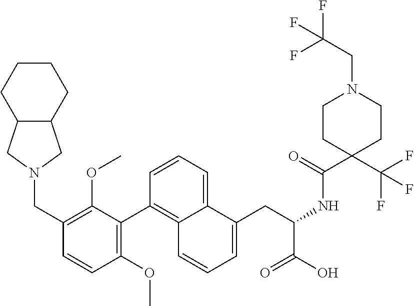 [SMILES: COc1ccc(CN2CC3CCCCC3C2)c(OC)c1-c1cccc2c(C[C@H](NC(=O)C3(C(F)(F)F)CCN(CC(F)(F)F)CC3)C(=O)O)cccc12] |
| | m/z = 750.5 |
| 812 | 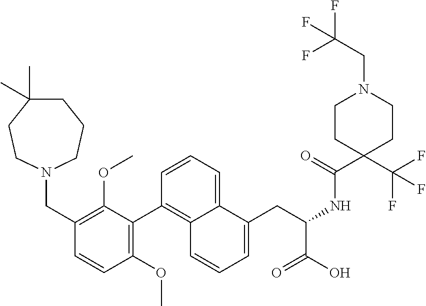 [SMILES: COc1ccc(CN2CCCC(C)(C)CC2)c(OC)c1-c1cccc2c(C[C@H](NC(=O)C3(C(F)(F)F)CCN(CC(F)(F)F)CC3)C(=O)O)cccc12] |
| | m/z = 752.5 |
| 813 | 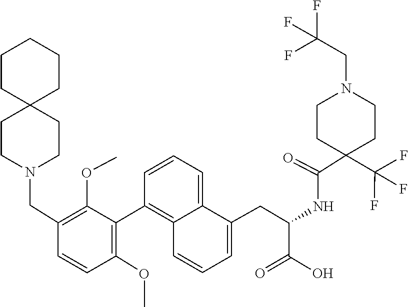 [SMILES: COc1ccc(CN2CCC3(CCCCC3)CC2)c(OC)c1-c1cccc2c(C[C@H](NC(=O)C3(C(F)(F)F)CCN(CC(F)(F)F)CC3)C(=O)O)cccc12] |
| | m/z = 778.2 |
| 814 | 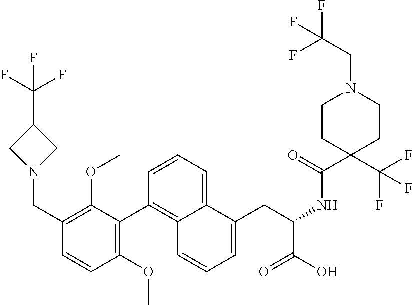 [SMILES: COc1ccc(CN2CC(C(F)(F)F)C2)c(OC)c1-c1cccc2c(C[C@H](NC(=O)C3(C(F)(F)F)CCN(CC(F)(F)F)CC3)C(=O)O)cccc12] |
| | m/z = 750.6 |
| 815 | 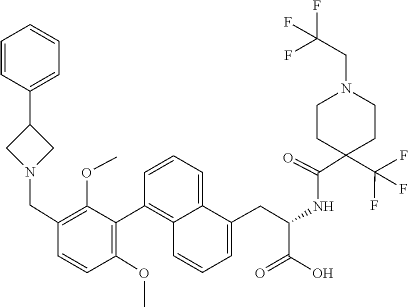 [SMILES: COc1ccc(CN2CC(c3ccccc3)C2)c(OC)c1-c1cccc2c(C[C@H](NC(=O)C3(C(F)(F)F)CCN(CC(F)(F)F)CC3)C(=O)O)cccc12] |
| | m/z = 758.4 |
| 816 | 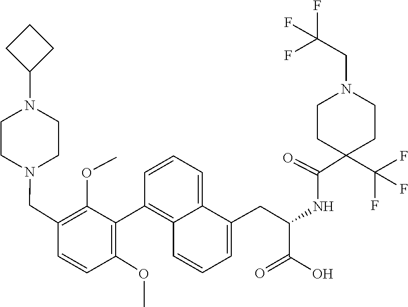 [SMILES: COc1ccc(CN2CCN(C3CCC3)CC2)c(OC)c1-c1cccc2c(C[C@H](NC(=O)C3(C(F)(F)F)CCN(CC(F)(F)F)CC3)C(=O)O)cccc12] |
| | m/z = 765.2 |
| 817* | 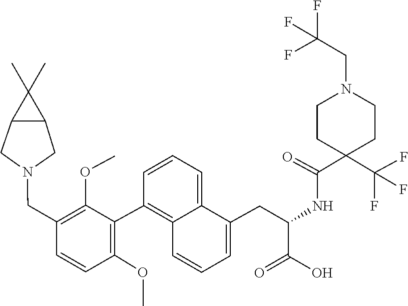 [SMILES: COc1ccc(CN2CC3C(C2)C3(C)C)c(OC)c1-c1cccc2c(C[C@H](NC(=O)C3(C(F)(F)F)CCN(CC(F)(F)F)CC3)C(=O)O)cccc12] |
| | m/z = 736.4 |
| 818 | 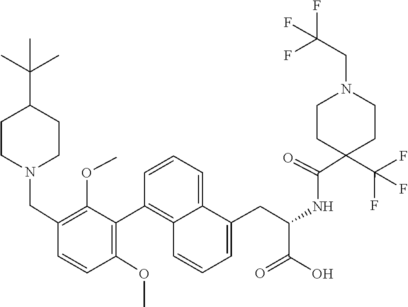 [SMILES: COc1ccc(CN2CCC(C(C)(C)C)CC2)c(OC)c1-c1cccc2c(C[C@H](NC(=O)C3(C(F)(F)F)CCN(CC(F)(F)F)CC3)C(=O)O)cccc12] |
| | m/z = 766.6 |
| 819 | 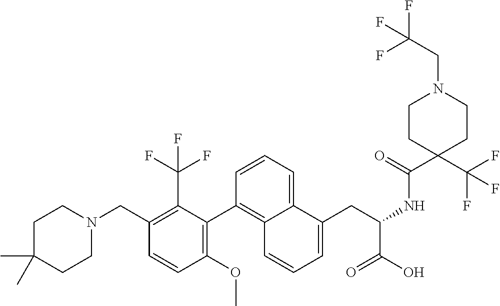 [SMILES: COc1ccc(CN2CCC(C)(C)CC2)c(C(F)(F)F)c1-c1cccc2c(C[C@H](NC(=O)C3(C(F)(F)F)CCN(CC(F)(F)F)CC3)C(=O)O)cccc12] |
| | m/z = 776.5 |
| 820* | 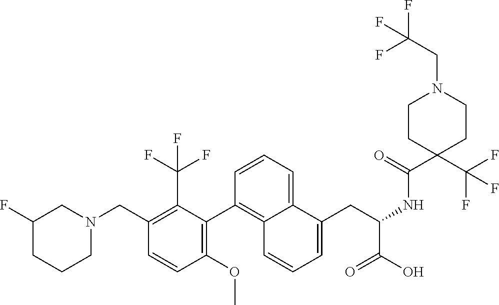 [SMILES: COc1ccc(CN2CCCC(F)C2)c(C(F)(F)F)c1-c1cccc2c(C[C@H](NC(=O)C3(C(F)(F)F)CCN(CC(F)(F)F)CC3)C(=O)O)cccc12] |
| | m/z = 766.4 |
| 821 | 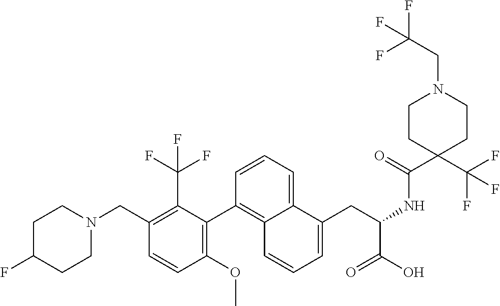 [SMILES: COc1ccc(CN2CCC(F)CC2)c(C(F)(F)F)c1-c1cccc2c(C[C@H](NC(=O)C3(C(F)(F)F)CCN(CC(F)(F)F)CC3)C(=O)O)cccc12] |
| | m/z = 766.4 |
| 822 | 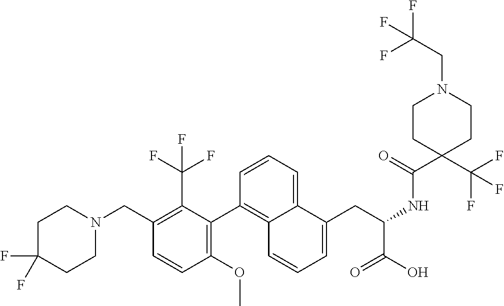 [SMILES: COc1ccc(CN2CCC(F)(F)CC2)c(C(F)(F)F)c1-c1cccc2c(C[C@H](NC(=O)C3(C(F)(F)F)CCN(CC(F)(F)F)CC3)C(=O)O)cccc12] |
| | m/z = 784.3 |
| 824 | 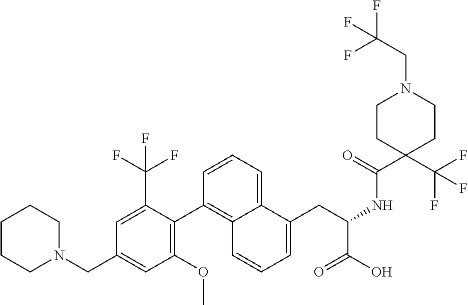 [SMILES: COc1cc(CN2CCCCC2)cc(C(F)(F)F)c1-c1cccc2c(C[C@H](NC(=O)C3(C(F)(F)F)CCN(CC(F)(F)F)CC3)C(=O)O)cccc12] |
| | m/z = 748.4 |
| 825 | 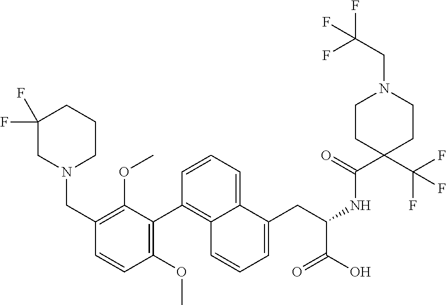 [SMILES: COc1ccc(CN2CCCC(F)(F)C2)c(OC)c1-c1cccc2c(C[C@H](NC(=O)C3(C(F)(F)F)CCN(CC(F)(F)F)CC3)C(=O)O)cccc12] |
| | m/z = 746.1 |
| 826* | 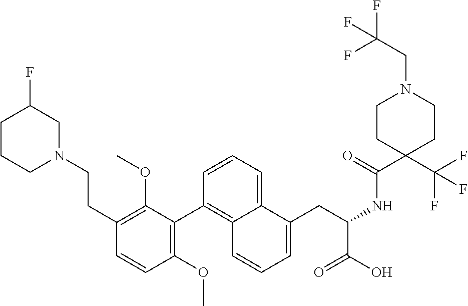 [SMILES: COc1ccc(CCN2CCCC(F)C2)c(OC)c1-c1cccc2c(C[C@H](NC(=O)C3(C(F)(F)F)CCN(CC(F)(F)F)CC3)C(=O)O)cccc12] |
| | m/z = 742.3 |
| 827 | 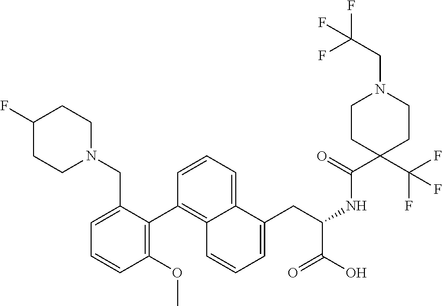 [SMILES: COc1cccc(CN2CCC(F)CC2)c1-c1cccc2c(C[C@H](NC(=O)C3(C(F)(F)F)CCN(CC(F)(F)F)CC3)C(=O)O)cccc12] |
| | m/z = 698.1 |
| 828* | 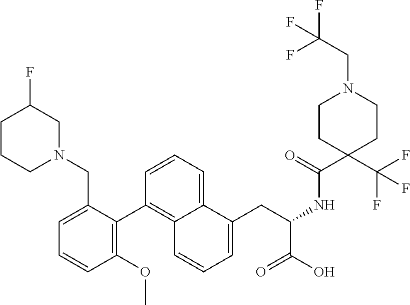 [SMILES: COc1cccc(CN2CCCC(F)C2)c1-c1cccc2c(C[C@H](NC(=O)C3(C(F)(F)F)CCN(CC(F)(F)F)CC3)C(=O)O)cccc12] |
| | m/z = 698.3 |
| 829 | 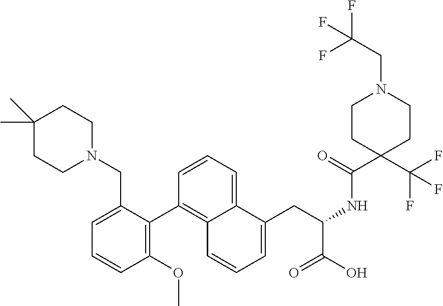 [SMILES: COc1cccc(CN2CCC(C)(C)CC2)c1-c1cccc2c(C[C@H](NC(=O)C3(C(F)(F)F)CCN(CC(F)(F)F)CC3)C(=O)O)cccc12] |
| | m/z = 708.2 |
| 830 | 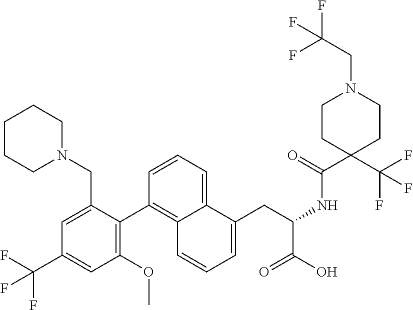 [SMILES: COc1cc(C(F)(F)F)cc(CN2CCCCC2)c1-c1cccc2c(C[C@H](NC(=O)C3(C(F)(F)F)CCN(CC(F)(F)F)CC3)C(=O)O)cccc12] |
| | m/z = 748.4 |
| 831 | 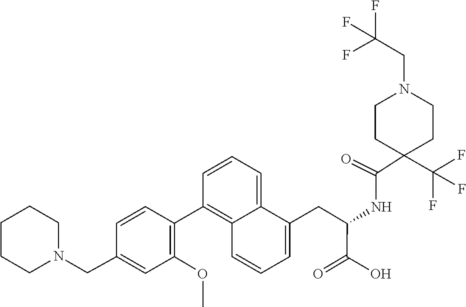 [SMILES: COc1cc(CN2CCCCC2)ccc1-c1cccc2c(C[C@H](NC(=O)C3(C(F)(F)F)CCN(CC(F)(F)F)CC3)C(=O)O)cccc12] |
| | m/z = 680.1 |
| 832 | 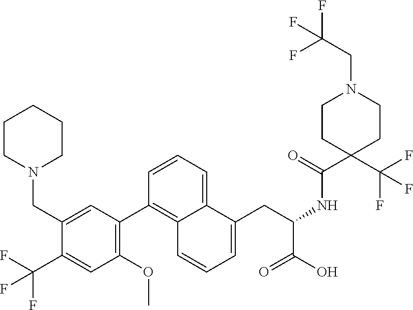 [SMILES: COc1cc(C(F)(F)F)c(CN2CCCCC2)cc1-c1cccc2c(C[C@H](NC(=O)C3(C(F)(F)F)CCN(CC(F)(F)F)CC3)C(=O)O)cccc12] |
| | m/z = 748.1 |
| 833 | 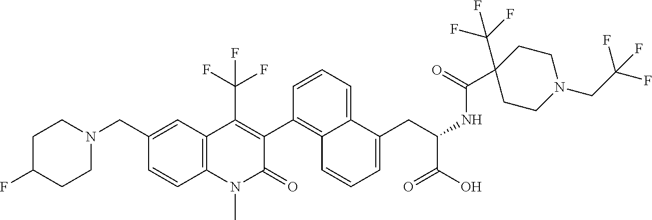 [SMILES: Cn1c(=O)c(-c2cccc3c(C[C@H](NC(=O)C4(C(F)(F)F)CCN(CC(F)(F)F)CC4)C(=O)O)cccc23)c(C(F)(F)F)c2cc(CN3CCC(F)CC3)ccc21] |
| | m/z = 817.2 |
| 834 | 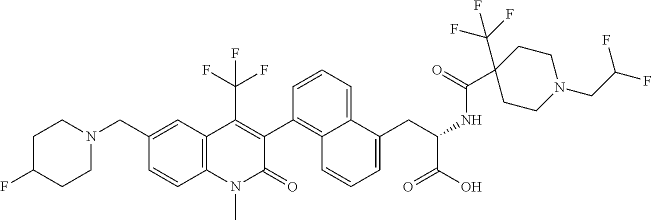 [SMILES: Cn1c(=O)c(-c2cccc3c(C[C@H](NC(=O)C4(C(F)(F)F)CCN(CC(F)F)CC4)C(=O)O)cccc23)c(C(F)(F)F)c2cc(CN3CCC(F)CC3)ccc21] |
| | m/z = 799.2 |
| 835 | 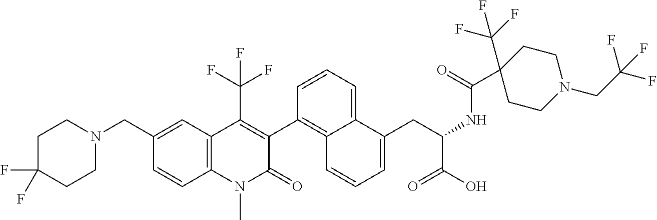 [SMILES: Cn1c(=O)c(-c2cccc3c(C[C@H](NC(=O)C4(C(F)(F)F)CCN(CC(F)(F)F)CC4)C(=O)O)cccc23)c(C(F)(F)F)c2cc(CN3CCC(F)(F)CC3)ccc21] |
| | m/z = 835.5 |
| 836 | 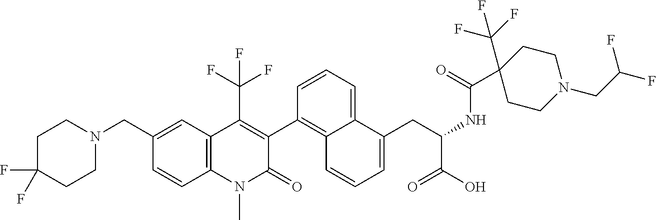 [SMILES: Cn1c(=O)c(-c2cccc3c(C[C@H](NC(=O)C4(C(F)(F)F)CCN(CC(F)F)CC4)C(=O)O)cccc23)c(C(F)(F)F)c2cc(CN3CCC(F)(F)CC3)ccc21] |
| | m/z = 817.1 |
| 837 | 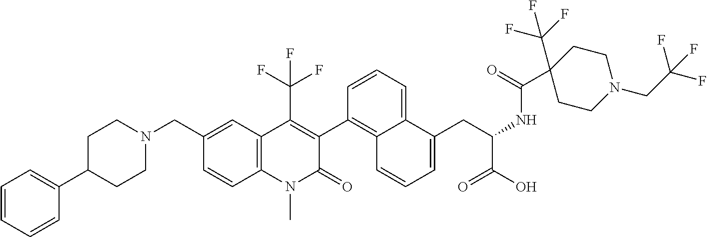 [SMILES: Cn1c(=O)c(-c2cccc3c(C[C@H](NC(=O)C4(C(F)(F)F)CCN(CC(F)(F)F)CC4)C(=O)O)cccc23)c(C(F)(F)F)c2cc(CN3CCC(c4ccccc4)CC3)ccc21] |
| | m/z = 875.3 |
| 838 | 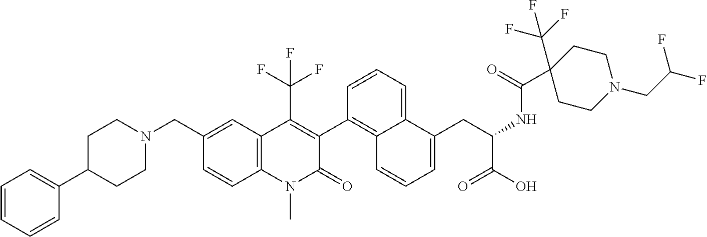 [SMILES: Cn1c(=O)c(-c2cccc3c(C[C@H](NC(=O)C4(C(F)(F)F)CCN(CC(F)F)CC4)C(=O)O)cccc23)c(C(F)(F)F)c2cc(CN3CCC(c4ccccc4)CC3)ccc21] |
| | m/z = 857.2 |
| 839* | 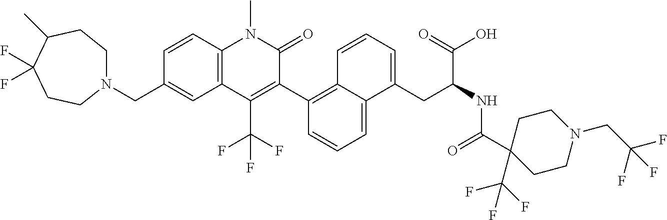 [SMILES: CC1CCN(Cc2ccc3c(c2)c(C(F)(F)F)c(-c2cccc4c(C[C@H](NC(=O)C5(C(F)(F)F)CCN(CC(F)(F)F)CC5)C(=O)O)cccc24)c(=O)n3C)CCC1(F)F] |
| | m/z = 863.2 |
| 840* | 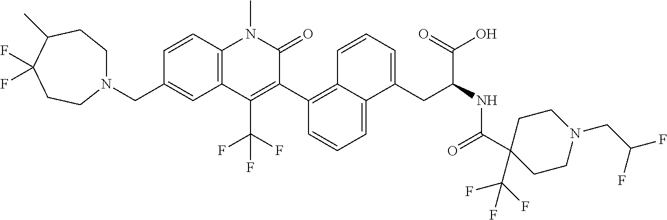 [SMILES: CC1CCN(Cc2ccc3c(c2)c(C(F)(F)F)c(-c2cccc4c(C[C@H](NC(=O)C5(C(F)(F)F)CCN(CC(F)F)CC5)C(=O)O)cccc24)c(=O)n3C)CCC1(F)F] |
| | m/z = 845.2 |
| 841 | 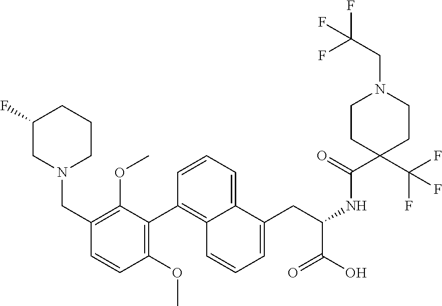 [SMILES: COc1ccc(CN2CCC[C@@H](F)C2)c(OC)c1-c1cccc2c(C[C@H](NC(=O)C3(C(F)(F)F)CCN(CC(F)(F)F)CC3)C(=O)O)cccc12] |
| | m/z = 728.3 |
| 842 | 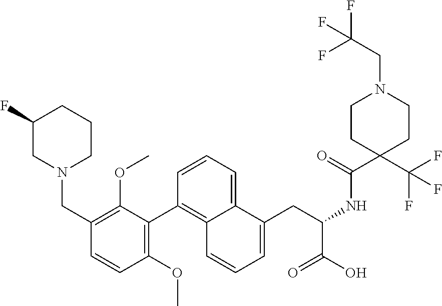 [SMILES: COc1ccc(CN2CCC[C@H](F)C2)c(OC)c1-c1cccc2c(C[C@H](NC(=O)C3(C(F)(F)F)CCN(CC(F)(F)F)CC3)C(=O)O)cccc12] |
| | m/z = 728.3 |
| 843* | 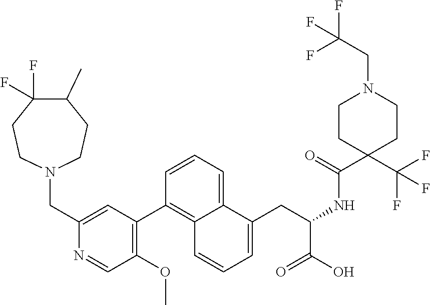 [SMILES: COc1cnc(CN2CCC(C)C(F)(F)CC2)cc1-c1cccc2c(C[C@H](NC(=O)C3(C(F)(F)F)CCN(CC(F)(F)F)CC3)C(=O)O)cccc12] |
| | m/z = 745.5 |
| 844 | 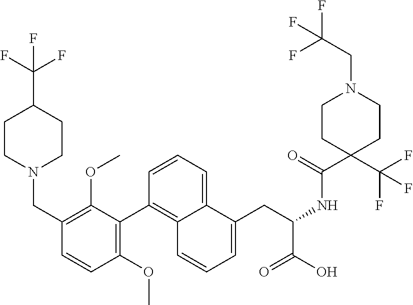 [SMILES: COc1ccc(CN2CCC(C(F)(F)F)CC2)c(OC)c1-c1cccc2c(C[C@H](NC(=O)C3(C(F)(F)F)CCN(CC(F)(F)F)CC3)C(=O)O)cccc12] |
| | m/z = 778.4 |
| 845 | 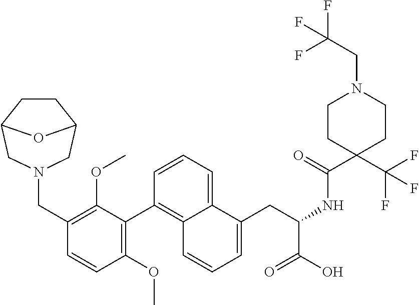 [SMILES: COc1ccc(CN2CC3CCC(C2)O3)c(OC)c1-c1cccc2c(C[C@H](NC(=O)C3(C(F)(F)F)CCN(CC(F)(F)F)CC3)C(=O)O)cccc12] |
| | m/z = 738.3 |
| 846 | 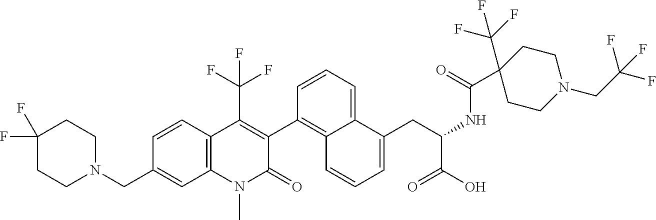 [SMILES: Cn1c(=O)c(-c2cccc3c(C[C@H](NC(=O)C4(C(F)(F)F)CCN(CC(F)(F)F)CC4)C(=O)O)cccc23)c(C(F)(F)F)c2ccc(CN3CCC(F)(F)CC3)cc21] |
| | m/z = 835.3 |
| 847 | 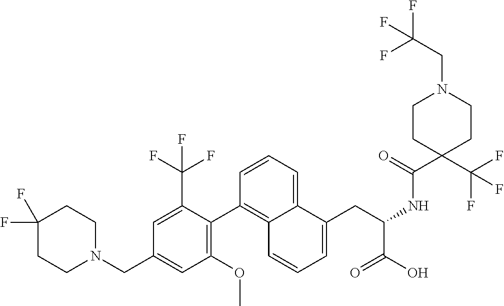 [SMILES: COc1cc(CN2CCC(F)(F)CC2)cc(C(F)(F)F)c1-c1cccc2c(C[C@H](NC(=O)C3(C(F)(F)F)CCN(CC(F)(F)F)CC3)C(=O)O)cccc12] |
| | m/z = 784.4 |
| 848 | 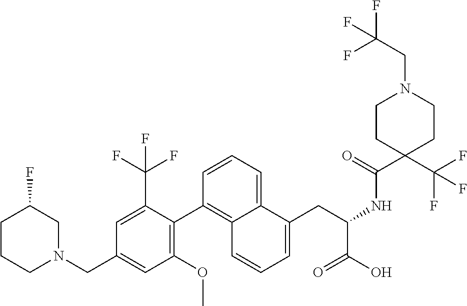 [SMILES: COc1cc(CN2CCC[C@H](F)C2)cc(C(F)(F)F)c1-c1cccc2c(C[C@H](NC(=O)C3(C(F)(F)F)CCN(CC(F)(F)F)CC3)C(=O)O)cccc12] |
| | m/z = 766.2 |
| 849 | 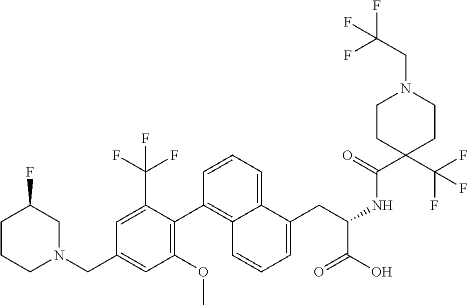 [SMILES: COc1cc(CN2CCC[C@@H](F)C2)cc(C(F)(F)F)c1-c1cccc2c(C[C@H](NC(=O)C3(C(F)(F)F)CCN(CC(F)(F)F)CC3)C(=O)O)cccc12] |
| | m/z = 766.5 |
| 851 | 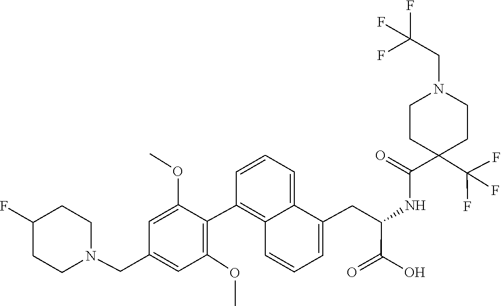 [SMILES: COc1cc(CN2CCC(F)CC2)cc(OC)c1-c1cccc2c(C[C@H](NC(=O)C3(C(F)(F)F)CCN(CC(F)(F)F)CC3)C(=O)O)cccc12] |
| | m/z = 728.3 |
| 852 | 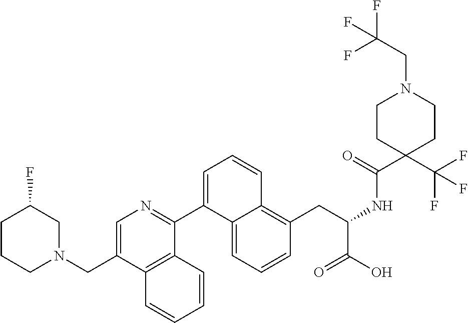 [SMILES: O=C(O)[C@H](Cc1cccc2c(-c3ncc(CN4CCC[C@H](F)C4)c4ccccc34)cccc12)NC(=O)C1(C(F)(F)F)CCN(CC(F)(F)F)CC1] |
| | m/z = 719.3 |
| 853 | 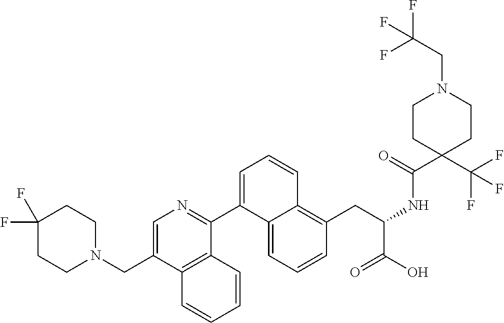 [SMILES: O=C(O)[C@H](Cc1cccc2c(-c3ncc(CN4CCC(F)(F)CC4)c4ccccc34)cccc12)NC(=O)C1(C(F)(F)F)CCN(CC(F)(F)F)CC1] |
| | m/z = 737.2 |
| 854 | 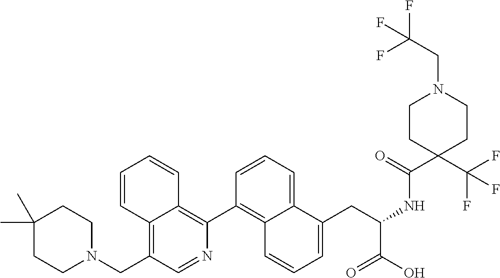 [SMILES: CC1(C)CCN(Cc2cnc(-c3cccc4c(C[C@H](NC(=O)C5(C(F)(F)F)CCN(CC(F)(F)F)CC5)C(=O)O)cccc34)c3ccccc23)CC1] |
| | m/z = 729.3 |
| 855 | 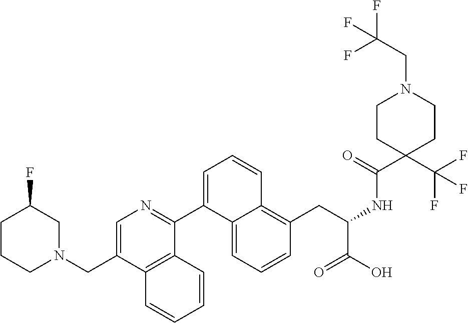 [SMILES: O=C(O)[C@H](Cc1cccc2c(-c3ncc(CN4CCC[C@@H](F)C4)c4ccccc34)cccc12)NC(=O)C1(C(F)(F)F)CCN(CC(F)(F)F)CC1] |
| | m/z = 719.4 |
| 856* | 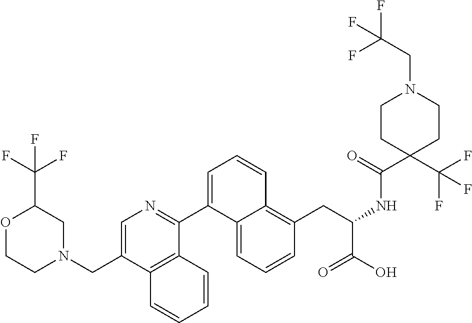 [SMILES: O=C(O)[C@H](Cc1cccc2c(-c3ncc(CN4CCOC(C(F)(F)F)C4)c4ccccc34)cccc12)NC(=O)C1(C(F)(F)F)CCN(CC(F)(F)F)CC1] |
| | m/z = 771.2 |
| 857 | 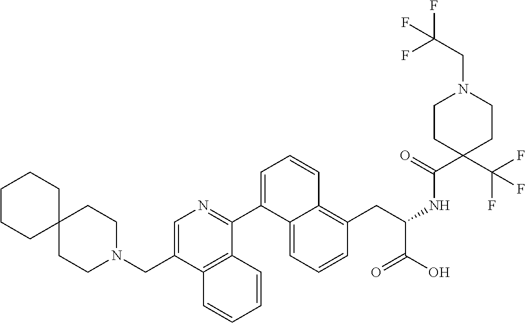 [SMILES: O=C(O)[C@H](Cc1cccc2c(-c3ncc(CN4CCC5(CCCCC5)CC4)c4ccccc34)cccc12)NC(=O)C1(C(F)(F)F)CCN(CC(F)(F)F)CC1] |
| | m/z = 769.4 |
| 858* | 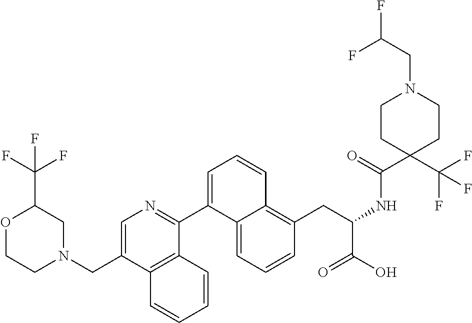 [SMILES: O=C(O)[C@H](Cc1cccc2c(-c3ncc(CN4CCOC(C(F)(F)F)C4)c4ccccc34)cccc12)NC(=O)C1(C(F)(F)F)CCN(CC(F)F)CC1] |
| | m/z = 753.2 |
| 859 | 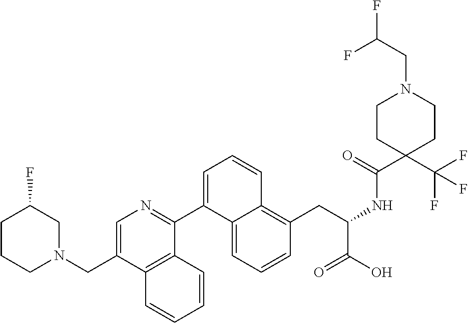 [SMILES: O=C(O)[C@H](Cc1cccc2c(-c3ncc(CN4CCC[C@H](F)C4)c4ccccc34)cccc12)NC(=O)C1(C(F)(F)F)CCN(CC(F)F)CC1] |
| | m/z = 701.1 |
| 860 | 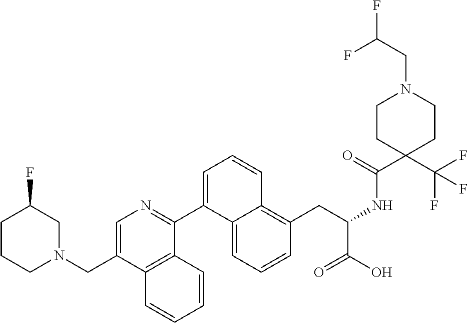 [SMILES: O=C(O)[C@H](Cc1cccc2c(-c3ncc(CN4CCC[C@@H](F)C4)c4ccccc34)cccc12)NC(=O)C1(C(F)(F)F)CCN(CC(F)F)CC1] |
| | m/z = 701.5 |
| 861 | 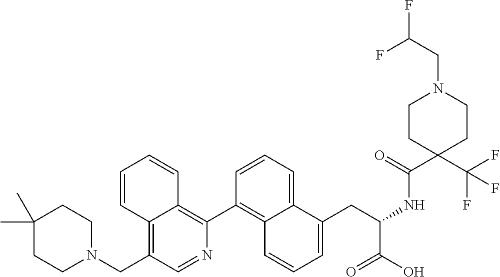 [SMILES: CC1(C)CCN(Cc2cnc(-c3cccc4c(C[C@H](NC(=O)C5(C(F)(F)F)CCN(CC(F)F)CC5)C(=O)O)cccc34)c3ccccc23)CC1] |
| | m/z = 711.2 |
| 862 | 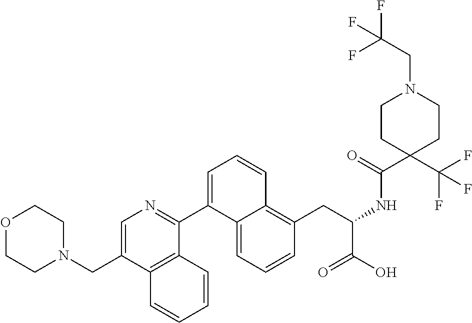 [SMILES: O=C(O)[C@H](Cc1cccc2c(-c3ncc(CN4CCOCC4)c4ccccc34)cccc12)NC(=O)C1(C(F)(F)F)CCN(CC(F)(F)F)CC1] |
| | m/z = 703.1 |
| 863 | 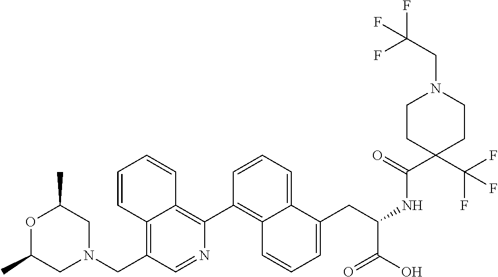 [SMILES: C[C@@H]1CN(Cc2cnc(-c3cccc4c(C[C@H](NC(=O)C5(C(F)(F)F)CCN(CC(F)(F)F)CC5)C(=O)O)cccc34)c3ccccc23)C[C@H](C)O1] |
| | m/z = 731.4 |
| 864 | 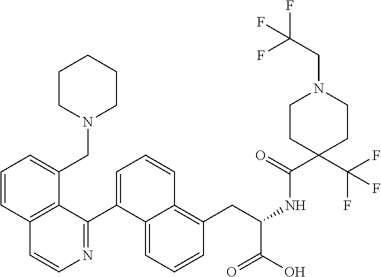 [SMILES: O=C(O)[C@H](Cc1cccc2c(-c3nccc4cccc(CN5CCCCC5)c34)cccc12)NC(=O)C1(C(F)(F)F)CCN(CC(F)(F)F)CC1] |
| | m/z = 701.3 |
| 865 | 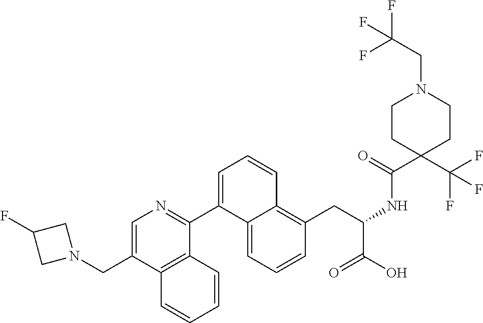 [SMILES: O=C(O)[C@H](Cc1cccc2c(-c3ncc(CN4CC(F)C4)c4ccccc34)cccc12)NC(=O)C1(C(F)(F)F)CCN(CC(F)(F)F)CC1] |
| | m/z = 691.3 |
| 866 | 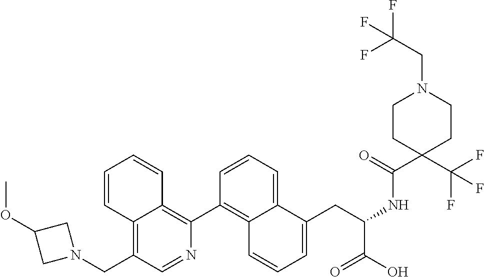 [SMILES: COC1CN(Cc2cnc(-c3cccc4c(C[C@H](NC(=O)C5(C(F)(F)F)CCN(CC(F)(F)F)CC5)C(=O)O)cccc34)c3ccccc23)C1] |
| | m/z = 703.2 |
| 867 | 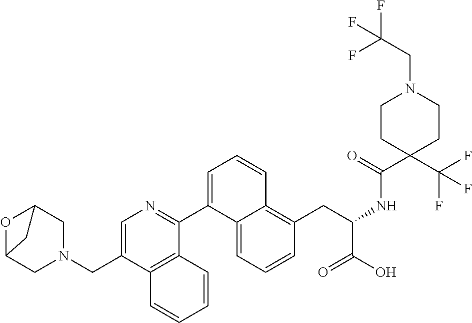 [SMILES: O=C(O)[C@H](Cc1cccc2c(-c3ncc(CN4CC5CC(C4)O5)c4ccccc34)cccc12)NC(=O)C1(C(F)(F)F)CCN(CC(F)(F)F)CC1] |
| | m/z = 715.4 |
| 868 | 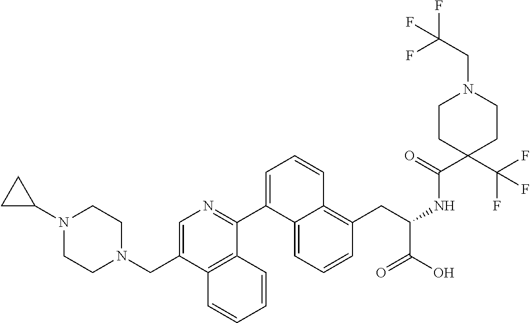 [SMILES: O=C(O)[C@H](Cc1cccc2c(-c3ncc(CN4CCN(C5CC5)CC4)c4ccccc34)cccc12)NC(=O)C1(C(F)(F)F)CCN(CC(F)(F)F)CC1] |
| | m/z = 742.4 |
| 869 | 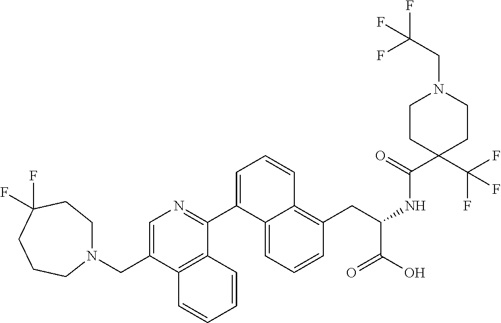 [SMILES: O=C(O)[C@H](Cc1cccc2c(-c3ncc(CN4CCCC(F)(F)CC4)c4ccccc34)cccc12)NC(=O)C1(C(F)(F)F)CCN(CC(F)(F)F)CC1] |
| | m/z = 751.4 |
| 870 | 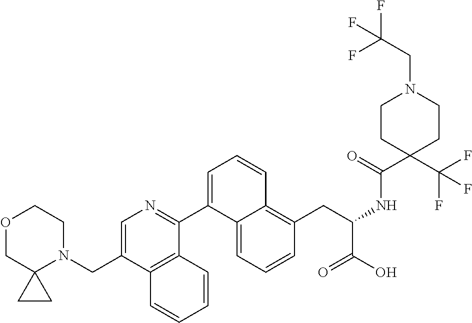 [SMILES: O=C(O)[C@H](Cc1cccc2c(-c3ncc(CN4CCOCC45CC5)c4ccccc34)cccc12)NC(=O)C1(C(F)(F)F)CCN(CC(F)(F)F)CC1] |
| | m/z = 729.1 |
| 871 | 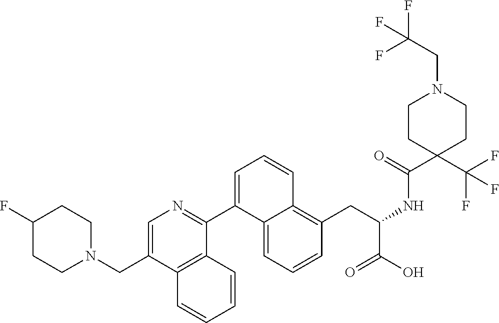 [SMILES: O=C(O)[C@H](Cc1cccc2c(-c3ncc(CN4CCC(F)CC4)c4ccccc34)cccc12)NC(=O)C1(C(F)(F)F)CCN(CC(F)(F)F)CC1] |
| | m/z = 719.1 |
| 872 | 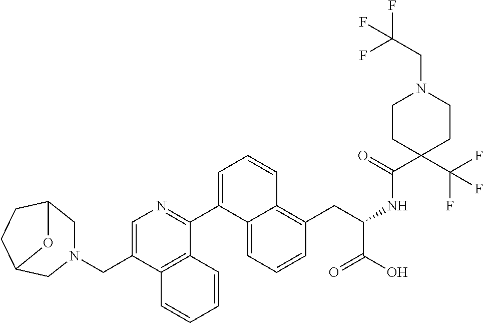 [SMILES: O=C(O)[C@H](Cc1cccc2c(-c3ncc(CN4CC5CCC(C4)O5)c4ccccc34)cccc12)NC(=O)C1(C(F)(F)F)CCN(CC(F)(F)F)CC1] |
| | m/z = 729.3 |
| 873 | 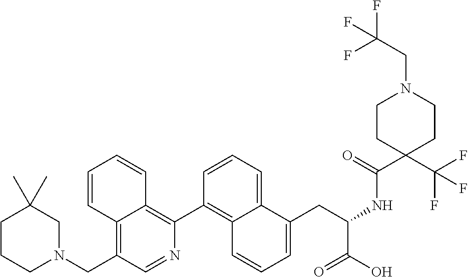 [SMILES: CC1(C)CCCN(Cc2cnc(-c3cccc4c(C[C@H](NC(=O)C5(C(F)(F)F)CCN(CC(F)(F)F)CC5)C(=O)O)cccc34)c3ccccc23)C1] |
| | m/z = 729.3 |
| 874 | 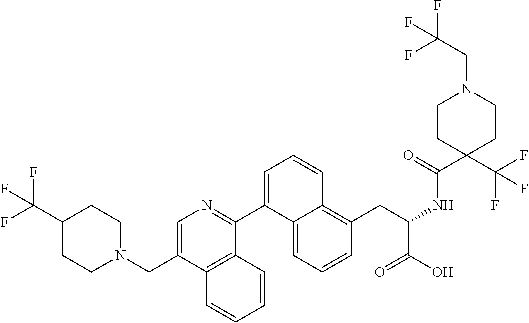 [SMILES: O=C(O)[C@H](Cc1cccc2c(-c3ncc(CN4CCC(C(F)(F)F)CC4)c4ccccc34)cccc12)NC(=O)C1(C(F)(F)F)CCN(CC(F)(F)F)CC1] |
| | m/z = 769.2 |
| 875* | 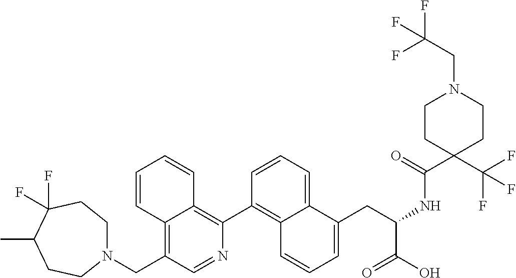 [SMILES: CC1CCN(Cc2cnc(-c3cccc4c(C[C@H](NC(=O)C5(C(F)(F)F)CCN(CC(F)(F)F)CC5)C(=O)O)cccc34)c3ccccc23)CCC1(F)F] |
| | m/z = 765.4 |
| 876 | 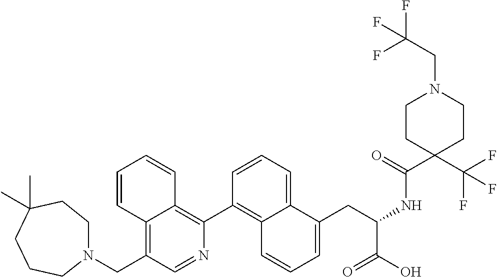 [SMILES: CC1(C)CCCN(Cc2cnc(-c3cccc4c(C[C@H](NC(=O)C5(C(F)(F)F)CCN(CC(F)(F)F)CC5)C(=O)O)cccc34)c3ccccc23)CC1] |
| | m/z = 743.4 |
| 877 | 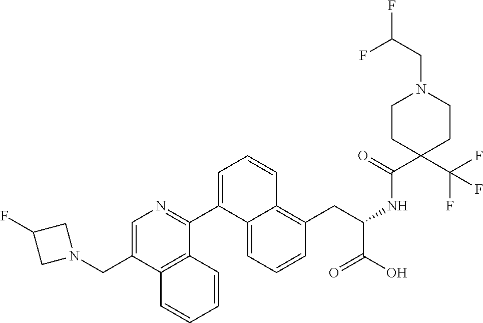 [SMILES: O=C(O)[C@H](Cc1cccc2c(-c3ncc(CN4CC(F)C4)c4ccccc34)cccc12)NC(=O)C1(C(F)(F)F)CCN(CC(F)F)CC1] |
| | m/z = 673.2 |
| 878 | 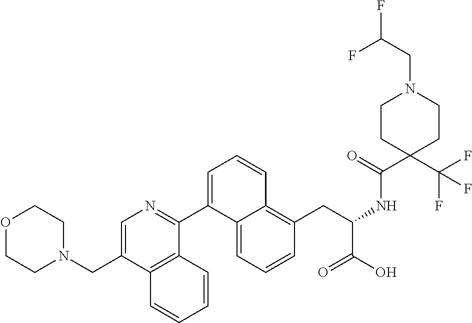 [SMILES: O=C(O)[C@H](Cc1cccc2c(-c3ncc(CN4CCOCC4)c4ccccc34)cccc12)NC(=O)C1(C(F)(F)F)CCN(CC(F)F)CC1] |
| | m/z = 685.2 |
| 879 | 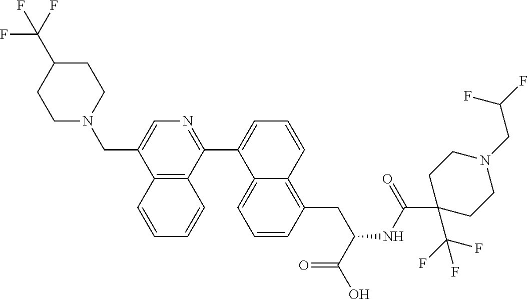 [SMILES: O=C(O)[C@H](Cc1cccc2c(-c3ncc(CN4CCC(C(F)(F)F)CC4)c4ccccc34)cccc12)NC(=O)C1(C(F)(F)F)CCN(CC(F)F)CC1] |
| | m/z = 751.3 |
| 880 | 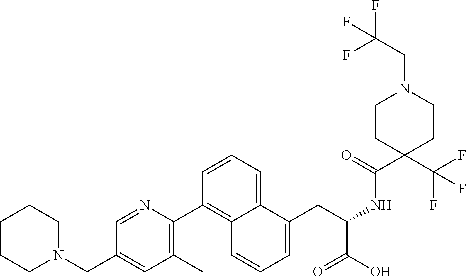 [SMILES: Cc1cc(CN2CCCCC2)cnc1-c1cccc2c(C[C@H](NC(=O)C3(C(F)(F)F)CCN(CC(F)(F)F)CC3)C(=O)O)cccc12] |
| | m/z = 665.3 |
| 881 | 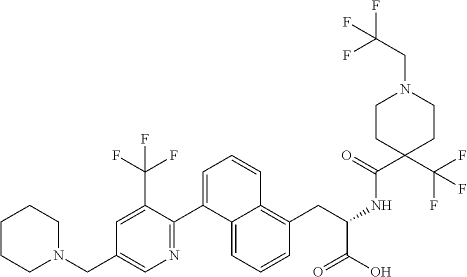 [SMILES: O=C(O)[C@H](Cc1cccc2c(-c3ncc(CN4CCCCC4)cc3C(F)(F)F)cccc12)NC(=O)C1(C(F)(F)F)CCN(CC(F)(F)F)CC1] |
| | m/z = 719.3 |
| 882 | 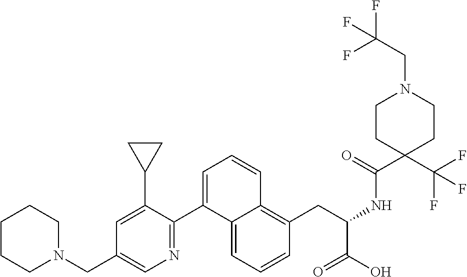 [SMILES: O=C(O)[C@H](Cc1cccc2c(-c3ncc(CN4CCCCC4)cc3C3CC3)cccc12)NC(=O)C1(C(F)(F)F)CCN(CC(F)(F)F)CC1] |
| | m/z = 691.4 |
| 883 | 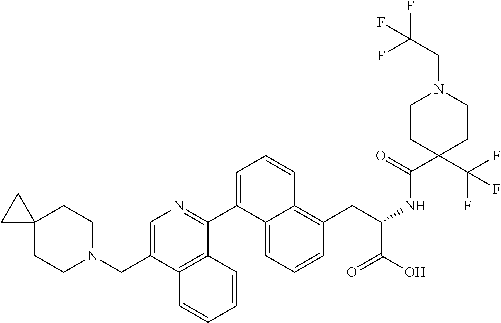 [SMILES: O=C(O)[C@H](Cc1cccc2c(-c3ncc(CN4CCC5(CC4)CC5)c4ccccc34)cccc12)NC(=O)C1(C(F)(F)F)CCN(CC(F)(F)F)CC1] |
| | m/z = 727.2 |
| 884 | 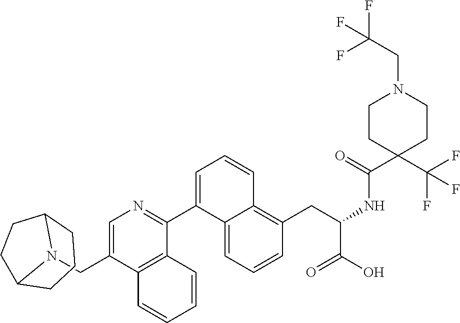 [SMILES: O=C(O)[C@H](Cc1cccc2c(-c3ncc(CN4C5CCCC4CC5)c4ccccc34)cccc12)NC(=O)C1(C(F)(F)F)CCN(CC(F)(F)F)CC1] |
| | m/z = 727.2 |
| 885 | 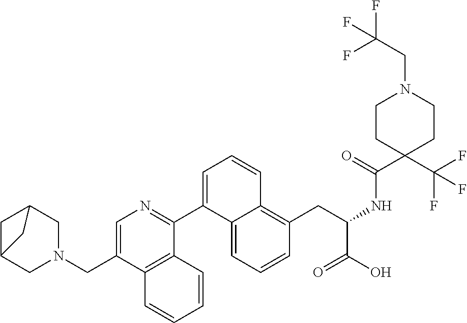 [SMILES: O=C(O)[C@H](Cc1cccc2c(-c3ncc(CN4CC5CC(C5)C4)c4ccccc34)cccc12)NC(=O)C1(C(F)(F)F)CCN(CC(F)(F)F)CC1] |
| | m/z = 713.4 |
| 886 | 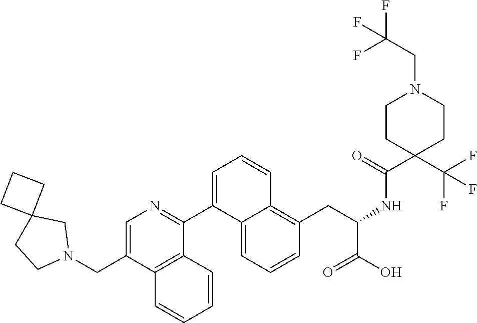 [SMILES: O=C(O)[C@H](Cc1cccc2c(-c3ncc(CN4CCC5(CCC5)C4)c4ccccc34)cccc12)NC(=O)C1(C(F)(F)F)CCN(CC(F)(F)F)CC1] |
| | m/z = 727.4 |
| 887 | 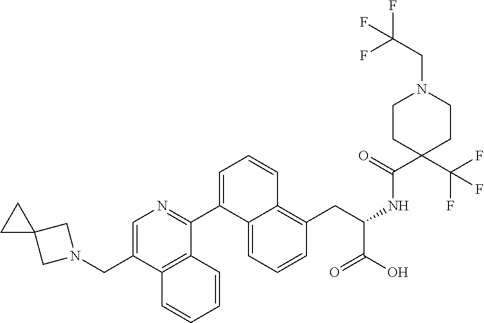 [SMILES: O=C(O)[C@H](Cc1cccc2c(-c3ncc(CN4CC5(CC5)C4)c4ccccc34)cccc12)NC(=O)C1(C(F)(F)F)CCN(CC(F)(F)F)CC1] |
| | m/z = 699.3 |
| 888 | 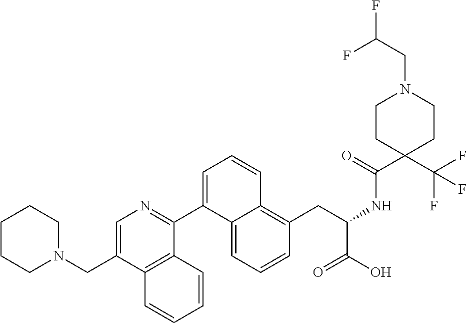 [SMILES: O=C(O)[C@H](Cc1cccc2c(-c3ncc(CN4CCCCC4)c4ccccc34)cccc12)NC(=O)C1(C(F)(F)F)CCN(CC(F)F)CC1] |
| | m/z = 683.3 |
| 889 | 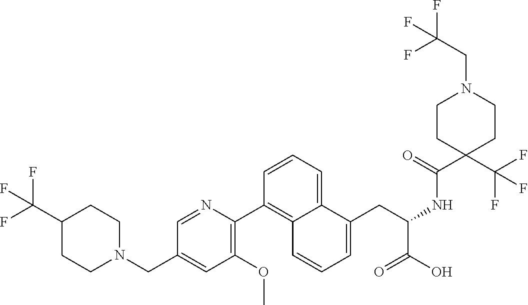 [SMILES: COc1cc(CN2CCC(C(F)(F)F)CC2)cnc1-c1cccc2c(C[C@H](NC(=O)C3(C(F)(F)F)CCN(CC(F)(F)F)CC3)C(=O)O)cccc12] |
| | m/z = 749.3 |
| 890 | 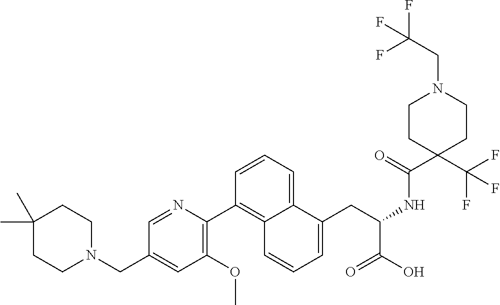 [SMILES: COc1cc(CN2CCC(C)(C)CC2)cnc1-c1cccc2c(C[C@H](NC(=O)C3(C(F)(F)F)CCN(CC(F)(F)F)CC3)C(=O)O)cccc12] |
| | m/z = 709.4 |
| 891 | 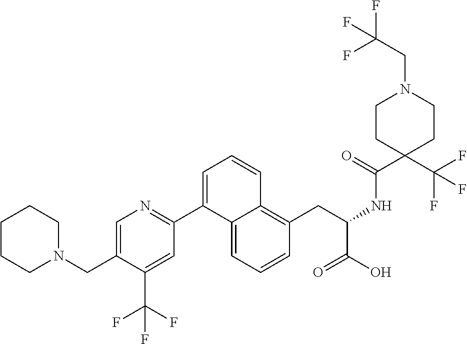 [SMILES: O=C(O)[C@H](Cc1cccc2c(-c3cc(C(F)(F)F)c(CN4CCCCC4)cn3)cccc12)NC(=O)C1(C(F)(F)F)CCN(CC(F)(F)F)CC1] |
| | m/z = 719.2 |
| 892 | 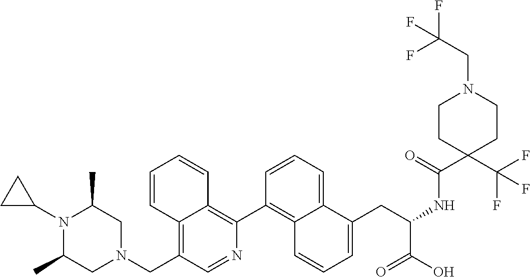 [SMILES: C[C@@H]1CN(Cc2cnc(-c3cccc4c(C[C@H](NC(=O)C5(C(F)(F)F)CCN(CC(F)(F)F)CC5)C(=O)O)cccc34)c3ccccc23)C[C@H](C)N1C1CC1] |
| | m/z = 770.3 |
| 893 | 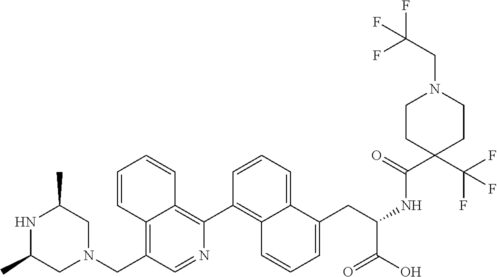 [SMILES: C[C@@H]1CN(Cc2cnc(-c3cccc4c(C[C@H](NC(=O)C5(C(F)(F)F)CCN(CC(F)(F)F)CC5)C(=O)O)cccc34)c3ccccc23)C[C@H](C)N1] |
| | m/z = 730.3 |
| 894 | 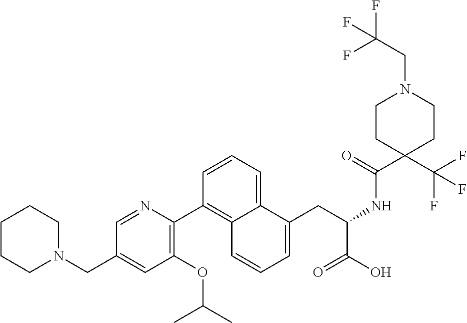 [SMILES: CC(C)Oc1cc(CN2CCCCC2)cnc1-c1cccc2c(C[C@H](NC(=O)C3(C(F)(F)F)CCN(CC(F)(F)F)CC3)C(=O)O)cccc12] |
| | m/z = 709.3 |
| 895 | 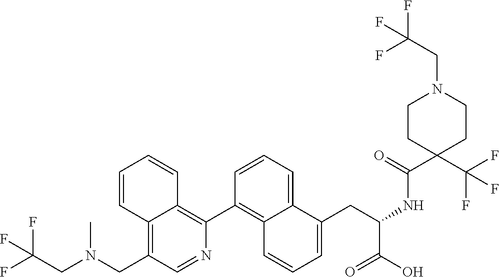 [SMILES: CN(Cc1cnc(-c2cccc3c(C[C@H](NC(=O)C4(C(F)(F)F)CCN(CC(F)(F)F)CC4)C(=O)O)cccc23)c2ccccc12)CC(F)(F)F] |
| | m/z = 729.3 |
| 896 | 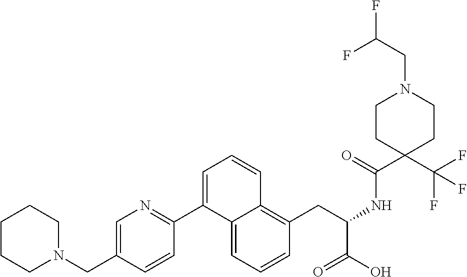 [SMILES: O=C(O)[C@H](Cc1cccc2c(-c3ccc(CN4CCCCC4)cn3)cccc12)NC(=O)C1(C(F)(F)F)CCN(CC(F)F)CC1] |
| | m/z = 633.4 |
| 897 | 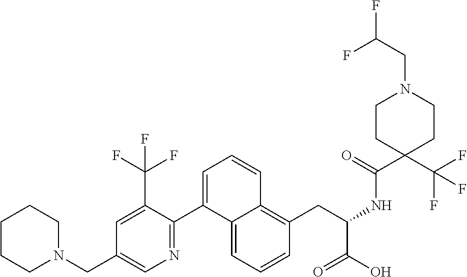 [SMILES: O=C(O)[C@H](Cc1cccc2c(-c3ncc(CN4CCCCC4)cc3C(F)(F)F)cccc12)NC(=O)C1(C(F)(F)F)CCN(CC(F)F)CC1] |
| | m/z = 701.4 |
| 898 | 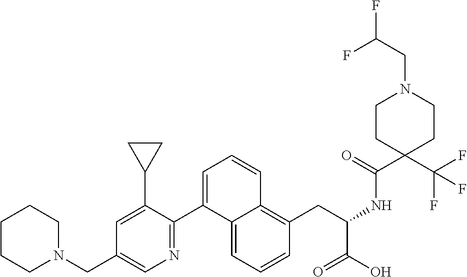 [SMILES: O=C(O)[C@H](Cc1cccc2c(-c3ncc(CN4CCCCC4)cc3C3CC3)cccc12)NC(=O)C1(C(F)(F)F)CCN(CC(F)F)CC1] |
| | m/z = 673.4 |
| 899 | 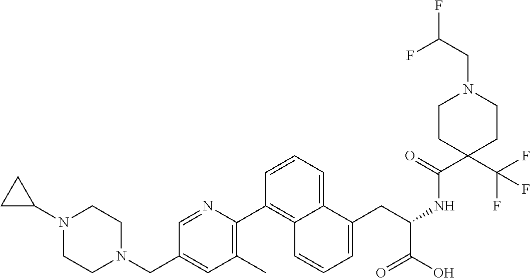 [SMILES: Cc1cc(CN2CCN(C3CC3)CC2)cnc1-c1cccc2c(C[C@H](NC(=O)C3(C(F)(F)F)CCN(CC(F)F)CC3)C(=O)O)cccc12] |
| | m/z = 688.2 |
| 900 | 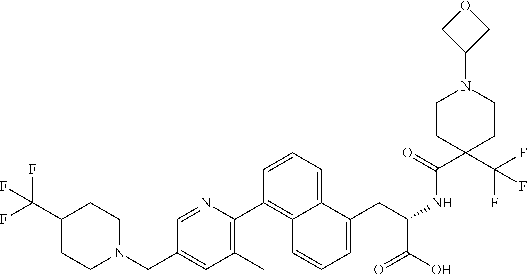 [SMILES: Cc1cc(CN2CCC(C(F)(F)F)CC2)cnc1-c1cccc2c(C[C@H](NC(=O)C3(C(F)(F)F)CCN(C4COC4)CC3)C(=O)O)cccc12] |
| | m/z = 707.4 |
| 901 | 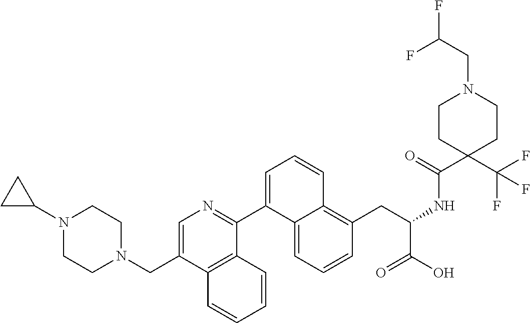 [SMILES: O=C(O)[C@H](Cc1cccc2c(-c3ncc(CN4CCN(C5CC5)CC4)c4ccccc34)cccc12)NC(=O)C1(C(F)(F)F)CCN(CC(F)F)CC1] |
| | m/z = 724.4 |
| 902 | 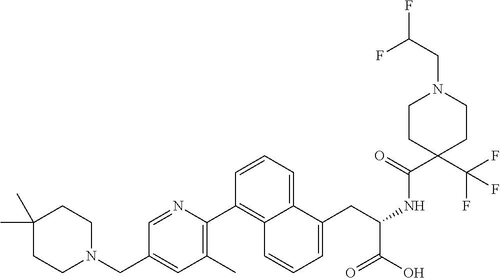 [SMILES: Cc1cc(CN2CCC(C)(C)CC2)cnc1-c1cccc2c(C[C@H](NC(=O)C3(C(F)(F)F)CCN(CC(F)F)CC3)C(=O)O)cccc12] |
| | m/z = 675.4 |
| 903 | 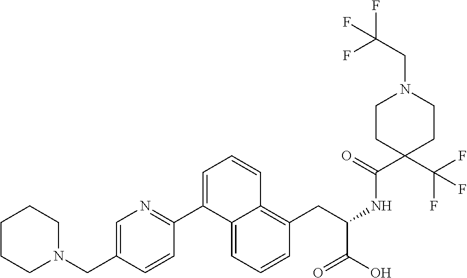 [SMILES: O=C(O)[C@H](Cc1cccc2c(-c3ccc(CN4CCCCC4)cn3)cccc12)NC(=O)C1(C(F)(F)F)CCN(CC(F)(F)F)CC1] |
| | m/z = 651.4 |
| 904 | 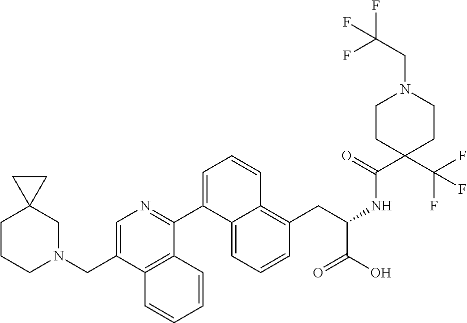 [SMILES: O=C(O)[C@H](Cc1cccc2c(-c3ncc(CN4CCCC5(CC5)C4)c4ccccc34)cccc12)NC(=O)C1(C(F)(F)F)CCN(CC(F)(F)F)CC1] |
| | m/z = 727.5 |
| 905 | 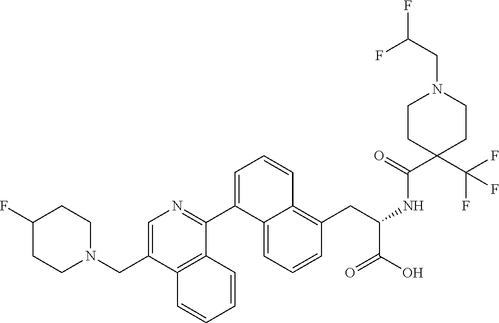 [SMILES: O=C(O)[C@H](Cc1cccc2c(-c3ncc(CN4CCC(F)CC4)c4ccccc34)cccc12)NC(=O)C1(C(F)(F)F)CCN(CC(F)F)CC1] |
| | m/z = 701.3 |
| 906 | 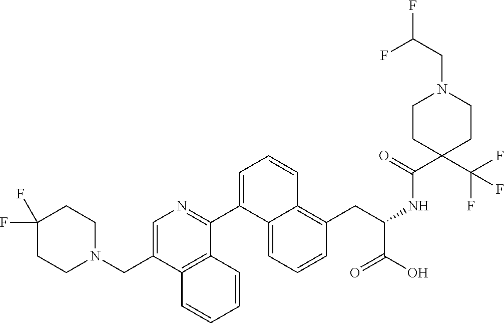 [SMILES: O=C(O)[C@H](Cc1cccc2c(-c3ncc(CN4CCC(F)(F)CC4)c4ccccc34)cccc12)NC(=O)C1(C(F)(F)F)CCN(CC(F)F)CC1] |
| | m/z = 719.2 |
| 907 | 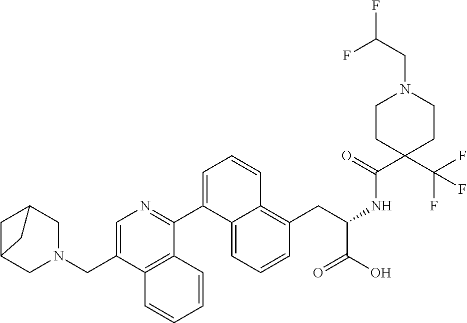 [SMILES: O=C(O)[C@H](Cc1cccc2c(-c3ncc(CN4CC5CC(C5)C4)c4ccccc34)cccc12)NC(=O)C1(C(F)(F)F)CCN(CC(F)F)CC1] |
| | m/z = 695.4 |
| 908 | 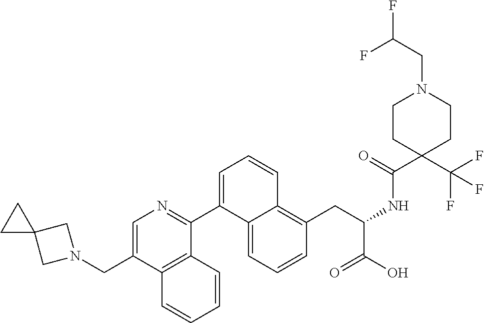 [SMILES: O=C(O)[C@H](Cc1cccc2c(-c3ncc(CN4CC5(CC5)C4)c4ccccc34)cccc12)NC(=O)C1(C(F)(F)F)CCN(CC(F)F)CC1] |
| | m/z = 681.4 |
| 909 | 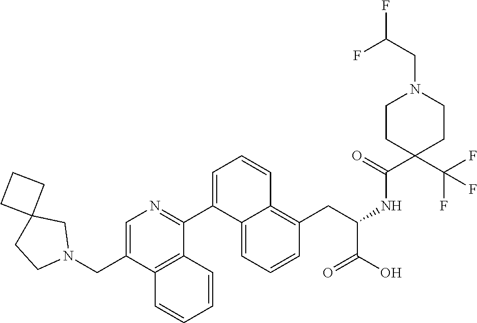 [SMILES: O=C(O)[C@H](Cc1cccc2c(-c3ncc(CN4CCC5(CCC5)C4)c4ccccc34)cccc12)NC(=O)C1(C(F)(F)F)CCN(CC(F)F)CC1] |
| | m/z = 709.4 |
| 910 | 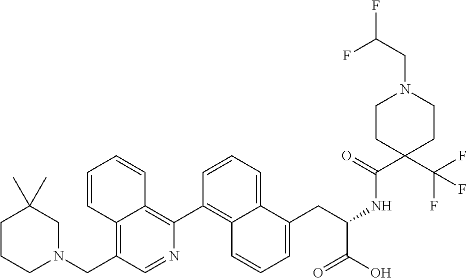 [SMILES: CC1(C)CCCN(Cc2cnc(-c3cccc4c(C[C@H](NC(=O)C5(C(F)(F)F)CCN(CC(F)F)CC5)C(=O)O)cccc34)c3ccccc23)C1] |
| | m/z = 711.4 |
| 911 | 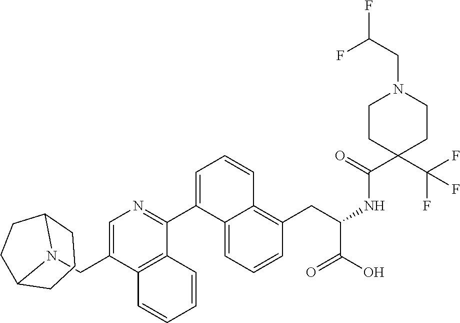 [SMILES: O=C(O)[C@H](Cc1cccc2c(-c3ncc(CN4C5CCCC4CC5)c4ccccc34)cccc12)NC(=O)C1(C(F)(F)F)CCN(CC(F)F)CC1] |
| | m/z = 709.4 |
| 912 | 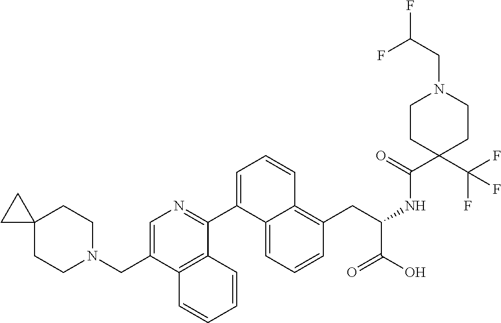 [SMILES: O=C(O)[C@H](Cc1cccc2c(-c3ncc(CN4CCC5(CC4)CC5)c4ccccc34)cccc12)NC(=O)C1(C(F)(F)F)CCN(CC(F)F)CC1] |
| | m/z = 709.5 |
| 913 | 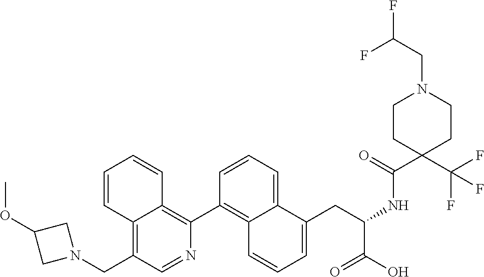 [SMILES: COC1CN(Cc2cnc(-c3cccc4c(C[C@H](NC(=O)C5(C(F)(F)F)CCN(CC(F)F)CC5)C(=O)O)cccc34)c3ccccc23)C1] |
| | m/z = 685.3 |
| 914 | 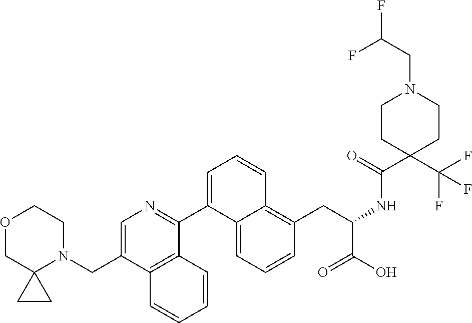 [SMILES: O=C(O)[C@H](Cc1cccc2c(-c3ncc(CN4CCOCC45CC5)c4ccccc34)cccc12)NC(=O)C1(C(F)(F)F)CCN(CC(F)F)CC1] |
| | m/z = 711.5 |
| 915 | 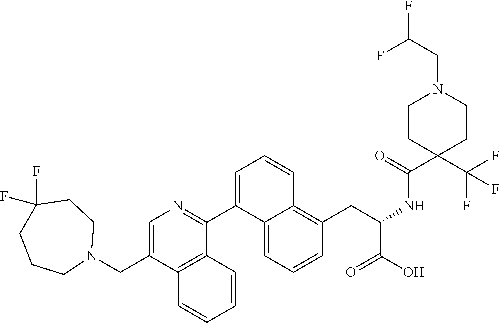 [SMILES: O=C(O)[C@H](Cc1cccc2c(-c3ncc(CN4CCCC(F)(F)CC4)c4ccccc34)cccc12)NC(=O)C1(C(F)(F)F)CCN(CC(F)F)CC1] |
| | m/z = 733.3 |
| 916 | 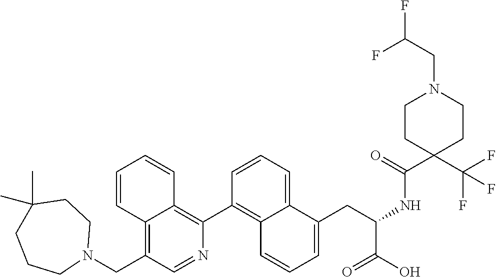 [SMILES: CC1(C)CCCN(Cc2cnc(-c3cccc4c(C[C@H](NC(=O)C5(C(F)(F)F)CCN(CC(F)F)CC5)C(=O)O)cccc34)c3ccccc23)CC1] |
| | m/z = 725.5 |
| 917 | 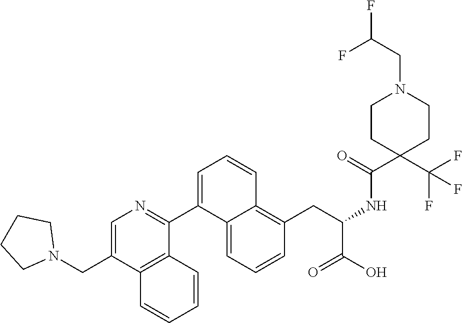 [SMILES: O=C(O)[C@H](Cc1cccc2c(-c3ncc(CN4CCCC4)c4ccccc34)cccc12)NC(=O)C1(C(F)(F)F)CCN(CC(F)F)CC1] |
| | m/z = 669.1 |
| 918 | 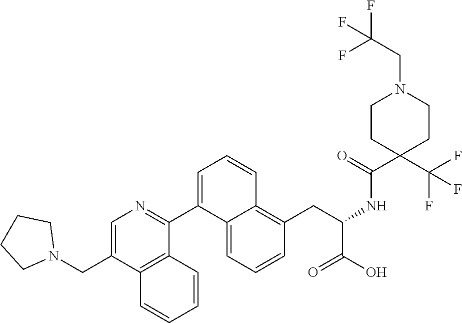 [SMILES: O=C(O)[C@H](Cc1cccc2c(-c3ncc(CN4CCCC4)c4ccccc34)cccc12)NC(=O)C1(C(F)(F)F)CCN(CC(F)(F)F)CC1] |
| | m/z = 687.3 |
| 919 | 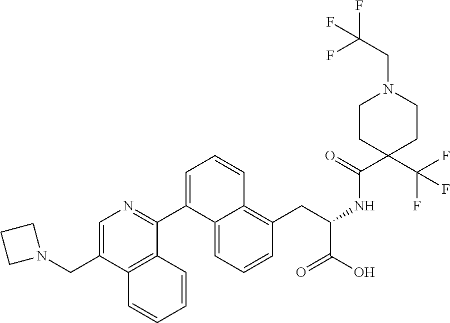 [SMILES: O=C(O)[C@H](Cc1cccc2c(-c3ncc(CN4CCC4)c4ccccc34)cccc12)NC(=O)C1(C(F)(F)F)CCN(CC(F)(F)F)CC1] |
| | m/z = 673.4 |
| 920 | 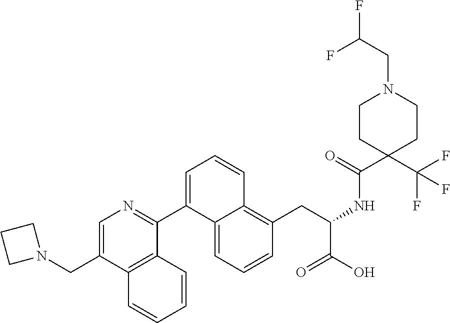 [SMILES: O=C(O)[C@H](Cc1cccc2c(-c3ncc(CN4CCC4)c4ccccc34)cccc12)NC(=O)C1(C(F)(F)F)CCN(CC(F)F)CC1] |
| | m/z = 655.1 |
| 921 | 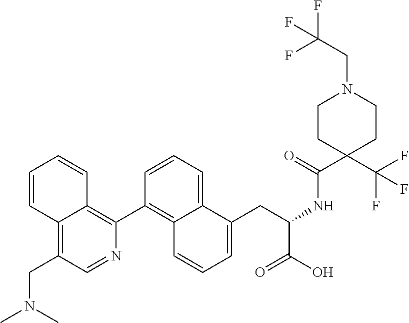 [SMILES: CN(C)Cc1cnc(-c2cccc3c(C[C@H](NC(=O)C4(C(F)(F)F)CCN(CC(F)(F)F)CC4)C(=O)O)cccc23)c2ccccc12] |
| | m/z = 661.3 |
| 922 | 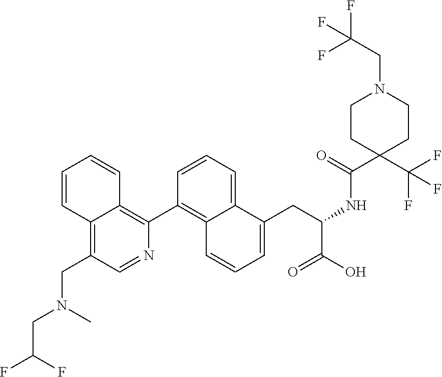 [SMILES: CN(Cc1cnc(-c2cccc3c(C[C@H](NC(=O)C4(C(F)(F)F)CCN(CC(F)(F)F)CC4)C(=O)O)cccc23)c2ccccc12)CC(F)F] |
| | m/z = 711.4 |
| 923 | 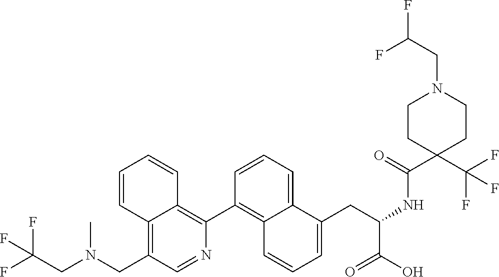 [SMILES: CN(Cc1cnc(-c2cccc3c(C[C@H](NC(=O)C4(C(F)(F)F)CCN(CC(F)F)CC4)C(=O)O)cccc23)c2ccccc12)CC(F)(F)F] |
| | m/z = 711.1 |
| 924 | 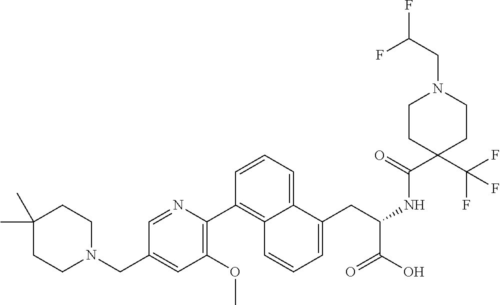 [SMILES: COc1cc(CN2CCC(C)(C)CC2)cnc1-c1cccc2c(C[C@H](NC(=O)C3(C(F)(F)F)CCN(CC(F)F)CC3)C(=O)O)cccc12] |
| | m/z = 691.7 |
| 925 | 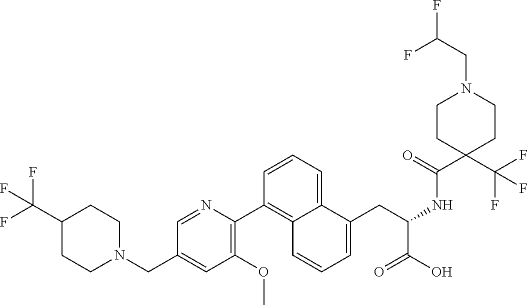 [SMILES: COc1cc(CN2CCC(C(F)(F)F)CC2)cnc1-c1cccc2c(C[C@H](NC(=O)C3(C(F)(F)F)CCN(CC(F)F)CC3)C(=O)O)cccc12] |
| | m/z = 731.2 |
| 926* | 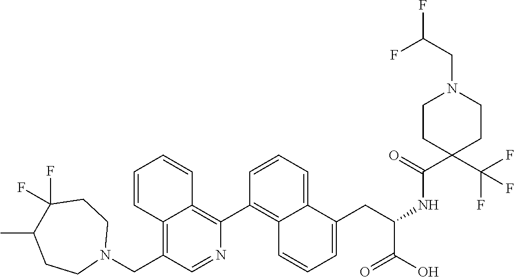 [SMILES: CC1CCN(Cc2cnc(-c3cccc4c(C[C@H](NC(=O)C5(C(F)(F)F)CCN(CC(F)F)CC5)C(=O)O)cccc34)c3ccccc23)CCC1(F)F] |
| | m/z = 747.1 |
| 927 | 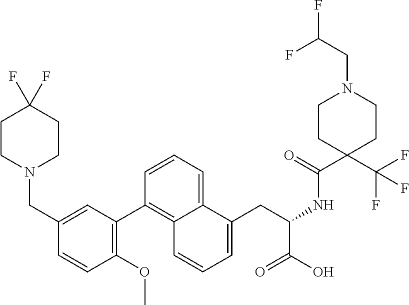 [SMILES: COc1ccc(CN2CCC(F)(F)CC2)cc1-c1cccc2c(C[C@H](NC(=O)C3(C(F)(F)F)CCN(CC(F)F)CC3)C(=O)O)cccc12] |
| | m/z = 698.3 |
| 928 | 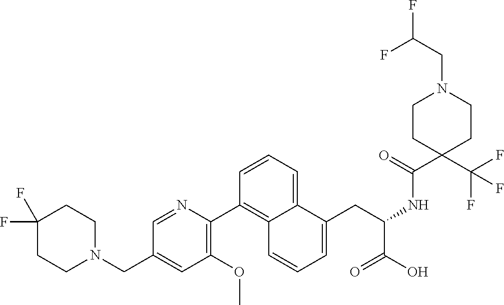 [SMILES: COc1cc(CN2CCC(F)(F)CC2)cnc1-c1cccc2c(C[C@H](NC(=O)C3(C(F)(F)F)CCN(CC(F)F)CC3)C(=O)O)cccc12] |
| | m/z = 699.3 |
| 929 | 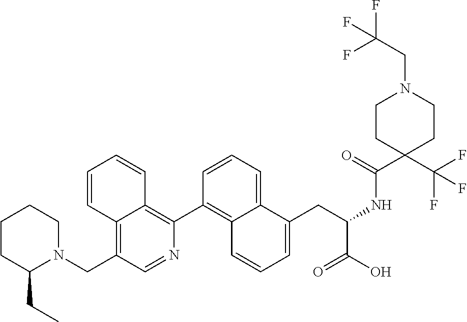 [SMILES: CC[C@H]1CCCCN1Cc1cnc(-c2cccc3c(C[C@H](NC(=O)C4(C(F)(F)F)CCN(CC(F)(F)F)CC4)C(=O)O)cccc23)c2ccccc12] |
| | m/z = 729.2 |
| 930 | 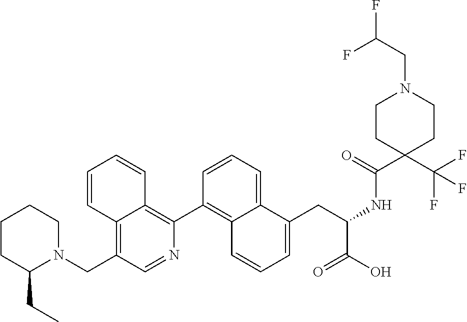 [SMILES: CC[C@H]1CCCCN1Cc1cnc(-c2cccc3c(C[C@H](NC(=O)C4(C(F)(F)F)CCN(CC(F)F)CC4)C(=O)O)cccc23)c2ccccc12] |
| | m/z = 711.3 |
| 931 | 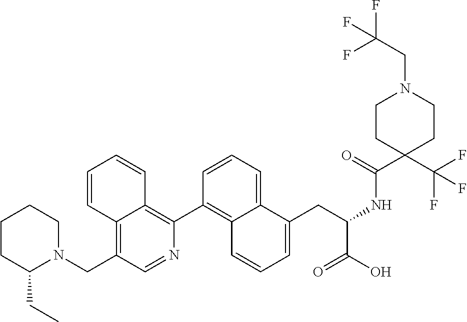 [SMILES: CC[C@@H]1CCCCN1Cc1cnc(-c2cccc3c(C[C@H](NC(=O)C4(C(F)(F)F)CCN(CC(F)(F)F)CC4)C(=O)O)cccc23)c2ccccc12] |
| | m/z = 729.4 |
| 932 | 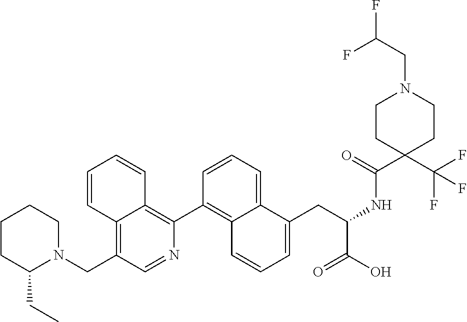 [SMILES: CC[C@@H]1CCCCN1Cc1cnc(-c2cccc3c(C[C@H](NC(=O)C4(C(F)(F)F)CCN(CC(F)F)CC4)C(=O)O)cccc23)c2ccccc12] |
| | m/z = 711.3 |
| 933 | 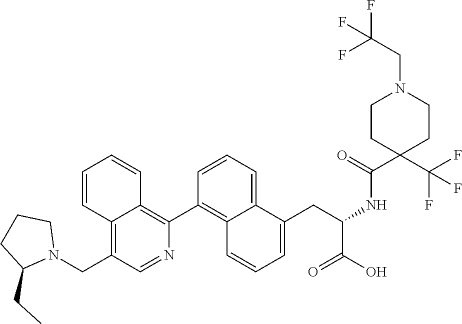 [SMILES: CC[C@H]1CCCN1Cc1cnc(-c2cccc3c(C[C@H](NC(=O)C4(C(F)(F)F)CCN(CC(F)(F)F)CC4)C(=O)O)cccc23)c2ccccc12] |
| | m/z = 715.3 |
| 934 | 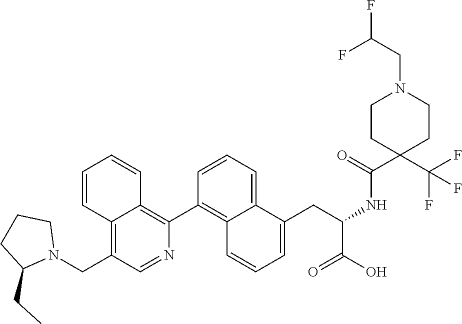 [SMILES: CC[C@H]1CCCN1Cc1cnc(-c2cccc3c(C[C@H](NC(=O)C4(C(F)(F)F)CCN(CC(F)F)CC4)C(=O)O)cccc23)c2ccccc12] |
| | m/z = 697.3 |
| 935 | 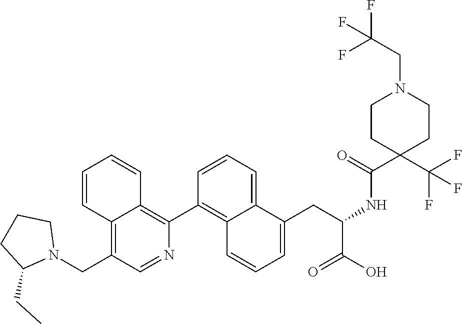 [SMILES: CC[C@@H]1CCCN1Cc1cnc(-c2cccc3c(C[C@H](NC(=O)C4(C(F)(F)F)CCN(CC(F)(F)F)CC4)C(=O)O)cccc23)c2ccccc12] |
| | m/z = 715.3 |
| 936 | 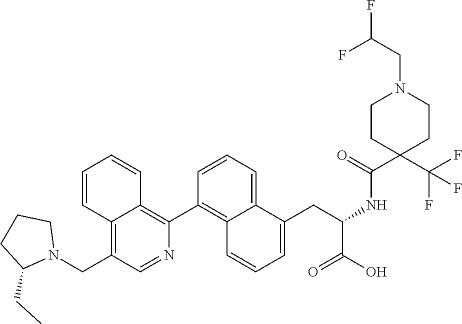 [SMILES: CC[C@@H]1CCCN1Cc1cnc(-c2cccc3c(C[C@H](NC(=O)C4(C(F)(F)F)CCN(CC(F)F)CC4)C(=O)O)cccc23)c2ccccc12] |
| | m/z = 697.3 |
| 937 | 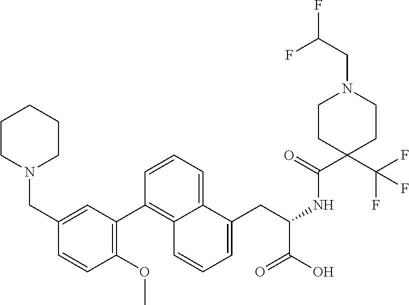 [SMILES: COc1ccc(CN2CCCCC2)cc1-c1cccc2c(C[C@H](NC(=O)C3(C(F)(F)F)CCN(CC(F)F)CC3)C(=O)O)cccc12] |
| | m/z = 662.3 |
| 938 | 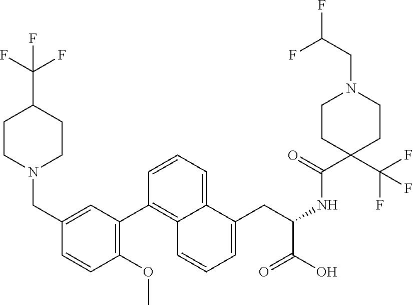 [SMILES: COc1ccc(CN2CCC(C(F)(F)F)CC2)cc1-c1cccc2c(C[C@H](NC(=O)C3(C(F)(F)F)CCN(CC(F)F)CC3)C(=O)O)cccc12] |
| | m/z = 730.3 |
| 939 | 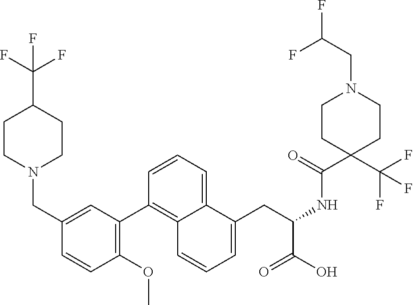 [SMILES: COc1ccc(CN2CCC(C(F)(F)F)CC2)cc1-c1cccc2c(C[C@H](NC(=O)C3(C(F)(F)F)CCN(CC(F)F)CC3)C(=O)O)cccc12] |
| | m/z = 730.1 |
| 940 | 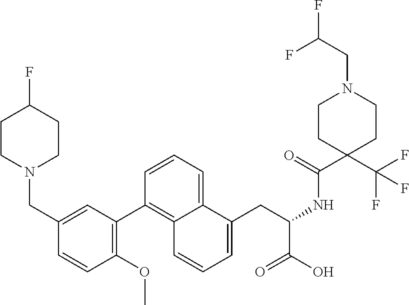 [SMILES: COc1ccc(CN2CCC(F)CC2)cc1-c1cccc2c(C[C@H](NC(=O)C3(C(F)(F)F)CCN(CC(F)F)CC3)C(=O)O)cccc12] |
| | m/z = 680.3 |
| 941 | 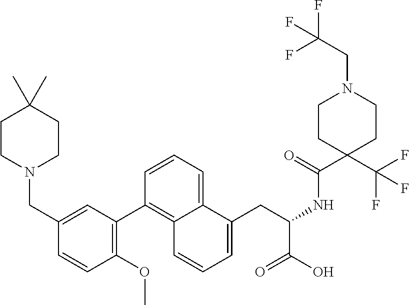 [SMILES: COc1ccc(CN2CCC(C)(C)CC2)cc1-c1cccc2c(C[C@H](NC(=O)C3(C(F)(F)F)CCN(CC(F)(F)F)CC3)C(=O)O)cccc12] |
| | m/z = 708.4 |
| 942 | 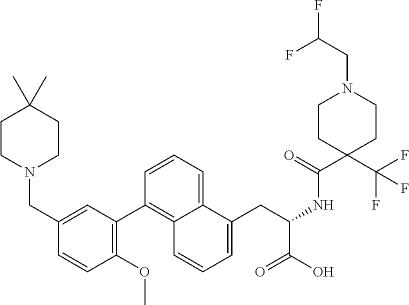 [SMILES: COc1ccc(CN2CCC(C)(C)CC2)cc1-c1cccc2c(C[C@H](NC(=O)C3(C(F)(F)F)CCN(CC(F)F)CC3)C(=O)O)cccc12] |
| | m/z = 690.3 |
| 943 | 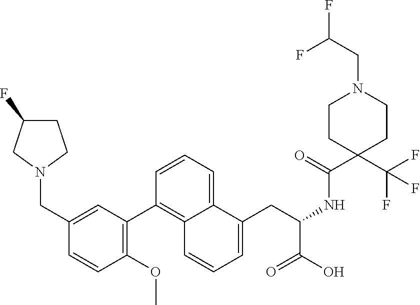 [SMILES: COc1ccc(CN2CC[C@H](F)C2)cc1-c1cccc2c(C[C@H](NC(=O)C3(C(F)(F)F)CCN(CC(F)F)CC3)C(=O)O)cccc12] |
| | m/z = 666.3 |
| 944 | 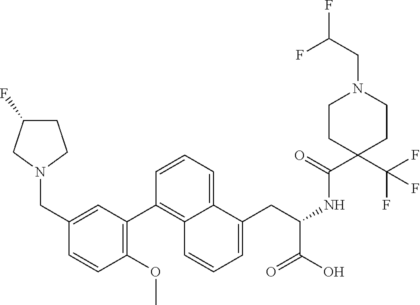 [SMILES: COc1ccc(CN2CC[C@@H](F)C2)cc1-c1cccc2c(C[C@H](NC(=O)C3(C(F)(F)F)CCN(CC(F)F)CC3)C(=O)O)cccc12] |
| | m/z = 666.3 |
| 945 | 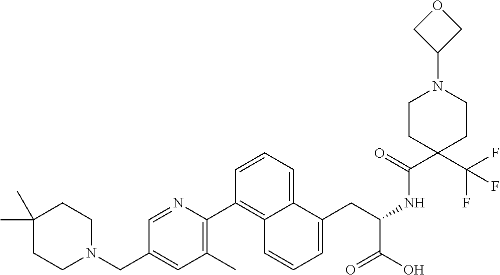 [SMILES: Cc1cc(CN2CCC(C)(C)CC2)cnc1-c1cccc2c(C[C@H](NC(=O)C3(C(F)(F)F)CCN(C4COC4)CC3)C(=O)O)cccc12] |
| | m/z = 667.4 |
| 946 | 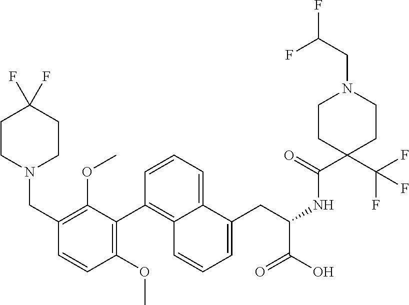 [SMILES: COc1ccc(CN2CCC(F)(F)CC2)c(OC)c1-c1cccc2c(C[C@H](NC(=O)C3(C(F)(F)F)CCN(CC(F)F)CC3)C(=O)O)cccc12] |
| | m/z = 728.4 |
| 947 | 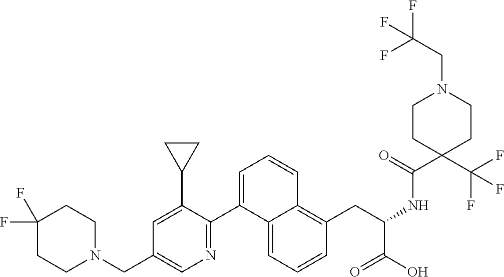 [SMILES: O=C(O)[C@H](Cc1cccc2c(-c3ncc(CN4CCC(F)(F)CC4)cc3C3CC3)cccc12)NC(=O)C1(C(F)(F)F)CCN(CC(F)(F)F)CC1] |
| | m/z = 727.4 |
| 948 | 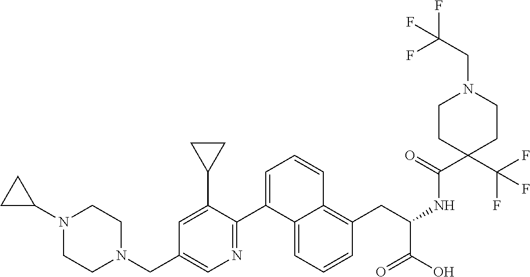 [SMILES: O=C(O)[C@H](Cc1cccc2c(-c3ncc(CN4CCN(C5CC5)CC4)cc3C3CC3)cccc12)NC(=O)C1(C(F)(F)F)CCN(CC(F)(F)F)CC1] |
| | m/z = 732.3 |
| 949 | 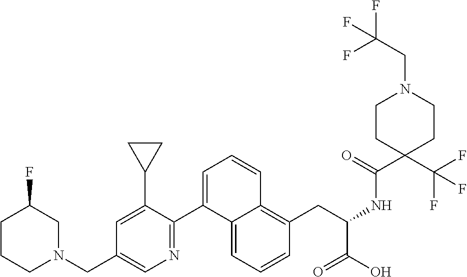 [SMILES: O=C(O)[C@H](Cc1cccc2c(-c3ncc(CN4CCC[C@@H](F)C4)cc3C3CC3)cccc12)NC(=O)C1(C(F)(F)F)CCN(CC(F)(F)F)CC1] |
| | m/z = 709.4 |
| 950 | 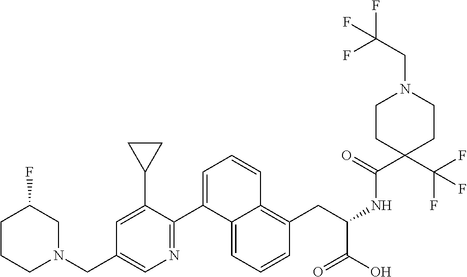 [SMILES: O=C(O)[C@H](Cc1cccc2c(-c3ncc(CN4CCC[C@H](F)C4)cc3C3CC3)cccc12)NC(=O)C1(C(F)(F)F)CCN(CC(F)(F)F)CC1] |
| | m/z = 709.3 |
| 951 | 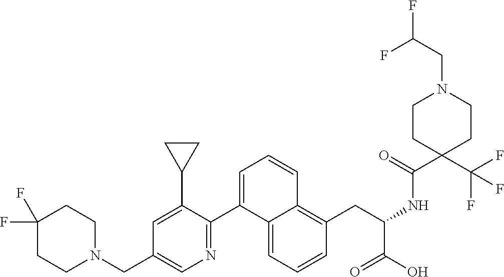 [SMILES: O=C(O)[C@H](Cc1cccc2c(-c3ncc(CN4CCC(F)(F)CC4)cc3C3CC3)cccc12)NC(=O)C1(C(F)(F)F)CCN(CC(F)F)CC1] |
| | m/z = 709.2 |
| 952 | 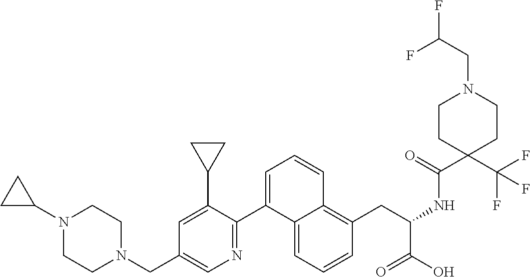 [SMILES: O=C(O)[C@H](Cc1cccc2c(-c3ncc(CN4CCN(C5CC5)CC4)cc3C3CC3)cccc12)NC(=O)C1(C(F)(F)F)CCN(CC(F)F)CC1] |
| | m/z = 714.5 |
| 953 | 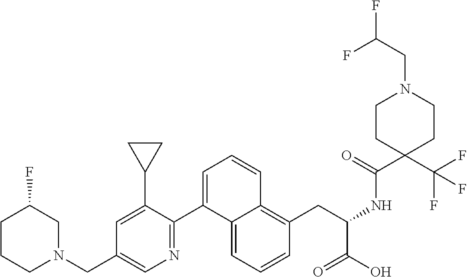 [SMILES: O=C(O)[C@H](Cc1cccc2c(-c3ncc(CN4CCC[C@H](F)C4)cc3C3CC3)cccc12)NC(=O)C1(C(F)(F)F)CCN(CC(F)F)CC1] |
| | m/z = 691.2 |
| 954 | 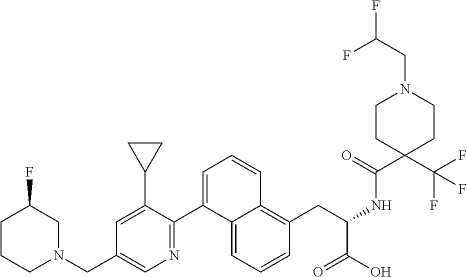 [SMILES: O=C(O)[C@H](Cc1cccc2c(-c3ncc(CN4CCC[C@@H](F)C4)cc3C3CC3)cccc12)NC(=O)C1(C(F)(F)F)CCN(CC(F)F)CC1] |
| | m/z = 691.2 |

*Denotes a racemic compound;
**Denotes a single isomer with unknown absolute stereochemistry

### Example 42: General Scheme 2C

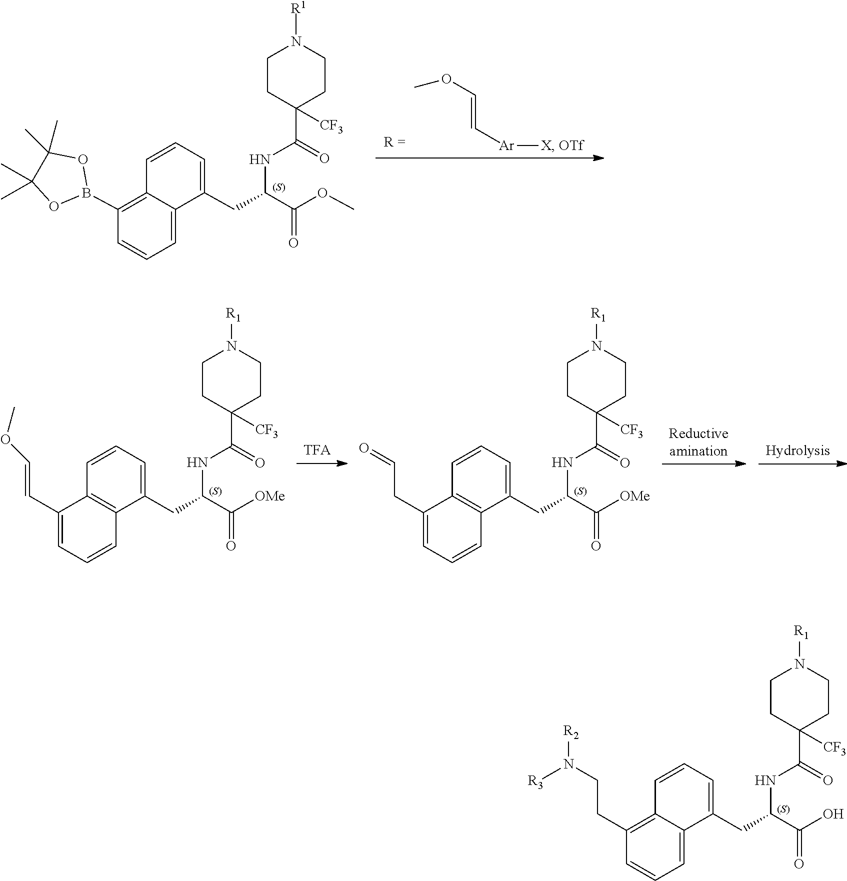

The proceeding compounds were synthesized according to the procedures described in General Scheme 2C using Common Intermediate 2 or Int2 as an intermediate.

### Synthesis of Compound 955

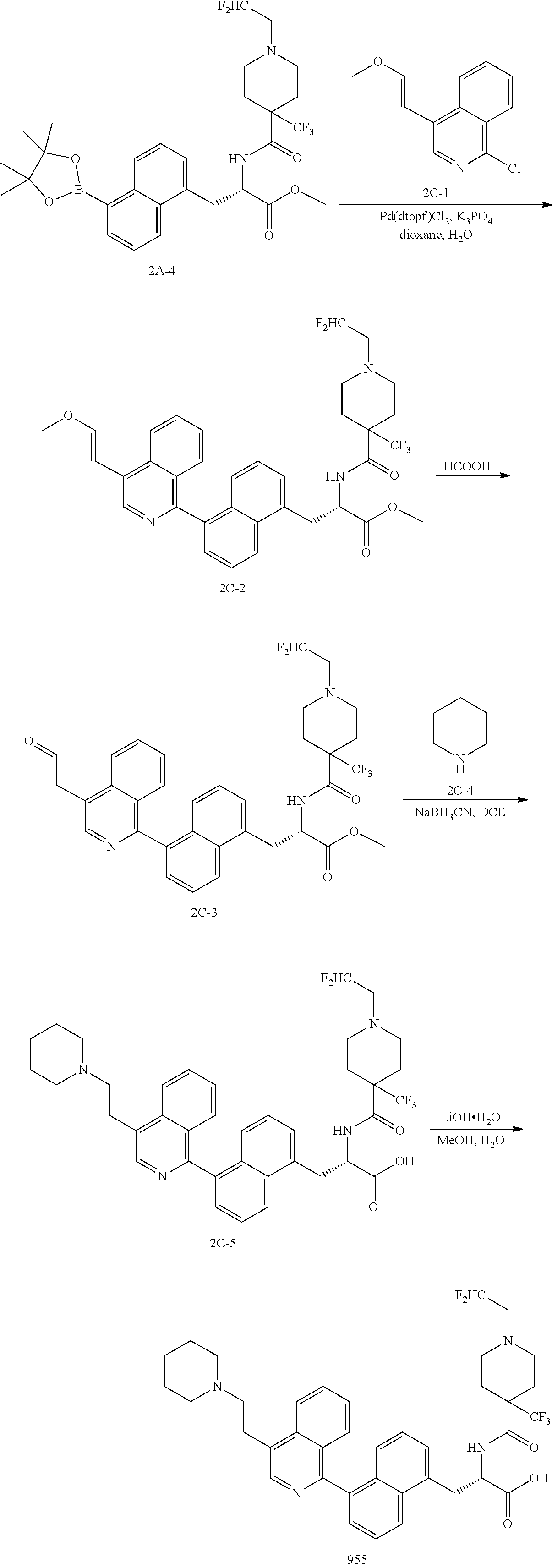

Step 1: A mixture of compound 2A-4 (150 mg, 251 μmol, 1.00 eq), compound 2C-1 (65.0 mg, 296 μmol, 1.18 eq), Pd(dtbpf)Cl₂ (17.0 mg, 26.1 μmol, 0.100 eq) and K₃PO₄ (160 mg, 754 μmol, 3.01 eq) in dioxane (5.00 mL) and H₂O (1.50 mL) was degassed and purged with N₂ for 3 times, and then the mixture was stirred at 25° C. for 1.5 hrs under N₂ atmosphere. The reaction mixture was diluted with H₂O (20.0 mL) and extracted with ethyl acetate 45.0 mL (15.0 mL*3). The combined organic layers were washed by brine 15.0 mL (5.00 mL*3), dried over Na₂SO₄, filtered, and concentrated under reduced pressure to give a residue. The residue was purified by Prep-TLC (SiO₂, Petroleum ether:Ethyl acetate=1:1, R_{f}=0.39). Compound 2C-2 (148 mg, 226 μmol, 90.0% yield) was obtained as colorless gum. LC-MS: (M+H)⁺: 656.2.

Step 2: A mixture of compound 2C-2 (20.0 mg, 30.5 μmol, 1.00 eq) and HCOOH (0.500 mL) was stirred at 50° C. for 2.5 hrs. The reaction mixture was concentrated under reduced pressure to remove HCOOH. The residue was diluted with H₂O (10.0 mL), and the pH of aqueous phase was adjusted to 7 with aq. NaHCO₃. The aqueous layer was extracted with ethyl acetate 30.0 mL (10.0 mL*3). The combined organic layers were washed with brine (5.00 mL), dried over Na₂SO₄, filtered, and concentrated under reduced pressure to give a residue. Compound 2C-3 (80.0 mg, crude) was obtained as yellow oil. LC-MS: (M+H)⁺: 642.2.

Step 3: To a solution of compound 2C-3 (80.0 mg, 125 μmol, 1.00 eq) in DCE (3.00 mL) was added 2C-4 (40.0 mg, 470 μmol, 46.4 μL, 3.77 eq). The mixture was stirred at 25° C. for 12 hrs. Then NaBH₃CN (12.0 mg, 191 μmol, 1.53 eq) was added, and the mixture was stirred at 25° C. for 1 hr. The reaction mixture was diluted with H₂O (20.0 mL), extracted with ethyl acetate 45.0 mL (15.0 mL*3). The combined organic layers were washed with brine (5.00 mL), dried over Na 2SO₄, filtered, and concentrated under reduced pressure to give a residue. The residue was purified by Prep-TLC (SiO₂, Dichloromethane:Methanol=10:1, R_{f}=0.53). Compound 2C-5 (50.0 mg, 70.6 μmol, 56.4% yield) was obtained as yellow oil. LC-MS: (M+H)⁺: 711.3.

Step 4: To a solution of compound 2C-5 (40.0 mg, 56.3 μmol, 1.00 eq) in MeOH (2.00 mL) and H₂O (0.500 mL) was added LiOH·H₂O (4.00 mg, 95.3 μmol, 1.69 eq). The mixture was stirred at 25° C. for 1 hr. The reaction mixture was concentrated under reduced pressure to give a residue. The residue was purified by Prep-HPLC (column: Waters xbridge 150*25 mm 10 um; mobile phase: [water (NH₄HCO₃)-ACN]; gradient: 22%-52% B over 10 min). Compound 955 (32.36 mg, 46.2 μmol, 82.0% yield, 99.4% purity) was obtained as white solid. LC-MS: (M+H)⁺: 697.4. ¹H NMR: (400 MHz, DMSO-d₆) δ 8.55 (s, 1H), 8.42-8.28 (m, 2H), 8.21 (d, J=8.0 Hz, 1H), 7.85-7.70 (m, 2H), 7.54 (d, J=6.8 Hz, 1H), 7.50-7.35 (m, 3H), 7.25-7.16 (m, 1H), 7.03 (br d, J=8.4 Hz, 1H), 6.20-5.78 (m, 1H), 4.87-4.65 (m, 1H), 3.89-3.79 (m, 1H), 3.55-3.45 (m, 3H), 2.83 (br s, 2H), 2.75-2.59 (m, 6H), 2.41-2.20 (m, 3H), 2.01-1.72 (m, 2H), 1.69-1.53 (m, 7H), 1.51-1.43 (m, 2H).

The compounds set forth in Table 22, were prepared using the synthetic procedures of Example 42, or analogous procedures as provided herein.

TABLE 22
Structure and Spectroscopic data for Compounds 956 to 985

| No. | Structure |
|---|---|
| 956 | 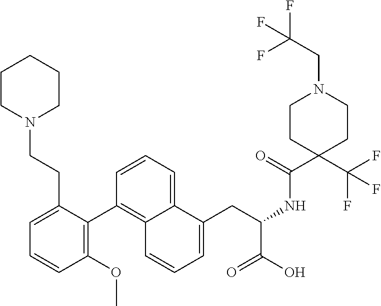 [SMILES: COc1cccc(CCN2CCCCC2)c1-c1cccc2c(C[C@H](NC(=O)C3(C(F)(F)F)CCN(CC(F)(F)F)CC3)C(=O)O)cccc12] |
| | m/z = 694.3 |
| 957 | 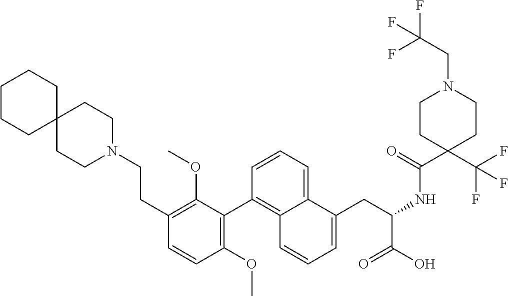 [SMILES: COc1ccc(CCN2CCC3(CCCCC3)CC2)c(OC)c1-c1cccc2c(C[C@H](NC(=O)C3(C(F)(F)F)CCN(CC(F)(F)F)CC3)C(=O)O)cccc12] |
| | m/z = 792.3 |
| 958 | 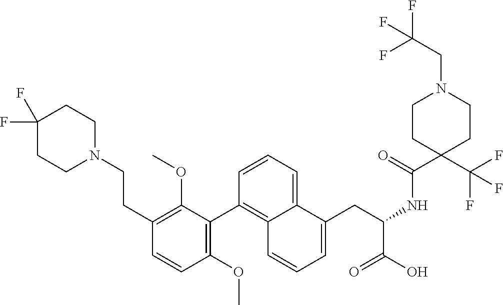 [SMILES: COc1ccc(CCN2CCC(F)(F)CC2)c(OC)c1-c1cccc2c(C[C@H](NC(=O)C3(C(F)(F)F)CCN(CC(F)(F)F)CC3)C(=O)O)cccc12] |
| | m/z = 760.2 |
| 959* | 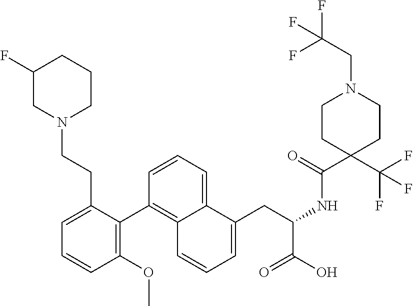 [SMILES: COc1cccc(CCN2CCCC(F)C2)c1-c1cccc2c(C[C@H](NC(=O)C3(C(F)(F)F)CCN(CC(F)(F)F)CC3)C(=O)O)cccc12] |
| | m/z = 712.1 |
| 960 | 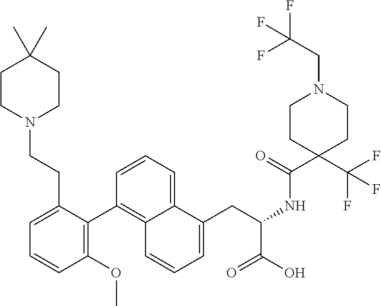 [SMILES: COc1cccc(CCN2CCC(C)(C)CC2)c1-c1cccc2c(C[C@H](NC(=O)C3(C(F)(F)F)CCN(CC(F)(F)F)CC3)C(=O)O)cccc12] |
| | m/z = 722.1 |
| 961 | 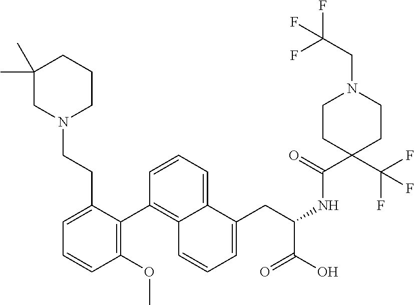 [SMILES: COc1cccc(CCN2CCCC(C)(C)C2)c1-c1cccc2c(C[C@H](NC(=O)C3(C(F)(F)F)CCN(CC(F)(F)F)CC3)C(=O)O)cccc12] |
| | m/z = 722.3 |
| 962 | 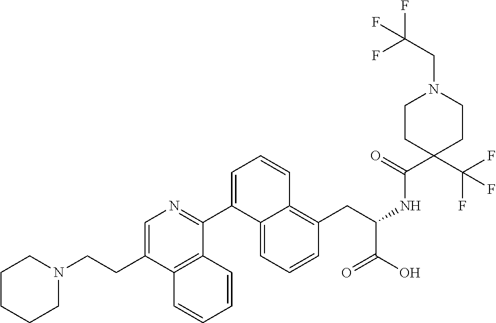 [SMILES: O=C(O)[C@H](Cc1cccc2c(-c3ncc(CCN4CCCCC4)c4ccccc34)cccc12)NC(=O)C1(C(F)(F)F)CCN(CC(F)(F)F)CC1] |
| | m/z = 715.5 |
| 963 | 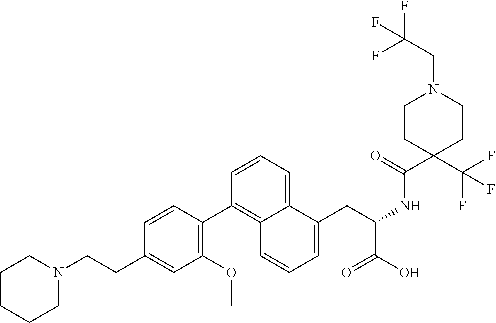 [SMILES: COc1cc(CCN2CCCCC2)ccc1-c1cccc2c(C[C@H](NC(=O)C3(C(F)(F)F)CCN(CC(F)(F)F)CC3)C(=O)O)cccc12] |
| | m/z = 694.4 |
| 964 | 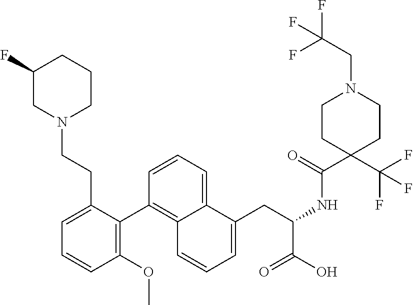 [SMILES: COc1cccc(CCN2CCC[C@H](F)C2)c1-c1cccc2c(C[C@H](NC(=O)C3(C(F)(F)F)CCN(CC(F)(F)F)CC3)C(=O)O)cccc12] |
| | m/z = 712.2 |
| 965 | 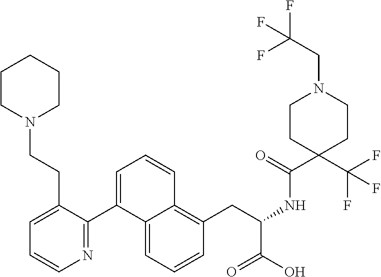 [SMILES: O=C(O)[C@H](Cc1cccc2c(-c3ncccc3CCN3CCCCC3)cccc12)NC(=O)C1(C(F)(F)F)CCN(CC(F)(F)F)CC1] |
| | m/z = 665.1 |
| 966 | 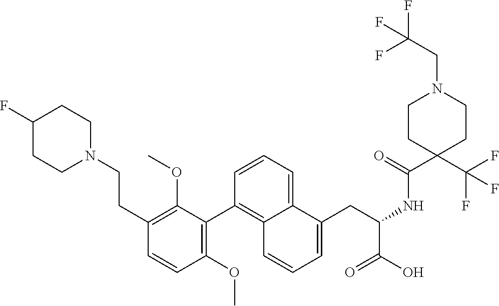 [SMILES: COc1ccc(CCN2CCC(F)CC2)c(OC)c1-c1cccc2c(C[C@H](NC(=O)C3(C(F)(F)F)CCN(CC(F)(F)F)CC3)C(=O)O)cccc12] |
| | m/z = 742 |
| 967 | 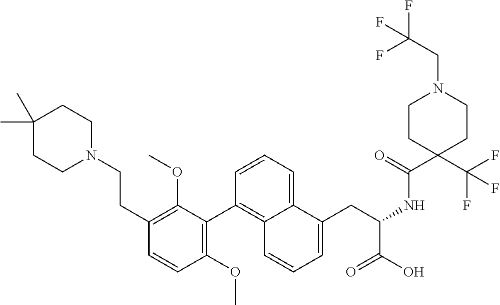 [SMILES: COc1ccc(CCN2CCC(C)(C)CC2)c(OC)c1-c1cccc2c(C[C@H](NC(=O)C3(C(F)(F)F)CCN(CC(F)(F)F)CC3)C(=O)O)cccc12] |
| | m/z = 752.4 |
| 968 | 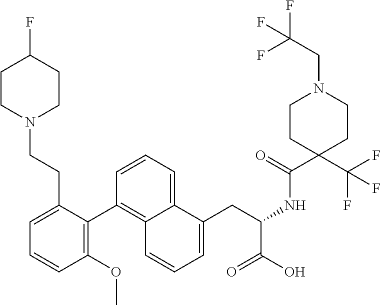 [SMILES: COc1cccc(CCN2CCC(F)CC2)c1-c1cccc2c(C[C@H](NC(=O)C3(C(F)(F)F)CCN(CC(F)(F)F)CC3)C(=O)O)cccc12] |
| | m/z = 712.3 |
| 969 | 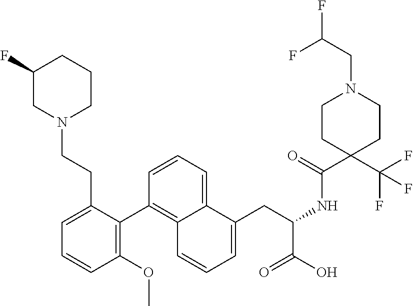 [SMILES: COc1cccc(CCN2CCC[C@H](F)C2)c1-c1cccc2c(C[C@H](NC(=O)C3(C(F)(F)F)CCN(CC(F)F)CC3)C(=O)O)cccc12] |
| | m/z = 694.4 |
| 970 | 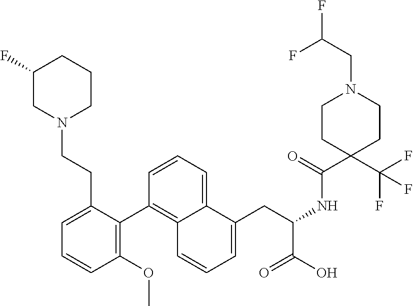 [SMILES: COc1cccc(CCN2CCC[C@@H](F)C2)c1-c1cccc2c(C[C@H](NC(=O)C3(C(F)(F)F)CCN(CC(F)F)CC3)C(=O)O)cccc12] |
| | m/z = 694.4 |
| 971* | 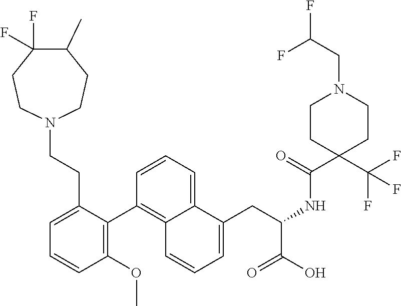 [SMILES: COc1cccc(CCN2CCC(C)C(F)(F)CC2)c1-c1cccc2c(C[C@H](NC(=O)C3(C(F)(F)F)CCN(CC(F)F)CC3)C(=O)O)cccc12] |
| | m/z = 740.4 |
| 972* | 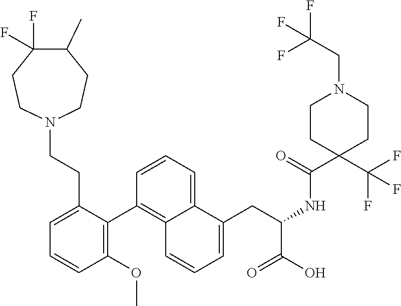 [SMILES: COc1cccc(CCN2CCC(C)C(F)(F)CC2)c1-c1cccc2c(C[C@H](NC(=O)C3(C(F)(F)F)CCN(CC(F)(F)F)CC3)C(=O)O)cccc12] |
| | m/z = 758.1 |
| 973 | 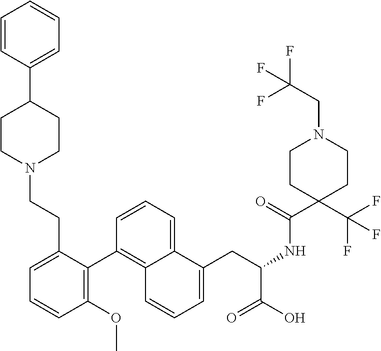 [SMILES: COc1cccc(CCN2CCC(c3ccccc3)CC2)c1-c1cccc2c(C[C@H](NC(=O)C3(C(F)(F)F)CCN(CC(F)(F)F)CC3)C(=O)O)cccc12] |
| | m/z = 770.3 |
| 974 | 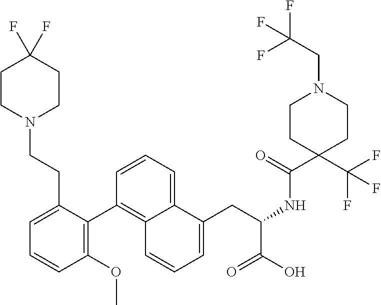 [SMILES: COc1cccc(CCN2CCC(F)(F)CC2)c1-c1cccc2c(C[C@H](NC(=O)C3(C(F)(F)F)CCN(CC(F)(F)F)CC3)C(=O)O)cccc12] |
| | m/z = 730.3 |
| 975 | 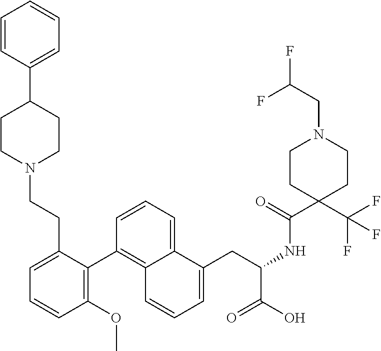 [SMILES: COc1cccc(CCN2CCC(c3ccccc3)CC2)c1-c1cccc2c(C[C@H](NC(=O)C3(C(F)(F)F)CCN(CC(F)F)CC3)C(=O)O)cccc12] |
| | m/z = 752.4 |
| 976 | 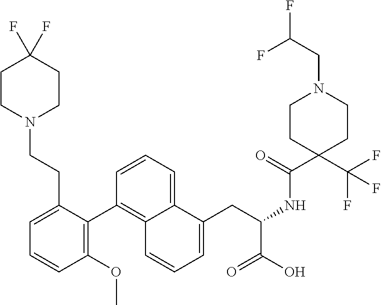 [SMILES: COc1cccc(CCN2CCC(F)(F)CC2)c1-c1cccc2c(C[C@H](NC(=O)C3(C(F)(F)F)CCN(CC(F)F)CC3)C(=O)O)cccc12] |
| | m/z = 712.2 |
| 977 | 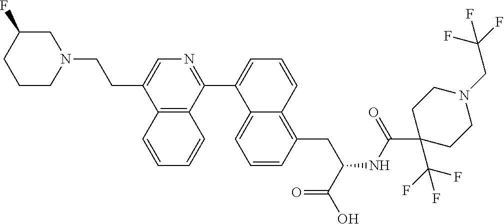 [SMILES: O=C(O)[C@H](Cc1cccc2c(-c3ncc(CCN4CCC[C@@H](F)C4)c4ccccc34)cccc12)NC(=O)C1(C(F)(F)F)CCN(CC(F)(F)F)CC1] |
| | m/z = 733.3 |
| 978 | 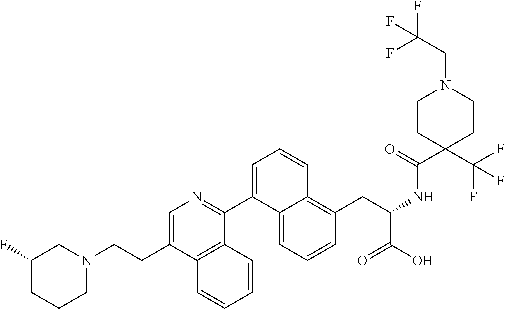 [SMILES: O=C(O)[C@H](Cc1cccc2c(-c3ncc(CCN4CCC[C@H](F)C4)c4ccccc34)cccc12)NC(=O)C1(C(F)(F)F)CCN(CC(F)(F)F)CC1] |
| | m/z = 733.3 |
| 979 | 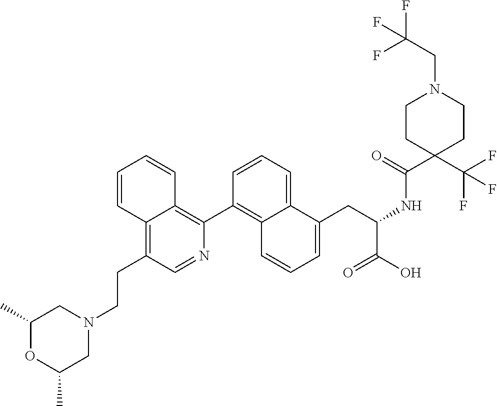 [SMILES: C[C@@H]1CN(CCc2cnc(-c3cccc4c(C[C@H](NC(=O)C5(C(F)(F)F)CCN(CC(F)(F)F)CC5)C(=O)O)cccc34)c3ccccc23)C[C@H](C)O1] |
| | m/z = 745.3 |
| 980 | 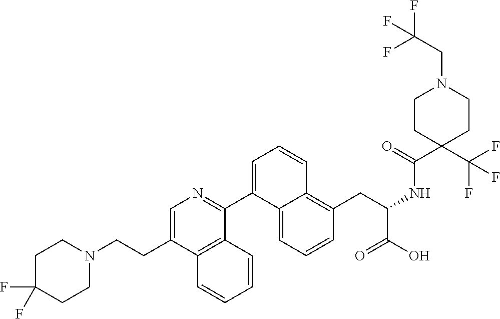 [SMILES: O=C(O)[C@H](Cc1cccc2c(-c3ncc(CCN4CCC(F)(F)CC4)c4ccccc34)cccc12)NC(=O)C1(C(F)(F)F)CCN(CC(F)(F)F)CC1] |
| | m/z = 751.2 |
| 981 | 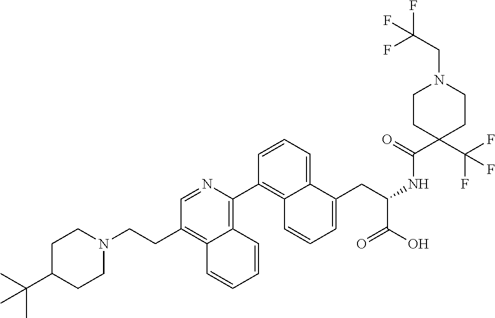 [SMILES: CC(C)(C)C1CCN(CCc2cnc(-c3cccc4c(C[C@H](NC(=O)C5(C(F)(F)F)CCN(CC(F)(F)F)CC5)C(=O)O)cccc34)c3ccccc23)CC1] |
| | m/z = 771.4 |
| 982 | 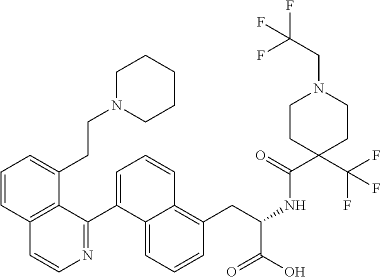 [SMILES: O=C(O)[C@H](Cc1cccc2c(-c3nccc4cccc(CCN5CCCCC5)c34)cccc12)NC(=O)C1(C(F)(F)F)CCN(CC(F)(F)F)CC1] |
| | m/z = 715.3 |
| 983 | 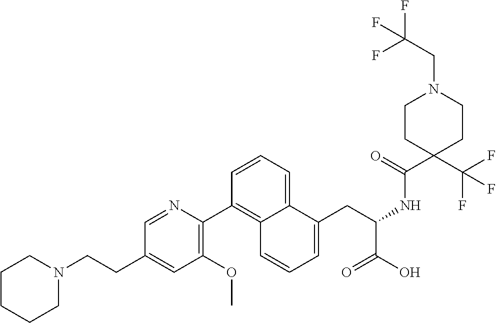 [SMILES: COc1cc(CCN2CCCCC2)cnc1-c1cccc2c(C[C@H](NC(=O)C3(C(F)(F)F)CCN(CC(F)(F)F)CC3)C(=O)O)cccc12] |
| | m/z = 695.3 |
| 984 | 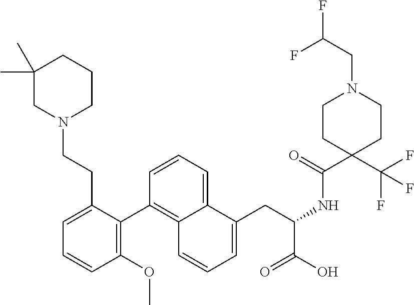 [SMILES: COc1cccc(CCN2CCCC(C)(C)C2)c1-c1cccc2c(C[C@H](NC(=O)C3(C(F)(F)F)CCN(CC(F)F)CC3)C(=O)O)cccc12] |
| | m/z = 704.4 |
| 985 | 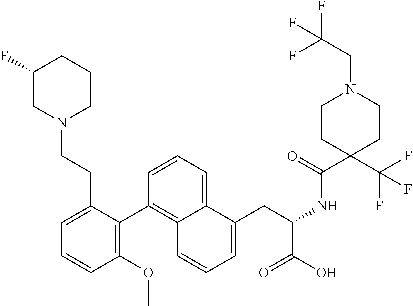 [SMILES: COc1cccc(CCN2CCC[C@@H](F)C2)c1-c1cccc2c(C[C@H](NC(=O)C3(C(F)(F)F)CCN(CC(F)(F)F)CC3)C(=O)O)cccc12] |
| | m/z = 712.2 |

*Denotes a racemic compound; **Denotes a single isomer with unknown absolute stereochemistry

### Example 43: General Scheme 2D

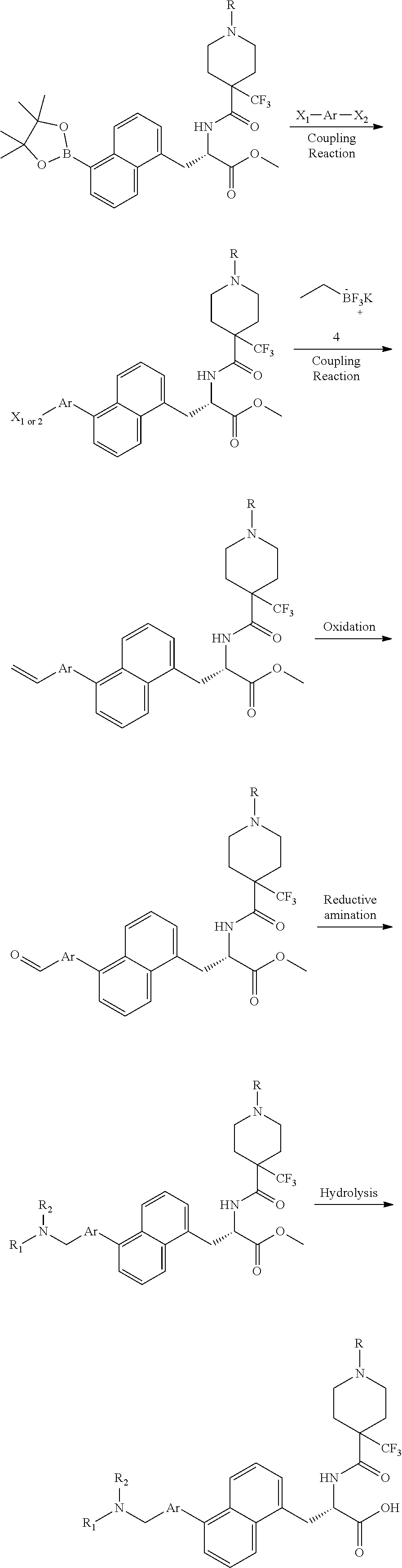

The proceeding compounds were synthesized according to the procedures described in General Scheme 2D using Common Intermediate 2 or Int2 as an intermediate.

### Synthesis of Compound 986

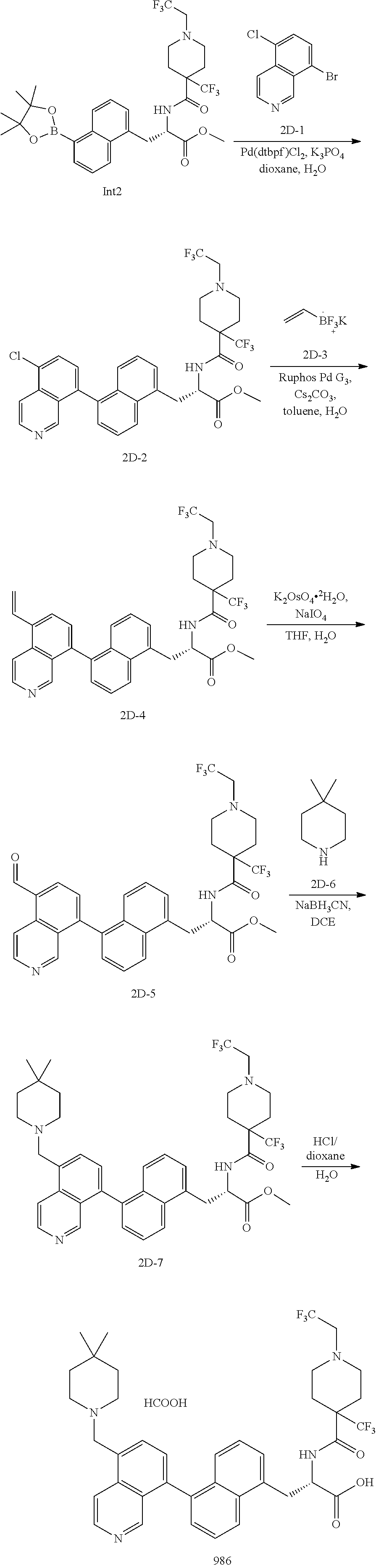

Step 1: A mixture of compound 2D-1 (590 mg, 2.43 mmol, 1.00 eq), compound Int2 (1.52 g 2.43 mmol, 1.00 eq), K₃PO₄ (1.55 g, 7.30 mmol, 3.00 eq), Pd(dtbpf)Cl₂ (158 mg, 243 μmol, 0.100 eq) in dioxane (12.0 mL) and H₂O (3.00 mL) was degassed and purged with N₂ for 3 times, and then the mixture was stirred at 25° C. for 4 hrs under N₂ atmosphere. The reaction mixture was diluted with H₂O 15.0 mL and extracted with ethyl acetate 30.0 mL (10.0 mL*3). The combined organic layers were concentrated under reduced pressure to give a residue. The residue was purified by Prep-TLC (SiO₂, Dichloromethane:Methanol=30:1, R_{f}=0.53). Compound 2D-2 (1.40 g, 2.15 mmol, 88.2% yield) was obtained as a yellow oil. LC-MS: (M+H)⁺: 652.3. ¹H NMR: (400 MHz, DMSO-d₆) δ 8.66 (t, J=5.6 Hz, 1H), 8.60 (dd, J₁=8.0 Hz, J₂=3.0 Hz, 1H), 8.54 (s, 1H), 8.30 (d, J=8.4 Hz, 1H), 8.16-8.07 (m, 2H), 7.81-7.74 (m, 1H), 7.68-7.57 (m, 2H), 7.41 (d, J=6.8 Hz, 1H), 7.30-7.25 (m, 1H), 7.11 (d, J=8.4 Hz, 1H), 4.97-4.76 (m, 1H), 3.85 (dd, J₁=14.4 Hz, J₂=4.0 Hz, 1H), 3.71 (d, J=3.2 Hz, 3H), 3.59-3.51 (m, 1H), 3.29 (s, 2H), 3.08-2.70 (m, 4H), 2.29-2.09 (m, 3H), 1.68-1.54 (m, 2H)

Step 2: A mixture of compound 2D-2 (1.40 g, 2.14 mmol, 1.00 eq), compound 2D-3 (142.3 mg, 10.70 mmol, 5.00 eq), Ruphos Pd G; (179 mg, 214 μmol, 0.100 eq), Cs₂CO₃ (2.10 g, 6.44 mmol, 3.00 eq) in toluene (32.0 mL) and H₂O (8.00 mL) was degassed and purged with N₂ for 3 times. The mixture was stirred at 80° C. for 2 hrs under N₂ atmosphere. The reaction mixture was diluted with H₂O 5.00 mL and extracted with ethyl acetate 15.0 mL (5.00 mL*3). The combined organic layers were concentrated under reduced pressure to give a residue. The residue was purified by Prep-TLC (SiO₂, Petroleum ether:Ethyl acetate=1:1, R_{f}=0.53). Compound 2D-4 (1.00 g, 1.55 mmol, 72.3% yield) was obtained as a yellow oil. LC-MS: (M+H)⁺: 644.3. ¹H NMR: (400 MHz, DMSO-d₆) 0.64-8.50 (m, 3H), 8.28 (d, J=8.8 Hz, 1H), 8.18 (d, J=6.0 Hz, 1H), 8.13-8.10 (m, 1H), 7.77 (t, J=8.0 Hz, 1H), 7.69-7.56 (m, 3H), 7.40 (br d, J=7.2 Hz, 1H), 7.32-7.22 (m, 1H), 7.16-7.06 (m, 1H), 6.06 (d, J=8.8 Hz, 1H), 5.63 (d, J=10.4 Hz, 1H), 4.95-4.75 (m, 1H), 3.91-3.80 (m, 1H), 3.75-3.68 (m, 3H), 3.61-3.48 (m, 1H), 3.20-3.15 (m, 2H), 3.07-2.95 (m, 2H), 2.18-2.09 (m, 2H), 2.06-1.86 (m, 2H), 1.82-1.64 (m, 2H).

Step 3: To a solution of compound 2D-4 (1.00 g, 1.55 mmol, 1.00 eq) in THF (12.0 mL) was added NaIO₄ (1.66 g, 7.77 mmol, 430 μL, 5.00 eq) at 0° C., then a solution of K₂OsO₄·2H₂O (171 mg, 466 μmol, 0.300 eq) in H₂O (6.00 mL) was added at 0° C. The mixture was stirred at 40° C. for 1 hr. The reaction mixture was diluted with H₂O 10.0 mL, extracted with ethyl acetate 30.0 mL (10.0 mL*3), and then quenched by addition sat. aq. Na₂SO₃ solution (50.0 mL) at 25° C. The combined organic layers were concentrated under reduced pressure to give a residue. The residue was purified by Prep-TLC (SiO₂, Dichloromethane:Methanol=40:1, R_{f}=0.27). Compound 2D-5 (700 mg, 1.08 mmol, 69.7% yield) was obtained as a yellow solid. LC-MS: (M+H)⁺: 646.3. ¹H NMR: (400 MHz, DMSO-d₆) δ 10.53 (s, 1H), 9.05-9.03 (m, 1H), 8.69 (t, J=6.0 Hz, 1H), 8.64-8.55 (m, 3H), 8.33 (d, J=8.8 Hz, 1H), 7.89 (dd, J₁=7.2 Hz, J₂=3.6 Hz, 1H), 7.83-7.75 (m, 1H), 7.63 (d, J=6.8 Hz, 1H), 7.42 (d, J=6.8 Hz, 1H), 7.34-7.23 (m, 1H), 7.09 (d, J=8.8 Hz, 1H), 4.97-4.77 (m, 1H), 3.88-3.86 (m, 1H), 3.72 (d, J=3.6 Hz, 3H), 3.63-3.46 (m, 1H), 3.17 (d, J=5.2 Hz, 2H), 3.07-2.95 (m, 1H), 2.94-2.84 (m, 1H), 2.29-2.10 (m, 2H), 2.07-1.87 (m, 2H), 1.64-1.55 (m, 2H)

Step 4: To a solution of compound 2D-5 (31.5 mg, 278 μmol, 1.50 eq) was added compound 2D-6 (120 mg, 185 μmol, 1.00 eq) in DCE (2.00 mL). The mixture was stirred at 25° C. for 8 hrs. NaBH₃CN (23.3 mg, 371 μmol, 2.00 eq) was added, and the mixture was stirred at 25° C. for 2 hrs. The reaction mixture was diluted with H₂O 5.00 mL, and extracted with ethyl acetate 15.0 mL (5.00 mL*3). The combined organic layers concentrated under reduced pressure to give a residue. The residue was purified by Prep-TLC (SiO₂, Dichloromethane:Methanol=20:1, R_{f}=0.35). Compound 2D-7 (60.0 mg, 80.7 μmol, 43.4% yield) was obtained as a yellow oil. LC-MS: (M+H)⁺: 743.3.

Step 5: To a solution of compound 2D-7 (60.0 mg, 80.7 μmol, 1.00 eq) and H₂O (2.00 mL) was added HCl/dioxane (4.00 M, 2.00 mL, 99.0 eq). The mixture was stirred at 60° C. for 2 hrs. The reaction mixture was concentrated under reduced pressure to give a residue. The residue was purified by Prep-HPLC (column: Phenomenex luna C18 150*25 mm*10 um; mobile phase: [water (FA)-ACN]; gradient: 22%-52% B over 10 min). Compound 986 (28.25 mg, 34.6 μmol, 42.9% yield, 95.0% purity, FA) was obtained as a white solid. LC-MS: (M+H)⁺: 729.4. ¹H NMR: (400 MHz, DMSO-d₆). δ 13.11-12.65 (m, 1H), 8.54-8.47 (m, 2H), 8.45 (d, J=8.0 Hz, 1H), 8.23-8.20 (m, 1H), 8.21 (d, J=6.0 Hz, 1H), 7.83 (dd, J₁=3.0 Hz, J₂=7.2 Hz, 1H), 7.78-7.74 (m, 1H), 7.61-7.52 (m, 2H), 7.42 (d, J=6.8 Hz, 1H), 7.28-7.23 (m, 1H), 7.08 (d, J=8.4 Hz, 1H), 4.98-4.66 (m, 1H), 4.10-3.93 (m, 2H), 3.91-3.79 (m, 1H), 3.53-3.46 (m, 1H), 3.01-2.69 (m, 4H), 2.31-2.08 (m, 3H), 2.06-1.79 (m, 3H), 1.70-1.60 (m, 2H), 1.64-1.58 (m, 2H), 1.37-1.34 (m, 4H), 0.93 (s, 6H).

The compounds set forth in Table 23, were prepared using the synthetic procedures of Example 43, or analogous procedures as provided herein.

TABLE 23
Structure and Spectroscopic data for Compounds 987 to 989

| No. | Structure |
|---|---|
| 987 | 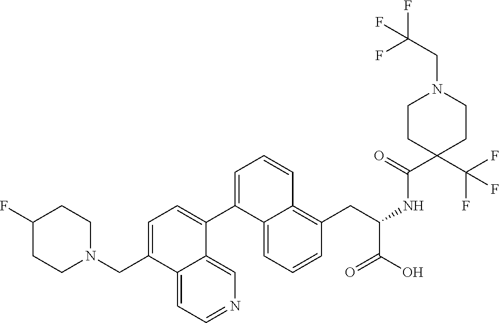 [SMILES: O=C(O)[C@H](Cc1cccc2c(-c3ccc(CN4CCC(F)CC4)c4ccncc34)cccc12)NC(=O)C1(C(F)(F)F)CCN(CC(F)(F)F)CC1] |
| | m/z = 719.3 |
| 988 | 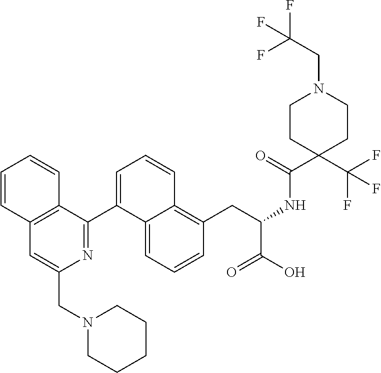 [SMILES: O=C(O)[C@H](Cc1cccc2c(-c3nc(CN4CCCCC4)cc4ccccc34)cccc12)NC(=O)C1(C(F)(F)F)CCN(CC(F)(F)F)CC1] |
| | m/z = 701.2 |
| 989* | 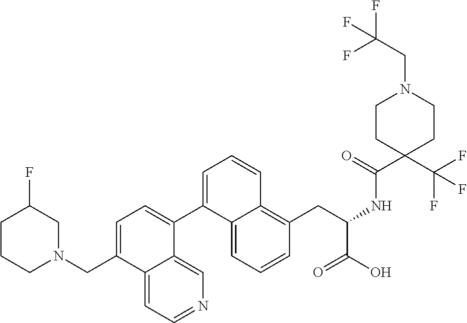 [SMILES: O=C(O)[C@H](Cc1cccc2c(-c3ccc(CN4CCCC(F)C4)c4ccncc34)cccc12)NC(=O)C1(C(F)(F)F)CCN(CC(F)(F)F)CC1] |
| | m/z = 719.4 |

*Denotes a racemic compound

### Example 44: Synthesis of Compound 990

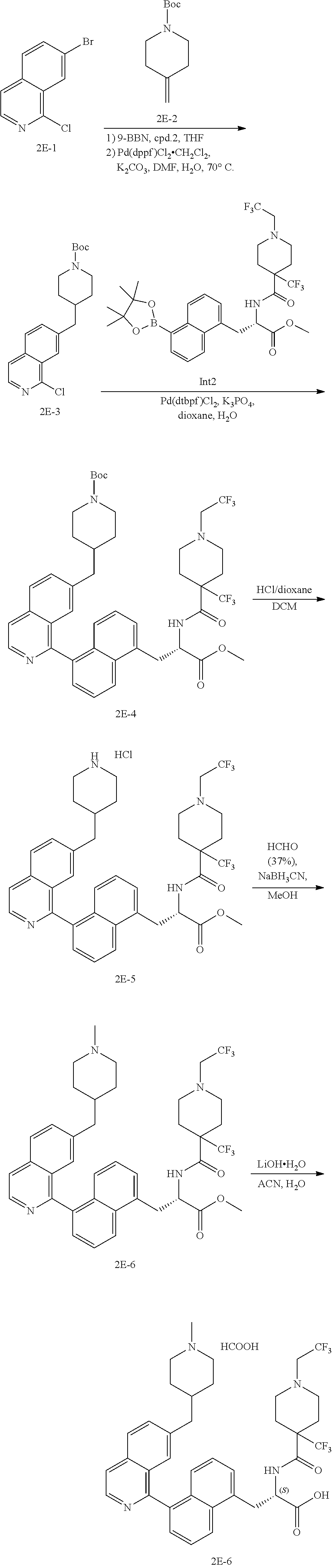

Step 1: To a solution of compound 2E-1 (244 mg, 1.24 mmol, 1.00 eq) in THF (4.00 mL) was added 9-BBN (0.500 M, 2.47 mL, 1.00 eq), the resulting solution was refluxed at 70° C. for 1 hr, after cooled to 20° C., the solution was added a mixture of compound 2E-2 (300 mg, 1.24 mmol, 1.00 eq), Pd(dppf)Cl₂·CH₂Cl₂ (101 mg, 124 μmol, 0.100 eq), K₂CO₃ (205 mg, 1.48 mmol, 1.20 eq), DMF (4.00 mL) and H₂O (1.00 mL) under N₂ atmosphere. The mixture was stirred at 70° C. for 12 hrs. The reaction mixture was quenched by addition saturated NH₄Cl solution 50.0 mL at 0° C., and then diluted with water 80.0 mL and extracted with EtOAc 300 mL (100 mL*3). The combined organic layers were washed with brine 100 mL (100 mL*1), dried over Na₂SO₄, filtered, and concentrated under reduced pressure to give a residue. The residue was purified by column chromatography (SiO₂, Petroleum ether:Ethyl acetate=1:0 to 3:1) and Prep-TLC (SiO₂, SiO₂, Petroleum ether:Ethyl acetate=3:1). Compound 2E-3 (90.0 mg, 249 μmol, 20.2% yield) was obtained as yellow oil. LC-MS: (M+H)⁺: 361.1. ¹H NMR: (400 MHz, CDCl₃) δ 8.25 (d, J=5.6 Hz, 1H), 8.08 (s, 1H), 7.79 (d, J=8.4 Hz, 1H), 7.63-7.53 (m, 2H), 4.20-4.01 (m, 2H), 2.79 (d, J=7.2 Hz, 2H), 2.66 (t, J=12.4 Hz, 2H), 1.90-1.75 (m, 2H), 1.56-1.48 (m, 1H), 1.46 (s, 9H), 1.29-1.21 (m, 2H)

Step 2: A solution of compound 2E-3 (80.0 mg, 222 μmol, 1.00), compound Int2 (139 mg, 222 μmol, 1.00 eq), Pd(dtbpf)Cl₂ (14.5 mg, 22.2 μmol, 0.100 eq) and K₃PO₄ (94.1 mg, 443 μmol, 2.00 eq) in dioxane (4.00 mL), H₂O (1.00 mL) was degassed and purged with N₂ for 3 times. The mixture was stirred at 20° C. for 1 hr under N₂ atmosphere. The reaction mixture was diluted with water 30.0 mL, and extracted with ethyl acetate 90.0 mL (30.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Petroleum ether:Ethyl acetate=1:1). Compound 2E-4 (130 mg, 158 μmol, 71.3% yield, 99.0% purity) was obtained as yellow oil. LC-MS: (M+H)⁺: 815.4

Step 3: To a solution of compound 2E-4 (115 mg, 140 μmol, 1.00 eq) in DCM (3.00 mL) was added HCl/dioxane (4.00 M, 1.00 mL, 28.6 eq), the mixture was stirred at 20° C. for 1.5 hrs. The reaction mixture was concentrated under reduced pressure to give a residue. Compound 2E-5 (90.0 mg, 120 μmol, 85.8% yield, HCl) was obtained as yellow oil. LC-MS: (M+H)⁺: 715.4

Step 4: To a solution of compound 2E-5 (70.0 mg, 93.2 μmol, 1.00 eq, HCl) in MeOH (3.00 mL) was added HCHO (15.1 mg, 186 μmol, 13.9 μL, 37.0% purity, 2.00 eq) and NaBH₃CN (11.7 mg, 186 μmol, 2.00 eq), the mixture was stirred at 20° C. for 1 hr. The reaction mixture was concentrated under reduced pressure to give a residue. Compound 2E-6 (65.0 mg, 89.2 μmol, 95.7% yield) was obtained as yellow oil. LC-MS: (M+H)⁺: 729.2

Step 5: To a solution of compound 2E-6 (60.0 mg, 82.3 μmol, 1.00 eq) in ACN (2.00 mL) was added a solution of LiOH·H₂O (5.18 mg, 124 μmol, 1.50 eq) in H₂O (0.500 mL), the mixture was stirred at 20° C. for 1 hr. The residue was purified by Prep-HPLC (column: Welch Ultimate C18 150*25 mm*5 um; mobile phase: [water (FA)-ACN]; gradient: 8%-38% B over 10 min). Compound 990 (23.59 mg, 33.0 μmol, 40.1% yield, 100% purity, FA) was obtained as white solid. LC-MS: (M+H)⁺: 715.4. ¹H NMR: (400 MHz, DMSO-d₆) δ 8.59 (d, J=6.4 Hz, 1H), 8.50-8.28 (m, 1H), 8.10-7.95 (m, 2H), 7.94-7.86 (m, 1H), 7.81-7.65 (m, 1H), 7.64-7.53 (m, 2H), 7.47-7.33 (m, 1H), 7.22-7.10 (m, 1H), 7.03-6.76 (m, 2H), 4.70-4.58 (m, 1H), 3.84-3.48 (m, 2H), 3.09-3.02 (m, 1H), 3.00-2.89 (m, 2H), 2.86-2.67 (m, 3H), 2.65-2.56 (m, 1H), 2.42-2.32 (m, 3H), 2.31-2.05 (m, 6H), 2.01-1.33 (m, 5H), 1.27-0.60 (m, 3H)

### Example 45: Synthesis of Compound 991

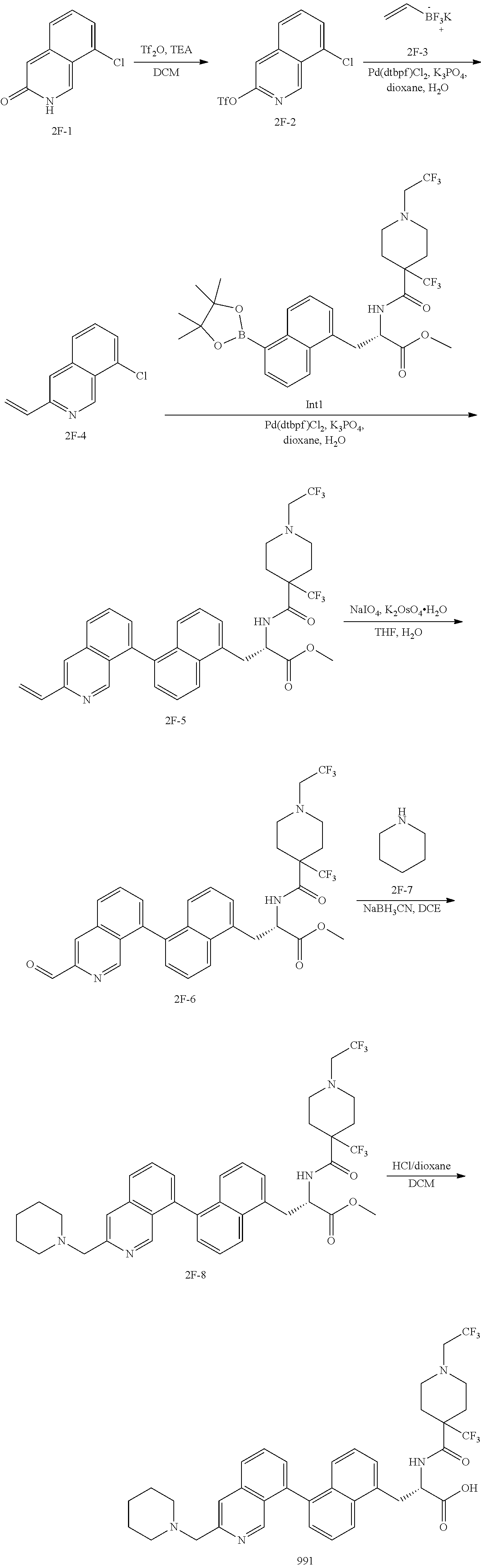

Step 1: To a solution of compound 2F-1 (1.00 g, 5.57 mmol, 1.00 eq) in DCM (20.0 mL) was added Tf₂O (3.14 g, 11.1 mmol, 1.84 mL, 2.00 eq) and TEA (1.69 g, 16.7 mmol, 2.32 mL, 3.00 eq) at 0° C. The mixture was stirred at 20° C. for 2 hrs. The reaction mixture was diluted with sat. aq. NaHCO₃20.0 mL and extracted with DCM 60.0 mL (20.0 mL*3). The combined organic layers were washed with brine 60.0 mL (20.0 mL*3), dried over Na₂SO₄, filtered, and concentrated under reduced pressure to give a residue. The residue was purified by Prep-TLC (SiO₂, PE:EtOAc=3:1). Compound 2F-2 (1.00 g, 3.21 mmol, 57.6% yield) was obtained as a yellow oil. LC-MS: (M-55)⁺: 311.9.

Step 2: To a solution of compound 2F-2 (1.00 g, 3.21 mmol, 1.00 eq) in dioxane (20.0 mL) and H₂O (5.00 mL) was added compound 2F-3 (2.15 g, 16.0 mmol, 5.00 eq), Pd(dtbpf)Cl₂ (209 mg, 321 μmol, 0.100 eq) and K₃PO₄ (2.04 g, 9.63 mmol, 3.00 eq). The mixture was stirred at 40° C. for 2 hrs. The reaction mixture was diluted with water 20.0 mL, and extracted with EtOAc 60.0 mL (20.0 mL*3). The combined organic layers were washed with brine 60.0 mL (20.0 mL*3), dried over Na₂SO₄, filtered, and concentrated under reduced pressure to give a residue. The residue was purified by column chromatography (SiO₂, PE:EtOAc=1:0 to 3:1). Compound 2F-4 (400 mg, 2.11 mmol, 65.7% yield) was obtained as a yellow solid. LC-MS (M+H)⁺: 581.5. ¹H NMR: (400 MHz, CDCl₃) δ 9.64 (s, 1H), 7.71 (dd, J₁=6.4 Hz, J₂=2.8 Hz, 1H), 7.58-7.56 (m, 3H), 6.96 (dd, J₁=17.2 Hz, J₂=10.8 Hz, 1H), 6.47-6.42 (m, 1H), 5.56 (d, J=10.8 Hz, 1H).

Step 3: Compound 2F-4 (150 mg, 791 μmol, 1.00 eq), compound Int1 (493 mg, 791 μmol, 1.00 eq), K₃PO₄ (504 mg, 2.37 mmol, 3.00 eq) and Pd(dtbpf)Cl₂ (51.5 mg, 79.1 μmol, 0.100 eq) were taken up into a microwave tube in dioxane (4.00 mL) and H₂O (1.00 mL). The sealed tube was heated at 60° C. for 120 min under microwave. The reaction mixture was diluted with water 15.0 mL, and extracted with EtOAc 60.0 mL (20.0 mL*3). The combined organic layers were washed with brine 40.0 mL (20.0 mL*2), dried over Na₂SO₄, filtered, and concentrated under reduced pressure to give a residue. The residue was purified by Prep-TLC (SiO₂, PE:EtOAc=3:1). Compound 2F-5 (150 mg, 233 μmol, 29.5% yield) was obtained as a yellow oil. LC-MS: (M+H)⁺: 644.1. ¹H NMR: (400 MHz, CDCl₃) δ 8.70 (d, J=12.4 Hz, 1H), 8.26 (t, J=8.2 Hz, 1H), 7.90 (d, J=8.4 Hz, 1H), 7.77 (t, J=7.2 Hz, 1H), 7.70 (dd, J₁=8.4 Hz, J₂=7.2 Hz, 1H), 7.65 (s, 1H), 7.55-7.53 (m, 2H), 7.34-7.29 (m, 2H), 7.25-7.21 (m, 1H), 6.98-6.90 (m, 1H), 6.40 (d, J=7.2 Hz, 1H), 6.35-6.31 (m, 1H), 5.49-5.46 (m, 1H), 5.12-5.08 (m, 1H), 3.72-3.64 (m, 5H), 2.91-2.80 (m, 4H), 2.38-2.36 (m, 1H), 2.23-2.07 (m, 3H), 1.99-1.91 (m, 2H).

Step 4: To a solution of compound 2F-5 (150 mg, 233.06 μmol, 1 eq) in THF (3.00 mL) and H₂O) (3.00 mL) was added NaIO₄ (149 mg, 699 μmol, 38.7 μL, 3.00 eq) and K₂OsO₄·2H₂O (12.9 mg, 35.0 μmol, 0.150 eq) at 0° C. The mixture was stirred at 20° C. for 3 hrs. The reaction mixture was diluted with water 15.0 mL and extracted with EtOAc 45.0 mL (15.0 mL*3). The combined organic layers were washed with brine 30.0 ml. (15.0 ml.*2), dried over Na 2SO₄, filtered and concentrated under reduced pressure to give a residue. The residue was purified by Prep-TLC (SiO₂, DCM:MeOH=10:1). Compound 2F-6 (90.0 mg, 139 μmol, 59.8% yield) was obtained as a yellow solid. LC-MS: (M+H)⁺: 646.1

Step 5: To a solution of compound 2F-6 (90.0 mg, 139 μmol, 1.00 eq) in DCE (2.00 mL) was added compound 2F-7 (35.6 mg, 418 μmol, 41.3 μL, 3.00 eq). The mixture was stirred at 20° C. for 2 hrs. And then the mixture was added NaBH₃CN (13.1 mg, 209 μmol, 1.50 eq) and stirred at 20° C. for 3 hrs. The reaction mixture was diluted with water 15.0 mL and extracted with 45.0 mL (15.0 mL*3). The combined organic layers were washed with brine 45.0 mL (15.0 mL*3), dried over Na₂SO₄, filtered, and concentrated under reduced pressure to give a residue. The residue was purified by Prep-TLC (SiO₂, DCM:MeOH=10:1). Compound 2F-8 (40.0 mg, 56.0 μmol, 40.1% yield) was obtained as a yellow solid. LC-MS: (M+H)⁺: 715.3. ¹H NMR: (400 MHz, CDCl₃) δ 8.71 (d, J=10.8 Hz, 1H), 8.25 (t, J=9.6 Hz, 1H), 7.93 (d, J=8.4 Hz, 1H), 7.89-7.82 (m, 1H), 7.81-7.77 (m, 1H), 7.71-7.68 (m, 1H), 7.54-7.53 (m, 2H), 7.34 (d, J=8.0 Hz, 1H), 7.31-7.28 (m, 1H), 7.24-7.20 (m, 1H), 6.39 (d, J=6.4 Hz, 1H), 5.13-5.07 (m, 1H), 3.82 (br s, 2H), 3.71-3.50 (m, 7H), 2.90-2.80 (m, 4H), 2.58 (br s, 3H), 2.40-2.33 (m, 1H), 2.26-2.17 (m, 1H), 2.11-2.08 (m, 2H), 2.01-1.88 (m, 3H), 1.68 (br s, 4H).

Step 6: To a solution of compound 2F-8 (35.0 mg, 49.0 μmol, 1.00 eq) in H₂O (1.00 mL) was added HCl/dioxane (4.00 M, 2.00 mL). The mixture was stirred at 60° C. for 2 hrs. The reaction mixture was concentrated under reduced pressure to give a residue. The residue was purified by Prep-HPLC (column: Welch Xtimate C18 150*25 mm*5 um; mobile phase: [water (FA)-ACN]; gradient: 18%-48% B over 10 min). Compound 991 (29.92 mg, 40.1 μmol, 81.8% yield, 100% purity, HCOOH) was obtained as a white solid. LC-MS: (M+H)⁺: 701.3. ¹H NMR: (400 MHz, DMSO-d₆) δ 8.46 (s, 1H), 8.42 (d, J=8.4 Hz, 1H), 8.32 (d, J=8.4 Hz, 1H), 8.10 (d, J=8.4 Hz, 1H), 7.92 (s, 1H), 7.90-7.85 (m, 1H), 7.78-7.74 (m, 1H), 7.58-7.55 (m, 2H), 7.42 (d, J=7.2 Hz, 1H), 7.29-7.24 (m, 1H), 7.11 (d, J=8.4 Hz, 1H), 4.88-4.74 (m, 1H), 3.88-3.83 (m, 1H), 7.70 d, J=6.4 Hz, 2H), 3.53-3.47 (m, 1H), 2.97-2.89 (m, 1H), 2.87-2.61 (m, 4H), 2.47 (br s, 2H), 2.33-2.13 (m, 3H), 1.99-1.80 (m, 2H), 1.63-1.53 (m, 6H), 1.42-1.41 (m, 2H).

### Example 46: Synthesis of Compound 992

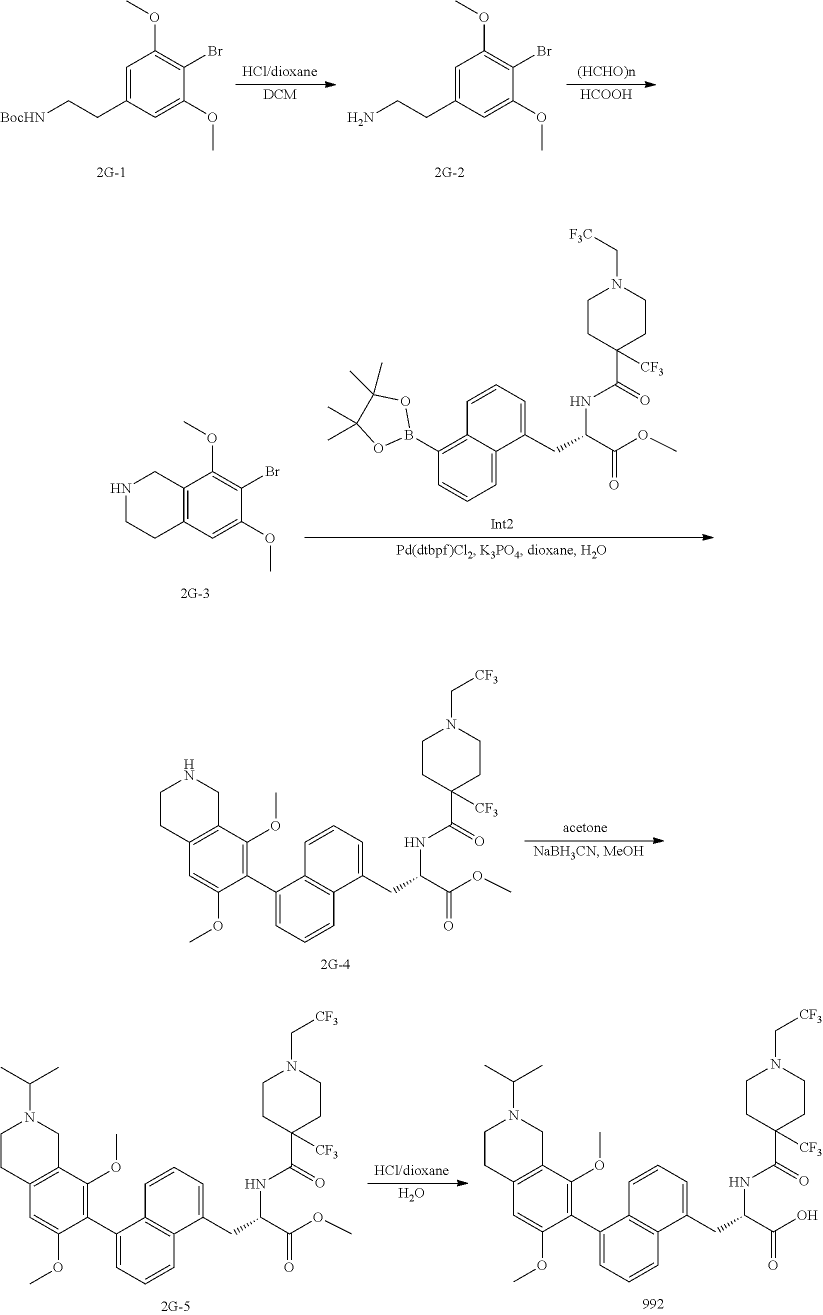

Step 1: To a solution of compound 2G-1 (2.00 g, 5.55 mmol, 1.00 eq) in DCM (10.0 mL) was added HCl/dioxane (4 M, 10.0 mL, 7.20 eq). The mixture was stirred at 25° C. for 1 hr. The reaction mixture was concentrated under reduced pressure to give a residue. Then the residue was dissolved with H₂O (20.0 ml). The pH of aqueous layer was adjusted to 9 with sat. NaHCO₃ aq., and then extracted with ethyl acetate (50.0 mL*3). The combined organic layers were washed with brine (50.0 mL*2), concentrated under reduced pressure to give a residue. Compound 2G-2 (1.50 g, crude) was obtained as yellow oil.

Step 2: To a solution of compound 2G-2 (1.30 g, 5.00 mmol, 1.00 eq) in HCOOH (15.0 mL) was added (HCHO) n (350 mg, 1.54 mmol). The mixture was stirred at 60° C. for 0.5 hr. Then the residue was dissolved with H₂O (10.0 ml). The pH of aqueous layer was adjusted to 8 with sat. NaOH aq., and extracted with ethyl acetate (50.0 mL*3). The combined organic layers were washed with brine (50.0 mL*2), concentrated under reduced pressure to give a residue. The residue was purified by Prep-HPLC (column: Welch Ultimate XB-SiOH 250*70*10 um; mobile phase: [Hexane-EtOH]; gradient: 20%-90% B over 5.5 min). Compound 2G-3 (400 mg, 1.47 mmol, 29.4% yield) was obtained as a brown solid. LC-MS: (M+H)⁺: 273.8. ¹H NMR: (400 MHz, DMSO-d₆) δ 6.63 (s, 1H), 3.81-3.76 (m, 5H), 3.69 (s, 3H), 2.89 (t, J=5.6 Hz, 2H), 2.63 (br t, J=5.6 Hz, 2H)

Step 3: A mixture of compound 2G-3 (350 mg, 1.29 mmol, 1.00 eq), compound Int2 (806 mg, 1.29 mmol, 1.00 eq), K₃PO₄ (819 mg, 3.86 mmol, 3.00 eq), Pd(dtbpf)Cl₂ (83.8 mg, 129 μmol, 0.100 eq) in dioxane (6.00 mL) and H₂O (2.00 mL) was degassed and purged with N₂ for 3 times, and then the mixture was stirred at 25° C. for 1 hr under N₂ atmosphere. Water 20 mL was added into reaction mixture, diluted with ethyl acetate 10.0 mL, and extracted with ethyl acetate (20.0 mL*3). The combined organic layers were washed with brine (20.0 mL*4), dried over Na 2SO₄ filtered and concentrated under reduced pressure to give a residue. The residue was purified by Prep-HPLC (column: Welch Xtimate XB_C 18 20-40 μm; 120 A; mobile phase: [H₂O (HCl)+ACN]; gradient: 15-45% 25 min; 50% 10 min). Compound 2G-4 (350 mg, 513 μmol, 39.9% yield) was obtained as a white solid. LC-MS: (M+H)⁺: 682.0. ¹H NMR: (400 MHz, DMSO-d₆) δ 8.59 (br t, J=7.6 Hz, 1H), 8.08 (br d, J=8.0 Hz, 1H), 7.62 (br t, J=8.0 Hz, 1H), 7.34-7.27 (m, 4H), 6.68 (brs, 1H), 4.86-4.75 (m, 1H), 3.83-3.75 (m, 3H), 3.69 (s, 3H), 3.53-3.48 (m, 4H), 3.05 (s, 3H), 3.02-2.89 (m, 4H), 2.79-2.68 (m, 4H), 2.31-2.25 (m, 2H), 2.14-2.03 (m, 1H), 1.95-1.85 (m, 1H), 1.65-1.55 (m, 2H), 1.39-1.23 (m, 1H)

Step 4: To a solution of compound 2G-4 (100 mg, 147 μmol, 1.00 eq) in MeOH (2.00 mL) was added acetone (17.0 mg, 293 μmol, 21.6 μL, 2.00 eq) stirred for 1 hr, then added NaBH₃CN (27.6 mg, 440 μmol, 3.00 eq) at 25° C. The mixture was stirred at 25° C. for 1 hr. Water 20.0 mL was added into reaction mixture, diluted with ethyl acetate 20.0 mL, and extracted with ethyl acetate (20.0 mL*3). The combined organic layers were washed with brine (20.0 mL*4), dried over Na₂SO₄ filtered, and concentrated under reduced pressure to give a residue. Compound 2G-5 (90.0 mg, crude) was obtained as a yellow solid. LC-MS: (M+H)⁺: 724.1. ¹H NMR (400 MHz, DMSO-d₆) δ 8.59 (br t, J=8.4 Hz, 1H), 8.09 (br d, J=7.2 Hz, 1H), 7.63 (br t, J=8.0 Hz, 1H), 7.35-7.34 (m, 2H), 7.30-7.24 (m, 2H), 6.75-6.67 (m, 1H), 4.88-4.75 (m, 1H), 3.81-3.72 (m, 2H), 3.69 (s, 3H), 3.57-3.47 (m, 5H), 3.07-3.04 (m, 3H), 3.02-2.88 (m, 5H), 2.79-2.67 (m, 4H), 2.33-2.12 (m, 3H), 1.94-1.83 (m, 1H), 1.65-1.55 (m, 2H), 1.34-1.23 (m, 2H), 1.14-1.02 (m, 4H)

Step 5: To a solution of compound 2G-5 (70.0 mg, 96.7 μmol, 1.00 eq) in HCl/dioxane (4 M, 2.00 mL, 82.7 eq) was added H₂O (1.00 mL). The mixture was stirred at 25° C. for 1 hr. The reaction mixture was concentrated under reduced pressure to give a residue. The residue was purified by Prep-HPLC (column: Welch Xtimate C 18 150*25 mm*5 um; mobile phase: [water (FA)-ACN]; gradient: 20%-50% B over 0 min) to give residue, then further purified by Prep-HPLC (column: Phenomenex C 18 150*25 mm*10 um; mobile phase: [water (NH₄HCO₃)-ACN]; gradient: 25%-55% B over 14 min). Compound 992 (33.91 mg, 47.4 μmol, 49.0% yield, 99.2% purity) was obtained as a white solid. LC-MS: (M+H)⁺: 710.2. ¹H NMR: (400 MHz, DMSO-d₆) δ 8.41-8.36 (m, 1H), 8.14 (br d, J=8.4 Hz, 1H), 7.62 (t, J=7.6 Hz, 1H), 7.35-7.33 (m, 2H), 7.28-7.23 (m, 2H), 6.71-6.69 (m, 1H), 4.79-4.68 (m, 1H), 3.79-3.75 (m, 2H), 3.61 (br s, 2H), 3.53 (brd, J=2.0 Hz, 3H), 3.49-3.40 (m, 2H), 3.07 (s, 3H), 2.93-2.87 (m, 4H), 2.74-2.73 (m, 1H), 2.31-2.15 (m, 4H), 1.98-1.82 (m, 2H), 1.64-1.53 (m, 2H), 1.08 (br d, J=5.6 Hz, 6H)

### Example 47: Synthesis of Compound 993

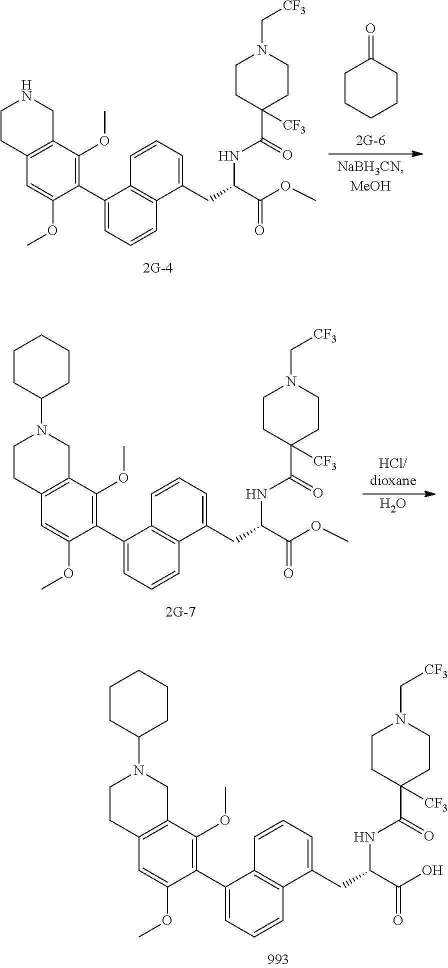 [SMILES: COC(=O)[C@H](Cc1cccc2c(-c3c(OC)cc4c(c3OC)CNCC4)cccc12)NC(=O)C1(C(F)(F)F)CCN(CC(F)(F)F)CC1 ; O=C1CCCCC1 ; COC(=O)[C@H](Cc1cccc2c(-c3c(OC)cc4c(c3OC)CN(C3CCCCC3)CC4)cccc12)NC(=O)C1(C(F)(F)F)CCN(CC(F)(F)F)CC1 ; O ; COc1cc2c(c(OC)c1-c1cccc3c(C[C@H](NC(=O)C4(C(F)(F)F)CCN(CC(F)(F)F)CC4)C(=O)O)cccc13)CN(C1CCCCC1)CC2]

Step 1: To a solution of compound 2G-4 (100 mg, 146 μmol, 1.00 eq) in MeOH (3.00 mL) was added compound 2G-6 (28.8 mg, 293 μmol, 30.4 μL, 2.00 eq) and stirred for 1 hr, then added NaBH₃CN (27.6 mg, 440 μmol, 3.00 eq). The mixture was stirred at 25° C. for 1 hr. The reaction mixture was addition water 20.0 mL, diluted with ethyl acetate 20.0 mL and extracted with ethyl acetate (20.0 mL*3). The combined organic layers were washed with brine (20.0 mL*4), dried over Na₂SO₄ filtered and concentrated under reduced pressure to give a residue. Compound 2G-7 (100 mg, crude) was obtained as a yellow solid. LC-MS: (M+H)⁺: 764.2. ¹H NMR: (400 MHz, DMSO-d₆) δ 8.59 (brt, J=8.4 Hz, 1H), 8.08 (brd, J=8.8 Hz, 1H), 7.62 (t, J=8.0 Hz, 1H), 7.35-7.32 (m, 2H), 7.30-7.26 (m, 2H), 6.67 (d, J=2.8 Hz, 1H), 4.87-4.74 (m, 1H), 3.81-3.74 (m, 1H), 3.69 (d, J=1.2 Hz, 3H), 3.57-3.51 (m, 4H), 3.40-3.36 (m, 1H), 3.10-3.04 (m, 3H), 3.0-2.84 (m, 4H), 2.81-2.70 (m, 4H), 1.94-1.88 (m, 3H), 1.58-1.52 (m, 3H), 1.42-1.32 (m, 3H), 1.29-1.21 (m, 5H), 1.18-1.08 (m, 4H).

To a solution of compound 2G-7 (70.0 mg, 91.6 μmol, 1.00 eq) in HCl/dioxane (4 M, 2.00 mL, 87.2 eq) was added H₂O (1.00 mL). The mixture was stirred at 60° C. for 1 hr. The reaction mixture was concentrated under reduced pressure to give a residue. The residue was purified by Prep-HPLC (column: Welch Xtimate C 18 150*25 mm*5 μm; mobile phase: [water (FA)-ACN]; gradient: 20%-50% B over 10 min) to give residue, then further purified by Prep-HPLC (column: Phenomenex C 18 150*25 mm*10 um; mobile phase: [water (NH₄HCO₃)-ACN]; gradient: 33%-63% B over 14 min). Compound 993 (37.25 mg, 49.3 μmol, 53.8% yield, 99.3% purity) was obtained as a white solid. LC-MS: (M+H)⁺: 750.2. ¹H NMR: (400 MHz, DMSO-d₆) δ 8.41-8.36 (m, 1H), 8.13 (br d, J=8.6 Hz, 1H), 7.61 (brt, J=7.6 Hz, 1H), 7.35-7.32 (m, 2H), 7.28-7.24 (m, 2H), 6.68 (brd, J=2.8 Hz, 1H), 4.77-4.68 (m, 1H), 3.77 (br d, J=14.0 Hz, 1H), 3.72-3.61 (m, 2H), 3.52 (s, 3H), 3.44 (br d, J=14.0 Hz, 1H), 3.06 (s, 3H), 2.95-2.85 (m, 5H), 2.80-2.73 (m, 2H), 2.24 (br d, J=14.0 Hz, 4H), 2.00-1.94 (m, 1H), 1.86 (br s, 2H), 1.77 (br d, J=9.2 Hz, 2H), 1.59 (br d, J=8.0 Hz, 3H), 1.33-1.10 (m, 6H).

### Example 48: Synthesis of Compound 994

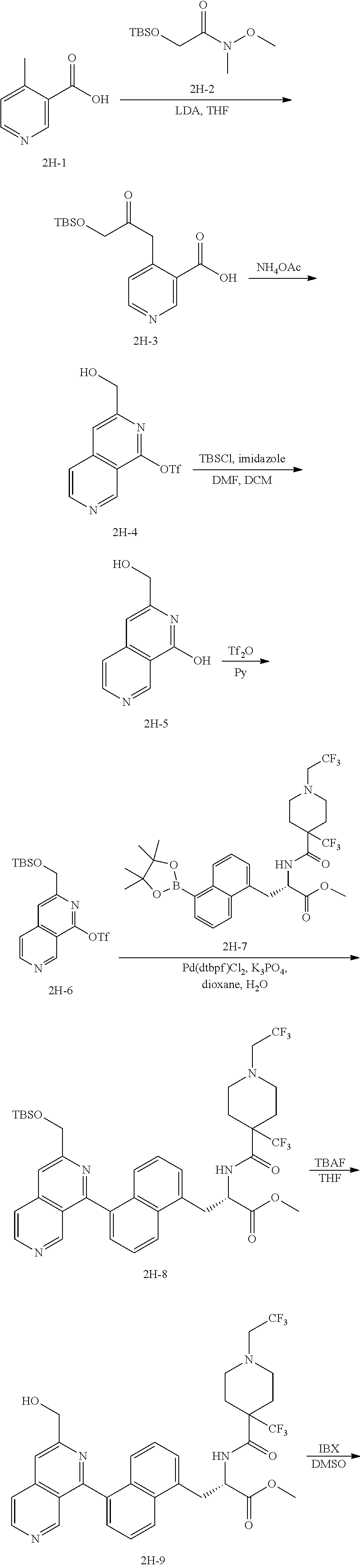

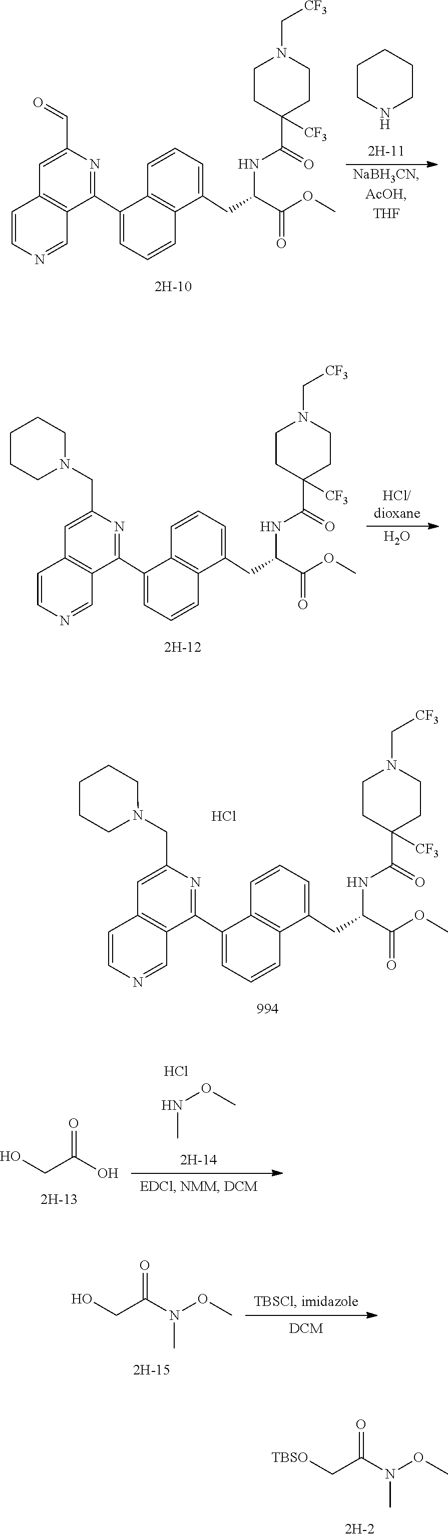

Step 1: To a solution of compound 2H-13 (20.0 g, 262 mmol, 16.0 mL, 1.00 eq) in DCM (200 mL) was added NMM (26.6 g, 262 mmol, 28.9 mL, 1.00 eq), compound 2H-14 (25.6 g, 262 mmol, 1.00 eq) and EDCI (50.4 g, 262 mmol, 1.00 eq) at 0° C. The mixture was stirred at 25° C. for 10.0 hrs. HCl (60.0 mL, 2.00 M) was added to the mixture at 0° C., then extracted with DCM (200 mL*5). The combined organic phase was dried over Na₂SO₄ and concentrated to give a residue. Compound 2H-15 (27.0 g, 226 mmol, 86.1% yield) was obtained as colorless oil. ¹H NMR: (400 MHz, CDCl₃) δ 4.28 (s, 2H), 3.68 (s, 3H), 3.23 (s, 3H)

Step 2: To a solution of compound 2H-15 (25.0 g, 209 mmol, 1.00 eq) in DCM (250 mL) was added TBSCl (34.8 g, 230 mmol, 28.4 mL, 1.10 eq) and imidazole (17.1 g, 251 mmol, 1.20 eq) at 0° C., then the mixture was stirred at 25° C. for 8 hrs. The mixture was filtered, and the filtrate was washed with brine (100 mL*2). The organic phase was dried over Na₂SO₄ and concentrated to give a residue. The residue was purified by column chromatography. Compound 2H-2 (48.0 g, 205 mmol, 98.0% yield) was obtained as colorless oil. ¹H NMR: (400 MHz, CDCl₃) δ 4.43 (s, 2H), 3.67 (s, 3H), 3.18 (s, 3H), 0.92 (s, 9H), 0.12 (s, 6H)

Step 3: To a solution of compound 2H-1 (10.0 g, 72.9 mmol, 1.00 eq) in THF (200 mL) was added dropwise LDA (2.00 M, 80.2 mL, 2.20 eq) at −78° C. The mixture was stirred at −78° C. for 0.5 hr and −20° C. for 0.5 hr, then cooled to −78° C. A solution of compound 2H-2 (18.7 g, 80.2 mmol, 1.10 eq) in THF (20.0 mL) was added dropwise at −78° C., the mixture was stirred at −78° C. for 0.5 hr and 25° C. for 2 hrs. The mixture was poured into a solution of ice water (100 mL). A solution of HCl (1.00 M) was added to adjust pH to about 6. The aqueous layer was extracted with ethyl acetate (300 mL*3). The combined organic phase was dried over Na₂SO₄ and concentrated to give a residue. Compound 2H-3 (10.0 g, 32.3 mmol, 44.3% yield) was obtained as colorless oil. LC-MS: (M−H)⁻: 307.8. ¹H NMR: (400 MHz, CDCl₃) δ 9.27 (s, 1H), 8.73 (d, J=5.2 Hz, 1H), 7.24 (d, J=5.2 Hz, 1H), 4.16-3.91 (m, 4H), 0.94 (s, 9H), 0.15 (s, 6H)

Step 4: A mixture of compound 2H-3 (10.0 g, 32.3 mmol, 1.00 eq) and NH₄OAc (19.9 g 258 mmol, 8.00 eq) was stirred at 130° C. for 0.5 hr. H₂O (200 mL) was added to the mixture, the mixture was stirred at 25° C. for 0.5 hr, then filtered. The filtrate cake was concentrated to give a residue. Compound 2H-4 (2.20 g, 12.4 mmol, 38.6% yield) was obtained as brown solid. LC-MS: (M+H)⁺: 177.2. ¹H NMR: (400 MHz, DMSO-d₆) δ 9.24 (s, 1H), 8.64 (d, J=5.2 Hz, 1H), 7.55 (d, J=5.6 Hz, 1H), 6.53 (s, 1H), 4.36 (s, 2H)

Step 5: To a solution of compound 2H-4 (2.00 g, 11.3 mmol, 1.00 eq) in DMF (8.00 mL) and DCM (5.00 mL) was added TBSCl (5.13 g, 34.0 mmol, 4.19 mL, 3.00 eq) and imidazole (3.09 g, 45.4 mmol, 4.00 eq), then the mixture was stirred at 25° C. for 0.5 hr. The mixture was diluted with H₂O (20.0 mL) and extracted with DCM (20.0 ml.*3). The combined organic phase was washed with brine (100 ml*3), dried over Na₂SO₄ and concentrated to give a residue. The residue was purified by column chromatography (SiO₂, Petroleum ether/Ethyl acetate=100/1 to 0/1). Compound 2H-5 (3.00 g, 10.3 mmol, 90.9% yield) was obtained as yellow solid. LC-MS: (M+H)⁺: 291.1. ¹H NMR: (400 MHz, CDCl₃). δ 11.55 (s, 1H), 9.25 (s, 1H), 8.65 (d, J=5.6 Hz, 1H), 7.58 (d, J=5.6 Hz, 1H), 6.51 (s, 1H), 4.56 (s, 2H), 0.92 (s, 9H), 0.12 (s, 6H)

Step 6: To a solution of compound 2H-5 (2.00 g, 6.89 mmol, 1.00 eq) in Py (30.0 mL) was added Tf₂O (3.89 g, 13.7 mmol, 2.27 mL, 2.00 eq) at 0° C., then the mixture was stirred at 25° C. for 2 hrs. The reaction mixture was concentrated under reduced pressure to remove Py. The residue was diluted with H₂O 50.0 mL and extracted with ethyl acetate (50.0 mL*3). The combined organic layers were washed with brine (120 mL*2), concentrated under reduced pressure to give a residue. The residue was purified by column chromatography. Compound 2H-6 (600 mg, 1.42 mmol, 20.6% yield) was obtained as yellow solid. LC-MS: (M+H)⁺: 423.2

Step 7: A mixture of compound 2H-6 (500 mg, 1.18 mmol, 1.00 eq), compound 2H-7 (751 mg, 1.18 mmol, 1.00 eq), K₃PO₄ (753 mg, 3.55 mmol, 3.00 eq), Pd(dtbpf)Cl₂, (77.1 mg, 118 μmol, 0.100 eq) in dioxane (6.00 mL), H₂O (1.50 mL) was degassed and purged with N₂ for 3 times. The mixture was stirred at 25° C. for 1 hr under N₂ atmosphere. The reaction mixture was partitioned between H₂O 20.0 mL and ethyl acetate (20.0 ml*3). The organic phase was separated, washed with brine (60.0 mL*2), concentrated under reduced pressure to give a residue. The residue was purified by Prep-TLC (SiO₂, Petroleum ether/Ethyl acetate=2/1). Compound 2H-8 (700 mg, crude) was obtained as yellow solid. LC-MS: (M+H)⁺: 763.4

Step 8: To a solution of compound 2H-8 (700 mg, 917 μmol, 1.00 eq) in THF (5.00 mL) was added TBAF (1.00 M, 1.84 mL, 2.00 eq) at 0° C. The mixture was stirred at 25° C. for 0.5 hr. The reaction mixture was partitioned between H₂O 15.0 mL and ethyl acetate (15.0 ml*3). The organic phase was separated, washed with brine (20.0 mL*2), concentrated under reduced pressure to give a residue. The residue was purified by Prep-TLC (SiO₂, Petroleum ether/Ethyl acetate=1/1). Compound 2H-9 (310 mg, 477 μmol, 52.0% yield) was obtained as yellow solid. LC-MS: (M+H)⁺: 649.3. ¹H NMR: (400 MHz, CDCl₃) δ 8.95 (d, J=10.4 Hz, 1H), 8.71 (d, J=6.0 Hz, 1H), 8.38-8.35 (m, 1H), 7.81 (s, 1H), 7.76 (t, J=7.2 Hz, 2H), 7.63 (d, J=7.2 Hz, 1H), 7.36-7.29 (m, 3H), 6.45-6.41 (m, 1H), 5.11 (d, J=6.8 Hz, 1H), 5.05 (s, 2H), 3.75-3.62 (m, 5H), 2.95-2.77 (m, 4H), 2.43-2.16 (m, 2H), 2.14-2.07 (m, 2H), 2.03-1.88 (m, 2H)

Step 9: A solution of compound 2H-9 (300 mg, 462 μmol, 1.00 eq) in DMSO (5.00 mL) was added IBX (259 mg, 925 μmol, 2.00 eq) at 25° C. and stirred for 0.5 hr. The reaction mixture was quenched by addition sat. Na₂SO₃ aq. 5.00 mL at 25° C. and stirred for 0.2 hr, and then extracted with ethyl acetate (5.00 mL*3). The combined organic layers were washed with brine (10.0 mL*2), concentrated under reduced pressure to give a residue. The residue was purified by Prep-TLC (SiO₂, Petroleum ether/Ethyl acetate=1/1). Compound 2H-10 (150 mg, 231 μmol, 50.1% yield) was obtained as a white solid. LC-MS: (M+H)⁺: 647.3

Step 10: To a solution of compound 2H-10 (120 mg, 185 μmol, 1.00 eq) in THF (2.00 mL) was added compound 2H-11 (79.0 mg, 927 μmol, 91.6 μL, 5.00 eq) and AcOH (1.11 mg, 18.5 μmol, 1.06 μL, 0.100 eq) stirred at 25° C. for 0.2 hr, then was added NaBH₃CN (14.0 mg, 222 μmol, 1.20 eq) and the mixture was stirred at 25° C. for 0.2 hr. The reaction mixture was partitioned between H₂O 5.00 mL and ethyl acetate (5.00 mL*3). The organic phase was separated, washed with brine (10.0 mL*1), concentrated under reduced pressure to give a residue. The residue was purified by Prep-TLC (SiO₂, Dichloromethane/Methanol=10/1). Compound 2H-12 (130 mg, 181 μmol, 97.8% yield) was obtained as a white solid. LC-MS: (M+H)⁺: 716.6. ¹H NMR: (400 MHz, DMSO-d₆) δ 8.77-8.64 (m, 2H), 8.62-8.56 (d, J=7.2 Hz, 1H), 8.34 (t, J=8.8 Hz, 1H), 8.01 (d, J=6.0 Hz, 1H), 7.98 (s, 1H), 7.82-7.75 (m, 1H), 7.66 (d, J=7.2 Hz, 1H), 7.46-7.37 (m, 1H), 7.31-7.29 (m, 1H), 7.19-7.15 (m, 1H), 4.95-4.76 (m, 1H), 3.85 (d, J=3.2 Hz, 1H), 3.82-3.77 (m, 2H), 3.71 (d, J=4.8 Hz, 3H), 3.64-3.54 (m, 4H), 3.30 (s, 2H), 3.06-2.82 (m, 2H), 2.24 (d, J=13.2 Hz, 2H), 1.79-1.73 (m, 3H), 1.66-1.51 (m, 6H), 1.46-1.42 (m, 2H)

Step 11: A solution of compound 2H-12 (120 mg, 167 μmol, 1.00 eq) in HCl (6.00 M, 0.500 mL, 17.8 eq) was stirred at 60° C. for 1 hr. The reaction mixture was concentrated under reduced pressure to give a residue. The residue was purified by Prep-HPLC (column: Welch Ultimate C 18 150*25 mm*5 um; mobile phase: [water (HCl)-ACN]; gradient: 20%-50% B over 10 min). Compound 994 (100.45 mg, 135 μmol, 80.6% yield, 99.3% purity, HCl) was obtained as a yellow solid. LC-MS: (M+H)⁺: 702.4. ¹H NMR: (400 MHz, DMSO-d₆) δ 8.94 (d, J=10.8 Hz, 1H), 8.82 (t, J=5.6 Hz, 1H), 8.61 (t, J=9.2 Hz, 1H), 8.38 (dd, J₁=8.4 Hz, J₂=2.8 Hz, 1H), 8.26 (s, 1H), 8.19 (t, J=6.0 Hz, 1H), 7.87-7.80 (m, 1H), 7.74 (d, J=7.2 Hz, 1H), 7.48-7.41 (m, 1H), 7.32-7.25 (m, 2H), 4.81-4.78 (m, 1H), 4.66 (s, 2H), 3.94-3.79 (m, 1H), 3.56-3.41 (m, 4H), 3.38-3.19 (m, 2H), 3.10-2.88 (m, 4H), 2.41-2.27 (m, 2H), 2.26-2.13 (m, 1H), 1.87-1.59 (m, 7H), 1.39-1.37 (m, 1H)

### Example 49 General Scheme for Common Intermediate 3

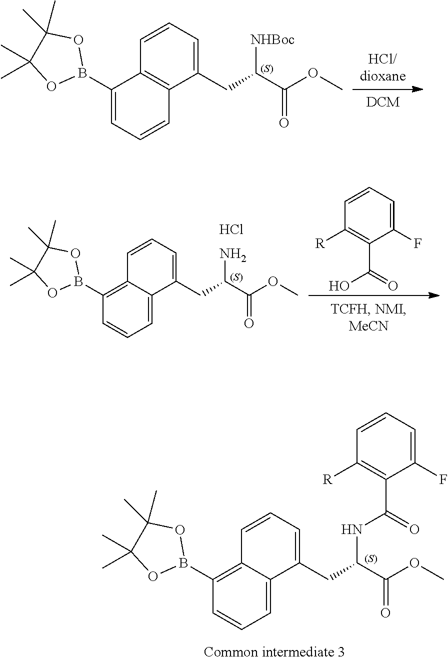

In some embodiments, R of common intermediate 3, is an R group as exemplified in Examples 50 to 55. In some embodiments, R of common intermediate 2 is an R groups as exemplified in compounds 995 to 1039.

### Example 50: Synthesis of Intermediate 3 (Int3)

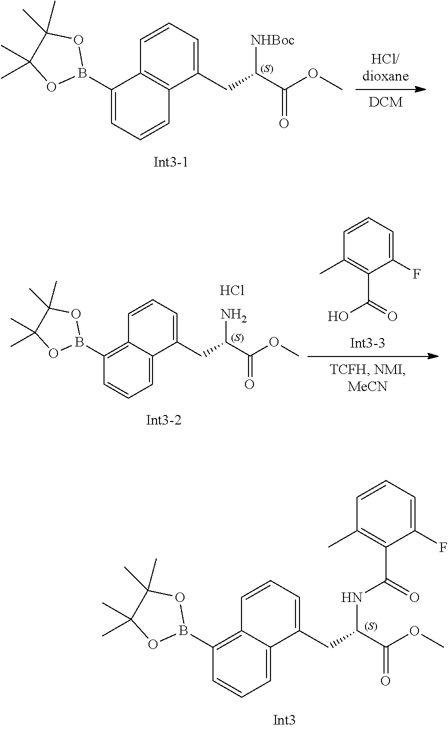

Step 1: To a solution of compound Int3-1 (5.00 g, 11.0 mmol, 1.00 eq) and in DCM (50.0 mL) was added HCl/dioxane (4.00 M, 10.0 mL, 3.64 eq), the mixture was stirred at 20° C. for 2.5 hrs. The reaction mixture was concentrated under reduced pressure to give a residue. Compound Int3-2 (4.30 g, crude, HCl) was obtained as yellow solid. LC-MS: (M+H)⁺: 355.8

Step 2: To a solution of compound Int3-2 (508 mg, 1.30 mmol, 1.00 eq, HCl) and compound Int3-3 (200 mg, 1.30 mmol, 1.00 eq) in ACN (10.0 mL) was added TCFH (1.09 g, 3.89 mmol, 3.00 eq) and NMI (639 mg, 7.79 mmol, 621 μL, 6.00 eq), the mixture was stirred at 20° C. for 1.5 hrs. The reaction mixture was concentrated under reduced pressure to remove ACN, diluted with water 30.0 mL, and extracted with dichloromethane 90.0 mL (30.0 mL*3). The combined organic layers were washed with brine 30.0 mL (30.0 mL*1), dried over Na₂SO₄, filtered and concentrated under reduced pressure to give a residue. The residue was purified by column chromatography (SiO₂, Petroleum ether:Ethyl acetate=20:1 to 8:1). Compound Int3 (573 mg, 1.15 mmol, 89.0% yield, 99.0% purity) was obtained as white solid. LC-MS: (M+H)⁺: 492.2. ¹H NMR: (400 MHz, CDCl₃) δ 8.72 (d, J=8.4 Hz, 1H), 8.32 (d, J=8.4 Hz, 1H), 8.12-8.05 (m, 1H), 7.60-7.52 (m, 1H), 7.47-7.41 (m, 1H), 7.33 (d, J=6.8 Hz, 1H), 7.25-7.20 (m, 1H), 6.99-6.85 (m, 2H), 6.34 (d, J=8.0 Hz, 1H), 5.28-5.17 (m, 1H), 3.76-3.64 (m, 2H), 3.63 (s, 3H), 2.32 (s, 3H), 1.43 (s, 9H).

### Example 51: General Scheme 3A

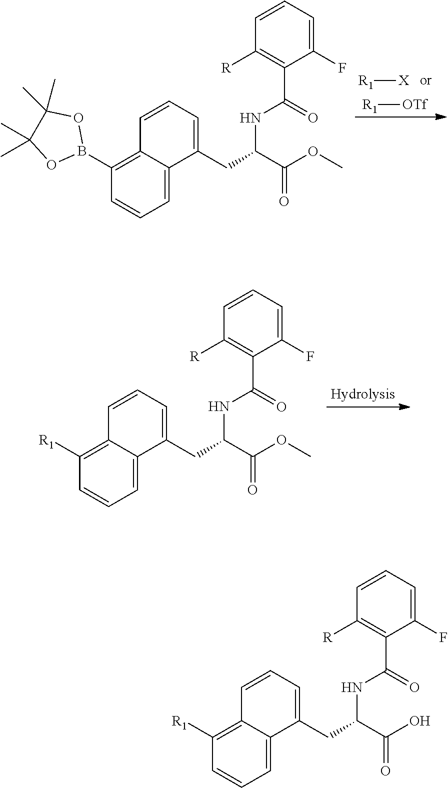

The proceeding compounds were synthesized according to the procedures described in General Scheme 3A using Common Intermediate 3 or Int3 as an intermediate.

### Synthetic Scheme of Compound 995

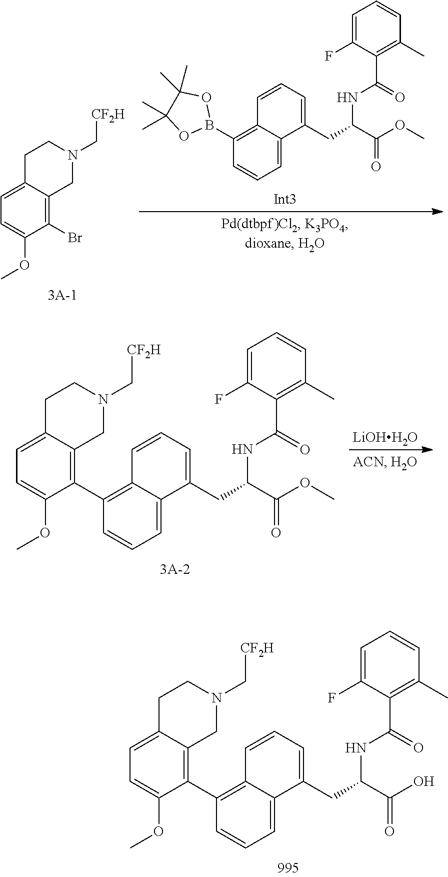

Step 1: To a solution of compound 3A-1 (39.0 mg, 127 μmol, 1.00 eq), compound Int3 (62.3 mg, 127 μmol, 1.00 eq), Pd(dtbpf)Cl₂ (8.26 mg, 12.7 μmol, 0.100 eq) and K₃PO₄ (53.8 mg, 254 μmol, 2.00 eq) in dioxane (2.00 mL) and H₂O (0.500 mL) was degassed and purged with N₂ for 3 times, and then the mixture was stirred at 25° C. for 1 hr. The reaction mixture was diluted with extracted with ethyl acetate 90.0 mL (30.0 mL*3). The combined organic layers were washed with water 30.0 mL (30.0 mL*1), dried over Na₂SO₄, filtered, and concentrated under reduced pressure to give a residue. The residue was purified by Prep-TLC (SiO₂, Petroleum ether:Ethyl acetate=3:1). Compound 3A-2 (51.0 mg, 86.4 μmol, 68.1% yield, 100% purity) was obtained as yellow solid. LC-MS: (M+H)⁺: 591.4.

Step 2: To a solution of compound 3A-2 (41.0 mg, 69.4 μmol, 1.00 eq) in ACN (2.00 mL) was added a solution of LiOH·H₂O (3.50 mg, 83.30 μmol, 1.20 eq) in H₂O (0.500 mL), the mixture was stirred at 25° C. for 0.5 hr. The reaction mixture was concentrated under reduced pressure to give residue. The residue was purified by Prep-HPLC (column: Waters xbridge 150*25 mm 10 um; mobile phase: [water (NH₄HCO₃)-ACN]; gradient: 25%-45% B over 14 min). Compound 995 (37.27 mg, 64.5 μmol, 92.9% yield, 99.8% purity) was obtained as yellow solid. LC-MS: (M+H)⁺: 577.2. ¹H NMR: (400 MHz, DMSO-d₆) δ 8.53 (dd, J₁=33.6 Hz, J₂=8.4 Hz, 1H), 8.28 (dd, J₁=8.4 Hz, J₂=2.4 Hz, 1H), 7.66-7.57 (m, 1H), 7.49-7.40 (m, 1H), 7.31-7.19 (m, 4H), 7.13 (dd, J₁=8.4 Hz, J₂=3.2 Hz, 1H), 7.03-6.94 (m, 3H), 6.09-5.74 (m, 1H), 4.75-4.61 (m, 1H), 3.82-3.73 (m, 1H), 3.53 (d, J=8.4 Hz, 3H), 3.34-3.25 (m, 2H), 2.92-2.72 (m, 5H), 2.65-2.53 (m, 2H), 2.03 (d, J=21.2 Hz, 3H)

### Synthesis of Compound 996

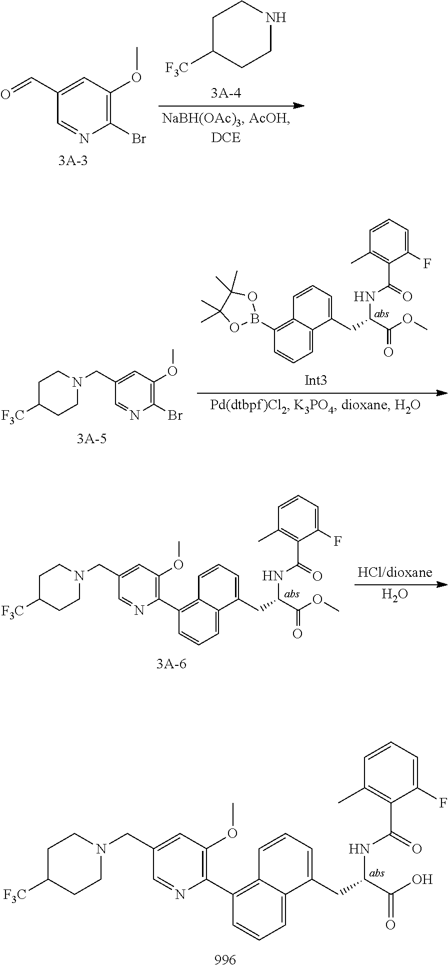

Step 1: To a solution of compound 3A-3 (100 mg, 462 μmol, 1.00 eq) was added compound 3A-4 (212 mg, 1.39 mmol, 3.00 eq), CH₃COOH (55.6 mg, 925 μmol, 53.0 μL, 2.00 eq) in DCE (1.00 mL), the mixture was stirred at 25° C. for 1 hr. Then NaBH(OAc)₃ (147 mg, 694 μmol, 1.50 eq) was added and the mixture was stirred at 25° C. for 1 hr. The reaction mixture was diluted with H₂O 20.0 mL and extracted with ethyl acetate 15.0 mL (5.00 mL*3). The combined organic layers were concentrated under reduced pressure to give a residue. The residue was purified by Prep-TLC (SiO₂, Petroleum ether/Ethyl acetate=1/1; R_{f}=0.38). Compound 3A-5 (90.0 mg, 254 μmol, 55.0% yield) was obtained as a yellow oil. LC-MS: (M+H)⁺: 353.0. ¹H NMR: (400 MHz, DMSO-d₆). δ 7.88 (brs, 1H), 7.27 (s, 1H), 4.02-3.90 (m, 2H), 3.50 (s, 3H), 2.97-2.95 (m, 1H), 2.06-1.98 (m, 4H), 1.86 (d, J=12.0 Hz, 2H), 1.69-1.62 (m, 2H).

Step 2: A mixture of compound 3A-5 (146 mg, 297 μmol, 1.50 eq), compound Int3 (70.0 mg, 198 μmol, 1.00 eq), K₃PO₄ (84.1 mg, 396 μmol, 2.00 eq), Pd(dtbpf)Cl₂ (25.8 mg, 39.6 μmol, 0.200 eq) in dioxane (2.00 mL) and H₂O (0.500 mL) was degassed and purged with N₂ for 3 times, and then the mixture was stirred at 25° C. for 4 hrs under N₂ atmosphere. The reaction mixture was diluted with H₂O 20.0 mL and extracted with ethyl acetate 15.0 mL (5.00 mL*3). The combined organic layers concentrated under reduced pressure to give a residue. The residue was purified by Prep-TLC (SiO₂, Dichloromethane:Methanol=30/1; R_{f}=0.39). Compound 3A-6 (80.0 mg, 125 μmol, 63.3% yield) was obtained as a yellow oil. LC-MS: (M+H)⁺: 638.1.

Step 3: To a solution of compound 3A-6 (75.0 mg, 117 μmol, 1.00 eq) and H₂O (1.00 mL) was added HCl/dioxane (4.00 M, 2.14 mL, 72.8 eq). The mixture was stirred at 60° C. for 2 hrs. The reaction mixture was concentrated under reduced pressure to give a residue. The residue was purified by Prep-HPLC (column: Phenomenex luna C18 150*25 mm*10 um; mobile phase: [water (FA)-ACN]; gradient: 13%-43% B over 10 min). Compound 996 (69.70 mg, 111 μmol, 94.4% yield, 99.4% purity) was obtained as a white solid. LC-MS: (M+H)⁺: 624.4. ¹H NMR: (400 MHz, DMSO-d₆) δ 9.02 (d, J=8.0 Hz, 1H), 8.22 (br d, J=9.2 Hz, 2H), 7.74-7.63 (m, 1H), 7.55 (brs, 1H), 7.49-7.43 (m, 2H), 7.37-7.24 (m, 3H), 7.08-6.95 (m, 2H), 4.87-4.74 (m, 1H), 3.85-3.61 (m, 6H), 3.43-3.33 (m, 1H), 3.04 (br s, 2H), 2.41-2.30 (m, 1H), 2.16-2.05 (m, 5H), 1.94-1.77 (m, 2H), 1.66-1.46 (m, 2H).

The compounds set forth in Table 24, were prepared using the synthetic procedures of Example 51, or analogous procedures as provided herein.

TABLE 24
Structure and Spectroscopic data for Compounds 740 and 997 to 1008

| No. | Structure |
|---|---|
| 740 | 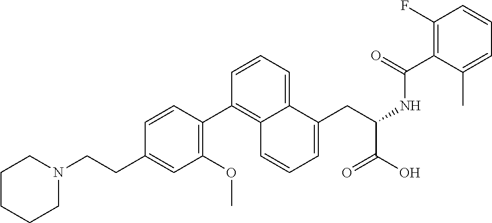 [SMILES: COc1cc(CCN2CCCCC2)ccc1-c1cccc2c(C[C@H](NC(=O)c3c(C)cccc3F)C(=O)O)cccc12] |
| | m/z = 569.3 |
| 997 | 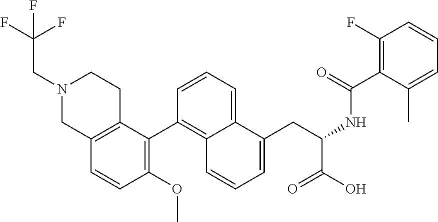 [SMILES: COc1ccc2c(c1-c1cccc3c(C[C@H](NC(=O)c4c(C)cccc4F)C(=O)O)cccc13)CCN(CC(F)(F)F)C2] |
| | m/z = 595.1 |
| 998 | 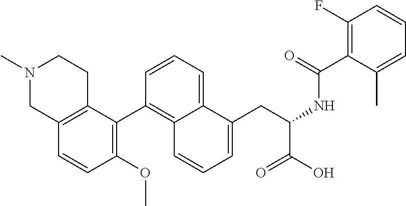 [SMILES: COc1ccc2c(c1-c1cccc3c(C[C@H](NC(=O)c4c(C)cccc4F)C(=O)O)cccc13)CCN(C)C2] |
| | m/z = 527.2 |
| 999 | 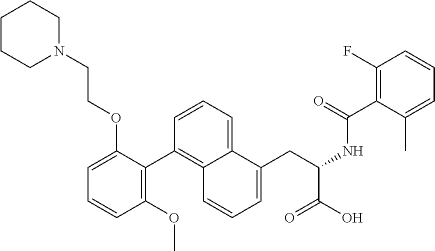 [SMILES: COc1cccc(OCCN2CCCCC2)c1-c1cccc2c(C[C@H](NC(=O)c3c(C)cccc3F)C(=O)O)cccc12] |
| | m/z = 585.3 |
| 1000 | 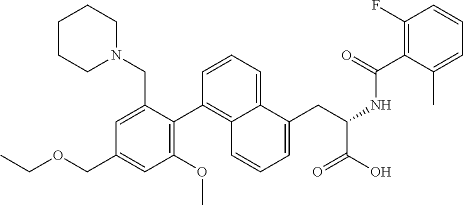 [SMILES: CCOCc1cc(CN2CCCCC2)c(-c2cccc3c(C[C@H](NC(=O)c4c(C)cccc4F)C(=O)O)cccc23)c(OC)c1] |
| | m/z = 613.4 |
| 1001 | 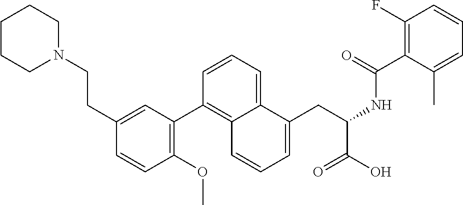 [SMILES: COc1ccc(CCN2CCCCC2)cc1-c1cccc2c(C[C@H](NC(=O)c3c(C)cccc3F)C(=O)O)cccc12] |
| | m/z = 569.3 |
| 1002 | 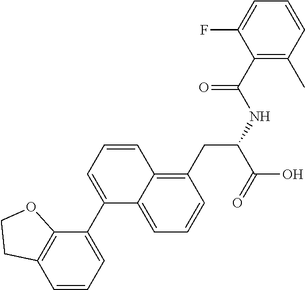 [SMILES: Cc1cccc(F)c1C(=O)N[C@@H](Cc1cccc2c(-c3cccc4c3OCC4)cccc12)C(=O)O] |

m/z = 470.2

| | |
|---|---|
| 1003 | 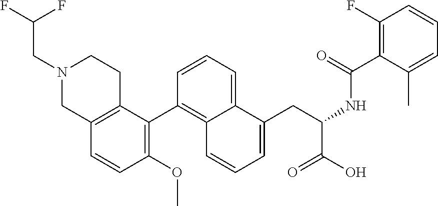 [SMILES: COc1ccc2c(c1-c1cccc3c(C[C@H](NC(=O)c4c(C)cccc4F)C(=O)O)cccc13)CCN(CC(F)F)C2] |
| | m/z = 577.1 |
| 1004 | 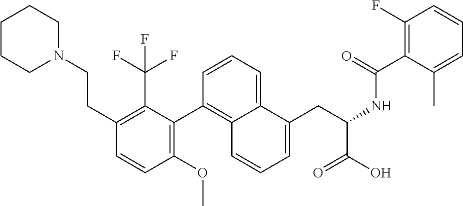 [SMILES: COc1ccc(CCN2CCCCC2)c(C(F)(F)F)c1-c1cccc2c(C[C@H](NC(=O)c3c(C)cccc3F)C(=O)O)cccc12] |
| | m/z = 637.2 |
| 1005 | 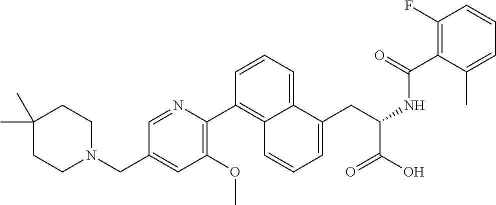 [SMILES: COc1cc(CN2CCC(C)(C)CC2)cnc1-c1cccc2c(C[C@H](NC(=O)c3c(C)cccc3F)C(=O)O)cccc12] |
| | m/z = 584.3 |
| 1006 | 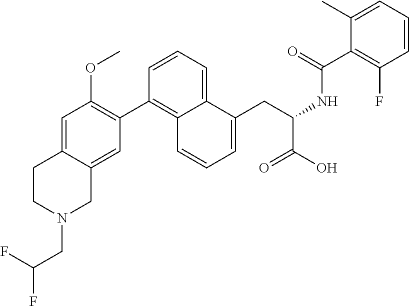 [SMILES: COc1cc2c(cc1-c1cccc3c(C[C@H](NC(=O)c4c(C)cccc4F)C(=O)O)cccc13)CN(CC(F)F)CC2] |
| | m/z = 577.2 |
| 1007 | 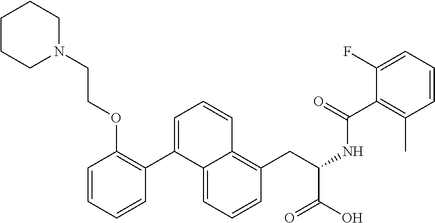 [SMILES: Cc1cccc(F)c1C(=O)N[C@@H](Cc1cccc2c(-c3ccccc3OCCN3CCCCC3)cccc12)C(=O)O] |
| | m/z = 555.3 |
| 1008 | 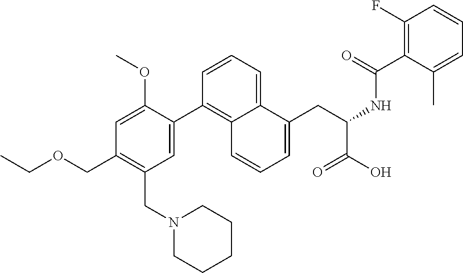 [SMILES: CCOCc1cc(OC)c(-c2cccc3c(C[C@H](NC(=O)c4c(C)cccc4F)C(=O)O)cccc23)cc1CN1CCCCC1] |
| | m/z = 613.4 |

### Example 52: General Scheme 3B

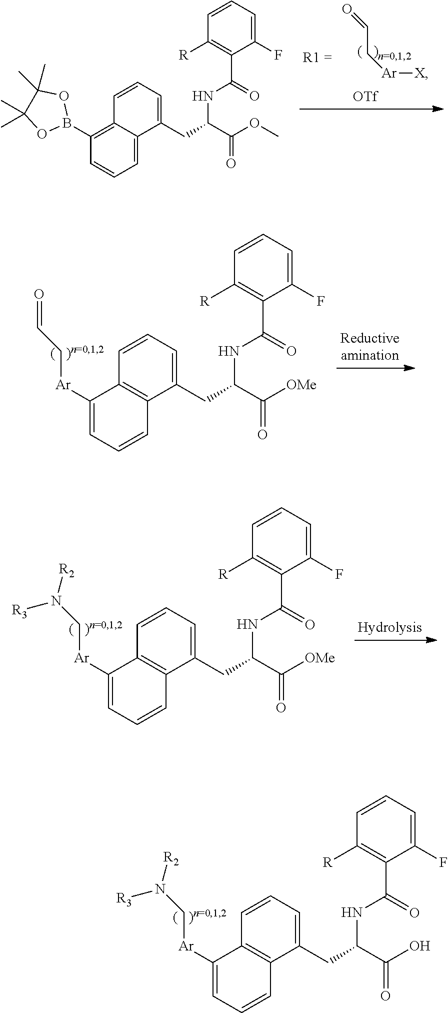

The proceeding compounds were synthesized according to the procedures described in General Scheme 3B using Common Intermediate 3 or Int3 as an intermediate.

### Synthesis of Compound 1009

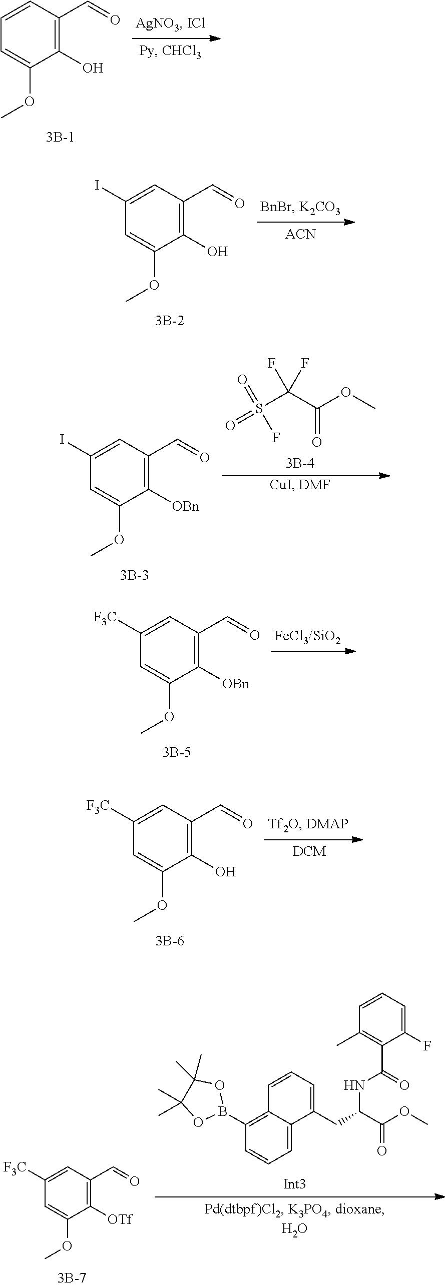

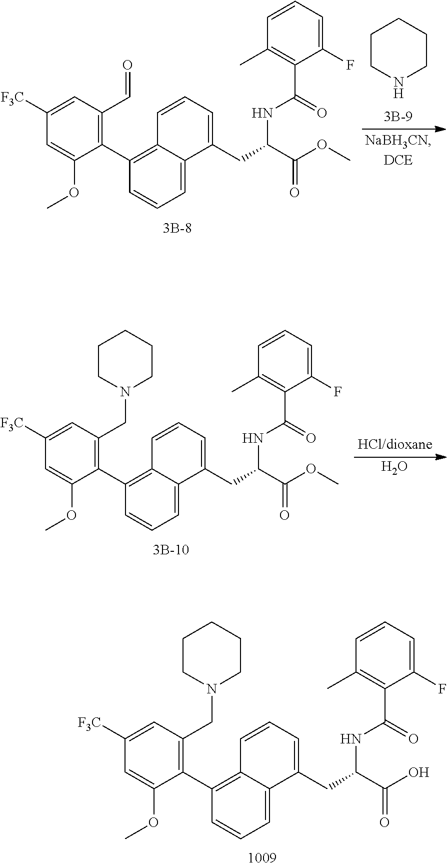

Step 1: To a solution of AgNO₃ (6.70 g, 39.4 mmol, 1.00 eq) in Py (20.0 mL) and CHCl₃ (50.0 mL), then ICI (6.40 g, 39.4 mmol, 2.01 mL, 1.00 eq) in CHCl₃ (10.0 mL) was added dropwise over a period of 15 mins with constant stirring at 25° C. The above mixture was continue stirred for another 15 mins, and then compound 3B-1 (6.00 g, 39.4 mmol, 1.00 eq) was dissolved in CHCl₃ (10.0 mL) dropwise to above mixture with stirring. The mixture was stirred at 25° C. for 3 hrs. The reaction mixture was diluted with petroleum ether (50.0 mL). The mixture was filtered. The resulting residue was washed with a mixture of CHCl₃/Petroleum ether (100 mL), concentrated under reduced pressure. the mixture was redissolved in CHCl₃ (50.0 mL), and washed successively with 5.00% HCl (50.0 mL), 5.00% Na₂SO₃ (50.0 mL), and H₂O (50.0 mL). The combined organic layers were washed with brine 60.0 mL (30.0 mL*2), dried over Na₂SO₄, filtered, and concentrated under reduced pressure to give a residue. The residue was purified by column chromatography (Petroleum ether:Ethyl acetate=5:1, R_{f}=0.49, SiO₂, Petroleum ether:Ethyl acetate=1:0 to 8:1). Compound 3B-2 (8.00 g, 28.6 mmol, 72.4% yield, 99.3% purity) was obtained as a light yellow solid. LC-MS: (M−H)⁺: 276.6. ¹H NMR: (400 MHz, DMSO-d₆) δ 10.41 (s, 1H), 10.17 (s, 1H), 7.47 (dd, J₁=20.0 Hz, J₂=1.6 Hz, 2H), 3.86 (s, 3H).

Step 2: To a solution of compound 3B-2 (7.00 g, 25.0 mmol, 1.00 eq) in ACN (70.0 mL) was added BnBr (8.55 g, 50.0 mmol, 5.94 mL, 2.00 eq) and K₂CO₃ (6.91 g, 50.0 mmol, 2.00 eq). The mixture was stirred at 50° C. for 2 hrs. The residue was diluted with H₂O 120 mL and extracted with ethyl acetate 210 mL (70.0 mL*3). The combined organic layers were washed with brine 40.0 mL (20.0 mL*2), dried over Na₂SO₄, filtered, and concentrated under reduced pressure to give a residue. The residue was purified by column chromatography (Petroleum ether:Ethyl acetate=5:1, R_{f}=0.54, SiO₂, Petroleum ether:Ethyl acetate=1:1 to 40:1). Compound 3B-3 (9.00 g, 22.7 mmol, 90.7% yield, 92.8% purity) was obtained as a white solid. ¹H NMR. (400 MHz, DMSO-d₆) δ 9.96 (s, 1H), 7.65 (d, J=2.0 Hz, 1H), 7.47 (d, J=2.0 Hz, 1H), 7.42-7.32 (m, 5H), 5.15 (s, 2H), 3.94 (s, 3H).

Step 3: To a solution of compound 3B-3 (1.50 g, 3.78 mmol, 1.00 eq) and compound 3B-4 (3.63 g, 18.9 mmol, 2.41 mL, 5.00 eq) in DMF (15.0 mL) was added CuI (2.16 g, 11.3 mmol, 3.00 eq). The mixture was stirred at 120° C. for 12 hrs. The residue was diluted with H₂O 100 mL and extracted with ethyl acetate 90.0 mL (30.0 mL*3). The combined organic layers were washed with brine 20.0 mL (10.0 mL*2), dried over Na₂SO₄, filtered, and concentrated under reduced pressure to give a residue. The residue was purified by column chromatography (Petroleum ether:Ethyl acetate=5:1, R_{f}=0.46, SiO₂, Petroleum ether:Ethyl acetate=1:0 to 5:1). Compound 3B-5 (950 mg, 2.48 mmol, 65.6% yield, 81.0% purity) was obtained as colorless oil. ¹H NMR: (400 MHz, DMSO-d₆) δ 10.11 (s, 1H), 7.68 (d, J=2.0 Hz, 1H), 7.48 (d, J=1.6 Hz, 1H), 7.45-7.35 (m, 5H), 5.27 (s, 2H), 4.03 (s, 3H).

Step 4: To a solution of FeCl₃ (186 mg, 1.15 mmol, 66.5 μL, 1.10 eq) in MeOH (2.00 mL) and THF (4.00 mL) was added SiO₂ (4.05 g, 67.4 mmol, 64.6 eq). The mixture was concentrated under reduced pressure (water pump) on the rotary evaporator and remained spinning for 1 hr at 30° C. Then the mixture was transferred to a vacuum (oil pump) at 30° C. for 1 hr to get a yellow solid. To a solution of compound 3B-5 (400 mg, 1.04 mmol, 1.00 eq) in DCM (4.00 mL) was added FeCl₃/SiO₂ (the same as above) at 25° C. An immediate deep red color formed on the surface of the reagent. The mixture was removed under reduced pressure on the rotary evaporator and remained spinning for 2 hrs at 50° C. The reaction mixture was quenched by H₂O (30.0 mL) at 25° C. and diluted with ethyl acetate (90.0 mL). Compound 3B-6 (280 mg, crude) was obtained as colorless oil. LC-MS: (M−H)⁻: 218.9.

Step 5: To a solution of compound 3B-6 (480 mg, 2.18 mmol, 1.00 eq) in DCM (2.00 mL) was added DMAP (959 mg, 7.85 mmol, 3.60 eq) and Tf₂O (1.85 g, 6.54 mmol, 1.08 mL, 3.00 eq) at 0° C. The mixture was stirred at 0° C. for 1 hr. The residue was diluted with H₂O 30.0 mL and extracted with DCM 90.0 mL (30.0 mL*3). The combined organic layers were washed with brine 20.0 mL (20.0 mL*1), dried over Na₂SO₄, filtered, and concentrated under reduced pressure to give a residue. The residue was purified by Prep-TLC (SiO₂, Petroleum ether:Ethyl acetate=5:1, R_{f}=0.41). Compound 3B-7 (365 mg, 1.04 mmol, 47.5% yield) was obtained as a white solid. ¹H NMR: (400 MHz, DMSO-d₆) δ 10.09 (s, 1H), 8.01 (d, J=2.4 Hz, 2H), 4.06 (s, 3H).

Step 6: A mixture of compound 3B-7 (100 mg, 284 μmol, 1.00 eq), compound Int3 (167 mg 341 μmol, 1.20 eq), Pd(dtbpf)Cl₂ (18.5 mg, 28.4 μmol, 0.100 eq), K₃PO₄ (181 mg, 852 μmol, 3.00 eq) in dioxane (2.00 mL) and H₂O (0.500 mL) was degassed and purged with N₂ for 3 times, and then the mixture was stirred at 25° C. for 2 hrs under N₂ atmosphere. The reaction mixture was concentrated under reduced pressure to remove dioxane. The residue was diluted with H₂O 20.0 mL and extracted with ethyl acetate 60.0 mL (20.0 mL*3). The combined organic layers were washed with brine 10.0 ml. (10.0 ml.*1), dried over Na₂SO₁, filtered, and concentrated under reduced pressure to give a residue. The residue was purified by Prep-TLC (SiO₂, Petroleum ether:Ethyl acetate=1:1, R_{f}=0.43). Compound 3B-8 (110 mg, 177 μmol, 62.2% yield, 91.2% purity) was obtained as yellow oil. LC-MS: (M+H)⁺: 568.3.

Step 7: To a solution of compound 3B-8 (100 mg, 161 μmol, 1.00 eq) and compound 3B-9 (41.1 mg, 482 μmol, 47.6 μL, 3.00 eq) in DCE (2.00 mL) was stirred at 25° C. for 0.5 hr, then was added NaBH₃CN (15.2 mg, 241 μmol, 1.50 eq). The mixture was stirred at 25° C. for 2 hrs. The residue was diluted with H₂O 10.0 mL and extracted with DCM 60.0 mL (20.0 mL*3). The combined organic layers were washed with brine 10.0 mL (10.0 mL*1), dried over Na₂SO₄, filtered, and concentrated under reduced pressure to give a residue. The residue was purified by Prep-TLC (SiO₂, DCM:MeOH=10:1, R_{f}=0.50). Compound 3B-10 (70.0 mg, 103 μmol, 64.1% yield, 93.7% purity) was obtained as yellow oil. LC-MS: (M+H)⁺: 637.2.

Step 8: To a solution of compound 3B-10 (60.0 mg, 88.3 μmol, 1.00 eq) in H₂O (0.500 mL) was added HCl/dioxane (4.00 M, 1.00 mL, 45.3 eq). The mixture was stirred at 60° C. for 2 hrs. The reaction mixture was concentrated under reduced pressure to give a residue. The residue was purified by Prep-HPLC (column: Waters xbridge 150*25 mm 10 um; mobile phase: [water (NH₄HCO₃)-ACN]; gradient: 33%-63% B over 14 min). Compound 1009 (32.42 mg, 51.4 μmol, 58.2% yield, 98.8% purity) was obtained as a white solid. LC-MS: (M+H)⁺: 623.5. ¹H NMR: (400 MHz, DMSO-d₆) δ8.97-8.81 (m, 1H), 8.23 (d, J=8.4 Hz, 1H), 7.66 (t, J=8.0 Hz, 1H), 7.58 (s, 1H), 7.46 (t, J=7.6 Hz, 1H), 7.34-7.23 (m, 4H), 7.07 (dd, J₁=8.0 Hz, J₂=2.0 Hz, 1H), 7.04-6.97 (m, 2H), 4.87-4.70 (m, 1H), 3.84-3.71 (m, 1H), 3.66 (d, J=7.2 Hz, 3H), 3.38-3.35 (m, 1H), 3.06-2.97 (m, 1H), 2.88 (t, J=13.2 Hz, 1H), 2.12-1.92 (m, 7H), 1.37-1.15 (m, 6H).

### Synthesis of Compound 1010

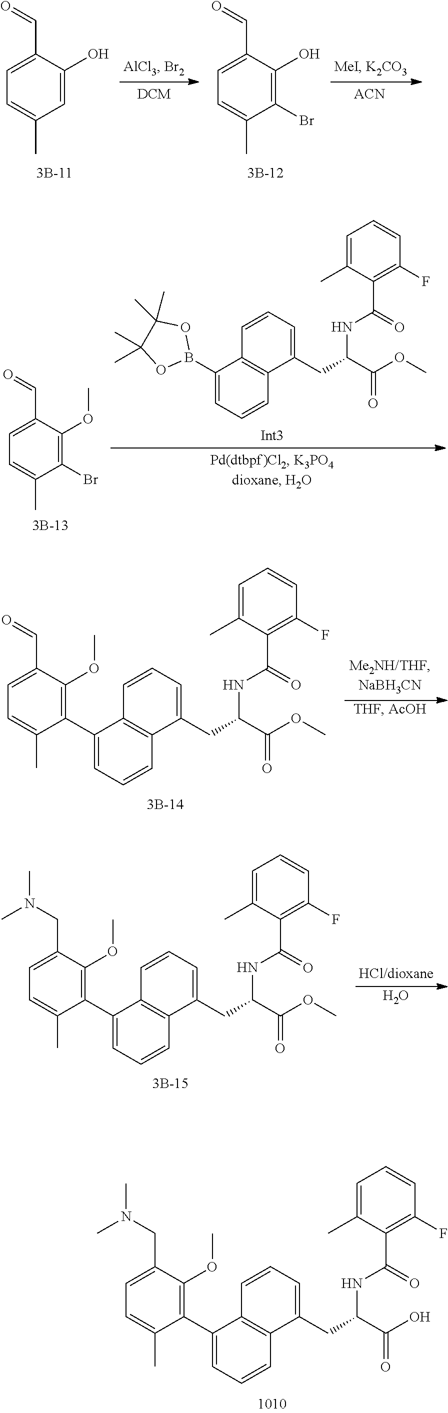

Step 1: To a solution of compound 3B-11 (1.00 g, 7.34 mmol, 1.00 eq) in DCM (15.0 mL) was added AlCl₃ (979 mg, 7.34 mmol, 401 μL, 1.00 eq) stirred at −20° C. for 30 min, then was added Br₂ (1.17 g, 7.34 mmol, 378 μL, 1.00 eq) at −20° C. The mixture was stirred at 25° C. for 6 hrs. The residue was diluted with H₂O 40.0 mL and extracted with DCM 120 mL (30.0 mL*4). The combined organic layers were washed with brine 20.0 mL (10.0 mL*2), dried over Na₂SO₄, filtered, and concentrated under reduced pressure to give a residue. The residue was purified by Prep-TLC (SiO₂, Petroleum ether:Ethyl acetate=10:1, R_{f}=0.37). Compound 3B-12 (159 mg, 739 μmol, 10.0% yield) was obtained as colorless oil. ¹H NMR: (400 MHz, DMSO-d₆) δ 11.42 (d, J=3.6 Hz, 1H), 9.99 (s, 1H), 7.68 (d, J=7.6 Hz, 1H), 7.09 (d, J=7.6 Hz, 1H), 2.43 (s, 3H).

Step 2: To a solution of compound 3B-12 (159 mg, 739 μmol, 1.00 eq) in ACN (3.00 mL) was added MeI (210 mg, 1.48 mmol, 92.1 μL, 2.00 eq) and K₂CO₃ (307 mg, 2.22 mmol, 3.00 eq). The mixture was stirred at 60° C. for 2 hrs. The residue was diluted with H₂O 20.0 mL and extracted with ethyl acetate 60.0 mL (20.0 mL*3). The combined organic layers were washed with brine 10.0 mL (10.0 mL*1), dried over Na₂SO₄, filtered, and concentrated under reduced pressure to give a residue. The residue was purified by Prep-TLC (SiO₂, Petroleum ether:Ethyl acetate=10:1, R_{f}=0.48). Compound 3B-13 (125 mg, 546 μmol, 73.8% yield) was obtained as a yellow solid. ¹H NMR: (400 MHz, DMSO-d₆) δ 10.20 (s, 1H), 7.67 (d, J=8.0 Hz, 1H), 7.33 (d, J=7.6 Hz, 1H), 3.93-3.90 (m, 3H), 2.45 (s, 3H).

Step 3: A mixture of compound 3B-13 (100 mg, 437 μmol, 1.00 eq), compound Int3 (232 mg, 437 μmol, 1.00 eq), Pd(dtbpf)Cl₂ (28.5 mg, 43.7 μmol, 0.100 eq), K₃PO₄ (278 mg, 1.31 mmol, 3.00 eq) in dioxane (2.00 mL) and H₂O (0.400 mL) was degassed and purged with N₂ for 3 times, and then the mixture was stirred at 25° C. for 2 hrs under N₂ atmosphere. The reaction mixture was concentrated under reduced pressure to remove dioxane. The residue was diluted with H₂O 20.0 mL and extracted with ethyl acetate 90.0 mL (30.0 mL*3). The combined organic layers were washed with brine 10.0 mL (10.0 mL*1), dried over Na₂SO₄, filtered, and concentrated under reduced pressure to give a residue. The residue was purified by Prep-TLC (SiO₂, Petroleum ether:Ethyl acetate=1:1, R_{f}=0.58). Compound 3B-14 (165 mg, 302 μmol, 69.2% yield, 94.1% purity) was obtained as yellow oil. LC-MS: (M+H)⁺: 514.2. ¹H NMR: (400 MHz, CDCl₃) δ 10.39 (s, 1H), 8.29 (d, J=8.8 Hz, 1H), 7.89 (d, J=8.0 Hz, 1H), 7.71-7.64 (m, 1H), 7.44-7.31 (m, 4H), 7.26-7.20 (m, 2H), 6.98 (d, J=7.6 Hz, 1H), 6.95-6.86 (m, 1H), 6.40 (d, J=7.6 Hz, 1H), 5.37-5.24 (m, 1H), 3.82-3.70 (m, 2H), 3.68 (d, J=3.6 Hz, 3H), 3.34 (d, J=4.0 Hz, 3H), 2.30 (d, J=2.8 Hz, 3H), 2.00, (s, 3H).

Step 4: To a solution of compound 3B-14 (145 mg, 266 μmol, 1.00 eq) in THF (2.00 ml) was added Me₂NH/THF (2.00 M, 1.00 mL, 7.53 eq) and AcOH (1.60 mg, 26.6 μmol, 1.52 μL, 0.100 eq) was stirred at 25° C. for 2 hrs, then was added NaBH₃CN (25.0 mg, 398 μmol, 1.50 eq). The residue was diluted with H₂O 20.0 mL and extracted with ethyl acetate 60.0 mL (20.0 mL*3). The combined organic layers were washed with brine 10.0 mL (10.0 mL*1), dried over Na₂SO₄, filtered and concentrated under reduced pressure to give a residue. Compound 3B-15 (168 mg, crude) was obtained as yellow oil. LC-MS: (M+H)⁺: 543.2. ¹H NMR: (400 MHz, CDCl₃) δ 8.23 (d, J=8.4 Hz, 1H), 7.69-7.60 (m, 1H), 7.46-7.29 (m, 5H), 7.26-7.20 (m, 1H), 7.12 (d, J=7.6 Hz, 1H), 6.98 (d, J=7.6 Hz, 1H), 6.94-6.86 (m, 1H), 6.39 (d, J=7.6 Hz, 1H), 5.33-5.24 (m, 1H), 3.73 (t, J=6.8 Hz, 2H), 3.66 (d, J=2.8 Hz, 3H), 3.17 (d, J=4.4 Hz, 3H), 2.65 (d, J=5.6 Hz, 5H), 2.43, (s, 6H), 2.29, (s, 3H), 1.90, (s, 3H).

Step 5: To a solution of compound 3B-15 (158 mg, 282 μmol, 1.00 eq) in H₂O (0.500 mL) was added HCl/dioxane (4.00 M, 1.94 mL, 27.5 eq). The mixture was stirred at 60° C. for 2 hrs. The reaction mixture was concentrated under reduced pressure to give a residue. The residue was purified by Prep-HPLC (column: Phenomenex C18 150*25 mm*10 um; mobile phase: [water (NH₄HCO₃)-ACN]; gradient: 15%-45% B over 14 min). Compound 1010 (57.78 mg, 109 μmol, 38.6% yield, 99.6% purity) was obtained as a white solid. LC-MS: (M+H)⁺: 529.3. ¹H NMR: (400 MHz, DMSO-d₆) δ 8.99-8.83 (m, 1H), 8.23 (d, J=8.8 Hz, 1H), 7.69 (d, J=7.6 Hz, 1H), 7.49-7.44 (m, 1H), 7.42-7.36 (m, 2H), 7.34-7.23 (m, 2H), 7.20-7.10 (m, 2H), 7.04-6.96 (m, 2H), 4.89-4.77 (m, 1H), 3.83-3.74 (m, 1H), 3.45-3.34 (m, 3H), 3.18 (d, J=1.2 Hz, 3H), 2.24, (s, 6H), 1.95 (d, J=13.6 Hz, 3H), 1.79 (d, J=5.6 Hz, 3H).

The compounds set forth in Table 25, were prepared using the synthetic procedures of Example 52, or analogous procedures as provided herein.

TABLE 25
Structure and Spectroscopic data for Compounds 1011 to 1032

| No. | Structure |
|---|---|
| 1011 | 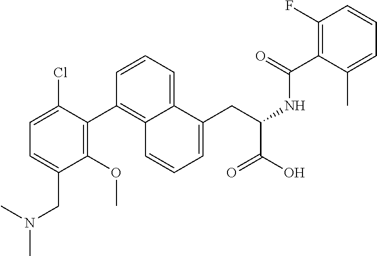 [SMILES: COc1c(CN(C)C)ccc(Cl)c1-c1cccc2c(C[C@H](NC(=O)c3c(C)cccc3F)C(=O)O)cccc12] |
| | m/z = 549.3 |
| 1012* | 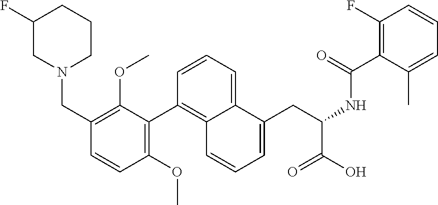 [SMILES: COc1ccc(CN2CCCC(F)C2)c(OC)c1-c1cccc2c(C[C@H](NC(=O)c3c(C)cccc3F)C(=O)O)cccc12] |
| | m/z = 603.2 |
| 1013 | 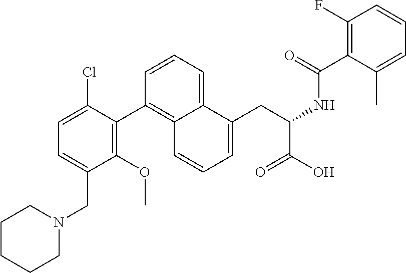 [SMILES: COc1c(CN2CCCCC2)ccc(Cl)c1-c1cccc2c(C[C@H](NC(=O)c3c(C)cccc3F)C(=O)O)cccc12] |
| | m/z = 589.4 |
| 1014 | 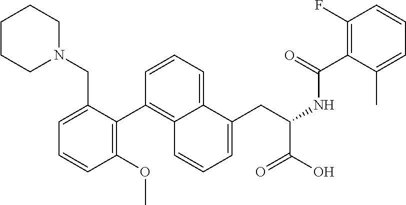 [SMILES: COc1cccc(CN2CCCCC2)c1-c1cccc2c(C[C@H](NC(=O)c3c(C)cccc3F)C(=O)O)cccc12] |
| | m/z = 555.3 |
| 1015* | 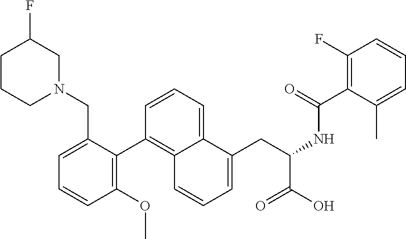 [SMILES: COc1cccc(CN2CCCC(F)C2)c1-c1cccc2c(C[C@H](NC(=O)c3c(C)cccc3F)C(=O)O)cccc12] |
| | m/z = 573.2 |
| 1016 | 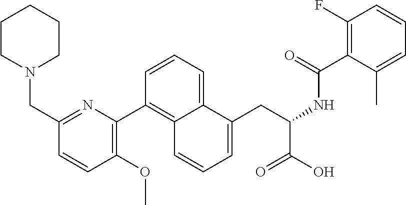 [SMILES: COc1ccc(CN2CCCCC2)nc1-c1cccc2c(C[C@H](NC(=O)c3c(C)cccc3F)C(=O)O)cccc12] |
| | m/z = 556.4 |
| 1017 | 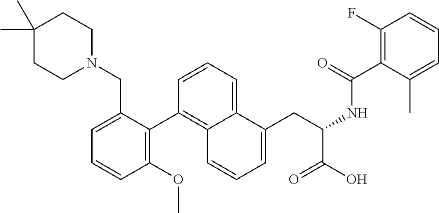 [SMILES: COc1cccc(CN2CCC(C)(C)CC2)c1-c1cccc2c(C[C@H](NC(=O)c3c(C)cccc3F)C(=O)O)cccc12] |
| | m/z = 583.4 |
| 1018 | 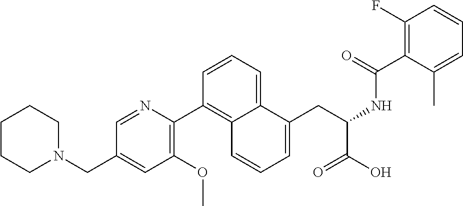 [SMILES: COc1cc(CN2CCCCC2)cnc1-c1cccc2c(C[C@H](NC(=O)c3c(C)cccc3F)C(=O)O)cccc12] |
| | m/z = 556.2 |
| 1019 | 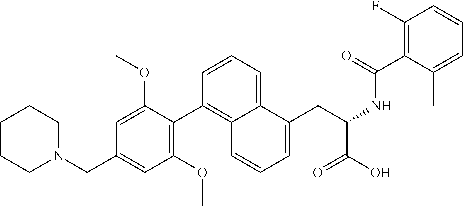 [SMILES: COc1cc(CN2CCCCC2)cc(OC)c1-c1cccc2c(C[C@H](NC(=O)c3c(C)cccc3F)C(=O)O)cccc12] |
| | m/z = 585.3 |
| 1020 | 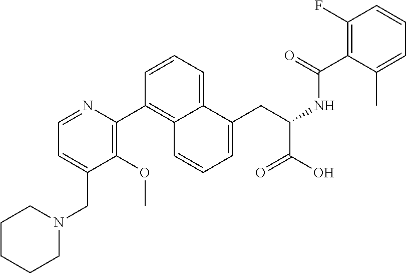 [SMILES: COc1c(CN2CCCCC2)ccnc1-c1cccc2c(C[C@H](NC(=O)c3c(C)cccc3F)C(=O)O)cccc12] |
| | m/z = 556.2 |
| 1021 | 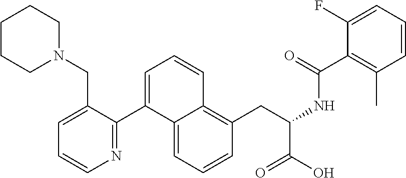 [SMILES: Cc1cccc(F)c1C(=O)N[C@@H](Cc1cccc2c(-c3ncccc3CN3CCCCC3)cccc12)C(=O)O] |
| | m/z = 526.3 |
| 1022 | 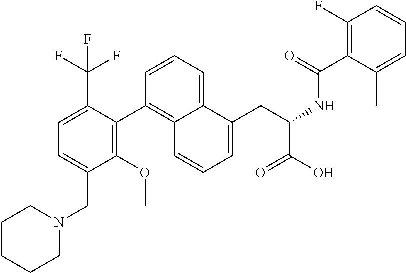 [SMILES: COc1c(CN2CCCCC2)ccc(C(F)(F)F)c1-c1cccc2c(C[C@H](NC(=O)c3c(C)cccc3F)C(=O)O)cccc12] |
| | m/z = 623.4 |
| 1023 | 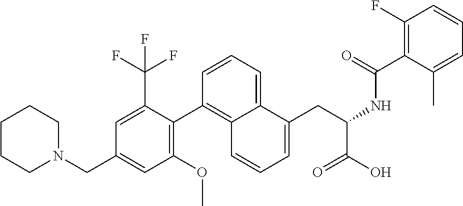 [SMILES: COc1cc(CN2CCCCC2)cc(C(F)(F)F)c1-c1cccc2c(C[C@H](NC(=O)c3c(C)cccc3F)C(=O)O)cccc12] |
| | m/z = 623.4 |
| 1024 | 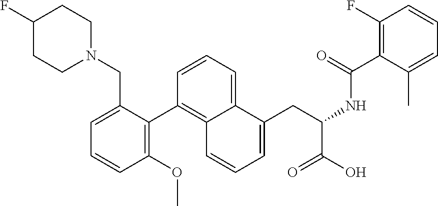 [SMILES: COc1cccc(CN2CCC(F)CC2)c1-c1cccc2c(C[C@H](NC(=O)c3c(C)cccc3F)C(=O)O)cccc12] |
| | m/z = 573.4 |
| 1025 | 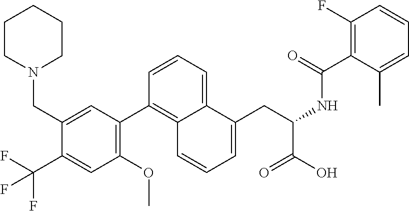 [SMILES: COc1cc(C(F)(F)F)c(CN2CCCCC2)cc1-c1cccc2c(C[C@H](NC(=O)c3c(C)cccc3F)C(=O)O)cccc12] |
| | m/z = 623.1 |
| 1026 | 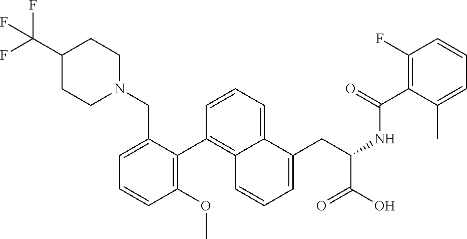 [SMILES: COc1cccc(CN2CCC(C(F)(F)F)CC2)c1-c1cccc2c(C[C@H](NC(=O)c3c(C)cccc3F)C(=O)O)cccc12] |
| | m/z = 623.3 |
| 1027 | 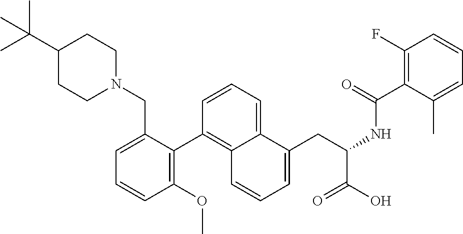 [SMILES: COc1cccc(CN2CCC(C(C)(C)C)CC2)c1-c1cccc2c(C[C@H](NC(=O)c3c(C)cccc3F)C(=O)O)cccc12] |
| | m/z = 611.4 |
| 1028 | 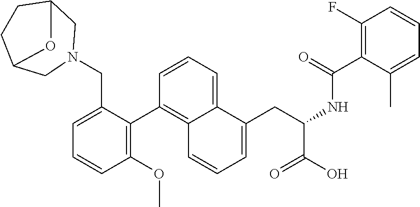 [SMILES: COc1cccc(CN2CC3CCC(C2)O3)c1-c1cccc2c(C[C@H](NC(=O)c3c(C)cccc3F)C(=O)O)cccc12] |
| | m/z = 583.2 |
| 1029 | 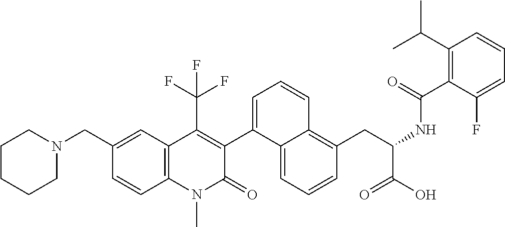 [SMILES: CC(C)c1cccc(F)c1C(=O)N[C@@H](Cc1cccc2c(-c3c(C(F)(F)F)c4cc(CN5CCCCC5)ccc4n(C)c3=O)cccc12)C(=O)O] |
| | m/z = 702.3 |
| 1030 | 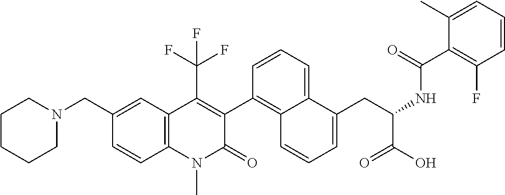 [SMILES: Cc1cccc(F)c1C(=O)N[C@@H](Cc1cccc2c(-c3c(C(F)(F)F)c4cc(CN5CCCCC5)ccc4n(C)c3=O)cccc12)C(=O)O] |
| | m/z = 674.3 |
| 1031 | 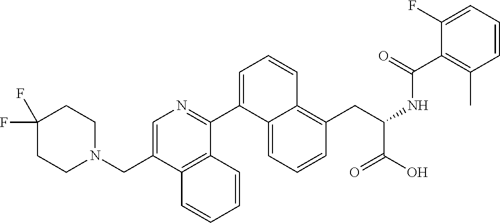 [SMILES: Cc1cccc(F)c1C(=O)N[C@@H](Cc1cccc2c(-c3ncc(CN4CCC(F)(F)CC4)c4ccccc34)cccc12)C(=O)O] |
| | m/z = 612.4 |
| 1032 | 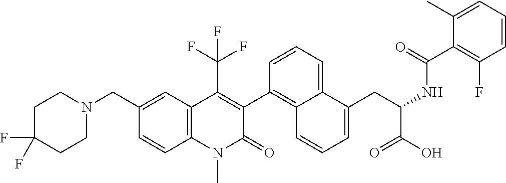 [SMILES: Cc1cccc(F)c1C(=O)N[C@@H](Cc1cccc2c(-c3c(C(F)(F)F)c4cc(CN5CCC(F)(F)CC5)ccc4n(C)c3=O)cccc12)C(=O)O] |
| | m/z = 710.2 |

### Example 53: General Scheme 3C

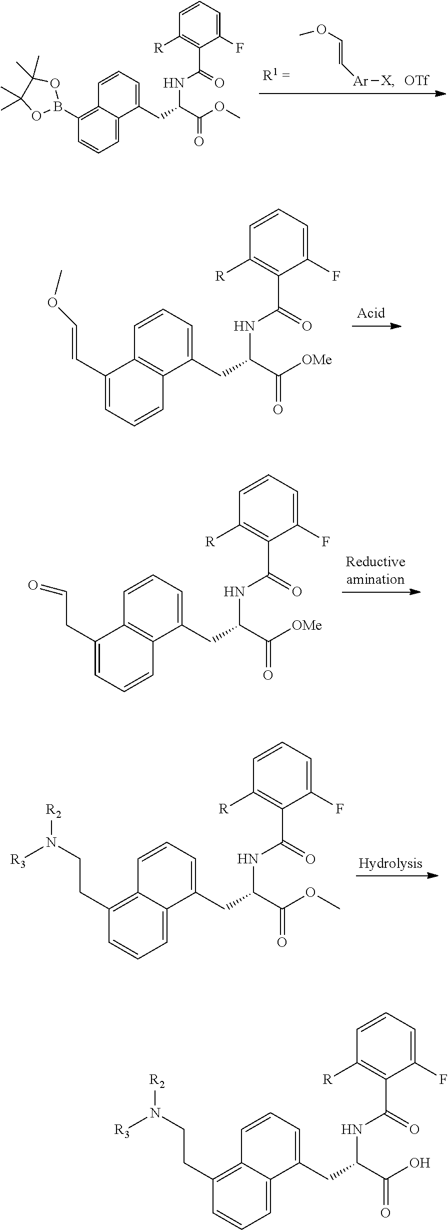

The proceeding compounds were synthesized according to the procedures described in General Scheme 3C using Common Intermediate 3 or Int3 as an intermediate.

### Synthesis of Compound 1033

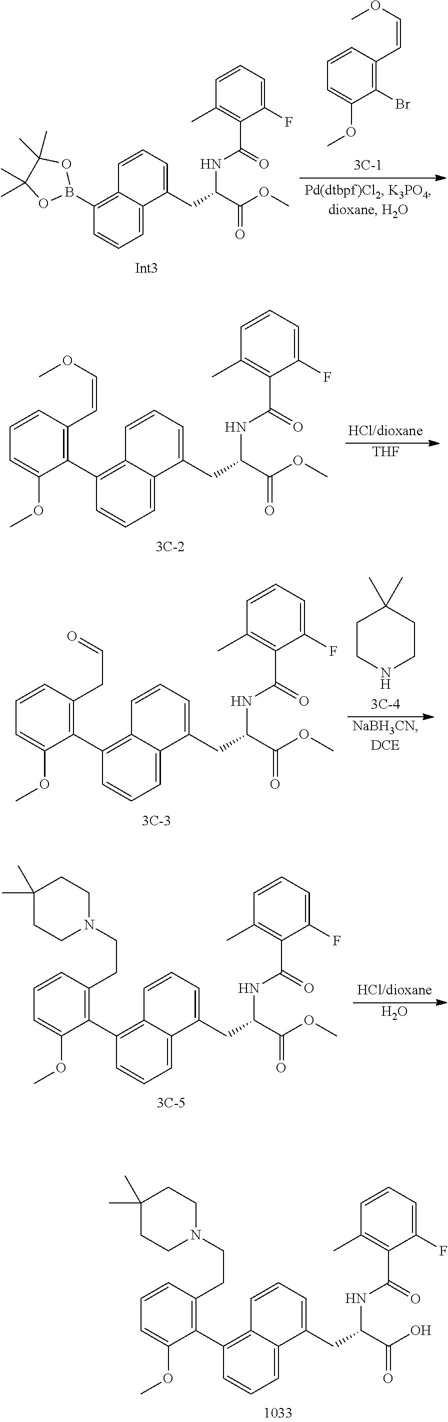

Step 1: To a solution of compound 3C-1 (100 mg, 411 μmol, 1.00 eq) in dioxane (2.00 mL) and H₂O (0.500 mL) was added Pd(dtbpf)Cl₂ (26.8 mg, 41.1 μmol, 0.100 eq), compound Int3 (202 mg, 411 μmol, 1.00 eq) and K₃PO₄ (262 mg, 1.23 mmol, 3.00 eq). The mixture was stirred at 20° C. for 2 hrs. The reaction mixture was diluted with water 20.0 mL, and extracted with EtOAc 60.0 mL (20.0 mL*3). The combined organic layers were washed with brine 40 mL (20 mL*2), dried over Na₂SO₄, filtered, and concentrated under reduced pressure to give a residue. The residue was purified by Prep-TLC (SiO₂, DCM:MeOH=10:1). Compound 3C-2 (200 mg, 379 μmol, 92.1% yield) was obtained as a yellow solid. LC-MS: (M+H)⁺: 528.3. ¹H NMR: (400 MHz, CDCl₃) δ 8.21-8.16 (m, 1H), 7.64-7.61 (m, 1H), 7.41-7.31 (m, 4H), 7.25-7.20 (m, 2H), 7.00-6.83 (m, 4H), 6.43-6.35 (m, 1H), 5.84 (dd, J₁=7.6 Hz, J₂=2.4 Hz, 1H), 5.30-5.25 (m, 1H), 5.16 (dd, J₁=13.2 Hz, J₂=3.6 Hz, 1H), 4.58 (d, J=7.2 Hz, 1H), 3.73-3.58 (m, 11H), 2.33-2.25 (m, 3H).

Step 2: To a solution of compound 3C-2 (150 mg, 284 μmol, 1.00 eq) in THF (4.00 mL) was added HCl/dioxane (4.00 M, 2.00 mL). The mixture was stirred at 20° C. for 2 hrs. The reaction mixture was concentrated under reduced pressure to give a residue. Compound 3C-3 (140 mg, 273 μmol, 95.9% yield) was obtained as a yellow solid. LC-MS: (M+H)⁺: 514.1.

Step 3: To a solution of compound 3C-3 (160 mg, 31.1 μmol, 1.00 eq) in DCE (3.00 mL) was added compound 3C-4 (106 mg, 935 μmol, 3.00 eq). The mixture was stirred at 20° C. for 0.5 hr. And then the mixture was added NaBH₃CN (29.4 mg, 467 μmol, 1.50 eq) and stirred at 20° C. for 2.5 hrs. The reaction mixture was diluted with water 15.0 mL and extracted with DCM 60.0 mL (20.0 mL*3). The combined organic layers were washed with brine 40.0 mL (20.0 mL*2), dried over Na₂SO₄, filtered, and concentrated under reduced pressure to give a residue. The residue was purified by Prep-TLC (SiO₂, DCM:MeOH=10:1). Compound 3C-5 (110 mg, 180 μmol, 57.8% yield) was obtained as a white solid. LC-MS: (M+H)⁺: 611.5. ¹H NMR: δ 8.23 (d, J=8.8 Hz, 1H), 7.68-7.63 (m, 1H), 7.42-7.29 (m, 5H), 7.25-7.20 (m, 1H), 7.15-7.05 (m, 1H), 7.01-6.85 (m, 3H), 6.46-6.44 (m, 1H), 5.31-5.23 (m, 1H), 3.94-3.65 (m, 5H), 3.62 (d, J=4.0 Hz, 3H), 2.76-2.47 (m, 4H), 2.36-2.23 (m, 3H), 1.88-1.52 (m, 4H), 1.44-1.16 (m, 4H), 0.81-0.67 (m, 6H).

Step 4: To a solution of compound 3C-5 (100 mg, 164 μmol, 1.00 eq) in H₂O) (1.00 mL) was added HCl/dioxane (4.00 M, 2.00 mL). The mixture was stirred at 60° C. for 2 hrs. The reaction mixture was concentrated under reduced pressure to give a residue. The residue was purified by Prep-HPLC (column: Phenomenex luna C18 150*25 mm*10 μm; mobile phase: [water (FA)-ACN]; gradient: 20%-50% B over 10 min). Compound 1033 (64.67 mg, 107 μmol, 65.3% yield, 98.7% purity) was obtained as a white solid. LC-MS: (M+H)⁺: 597.2 ¹H NMR: (400 MHz, DMSO-d₆) δ 8.92-8.85 (m, 1H), 8.19 (d, J=9.6 Hz, 1H), 7.67-7.63 (m, 1H), 7.46-7.42 (m, 1H), 7.38 (t, J=8.0 Hz, 1H), 732-7.24 (m, 3H), 7.13 (dd, J₁=8.4 Hz, J₂=3.6 Hz, 1H), 7.03-6.97 (m, 4H), 4.83-4.72 (m, 1H), 3.78-3.69 (m, 1H), 3.55 (d, J=9.6 Hz, 3H), 3.36-3.26 (m, 3H), 2.38-2.25 (m, 4H), 2.22-2.14 (m, 2H), 2.04-1.98 (m, 3H), 1.13-1.03 (m, 4H), 0.76-0.71 (m, 6H).

The compounds set forth in Table 26, were prepared using the synthetic procedures of Example 53, or analogous procedures as provided herein.

TABLE 26
Structure and Spectroscopic data for Compounds 1034 to 1037

| No. | Structure |
|---|---|
| 1034 | 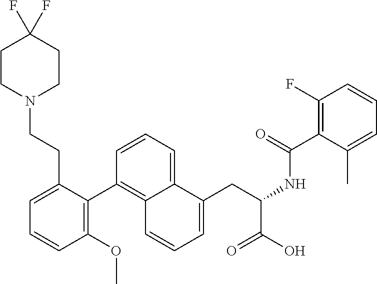 [SMILES: COc1cccc(CCN2CCC(F)(F)CC2)c1-c1cccc2c(C[C@H](NC(=O)c3c(C)cccc3F)C(=O)O)cccc12] |
| | m/z = 605.2 |
| 1035 | 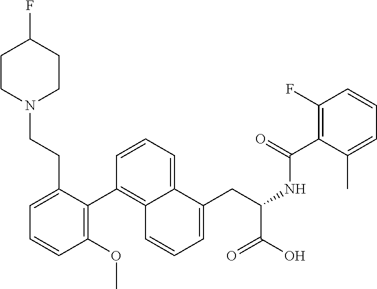 [SMILES: COc1cccc(CCN2CCC(F)CC2)c1-c1cccc2c(C[C@H](NC(=O)c3c(C)cccc3F)C(=O)O)cccc12] |
| | m/z = 587.3 |
| 1036 | 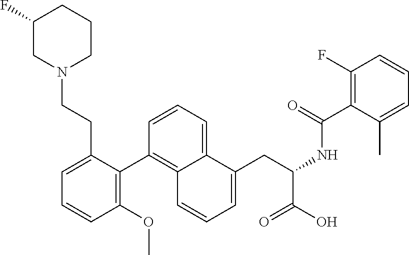 [SMILES: COc1cccc(CCN2CCC[C@@H](F)C2)c1-c1cccc2c(C[C@H](NC(=O)c3c(C)cccc3F)C(=O)O)cccc12] |
| | m/z = 587.3 |
| 1037 | 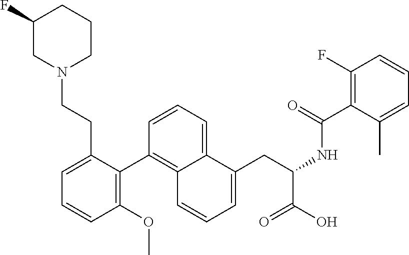 [SMILES: COc1cccc(CCN2CCC[C@H](F)C2)c1-c1cccc2c(C[C@H](NC(=O)c3c(C)cccc3F)C(=O)O)cccc12] |
| | m/z = 587.2 |

### Example 54: Synthesis of Compound 1038

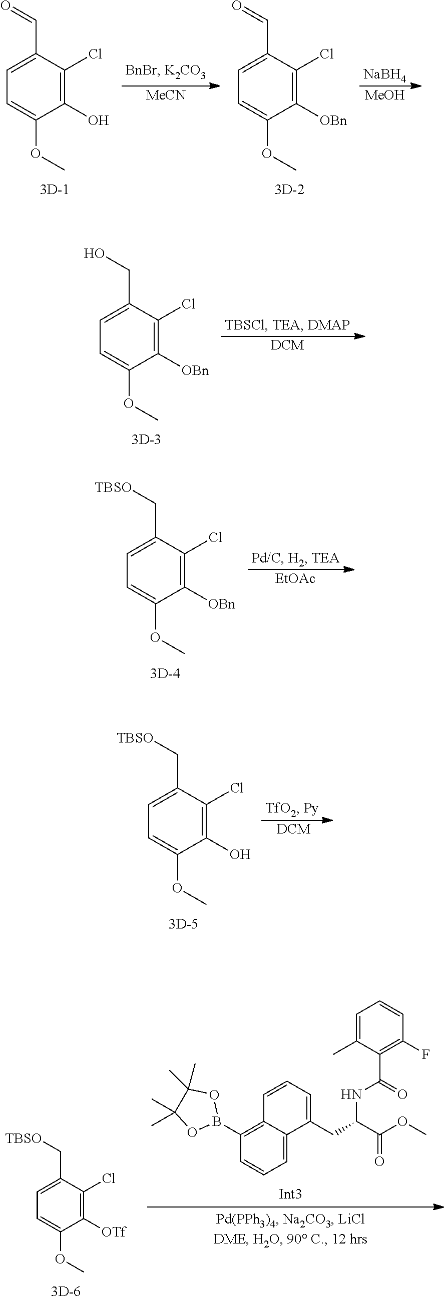

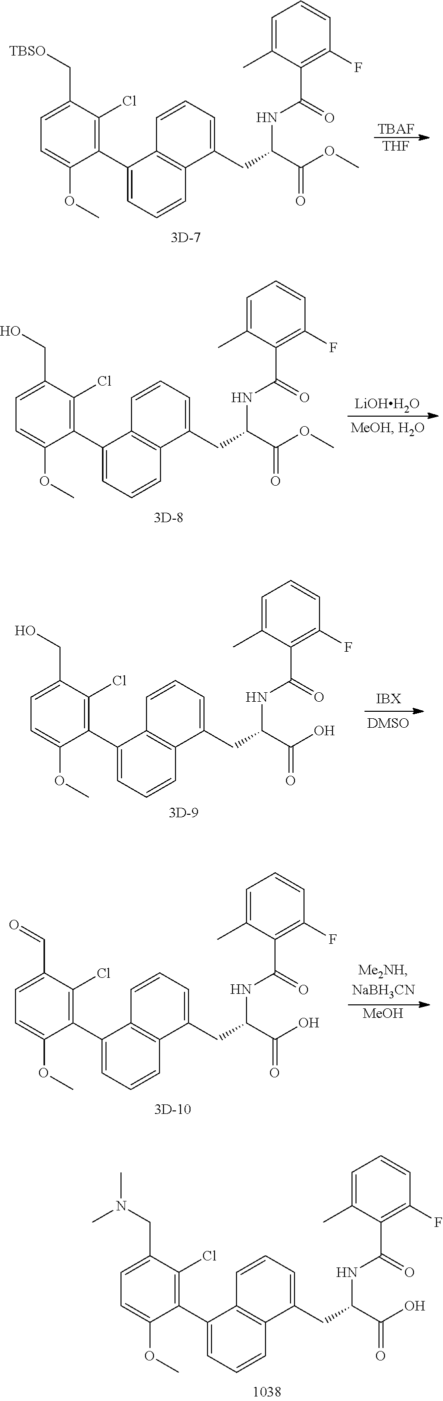

Step 1: To a solution of compound 3D-1 (2.00 g, 10.7 mmol, 1.00 eq) in MeCN (60.0 mL) was added bromomethylbenzene (2.02 g, 11.8 mmol, 1.40 mL, 1.10 eq) and K₂CO₃ (2.97 g, 21.5 mmol, 2.00 eq). The mixture was stirred at 40° C. for 12 hrs. The reaction mixture was concentrated under reduced pressure to remove MeCN. The residue was diluted with H₂O (20.0 mL) and extracted with ethyl acetate (45.0 mL (15.0 mL*3)). The combined organic layers were washed with brine (15.0 mL (5.00 mL*3)), dried over Na₂SO₄, filtered, and concentrated under reduced pressure to give a residue. The residue was purified by column chromatography (SiO₂, Petroleum ether:Ethyl acetate=1:0 to 0:1). Compound 3D-2 (2.71 g, 9.79 mmol, 91.4% yield) was obtained as colorless oil. ¹H NMR: (400 MHz, CDCl₃) δ 10.36 (s, 1H), 7.76 (d, J=8.8 Hz, 1H), 7.58-7.50 (m, 2H), 7.46-7.30 (m, 3H), 6.94 (d, J=8.8 Hz, 1H), 5.07 (s, 2H), 3.96 (s, 3H)

Step 2: To a solution of compound 3D-2 (2.71 g, 9.79 mmol, 1.00 eq) in MeOH (30.0 mL) was added NaBH₄ (1.30 g, 34.4 mmol, 3.51 eq) at 0° C. The mixture was stirred at 0° C. for 1.5 hrs. The residue was diluted with H₂O (20.0 mL) and extracted with ethyl acetate (45.0 mL (15.0 mL*3)). The combined organic layers were washed with brine (10.0 mL), dried over Na₂SO₄, filtered, and concentrated under reduced pressure to give a residue. Compound 3D-3 (2.71 g, 9.72 mmol, 99.3% yield) was obtained as off-white solid. ¹H NMR: (400 MHz, CDCl₃) δ 7.55 (d, J=6.8 Hz, 2H), 7.44-7.30 (m, 3H), 7.17 (d, J=8.8 Hz, 1H), 6.86 (d, J=8.4 Hz, 1H), 5.05 (s, 2H), 4.72 (s, 2H), 3.88 (s, 3H)

Step 3: To a solution of compound 3D-3 (1.00 g, 3.59 mmol, 1.00 eq) in dichloromethane (30.0 mL) was added TBSCl (735 mg, 4.88 mmol, 600 μL, 1.36 eq), TEA (508 mg, 5.03 mmol, 700 μL, 1.40 eq) and DMAP (30.0 mg, 245 μmol, 0.06 eq). The mixture was stirred at 25° C. for 12 hrs. The reaction mixture was concentrated under reduced pressure to remove dichloromethane. The reaction mixture was diluted with H₂O (20.0 mL) and extracted with ethyl acetate (45.0 mL (15.0 mL*3)). The combined organic layers were washed with brine (10.0 mL), dried over Na₂SO₄, filtered, and concentrated under reduced pressure to give a residue. Compound 3D-4 (1.39 g, 3.54 mmol, 98.6% yield) was obtained as colorless oil. LC-MS. (M+H)⁺: 393.1. ¹H NMR: (400 MHz, CDCl₃) δ 7.55 (d, J=7.2 Hz, 2H), 7.42-7.30 (m, 3H), 7.27-7.24 (m, 1H), 6.88 (d, J=8.8 Hz, 1H), 5.04 (s, 2H), 4.74 (s, 2H), 3.87 (s, 3H), 0.97 (s, 9H), 0.13 (s, 6H)

Step 4: To a solution of compound 3D-4 (700 mg, 1.78 mmol, 1.00 eq) in ethyl acetate (10.0 mL) was added Pd/C (100 mg, 10.0% purity) and TEA (273 mg, 2.69 mmol, 375 μL, 1.51 eq) under N₂. The suspension was degassed under vacuum and purged with H₂ several times. The mixture was stirred under H₂ (15 psi) at 25° C. for 0.5 hr. The mixture was filtered, and the filtrate was concentrated under reduced pressure to give a residue. Compound 3D-5 (520 mg, 1.72 mmol, 96.4% yield) was obtained as colorless oil. ¹H NMR: (400 MHz, CDCl₃) δ 7.05 (d, J=8.4 Hz, 1H), 6.82 (d, J=8.4 Hz, 1H), 5.85 (s, 1H), 4.74 (s, 2H), 3.91 (s, 3H), 0.96 (s, 9H), 0.13 (s, 6H)

Step 5: To a solution of compound 3D-5 (520 mg, 1.72 mmol, 1.00 eq) in dichloromethane (20.0 mL) was added Py (2.04 g, 25.8 mmol, 2.08 mL, 15.0 eq) and Tf₂O (1.48 g, 5.25 mmol, 866 μL, 3.06 eq) at 0° C. The mixture was stirred at 25° C. for 1 hr. The reaction mixture was diluted with H₂O (20.0 mL) and extracted with dichloromethane (45.0 mL (15.0 mL*3)). The combined organic layers were washed with brine (10.0 mL), dried over Na₂SO₄, filtered, and concentrated under reduced pressure to give a residue. The residue was purified by Prep-TLC (SiO₂, Petroleum ether:Ethyl acetate=5:1, R_{f}=0.68). Compound 3D-6 (760 mg, crude) was obtained as colorless oil. ¹H NMR: (400 MHz, CDCl₃) δ 7.51 (d, J=8.8 Hz, 1H), 6.99 (d, J=8.8 Hz, 1H), 4.74 (s, 2H), 3.93 (s, 3H), 0.96 (s, 9H), 0.13 (s, 6H)

Step 6: A mixture of compound 3D-6 (300 mg, 690 μmol, 1.19 eq), compound Int3 (309 mg, 580 μmol, 1.00 eq), Pd(PPh₃)₄ (67.0 mg, 57.9 μmol, 0.100 eq), LiCl (45.0 mg, 1.06 mmol, 21.7 μL, 1.83 eq) and Na₂CO₃ (99.0 mg, 934 μmol, 1.61 eq) in DME (6.00 mL) and H₂O (1.50 mL) was degassed and purged with N₂ for 3 times, and then the mixture was stirred at 90° C. for 12 hrs under N₂ atmosphere. The reaction mixture was diluted with H₂O (20.0 mL) and extracted with ethyl acetate (45.0 mL (15.0 mL*3)). The combined organic layers were washed with brine (10.0 mL), dried over Na₂SO₄, filtered, and concentrated under reduced pressure to give a residue. The residue was purified by Prep-HPLC (column: Phenomenex luna C18 150*25 mm*10 μm; mobile phase: [water (FA)-ACN]; gradient: 70%-100% B over 10 min). Compound 3D-7 (90.0 mg, 138 μmol, 23.8% yield) was obtained as colorless oil. LC-MS: (M+H)⁺: 650.4

Step 7: To a solution of compound 3D-7 (90.0 mg, 138 μmol, 1.00 eq) in THF (3.00 mL) was added TBAF (1.00 M, 220 μL, 1.59 eq). The mixture was stirred at 25° C. for 1 hr. The reaction mixture was diluted with aq. NH₄Cl (15.0 mL) and extracted with ethyl acetate (30.0 mL (10.0 mL*3)). The combined organic layers were washed with brine (5.00 mL), dried over Na 2SO₄, filtered, and concentrated under reduced pressure to give a residue. Compound 3D-8 (80.0 mg, crude) was obtained as colorless oil. LC-MS: (M+H)⁺: 536.2

Step 8: To a solution of compound 3D-8 (75.0 mg, 140 μmol, 1.00 eq) in methanol (1.00 mL) and H₂O (1.00 mL) was added LiOH·H₂O (12.0 mg, 286 μmol, 2.04 eq). The mixture was stirred at 25° C. for 1.5 hrs. The reaction mixture was concentrated under reduced pressure to give a residue. Compound 3D-9 (85.0 mg, crude) was obtained as colorless oil. LC-MS: (M+H)⁺: 522.0

Step 9: To a solution of compound 3D-9 (110 mg, 211 μmol, 1.00 eq) in DMSO (2.50 mL) was added IBX (118 mg, 421 μmol, 2.00 eq). The mixture was stirred at 25° C. for 0.5 hr. The reaction mixture was diluted with H₂O (20.0 mL) and extracted with ethyl acetate (45.0 mL (15.0 mL*3)). The combined organic layers were washed with brine (5.00 mL), dried over Na₂SO₄, filtered, and concentrated under reduced pressure to give a residue. The reaction mixture was purified by Prep-TLC (SiO₂, Dichloromethane:Methanol=10:1, R_{f}=0.49) and Prep-TLC (SiO₂, Petroleum ether:Ethyl acetate=1:1, R_{f}=0.06). Compound 3D-10 (85.0 mg, 163 μmol, 77.6% yield) was obtained as colorless oil. LC-MS: (M+H)⁺: 520.1

Step 10: To a solution of compound 3D-10 (85.0 mg, 163 μmol, 1.00 eq) in methanol (2.00 mL) was added Me₂NH (2.00 M, 600 μL, 7.34 eq). The mixture was stirred at 25° C. for 12 hrs. Then NaBH₃CN (17.0 mg, 270 μmol, 1.65 eq) was added, and the mixture was stirred at 25° C. for 1 hr. The reaction mixture was concentrated under reduced pressure to give a residue. The reaction mixture was diluted with H₂O (15.0 mL), then adjust the pH to 7 with aq. HCl (1.00 M), extracted with ethyl acetate (30.0 mL (10.0 mL*3)). The combined organic layers were washed with brine (5.00 mL), dried over Na₂SO₄, filtered, and concentrated under reduced pressure to give a residue. The residue was purified by Prep-HPLC (column: Phenomenex C18 150*25 mm*10 μm; mobile phase: [water (NH₄HCO₃)-ACN]; gradient: 15%-45% B over 14 min). Compound 1038 (29.11 mg, 53.0 μmol, 32.4% yield, 100% purity) was obtained as white solid. LC-MS: (M+H)⁺: 549.3. ¹H NMR: (400 MHz, CD₃CN+D₂O) δ 8.39 (br d, J=8.4 Hz, 1H), 7.68-7.57 (m, 2H), 7.41 (br d, J=6.4 Hz, 1H), 7.31-7.19 (m, 3H), 7.18-7.11 (m, 2H), 6.99-6.86 (m, 2H), 4.89-4.77 (m, 1H), 4.26-4.16 (m, 2H), 3.74 (br dd, J₁=13.6 Hz, J₂=2.4 Hz, 1H), 3.60 (dd, J₁=6.0 Hz, J₂=2.4 Hz, 3H), 3.46-3.31 (m, 1H), 2.71-2.62 (m, 6H), 2.05 (br d, J=11.2 Hz, 3H)

### Example 55: Synthesis of Compound 1039

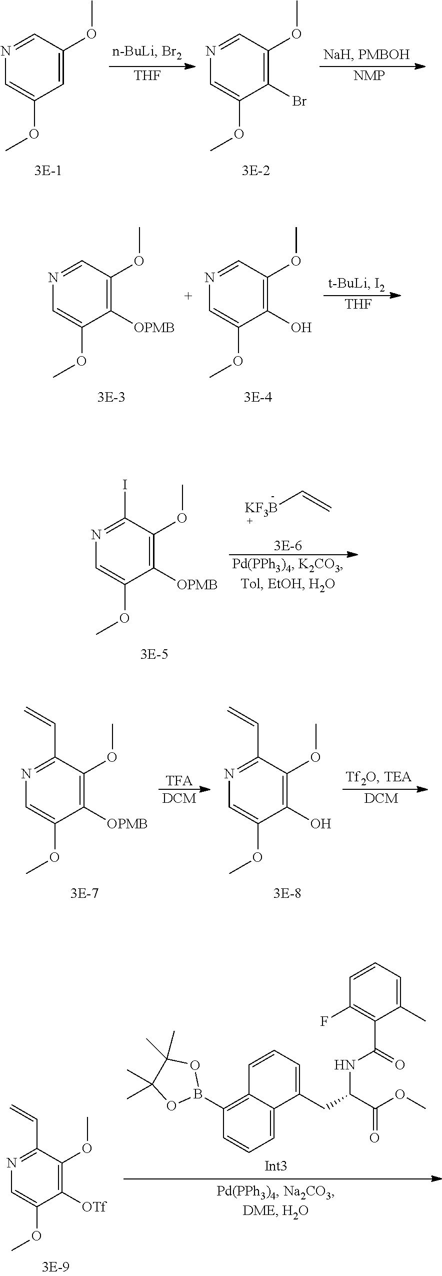

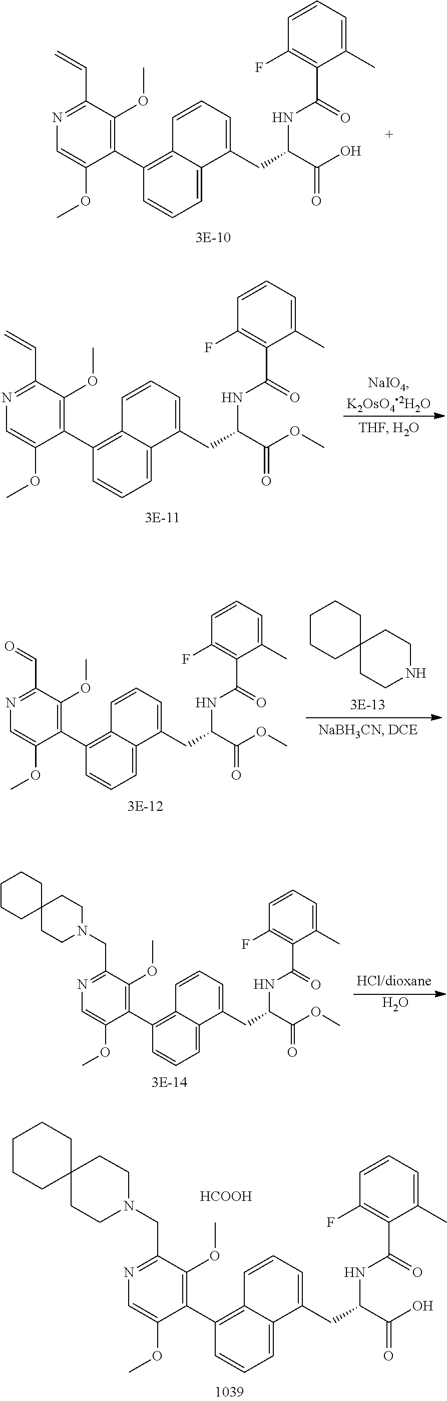

Step 1: To a solution of compound 3E-1 (25.0 g, 180 mmol, 1.00 eq) in dry THF (380 mL) was added n-BuLi (2.50 M, 108 mL, 1.50 eq) at −78° C. During this addition, a precipitate formed, and the reaction was allowed to warm to 20° C. for 30 min. The reaction was then re-cooled to −78° C., and added dropwise Br₂ (57.4 g, 359 mmol, 18.5 mL, 2.0 eq). The mixture was stirred at 20° C. for 2 hrs. The reaction mixture was quenched by addition saturated Na₂SO₃ solution 500 mL at 0° C., and then diluted with water 200 mL and extracted with ethyl acetate 1500 mL (500 mL*3). The combined organic layers were washed with brine 500 mL (500 mL*1), dried over Na₂SO₄, filtered, and concentrated under reduced pressure to give a residue. The residue was triturated with petroleum ether at 20° C. for 20 min, the suspension was filtered, and filtrate was further purified by column chromatography (SiO₂, Petroleum ether:Ethyl acetate=1:0 to 1:1). The filter cake was used next step directly without any purification. Compound 3E-2 (31.9 g, 143 mmol, 79.7% yield, 97.7% purity) was obtained as light-yellow solid. ¹H NMR: (400 MHz, CDCl₃) § 7.96 (s, 2H), 4.00 (s, 6H). LC-MS: (M+H)⁺: 217.9.

Step 2: To a mixture of NaH (4.02 g, 101 mmol, 60.0% purity, 3.00 eq) in dry NMP (110 mL) was added PMBOH (13.9 g, 106 mmol, 12.5 mL, 3.00 eq) dropwise by syringe at 0° C., stirred at 25° C. for 0.5 hr. Then compound 3E-2 (7.48 g, 33.5 mmol, 1.00 eq) was added, the mixture was stirred at 90° C. for 12 hrs. The reaction mixture was quenched by addition saturated NH₄Cl solution 500 mL at 0° C., and then diluted with water 200 mL and extracted with ethyl acetate 1800 mL (600 mL*3). The combined organic layers were washed with brine 500 mL (500 mL*1), dried over Na₂SO₄, filtered, and concentrated under reduced pressure to give a residue. The residue was purified by column chromatography (SiO₂, Petroleum ether:Ethyl acetate=1:0 to 1:1) and reverse-phase HPLC (column: 330 g Flash Coulmn Welch Ultimate XB_C18 20-40 μm 120A; Solvent for sample dissolution about 6.50 grams of sample dissolved in 20 ml of MeOH, Flow rate: 100 ml/min; Mobile phase: H₂O+ACN; Gradient B %: 10-60% 18 min; 50% min; Instrument: ISCO). Compound 3E-3 (820 mg, 2.91 mmol, 2.17% yield, 97.8% purity) was obtained as yellow oil. LC-MS: (M+H)⁻: 276.1. Compound 3E-4 (2.00 g, 12.8 mmol, 9.53% yield, 99.1% purity) was obtained as off-white solid. ¹H NMR: (400 MHz, MeOD) δ 7.57 (s, 2H), 3.81 (s, 6H). LC-MS: (M−H)⁺: 153.9.

Step 3: To a solution of compound 3E-4 (1.80 g, 11.5 mmol, 1.00 eq) and PMBOH (1.59 g, 11.5 mmol, 1.43 mL, 1.00 eq) in dry THF (20.0 mL) was added PPh₃ (4.52 g, 17.3 mmol, 1.50 eq) and DIAD (3.49 g, 17.3 mmol, 3.34 mL, 1.50 eq) at 0° C., the mixture was stirred at 20° C. for 4 hrs. The reaction mixture was diluted with water 60.0 mL and extracted with ethyl acetate 300 mL (100 mL*3). The combined organic layers were washed with brine 60.0 mL (60.0 mL*1), dried over Na₂SO₄, filtered, and concentrated under reduced pressure to give a residue. The residue was purified by column chromatography (SiO₂, Petroleum ether:Ethyl acetate=1:0 to 1:1). Compound 3E-3 (1.90 g, 6.63 mmol, 57.6% yield, 96.0% purity) was obtained as yellow oil. LC-MS: (M+H)⁺: 275.9. ¹H NMR: (400 MHz, MeOD) δ 7.94 (s, 2H), 7.33 (d, J=8.4 Hz, 2H), 6.95-6.82 (m, 2H), 5.12 (s, 2H), 3.90 (s, 6H), 3.77 (s, 3H)

Step 4: To a solution of compound 3E-3 (2.72 g) in dry THF (50.0 mL) was added dropwise t-BuLi (1.30 M, 14.0 mL, 2.74 eq) at −78° C. for 20 mins. A solution of I₂ (3.84 g, 15.1 mmol, 3.05 mL, 2.28 eq) in dry THF (25.0 mL) was added dropwise. The mixture was warmed to 20° C. for 2 hrs. The reaction mixture was quenched by addition Na₂SO₃ solution 150 mL at 0° C., and then extracted with ethyl acetate 300 mL (100 mL*3). The combined organic layers were washed with brine 80.0 mL (80.0 mL*1), dried over Na₂SO₄, filtered, and concentrated under reduced pressure to give a residue. The residue was purified by column chromatography (SiO₂, Petroleum ether:Ethyl acetate=1:0 to 1:1). Compound 3E-5 (1.73 g, 4.00 mmol, 60.4% yield, 92.8% purity) was obtained as yellow oil. ¹H NMR: (400 MHz, MeOD) δ 7.87 (s, 1H), 7.38-7.31 (m, 2H), 6.94-6.86 (m, 2H), 5.17 (s, 2H), 3.92 (s, 3H), 3.82 (s, 3H), 3.79 (s, 3H). LC-MS: (M+H)⁺: 401.8.

Step 5: A mixture of compound 3E-5 (1.23 g, 2.85 mmol, 1.00 eq), compound 3E-6 (457 mg, 3.41 mmol, 1.20 eq), Pd(PPh₃)₄ (329 mg, 285 μmol, 0.100 eq) and K₂CO₃ (1.18 g, 8.54 mmol, 3.00 eq) in Tol (12.0 mL), EtOH (6.00 mL), H₂O (3.00 mL) was degassed and purged with N₂ for 3 times, and then the mixture was stirred at 90° C. for 6 hrs under N₂ atmosphere. The reaction mixture was concentrated under reduced pressure to remove solvent, diluted with water 70.0 mL and extracted with ethyl acetate 150 mL (50.0 mL*3). The combined organic layers were dried over Na₂SO₄, filtered, and concentrated under reduced pressure to give a residue. The residue was purified by column chromatography (SiO₂, Petroleum ether:Ethyl acetate=1:0 to 4:1). Compound 3E-7 (780 mg, 2.59 mmol, 91.0% yield) was obtained as yellow oil. LC-MS: (M+H)⁺: 302.1. ¹H NMR: (400 MHz, CDCl₃) δ 8.05 (s, 1H), 7.41-7.35 (m, 2H), 7.11-6.98 (m, 1H), 6.93-6.87 (m, 2H), 6.27 (dd, J₁=17.2 Hz, J₂=1.6 Hz, 1H), 5.42 (dd, J₁=11.2 Hz, J₂=2.0 Hz, 1H), 5.15 (s, 2H), 3.93 (s, 3H), 3.83 (d, J=9.6 Hz, 6H)

Step 6: To a solution of compound 3E-7 (580 mg, 1.92 mmol, 1.00 eq) in DCM (5.00 mL) was added TFA (921 mg, 8.08 mmol, 0.600 mL, 4.20 eq) at 0° C., the mixture was stirred at 20° C. for 3 hrs. The reaction mixture was concentrated under reduced pressure to give a residue. Compound 3E-8 (330 mg, 1.82 mmol, 94.6% yield) was obtained as yellow oil.

Step 7: To a solution of compound 3E-8 (300 mg, 1.66 mmol, 1.00 eq) and TEA (838 mg, 8.28 mmol, 1.15 mL, 5.00 eq) in DCM (5.00 mL) was added Tf₂O (934 mg, 3.31 mmol, 546 μL, 2.00 eq) at 0° C. The mixture was stirred at 20° C. for 1 hr. The reaction mixture was diluted with water 50.0 mL and extracted with ethyl acetate 150 ml. (50.0 mL*3). The combined organic layers were washed with brine 50.0 mL (50.0 mL*1), dried over Na₂SO₄, filtered, and concentrated under reduced pressure to give a residue. The residue was purified by column chromatography (SiO₂, Petroleum ether:Ethyl acetate=1:0 to 0:1) and Prep-TLC (SiO₂, Petroleum ether:Ethyl acetate=3:1). Compound 3E-9 (140 mg, 422 μmol, 25.5% yield, 94.4% purity) was obtained as yellow oil. LC-MS: (M+H)⁺: 314.1. ¹H NMR: (400 MHz, CDCl₃) δ 8.24 (s, 1H), 7.05-6.93 (m, 1H), 6.40-6.24 (m, 1H), 5.57-5.44 (m, 1H), 4.03 (s, 3H), 3.89 (s, 3H).

Step 8: A mixture of compound 3E-9 (100 mg, 301 μmol, 1.00 eq), compound Int3 (148 mg 301 μmol, 1.00 eq), Pd(PPh₃); (69.7 mg, 60.3 μmol, 0.200 eq) and Na₂CO₃ (95.8 mg, 904 μmol, 3.00 eq) in DME (4.00 mL), H₂O (1.00 mL) was degassed and purged with N₂ for 3 times. The mixture was stirred at 90° C. for 2 hrs under N₂ atmosphere. The reaction mixture was diluted with water 50.0 mL and extracted with ethyl acetate 90.0 mL (30.0 mL*3). The combined organic layers were dried over Na₂SO₄, filtered, and concentrated under reduced pressure to give a residue. The aqueous phase was adjusted pH to 4 with HCl (1.00 M), then extracted with ethyl acetate 90.0 mL (30.0 mL*3). The combined organic layers were washed with brine 30.0 ml. (30 mL*1), dried over Na 2SO₄, filtered, and concentrated under reduced pressure to give compound 3E-11 (165 mg, 246 μmol, 58.2% yield, 76.6% purity). The residue was purified by Prep-TLC (SiO₂, Petroleum ether:Ethyl acetate=1:1). Compound 3E-10 (20.0 mg, 33.2 μmol, 7.87% yield, 87.7% purity) was obtained as yellow oil. LC-MS: (M+H)⁺: 529.2. Compound 3E-11 (165 mg, 246 μmol, 58.2% yield, 76.6% purity) was obtained as yellow oil. LC-MS: (M+H)⁺: 515.2.

Step 9: To a solution of compound 3E-11 (145 mg, 215.9 μmol, 1.00 eq) in MeOH (5.00 mL) was added SOCl₂ (819 mg, 6.88 mmol, 0.500 mL, 31.9 eq), the mixture was stirred at 80° C. for 1 hr. The reaction mixture was concentrated under reduced pressure to remove solvent, diluted with saturated NaHCO₃ solution 30.0 mL and extracted with ethyl acetate 90.0 mL (30.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Petroleum ether:Ethyl acetate=1:1). Compound 3E-10 (135 mg, crude) was obtained as yellow oil. LC-MS: (M+H)⁺: 529.5.

Step 10: To a solution of compound 3E-10 (135 mg, 255 μmol, 1.00 eq) in THF (5.00 mL) and H₂O (5.00 mL) was added NaIO₄ (110 mg, 514 μmol, 28.5 μL, 2.01 eq) and K₂OsO₄·2H₂O (15.0 mg, 40.7 μmol, 0.100 eq) at 0° C., the mixture was stirred at 25° C. for 1 hrs. The residue was diluted with H₂O (20.0 mL) and extracted with ethyl acetate (20.0 mL*3). The combined organic layers were washed with brine mL (20.0 mL*3), dried over Na₂SO₄, filtered, and concentrated under reduced pressure to give a residue. The residue was purified by prep-TLC (SiO₂, Plate 1, Petroleum ether:Ethyl acetate=1:1). Compound 3E-12 (70.0 mg, 132 μmol, 51.66% yield) was obtained as white solid. LC-MS: (M+H)⁺: 531.1

Step 11: To a solution of compound 3E-12 (70.0 mg, 132 μmol, 1.00 eq) in DCE (5.00 mL) was added compound 3E-13 (101 mg, 659 μmol, 4.99 eq), the mixture was stirred at 25° C. for 4 hrs. NaBH₃CN (17.0 mg, 271 μmol, 2.05 eq) was added, and the mixture was stirred at 25° C. for 1 hrs. The residue was diluted with H₂O (20.0 mL) and extracted with ethyl acetate (20.0 mL*3). The combined organic layers were washed with brine (20.0 mL*2), dried over Na₂SO₄, filtered, and concentrated under reduced pressure to give a residue. Compound 3E-14 (80.0 mg, 120 μmol, 90.8% yield) was obtained as yellow oil. LC-MS: (M+H)⁺: 668.2.

Step 12: To a solution of compound 3E-14 (70.0 mg, 105 μmol, 1 eq) in H₂O (1.00 mL) was added HCl/dioxane (4.00 M, 438 μL, 16.7 eq), the mixture was stirred at 60° C. for 2 hrs. The reaction mixture was concentrated under reduced pressure to give a residue. The residue was purified by Prep-HPLC (column: Welch Xtimate C18 150*25 mm*5 um; mobile phase: [water (FA)-ACN]; gradient: 24%-54% B over 10 min). Compound 1039 (25.82 mg, 36.6 μmol, 34.9% yield, 99.1% purity, HCOOH) was obtained as off-white solid. LC-MS: (M+H)⁺: 654.2. ¹H NMR: (400 MHz, DMSO-d₆) δ 9.02-8.85 (m, 1H), 8.31-8.22 (m, 2H), 7.73-7.64 (m, 1H), 7.50-7.39 (m, 2H), 7.38-7.24 (m, 2H), 7.20 (d, J=8.4 Hz, 1H), 7.01 (t, J=8.0 Hz, 2H), 4.86-4.71 (m, 1H), 3.81-3.61 (m, 7H), 3.24 (d, J=8.0 Hz, 3H), 2.58-2.52 (m, 4H), 2.01 (d, J=26.0 Hz, 3H), 1.37 (br s, 10H), 1.30 (br s, 4H).

### Example 56: Synthesis of 1040

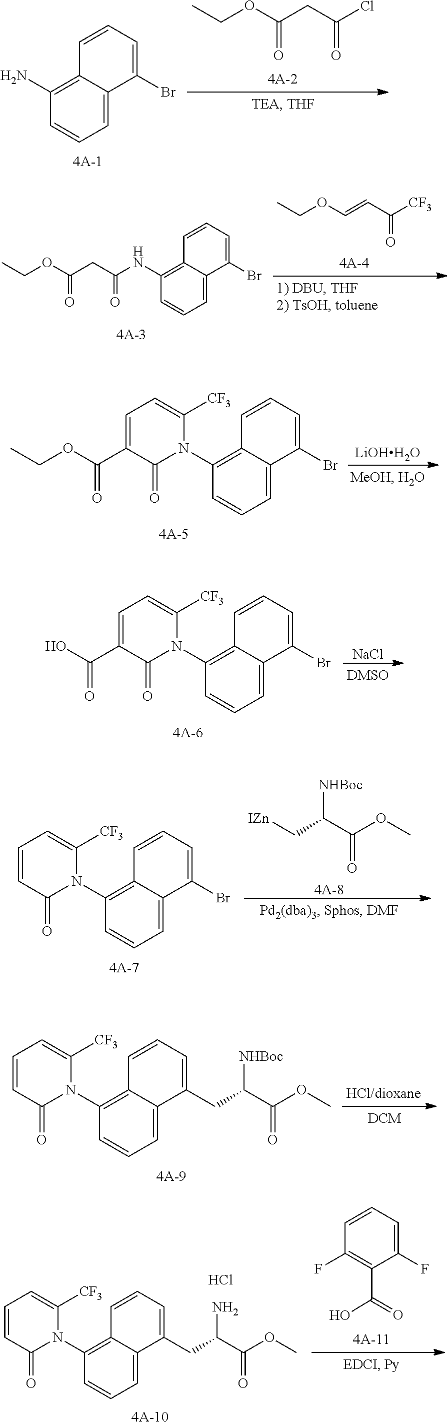

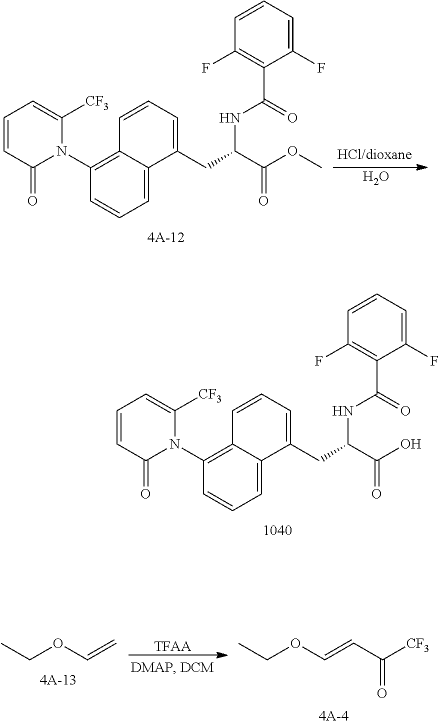

Step 1: To a solution of compound 4A-1 (2.00 g, 9.01 mmol, 1.00 eq) in THF (20.0 mL) was added TEA (2.73 g, 27.0 mmol, 3.76 mL, 3.00 eq) and compound 4A-2 (1.63 g, 10.8 mmol, 1.36 mL, 1.20 eq) at 0° C. The mixture was stirred at 20° C. for 12 hrs. The reaction mixture was diluted with water 50.0 mL and extracted with EtOAc 150 mL (50.0 mL*3). The combined organic layers were washed with brine 150 mL (50.0 mL*3), dried over Na₂SO₄, filtered, and concentrated under reduced pressure to give a residue. The residue was purified by column chromatography (SiO₂, PE/EtOAc=I/O to 3/1). Compound 4A-3 (2.35 g, 6.98 mmol, 77.5% yield) was obtained as a white solid. LC-MS: (M+H)⁺: 337.7. ¹H NMR: (400 MHz, CDCl₃) δ 10.28 (s, 1H), 8.17 (d, J=8.4 Hz, 1H), 8.04 (d, J=8.4 Hz, 1H), 7.93 (d, J=7.2 Hz, 1H), 7.75 (d, J=7.6 Hz, 1H), 7.70-7.66 (m, 1H), 7.49 (t, J=8.4 Hz, 1H), 4.17 (q, J=7.2 Hz, 2H), 3.62 (s, 2H), 1.24 (t, J=7.2 Hz, 3H).

Step 2: To a solution of compound 4A-13 (2.00 g, 27.7 mmol, 2.65 mL, 1.00 eq) in DCM (20.0 mL) was added DMAP (203 mg, 1.66 mmol, 0.0600 eq) and TFAA (6.12 g, 29.1 mmol, 4.05 mL, 1.05 eq) at −10° C. The mixture was stirred at 0° C. for 8 hrs and allowed to warm up at 20° C. and stirred 12 hrs. The mixture was poured into cold aqueous sat. NaHCO₃ (40.0 mL) solution, extracted with DCM 90.0 mL (30.0 mL*3). The combined organic layers were washed with water 60.0 mL (30.0 mL*2) and brine 60.0 mL (30.0 mL*2), dried over Na₂SO₄, filtered, and concentrated under reduced pressure to give a residue. Compound 4A-4 (3.56 g, 21.1 mmol, 76.2% yield) was obtained as a brown oil. LC-MS: (M+H)⁺ 170.9. ¹H NMR: (400 MHz, CDCl₃) δ 7.90 (d, J=12.4 Hz, 1H), 5.85 (d, J=12.4 Hz, 1H), 4.10 (q, J=7.2 Hz, 2H), 1.41 (t, J=7.2 Hz, 3H).

Step 3: To a solution of compound 4A-3 (1.00 g, 2.97 mmol, 1.00 eq) in THF (10.0 mL) was added DBU (475 mg, 3.12 mmol, 471 μL, 1.05 eq) and compound 4A-4 (500 mg, 2.97 mmol, 1.00 eq). The mixture was stirred at 20° C. for 5 hrs. To a residue thus obtained, TsOH (51.2 mg, 297 μmol, 0.100 eq) and toluene (5.00 mL) was added, and the mixture was stirred at 110° C. for 0.5 hr under heating while removing water with a Dean-Stark apparatus. The reaction mixture was diluted with water 20.0 mL and extracted with EtOAc 60.0 mL (20.0 mL*3). The combined organic layers were washed with HCl (1.00 mol) 60.0 ml (20.0 mL*3) and brine 60.0 mL (20.0 mL*3), dried over Na₂SO₄, filtered, and concentrated under reduced pressure to give a residue. The residue was purified by column chromatography (SiO₂, PE/EtOAc=1/0 to 3/1). Compound 4A-5 (940 mg, 1.56 mmol, 52.3% yield, 72.9% purity) was obtained as a yellow oil. LC-MS: (M+H)⁺: 439.9. ¹H NMR: (400 MHz, CDCl₃) δ 8.44 (d, J=8.4 Hz, 1H), 8.32 (d, J=7.2 Hz, 1H), 7.83 (dd, J₁=5.6 Hz, J₂=2.8 Hz, 1H), 7.69 (dd, J₁=8.4 Hz, J₂=7.2 Hz, 1H), 7.48 (d, J=7.6 Hz, 1H), 7.36-7.31 (m, 2H), 6.91 (d, J=7.6 Hz, 1H), 4.37 (q, J=7.2 Hz, 2H), 1.35 (t, J=7.2 Hz, 3H)

Step 4: To a solution of compound 4A-5 (880 mg, 1.46 mmol, 72.9% purity, 1.00 eq) in MeOH (8.00 mL) was added LiOH·H₂O (183 mg, 4.37 mmol, 3.00 eq) and H₂O (4.00 mL). The mixture was stirred at 20° C. for 2 hrs. The reaction mixture was diluted with water 20.0 mL and extracted with EtOAc 30.0 mL (10.0 mL*3). The water layer was adjusted to pH-3 with HCl (1.00 mol) and extracted with EtOAc 30.0 mL (10.0 mL*3). The combined organic layers were washed with brine 30.0 mL (10.0 mL*3), dried over Na₂SO₄, filtered, and concentrated under reduced pressure to give a residue. The residue was purified by Prep-TLC (SiO₂, PE:EtOAc=1:1).

Compound 4A-6 (560 mg, 1.36 mmol, 93.2% yield) was obtained as a yellow solid. LC-MS: (M+H)⁻: 411.8. ¹H NMR: (400 MHz, CDCl₃) δ 8.77-8.51 (m, 2H), 7.95-7.31 (m, 5H), 7.19 (s, 1H).

Step 5: Compound 4A-6 (300 mg, 728 μmol, 1.00 eq) and NaCl (128 mg, 2.18 mmol, 3.00 eq) were taken up into a microwave tube in DMSO (9.00 mL). The sealed tube was heated at 200° C. for 120 min under microwave. The reaction mixture was diluted with water 30.0 mL and extracted with EtOAc 90.0 mL (30.0 mL*3). The combined organic layers were washed with brine 90.0 mL (30 mL*3), dried over Na₂SO₄, filtered, and concentrated under reduced pressure to give a residue. The residue was purified by Prep-TLC (SiO₂, PE:EtOAc=1:1). Compound 4A-7 (240 mg, 652 μmol, 89.5% yield) was obtained as a yellow oil. LC-MS: (M+H)⁺: 367.7

Step 6: To a solution of compound 4A-7 (120 mg, 326 μmol, 1.00 eq) in DMF (2.00 mL) was added SPhos (26.8 mg, 65.2 μmol, 0.200 eq), compound 4A-8 (386 mg, 978 μmol, 3.00 eq) and Pd₂(dba)₃ (29.8 mg, 32.6 μmol, 0.100 eq). The mixture was stirred at 90° C. for 12 hrs. The reaction mixture was diluted with water 20.0 mL and extracted with EtOAc 60.0 mL (20.0 mL*3). The combined organic layers were washed with brine 60.0 mL (20.0 mL*3), dried over Na₂SO₄, filtered, and concentrated under reduced pressure to give a residue. The residue was purified by Prep-TLC (SiO₂, PE:EtOAc=1:1). Compound 4A-9 (90.0 mg, 183 μmol, 56.3% yield, 100% purity) was obtained as a yellow solid. LC-MS: (M-56)⁺: 434.8. ¹H NMR: (400 MHz, CDCl₃) δ 8.25 (d, J=8.8 Hz, 1H), 7.64 (t, J=8.4 Hz, 1H), 7.57 (dd, J₁=9.2 Hz, J₂=7.2 Hz, 1H), 7.46-7.42 (m, 1H), 7.39 (d, J=8.0 Hz, 1H), 7.35-7.29 (m, 2H), 6.93 (d, J=9.6 Hz, 1H), 6.85 (d, J=6.8 Hz, 1H), 5.11 (brs, 1H), 4.75-4.73 (m, 1H), 3.64-3.59 (m, 2H), 3.54 (d, J=18.8 Hz, 3H), 1.43 (d, J=14.8 Hz, 9H),

Step 7: To a solution of compound 4A-9 (90.0 mg, 183 μmol, 100% purity, 1.00 eq) in DCM (1.00 mL) was added HCl/dioxane (4.00 M, 1.00 mL). The mixture was stirred at 20° C. for 2 hrs. The reaction mixture was concentrated under reduced pressure to give a residue. Compound 4A-10 (70.0 mg, 161 μmol, 88.0% yield, 98.5% purity, HCl) was obtained as a yellow solid. LC-MS: (M+H)⁺: 390.9.

Step 8: A solution of compound 4A-10 (60.0 mg, 138 μmol, 98.5% purity, 1.00 eq, HCl) in Py (1.00 mL) was added EDCI (79.6 mg, 415 μmol, 3.00 eq) and compound 4A-11 (26.3 mg, 166 μmol, 1.20 eq). The mixture was stirred at 20° C. for 1 hr. The reaction mixture was diluted with sat. NH₄Cl 10.0 mL and extracted with EtOAc 30.0 mL (10.0 mL*3). The combined organic layers were washed with brine 30.0 mL (10.0 mL*3), dried over Na₂SO₄, filtered, and concentrated under reduced pressure to give a residue. Compound 4A-12 (80.0 mg, crude) was obtained as a yellow solid. LC-MS: (M+H)⁺: 530.9. ¹H NMR: (400 MHz, CDCl₃) δ 7.65 (t, J=7.6 Hz, 1H), 7.57 (dd, J₁=9.2 Hz, J₂=7.2 Hz, 1H), 7.45-7.34 (m, 4H), 7.31-7.28 (m, 2H), 6.97-6.92 (m, 3H), 6.85 (d, J=6.8 Hz, 1H), 6.72-6.68 (m, 1H), 5.26-5.18 (m, 1H), 3.90-3.76 (m, 1H), 3.72-3.63 (m, 1H), 3.55 (d, J=16.8 Hz, 3H).

Step 9: To a solution of compound 4A-12 (70.0 mg, 132 μmol, 1.00 eq) in H₂O (1.00 mL) was added HCl/dioxane (4.00 M, 2.00 mL). The mixture was stirred at 60° C. for 1 hr. The reaction mixture was concentrated under reduced pressure to give a residue. The residue was purified by Prep-HPLC (column: Phenomenex luna C18 150*25 mm*10 um; mobile phase: [water (FA)-ACN]; B %: 29%-59%). Compound 1040 (32.85 mg, 63.6 μmol, 48.2% yield, 100% purity) was obtained as a white solid. LC-MS: (M+H)⁺: 516.9. ¹H NMR: (400 MHz, DMSO-d₆) δ 13.01 (brs, 1H), 9.25-9.22 (m, 1H), 8.32 (d, J=8.4 Hz, 1H), 7.80-7.71 (m, 2H), 7.61-7.59 (m, 1H), 7.52-7.42 (m, 3H), 7.21 (dd, J₁=8.4 Hz, J₂=3.6 Hz, 1H), 7.14-7.07 (m, 3H), 6.89 (dd, J₁=9.2 Hz, J₂=4.8 Hz, 1H), 4.81-4.70 (m, 1H), 7.79-3.71 (m, 1H), 3.44-3.36 (m, 1H).

### Example 57: Synthesis of 1041

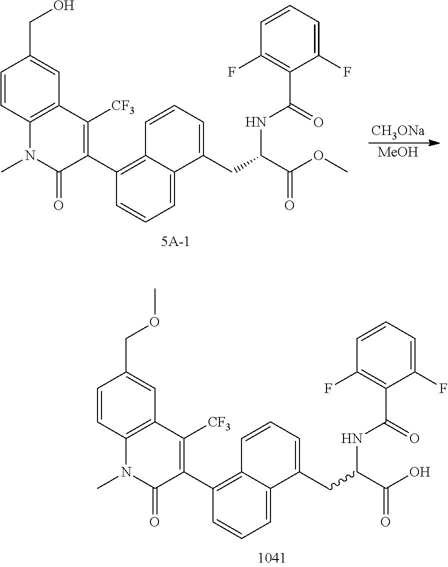

Step 1: To a solution of compound 5A-1 (60.0 mg, 93.3 μmol, 1.00 eq) in MeOH (0.500 mL) was added CH₃ONa (16.8 mg, 93.3 μmol, 30.0% purity, 1.00 eq). The mixture was stirred at 25° C. for 10 hrs. The reaction mixture was adjusted to PH=7 with 1.00 M HCl at 0° C., and then diluted with H₂O 10.0 mL and extracted with ethyl acetate 15.0 mL (5.00 mL*3). The combined organic layers were dried over Na₂SO₄, filtered, and concentrated under reduced pressure to give a residue. The residue was purified by Prep-HPLC (column: Welch Xtimate C18 150*25 mm*5 μm; mobile phase: [water (HCl)-ACN]; B %: 31%-61%, 11 min). Compound 1041 (11.03 mg, 17.4 μmol, 18.6% yield, 98.4% purity) was obtained as an off-white solid. LC-MS: (M+H)⁺: 625.5. ¹H NMR: (400 MHz, DMSO-d₆) δ 13.20-12.58 (m, 1H), 9.46-9.05 (m, 1H), 8.21 (d, J=8.8 Hz, 1H), 7.90-7.81 (m, 1H), 7.76 (s, 2H), 7.68-7.61 (m, 1H), 7.54-7.38 (m, 3H), 7.38-7.30 (m, 2H), 7.18-7.07 (m, 2H), 4.83-4.68 (m, 1H), 4.57 (s, 2H), 3.78-3.69 (m, 4H), 3.43-3.40 (m, 1H), 3.35 (br s, 3H).

### Example 58: Synthesis of Compound 1042

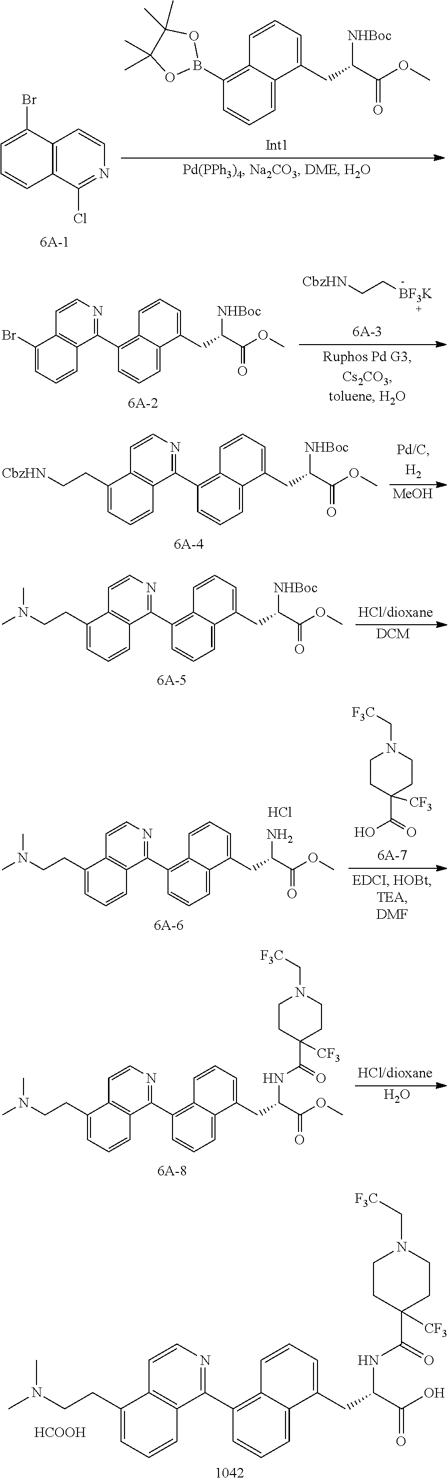

Step 1: To a solution of compound 6A-1 (800 mg, 3.30 mmol, 1.00 eq) in DME (16.0 mL) and H₂O (4.00 mL) was added Pd(PPh₃)₄ (381 mg, 330 μmol, 0.100 eq), compound Int1 (1.63 g, 3.30 mmol, 1.00 eq) and Na₂CO₃ (1.05 g, 9.90 mmol, 3.00 eq). The mixture was stirred at 80° C. for 3 hrs. The reaction mixture was diluted with water 30.0 mL and extracted with EtOAc 90.0 mL (30.0 mL*3). The combined organic layers were washed with brine 60.0 mL (30.0 mL*2), dried over Na₂SO₄, filtered, and concentrated under reduced pressure to give a residue. The residue was purified by column chromatography (SiO₂, PE/EtOAc=I/O to 3/1). Compound 6A-2 (800 mg, 1.49 mmol, 45.3% yield) was obtained as a yellow solid. LC-MS: (M+H)⁺: 535.0. ¹H NMR: (400 MHz, CDCl₃) δ 8.81 (d, J=6.0 Hz, 1H), 8.28 (d, J₁=18.4 Hz, J₂=8.8 Hz, 1H), 8.14 (d, J=6.0 Hz, 1H), 7.98 (d, J=7.2 Hz, 1H), 7.72-7.65 (m, 1H), 7.57 (d, J=8.0 Hz, 2H), 7.31-7.28 (m, 1H), 7.26-7.22 (m, 3H), 5.13-5.01 (m, 1H), 4.81-4.78 (m, 1H), 3.78-3.71 (m, 1H), 3.67 (d, J=12.4 Hz, 3H), 3.58-3.51 (m, 1H), 1.43 (d, J=3.2 Hz, 9H).

Step 2: To a solution of compound 6A-2 (350 mg, 654 μmol, 1.00 eq) in toluene (6.00 mL) was added Ruphos Pd G3 (54.7 mg, 65.4 μmol, 0.100 eq), compound 6A-3 (373 mg, 1.31 mmol, 2.00 eq), H₂O (1.50 mL) and Cs₂CO₃ (639 mg, 1.96 mmol, 3.00 eq). The mixture was stirred at 90° C. for 2 hrs. The reaction mixture was diluted with water 20.0 mL and extracted with EtOAc 60.0 mL (20.0 mL*3). The combined organic layers were washed with brine 60.0 mL (20.0 mL*3), dried over Na₂SO₄, filtered, and concentrated under reduced pressure to give a residue. The residue was purified by column chromatography (SiO₂, PE/EtOAc=1/0 to 1/1). Compound 6A-4 (650 mg, 1.03 mmol, 78.4% yield) was obtained as a yellow solid. LC-MS: (M+H)⁺: 634.6. ¹H NMR (400 MHz, CDCl₃) δ 8.72 (d, J=6.0 Hz, 1H), 8.26 (dd, J₁=18.0 Hz, J₂=8.4 Hz, 1H), 8.01 (d, J=5.6 Hz, 1H), 7.71-7.67 (m, 1H), 7.58-7.56 (m, 1H), 7.54-7.48 (m, 3H), 7.39-7.35 (m, 5H), 7.33-7.29 (m, 2H), 7.24-7.21 (m, 1H), 5.16 (s, 2H), 5.13-5.01 (m, 1H), 4.95 (t, J=4.8 Hz, 1H), 4.81-4.78 (m, 1H), 3.76-3.71 (m, 1H), 3.66 (d, J=14.0 Hz, 3H), 3.63-3.54 (m, 3H), 3.37 (t, J=6.0 Hz, 2H), 1.43 (s, 9H).

Step 3: To a solution of compound 6A-4 (200 mg, 315 μmol, 1.00 eq) in MeOH (2.00 mL) was added Pd/C (100 mg, 10% purity) under N₂. The suspension was degassed under vacuum and purged with H₂ several times. The mixture was stirred under H₂ (15 psi) at 40° C. for 3 hrs. The reaction mixture was diluted with water 10.0 mL and EtOAc 10.0 mL, then adjusted the pH to 5, extracted with EtOAc 20.0 mL (10.0 mL*2). The pH of the aqueous phase was adjusted to 8 and extracted with EtOAc 30.0 ml. (10.0 mL*3). The combined organic layers were washed with brine 20.0 mL (10.0 mL*2), dried over Na₂SO₄, filtered, and concentrated under reduced pressure to give a residue. Compound 6A-5 (102 mg, 193 μmol, 61.2% yield) was obtained as a yellow solid. LC-MS: (M+H)⁺: 528.1. ¹H NMR: (400 MHz, CDCl₃) δ 8.73 (d, J=6.0 Hz, 1H), 8.25 (dd, J₁=17.2 Hz, J₂=8.0 Hz, 1H), 7.95 (d, J=6.0 Hz, 1H), 7.71-7.65 (m, 1H), 7.57 (d, J=7.2 Hz, 2H), 7.46 (d, J=8.8 Hz, 1H), 7.34-7.30 (m, 2H), 7.26-7.19 (m, 2H), 5.12-5.00 (m, 1H), 4.81-4.76 (m, 1H), 3.78-3.72 (m, 1H), 3.66 (d, J=14.8 Hz, 3H), 3.60-3.52 (m, 1H), 3.37-3.33 (m, 2H), 2.78-2.74 (m, 2H), 2.45 (s, 6H), 1.43 (s, 9H).

Step 4: To a solution of compound 6A-5 (102 mg, 193 μmol, 1.00 eq) in DCM (1.00 mL) was added HCl/dioxane (4.00 M, 2.00 mL). The mixture was stirred at 20° C. for 1 hr. The reaction mixture was concentrated under reduced pressure to give a residue. Compound 6A-6 (85.0 mg, 183 μmol, 94.8% yield, HCl) was obtained as a yellow solid. LC-MS: (M+H)⁺: 428.2.

Step 5: To a solution of compound 6A-6 (80.0 mg, 172 μmol, 1.00 eq, HCl) in DMF (2.00 mL) was added EDCI (66.1 mg, 345 μmol, 2.00 eq), compound 6A-7 (57.8 mg, 207 μmol, 1.20 eq), TEA (87.2 mg, 862 μmol, 120 μL, 5.00 eq) and HOBt (46.6 mg, 345 μmol, 2.00 eq). The mixture was stirred at 20° C. for 3 hrs. The reaction mixture was diluted with water 20.0 mL and extracted with EtOAc 60.0 mL (20.0 mL*3). The combined organic layers were washed with brine 60.0 mL (20.0 mL*3), dried over Na₂SO₄, filtered, and concentrated under reduced pressure to give a residue. The residue was purified by Prep-TLC (SiO₂, DCM:MeOH=10:1). Compound 6A-8 (80.0 mg, 116 μmol, 67.4% yield) was obtained as a yellow solid. LC-MS: (M+H)⁺: 689.2. ¹H NMR: (400 MHz, CDCl₃) δ 8.73 (d, J=6.0 Hz, 1H), 8.31-8.24 (m, 1H), 8.00 (d, J₁=5.6 Hz, J₂=2.0 Hz, 1H), 7.74-7.69 (m, 1H), 7.60-7.56 (m, 2H), 7.47-7.43 (m, 1H), 7.35-7.28 (m, 3H), 7.25-7.21 (m, 1H), 6.39-6.32 (m, 1H), 5.13-5.07 (m, 1H), 3.78-3.60 (m, 5H), 3.47-3.45 (m, 2H), 3.30-2.96 (m, 4H), 2.88-2.83 (m, 2H), 2.77-2.72 (m, 1H), 2.66 (s, 6H), 2.62-2.56 (m, 1H), 2.41-2.27 (m, 4H).

Step 6: To a solution of compound 6A-8 (70.0 mg, 102 μmol, 1.00 eq) in H₂O (1.00 mL) was added HCl/dioxane (4.00 M, 1.00 mL). The mixture was stirred at 60° C. for 3 hrs. The reaction mixture was concentrated under reduced pressure to give a residue. The residue was purified by Prep-HPLC (column: Welch Ultimate C18 150*25 mm*5 μm; mobile phase: [water (FA)-ACN]; B %: 10%-40%, 10 min). Compound 1042 (32.27 mg, 47.5 μmol, 46.7% yield, 99.3% purity, HCOOH) was obtained as a white solid. LC-MS: (M+H)⁺: 675.3. ¹H NMR: (400 MHz, DMSO-d₆) δ 8.67 (d, J=6.0 Hz, 1H), 8.37-8.32 (m, 2H), 8.18 (br s, 1H), 8.06 (d, J=6.0 Hz, 1H), 7.77-7.72 (m, 1H), 7.66 (d, J=7.2 Hz, 1H), 7.54 (d, J=6.8 Hz, 1H), 7.44-7.36 (m, 2H), 7.27-7.20 (m, 2H), 7.02 (d, J=8.0 Hz, 1H), 4.83-4.75 (m, 1H), 3.85-3.81 (m, 1H), 3.55-3.52 (m, 1H), 2.98-2.89 (m, 2H), 2.88-2.75 (m, 5H), 2.40 (s, 6H), 2.28-1.99 (m, 4H), 1.88-1.70 (m, 1H), 1.62-1.56 (m, 2H).

### Example 58: Synthesis of Compound 1043

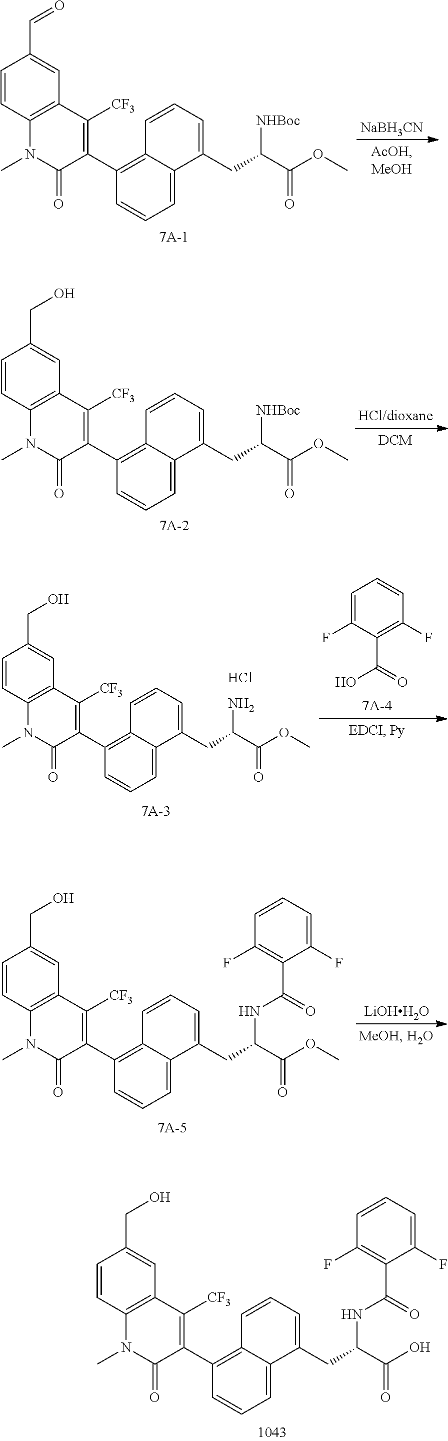

Step 1: To a solution of compound 7A-1 (500 mg, 858 μmol, 1.00 eq) in MeOH (5.00 mL) was added AcOH (2.00 mL) and NaBH₃CN (108 mg, 1.72 mmol, 2.00 eq). The mixture was stirred at 25° C. for 2 hrs. The reaction mixture was diluted with H₂O 50.0 mL and extracted with EtOAc 45.0 mL (15.0 mL*3). The combined organic layers were dried over Na₂SO₄, filtered, and concentrated under reduced pressure to give a residue. The residue was purified by column chromatography (SiO₂, Petroleum ether/Ethyl acetate=3/1 to 1/1). Compound 7A-2 (262 mg, 448 μmol, 52.2% yield, N/A purity) was obtained as a yellow solid. LC-MS: (M−H)⁺: 529.1. ¹H NMR: (400 MHz, DMSO-d6) δ 8.14-8.12 (t, J=8.0 Hz 1H), 7.89 (s, 1H), 7.73 (s, 2H), 7.47-7.38 (m, 1H), 7.35-7.33 (m, 5H), 5.44 (s, 1H), 4.65-4.64 (d, J=4.0 Hz, 2H), 4.35-4.32 (m, 1H), 3.71 (s, 3H), 3.62-3.56 (d, J=11.2 Hz, 4H), 3.33 (s, 1H), 1.33-1.08 (m, 9H).

Step 2: To a solution of compound 7A-2 (100 mg, 171 μmol, 1.00 eq) in DCM (2.00 mL) was added HCl/dioxane (4.00 M, 2.00 mL, 46.7 eq). The mixture was stirred at 25° C. for 2 hrs. The mixture was stirred at 25° C. for 0.5 hr. The reaction mixture was concentrated under reduced pressure to give a residue. Compound 7A-3 (75.0 mg, crude, HCl) was obtained as a yellow solid. LC-MS: (M+H)⁺: 485.3.

Step 3: To a compound 7A-3 (60.0 mg, 115 μmol, 1.00 eq, HCl) and compound 7A-4 in DCM (2.00 mL) was added DIEA (44.7 mg, 345 μmol, 60.2 μL, 3.00 eq) and HATU (87.6 mg, 230 μmol, 2.00 eq). The mixture was stirred at 25° C. for 1 hr. The reaction mixture was diluted with water 5.00 mL and extracted with ethyl acetate 6.00 mL (2.00 mL*3). The combined organic layers concentrated under reduced pressure to give a residue. The residue was purified by Prep-TLC (SiO₂, Petroleum ether/Ethyl acetate=3:2, R_{f}=0.34). Compound 7A-5 (68.0 mg, 109 μmol, 94.5% yield, N/A purity) was obtained as a yellow solid. LC-MS: (M+H)⁺: 625.1. ¹H NMR: (400 MHz, DMSO-d6). δ 9.42 (dd, J₁=11.8 Hz, J₂=5.8 Hz, 1H), 8.16 (dd, J₁=4.4 Hz, J₂=4.0 Hz, 1H), 7.90 (s, 1H), 7.73 (s, 2H), 7.51-7.40 (m, 1H), 7.53-7.32 (m, 5H), 7.15-7.14 (m, 2H), 5.43 (t, J=5.6 Hz, 1H), 4.82-4.79 (m, 1H), 4.73-4.56 (d, J=2.8 Hz, 2H), 3.69-3.61 (m, 4H), 3.66-3.60 (m, 3H), 3.54-3.51 (m, 1H).

Step 4: To a solution of compound 7A-5 (80.0 mg, 114 μmol, 1.00 eq) in MeOH (1.00 mL) was added LiOH·H₂O (7.21 mg, 172 μmol, 1.50 eq) in H₂O (0.500 mL). The mixture was stirred at 25° C. for 1 hr. The reaction mixture was concentrated under reduced pressure to give a residue. The residue was purified by Prep-HPLC (column: Phenomenex C18 150*25 mm*10 μm; mobile phase: [water (NH₄HCO₃)-ACN]; B %: 10%-40%, 8 min). Compound 1043 (21.51 mg, 34.4 μmol, 39.0% yield, 97.7% purity) was obtained as a white solid. LC-MS: (M+H)⁺: 611.4. ¹H NMR: (400 MHz, DMSO-d6) δ 8.82-8.80 (m, 1H), 8.29 (t, J=7.2 Hz, 1H), 7.89 (s, 1H), 7.73 (s, 2H), 7.63-7.58 (m, 1H), 7.51-7.41 (s, 2H), 7.36-7.27 (m, 3H), 7.11 (t, J=9.4 Hz, 2H), 5.44 (s, 1H), 4.68 (s, 3H), 3.77-3.69 (m, 4H), 3.39-3.37 (m, 1H).

### Example 59: Synthesis of Compound 1044

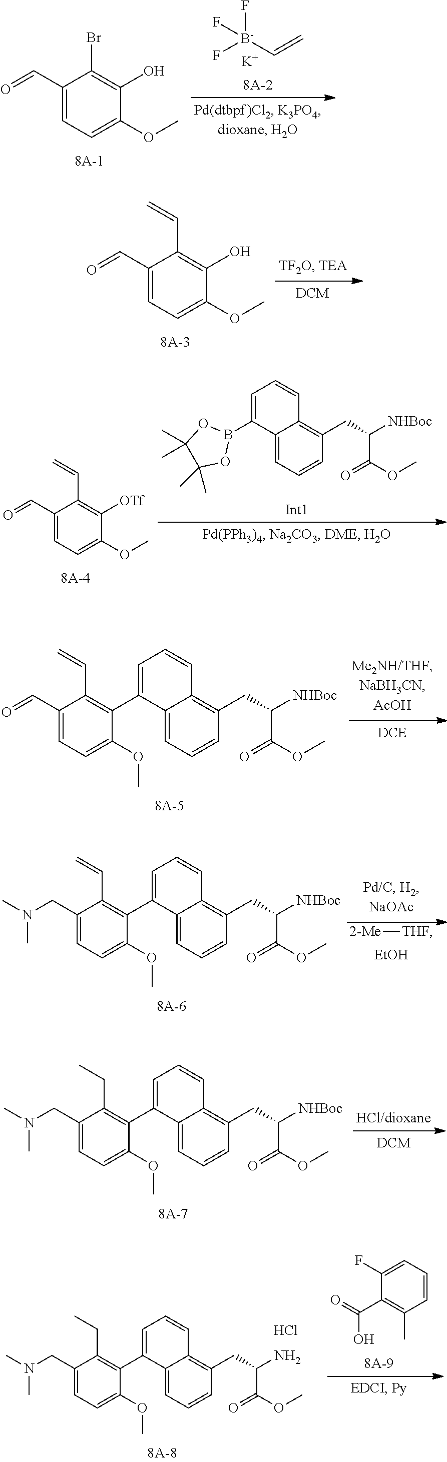

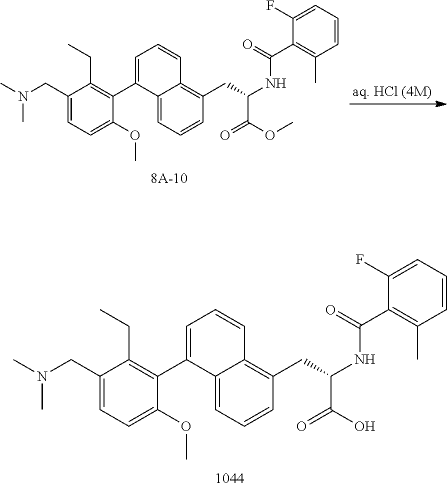 [SMILES: CCc1c(CN(C)C)ccc(OC)c1-c1cccc2c(C[C@H](NC(=O)c3c(C)cccc3F)C(=O)OC)cccc12 ; CCc1c(CN(C)C)ccc(OC)c1-c1cccc2c(C[C@H](NC(=O)c3c(C)cccc3F)C(=O)O)cccc12]

Step 1: A mixture of compound 8A-1 (5.00 g, 21.6 mmol, 1.00 eq), compound 8A-2 (7.25 g, 54.1 mmol, 2.50 eq), ditertbutyl(cyclopentyl)phosphanedichloropalladium; iron (1.41 g, 2.16 mmol, 0.100 eq) and K₃PO₄ (13.7 g, 64.9 mmol, 3.00 eq) in dioxane (50.0 mL) and H₂O (10.0 mL) was degassed and purged with N₂ for 3 times, and then the mixture was stirred at 60° C. for 6 hrs under N₂ atmosphere. The reaction mixture was diluted with H₂O (100 mL) and extracted with ethyl acetate (100 mL*3). The organic layer was washed with brine (100 mL*1). Then the organic layer was dried over Na₂SO₄ and concentrated under reduced pressure to give residue. The residue was purified by column chromatography (SiO₂, Petroleum ether:Ethyl acetate=1:0 to 3:1), TLC (Petroleum ether:Ethyl acetate=1:1, R_{f}=0.78). Compound 3 (3.60 g, 20.2 mmol, 93.3% yield) was obtained as a yellow solid. LC-MS: (M+H)⁺: 179.2. ¹H NMR: (400 MHz, DMSO-d₆) δ 9.99 (s, 1H), 9.17 (s, 1H), 7.39 (d, J=8.4 Hz, 1H), 7.21-7.06 (m, 2H), 5.70-5.54 (m, 2H), 3.91 (s, 3H).

Step 2: To a solution of compound 8A-3 (700 mg, 3.93 mmol, 1.00 eq) in DCM (6.00 mL) was added Tf₂O (3.33 g, 11.8 mmol, 1.94 mL, 3.00 eq) and TEA (2.39 g, 23.5 mmol, 3.28 mL, 6.00 eq) at −40° C. The mixture was stirred at −40° C. for 2 hrs. The reaction mixture was concentrated under reduced pressure to give a residue. The residue was purified by Prep-TLC (Petroleum ether:Ethyl acetate=3:1, R_{f}=0.52). Compound 8A-4 (900 mg, 2.90 mmol, 73.8% yield) was obtained as a yellow oil. LC-MS: (M+H)⁺: 311.0. ¹H NMR: (400 MHz, DMSO-d₆) δ 10.01 (s, 1H), 7.98 (d, J=8.8 Hz, 1H), 7.47 (d, J=8.8 Hz, 1H), 7.05-6.97 (m, 1H), 5.89 (dd, J₁=11.6 Hz, J₂=1.2 Hz, 1H), 5.62 (dd, J₁=18.0 Hz, J₂=1.2 Hz, 1H), 4.00 (s, 3H).

Step 3: A mixture of compound 8A-4 (900 mg, 2.90 mmol, 1.00 eq), compound Int1 (1.32 g 2.90 mmol, 1.00 eq), Na₂CO₃ (922 mg, 8.70 mmol, 3.00 eq) and Pd(PPh₃)₄ (335 mg, 290 μmol, 0.100 eq) in DME (10.0 mL) and H₂O (2.00 mL) was degassed and purged with N₂ for 3 times, and then the mixture was stirred at 90° C. for 2 hrs under N₂ atmosphere. The reaction mixture was concentrated under reduced pressure to give a residue, then the residue was diluted with H₂O (40.0 mL) and extracted with ethyl acetate (40.0 mL*3). The organic layer was washed with brine (40.0 mL*1), dried over Na₂SO₄ and concentrated under reduced pressure to give residue. The residue was purified by column chromatography (SiO₂, Petroleum ether:Ethyl acetate=1:0 to 5:1). Compound 8A-5 (1.00 g, 2.04 mmol, 70.4% yield) was obtained as a yellow oil. LC-MS: (M-55)⁺: 434.1

Step 4: To a solution of compound 8A-5 (400 mg, 817 μmol, 1.00 eq), N-methylmethanamine (2.00 M, 4.09 mL, 10.0 eq) in DCE (4.00 mL) was added AcOH (9.81 mg, 163 μmol, 9.35 μL, 0.200 eq) and stirred at 25° C. for 2 hrs. Then NaBH₃CN (102 mg, 1.63 mmol, 2.00 eq) was added and stirred at 25° C. for 2 hrs. The reaction mixture was diluted with H₂O (30.0 mL) and extracted with DCM (30.0 mL*3). The organic layer was washed with brine (30.0 mL*1), dried over Na₂SO₄ and concentrated under reduced pressure to give residue. The residue was purified by Prep-TLC (DCM:MeOH=10:1, R_{f}=0.49). Compound 8A-6 (300 mg, 578 μmol, 70.8% yield) was obtained as a yellow oil. LC-MS: (M+H)⁺: 519.5. ¹H NMR: (400 MHz, DMSO-d₆) δ 8.04-8.01 (m, 1H), 7.65-7.54 (m, 1H), 7.50-7.41 (m, 2H), 7.38-7.35 (m, 1H), 7.31-7.20 (m, 2H), 7.14 (d, J=8.4 Hz, 1H), 7.07-7.05 (m, 1H), 6.30-6.21 (m, 1H), 5.09-4.86 (m, 2H), 4.39-4.33 (m, 1H), 3.66-3.51 (m, 8H), 2.37 (d, J=5.6 Hz, 8H), 1.31 (d, J=2.4 Hz, 9H).

Step 5: To a solution of compound 8A-6 (250 mg, 482 μmol, 1.00 eq) in 2-Me-THF (3.00 mL) and EtOH (1.00 mL) was added NaOAc (118 mg, 1.45 mmol, 3.00 eq) and Pd/C (30.0 mg, 10.0% purity) under N₂ atmosphere. The suspension was degassed and purged with H₂ for 3 times. The mixture was stirred under H₂ (15 Psi) at 40° C. for 12 hrs. The reaction mixture was filtered, and the filtrate was concentrated under the vacuum. The residue was purified by Prep-TLC (SiO₂, DCM:MeOH=10:1, R_{f}=0.49). Compound 8A-7 (150 mg, 288 μmol, 59.7% yield) was obtained as a yellow oil. LC-MS: (M+H)⁺: 521.3.

Step 6: To a solution of compound 8A-7 (130 mg, 249 μmol, 1.00 eq) in DCM (2.00 mL) was added HCl/dioxane (4.00 M, 0.500 mL, 8.01 eq). The mixture was stirred at 25° C. for 2 hrs. The reaction mixture was concentrated under reduced pressure to give a residue. Compound 8A-8 (120 mg, crude, HCl) was obtained as a yellow oil. LC-MS: (M+H)⁺: 421.2.

Step 7: To a solution of compound 8A-8 (120 mg, 262 μmol, 1.00 eq, HCl), compound 8A-9 (40.5 mg, 262 μmol, 1.00 eq) in Py (2.00 mL) was added EDCI (151 mg, 787 μmol, 3.00 eq). The mixture was stirred at 25° C. for 2 hrs. The reaction mixture was diluted with H₂O (30.0 mL) and extracted with DCM (30.0 mL*3). The organic layer was washed with brine (30.0 mL*1), dried over Na₂SO₄ and concentrated under reduced pressure to give residue. The residue was purified by Prep-TLC (SiO₂, DCM:MeOH=10:1, R_{f}=0.45). Compound 8A-10 (90.0 mg, 162 μmol, 61.5% yield) was obtained as a yellow oil. LC-MS: (M+H)⁺: 557.3

Step 8: A solution of compound 8A-0 (80.0 mg, 143 μmol, 1.00 eq) in HCl (4.00 M, 1.00 mL, 27.8 eq) was stirred at 60° C. for 1 hr. The reaction mixture was concentrated under reduced pressure to give a residue. The residue was purified by Prep-HPLC (column: Phenomenex C18 150*25 mm*10 μm; mobile phase: [water (NH₄HCO₃)-ACN]; gradient: 13%-43% B over 10 min). Compound 1044 (59.23 mg, 109 μmol, 75.8% yield, 99.8% purity) was obtained as a white solid. LC-MS: (M+H)⁺: 543.3. ¹H NMR: (400 MHz, DMSO-d₆) δ 8.97 (t, J=9.2 Hz, 1H), 8.17 (d, J=8.0 Hz, 1H), 7.64 (t, J=7.6 Hz, 1H), 7.44 (t, J=6.8 Hz, 1H), 7.36-7.24 (m, 4H), 7.13 (dd, J₁=8.4 Hz, J₂=2.8 Hz, 1H), 7.05-6.91 (m, 3H), 4.87-4.76 (m, 1H), 3.76 (dd, J₁=14.4 Hz, J₂=3.6 Hz, 1H), 3.52 (d, J=10.4 Hz, 3H), 3.47-3.35 (m, 3H), 2.46-2.42 (m, 1H), 2.20 (d, J=2.4 Hz, 6H), 2.12-2.04 (m, 1H), 2.03-1.93 (m, 3H), 0.75-0.69 (m, 3H).

### Example 60: Synthesis of Compound 1045

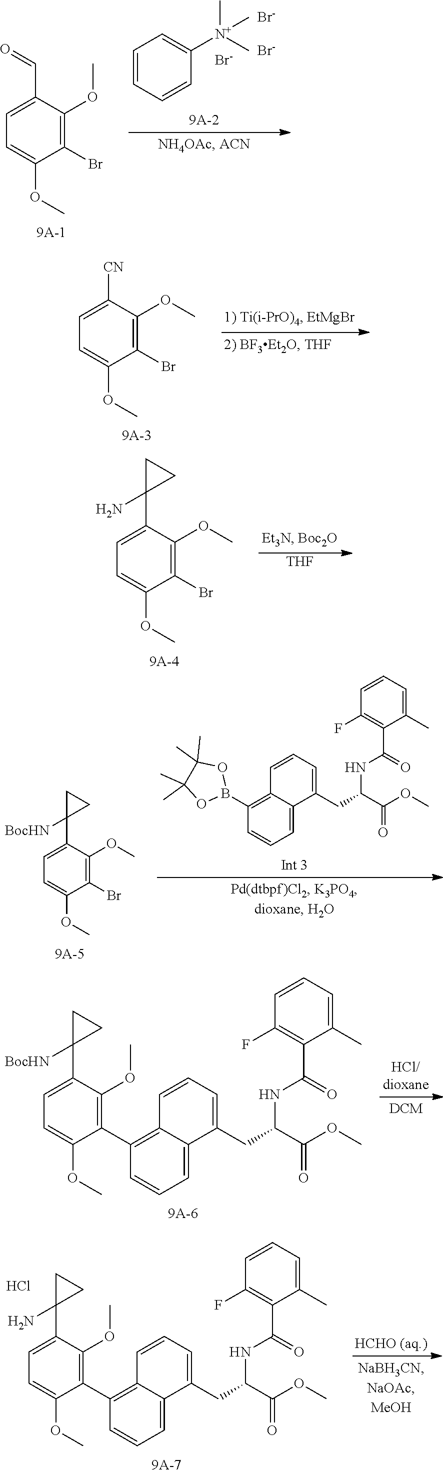

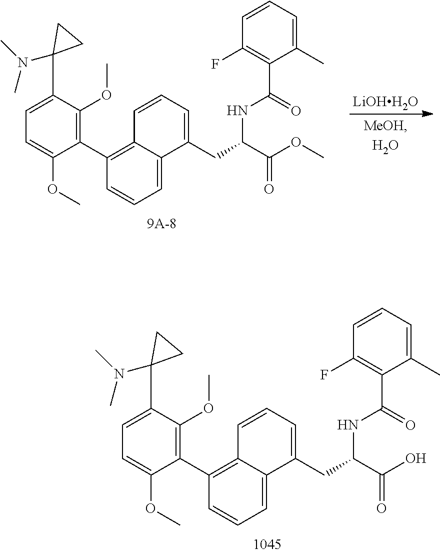

Step 1: To a solution of compound 9A-1 (2.00 g, 8.16 mmol, 1.00 eq) in ACN (200 mL) was added NH₄OAc (6.29 g, 81.6 mmol, 10.0 eq), compound 9A-2 (6.14 g, 16.3 mmol, 2.00 eq) at 25° C. and stirred at 60° C. for 12 hrs. The reaction mixture was concentrated under reduced pressure to remove ACN. The residue was diluted with H₂O 30.0 mL and extracted with ethyl acetate (30.0 mL*2). The combined organic layers were washed with brine (50.0 mL*2), concentrated under reduced pressure to give a residue. The residue was purified by column chromatography (SiO₂, TLC (Petroleum ether/Ethyl acetate=5/1) Petroleum ether/Ethyl acetate=60/1 to 15/1). Compound 9A-3 (1.70 g, 7.02 mmol, 86.0% yield) was obtained as a white solid. LC-MS: (M+H)⁺: 244.8. ¹H NMR: (400 MHz, DMSO-d₆). δ 7.84 (d, J=8.8 Hz, 1H), 7.07 (d, J=8.8 Hz, 1H), 3.95 (d, J=2.0 Hz, 6H).

Step 2: To a THF (10.0 mL) suspension of compound 9A-3 (500 mg, 2.07 mmol, 1.00 eq), Ti(i-PrO)₄ (704 mg, 2.48 mmol, 731 μL, 1.20 eq), EtMgBr (3 M, 1.51 mL, 2.20 eq) was added dropwise at −78° C., and the reaction solution was stirred at 10° C. for 0.5 hr. To the reaction solution BF₃·Et₂O (1.25 g, 4.13 mmol, 1.08 mL, 47.0% purity, 2.00 eq) was added, and the mixture was further stirred at 10° C. for 0.5 hr. The reaction mixture was quenched by addition NaOH (1 M, 20.0 ml) at 0° C. then used to the next step. Compound 9A-4 (500 mg, crude) was obtained as a yellow oil in THF. LC-MS: (M+H)⁺: 274.0.

Step 3: To a solution of compound 9A-4 (500 mg, 1.84 mmol, 1.00 eq) in THF (10.0 mL) was added Boc₂O (4.01 g, 18.3 mmol, 4.22 mL, 10.0 eq), Et₃N (3.72 g, 36.7 mmol, 5.11 mL, 20.0 eq) at 0° C. and stirred at 25° C. for 8 hrs. The reaction mixture was diluted with H₂O 10.0 mL and extracted with ethyl acetate (10.0 mL*3). The combined organic layers were washed with brine (20.0 mL*2), dried over Na₂SO₄, filtered, and concentrated under reduced pressure to give a residue. The residue was purified by Prep-HPLC (column: Phenomenex luna C 18 150*25 mm*10 μm; mobile phase: [Water (FA)-ACN]; gradient: 45%-75% B over 10 min). Compound 9A-5 (120 mg, 322 μmol, 17.5% yield) was obtained as a white solid. LC-MS: (M+H)⁺: 317.9. ¹H NMR: (400 MHz, DMSO-d₆) δ 7.41 (s, 1H), 6.79 (d, J=8.8 Hz, 1H), 3.85 (s, 3H), 3.81 (s, 3H), 1.30 (s, 9H), 1.03-0.99 (m, 2H), 0.98-0.94 (m, 2H).

Step 4: A mixture of compound 9A-5 (100 mg, 268 μmol, 1.00 eq), compound Int3 (132 mg, 268 μmol, 1.00 eq), Pd(dtbpf)Cl₂ (17.5 mg, 26.8 μmol, 0.100 eq), K₃PO₄ (171 mg, 805 μmol, 3.00 eq) in dioxane (1.00 mL), H₂O (0.200 mL) was degassed and purged with N₂ for 3 times, and then the mixture was stirred at 25° C. for 1 hr under N₂ atmosphere. The reaction mixture was diluted with H₂O 5.00 mL and extracted with ethyl acetate (10.0 mL*3). The combined organic layers were washed with brine (15.0 mL*1), concentrated under reduced pressure to give a residue. The residue was purified by Prep-TLC (SiO₂, Petroleum ether/Ethyl acetate=3/1). Compound 9A-6 (120 mg, 182 μmol, 68.0% yield) was obtained as a yellow solid. LC-MS: (M-55)⁺: 601.3. ¹H NMR: (400 MHz, DMSO-d₆). δ 8.14-8.11 (m, 1H), 7.71-7.60 (m, 1H), 7.51-7.49 (m, 1H), 7.44-7.34 (m, 2H), 7.33-7.20 (m, 4H), 7.02 (t, J=8.4 Hz, 2H), 6.83 (dd, J₁=8.8 Hz, J₂=2.8 Hz, 1H), 4.88-4.84 (m, 1H), 3.77-3.66 (m, 4H), 3.54 (d, J=5.6 Hz, 3H), 3.43 (s, 4H), 3.10 (s, 3H), 1.33 (s, 9H), 1.05-0.96 (m, 4H).

Step 5: To a solution of compound 9A-6 (120 mg, 182 μmol, 1.00 eq) in DCM (2.00 mL) was added HCl/dioxane (4 M, 1.00 mL, 21.8 eq). The mixture was stirred at 25° C. for 0.5 hr. The reaction mixture was concentrated under reduced pressure to give a residue. Compound 9A-7 (100 mg, crude, HCl) was obtained as a white solid. LC-MS: (M+23)⁺: 579.1.

Step 6: To a solution of compound 9A-7 (100 mg, 168 μmol, 1.00 eq, HCl), HCHO (109 mg 1.38 mmol, 0.100 mL, 38.0% purity, 8.18 eq), NaOAc (13.8 mg, 168 μmol, 1.00 eq) in MeOH (2.00 mL) was stirred at 25° C. for 0.2 hr, then NaBH₃CN (12.7 mg, 202 μmol, 1.20 eq) was added and stirred for 0.5 hr. The reaction mixture was diluted with H₂O 10.0 mL and extracted with DCM (20.0 mL*2). The combined organic layers were washed with brine (20.0 mL*1), concentrated under reduced pressure to give a residue. The residue was purified by Prep-TLC (SiO₂, Petroleum ether/Ethyl acetate=1/1). Compound 9A-8 (60.0 mg, crude) was obtained as a yellow solid. LC-MS: (M+H)⁺: 585.4.

Step 7: To a solution of compound 9A-8 (50.0 mg, 85.5 μmol, 1.00 eq) in MeOH (0.100 mL) was added LiOH·H₂O (3.59 mg, 85.5 μmol, 1.00 eq), H₂O (0.100 mL). The mixture was stirred at 25° C. for 0.5 hr. The reaction mixture was filtered to give a residue. The residue was purified by Prep-HPLC (column: Waters xbridge 150*25 mm 10 μm; mobile phase: [water (NH₄HCO₃)-ACN]; gradient: 15%-45% B over 14 min). Compound 1045 (24.86 mg, 43.3 μmol, 50.6% yield, 99.4% purity) was obtained as a white solid. LC-MS: (M+H)⁺: 571.1. ¹H NMR: (400 MHz, MeOD). δ 8.45 (d, J=7.2 Hz, 1H), 7.63 (t, J=7.6 Hz, 1H), 7.53 (d, J=8.8 Hz, 1H), 7.44 (t, J=6.8 Hz, 1H), 7.39 (d, J=8.0 Hz, 2H), 7.32-7.20 (m, 2H), 7.03-6.86 (m, 3H), 5.08-5.05 (m, 1H), 3.95-3.79 (m, 1H), 3.64 (d, J=3.6 Hz, 3H), 3.52-3.37 (m, 1H), 2.99 (d, J=11.6 Hz, 3H), 2.66 (d, J=7.2 Hz, 6H), 2.15-2.01 (m, 3H), 1.36-1.29 (m, 2H), 1.25-1.07 (m, 2H)

### Example 61: Synthesis of Compounds 462 and 463

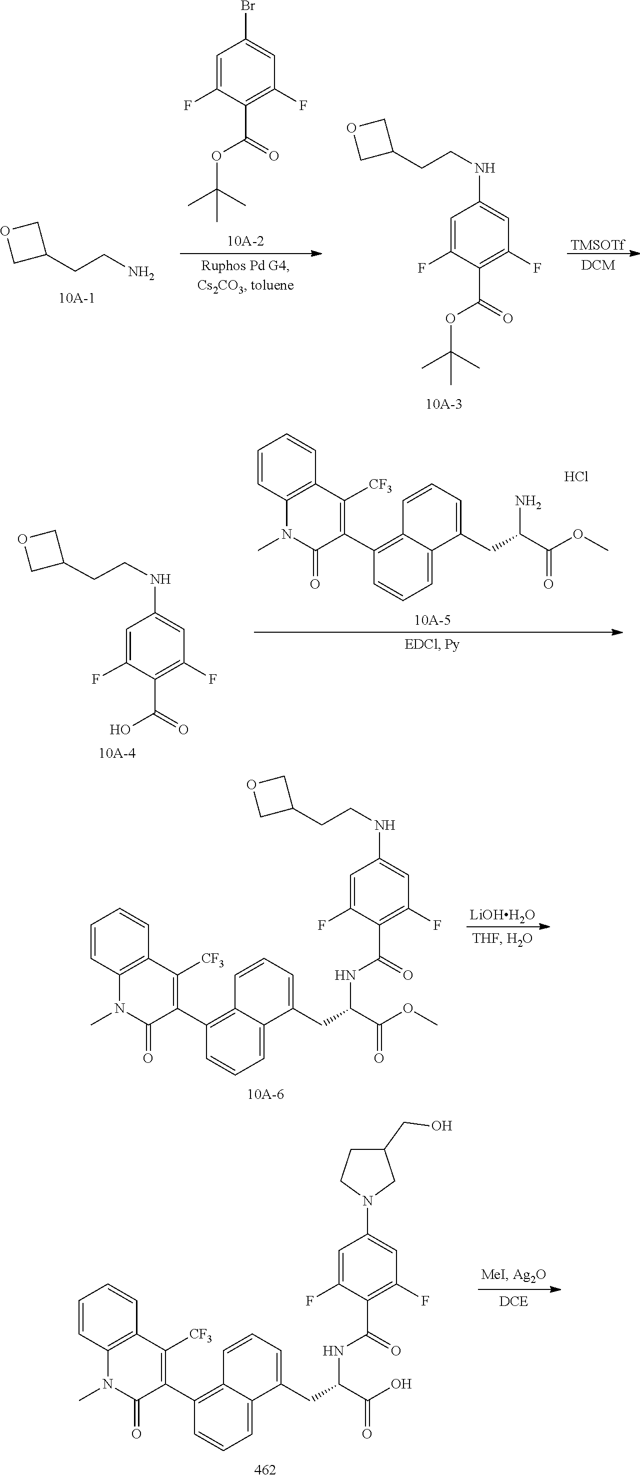

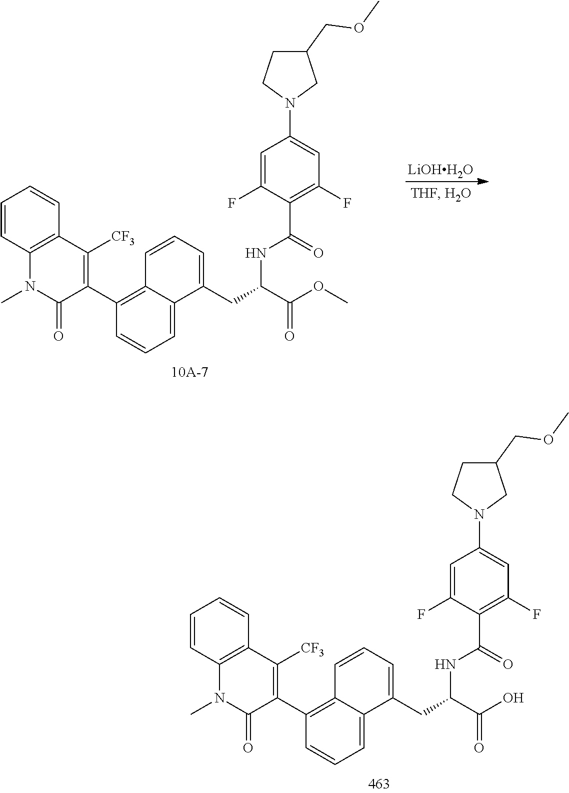 [SMILES: COCC1CCN(c2cc(F)c(C(=O)N[C@@H](Cc3cccc4c(-c5c(C(F)(F)F)c6ccccc6n(C)c5=O)cccc34)C(=O)OC)c(F)c2)C1 ; "LiOH·H2O" ; COCC1CCN(c2cc(F)c(C(=O)N[C@@H](Cc3cccc4c(-c5c(C(F)(F)F)c6ccccc6n(C)c5=O)cccc34)C(=O)O)c(F)c2)C1]

Step 1: A mixture of compound 10A-1 (300 mg, 2.97 mmol, 1.00 eq), compound 10A-2 (1.30 g, 4.45 mmol, 1.50 eq), Ruphos Pd G4 (126 mg, 148 μmol, 0.0500 eq) and Cs₂CO₃ (1.93 g, 5.93 mmol, 2.00 eq) in toluene (10.0 mL) was degassed and purged with N₂ for 3 times, and then the mixture was stirred at 90° C. for 12 hrs under N₂ atmosphere. The reaction mixture was concentrated under reduced pressure to remove toluene, and then diluted with H₂O 50.0 mL and extracted with ethyl acetate 150 mL (50.0 mL*3). The combined organic layers were washed with brine 50.0 mL (50.0 mL*1), dried over Na₂SO₄, filtered, and concentrated under reduced pressure to give a residue. The residue was purified by Prep-TLC (SiO₂, Petroleum ether:Ethyl acetate=1:1). Compound 10A-3 (3.10 mg, 854 μmol, 28.8% yield, 86.3% purity) was obtained as a yellow solid. LC-MS: (M+H)⁺: 314.1. ¹H NMR: (400 MHz, CDCl₃) δ 6.04 (d, J=11.2 Hz, 2H), 4.87-4.79 (m, 2H), 4.42 (t, J=6.0 Hz, 2H), 3.15-3.04 (m, 3H), 2.08-1.98 (m, 2H), 1.57 (s, 9H)

Step 2: To a solution of compound 10A-3 (200 mg, 551 μmol, 86.3% purity, 1.00 eq) in DCM (4.00 mL) was added TMSOTf (245 mg, 1.10 mmol, 199 μL, 2.00 eq) at 0° C., the mixture was stirred at 0° C. for 2 hrs. The reaction mixture was diluted with water 50 mL and extracted with DCM 210 mL (70.0 mL*3). The combined organic layers were washed with brine 50.0 mL (50.0 mL*1), dried over Na₂SO₄, filtered and concentrated under reduced pressure to give a residue. The crude product was used next step directly without any purification. Compound 10A-4 (140 mg, 257 μmol, 46.7% yield, 47.3% purity) was obtained as yellow oil. LC-MS: (M+H)⁺: 258.1. ¹H NMR: (400 MHz, CDCl₃) δ 6.08 (d, J=12.4 Hz, 2H), 4.48-4.38 (m, 1H), 3.84 (d, J=4.8 Hz, 2H), 3.71-3.65 (m, 2H), 2.95-2.92 (m, 1H), 2.57-2.48 (m, 2H), 2.24-2.17 (m, 2H)

Step 3: To a solution of compound 10A-4 (120 mg, 221 μmol, 47.3% purity, 1.00 eq) and compound 10A-5 (108 mg, 221 μmol, 1.00 eq, HCl) in Py (2.00 mL) was added EDCI (84.6 mg, 441 μmol, 2.00 eq), the mixture was stirred at 20° C. for 1 hrs. The reaction mixture was concentrated under reduced pressure to remove Py, then diluted with saturated NH₄Cl solution 30.0 mL and extracted with ethyl acetate 90.0 mL (30.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Dichloromethane:Methanol=10:1). Compound 10A-6 (80 mg, 115 μmol, 52.3% yield) was obtained as yellow oil. LC-MS: (M+H)⁺: 694.2. ¹H NMR: (400 MHz, CDCl₃) δ 8.27 (d, J=8.8 Hz, 1H), 8.05 (br d, J=8.0 Hz, 1H), 7.74-7.70 (m, 1H), 7.62-7.53 (m, 3H), 7.43-7.40 (m, 2H), 7.36-7.33 (m, 2H), 6.78-6.70 (m, 1H), 6.05 (d, J=12.4 Hz, 2H), 5.25-5.14 (m, 1H), 3.82 (s, 3H), 3.78-3.67 (m, 5H), 3.56 (d, J=2.8 Hz, 3H), 3.47-3.29 (m, 3H), 3.17-3.10 (m, 1H), 2.65-2.55 (m, 1H), 2.24-2.13 (m, 1H), 1.92-1.85 (m, 1H)

Step 4: To a solution of compound 10A-6 (70.0 mg, 101 μmol, 1.00 eq) in THF (2.00 mL) was added a solution of LiOH·H₂O (5.08 mg, 121 μmol, 1.20 eq) in H₂O (0.500 mL), the mixture was stirred at 20° C. for 1 hr. The reaction mixture was concentrated under reduced pressure to give a residue. The residue was purified by Prep-HPLC (column: Welch Xtimate C18 150*25 mm*5 μm; mobile phase. [water (NH₃H₂O)-ACN]; B %: 10%-40%, 8 min). Compound 462 (2.63 mg, 69.2 μmol, 68.6% yield, 94.1% purity) was obtained as white solid. LC-MS: (M+H)⁺: 680.1. ¹H NMR: (400 MHz, DMSO-d₆) δ 8.31 (t, J=9.2 Hz, 1H), 8.18 (brs, 1H), 7.93 (d, J=8.4 Hz, 1H), 7.85-7.75 (m, 2H), 7.61-7.54 (m, 1H), 7.51-7.40 (m, 2H), 7.36-7.23 (m, 3H), 6.14 (d, J=12.4 Hz, 2H), 4.72 (s, 1H), 4.52 (s, 1H), 3.72 (s, 3H), 3.70-3.61 (m, 1H), 3.48-3.40 (m, 3H), 3.25-3.16 (m, 2H), 3.06-2.97 (m, 1H), 2.46-2.37 (m, 1H), 2.08-1.95 (m, 1H), 1.83-1.67 (m, 1H)

Step 5: To a solution of Compound 462 (47.0 mg, 65.1 μmol, 94.1% purity, 1.00 eq) in DCE (2.00 mL) was added MeI (46.2 mg, 325 μmol, 20.3 μL, 5.00 eq) and Ag₂O (30.2 mg, 130 μmol, 2.00 eq), the mixture was stirred at 20° C. for 2 hrs. The reaction mixture was filtered and concentrated under reduced pressure to give a residue. The residue was purified by Prep-TLC. Compound 10A-7 (20.0 mg, 28.3 μmol, 43.4% yield) was obtained as yellow oil. LC-MS: (M+H)⁺: 708.2. ¹H NMR: (400 MHz, CDCl₃) δ 8.26 (d, J=8.8 Hz, 1H), 8.05 (brd, J=8.0 Hz, 1H), 7.72 (t, J=7.6 Hz, 1H), 7.63-7.51 (m, 2H), 7.45-7.28 (m, 5H), 6.79-6.71 (m, 1H), 6.02 (d, J=12.4 Hz, 2H), 5.23-5.14 (m, 1H), 3.86-3.77 (m, 5H), 3.76-3.64 (m, 4H), 3.61-3.52 (m, 3H), 3.46-3.24 (m, 4H), 3.15-3.08 (m, 1H), 2.61-2.54 (m, 1H), 2.18 (br s, 1H), 1.89-1.83 (m, 1H)

Step 6: To a solution of compound 10A-7 (20.0 mg, 28.3 μmol, 1.00 eq) in THF (1.00 mL) was added a solution of LiOH·H₂O (2.00 mg, 47.7 μmol, 1.69 eq) in H₂O (0.200 mL), the mixture was stirred at 20° C. for 1 hr. The residue was concentrated under reduced pressure to give a residue. The residue was purified by Prep-HPLC (column: Waters xbridge 150*25 mm 10 μm; mobile phase: [water (NH₄HCO₃)-ACN]; B %: 21%-51%, 11 min). Compound 463 (18.62 mg, 25.3 μmol, 89.7% yield, 94.4% purity) was obtained as a white solid. LC-MS: (M+H)⁺: 694.1. ¹H NMR: (400 MHz, DMSO-d₆) δ 8.37 (br s, 1H), 8.27 (t, J=8.0 Hz, 1H), 7.93 (d, J=7.2 Hz, 1H), 7.84-7.74 (m, 2H), 7.64-7.56 (m, 1H), 7.51-7.41 (m, 2H), 7.39-7.26 (m, 3H), 6.16 (d, J=11.2 Hz, 2H), 4.61-4.50 (m, 1H), 3.74-3.63 (m, 4H), 3.46-3.40 (m, 3H), 3.26 (s, 3H), 3.24-3.17 (m, 3H), 3.02-2.94 (m, 1H), 2.59-2.54 (m, 1H), 2.10-1.98 (m, 1H), 1.79-1.66 (m, 1H)

The compounds set forth in Table 27, were prepared using the synthetic procedures of Example 61, or analogous procedures as provided herein.

TABLE 27
Structure and Spectroscopic data for Compounds 1054 and 1055

| No. | Structure |
|---|---|
| 1054 | 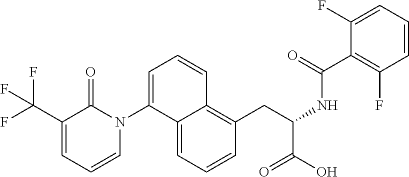 [SMILES: O=C(N[C@@H](Cc1cccc2c(-n3cccc(C(F)(F)F)c3=O)cccc12)C(=O)O)c1c(F)cccc1F] |
| | m/z = 517.3 |
| 1055 | 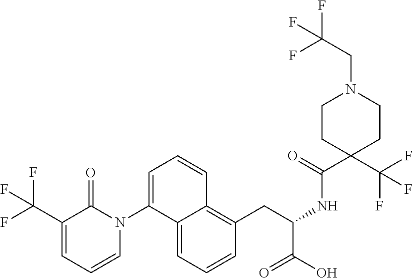 [SMILES: O=C(O)[C@H](Cc1cccc2c(-n3cccc(C(F)(F)F)c3=O)cccc12)NC(=O)C1(C(F)(F)F)CCN(CC(F)(F)F)CC1] |
| | m/z = 638.2 |

### Example 62: Synthesis of Compound 469

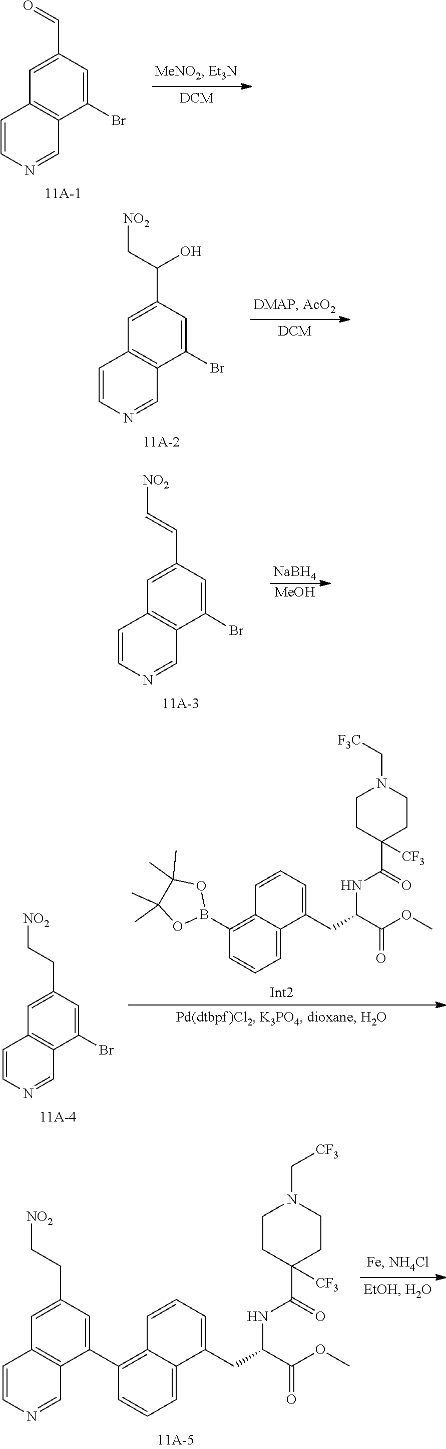

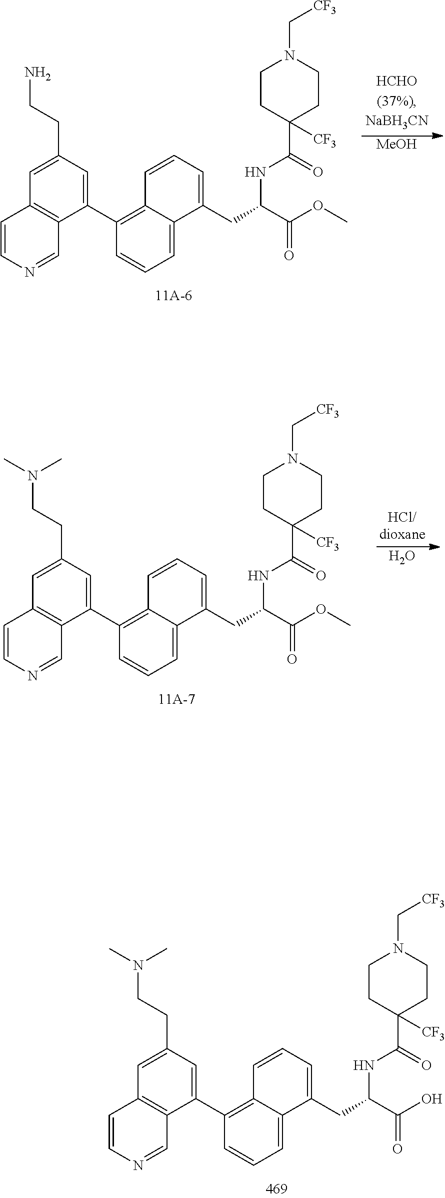 [SMILES: "MeOH" ; COC(=O)[C@H](Cc1cccc2c(-c3cc(CCN)cc4ccncc34)cccc12)NC(=O)C1(C(F)(F)F)CCN(CC(F)(F)F)CC1 ; COC(=O)[C@H](Cc1cccc2c(-c3cc(CCN(C)C)cc4ccncc34)cccc12)NC(=O)C1(C(F)(F)F)CCN(CC(F)(F)F)CC1 ; O ; CN(C)CCc1cc(-c2cccc3c(C[C@H](NC(=O)C4(C(F)(F)F)CCN(CC(F)(F)F)CC4)C(=O)O)cccc23)c2cnccc2c1]

Step 1: To a solution of compound 11A-1 (0.500 g, 2.12 mmol, 1.00 eq) and Et₃N (429 mg, 4.24 mmol, 590 μL, 2.00 eq) in DCM (10.0 mL) was added MeNO₂ (1.29 g, 21.2 mmol, 1.15 mL, 10.0 eq) at 0° C., then the mixture was stirred at 25° C. for 12 hrs. The reaction mixture was quenched by addition ice water 50.0 mL at 0° C., and then adjusted pH to 6 and extracted with ethyl acetate (40.0 mL*3). The combined organic layers were washed with brine (30.0 mL*3), dried over Na₂SO₄, filtered, and concentrated under reduced pressure to give a residue. Compound 11A-2 (0.600 g, 2.02 mmol, 95.4% yield) was obtained as yellow solid. LC-MS: (M+H)⁺: 297.0. ¹H NMR: (400 MHz, DMSO-d₆) δ 9.45 (s, 1H), 8.65 (d, J=5.6 Hz, 1H), 8.14-8.08 (m, 2H), 7.94-7.87 (m, 1H), 6.51-6.44 (m, 1H), 5.54-5.46 (m, 1H), 5.09-4.98 (m, 1H), 4.73 (dd, J₁=12.8 Hz, J₂=9.2 Hz, 1H).

Step 2: To a solution of compound 11A-2 (0.600 g, 2.02 mmol, 1.00 eq) in DCM (10.0 mL) was added DMAP (271 mg, 2.22 mmol, 1.10 eq) and Ac₂O (268 mg, 2.63 mmol, 247 μL, 1.30 eq), the mixture was stirred at 25° C. for 1 hr. The residue was diluted with H₂O (50.0 mL), brine (20.0 mL) and extracted with ethyl acetate (50.0 mL*3). The combined organic layers were washed with sat. aq. NH₄Cl (50.0 mL*3), dried over Na₂SO₄, filtered, and concentrated under reduced pressure to give a residue. Compound 11A-3 (0.530 g, 1.90 mmol, 94.0% yield) was obtained as yellow solid. LC-MS: (M+H)⁺: 281.0. ¹H NMR: (400 MHz, DMSO-d₆) δ 9.50 (s, 1H), 8.72 (d, J=5.6 Hz, 1H), 8.56-8.45 (m, 3H), 8.25 (d, J=13.2 Hz, 1H), 7.91 (d, J=5.6 Hz, 1H).

Step 3: To a solution of compound 11A-3 (500 mg, 1.79 mmol, 1.00 eq) in MeOH (5.00 ml) was added NaBH₄ (204 mg, 5.39 mmol, 3.01 eq) at 0° C., the mixture was stirred at 0° C. for 1 hr. The reaction mixture was quenched by sat. aq. NH₄Cl (20.0 mL) mL at 0° C., diluted with H₂O (20.0 mL) and extracted with ethyl acetate (20.0 mL*3). The combined organic layers were washed with brine (20.0 mL*3), dried over Na₂SO₄, filtered, and concentrated under reduced pressure to give a residue. The residue was purified by prep-TLC (SiO₂, Petroleum ether/Ethyl acetate=1/1). Compound 11A-4 (0.300 g, 1.07 mmol, 59.6% yield) was obtained as yellow solid. LC-MS: (M+H)⁻: 282.9. ¹H NMR: (400 MHz, CDCl₃) δ 9.59 (s, 1H), 8.63 (d, J=5.6 Hz, 1H), 7.74 (d, J=1.6 Hz, 1H), 7.64 (s, 1H), 7.60 (d, J=5.6 Hz, 1H), 4.75 (t, J=7.2 Hz, 2H), 3.50 (t, J=6.8 Hz, 2H).

Step 4: To solution of compound 11A-4 (100 mg, 356 μmol, 1.00 eq) and compound Int2 (263 mg, 427 μmol, 1.20 eq) in dioxane (5.00 mL) and H₂O (1.00 mL) was added Pd(dtbpf)Cl₂ (23.2 mg, 35.6 μmol, 0.100 eq) and K₃PO₄ (151 mg, 711 μmol, 2.00 eq), the mixture was stirred at 25° C. for 2 hrs. The residue was diluted with H₂O (50.0 mL) and extracted with ethyl acetate (30.0 mL*3). The combined organic layers were dried over Na₂SO₄, filtered, and concentrated under reduced pressure to give a residue. The residue was purified by prep-TLC (SiO₂, Petroleum ether/Ethyl acetate=1/1). Compound 11A-5 (0.200 g, 235 μmol, 65.9% yield, 81.0% purity) was obtained as yellow solid. LC-MS: (M+H)⁺: 691.4.

Step 5: To a solution of compound 11A-5 (170 mg, 199 μmol, 1.00 eq) in EtOH (6.00 mL) and H₂O (3.00 mL) was added NH₄Cl (107 mg, 2.00 mmol, 10.0 eq) and Fe (112 mg, 2.01 mmol, 10.1 eq) at 25° C., the mixture was stirred at 80° C. for 3 hrs. The reaction mixture was filtered, and the filter was concentrated to get s residue. The residue was diluted with sat. aq. NaHCO₃ (30.0 mL) and extracted with ethyl acetate (20.0 mL*3). The combined organic layers were washed with brine (20.0 mL*2), dried over Na₂SO₄, filtered, and concentrated under reduced pressure to give a residue. Compound 11A-6 (130 mg, 197 μmol, 98.7% yield) was obtained as yellow solid. LC-MS: (M+H)⁺: 661.3. ¹H NMR: (400 MHz, DMSO-d₆) δ 8.60 (brd, J=5.2 Hz, 1H), 8.50-8.39 (m, 2H), 8.28 (brd, J=8.8 Hz, 1H), 7.92-7.84 (m, 2H), 7.76 (brt, J=6.8 Hz, 1H), 7.58 (brd, J=6.8 Hz, 1H), 7.51 (brd, J=2.8 Hz, 1H), 7.40 (br d, J=7.2 Hz, 1H), 7.33-7.22 (m, 1H), 7.13 (brd, J=8.4 Hz, 1H), 4.95-4.78 (m, 1H), 3.92-3.79 (m, 1H), 3.78-3.52 (m, 5H), 2.99-2.88 (m, 5H), 2.81-2.65 (m, 2H), 2.35-2.10 (m, 3H), 1.97-1.84 (m, 1H), 1.62 (brt, J=12.4 Hz, 2H).

Step 6: To a solution of compound 11A-6 (110 mg, 166 μmol, 1.00 eq) in MeOH (2.00 mL) was added HCHO (54.1 mg, 666 μmol, 49.6 μL, 37.0% purity, 4.00 eq) and NaBH₃CN (20.9 mg, 333 μmol, 2.00 eq), the mixture was stirred at 25° C. for 1 hr. The residue was diluted with sat. aq. NaHCO₃ (10.0 mL) and extracted with ethyl acetate (10.0 mL*3). The combined organic layers were dried over Na₂SO₄, filtered and concentrated under reduced pressure to give a residue. Compound 11A-7 (110 mg, crude) was obtained as yellow oil. LC-MS: (M+H)⁺: 689.2.

Step 7: To a solution of compound 11A-7 (110 mg, 160 μmol, 1.00 eq) in H₂O (2.00 mL) was added HCl/dioxane (4.00 M, 9.00 mL, 225 eq), the mixture was stirred at 60° C. for 1 hr. The mixture was concentrated under reduced pressure to give a residue. The residue was purified by prep-HPLC (column: Waters xbridge 150*25 mm 10 μm; mobile phase: [water (NH₄HCO₃)-ACN]; B %: %, isocratic elution mode). Compound 469 (52.60 mg, 77.7 μmol, 48.6% yield, 99.6% purity) was obtained as white solid. LC-MS: (M+H)⁺: 675.3. ¹H NMR: (400 MHz, DMSO-d₆) δ 8.48-8.41 (m, 2H), 8.41-8.30 (m, 2H), 7.92 (s, 1H), 7.86 (dd, J₁=5.6 Hz, J₂=3.2 Hz, 1H), 7.79-7.72 (m, 1H), 7.59-7.52 (m, 2H), 7.41 (t, J=6.0 Hz, 1H), 7.28-7.22 (m, 1H), 7.09 (br d, J=8.4 Hz, 1H), 4.87-4.67 (m, 1H), 3.89-3.77 (m, 1H), 3.54-3.50 (m, 1H), 3.05-2.86 (m, 4H), 2.85-2.69 (m, 4H), 2.31-2.13 (m, 8H), 2.10-1.78 (m, 2H), 1.67-1.52 (m, 2H).

### Example 63: Synthesis of Compound 1046

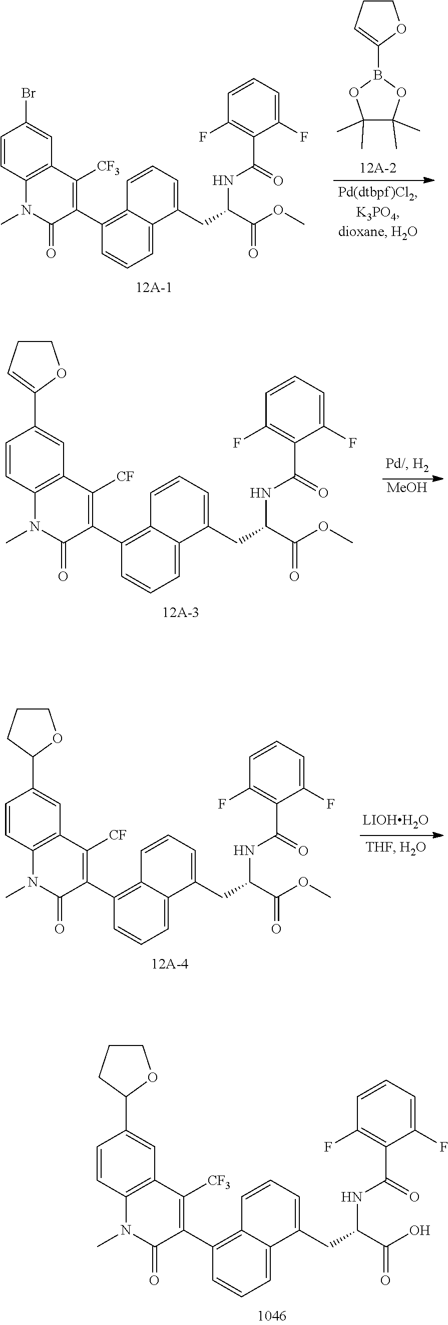

Step 1: A mixture of compound 12A-1 (200 mg, 297 μmol, 1.00 eq), compound 12A-2 (116 mg, 594 μmol, 2.00 eq), Pd(dtbpf)Cl₂ (58.1 mg, 89.1 μmol, 0.300 eq) and K₃PO₄ (189 mg, 891 μmol, 3.00 eq) in dioxane (4.00 mL), H₂O (1.00 mL) was degassed and purged with N₂ for 3 times, and then the mixture was stirred at 25° C. for 2 hrs under N₂ atmosphere. The reaction mixture was diluted with water 30.0 mL and extracted with ethyl acetate 90.0 mL (30.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Petroleum ether:Ethyl acetate=0:1). Compound 12A-3 (160 mg, 213 μmol, 71.9% yield, 88.4% purity) was obtained as yellow oil. LC-MS: (M+H)⁺: 663.3. ¹H NMR: (400 MHz, CDCl₃) $8.68 (br s, 1H), 8.38-8.20 (dd, J₁=25.2 Hz, J₂=8.8 Hz, 2H), 7.67-7.54 (m, 2H), 7.43-7.33 (m, 4H), 7.31-7.28 (m, 1H), 6.95 (t, J=8.0 Hz, 2H), 6.71-6.60 (m, 1H), 5.32-5.18 (m, 1H), 3.91-3.82 (m, 4H), 3.80-3.79 (m, 1H), 3.60 (d, J=5.6 Hz, 3H), 3.22 (t, J=6.8 Hz, 2H), 2.14-2.07 (m, 2H)

Step 2: To a solution of compound 12A-3 (80.0 mg, 107 μmol, 88.4% purity, 1.00 eq) in MeOH (3.00 mL) was added Pd/C (20.0 mg, 10.0% purity) under N₂. The suspension was degassed under vacuum and purged with H₂ several times. The mixture was stirred under H₂ (15 psi) at 25° C. for 10 hrs. The residue was purified by Prep-HPLC (column: Waters xbridge 150*25 mm 10 μm; mobile phase: [water (NH₄HCO₃)-ACN]; B %: 50%-80%, 10 min). Compound 12A-4 (45.0 mg, 67.7 μmol, 63.4% yield, 100% purity) was obtained as yellow oil. LC-MS: (M+H)⁺: 665.4. ¹H NMR: (400 MHz, CDCl₃) δ 8.23 (d, J=8.8 Hz, 1H), 7.97 (br s, 1H), 7.71 (d, J=8.8 Hz, 1H), 7.60 (t, J=7.2 Hz, 1H), 7.51 (d, J=9.2 Hz, 1H), 7.46-7.41 (m, 1H), 7.40-7.32 (m, 3H), 7.31-7.28 (m, 1H), 6.95 (t, J=8.4 Hz, 2H), 6.67 (dd, J₁=15.2 Hz, J₂=7.2 Hz, 1H), 5.30-5.20 (m, 1H), 5.03 (t, J=7.6 Hz, 1H), 4.21-4.10 (m, 1H), 4.05-3.96 (m, 1H), 3.88-3.79 (m, 4H), 3.79-3.68 (m, 1H), 3.59 (d, J=2.4 Hz, 3H), 2.49-2.38 (m, 1H), 2.14-2.03 (m, 2H), 1.92-1.81 (m, 1H)

Step 3: To a solution of compound 12A-4 (45.0 mg, 67.7 μmol, 100% purity, 1.00 eq) in THF (1.00 mL) was added a solution of LiOH·H₂O (3.41 mg, 81.2 μmol, 1.20 eq) in H₂O (0.500 mL), the mixture was stirred at 25° C. for 1 hr. The reaction mixture was concentrated under reduced pressure to give a residue. The residue was purified by Prep-HPLC (column: Waters xbridge 150*25 mm 10 μm; mobile phase: [water (NH₄HCO₃)-ACN]; B %: 22%-52%, 10 min). Compound 1046 (12.29 mg, 18.6 μmol, 27.5% yield, 98.5% purity) was obtained as a white solid. LC-MS: (M+H)⁺: 651.3. ¹H NMR: (400 MHz, DMSO-d₆) δ 9.05 (brs, 1H), 8.25 (d, J=7.2 Hz, 1H), 7.86 (br s, 1H), 7.75 (s, 2H), 7.66-7.59 (m, 1H), 7.51-7.42 (m, 2H), 7.41-7.37 (m, 1H), 7.36-7.29 (m, 2H), 7.12 (t, J=8.0 Hz, 2H), 4.98 (t, J=7.2 Hz, 1H), 4.75-4.61 (m, 1H), 4.09-3.99 (m, 1H), 3.93-3.82 (m, 1H), 3.78-3.68 (m, 4H), 3.42-3.39 (m, 1H), 2.44-2.35 (m, 1H), 2.03-1.92 (m, 2H), 1.77-1.63 (m, 1H)

### Example 64: General Scheme 13

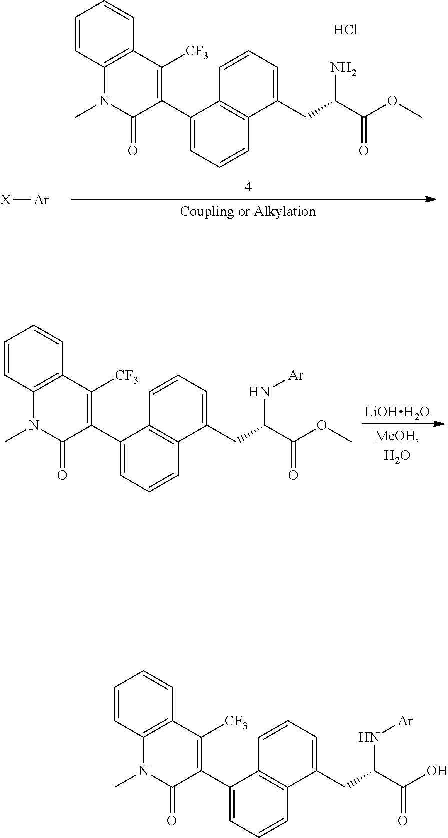

### Synthesis of Compound 326

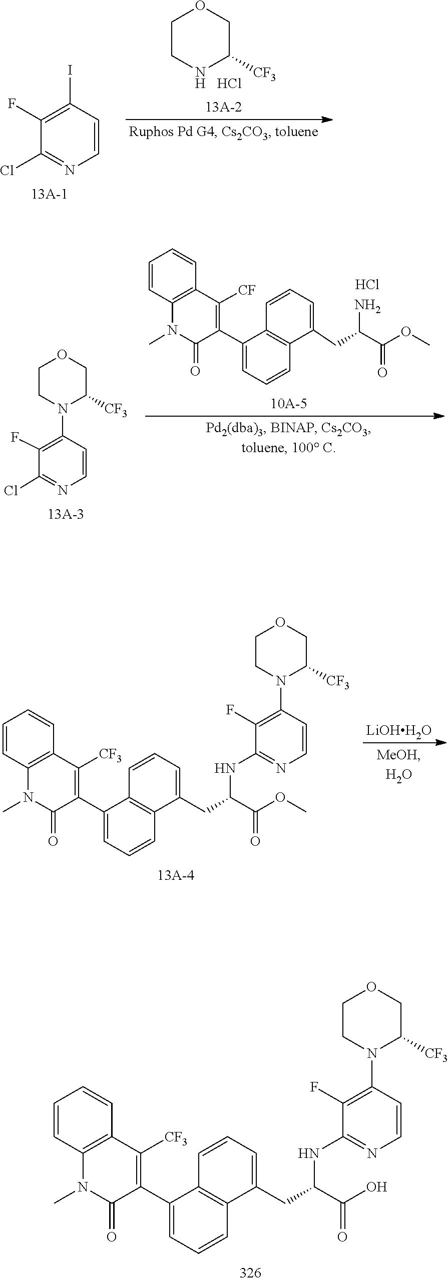

Step 1: A mixture of compound 13A-1 (500 mg, 1.94 mmol, 1.00 eq), compound 13A-2 (409 mg, 2.14 mmol, 1.10 eq, HCl), Ruphos Pd G4 (165 mg, 194 μmol, 0.100 eq) and Cs₂CO₃ (1.90 g, 5.83 mmol, 3.00 eq) in toluene (10.0 mL) was degassed and purged with N₂ for 3 times, and then the mixture was stirred at 90° C. for 12 hrs under N₂ atmosphere. The residue was diluted with H₂O (50.0 mL) and extracted with ethyl acetate (30.0 mL*3). The combined organic layers were washed with brine (30.0 mL*3), dried over Na₂SO₄, filtered, and concentrated under reduced pressure to give a residue. The residue was purified by column chromatography (SiO₂, Petroleum ether/Ethyl acetate=1/0 to 1/1). Compound 13A-3 (50.0 mg, 176 μmol, 9.04% yield) was obtained as yellow oil.

Step 2: A mixture of compound 13A-3 (30.0 mg, 105 μmol, 1.00 eq), compound 13A-4 (51.7 mg, 105 μmol, 1.00 eq, HCl), Pd₂(dba)₃ (9.65 mg, 10.5 μmol, 0.100 eq), BINAP (6.56 mg, 10.5 μmol, 0.100 eq) and Cs₂CO₃ (103 mg, 316 μmol, 3.00 eq) in toluene (2.00 mL) was degassed and purged with N₂ for 3 times, and then the mixture was stirred at 100° C. for 12 hrs under N₂ atmosphere. The mixture was concentrated under reduced pressure to give a residue. The residue was purified by prep-TLC (SiO₂, Petroleum ether/Ethyl acetate=1/1). Compound 13A-5 (6.00 mg, 6.92 μmol, 6.56% yield, 81.0% purity) was obtained as yellow oil. LC-MS: (M+H)⁺: 702.9.

Step 3: To a solution of compound 13A-5 (6.00 mg, 6.92 μmol, 81.0% purity, 1.00 eq) in MeOH (0.500 mL) was added a solution of LiOH H₂O (581 ug, 13.8 μmol, 2.00 eq) in H₂O (0.500 mL), then the mixture was stirred at 20° C. for 1 hr. The mixture was concentrated under reduced pressure to give a residue. The residue was purified by prep-HPLC (column: Phenomenex C18 150*25 mm*10 μm; mobile phase: [water (NH₄HCO₃)-ACN]; B %: 25%-55%, 8 min). Compound 326 (4.73 mg, 6.51 μmol, 94.1% yield, 94.8% purity) was obtained as white solid. LC-MS: (M+H)⁺: 689.1. ¹H NMR: (400 MHz, CD₃CN) δ 8.35-8.22 (m, 1H), 8.07-7.94 (m, 1H), 7.76 (t, J=8.0 Hz, 1H), 7.69-7.56 (m, 3H), 7.50-7.37 (m, 3H), 7.35-7.25 (m, 2H), 6.42-6.32 (m, 1H), 5.55-5.47 (m, 1H), 4.95-4.82 (m, 1H), 4.34-4.24 (m, 1H), 4.14 (brd, J=12.8 Hz, 1H), 3.98-3.79 (m, 3H), 3.72 (s, 3H), 3.68-3.51 (m, 3H), 3.20-3.10 (m, 1H).

### Example 65: Synthesis of Compound 1047

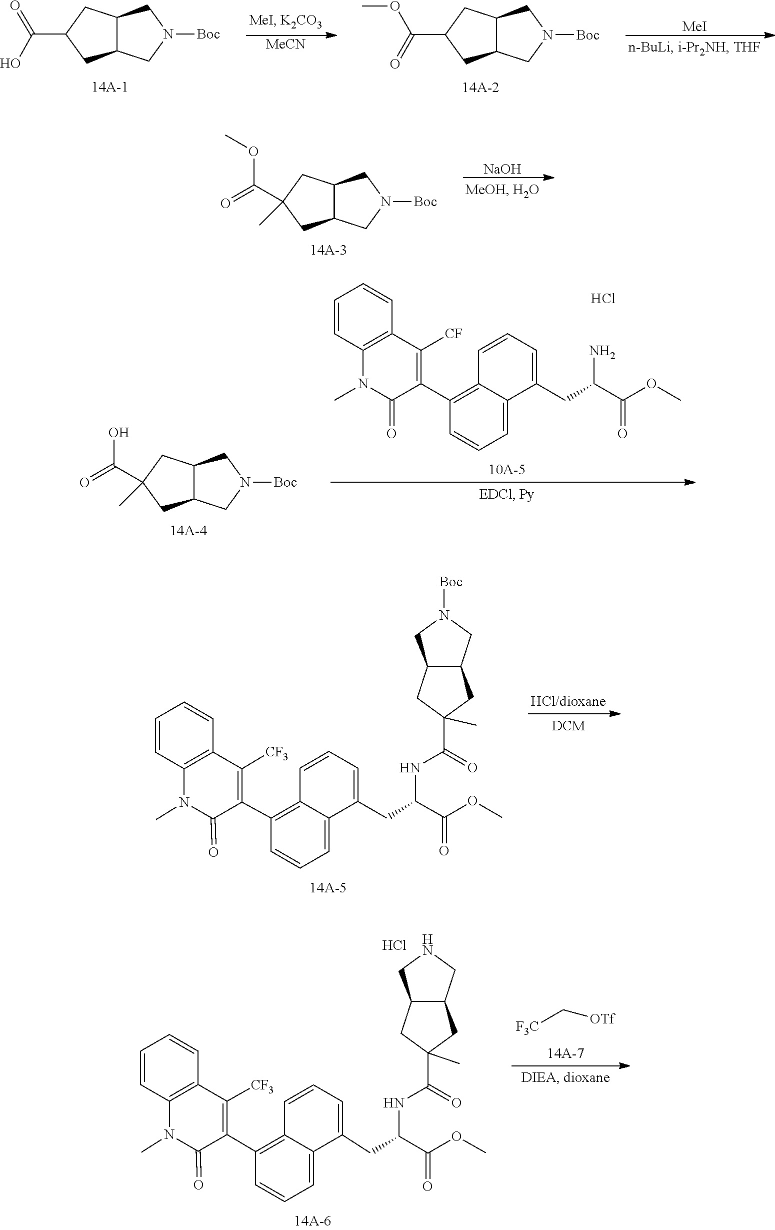

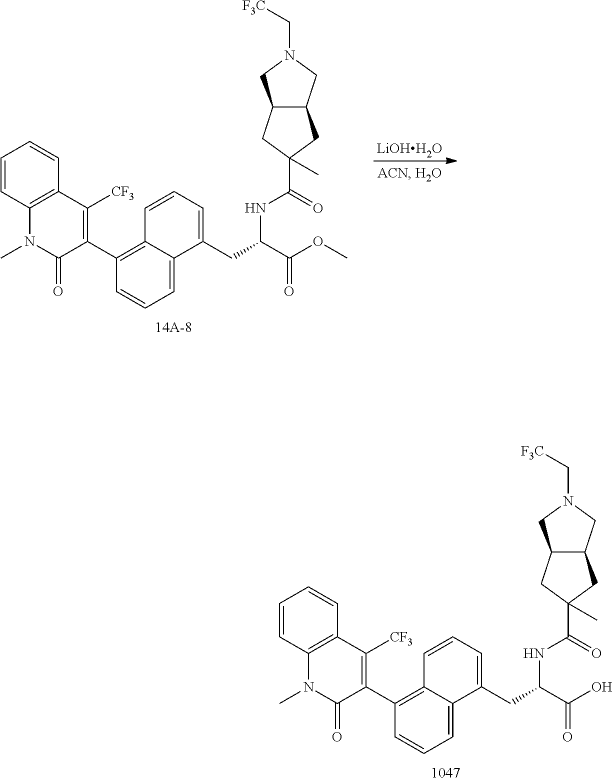

Step 1: To a solution of compound 14A-1 (350 mg, 1.37 mmol, 1.00 eq) in MeCN (5.00 mL) was added MeI (584 mg, 4.11 mmol, 256 μL, 3.00 eq) and K₂CO₃ (568 mg, 4.11 mmol, 3.00 eq), the mixture was stirred at 20° C. for 3 hrs. The reaction mixture was concentrated under reduced pressure to remove MeCN, then diluted with water 30.0 mL and extracted with ethyl acetate 90.0 mL (30.0 mL*3). The combined organic layers were washed with brine 30.0 mL (30.0 mL*1), dried over Na₂SO₄, filtered, and concentrated under reduced pressure to give a residue. The residue was purified by column chromatography (SiO₂, Petroleum ether:Ethyl acetate=1:0 to 8:1). Compound 14A-2 (360 mg, 1.34 mmol, 97.5% yield) was obtained as yellow oil. ¹H NMR: (400 MHz, CDCl₃) δ 3.69 (s, 3H), 3.53-3.42 (m, 2H), 3.27 (dd, J₁=11.2 Hz, J₂=3.2 Hz, 2H), 2.92-2.80 (m, 1H), 2.70-2.58 (m, 2H), 2.25-2.14 (m, 2H), 1.79-1.66 (m, 2H), 1.46 (s, 9H)

Step 2: To a solution of i-Pr₂NH (3.58 g, 35.4 mmol, 5.00 mL, 26.5 eq) in THF (10.0 mL) was added n-BuLi (2.50 M, 14.2 mL, 26.5 eq) at −40° C. under N₂ atmosphere, the mixture was stirred at −40° C. for 1 hr, then the mixture was warmed to −10° C. to get a solution (LDA, about 1.20 M, 30.0 mL). To a solution of compound 14A-2 (360 mg, 1.34 mmol, 1.00 eq) in THF (5.00 mL) was added LDA (the same as above, about 1.20 M, 5.57 mL) at −78° C. stirred for 1 hr. Mel (949 mg, 6.68 mmol, 416 μL, 5.00 eq) was added at −78° C. and the mixture was slowly warmed to 25° C. The mixture was stirred at 25° C. for 5 hrs. The reaction mixture was quenched by addition saturated NH₄Cl solution 50.0 mL at 0° C., and then extracted with ethyl acetate 150 mL (50.0 mL*3). The combined organic layers were washed with brine 50.0 mL (50.0 mL*1), dried over Na₂SO₄, filtered, and concentrated under reduced pressure to give a residue. The residue was purified by column chromatography (SiO₂, Petroleum ether:Ethyl acetate=0:1 to 5:1). Compound 14A-3 (352 mg, 1.24 mmol, 92.9% yield) was obtained as yellow oil. ¹H NMR: (400 MHz, CDCl₃) δ 3.69 (s, 3H), 3.51-3.38 (m, 2H), 3.26 (d, J=9.6 Hz, 2H), 2.81-2.68 (m, 2H), 2.04-1.93 (m, 2H), 1.90-1.77 (m, 2H), 1.46 (s, 9H), 1.25 (s, 3H)

Step 3: To a solution of compound 14A-3 (180 mg, 63.0 μmol, 1.00 eq) in MeOH (2.00 mL) was added a solution of NaOH (50.8 mg, 1.27 mmol, 2.00 eq) in H₂O (0.500 mL), the mixture was stirred at 20° C. for 12 hrs, then stirred at 60° C. for 4 hrs. The reaction mixture was concentrated under reduced pressure to remove MeOH, then diluted with water 30.0 mL and extracted with ethyl acetate 150 mL (50.0 mL*3). The combined organic layers were dried over Na₂SO₄, filtered and concentrated under reduced pressure to give a residue. The crude product was used next step directly without any purification. Compound 14A-4 (160 mg, crude) was obtained as white solid. LC-MS: (M−H)⁻: 268.0. ¹H NMR: (400 MHz, CDCl₃) δ 3.47 (br s, 2H), 3.27 (br s, 2H), 2.83-2.69 (m, 2H), 2.06-1.98 (m, 2H), 1.91-1.79 (m, 2H), 1.50-1.41 (m, 9H), 1.28 (s, 3H)

Step 4: To a solution of compound 14A-4 (100 mg, 371 μmol, 1.00 eq) and compound 10A-5 (182 mg, 371 μmol, 1.00 eq, HCl) in Py (3.00 mL) was added EDCI (142 mg, 743.7 μmol, 2.00 eq), the mixture was stirred at 20° C. for 2 hrs. The reaction mixture was concentrated under reduced pressure to remove Py, then diluted with saturated NH₄Cl solution 50.0 mL and extracted with dichloromethane 120 mL (40.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Dichloromethane:Methanol=10:1). Compound 14A-5 (242 mg, 320 μmol, 86.2% yield, 93.3% purity) was obtained as light-yellow solid. LC-MS: (M−H)⁺: 706.3. ¹H NMR: (400 MHz, CDCl₃) δ 8.16 (d, J=8.8 Hz, 1H), 8.05 (d, J=8.0 Hz, 1H), 7.73 (t, J=8.4 Hz, 1H), 7.63-7.51 (m, 2H), 7.46-7.38 (m, 2H), 7.35-7.28 (m, 2H), 7.26-7.23 (m, 1H), 6.07-5.91 (m, 1H), 5.08-4.88 (m, 1H), 3.82 (s, 3H), 3.74-3.60 (m, 5H), 3.48-3.38 (m, 2H), 3.31-3.16 (m, 2H), 2.78-2.65 (m, 2H), 1.87-1.66 (m, 4H), 1.45 (s, 9H), 1.09 (d, J=7.6 Hz, 3H)

Step 5: To a solution of compound 14A-5 (200 mg, 264 μmol, 1.00 eq) in DCM (3.00 mL) was added HCl/dioxane (4.00 M, 0.800 mL, 12.1 eq), the mixture was stirred at 20° C. for 1 hr. The reaction mixture was concentrated under reduced pressure to give a residue. Compound 14A-6 (160 mg, crude, HCl) was obtained as yellow oil. LC-MS: (M+H)⁺: 606.3

Step 6: To a solution of compound 14A-6 (140 mg, 218 μmol, 1.00 eq, HCl) and compound 14A-7 (50.6 mg, 218 μmol, 1.00 eq) in dioxane (5.00 mL) was added DIEA (84.5 mg, 654 μmol, 114 μL, 3.00 eq), the mixture was stirred at 25° C. for 5 hrs. The reaction mixture was concentrated under reduced to remove solvent, then diluted with water 50.0 mL and extracted with ethyl acetate 150 mL (50.0 mL*3). The combined organic layers were washed with brine 30.0 mL (30.0 mL*1), dried over Na₂SO₄, filtered, and concentrated under reduced pressure to give a residue. The reaction mixture was purified by Prep-TLC (SiO₂, Petroleum ether:Ethyl acetate=0:1). Compound 14A-8 (140 mg, 201 μmol, 92.3% yield, 98.8% purity) was obtained as light-yellow solid. LC-MS: (M+H)⁺: 688.4. ¹H NMR: (400 MHz, DMSO-d₆) δ 8.15 (t, J=8.4 Hz, 1H), 7.93 (d, J=7.3 Hz, 1H), 7.86-7.74 (m, 3H), 7.66-7.58 (m, 1H), 7.50-7.44 (m, 1H), 7.43-7.29 (m, 4H), 4.69-4.47 (m, 1H), 3.72 (d, J=2.4 Hz, 3H), 3.68-3.58 (m, 4H), 3.56-3.43 (m, 1H), 3.18-3.05 (m, 2H), 2.60-2.51 (m, 4H), 2.47-2.37 (m, 2H), 1.80-1.63 (m, 2H), 1.58-1.45 (m, 2H), 1.00 (d, J=4.8 Hz, 3H)

Step 7: To a solution of compound 14A-8 (120 mg, 172 μmol, 1.00 eq) in ACN (2.00 mL) was added a solution of LiOH·H₂O (8.68 mg, 207 μmol, 1.20 eq) in H₂O (0.500 mL), the mixture was stirred at 20° C. for 1 hr. The residue was purified by Prep-HPLC (column: Waters xbridge 150*25 mm 10 μm; mobile phase: [water (NH₄HCO₃)-ACN]; B %: 22%-52%, 14 min). Compound 1047 (84.87 mg, 125 μmol, 72.6% yield, 99.3% purity) was obtained as white solid. LC-MS: (M+H)⁻: 674.4. ¹H NMR: (400 MHz, DMSO-d₆) δ 8.27-8.19 (m, 1H), 7.93 (d, J=7.2 Hz, 1H), 7.84-7.74 (m, 2H), 7.62-7.53 (m, 1H), 7.50-7.24 (m, 6H), 4.54-4.35 (m, 1H), 3.74-3.61 (m, 4H), 3.46-3.41 (m, 1H), 3.16-3.07 (m, 2H), 2.59-2.52 (m, 3H), 2.46-2.37 (m, 2H), 2.32-2.07 (m, 1H), 1.79-1.41 (m, 4H), 1.08-0.92 (m, 3H)

### Example 66: Synthesis of Compound 1048

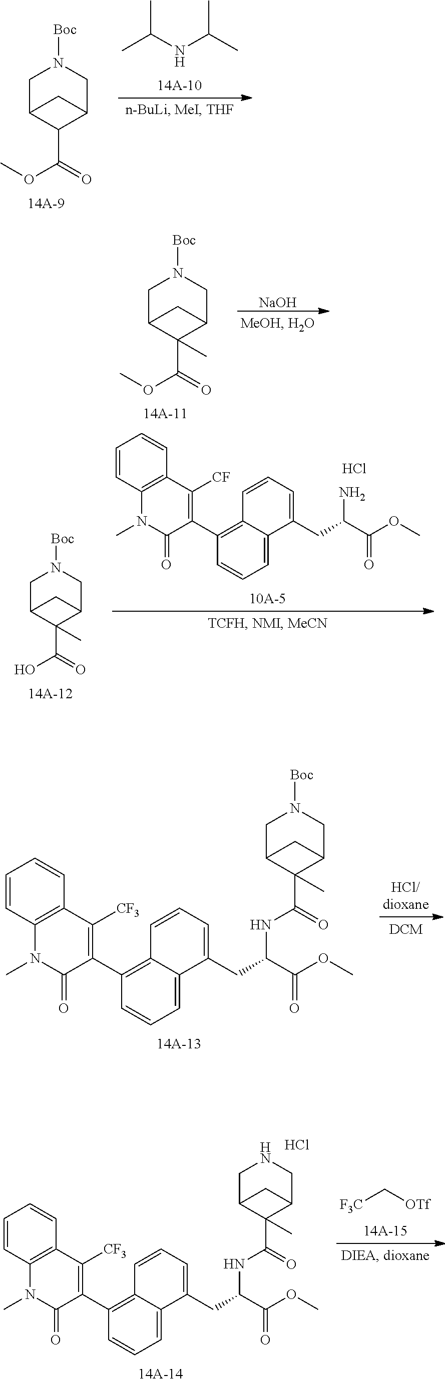

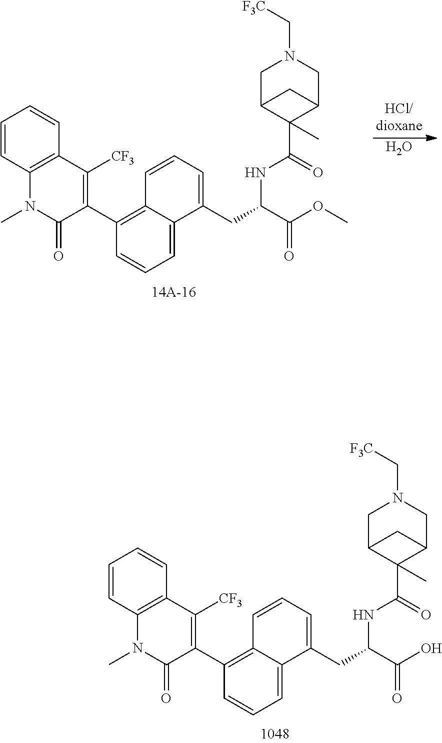 [SMILES: COC(=O)[C@H](Cc1cccc2c(-c3c(C(F)(F)F)c4ccccc4n(C)c3=O)cccc12)NC(=O)C1(C)C2CC1CN(CC(F)(F)F)C2 ; O ; Cn1c(=O)c(-c2cccc3c(C[C@H](NC(=O)C4(C)C5CC4CN(CC(F)(F)F)C5)C(=O)O)cccc23)c(C(F)(F)F)c2ccccc21]

Step 1: To a solution of compound 14A-9 (3.58 g, 35.4 mmol, 5.00 mL, 45.2 eq) in THF (10.0 mL) was added n-BuLi (2.50 M, 14.2 mL, 45.2 eq) at −40° C. under N₂, the mixture was stirred at −40° C. for 1 hr. Then the mixture was warmed to −10° C. to get a solution (LDA, about 1.2 M, 30.0 mL). To a solution of compound 14A-10 (0.200 g, 783 μmol, 1.00 eq) in THF (10.0 mL) was added LDA (the same as above, about 1.20 M, 3.26 mL) at −78° C. and stirred for 1 hr. Mel (556 mg, 3.92 mmol, 244 μL, 5.00 eq) was added at −78° C. and the mixture was slowly warmed to 25° C. The mixture was stirred at 25° C. for 12 hrs. The reaction mixture was quenched by addition sat. aq. NH₄Cl (40.0 mL) at 0° C., and then diluted with H₂O (10.0 mL) and extracted with ethyl acetate (20.0 mL*3). The combined organic layers were washed with brine (20.0 mL*3), dried over Na₂SO₄, filtered and concentrated under reduced pressure to give a residue. The residue was purified by prep-TLC (SiO₂, Petroleum ether/Ethyl acetate=5/1). Compound 14A-11 (0.156 g, 579 μmol, 73.9% yield) was obtained as yellow solid. ¹H NMR: (400 MHz, CDCl₃) δ 3.98-3.90 (m, 2H), 3.65 (s, 3H), 3.33 (d, J=11.6 Hz, 2H), 2.42-2.28 (m, 3H), 1.49 (s, 3H), 1.46 (s, 9H), 1.18 (d, J=8.4 Hz, 1H)

Step 2: To a solution of compound 14A-11 (0.156 g, 579 μmol, 1.00 eq) in MeOH (3.00 mL) was added a solution of NaOH (116 mg, 2.90 mmol, 5.00 eq) in H₂O (1.00 mL) at 25° C., then the mixture was stirred at 70° C. for 30 hrs. The reaction mixture was concentrated under reduced pressure to remove MeOH. The residue was diluted with H₂O (10.0 mL) and adjusted pH to 4 with HCl (1.00 M). The aqueous phase was extracted with ethyl acetate (20.0 mL*3). The combined organic layers were washed with brine (20.0 mL*2), dried over Na₂SO₄, filtered, and concentrated under reduced pressure to give a residue. Compound 14A-12 (0.140 g, 548 μmol, 94.7% yield) was obtained as white solid. ¹H NMR: (400 MHz, CDCl₃) δ 4.04-3.94 (m, 2H), 3.37 (br d, J=11.6 Hz, 2H), 2.44-2.28 (m, 3H), 1.54 (s, 3H), 1.45 (s, 9H), 1.19 (d, J=8.8 Hz, 1H)

Step 3: To a solution of compound 14A-12 (50.0 mg, 196 μmol, 1.00 eq) and compound 10A-5 (96.1 mg, 196 μmol, 1.00 eq, HCl) in MeCN (5.00 mL) was added TCFH (165 mg, 588 μmol, 3.00 eq) and NMI (80.4 mg, 979 μmol, 78.1 μL, 5.00 eq), the mixture was stirred at 25° C. for 2 hrs. The residue was diluted with H₂O (20.0 mL) and extracted with ethyl acetate (20.0 mL*3). The combined organic layers were washed with brine (20.0 mL*2), dried over Na 2SO₄, filtered, and concentrated under reduced pressure to give a residue. The residue was purified by prep-TLC (SiO₂, Dichloromethane/Methanol=10/1). Compound 14A-13 (0.130 g, 188 μmol, 96.0% yield) was obtained as yellow oil. LC-MS: (M-99): 592.4

Step 4: To a solution of compound 14A-13 (0.130 g, 188 μmol, 1.00 eq) in DCM (5.00 mL) was added HCl/dioxane (4.00 M, 2.00 mL, 42.6 eq), the mixture was stirred at 25° C. for 1 hr. The reaction mixture was concentrated under reduced pressure to give a residue. Compound 14A-14 (0.110 g, 175 μmol, 93.2% yield, HCl) was obtained as yellow solid. LC-MS: (M+H)⁺: 592.5

Step 5: To a solution of compound 14A-14 (90.0 mg, 143 μmol, 1.00 eq, HCl) and compound 14A-15 (66.5 mg, 287 μmol, 2.00 eq) in dioxane (5.00 mL) was added DIEA (92.6 mg, 716 μmol, 125 μL, 5.00 eq) at 25° C., the mixture was stirred at 90° C. for 3 hrs. The residue was diluted with H₂O (20.0 mL) and extracted with ethyl acetate (20.0 mL*3). The combined organic layers were washed with brine (20.0 mL*2), dried over Na₂SO₁, filtered, and concentrated under reduced pressure to give a residue. The residue was purified by prep-TLC (SiO₂, Petroleum ether/Ethyl acetate=1/1). Compound 14A-16 (73.0 mg, 108 μmol, 75.6% yield) was obtained as white solid. LC-MS: (M+H)⁺: 674.5. ¹H NMR: (400 MHz, DMSO-d₆) δ 8.17 (dd, J₁=8.4 Hz, J_{2=2.0} Hz, 1H), 7.93 (brd, J=7.2 Hz, 1H), 7.86-7.75 (m, 3H), 7.68-7.59 (m, 1H), 7.52-7.29 (m, 5H), 4.78-4.62 (m, 1H), 3.71 (d, J=2.0 Hz, 3H), 3.67-3.50 (m, 4H), 3.46-3.38 (m, 1H), 3.29-3.24 (m, 1H), 3.22-3.05 (m, 2H), 2.97-2.87 (m, 2H), 2.81-2.66 (m, 1H), 2.29-2.14 (m, 3H), 1.31-1.21 (m, 4H).

Step 6: To a solution of compound 14A-16 (63.0 mg, 93.5 μmol, 1.00 eq) in H₂O (1.00 mL) was added HCl/dioxane (4.00 M, 6.00 mL, 257 eq) at 25° C., the mixture was stirred at 60° C. for 2 hrs. The mixture was concentrated under reduced pressure to give a residue. The residue was purified by prep-HPLC (column: Phenomenex luna C18 150*25 mm*10 μm; mobile phase: [water (FA)-ACN]; B %: 53%-83%, 11 min). Compound 1048 (30.11 mg, 44.7 μmol, 47.8% yield, 97.9% purity) was obtained as white solid. LC-MS: (M+H)⁺: 660.3. ¹H NMR: (400 MHz, DMSO-d₆) δ 12.91-12.48 (m, 1H), 8.22 (d, J=8.8 Hz, 1H), 7.97-7.89 (m, 1H), 7.86-7.73 (m, 2H), 7.67-7.60 (m, 2H), 7.51-7.42 (m, 2H), 7.41-7.28 (m, 3H), 4.73-4.59 (m, 1H), 3.71 (s, 4H), 3.27-2.88 (m, 5H), 2.80-2.64 (m, 1H), 2.49-2.34 (m, 1H), 2.28-2.10 (m, 3H), 1.22 (d, J=19.6 Hz, 3H), 0.99-0.87 (m, 1H).

### Example 67: Synthesis of Compound 1049

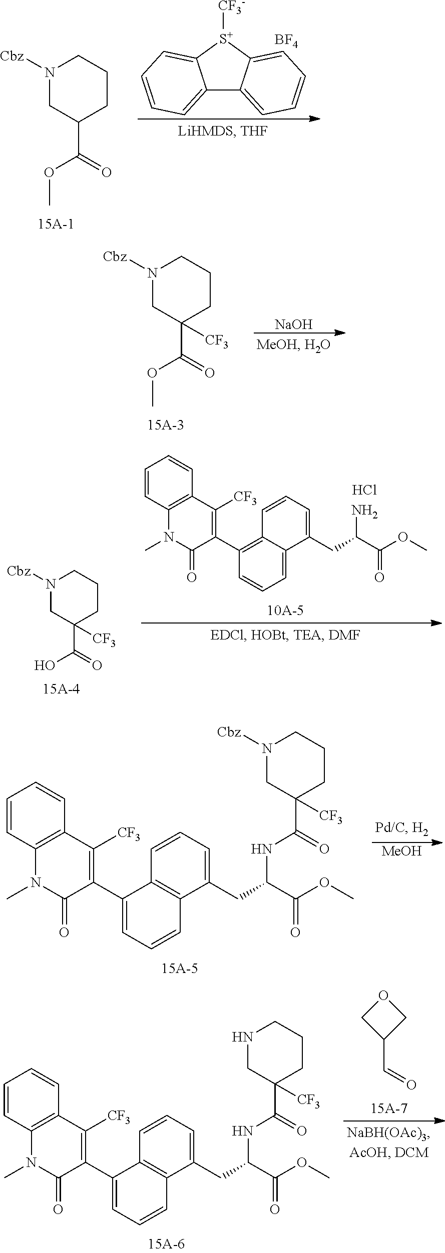

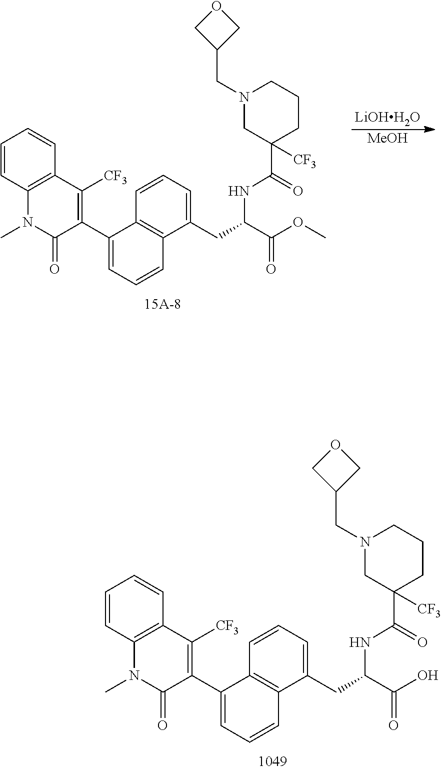 [SMILES: "MeOH" ; COC(=O)[C@H](Cc1cccc2c(-c3c(C(F)(F)F)c4ccccc4n(C)c3=O)cccc12)NC(=O)C1(C(F)(F)F)CCCN(CC2COC2)C1 ; "LiOH·H2O" ; Cn1c(=O)c(-c2cccc3c(C[C@H](NC(=O)C4(C(F)(F)F)CCCN(CC5COC5)C4)C(=O)O)cccc23)c(C(F)(F)F)c2ccccc21]

Step 1: To a solution of compound 15A-1 (500 mg, 1.80 mmol, 1.00 eq) in THF (15.0 mL) was added LiHMDS (1.00 M, 3.61 mL, 2.00 eq) at −78° C. The mixture was stirred at −78° C. for 0.5 hr, followed by adding compound 15A-2 (920 mg, 2.70 mmol, 1.50 eq) The reaction mixture was warmed to 25° C. for 12 hrs. The reaction mixture was added to 20.0 mL sat. aq. NH₄Cl, then extracted with ethyl acetate (10.0 mL*3). The combined organic layer was washed with water (10.0 mL) and brine (10.0 mL), dried over Na₂SO₄ and concentrated under reduced pressure to give a residue. The residue was purified by column (SiO₂, Petroleum ether:Ethyl acetate=1:0 to 6:1). To get a crude product compound 15A-3 (170 mg, 492 μmol, 27.3% yield) as yellow oil. ¹H NMR: (400 MHz, CDCl₃) δ 7.40-7.36 (m, 5H), 5.25 (s, 2H), 3.74 (s, 3H), 2.83 (t, J=12.0 Hz, 1H), 2.43 (d, J=13.6 Hz, 1H), 2.32 (t, J=6.0 Hz, 2H), 1.88-1.72 (m, 4H).

Step 2: To a solution of compound 15A-3 (170 mg, 492 μmol, 1.00 eq) in MeOH (5.00 mL) was added a solution of NaOH (39.3 mg, 984 μmol, 2.00 eq) in H₂O (1.00 mL) at 0° C., then the mixture was warmed to 25° C. stirred for 2 hrs. The reaction mixture was concentrated under reduced pressure to remove MeOH. The residue was diluted with H₂O 20.0 mL and extracted with DCM 20.0 mL (10.0 mL*2). The aqueous phase was adjusted pH to 6 by 1.00 M HCl under ice bath, then extracted with DCM 20.0 mL (10.0 mL*2). The combined organic layers were washed with brine 20.0 mL (10.0 mL*2), dried over Na₂SO₄, filtered, and concentrated under reduced pressure to give a residue. Compound 15A-4 (50.0 mg, 150 μmol, 30.6% yield) was obtained as white solid. LC-MS: (M−H)⁺: 329.9.

Step 3: To a solution of compound 15A-4 (50.0 mg, 150 μmol, 1.00 eq) and compound 10A-5 (74.0 mg, 150 μmol, 1.00 eq, HCl) in DMF (2.00 mL) was added EDCI (57.8 mg, 301 μmol, 2.00 eq), HOBt (40.7 mg, 301 μmol, 2.00 eq) and TEA (30.5 mg, 301 μmol, 42.0 μL, 2.00 eq). The mixture was stirred at 25° C. for 2 hrs. The reaction mixture was diluted with H₂O 15.0 mL and extracted with dichloromethane 45.0 mL (15.0 mL*3). The combined organic layers were washed with brine 45.0 mL (45.0 mL*1), dried over Na₂SO₄, filtered, and concentrated under reduced pressure to give a residue. The residue was purified by Prep-TLC (SiO₂, Dichloromethane:Methanol=5:1). Compound 15A-5 (76.0 mg, 99.0 μmol, 65.5% yield, N/A purity) was obtained as light-yellow solid. LC-MS: (M+H)⁺: 768.2. ¹H NMR: (400 MHz, CDCl₃) δ 8.21-8.10 (m, 1H), 8.05 (d, J=8.0 Hz, 1H), 7.72 (t, J=8.0 Hz, 1H), 7.64-7.58 (m, 1H), 7.54 (d, J=8.8 Hz, 1H), 7.47-7.28 (m, 10H), 5.32-5.16 (m, 1H), 5.05-4.79 (m, 2H), 4.67 (br s, 1H), 3.81 (s, 4H), 3.65-3.46 (m, 5H), 3.22-3.04 (m, 1H), 2.91-2.80 (m, 1H), 2.62 (br s, 1H), 1.70-1.59 (m, 4H)

Step 4: To a solution of compound 15A-5 (76.0 mg, 99.0 μmol, 1.00 eq) in MeOH (2.00 mL) was added Pd/C (20.0 mg, 13.0 μmol, 10.0% purity) under N₂ atmosphere. The suspension was degassed and purged with H₂ for 3 times. The mixture was stirred under H₂ (15 Psi) at 25° C. for 2 hrs. The reaction mixture was filtered and concentrated under reduced pressure to give a residue. Compound 15A-6 (60.0 mg, crude) was obtained as yellow solid. LC-MS: (M+H)⁺: 634.3.

Step 5: To a solution of compound 15A-6 (50.0 mg, 78.9 μmol, 1.00 eq) and compound 15A-7 (13.5 mg, 157 μmol, 2.00 eq) in DCM (3.00 mL) was added AcOH (23.7 mg, 394 μmol, 22.5 μL, 5.00 eq), the mixture was stirred at 25° C. for 1 hr. NaBH(OAc)₃ (33.4 mg, 157 μmol, 2.00 eq) was added. The mixture was stirred at 25° C. for 2 hrs. The reaction mixture was diluted with water 30.0 mL and extracted with DCM 90.0 mL (30.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Petroleum ether:Ethyl acetate=0:1). Compound 15A-8 (52.0 mg, 68.2 μmol, 86.4% yield, 92.3% purity) was obtained as colorless oil. LC-MS: (M+H)⁺: 704.4. ¹H NMR: (400 MHz, CDCl₃) δ 8.24-8.09 (m, 1H), 8.04 (d, J=7.2 Hz, 1H), 7.73 (t, J=7.6 Hz, 1H), 7.65-7.51 (m, 2H), 7.49-7.38 (m, 2H), 7.36-7.21 (m, 3H), 5.42-4.64 (m, 2H), 4.52-4.26 (m, 2H), 3.95-3.73 (m, 6H), 3.71-3.64 (m, 2H), 3.63-3.52 (m, 1H), 3.23-2.89 (m, 1H), 2.86-2.45 (m, 3H), 2.43-2.27 (m, 1H), 2.23-2.07 (m, 2H), 1.87-1.66 (m, 2H), 1.52-1.37 (m, 2H)

Step 6: To a solution of compound 15A-8 (45.0 mg, 59.0 μmol, 1.00 eq) in ACN (2.00 mL) was added a solution of LiOH·H₂O (2.97 mg, 70.8 μmol, 1.20 eq) in H₂O (0.500 mL), the mixture was stirred at 25° C. for 1 hr. The reaction mixture was concentrated under reduced pressure to give a residue. The residue was purified by Prep-HPLC (column: Phenomenex C18 150*25 mm*10 μm; mobile phase: [water (NH₄HCO₃)-ACN]; B %: 31%-61%, 14 min). Compound 1049 (22.97 mg, 32.7 μmol, 55.4% yield, 98.3% purity) was obtained as white solid. LC-MS: (M+H)⁺: 690.2. ¹H NMR: (400 MHz, DMSO-d₆) δ 9.32-8.78 (m, 1H), 8.30-8.11 (m, 1H), 7.93 (d, J=6.8 Hz, 1H), 7.86-7.73 (m, 2H), 7.64-7.51 (m, 1H), 7.50-7.44 (m, 1H), 7.41-7.28 (m, 4H), 4.76-4.53 (m, 2H), 4.48-4.19 (m, 2H), 4.16-3.92 (m, 1H), 3.75-3.65 (m, 4H), 3.47-3.43 (m, 1H), 3.10-2.99 (m, 1H), 2.95-2.75 (m, 1H), 2.67-2.55 (m, 2H), 2.43-2.29 (m, 1H), 2.15-2.03 (m, 1H), 2.02-1.75 (m, 2H), 1.57-1.19 (m, 3H)

### Example 68: Synthesis of Compound 1050

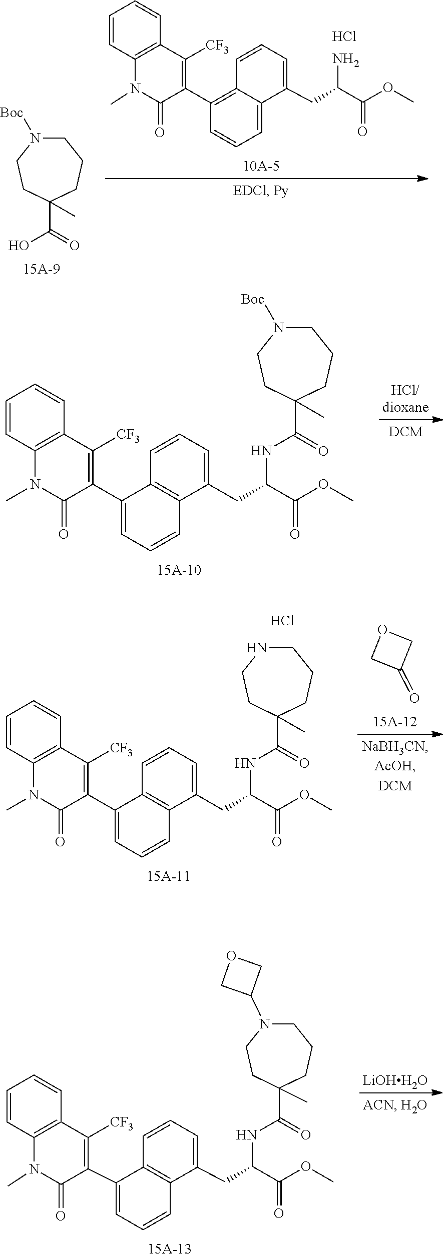

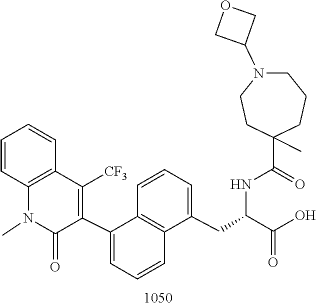 [SMILES: Cn1c(=O)c(-c2cccc3c(C[C@H](NC(=O)C4(C)CCCN(C5COC5)CC4)C(=O)O)cccc23)c(C(F)(F)F)c2ccccc21]

Step 1: To a solution of compound 15A-9 (130 mg, 505 μmol, 1.00 eq) and compound 10A-5 (248 mg, 505 μmol, 1.00 eq, HCl) in Py (3.00 mL) was added EDCI (194 mg, 1.01 mmol, 2.00 eq), the mixture was stirred at 25° C. for 2 hrs. The reaction mixture was concentrated under reduced pressure to remove Py, then diluted with saturated NH₄Cl solution 50.0 mL and extracted with dichloromethane 120 mL (40.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Dichloromethane:Methanol=10:1). Compound 15A-10 (330 mg, 474 μmol, 93.9% yield, 99.7% purity) was obtained as light-yellow solid. LC-MS: (M+H)⁺: 694.5. ¹H NMR: (400 MHz, CDCl₃) δ 8.21-8.14 (m, 1H), 8.05 (d, J=8.0 Hz, 1H), 7.73 (t, J=8.0 Hz, 1H), 7.65-7.58 (m, 1H), 7.54 (d, J=8.4 Hz, 1H), 7.48-7.38 (m, 2H), 7.36-7.28 (m, 3H), 6.02 (dd, J₁=28.4 Hz, J₂=7.6 Hz, 1H), 5.07-4.86 (m, 1H), 3.81 (s, 3H), 3.76-3.50 (m, 7H), 3.21-2.95 (m, 2H), 2.14-2.03 (m, 1H), 1.82-1.62 (m, 4H), 1.46-1.44 (m, 9H), 1.31-1.23 (m, 1H), 1.10-0.96 (m, 3H)

Step 2: To a solution of compound 15A-10 (330 mg, 474 μmol, 1.00 eq) in DCM (5.00 mL) was added HCl/dioxane (4.00 M, 2.00 mL, 16.9 eq), the mixture was stirred at 25° C. for 2 hrs. The reaction mixture was concentrated under reduced pressure to give a residue. Compound 15A-11 (280 mg, crude, HCl) was obtained as yellow solid. LC-MS: (M+H)⁺: 594.2

Step 3: To a solution of compound 15A-11 (80.0 mg, 127 μmol, 1.00 eq, HCl) and compound 15A-12 (27.5 mg, 381 μmol, 3.00 eq) in DCM (3.00 mL) was added AcOH (105 mg, 1.75 mmol, 0.100 mL, 13.8 eq), the mixture was stirred at 20° C. for 1 hr. NaBH₃CN (23.9 mg, 381 μmol, 3.00 eq) was added, the mixture was stirred at 20° C. for 2 hrs. The reaction mixture was diluted with water 30.0 mL and extracted with dichloromethane 120 mL (40.0 mL*3). The combined organic layers were washed with brine 30.0 mL (30.0 mL*1), dried over Na₂SO₄, filtered, and concentrated under reduced pressure to give a residue. The residue was purified by Prep-TLC (SiO₂, Dichloromethane:Methanol=10:1). Compound 15A-13 (74.0 mg, 114 μmol, 89.7% yield, 100% purity) was obtained as yellow oil. LC-MS: (M+H)⁺: 650.2

Step 4: To a solution of compound 15A-13 (60.0 mg, 92.4 μmol, 1.00 eq) in ACN (2.00 mL) was added a solution of LiOH·H₂O (4.65 mg, 111 μmol, 1.20 eq) in H₂O (0.500 mL), the mixture was stirred at 20° C. for 2 hrs. The residue was purified by Prep-HPLC (column: Waters xbridge 150*25 mm 10 μm; mobile phase: [water (NH₄HCO₃)-ACN]; B %: 16%-46%, 14 min). Compound 1050 (32.47 mg, 50.0 μmol, 54.2% yield, 97.9% purity) was obtained as white solid. LC-MS: (M+H)⁺: 636.3. ¹H NMR: (400 MHz, DMSO-d₆) δ 8.20 (t, J=6.8 Hz, 1H), 7.93 (d, J=7.6 Hz, 1H), 7.85-7.66 (m, 3H), 7.61 (t, J=7.2 Hz, 1H), 7.51-7.27 (m, 5H), 4.71-4.51 (m, 1H), 4.48-4.38 (m, 2H), 4.34-4.24 (m, 2H), 3.77-3.65 (m, 4H), 3.47-3.43 (m, 2H), 2.32-1.82 (m, 6H), 1.54-1.28 (m, 4H), 0.98 (d, J=5.2 Hz, 3H)

### Example 69: Synthesis of Compound 1051

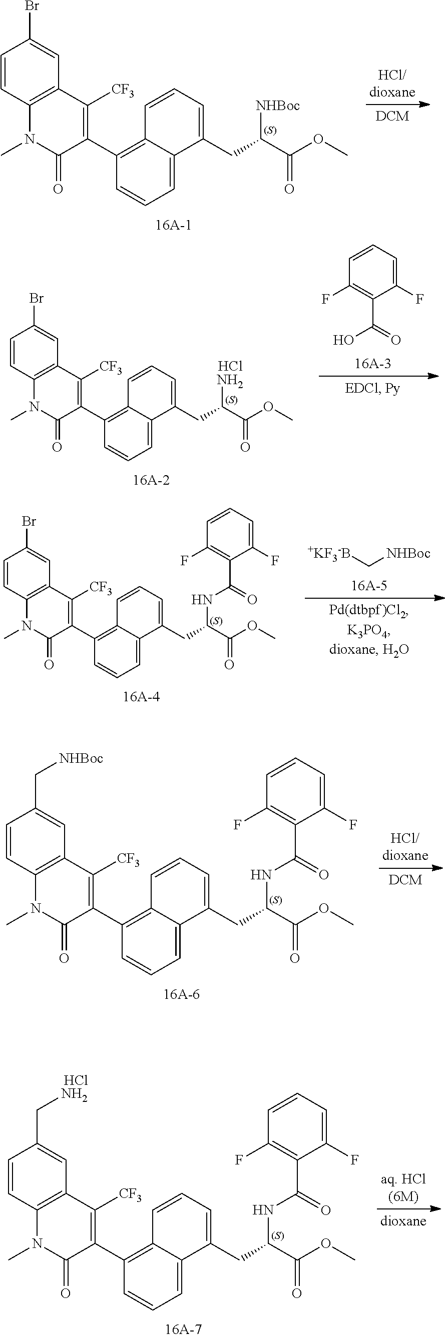

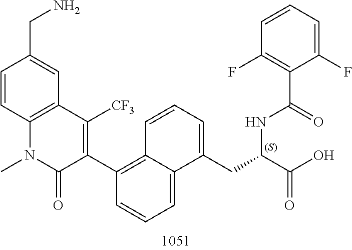

Step 1: To a solution of compound 16A-1 (600 mg, 947 μmol, 1.00 eq) in DCM (6.00 mL) was added HCl/dioxane (4.00 M, 3.60 mL, 15.2 eq). The mixture was stirred at 25° C. for 2 hrs. The reaction mixture was concentrated under reduced pressure to give a residue. Compound 16A-2 (500 mg, 877 μmol, 92.6% yield, HCl) was obtained as a white solid. LC-MS: (M-99)⁺: 534.8

Step 2: To a solution of compound 16A-2 (400 mg, 702 μmol, 1.00 eq, HCl) in Py (5.00 mL) was added EDCI (404 mg, 2.11 mmol, 3.00 eq) and compound 16A-3 (111 mg, 702 μmol, 1.00 eq). The mixture was stirred at 25° C. for 2 hrs. The reaction mixture was concentrated under reduced pressure. 50.0 mL of water was added. The aqueous layer was diluted with ethyl acetate 20.0 mL and extracted with ethyl acetate (20.0 mL*3). The combined organic layers were washed with brine (20.0 mL*4), dried over Na₂SO₄ filtered and concentrated under reduced pressure to give a residue. The residue was purified by Prep-TLC (SiO₂, Petroleum ether:Ethyl acetate=1:1). Compound 16A-4 (400 mg, 594 μmol, 84.6% yield) was obtained as a yellow solid. LC-MS: (M+H)⁺: 673.3. ¹H NMR: (400 MHz, DMSO_d₆) δ 9.41 (dd, J=7.6, 10.0 Hz, 1H), 8.17 (dd, J₁=8.2 Hz, J₂=4.0 Hz, 1H), 7.99 (dd, J₁=9.6 Hz, J₂=1.6 Hz, 1H), 7.94 (brd, J=1.2 Hz, 1H), 7.75 (d, J=8.8 Hz, 1H), 7.67-7.63 (m, 1H), 7.53-7.50 (m, 1H), 7.48-7.40 (m, 2H), 7.37-7.33 (m, 2H), 7.17-7.12 (m, 2H), 4.85-4.78 (m, 1H), 3.70-3.63 (m, 7H), 3.49-3.42 (m, 1H)

Step 3: To a mixture of compound 16A-4 (120 mg, 178 μmol, 1.00 eq) and compound 16A-5 (46.5 mg, 196 μmol, 1.10 eq) in dioxane (3.00 mL) and H₂O (0.500 mL) was added Pd (dtbpf) C12 (11.6 mg, 17.8 μmol, 0.100 eq) and K₃PO₄ (113 mg, 535 μmol, 3.00 eq). The mixture was stirred at 25° C. for 2 hrs. The reaction mixture was quenched by addition water 20.0 mL, and then diluted with ethyl acetate 10.0 mL and extracted with ethyl acetate (10.0 mL*3). The combined organic layers were washed with brine (10.0 mL*4), dried over Na 2SO₄ filtered and concentrated under reduced pressure to give a residue. The residue was purified by Prep-TLC (SiO₂, Dichloromethane:Methanol=10:1). Compound 16A-6 (90.0 mg, 124 μmol, 69.8% yield) was obtained as a yellow solid. LC-MS: (M+H)⁺: 724.2

Step 4: To a solution of compound 16A-6 (90.0 mg, 124 μmol, 1.00 eq) in DCM (2.00 mL) was added HCl/dioxane (4.00 M, 31.1 μL, 1.00 eq). The mixture was stirred at 25° C. for 2 hrs. The reaction mixture was concentrated under reduced pressure to give a residue. Compound 16A-7 (90.0 mg, crude, HCl) was obtained as yellow solid. ¹H NMR: (400 MHz, DMSO_d₆) δ 9.43 (br dd, J₁=11.2 Hz, J₂=7.6 Hz, 1H), 8.34 (brd, J=1.6 Hz, 2H), 8.23-8.13 (m, 1H), 8.02 (br s, 1H), 7.96-7.89 (m, 1H), 7.88-7.83 (m, 1H), 7.68-7.84 (m, 1H), 7.53-7.33 (m, 4H), 7.17-7.12 (m, 2H), 4.85-4.78 (m, 1H), 4.22 (br d, J=5.6 Hz, 1H), 3.78-3.73 (m, 3H), 3.65 (d, J=12.0 Hz, 2H), 3.57 (s, 3H)

Step 5: To a solution of compound 16A-7 (70.0 mg, 106 μmol, 1.00 eq, HCl) in dioxane (1.00 mL) was added HCl (4.00 M, 2.00 mL, 75.4 eq). The mixture was stirred at 60° C. for 1 hr. The reaction mixture was concentrated under reduced pressure to give a residue. The residue was purified by Prep-HPLC (column: Phenomenex luna C18 150*25 mm*10 μm; mobile phase: [water (FA)-ACN]; B %: 13%-43%, 58 min). Compound 1051 (32.65 mg, 52.07 μmol, 49.09% yield, 97.2% purity) was obtained as a white solid. LC-MS: (M+H)⁺: 610.3. ¹H NMR: (400 MHz, DMSO_d₆) δ 8.77-8.73 (m, 1H), 8.33-8.28 (m, 1H), 7.97 (br s, 1H), 7.88-7.86 (m, 1H), 7.79-7.77 (m, 1H), 7.62-7.57 (m, 1H), 7.52-7.43 (m, 1H), 7.41-7.40 (m, 1H), 7.34-7.24 (m, 3H), 7.11 (brt, J=8.0 Hz, 2H), 4.61-4.58 (m, 1H), 4.08 (br s, 2H), 3.71 (br d, J=4.0 Hz, 4H)

Compound 1052 was synthesized analogous to 1051. A general scheme for the synthesis of 1052 is provided below. Spectroscopic data is provided in Table 28.

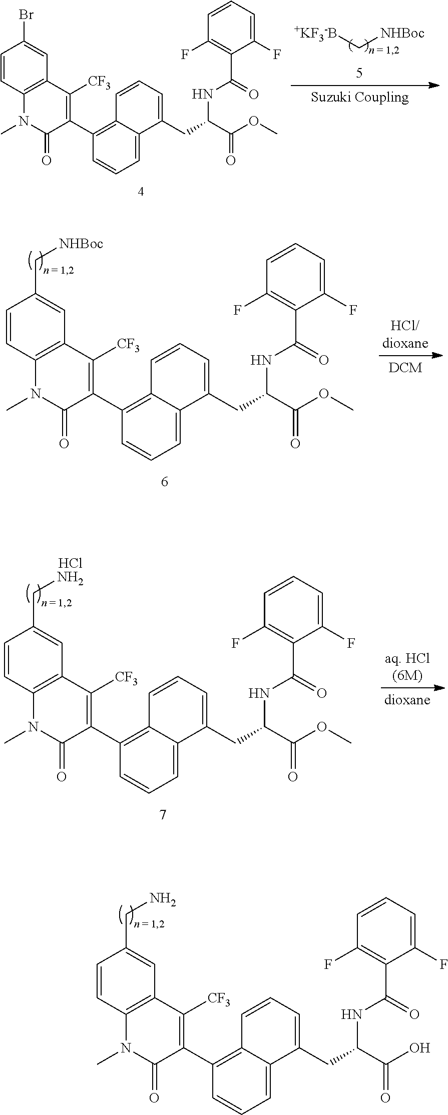

TABLE 28
Structure and Spectroscopic data for Compound 1052

| No. | Structure |
|---|---|
| 1052 | 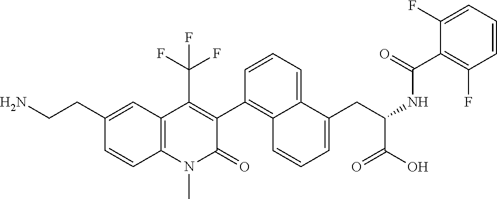 [SMILES: Cn1c(=O)c(-c2cccc3c(C[C@H](NC(=O)c4c(F)cccc4F)C(=O)O)cccc23)c(C(F)(F)F)c2cc(CCN)ccc21] |
| | m/z = 624.6 |

### Example 70: Synthesis of Compound 1053

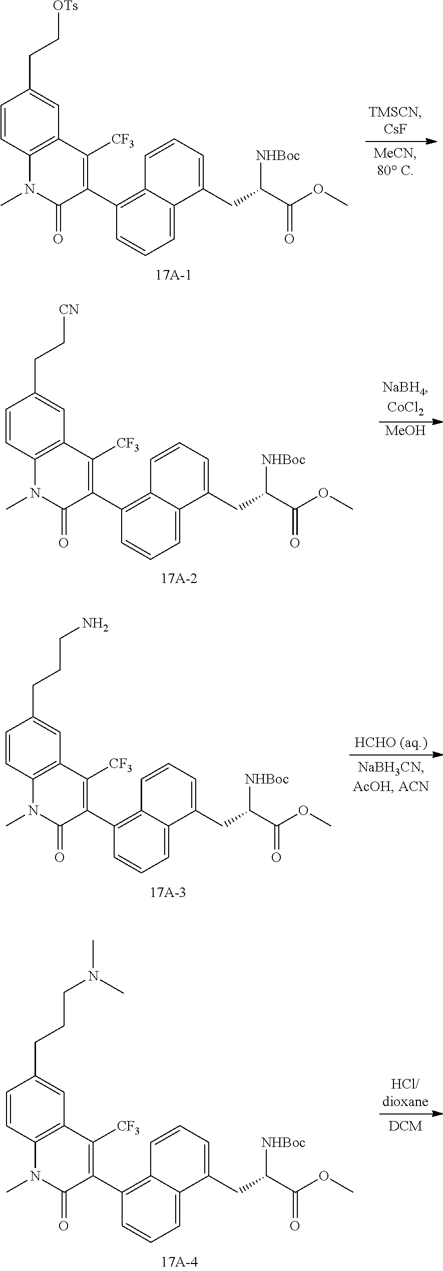 [SMILES: COC(=O)[C@H](Cc1cccc2c(-c3c(C(F)(F)F)c4cc(CCOS(=O)(=O)c5ccc(C)cc5)ccc4n(C)c3=O)cccc12)NC(=O)OC(C)(C)C ; "MeOH" ; COC(=O)[C@H](Cc1cccc2c(-c3c(C(F)(F)F)c4cc(CCC#N)ccc4n(C)c3=O)cccc12)NC(=O)OC(C)(C)C ; COC(=O)[C@H](Cc1cccc2c(-c3c(C(F)(F)F)c4cc(CCCN)ccc4n(C)c3=O)cccc12)NC(=O)OC(C)(C)C ; COC(=O)[C@H](Cc1cccc2c(-c3c(C(F)(F)F)c4cc(CCCN(C)C)ccc4n(C)c3=O)cccc12)NC(=O)OC(C)(C)C]

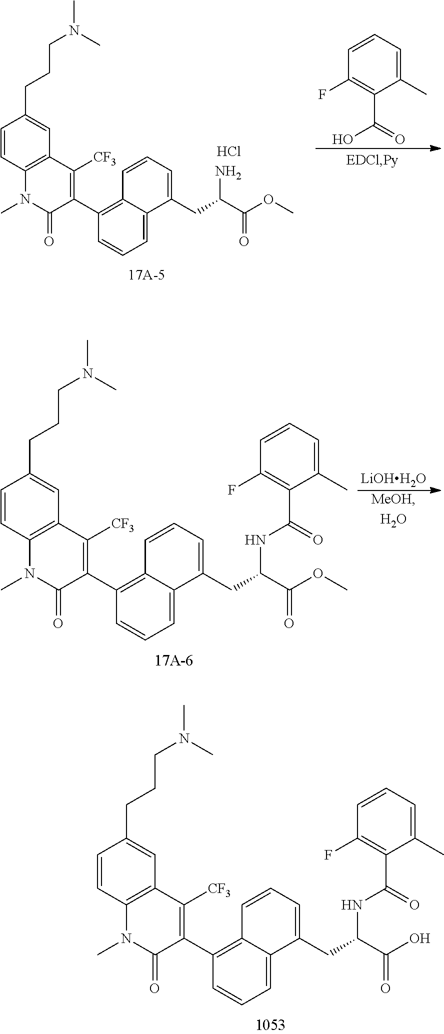

Step 1: To a solution of compound 17A-1 (110 mg, 143 μmol, 1.00 eq) in ACN (2.00 mL) was added CsF (65.3 mg, 430 μmol, 15.9 μL, 3.00 eq) and TMSCN (28.4 mg, 287 μmol, 35.9 μL, 2.00 eq). The mixture was stirred at 80° C. for 3 hrs. The reaction mixture was quenched by addition sat. NaHCO₃20.0 mL at 0° C., and then diluted with H₂O 30.0 mL and extracted with ethyl acetate 45.0 mL (15.0 mL*3). The combined organic layers were dried over Na₂SO₄, filtered, and concentrated under reduced pressure to give a residue. The residue was purified by prep-TLC (SiO₂, DCM/MeOH=10/1). Compound 17A-2 (85.0 mg, 140 μmol, 97.6% yield) was obtained as a white solid. LC-MS: (M+Na)⁻: 630.3. ¹H NMR: (400 MHz, DMSO-d₆) δ 8.16-8.07 (m, 1H), 7.86-7.73 (m, 3H), 7.68-7.58 (m, 1H), 7.51-7.30 (m, 5H), 4.43-4.28 (m, 1H), 3.71 (s, 3H), 3.65-3.56 (m, 4H), 3.38-3.37 (m, 1H), 3.06 (t, J=6.8 Hz, 2H), 2.89 (t, J=6.4 Hz, 2H), 1.40-1.20 (m, 9H).

Step 2: To a solution of compound 17A-2 (85.0 mg, 140 μmol, 1.00 eq) in MeOH (1.00 mL) was added CoCl₂ (1.82 mg, 14.0 μmol, 0.100 eq) and NaBH₄ (7.49 mg, 198 μmol, 1.42 eq) at 0° C. The mixture was stirred at 25° C. for 2 hrs. The reaction mixture was quenched by addition sat. NH₄Cl 20.0 mL at 0° C., and then diluted with H₂O 30.0 mL and extracted with ethyl acetate 45.0 mL (15.0 mL*3). The combined organic layers were concentrated under reduced pressure to give a residue. The residue was purified by prep-TLC (SiO₂, DCM/MeOH=10/1). Compound 17A-3 (50.0 mg, 81.8 μmol, 58.4% yield) was obtained as a white solid. LC-MS: (M+H)⁺: 612.3. ¹H NMR: (400 MHz, DMSO-d₆) δ 8.20-8.01 (m, 1H), 7.76-7.58 (m, 4H), 7.51-7.29 (m, 5H), 4.42-4.28 (m, 1H), 3.71 (s, 3H), 3.67-3.56 (m, 4H), 3.19-3.16 (m, 1H), 2.80 (q, J=8.0 Hz, 4H), 1.92-1.80 (m, 2H), 1.38-1.20 (m, 9H).

Step 3: To a solution of compound 17A-3 (45.0 mg, 73.6 μmol, 1.00 eq) in CH₃COOH (0.500 mL) and ACN (0.500 mL) was added HCHO (59.7 mg, 736 μmol, 54.8 μL, 37.0% purity, 10.0 eq) and NaBH₃CN (6.94 mg, 110 μmol, 1.50 eq). The mixture was stirred at 25° C. for 1 hr. The reaction mixture was diluted with H₂O 50.0 mL and extracted with ethyl acetate 45.0 mL (15.0 mL*3). The combined organic layers were concentrated under reduced pressure to give a residue. Compound 17A-4 (48.0 mg, crude) was obtained as a yellow oil.

Step 4: To a solution of compound 17A-4 (48.0 mg, 75.0 μmol, 1.00 eq) in DCM (0.500 mL) was added HCl/dioxane (4.00 M, 0.500 mL, 26.7 eq). The mixture was stirred at 25° C. for 0.5 hr. The reaction mixture was concentrated under reduced pressure to give a residue. Compound 17A-5 (45.0 mg, crude, HCl) was obtained as a yellow oil. LC-MS: (M+H)⁺: 540.2.

Step 5: To a solution of compound 17A-5 (40.0 mg, 40.1 μmol, 1.00 eq, HCl), 2-fluoro-6-methyl-benzoic acid (6.81 mg, 44.15 μmol, 1.1 eq) in Py (0.500 mL) was added EDCI (15.4 mg, 80.3 μmol, 2.00 eq). The mixture was stirred at 25° C. for 0.5 hr. The reaction mixture was quenched by addition sat. NH₄Cl 30.0 mL at 25° C., and then diluted with H₂O 20.0 mL and extracted with ethyl acetate 45.0 mL (15.0 mL*3). The combined organic layers were dried over Na₂SO₄, filtered, and concentrated under reduced pressure to give a residue. The residue was purified by prep-TLC (SiO₂, DCM/MeOH=10/1). Compound 17A-6 (27.0 mg, crude) was obtained as a yellow oil. LC-MS: (M+H)⁺: 676.5. ¹H NMR: (400 MHz, DMSO-d₆) δ 9.28-9.05 (m, 1H), 8.30-8.00 (m, 1H), 7.80-7.61 (m, 4H), 7.50-7.23 (m, 5H), 7.03 (t, J=8.4 Hz, 2H), 4.93-4.80 (m, 1H), 4.13-4.06 (m, 1H), 3.72-3.70 (m, 3H), 3.68 (d, J=3.6 Hz, 3H), 3.46-3.45 (m, 1H), 2.78-2.73 (m, 2H), 2.32-2.27 (m, 2H), 2.19 (br s, 6H), 2.11-1.98 (m, 3H), 1.82-1.72 (m, 2H).

Step 6: To a solution of compound 17A-6 (27.0 mg, 40.0 μmol, 1.00 eq) in MeOH (0.500 mL) was added LiOH H₂O (3.35 mg, 80.0 μmol, 2.00 eq) in H₂O (0.500 mL). The mixture was stirred at 25° C. for 0.5 hr. The reaction mixture was concentrated under reduced pressure to give a residue. The residue was purified by prep-HPLC (column: Welch Xtimate C18 150*25 mm*5 μm; mobile phase: [water (NH₃H₂O)-ACN]; B %: 18%-48% B over min). Compound 1053 (5.33 mg, 7.88 μmol, 19.7% yield, 97.8% purity) was obtained as an off-white solid. LC-MS: (M+H)⁺: 662.2. ¹H NMR: (400 MHz, DMSO-d₆) δ 8.40 (d, J=8.4 Hz, 1H), 8.12-7.97 (m, 1H), 7.69 (br s, 3H), 7.57 (t, J=7.6 Hz, 1H), 7.44 (t, J=5.6 Hz, 1H), 7.34-7.21 (m, 4H), 7.03-6.94 (m, 2H), 4.63-4.38 (m, 1H), 3.76-3.73 (m, 1H), 3.71 (s, 3H), 3.27-3.25 (m, 1H), 2.75 (t, J=6.8 Hz, 2H), 2.23 (t, J=6.8 Hz, 2H), 2.18-2.03 (m, 9H), 1.81-1.69 (m, 2H).

### Example 71: Inhibition of α4β7 and α4β1 Integrins

α4β7 and α4β1 inhibition was evaluated in vitro using at least one of the following procedures:

### Integrin Adhesion Assay: Protocol 1

MAdCAM (R&D Systems, 0.1 ug) was diluted in 50 μL PBS, added to each well of an opaque ELISA plate (Thermo), and incubated overnight at 4° C. Coated plates were washed once with PBS and then blocked with 200 μL of assay buffer (10 mM HEPES, 150 mM NaCl, 1 mM MnCl₂, 0.1 mM CaCl₂), 1% BSA) for 1 hour at 37° C. and 5% CO₂. After incubation, buffer was aspirated from the plates and 50 μL of fresh assay buffer is added. Compounds were added by a compouns dispenser (Tecan) and DMSO concentration was normalized to 1% across each plate. 50 μL of RPMI-8866 cells (2×10⁶ per mL) were added to each well and the plates were incubated for 1 hour at 37° C. and 5% CO₂. After incubation, plates were allowed to cool to RT for 10 minutes before being washed four times in assay buffer by an automated plate washer (BioTek). After washing, 100 μL of a 1:1 mixture of assay buffer and CellTiter GLO 2.0 reagent (Promega) were added to each well. Plates were mixed for 2 minutes at 1000 rpm and then allowed to incubate an additional 10 minutes before reading out luminescence on a Tecan Spark plate reader. Raw data was converted to percent inhibition based on DMSO only and control compound wells, and curves were analyzed by 4-parameter fit within Dotmatics software.

### Integrin Adhesion Assay: Protocol 2

VCAM (R&D Systems, 0.1 ug) was diluted in 50 μL PBS, added to each well of an opaque ELISA plate (Thermo), and incubated overnight at 4° C. Coated plates were washed once with PBS and then blocked with 200 μL of assay buffer (10 mM HEPES, 150 mM NaCl, 1 mM MnCl₂, 0.1 mM CaCl₂), 1% BSA) for 1 hour at 37° C. and 5% CO₂. After incubation, buffer was aspirated from the plates and 50 μL of fresh assay buffer was added. Compounds were added by compounds dispenser (Tecan) and DMSO concentration was normalized to 1% across each plate. 50 μL of Jurkat cells (2×10⁶ per mL) were added to each well and the plates were incubated for 1 hour at 37° C. and 5% CO₂. After incubation, plates were allowed to cool to RT for 10 minutes before being washed four times in assay buffer by an automated plate washer (BioTek). After washing, 100 μL of a 1:1 mixture of assay buffer and CellTiter GLO 2.0 reagent (Promega) were added to each well. Plates were mixed for 2 minutes at 1000 rpm and then allowed to incubate an additional 10 minutes before reading out luminescence on a Tecan Spark plate reader. Raw data was converted to percent inhibition based on DMSO only and control compound wells, and curves were analyzed by 4-parameter fit within Dotmatics software.

### Integrin Adhesion Assay: Protocol 3

Either MAdCAM or VCAM (R&D Systems, 0.1 ug) was diluted in 50 μL PBS, added to each well of an opaque ELISA plate (Thermo), and incubated overnight at 4° C. Coated plates were washed once with PBS and then blocked with 200 μL of assay buffer (10 mM HEPES, 150 mM NaCl, 1% BSA) for 1 hour at 37° C. and 5% CO₂. After incubation, buffer was aspirated from the plates and 50 μL of fresh assay buffer was added. Compounds were added by compounds dispenser (Tecan) and DMSO concentration is normalized to 1% across each plate. 50 μL of RPMI-8866 cells (2×10⁶ per mL) were added to each well and the plates were incubated for 1 hour at 37° C. and 5% CO₂. After incubation, plates were washed four times in assay buffer by an automated plate washer (BioTek). After washing, 100 μL of a 1:1 mixture of assay buffer and CellTiter GLO 2.0 reagent (Promega) were added to each well. Plates were mixed for 2 minutes at 1000 rpm and then allowed to incubate an additional 10 minutes before reading out luminescence on a Tecan Spark plate reader. Raw data was converted to percent inhibition based on DMSO only and control compound wells, and curves were analyzed by 4-parameter fit within Dotmatics software.

Integrin Adhesion Assay Using Genetically Modified Cells Generating Stable Cell Lines with ITGB1 or ITGB7 Knockout (Protocol 4 and Protocol 5)

To obtain subclones of the RPMI-8866 cell line (Sigma-Aldrich, St. Louis, MO) that were individually deficient in either integrin β1 or integrin β 7, CRISPR/Cas9 technology was employed (PMID: 30108345). Specifically, a Neon™ Transfection System System 10 μL Kit and the Neon™ Transfection device (both from ThermoFisher Scientific) were used.

The protocol was performed following the manufacturer instructions, described in https://www.thermofisher.com/document-connect/document-connect.html?url=https % 3A%2F%2F assets.thermofisher.com%2FTFS-Assets%2FLSG%2Fmanuals%2FMAN0017066_TrueCut Cas9_Protein_v2_UG.pdf, which is incorporated by reference herein in its entirety.

RPMI-8866 cells were transferred to a flask the day before transfection, at a confluence of 5×10⁵ cells/mL. The cells were washed with PBS and resuspended in buffer R (Neon™ Transfection System, Invitrogen) to 2×10⁵ cells/10 uL for each reaction. The TrueCut™ HiFi Cas9 Protein (1250 ng per reaction) was mixed with TrueGuide™ sgRNA (7.5 pmoles each/pool of 3 sequences, table 1) (ThermoFisher Scientific). This mix was combined with the RPMI-8866 cells and electroporated on the Neon system at 1700V/20 ms/1 pulse. Transfected cells were allowed to recover in 500 μL pre-warmed cRMPI media (RPMI-1640 media with 10% FBS, 100 U/ml Penicillin-Streptomycin) at 37° C., 5% CO₂ for 24 hours. At that time, an extra 500 μL of pre-warmed cRMPI media was added, and cells were incubated for another 24 hours.

For flow cytometry analysis, 100 μL of the transfected cells were collected and stained with anti-itga4 (Clone: 9F10, PE, BD Pharmingen™), anti-itgb 1 (Clone: (TS2/16), Super Bright 436, eBioscience), anti-itgb7 (Clone: FIB504, Brilliant Violet 650, BD Pharmingen™) and viability dye (LIVE/DEAD™ Fixable Green Dead Cell Stain, Invitrogen™). An Attune NxT Flow Cytometer was used to analyze the CRISPR-transfected cells. The flow cytometry results were analyzed using FlowJo™ v10.8.1 Software (BD Life Sciences). Fluorescence minus one (FMO) controls were used to select appropriate gates for data analysis.

To generate an individual clones of cells deficient in either ITGB7 or ITGB1 by limiting dilution assay, the transfected cells were resuspended at 5 cells/mL and seeded in 384 well plates. Outgrowth of clones was monitored using a microscope, and 24 clones were transferred for expansion. Expression of either integrin was verified by flow cytometry in each of the clones. One clone of each integrin b7 or the integrin b1 knockout was chosen, expanded and aliquots were frozen in vapor phase liquid nitrogen.

### Integrin Adhesion Assay: Protocol 4

MAdCAM (R&D Systems, 0.1 ug) was diluted in 50 μL PBS, added to each well of an opaque ELISA plate (Thermo), and incubated overnight at 4° C. Coated plates were washed once with PBS and then blocked with 200 μL of assay buffer (10 mM HEPES, 150 mM NaCl, 1 mM MnCl₂, 0.1 mM CaCl₂), 1% BSA) for 1 hour at 37° C. and 5% CO₂. After incubation, buffer was aspirated from the plates and 50 μL of fresh assay buffer was added. Compounds were added using a compounds dispenser (Tecan), and DMSO concentration was normalized to 1% across each plate. 50 μL of RPMI-8866 cells with a stable loss of function (LOF) mutation of the ITGB1 gene (2×10⁶ per mL) were added to each well and the plates were incubated for 1 hour at 37° C. at a CO₂ concentration of 5%. After incubation, plates were allowed to cool to RT for 10 minutes before being washed four times in assay buffer by an automated plate washer (BioTek). After washing, 100 μL of a 1:1 mixture of assay buffer and CellTiter GLO 2.0 reagent (Promega) were added to each well. Plates were mixed for 2 minutes at 1000 rpm and then allowed to incubate an additional 10 minutes before reading out luminescence on a Tecan Spark plate reader. Raw data was converted to percent inhibition based on DMSO only and control compound conditions, and curves were fitted by 4-parameter curves within Dotmatics software.

### Integrin Adhesion Assay: Protocol 5

VCAM (R&D Systems, 0.1 ug) was diluted in 50 μL PBS, added to each well of an opaque ELISA plate (Thermo), and incubated overnight at 4° C. Coated plates were washed once with PBS and then blocked with 200 μL of assay buffer (10 mM HEPES, 150 mM NaCl, 1 mM MnCl₂, 0.1 mM CaCl₂), 1% BSA) for 1 hour at 37° C. and 5% CO₂. After incubation, buffer was aspirated from the plates and 50 μL of fresh assay buffer was added. Compounds were added by compounds dispenser (Tecan) and DMSO concentration was normalized to 1% across each plate. 50 μL of RPMI-8866 cells with a stable LOF mutation of the ITGB7 gene (2×10⁶ per mL) were added to each well and the plates were incubated for 1 hour at 37° C. and 5% CO₂. After incubation, plates were allowed to cool to RT for 10 minutes before being washed four times in assay buffer by an automated plate washer (BioTek). After washing, 100 μL of a 1:1 mixture of assay buffer and CellTiter GLO 2.0 reagent (Promega) were added to each well. Plates were mixed for 2 minutes at 1000 rpm and then allowed to incubate an additional 10 minutes before reading out luminescence on a Tecan Spark plate reader. Raw data was converted to percent inhibition based on DMSO only and control compound conditions, and curves were fitted by 4-parameter curves within Dotmatics software.

TABLE 29
Single guided RNAs (ThermoFisher Scientific) pools

| | |
|---|---|
| itgb7 pool | TrueGuide ™ Synthetic sgRNA CRISPR646260_SGMITGB7 |
| | Human |
| | TrueGuide ™ Synthetic sgRNA CRISPR646267_SGMITGB7 |
| | Human |
| | TrueGuide ™ Synthetic sgRNA CRISPR646307_SGMITGB7 |
| | Human |
| itgb1 pool | TrueGuide ™ Synthetic sgRNA CRISPR626332_SGMITGB1 |
| | Human |
| | TrueGuide ™ Synthetic sgRNA CRISPR626355_SGMITGB1 |
| | Human |
| | TrueGuide ™ Synthetic sgRNA CRISPR469451_SGMITGB1 |
| | Human |

Table 30 and Table 31 below, provides IC₅₀ values for the inhibition data for compounds of the present disclosure as determined by at least one of the assays described above.

TABLE 30

| Compound No. | Mean IC₅₀ α4β7 | Mean IC₅₀ α4β1 |
|---|---|---|
| 1 | B | C |
| 2 | A | D |
| 3 | B | D |
| 4 | B | D |
| 5 | B | — |
| 6 | A | D |
| 7 | B | D |
| 8 | A | C |
| 9 | A | D |
| 10 | B | C |
| 11 | C | — |
| 12 | A | C |
| 13 | B | — |
| 14 | A | D |
| 15 | A | C |
| 16 | A | C |
| 17 | B | D |
| 18 | B | D |
| 19 | A | C |
| 20 | A | C |
| 21 | B | D |
| 22 | A | D |
| 23 | A | D |
| 24 | D | D |
| 25 | D | D |
| 26 | B | D |
| 27 | B | D |
| 28 | B | D |
| 29 | B | D |
| 30 | B | D |
| 31 | B | C |
| 32 | B | — |
| 33 | D | D |
| 34 | C | D |
| 35 | D | D |
| 36 | D | D |
| 37 | D | D |
| 38 | C | D |
| 39 | B | D |
| 43 | A | C |
| 44 | D | D |
| 45 | D | D |
| 46 | A | D |
| 47 | B | D |
| 48 | A | C |
| 49 | D | D |
| 50 | C | D |
| 51 | C | D |
| 52 | C | D |
| 53 | C | D |
| 54 | D | D |
| 55 | C | D |
| 56 | C | D |
| 57 | B | D |
| 58 | B | D |
| 59 | B | D |
| 60 | B | D |
| 62 | D | D |
| 63 | B | D |
| 64 | D | D |
| 65 | C | C |
| 66 | C | D |
| 67 | A | D |
| 68 | D | D |
| 69 | D | D |
| 70 | D | D |
| 71 | D | D |
| 72 | C | D |
| 76 | D | D |
| 79 | B | D |
| 80 | D | D |
| 108 | A | C |
| 109 | C | — |
| 110 | B | C |
| 111 | A | C |
| 112 | A | D |
| 113 | A | C |
| 114 | A | D |
| 115 | B | C |
| 116 | B | D |
| 117 | B | D |
| 118 | B | D |
| 119 | B | D |
| 120 | B | D |
| 121 | D | — |
| 122 | B | — |
| 123 | B | — |
| 124 | B | D |
| 125 | D | — |
| 126 | A | C |
| 127 | D | D |
| 128 | C | D |
| 129 | A | C |
| 130 | A | D |
| 131 | B | C |
| 132 | A | C |
| 133 | A | D |
| 134 | A | — |
| 136 | B | — |
| 137 | B | D |
| 138 | B | D |
| 139 | B | C |
| 140 | C | D |
| 141 | B | D |
| 142 | B | D |
| 143 | A | C |
| 144 | B | C |
| 146 | A | D |
| 148 | A | D |
| 149 | B | D |
| 150 | A | D |
| 151 | A | D |
| 152 | B | C |
| 153 | B | C |
| 154 | D | D |
| 155 | D | D |
| 156 | C | D |
| 157 | B | D |
| 158 | C | D |
| 159 | C | D |
| 160 | B | D |
| 161 | C | D |
| 162 | C | D |
| 163 | B | D |
| 164 | D | D |
| 165 | C | D |
| 166 | D | D |
| 167 | A | C |
| 168 | C | D |
| 169 | A | D |
| 170 | B | D |
| 171 | A | C |
| 172 | B | C |
| 176 | C | D |
| 177 | D | D |
| 178 | D | D |
| 179 | D | D |
| 210 | A | D |
| 211 | B | — |
| 212 | C | — |
| 213 | B | D |
| 214 | A | D |
| 215 | A | D |
| 216 | B | D |
| 217 | A | D |
| 218 | B | D |
| 220 | B | D |
| 222 | A | D |
| 223 | D | D |
| 224 | B | D |
| 225 | A | D |
| 226 | A | D |
| 227 | D | D |
| 229 | D | D |
| 237 | A | D |
| 245 | A | D |
| 246 | C | D |
| 248 | A | D |
| 249 | B | D |
| 251 | A | D |
| 252 | A | D |
| 253 | A | D |
| 254 | A | D |
| 255 | A | C |
| 256 | B | D |
| 257 | B | D |
| 258 | C | D |
| 259 | A | C |
| 260 | B | C |
| 281 | B | D |
| 282 | C | D |

A = X < 10 nM;
B = 10 nM < X < 100 nM;
C = 100 nM < X < 1000 nM;
D = 1000 nM < X

TABLE 31

| Comp. | Mean | Mean | Mean |
|---|---|---|---|
| No. | IC₅₀ α4β7 | IC₅₀ α4β7 KO^{a} | IC₅₀ α4β1 KO^{b} |
| 291 | C | C | — |
| 292 | C | C | — |
| 293 | — | — | — |
| 294 | A | — | D |
| 295 | — | D | — |
| 296 | — | — | — |
| 297 | — | — | — |
| 298 | A | — | D |
| 299 | A | — | D |
| 300 | A | — | D |
| 301 | B | — | D |
| 302 | B | — | D |
| 303 | D | — | — |
| 304 | D | — | — |
| 305 | C | D | — |
| 306 | D | — | — |
| 307 | C | C | D |
| 308 | B | — | D |
| 309 | B | — | D |
| 310 | C | C | — |
| 311 | C | C | — |
| 312 | B | — | D |
| 313 | B | — | D |
| 314 | B | — | D |
| 315 | B | — | D |
| 316 | C | B | D |
| 317 | B | — | D |
| 318 | C | — | D |
| 319 | C | — | — |
| 320 | C | — | — |
| 321 | B | — | D |
| 322 | B | — | C |
| 323 | B | — | D |
| 324 | B | — | D |
| 325 | D | — | D |
| 326 | D | — | — |
| 327 | C | — | D |
| 328 | C | C | — |
| 329 | D | — | — |
| 330 | B | — | D |
| 331 | B | — | D |
| 332 | C | C | — |
| 333 | D | — | — |
| 334 | D | — | — |
| 335 | C | C | — |
| 336 | C | C | — |
| 337 | C | — | — |
| 338 | D | — | — |
| 339 | C | C | — |
| 340 | C | — | — |
| 341 | C | C | — |
| 342 | D | — | — |
| 343 | D | — | — |
| 344 | D | — | — |
| 345 | D | — | — |
| 346 | B | — | D |
| 347 | B | — | D |
| 348 | C | — | — |
| 349 | D | — | — |
| 350 | B | — | D |
| 351 | A | — | D |
| 352 | B | B | C |
| 353 | B | — | D |
| 354 | C | C | D |
| 355 | B | B | D |
| 356 | B | — | D |
| 357 | B | — | D |
| 358 | B | — | D |
| 359 | A | — | D |
| 360 | A | — | D |
| 361 | B | — | D |
| 362 | B | — | D |
| 363 | B | — | D |
| 364 | C | — | D |
| 365 | C | C | — |
| 366 | C | C | — |
| 367 | C | C | — |
| 368 | C | C | — |
| 369 | D | — | — |
| 370 | D | — | — |
| 371 | C | C | — |
| 372 | C | C | — |
| 373 | C | C | D |
| 374 | C | C | — |
| 375 | C | — | D |
| 376 | D | — | — |
| 377 | D | — | — |
| 378 | B | — | D |
| 379 | C | — | — |
| 380 | B | — | D |
| 381 | C | — | D |
| 382 | C | C | — |
| 383 | C | — | — |
| 384 | C | — | D |
| 385 | B | — | D |
| 386 | C | C | — |
| 387 | C | C | — |
| 388 | C | C | — |
| 389 | C | C | — |
| 390 | C | C | — |
| 391 | D | — | — |
| 392 | B | — | D |
| 393 | B | — | D |
| 394 | B | — | — |
| 395 | C | — | — |
| 396 | C | C | — |
| 397 | C | — | D |
| 398 | B | — | — |
| 399 | C | B | — |
| 400 | C | C | — |
| 401 | B | — | D |
| 402 | D | — | D |
| 403 | B | B | D |
| 404 | C | C | — |
| 405 | B | — | D |
| 406 | D | — | — |
| 407 | B | — | D |
| 408 | B | — | D |
| 409 | C | C | — |
| 410 | C | C | |
| 411 | C | C | D |
| 412 | D | — | D |
| 413 | B | — | D |
| 414 | A | — | D |
| 415 | C | — | D |
| 416 | C | C | — |
| 417 | D | — | — |
| 418 | A | — | D |
| 419 | B | B | D |
| 420 | B | — | D |
| 421 | — | A | D |
| 422 | B | C | D |
| 423 | C | C | — |
| 424 | A | — | D |
| 425 | C | C | — |
| 426 | C | C | — |
| 427 | D | — | — |
| 428 | D | — | — |
| 429 | D | — | — |
| 430 | — | A | C |
| 431 | A | — | C |
| 432 | — | A | B |
| 433 | A | A | D |
| 434 | — | — | D |
| 435 | — | — | — |
| 436 | — | — | — |
| 437 | D | — | — |
| 438 | C | B | — |
| 439 | B | — | D |
| 440 | B | — | D |
| 441 | B | — | — |
| 442 | C | C | — |
| 443 | C | D | — |
| 444 | B | B | D |
| 445 | A | — | D |
| 446 | — | D | — |
| 447 | C | C | — |
| 448 | A | A | C |
| 449 | C | C | D |
| 450 | A | A | C |
| 451 | — | A | C |
| 452 | A | A | C |
| 453 | — | A | C |
| 454 | B | B | D |
| 455 | — | A | C |
| 456 | B | B | D |
| 457 | — | A | C |
| 458 | — | A | C |
| 459 | C | C | — |
| 460 | B | B | D |
| 461 | C | C | — |
| 462 | C | C | — |
| 463 | D | — | — |
| 464 | C | — | — |
| 465 | C | — | — |
| 466 | — | C | — |
| 467 | — | C | — |
| 468 | — | A | C |
| 469 | — | B | C |
| 470 | | A | D |
| 471 | | A | C |
| 472 | | A | D |
| 473 | | A | C |
| 474 | | A | C |
| 475 | | A | C |
| 476 | | A | C |
| 477 | | A | C |
| 478 | | A | D |
| 479 | | A | C |
| 480 | | A | C |
| 481 | | B | C |
| 482 | | A | C |
| 483 | | A | D |
| 484 | | C | D |
| 485 | | D | D |
| 486 | | D | D |
| 487 | | B | D |
| 488 | | C | |
| 489 | | C | D |
| 490 | | D | D |
| 491 | | C | D |
| 492 | | B | D |
| 493 | | B | C |
| 494 | | B | C |
| 495 | | B | D |
| 496 | | B | D |
| 497 | | A | D |
| 498 | | A | C |
| 499 | | A | C |
| 500 | | D | D |
| 501 | | C | D |
| 502 | | A | C |
| 503 | | B | C |
| 504 | | B | D |
| 505 | | B | D |
| 506 | | C | D |
| 507 | | C | D |
| 508 | | B | C |
| 509 | | A | C |
| 510 | | A | C |
| 511 | | B | D |
| 512 | | A | C |
| 513 | | B | D |
| 514 | | B | D |
| 515 | | C | D |
| 516 | | D | D |
| 517 | | A | D |
| 518 | | D | D |
| 519 | | A | C |
| 520 | | C | D |
| 521 | | C | D |
| 522 | | B | D |
| 523 | | A | C |
| 524 | | B | D |
| 525 | | B | D |
| 526 | | B | D |
| 527 | | B | D |
| 528 | | D | |
| 529 | | D | |
| 530 | | A | C |
| 531 | | A | C |
| 532 | | B | D |
| 533 | | B | D |
| 534 | | B | C |
| 535 | | B | D |
| 536 | | B | D |
| 537 | | B | D |
| 538 | | B | D |
| 539 | | A | D |
| 540 | | A | C |
| 541 | | A | |
| 542 | | A | C |
| 543 | | A | D |
| 544 | | A | C |
| 545 | | A | C |
| 546 | | A | C |
| 547 | | A | C |
| 548 | | A | D |
| 549 | | A | D |
| 550 | | B | D |
| 551 | | A | C |
| 552 | | A | C |
| 553 | | A | C |
| 554 | | B | D |
| 555 | | A | D |
| 556 | | A | D |
| 557 | A | | D |
| 558 | | A | C |
| 559 | | A | D |
| 560 | | A | D |
| 561 | | A | D |
| 562 | | A | C |
| 563 | | A | C |
| 564 | | A | C |
| 565 | | A | C |
| 566 | | A | D |
| 567 | | A | C |
| 568 | | B | |
| 569 | | A | C |
| 570 | | A | C |
| 571 | | A | D |
| 572 | | B | D |
| 573 | | A | D |
| 574 | | B | D |
| 575 | | A | C |
| 576 | | A | D |
| 577 | | A | C |
| 578 | | A | D |
| 579 | | A | C |
| 580 | | A | D |
| 581 | B | | D |
| 582 | B | | D |
| 583 | B | | D |
| 584 | | B | D |
| 585 | A | | D |
| 586 | D | | |
| 587 | | A | D |
| 588 | | C | D |
| 589 | | A | D |
| 590 | | A | D |
| 591 | | A | D |
| 592 | | A | D |
| 593 | | A | C |
| 594 | | B | D |
| 595 | | A | C |
| 596 | | A | C |
| 597 | | B | D |
| 598 | | B | D |
| 599 | | A | C |
| 600 | | B | D |
| 601 | | A | D |
| 602 | | A | D |
| 603 | | B | D |
| 604 | | A | D |
| 605 | | B | D |
| 606 | | B | D |
| 607 | | A | C |
| 608 | | B | D |
| 609 | | B | C |
| 610 | | A | C |
| 611 | | A | D |
| 612 | | A | C |
| 613 | | A | C |
| 614 | | B | C |
| 615 | | B | D |
| 616 | | B | D |
| 617 | | C | |
| 618 | | B | D |
| 619 | | A | D |
| 620 | | A | D |
| 621 | | A | C |
| 622 | | A | C |
| 623 | | A | C |
| 624 | | A | C |
| 625 | | A | C |
| 626 | | A | C |
| 627 | | B | D |
| 628 | | A | C |
| 629 | | A | D |
| 630 | | A | C |
| 631 | | A | C |
| 632 | | A | C |
| 633 | | A | B |
| 634 | | B | D |
| 635 | | B | D |
| 636 | | A | C |
| 637 | | A | C |
| 638 | | A | C |
| 639 | | A | C |
| 640 | | B | D |
| 641 | | A | C |
| 642 | | A | D |
| 643 | | A | C |
| 644 | | A | C |
| 645 | | A | D |
| 646 | | C | D |
| 647 | | A | C |
| 648 | | A | D |
| 649 | | A | C |
| 650 | | A | C |
| 651 | | A | C |
| 652 | | A | C |
| 653 | | A | C |
| 654 | | A | D |
| 655 | | A | D |
| 656 | | A | C |
| 657 | | A | D |
| 658 | | A | C |
| 659 | | B | D |
| 660 | | B | D |
| 661 | | B | D |
| 662 | | B | D |
| 663 | | A | |
| 664 | | C | D |
| 665 | | A | C |
| 666 | | B | D |
| 667 | | B | D |
| 668 | | A | D |
| 669 | | A | D |
| 670 | | B | C |
| 671 | | A | C |
| 672 | | A | C |
| 673 | | B | D |
| 674 | | A | C |
| 675 | | A | C |
| 676 | | A | D |
| 677 | | B | D |
| 678 | | B | |
| 679 | | B | D |
| 680 | | A | C |
| 681 | | A | C |
| 682 | | A | D |
| 683 | | A | D |
| 684 | | B | D |
| 685 | | B | D |
| 686 | | A | |
| 687 | | A | D |
| 688 | | B | D |
| 689 | | B | |
| 690 | | | |
| 691 | | | |
| 692 | | | |
| 693 | | | D |
| 694 | | A | C |
| 695 | | A | D |
| 696 | | A | B |
| 697 | A | A | C |
| 698 | A | | C |
| 699 | A | B | C |
| 700 | | B | D |
| 701 | | B | D |
| 702 | | A | D |
| 703 | | A | C |
| 704 | | B | D |
| 705 | A | | C |
| 706 | | A | C |
| 707 | | A | B |
| 708 | A | | C |
| 709 | | A | D |
| 710 | | A | C |
| 711 | | A | C |
| 712 | | B | D |
| 713 | | A | C |
| 714 | | B | D |
| 715 | | | D |
| 716 | | A | D |
| 717 | | A | C |
| 718 | | A | D |
| 719 | C | D | D |
| 720 | C | C | |
| 721 | | B | D |
| 722 | | A | B |
| 723 | | B | D |
| 724 | | A | B |
| 725 | | A | D |
| 726 | | A | B |
| 727 | | A | B |
| 728 | | A | C |
| 729 | | A | C |
| 730 | | A | D |
| 731 | | A | C |
| 732 | | B | D |
| 733 | | B | C |
| 734 | | A | C |
| 735 | | A | D |
| 736 | | A | |
| 737 | | A | C |
| 738 | | B | D |
| 739 | | B | D |
| 740 | | A | D |
| 741 | | B | D |
| 742 | | B | D |
| 743 | | A | |
| 744 | | A | D |
| 745 | | A | |
| 746 | | A | D |
| 747 | | B | |
| 748 | | A | D |
| 749 | | B | D |
| 750 | | B | D |
| 751 | | B | D |
| 752 | | B | D |
| 753 | | B | D |
| 754 | | B | D |
| 755 | | A | |
| 756 | | A | D |
| 757 | | A | C |
| 758 | | A | C |
| 759 | | A | D |
| 760 | | A | |
| 761 | | B | D |
| 762 | | B | D |
| 763 | | B | D |
| 764 | | B | D |
| 765 | | B | D |
| 766 | | B | D |
| 767 | | | D |
| 768 | | A | C |
| 769 | | | D |
| 770 | | | D |
| 771 | | | D |
| 772 | | | D |
| 773 | | A | C |
| 774 | | A | |
| 775 | | | D |
| 776 | | | D |
| 777 | | B | D |
| 778 | | A | C |
| 779 | | A | C |
| 780 | | B | D |
| 781 | | B | D |
| 782 | | A | C |
| 783 | | B | D |
| 784 | | A | C |
| 785 | | B | C |
| 786 | | B | D |
| 787 | | A | D |
| 788 | | A | D |
| 789 | | A | C |
| 790 | | A | C |
| 791 | | A | D |
| 792 | | B | D |
| 793 | | B | D |
| 794 | | A | C |
| 795 | | A | C |
| 796 | | A | C |
| 797 | | A | D |
| 798 | | A | D |
| 799 | | A | D |
| 800 | | A | D |
| 801 | | B | D |
| 802 | | A | D |
| 803 | | B | D |
| 804 | | A | C |
| 805 | | A | C |
| 806 | | B | D |
| 807 | | A | D |
| 808 | | A | C |
| 809 | | A | C |
| 810 | | A | C |
| 811 | | A | D |
| 812 | | B | D |
| 813 | | A | D |
| 814 | | B | D |
| 815 | | B | D |
| 816 | | A | C |
| 817 | | A | C |
| 818 | | A | D |
| 819 | | A | C |
| 820 | | B | D |
| 821 | | B | D |
| 822 | | C | D |
| 823 | | C | D |
| 824 | | A | D |
| 825 | | B | D |
| 826 | | | C |
| 827 | | B | D |
| 828 | | B | D |
| 829 | | B | D |
| 830 | | B | D |
| 831 | | A | D |
| 832 | | B | D |
| 833 | | A | C |
| 834 | | A | B |
| 835 | | B | C |
| 836 | | B | B |
| 837 | | B | C |
| 838 | | A | B |
| 839 | | B | C |
| 840 | | A | B |
| 841 | | A | D |
| 842 | | A | C |
| 843 | | A | D |
| 844 | | A | C |
| 845 | | B | D |
| 846 | | B | D |
| 847 | | B | D |
| 848 | | B | D |
| 849 | | B | D |
| 850 | | B | C |
| 851 | | A | C |
| 852 | | B | D |
| 853 | | B | D |
| 854 | | A | C |
| 855 | | B | D |
| 856 | | B | D |
| 857 | | B | D |
| 858 | | B | D |
| 859 | | A | D |
| 860 | | A | D |
| 861 | | A | C |
| 862 | | A | D |
| 863 | | B | D |
| 864 | | A | D |
| 865 | | B | D |
| 866 | | A | D |
| 867 | | A | D |
| 868 | | A | D |
| 869 | | B | D |
| 870 | | A | D |
| 871 | | B | D |
| 872 | | B | D |
| 873 | | B | D |
| 874 | | B | D |
| 875 | | B | D |
| 876 | | A | D |
| 877 | | A | |
| 878 | | A | C |
| 879 | | B | D |
| 880 | | A | D |
| 881 | | B | D |
| 882 | | A | C |
| 883 | | A | D |
| 884 | | A | D |
| 885 | | A | D |
| 886 | | A | D |
| 887 | | A | D |
| 888 | | A | D |
| 889 | | A | |
| 890 | | A | D |
| 891 | | C | D |
| 892 | | A | C |
| 893 | | A | C |
| 894 | | A | C |
| 895 | | | D |
| 896 | | B | D |
| 897 | | A | |
| 898 | | A | D |
| 899 | | A | |
| 900 | | A | D |
| 901 | | A | C |
| 902 | | A | D |
| 903 | | B | D |
| 904 | | A | C |
| 905 | | A | D |
| 906 | | A | |
| 907 | | A | D |
| 908 | | A | C |
| 909 | | A | C |
| 910 | | A | D |
| 911 | | A | D |
| 912 | | A | D |
| 913 | | A | |
| 914 | | A | C |
| 915 | | A | D |
| 916 | | A | C |
| 917 | | A | C |
| 918 | | A | D |
| 919 | | A | D |
| 920 | | A | D |
| 921 | | A | C |
| 922 | | B | D |
| 923 | | | D |
| 924 | | A | D |
| 925 | | A | D |
| 926 | | A | D |
| 927 | | A | C |
| 928 | | A | D |
| 929 | | A | C |
| 930 | | A | C |
| 931 | | A | C |
| 932 | | | C |
| 933 | | A | C |
| 934 | | A | C |
| 935 | | A | D |
| 936 | | A | C |
| 937 | | A | |
| 938 | | A | |
| 939 | | A | C |
| 940 | | A | C |
| 941 | | A | |
| 942 | | A | C |
| 943 | | A | B |
| 944 | | A | C |
| 945 | | A | D |
| 946 | | B | C |
| 947 | | A | C |
| 948 | | A | C |
| 949 | | A | D |
| 950 | | A | D |
| 951 | | A | C |
| 952 | | A | C |
| 953 | | A | C |
| 954 | | A | C |
| 955 | | A | C |
| 956 | | A | C |
| 957 | | A | C |
| 958 | | A | C |
| 959 | | A | C |
| 960 | | A | D |
| 961 | | A | C |
| 962 | | A | D |
| 963 | | A | C |
| 964 | | B | D |
| 965 | | B | D |
| 966 | | A | C |
| 967 | | A | C |
| 968 | | A | C |
| 969 | | A | D |
| 970 | | A | C |
| 971 | | A | C |
| 972 | | A | C |
| 973 | | B | D |
| 974 | | B | D |
| 975 | | A | C |
| 976 | | A | C |
| 977 | | A | D |
| 978 | | A | D |
| 979 | | A | |
| 980 | | B | D |
| 981 | | A | D |
| 982 | | A | D |
| 983 | | A | D |
| 984 | | A | C |
| 985 | | A | C |
| 986 | | A | C |
| 987 | | A | D |
| 988 | | B | D |
| 989 | | B | D |
| 990 | | A | D |
| 991 | | C | D |
| 992 | | A | C |
| 993 | | A | C |
| 994 | | B | D |
| 995 | | B | C |
| 996 | | B | D |
| 997 | | B | D |
| 998 | | A | C |
| 999 | | A | C |
| 1000 | | A | C |
| 1001 | | B | D |
| 1002 | | C | D |
| 1003 | | B | D |
| 1004 | | A | C |
| 1005 | | B | D |
| 1006 | | B | C |
| 1007 | | | D |
| 1008 | | A | C |
| 1009 | | A | D |
| 1010 | | A | |
| 1011 | | A | D |
| 1012 | | A | D |
| 1013 | | A | C |
| 1014 | | A | C |
| 1015 | | A | C |
| 1016 | | A | C |
| 1017 | | A | C |
| 1018 | | A | D |
| 1019 | | A | C |
| 1020 | | B | D |
| 1021 | | B | D |
| 1022 | | B | |
| 1023 | | A | C |
| 1024 | | B | D |
| 1025 | | B | D |
| 1026 | | B | D |
| 1027 | | A | C |
| 1028 | | B | D |
| 1029 | | A | B |
| 1030 | | A | C |
| 1031 | | | D |
| 1032 | | | D |
| 1033 | | A | C |
| 1034 | | B | D |
| 1035 | | B | D |
| 1036 | | A | D |
| 1037 | | A | D |
| 1038 | | A | C |
| 1039 | | A | D |
| 1040 | B | | D |
| 1041 | | C | D |
| 1042 | | A | C |
| 1043 | | B | D |
| 1044 | | A | C |
| 1045 | | A | C |
| 1046 | | C | D |
| 1047 | | C | |
| 1048 | | C | |
| 1049 | | C | D |
| 1050 | | B | D |
| 1051 | | A | C |
| 1052 | | A | C |
| 1053 | | A | C |
| 1054 | B | B | D |
| 1055 | | C | |
| 1056 | C | D | |
| 1057 | B | | D |

A = X < 10 nM;
B = 10 nM < X < 100 nM;
C = 100 nM < X < 1000 nM;
D = 1000 nM < X
^{a}Data Obtained using Integrin Adhesion Assay: Protocol 4
^{b}Data Obtained using Integrin Adhesion Assay: Protocol 5

We claim:

1. A compound of Formula (I): 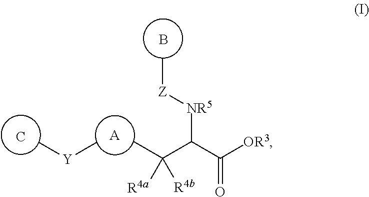
or a pharmaceutically acceptable salt thereof, wherein:
A is selected from: 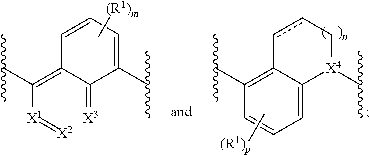
X¹, X², X³, and X⁴ are each independently selected from N and C(R²), wherein at least one of X¹, X², and X³ is C(R²); [symbol]
represents a single bond or a double bond;
m is 0, 1, 2, or 3;
n is 0, 1, or 2;
p is 0, 1, 2, or 3;
each R¹ is independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂ (R¹¹), —S(O)R¹¹, —S(O)₂R¹¹, —S(O)₂N(R¹¹)₂, —NO₂, and —CN;
C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN;
C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
each R₂ is independently selected from:
hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂ (R¹²), —S(O)R¹², —S(O)₂R¹², —S(O)₂N(R¹²)₂, —NO₂, and —CN;
C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
R^{4a} and R^{4b} are each independently selected from:
hydrogen, halogen, —OR¹³, —SR¹³, —N(R¹³)₂, —C(O)R¹³, —NO₂, —CN, and C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Y is a bond, —O—, —N(R¹⁴)— or —C(R^{14a})(R^{14b})—;
Z is selected from:
a bond, —C(O)—, —C(R¹⁵)₂—, —C(O)C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—;
wherein q is selected from 1, 2, and 3;
B is selected from C₃₋₁₂ carbocycle and 3- to 12-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl, C₂₋₆ alkenyl, and C₂₋₆ alkynyl, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN;
C is selected from C₃₋₁₂ carbocycle and 3- to 12-membered heterocycle, each of which is optionally substituted with one or more substituents independently selected from:
halogen, —OR¹⁸, —OR^{18A}, —SR¹⁸, —N(R¹⁸)₂, —C(O)R¹⁸, —C(O)R^{18A}, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN; and
C₃₋₁₂ carbocycle and 3- to 12-membered heterocycle any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, —NR^{18A}, —N₃, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from halogen, —OR¹⁸, —SR¹⁸, —N(R¹⁸)₂, —C(O)R¹⁸, —NO₂, ═O, ═S, —NR¹⁸, —N₃, —CN; and C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, ═O, and CN;
R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN;
R¹³ is independently selected at each occurrence from hydrogen and C₁₋₆ alkyl;
R^{14a} and R^{14b} are each independently selected from:
hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN; or
R^{14a} and R^{14b} are taken together to form a C₃₋₆ carbocycle or a 3- to 6-membered heterocycle, each of which is optionally substituted with one or more substituents independently selected from halogen, —OH, —NH₂, C₁₋₆ alkyl, —C₁₋₆ haloalkyl, —O—C₁₋₆ alkyl, —O—C₁₋₆ haloalkyl, ═O, and —CN;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R¹⁵ are taken together to form a C₃₋₆ carbocycle;
R¹⁶ is selected at each occurrence from hydrogen and C₁₋₆ alkyl;
R¹⁷ is independently selected at each occurrence from:
hydrogen;
C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
C₃₋₁₀ carbocycle and 3- to 10-membered heterocycle, each of which is optionally substituted with one or more substituents independently selected from: halogen, C₁₋₆ alkyl, C₁₋₆ haloalkyl —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂ (R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and
R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl; and
R^{18A} is selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, —CN, C₃₋₆ carbocycle and 3- to 6-membered heterocycle.

2. The compound or salt of claim 1, wherein B is 3- to 12-membered heterocycle optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl, C₂₋₆ alkenyl, and C₂₋₆ alkynyl, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN.

3. The compound or salt of claim 2, wherein B is selected from 3- to 12-membered heterocycle optionally substituted with one or more substituents independently selected from:
halogen, —OR¹⁷, —SR¹⁷, —N(R¹⁷)₂, —C(O)R¹⁷, —C(O)OR¹⁷, —NO₂, —CN;
C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, —CN, C₃-10 carbocycle and 3- to 10-membered heterocycle, wherein the C₃₋₁₀ carbocycle and 3- to 10-membered heterocycle are each optionally substituted with one or more substituents independently selected from: halogen, —OR¹⁷, —SR¹⁷, —N(R¹⁷)₂, —C(O)R¹⁷, —C(O)OR¹⁷, —NO₂, —CN; and
C₃₋₁₀ carbocycle and 3- to 10-membered heterocycle, each of which is optionally substituted with one or more substituents independently selected from: halogen, —OR¹⁷, —SR¹⁷, —N(R¹⁷)₂, —C(O)R¹⁷, —C(O)OR¹⁷, —NO₂, —CN; and C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —NO₂, and —CN.

4. The compound or salt of claim 3, wherein B is a 6- to 12-membered bicyclic heterocycle selected from 6- to 12-membered fused heterocycle, 6- to 12-membered spirocyclic heterocycle, and 6- to 12-membered bridged heterocycle, each of which is optionally substituted with one or more substituents independently selected from halogen, —OR¹⁷, —SR¹⁷, —N(R¹⁷)₂, —C(O)R¹⁷, —C(O)OR¹⁷, —NO₂, —CN; and C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, and —CN.

5. The compound or salt of claim 4, wherein B is 6- to 12-membered spirocyclic heterocycle optionally substituted with one or more substituents independently selected from halogen, —OR¹⁷, —SR¹⁷, —N(R¹⁷)₂, —C(O)R¹⁷, —C(O)OR¹⁷, —NO₂, —CN; and C₁₋₆ alkyl, optionally substituted with one or more substituents independently selected from: halogen, —OR¹⁷, —SR¹⁷, —N(R¹⁷)₂, —C(O)R¹⁷, —C(O)OR¹⁷, —NO₂, and —CN.

6. The compound or salt of claim 5, wherein B is

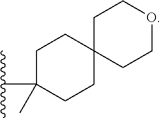 [SMILES: *C1(C)CCC2(CCOCC2)CC1]

7. The compound or salt of claim 1, wherein C is selected from C₃₋₁₀ carbocycle and 3- to 10-membered heterocycle, optionally substituted with —OR^{18A} or —C(O)R^{18A}, and R^{18A} is selected from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, and —CN; and
C₃₋₆ carbocycle and 3- to 6-membered heterocycle optionally substituted with one or more substituents independently selected from: halogen, —OH, C₁₋₆ alkyl, C₁₋₆ haloalkyl, —O—C₁₋₆ alkyl, —O—C₁₋₆ haloalkyl, —NH₂, —NO₂, ═O, —CN, C₃₋₆ carbocycle and 3- to 6-membered heterocycle.

8. The compound or salt of claim 7, wherein C is selected from

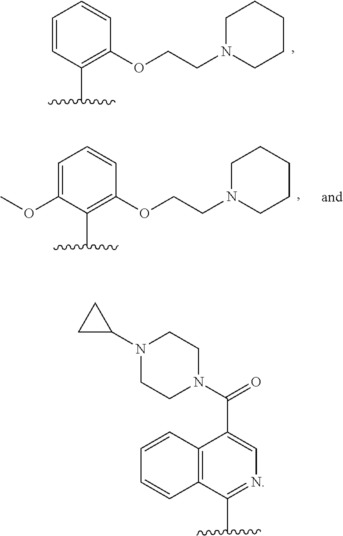

9. The compound or salt of claim 1, wherein C is C₃₋₁₂ carbocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN; and
C₃₋₁₂ carbocycle and 3- to 12-membered heterocycle, any of which is optionally substituted with one or more substituents independently selected from:
halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
halogen, —OR¹⁸, —SR¹⁸, —N(R¹⁸)₂, —C(O)R¹⁸, —NO₂, ═O, ═S, ═NR¹⁸, —N₃, —CN; and
C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, and —CN.

10. The compound or salt of claim 9, wherein C is C₃₋₁₀ carbocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN;
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
halogen, —OR¹⁸, —SR¹⁸, —N(R¹⁸)₂, —C(O)R¹⁸, —NO₂, and —CN; and
C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₃₋₆ carbocycle and 3- to 6-membered heterocycle, wherein C₃₋₆ carbocycle and 3- to 6-membered heterocycle, are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN.

11. The compound or salt of claim 10, wherein C is phenyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

12. The compound or salt of claim 11, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from: halogen, —OR¹⁸, —N(R¹⁸)₂, —NO₂, and —CN; and 3- to 12-membered saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

13. The compound or salt of claim 12, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from: halogen, —OR¹⁸, —N(R¹⁸)₂, —NO₂, and —CN; and 3- to 7-membered heterocycloalkyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

14. The compound or salt of claim 13, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, —CN; and
azetidinyl, piperidinyl, piperazinyl, and azapanyl, each of which is optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and each of R¹⁸ and R^{18A} are independently selected at each occurrence from hydrogen, halogen, C₁₋₆ alkyl, and C₁₋₆ haloalkyl.

15. The compound or salt of claim 14, wherein C is selected from:

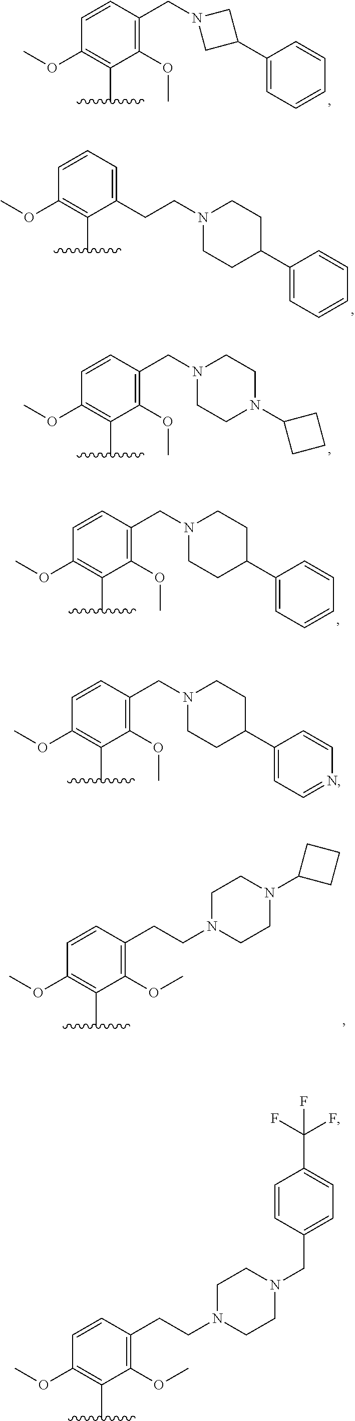

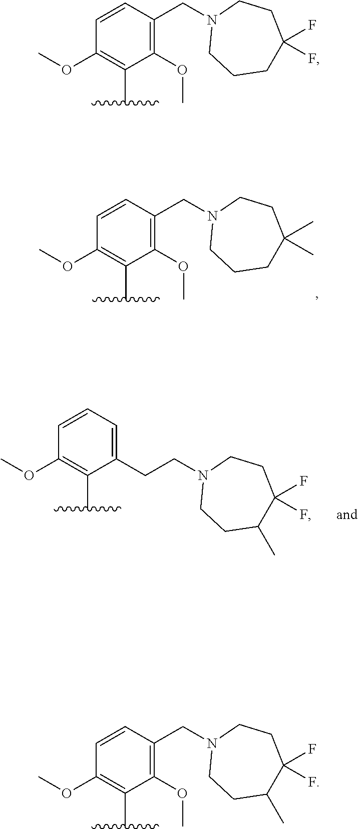

16. The compound or salt of claim 12, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, and —CN; and
8- to 12-membered spirocyclic saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle.

17. The compound or salt of claim 16, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, and —CN; and
6-azaspiro[2.5]octanyl, 2-oxa-8-azaspiro[4.5]decanyl, 3-azaspiro[5.5]undecanyl, and 2,9-diazaspiro[5.5]undecanyl, each of which is optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

18. The compound or salt of claim 17, wherein C is selected from:

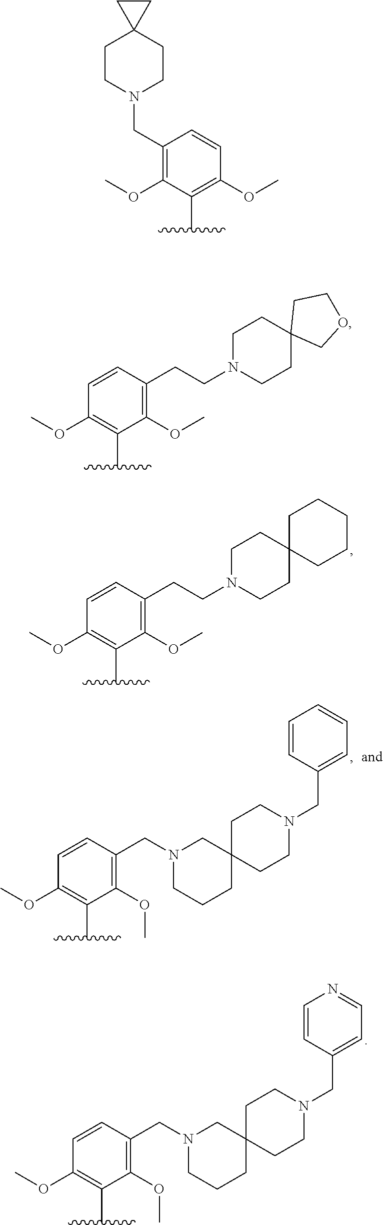

19. The compound or salt of claim 12, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, and —CN; and
8- to 10-membered bridged saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle.

20. The compound or salt of claim 16, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, and —CN; and
8-oxa-3-azabicyclo[3.2.1]octanyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

21. The compound or salt of claim 20, wherein C is selected from:

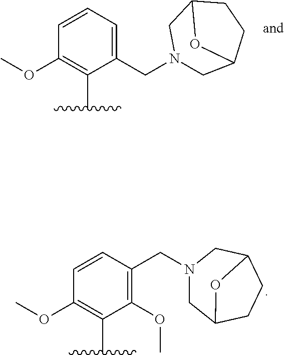 [SMILES: *c1c(CN2CC3CCC(C2)O3)cccc1OC ; *c1c(OC)ccc(CN2CC3CCC(C2)O3)c1OC]

22. The compound or salt of claim 12, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, and —CN; and
6- to 10-membered fused saturated heterocycle optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle.

23. The compound or salt of claim 16, wherein C is phenyl optionally substituted with one or more substituents independently selected from:
halogen, —OR¹⁸, —N(R¹⁸)₂, —NO₂, and —CN; and
C₁₋₆ alkyl optionally substituted with one or more substituents independently selected from:
halogen, —OR¹⁸, —N(R¹⁸)₂, —NO₂, and —CN; and
octahydro-1H-isoindolyl optionally substituted with one or more substituents independently selected from: halogen, —OR^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —NO₂, —CN, C₁₋₆ alkyl, C₁₋₆ haloalkyl, C₃₋₆ carbocycle, and 3- to 6-membered heterocycle; and each of R¹⁸ and R^{18A} are independently selected at each occurrence from hydrogen, halogen, C₁₋₆ alkyl, and C₁₋₆ haloalkyl.

24. The compound or salt of claim 23, wherein C is

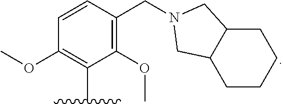 [SMILES: *c1c(OC)ccc(CN2CC3CCCCC3C2)c1OC]

25. The compound or salt of claim 9, wherein C is C₆₋₁₂ carbocycle optionally substituted with one or more substituents independently selected from C₃₋₆ carbocycle and 3- to 6-membered heterocycle, each of which is optionally substituted with one or more substituents independently selected from:
halogen, —OR¹⁸, —SR¹⁸, —N(R¹⁸)₂, —C(O)R¹⁸, —NO₂, ═O, ═S, ═NR¹⁸, —N₃, —CN; and
C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, and —CN.

26. The compound or salt of claim 25, wherein C is C₈₋₁₂ fused carbocycle optionally substituted with one or more substituents independently selected from 5- to 6-membered heteroaryl optionally substituted with one or more substituents independently selected from:
halogen, —OR¹⁸, —SR¹⁸, —N(R¹⁸)₂, —NO₂, —CN; and
C₁₋₆ alkyl optionally substituted with one or more substituents independently selected from halogen, —OR^{18A}, —N(R^{18A})₂, —NO₂, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —N(R^{18A})₂, —NO₂, —CN; and each of R¹⁸ and R^{18A} are independently selected at each occurrence from hydrogen, halogen, C₁₋₆ alkyl, and C₁₋₆ haloalkyl.

27. The compound or salt of claim 26, wherein C is

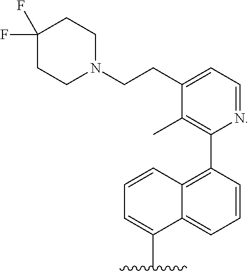 [SMILES: *c1cccc2c(-c3nccc(CCN4CCC(F)(F)CC4)c3C)cccc12]

28. The compound or salt of claim 1, wherein C is 3- to 12-membered heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, —NR¹⁸, —N₃, and —CN; and
C₃₋₁₂ carbocycle and 3- to 12-membered heterocycle, any of which is optionally substituted with one or more substituents independently selected from:
halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₁₀ alkyl, C₃₋₁₀ carbocycle and 3- to 10-membered heterocycle, wherein C₁₋₁₀ alkyl, C₃₋₁₀ carbocycle and 3- to 10-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
halogen, —OR¹⁸, —SR¹⁸, —N(R¹⁸)₂, —C(O)R¹⁸, —NO₂, ═O, ═S, ═NR¹⁸, —N₃, —CN; and
C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, —CN, C₃₋₆ carbocycle and 3- to 6-membered heterocycle, wherein the C₃₋₆ carbocycle and 3- to 6-membered heterocycle are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —N(R^{18A})C(O)OR^{18A}, —N(R^{18A})C(O)N(R^{18A})₂, —N(R^{18A})C(S)N(R^{18A})₂, —N(R^{18A})S(O)₂ (R^{18A}), —S(O)R^{18A}, —S(O)₂R^{18A}, —S(O)₂N(R^{18A})₂, —NO₂, ═O, ═S, ═NR^{18A}, —N₃, and —CN.

29. The compound or salt of claim 28, wherein C is pyridinyl, optionally substituted with one or more substituents independently selected from:
halogen, —OR¹⁸, —N(R¹⁸)₂, —C(O)R¹⁸, —NO₂, and —CN;
C₁₋₆ alkyl, optionally substituted with one or more substituents independently selected from:
halogen, —OR¹⁸, —N(R¹⁸)₂, —C(O)R¹⁸, —NO₂, and —CN; and
C₃₋₁₂ carbocycle and 3- to 12-membered heterocycle, any of which is optionally substituted with one or more substituents independently selected from:
halogen, —OR^{18A}, —N(R^{18A})₂, —C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —N(R^{18A})₂, —C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
each of R¹⁸ and R^{18A} are independently selected at each occurrence from hydrogen, halogen, C₁₋₆ alkyl, and C₁₋₆ haloalkyl.

30. The compound or salt of claim 29, wherein C is selected from:

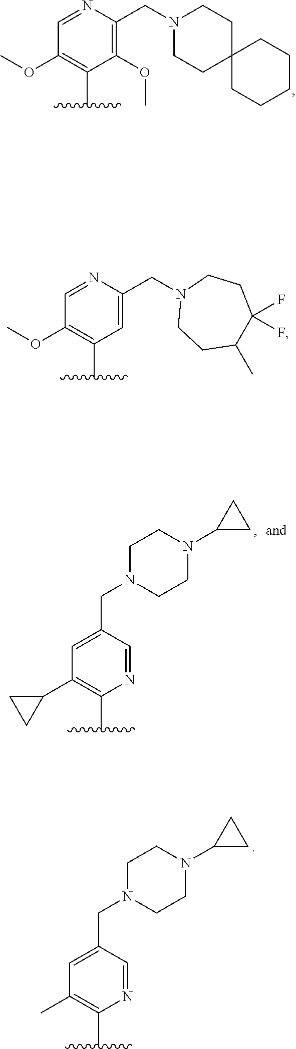 [SMILES: *c1c(OC)cnc(CN2CCC3(CCCCC3)CC2)c1OC ; *c1cc(CN2CCC(C)C(F)(F)CC2)ncc1OC ; *c1ncc(CN2CCN(C3CC3)CC2)cc1C1CC1 ; *c1ncc(CN2CCN(C3CC3)CC2)cc1C]

31. The compound or salt of claim 28, wherein C is selected from a 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN;
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle; and
C₃₋₆ carbocycle and 3- to 6-membered heterocycle, each of which is optionally substituted with one or more substituents independently selected from:
halogen, —OR¹⁸, —SR¹⁸, —N(R¹⁸)₂, —C(O)R¹⁸, —NO₂, and —CN; and
C₁₋₆ alkyl optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₃₋₆ carbocycle and 3- to 6-membered heterocycle, wherein C₃₋₆ carbocycle and 3- to 6-membered heterocycle, are each optionally substituted with one or more substituents independently selected from halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN.

32. The compound or salt of claim 31, wherein C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₃₋₁₂ carbocycle and 3- to 12-membered heterocycle, any of which is optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

33. The compound or salt of claim 32, wherein C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
3- to 8-membered monocyclic heterocycloalkyl optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

34. The compound or salt of claim 32, wherein C is 8- to 12-membered bicyclic heterocycle optionally substituted with one or more substituents independently selected from:
halogen, —OR¹⁸, —N(R¹⁸)₂, —C(O)R¹⁸, ═O, —NO₂, and —CN; and
C₁₋₆ alkyl optionally substituted with one or more substituents independently selected from:
halogen, —OR¹⁸, —N(R¹⁸)₂, —C(O)R¹⁸, —NO₂, and —CN; and
3- to 8-membered monocyclic heterocycloalkyl optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle.

35. The compound or salt of claim 34, wherein C is selected from

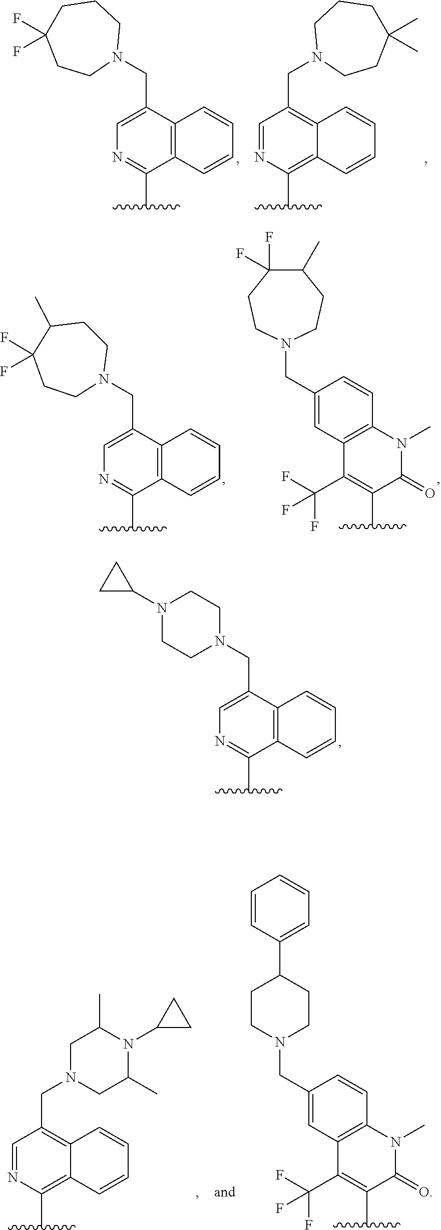

36. The compound or salt of claim 32, wherein C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
3- to 12-membered bicyclic heterocycle optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

37. The compound or salt of claim 36, wherein C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
6- to 12-membered spirocyclic heterocycle optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

38. The compound or salt of claim 37, wherein C is 8- to 12-membered bicyclic heterocycle optionally substituted with one or more substituents independently selected from:
halogen, —OR¹⁸, —N(R¹⁸)₂, —C(O)R¹⁸, ═O, —NO₂, and —CN; and
C₁₋₆ alkyl optionally substituted with one or more substituents independently selected from:
halogen, —OR¹⁸, —N(R¹⁸)₂, —C(O)R¹⁸, —NO₂, and —CN; and
6- to 12-membered spirocyclic heterocycle optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle.

39. The compound or salt of claim 38, wherein C is selected from

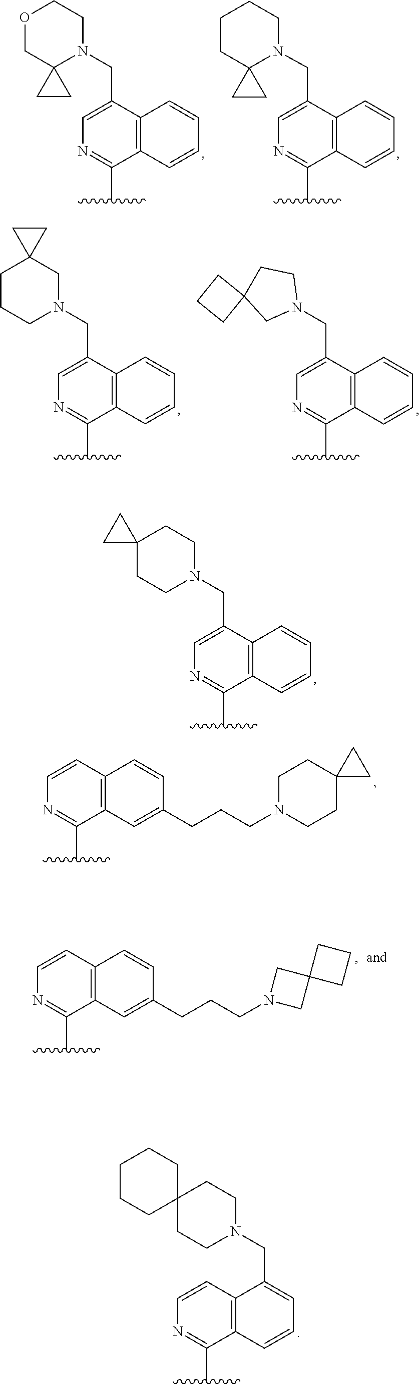

40. The compound or salt of claim 36, wherein C is 8- to 12-membered bicyclic heterocycle optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, ═O, —NO₂, and —CN; and
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —NO₂, and —CN; and
6- to 12-membered bridged heterocycle optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle, are each optionally substituted with one or more substituents independently selected from: halogen, —OR^{18A}, —SR^{18A}, —N(R^{18A})₂, —C(O)R^{18A}, —C(O)OR^{18A}, —OC(O)R^{18A}, —OC(O)N(R^{18A})₂, —C(O)N(R^{18A})₂, —N(R^{18A})C(O)R^{18A}, —NO₂, —CN, C₁₋₆ alkyl, C₁₋₆ haloalkyl, C₃₋₆ carbocycle, —C₃₋₆ carbocycle-(C₁₋₆ haloalkyl), and 3- to 6-membered heterocycle.

41. The compound or salt of claim 39, wherein C is 8- to 12-membered bicyclic heterocycle optionally substituted with one or more substituents independently selected from:
halogen, —OR¹⁸, —N(R¹⁸)₂, —C(O)R¹⁸, ═O, —NO₂, and —CN; and
C₁₋₆ alkyl optionally substituted with one or more substituents independently selected from:
halogen, —OR¹⁸, —N(R¹⁸)₂, —C(O)R¹⁸, —NO₂, and —CN; and
6- to 12-membered bridged heterocycle optionally substituted with one or more substituents independently selected from: halogen, —OR^{18A}, —N(R^{18A})₂, —C(O)R^{18A}, —NO₂, —CN, C₁₋₆ alkyl, C₃₋₆ carbocycle and 3- to 6-membered heterocycle.

42. The compound or salt of claim 41, wherein C is selected from

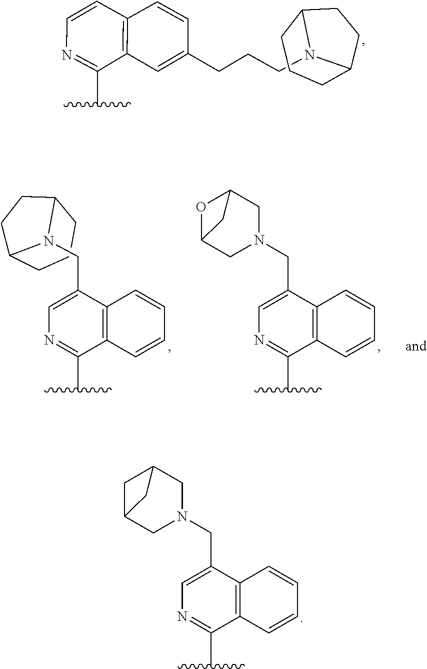 [SMILES: *c1nccc2ccc(CCCN3C4CCCC3CC4)cc12 ; *c1ncc(CN2C(CC)CCC2CC)c2ccccc12 ; *c1ncc(CN2CC3CC(C2)O3)c2ccccc12 ; *c1ncc(CN2CC3CC(C3)C2)c2ccccc12]

43. The compound or salt of claim 1, wherein Formula (I) is represented by the structure of Formula (I-a): 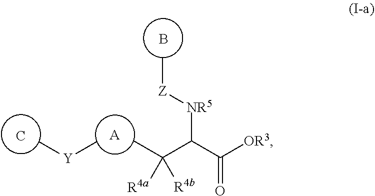
or a pharmaceutically acceptable salt thereof, wherein:
A is selected from: 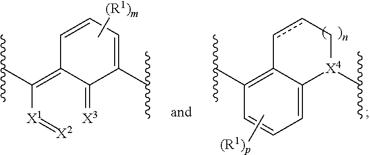
X¹, X², X³, and X⁴ are each independently selected from N and C(R²), wherein at least one of X¹, X², and X³ is C(R²); [symbol]
represents a single bond or a double bond;
m is 0, 1, 2, or 3;
n is 0, 1, or 2;
p is 0, 1, 2, or 3;
each R¹ is independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)C(S)N(R¹¹)₂, —N(R¹¹)S(O)₂ (R¹¹), —S(O)R¹¹, —S(O)₂R¹¹, —S(O)₂N(R¹¹)₂, —NO₂, and —CN;
C₁₋₆ alkyl, C₂₋₆ alkenyl, and C₂₋₆ alkynyl, each of which is optionally substituted by one or more substituents independently selected from halogen, —OR¹¹, —SR¹¹, —N(R¹¹)₂, ═O, ═S, and —CN;
C₃₋₆ carbocycle and 3- to 6-membered heterocycle, each of which is optionally substituted with one or more substituents independently selected from halogen, C₁₋₄ alkyl, C₁₋₄ haloalkyl, —OR¹¹, —SR¹¹, —N(R¹¹)₂, ═O, ═S, and —CN;
each R₂ is independently selected from:
hydrogen, halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)C(S)N(R¹²)₂, —N(R¹²)S(O)₂ (R¹²), —S(O)R¹², —S(O)₂R¹², —S(O)₂N(R¹²)₂, —NO₂, and —CN;
C₁₋₆ alkyl, C₂₋₆ alkenyl, and C₂₋₆ alkynyl, each of which is optionally substituted by one or more substituents independently selected from halogen, —OR¹², —SR¹², —N(R¹²)₂, ═O, ═S, and —CN; and
C₃₋₆ carbocycle and 3- to 6-membered heterocycle, each of which is optionally substituted with one or more substituents independently selected from: halogen, C₁₋₄ alkyl, C₁₋₄ haloalkyl, —OR¹², —SR¹², —N(R¹²)₂, ═O, ═S, and —CN;
R³ is hydrogen or C₁₋₆ alkyl;
R^{4a} and R^{4b} are each independently selected from:
hydrogen, halogen, —OR¹³, —SR¹³, —N(R¹³)₂, —C(O)R¹³, —NO₂, —CN, and C₁₋₆ alkyl, optionally substituted with one or more substituents independently selected from halogen, —OR¹³, —SR¹³, —N(R¹³)₂, —C(O)R¹³, —NO₂, and —CN;
R⁵ is hydrogen or C₁₋₆ alkyl;
Y is a bond, —O—, —N(R¹⁴)— or —C(R^{14a})(R^{14b})—;
Z is selected from:
a bond, —C(O)—, —C(R¹⁵)₂—, —C(O)C(R¹⁵)₂—, —C(O) [C(R¹⁵)₂]_{q}S—, —C(═N—CN)—, —C(O)O—, —C(O)N(R¹⁶)—, —C(S)—, —C(S)N(R¹⁶)—, —S(O)—, and —S(O)₂—;
wherein q is selected from 1, 2, and 3;
B is selected from C₃₋₁₀ carbocycle and 3- to 10-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl, C₂₋₆ alkenyl, and C₂₋₆ alkynyl, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₃₋₁₀ carbocycle and 3- to 10-membered heterocycle, each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle, each of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN;
C₁₋₆ alkyl optionally substituted with one or more substituents independently selected from: halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, ═S, ═NR¹⁷, —N₃, and —CN; and
C₃₋₁₀ carbocycle and 3- to 10-membered heterocycle each of which is optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —NO₂, ═O, ═S, ═NR¹⁷, —N₃, and —CN;
C is selected from C₅₋₁₀ carbocycle and 5- to 10-membered heterocycle, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN;
C₁₋₆ alkyl, C₂₋₆ alkenyl, and C₂₋₆ alkynyl, each of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)R¹⁸, —S(O)₂R¹⁸, —S(O)₂N(R¹⁸)₂, —NO₂, ═O, ═S, ═NR¹⁸, —N₃, and —CN; and
C₃₋₆ carbocycle and 3- to 6-membered heterocycle any of which is optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —NO₂, ═O, ═S, ═NR¹⁸, —N₃, and —CN;
R¹¹ and R¹² are each independently selected at each occurrence from hydrogen and C₁₋₆ alkyl, wherein the C₁₋₆ alkyl is optionally substituted with one or more substituents independently selected from: halogen, —OH, —NH₂, —O—C₁₋₆ alkyl, —O—C₁₋₆ haloalkyl, ═O, and CN;
R¹⁴ is selected from hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN;
R¹³ is independently selected at each occurrence from hydrogen and C₁₋₆ alkyl;
R^{14a} and R^{14b} are each independently selected from:
hydrogen, C₁₋₆ alkyl, and C₃₋₆ carbocycle, wherein each of the C₁₋₆ alkyl and C₃₋₆ carbocycle is optionally substituted with one or more substituents independently selected from halogen, —OH, —NH₂, —O—C₁₋₆ alkyl, —O—C₁₋₆ haloalkyl, ═O, and —CN; or
R^{14a} and R^{14b} are taken together to form a C₃₋₆ carbocycle or a 3- to 6-membered heterocycle, each of which is optionally substituted with one or more substituents independently selected from halogen, —OH, —NH₂, C₁₋₆ alkyl, —C₁₋₆ haloalkyl, —O—C₁₋₆ alkyl, —O—C₁₋₆ haloalkyl, ═O, and —CN;
R¹⁵ is independently selected at each occurrence from hydrogen, halogen, ═O, —CN, C₁₋₆ alkyl, C₁₋₆ haloalkyl, C₃₋₆ carbocycle, and 3- to 6-membered heterocycle; or two R 15 are taken together to form a C₃₋₆ carbocycle;
R¹⁶ is selected at each occurrence from hydrogen and C₁₋₆ alkyl;
R¹⁷ is independently selected at each occurrence from:
hydrogen;
C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═NR²¹, and —CN; and
C₃₋₁₀ carbocycle and 3- to 10-membered heterocycle, each of which is optionally substituted with one or more substituents independently selected from: halogen, C₁₋₆ alkyl, C₁₋₆ haloalkyl-OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂ (R²¹), —S(O)R²¹, —S(O)₂R²¹, —S(O)₂N(R²¹)₂, —NO₂, ═O, ═S, ═NR¹⁷, —CN, C₃₋₆ carbocycle, and 3- to 6-membered heterocycle; and
R¹⁸ and R²¹ are each independently selected at each occurrence from hydrogen, C₁₋₆ alkyl, C₁₋₆ haloalkyl, C₁₋₆ hydroxyalkyl, and C₁₋₆ aminoalkyl.

44. The compound or salt of claim 43, wherein A is:

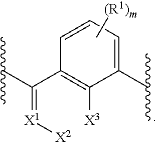

45. The compound or salt of claim 44, wherein A is selected from:

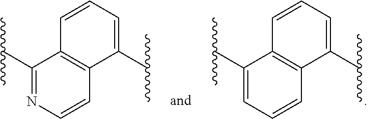 [SMILES: *c1cccc2c(*)nccc12 ; *c1cccc2c(*)cccc12]

46. The compound or salt of claim 45, wherein A is:

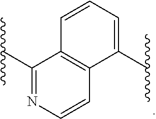 [SMILES: *c1cccc2c(*)nccc12]

47. The compound or salt of claim 45, wherein A is:

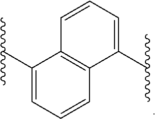 [SMILES: *c1cccc2c(*)cccc12]

48. The compound or salt of claim 43, wherein A is

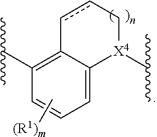

49. The compound or salt of claim 48, wherein A is: 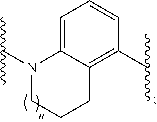
and n is 0 or 1.

50. The compound or salt of claim 43, wherein Y is a bond.

51. The compound or salt of claim 43, wherein R^{4a} and R^{4b} are each independently selected from hydrogen, halogen, C₁₋₃ alkyl, and C₁₋₃ haloalkyl.

52. The compound or salt of claim 51, wherein R^{4a} and R^{4b} are each hydrogen or halogen.

53. The compound or salt of claim 43, wherein R⁵ is hydrogen.

54. The compound or salt of claim 43, wherein R³ is hydrogen, methyl, or ethyl.

55. The compound or salt of claim 54, wherein R³ is hydrogen.

56. The compound or salt of claim 43, wherein Z is selected from: a bond, —C(O)—, —C(R¹⁵)₂—, —C(O)C(R¹⁵)₂—, —C(O)[C(R¹⁵)₂]ₚS—, —C(═N—CN)—, and —S(O)₂—.

57. The compound or salt of claim 56, wherein Z is —C(O)—.

58. The compound or salt of claim 43, wherein B is selected from: phenyl, naphthyl, C₃₋₁₀ cycloalkyl, 5- to 6-membered monocyclic heterocycle, and 7- to 10-membered bicyclic heterocycle, any of which is optionally substituted.

59. The compound or salt of claim 58, wherein B is selected from: phenyl, naphthyl, cyclopropyl, cyclohexyl, bicyclo[2.2.2]octane, adamantane, thiazole, pyrrole, pyrazole, pyridine, pyrimidine, pyrazine, indole, indazole, benzimidazole, indoline, azaindole, azaindoline, 1,3-dihydrobenzo[c]isothiazole, tetrahydropyran, azetidine, pyrrolidine, piperidine, quinuclidine, azepane, and 8-azabicyclo[3.2.1]octane, any of which is optionally substituted.

60. The compound or salt of claim 59, wherein B is selected from: phenyl, cyclohexyl, pyrazole, pyridine, indole, indazole, benzimidazole, indoline, 1,3-dihydrobenzo[c]isothiazole, pyrrolidine, piperidine, pyrrolidine, and piperidine, any of which is optionally substituted.

61. The compound or salt of claim 43, wherein B is substituted with 1, 2, or 3 substituents.

62. The compound or salt of claim 61, wherein B is substituted with 2 or 3 substituents.

63. The compound or salt of claim 43, wherein any substituents on B are independently selected at each occurrence from:
halogen, —OR¹⁷—SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂ (R¹⁷), —S(O)R¹⁷, —S(O)₂R¹⁷, —S(O)₂N(R¹⁷)₂, and —CN;
C₁₋₆ alkyl optionally substituted with one or more substituents independently selected from:
halogen, —OR¹⁷, —SR¹⁷, —N(R¹⁷)₂, —C(O)R¹⁷, —N(R¹⁷)C(O)R¹⁷, —N(R¹⁷)C(O)OR¹⁷, —N(R¹⁷)S(O)₂ (R¹⁷), —S(O)₂N(R¹⁷)₂, ═O, —CN; and
C₃₋₁₀ carbocycle and 3- to 10-membered heterocycle each of which is optionally substituted with one or more substituents independently selected from: halogen, C₁₋₆ alkyl, C₁₋₆ haloalkyl, —OR¹⁷, —SR¹⁷, —N(R¹⁷)₂, —C(O)R¹⁷, —C(O)OR¹⁷, —C(O)N(R¹⁷)₂, —N(R¹⁷)S(O)₂ (R¹⁷), —S(O)₂R¹⁷, ═O, ═S, and —CN; and
C₃₋₁₀ carbocycle and 3- to 10-membered heterocycle each of which is optionally substituted with one or more substituents independently selected from:
halogen, —OR¹⁷, —SR¹⁷, —N(R¹⁷)₂, —C(O)R¹⁷, —C(O)OR¹⁷, —C(O)N(R¹⁷)₂, —(R¹⁷)S(O)₂ (R¹⁷), —S(O)R¹⁷, —S(O)₂R¹⁷, ═O, ═S, —CN; and
C₁₋₆ alkyl optionally substituted with one or more substituents independently selected from: halogen, —OR¹⁷, —SR¹⁷, —N(R¹⁷)₂, ═O, ═S, and —CN.

64. The compound or salt of claim 63, wherein any substituents on B are independently selected at each occurrence from:
halogen, —OR¹⁷, —N(R¹⁷)₂, —C(O)R¹⁷, —C(O)N(R¹⁷)₂, —N(R¹⁷)S(O)₂ (R¹⁷), —S(O)₂R¹⁷, and —CN;
C₁₋₆ alkyl optionally substituted with one or more substituents independently selected from: halogen and 3- to 10-membered heterocycle optionally substituted with one or more substituents independently selected from halogen, C₁₋₆ alkyl, and C₁₋₆ haloalkyl; and
C₃₋₁₀ carbocycle and 3- to 10-membered heterocycle, each of which is optionally substituted with one or more substituents independently selected from: halogen, C₁₋₆ alkyl, and C₁₋₆ haloalkyl.

65. The compound or salt of claim 64, wherein any substituents on B are independently selected at each occurrence from:
halogen, —N(R¹⁷)₂, —N(R¹⁷)S(O)₂ (R¹⁷), and —CN;
C₁₋₆ alkyl optionally substituted with one or more substituents independently selected from halogen and 3- to 10-membered heterocycle optionally substituted with one or more substituents independently selected from halogen and C₁₋₆ alkyl; and
3- to 10-membered heterocycle optionally substituted with one or more substituents independently selected from C₁₋₆ alkyl and C₁₋₆ haloalkyl.

66. The compound or salt of claim 43, wherein R¹⁷ is independently selected at each occurrence from:
hydrogen;
C₁₋₆ alkyl optionally substituted with one or more substituents independently selected from, halogen, —OR²¹, —SR²¹, —N(R²¹)₂, and —CN; and
C₃₋₁₀ carbocycle and 3- to 10-membered heterocycle, each of which is optionally substituted with one or more substituents independently selected from: halogen, C₁₋₆ alkyl, C₁₋₆ haloalkyl, —OC(O)N(R²¹)₂, and C₃₋₆ carbocycle.

67. The compound or salt of claim 43, wherein R²¹ is independently selected at each occurrence from hydrogen and C₁₋₆ alkyl.

68. The compound or salt of claim 63, wherein any substituents on B are independently selected at each occurrence from: halogen, C₁₋₆ alkyl, C₁₋₆ haloalkyl, O—C₁₋₆ alkyl, —O—C₁₋₆ haloalkyl, ═O, —CN, acetyl, difluoroacetyl,

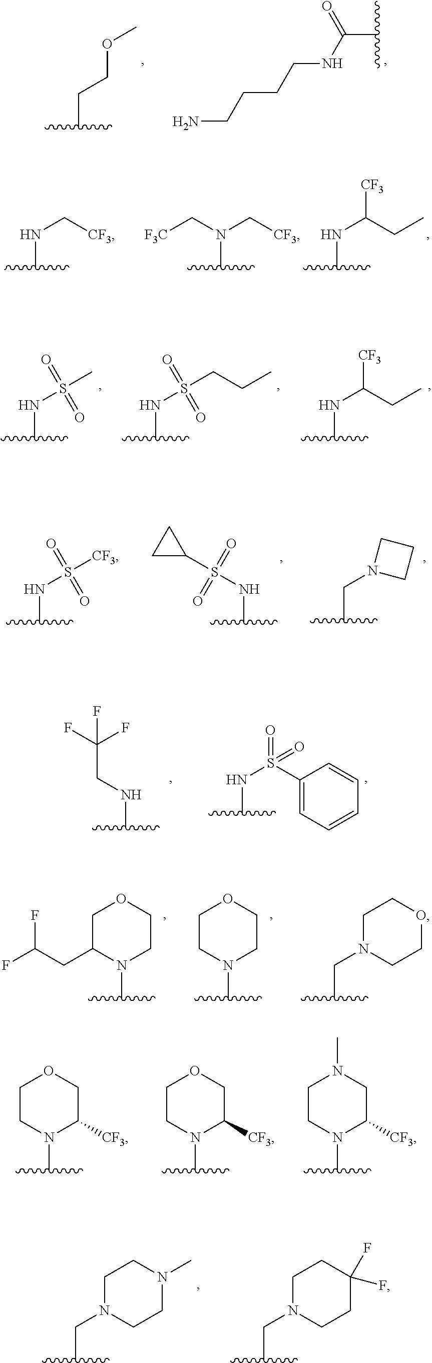

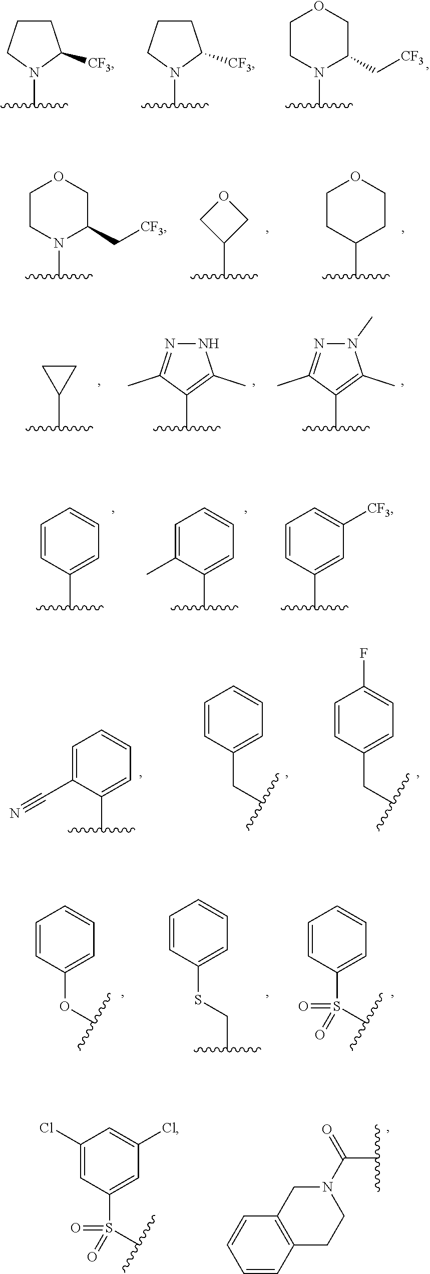

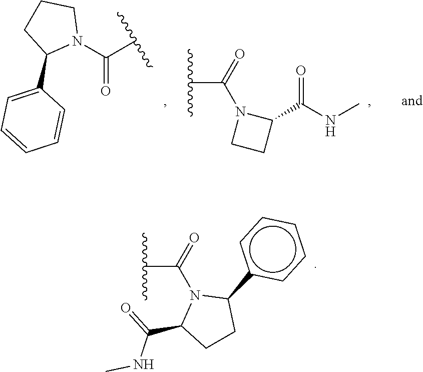

69. The compound or salt of claim 43, wherein B is

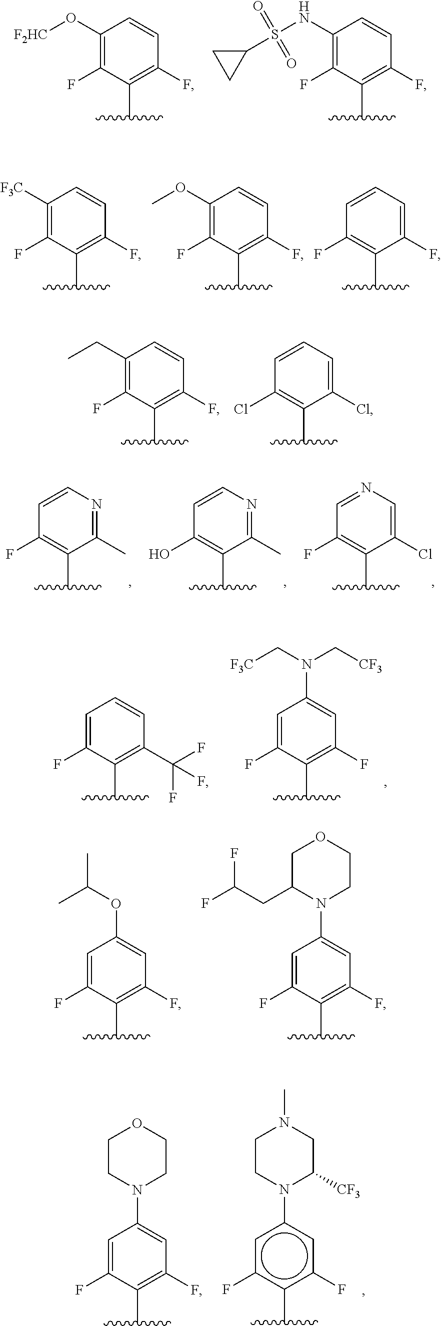

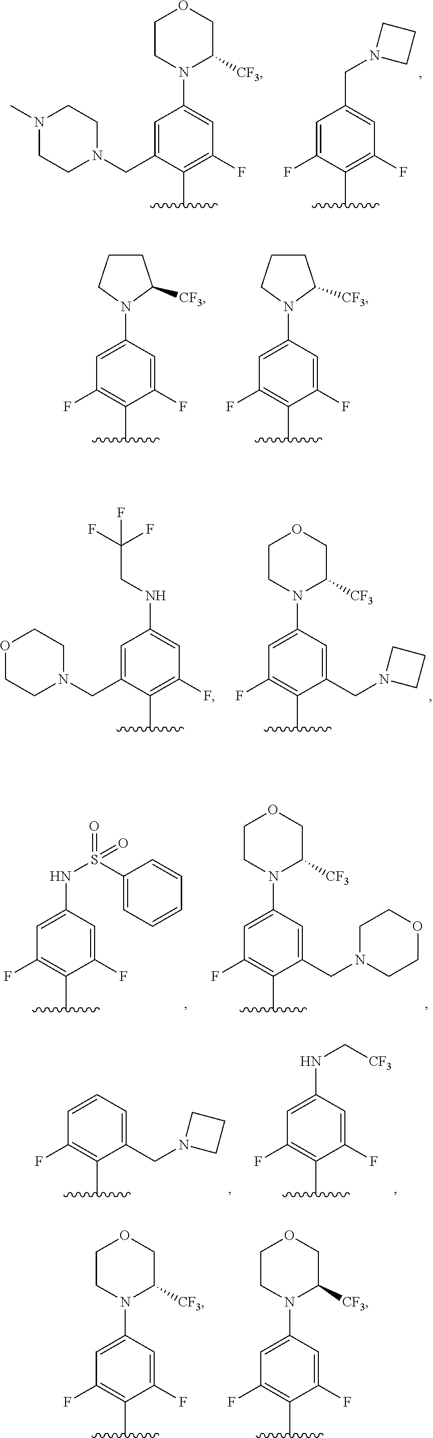

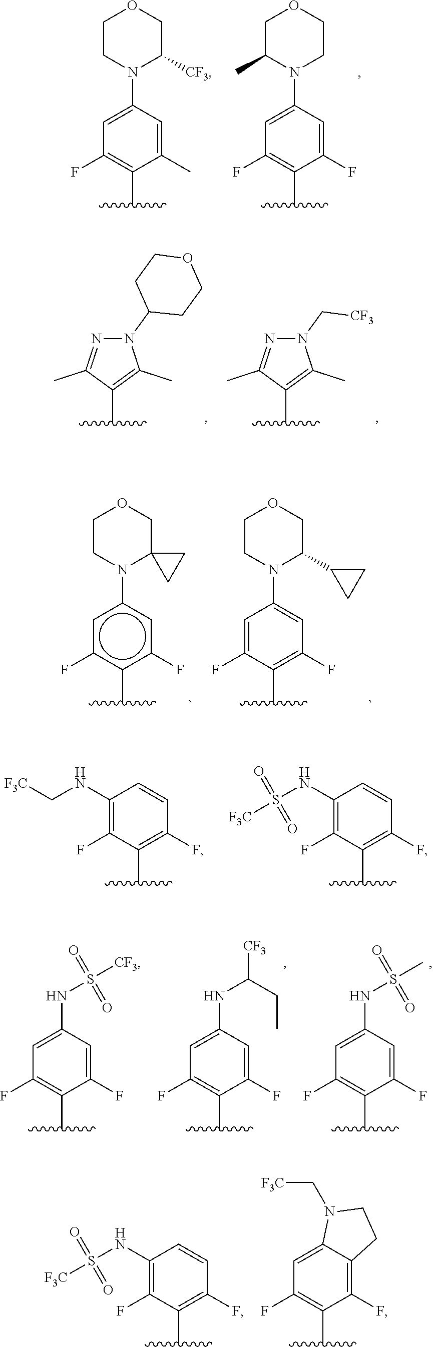

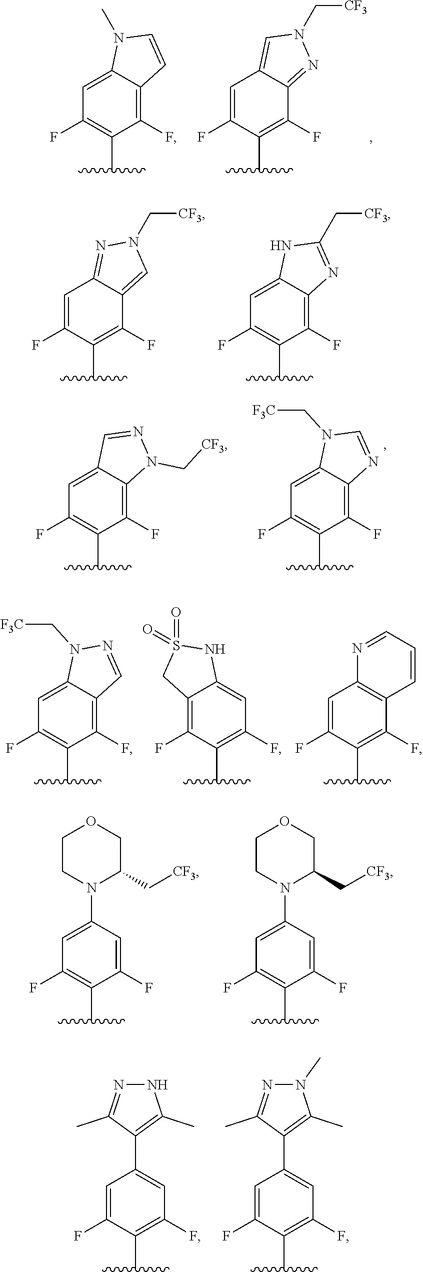

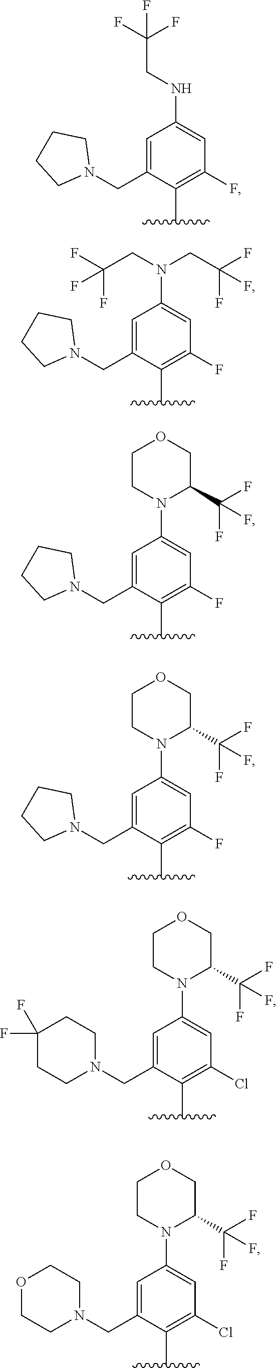

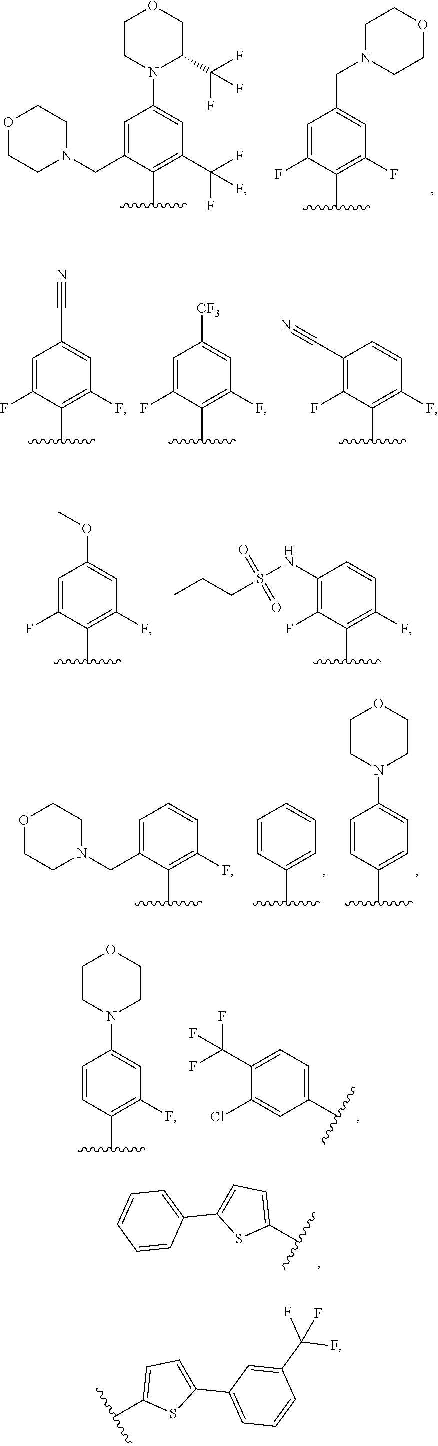

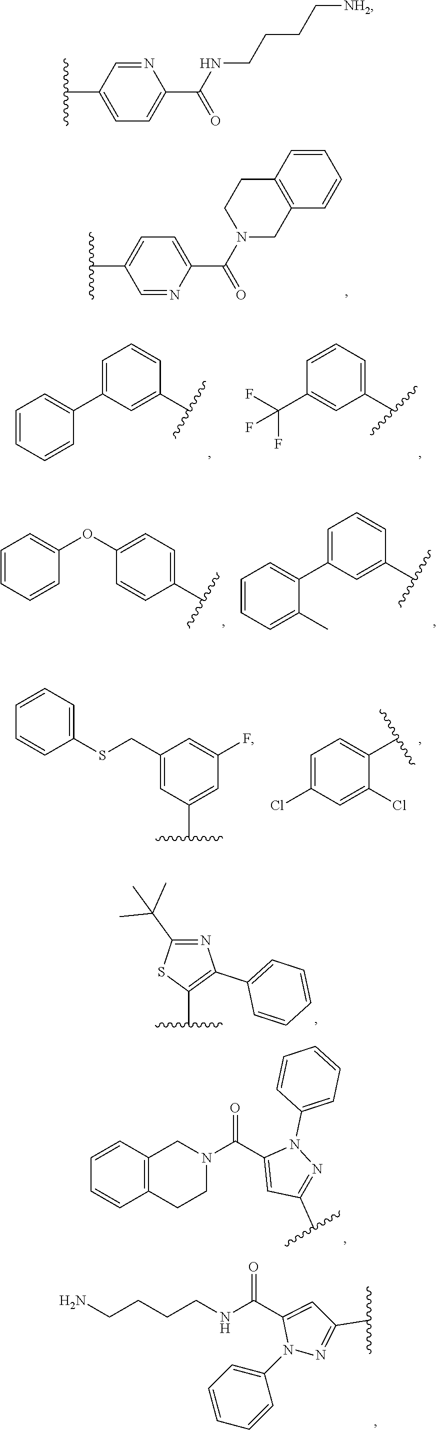

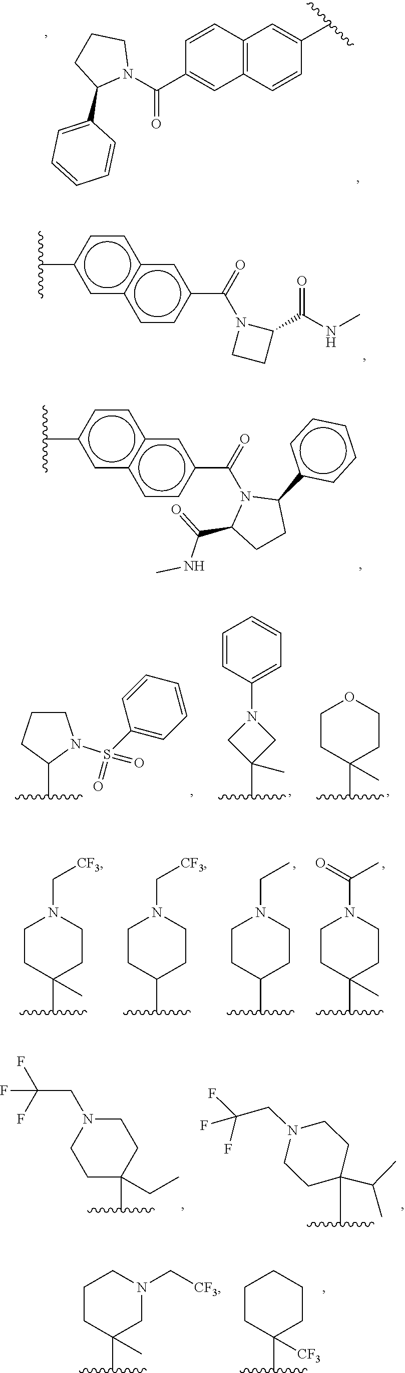

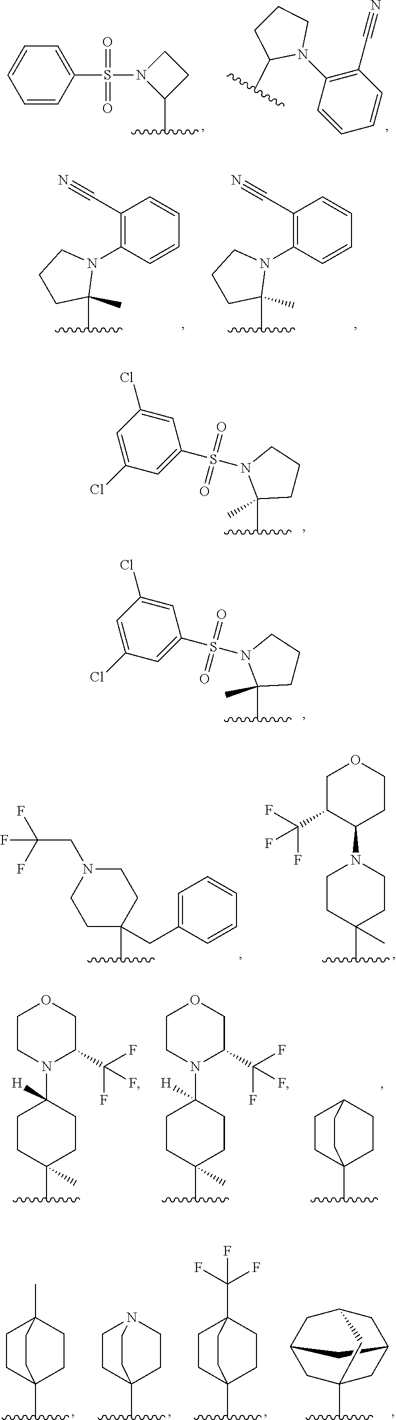

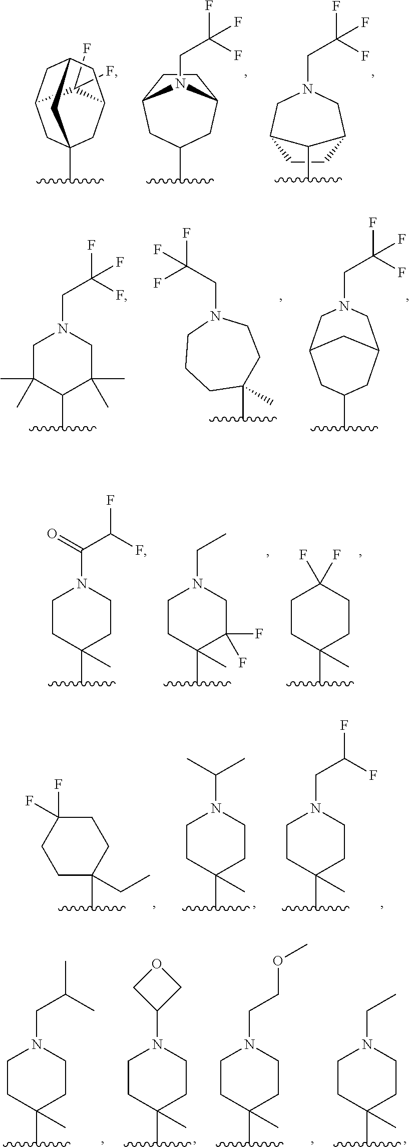

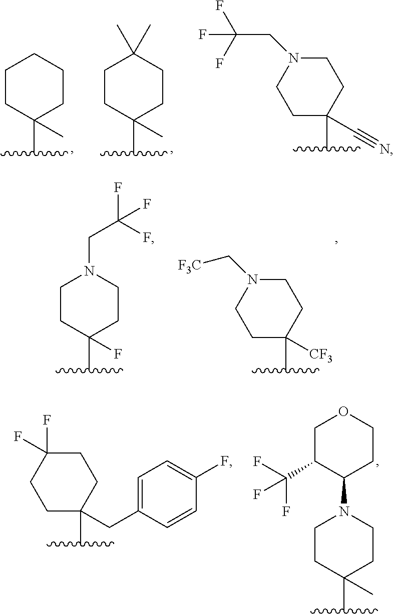

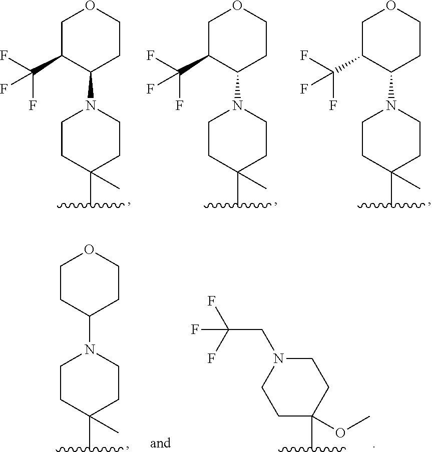

70. The compound or salt of claim 43, wherein B is

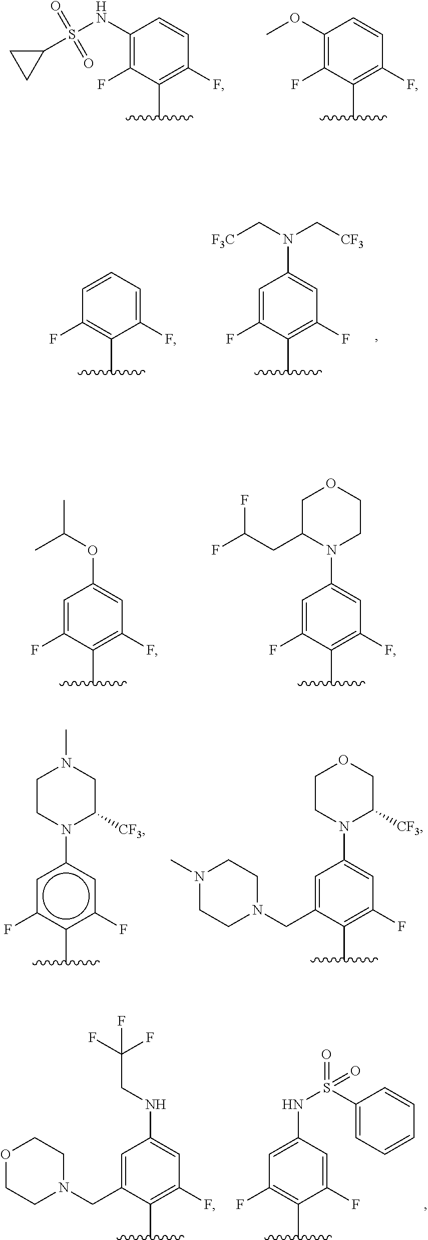

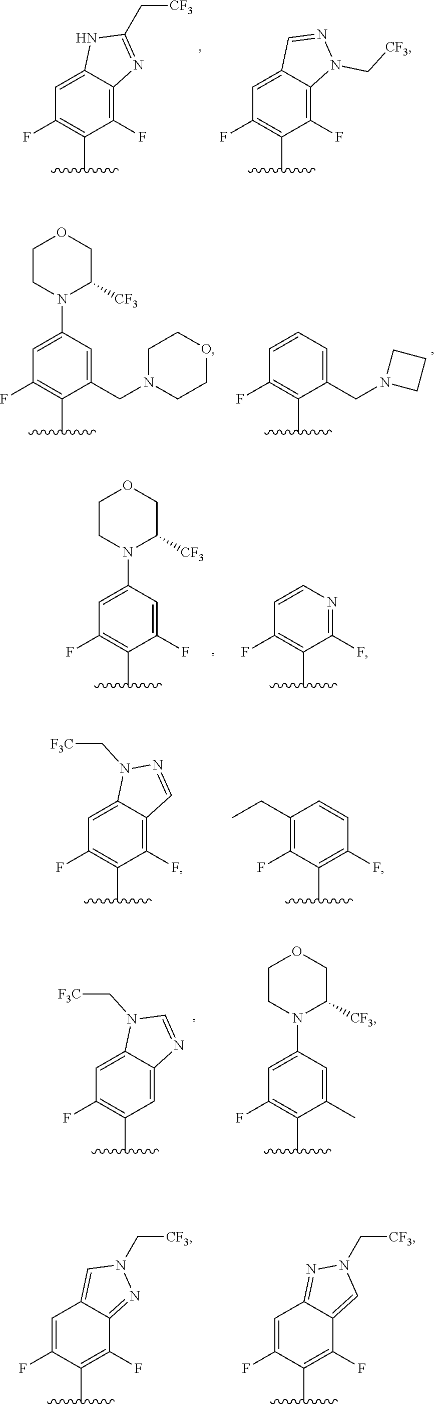

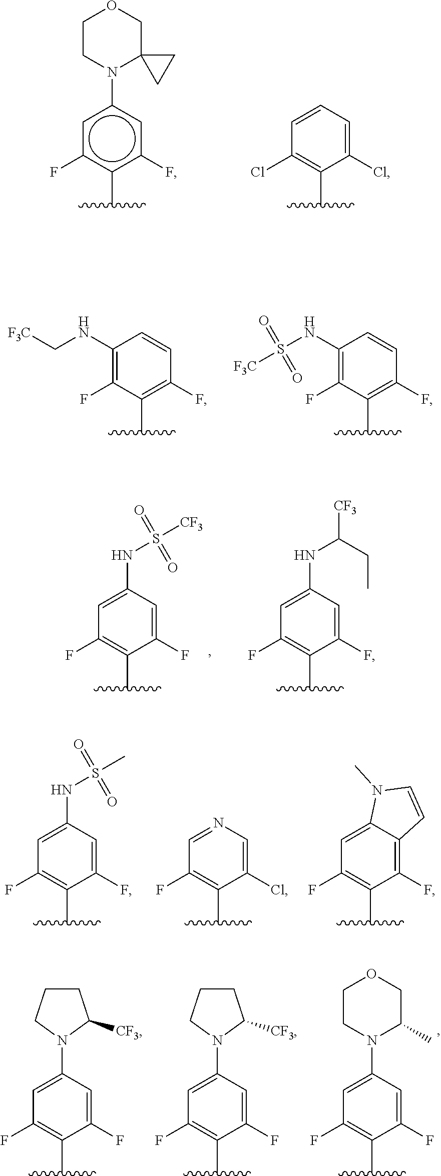

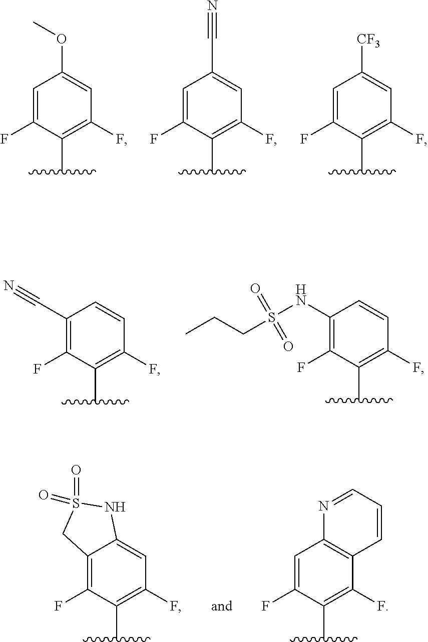

71. The compound or salt of claim 43, wherein B is selected from:

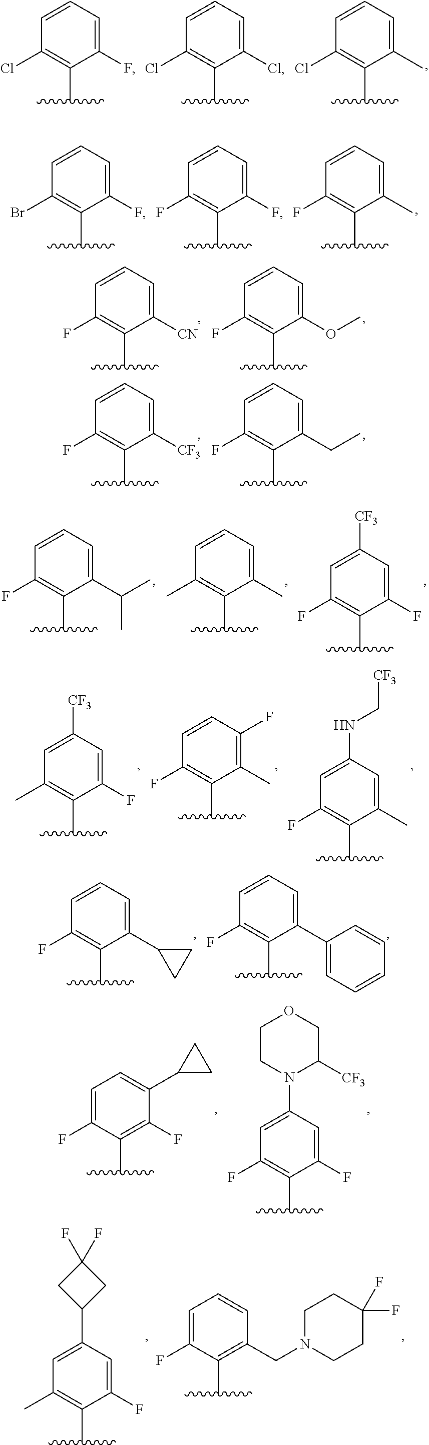

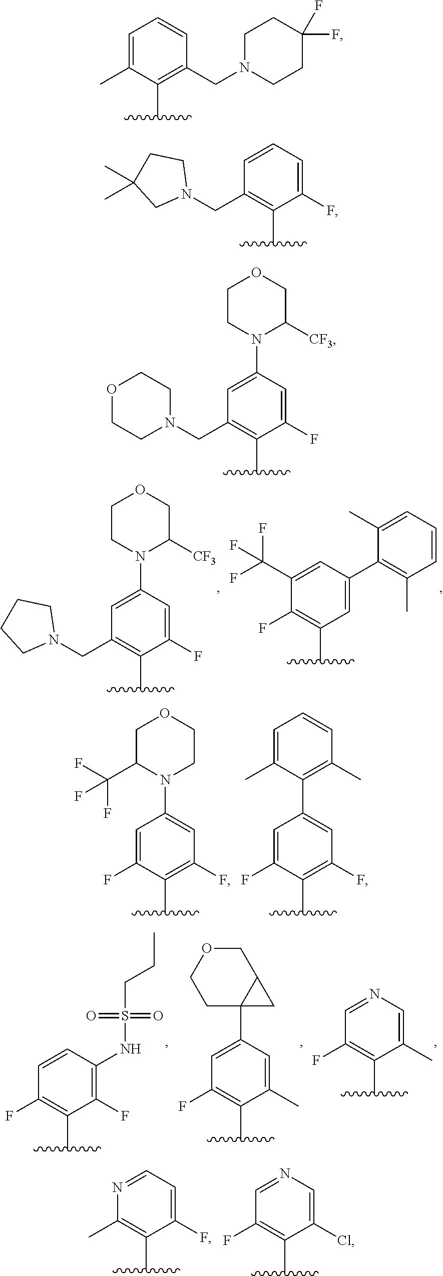

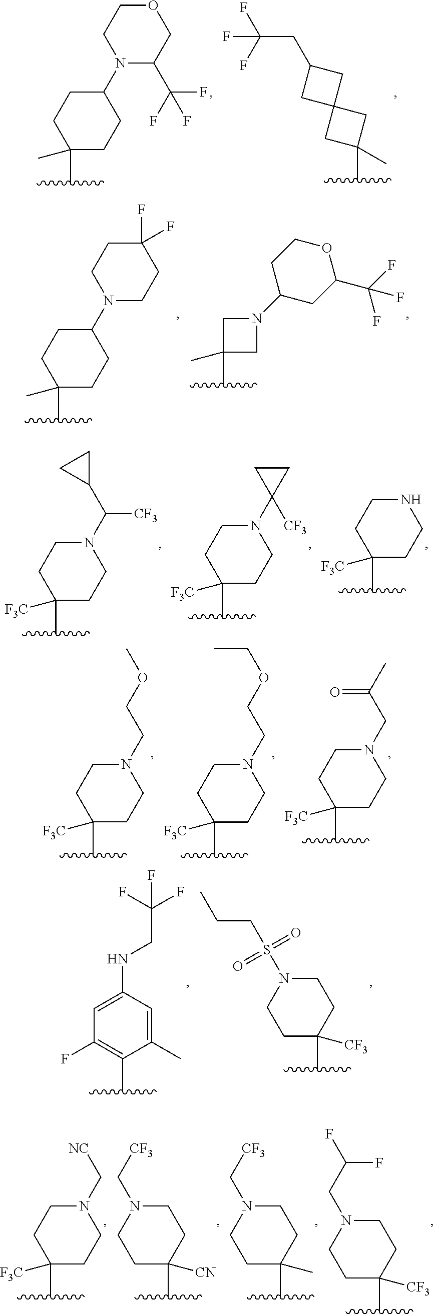

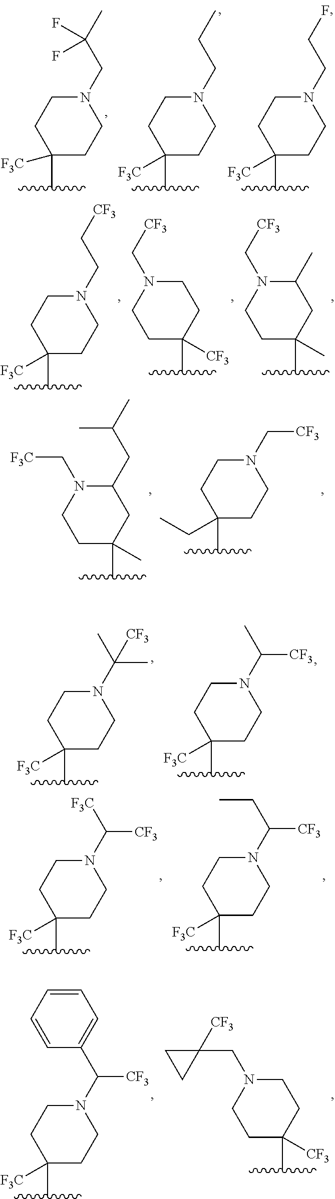

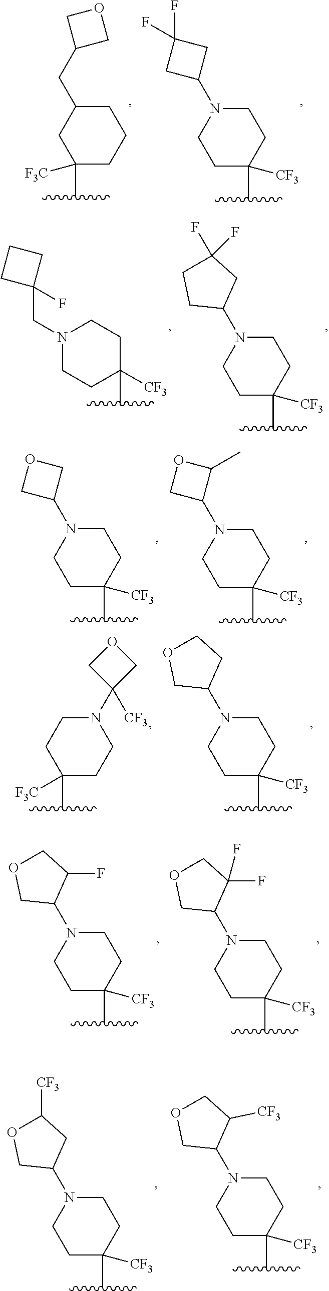

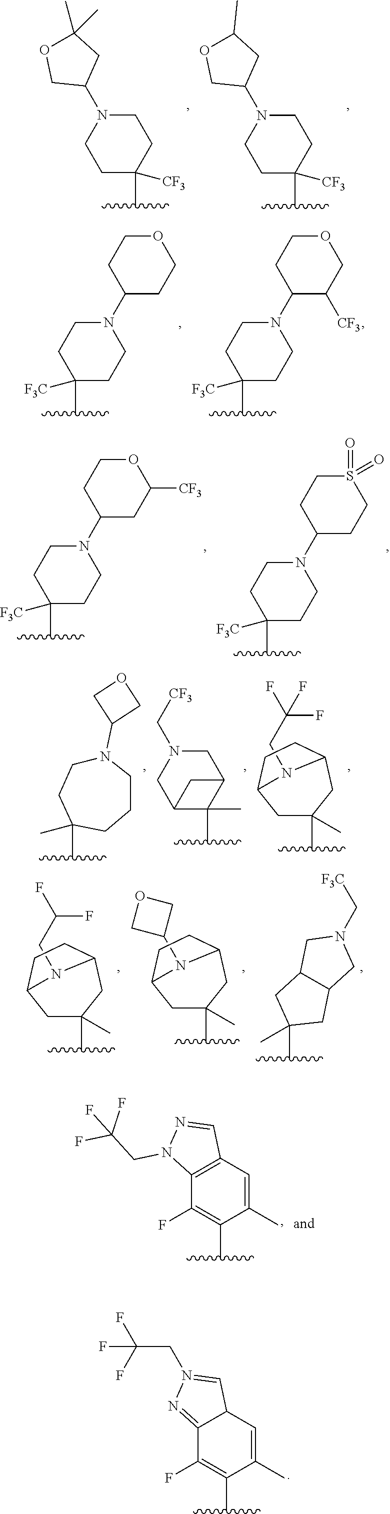

72. The compound or salt of claim 43, wherein C is selected from phenyl, 5-6-membered monocyclic heterocycle, and 7-10-membered bicyclic heterocycle, each of which is optionally substituted.

73. The compound or salt of claim 71, wherein C is selected from phenyl, pyridine, pyridin-2-one, pyrimidone, pyrimidinedione, pyrazine, pyridazin-3-one, isoquinoline, naphthyridine, phthalazine, naphthyridin-2-one, 5,6,7,8-tetrahydronaphthyridin-2-one, triazolo[1,5-a]pyrazine, imidazo[1,2-a]pyrazine, pyrazolo[1,5-a]pyrazine, pyrido[4,3-d]pyrimidine-2,4-dione, pyrido[3,4-d]pyrimidine-2,4-dione, quinazoline-2,4-dione, quinolin-2-one, 3,4-dihydroquinolin-2-one, isoindolin-1-one, and 1,3-dihydro-benzo[d]imidazol-2-one, each of which is optionally substituted.

74. The compound or salt of claim 1, wherein the one or more optional substituents on C are independently selected at each occurrence from:
halogen, —OR¹⁸, —SR¹⁸, —N(R¹⁸)₂, —C(O)R¹⁸, —C(O)OR¹⁸, —C(O)N(R¹⁸)₂, —S(O)R¹⁸, —S(O)₂R¹⁸, —NO₂, ═O, ═S, and —CN;
C₁₋₆ alkyl and C₂₋₆ alkenyl, each of which is optionally substituted with one or more substituents independently selected from: halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂ (R¹⁸), —S(O)₂N(R¹⁸)₂, —NO₂, ═O, ═S, —CN, and 3- to 6-membered heterocycle optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, and C₁₋₆ haloalkyl.

75. The compound or salt of claim 74, wherein the optional substituents on C are independently selected at each occurrence from:
halogen, —OR¹⁸, —N(R¹⁸)₂, ═O, and —CN;
C₁₋₆ alkyl and C₂₋₆ alkenyl, each of which is optionally substituted with one or more substituents independently selected from: halogen, —OR¹⁸, —N(R¹⁸)₂, ═O, —CN, and 3- to 6-membered heterocycle optionally substituted with one or more substituents independently selected from: halogen, C₁₋₄ alkyl, and C₁₋₄ haloalkyl; and
C₃₋₆ carbocycle and 3- to 6-membered heterocycle, each of which is optionally substituted with one or more substituents independently selected from halogen, C₁₋₆ alkyl, and C₁₋₆ haloalkyl.

76. The compound or salt of claim 43, wherein R¹⁸ is independently selected at each occurrence from hydrogen and C₁₋₆ alkyl.

77. The compound or salt of claim 43, wherein the optional substituents on C are independently selected at each occurrence from: halogen, ═O, —N(CH₃)₂, C₁₋₆ alkyl, C₂₋₆ alkenyl, C₁₋₆ haloalkyl, —O—C₁₋₆ alkyl, —O—C₁₋₆ haloalkyl,

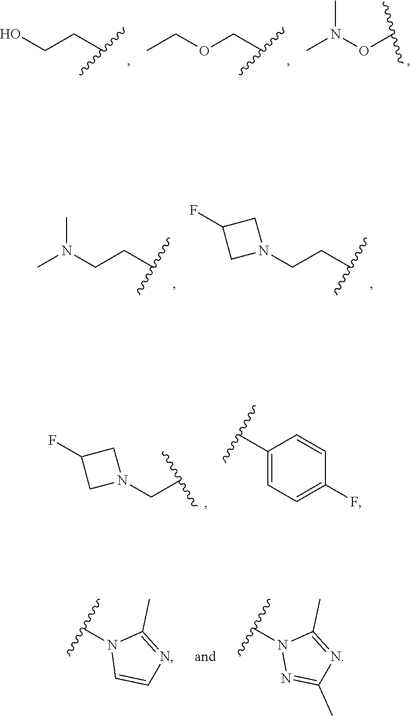

78. The compound or salt of claim 43, wherein C is

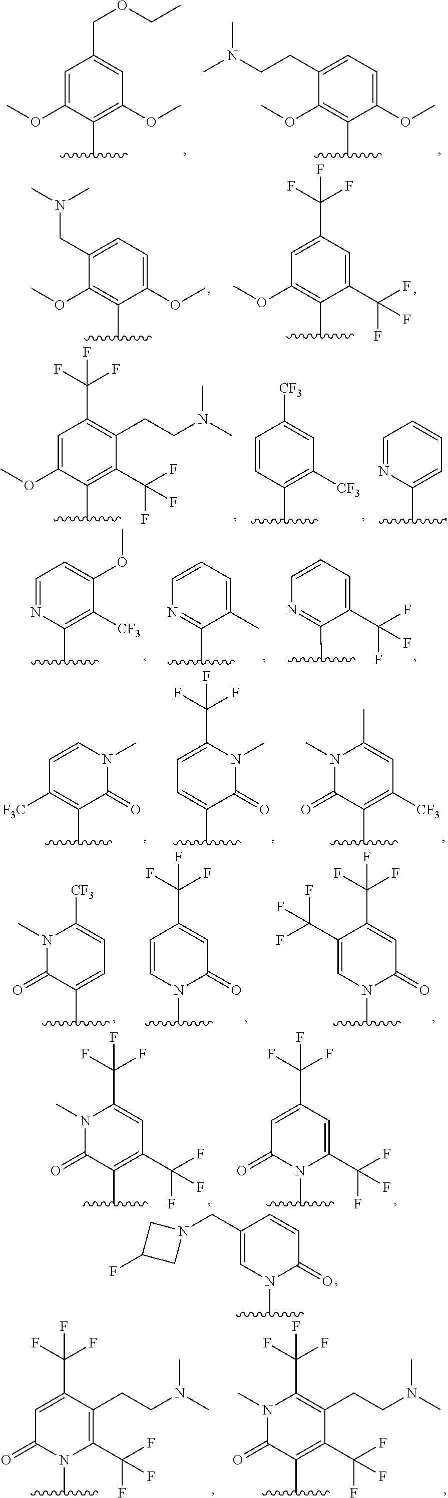

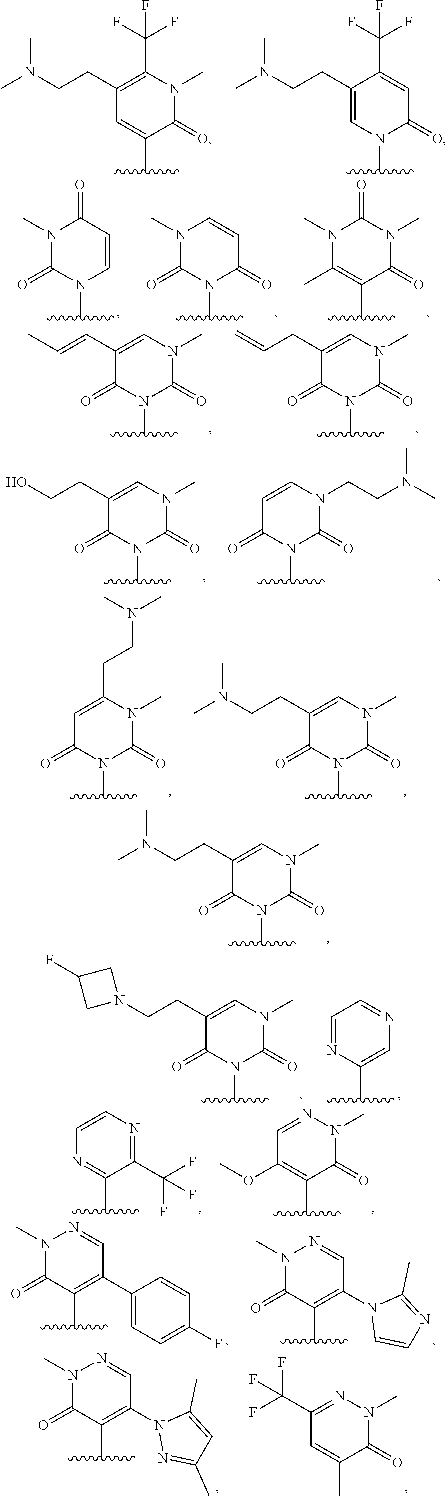

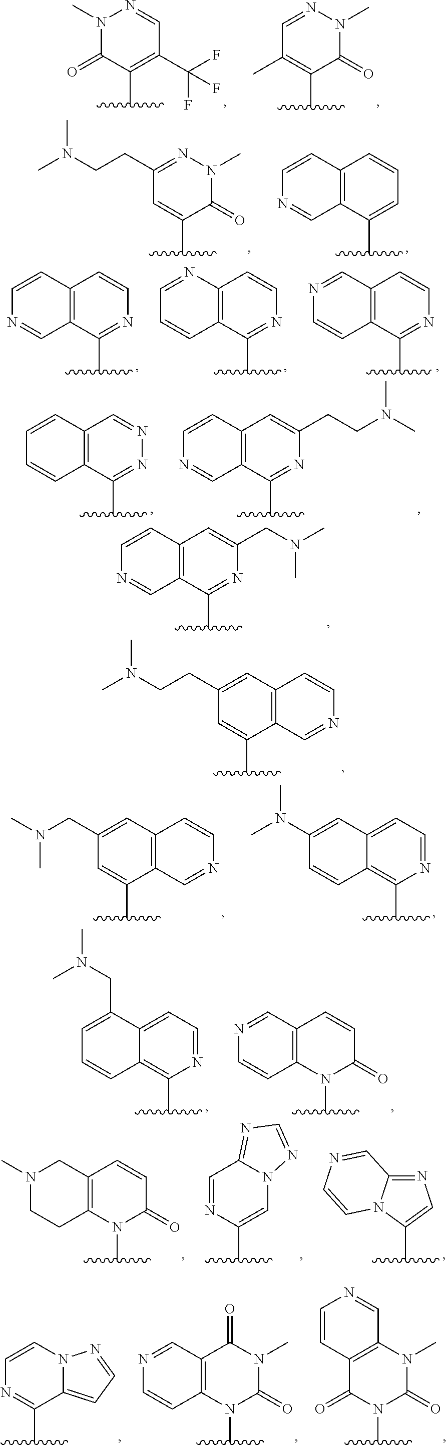

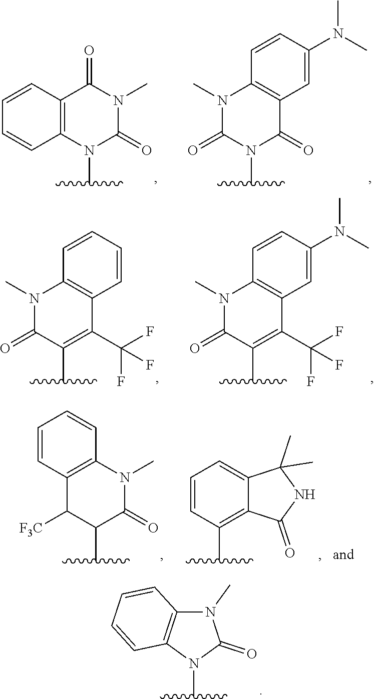

79. The compound or salt of claim 43, wherein C is selected from:

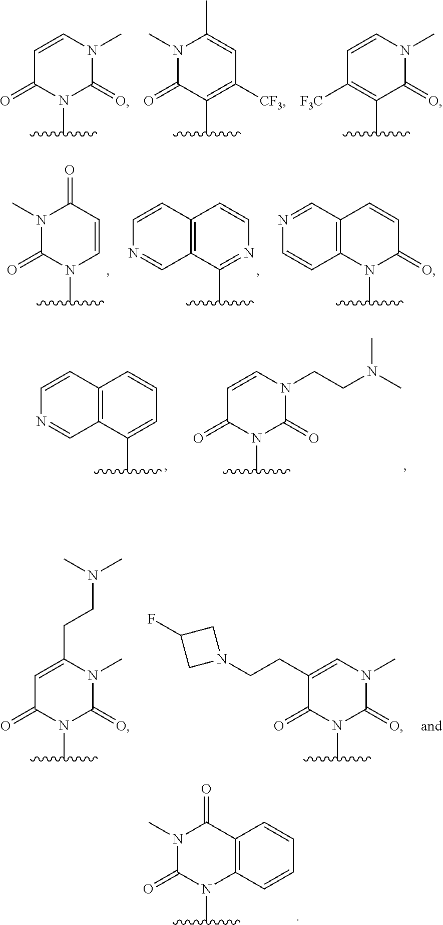

80. The compound or salt of claim 43, wherein C is

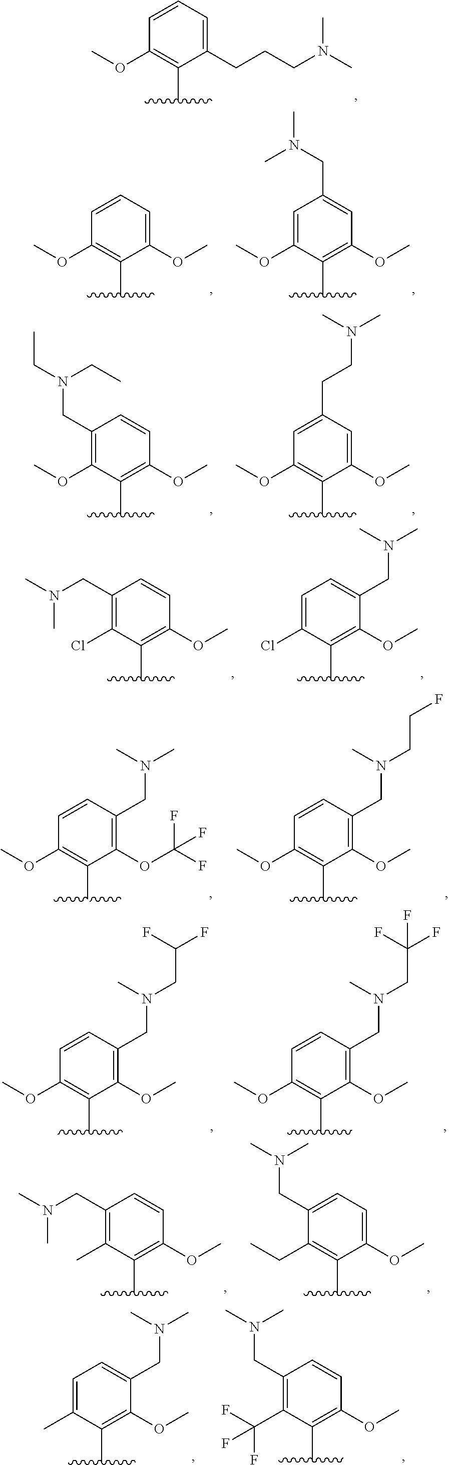

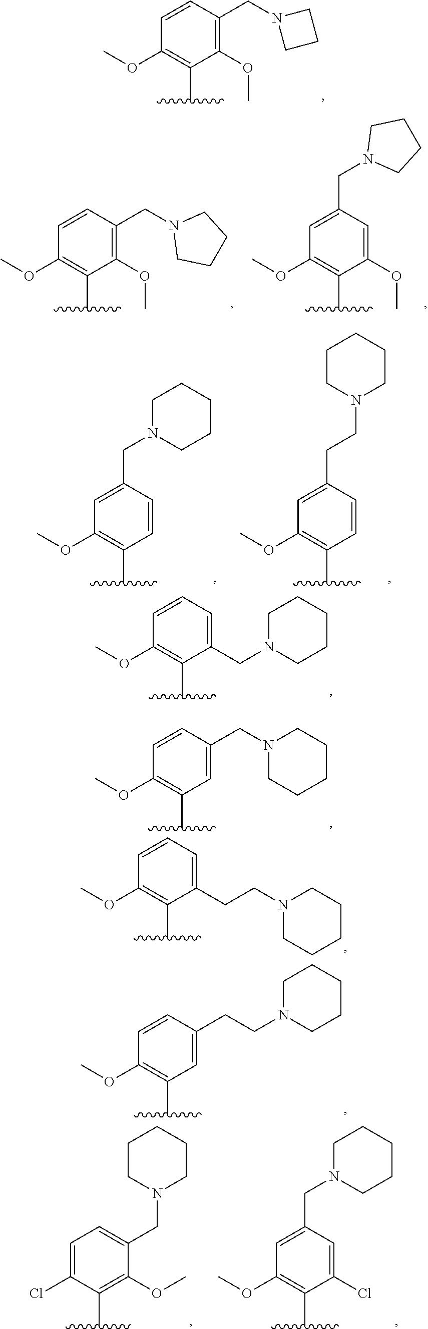

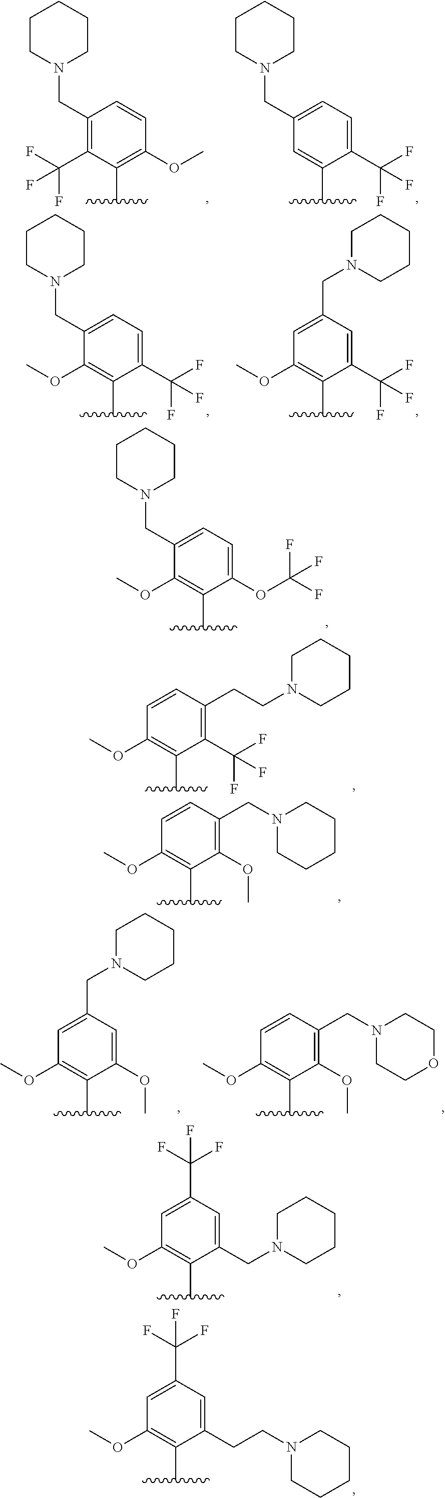

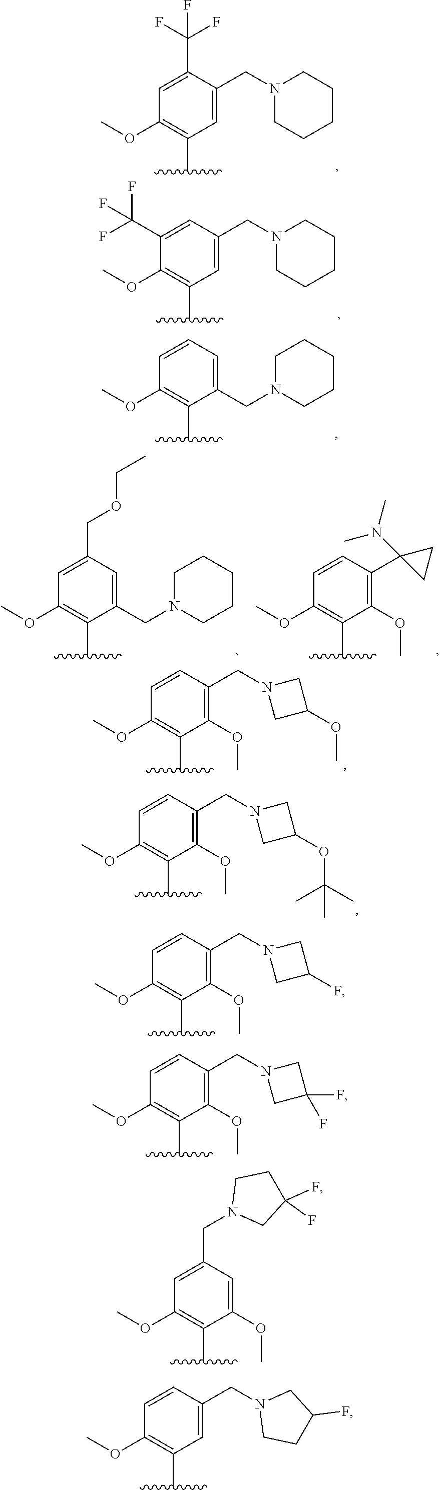

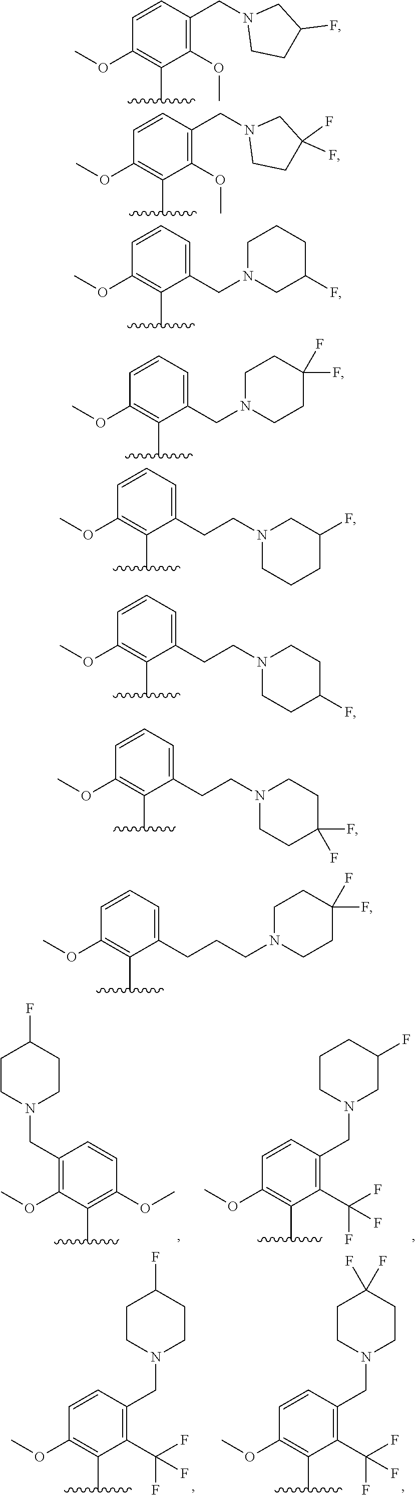

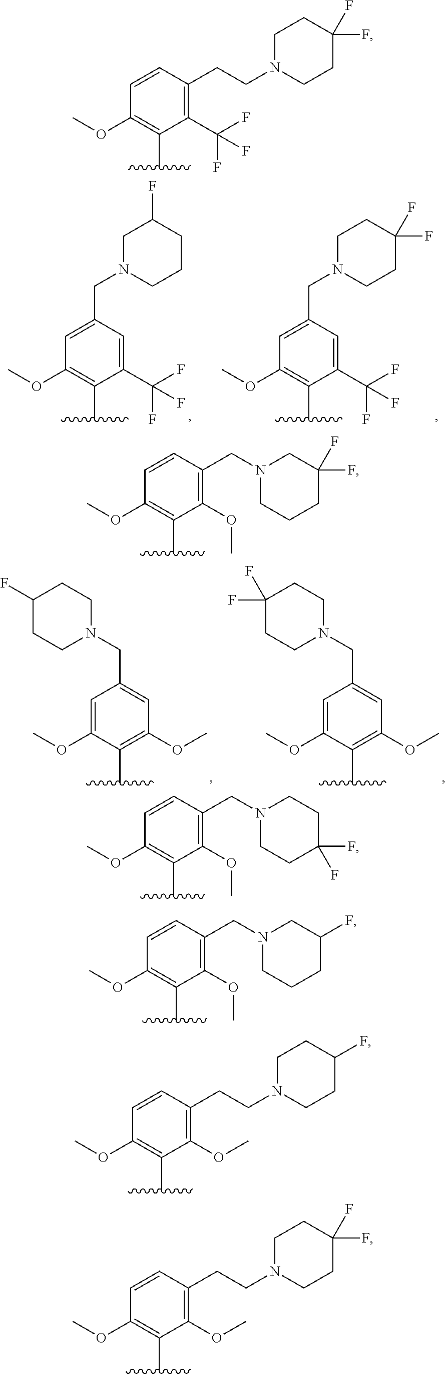

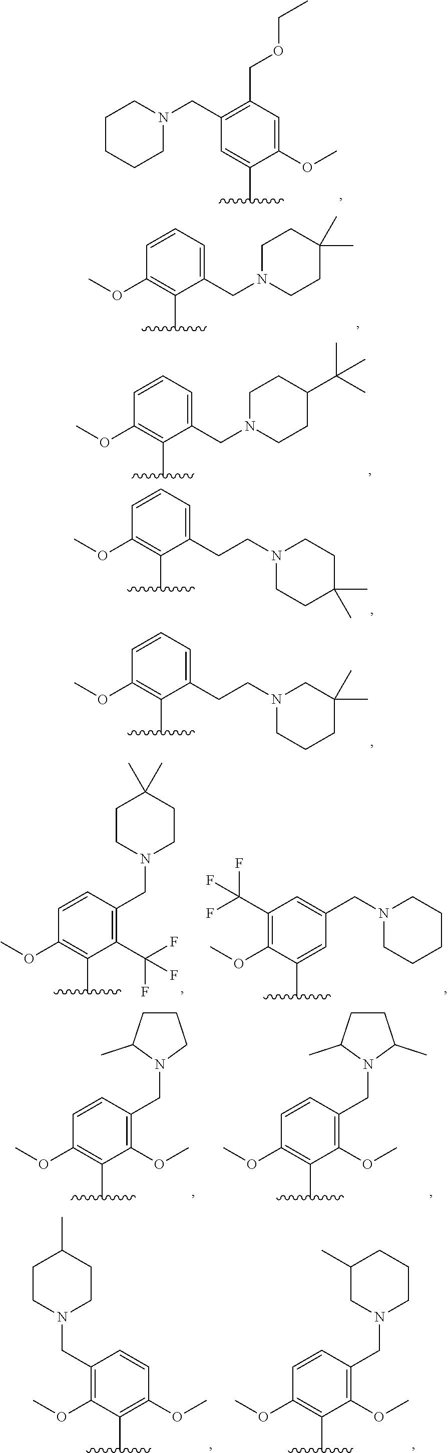

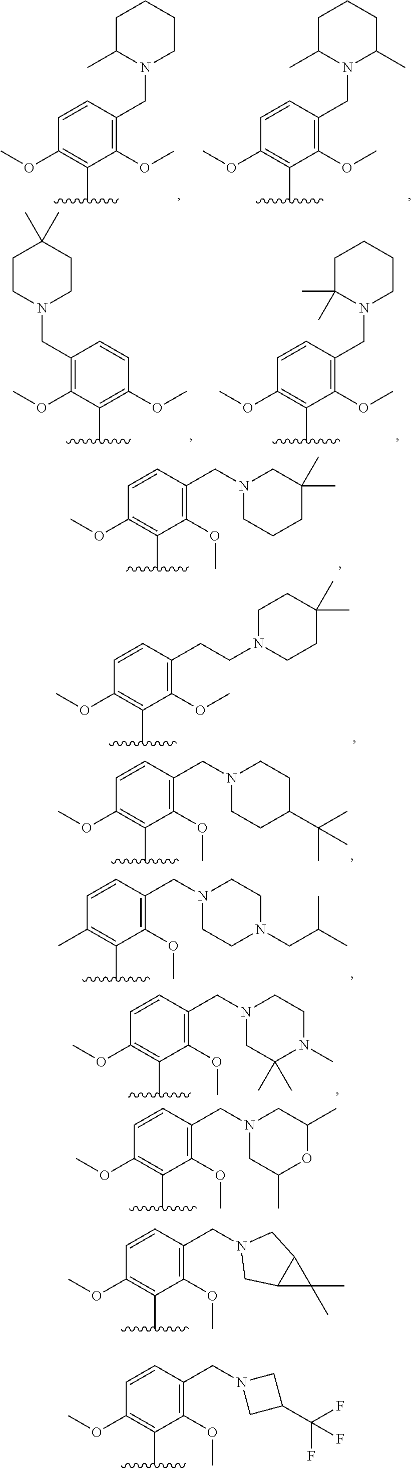

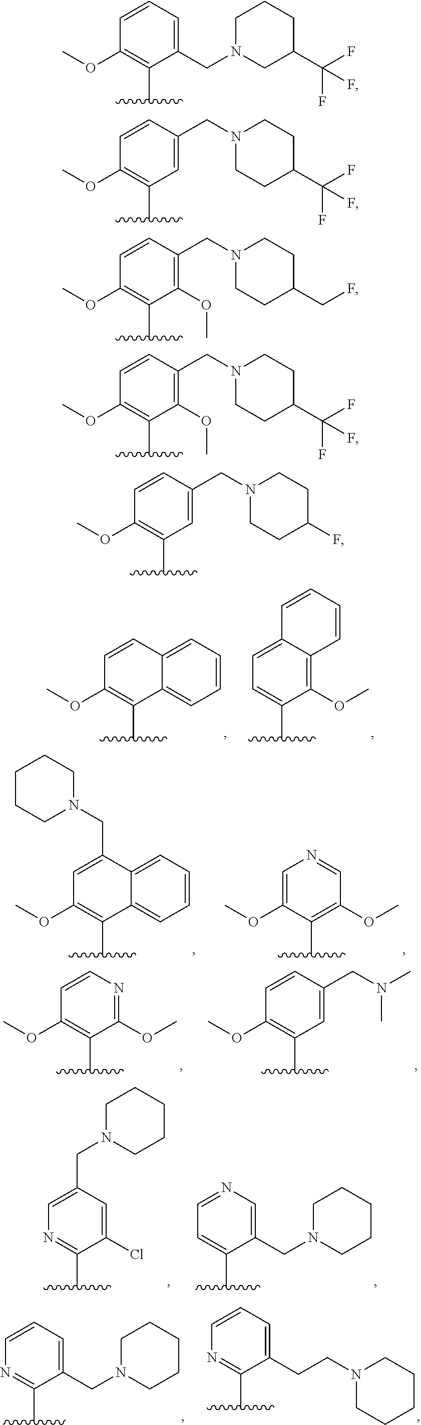

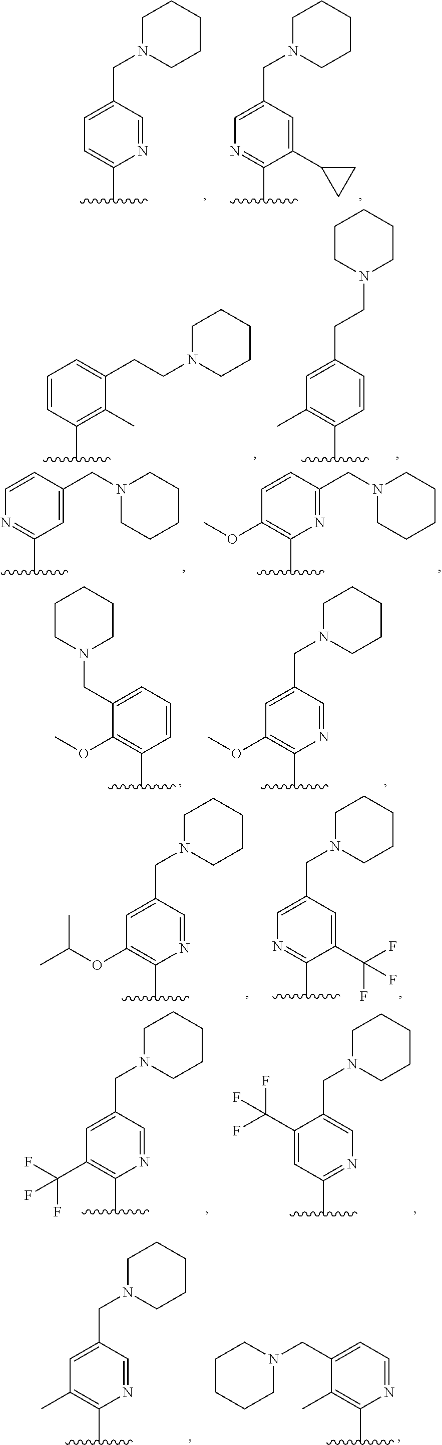

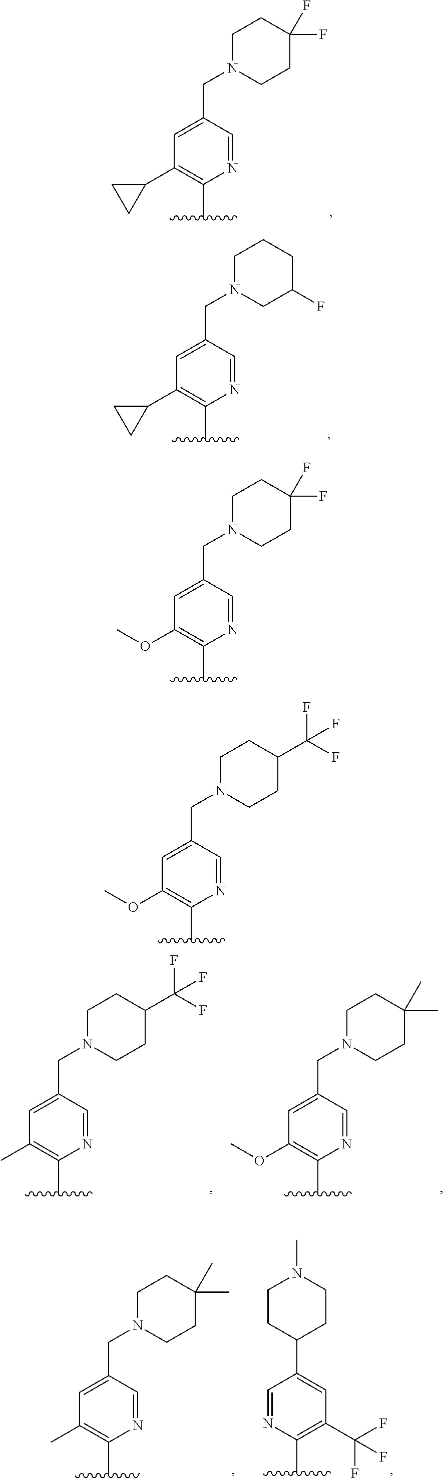

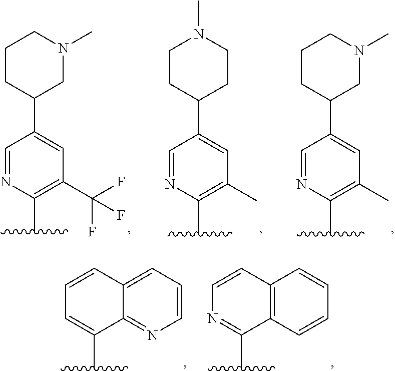

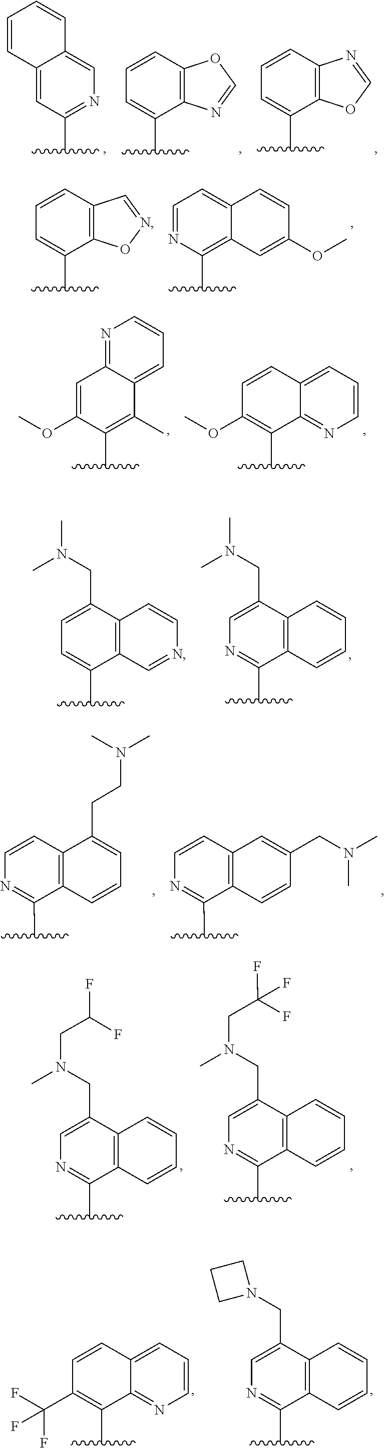

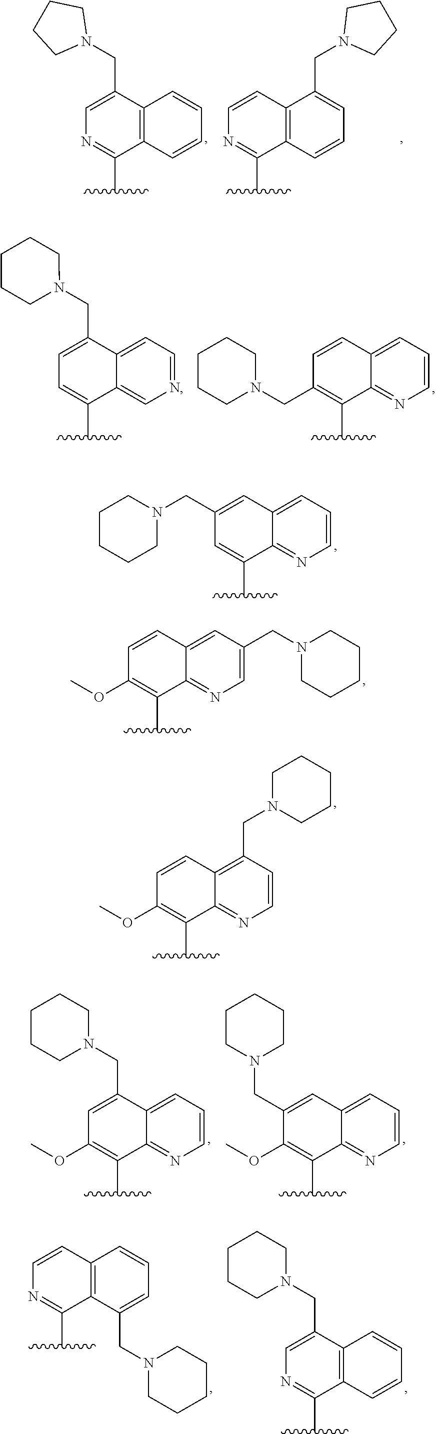

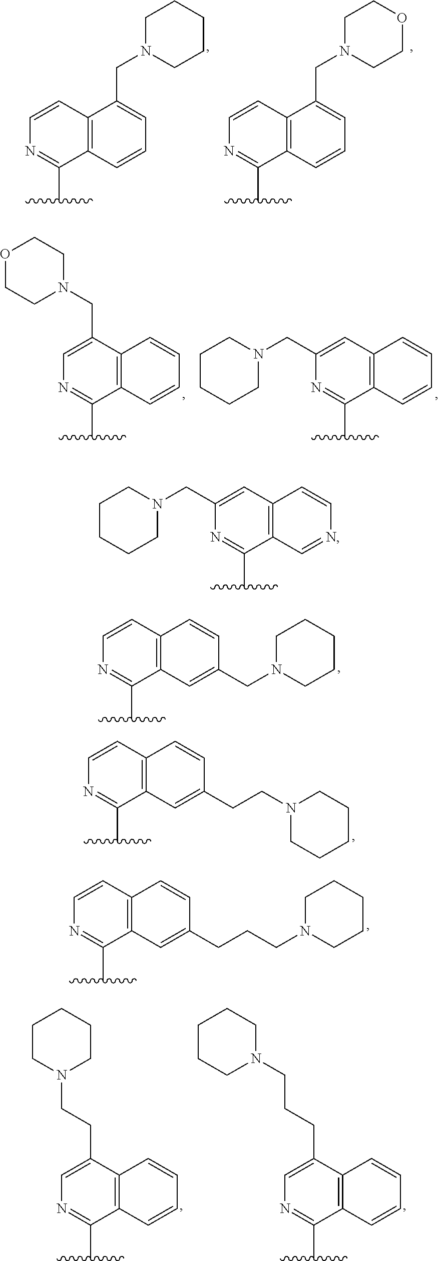

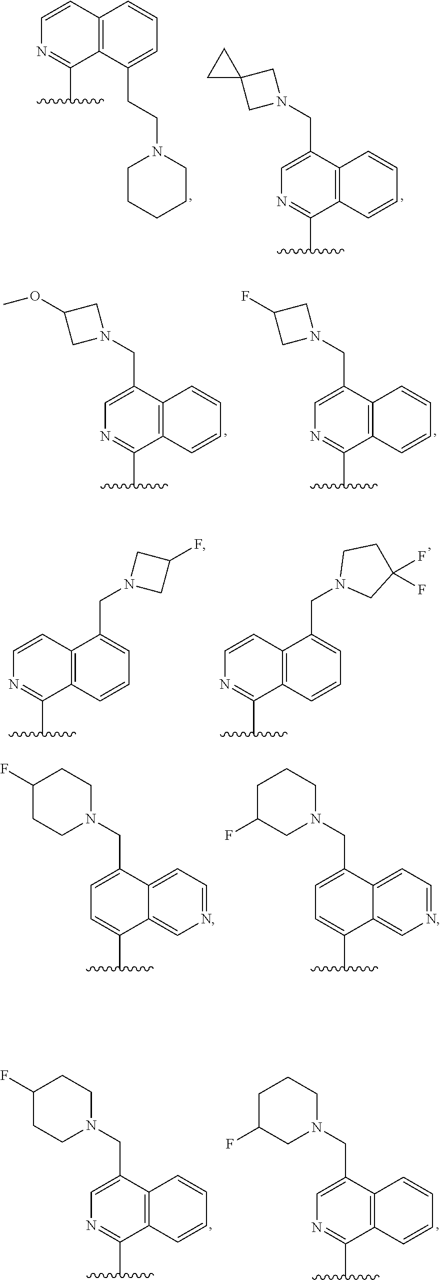

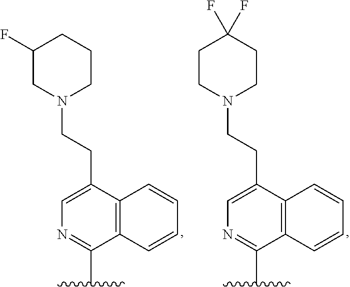 [SMILES: *c1ncc(CCN2CCCC(F)C2)c2ccccc12 ; *c1ncc(CCN2CCC(F)(F)CC2)c2ccccc12]

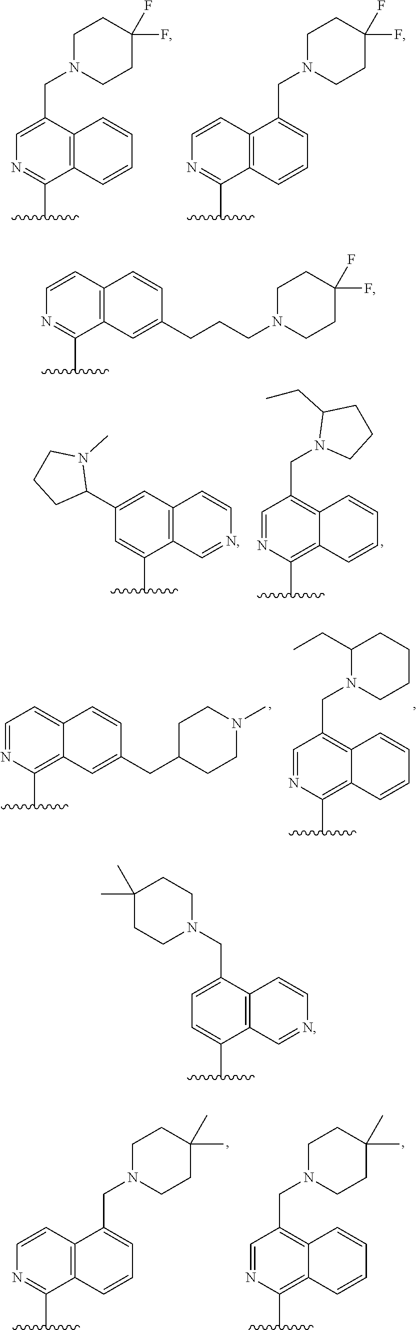

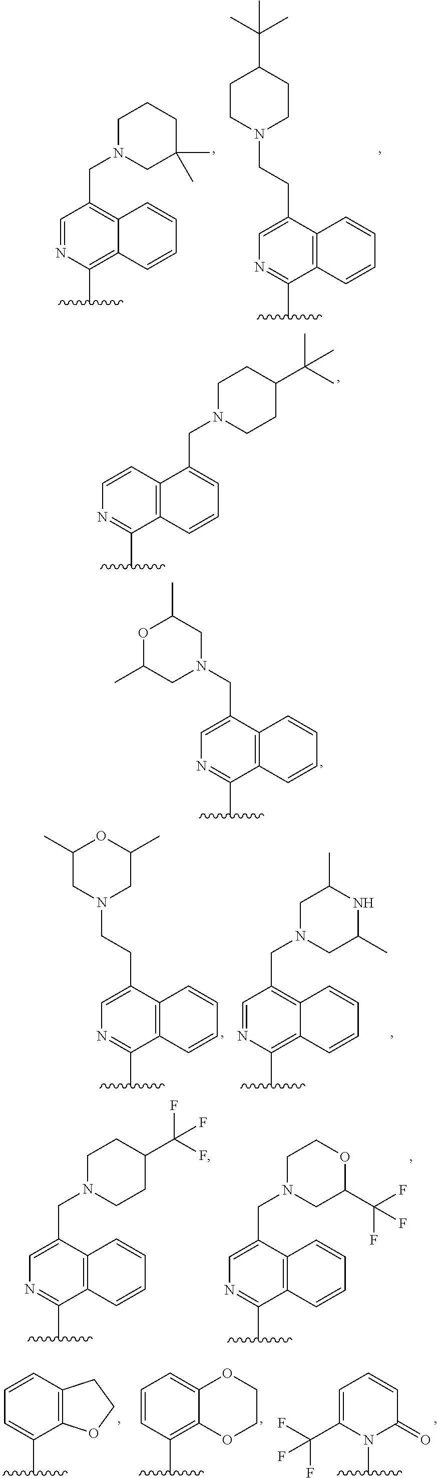

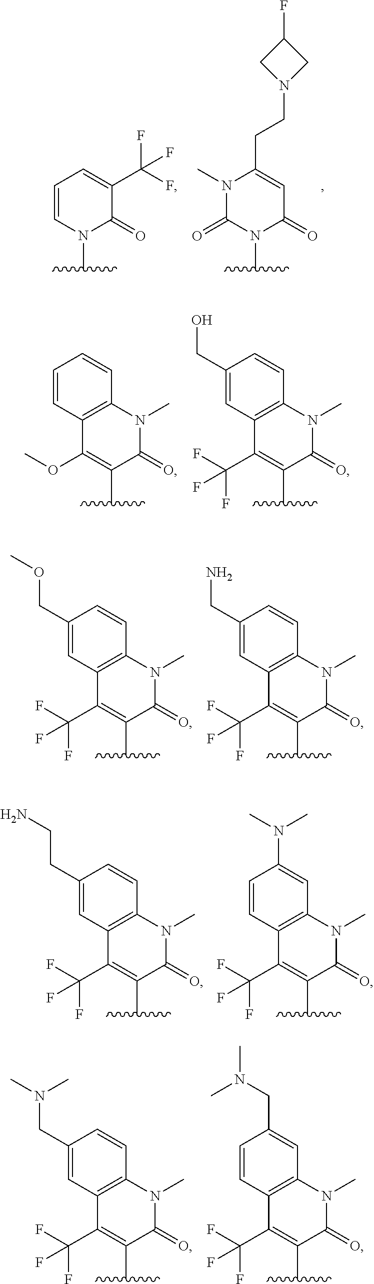

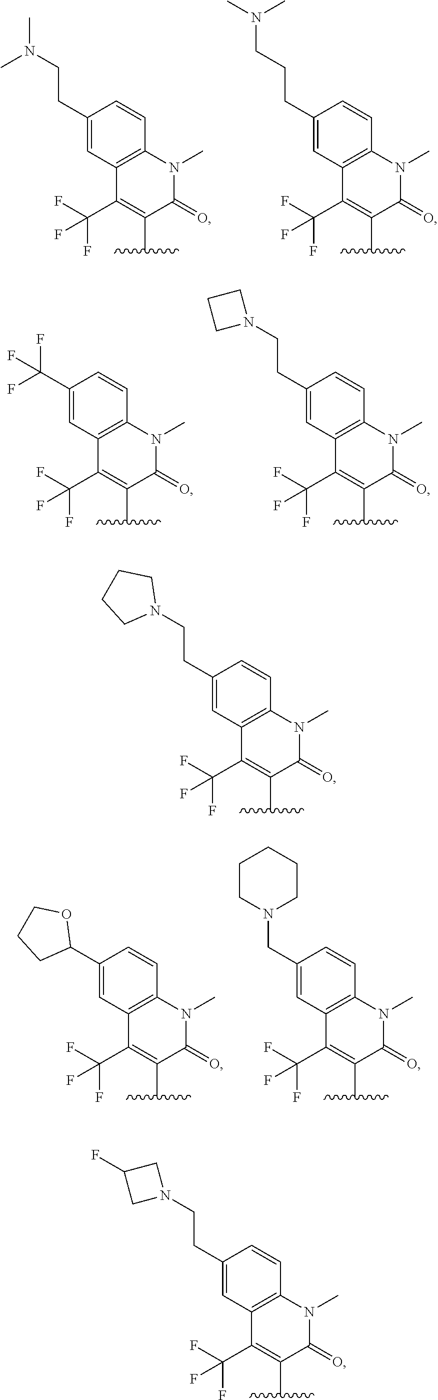

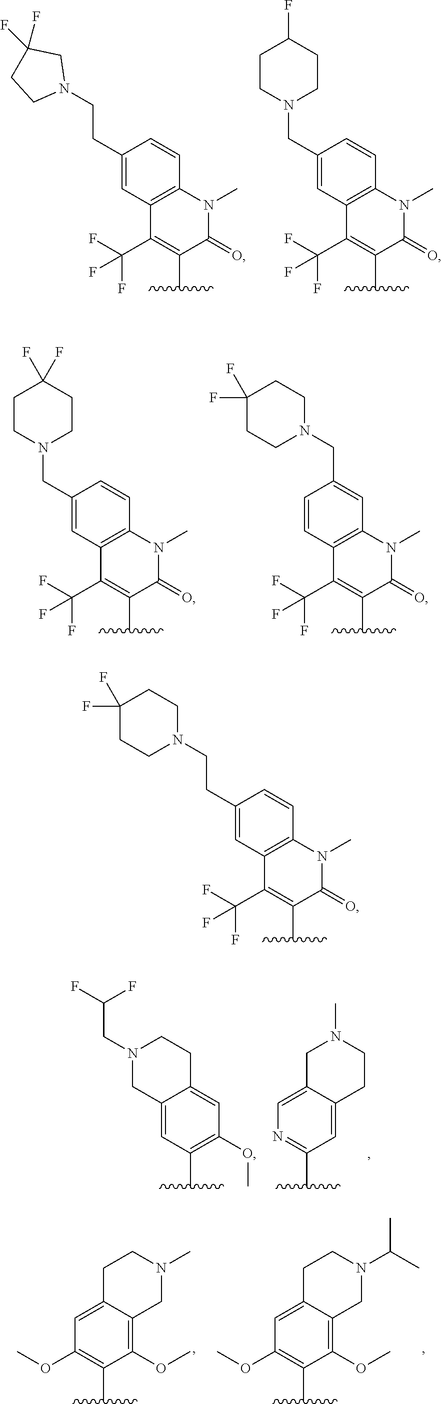

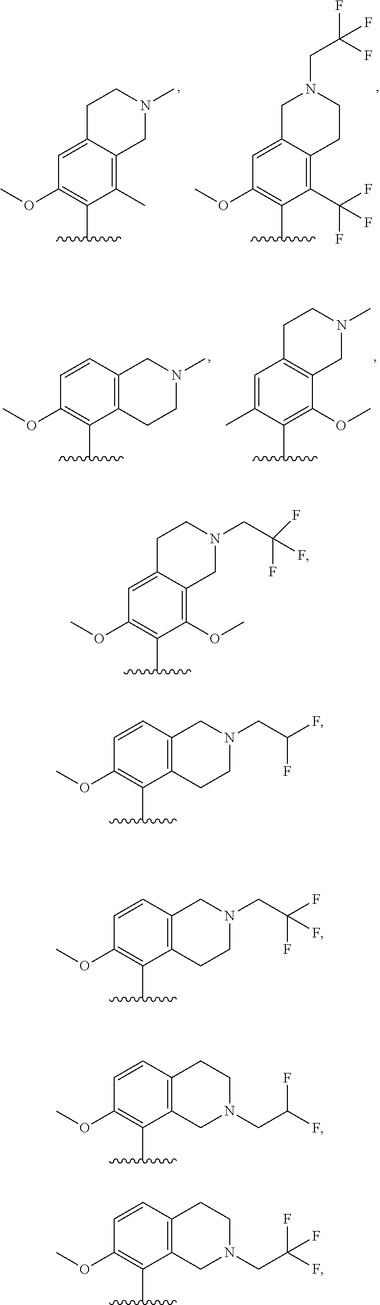

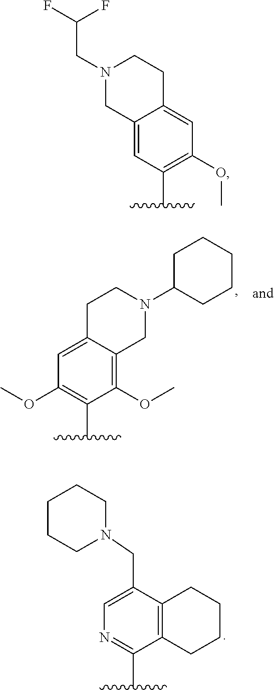

81. The compound or salt of claim 43, wherein the compound or salt of Formula I or Formula (I-a) is selected from a compound or salt of Table 1, Table 2, Table 3, Table 4, Table 14, Table 15, Table 16, Table 17, Table 18, Table 19, Table 20, Table 21, Table 22, Table 23, Table 24, Table 25, Table 26, Table 27, or Table 28.

82. A pharmaceutical composition comprising pharmaceutically acceptable excipient and a compound or salt of claim 1.

83. A method of modulating alpha 4 beta 7 integrin in a subject in need thereof, comprising administering to the subject a compound or salt of claim 81 or a pharmaceutical composition of claim 82.

84. A method of treating an inflammatory disease or condition comprising administering to a subject in need thereof a compound or salt of claim 81 or a pharmaceutical composition of claim 82.

85. The method of claim 84, wherein the inflammatory disease or condition is selected from: inflammatory bowel disease, ulcerative colitis, Crohn's disease, graft-versus-host disease, type 1 diabetes, immune-mediated colitis, checkpoint inhibitor induced colitis, and primary sclerosing cholangitis.
